(19) United States
(12) Patent Application Publication
(10) Pub. No.: US 2022/0098585 A1
(43) Pub. Date: Mar. 31, 2022

(54) OLIGONUCLEOTIDE COMPOSITIONS AND METHODS THEREOF

(71) Applicant: WAVE LIFE SCIENCES LTD., Singapore (SG)

(72) Inventors: Jeffrey Matthew Brown, Medway, MA (US); Shaunna Syu-Mei Berkovitch, Arlington, MA (US); Naoki Iwamoto, Boston, MA (US); Chandra Vargeese, Schwenksville, PA (US); Kidist M. Aklilu, Cambridge, MA (US); Maria David Frank-Kamenetsky, Brookline, MA (US); Duncan Parley Brown, Berkeley, CA (US)

(21) Appl. No.: 17/426,511

(22) PCT Filed: Jan. 30, 2020
(86) PCT No.: PCT/US20/15971

§ 371 Date: Jul. 28, 2021

Related U.S. Application Data
(60) Provisional application No. 62/800,409, filed on Feb. 1, 2019.
(60) Provisional application No. 62/911,335, filed on Oct. 6, 2019.

(51) Int. Cl. C12N 15/113

(52) U.S. Cl. CPC C12N 15/113; C12N 2320/30; C12N 2310/321; C12N 2310/14; C12N 2310/315

(57) ABSTRACT

Among other things, the present disclosure provides oligonucleotides, compositions, and methods for preventing and/or treating various conditions, disorders or diseases. In some embodiments, provided oligonucleotides comprise nucleobase modifications, sugar modifications, internucleotidic linkage modifications and/or patterns thereof, and have improved properties, activities and/or selectivities. In some embodiments, the present disclosure provides oligonucleotides, compositions and methods for HTT-related conditions, disorders or diseases, such as Huntington's disease.

### CROSS-REFERENCE TO RELATED APPLICATIONS

This application claims priority to U.S. Provisional Application No. 62/800,409, filed Feb. 1, 2019, and 62/911,335, filed Oct. 6, 2019, the entirety of each of which is incorporated herein by reference.

### BACKGROUND

Oligonucleotides targeting a particular gene are useful in various applications, e.g., therapeutic, diagnostic, and/or research applications, including but not limited to treatment of various disorders related to the target gene.

### SUMMARY

In some embodiments, the present disclosure provides oligonucleotides and compositions thereof that have significantly improved properties and/or activities. Among other things, the present disclosure provides technologies for designing, manufacturing and utilizing such oligonucleotides and compositions. Particularly, in some embodiments, the present disclosure provides useful patterns of internucleotidic linkages [e.g., types, modifications, and/or configuration (Rp or Sp) of chiral linkage phosphorus, etc.] and/or patterns of sugar modifications (e.g., types, patterns, etc.), which, when combined with one or more other structural elements described herein, e.g., base sequence (or portion thereof), nucleobase modifications (and patterns thereof), internucleotidic linkage modifications (and patterns thereof), additional chemical moieties, etc., can provide oligonucleotides and compositions with high activities and/or desired properties, including but not limited to allele-specific knockdown of mutant allele of a HTT (Huntingtin) gene, wherein the mutant allele is on the same chromosome as (in phase with) an expanded CAG repeat region associated with Huntington's Disease.

In some embodiments, a target HTT nucleic acid is a mutant that comprises both a differentiating position and mutation such as an expanded CAG repeat region (e.g., greater than about 36 CAG), which is associated with Huntington's Disease. In some embodiments, a reference or non-target HTT nucleic acid is wild-type and comprises a different variant of a differentiating position and lacks an expanded CAG repeat region (e.g., the CAG repeat region is less than about 35 CAG and is not associated with Huntington's Disease. In some embodiments, a HTT oligonucleotide (an oligonucleotide that targets a HTT target HTT nucleic acid) is capable of differentiating the target HTT nucleic acid and the reference HTT nucleic acid, and is capable of mediating allele-specific knockdown of the target HTT nucleic acid. In some embodiments, a differentiating position is a single-nucleotide polymorphism (SNP) site, point mutation, etc. In some embodiments, a target HTT nucleic acid sequence and a reference HTT nucleic acid sequence comprise a different base at a SNP site. In some embodiments, a site in a target HTT nucleic acid is fully complementary to a site in an oligonucleotide of the present disclosure while the corresponding site in a reference HTT nucleic acid is not. For example, in some embodiments, a target HTT nucleic acid sequence comprises rs362273 and is A at this SNP position, and its allele comprises expanded CAG repeats (e.g., 36 or more) and it is associated with Huntington's disease; a reference HTT nucleic acid sequence comprises rs362273 and is G at this SNP position, and its allele comprises fewer CAG repeats (e.g., 35 or fewer) and it is less or is not associated with Huntington disease. In some embodiments, sequences of provided oligonucleotides, e.g., GUUGATCTGTAGCAGCAGCT, are complementary to a target HTT nucleic acid sequence at a particular site, e.g., a SNP site (e.g., for GUUGATCTGTAGCAGCAGCT, T is complementary to A at the SNP rs362273 position).

In some embodiments, a HTT oligonucleotide has a base sequence which is not different in a target mutant HTT nucleic acid and a wild-type HTT nucleic acid. In some embodiments, such an oligonucleotide is capable of knocking down the level, expression and/or activity of both a mutant and a wild-type HTT; and the oligonucleotide may be designed as a pan-specific oligonucleotide or non-allele-specific oligonucleotide.

In some embodiments, provided oligonucleotides and compositions are useful for preventing and/or treating various conditions, disorders or diseases, particularly HTT-related conditions, disorders or diseases, including Huntington's Disease. In some embodiments, provided oligonucleotides and compositions selectively reduce levels of HTT transcripts and/or products encoded thereby that are associated with Huntington's Disease. In some embodiments, provided oligonucleotides and compositions selectively reduce levels of HTT transcripts comprising expanded CAG repeats (e.g., 36 or more) and/or products encoded thereby.

Among other things, the present disclosure encompasses the recognition that controlling structural elements of HTT oligonucleotides can have a significant impact on oligonucleotide properties and/or activities, including knockdown (e.g., a decrease in the activity, expression and/or level) of an HTT target gene (or a product thereof). In some embodiments, Huntington's Disease is associated with the presence of a mutant HTT allele which comprises a CAG expansion (e.g., an increase in the length of the region comprising multiple CAG repeats). In some embodiments, knockdown is allele-specific (wherein the mutant allele of HTT is preferentially knocked down relative to the wild-type). In some embodiments, the knockdown is pan-specific (wherein both the mutant and wild-type alleles of HTT are significantly knocked down). In some embodiments, knockdown of an HTT target gene is mediated by RNase H and/or steric hindrance affecting translation. In some embodiments, knockdown of an HTT target gene is mediated by a mechanism involving RNA interference. In some embodiments, controlled structural elements of HTT oligonucleotides include but are not limited to: base sequence, chemical modifications (e.g., modifications of a sugar, base and/or internucleotidic linkage) or patterns thereof, alterations in stereochemistry (e.g., stereochemistry of a backbone chiral internucleotidic linkage) or patterns thereof, structure of a first or second wing or core, and/or conjugation with an additional chemical moiety (e.g., a carbohydrate moiety, a targeting moiety, etc.). Particularly, in some embodiments, the present disclosure demonstrates that control of stereochemistry of backbone chiral centers (stereochemistry of linkage phosphorus), optionally with controlling other aspects of oligonucleotide design and/or incorporation of carbohydrate moieties, can greatly improve properties and/or activities of HTT oligonucleotides.

In some embodiments, the present disclosure pertains to any HTT oligonucleotide which operates through any mechanism, and which comprises any sequence, structure or format (or portion thereof) described herein, wherein the oligonucleotide comprises at least one non-naturally-occurring modification of a base, sugar or internucleotidic linkage.

In some embodiments, the present disclosure provides a oligonucleotide composition comprising a plurality of oligonucleotides, wherein the oligonucleotides comprise at least one chirally controlled internucleotidic linkage [an internucleotidic linkage whose linkage phosphorus is in or is enriched for the Rp or Sp configuration (e.g., 80-100%, 85%-100%, 90%-100%, 95%-100%, or 85%, 90%, 91%, 92%, 93%, 94%, 95%, 96%, 97%, 98%, 99% or more of all oligonucleotides of the same constitution in the composition share the same stereochemistry at the linkage phosphorus) but not a random mixture of the Rp and Sp, such an internucleotidic linkage also a “stereodefined internucleotidic linkage”], e.g., a phosphorothioate linkage whose linkage phosphorus is Rp or Sp. In some embodiments, the number of chirally controlled internucleotidic linkages is 1-100, 1-50, 1-40, 1-35, 1-30, 1-25, 1-20, 5-100, 5-50, 5-40, 5-35, 5-30, 5-25, 5-20, 1, 2, 3, 4, 5, 6, 7, 8, 9, 10, 11, 12, 13, 14, 15, 16, 17, 18, 19, 20, 21, 22, 23, 24, or 25. In some embodiments, at least 1 internucleotidic linkage is chirally controlled internucleotidic linkage and is Sp, and/or at least 1 internucleotidic linkage is chirally controlled internucleotidic linkage and are Rp. In some embodiments, pattern of backbone chiral centers of an oligonucleotide or a portion thereof (e.g., a core) is or comprises Rp(Sp)₂. In some embodiments, pattern of backbone chiral centers of an oligonucleotide or a portion thereof (e.g., a core) is or comprises (Np)t[(Rp)n(Sp)m]y, wherein each of t, n, m, and y is independently as described herein.

In some embodiments, the present disclosure demonstrates that oligonucleotides comprising an Rp chirally controlled internucleotidic linkage at a −1, +1 or +3 position relative to a differentiating position (a position whose base or whose complementary base can differentiate a target mutant HTT nucleic acid and a reference wild-type HTT nucleic acid) can provide high activities and/or selectivities and, in some embodiments, can be particularly useful for reducing levels of disease-associated transcripts and/or products encoded thereby. Unless otherwise specified, for Rp internucleotidic linkage positioning, “−” is counting from the nucleoside at a differentiating position toward the 5′-end of an oligonucleotide with the internucleotidic linkage at the −1 position being the internucleotidic linkage bonded to the 5′-carbon of the nucleoside at the differentiating position, and “+” is counting from the nucleoside at a differentiating position toward the 3′-end of an oligonucleotide with the internucleotidic linkage at the +1 position being the internucleotidic linkage bonded to the 3′-carbon of the nucleoside at the differentiating position. In some embodiments, Rp at −1 position provided increased activity and selectivity. In some embodiments, Rp at +1 position provided increased activity and selectivity. In some embodiments, Rp at +3 position provided increased activity. For example, as shown herein, HTT oligonucleotides WV-12281 (one phosphorothioate in the Rp configuration at position −1 relative to the SNP position), WV-12282 (+1), and WV-12284 (+3) can provide high selectivity when utilized in allele-specific knockdown of the mutant allele.

In some embodiments, the present disclosure pertains to an HTT oligonucleotide composition wherein the HTT oligonucleotides comprise at least one chiral internucleotidic linkage which is not chirally controlled.

In some embodiments, oligonucleotides comprise one or more (e.g., 1, 2, 3, 4, 5, 6, 7, 8, 9, or 10) non-negatively charged internucleotidic linkages. In some embodiments, oligonucleotides comprise one or more neutral internucleotidic linkages. In some embodiments, an HTT oligonucleotide comprises a non-negatively charged or neutral internucleotidic linkage. In some embodiments, the present disclosure provides an oligonucleotide, wherein the base sequence of the oligonucleotide comprises at least 10 contiguous bases of a base sequence that is identical to or complementary to a base sequence of an HTT gene or a transcript thereof, wherein the oligonucleotide comprises at least one non-negatively charged internucleotidic linkage, and wherein the oligonucleotide is capable of decreasing the level, expression and/or activity of an HTT target gene or a gene product thereof.

In some embodiments, the present disclosure encompasses the recognition that various optional additional chemical moieties, such as carbohydrate moieties, targeting moieties, etc., when incorporated into oligonucleotides, can improve one or more properties and/or activities.

In some embodiments, an additional chemical moiety is selected from: GalNAc, glucose, GluNAc (N-acetyl amine glucosamine) and anisamide moieties and derivatives thereof, or any additional chemical moiety described herein and/or known in the art. In some embodiments, an oligonucleotide can comprise two or more additional chemical moieties, wherein the additional chemical moieties are identical or non-identical, or are of the same category (e.g., carbohydrate moiety, sugar moiety, targeting moiety, etc.) or not of the same category. In some embodiments, certain additional chemical moieties facilitate delivery of oligonucleotides to desired cells, tissues and/or organs; and/or facilitate internalization of oligonucleotides; and/or increase oligonucleotide stability.

In some embodiments, the present disclosure provides a chirally controlled oligonucleotide composition comprising a plurality of oligonucleotides which share:

1) a common base sequence;

2) a common pattern of backbone linkages; and

3) a common pattern of backbone chiral centers, which composition is a substantially pure preparation of a single oligonucleotide in that a non-random or controlled level of the oligonucleotides in the composition have the common base sequence, the common pattern of backbone linkages, and the common pattern of backbone chiral centers.

In some embodiments, an oligonucleotide composition is a chirally controlled oligonucleotide composition comprising a plurality of oligonucleotides of a particular oligonucleotide type, which composition is chirally controlled in that it is enriched, relative to a substantially racemic preparation of oligonucleotides having the same base sequence and pattern of chiral internucleotidic linkages, for oligonucleotides of the particular oligonucleotide type.

In some embodiments, the present disclosure provides a chirally controlled oligonucleotide composition comprising a plurality of oligonucleotides capable of directing HTT knockdown, wherein oligonucleotides of the plurality are of a particular oligonucleotide type, which composition is chirally controlled in that it is enriched, relative to a substantially racemic preparation of oligonucleotides having the same base sequence, for oligonucleotides of the particular oligonucleotide type.

In some embodiments, a provided oligonucleotide comprises one or more blocks. In some embodiments, a block comprises one or more consecutive nucleosides, and/or nucleotides, and/or sugars, or bases, and/or internucleotidic linkages which share a common chemistry (e.g., at least one common modification of sugar, base or internucleotidic linkage, or combination or pattern thereof, or pattern of stereochemistry) which is not present in an adjacent block, or vice versa. In some embodiments, an HTT oligonucleotide comprises three or more blocks, wherein the blocks on either end are not identical and the oligonucleotide is thus asymmetric. In some embodiments, a block is a wing or a core. In some embodiments, a core is also referenced to as a gap.

In some embodiments, an oligonucleotide comprises at least one wing and at least one core, wherein a wing differs structurally from a core in that a wing of an oligonucleotide comprises a structure [e.g., stereochemistry, or chemical modification at a sugar, base or internucleotidic linkage (or pattern thereof), etc.] not present in the core, or vice versa. In some embodiments, the structure of an oligonucleotide comprises a wing-core-wing structure. In some embodiments, the structure of an oligonucleotide comprises a wing-core, core-wing, or wing-core-wing structure, wherein one wing differs in structure [e.g., stereochemistry, additional chemical moiety, or chemical modification at a sugar, base or internucleotidic linkage (or pattern thereof)] from the other wing and the core (for example, an asymmetrical oligonucleotide).

In some embodiments, a wing comprises a sugar modification or a pattern thereof that is absent from a core. In some embodiments, a wing comprises a sugar modification that is absent from a core. In some embodiments, one or more (e.g., 1, 2, 3, 4, 5, 6, 7, 8, 9, or 10) sugars of a wing is/are independently modified. In some embodiments, each wing sugar is independently modified. In some embodiments, each sugar in a wing is the same. In some embodiments, at least one sugar in a wing is different from another sugar in the wing. In some embodiments, one or more sugar modifications and/or patterns of sugar modifications in a first wing of an oligonucleotide (e.g., a 5′-wing) is/are different from one or more sugar modifications and/or patterns of sugar modifications in a second wing of the oligonucleotide (e.g., a 3′-wing). In some embodiments, a modification is a 2′-OR modification, wherein R is as described herein. In some embodiments, R is optionally substituted C₁₋₄ alkyl. In some embodiments, a modification is 2′-OMe. In some embodiments, a modification is a 2′-MOE. In some embodiments, a modified sugar is a high-affinity sugar, e.g., a bicyclic sugar (e.g., a LNA sugar), 2′-MOE, etc. In some embodiments, a sugar of a 3′-wing is a high-affinity sugar. In some embodiments, a 3′-wing comprises one or more high-affinity sugars. In some embodiments, each sugar of a 3′-wing is independently a high-affinity sugar. In some embodiments, a high-affinity sugar is a 2′-MOE sugar. In some embodiments, a high-affinity sugar is bonded to a non-negatively charged internucleotidic linkage.

In some embodiments, a wing comprises one or more non-negatively charged internucleotidic linkages. In some embodiments, a non-negatively charged internucleotidic linkage is a neutral internucleotidic linkage. In some embodiments, each non-negatively charged internucleotidic linkage is independently a neutral internucleotidic linkage. In some embodiments, as demonstrated herein, oligonucleotides that comprise wings comprising one or more non-negatively charged internucleotidic linkages can deliver high activities and/or selectivities. In some embodiments, for description of internucleotidic linkages and patterns thereof (including stereochemical patterns), internucleotidic linkages linking a wing nucleoside and a core nucleoside is considered part of the core. In some embodiments, a non-negatively charged internucleotidic linkage is chirally controlled and is Rp or Sp.

In some embodiments, a core sugar is a natural DNA sugar which comprises no substitution at the 2′ position (two —H at 2′-carbon). In some embodiments, each core sugar is a natural DNA sugar which comprises no substitution at the 2′ position (two —H at 2′-carbon).

In some embodiments, a differentiating position (e.g., a SNP location or other mutation which differentiates a wild-type target sequence from a disease-associated or mutant sequence) is position 4, 5 or 6 from the 5′-end of a core region. In some embodiments, the 4^{th}, 5^{th} or 6^{th} nucleobase of a core region (from the 5′ end of a core) is characteristic of a sequence and differentiates a sequence from another sequence (e.g., a SNP). In some embodiments, a differentiating position is position 4 from the 5′-end of a core region. In some embodiments, a differentiating position is position 5 from the 5′-end of a core region. In some embodiments, a differentiating position is position 6 from the 5′-end of a core region. In some embodiments, a differentiating position is position 9, 10 or 11 from the 5′-end of an oligonucleotide. In some embodiments, a differentiating position is position 9 from the 5′-end of an oligonucleotide. In some embodiments, a differentiating position is position 10 from the 5′-end of an oligonucleotide. In some embodiments, a differentiating position is position 11 from the 5′-end of an oligonucleotide.

In some embodiments, an oligonucleotide or oligonucleotide composition is useful for preventing or treating a condition, disorder or disease. In some embodiments, an HTT oligonucleotide or HTT oligonucleotide composition is useful for a method of treatment of an HTT-related condition, disorder or disease, such as Huntington's Disease, in a subject in need thereof.

In some embodiments, an oligonucleotide or oligonucleotide composition is useful for the manufacture of a medicament for treatment of a condition, disorder or disease, such as Huntington's Disease, in a subject in need thereof. In some embodiments, an HTT oligonucleotide or HTT oligonucleotide composition is useful for the manufacture of a medicament for treatment of an HTT-related condition, disorder or disease, such as Huntington's Disease, in a subject in need thereof.

### BRIEF DESCRIPTION OF THE DRAWINGS

FIGS. 1A-1D. FIGS. 1A-1D shows various formats which can be used, in whole or in part, for oligonucleotides, e.g., HTT oligonucleotides.

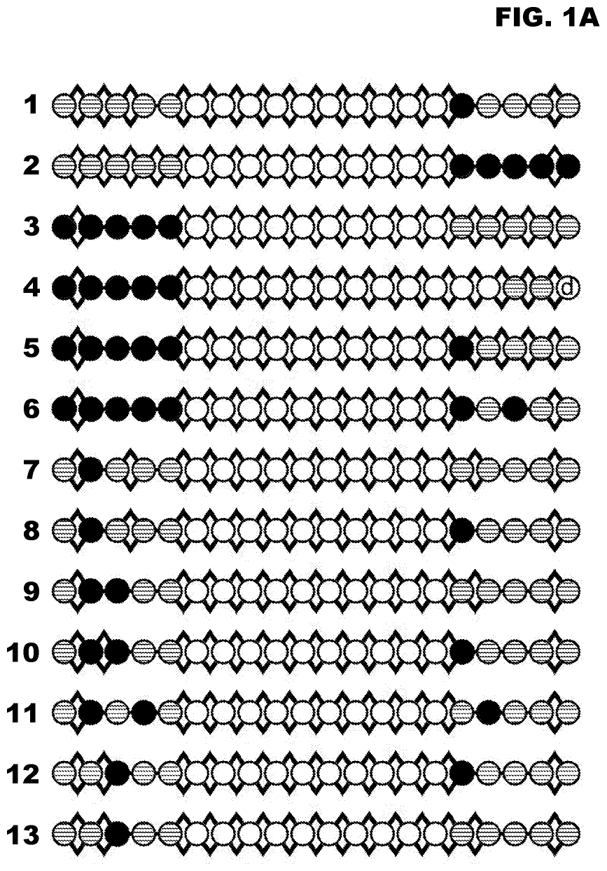

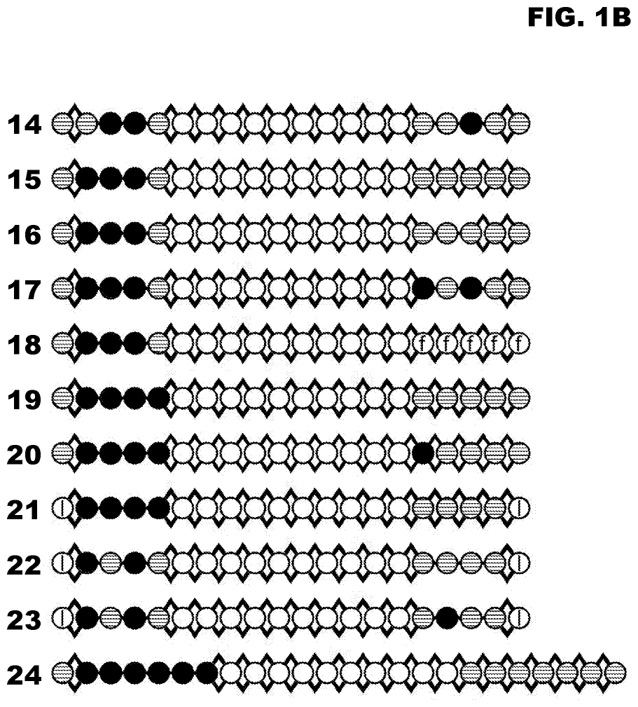

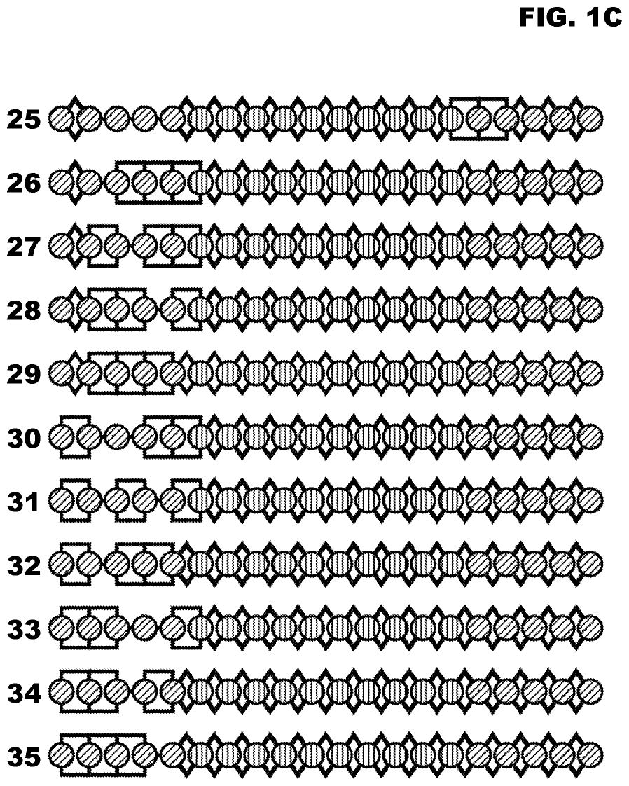

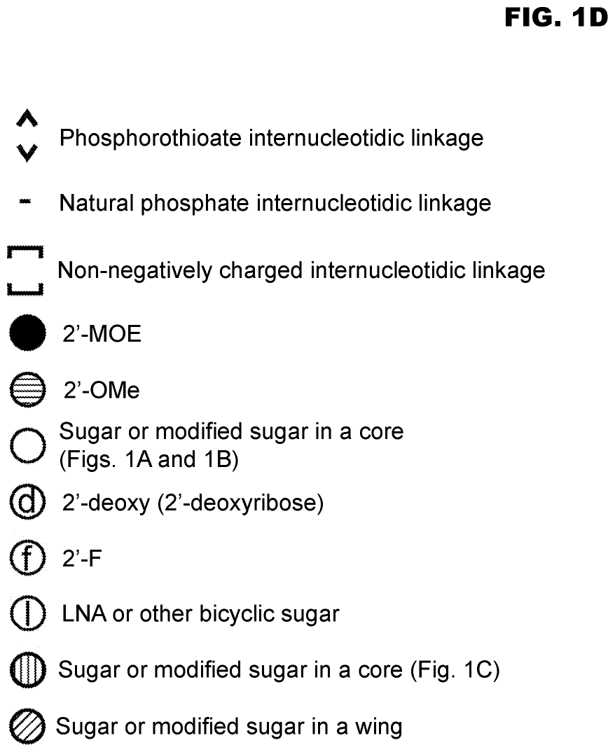

### DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

Unless otherwise specified, description of oligonucleotides and elements thereof (e.g., base sequence, sugar modifications, internucleotidic linkages, linkage phosphorus stereochemistry, etc.) is from 5′ to 3′. Unless otherwise specified, oligonucleotides described herein may be provided and/or utilized in a salt form, particularly a pharmaceutically acceptable salt form. As those skilled in the art will appreciate after reading the present disclosure, in some embodiments, oligonucleotides may be provided as salts, e.g., sodium salts. As those skilled in the art will appreciate, in some embodiments, individual oligonucleotides within a composition may be considered to be of the same constitution and/or structure even though, within such composition (e.g., a liquid composition), particular such oligonucleotides might be in different salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition)), and will understand that, so long as their acid forms (e.g., replacing all cations, if any, with H) are of the same constitution and/or structure, such individual oligonucleotides may properly be considered to be of the same constitution and/or structure.

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, an alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an aliphatic group, as defined herein, having one or more triple bonds.

Analog: The term “analog” includes any chemical moiety which differs structurally from a reference chemical moiety or class of moieties, but which is capable of performing at least one function of such a reference chemical moiety or class of moieties. As non-limiting examples, a nucleotide analog differs structurally from a nucleotide but performs at least one function of a nucleotide; a nucleobase analog differs structurally from a nucleobase but performs at least one function of a nucleobase; etc.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In some embodiments, animals include, but are not limited to, mammals, birds, reptiles, amphibians, fish and/or worms. In some embodiments, an animal may be a transgenic animal, a genetically-engineered animal and/or a clone.

Antisense: The term “antisense”, as used herein, refers to a characteristic of an oligonucleotide or other nucleic acid having a base sequence complementary or substantially complementary to a target HTT nucleic acid to which it is capable of hybridizing. In some embodiments, a target HTT nucleic acid is a target gene mRNA. In some embodiments, hybridization is required for or results in at one activity, e.g., a decrease in the level, expression or activity of the target HTT nucleic acid or a gene product thereof. The term “antisense oligonucleotide”, as used herein, refers to an oligonucleotide complementary to a target HTT nucleic acid. In some embodiments, an antisense oligonucleotide is capable of directing a decrease in the level, expression or activity of a target HTT nucleic acid or a product thereof. In some embodiments, an antisense oligonucleotide is capable of directing a decrease in the level, expression or activity of the target HTT nucleic acid or a product thereof, via a mechanism that involves RNaseH, steric hindrance and/or RNA interference.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Chiral control: As used herein, “chiral control” refers to control of the stereochemical designation of the chiral linkage phosphorus in a chiral internucleotidic linkage within an oligonucleotide. As used herein, a chiral internucleotidic linkage is an internucleotidic linkage whose linkage phosphorus is chiral. In some embodiments, a control is achieved through a chiral element that is absent from the sugar and base moieties of an oligonucleotide, for example, in some embodiments, a control is achieved through use of one or more chiral auxiliaries during oligonucleotide preparation as described in the present disclosure, which chiral auxiliaries often are part of chiral phosphoramidites used during oligonucleotide preparation. In contrast to chiral control, a person having ordinary skill in the art appreciates that conventional oligonucleotide synthesis which does not use chiral auxiliaries cannot control stereochemistry at a chiral internucleotidic linkage if such conventional oligonucleotide synthesis is used to form the chiral internucleotidic linkage. In some embodiments, the stereochemical designation of each chiral linkage phosphorus in each chiral internucleotidic linkage within an oligonucleotide is controlled.

Chirally controlled oligonucleotide composition: The terms “chirally controlled oligonucleotide composition”, “chirally controlled nucleic acid composition”, and the like, as used herein, refers to a composition that comprises a plurality of oligonucleotides (or nucleic acids) which share 1) a common base sequence, 2) a common pattern of backbone linkages, and 3) a common pattern of backbone phosphorus modifications, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). Level of the plurality of oligonucleotides (or nucleic acids) in a chirally controlled oligonucleotide composition is pre-determined/controlled (e.g., through chirally controlled oligonucleotide preparation to stereoselectively form one or more chiral internucleotidic linkages). In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition are oligonucleotides of the plurality. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence, the common pattern of backbone linkages, and the common pattern of backbone phosphorus modifications are oligonucleotides of the plurality. In some embodiments, a level is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a composition, or of all oligonucleotides in a composition that share a common base sequence (e.g., of a plurality of oligonucleotide or an oligonucleotide type), or of all oligonucleotides in a composition that share a common base sequence, a common pattern of backbone linkages, and a common pattern of backbone phosphorus modifications, or of all oligonucleotides in a composition that share a common base sequence, a common patter of base modifications, a common pattern of sugar modifications, a common pattern of internucleotidic linkage types, and/or a common pattern of internucleotidic linkage modifications. In some embodiments, the plurality of oligonucleotides share the same stereochemistry at about 1-50 (e.g., about 1-10, 1-20, 5-10, 5-20, 10-15, 10-20, 10-25, 10-30, or about 1, 2, 3, 4, 5, 6, 7, 8, 9, 10, 11, 12, 13, 14, 15, 16, 17, 18, 19, or 20, or at least 1, 2, 3, 4, 5, 6, 7, 8, 9, 10, 11, 12, 13, 14, 15, 16, 17, 18, 19, or 20) chiral internucleotidic linkages. In some embodiments, the plurality of oligonucleotides share the same stereochemistry at about 1%-100% (e.g., about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%) of chiral internucleotidic linkages. In some embodiments, oligonucleotides (or nucleic acids) of a plurality are of the same constitution. In some embodiments, level of the oligonucleotides (or nucleic acids) of the plurality is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides (or nucleic acids) in a composition that share the same constitution as the oligonucleotides (or nucleic acids) of the plurality. In some embodiments, each chiral internucleotidic linkage is a chiral controlled internucleotidic linkage, and the composition is a completely chirally controlled oligonucleotide composition. In some embodiments, oligonucleotides (or nucleic acids) of a plurality are structurally identical. In some embodiments, a chirally controlled internucleotidic linkage has a diastereopurity of at least 80%, 85%, 90%, 91%, 92%, 93%, 94%, 95%, 96%, 97%, 98%, 99% or 99.5%, typically at least 90%, 91%, 92%, 93%, 94%, 95%, 96%, 97%, 98%, 99% or 99.5%. In some embodiments, a chirally controlled internucleotidic linkage has a diastereopurity of at least 95%. In some embodiments, a chirally controlled internucleotidic linkage has a diastereopurity of at least 96%. In some embodiments, a chirally controlled internucleotidic linkage has a diastereopurity of at least 97%. In some embodiments, a chirally controlled internucleotidic linkage has a diastereopurity of at least 98%. In some embodiments, a chirally controlled internucleotidic linkage has a diastereopurity of at least 99%. In some embodiments, a percentage of a level is or is at least (DS)^{nc}, wherein DS is a diastereopurity as described in the present disclosure (e.g., 90%, 91%, 92%, 93%, 94%, 95%, 96%, 97%, 98%, 99% or 99.5% or more) and nc is the number of chirally controlled internucleotidic linkages as described in the present disclosure (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In some embodiments, a percentage of a level is or is at least (DS)^{nc}, wherein DS is 95%-100%. For example, when DS is 99% and nc is 10, the percentage is or is at least 90% ((99%)¹⁰≈0.90=90%). In some embodiments, level of a plurality of oligonucleotides in a composition is represented as the product of the diastereopurity of each chirally controlled internucleotidic linkage in the oligonucleotides. In some embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide . . . NxNy . . . , the dimer is NxNy). In some embodiments, not all chiral internucleotidic linkages are chiral controlled internucleotidic linkages, and the composition is a partially chirally controlled oligonucleotide composition. In some embodiments, a non-chirally controlled internucleotidic linkage has a diastereopurity of less than about 80%, 75%, 70%, 65%, 60%, 55%, or of about 50%, as typically observed in stereorandom oligonucleotide compositions (e.g., as appreciated by those skilled in the art, from traditional oligonucleotide synthesis, e.g., the phosphoramidite method). In some embodiments, oligonucleotides (or nucleic acids) of a plurality are of the same type. In some embodiments, a chirally controlled oligonucleotide composition comprises non-random or controlled levels of individual oligonucleotide or nucleic acids types. For instance, in some embodiments a chirally controlled oligonucleotide composition comprises one and no more than one oligonucleotide type. In some embodiments, a chirally controlled oligonucleotide composition comprises more than one oligonucleotide type. In some embodiments, a chirally controlled oligonucleotide composition comprises multiple oligonucleotide types. In some embodiments, a chirally controlled oligonucleotide composition is a composition of oligonucleotides of an oligonucleotide type, which composition comprises a non-random or controlled level of a plurality of oligonucleotides of the oligonucleotide type.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Gapmer: as used herein, the term “gapmer” refers to an oligonucleotide characterized in that it comprises a core flanked by a 5′ and a 3′ wing. In some embodiments, in a gapmer, at least one internucleotidic phosphorus linkage of the oligonucleotide is a natural phosphate linkage. In some embodiments, more than one internucleotidic phosphorus linkage of the oligonucleotide strand is a natural phosphate linkage. In some embodiments, a gapmer is a sugar modification gapmer, wherein each wing sugar independently comprises a sugar modification, and no core sugar comprises a sugar modification found in a wing sugar. In some embodiments, each core sugar comprises no modification and are 2′-unsubstituted (as in natural DNA). In some embodiments, each wing sugar is independently a 2′-modified sugar. In some embodiments, at least one wing sugar is a bicyclic sugar. In some embodiments, sugar units in each wing have the same sugar modification (e.g., 2′-OMe (a 2′-OMe wing), 2′-MOE (a 2′-MOE wing), etc.). In some embodiments, each wing sugar has the same modification. Core and wing can have various lengths. In some embodiments, a wing is 2, 3, 4, 5, 6, 7, 8, 9, 10 or more nucleosides (in many embodiments, 3, 4, 5, or 6 or more) in length, and a core is 2, 3, 4, 5, 6, 7, 8, 9, 10, 11, 12, 13, 14, 15, 16, 17, 18, 19, 20 or more nucleosides (in many embodiments, 8, 9, 10, 11, 12, or more) in length. In some embodiments, an oligonucleotide comprises or consists of a wing-core-wing structure of 2-9-6, 3-9-3, 3-9-4, 3-9-5, 4-7-4, 4-9-4, 4-9-5, 4-10-5, 4-11-4, 4-11-5, 5-7-5, 5-8-6, 5-9-3, 5-9-5, 5-10-4, 5-10-5, 6-7-6, 6-8-5, or 6-9-2. In some embodiments, an oligonucleotide is a gapmer.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including any oxidized form of nitrogen, sulfur, phosphorus, or silicon; the quaternized form of any basic nitrogen or a substitutable nitrogen of a heterocyclic ring (for example, N as in 3,4-dihydro-2H-pyrrolyl), NH (as in pyrrolidinyl) or NR⁺ (as in N-substituted pyrrolidinyl); etc.); in some embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Homology: “Homology” or “identity” or “similarity” refers to sequence similarity between two nucleic acid molecules. Homology and identity can each be determined by comparing a position in each sequence which can be aligned for purposes of comparison. When an equivalent position in the compared sequences is occupied by the same base, then the molecules are identical at that position; when the equivalent site occupied by the same or a similar nucleic acid residue (e.g., similar in steric and/or electronic nature), then the molecules can be referred to as homologous (similar) at that position. Expression as a percentage of homology/similarity or identity refers to a function of the number of identical or similar nucleic acids at positions shared by the compared sequences. In some embodiments, a sequence which is “unrelated” or “non-homologous” shares less than 40% identity, less than 35% identity, less than 30% identity, or less than 25% identity with a sequence described herein. In comparing two sequences, the absence of residues (amino acids or nucleic acids) or presence of extra residues also decreases the identity and homology/similarity. In some embodiments, polymeric molecules (e.g., oligonucleotides, nucleic acids, proteins, etc.) are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

In some embodiments, the term “homology” describes a mathematically based comparison of sequence similarities which is used to identify genes with similar functions or motifs. The nucleic acid sequences described herein can be used as a “query sequence” to perform a search against public databases, for example, to identify other family members, related sequences or homologs. In some embodiments, such searches can be performed using the NBLAST and XBLAST programs (version 2.0) of Altschul, et al. (1990) J. Mol. Biol. 215:403-10. In some embodiments, BLAST nucleotide searches can be performed with the NBLAST program, score=100, wordlength=12 to obtain nucleotide sequences homologous to nucleic acid molecules of the disclosure. In some embodiments, to obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and BLAST) can be used (See www.ncbi.nlm.nih.gov).

Identity: As used herein, the term “identity” refers to the overall relatedness between polymeric molecules, e.g., between nucleic acid molecules (e.g., oligonucleotides, DNA, RNA, etc.) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Internucleotidic linkage: As used herein, the phrase “internucleotidic linkage” refers generally to a linkage linking nucleoside units of an oligonucleotide or a nucleic acid. In some embodiments, an internucleotidic linkage is a phosphodiester linkage, as extensively found in naturally occurring DNA and RNA molecules (natural phosphate linkage (—OP(═O)(OH)O—), which as appreciated by those skilled in the art may exist as a salt form). In some embodiments, an internucleotidic linkage is a modified internucleotidic linkage (not a natural phosphate linkage). In some embodiments, an internucleotidic linkage is a “modified internucleotidic linkage” wherein at least one oxygen atom or —OH of a phosphodiester linkage is replaced by a different organic or inorganic moiety. In some embodiments, such an organic or inorganic moiety is selected from ═S, ═Se, ═NR′, —SR′, —SeR′, —N(R′)₂, B(R′)₃, —S—, —Se—, and —N(R′)—, wherein each R′ is independently as defined and described in the present disclosure. In some embodiments, an internucleotidic linkage is a phosphotriester linkage, phosphorothioate linkage (or phosphorothioate diester linkage, —OP(═O)(SH)O—, which as appreciated by those skilled in the art may exist as a salt form), or phosphorothioate triester linkage. In some embodiments, a modified internucleotidic linkage is a phosphorothioate linkage. In some embodiments, an internucleotidic linkage is one of, e.g., PNA (peptide nucleic acid) or PMO (phosphorodiamidate Morpholino oligomer) linkage. In some embodiments, a modified internucleotidic linkage is a non-negatively charged internucleotidic linkage. In some embodiments, a modified internucleotidic linkage is a neutral internucleotidic linkage (e.g., n001 in certain provided oligonucleotides). It is understood by a person of ordinary skill in the art that an internucleotidic linkage may exist as an anion or cation at a given pH due to the existence of acid or base moieties in the linkage. In some embodiments, a modified internucleotidic linkages is a modified internucleotidic linkages designated as s, s1, s2, s3, s4, s5, s6, s7, s8, s9, s10, s11, s12, s13, s14, s15, s16, s17 and s18 as described in WO 2017/210647.

In vitro: As used herein, the term “in vitro” refers to events that occur in an artificial environment, e.g., in a test tube or reaction vessel, in cell culture, etc., rather than within an organism (e.g., animal, plant and/or microbe).

In vivo: As used herein, the term “in vivo” refers to events that occur within an organism (e.g., animal, plant and/or microbe).

Linkage phosphorus: as defined herein, the phrase “linkage phosphorus” is used to indicate that the particular phosphorus atom being referred to is the phosphorus atom present in the internucleotidic linkage, which phosphorus atom corresponds to the phosphorus atom of a phosphodiester internucleotidic linkage as occurs in naturally occurring DNA and RNA. In some embodiments, a linkage phosphorus atom is in a modified internucleotidic linkage, wherein each oxygen atom of a phosphodiester linkage is optionally and independently replaced by an organic or inorganic moiety. In some embodiments, a linkage phosphorus atom is the P of Formula I as defined herein. In some embodiments, a linkage phosphorus atom is chiral. In some embodiments, a linkage phosphorus atom is achiral (e.g., as in natural phosphate linkages).

Linker: The terms “linker”, “linking moiety” and the like refer to any chemical moiety which connects one chemical moiety to another. As appreciated by those skilled in the art, a linker can be bivalent or trivalent or more, depending on the number of chemical moieties the linker connects. In some embodiments, a linker is a moiety which connects one oligonucleotide to another oligonucleotide in a multimer. In some embodiments, a linker is a moiety optionally positioned between the terminal nucleoside and the solid support or between the terminal nucleoside and another nucleoside, nucleotide, or nucleic acid. In some embodiments, in an oligonucleotide a linker connects a chemical moiety (e.g., a targeting moiety, a lipid moiety, a carbohydrate moiety, etc.) with an oligonucleotide chain (e.g., through its 5′-end, 3′-end, nucleobase, sugar, internucleotidic linkage, etc.)

Lower alkyl: The term “lower alkyl” refers to a C₁₋₄ straight or branched alkyl group. Example lower alkyl groups are methyl, ethyl, propyl, isopropyl, butyl, isobutyl, and tert-butyl.

Lower haloalkyl: The term “lower haloalkyl” refers to a C₁₋₄ straight or branched alkyl group that is substituted with one or more halogen atoms.

Modified nucleobase: The terms “modified nucleobase”, “modified base” and the like refer to a chemical moiety which is chemically distinct from a nucleobase, but which is capable of performing at least one function of a nucleobase. In some embodiments, a modified nucleobase is a nucleobase which comprises a modification. In some embodiments, a modified nucleobase is capable of at least one function of a nucleobase, e.g., forming a moiety in a polymer capable of base-pairing to a nucleic acid comprising an at least complementary sequence of bases. In some embodiments, a modified nucleobase is substituted A, T, C, G, or U, or a substituted tautomer of A, T, C, G, or U. In some embodiments, a modified nucleobase in the context of oligonucleotides refer to a nucleobase that is not A, T, C, G or U.

Modified nucleoside: The term “modified nucleoside” refers to a moiety derived from or chemically similar to a natural nucleoside, but which comprises a chemical modification which differentiates it from a natural nucleoside. Non-limiting examples of modified nucleosides include those which comprise a modification at the base and/or the sugar. Non-limiting examples of modified nucleosides include those with a 2′ modification at a sugar. Non-limiting examples of modified nucleosides also include abasic nucleosides (which lack a nucleobase). In some embodiments, a modified nucleoside is capable of at least one function of a nucleoside, e.g., forming a moiety in a polymer capable of base-pairing to a nucleic acid comprising an at least complementary sequence of bases.

Modified nucleotide: The term “modified nucleotide” includes any chemical moiety which differs structurally from a natural nucleotide but is capable of performing at least one function of a natural nucleotide. In some embodiments, a modified nucleotide comprises a modification at a sugar, base and/or internucleotidic linkage. In some embodiments, a modified nucleotide comprises a modified sugar, modified nucleobase and/or modified internucleotidic linkage. In some embodiments, a modified nucleotide is capable of at least one function of a nucleotide, e.g., forming a subunit in a polymer capable of base-pairing to a nucleic acid comprising an at least complementary sequence of bases.

Modified sugar: The term “modified sugar” refers to a moiety that can replace a sugar. A modified sugar mimics the spatial arrangement, electronic properties, or some other physicochemical property of a sugar. In some embodiments, as described in the present disclosure, a modified sugar is substituted ribose or deoxyribose. In some embodiments, a modified sugar comprises a 2′-modification. Examples of useful 2′-modification are widely utilized in the art and described herein. In some embodiments, a 2′-modification is 2′-OR, wherein R is optionally substituted C₁₋₁₀ aliphatic. In some embodiments, a 2′-modification is 2′-OMe. In some embodiments, a 2′-modification is 2′-MOE. In some embodiments, a modified sugar is a bicyclic sugar (e.g., a sugar used in LNA, BNA, etc.). In some embodiments, in the context of oligonucleotides, a modified sugar is a sugar that is not ribose or deoxyribose as typically found in natural RNA or DNA.

Nucleic acid: The term “nucleic acid”, as used herein, includes any nucleotides and polymers thereof. The term “polynucleotide”, as used herein, refers to a polymeric form of nucleotides of any length, either ribonucleotides (RNA) or deoxyribonucleotides (DNA) or a combination thereof. These terms refer to the primary structure of the molecules and, thus, include double- and single-stranded DNA, and double- and single-stranded RNA. These terms include, as equivalents, analogs of either RNA or DNA comprising modified nucleotides and/or modified polynucleotides, such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified internucleotidic linkages. The term encompasses nucleic acids containing any combinations of nucleobases, modified nucleobases, sugars, modified sugars, phosphate bridges or modified internucleotidic linkages. Examples include, and are not limited to, nucleic acids containing ribose moieties, nucleic acids containing deoxyribose moieties, nucleic acids containing both ribose and deoxyribose moieties, nucleic acids containing ribose and modified ribose moieties. Unless otherwise specified, the prefix poly- refers to a nucleic acid containing 2 to about 10,000 nucleotide monomer units and wherein the prefix oligo- refers to a nucleic acid containing 2 to about 200 nucleotide monomer units.

Nucleobase: The term “nucleobase” refers to the parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In some embodiments, a naturally-occurring nucleobases are modified adenine, guanine, uracil, cytosine, or thymine. In some embodiments, a naturally-occurring nucleobases are methylated adenine, guanine, uracil, cytosine, or thymine. In some embodiments, a nucleobase comprises a heteroaryl ring wherein a ring atom is nitrogen, and when in a nucleoside, the nitrogen is bonded to a sugar moiety. In some embodiments, a nucleobase comprises a heterocyclic ring wherein a ring atom is nitrogen, and when in a nucleoside, the nitrogen is bonded to a sugar moiety. In some embodiments, a nucleobase is a “modified nucleobase,” a nucleobase other than adenine (A), guanine (G), uracil (U), cytosine (C), and thymine (T). In some embodiments, a modified nucleobase is substituted A, T, C, G or U. In some embodiments, a modified nucleobase is a substituted tautomer of A, T, C, G, or U. In some embodiments, a modified nucleobases is methylated adenine, guanine, uracil, cytosine, or thymine. In some embodiments, a modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. As used herein, the term “nucleobase” also encompasses structural analogs used in lieu of natural or naturally-occurring nucleotides, such as modified nucleobases and nucleobase analogs. In some embodiments, a nucleobase is optionally substituted A, T, C, G, or U, or an optionally substituted tautomer of A, T, C, G, or U. In some embodiments, a “nucleobase” refers to a nucleobase unit in an oligonucleotide or a nucleic acid (e.g., A, T, C, G or U as in an oligonucleotide or a nucleic acid).

Nucleoside: The term “nucleoside” refers to a moiety wherein a nucleobase or a modified nucleobase is covalently bound to a sugar or a modified sugar. In some embodiments, a nucleoside is a natural nucleoside, e.g., adenosine, deoxyadenosine, guanosine, deoxyguanosine, thymidine, uridine, cytidine, or deoxycytidine. In some embodiments, a nucleoside is a modified nucleoside, e.g., a substituted natural nucleoside selected from adenosine, deoxyadenosine, guanosine, deoxyguanosine, thymidine, uridine, cytidine, and deoxycytidine. In some embodiments, a nucleoside is a modified nucleoside, e.g., a substituted tautomer of a natural nucleoside selected from adenosine, deoxyadenosine, guanosine, deoxyguanosine, thymidine, uridine, cytidine, and deoxycytidine. In some embodiments, a “nucleoside” refers to a nucleoside unit in an oligonucleotide or a nucleic acid.

Nucleoside analog: The term “nucleoside analog” refers to a chemical moiety which is chemically distinct from a natural nucleoside, but which is capable of performing at least one function of a nucleoside. In some embodiments, a nucleoside analog comprises an analog of a sugar and/or an analog of a nucleobase. In some embodiments, a modified nucleoside is capable of at least one function of a nucleoside, e.g., forming a moiety in a polymer capable of base-pairing to a nucleic acid comprising a complementary sequence of bases.

Nucleotide: The term “nucleotide” as used herein refers to a monomeric unit of a polynucleotide that consists of a nucleobase, a sugar, and one or more internucleotidic linkages (e.g., phosphate linkages in natural DNA and RNA). The naturally occurring bases [guanine, (G), adenine, (A), cytosine, (C), thymine, (T), and uracil (U)] are derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleotides are linked via internucleotidic linkages to form nucleic acids, or polynucleotides. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. In some embodiments, a natural nucleotide comprises a naturally occurring base, sugar and internucleotidic linkage. As used herein, the term “nucleotide” also encompasses structural analogs used in lieu of natural or naturally-occurring nucleotides, such as modified nucleotides and nucleotide analogs. In some embodiments, a “nucleotide” refers to a nucleotide unit in an oligonucleotide or a nucleic acid.

Oligonucleotide: The term “oligonucleotide” refers to a polymer or oligomer of nucleotides, and may contain any combination of natural and non-natural nucleobases, sugars, and internucleotidic linkages.

Oligonucleotides can be single-stranded or double-stranded. A single-stranded oligonucleotide can have double-stranded regions (formed by two portions of the single-stranded oligonucleotide) and a double-stranded oligonucleotide, which comprises two oligonucleotide chains, can have single-stranded regions for example, at regions where the two oligonucleotide chains are not complementary to each other. Example oligonucleotides include, but are not limited to structural genes, genes including control and termination regions, self-replicating systems such as viral or plasmid DNA, single-stranded and double-stranded RNAi agents and other RNA interference reagents (RNAi agents or iRNA agents), shRNA, antisense oligonucleotides, ribozymes, microRNAs, microRNA mimics, supermirs, aptamers, antimirs, antagomirs, U1 adaptors, triplex-forming oligonucleotides, G-quadruplex oligonucleotides, RNA activators, immuno-stimulatory oligonucleotides, and decoy oligonucleotides.

Oligonucleotides of the present disclosure can be of various lengths. In particular embodiments, oligonucleotides can range from about 2 to about 200 nucleosides in length. In various related embodiments, oligonucleotides, single-stranded, double-stranded, or triple-stranded, can range in length from about 4 to about 10 nucleosides, from about 10 to about 50 nucleosides, from about 20 to about 50 nucleosides, from about 15 to about 30 nucleosides, from about 20 to about 30 nucleosides in length. In some embodiments, the oligonucleotide is from about 9 to about 39 nucleosides in length. In some embodiments, the oligonucleotide is at least 4, 5, 6, 7, 8, 9, 10, 11, 12, 13, 14, 15, 16, 17, 18, 19, 20, 21, 22, 23, 24, or 25 nucleosides in length. In some embodiments, the oligonucleotide is at least 4 nucleosides in length. In some embodiments, the oligonucleotide is at least 5 nucleosides in length. In some embodiments, the oligonucleotide is at least 6 nucleosides in length. In some embodiments, the oligonucleotide is at least 7 nucleosides in length. In some embodiments, the oligonucleotide is at least 8 nucleosides in length. In some embodiments, the oligonucleotide is at least 9 nucleosides in length. In some embodiments, the oligonucleotide is at least 10 nucleosides in length. In some embodiments, the oligonucleotide is at least 11 nucleosides in length. In some embodiments, the oligonucleotide is at least 12 nucleosides in length. In some embodiments, the oligonucleotide is at least 15 nucleosides in length. In some embodiments, the oligonucleotide is at least 15 nucleosides in length. In some embodiments, the oligonucleotide is at least 16 nucleosides in length. In some embodiments, the oligonucleotide is at least 17 nucleosides in length. In some embodiments, the oligonucleotide is at least 18 nucleosides in length. In some embodiments, the oligonucleotide is at least 19 nucleosides in length. In some embodiments, the oligonucleotide is at least 20 nucleosides in length. In some embodiments, the oligonucleotide is at least 25 nucleosides in length. In some embodiments, the oligonucleotide is at least 30 nucleosides in length. In some embodiments, the oligonucleotide is a duplex of complementary strands of at least 18 nucleosides in length. In some embodiments, the oligonucleotide is a duplex of complementary strands of at least 21 nucleosides in length. In some embodiments, each nucleoside counted in an oligonucleotide length independently comprises A, T, C, G, or U, or optionally substituted A, T, C, G, or U, or an optionally substituted tautomer of A, T, C, G or U.

Oligonucleotide type: As used herein, the phrase “oligonucleotide type” is used to define an oligonucleotide that has a particular base sequence, pattern of backbone linkages (i.e., pattern of internucleotidic linkage types, for example, phosphate, phosphorothioate, phosphorothioate triester, etc.), pattern of backbone chiral centers [i.e., pattern of linkage phosphorus stereochemistry (Rp/Sp)], and pattern of backbone phosphorus modifications (e.g., pattern of “—XLR¹” groups in Formula I as defined herein). In some embodiments, oligonucleotides of a common designated “type” are structurally identical to one another.

One of skill in the art will appreciate that synthetic methods of the present disclosure provide for a degree of control during the synthesis of an oligonucleotide strand such that each nucleotide unit of the oligonucleotide strand can be designed and/or selected in advance to have a particular stereochemistry at the linkage phosphorus and/or a particular modification at the linkage phosphorus, and/or a particular base, and/or a particular sugar. In some embodiments, an oligonucleotide strand is designed and/or selected in advance to have a particular combination of stereocenters at the linkage phosphorus. In some embodiments, an oligonucleotide strand is designed and/or determined to have a particular combination of modifications at the linkage phosphorus. In some embodiments, an oligonucleotide strand is designed and/or selected to have a particular combination of bases. In some embodiments, an oligonucleotide strand is designed and/or selected to have a particular combination of one or more of the above structural characteristics. In some embodiments, the present disclosure provides compositions comprising or consisting of a plurality of oligonucleotide molecules (e.g., chirally controlled oligonucleotide compositions). In some embodiments, all such molecules are of the same type (i.e., are structurally identical to one another). In some embodiments, however, provided compositions comprise a plurality of oligonucleotides of different types, typically in pre-determined relative amounts.

Optionally Substituted: As described herein, compounds, e.g., oligonucleotides, of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}, —SC(S)SR^{o}; —(CH₂)₀₋₄SC(O)R^{o}; —(CH₂)₀₋₄C(O)NR^{o}₂; —C(S)NR^{o}₂; —C(S)SR^{o}; —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —Si(R^{o})₃; —OSi(R^{o})₃; —B(R^{o})₂; —OB(R^{o})₂; —OB(OR^{o})₂; —P(R^{o})₂; —P(OR^{o})₂; —P(R^{o})(OR^{o}); —OP(R^{o})₂; —OP(OR^{o})₂; —OP(R^{o})(OR^{o}); —P(O)(R^{o})₂; —P(O)(OR^{o})₂; —OP(O)(R^{o})₂; —OP(O)(OR^{o})₂; —OP(O)(OR^{o})(SR^{o}); —SP(O)(R^{o})₂; —SP(O)(OR^{o})₂; —N(R^{o})P(O)(R^{o})₂; —N(R^{o})P(O)(OR^{o})₂; —P(R^{o})₂[B(R^{o})₃]; —P(OR^{o})₂[B(R^{o})₃]; —OP(R^{o})₂[B(R^{o})₃]; —OP(OR^{o})₂[B(R^{o})₃]; —(C₁₋₄ straight or branched alkylene)O—N(R^{o})₂; or —(C₁₋₄ straight or branched alkylene)C(O)O—N(R^{o})₂, wherein each R^{o} may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{o}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{o} (or the ring formed by taking two independent occurrences of R^{o} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{o} include ═O and ═S.

Suitable divalent substituents, e.g., on a suitable carbon atom, are independently the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R* are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen are independently —R†, —NR†₂, —C(O)R†, —C(O)OR†, —C(O)C(O)R†, —C(O)CH₂C(O)R†, —S(O)₂R†, —S(O)₂NR†₂, —C(S)NR†₂, —C(NH)NR†₂, or —N(R†)S(O)₂R†; wherein each R† is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R†, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of RT are independently halogen, —R^{•}, -(haloR^{•}), —OH, —OR^{•}, —O(haloR′), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Oral: The phrases “oral administration” and “administered orally” as used herein have their art-understood meaning referring to administration by mouth of a compound or composition.

P-modification: as used herein, the term “P-modification” refers to any modification at the linkage phosphorus other than a stereochemical modification. In some embodiments, a P-modification comprises addition, substitution, or removal of a pendant moiety covalently attached to a linkage phosphorus. In some embodiments, the “P-modification” is —X-L-R¹ wherein each of X, L and R¹ is independently as defined and described in the present disclosure.

Parenteral: The phrases “parenteral administration” and “administered parenterally” as used herein have their art-understood meaning referring to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, e.g., an oligonucleotide, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups, for example, an oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations. In some embodiments, each phosphorothioate and phosphate group independently exists in its salt form (e.g., if sodium salt, —O—P(O)(SNa)—O— and —O—P(O)(ONa)—O—, respectively). In some embodiments, each phosphorothioate and phosphate internucleotidic linkage independently exists in its salt form (e.g., if sodium salt, —O—P(O)(SNa)—O— and —O—P(O)(ONa)—O—, respectively). In some embodiments, a pharmaceutically acceptable salt is a sodium salt of an oligonucleotide. In some embodiments, a pharmaceutically acceptable salt is a sodium salt of an oligonucleotide, wherein each acidic phosphate and modified phosphate group (e.g., phosphorothioate, phosphate, etc.), if any, exists as a salt form (all sodium salt).

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. June 2012, the entirety of Chapter 2 is incorporated herein by reference. Suitable amino-protecting groups include but are not limited to described herein and/or in: WO 2018/022473, WO 2018/098264, WO 2018/223056, WO 2018/223073, WO 2018/223081, WO 2018/237194, WO 2019/032607, WO 2019/055951, and/or WO 2019/075357, the description of the protecting groups of each of which is independently incorporated herein by reference.

Subject: As used herein, the term “subject” or “test subject” refers to any organism to which a provided compound (e.g., a provided oligonucleotide) or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. A base sequence which is substantially complementary to a second sequence is not identical to the second sequence, but is mostly or nearly identical to the second sequence. In addition, one of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Sugar: The term “sugar” refers to a monosaccharide or polysaccharide in closed and/or open form. In some embodiments, sugars are monosaccharides. In some embodiments, sugars are polysaccharides. Sugars include, but are not limited to, ribose, deoxyribose, pentofuranose, pentopyranose, and hexopyranose moieties. As used herein, the term “sugar” also encompasses structural analogs used in lieu of conventional sugar molecules, such as glycol, polymer of which forms the backbone of the nucleic acid analog, glycol nucleic acid (“GNA”), etc. As used herein, the term “sugar” also encompasses structural analogs used in lieu of natural or naturally-occurring nucleotides, such as modified sugars and nucleotide sugars. In some embodiments, a sugar is a RNA or DNA sugar (ribose or deoxyribose). In some embodiments, a sugar is a modified ribose or deoxyribose sugar, e.g., 2′-modified, 5′-modified, etc. As described herein, in some embodiments, when used in oligonucleotides and/or nucleic acids, modified sugars may provide one or more desired properties, activities, etc. In some embodiments, a sugar is optionally substituted ribose or deoxyribose. In some embodiments, a “sugar” refers to a sugar unit in an oligonucleotide or a nucleic acid.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a provided compound, e.g., a provided oligonucleotide.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated,” as used herein, means that a moiety has one or more units of unsaturation.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild type genes and polypeptides often exist in multiple different forms (e.g., alleles).

For purposes of this disclosure, the chemical elements are identified in accordance with the Periodic Table of the Elements, CAS version, Handbook of Chemistry and Physics, 67^{th} Ed., 1986-87, inside cover.

As those skilled in the art will appreciate, methods and compositions described herein relating to provided compounds (e.g., oligonucleotides) also apply to pharmaceutically acceptable salts of such compounds.

### Description of Certain Embodiments

Oligonucleotides are useful tools for a wide variety of applications. For example, HTT oligonucleotides are useful in therapeutic, diagnostic, and research applications, including the treatment of a variety of HTT-related conditions, disorders, and diseases, including Huntington's Disease. The use of naturally occurring nucleic acids (e.g., unmodified DNA or RNA) is limited, for example, by their susceptibility to endo- and exo-nucleases. As such, various synthetic counterparts have been developed to circumvent these shortcomings and/or to further improve various properties and activities. These include synthetic oligonucleotides that contain chemical modifications, e.g., base modifications, sugar modifications, backbone modifications, etc., which, among other things, render these molecules less susceptible to degradation and improve other properties and/or activities. From a structural point of view, modifications to internucleotidic linkages can introduce chirality, and certain properties may be affected by configurations of linkage phosphorus atoms of oligonucleotides. For example, binding affinity, sequence specific binding to complementary RNA, stability to nucleases, cleavage of target HTT nucleic acids, delivery, pharmacokinetics, etc. can be affected by, inter alia, chirality of backbone linkage phosphorus atoms. Among other things, the present disclosure provides technologies for controlling and/or utilizing various structural elements, e.g., sugar modifications and patterns thereof, nucleobase modifications and patterns thereof, modified internucleotidic linkages and patterns thereof, linkage phosphorus stereochemistry and patterns thereof, additional chemical moieties (moieties that are not typically in an oligonucleotide chain) and patterns thereof, etc., and various combinations of one or more or all of such structural elements, in oligonucleotides.

In some embodiments, provided oligonucleotides are oligonucleotides targeting HTT, and can reduce levels of mutant HTT transcripts and/or one or more products encoded thereby. Such oligonucleotides are particularly useful for preventing and/or treating HTT-related conditions, disorders and/or diseases, including Huntington's Disease.

In some embodiments, an HTT oligonucleotide comprises a sequence that is completely or substantially identical to or is completely or substantially complementary to 10, 11, 12, 13, 14, 15, 16, 17, 18, 19, 20, 21, 22, 23, 24, 25, typically 15, 16, 17, 18, 19, 20, 21, 22, 23, 24, 25 or more, contiguous bases of an HTT genomic sequence or a transcript therefrom (e.g., pre-mRNA, mRNA, etc.). Those skilled in the art will appreciate that a “HTT oligonucleotide” may have a nucleotide sequence that is identical (or substantially identical) or complementary (or substantially complementary) to an HTT base sequence (e.g., a genomic sequence, a transcript sequence, a mRNA sequence, etc.) or a portion thereof.

In some embodiments, the present disclosure provides an HTT oligonucleotide as disclosed herein, e.g., in a Table, or an HTT oligonucleotide which has a base sequence comprising at least 10 contiguous bases of an oligonucleotide disclosed herein.

In some embodiments, the present disclosure provides an HTT oligonucleotide having a base sequence disclosed herein, e.g., in a Table, or a portion thereof comprising at least 10 contiguous bases, wherein the HTT oligonucleotide is stereorandom or not chirally controlled.

In some embodiments, internucleotidic linkages of an oligonucleotide comprise or consist of 1-5, 1-10, 1-15, 1-20, 1-25, 1-30, 1-40, 1-50, or 1, 2, 3, 4, 5, 6, 7, 8, 9, 10, 11, 12, 13, 14, 15, 16, 17, 18, 19, 20 or more chirally controlled internucleotidic linkages. In some embodiments, an oligonucleotide composition of the present disclosure comprises oligonucleotides of the same constitution, wherein one or more internucleotidic linkages are chirally controlled and one or more internucleotidic linkages are stereorandom (not chirally controlled). In some embodiments, the present disclosure provides an HTT oligonucleotide composition wherein the HTT oligonucleotides comprise at least one chirally controlled internucleotidic linkage. In some embodiments, the present disclosure provides an HTT oligonucleotide composition wherein the HTT oligonucleotides are stereorandom or not chirally controlled. In some embodiments, in an HTT oligonucleotide, at least one internucleotidic linkage is stereorandom and at least one internucleotidic linkage is chirally controlled.

In some embodiments, internucleotidic linkages of an oligonucleotide comprise or consist of one or more negatively charged internucleotidic linkages (e.g., phosphorothioate internucleotidic linkages, natural phosphate linkages, etc.). In some embodiments, internucleotidic linkages of an oligonucleotide comprise or consist of one or more negatively charged chiral internucleotidic linkages (e.g., phosphorothioate internucleotidic linkages). In some embodiments, internucleotidic linkages of an oligonucleotide comprise or consist of one or more non-negatively charged internucleotidic linkages. In some embodiments, internucleotidic linkages of an oligonucleotide comprise or consist of one or more neutral chiral internucleotidic linkages. In some embodiments, the present disclosure pertains to an HTT oligonucleotide which comprises at least one neutral or non-negatively charged internucleotidic linkage as described in the present disclosure.

### HTT

In some embodiments, HTT refers to a gene or a gene product thereof (including but not limited to, a nucleic acid, including but not limited to a DNA or RNA, or a wild-type or mutant protein encoded thereby), from any species, and which may be also known as: HTT, HD, IT15, huntingtin, Huntingtin, or LOMARS; External IDs: OMIM: 613004, MGI: 96067, HomoloGene: 1593, GeneCards: HTT; Species: Human: Entrez: 3064; Ensembl: ENSG00000197386; UniProt: P42858; RefSeq (mRNA): NM_002111; RefSeq (protein): NP_002102; Location (UCSC): Chr 4: 3.04-3.24 Mb; Species: Mouse: Entrez: 15194; Ensembl: ENSMUSG00000029104; UniProt: P42859; RefSeq (mRNA): NM_010414; RefSeq (protein): NP_034544; Location (UCSC): Chr 5: 34.76-34.91 Mb. Additional HTT sequences, including variants thereof, from human, mouse, rat, monkey, etc., are readily available to those of skill in the art. In some embodiments, HTT is a human or mouse HTT, which is wild-type or mutant.

In some embodiments, an HTT protein is unmodified or modified. In some embodiments, an HTT protein has any one or more modifications of: 9 N6-acetyllysine; 176 N6-acetyllysine; 234 N6-acetyllysine; 343 N6-acetyllysine; 411 Phosphoserine; 417 Phosphoserine; 419 Phosphoserine; 432 Phosphoserine; 442 N6-acetyllysine; 640 Phosphoserine; 643 Phosphoserine; 1179 Phosphoserine; 1199 Phosphoserine; 1870 Phosphoserine; or 1874 Phosphoserine.

Without wishing to be bound by any particular theory, the present disclosure notes that a mutation (e.g., a CAG repeat expansion) in HTT is reportedly a key factor in diseases and disorders such as Huntington's Disease.

In some embodiments, a mutant HTT is designated mHTT, muHTT, m HTT, mu HTT, MU HTT, or the like, wherein m or mu indicate mutant. In some embodiments, a wild type HTT is designated wild-type HTT, wtHTT, wt HTT, WT HTT, WTHTT, or the like, wherein wt indicates wild-type. In some embodiments, a mutant HTT comprises an expanded CAG repeat region (e.g., 36-121, 36-250, 37-121, 40-121, repeats or longer). In some embodiments, a mutant HTT comprises a mutant allele of one or more SNP (the allele on the same DNA strand or chromosome as the expanded CAG repeats). In some embodiments, a mutant HTT comprises both an expanded CAG repeat region and a mutant allele of a particular SNP on the same chromosomal strand.

In some embodiments, a human HTT is designated hHTT. In some embodiments, a mutant HTT is designated mHTT. In some embodiments, when a mouse is utilized, a mouse HTT may be referred to as mHTT as those skilled in the art will appreciate.

In some embodiments, an HTT oligonucleotide is complementary to a portion of an HTT nucleic acid sequence, e.g., an HTT gene sequence, an HTT mRNA sequence, etc. In some embodiments, the base sequence of such a portion is characteristic of HTT in that no other genomic or transcript sequences have the same sequence as the portion. In some embodiments, a portion of a gene that is complimentary to an oligonucleotide is referred to as the target sequence of the oligonucleotide.

In some embodiments, an HTT gene sequence (or a portion thereof, e.g., complementary to an HTT oligonucleotide) is an HTT gene sequence (or a portion thereof) known in the art or reported in the literature. Certain nucleotide and amino acid sequences of a human HTT can be found in public sources, for example, one or more publicly available databases, e.g., GenBank, UniProt, OMEVI, etc. Those skilled in the art will appreciate that, for example, where a described nucleic acid sequence may be or include a genomic sequence, transcripts, splicing products, and/or encoded proteins, etc., may readily be appreciated from such genomic sequence.

In some embodiments, an HTT gene (or a portion thereof with a sequence complementary to an HTT oligonucleotide) includes a single nucleotide polymorphism or SNP. Numerous HTT SNPs have been reported and may be found at, for example, NCBI dbSNP (see, e.g., www.ncbi.nlm.nih.gov/snp). Non-limiting examples of SNPs within the HTT gene may be found at, NCBI dbSNP Accession, and include, for example, those described herein. In some embodiments, an HTT oligonucleotide targets a SNP allele which is on the same chromosome as (e.g., in phase with) the CAG repeat expansion and not present on the wild-type allele (which does not comprise the CAG repeat expansion).

Huntinton's disease (HD) is a neurodegenerative disorder reportedly caused by a mutation of the HTT (huntingtin) gene. Alteration of this widely expressed single gene reportedly results in a progressive, neurodegenerative disorder with a large number of characteristic symptoms. In some embodiments, a HD-related mutation is an expansion of a CAG repeat region in the HTT gene, wherein a larger expansion reportedly results in greater severity of the disease and an earlier age of onset. The mutation reportedly results in a variety of motor, emotional and cognitive symptoms, and results in the formation of huntingtin aggregates in brain.

The CAG expansion reportedly results in the expansion of a poly-glutamine tract in the huntingtin protein, a 350 kDa protein (Huntington Disease Collaborative Research Group, 1993. Cell. 72:971-83). The normal and expanded HD allele sizes have reportedly been found to be, e.g., CAG 6-37 and CAG 35-121 repeats or longer, respectively. Longer repeat sequences are reportedly associated with earlier disease onset. The absence of an HD phenotype in individuals deleted for one copy of huntingtin, or increased severity of disease in those homozygous for the expansion reportedly suggests that the mutation does not result in a loss of function (Trottier et al., 1995, Nature Med., 10:104-110). Transcriptional deregulation and loss of function of transcriptional coactivator proteins have reportedly been implicated in HD pathogenesis. Mutant huntingtin has reportedly been shown specifically to disrupt activator-dependent transcription in the early stages of HD pathogenesis (Dunah et al., 2002. Science 296:2238-2243).

In one report gene profiling of human blood identified 322 mRNAs that show significantly altered expression in HD blood samples as compared to normal or presymptomatic individuals. Expression of marker genes was similarly substantially altered in post-mortem brain samples from HD caudate, suggesting that upregulation of genes in blood samples reflects disease mechanisms found in brain. Monitoring of gene expression may provide a sensitive and quantitative method to monitor disease progression, especially in the early stages of disease in both animal models and human patients (Borovecki et al., 2005, Proc. Natl. Acad. Sci. USA 102:11023-11028).

Huntington's disease has been reported to be an autosomal dominant disorder, with an onset generally in mid-life, although cases of onset from childhood to over 70 years of age have been documented. An earlier age of onset is reportedly associated with paternal inheritance, with 70% of juvenile cases being inherited through the father.

In some embodiments, symptoms of Huntington's Disease have an emotional, motor and cognitive component. One symptom, chorea is a characteristic feature of the motor disorder and is defined as excessive spontaneous movements which are irregularly timed, randomly distributed and abrupt. It can vary from being barely perceptible to severe. Other frequently observed symptoms or abnormalities include dystonia, rigidity, bradykinesia, ocularmotor dysfunction, tremor, etc. Voluntary movement disorders as symptoms include fine motor incoordination, dysathria, and dysphagia. Emotional disorders or symptoms commonly include depression and irritability, and cognitive component comprises subcortical dementia (Mangiarini et al. 1996. Cell 87:493-506). It is reported that changes in HD brains are widespread and include neuronal loss and gliosis, particularly in the cortex and striatum (Vonsattel and DiFiglia. 1998. J. Neuropathol. Exp. Neurol. 57:369-384).

Certain information related to HTT and HTT-related conditions, disorders or diseases has been reported in, for example: Kremer et al. 1994. N. E. J. Med. 330: 1401; Kordasiewicz et al. 2012 Neuron 74: 1031-1044; Carroll et al. 2011 Mol. Ther. 19: 2178-2185; Warby et al. 2009 Am. J. Hum. Genet. 84: 351-366; Pfister et al. 2009 Current Biol. 19: 774-778; Kay et al. 2015 Mol. Ther. 23: 1759-1771; Kay et al. 2014 Clin. Genet. 86: 29-36; Lee et al. 2015. Am. J. Hum. Genet. 97: 435-444; Skotte et al. 2014. PLOS ONE 9: e107434; Southwell et al. 2014. Mol. Ther. 22: 2093-2106; Australian Pat. Publications AU2017276286 and AU2007210038; European Pat. Publications EP3277814 and EP3210633; International Pat. Publication WO2018145009; and US Pat. Publication US20180273945.

In some embodiments, an HTT oligonucleotide capable of decreasing the level, activity and/or expression of an HTT gene is useful in a method of preventing or treating an HTT-related condition, disorder or disease, e.g., Huntington's Disease, and/or delaying the onset of and/or the severity of one or more symptoms of Huntington's Disease.

In some embodiments, the present disclosure provides methods for preventing or treating an HTT-related condition, disorder or disease, by administering to a subject suffering from or susceptible to such a condition, disorder or disease a therapeutically effective amount of a provided HTT oligonucleotide or a composition thereof. In some embodiments, a composition is a chirally controlled oligonucleotide composition.

### HTT Oligonucleotides

Among other things, the present disclosure provides oligonucleotides of various designs, which may comprises various nucleobases and patterns thereof, sugars and patterns thereof, internucleotidic linkages and patterns thereof, and/or additional chemical moieties and patterns thereof as described in the present disclosure. In some embodiments, provided oligonucleotides are HTT oligonucleotides. In some embodiments, provided HTT oligonucleotides can direct a decrease in the expression, level and/or activity of an HTT gene and/or one or more of its products (e.g., transcripts, mRNA, proteins, etc.). In some embodiments, provided HTT oligonucleotides can direct a decrease in the expression, level and/or activity of an HTT gene and/or one or more of its products in any cell of a subject or patient. In some embodiments, a cell is a any cell that normally expresses HTT or produces HTT protein. In some embodiments, provided HTT oligonucleotides can direct a decrease in the expression, level and/or activity of an HTT target gene or a gene product and has a base sequence which consists of, comprises, or comprises a portion (e.g., a span of 10, 11, 12, 13, 14, 15, 16, 17, 18, 19 or more contiguous bases) of the base sequence of an HTT oligonucleotide disclosed herein, and the oligonucleotide comprises at least one non-naturally-occurring modification of a base, sugar and/or internucleotidic linkage.

In some embodiments, an HTT oligonucleotide comprises one or more carbohydrate moieties. In some embodiments, an HTT oligonucleotide comprises one or more lipid moieties. In some embodiments, an HTT oligonucleotide comprises one or more targeting moieties. Non-limiting examples of such additional chemical moieties which can be conjugated to an oligonucleotide chain are described herein.

In some embodiments, provided oligonucleotides can direct a decrease in the expression, level and/or activity of a target gene, e.g., an HTT target gene, or a product thereof. In some embodiments, provided oligonucleotides can direct a decrease in the expression, level and/or activity of an HTT target gene or a product thereof via RNase H-mediated knockdown. In some embodiments, provided oligonucleotides can direct a decrease in the expression, level and/or activity of an HTT target gene or a product thereof by sterically blocking translation after binding to an HTT target gene mRNA, and/or by altering or interfering with mRNA splicing. Regardless, however, the present disclosure is not limited to any particular mechanism. In some embodiments, the present disclosure provides oligonucleotides, compositions, methods, etc., capable of operating via double-stranded RNA interference, single-stranded RNA interference, RNase H-mediated knock-down, steric hindrance of translation, or a combination of two or more such mechanisms.

In some embodiments, HTT oligonucleotides are antisense oligonucleotides (ASOs), in that they are oligonucleotides which have a base sequence which is antisense (e.g., complementary) to a target HTT sequence. In some embodiments, HTT oligonucleotides are double-stranded siRNAs. In some embodiments, HTT oligonucleotides are single-stranded siRNAs. Provided oligonucleotides and compositions thereof may be utilized for many purposes. For example, provided HTT oligonucleotides can be co-administered or be used as part of a treatment regimen along with one or more treatment for Huntington's Disease or a symptom thereof, including but not limited to: aptamers, lncRNAs, lncRNA inhibitors, antibodies, peptides, small molecules, other oligonucleotides to HTT or other targets, and/or other agents capable of inhibiting the expression of an HTT transcript, reducing the level and/or activity of an HTT gene product, and/or inhibiting the expression of a gene or reducing a gene product thereof which increases the expression, activity and/or level of an HTT transcript or an HTT gene product, or a gene or gene product which is associated with an HTT-related disorder.

In some embodiments, an oligonucleotide, e.g., an HTT oligonucleotide, comprises a structural element or a portion thereof described herein, e.g., in a Table. In some embodiments, an oligonucleotide, e.g., an HTT oligonucleotide, comprises a base sequence (or a portion thereof), a chemical modification or a pattern of chemical modifications (or a portion thereof), and/or a format or a portion thereof described herein. In some embodiments, an oligonucleotide, e.g., an HTT oligonucleotide, comprises the base sequence (or a portion thereof), pattern of chemical modifications (or a portion thereof), and/or a format of an oligonucleotide disclosed herein, e.g., in Table 1 or in the Figures, or otherwise disclosed herein. In some embodiments, such oligonucleotides, e.g., HTT oligonucleotides reduce expression, level and/or activity of a gene, e.g., an HTT gene, or a gene product thereof.

Among other things, provided oligonucleotides may hybridize to their target HTT nucleic acids (e.g., pre-mRNA, mature mRNA, etc.). For example, in some embodiments, an HTT oligonucleotide can hybridize to an HTT nucleic acid derived from a DNA strand (either strand of the HTT gene). In some embodiments, an HTT oligonucleotide can hybridize to an HTT transcript. In some embodiments, an HTT oligonucleotide can hybridize to an HTT nucleic acid in any stage of RNA processing, including but not limited to a pre-mRNA or a mature mRNA. In some embodiments, an HTT oligonucleotide can hybridize to any element of an HTT nucleic acid or its complement, including but not limited to: a promoter region, an enhancer region, a transcriptional stop region, a translational start signal, a translation stop signal, a coding region, a non-coding region, an exon, an intron, an intron/exon or exon/intron junction, the 5′ UTR, or the 3′ UTR.

In some embodiments, an oligonucleotide hydridizes to two or more variants of transcripts derived from a sense strand. In some embodiments, an HTT oligonucleotide hybridizes to two or more variants of HTT derived from the sense strand. In some embodiments, an HTT oligonucleotide hybridizes to all variants of HTT derived from the sense strand. In some embodiments, an HTT oligonucleotide hybridizes to two or more variants of HTT derived from the antisense strand. In some embodiments, an HTT oligonucleotide hybridizes to all variants of HTT derived from the antisense strand.

In some embodiments, an HTT target of an HTT oligonucleotide is an HTT RNA which is not a mRNA.

In some embodiments, HTT oligonucleotides contain increased levels of one or more isotopes. In some embodiments, provided oligonucleotides are labeled, e.g., by one or more isotopes of one or more elements, e.g., hydrogen, carbon, nitrogen, etc. In some embodiments, provided oligonucleotides in provided compositions, e.g., oligonucleotides of a plurality of a composition, comprise base modifications, sugar modifications, and/or internucleotidic linkage modifications, wherein the oligonucleotides contain an enriched level of deuterium. In some embodiments, provided oligonucleotides are labeled with deuterium (replacing —¹H with —²H) at one or more positions. In some embodiments, one or more ¹H of an oligonucleotide chain or any moiety conjugated to the oligonucleotide chain (e.g., a targeting moiety, etc.) is substituted with ²H. Such oligonucleotides can be used in compositions and methods described herein.

In some embodiments, the present disclosure provides an oligonucleotide composition comprising a plurality of oligonucleotides which:
1) have a common base sequence complementary to a target sequence (e.g., an HTT target sequence) in a transcript; and
2) comprise one or more modified sugar moieties and/or modified internucleotidic linkages.

In some embodiments, oligonucleotides, e.g., HTT oligonucleotides, having a common base sequence may have the same pattern of nucleoside modifications, e.g., sugar modifications, base modifications, etc. In some embodiments, a pattern of nucleoside modifications may be represented by a combination of locations and modifications. In some embodiments, a pattern of backbone linkages comprises locations and types (e.g., phosphate, phosphorothioate, substituted phosphorothioate, etc.) of each internucleotidic linkage.

In some embodiments, a modified internucleotidic linkage has a structure of Formula I. In some embodiments, a modified internucleotidic linkage has a structure of Formula I-a. In some embodiments, an internucleotidic linkage has the structure of Formula I, I-a, I-b, I-c, I-n-1, I-n-2, I-n-3, I-n-4, II, II-a-1, II-a-2, II-b-1, II-b-2, II-c-1, II-c-2, II-d-1, or II-d-2, or a salt form thereof.

In some embodiments, a HTT oligonucleotide comprises one or more internucleotidic linkage, each of which independently has the structure of Formula I, I-a, I-b, I-c, I-n-1, I-n-2, I-n-3, I-n-4, II, II-a-1, II-a-2, II-b-1, II-b-2, II-c-1, II-c-2, II-d-1, or II-d-2.

In some embodiments, oligonucleotides of a plurality, e.g., in provided compositions, are of the same oligonucleotide type. In some embodiments, oligonucleotides of an oligonucleotide type have a common pattern of sugar modifications. In some embodiments, oligonucleotides of an oligonucleotide type have a common pattern of base modifications. In some embodiments, oligonucleotides of an oligonucleotide type have a common pattern of nucleoside modifications. In some embodiments, oligonucleotides of an oligonucleotide type have the same constitution. In some embodiments, oligonucleotides of an oligonucleotide type are identical. In some embodiments, oligonucleotides of a plurality are identical. In some embodiments, oligonucleotides of a plurality share the same constitution.

In some embodiments, as exemplified herein, oligonucleotides, e.g., HTT oligonucleotides, are chiral controlled, comprising one or more chirally controlled internucleotidic linkages. In some embodiments, provided oligonucleotides are stereochemically pure. In some embodiments, provided oligonucleotides are substantially separated from other stereoisomers.

In some embodiments, oligonucleotides, e.g., HTT oligonucleotides, comprise one or more modified nucleobases, one or more modified sugars, and/or one or more modified internucleotidic linkages.

In some embodiments, oligonucleotides, e.g., HTT oligonucleotides, comprise one or more modified sugars. In some embodiments, oligonucleotides of the present disclosure comprise one or more modified nucleobases. Various modifications can be introduced to a sugar and/or nucleobase in accordance with the present disclosure. For example, in some embodiments, a modification is a modification described in U.S. Pat. No. 9,006,198. In some embodiments, a modification is a modification described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, or WO 2018/098264, the sugar, base, and internucleotidic linkage modifications of each of which are independently incorporated herein by reference.

As used in the present disclosure, in some embodiments, one or more is one. In some embodiments, one or more is two. In some embodiments, one or more is three. In some embodiments, one or more is four. In some embodiments, one or more is five. In some embodiments, one or more is six. In some embodiments, one or more is seven. In some embodiments, one or more is eight. In some embodiments, one or more is nine. In some embodiments, one or more is ten. In some embodiments, one or more is at least one. In some embodiments, one or more is at least two. In some embodiments, one or more is at least three. In some embodiments, one or more is at least four. In some embodiments, one or more is at least five. In some embodiments, one or more is at least six. In some embodiments, one or more is at least seven. In some embodiments, one or more is at least eight. In some embodiments, one or more is at least nine. In some embodiments, one or more is at least ten.

In some embodiments, an HTT oligonucleotide is or comprises an HTT oligonucleotide described in a Table or Figure.

As demonstrated in the present disclosure, in some embodiments, a provided oligonucleotide (e.g., an HTT oligonucleotide) is characterized in that, when it is contacted with the transcript in a knockdown system, knockdown of its target (e.g., an HTT transcript for an HTT oligonucleotide, a mutant HTT transcript comprising expanded CAG repeats, etc.) is improved relative to that observed under reference conditions (e.g., selected from the group consisting of absence of the composition, presence of a reference composition, and combinations thereof). In some embodiments, knockdown is increased 10%, 20%, 30%, 40%, 50%, 60%, 70%, 80%, 90%, 100%, or 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, 1000 fold or more.

In some embodiments, oligonucleotides are provided as salt forms. In some embodiments, oligonucleotides are provided as salts comprising negatively-charged internucleotidic linkages (e.g., phosphorothioate internucleotidic linkages, natural phosphate linkages, etc.) existing as their salt forms. In some embodiments, oligonucleotides are provided as pharmaceutically acceptable salts. In some embodiments, oligonucleotides are provided as metal salts. In some embodiments, oligonucleotides are provided as sodium salts. In some embodiments, oligonucleotides are provided as metal salts, e.g., sodium salts, wherein each negatively-charged internucleotidic linkage is independently in a salt form (e.g., for sodium salts, —O—P(O)(SNa)—O— for a phosphorothioate internucleotidic linkage, —O—P(O)(ONa)—O— for a natural phosphate linkage, etc.).

In some embodiments, a HTT oligonucleotide or a HTT oligonucleotide composition is chirally controlled (e.g., stereopure).

In some embodiments, a HTT oligonucleotide or a HTT oligonucleotide is stereorandom.

In some embodiments, a HTT oligonucleotide targets HTT SNP rs362272, rs362273, rs362273, rs362307, rs362331, or rs363099.

In some embodiments, a HTT oligonucleotide targets SNP rs362272 and has a base sequence which comprises: ACATAGAGGACGCCGTGCAG, AGAGGACGCCGTGCAGGGCT, ATAGAGGACGCCGTGCAGGG, CACATAGAGGACGCCGTGCA, CATAGAGGACGCCGTGCAGG, GCACATAGAGGACGCCGTGC, or TAGAGGACGCCGTGCAGGGC, wherein each T can be independently substituted with U or vice versa.

In some embodiments, a HTT oligonucleotide targets SNP rs362273 and has a base sequence which comprises: AGCTGCTGCTACAGATCAAC, AGCTGCTGCTGCAGATCAAC, GGTTGATCTGTAGCAGCAGCT, GTTGATCTGTAGCAGCAGCT, GTTGATCTGTAGCAGCAGCT, or TTGATCTGTAGCAGCAGCT, wherein each T can be independently substituted with U or vice versa.

In some embodiments, a HTT oligonucleotide targets SNP rs362273 and has a base sequence which comprises: GTTGATCTGTAGCAGCAGCT, wherein each T can be independently substituted with U or vice versa.

In some embodiments, a HTT oligonucleotide targets SNP rs362307 and has a base sequence which comprises: CACAAGGGCACAGACTTCCA, GGCACAAGGGCACAGAC, GGCACAAGGGCACAGACT, GGCACAAGGGCACAGACTT, or GGCACAAGGGCACAGACTTC, wherein each T can be independently substituted with U or vice versa.

In some embodiments, a HTT oligonucleotide targets SNP rs362331 and has a base sequence which comprises: AGTGCACACAGTAGATGAGG, GTGCACACAGTAGATGAGGG, or TGCACACAGTAGATGAGGGA, wherein each T can be independently substituted with U or vice versa.

In some embodiments, a HTT oligonucleotide targets SNP rs363099 and has a base sequence which comprises: AAGGCTGAGCGGAGAAACCC, AGGCTGAGCGGAGAAACCCT, CAAGGCTGAGCGGAGAAACC, CTGAGCGGAGAAACCCTCCA, GCTGAGCGGAGAAACCCTCC, GGCTGAGCGGAGAAACCCTC, or TGAGCGGAGAAACCCTCCAA, wherein each T can be independently substituted with U or vice versa.

In some embodiments, a HTT oligonucleotide targets SNP rs362272 and has a base sequence which is: ACATAGAGGACGCCGTGCAG, AGAGGACGCCGTGCAGGGCT, ATAGAGGACGCCGTGCAGGG, CACATAGAGGACGCCGTGCA, CATAGAGGACGCCGTGCAGG, GCACATAGAGGACGCCGTGC, or TAGAGGACGCCGTGCAGGGC, wherein each T can be independently substituted with U or vice versa.

In some embodiments, a HTT oligonucleotide targets SNP rs362273 and has a base sequence which is: AGCTGCTGCTACAGATCAAC, AGCTGCTGCTGCAGATCAAC, GGTTGATCTGTAGCAGCAGCT, GTTGATCTGTAGCAGCAGCT, GTTGATCTGTAGCAGCAGCT, or TTGATCTGTAGCAGCAGCT, wherein each T can be independently substituted with U or vice versa.

In some embodiments, a HTT oligonucleotide targets SNP rs362273 and has a base sequence which is: GTTGATCTGTAGCAGCAGCT, wherein each T can be independently substituted with U or vice versa.

In some embodiments, a HTT oligonucleotide targets SNP rs362307 and has a base sequence which is: CACAAGGGCACAGACTTCCA, GGCACAAGGGCACAGAC, GGCACAAGGGCACAGACT, GGCACAAGGGCACAGACTT, or GGCACAAGGGCACAGACTTC, wherein each T can be independently substituted with U or vice versa.

In some embodiments, a HTT oligonucleotide targets SNP rs362331 and has a base sequence which is: AGTGCACACAGTAGATGAGG, GTGCACACAGTAGATGAGGG, or TGCACACAGTAGATGAGGGA, wherein each T can be independently substituted with U or vice versa.

In some embodiments, a HTT oligonucleotide targets SNP rs363099 and has a base sequence which is: AAGGCTGAGCGGAGAAACCC, AGGCTGAGCGGAGAAACCCT, CAAGGCTGAGCGGAGAAACC, CTGAGCGGAGAAACCCTCCA, GCTGAGCGGAGAAACCCTCC, GGCTGAGCGGAGAAACCCTC, or TGAGCGGAGAAACCCTCCAA, wherein each T can be independently substituted with U or vice versa.

In some embodiments, a HTT oligonucleotide targets SNP rs362272 and has a base sequence which comprises at least 15 contiguous bases, including the position of the SNP, of: ACATAGAGGACGCCGTGCAG, AGAGGACGCCGTGCAGGGCT, ATAGAGGACGCCGTGCAGGG, CACATAGAGGACGCCGTGCA, CATAGAGGACGCCGTGCAGG, GCACATAGAGGACGCCGTGC, or TAGAGGACGCCGTGCAGGGC, wherein each T can be independently substituted with U or vice versa.

In some embodiments, a HTT oligonucleotide targets SNP rs362273 and has a base sequence which comprises at least 15 contiguous bases, including the position of the SNP, of: AGCTGCTGCTACAGATCAAC, AGCTGCTGCTGCAGATCAAC, GGTTGATCTGTAGCAGCAGCT, GTTGATCTGTAGCAGCAGCT, GTTGATCTGTAGCAGCAGCT, or TTGATCTGTAGCAGCAGCT, wherein each T can be independently substituted with U or vice versa.

In some embodiments, a HTT oligonucleotide targets SNP rs362273 and has a base sequence which comprises at least 15 contiguous bases, including the position of the SNP, of: GTTGATCTGTAGCAGCAGCT, wherein each T can be independently substituted with U or vice versa.

In some embodiments, a HTT oligonucleotide targets SNP rs362307 and has a base sequence which comprises at least 15 contiguous bases, including the position of the SNP, of: CACAAGGGCACAGACTTCCA, GGCACAAGGGCACAGAC, GGCACAAGGGCACAGACT, GGCACAAGGGCACAGACTT, or GGCACAAGGGCACAGACTTC, wherein each T can be independently substituted with U or vice versa.

In some embodiments, a HTT oligonucleotide targets SNP rs362331 and has a base sequence which comprises at least 15 contiguous bases, including the position of the SNP, of: AGTGCACACAGTAGATGAGG, GTGCACACAGTAGATGAGGG, or TGCACACAGTAGATGAGGGA, wherein each T can be independently substituted with U or vice versa.

In some embodiments, a HTT oligonucleotide targets SNP rs363099 and has a base sequence which comprises at least 15 contiguous bases, including the position of the SNP, of: AAGGCTGAGCGGAGAAACCC, AGGCTGAGCGGAGAAACCCT, CAAGGCTGAGCGGAGAAACC, CTGAGCGGAGAAACCCTCCA, GCTGAGCGGAGAAACCCTCC, GGCTGAGCGGAGAAACCCTC, or TGAGCGGAGAAACCCTCCAA, wherein each T can be independently substituted with U or vice versa.

In some embodiments, a HTT oligonucleotide targets SNP rs362272 and has a base sequence which comprises at least 10 contiguous bases, including the position of the SNP, of: ACATAGAGGACGCCGTGCAG, AGAGGACGCCGTGCAGGGCT, ATAGAGGACGCCGTGCAGGG, CACATAGAGGACGCCGTGCA, CATAGAGGACGCCGTGCAGG, GCACATAGAGGACGCCGTGC, or TAGAGGACGCCGTGCAGGGC, wherein each T can be independently substituted with U or vice versa.

In some embodiments, a HTT oligonucleotide targets SNP rs362273 and has a base sequence which comprises at least 10 contiguous bases, including the position of the SNP, of: AGCTGCTGCTACAGATCAAC, AGCTGCTGCTGCAGATCAAC, GGTTGATCTGTAGCAGCAGCT, GTTGATCTGTAGCAGCAGCT, GTTGATCTGTAGCAGCAGCT, or TTGATCTGTAGCAGCAGCT, wherein each T can be independently substituted with U or vice versa.

In some embodiments, a HTT oligonucleotide targets SNP rs362273 and has a base sequence which comprises at least 10 contiguous bases, including the position of the SNP, of: GTTGATCTGTAGCAGCAGCT, wherein each T can be independently substituted with U or vice versa.

In some embodiments, a HTT oligonucleotide targets SNP rs362307 and has a base sequence which comprises at least 10 contiguous bases, including the position of the SNP, of: CACAAGGGCACAGACTTCCA, GGCACAAGGGCACAGAC, GGCACAAGGGCACAGACT, GGCACAAGGGCACAGACTT, or GGCACAAGGGCACAGACTTC, wherein each T can be independently substituted with U or vice versa.

In some embodiments, a HTT oligonucleotide targets SNP rs362331 and has a base sequence which comprises at least 10 contiguous bases, including the position of the SNP, of: AGTGCACACAGTAGATGAGG, GTGCACACAGTAGATGAGGG, or TGCACACAGTAGATGAGGGA, wherein each T can be independently substituted with U or vice versa.

In some embodiments, a HTT oligonucleotide targets SNP rs363099 and has a base sequence which comprises at least 10 contiguous bases, including the position of the SNP, of: AAGGCTGAGCGGAGAAACCC, AGGCTGAGCGGAGAAACCCT, CAAGGCTGAGCGGAGAAACC, CTGAGCGGAGAAACCCTCCA, GCTGAGCGGAGAAACCCTCC, GGCTGAGCGGAGAAACCCTC, or TGAGCGGAGAAACCCTCCAA, wherein each T can be independently substituted with U or vice versa.

In some embodiments, a HTT oligonucleotide does not target a SNP, wherein each U can be independently substituted with T and vice versa.

In some embodiments, a HTT oligonucleotide does not target a SNP and is pan-specific, wherein each U can be independently substituted with T and vice versa.

In some embodiments, a HTT oligonucleotide does not target a SNP and is pan-specific, and has a base sequence which comprises, which is, which comprises at least 15 contiguous bases of, or which comprises at least 10 contiguous bases of: ACCGCCATCCCCGCCGTAGC, CCGCCATCCCCGCCGTAGCC, CGCCATCCCCGCCGTAGCCT, CTCAGTAACATTGACACCAC, GCCATCCCCGCCGTAGCCTG, GGCTCTGGGTTGCTGGGTCA, GGTGTCCCTCATGGGCTCTG, or GTTACCGCCATCCCCGCCGT, wherein each U can be independently substituted with T and vice versa.

In some embodiments, a HTT oligonucleotide has a base sequence comprising the sequence of: ACCGCCATCCCCGCCGTAGC, CCGCCATCCCCGCCGTAGCC, CGCCATCCCCGCCGTAGCCT, CTCAGTAACATTGACACCAC, GCCATCCCCGCCGTAGCCTG, GGCTCTGGGTTGCTGGGTCA, GGTGTCCCTCATGGGCTCTG, or GTTACCGCCATCCCCGCCGT, wherein each U can be independently substituted with T and vice versa.

In some embodiments, a HTT oligonucleotide has a base sequence which is the sequence of: ACCGCCATCCCCGCCGTAGC, CCGCCATCCCCGCCGTAGCC, CGCCATCCCCGCCGTAGCCT, CTCAGTAACATTGACACCAC, GCCATCCCCGCCGTAGCCTG, GGCTCTGGGTTGCTGGGTCA, GGTGTCCCTCATGGGCTCTG, or GTTACCGCCATCCCCGCCGT, wherein each U can be independently substituted with T and vice versa.

In some embodiments, a HTT oligonucleotide has a base sequence comprising at least 15 contiguous bases of the sequence of: ACCGCCATCCCCGCCGTAGC, CCGCCATCCCCGCCGTAGCC, CGCCATCCCCGCCGTAGCCT, CTCAGTAACATTGACACCAC, GCCATCCCCGCCGTAGCCTG, GGCTCTGGGTTGCTGGGTCA, GGTGTCCCTCATGGGCTCTG, or GTTACCGCCATCCCCGCCGT, wherein each U can be independently substituted with T and vice versa.

In some embodiments, a HTT oligonucleotide has a base sequence comprising at least 10 contiguous bases of the sequence of: ACCGCCATCCCCGCCGTAGC, CCGCCATCCCCGCCGTAGCC, CGCCATCCCCGCCGTAGCCT, CTCAGTAACATTGACACCAC, GCCATCCCCGCCGTAGCCTG, GGCTCTGGGTTGCTGGGTCA, GGTGTCCCTCATGGGCTCTG, or GTTACCGCCATCCCCGCCGT, wherein each U can be independently substituted with T and vice versa.

In some embodiments, a HTT oligonucleotide is any HTT oligonucleotide disclosed herein, or a salt thereof.

In some embodiments, a HTT oligonucleotide is any of: WV-10786, WV-10787, WV-10790, WV-10791, WV-10806, WV-10810, WV-10811, WV-12282, WV-12283, WV-12284, WV-14914, WV-15078, WV-15080, WV-17782, WV-19824, WV-19825, WV-19840, WV-19841, WV-21178, WV-21179, WV-21180, WV-21181, WV-21267, WV-21271, WV-21274, WV-21403, WV-21404, WV-21405, WV-21406, WV-21409, WV-21410, WV-21412, WV-21447, WV-21448, WV-23689, WV-23690, WV-23691, WV-23692, WV-28152, WV-28153, WV-28154, WV-28155, WV-28156, WV-28157, WV-28158, WV-28159, WV-28160, WV-28161, WV-28162, WV-28163, WV-28164, WV-28165, WV-28166, WV-28167, WV-28168, or WV-9679, or a salt thereof, wherein each U can be independently substituted with T and vice versa.

In some embodiments, a HTT oligonucleotide is any of stereopure (chirally controlled) HTT oligonucleotide which comprises the base sequence of any of: WV-10786, WV-10787, WV-10790, WV-10791, WV-10806, WV-10810, WV-10811, WV-12282, WV-12283, WV-12284, WV-14914, WV-15078, WV-15080, WV-17782, WV-19824, WV-19825, WV-19840, WV-19841, WV-21178, WV-21179, WV-21180, WV-21181, WV-21267, WV-21271, WV-21274, WV-21403, WV-21404, WV-21405, WV-21406, WV-21409, WV-21410, WV-21412, WV-21447, WV-21448, WV-23689, WV-23690, WV-23691, WV-23692, WV-28152, WV-28153, WV-28154, WV-28155, WV-28156, WV-28157, WV-28158, WV-28159, WV-28160, WV-28161, WV-28162, WV-28163, WV-28164, WV-28165, WV-28166, WV-28167, WV-28168, or WV-9679, or a salt thereof, wherein each U can be independently substituted with T and vice versa.

In some embodiments, a HTT oligonucleotide is any of stereopure (chirally controlled) HTT oligonucleotide which has the base sequence of any of: WV-10786, WV-10787, WV-10790, WV-10791, WV-10806, WV-10810, WV-10811, WV-12282, WV-12283, WV-12284, WV-14914, WV-15078, WV-15080, WV-17782, WV-19824, WV-19825, WV-19840, WV-19841, WV-21178, WV-21179, WV-21180, WV-21181, WV-21267, WV-21271, WV-21274, WV-21403, WV-21404, WV-21405, WV-21406, WV-21409, WV-21410, WV-21412, WV-21447, WV-21448, WV-23689, WV-23690, WV-23691, WV-23692, WV-28152, WV-28153, WV-28154, WV-28155, WV-28156, WV-28157, WV-28158, WV-28159, WV-28160, WV-28161, WV-28162, WV-28163, WV-28164, WV-28165, WV-28166, WV-28167, WV-28168, or WV-9679, or a salt thereof, wherein each U can be independently substituted with T and vice versa.

In some embodiments, a HTT oligonucleotide is any of stereopure (chirally controlled) HTT oligonucleotide which has a base sequence comprising at least 15 contiguous bases of the base sequence of any of: WV-10786, WV-10787, WV-10790, WV-10791, WV-10806, WV-10810, WV-10811, WV-12282, WV-12283, WV-12284, WV-14914, WV-15078, WV-15080, WV-17782, WV-19824, WV-19825, WV-19840, WV-19841, WV-21178, WV-21179, WV-21180, WV-21181, WV-21267, WV-21271, WV-21274, WV-21403, WV-21404, WV-21405, WV-21406, WV-21409, WV-21410, WV-21412, WV-21447, WV-21448, WV-23689, WV-23690, WV-23691, WV-23692, WV-28152, WV-28153, WV-28154, WV-28155, WV-28156, WV-28157, WV-28158, WV-28159, WV-28160, WV-28161, WV-28162, WV-28163, WV-28164, WV-28165, WV-28166, WV-28167, WV-28168, or WV-9679, or a salt thereof, wherein each U can be independently substituted with T and vice versa.

In some embodiments, a HTT oligonucleotide is any of stereopure (chirally controlled) HTT oligonucleotide or HTT oligonucleotide which has a base sequence comprising at least 10 contiguous bases of the base sequence of any of: WV-10786, WV-10787, WV-10790, WV-10791, WV-10806, WV-10810, WV-10811, WV-12282, WV-12283, WV-12284, WV-14914, WV-15078, WV-15080, WV-17782, WV-19824, WV-19825, WV-19840, WV-19841, WV-21178, WV-21179, WV-21180, WV-21181, WV-21267, WV-21271, WV-21274, WV-21403, WV-21404, WV-21405, WV-21406, WV-21409, WV-21410, WV-21412, WV-21447, WV-21448, WV-23689, WV-23690, WV-23691, WV-23692, WV-28152, WV-28153, WV-28154, WV-28155, WV-28156, WV-28157, WV-28158, WV-28159, WV-28160, WV-28161, WV-28162, WV-28163, WV-28164, WV-28165, WV-28166, WV-28167, WV-28168, or WV-9679, or a salt thereof, wherein each U can be independently substituted with T and vice versa.

In some embodiments, the present disclosure pertains to: A composition comprising a HTT oligonucleotide and a pharmaceutical carrier.

In some embodiments, the present disclosure pertains to: A method of use of a HTT oligonucleotide in treatment of and/or prevention of Huntington's Disease.

In some embodiments, the present disclosure pertains to: A method of use of a HTT oligonucleotide a method of treating, preventing, delaying onset of, and/or decreasing the severity of at least one symptom of Huntington's Disease.

In some embodiments, the present disclosure pertains to: A method of manufacture of a medicament comprising a HTT oligonucleotide.

In some embodiments, a HTT oligonucleotide is any individual HTT oligonucleotide or genus of HTT oligonucleotides described herein.

### Base Sequences

In some embodiments, an oligonucleotide, e.g., an HTT oligonucleotide, comprises a base sequence described herein or a portion (e.g., a span of 5-50, 5-40, 5-30, 5-20, or 5, 6, 7, 8, 9, 10, 11, 12, 13, 14, 15, 16, 17, 18, 19, 20, or at least 10, at least 15, contiguous nucleobases) thereof with 0-5 (e.g., 0, 1, 2, 3, 4 or 5) mismatches. In some embodiments, an oligonucleotide, e.g., an HTT oligonucleotide, comprises a base sequence described herein, or a portion thereof, wherein a portion is a span of at least 10 contiguous nucleobases, or a span of at least 15 contiguous nucleobases with 1-5 mismatches. In some embodiments, provided oligonucleotides comprise a base sequence described herein, or a portion thereof, wherein a portion is a span of at least 10 contiguous nucleobases, or a span of at least 10 contiguous nucleobases with 1-5 mismatches. In some embodiments, base sequences of oligonucleotides comprise or consists of 10-50 (e.g., about or at least 10, 11, 12, 13, 14, 15, 16, 17, 18, 19, 20, 21, 22, 23, 24, 25, 30, 35, 40, 45; in some embodiments, at least 15; in some embodiments, at least 16; in some embodiments, at least 17; in some embodiments, at least 18; in some embodiments, at least 19; in some embodiments, at least 20; in some embodiments, at least 21; in some embodiments, at least 22; in some embodiments, at least 23; in some embodiments, at least 24; in some embodiments, at least 25) contiguous bases of a base sequence that is identical to or complementary to a base sequence of an HTT gene or a transcript (e.g., mRNA) thereof.

Base sequences of provided oligonucleotides, as appreciated by those skilled in the art, typically have sufficient length and complementarity to their targets, e.g., RNA transcripts (e.g., pre-mRNA, mature mRNA, etc.) to mediate target-specific knockdown. In some embodiments, the base sequence of an HTT oligonucleotide has a sufficient length and identity to an HTT transcript target to mediate target-specific knockdown. In some embodiments, the HTT oligonucleotide is complementary to a portion of an HTT transcript (a HTT transcript target sequence). In some embodiments, the base sequence of an HTT oligonucleotide has 90% or more identity with the base sequence of an oligonucleotide disclosed in a Table. In some embodiments, the base sequence of an HTT oligonucleotide has 95% or more identity with the base sequence of an oligonucleotide disclosed in a Table. In some embodiments, the base sequence of an HTT oligonucleotide comprises a continuous span of 15 or more bases of an oligonucleotide disclosed in a Table, except that one or more bases within the span are abasic (e.g., a nucleobase is absent from a nucleotide). In some embodiments, the base sequence of an HTT oligonucleotide comprises a continuous span of 19 or more bases of an HTT oligonucleotide disclosed herein, except that one or more bases within the span are abasic (e.g., a nucleobase is absent from a nucleotide). In some embodiments, the base sequence of an HTT oligonucleotide comprises a continuous span of 19 or more bases of an oligonucleotide disclosed herein, except for a difference in the 1 or 2 bases at the 5′ end and/or 3′ end of the base sequences.

In some embodiments, a base sequence of an oligonucleotide is, comprises, or comprises 10-20, e.g., 10, 11, 12, 13, 14, 15, 16, 17, 18, 19 or 20 contiguous bases of TCTCCATTCT ATCTTATGTT, wherein each T may be independently replaced with U.

In some embodiments, a base sequence of an oligonucleotide is, comprises, or comprises 10-20, e.g., 10, 11, 12, 13, 14, 15, 16, 17, 18, 19 or 20 contiguous bases of GTTGATCTGTAGTAGCAGCT or GTTGATCTGTAGCAGCAGCT, wherein each T may be independently replaced with U.

In some embodiments, a base sequence of an oligonucleotide is, comprises, or comprises 10-20, e.g., 10, 11, 12, 13, 14, 15, 16, 17, 18, 19 or 20 contiguous bases of GTGCACACAG TAGATGAGGG, wherein each T may be independently replaced with U.

In some embodiments, a base sequence of an oligonucleotide is, comprises, or comprises 10-20, e.g., 10, 11, 12, 13, 14, 15, 16, 17, 18, 19 or 20 contiguous bases of GTGCAACACA GTAGATGAGGG, wherein each T may be independently replaced with U.

In some embodiments, a base sequence of an oligonucleotide is, comprises, or comprises 10-20, e.g., 10, 11, 12, 13, 14, 15, 16, 17, 18, 19 or 20 contiguous bases of GGCACAAGGG CACAGACTTC, wherein each T may be independently replaced with U.

In some embodiments, a base sequence of an oligonucleotide is, comprises, or comprises 10-20, e.g., 10, 11, 12, 13, 14, 15, 16, 17, 18, 19 or 20 contiguous bases of GGCACAAAGG GCACAGACTTC, wherein each T may be independently replaced with U.

In some embodiments, a base sequence of an oligonucleotide is, comprises, or comprises 10-20, e.g., 10, 11, 12, 13, 14, 15, 16, 17, 18, 19 or 20 contiguous bases of CAAGGGCACA GACTTC, wherein each T may be independently replaced with U.

In some embodiments, a base sequence of an oligonucleotide is, comprises, or comprises 10-20, e.g., 10, 11, 12, 13, 14, 15, 16, 17, 18, 19 or 20 contiguous bases of AAGGGCACAG ACTTC, wherein each T may be independently replaced with U.

In some embodiments, the base sequence of an HTT oligonucleotide is complementary to that of an HTT transcript or a portion thereof.

In some embodiments, an HTT target gene is an allele of the HTT gene. In some embodiments, an HTT oligonucleotide is allele-specific and is designed to target a specific allele of HTT (e.g., an allele associated with an HTT-associated condition, disorder or disease). In some embodiments, the base sequence of an oligonucleotide fully complement the sequence of an HTT transcript (or a portion thereof) from an allele associated with a condition, disorder or disease and is not fully complement the sequence of an HTT transcript (or a portion thereof) less or not associated with a condition, disorder or disease. In some embodiments, a disorder-associated allele of HTT comprises a SNP, mutation or other sequence variation and the HTT oligonucleotide is designed to complement this sequence. In some embodiments, base sequence of an oligonucleotide complement one allele of a SNP and not the others. In some embodiments, base sequence of an oligonucleotide complement one allele of a SNP, which allele is on the same DNA strand of expanded CAG repeats. In some embodiments, the base sequence of an oligonucleotide fully complement the sequence of an HTT transcript (or a portion thereof) from an allele comprising expanded CAG repeats and is not fully complement the sequence of an HTT transcript (or a portion thereof) from an allele comprising normal CAG repeats. In some embodiments, an HTT oligonucleotide is pan-specific and designed to target all alleles of HTT (e.g., all or most known alleles of HTT comprise the same sequence, or a sequence complementary thereto, within the span of bases recognized by the HTT oligonucleotide). In some embodiments, an oligonucleotide reduces expressions, levels and/or activities of both wild-type HTT and mutant HTT, and/or transcripts and/or products thereof.

In some embodiments, an HTT oligonucleotide comprises a base sequence or portion thereof described in the Tables, a sugar, nucleobase, and/or internucleotidic linkage modification described herein, and/or an additional chemical moiety (in addition to an oligonucleotide chain, e.g., a target moiety, a lipid moiety, a carbohydrate moiety, etc.) described herein.

In some embodiments, the terms “complementary,” “fully complementary” and “substantially complementary” may be used with respect to the base matching between an oligonucleotide (e.g., an HTT oligonucleotide) and a target sequence (e.g., an HTT target sequence), as will be understood by those skilled in the art from the context of their use. As a non-limiting example, if a target sequence has, for example, a base sequence of 5′-GCAUAGCGAGCGAGGGAAAAC-3′, an oligonucleotide with a base sequence of 5′GUUUUCCCUCGCUCGCUAUGC-3′ is complementary (fully complementary) to such a target sequence. It is noted that substitution of T for U, or vice versa, generally does not alter the amount of complementarity. As used herein, an oligonucleotide that is “substantially complementary” to a target sequence is largely or mostly complementary but not 100% complementary. In some embodiments, a sequence (e.g., an HTT oligonucleotide) which is substantially complementary has 1, 2, 3, 4 or 5 mismatches when aligned to its target sequence. In some embodiments, an HTT oligonucleotide has a base sequence which is substantially complementary to an HTT target sequence. In some embodiments, an HTT oligonucleotide has a base sequence which is substantially complementary to the complement of the sequence of an HTT oligonucleotide disclosed herein. As appreciated by those skilled in the art, in some embodiments, sequences of oligonucleotides need not be 100% complementary to their targets for the oligonucleotides to perform their functions (e.g., knockdown of target HTT nucleic acids. In some embodiments, homology, sequence identity or complementarity is 60%-100%, e.g., about or at least 60%, 70%, 75%, 80%, 85%, 90%, 91%, 92%, 93%, 94%, 95%, 96%, 97%, 98%, or 99%, or 100%. In some embodiments, a provided oligonucleotide has 75%-100% (e.g., about or at least 80%, 85%, 90%, 91%, 92%, 93%, 94%, 95%, 96%, 97%, 98%, or 99%, or 100%) sequence complementarity to a target region (e.g., a target sequence) within its target HTT nucleic acid. In some embodiments, the percentage is about 80% or more. In some embodiments, the percentage is about 85% or more. In some embodiments, the percentage is about 90% or more. In some embodiments, the percentage is about 95% or more. For example, a provided oligonucleotide which is 20 nucleobases long will have 90 percent complementarity if 18 of its 20 nucleobases are complementary. Typically when determining complementarity, A and T (or U) are complementary nucleobases and C and G are complementary nucleobases.

In some embodiments, the present disclosure provides an HTT oligonucleotide comprising a sequence found in an oligonucleotide described in a Table. In some embodiments, the present disclosure provides an HTT oligonucleotide comprising a sequence found in an oligonucleotide described in a Table, wherein one or more U is independently and optionally replaced with T or vice versa. In some embodiments, an HTT oligonucleotide can comprise at least one T and/or at least one U. In some embodiments, the present disclosure provides an HTT oligonucleotide comprising a sequence found in an oligonucleotide described in a Table, wherein the said sequence has over 50% identity with the sequence of the oligonucleotide described in the Table. In some embodiments, the present disclosure provides an HTT oligonucleotide comprising the sequence of an oligonucleotide disclosed in a Table. In some embodiments, the present disclosure provides an HTT oligonucleotide whose base sequence is the sequence of an oligonucleotide disclosed in a Table. In some embodiments, the present disclosure provides an HTT oligonucleotide comprising a sequence found in an oligonucleotide in a Table, wherein the oligonucleotides have a pattern of backbone linkages, pattern of backbone chiral centers, and/or pattern of backbone phosphorus modifications of the same oligonucleotide or another oligonucleotide in a Table herein.

Among other things, the present disclosure presents, in Table 1 and elsewhere, various oligonucleotides, each of which has a defined base sequence. In some embodiments, the present disclosure, the present disclosure provides an oligonucleotide whose base sequence which is, comprises, or comprises a portion of the base sequence of an oligonucleotide disclosed herein, e.g., in a Table, e.g., Table 1 herein. In some embodiments, the disclosure provides an oligonucleotide having a base sequence which is, comprises, or comprises a portion of the base sequence of an oligonucleotide disclosed herein, e.g., in a Table, wherein the oligonucleotide further comprises a chemical modification, stereochemistry, format, an additional chemical moiety described herein (e.g., a targeting moiety, lipid moiety, carbohydrate moiety, etc.), and/or another structural feature.

In some embodiments, a “portion” (e.g., of a base sequence or a pattern of modifications) is at least 5, 6, 7, 8, 9, 10, 11, 12, 13, 14, 15, 16, 17, 18, 19, or 20 monomeric units long (e.g., for a base sequence, at least 5, 6, 7, 8, 9, 10, 11, 12, 13, 14, 15, 16, 17, 18, 19, or 20 bases long). In some embodiments, a “portion” of a base sequence is at least 5 bases long. In some embodiments, a “portion” of a base sequence is at least 10 bases long. In some embodiments, a “portion” of a base sequence is at least 15 bases long. In some embodiments, a “portion” of a base sequence is at least 20 bases long. In some embodiments, a portion of a base sequence is 10, 11, 12, 13, 14, 15, 16, 17, 18, 19 or more contiguous (consecutive) bases. In some embodiments, a portion of a base sequence is 15 or more contiguous (consecutive) bases.

In some embodiments, the present disclosure provides an oligonucleotide (e.g., an HTT oligonucleotide) whose base sequence is a base sequence of an oligonucleotide in a Table or a portion thereof. In some embodiments, the present disclosure provides an HTT oligonucleotide of a sequence of an oligonucleotide in a Table, wherein the oligonucleotide is capable of directing a decrease in the expression, level and/or activity of an HTT gene or a gene product thereof. As appreciated by those skilled in the art, in provided base sequence, each U may be optionally and independently replaced by T or vice versa, and a sequence can comprise a mixture of U and T. In some embodiments, C may be optionally and independently replaced with 5mC.

In some embodiments, a portion is a span of at least 15, 16, 17, 18, 19, 20, 21, 22, 23, 24, or 25 total nucleotides. In some embodiments, a portion is a span of at least 15, 16, 17, 18, 19, 20, 21, 22, 23, 24, or 25 total nucleotides with 0-3 mismatches. In some embodiments, a portion is a span of at least 15, 16, 17, 18, 19, 20, 21, 22, 23, 24, or 25 total nucleotides with 0-3 mismatches, wherein a span with 0 mismatches is complementary and a span with 1 or more mismatches is a non-limiting example of substantial complementarity. In some embodiments, a base comprises a portion characteristic of a nucleic acid (e.g., a gene) in that the portion is identical or complementary to a portion of the nucleic acid or a transcript thereof, and is not identical or complementary to a portion of any other nucleic acid (e.g., a gene) or a transcript thereof in the same genome. In some embodiments, a portion is characteristic of human HTT. In some embodiments, a portion is characteristic of human mHTT.

In some embodiments, an HTT oligonucleotide has a length of no more than about 49, 45, 40, 30, 35, 25, or 23 total nucleotides as described herein. In some embodiments, wherein the sequence recited herein starts with a U or T at the 5′-end, the U can be deleted and/or replaced by another base. In some embodiments, an oligonucleotide has a base sequence which is or comprises or comprises a portion of the base sequence of an oligonucleotide in a Table, which has a format or a portion of a format disclosed herein.

In some embodiments, oligonucleotides, e.g., HTT oligonucleotides are stereorandom. In some embodiments, oligonucleotides, e.g., HTT oligonucleotides, are chirally controlled. In some embodiments, an oligonucleotide, e.g., an HTT oligonucleotide, is chirally pure (or “stereopure”, “stereochemically pure”), wherein the oligonucleotide exists as a single stereoisomeric form (in many cases a single diastereoisomeric (or “diastereomeric”) form as multiple chiral centers may exist in an oligonucleotide, e.g., at linkage phosphorus, sugar carbon, etc.). As appreciated by those skilled in the art, a chirally pure oligonucleotide is separated from its other stereoisomeric forms (to the extent that some impurities may exist as chemical and biological processes, selectivities and/or purifications etc. rarely, if ever, go to absolute completeness). In a chirally pure oligonucleotide, each chiral center is independently defined with respect to its configuration (stereodefined or chirally controlled, e.g., for chiral linkage phosphorus in chiral internucleotidic linkages, Rp or Sp (such internucleotidic linkages are stereodefined internucleotidic linkages or chirally controlled internucleotidic linkages)). In contrast to chirally controlled and chirally pure oligonucleotides which comprise stereodefined linkage phosphorus, racemic (or “stereorandom”, “non-chirally controlled”) oligonucleotides comprising chiral linkage phosphorus, e.g., from traditional phosphoramidite oligonucleotide synthesis without stereochemical control during coupling steps in combination with traditional sulfurization (creating stereorandom phosphorothioate internucleotidic linkages), refer to a random mixture of various stereoisomers (typically diastereoisomers (or “diastereomers”) as there are multiple chiral centers in an oligonucleotide). For example, for A*A*A wherein * is a phosphorothioate internucleotidic linkage (which comprises a chiral linkage phosphorus), a racemic oligonucleotide preparation includes four diastereomers [2²=4, considering the two chiral linkage phosphorus, each of which can exist in either of two configurations (Sp or Rp)]: A *S A *S A, A *S A *R A, A *R A *S A, and A *R A *R A, wherein *S represents a Sp phosphorothioate internucleotidic linkage and *R represents a Rp phosphorothioate internucleotidic linkage. For a chirally pure oligonucleotide, e.g., A *S A *S A, it exists in a single stereoisomeric form and it is separated from the other stereoisomers (e.g., the diastereomers A *S A *R A, A *R A *S A, and A *R A *R A). In some embodiments, a Rp phosphorothioate is rendered as *S or * S. In some embodiments, a Rp phosphorothioate is rendered as *R or * R.

In some embodiments, oligonucleotides, e.g., HTT oligonucleotides, comprise 1, 2, 3, 4, 5, 6, 7, 8, 9, 10 or more stereorandom internucleotidic linkages (mixture of Rp and Sp linkage phosphorus at the internucleotidic linkage, e.g., from traditional non-chirally controlled oligonucleotide synthesis). In some embodiments, oligonucleotides, e.g., HTT oligonucleotides, comprise one or more (e.g., 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25 or more) chirally controlled internucleotidic linkages (Rp or Sp linkage phosphorus at the internucleotidic linkage, e.g., from chirally controlled oligonucleotide synthesis). In some embodiments, an internucleotidic linkage is a phosphorothioate internucleotidic linkage. In some embodiments, an internucleotidic linkage is a stereorandom phosphorothioate internucleotidic linkage. In some embodiments, an internucleotidic linkage is a chirally controlled phosphorothioate internucleotidic linkage.

Among other things, the present disclosure provides technologies for preparing chirally controlled (in some embodiments, stereochemically pure) oligonucleotides. In some embodiments, oligonucleotides are stereochemically pure. In some embodiments, oligonucleotides of the present disclosure are about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 15%, 20%, 25%, 30%, 35%, 40%, 45%, 50%, 55%, 60%, 65%, 70%, 75%, 80%, 85%, 90%, 95%, or 100%, or at least about 5%, 10%, 15%, 20%, 25%, 30%, 35%, 40%, 45%, 50%, 55%, 60%, 65%, 70%, 75%, 80%, 85%, 90%, 95%, or 99%, pure. In some embodiments, internucleotidic linkages of oligonucleotides comprise or consist of one or more (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more) chiral internucleotidic linkages, each of which independently has a diastereopurity of at least 80%, 85%, 90%, 91%, 92%, 93%, 94%, 95%, 96%, 97%, 98%, 99% or 99.5%, typically at least 90%, 91%, 92%, 93%, 94%, 95%, 96%, 97%, 98%, 99% or 99.5%. In some embodiments, oligonucleotides of the present disclosure, e.g., HTT oligonucleotides, have a diastereopurity of (DS)^{CIL}, wherein DS is a diastereopurity as described in the present disclosure (e.g., 90%, 91%, 92%, 93%, 94%, 95%, 96%, 97%, 98%, 99% or 99.5% or more) and CIL is the number of chirally controlled internucleotidic linkages (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In some embodiments, DS is 95%-100%. In some embodiments, each internucleotidic linkage is independently chirally controlled, and CIL is the number of chirally controlled internucleotidic linkages.

Various HTT oligonucleotides are described and/or referenced herein.

The base sequences and structures of various HTT oligonucleotides, including but not limited to: ONT-450, ONT-451, ONT-452, ONT-453, ONT-454, WV-902, WV-903, WV-904, WV-905, WV-906, WV-907, WV-908, WV-909, WV-910, WV-911, WV-912, WV-913, WV-914, WV-915, WV-916, WV-917, WV-918, WV-919, WV-920, WV-921, WV-922, WV-923, WV-924, WV-925, WV-926, WV-927, WV-928, WV-929, WV-930, WV-931, WV-932, WV-933, WV-934, WV-935, WV-936, WV-937, WV-938, WV-939, WV-940, WV-941, WV-944, WV-945, WV-948, WV-949, WV-950, WV-951, WV-952, WV-953, WV-954, WV-955, WV-956, WV-957, WV-958, WV-959, WV-960, WV-961, WV-962, WV-963, WV-964, WV-965, WV-973, WV-974, WV-975, WV-982, WV-983, WV-984, WV-985, WV-986, WV-987, WV-1001, WV-1002, WV-1003, WV-1004, WV-1005, WV-1006, WV-1007, WV-1008, WV-1009, WV-1010, WV-1011, WV-1012, WV-1013, WV-1014, WV-1015, WV-1016, WV-1017, WV-1018, WV-1019, WV-1020, WV-1021, WV-1022, WV-1023, WV-1024, WV-1025, WV-1026, WV-1027, WV-1028, WV-1029, WV-1030, WV-1031, WV-1032, WV-1033, WV-1034, WV-1035, WV-1036, WV-1037, WV-1038, WV-1039, WV-1040, WV-1041, WV-1042, WV-1043, WV-1044, WV-1045, WV-1046, WV-1047, WV-1048, WV-1049, WV-1050, WV-1051, WV-1052, WV-1053, WV-1054, WV-1055, WV-1056, WV-1057, WV-1058, WV-1059, WV-1060, WV-1061, WV-1062, WV-1063, WV-1064, WV-1065, WV-1066, WV-1067, WV-1068, WV-1069, WV-1070, WV-1071, WV-1072, WV-1073, WV-1074, WV-1075, WV-1076, WV-1077, WV-1078, WV-1079, WV-1080, WV-1081, WV-1082, WV-1083, WV-1084, WV-1085, WV-1086, WV-1087, WV-1088, WV-1089, WV-1090, WV-1091, WV-1092, WV-1234, WV-1235, WV-1497, WV-1508, WV-1509, WV-1510, WV-1511, WV-1654, WV-1655, WV-1788, WV-1789, WV-1790, WV-1799, WV-2022, WV-2023, WV-2024, WV-2025, WV-2026, WV-2027, WV-2028, WV-2029, WV-2030, WV-2031, WV-2032, WV-2033, WV-2034, WV-2035, WV-2036, WV-2037, WV-2038, WV-2039, WV-2040, WV-2041, WV-2042, WV-2043, WV-2044, WV-2045, WV-2046, WV-2047, WV-2048, WV-2049, WV-2050, WV-2051, WV-2052, WV-2053, WV-2054, WV-2055, WV-2056, WV-2057, WV-2058, WV-2059, WV-2060, WV-2061, WV-2062, WV-2063, WV-2064, WV-2065, WV-2066, WV-2067, WV-2068, WV-2069, WV-2070, WV-2071, WV-2072, WV-2073, WV-2074, WV-2075, WV-2076, WV-2077, WV-2078, WV-2079, WV-2080, WV-2081, WV-2082, WV-2083, WV-2084, WV-2085, WV-2086, WV-2087, WV-2088, WV-2089, WV-2090, WV-2163, WV-2164, WV-2269, WV-2270, WV-2271, WV-2272, WV-2374, WV-2375, WV-2376, WV-2377, WV-2378, WV-2379, WV-2380, WV-2416, WV-2417, WV-2418, WV-2419, WV-2431, WV-2589, WV-2590, WV-2591, WV-2592, WV-2593, WV-2594, WV-2595, WV-2596, WV-2597, WV-2598, WV-2599, WV-2600, WV-2601, WV-2602, WV-2603, WV-2604, WV-2605, WV-2606, WV-2607, WV-2608, WV-2609, WV-2610, WV-2611, WV-2612, WV-2613, WV-2614, WV-2615, WV-2616, WV-2617, WV-2618, WV-2619, WV-2620, WV-2623, WV-2638, WV-2639, WV-2640, WV-2641, WV-2642, WV-2643, WV-2659, WV-2671, WV-2672, WV-2673, WV-2674, WV-2675, WV-2676, WV-2682, WV-2683, WV-2684, WV-2685, WV-2686, WV-2687, WV-2688, WV-2689, WV-2690, WV-2691, WV-2692, and WV-2732 are described in WO 2017/015555 and WO 2017/192664, of which the disclosures related to these oligonucleotides are incorporated by reference. Additional HTT oligonucleotides are described herein.

As examples, certain HTT oligonucleotides comprising certain example base sequences, nucleobase modifications and patterns thereof, sugar modifications and patterns thereof, internucleotidic linkages and patterns thereof, linkage phosphorus stereochemistry and patterns thereof, linkers, and/or additional chemical moieties are presented in Table 1, below. Among other things, these oligonucleotides may be utilized to target an HTT transcript, e.g., to reduce the level of an HTT transcript and/or a product thereof.

TABLE 1
Example HTT Oligonucleotides.

| Oligo- | | | Stereochemistry/ |
|---|---|---|---|
| nucleotide | Description | Base Sequence | Linkage |
| WV- | mGmAmAmGmUmCmUmGmUmGmCmCmCmUm | UGAAGUCUGUGCCCUUGUGCC | OOOOO OOOOO |
| 3119 | mGmUmGmCmC | | OOOOO OOOO |
| WV- | mG * SmUmGmCmA * SC * SA * SC * SA * SG * ST * SA * | GUGCACACAGTAGATGAGGG | SOOOS SSSSS SSSSS |
| 3857 | SG * SA * ST * SmGmAmGmG * SmG | | OOOS |
| WV- | mC * SA * SA * SG * SG * SG * SC * SA * SC * RA * SG * | CAAGGGCACAGACUUC | SSSSS SSSRS SOOOS |
| 4241 | SmAmCmUmU * SmC | | |
| WV- | mG * SmGmCmAmC * SA * SA * SG * SG * SG * SC * SA | GGCACAAGGGCA | SOOOS SSSSS S |
| 4242 | | | |
| WV- | mG * SmUmGmCmA * SC * SA * SC * SA * SG * ST * SA * | GUGCACACAGTAGATG | SOOOS SSSSS SSRSS |
| 4243 | SG * RA * ST * SmG | | |
| WV- | mG * SmUmGmCmA * SC * SA * SC * SA * SG * ST * SA | GUGCACACAGTA | SOOOS SSSSS S |
| 4244 | | | |
| WV- | mG * SmGmCmA * SmC * SA * SA * SG * SG * SG * SC * | GGCACAAGGGCA CAGACUUC | SOOSS SSSSS SSRSS |
| 4278 | SA * SC * RA * SG * SmAmCmUmU * SmC | | OOOS |
| WV- | mG * SmUmGmCmA * SC * SA * SC * SA * SG * ST * SA * | GUGCACACAGTAGATGAGGG | SOOOS SSSSS SSRSS |
| 4279 | SG * RA * ST * SmG * SmAmGmG * SmG | | SOOS |
| WV- | T * fA * mCfA * mGfA * mCfU * mUfC * mCfA * mAfA * | TACAGACUUCCAAAGGCUCTU | XXOXO XOXOX |
| 5135 | mG * fG * mC * fU * mC * T * mU | | OXOXX XXXXX |
| WV- | T * fC * mAfC * mAfG * mAfC * mUfU * mCfC * mAfA * | TCACAGACUUCCAAAGGCUTU | XXOXO XOXOX |
| 5136 | mA * fG * mG * fC * mU * T * mU | | OXOXX XXXXX |
| WV- | T * fG * mCfA * mCfA * mGfA * mCfU * mUfC * mCfA * | TGCACAGACUUCCAAAGGCTU | XXOXO XOXOX |
| 5137 | mA * fA * mG * fG * mC * T * mU | | OXOXX XXXXX |
| WV- | T * fG * mGfC * mAfC * mAfG * mAfC * mUfU * mCfC * | TGGCACAGACUUCCAAAGGTU | XXOXO XOXOX |
| 5138 | mA * fA * mA * fG * mG * T * mU | | OXOXX XXXXX |
| WV- | T * fG * mGfG * mCfA * mCfA * mGfA * mCfU * mUfC * | TGGGCACAGACUUCCAAAGTU | XXOXO XOXOX |
| 5139 | mC * fA * mA * fA * mG * T * mU | | OXOXX XXXXX |
| WV- | T * fA * mGfG * mGfC * mAfC * mAfG * mAfC * mUfU * | TAGGGCACAGACUUCCAAATU | XXOXO XOXOX |
| 5140 | mC * fC * mA * fA * mA * T * mU | | OXOXX XXXXX |
| WV- | T * fA * mAfG * mGfG * mCfA * mCfA * mGfA * mCfU * | TAAGGGCACAGACUUCCAATU | XXOXO XOXOX |
| 5141 | mU * fC * mC * fA * mA * T * mU | | OXOXX XXXXX |
| WV- | T * fC * mAfA * mGfG * mGfC * mAfC * mAfG * mAfC * | TCAAGGGCACAGACUUCCATU | XXOXO XOXOX |
| 5142 | mU * fU * mC * fC * mA * T * mU | | OXOXX XXXXX |
| WV- | T * fA * mCfA * mAfG * mGfG * mCfA * mCfA * mGfA * | TACAAGGGCACAGACUUCCTU | XXOXO XOXOX |
| 5143 | mC * fU * mU * fC * mC * T * mU | | OXOXX XXXXX |
| WV- | T * fC * mAfC * mAfA * mGfG * mGfC * mAfC * mAfG * | TCACAAGGGCACAGACUUCTU | XXOXO XOXOX |
| 5144 | mA * fC * mU * fU * mC * T * mU | | OXOXX XXXXX |
| WV- | T * fG * mCfA * mCfA * mAfG * mGfG * mCfA * mCfA * | TGCACAAGGGCA CAGACUUTU | XXOXO XOXOX |
| 5145 | mG * fA * mC * fU * mU * T * mU | | OXOXX XXXXX |
| WV- | T * fG * mGfC * mAfC * mAfA * mGfG * mGfC * mAfC * | TGGCACAAGGGCA CAGACUTU | XXOXO XOXOX |
| 5146 | mA * fG * mA * fC * mU * T * mU | | OXOXX XXXXX |
| WV- | T * fG * mGfG * mCfA * mCfA * mAfG * mGfG * mCfA * | TGGGCACAAGGGCA CAGACTU | XXOXO XOXOX |
| 5147 | mC * fA * mG * fA * mC * T * mU | | OXOXX XXXXX |
| WV- | T * fA * mGfG * mGfC * mAfC * mAfA * mGfG * mGfC * | TAGGGCACAAGGGCA CAGATU | XXOXO XOXOX |
| 5148 | mA * fC * mA * fG * mA * T * mU | | OXOXX XXXXX |
| WV- | T * fC * mAfG * mGfG * mCfA * mCfA * mAfG * mGfG * | TCAGGGCACAAGGGCA CAGTU | XXOXO XOXOX |
| 5149 | mC * fA * mC * fA * mG * T * mU | | OXOXX XXXXX |
| WV- | T * fG * mCfA * mGfG * mGfC * mAfC * mAfA * mGfG * | TGCAGGGCACAAGGGCA CATU | XXOXO XOXOX |
| 5150 | mG * fC * mA * fC * mA * T * mU | | OXOXX XXXXX |
| WV- | T * fG * mGfC * mAfG * mGfG * mCfA * mCfA * mAfG * | TGGCAGGGCACAAGGGCA CTU | XXOXO XOXOX |
| 5151 | mG * fG * mC * fA * mC * T * mU | | OXOXX XXXXX |
| WV- | T * fA * mGfG * mCfA * mGfG * mGfC * mAfC * mAfA * | TAGGCAGGGCACAAGGGCA TU | XXOXO XOXOX |
| 5152 | mG * fG * mG * fC * mA * T * mU | | OXOXX XXXXX |
| WV- | T * fA * mCfA * mGfA * mCfU * mUfC * mCfA * mAfA * | TACAGACUUCCAAAGGCUCTU | XXOXO XOXOX |
| 5153 | mGfGmCfUmC * T * mU | | OXOXO OOOXX |
| WV- | T * fC * mAfC * mAfG * mAfC * mUfU * mCfC * mAfA * | TCACAGACUUCCAAAGGCUTU | XXOXO XOXOX |
| 5154 | mAfGmGfCmU * T * mU | | OXOXO OOOXX |
| WV- | T * fG * mCfA * mCfA * mGfA * mCfU * mUfC * mCfA * | TGCACAGACUUCCAAAGGCTU | XXOXO XOXOX |
| 5155 | mAfAmGfGmC * T * mU | | OXOXO OOOXX |
| WV- | T * fG * mGfC * mAfC * mAfG * mAfC * mUfU * mCfC * | TGGCACAGACUUCCAAAGGTU | XXOXO XOXOX |
| 5156 | mAfAmAfGmG * T * mU | | OXOXO OOOXX |
| WV- | T * fG * mGfG * mCfA * mCfA * mGfA * mCfU * mUfC * | TGGGCACAGACUUCCAAAGTU | XXOXO XOXOX |
| 5157 | mCfAmAfAmG * T * mU | | OXOXO OOOXX |
| WV- | T * fA * mGfG * mGfC * mAfC * mAfG * mAfC * mUfU * | TAGGGCACAGACUUCCAAATU | XXOXO XOXOX |
| 5158 | mCfCmAfAmA * T * mU | | OXOXO OOOXX |
| WV- | T * fA * mAfG * mGfG * mCfA * mCfA * mGfA * mCfU * | TAAGGGCACAGACUUCCAATU | XXOXO XOXOX |
| 5159 | mUfCmCfAmA * T * mU | | OXOXO OOOXX |
| WV- | T * fC * mAfA * mGfG * mGfC * mAfC * mAfG * mAfC * | TCAAGGGCACAGACUUCCATU | XXOXO XOXOX |
| 5160 | mUfUmCfCmA * T * mU | | OXOXO OOOXX |
| WV- | T * fA * mCfA * mAfG * mGfG * mCfA * mCfA * mGfA * | TACAAGGGCACAGACUUCCTU | XXOXO XOXOX |
| 5161 | mCfUmUfCmC * T * mU | | OXOXO OOOXX |
| WV- | T * fC * mAfC * mAfA * mGfG * mGfC * mAfC * mAfG * | TCACAAGGGCACAGACUUCTU | XXOXO XOXOX |
| 5162 | mAfCmUfUmC * T * mU | | OXOXO OOOXX |
| WV- | T * fG * mCfA * mCfA * mAfG * mGfG * mCfA * mCfA * | TGCACAAGGGCA CAGACUUTU | XXOXO XOXOX |
| 5163 | mGfAmCfUmU * T * mU | | OXOXO OOOXX |
| WV- | T * fG * mGfC * mAfC * mAfA * mGfG * mGfC * mAfC * | TGGCACAAGGGCA CAGACUTU | XXOXO XOXOX |
| 5164 | mAfGmAfCmU * T * mU | | OXOXO OOOXX |
| WV- | T * fG * mGfG * mCfA * mCfA * mAfG * mGfG * mCfA * | TGGGCACAAGGGCA CAGACTU | XXOXO XOXOX |
| 5165 | mCfAmGfAmC * T * mU | | OXOXO OOOXX |
| WV- | T * fA * mGfG * mGfC * mAfC * mAfA * mGfG * mGfC * | TAGGGCACAAGGGCA CAGATU | XXOXO XOXOX |
| 5166 | mAfCmAfGmA * T * mU | | OXOXO OOOXX |
| WV- | T * fC * mAfG * mGfG * mCfA * mCfA * mAfG * mGfG * | TCAGGGCACAAGGGCA CAGTU | XXOXO XOXOX |
| 5167 | mCfAmCfAmG * T * mU | | OXOXO OOOXX |
| WV- | T * fG * mCfA * mGfG * mGfC * mAfC * mAfA * mGfG * | TGCAGGGCACAAGGGCA CATU | XXOXO XOXOX |
| 5168 | mGfCmAfCmA * T * mU | | OXOXO OOOXX |
| WV- | T * fG * mGfC * mAfG * mGfG * mCfA * mCfA * mAfG * | TGGCAGGGCACAAGGGCA CTU | XXOXO XOXOX |
| 5169 | mGfGmCfAmC * T * mU | | OXOXO OOOXX |
| WV- | T * fA * mGfG * mCfA * mGfG * mGfC * mAfC * mAfA * | TAGGCAGGGCACAAGGGCA TU | XXOXO XOXOX |
| 5170 | mGfGmGfCmA * T * mU | | OXOXO OOOXX |
| WV- | T * fA * mCfA * mGfA * mCfU * mUfC * mCfA * mAfA * | TACAGACUUCCAAAGGCUCCGTU | XXOXO XOXOX |
| 5171 | mG * fG * mC * fU * mC * fC * mG * T * mU | | OXOXX XXXXX XX |
| WV- | T * fC * mAfC * mAfG * mAfC * mUfU * mCfC * mAfA * | TCACAGACUUCCAAAGGCUCCTU | XXOXO XOXOX |
| 5172 | mA * fG * mG * fC * mU * fC * mC * T * mU | | OXOXX XXXXX XX |
| WV- | T * fG * mCfA * mCfA * mGfA * mCfU * mUfC * mCfA * | TGCACAGACUUCCAAAGGCUCTU | XXOXO XOXOX |
| 5173 | mA * fA * mG * fG * mC * fU * mC * T * mU | | OXOXX XXXXX XX |
| WV- | T * fG * mGfC * mAfC * mAfG * mAfC * mUfU * mCfC * | TGGCACAGACUUCCAAAGGCUTU | XXOXO XOXOX |
| 5174 | mA * fA * mA * fG * mG * fC * mU * T * mU | | OXOXX XXXXX XX |
| WV- | T * fG * mGfG * mCfA * mCfA * mGfA * mCfU * mUfC * | TGGGCACAGACUUCCAAAGGCTU | XXOXO XOXOX |
| 5175 | mC * fA * mA * fA * mG * fG * mC * T * mU | | OXOXX XXXXX XX |
| WV- | T * fA * mGfG * mGfC * mAfC * mAfG * mAfC * mUfU * | TAGGGCACAGACUUCCAAAGGTU | XXOXO XOXOX |
| 5176 | mC * fC * mA * fA * mA * fG * mG * T * mU | | OXOXX XXXXX XX |
| WV- | T * fA * mAfG * mGfG * mCfA * mCfA * mGfA * mCfU * | TAAGGGCACAGACUUCCAAAGTU | XXOXO XOXOX |
| 5177 | mU * fC * mC * fA * mA * fA * mG * T * mU | | OXOXX XXXXX XX |
| WV- | T * fC * mAfA * mGfG * mGfC * mAfC * mAfG * mAfC * | TCAAGGGCACAGACUUCCAAATU | XXOXO XOXOX |
| 5178 | mU * fU * mC * fC * mA * fA * mA * T * mU | | OXOXX XXXXX XX |
| WV- | T * fA * mCfA * mAfG * mGfG * mCfA * mCfA * mGfA * | TACAAGGGCACAGACUUCCAATU | XXOXO XOXOX |
| 5179 | mC * fU * mU * fC * mC * fA * mA * T * mU | | OXOXX XXXXX XX |
| WV- | T * fC * mAfC * mAfA * mGfG * mGfC * mAfC * mAfG * | TCACAAGGGCACAGACUUCCATU | XXOXO XOXOX |
| 5180 | mA * fC * mU * fU * mC * fC * mA * T * mU | | OXOXX XXXXX XX |
| WV- | T * fG * mCfA * mCfA * mAfG * mGfG * mCfA * mCfA * | TGCACAAGGGCA CAGACUUCCTU | XXOXO XOXOX |
| 5181 | mG * fA * mC * fU * mU * fC * mC * T * mU | | OXOXX XXXXX XX |
| WV- | T * fG * mGfC * mAfC * mAfA * mGfG * mGfC * mAfC * | TGGCACAAGGGCA | XXOXO XOXOX |
| 5182 | mA * fG * mA * fC * mU * fU * mC * T * mU | CAGACUUCTU | OXOXX XXXXX XX |
| WV- | T * fG * mGfG * mCfA * mCfA * mAfG * mGfG * mCfA * | TGGGCACAAGGGCA | XXOXO XOXOX |
| 5183 | mC * fA * mG * fA * mC * fU * mU * T * mU | CAGACUUTU | OXOXX XXXXX XX |
| WV- | T * fA * mGfG * mGfC * mAfC * mAfA * mGfG * mGfC * | TAGGGCACAAGGGCA | XXOXO XOXOX |
| 5184 | mA * fC * mA * fG * mA * fC * mU * T * mU | CAGACUTU | OXOXX XXXXX XX |
| WV- | T * fC * mAfG * mGfG * mCfA * mCfA * mAfG * mGfG * | TCAGGGCACAAGGGCA | XXOXO XOXOX |
| 5185 | mC * fA * mC * fA * mG * fA * mC * T * mU | CAGACTU | OXOXX XXXXX XX |
| WV- | T * fG * mCfA * mGfG * mGfC * mAfC * mAfA * mGfG * | TGCAGGGCACAAGGGCA | XXOXO XOXOX |
| 5186 | mG * fC * mA * fC * mA * fG * mA * T * mU | CAGATU | OXOXX XXXXX XX |
| WV- | T * fG * mGfC * mAfG * mGfG * mCfA * mCfA * mAfG * | TGGCAGGGCACAAGGGCA | XXOXO XOXOX |
| 5187 | mG * fG * mC * fA * mC * fA * mG * T * mU | CAGTU | OXOXX XXXXX XX |
| WV- | T * fA * mGfG * mCfA * mGfG * mGfC * mAfC * mAfA * | TAGGCAGGGCACAAGGGCA | XXOXO XOXOX |
| 5188 | mG * fG * mG * fC * mA * fC * mA * T * mU | CATU | OXOXX XXXXX XX |
| WV- | T * fG * mAfG * mGfC * mAfG * mGfG * mCfA * mCfA * | TGAGGCAGGGCACAAGGGCA | XXOXO XOXOX |
| 5189 | mA * fG * mG * fG * mC * fA * mC * T * mU | CTU | OXOXX XXXXX XX |
| WV- | T * fG * mGfA * mGfG * mCfA * mGfG * mGfC * mAfC * | TGGAGGCAGGGCACAAGGGCA | XXOXO XOXOX |
| 5190 | mA * fA * mG * fG * mG * fC * mA * T * mU | TU | OXOXX XXXXX XX |
| WV- | T * fA * mCfA * mGfA * mCfU * mUfC * mCfA * mAfA * | TACAGACUUCCAAAGGCUCCGTU | XXOXO XOXOX |
| 5191 | mGfGmCfUmCfCmG * T * mU | | OXOXO OOOOO XX |
| WV- | T * fC * mAfC * mAfG * mAfC * mUfU * mCfC * mAfA * | TCACAGACUUCCAAAGGCUCCTU | XXOXO XOXOX |
| 5192 | mAfGmGfCmUfCmC * T * mU | | OXOXO OOOOO XX |
| WV- | T * fG * mCfA * mCfA * mGfA * mCfU * mUfC * mCfA * | TGCACAGACUUCCAAAGGCUCTU | XXOXO XOXOX |
| 5193 | mAfAmGfGmCfUmC * T * mU | | OXOXO OOOOO XX |
| WV- | T * fG * mGfC * mAfC * mAfG * mAfC * mUfU * mCfC * | TGGCACAGACUUCCAAAGGCUTU | XXOXO XOXOX |
| 5194 | mAfAmAfGmGfCmU * T * mU | | OXOXO OOOOO XX |
| WV- | T * fG * mGfG * mCfA * mCfA * mGfA * mCfU * mUfC * | TGGGCACAGACUUCCAAAGGCTU | XXOXO XOXOX |
| 5195 | mCfAmAfAmGfGmC * T * mU | | OXOXO OOOOO XX |
| WV- | T * fA * mGfG * mGfC * mAfC * mAfG * mAfC * mUfU * | TAGGGCACAGACUUCCAAAGGTU | XXOXO XOXOX |
| 5196 | mCfCmAfAmAfGmG * T * mU | | OXOXO OOOOO XX |
| WV- | T * fA * mAfG * mGfG * mCfA * mCfA * mGfA * mCfU * | TAAGGGCACAGACUUCCAAAGTU | XXOXO XOXOX |
| 5197 | mUfCmCfAmAfAmG * T * mU | | OXOXO OOOOO XX |
| WV- | T * fC * mAfA * mGfG * mGfC * mAfC * mAfG * mAfC * | TCAAGGGCACAGACUUCCAAATU | XXOXO XOXOX |
| 5198 | mUfUmCfCmAfAmA * T * mU | | OXOXO OOOOO XX |
| WV- | T * fA * mCfA * mAfG * mGfG * mCfA * mCfA * mGfA * | TACAAGGGCACAGACUUCCAATU | XXOXO XOXOX |
| 5199 | mCfUmUfCmCfAmA * T * mU | | OXOXO OOOOO XX |
| WV- | T * fC * mAfC * mAfA * mGfG * mGfC * mAfC * mAfG * | TCACAAGGGCACAGACUUCCATU | XXOXO XOXOX |
| 5200 | mAfCmUfUmCfCmA * T * mU | | OXOXO OOOOO XX |
| WV- | T * fG * mCfA * mCfA * mAfG * mGfG * mCfA * mCfA * | TGCACAAGGGCA CAGACUUCCTU | XXOXO XOXOX |
| 5201 | mGfAmCfUmUfCmC * T * mU | | OXOXO OOOOO XX |
| WV- | T * fG * mGfC * mAfC * mAfA * mGfG * mGfC * mAfC * | TGGCACAAGGGCA | XXOXO XOXOX |
| 5202 | mAfGmAfCmUfUmC * T * mU | CAGACUUCTU | OXOXO OOOOO XX |
| WV- | T * fG * mGfG * mCfA * mCfA * mAfG * mGfG * mCfA * | TGGGCACAAGGGCA | XXOXO XOXOX |
| 5203 | mCfAmGfAmCfUmU * T * mU | CAGACUUTU | OXOXO OOOOO XX |
| WV- | T * fA * mGfG * mGfC * mAfC * mAfA * mGfG * mGfC * | TAGGGCACAAGGGCA | XXOXO XOXOX |
| 5204 | mAfCmAfGmAfCmU * T * mU | CAGACUTU | OXOXO OOOOO XX |
| WV- | T * fC * mAfG * mGfG * mCfA * mCfA * mAfG * mGfG * | TCAGGGCACAAGGGCA | XXOXO XOXOX |
| 5205 | mCfAmCfAmGfAmC * T * mU | CAGACTU | OXOXO OOOOO XX |
| WV- | T * fG * mCfA * mGfG * mGfC * mAfC * mAfA * mGfG * | TGCAGGGCACAAGGGCA | XXOXO XOXOX |
| 5206 | mGfCmAfCmAfGmA * T * mU | CAGATU | OXOXO OOOOO XX |
| WV- | T * fG * mGfC * mAfG * mGfG * mCfA * mCfA * mAfG * | TGGCAGGGCACAAGGGCA | XXOXO XOXOX |
| 5207 | mGfGmCfAmCfAmG * T * mU | CAGTU | OXOXO OOOOO XX |
| WV- | T * fA * mGfG * mCfA * mGfG * mGfC * mAfC * mAfA * | TAGGCAGGGCACAAGGGCA | XXOXO XOXOX |
| 5208 | mGfGmGfCmAfCmA * T * mU | CATU | OXOXO OOOOO XX |
| WV- | T * fG * mAfG * mGfC * mAfG * mGfG * mCfA * mCfA * | TGAGGCAGGGCACAAGGGCA | XXOXO XOXOX |
| 5209 | mAfGmGfGmCfAmC * T * mU | CTU | OXOXO OOOOO XX |
| WV- | T * fG * mGfA * mGfG * mCfA * mGfG * mGfC * mAfC * | TGGAGGCAGGGCACAAGGGCA | XXOXO XOXOX |
| 5210 | mAfAmGfGmGfCmA * T * mU | TU | OXOXO OOOOO XX |
| WV- | T * fA * mGfA * mUfG * mAfG * mGfG * mAfG * mCfA * | TAGAUGAGGGAGCAGGCGUTU | XXOXO XOXOX |
| 5211 | mG * fG * mC * fG * mU * T * mU | | OXOXX XXXXX |
| WV- | T * fU * mAfG * mAfU * mGfA * mGfG * mGfA * mGfC * | TUAGAUGAGGGAGCAGGCGTU | XXOXO XOXOX |
| 5212 | mA * fG * mG * fC * mG * T * mU | | OXOXX XXXXX |
| WV- | T * fG * mUfA * mGfA * mUfG * mAfG * mGfG * mAfG * | TGUAGAUGAGGGAGCAGGCTU | XXOXO XOXOX |
| 5213 | mC * fA * mG * fG * mC * T * mU | | OXOXX XXXXX |
| WV- | T * fA * mGfU * mAfG * mAfU * mGfA * mGfG * mGfA * | TAGUAGAUGAGGGAGCAGGTU | XXOXO XOXOX |
| 5214 | mG * fC * mA * fG * mG * T * mU | | OXOXX XXXXX |
| WV- | T * fC * mAfG * mUfA * mGfA * mUfG * mAfG * mGfG * | TCAGUAGAUGAGGGAGCAGTU | XXOXO XOXOX |
| 5215 | mA * fG * mC * fA * mG * T * mU | | OXOXX XXXXX |
| WV- | T * fA * mCfA * mGfU * mAfG * mAfU * mGfA * mGfG * | TACAGUAGAUGAGGGAGCATU | XXOXO XOXOX |
| 5216 | mG * fA * mG * fC * mA * T * mU | | OXOXX XXXXX |
| WV- | T * fC * mAfC * mAfG * mUfA * mGfA * mUfG * mAfG * | TCACAGUAGAUGAGGGAGCTU | XXOXO XOXOX |
| 5217 | mG * fG * mA * fG * mC * T * mU | | OXOXX XXXXX |
| WV- | T * fA * mCfA * mCfA * mGfU * mAfG * mAfU * mGfA * | TACACAGUAGAUGAGGGAGTU | XXOXO XOXOX |
| 5218 | mG * fG * mG * fA * mG * T * mU | | OXOXX XXXXX |
| WV- | T * fC * mAfC * mAfC * mAfG * mUfA * mGfA * mUfG * | TCACACAGUAGAUGAGGGATU | XXOXO XOXOX |
| 5219 | mA * fG * mG * fG * mA * T * mU | | OXOXX XXXXX |
| WV- | T * fG * mCfA * mCfA * mCfA * mGfU * mAfG * mAfU * | TGCACACAGUAGAUGAGGGTU | XXOXO XOXOX |
| 5220 | mG * fA * mG * fG * mG * T * mU | | OXOXX XXXXX |
| WV- | T * fU * mGfC * mAfC * mAfC * mAfG * mUfA * mGfA * | TUGCACACAGUAGAUGAGGTU | XXOXO XOXOX |
| 5221 | mU * fG * mA * fG * mG * T * mU | | OXOXX XXXXX |
| WV- | T * fG * mUfG * mCfA * mCfA * mCfA * mGfU * mAfG * | TGUGCACACAGUAGAUGAGTU | XXOXO XOXOX |
| 5222 | mA * fU * mG * fA * mG * T * mU | | OXOXX XXXXX |
| WV- | T * fA * mGfU * mGfC * mAfC * mAfC * mAfG * mUfA * | TAGUGCACACAGUAGAUGATU | XXOXO XOXOX |
| 5223 | mG * fA * mU * fG * mA * T * mU | | OXOXX XXXXX |
| WV- | T * fA * mAfG * mUfG * mCfA * mCfA * mCfA * mGfU * | TAAGUGCACACAGUAGAUGTU | XXOXO XOXOX |
| 5224 | mA * fG * mA * fU * mG * T * mU | | OXOXX XXXXX |
| WV- | T * fG * mAfA * mGfU * mGfC * mAfC * mAfC * mAfG * | TGAAGUGCACACAGUAGAUTU | XXOXO XOXOX |
| 5225 | mU * fA * mG * fA * mU * T * mU | | OXOXX XXXXX |
| WV- | T * fU * mGfA * mAfG * mUfG * mCfA * mCfA * mCfA * | TUGAAGUGCACACAGUAGATU | XXOXO XOXOX |
| 5226 | mG * fU * mA * fG * mA * T * mU | | OXOXX XXXXX |
| WV- | T * fA * mUfG * mAfA * mGfU * mGfC * mAfC * mAfC * | TAUGAAGUGCACACAGUAGTU | XXOXO XOXOX |
| 5227 | mA * fG * mU * fA * mG * T * mU | | OXOXX XXXXX |
| WV- | T * fG * mAfU * mGfA * mAfG * mUfG * mCfA * mCfA * | TGAUGAAGUGCACACAGUATU | XXOXO XOXOX |
| 5228 | mC * fA * mG * fU * mA * T * mU | | OXOXX XXXXX |
| WV- | T * fA * mGfA * mUfG * mAfG * mGfG * mAfG * mCfA * | TAGAUGAGGGAGCAGGCGUTU | XXOXO XOXOX |
| 5229 | mGfGmCfGmU * T * mU | | OXOXO OOOXX |
| WV- | T * fU * mAfG * mAfU * mGfA * mGfG * mGfA * mGfC * | TUAGAUGAGGGAGCAGGCGTU | XXOXO XOXOX |
| 5230 | mAfGmGfCmG * T * mU | | OXOXO OOOXX |
| WV- | T * fG * mUfA * mGfA * mUfG * mAfG * mGfG * mAfG * | TGUAGAUGAGGGAGCAGGCTU | XXOXO XOXOX |
| 5231 | mCfAmGfGmC * T * mU | | OXOXO OOOXX |
| WV- | T * fA * mGfU * mAfG * mAfU * mGfA * mGfG * mGfA * | TAGUAGAUGAGGGAGCAGGTU | XXOXO XOXOX |
| 5232 | mGfCmAfGmG * T * mU | | OXOXO OOOXX |
| WV- | T * fC * mAfG * mUfA * mGfA * mUfG * mAfG * mGfG * | TCAGUAGAUGAGGGAGCAGTU | XXOXO XOXOX |
| 5233 | mAfGmCfAmG * T * mU | | OXOXO OOOXX |
| WV- | T * fA * mCfA * mGfU * mAfG * mAfU * mGfA * mGfG * | TACAGUAGAUGAGGGAGCATU | XXOXO XOXOX |
| 5234 | mGfAmGfCmA * T * mU | | OXOXO OOOXX |
| WV- | T * fC * mAfC * mAfG * mUfA * mGfA * mUfG * mAfG * | TCACAGUAGAUGAGGGAGCTU | XXOXO XOXOX |
| 5235 | mGfGmAfGmC * T * mU | | OXOXO OOOXX |
| WV- | T * fA * mCfA * mCfA * mGfU * mAfG * mAfU * mGfA * | TACACAGUAGAUGAGGGAGTU | XXOXO XOXOX |
| 5236 | mGfGmGfAmG * T * mU | | OXOXO OOOXX |
| WV- | T * fC * mAfC * mAfC * mAfG * mUfA * mGfA * mUfG * | TCACACAGUAGAUGAGGGATU | XXOXO XOXOX |
| 5237 | mAfGmGfGmA * T * mU | | OXOXO OOOXX |
| WV- | T * fG * mCfA * mCfA * mCfA * mGfU * mAfG * mAfU * | TGCACACAGUAGAUGAGGGTU | XXOXO XOXOX |
| 5238 | mGfAmGfGmG * T * mU | | OXOXO OOOXX |
| WV- | T * fU * mGfC * mAfC * mAfC * mAfG * mUfA * mGfA * | TUGCACACAGUAGAUGAGGTU | XXOXO XOXOX |
| 5239 | mUfGmAfGmG * T * mU | | OXOXO OOOXX |
| WV- | T * fG * mUfG * mCfA * mCfA * mCfA * mGfU * mAfG * | TGUGCACACAGUAGAUGAGTU | XXOXO XOXOX |
| 5240 | mAfUmGfAmG * T * mU | | OXOXO OOOXX |
| WV- | T * fA * mGfU * mGfC * mAfC * mAfC * mAfG * mUfA * | TAGUGCACACAGUAGAUGATU | XXOXO XOXOX |
| 5241 | mGfAmUfGmA * T * mU | | OXOXO OOOXX |
| WV- | T * fA * mAfG * mUfG * mCfA * mCfA * mCfA * mGfU * | TAAGUGCACACAGUAGAUGTU | XXOXO XOXOX |
| 5242 | mAfGmAfUmG * T * mU | | OXOXO OOOXX |
| WV- | T * fG * mAfA * mGfU * mGfC * mAfC * mAfC * mAfG * | TGAAGUGCACACAGUAGAUTU | XXOXO XOXOX |
| 5243 | mUfAmGfAmU * T * mU | | OXOXO OOOXX |
| WV- | T * fU * mGfA * mAfG * mUfG * mCfA * mCfA * mCfA * | TUGAAGUGCACACAGUAGATU | XXOXO XOXOX |
| 5244 | mGfUmAfGmA * T * mU | | OXOXO OOOXX |
| WV- | T * fA * mUfG * mAfA * mGfU * mGfC * mAfC * mAfC * | TAUGAAGUGCACACAGUAGTU | XXOXO XOXOX |
| 5245 | mAfGmUfAmG * T * mU | | OXOXO OOOXX |
| WV- | T * fG * mAfU * mGfA * mAfG * mUfG * mCfA * mCfA * | TGAUGAAGUGCACACAGUATU | XXOXO XOXOX |
| 5246 | mCfAmGfUmA * T * mU | | OXOXO OOOXX |
| WV- | T * fA * mGfA * mUfG * mAfG * mGfG * mAfG * mCfA * | TAGAUGAGGGAGCAGGCGUGGTU | XXOXO XOXOX |
| 5247 | mG * fG * mC * fG * mU * fG * mG * T * mU | | OXOXX XXXXX XX |
| WV- | T * fU * mAfG * mAfU * mGfA * mGfG * mGfA * mGfC * | TUAGAUGAGGGAGCAGGCGUGTU | XXOXO XOXOX |
| 5248 | mA * fG * mG * fC * mG * fU * mG * T * mU | | OXOXX XXXXX XX |
| WV- | T * fG * mUfA * mGfA * mUfG * mAfG * mGfG * mAfG * | TGUAGAUGAGGGAGCAGGCGUTU | XXOXO XOXOX |
| 5249 | mC * fA * mG * fG * mC * fG * mU * T * mU | | OXOXX XXXXX XX |
| WV- | T * fA * mGfU * mAfG * mAfU * mGfA * mGfG * mGfA * | TAGUAGAUGAGGGAGCAGGCGTU | XXOXO XOXOX |
| 5250 | mG * fC * mA * fG * mG * fC * mG * T * mU | | OXOXX XXXXX XX |
| WV- | T * fC * mAfG * mUfA * mGfA * mUfG * mAfG * mGfG * | TCAGUAGAUGAGGGAGCAGGCTU | XXOXO XOXOX |
| 5251 | mA * fG * mC * fA * mG * fG * mC * T * mU | | OXOXX XXXXX XX |
| WV- | T * fA * mCfA * mGfU * mAfG * mAfU * mGfA * mGfG * | TACAGUAGAUGAGGGAGCAGGTU | XXOXO XOXOX |
| 5252 | mG * fA * mG * fC * mA * fG * mG * T * mU | | OXOXX XXXXX XX |
| WV- | T * fC * mAfC * mAfG * mUfA * mGfA * mUfG * mAfG * | TCACAGUAGAUGAGGGAGCAGTU | XXOXO XOXOX |
| 5253 | mG * fG * mA * fG * mC * fA * mG * T * mU | | OXOXX XXXXX XX |
| WV- | T * fA * mCfA * mCfA * mGfU * mAfG * mAfU * mGfA * | TACACAGUAGAUGAGGGAGCATU | XXOXO XOXOX |
| 5254 | mG * fG * mG * fA * mG * fC * mA * T * mU | | OXOXX XXXXX XX |
| WV- | T * fC * mAfC * mAfC * mAfG * mUfA * mGfA * mUfG * | TCACACAGUAGAUGAGGGAGCTU | XXOXO XOXOX |
| 5255 | mA * fG * mG * fG * mA * fG * mC * T * mU | | OXOXX XXXXX XX |
| WV- | T * fG * mCfA * mCfA * mCfA * mGfU * mAfG * mAfU * | TGCACACAGUAGAUGAGGGAGTU | XXOXO XOXOX |
| 5256 | mG * fA * mG * fG * mG * fA * mG * T * mU | | OXOXX XXXXX XX |
| WV- | T * fU * mGfC * mAfC * mAfC * mAfG * mUfA * mGfA * | TUGCACACAGUAGAUGAGGGATU | XXOXO XOXOX |
| 5257 | mU * fG * mA * fG * mG * fG * mA * T * mU | | OXOXX XXXXX XX |
| WV- | T * fG * mUfG * mCfA * mCfA * mCfA * mGfU * mAfG * | TGUGCACACAGUAGAUGAGGGTU | XXOXO XOXOX |
| 5258 | mA * fU * mG * fA * mG * fG * mG * T * mU | | OXOXX XXXXX XX |
| WV- | T * fA * mGfU * mGfC * mAfC * mAfC * mAfG * mUfA * | TAGUGCACACAGUAGAUGAGGTU | XXOXO XOXOX |
| 5259 | mG * fA * mU * fG * mA * fG * mG * T * mU | | OXOXX XXXXX XX |
| WV- | T * fA * mAfG * mUfG * mCfA * mCfA * mCfA * mGfU * | TAAGUGCACACAGUAGAUGAGTU | XXOXO XOXOX |
| 5260 | mA * fG * mA * fU * mG * fA * mG * T * mU | | OXOXX XXXXX XX |
| WV- | T * fG * mAfA * mGfU * mGfC * mAfC * mAfC * mAfG * | TGAAGUGCACACAGUAGAUGATU | XXOXO XOXOX |
| 5261 | mU * fA * mG * fA * mU * fG * mA * T * mU | | OXOXX XXXXX XX |
| WV- | T * fU * mGfA * mAfG * mUfG * mCfA * mCfA * mCfA * | TUGAAGUGCACACAGUAGAUGTU | XXOXO XOXOX |
| 5262 | mG * fU * mA * fG * mA * fU * mG * T * mU | | OXOXX XXXXX XX |
| WV- | T * fA * mUfG * mAfA * mGfU * mGfC * mAfC * mAfC * | TAUGAAGUGCACACAGUAGAUTU | XXOXO XOXOX |
| 5263 | mA * fG * mU * fA * mG * fA * mU * T * mU | | OXOXX XXXXX XX |
| WV- | T * fG * mAfU * mGfA * mAfG * mUfG * mCfA * mCfA * | TGAUGAAGUGCACACAGUAGATU | XXOXO XOXOX |
| 5264 | mC * fA * mG * fU * mA * fG * mA * T * mU | | OXOXX XXXXX XX |
| WV- | T * fG * mGfA * mUfG * mAfA * mGfU * mGfC * mAfC * | TGGAUGAAGUGCACACAGUAGTU | XXOXO XOXOX |
| 5265 | mA * fC * mA * fG * mU * fA * mG * T * mU | | OXOXX XXXXX XX |
| WV- | T * fA * mGfG * mAfU * mGfA * mAfG * mUfG * mCfA * | TAGGAUGAAGUGCACACAGUATU | XXOXO XOXOX |
| 5266 | mC * fA * mC * fA * mG * fU * mA * T * mU | | OXOXX XXXXX XX |
| WV- | T * fA * mGfA * mUfG * mAfG * mGfG * mAfG * mCfA * | TAGAUGAGGGAGCAGGCGUGGTU | XXOXO XOXOX |
| 5267 | mGfGmCfGmUfGmG * T * mU | | OXOXO OOOOO XX |
| WV- | T * fU * mAfG * mAfU * mGfA * mGfG * mGfA * mGfC * | TUAGAUGAGGGAGCAGGCGUGTU | XXOXO XOXOX |
| 5268 | mAfGmGfCmGfUmG * T * mU | | OXOXO OOOOO XX |
| WV- | T * fG * mUfA * mGfA * mUfG * mAfG * mGfG * mAfG * | TGUAGAUGAGGGAGCAGGCGUTU | XXOXO XOXOX |
| 5269 | mCfAmGfGmCfGmU * T * mU | | OXOXO OOOOO XX |
| WV- | T * fA * mGfU * mAfG * mAfU * mGfA * mGfG * mGfA * | TAGUAGAUGAGGGAGCAGGCGTU | XXOXO XOXOX |
| 5270 | mGfCmAfGmGfCmG * T * mU | | OXOXO OOOOO XX |
| WV- | T * fC * mAfG * mUfA * mGfA * mUfG * mAfG * mGfG * | TCAGUAGAUGAGGGAGCAGGCTU | XXOXO XOXOX |
| 5271 | mAfGmCfAmGfGmC * T * mU | | OXOXO OOOOO XX |
| WV- | T * fA * mCfA * mGfU * mAfG * mAfU * mGfA * mGfG * | TACAGUAGAUGAGGGAGCAGGTU | XXOXO XOXOX |
| 5272 | mGfAmGfCmAfGmG * T * mU | | OXOXO OOOOO XX |
| WV- | T * fC * mAfC * mAfG * mUfA * mGfA * mUfG * mAfG * | TCACAGUAGAUGAGGGAGCAGTU | XXOXO XOXOX |
| 5273 | mGfGmAfGmCfAmG * T * mU | | OXOXO OOOOO XX |
| WV- | T * fA * mCfA * mCfA * mGfU * mAfG * mAfU * mGfA * | TACACAGUAGAUGAGGGAGCATU | XXOXO XOXOX |
| 5274 | mGfGmGfAmGfCmA * T * mU | | OXOXO OOOOO XX |
| WV- | T * fC * mAfC * mAfC * mAfG * mUfA * mGfA * mUfG * | TCACACAGUAGAUGAGGGAGCTU | XXOXO XOXOX |
| 5275 | mAfGmGfGmAfGmC * T * mU | | OXOXO OOOOO XX |
| WV- | T * fG * mCfA * mCfA * mCfA * mGfU * mAfG * mAfU * | TGCACACAGUAGAUGAGGGAGTU | XXOXO XOXOX |
| 5276 | mGfAmGfGmGfAmG * T * mU | | OXOXO OOOOO XX |
| WV- | T * fU * mGfC * mAfC * mAfC * mAfG * mUfA * mGfA * | TUGCACACAGUAGAUGAGGGATU | XXOXO XOXOX |
| 5277 | mUfGmAfGmGfGmA * T * mU | | OXOXO OOOOO XX |
| WV- | T * fG * mUfG * mCfA * mCfA * mCfA * mGfU * mAfG * | TGUGCACACAGUAGAUGAGGGTU | XXOXO XOXOX |
| 5278 | mAfUmGfAmGfGmG * T * mU | | OXOXO OOOOO XX |
| WV- | T * fA * mGfU * mGfC * mAfC * mAfC * mAfG * mUfA * | TAGUGCACACAGUAGAUGAGGTU | XXOXO XOXOX |
| 5279 | mGfAmUfGmAfGmG * T * mU | | OXOXO OOOOO XX |
| WV- | T * fA * mAfG * mUfG * mCfA * mCfA * mCfA * mGfU * | TAAGUGCACACAGUAGAUGAGTU | XXOXO XOXOX |
| 5280 | mAfGmAfUmGfAmG * T * mU | | OXOXO OOOOO XX |
| WV- | T * fG * mAfA * mGfU * mGfC * mAfC * mAfC * mAfG * | TGAAGUGCACACAGUAGAUGATU | XXOXO XOXOX |
| 5281 | mUfAmGfAmUfGmA * T * mU | | OXOXO OOOOO XX |
| WV- | T * fU * mGfA * mAfG * mUfG * mCfA * mCfA * mCfA * | TUGAAGUGCACACAGUAGAUGTU | XXOXO XOXOX |
| 5282 | mGfUmAfGmAfUmG * T * mU | | OXOXO OOOOO XX |
| WV- | T * fA * mUfG * mAfA * mGfU * mGfC * mAfC * mAfC * | TAUGAAGUGCACACAGUAGAUTU | XXOXO XOXOX |
| 5283 | mAfGmUfAmGfAmU * T * mU | | OXOXO OOOOO XX |
| WV- | T * fG * mAfU * mGfA * mAfG * mUfG * mCfA * mCfA * | TGAUGAAGUGCACACAGUAGATU | XXOXO XOXOX |
| 5284 | mCfAmGfUmAfGmA * T * mU | | OXOXO OOOOO XX |
| WV- | T * fG * mGfA * mUfG * mAfA * mGfU * mGfC * mAfC * | TGGAUGAAGUGCACACAGUAGTU | XXOXO XOXOX |
| 5285 | mAfCmAfGmUfAmG * T * mU | | OXOXO OOOOO XX |
| WV- | T * fA * mGfG * mAfU * mGfA * mAfG * mUfG * mCfA * | TAGGAUGAAGUGCACACAGUATU | XXOXO XOXOX |
| 5286 | mCfAmCfAmGfUmA * T * mU | | OXOXO OOOOO XX |
| WV- | mGmCmAmC * SA * SA * SG * SG * SG * SC * SA * SC * | GCACAAGGGCA CAGACUUC | OOOSS SSSSS |
| 6013 | RA * SG * SmAmCmUmU * SmC | | SRSSOOOS |
| WV- | mCmAmC * SA * SA * SG * SG * SG * SC * SA * SC * RA * | CACAAGGGCACAGACUUC | OOSSS SSSSS |
| 6014 | SG * SmAmCmUmU * SmC | | RSSOOOS |
| WV- | mUmGmCmA * SC * SA * SC * SA * SG * ST * SA * SG * | UGCACACAGTAGATGAGGG | OOOSS SSSSS |
| 6015 | RA * ST * SmGmAmGmG * SmG | | SRSSOOOS |
| WV- | mGmCmA * SC * SA * SC * SA * SG * ST * SA * SG * RA * | GCACACAGTAGATGAGGG | OOSSS SSSSS |
| 6016 | ST * SmGmAmGmG * SmG | | RSSOOOS |
| WV- | G * SmGmCmAmC * SA * SA * SG * SG * SG * SC * SA * | GGCACAAGGGCA CAGACUUC | SOOOS SSSSS SSSRS |
| 6506 | SC * SA * RG * SmAmCmUmU * SC | | OOOS |
| WV- | mG * SGeom5CeoAeomC * SA * SA * SG * SG * SG * SC * | GGCACAAGGGCA CAGACUUC | SOOOS SSSSS SSRSS |
| 8706 | SA * SC * RA * SG * SmAmCmUmU * SmC | | OOOS |
| WV- | mG * SGeom5CeoAeomC * SA * SA * SG * SG * SG * SC * | GGCACAAGGGCA CAGACUUC | SOOOS SSSSS SSRSS |
| 8707 | SA * SC * RA * SG * SmA * SmC * SmU * SmU * SmC | | SSSS |
| WV- | mG * Geom5CeoAeomC * A * A * G * G * G * C * A * C * A | GGCACAAGGGCA CAGACUUC | XOOOX XXXXX |
| 8708 | * G * mAmCmUmU * mC | | XXXXX OOOX |
| WV- | mG * Geom5CeoAeomC * A * A * G * G * G * C * A * C * A | GGCACAAGGGCA CAGACUUC | XOOOX XXXXX |
| 8709 | * G * mA * mC * mU * mU * mC | | XXXXX XXXX |
| WV- | mG * STeoGeom5CeomA * SC * SA * SC * SA * SG * ST * | GTGCACACAGTAGATGAGGG | SOOOS SSSSS SSRSS |
| 8710 | SA * SG * RA * ST * SmGmAmGmG * SmG | | OOOS |
| WV- | mG * STeoGeom5CeomA * SC * SA * SC * SA * SG * ST * | GTGCACACAGTAGATGAGGG | SOOOS SSSSS SSRSS |
| 8711 | SA * SG * RA * ST * SmG * SmA * SmG * SmG * SmG | | SSSS |
| WV- | mG * TeoGeom5CeomA * C * A * C * A * G * T * A * G * A | GTGCACACAGTAGATGAGGG | XOOOX XXXXX |
| 8712 | * T * mGmAmGmG * mG | | XXXXX OOOX |
| WV- | mG * TeoGeom5CeomA * C * A * C * A * G * T * A * G * A | GTGCACACAGTAGATGAGGG | XOOOX XXXXX |
| 8713 | * T * mG * mA * mG * mG * mG | | XXXXX XXXX |
| WV- | mA * GeoGeoGeomC * G * C * A * G * A * C * T * T * C * C | AGGGCGCAGACTTCCAAAGG | XOOOX XXXXX |
| 9660 | * mA * mA * mA * mG * mG | | XXXXX XXXX |
| WV- | mA * AeoGeoGeomG * C * G * C * A * G * A * C * T * T * C | AAGGGCGCAGACTTCCAAAG | XOOOX XXXXX |
| 9661 | *mC * mA * mA * mA * mG | | XXXXX XXXX |
| WV- | mC * AeoAeoGeomG * G * C * G * C * A * G * A * C * T * T | CAAGGGCGCAGACTTCCAAA | XOOOX XXXXX |
| 9662 | * mC * mC * mA * mA * mA | | XXXXX XXXX |
| WV- | mA * m5CeoAeoAeomG * G * G * C * G * C * A * G * A * C | ACAAGGGCGCAGACTUCCAA | XOOOX XXXXX |
| 9663 | * T * mU * mC * mC * mA * mA | | XXXXX XXXX |
| WV- | mC * Aeom5CeoAeomA * G * G * G * C * G * C * A * G * A | CACAAGGGCGCAGACUUCCA | XOOOX XXXXX |
| 9664 | * C * mU * mU * mC * mC * mA | | XXXXX XXXX |
| WV- | mG * m5CeoAeom5CeomA * A * G * G * G * C * G * C * A * | GCACAAGGGCGCAGACUUCC | XOOOX XXXXX |
| 9665 | G * A * mC * mU * mU * mC * mC | | XXXXX XXXX |
| WV- | mG * Geom5CeoAeomC * A * A * G * G * G * C * G * C * A | GGCACAAGGGCGCAGACUUC | XOOOX XXXXX |
| 9666 | * G * mA * mC * mU * mU * mC | | XXXXX XXXX |
| WV- | mG * GeoGeom5CeomA * C * A * A * G * G * G * C * G * C | GGGCACAAGGGCGCAGACUU | XOOOX XXXXX |
| 9667 | * A * mG * mA * mC * mU * mU | | XXXXX XXXX |
| WV- | mA * GeoGeoGeomC * A * C * A * A * G * G * G * C * G * | AGGGCACAAGGGCGCAGACU | XOOOX XXXXX |
| 9668 | C * mA * mG * mA * mC * mU | | XXXXX XXXX |
| WV- | mC * AeoGeoGeomG * C * A * C * A * A * G * G * G * C * | CAGGGCACAAGGGCGCAGAC | XOOOX XXXXX |
| 9669 | G * mC * mA * mG * mA * mC | | XXXXX XXXX |
| WV- | m5Ceo * Teom5CeoAeoGeo * T * A * A * C * A * T * T * G * | CTCAGTAACATTGACACCAC | XOOOX XXXXX |
| 9679 | A * C * Aeom5Ceom5CeoAeo * m5Ceo | | XXXXX OOOX |
| WV- | mG * mGmCmAmC * A * A * G * G * G * C * G * C * A * G | GGCACAAGGGCGCAGACUUC | XOOOX XXXXX |
| 9692 | * mAmCmUmU * mC | | XXXXX OOOX |
| WV- | mG * SmGmCmAmC * SA * SA * SG * SG * SG * SC * SG * | GGCACAAGGGCGCAGACUUC | SOOOS SSSSS SSRSS |
| 9693 | SC * RA * SG * SmAmCmUmU * SmC | | OOOS |
| WV- | L001mG * SmGmCmAmC * SA * SA * SG * SG * SG * SC * | GGCACAAGGGCA CAGACUUC | OSOOO SSSSS SSSRS |
| 9854 | SA * SC * RA * SG * SmAmCmUmU * SmC | | SOOOS |
| WV- | mG * SmGmCmAmC * SA * SA * SG * SG * SG * SC * SA * | GGCACAAGGGCA CAGACUUC | SOOOS SSSSS SSRSS |
| 9855 | SC * RA * SG * SmAmCmUmU * SmCL004 | | OOOS |
| WV- | L001mG * SmUmGmCmA * SC * SA * SC * SA * SG * ST * | GUGCACACAGTAGATGAGGG | OSOOO SSSSS SSSRS |
| 9856 | SA * SG * RA * ST * SmGmAmGmG * SmG | | SOOOS |
| WV- | mG * SmUmGmCmA * SC * SA * SC * SA * SG * ST * SA * | GUGCACACAGTAGATGAGGG | SOOOS SSSSS SSRSS |
| 9857 | SG * RA * ST * SmGmAmGmG * SmGL004 | | OOOS |
| WV- | POT * fA * mCfA * mGfA * mCfU * mUfC * mCfA * mAfA * | TACAGACUUCCAAAGGCUCCGTU | XXOXO XOXOX |
| 10107 | mG * fG * mC * fU * mC * fC * mG * T * mU | | OXOXX XXXXX XX |
| WV- | POT * fC * mAfC * mAfG * mAfC * mUfU * mCfC * mAfA * | TCACAGACUUCCAAAGGCUCCTU | XXOXO XOXOX |
| 10108 | mA * fG * mG * fC * mU * fC * mC * T * mU | | OXOXX XXXXX XX |
| WV- | POT * fG * mCfA * mCfA * mGfA * mCfU * mUfC * mCfA * | TGCACAGACUUCCAAAGGCUCTU | XXOXO XOXOX |
| 10109 | mA * fA * mG * fG * mC * fU * mC * T * mU | | OXOXX XXXXX XX |
| WV- | POT * fG * mGfC * mAfC * mAfG * mAfC * mUfU * mCfC * | TGGCACAGACUUCCAAAGGCUTU | XXOXO XOXOX |
| 10110 | mA * fA * mA * fG * mG * fC * mU * T * mU | | OXOXX XXXXX XX |
| WV- | POT * fG * mGfG * mCfA * mCfA * mGfA * mCfU * mUfC * | TGGGCACAGACUUCCAAAGGCTU | XXOXO XOXOX |
| 10111 | mC * fA * mA * fA * mG * fG * mC * T * mU | | OXOXX XXXXX XX |
| WV- | POT * fA * mGfG * mGfC * mAfC * mAfG * mAfC * mUfU * | TAGGGCACAGACUUCCAAAGGTU | XXOXO XOXOX |
| 10112 | mC * fC * mA * fA * mA * fG * mG * T * mU | | OXOXX XXXXX XX |
| WV- | POT * fA * mAfG * mGfG * mCfA * mCfA * mGfA * mCfU * | TAAGGGCACAGACUUCCAAAGTU | XXOXO XOXOX |
| 10113 | mU * fC * mC * fA * mA * fA * mG * T * mU | | OXOXX XXXXX XX |
| WV- | POT * fC * mAfA * mGfG * mGfC * mAfC * mAfG * mAfC * | TCAAGGGCACAGACUUCCAAATU | XXOXO XOXOX |
| 10114 | mU * fU * mC * fC * mA * fA * mA * T * mU | | OXOXX XXXXX XX |
| WV- | POT * fA * mCfA * mAfG * mGfG * mCfA * mCfA * mGfA * | TACAAGGGCACAGACUUCCAATU | XXOXO XOXOX |
| 10115 | mC * fU * mU * fC * mC * fA * mA * T * mU | | OXOXX XXXXX XX |
| WV- | POT * fC * mAfC * mAfA * mGfG * mGfC * mAfC * mAfG * | TCACAAGGGCACAGACUUCCATU | XXOXO XOXOX |
| 10116 | mA * fC * mU * fU * mC * fC * mA * T * mU | | OXOXX XXXXX XX |
| WV- | POT * fG * mCfA * mCfA * mAfG * mGfG * mCfA * mCfA * | TGCACAAGGGCA | XXOXO XOXOX |
| 10117 | mG * fA * mC * fU * mU * fC * mC * T * mU | CAGACUUCCTU | OXOXX XXXXX XX |
| WV- | POT * fG * mGfC * mAfC * mAfA * mGfG * mGfC * mAfC * | TGGCACAAGGGCA | XXOXO XOXOX |
| 10118 | mA * fG * mA * fC * mU * fU * mC * T * mU | CAGACUUCTU | OXOXX XXXXX XX |
| WV- | POT * fG * mGfG * mCfA * mCfA * mAfG * mGfG * mCfA * | TGGGCACAAGGGCA | XXOXO XOXOX |
| 10119 | mC * fA * mG * fA * mC * fU * mU * T * mU | CAGACUUTU | OXOXX XXXXX XX |
| WV- | POT * fA * mGfG * mGfC * mAfC * mAfA * mGfG * mGfC * | TAGGGCACAAGGGCA | XXOXO XOXOX |
| 10120 | mA * fC * mA * fG * mA * fC * mU * T * mU | CAGACUTU | OXOXX XXXXX XX |
| WV- | POT * fC * mAfG * mGfG * mCfA * mCfA * mAfG * mGfG * | TCAGGGCACAAGGGCA | XXOXO XOXOX |
| 10121 | mC * fA * mC * fA * mG * fA * mC * T * mU | CAGACTU | OXOXX XXXXX XX |
| WV- | POT * fG * mCfA * mGfG * mGfC * mAfC * mAfA * mGfG * | TGCAGGGCACAAGGGCA | XXOXO XOXOX |
| 10122 | mG * fC * mA * fC * mA * fG * mA * T * mU | CAGATU | OXOXX XXXXX XX |
| WV- | POT * fG * mGfC * mAfG * mGfG * mCfA * mCfA * mAfG * | TGGCAGGGCACAAGGGCA | XXOXO XOXOX |
| 10123 | mG * fG * mC * fA * mC * fA * mG * T * mU | CAGTU | OXOXX XXXXX XX |
| WV- | POT * fA * mGfG * mCfA * mGfG * mGfC * mAfC * mAfA * | TAGGCAGGGCACAAGGGCA | XXOXO XOXOX |
| 10124 | mG * fG * mG * fC * mA * fC * mA * T * mU | CATU | OXOXX XXXXX XX |
| WV- | POT * fG * mAfG * mGfC * mAfG * mGfG * mCfA * mCfA * | TGAGGCAGGGCACAAGGGCA | XXOXO XOXOX |
| 10125 | mA * fG * mG * fG * mC * fA * mC * T * mU | CTU | OXOXX XXXXX XX |
| WV- | POT * fG * mGfA * mGfG * mCfA * mGfG * mGfC * mAfC * | TGGAGGCAGGGCACAAGGGCA | XXOXO XOXOX |
| 10126 | mA * fA * mG * fG * mG * fC * mA * T * mU | TU | OXOXX XXXXX XX |
| WV- | POT * fA * mCfA * mGfA * mCfU * mUfC * mCfA * mAfA * | TACAGACUUCCAAAGGCUCCGTU | XXOXO XOXOX |
| 10127 | mGfGmCfUmCfCmG * T * mU | | OXOXO OOOOO XX |
| WV- | POT * fC * mAfC * mAfG * mAfC * mUfU * mCfC * mAfA * | TCACAGACUUCCAAAGGCUCCTU | XXOXO XOXOX |
| 10128 | mAfGmGfCmUfCmC * T * mU | | OXOXO OOOOO XX |
| WV- | POT * fG * mCfA * mCfA * mGfA * mCfU * mUfC * mCfA * | TGCACAGACUUCCAAAGGCUCTU | XXOXO XOXOX |
| 10129 | mAfAmGfGmCfUmC * T * mU | | OXOXO OOOOO XX |
| WV- | POT * fG * mGfC * mAfC * mAfG * mAfC * mUfU * mCfC * | TGGCACAGACUUCCAAAGGCUTU | XXOXO XOXOX |
| 10130 | mAfAmAfGmGfCmU * T * mU | | OXOXO OOOOO XX |
| WV- | POT * fG * mGfG * mCfA * mCfA * mGfA * mCfU * mUfC * | TGGGCACAGACUUCCAAAGGCTU | XXOXO XOXOX |
| 10131 | mCfAmAfAmGfGmC * T * mU | | OXOXO OOOOO XX |
| WV- | POT * fA * mGfG * mGfC * mAfC * mAfG * mAfC * mUfU * | TAGGGCACAGACUUCCAAAGGTU | XXOXO XOXOX |
| 10132 | mCfCmAfAmAfGmG * T * mU | | OXOXO OOOOO XX |
| WV- | POT * fA * mAfG * mGfG * mCfA * mCfA * mGfA * mCfU * | TAAGGGCACAGACUUCCAAAGTU | XXOXO XOXOX |
| 10133 | mUfCmCfAmAfAmG * T * mU | | OXOXO OOOOO XX |
| WV- | POT * fC * mAfA * mGfG * mGfC * mAfC * mAfG * mAfC * | TCAAGGGCACAGACUUCCAAATU | XXOXO XOXOX |
| 10134 | mUfUmCfCmAfAmA * T * mU | | OXOXO OOOOO XX |
| WV- | POT * fA * mCfA * mAfG * mGfG * mCfA * mCfA * mGfA * | TACAAGGGCACAGACUUCCAATU | XXOXO XOXOX |
| 10135 | mCfUmUfCmCfAmA * T * mU | | OXOXO OOOOO XX |
| WV- | POT * fC * mAfC * mAfA * mGfG * mGfC * mAfC * mAfG * | TCACAAGGGCACAGACUUCCATU | XXOXO XOXOX |
| 10136 | mAfCmUfUmCfCmA * T * mU | | OXOXO OOOOO XX |
| WV- | POT * fG * mCfA * mCfA * mAfG * mGfG * mCfA * mCfA * | TGCACAAGGGCA | XXOXO XOXOX |
| 10137 | mGfAmCfUmUfCmC * T * mU | CAGACUUCCTU | OXOXO OOOOO XX |
| WV- | POT * fG * mGfC * mAfC * mAfA * mGfG * mGfC * mAfC * | TGGCACAAGGGCA | XXOXO XOXOX |
| 10138 | mAfGmAfCmUfUmC * T * mU | CAGACUUCTU | OXOXO OOOOO XX |
| WV- | POT * fG * mGfG * mCfA * mCfA * mAfG * mGfG * mCfA * | TGGGCACAAGGGCA | XXOXO XOXOX |
| 10139 | mCfAmGfAmCfUmU * T * mU | CAGACUUTU | OXOXO OOOOO XX |
| WV- | POT * fA * mGfG * mGfC * mAfC * mAfA * mGfG * mGfC * | TAGGGCACAAGGGCA | XXOXO XOXOX |
| 10140 | mAfCmAfGmAfCmU * T * mU | CAGACUTU | OXOXO OOOOO XX |
| WV- | POT * fC * mAfG * mGfG * mCfA * mCfA * mAfG * mGfG * | TCAGGGCACAAGGGCA | XXOXO XOXOX |
| 10141 | mCfAmCfAmGfAmC * T * mU | CAGACTU | OXOXO OOOOO XX |
| WV- | POT * fG * mCfA * mGfG * mGfC * mAfC * mAfA * mGfG * | TGCAGGGCACAAGGGCA | XXOXO XOXOX |
| 10142 | mGfCmAfCmAfGmA * T * mU | CAGATU | OXOXO OOOOO XX |
| WV- | POT * fG * mGfC * mAfG * mGfG * mCfA * mCfA * mAfG * | TGGCAGGGCACAAGGGCA | XXOXO XOXOX |
| 10143 | mGfGmCfAmCfAmG * T * mU | CAGTU | OXOXO OOOOO XX |
| WV- | POT * fA * mGfG * mCfA * mGfG * mGfC * mAfC * mAfA * | TAGGCAGGGCACAAGGGCA | XXOXO XOXOX |
| 10144 | mGfGmGfCmAfCmA * T * mU | CATU | OXOXO OOOOO XX |
| WV- | POT * fG * mAfG * mGfC * mAfG * mGfG * mCfA * mCfA * | TGAGGCAGGGCACAAGGGCA | XXOXO XOXOX |
| 10145 | mAfGmGfGmCfAmC * T * mU | CTU | OXOXO OOOOO XX |
| WV- | POT * fG * mGfA * mGfG * mCfA * mGfG * mGfC * mAfC * | TGGAGGCAGGGCACAAGGGCA | XXOXO XOXOX |
| 10146 | mAfAmGfGmGfCmA * T * mU | TU | OXOXO OOOOO XX |
| WV- | fA *fA *fC *fAfAfGfAfUfGfA fAfG rA rG rC * rA rC rC rA rA | AACAAGAUGA AGAGCACCAA | XXXOOOOOOO |
| 10213 | rG rU rU rU rU rA rG rA rG rC rU rA rU * mG * mC * mU | GUUUUAGAGCU AUGCU | OOOOXOOOOO |
| | | | OOOOOOOOOO |
| | | | OOXXX |
| WV- | Mod012L001mG * SmGmCmAmC * SA * SA * SG * SG * SG | GGCACAAGGGCA CAGACUUC | OSOOO SSSSS |
| 10483 | * SC * SA * SC * RA * SG * SmAmCmUmU * SmC | | SSSRS SOOOS |
| WV- | Mod085L001mG * SmGmCmAmC * SA * SA * SG * SG * SG | GGCACAAGGGCA CAGACUUC | OSOOO SSSSS |
| 10484 | * SC * SA * SC * RA * SG * SmAmCmUmU * SmC | | SSSRS SOOOS |
| WV- | mG * SmGmCmAmC * SA * SA * SG * SG * SG * SC * SA * | GGCACAAGGGCA CAGACUUC | SOOOS SSSSS SSRSS |
| 10485 | SC * RA * SG * SmAmCmUmU * SmCL004Mod012 | | OOOS |
| WV- | Mod086L001mG * SmGmCmAmC * SA * SA * SG * SG * SG | GGCACAAGGGCA CAGACUUC | OSOOO SSSSS |
| 10486 | * SC * SA * SC * RA * SG * SmAmCmUmU * SmC | | SSSRS SOOOS |
| WV- | Mod012L001mG * SmUmGmCmA * SC * SA * SC * SA * SG | GUGCACACAGTAGATGAGGG | OSOOO SSSSS |
| 10631 | * ST * SA * SG * RA * ST * SmGmAmGmG * SmG | | SSSRS SOOOS |
| WV- | Mod085L001mG * SmUmGmCmA * SC * SA * SC * SA * SG | GUGCACACAGTAGATGAGGG | OSOOO SSSSS |
| 10632 | * ST * SA * SG * RA * ST * SmGmAmGmG * SmG | | SSSRS SOOOS |
| WV- | mG * SmUmGmCmA * SC * SA * SC * SA * SG * ST * SA * | GUGCACACAGTAGATGAGGG | SOOOS SSSSS SSRSS |
| 10633 | SG * RA * ST * SmGmAmGmG * SmGL004Mod012 | | OOOS |
| WV- | mG * SmGmCmAmC * SA * SA * SG * SG * SG * SC * SA * | GGCACAAGGGCA CAGACUUC | SOOOS SSSSS SSRSS |
| 10640 | SC * RA * SG * SmAmCmUmU * SmCL004Mod085 | | OOOS |
| WV- | mG * SmGmCmAmC * SA * SA * SG * SG * SG * SC * SA * | GGCACAAGGGCA CAGACUUC | SOOOS SSSSS SSRSS |
| 10641 | SC * RA * SG * SmAmCmUmU * SmCL004Mod086 | | OOOS |
| WV- | Mod086L001mG * SmUmGmCmA * SC * SA * SC * SA * SG | GUGCACACAGTAGATGAGGG | OSOOO SSSSS |
| 10642 | * ST * SA * SG * RA * ST * SmGmAmGmG * SmG | | SSSRS SOOOS |
| WV- | mG * SmUmGmCmA * SC * SA * SC * SA * SG * ST * SA * | GUGCACACAGTAGATGAGGG | SOOOS SSSSS SSRSS |
| 10643 | SG * RA * ST * SmGmAmGmG * SmGL004Mod085 | | OOOS |
| WV- | mG * SmUmGmCmA * SC * SA * SC * SA * SG * ST * SA * | GUGCACACAGTAGATGAGGG | SOOOS SSSSS SSRSS |
| 10644 | SG * RA * ST * SmGmAmGmG * SmGL004Mod086 | | OOOS |
| WV- | rG rC rC rU rU rU rG rG rA rA rG rU rC rU rG rC rG rC rC rC | GCCUUUGGAAGUCUGCGCCC | OOOOO OOOOO |
| 10765 | rU rU rG rU rG rC rC rC rU rG rC | UUGUGCCCUGC | OOOOO OOOOO |
| | | | OOOOO OOOOO |
| WV- | rG rU rG rG rU rG rU rC rA rA rU rG rU rU rA rC rU rG rA rG | GUGGUGUCAAUGUUACUGAG | OOOOO OOOOO |
| 10766 | | | OOOOO OOOO |
| WV- | mA * SGeoGeoGeomC * SG * SC * RA * SG * SA * SC * ST * | AGGGCGCAGACTTCCAAAGG | SOOOS SRSSS SSSSS |
| 10767 | ST * SC * SC * SmA * SmA * SmA * SmG * SmG | | SSSS |
| WV- | mA * SAeoGeoGeomG * SC * SG * SC * RA * SG * SA * SC | AAGGGCGCAGACTTCCAAAG | SOOOS SSRSS SSSSS |
| 10768 | * ST * ST * SC * SmC * SmA * SmA * SmA * SmG | | SSSS |
| WV- | mC * SAeoAeoGeomG * SG * SC * SG * SC * RA * SG * SA | CAAGGGCGCAGACTTCCAAA | SOOOS SSSRS SSSSS |
| 10769 | * SC * ST * ST * SmC * SmC * SmA * SmA * SmA | | SSSS |
| WV- | mA * Sm5CeoAeoAeomG * SG * SG * SC * SG * SC * RA * | ACAAGGGCGCAGACTUCCAA | SOOOS SSSSR SSSSS |
| 10770 | SG * SA * SC * ST * SmU * SmC * SmC * SmA * SmA | | SSSS |
| WV- | mC * SAeom5CeoAeomA * SG * SG * SG * SC * SG * SC * | CACAAGGGCGCAGACUUCCA | SOOOS SSSSS RSSSS |
| 10771 | RA * SG * SA * SC * SmU * SmU * SmC * SmC * SmA | | SSSS |
| WV- | mG * Sm5CeoAeom5CeomA * SA * SG * SG * SG * SC * SG | GCACAAGGGCG CAGACUUCC | SOOOS SSSSS SRSSS |
| 10772 | * SC * RA * SG * SA * SmC * SmU * SmU * SmC * SmC | | SSSS |
| WV- | mG * SGeom5CeoAeomC * SA * SA * SG * SG * SG * SC * | GGCACAAGGGCGCAGACUUC | SOOOS SSSSS SSRSS |
| 10773 | SG * SC * RA * SG * SmA * SmC * SmU * SmU * SmC | | SSSS |
| WV- | mG * SGeoGeom5CeomA * SC * SA * SA * SG * SG * SG * | GGGCACAAGGGCGCAGACUU | SOOOS SSSSS SSSRS |
| 10774 | SC * SG * SC * RA * SmG * SmA * SmC * SmU * SmU | | SSSS |
| WV- | mA * SGeoGeoGeomC * SA * SC * SA * SA * SG * SG * SG | AGGGCACAAGGGCGCAGACU | SOOOS SSSSS SSSSR |
| 10775 | * SC * SG * SC * RmA * SmG * SmA * SmC * SmU | | SSSS |
| WV- | mC * SAeoGeoGeomG * SC * SA * SC * SA * SA * SG * SG * | CAGGGCACAAGGGCGCAGAC | SOOOS SSSSS SSSSS |
| 10776 | SG * SC * SG * SmC * RmA * SmG * SmA * SmC | | RSSS |
| WV- | mC * m5Ceom5CeoGeomC * C * G * T * A * G * C * C * T * | CCCGCCGTAGCCTGGGACCC | XOOOX XXXXX |
| 10783 | G * G * mG * mA * mC * mC * mC | | XXXXX XXXX |
| WV- | mC * m5Ceom5Ceom5CeomG * C * C * G * T * A * G * C * C | CCCCGCCGTAGCCTGGGACC | XOOOX XXXXX |
| 10784 | * T * G * mG * mG * mA * mC * mC | | XXXXX XXXX |
| WV- | mC * AeoTeom5CeomC * C * C * G * C * C * G * T * A * G * | CATCCCCGCCGTAGCCUGGG | XOOOX XXXXX |
| 10785 | C * mC * mU * mG * mG * mG | | XXXXX XXXX |
| WV- | mC * m5CeoGeom5CeomC * A * T * C * C * C * C * G * C * | CCGCCATCCCCGCCGUAGCC | XOOOX XXXXX |
| 10786 | C * G * mU * mA * mG * mc* mC | | XXXXX XXXX |
| WV- | mA * m5Ceom5CeoGeomC * C * A * T * C * C * C * C * G * | ACCGCCATCCCCGCCGUAGC | XOOOX XXXXX |
| 10787 | C * C * mG * mU * mA * mG * mC | | XXXXX XXXX |
| WV- | mU * Aeom5Ceom5CeomG * C * C * A * T * C * C * C * C * | UACCGCCATCCCCGCCGUAG | XOOOX XXXXX |
| 10788 | G * C * mC * mG * mU * mA * mG | | XXXXX XXXX |
| WV- | mU * TeoAeom5CeomC * G * C * C * A * T * C * C * C * C * | UTACCGCCATCCCCGCCGUA | XOOOX XXXXX |
| 10789 | G * mC * mC * mG * mU * mA | | XXXXX XXXX |
| WV- | mG * TeoTeoAeomC * C * G * C * C * A * T * C * C * C * C | GTTACCGCCATCCCCGCCGU | XOOOX XXXXX |
| 10790 | * mG * mC * mC * mG * mU | | XXXXX XXXX |
| WV- | mG * GeoTeoTeomA * C * C * G * C * C * A * T * C * C * C | GGTTACCGCCATCCCCGCCG | XOOOX XXXXX |
| 10791 | * mC * mG * mC * mC * mG | | XXXXX XXXX |
| WV- | mA * GeoGeoGeomU * T * A * C * C * G * C * C * A * T * C | AGGGUTACCGCCATCCCCGC | XOOOX XXXXX |
| 10792 | * mC * mC * mC * mG * mC | | XXXXX XXXX |
| WV- | mC * AeoGeoGeomG * T * T * A * C * C * G * C * C * A * T | CAGGGTTACCGCCATCCCCG | XOOOX XXXXX |
| 10793 | * mC * mC * mC * mC * mG | | XXXXX XXXX |
| WV- | mG * m5CeoAeoGeomG * G * T * T * A * C * C * G * C * C * | GCAGGGTTACCGCCAUCCCC | XOOOX XXXXX |
| 10794 | A * mU * mC * mC * mC * mC | | XXXXX XXXX |
| WV- | mU * Geom5CeoAeomG * G * G * T * T * A * C * C * G * C * | UGCAGGGTTACCGCCAUCCC | XOOOX XXXXX |
| 10795 | C * mA * mU * mC * mC * mC | | XXXXX XXXX |
| WV- | mA * Geom5Ceom5CeomG * G * G * G * G * T * T * C * G * | AGCCGGGGGTTCGTGUCGCC | XOOOX XXXXX |
| 10796 | T * G * mU * mC * mG * mC * mC | | XXXXX XXXX |
| WV- | mG * AeoGeom5CeomC * G * G * G * G * G * T * T * C * G | GAGCCGGGGGTTCGTGUCGC | XOOOX XXXXX |
| 10797 | * T * mG * mU * mC * mG * mC | | XXXXX XXXX |
| WV- | mG * GeoAeoGeomC * C * G * G * G * G * G * T * T * C * G | GGAGCCGGGGGTTCGUGUCG | XOOOX XXXXX |
| 10798 | * mU * mG * mU * mC * mG | | XXXXX XXXX |
| WV- | mC * GeoGeoAeomG * C * C * G * G * G * G * G * T * T * C | CGGAGCCGGGGGTTCGUGUC | XOOOX XXXXX |
| 10799 | * mG * mU * mG * mU * mC | | XXXXX XXXX |
| WV- | mC * m5Ceom5CeoTeomC * A * T * G * G * G * C * T * C * | CCCTCATGGGCTCTGGGUUG | XOOOX XXXXX |
| 10800 | T * G * mG * mG * mU * mU * mG | | XXXXX XXXX |
| WV- | mU * m5Ceom5Ceom5CeomU * C * A * T * G * G * G * C * T | UCCCUCATGGGCTCTGGGUU | XOOOX XXXXX |
| 10801 | * C * T * m G * mG * mG * mU * mU | | XXXXX XXXX |
| WV- | mC * AeoGeoGeomA * G * G * G * G * G * C * G * G * G * T | CAGGAGGGGGCGGGTGUCCC | XOOOX XXXXX |
| 10802 | * mG * mU * mC * mC * mC | | XXXXX XXXX |
| WV- | mC * m5CeoAeoGeomG * A * G * G * G * G * G * C * G * G | CCAGGAGGGGGCGGGUGUCC | XOOOX XXXXX |
| 10803 | * G * mU * mG * mU * mC * mC | | XXXXX XXXX |
| WV- | mC * m5Ceom5CeoAeomA * G * T * G * A * G * G * G * A * | CCCAAGTGAGGGAGCGGGGC | XOOOX XXXXX |
| 10804 | G * C * mG * mG * mG * mG * mC | | XXXXX XXXX |
| WV- | mA * GeoGeom5CeomC * C * C * A * A * C * A * A * G * G | AGGCCCCAACAAGGCUCUGC | XOOOX XXXXX |
| 10805 | * C * mU * mC * mU * mG * mC | | XXXXX XXXX |
| WV- | mG * Geom5CeoTeomC * T * G * G * G * T * T * G * C * T * | GGCTCTGGGTTGCTGGGUCA | XOOOX XXXXX |
| 10806 | G * mG * mG * mU * mC * mA | | XXXXX XXXX |
| WV- | mG * Teom5Ceom5CeomC * T * C * A * T * G * G * G * C * | GTCCCTCATGGGCTCUGGGU | XOOOX XXXXX |
| 10807 | T * C * mU * mG * mG * mG * mU | | XXXXX XXXX |
| WV- | mG * TeoGeoTeomC * C * C * T * C * A * T * G * G * G * C | GTGTCCCTCATGGGCUCUGG | XOOOX XXXXX |
| 10808 | * mU * mC * mU * mG * mG | | XXXXX XXXX |
| WV- | mC * m5CeoAeoTeomC * C * C * C * G * C * C * G * T * A * | CCATCCCCGCCGTAGCCUGG | XOOOX XXXXX |
| 10809 | G * mC * mC * mU * mG * mG | | XXXXX XXXX |
| WV- | mG * m5Ceom5CeoAeomU * C * C * C * C * G * C * C * G * | GCCAUCCCCGCCGTAGCCUG | XOOOX XXXXX |
| 10810 | T * A * mG * mC * mC * mU * mG | | XXXXX XXXX |
| WV- | mC * Geom5Ceom5CeomA * T * C * C * C * C * G * C * C * | CGCCATCCCCGCCGTAGCCU | XOOOX XXXXX |
| 10811 | G * T * mA * mG * mC * mC * mU | | XXXXX XXXX |
| WV- | mG * GeoGeoTeomU * A * C * C * G * C * C * A * T * C * C | GGGTUACCGCCATCCCCGCC | XOOOX XXXXX |
| 10812 | * mC * mC * mG * mC * mC | | XXXXX XXXX |
| WV- | mG * GeoGeom5CeomU * C * T * G * G * G * T * T * G * C * | GGGCUCTGGGTTGCTGGGUC | XOOOX XXXXX |
| 10813 | T * mG * mG * mG * mU * mC | | XXXXX XXXX |
| WV- | mA * GeoGeoAeomG * G * G * G * G * C * G * G * G * T * G | AGGAGGGGGCGGGTGUCCCU | XOOOX XXXXX |
| 10814 | * mU * mC * mC * mC * mU | | XXXXX XXXX |
| WV- | mG * GeoTeoGeomU * C * C * C * T * C * A * T * G * G * G | GGTGUCCCTCATGGGCUCUG | XOOOX XXXXX |
| 10815 | * mC * mU * mC * mU * mG | | XXXXX XXXX |
| WV- | mU * GeoTeom5CeomC * C * T * C * A * T * G * G * G * C * | UGTCCCTCATGGGCTCUGGG | XOOOX XXXXX |
| 10816 | T * mC * mU * mG * mG * mG | | XXXXX XXXX |
| WV- | mU * GeoGeoGeomC * T * C * T * G * G * G * T * T * G * C | UGGGCTCTGGGTTGCUGGGU | XOOOX XXXXX |
| 10817 | * mU * mG * mG * mG * mU | | XXXXX XXXX |
| WV- | mA * m5CeoAeoGeomU * G * T * T * G * G * C * C * A * T * | ACAGUGTTGGCCATGCCCAG | XOOOX XXXXX |
| 10818 | G * mC * mC * mC * mA * mG | | XXXXX XXXX |
| WV- | mA * m5Ceom5CeoGeomC * G * A * C * C * C * T * C * T * | ACCGCGACCCTCTGGACAGG | XOOOX XXXXX |
| 10819 | G * G * mA * mC * mA * mG * mG | | XXXXX XXXX |
| WV- | mU * Aeom5Ceom5CeomG * C * G * A * C * C * C * T * C * | UACCGCGACCCTCTGGACAG | XOOOX XXXXX |
| 10820 | T * G * mG * mA * mC * mA * mG | | XXXXX XXXX |
| WV- | mG * TeoAeom5CeomC * G * C * G * A * C * C * C * T * C * | GTACCGCGACCCTCTGGACA | XOOOX XXXXX |
| 10821 | T * mG * mG * mA * mC * mA | | XXXXX XXXX |
| WV- | mG * GeoTeoAeomC * C * G * C * G * A * C * C * C * T * C | GGTACCGCGACCCTCUGGAC | XOOOX XXXXX |
| 10822 | * mU * mG * mG * mA * mC | | XXXXX XXXX |
| WV- | mA * GeoGeoTeomA * C * C * G * C * G * A * C * C * C * T | AGGTACCGCGACCCTCUGGA | XOOOX XXXXX |
| 10823 | * mC * mU * mG * mG * mA | | XXXXX XXXX |
| WV- | mC * AeoGeoGeomG * A * G * G * T * A * C * C * G * C * G | CAGGGAGGTACCGCGACCCU | XOOOX XXXXX |
| 10824 | * mA * mC * mC * mC * mU | | XXXXX XXXX |
| WV- | mU * m5CeoAeoGeomG * G * A * G * G * T * A * C * C * G | UCAGGGAGGTACCGCGACCC | XOOOX XXXXX |
| 10825 | * C * mG * mA * mC * mC * mC | | XXXXX XXXX |
| WV- | mC * Teom5CeoAeomG * G * G * A * G * G * T * A * C * C * | CTCAGGGAGGTACCGCGACC | XOOOX XXXXX |
| 10826 | G * mC * mG * mA * mC * mC | | XXXXX XXXX |
| WV- | mC * m5CeoTeom5CeomA * G * G * G * A * G * G * T * A * | CCTCAGGGAGGTACCGCGAC | XOOOX XXXXX |
| 10827 | C * C * mG * mC * mG * mA * mC | | XXXXX XXXX |
| WV- | mG * GeoAeoAeomA * G * C * C * T * G * G * C * C * T * C | GGAAAGCCTGGCCTCAGGGA | XOOOX XXXXX |
| 10828 | * mA * mG * mG * mG * mA | | XXXXX XXXX |
| WV- | mA * TeoGeom5CeomC * C * A * G * C * A * C * G * C * A * | ATGCCCAGCACGCAGGCCAG | XOOOX XXXXX |
| 10829 | G * mG * mC * mC * mA * mG | | XXXXX XXXX |
| WV- | mG * m5Ceom5CeoAeomU * G * C * C * C * A * G * C * A * | GCCAUGCCCAGCACGCAGGC | XOOOX XXXXX |
| 10830 | C * G * mC * mA * mG * mG * mC | | XXXXX XXXX |
| WV- | mC * AeoGeom5CeomA * C * G * C * A * G * G * C * C * A | CAGCACGCAGGCCAGGGGCG | XOOOX XXXXX |
| 10831 | * G * mG * mG * mG * mC * mG | | XXXXX XXXX |
| WV- | mC * AeoTeoGeomC * C * C * A * G * C * A * C * G * C * A | CATGCCCAGCACGCAGGCCA | XOOOX XXXXX |
| 10832 | * mG * mG * mC * mC * mA | | XXXXX XXXX |
| WV- | mC * m5CeoAeoTeomG * C * C * C * A * G * C * A * C * G * | CCATGCCCAGCACGCAGGCC | XOOOX XXXXX |
| 10833 | C * mA * mG * mG * mC * mC | | XXXXX XXXX |
| WV- | mG * m5CeoGeoAeomC * C * C * T * C * T * G * G * A * C * | GCGACCCTCTGGACAGGGAA | XOOOX XXXXX |
| 10834 | A * mG * mG * mG * mA * mA | | XXXXX XXXX |
| WV- | mU * Geom5Ceom5CeomC * A * G * C * A * C * G * C * A * | UGCCCAGCACGCAGGCCAGG | XOOOX XXXXX |
| 10835 | G * G * mC * mC * mA * mG * mG | | XXXXX XXXX |
| WV- | mA * AeoAeoGeomC * C * T * G * G * C * C * T * C * A * G | AAAGCCTGGCCTCAGGGAGG | XOOOX XXXXX |
| 10836 | * mG * mG * mA * mG * mG | | XXXXX XXXX |
| WV- | mG * m5Ceom5Ceom5CeomA * G * C * A * C * G * C * A * | GCCCAGCACGCAGGCCAGGG | XOOOX XXXXX |
| 10837 | G * G * C * mC * mA * mG * mG * mG | | XXXXX XXXX |
| WV- | mC * m5Ceom5CeoAeomG * C * A * C * G * C * A * G * G * | CCCAGCACGCAGGCCAGGGG | XOOOX XXXXX |
| 10838 | C * C * mA * mG * mG * mG * mG | | XXXXX XXXX |
| WV- | mG * Geom5Ceom5CeomA * T * G * C * C * C * A * G * C * | GGCCATGCCCAGCACGCAGG | XOOOX XXXXX |
| 10839 | A * C * mG * mC * mA * mG * mG | | XXXXX XXXX |
| WV- | mC * Geom5CeoAeomG * G * C * C * A * G * G * G * G * C | CGCAGGCCAGGGGCGCGGGG | XOOOX XXXXX |
| 10840 | * G * mC * mG * mG * mG * mG | | XXXXX XXXX |
| WV- | mA * Geom5CeoAeomC * G * C * A * G * G * C * C * A * G | AGCACGCAGGCCAGGGGCGC | XOOOX XXXXX |
| 10841 | * G * mG * mG * mC * mG * mC | | XXXXX XXXX |
| WV- | mC * m5CeoAeoGeomC * A * C * G * C * A * G * G * C * C | CCAGCACGCAGGCCAGGGGC | XOOOX XXXXX |
| 10842 | * A * mG * mG * mG * mG * mC | | XXXXX XXXX |
| WV- | mG * m5CeoAeoGeomG * C * C * A * G * G * G * G * C * G | GCAGGCCAGGGGCGCGGGGC | XOOOX XXXXX |
| 10843 | * C * mG * mG * mG * mG * mC | | XXXXX XXXX |
| WV- | mC * SmAmC * SA * SA * SG * SG * SG * SC * SG * SC * | CACAAGGGCGCAGACU | SOSSS SSSSS RSSOS |
| 10862 | RA * SG * SmAmC * SmU | | |
| WV- | mA * SmGmG * SG * SC * SG * SC * RA * SG * SmAmC * | AGGGCGCAGACU | SOSSS SRSSOS |
| 10863 | SmU | | |
| WV- | mG * SmUmGmCmA * SC * SA * SC * SA * SG * ST * SG * | GUGCACACAGTGGATGAGGG | SOOOS SSSSS SSRSS |
| 10864 | SG * RA * ST * SmGmAmGmG * SmG | | OOOS |
| WV- | mG * SmCmA * SC * SA * SC * SA * SG * ST * SG * SG * | GCACACAGTGGATGAG | SOSSS SSSSS RSSOS |
| 10865 | RA * ST * SmGmA * SmG | | |
| WV- | mA * SC * SA * SG * ST * SG * SG * RA * ST * SmGmA * | ACAGTGGATGAG | SSSSS SRSSOS |
| 10866 | SmG | | |
| WV- | mA * SmCmA * SG * ST * SG * SG * RA * ST * SmGmA * | ACAGTGGATGAG | SOSSS SRSSOS |
| 10867 | SmG | | |
| WV- | mG * mA * mG * mC * mG * T * A * G * A * A * A * C * C * | GAGCGTAGAAACCCTCCAAA | XXXXX XXXXX |
| 10889 | C * T * mC * mc * mA * mA * mA | | XXXXX XXXX |
| WV- | mU * mG * mA * mG * mC * G * T * A * G * A * A * A * C * | UGAGCGTAGAAACCCUCCAA | XXXXX XXXXX |
| 10890 | C * C * mU * mC * mC * mA * mA | | XXXXX XXXX |
| WV- | mC * mU * mG * mA * mG * C * G * T * A * G * A * A * A * | CUGAGCGTAGAAACCCUCCA | XXXXX XXXXX |
| 10891 | C * C * mC * mU * mC * mC * mA | | XXXXX XXXX |
| WV- | mG * mC * mU * mG * mA * G * C * G * T * A * G * A * A * | GCUGAGCGTAGAAACCCUCC | XXXXX XXXXX |
| 10892 | A * C * mC * mC * mU * mC * mC | | XXXXX XXXX |
| WV- | mG * mG * mC * mU * mG * A * G * C * G * T * A * G * A * | GGCUGAGCGTAGAAACCCUC | XXXXX XXXXX |
| 10893 | A * A * mC * mC * mC * mU * mC | | XXXXX XXXX |
| WV- | mA * mG * mG * mc * mU * G * A * G * C * G * T * A * G * | AGGCUGAGCGTAGAAACCCU | XXXXX XXXXX |
| 10894 | A * A * mA * mC * mC * mC * mU | | XXXXX XXXX |
| WV- | mA * mA * mG * mG * mC * T * G * A * G * C * G * T * A * | AAGGCTGAGCGTAGAAACCC | XXXXX XXXXX |
| 10895 | G * A * mA * mA * mC * mC * mC | | XXXXX XXXX |
| WV- | mC * mA * mA * mG * mG * C * T * G * A * G * C * G * T * | CAAGGCTGAGCGTAGAAACC | XXXXX XXXXX |
| 10896 | A * G * mA * mA * mA * mC * mC | | XXXXX XXXX |
| WV- | mC * mC * mA * mA * mG * G * C * T * G * A * G * C * G * | CCAAGGCTGAGCGTAGAAAC | XXXXX XXXXX |
| 10897 | T * A * mG * mA * mA * mA * mC | | XXXXX XXXX |
| WV- | mU * mC * mC * mA * mA * G * G * C * T * G * A * G * C * | UCCAAGGCTGAGCGTAGAAA | XXXXX XXXXX |
| 10898 | G * T * mA * mG * mA * mA * mA | | XXXXX XXXX |
| WV- | mG * mAmGmCmG * T * A * G * A * A * A * C * C * C * T * | GAGCGTAGAAACCCTCCAAA | XOOOX XXXXX |
| 10899 | mCmCmAmA * mA | | XXXXX OOOX |
| WV- | mU * mGmAmGmC * G * T * A * G * A * A * A * C * C * C | UGAGCGTAGAAACCCUCCAA | XOOOX XXXXX |
| 10900 | * mUmCmCmA * mA | | XXXXX OOOX |
| WV- | mC * mUmGmAmG * C * G * T * A * G * A * A * A * C * C | CUGAGCGTAGAAACCCUCCA | XOOOX XXXXX |
| 10901 | * mCmUmCmC * mA | | XXXXX OOOX |
| WV- | mG * mCmUmGmA * G * C * G * T * A * G * A * A * A * C | GCUGAGCGTAGAAACCCUCC | XOOOX XXXXX |
| 10902 | * mCmCmUmC * mc | | XXXXX OOOX |
| WV- | mG * mGmCmUmG * A * G * C * G * T * A * G * A * A * A | GGCUGAGCGTAGAAACCCUC | XOOOX XXXXX |
| 10903 | * mCmCmCmU * mc | | XXXXX OOOX |
| WV- | mA * mGmGmCmU * G * A * G * C * G * T * A * G * A * A | AGGCUGAGCGTAGAAACCCU | XOOOX XXXXX |
| 10904 | * mAmCmCmC * mU | | XXXXX OOOX |
| WV- | mA * mAmGmGmC * T * G * A * G * C * G * T * A * G * A | AAGGCTGAGCGTAGAAACCC | XOOOX XXXXX |
| 10905 | * mAmAmCmC * mC | | XXXXX OOOX |
| WV- | mC * mAmAmGmG * C * T * G * A * G * C * G * T * A * G * | CAAGGCTGAGCGTAGAAACC | XOOOX XXXXX |
| 10906 | mAmAmAmC * mC | | XXXXX OOOX |
| WV- | mC * mCmAmAmG * G * C * T * G * A * G * C * G * T * A * | CCAAGGCTGAGCGTAGAAAC | XOOOX XXXXX |
| 10907 | mGmAmAmA * mC | | XXXXX OOOX |
| WV- | mU * mCmCmAmA * G * G * C * T * G * A * G * C * G * T * | UCCAAGGCTGAGCGTAGAAA | XOOOX XXXXX |
| 10908 | mAmGmAmA * mA | | XXXXX OOOX |
| WV- | Geo * Aeo * Geo * m5Ceo * Geo * T * A * G * A * A * A * C | GAGCGTAGAAACCCTCCAAA | XXXXX XXXXX |
| 10909 | * C * C * T * m5Ceo * m5Ceo * Aeo * Aeo * Aeo | | XXXXX XXXX |
| WV- | Teo * Geo * Aeo * Geo * m5Ceo * G * T * A * G * A * A * A * | TGAGCGTAGAAACCCTCCAA | XXXXX XXXXX |
| 10910 | C * C * C * Teo * m5Ceo * m5Ceo * Aeo * Aeo | | XXXXX XXXX |
| WV- | m5Ceo * Teo * Geo * Aeo * Geo * C * G * T * A * G * A * A * | CTGAGCGTAGAAACCCTCCA | XXXXX XXXXX |
| 10911 | A * C * C * m5Ceo * Teo * m5Ceo * m5Ceo * Aeo | | XXXXX XXXX |
| WV- | Geo * m5Ceo * Teo * Geo * Aeo * G * C * G * T * A * G * A * | GCTGAGCGTAGAAACCCTCC | XXXXX XXXXX |
| 10912 | A * A * C * m5Ceo * m5Ceo * Teo * m5Ceo * m5Ceo | | XXXXX XXXX |
| WV- | Geo * Geo * m5Ceo * Teo * Geo * A * G * C * G * T * A * G * | GGCTGAGCGTAGAAACCCTC | XXXXX XXXXX |
| 10913 | A * A * A * m5Ceo * m5Ceo * m5Ceo * Teo * m5Ceo | | XXXXX XXXX |
| WV- | Aeo * Geo * Geo * m5Ceo * Teo * G * A * G * C * G * T * A * | AGGCTGAGCGTAGAAACCCT | XXXXX XXXXX |
| 10914 | G * A * A * Aeo * m5Ceo * m5Ceo * m5Ceo * Teo | | XXXXX XXXX |
| WV- | Aeo * Aeo * Geo * Geo * m5Ceo * T * G * A * G * C * G * T * | AAGGCTGAGCGTAGAAACCC | XXXXX XXXXX |
| 10915 | A * G * A * Aeo * Aeo * m5Ceo * m5Ceo * m5Ceo | | XXXXX XXXX |
| WV- | m5Ceo * Aeo * Aeo * Geo * Geo * C * T * G * A * G * C * G * | CAAGGCTGAGCGTAGAAACC | XXXXX XXXXX |
| 10916 | T * A * G * Aeo * Aeo * Aeo * m5Ceo * m5Ceo | | XXXXX XXXX |
| WV- | m5Ceo * m5Ceo * Aeo * Aeo * Geo * G * C * T * G * A * G * | CCAAGGCTGAGCGTAGAAAC | XXXXX XXXXX |
| 10917 | C * G * T * A * Geo * Aeo * Aeo * Aeo * m5Ceo | | XXXXX XXXX |
| WV- | Teo * m5Ceo * m5Ceo * Aeo * Aeo * G * G * C * T * G * A * | TCCAAGGCTGAGCGTAGAAA | XXXXX XXXXX |
| 10918 | G * C * G * T * Aeo * Geo * Aeo * Aeo * Aeo | | XXXXX XXXX |
| WV- | Geo * AeoGeom5CeoGeo * T * A * G * A * A * A * C * C * C | GAGCGTAGAAACCCTCCAAA | XOOOX XXXXX |
| 10919 | * T * m5Ceom5CeoAeoAeo * Aeo | | XXXXX OOOX |
| WV- | Teo * GeoAeoGeom5Ceo * G * T * A * G * A * A * A * C * C | TGAGCGTAGAAACCCTCCAA | XOOOX XXXXX |
| 10920 | * C * Teom5Ceom5CeoAeo * Aeo | | XXXXX OOOX |
| WV- | m5Ceo * TeoGeoAeoGeo * C * G * T * A * G * A * A * A * C | CTGAGCGTAGAAACCCTCCA | XOOOX XXXXX |
| 10921 | * C * m5CeoTeom5Ceom5Ceo * Aeo | | XXXXX OOOX |
| WV- | Geo * m5CeoTeoGeoAeo * G * C * G * T * A * G * A * A * A | GCTGAGCGTAGAAACCCTCC | XOOOX XXXXX |
| 10922 | * C * m5Ceom5CeoTeom5Ceo * m5Ceo | | XXXXX OOOX |
| WV- | Geo * Geom5CeoTeoGeo * A * G * C * G * T * A * G * A * A | GGCTGAGCGTAGAAACCCTC | XOOOX XXXXX |
| 10923 | * A * m5Ceom5Ceom5CeoTeo * m5Ceo | | XXXXX OOOX |
| WV- | Aeo * GeoGeom5CeoTeo * G * A * G * C * G * T * A * G * A | AGGCTGAGCGTAGAAACCCT | XOOOX XXXXX |
| 10924 | * A * Aeom5Ceom5Ceom5Ceo * Teo | | XXXXX OOOX |
| WV- | Aeo * AeoGeoGeom5Ceo * T * G * A * G * C * G * T * A * G | AAGGCTGAGCGTAGAAACCC | XOOOX XXXXX |
| 10925 | * A * AeoAeom5Ceom5Ceo * m5Ceo | | XXXXX OOOX |
| WV- | m5Ceo * AeoAeoGeoGeo * C * T * G * A * G * C * G * T * A | CAAGGCTGAGCGTAGAAACC | XOOOX XXXXX |
| 10926 | * G * AeoAeoAeom5Ceo * m5Ceo | | XXXXX OOOX |
| WV- | m5Ceo * m5CeoAeoAeoGeo * G * C * T * G * A * G * C * G * | CCAAGGCTGAGCGTAGAAAC | XOOOX XXXXX |
| 10927 | T * A * GeoAeoAeoAeo * m5Ceo | | XXXXX OOOX |
| WV- | Teo * m5Ceom5CeoAeoAeo * G * G * C * T * G * A * G * C * | TCCAAGGCTGAGCGTAGAAA | XOOOX XXXXX |
| 10928 | G * T * AeoGeoAeoAeo * Aeo | | XXXXX OOOX |
| WV- | mG * AeoGeom5CeomG * T * A * G * A * A * A * C * C * C | GAGCGTAGAAACCCTCCAAA | XOOOX XXXXX |
| 10929 | * T * mC * mC * mA * mA * mA | | XXXXX XXXX |
| WV- | mU * GeoAeoGeomC * G * T * A * G * A * A * A * C * C * C | UGAGCGTAGAAACCCUCCAA | XOOOX XXXXX |
| 10930 | * mU * mC * mC * mA * mA | | XXXXX XXXX |
| WV- | mC * TeoGeoAeomG * C * G * T * A * G * A * A * A * C * C | CTGAGCGTAGAAACCCUCCA | XOOOX XXXXX |
| 10931 | * mC * mU * mC * mC * mA | | XXXXX XXXX |
| WV- | mG * m5CeoTeoGeomA * G * C * G * T * A * G * A * A * A | GCTGAGCGTAGAAACCCUCC | XOOOX XXXXX |
| 10932 | * C * mC * mC * mU * mC * mC | | XXXXX XXXX |
| WV- | mG * Geom5CeoTeomG * A * G * C * G * T * A * G * A * A | GGCTGAGCGTAGAAACCCUC | XOOOX XXXXX |
| 10933 | * A * mC * mC * mC * mU * mC | | XXXXX XXXX |
| WV- | mA * GeoGeom5CeomU * G * A * G * C * G * T * A * G * A | AGGCUGAGCGTAGAAACCCU | XOOOX XXXXX |
| 10934 | * A * mA * mC * mC * mC * mU | | XXXXX XXXX |
| WV- | mA * AeoGeoGeomC * T * G * A * G * C * G * T * A * G * A | AAGGCTGAGCGTAGAAACCC | XOOOX XXXXX |
| 10935 | * mA * mA * mC * mC * mC | | XXXXX XXXX |
| WV- | mC * AeoAeoGeomG * C * T * G * A * G * C * G * T * A * G | CAAGGCTGAGCGTAGAAACC | XOOOX XXXXX |
| 10936 | * mA * mA * mA * mC * mC | | XXXXX XXXX |
| WV- | mC * m5CeoAeoAeomG * G * C * T * G * A * G * C * G * T * | CCAAGGCTGAGCGTAGAAAC | XOOOX XXXXX |
| 10937 | A * mG * mA * mA * mA * mC | | XXXXX XXXX |
| WV- | mU * m5Ceom5CeoAeomA * G * G * C * T * G * A * G * C * | UCCAAGGCTGAGCGTAGAAA | XOOOX XXXXX |
| 10938 | G * T * mA * mG * mA * mA * mA | | XXXXX XXXX |
| WV- | mA * mU * mC * mU * mG * A * A * G * C * A * G * C * A * | AUCUGAAGCAGCAGCUUCUC | XXXXX XXXXX |
| 10939 | G * C * mU * mU * mC * mU * mC | | XXXXX XXXX |
| WV- | mG * mA * mU * mC * mU * G * A * A * G * C * A * G * C * | GAUCUGAAGCAGCAGCUUCU | XXXXX XXXXX |
| 10940 | A * G * mC * mU * mU * mC * mU | | XXXXX XXXX |
| WV- | mU * mG * mA * mU * mC * T * G * A * A * G * C * A * G * | UGAUCTGAAGCAGCAGCUUC | XXXXX XXXXX |
| 10941 | C * A * mG * mC * mU * mU * mC | | XXXXX XXXX |
| WV- | mU * mU * mG * mA * mU * C * T * G * A * A * G * C * A * | UUGAUCTGAAGCAGCAGCUU | XXXXX XXXXX |
| 10942 | G * C * mA * mG * mC * mU * mU | | XXXXX XXXX |
| WV- | mG * mU * mU * mG * mA * T * C * T * G * A * A * G * C * | GUUGATCTGAAGCAGCAGCU | XXXXX XXXXX |
| 10943 | A * G * mC * mA * mG * mC * mU | | XXXXX XXXX |
| WV- | mG * mG * mU * mU * mG * A * T * C * T * G * A * A * G * | GGUUGATCTGAAGCAGCAGC | XXXXX XXXXX |
| 10944 | C * A * mG * mC * mA * mG * mC | | XXXXX XXXX |
| WV- | mG * mG * mG * mU * mU * G * A * T * C * T * G * A * A * | GGGUUGATCTGAAGCAGCAG | XXXXX XXXXX |
| 10945 | G * C * mA * mG * mC * mA * mG | | XXXXX XXXX |
| WV- | mG * mG * mG * mG * mU * T * G * A * T * C * T * G * A * | GGGGUTGATCTGAAGCAGCA | XXXXX XXXXX |
| 10946 | A * G * mC * mA * mG * mC * mA | | XXXXX XXXX |
| WV- | mC * mG * mG * mG * mG * T * T * G * A * T * C * T * G * | CGGGGTTGATCTGAAGCAGC | XXXXX XXXXX |
| 10947 | A * A * mG * mC * mA * mG * mC | | XXXXX XXXX |
| WV- | mU * mC * mG * mG * mG * G * T * T * G * A * T * C * T * | UCGGGGTTGATCTGAAGCAG | XXXXX XXXXX |
| 10948 | G * A * mA * mG * mC * mA * mG | | XXXXX XXXX |
| WV- | mA * mUmCmUmG * A * A * G * C * A * G * C * A * G * C | AUCUGAAGCAGCAGCUUCUC | XOOOX XXXXX |
| 10949 | * mUmUmCmU * mC | | XXXXX OOOX |
| WV- | mG * mAmUmCmU * G * A * A * G * C * A * G * C * A * G | GAUCUGAAGCAGCAGCUUCU | XOOOX XXXXX |
| 10950 | * mCmUmUmC * mU | | XXXXX OOOX |
| WV- | mU * mGmAmUmC * T * G * A * A * G * C * A * G * C * A | UGAUCTGAAGCAGCAGCUUC | XOOOX XXXXX |
| 10951 | * mGmCmUmU * mC | | XXXXX OOOX |
| WV- | mU * mUmGmAmU * C * T * G * A * A * G * C * A * G * C | UUGAUCTGAAGCAGCAGCUU | XOOOX XXXXX |
| 10952 | * mAmGmCmU * mU | | XXXXX OOOX |
| WV- | mG * mUmUmGmA * T * C * T * G * A * A * G * C * A * G | GUUGATCTGAAGCAGCAGCU | XOOOX XXXXX |
| 10953 | * mCmAmGmC * mU | | XXXXX OOOX |
| WV- | mG * mGmUmUmG * A * T * C * T * G * A * A * G * C * A | GGUUGATCTGAAGCAGCAGC | XOOOX XXXXX |
| 10954 | * mGmCmAmG * mC | | XXXXX OOOX |
| WV- | mG * mGmGmUmU * G * A * T * C * T * G * A * A * G * C | GGGUUGATCTGAAGCAGCAG | XOOOX XXXXX |
| 10955 | * mAmGmCmA * mG | | XXXXX OOOX |
| WV- | mG * mGmGmGmU * T * G * A * T * C * T * G * A * A * G * | GGGGUTGATCTGAAGCAGCA | XOOOX XXXXX |
| 10956 | mCmAmGmC * mA | | XXXXX OOOX |
| WV- | mC * mGmGmGmG * T * T * G * A * T * C * T * G * A * A * | CGGGGTTGATCTGAAGCAGC | XOOOX XXXXX |
| 10957 | mGmCmAmG * mC | | XXXXX OOOX |
| WV- | mU * mCmGmGmG * G * T * T * G * A * T * C * T * G * A * | UCGGGGTTGATCTGAAGCAG | XOOOX XXXXX |
| 10958 | mAmGmCmA * mG | | XXXXX OOOX |
| WV- | Aeo * Teo * m5Ceo * Teo * Geo * A * A * G * C * A * G * C * | ATCTGAAGCAGCAGCTTCTC | XXXXX XXXXX |
| 10959 | A * G * C * Teo * Teo * m5Ceo * Teo * m5Ceo | | XXXXX XXXX |
| WV- | Geo * Aeo * Teo * m5Ceo * Teo * G * A * A * G * C * A * G * | GATCTGAAGCAGCAGCTTCT | XXXXX XXXXX |
| 10960 | C * A * G * m5Ceo * Teo * Teo * m5Ceo * Teo | | XXXXX XXXX |
| WV- | Teo * Geo * Aeo * Teo * m5Ceo * T * G * A * A * G * C * A * | TGATCTGAAGCAGCAGCTTC | XXXXX XXXXX |
| 10961 | G * C * A * Geo * m5Ceo * Teo * Teo * m5Ceo | | XXXXX XXXX |
| WV- | Teo * Teo * Geo * Aeo * Teo * C * T * G * A * A * G * C * A | TTGATCTGAAGCAGCAGCTT | XXXXX XXXXX |
| 10962 | * G * C * Aeo * Geo * m5Ceo * Teo * Teo | | XXXXX XXXX |
| WV- | Geo * Teo * Teo * Geo * Aeo * T * C * T * G * A * A * G * C | GTTGATCTGAAGCAGCAGCT | XXXXX XXXXX |
| 10963 | * A * G * m5Ceo * Aeo * Geo * m5Ceo * Teo | | XXXXX XXXX |
| WV- | Geo * Geo * Teo * Teo * Geo * A * T * C * T * G * A * A * G | GGTTGATCTGAAGCAGCAGC | XXXXX XXXXX |
| 10964 | * C * A * Geo * m5Ceo * Aeo * Geo * m5Ceo | | XXXXX XXXX |
| WV- | Geo * Geo * Geo * Teo * Teo * G * A * T * C * T * G * A * A | GGGTTGATCTGAAGCAGCAG | XXXXX XXXXX |
| 10965 | * G * C * Aeo * Geo * m5Ceo * Aeo * Geo | | XXXXX XXXX |
| WV- | Geo * Geo * Geo * Geo * Teo * T * G * A * T * C * T * G * A | GGGGTTGATCTGAAGCAGCA | XXXXX XXXXX |
| 10966 | * A * G * m5Ceo * Aeo * Geo * m5Ceo * Aeo | | XXXXX XXXX |
| WV- | m5Ceo * Geo * Geo * Geo * Geo * T * T * G * A * T * C * T * | CGGGGTTGATCTGAAGCAGC | XXXXX XXXXX |
| 10967 | G * A * A * Geo * m5Ceo * Aeo * Geo * m5Ceo | | XXXXX XXXX |
| WV- | Teo * m5Ceo * Geo * Geo * Geo * G * T * T * G * A * T * C * | TCGGGGTTGATCTGAAGCAG | XXXXX XXXXX |
| 10968 | T * G * A * Aeo * Geo * m5Ceo * Aeo * Geo | | XXXXX XXXX |
| WV- | Aeo * Teom5CeoTeoGeo * A * A * G * C * A * G * C * A * G | ATCTGAAGCAGCAGCTTCTC | XOOOX XXXXX |
| 10969 | * C * TeoTeom5CeoTeo * m5Ceo | | XXXXX OOOX |
| WV- | Geo * AeoTeom5CeoTeo * G * A * A * G * C * A * G * C * A | GATCTGAAGCAGCAGCTTCT | XOOOX XXXXX |
| 10970 | * G * m5CeoTeoTeom5Ceo * Teo | | XXXXX OOOX |
| WV- | Teo * GeoAeoTeom5Ceo * T * G * A * A * G * C * A * G * C | TGATCTGAAGCAGCAGCTTC | XOOOX XXXXX |
| 10971 | * A * Geom5CeoTeoTeo * m5Ceo | | XXXXX OOOX |
| WV- | Teo * TeoGeoAeoTeo * C * T * G * A * A * G * C * A * G * C | TTGATCTGAAGCAGCAGCTT | XOOOX XXXXX |
| 10972 | * AeoGeom5CeoTeo * Teo | | XXXXX OOOX |
| WV- | Geo * TeoTeoGeoAeo * T * C * T * G * A * A * G * C * A * G | GTTGATCTGAAGCAGCAGCT | XOOOX XXXXX |
| 10973 | * m5CeoAeoGeom5Ceo * Teo | | XXXXX OOOX |
| WV- | Geo * GeoTeoTeoGeo * A * T * C * T * G * A * A * G * C * A | GGTTGATCTGAAGCAGCAGC | XOOOX XXXXX |
| 10974 | * Geom5CeoAeoGeo * m5Ceo | | XXXXX OOOX |
| WV- | Geo * GeoGeoTeoTeo * G * A * T * C * T * G * A * A * G * C | GGGTTGATCTGAAGCAGCAG | XOOOX XXXXX |
| 10975 | * AeoGeom5CeoAeo * Geo | | XXXXX OOOX |
| WV- | Geo * GeoGeoGeoTeo * T * G * A * T * C * T * G * A * A * G | GGGGTTGATCTGAAGCAGCA | XOOOX XXXXX |
| 10976 | * m5CeoAeoGeom5Ceo * Aeo | | XXXXX OOOX |
| WV- | m5Ceo * GeoGeoGeoGeo * T * T * G * A * T * C * T * G * A | CGGGGTTGATCTGAAGCAGC | XOOOX XXXXX |
| 10977 | * A * Geom5CeoAeoGeo * m5Ceo | | XXXXX OOOX |
| WV- | Teo * m5CeoGeoGeoGeo * G * T * T * G * A * T * C * T * G | TCGGGGTTGATCTGAAGCAG | XOOOX XXXXX |
| 10978 | * A * AeoGeom5CeoAeo * Geo | | XXXXX OOOX |
| WV- | mA * Teom5CeoTeomG * A * A * G * C * A * G * C * A * G | ATCTGAAGCAGCAGCUUCUC | XOOOX XXXXX |
| 10979 | * C * mU * mU * mC * mU * mC | | XXXXX XXXX |
| WV- | mG * AeoTeom5CeomU * G * A * A * G * C * A * G * C * A | GATCUGAAGCAGCAGCUUCU | XOOOX XXXXX |
| 10980 | * G * mC * mU * mU * mC * mU | | XXXXX XXXX |
| WV- | mU * GeoAeoTeomC * T * G * A * A * G * C * A * G * C * A | UGATCTGAAGCAGCAGCUUC | XOOOX XXXXX |
| 10981 | * mG * mC * mU * mU * mC | | XXXXX XXXX |
| WV- | mU * TeoGeoAeomU * C * T * G * A * A * G * C * A * G * C | UTGAUCTGAAGCAGCAGCUU | XOOOX XXXXX |
| 10982 | * mA * mG * mC * mU * mU | | XXXXX XXXX |
| WV- | mG * TeoTeoGeomA * T * C * T * G * A * A * G * C * A * G | GTTGATCTGAAGCAGCAGCU | XOOOX XXXXX |
| 10983 | * mC * mA * mG * mC * mU | | XXXXX XXXX |
| WV- | mG * GeoTeoTeomG * A * T * C * T * G * A * A * G * C * A | GGTTGATCTGAAGCAGCAGC | XOOOX XXXXX |
| 10984 | * mG * mC * mA * mG * mC | | XXXXX XXXX |
| WV- | mG * GeoGeoTeomU * G * A * T * C * T * G * A * A * G * C | GGGTUGATCTGAAGCAGCAG | XOOOX XXXXX |
| 10985 | * mA * mG * mC * mA * mG | | XXXXX XXXX |
| WV- | mG * GeoGeoGeomU * T * G * A * T * C * T * G * A * A * G | GGGGUTGATCTGAAGCAGCA | XOOOX XXXXX |
| 10986 | * mC * mA * mG * mC * mA | | XXXXX XXXX |
| WV- | mC * GeoGeoGeomG * T * T * G * A * T * C * T * G * A * A | CGGGGTTGATCTGAAGCAGC | XOOOX XXXXX |
| 10987 | * mG * mC * mA * mG * mC | | XXXXX XXXX |
| WV- | mU * m5CeoGeoGeomG * G * T * T * G * A * T * C * T * G * | UCGGGGTTGATCTGAAGCAG | XOOOX XXXXX |
| 10988 | A * mA * mG * mC * mA * mG | | XXXXX XXXX |
| WV- | mG * mA * mG * mG * mA * G * G * C * C * G * T * G * C * | GAGGAGGCCGTGCAGGGCUC | XXXXX XXXXX |
| 10989 | A * G * mG * mG * mC * mU * mC | | XXXXX XXXX |
| WV- | mA * mG * mA * mG * mG * A * G * G * C * C * G * T * G * | AGAGGAGGCCGTGCAGGGCU | XXXXX XXXXX |
| 10990 | C * A * mG * mG * mG * mC * mU | | XXXXX XXXX |
| WV- | mU * mA * mG * mA * mG * G * A * G * G * C * C * G * T * | UAGAGGAGGCCGTGCAGGGC | XXXXX XXXXX |
| 10991 | G * C * mA * mG * mG * mG * mC | | XXXXX XXXX |
| WV- | mA * mU * mA * mG * mA * G * G * A * G * G * C * C * G * | AUAGAGGAGGCCGTGCAGGG | XXXXX XXXXX |
| 10992 | T * G * mC * mA * mG * mG * mG | | XXXXX XXXX |
| WV- | mC * mA * mU * mA * mG * A * G * G * A * G * G * C * C * | CAUAGAGGAGGCCGTGCAGG | XXXXX XXXXX |
| 10993 | G * T * mG * mC * mA * mG * mG | | XXXXX XXXX |
| WV- | mA * mC * mA * mU * mA * G * A * G * G * A * G * G * C * | ACAUAGAGGAGGCCGUGCAG | XXXXX XXXXX |
| 10994 | C * G * mU * mG * mC * mA * mG | | XXXXX XXXX |
| WV- | mC * mA * mC * mA * mU * A * G * A * G * G * A * G * G * | CACAUAGAGGAGGCCGUGCA | XXXXX XXXXX |
| 10995 | C * C * mG * mU * mG * mC * mA | | XXXXX XXXX |
| WV- | mG * mC * mA * mC * mA * T * A * G * A * G * G * A * G * | GCACATAGAGGAGGCCGUGC | XXXXX XXXXX |
| 10996 | G * C * mC * mG * mU * mG * mC | | XXXXX XXXX |
| WV- | mA * mG * mC * mA * mC * A * T * A * G * A * G * G * A * | AGCACATAGAGGAGGCCGUG | XXXXX XXXXX |
| 10997 | G * G * mC * mC * mG * mU * mG | | XXXXX XXXX |
| WV- | mC * mA * mG * mC * mA * C * A * T * A * G * A * G * G * | CAGCACATAGAGGAGGCCGU | XXXXX XXXXX |
| 10998 | A * G * mG * mC * mC * mG * mU | | XXXXX XXXX |
| WV- | mG * mAmGmGmA * G * G * C * C * G * T * G * C * A * G | GAGGAGGCCGTGCAGGGCUC | XOOOX XXXXX |
| 10999 | * mGmGmCmU * mC | | XXXXX OOOX |
| WV- | mA * mGmAmGmG * A * G * G * C * C * G * T * G * C * A | AGAGGAGGCCGTGCAGGGCU | XOOOX XXXXX |
| 11000 | * mGmGmGmC * mU | | XXXXX OOOX |
| WV- | mU * mAmGmAmG * G * A * G * G * C * C * G * T * G * C | UAGAGGAGGCCGTGCAGGGC | XOOOX XXXXX |
| 11001 | * mAmGmGmG * mC | | XXXXX OOOX |
| WV- | mA * mUmAmGmA * G * G * A * G * G * C * C * G * T * G | AUAGAGGAGGCCGTGCAGGG | XOOOX XXXXX |
| 11002 | * mCmAmGmG * mG | | XXXXX OOOX |
| WV- | mC * mAmUmAmG * A * G * G * A * G * G * C * C * G * T | CAUAGAGGAGGCCGTGCAGG | XOOOX XXXXX |
| 11003 | * mGmCmAmG * mG | | XXXXX OOOX |
| WV- | mA * mCmAmUmA * G * A * G * G * A * G * G * C * C * G | ACAUAGAGGAGGCCGUGCAG | XOOOX XXXXX |
| 11004 | * mUmGmCmA * mG | | XXXXX OOOX |
| WV- | mC * mAmCmAmU * A * G * A * G * G * A * G * G * C * C | CACAUAGAGGAGGCCGUGCA | XOOOX XXXXX |
| 11005 | * mGmUmGmC * mA | | XXXXX OOOX |
| WV- | mG * mCmAmCmA * T * A * G * A * G * G * A * G * G * C | GCACATAGAGGAGGCCGUGC | XOOOX XXXXX |
| 11006 | * mCmGmUmG * mC | | XXXXX OOOX |
| WV- | mA * mGmCmAmC * A * T * A * G * A * G * G * A * G * G | AGCACATAGAGGAGGCCGUG | XOOOX XXXXX |
| 11007 | * mCmCmGmU * mG | | XXXXX OOOX |
| WV- | mC * mAmGmCmA * C * A * T * A * G * A * G * G * A * G | CAGCACATAGAGGAGGCCGU | XOOOX XXXXX |
| 11008 | * mGmCmCmG * mU | | XXXXX OOOX |
| WV- | Geo * Aeo * Geo * Geo * Aeo * G * G * C * C * G * T * G * C | GAGGAGGCCGTGCAGGGCTC | XXXXX XXXXX |
| 11009 | * A * G * Geo * Geo * m5Ceo * Teo * m5Ceo | | XXXXX XXXX |
| WV- | Aeo * Geo * Aeo * Geo * Geo * A * G * G * C * C * G * T * G | AGAGGAGGCCGTGCAGGGCT | XXXXX XXXXX |
| 11010 | * C * A * Geo * Geo * Geo * m5Ceo * Teo | | XXXXX XXXX |
| WV- | Teo * Aeo * Geo * Aeo * Geo * G * A * G * G * C * C * G * T | TAGAGGAGGCCGTGCAGGGC | XXXXX XXXXX |
| 11011 | * G * C * Aeo * Geo * Geo * Geo * m5Ceo | | XXXXX XXXX |
| WV- | Aeo * Teo * Aeo * Geo * Aeo * G * G * A * G * G * C * C * G | ATAGAGGAGGCCGTGCAGGG | XXXXX XXXXX |
| 11012 | * T * G * m5Ceo * Aeo * Geo * Geo * Geo | | XXXXX XXXX |
| WV- | m5Ceo * Aeo * Teo * Aeo * Geo * A * G * G * A * G * G * C | CATAGAGGAGGCCGTGCAGG | XXXXX XXXXX |
| 11013 | * C * G * T * Geo * m5Ceo * Aeo * Geo * Geo | | XXXXX XXXX |
| WV- | Aeo * m5Ceo * Aeo * Teo * Aeo * G * A * G * G * A * G * G | ACATAGAGGAGGCCGTGCAG | XXXXX XXXXX |
| 11014 | * C * C * G * Teo * Geo * m5Ceo * Aeo * Geo | | XXXXX XXXX |
| WV- | m5Ceo * Aeo * m5Ceo * Aeo * Teo * A * G * A * G * G * A * | CACATAGAGGAGGCCGTGCA | XXXXX XXXXX |
| 11015 | G * G * C * C * Geo * Teo * Geo * m5Ceo * Aeo | | XXXXX XXXX |
| WV- | Geo * m5Ceo * Aeo * m5Ceo * Aeo * T * A * G * A * G * G * | GCACATAGAGGAGGCCGTGC | XXXXX XXXXX |
| 11016 | A * G * G * C * m5Ceo * Geo * Teo * Geo * m5Ceo | | XXXXX XXXX |
| WV- | Aeo * Geo * m5Ceo * Aeo * m5Ceo * A * T * A * G * A * G * | AGCACATAGAGGAGGCCGTG | XXXXX XXXXX |
| 11017 | G * A * G * G * m5Ceo * m5Ceo * Geo * Teo * Geo | | XXXXX XXXX |
| WV- | m5Ceo * Aeo * Geo * m5Ceo * Aeo * C * A * T * A * G * A * | CAGCACATAGAGGAGGCCGT | XXXXX XXXXX |
| 11018 | G * G * A * G * Geo * m5Ceo * m5Ceo * Geo * Teo | | XXXXX XXXX |
| WV- | Geo * AeoGeoGeoAeo * G * G * C * C * G * T * G * C * A * | GAGGAGGCCGTGCAGGGCTC | XOOOX XXXXX |
| 11019 | G * GeoGeom5CeoTeo * m5Ceo | | XXXXX OOOX |
| WV- | Aeo * GeoAeoGeoGeo * A * G * G * C * C * G * T * G * C * | AGAGGAGGCCGTGCAGGGCT | XOOOX XXXXX |
| 11020 | A * GeoGeoGeom5Ceo * Teo | | XXXXX OOOX |
| WV- | Teo * AeoGeoAeoGeo * G * A * G * G * C * C * G * T * G * | TAGAGGAGGCCGTGCAGGGC | XOOOX XXXXX |
| 11021 | C * AeoGeoGeoGeo * m5Ceo | | XXXXX OOOX |
| WV- | Aeo * TeoAeoGeoAeo * G * G * A * G * G * C * C * G * T * | ATAGAGGAGGCCGTGCAGGG | XOOOX XXXXX |
| 11022 | G * m5CeoAeoGeoGeo * Geo | | XXXXX OOOX |
| WV- | m5Ceo * AeoTeoAeoGeo * A * G * G * A * G * G * C * C * G | CATAGAGGAGGCCGTGCAGG | XOOOX XXXXX |
| 11023 | * T * Geom5CeoAeoGeo * Geo | | XXXXX OOOX |
| WV- | Aeo * m5CeoAeoTeoAeo * G * A * G * G * A * G * G * C * C | ACATAGAGGAGGCCGTGCAG | XOOOX XXXXX |
| 11024 | * G * TeoGeom5CeoAeo * Geo | | XXXXX OOOX |
| WV- | m5Ceo * Aeom5CeoAeoTeo * A * G * A * G * G * A * G * G | CACATAGAGGAGGCCGTGCA | XOOOX XXXXX |
| 11025 | * C * C * GeoTeoGeom5Ceo * Aeo | | XXXXX OOOX |
| WV- | Geo * m5CeoAeom5CeoAeo * T * A * G * A * G * G * A * G | GCACATAGAGGAGGCCGTGC | XOOOX XXXXX |
| 11026 | * G * C * m5CeoGeoTeoGeo * m5Ceo | | XXXXX OOOX |
| WV- | Aeo * Geom5CeoAeom5Ceo * A * T * A * G * A * G * G * A | AGCACATAGAGGAGGCCGTG | XOOOX XXXXX |
| 11027 | * G * G * m5Ceom5CeoGeoTeo * Geo | | XXXXX OOOX |
| WV- | m5Ceo * AeoGeom5CeoAeo * C * A * T * A * G * A * G * G * | CAGCACATAGAGGAGGCCGT | XOOOX XXXXX |
| 11028 | A * G * Geom5Ceom5CeoGeo * Teo | | XXXXX OOOX |
| WV- | mG * AeoGeoGeomA * G * G * C * C * G * T * G * C * A * G | GAGGAGGCCGTGCAGGGCUC | XOOOX XXXXX |
| 11029 | * mG * mG * mC * mU * mC | | XXXXX XXXX |
| WV- | mA * GeoAeoGeomG * A * G * G * C * C * G * T * G * C * A | AGAGGAGGCCGTGCAGGGCU | XOOOX XXXXX |
| 11030 | * mG * mG * mG * mC * mU | | XXXXX XXXX |
| WV- | mU * AeoGeoAeomG * G * A * G * G * C * C * G * T * G * C | UAGAGGAGGCCGTGCAGGGC | XOOOX XXXXX |
| 11031 | * mA * mG * mG * mG * mC | | XXXXX XXXX |
| WV- | mA * TeoAeoGeomA * G * G * A * G * G * C * C * G * T * G | ATAGAGGAGGCCGTGCAGGG | XOOOX XXXXX |
| 11032 | * mC * mA * mG * mG * mG | | XXXXX XXXX |
| WV- | mC * AeoTeoAeomG * A * G * G * A * G * G * C * C * G * T | CATAGAGGAGGCCGTGCAGG | XOOOX XXXXX |
| 11033 | * mG * mC * mA * mG * mG | | XXXXX XXXX |
| WV- | mA * m5CeoAeoTeomA * G * A * G * G * A * G * G * C * C | ACATAGAGGAGGCCGUGCAG | XOOOX XXXXX |
| 11034 | * G * mU * mG * mC * mA * mG | | XXXXX XXXX |
| WV- | mC * Aeom5CeoAeomU * A * G * A * G * G * A * G * G * C | CACAUAGAGGAGGCCGUGCA | XOOOX XXXXX |
| 11035 | * C * mG * mU * mG * mC * mA | | XXXXX XXXX |
| WV- | mG * m5CeoAeom5CeomA * T * A * G * A * G * G * A * G * | GCACATAGAGGAGGCCGUGC | XOOOX XXXXX |
| 11036 | G * C * mC * mG * mU * mG * mC | | XXXXX XXXX |
| WV- | mA * Geom5CeoAeomC * A * T * A * G * A * G * G * A * G | AGCACATAGAGGAGGCCGUG | XOOOX XXXXX |
| 11037 | * G * mC * mC * mG * mU * mG | | XXXXX XXXX |
| WV- | mC * AeoGeom5CeomA * C * A * T * A * G * A * G * G * A | CAGCACATAGAGGAGGCCGU | XOOOX XXXXX |
| 11038 | * G * mG * mC * mC * mG * mU | | XXXXX XXXX |
| WV- | mA * m5CeoAeoGeomU * A * G * A * T * G * A * G * G * G | ACAGUAGATGAGGGAGGAGG | XOOOX XXXXX |
| 11115 | * A * mG * mG * mA * mG * mG | | XXXXX XXXX |
| WV- | mC * Aeom5CeoAeomG * T * A * G * A * T * G * A * G * G | CACAGTAGATGAGGGAGGAG | XOOOX XXXXX |
| 11116 | * G * mA * mG * mG * mA * mG | | XXXXX XXXX |
| WV- | mA * m5CeoAeom5CeomA * G * T * A * G * A * T * G * A * | ACACAGTAGATGAGGGAGGA | XOOOX XXXXX |
| 11117 | G * G * mG * mA * mG * mG * mA | | XXXXX XXXX |
| WV- | mC * Aeom5CeoAeomC * A * G * T * A * G * A * T * G * A | CACACAGTAGATGAGGGAGG | XOOOX XXXXX |
| 11118 | * G * mG * mG * mA * mG * mG | | XXXXX XXXX |
| WV- | mG * m5CeoAeom5CeomA * C * A * G * T * A * G * A * T * | GCACACAGTAGATGAGGGAG | XOOOX XXXXX |
| 11119 | G * A * mG * mG * mG * mA * mG | | XXXXX XXXX |
| WV- | mU * Geom5CeoAeomC * A * C * A * G * T * A * G * A * T | UGCACACAGTAGATGAGGGA | XOOOX XXXXX |
| 11120 | * G * mA * mG * mG * mG * mA | | XXXXX XXXX |
| WV- | mA * GeoTeoGeomC * A * C * A * C * A * G * T * A * G * A | AGTGCACACAGTAGAUGAGG | XOOOX XXXXX |
| 11121 | * mU * mG * mA * mG * mG | | XXXXX XXXX |
| WV- | mA * AeoGeoTeomG * C * A * C * A * C * A * G * T * A * G | AAGTGCACACAGTAGAUGAG | XOOOX XXXXX |
| 11122 | * mA * mU * mG * mA * mG | | XXXXX XXXX |
| WV- | mG * AeoAeoGeomU * G * C * A * C * A * C * A * G * T * A | GAAGUGCACACAGTAGAUGA | XOOOX XXXXX |
| 11123 | * mG * mA * mU * mG * mA | | XXXXX XXXX |
| WV- | mA * Sm5CeoAeoGeomU * SA * SG * RA * ST * SG * SA * | ACAGUAGATGAGGGAGGAGG | SOOOS SRSSS SSSSS |
| 11124 | SG * SG * SG * SA * SmG * SmG * SmA * SmG * SmG | | SSSS |
| WV- | mC * SAeom5CeoAeomG * ST * SA * SG * RA * ST * SG * | CACAGTAGATGAGGGAGGAG | SOOOS SSRSS SSSSS |
| 11125 | SA * SG * SG * SG * SmA * SmG * SmG * SmA * SmG | | SSSS |
| WV- | mA * Sm5CeoAeom5CeomA * SG * ST * SA * SG * RA * ST | ACACAGTAGATGAGGGAGGA | SOOOS SSSRS SSSSS |
| 11126 | * SG * SA * SG * SG * SmG * SmA * SmG * SmG * SmA | | SSSS |
| WV- | mC * SAeom5CeoAeomC * SA * SG * ST * SA * SG * RA * | CACACAGTAGATGAGGGAGG | SOOOS SSSSR SSSSS |
| 11127 | ST * SG * SA * SG * SmG * SmG * SmA * SmG * SmG | | SSSS |
| WV- | mG * Sm5CeoAeom5CeomA * SC * SA * SG * ST * SA * SG | GCACACAGTAGATGAGGGAG | SOOOS SSSSS RSSSS |
| 11128 | * RA * ST * SG * SA * SmG * SmG * SmG * SmA * SmG | | SSSS |
| WV- | mU * SGeom5CeoAeomC * SA * SC * SA * SG * ST * SA * | UGCACACAGTAGATGAGGGA | SOOOS SSSSS SRSSS |
| 11129 | SG * RA * ST * SG * SmA * SmG * SmG * SmG * SmA | | SSSS |
| WV- | mA * SGeoTeoGeomC * SA * SC * SA * SC * SA * SG * ST * | AGTGCACACAGTAGAUGAGG | SOOOS SSSSS SSSRS |
| 11130 | SA * SG * RA * SmU * SmG * SmA * SmG * SmG | | SSSS |
| WV- | mA * SAeoGeoTeomG * SC * SA * SC * SA * SC * SA * SG * | AAGTGCACACAGTAGAUGAG | SOOOS SSSSS SSSSR |
| 11131 | ST * SA * SG * RmA * SmU * SmG * SmA * SmG | | SSSS |
| WV- | mG * SAeoAeoGeomU * SG * SC * SA * SC * SA * SC * SA * | GAAGUGCACACAGTAGAUGA | SOOOS SSSSS SSSSS |
| 11132 | SG * ST * SA * SmG * RmA * SmU * SmG * SmA | | RSSS |
| WV- | T * fU * mAmG * mGmA * mGmG * mCmA * mGmA * | TUAGGAGGCAGAAGCAGACGUTU | XXOXO XOXOX |
| 11213 | mAfG * mC * mA * mG * mA * mC * mG * mU * T * mU | | OXOXX XXXXX XX |
| WV- | Aeo * GeoGeoGeom5Ceo * G * C * A * G * A * C * T * T * C | AGGGCGCAGACTTCCAAAGG | XOOOX XXXXX |
| 11534 | * C * AeoAeoAeoGeo * Geo | | XXXXX OOOX |
| WV- | Aeo * AeoGeoGeoGeo * C * G * C * A * G * A * C * T * T * C | AAGGGCGCAGACTTCCAAAG | XOOOX XXXXX |
| 11535 | * m5CeoAeoAeoAeo * Geo | | XXXXX OOOX |
| WV- | m5Ceo * AeoAeoGeoGeo * G * C * G * C * A * G * A * C * T | CAAGGGCGCAGACTTCCAAA | XOOOX XXXXX |
| 11536 | * T * m5Ceom5CeoAeoAeo * Aeo | | XXXXX OOOX |
| WV- | Aeo * m5CeoAeoAeoGeo * G * G * C * G * C * A * G * A * C | ACAAGGGCGCAGACTTCCAA | XOOOX XXXXX |
| 11537 | * T * Teom5Ceom5CeoAeo * Aeo | | XXXXX OOOX |
| WV- | m5Ceo * Aeom5CeoAeoAeo * G * G * G * C * G * C * A * G | CACAAGGGCGCAGACTTCCA | XOOOX XXXXX |
| 11538 | * A * C * TeoTeom5Ceom5Ceo * Aeo | | XXXXX OOOX |
| WV- | Geo * m5CeoAeom5CeoAeo * A * G * G * G * C * G * C * A | GCACAAGGGCGCAGACTTCC | XOOOX XXXXX |
| 11539 | * G * A * m5CeoTeoTeom5Ceo * m5Ceo | | XXXXX OOOX |
| WV- | Geo * Geom5CeoAeom5Ceo * A * A * G * G * G * C * G * C | GGCACAAGGGCGCAGACTTC | XOOOX XXXXX |
| 11540 | * A * G * Aeom5CeoTeoTeo * m5Ceo | | XXXXX OOOX |
| WV- | Geo * GeoGeom5CeoAeo * C * A * A * G * G * G * C * G * C | GGGCACAAGGGCGCAGACTT | XOOOX XXXXX |
| 11541 | * A * GeoAeom5CeoTeo * Teo | | XXXXX OOOX |
| WV- | Aeo * GeoGeoGeom5Ceo * A * C * A * A * G * G * G * C * G | AGGGCACAAGGGCGCAGACT | XOOOX XXXXX |
| 11542 | * C * AeoGeoAeom5Ceo * Teo | | XXXXX OOOX |
| WV- | m5Ceo * AeoGeoGeoGeo * C * A * C * A * A * G * G * G * C | CAGGGCACAAGGGCGCAGAC | XOOOX XXXXX |
| 11543 | * G * m5CeoAeoGeoAeo * m5Ceo | | XXXXX OOOX |
| WV- | mA * m5CeoAeoGeomU * G * G * A * T * G * A * G * G * G | ACAGUGGATGAGGGAGGAGG | XOOOX XXXXX |
| 11548 | * A * mG * mG * mA * mG * mG | | XXXXX XXXX |
| WV- | mC * Aeom5CeoAeomG * T * G * G * A * T * G * A * G * G | CACAGTGGATGAGGGAGGAG | XOOOX XXXXX |
| 11549 | * G * mA * mG * mG * mA * mG | | XXXXX XXXX |
| WV- | mA * m5CeoAeom5CeomA * G * T * G * G * A * T * G * A * | ACACAGTGGATGAGGGAGGA | XOOOX XXXXX |
| 11550 | G * G * mG * mA * mG * mG * mA | | XXXXX XXXX |
| WV- | mC * Aeom5CeoAeomC * A * G * T * G * G * A * T * G * A | CACACAGTGGATGAGGGAGG | XOOOX XXXXX |
| 11551 | * G * mG * mG * mA * mG * mG | | XXXXX XXXX |
| WV- | mG * m5CeoAeom5CeomA * C * A * G * T * G * G * A * T * | GCACACAGTGGATGAGGGAG | XOOOX XXXXX |
| 11552 | G * A * mG * mG * mG * mA * mG | | XXXXX XXXX |
| WV- | mU * Geom5CeoAeomC * A * C * A * G * T * G * G * A * T | UGCACACAGTGGATGAGGGA | XOOOX XXXXX |
| 11553 | * G * mA * mG * mG * mG * mA | | XXXXX XXXX |
| WV- | mG * TeoGeom5CeomA * C * A * C * A * G * T * G * G * A | GTGCACACAGTGGATGAGGG | XOOOX XXXXX |
| 11554 | * T * mG * mA * mG * mG * mG | | XXXXX XXXX |
| WV- | mA * GeoTeoGeomC * A * C * A * C * A * G * T * G * G * A | AGTGCACACAGTGGAUGAGG | XOOOX XXXXX |
| 11555 | * mU * mG * mA * mG * mG | | XXXXX XXXX |
| WV- | mA * AeoGeoTeomG * C * A * C * A * C * A * G * T * G * G | AAGTGCACACAGTGGAUGAG | XOOOX XXXXX |
| 11556 | * mA * mU * mG * mA * mG | | XXXXX XXXX |
| WV- | mG * AeoAeoGeomU * G * C * A * C * A * C * A * G * T * G | GAAGUGCACACAGTGGAUGA | XOOOX XXXXX |
| 11557 | * mG * mA * mU * mG * mA | | XXXXX XXXX |
| WV- | mA * Sm5CeoAeoGeomU * SG * SG * RA * ST * SG * SA * | ACAGUGGATGAGGGAGGAGG | SOOOS SRSSS SSSSS |
| 11558 | SG * SG * SG * SA * SmG * SmG * SmA * SmG * SmG | | SSSS |
| WV- | mC * SAeom5CeoAeomG * ST * SG * SG * RA * ST * SG * | CACAGTGGATGAGGGAGGAG | SOOOS SSRSS SSSSS |
| 11559 | SA * SG * SG * SG * SmA * SmG * SmG * SmA * SmG | | SSSS |
| WV- | mA * Sm5CeoAeom5CeomA * SG * ST * SG * SG * RA * ST | ACACAGTGGATGAGGGAGGA | SOOOS SSSRS SSSSS |
| 11560 | * SG * SA * SG * SG * SmG * SmA * SmG * SmG * SmA | | SSSS |
| WV- | mC * SAeom5CeoAeomC * SA * SG * ST * SG * SG * RA * | CACACAGTGGATGAGGGAGG | SOOOS SSSSR SSSSS |
| 11561 | ST * SG * SA * SG * SmG * SmG * SmA * SmG * SmG | | SSSS |
| WV- | mG * Sm5CeoAeom5CeomA * SC * SA * SG * ST * SG * SG | GCACACAGTGGATGAGGGAG | SOOOS SSSSS RSSSS |
| 11562 | * RA * ST * SG * SA * SmG * SmG * SmG * SmA * SmG | | SSSS |
| WV- | mU * SGeom5CeoAeomC * SA * SC * SA * SG * ST * SG * | UGCACACAGTGGATGAGGGA | SOOOS SSSSS SRSSS |
| 11563 | SG * RA * ST * SG * SmA * SmG * SmG * SmG * SmA | | SSSS |
| WV- | mG * STeoGeom5CeomA * SC * SA * SC * SA * SG * ST * | GTGCACACAGTGGATGAGGG | SOOOS SSSSS SSRSS |
| 11564 | SG * SG * RA * ST * SmG * SmA * SmG * SmG * SmG | | SSSS |
| WV- | mA * SGeoTeoGeomC * SA * SC * SA * SC * SA * SG * ST * | AGTGCACACAGTGGAUGAGG | SOOOS SSSSS SSSRS |
| 11565 | SG * SG * RA * SmU * SmG * SmA * SmG * SmG | | SSSS |
| WV- | mA * SAeoGeoTeomG * SC * SA * SC * SA * SC * SA * SG * | AAGTGCACACAGTGGAUGAG | SOOOS SSSSS SSSSR |
| 11566 | ST * SG * SG * RmA * SmU * SmG * SmA * SmG | | SSSS |
| WV- | mG * SAeoAeoGeomU * SG * SC * SA * SC * SA * SC * SA * | GAAGUGCACACAGTGGAUGA | SOOOS SSSSS SSSSS |
| 11567 | SG * ST * SG * SmG * RmA * SmU * SmG * SmA | | RSSS |
| WV- | mU * mU * mc * mU * C * T * A * T * T * G * C * A * C * A | UUCUCTATTGCACAUUCC | XXXXX XXXXX |
| 11568 | * mU * mU * mC * mC | | XXXXX XX |
| WV- | Mod062L008mU * mU * mc * mU * C * T * A * T * T * G * C | UUCUCTATTGCACAUUCC | OXXXXXXXXX |
| 11569 | * A * C * A * mU * mU * mC * mC | | XXXXXXXX |
| WV- | mU * mU * mc * mU * C * T * A * T * T * G * C * A * C * A | UUCUCTATTGCACAUUCC | XXXXX XXXXX |
| 11570 | * mU * mU * mc * mCMod094 | | XXXXX XXO |
| WV- | L008mU * mU * mc * mU * C * T * A * T * T * G * C * A * C | UUCUCTATTGCACAUUCC | OXXXXXXXXX |
| 11571 | * A * mU * mU * mC * mC | | XXXXXXXX |
| WV- | 1A * Geo * Geo * Geo * m5Ceo * G * C * A * G * A * C * T * T * C | AGGGCGCAGACTTCCAAAGG | XXXXX XXXXX |
| 11968 | * C * Aeo * Aeo * Aeo * Geo * 1G | | XXXXX XXXX |
| WV- | 1A * Aeo * Geo * Geo * Geo * C * G * C * A * G * A * C * T * T * | AAGGGCGCAGACTTCCAAAG | XXXXX XXXXX |
| 11969 | C * m5Ceo * Aeo * Aeo * Aeo * 1G | | XXXXX XXXX |
| WV- | m51C * Aeo * Aeo * Geo * Geo * G * C * G * C * A * G * A * C * | CAAGGGCGCAGACTTCCAAA | XXXXX XXXXX |
| 11970 | T * T * m5Ceo * m5Ceo * Aeo * Aeo * 1A | | XXXXX XXXX |
| WV- | 1A * m5Ceo * Aeo * Aeo * Geo * G * G * C * G * C * A * G * A * | ACAAGGGCGCAGACTTCCAA | XXXXX XXXXX |
| 11971 | C * T * Teo * m5Ceo * m5Ceo * Aeo * 1A | | XXXXX XXXX |
| WV- | m51C * Aeo * m5Ceo * Aeo * Aeo * G * G * G * C * G * C * A * G | CACAAGGGCGCAGACTTCCA | XXXXX XXXXX |
| 11972 | * A * C * Teo * Teo * m5Ceo * m5Ceo * 1A | | XXXXX XXXX |
| WV- | 1G * m5Ceo * Aeo * m5Ceo * Aeo * A * G * G * G * C * G * C * A | GCACAAGGGCGCAGACTTCC | XXXXX XXXXX |
| 11973 | * G * A * m5Ceo * Teo * Teo * m5Ceo * m51C | | XXXXX XXXX |
| WV- | 1G * Geo * m5Ceo * Aeo * m5Ceo * A * A * G * G * G * C * G * C | GGCACAAGGGCGCAGACTTC | XXXXX XXXXX |
| 11974 | * A * G * Aeo * m5Ceo * Teo * Teo * m51C | | XXXXX XXXX |
| WV- | 1G * Geo * Geo * m5Ceo * Aeo * C * A * A * G * G * G * C * G * | GGGCACAAGGGCGCAGACTT | XXXXX XXXXX |
| 11975 | C * A * Geo * Aeo * m5Ceo * Teo * 1T | | XXXXX XXXX |
| WV- | 1A * Geo * Geo * Geo * m5Ceo * A * C * A * A * G * G * G * C * | AGGGCACAAGGGCGCAGACT | XXXXX XXXXX |
| 11976 | G * C * Aeo * Geo * Aeo * m5Ceo * 1T | | XXXXX XXXX |
| WV- | m51C * Aeo * Geo * Geo * Geo * C * A * C * A * A * G * G * G * | CAGGGCACAAGGGCGCAGAC | XXXXX XXXXX |
| 11977 | C * G * m5Ceo * Aeo * Geo * Aeo * m51C | | XXXXX XXXX |
| WV- | 1A * 1G *1G * 1G * m51C * G * C * A * G * A * C * T * T * C * C * | AGGGCGCAGACTTCCAAAGG | XXXXX XXXXX |
| 11978 | 1A * 1A *1A * 1G * 1G | | XXXXX XXXX |
| WV- | 1A * 1A * 1G *1G * 1G * C * G * C * A * G * A * C * T * T * C * | AAGGGCGCAGACTTCCAAAG | XXXXX XXXXX |
| 11979 | m51C * 1A * 1A * 1A * 1G | | XXXXX XXXX |
| WV- | m51C * 1A * 1A * 1G * 1G * G * C * G * C * A * G * A * C * T * T * | CAAGGGCGCAGACTTCCAAA | XXXXX XXXXX |
| 11980 | m51C * m51C * 1A * 1A * 1A | | XXXXX XXXX |
| WV- | 1A * m51C * 1A * 1A *1G * G * G * C * G * C * A * G * A * C * T * | ACAAGGGCGCAGACTTCCAA | XXXXX XXXXX |
| 11981 | 1T * m51C * m51C * 1A * 1A | | XXXXX XXXX |
| WV- | m51C * 1A * m51C * 1A * 1A * G * G * G * C * G * C * A * G * A * | CACAAGGGCGCAGACTTCCA | XXXXX XXXXX |
| 11982 | C * 1T * 1T * m51C * m51C * 1A | | XXXXX XXXX |
| WV- | 1G * m51C *1A * m51C *1A * A * G * G * G * C * G * C * A * G * | GCACAAGGGCGCAGACTTCC | XXXXX XXXXX |
| 11983 | A * m51C * 1T * 1T * m51C * m51C | | XXXXX XXXX |
| WV- | 1G * 1G * m51C * 1A * m51C * A * A * G * G * G * C * G * C * A * | GGCACAAGGGCGCAGACTTC | XXXXX XXXXX |
| 11984 | G * 1A * m51C * 1T * 1T * m51C | | XXXXX XXXX |
| WV- | 1G * 1G * 1G * m51C * 1A * C * A * A * G * G * G * C * G * C * A * | GGGCACAAGGGCGCAGACTT | XXXXX XXXXX |
| 11985 | 1G * 1A * m51C * 1T * 1T | | XXXXX XXXX |
| WV- | 1A * 1G * 1G * 1G * m51C * A * C * A * A * G * G * G * C * G * C * | AGGGCACAAGGGCGCAGACT | XXXXX XXXXX |
| 11986 | 1A * 1G * 1A * m51C * 1T | | XXXXX XXXX |
| WV- | m51C * 1A * 1G * 1G * 1G * C * A * C * A * A * G * G * G * C * G * | CAGGGCACAAGGGCGCAGAC | XXXXX XXXXX |
| 11987 | m51C * 1A * 1G * 1A * m51C | | XXXXX XXXX |
| WV- | 1A * GeoGeoGeom5Ceo * G * C * A * G * A * C * T * T * C * C * | AGGGCGCAGACTTCCAAAGG | XOOOX XXXXX |
| 11988 | mA * mA * mA * mG * 1G | | XXXXX XXXX |
| WV- | 1A * AeoGeoGeoGeo * C * G * C * A * G * A * C * T * T * C * mC | AAGGGCGCAGACTTCCAAAG | XOOOX XXXXX |
| 11989 | * mA * mA * mA * 1G | | XXXXX XXXX |
| WV- | m51C * AeoAeoGeoGeo * G * C * G * C * A * G * A * C * T * T * | CAAGGGCGCAGACTTCCAAA | XOOOX XXXXX |
| 11990 | mC * mC * mA * mA * 1A | | XXXXX XXXX |
| WV- | 1A * m5CeoAeoAeoGeo * G * G * C * G * C * A * G * A * C * T * | ACAAGGGCGCAGACTUCCAA | XOOOX XXXXX |
| 11991 | mU * mC * mC * mA * 1A | | XXXXX XXXX |
| WV- | m51C * Aeom5CeoAeoAeo * G * G * G * C * G * C * A * G * A * | CACAAGGGCGCAGACUUCCA | XOOOX XXXXX |
| 11992 | C * mU * mU * mC * mC * 1A | | XXXXX XXXX |
| WV- | 1G * m5CeoAeom5CeoAeo * A * G * G * G * C * G * C * A * G * | GCACAAGGGCGCAGACUUCC | XOOOX XXXXX |
| 11993 | A * mC * mU * mU * mC * m51C | | XXXXX XXXX |
| WV- | 1G * Geom5CeoAeom5Ceo * A * A * G * G * G * C * G * C * A * | GGCACAAGGGCGCAGACUUC | XOOOX XXXXX |
| 11994 | G * mA * mC * mU * mU * m51C | | XXXXX XXXX |
| WV- | 1G * GeoGeom5CeoAeo * C * A * A * G * G * G * C * G * C * A * | GGGCACAAGGGCGCAGACUT | XOOOX XXXXX |
| 11995 | mG * mA * mC * mU * 1T | | XXXXX XXXX |
| WV- | 1A * GeoGeoGeom5Ceo * A * C * A * A * G * G * G * C * G * C * | AGGGCACAAGGGCGCAGACT | XOOOX XXXXX |
| 11996 | mA * mG * mA * mC * 1T | | XXXXX XXXX |
| WV- | m51C * AeoGeoGeoGeo * C * A * C * A * A * G * G * G * C * G * | CAGGGCACAAGGGCGCAGAC | XOOOX XXXXX |
| 11997 | mC * mA * mG * mA * m51C | | XXXXX XXXX |
| WV- | 1A * GeoGeoGeom5Ceo * G * C * A * G * A * C * T * T * C * C * | AGGGCGCAGACTTCCAAAGG | XOOOX XXXXX |
| 11998 | Aeo * AeoAeoGeo * 1G | | XXXXX XOOX |
| WV- | 1A * AeoGeoGeoGeo * C * G * C * A * G * A * C * T * T * C * | AAGGGCGCAGACTTCCAAAG | XOOOX XXXXX |
| 11999 | m5Ceo * AeoAeoAeo * 1G | | XXXXX XOOX |
| WV- | m51C * AeoAeoGeoGeo * G * C * G * C * A * G * A * C * T * T * | CAAGGGCGCAGACTTCCAAA | XOOOX XXXXX |
| 12000 | m5Ceo * m5CeoAeoAeo * 1A | | XXXXX XOOX |
| WV- | 1A * m5CeoAeoAeoGeo * G * G * C * G * C * A * G * A * C * T * | ACAAGGGCGCAGACTTCCAA | XOOOX XXXXX |
| 12001 | Teo * m5Ceom5CeoAeo * 1A | | XXXXX XOOX |
| WV- | m51C * Aeom5CeoAeoAeo * G * G * G * C * G * C * A * G * A * | CACAAGGGCGCAGACTTCCA | XOOOX XXXXX |
| 12002 | C * Teo * Teom5Ceom5Ceo * 1A | | XXXXX XOOX |
| WV- | 1G * m5CeoAeom5CeoAeo * A * G * G * G * C * G * C * A * G * | GCACAAGGGCGCAGACTTCC | XOOOX XXXXX |
| 12003 | A * m5Ceo * TeoTeom5Ceo * m51C | | XXXXX XOOX |
| WV- | 1G * Geom5CeoAeom5Ceo * A * A * G * G * G * C * G * C * A * | GGCACAAGGGCGCAGACTTC | XOOOX XXXXX |
| 12004 | G * Aeo * m5CeoTeoTeo * m51C | | XXXXX XOOX |
| WV- | 1G * GeoGeom5CeoAeo * C * A * A * G * G * G * C * G * C * A * | GGGCACAAGGGCGCAGACTT | XOOOX XXXXX |
| 12005 | Geo * Aeom5CeoTeo * 1T | | XXXXX XOOX |
| WV- | 1A * GeoGeoGeom5Ceo * A * C * A * A * G * G * G * C * G * C * | AGGGCACAAGGGCGCAGACT | XOOOX XXXXX |
| 12006 | Aeo * GeoAeom5Ceo * 1T | | XXXXX XOOX |
| WV- | m51C * AeoGeoGeoGeo * C * A * C * A * A * G * G * G * C * G * | CAGGGCACAAGGGCGCAGAC | XOOOX XXXXX |
| 12007 | m5Ceo * AeoGeoAeo * m51C | | XXXXX XOOX |
| WV- | POT * fG * mCfA * mGfA * mCfU * mUfC * mCfA * | TGCAGACUUCCAAAGGCUCCGTU | XXOXO XOXOX |
| 12008 | mAfA * mG * fG * mC * fU * mC * fC * mG * T * mU | | OXOXX XXXXX XX |
| WV- | POT * fC * mGfC * mAfG * mAfC * mUfU * mCfC * | TCGCAGACUUCCAAAGGCUCCTU | XXOXO XOXOX |
| 12009 | mAfA * mA * fG * mG * fC * mU * fC * mC * T * mU | | OXOXX XXXXX XX |
| WV- | POT * fG * mCfG * mCfA * mGfA * mCfU * mUfC * | TGCGCAGACUUCCAAAGGCUCTU | XXOXO XOXOX |
| 12010 | mCfA * mA * fA * mG * fG * mC * fU * mC * T * mU | | OXOXX XXXXX XX |
| WV- | POT * fG * mGfC * mGfC * mAfG * mAfC * mUfU * | TGGCGCAGACUUCCAAAGGCUTU | XXOXO XOXOX |
| 12011 | mCfC * mA * fA * mA * fG * mG * fC * mU * T * mU | | OXOXX XXXXX XX |
| WV- | POT * fG * mGfG * mCfG * mCfA * mGfA * mCfU * | TGGGCGCAGACUUCCAAAGGCTU | XXOXO XOXOX |
| 12012 | mUfC * mC * fA * mA * fA * mG * fG * mC * T * mU | | OXOXX XXXXX XX |
| WV- | POT * fA * mGfG * mGfC * mGfC * mAfG * mAfC * | TAGGGCGCAGACUUCCAAAGGTU | XXOXO XOXOX |
| 12013 | mUfU * mC * fC * mA * fA * mA * fG * mG * T * mU | | OXOXX XXXXX XX |
| WV- | POT * fA * mAfG * mGfG * mCfG * mCfA * mGfA * | TAAGGGCGCAGACUUCCAAAGTU | XXOXO XOXOX |
| 12014 | mCfU * mU * fC * mC * fA * mA * fA * mG * T * mU | | OXOXX XXXXX XX |
| WV- | POT * fC * mAfA * mGfG * mGfC * mGfC * mAfG * | TCAAGGGCGCAGACUUCCAAATU | XXOXO XOXOX |
| 12015 | mAfC * mU * fU * mC * fC * mA * fA * mA * T * mU | | OXOXX XXXXX XX |
| WV- | POT * fA * mCfA * mAfG * mGfG * mCfG * mCfA * | TACAAGGGCGCAGACUUCCAATU | XXOXO XOXOX |
| 12016 | mGfA * mC * fU * mU * fC * mC * fA * mA * T * mU | | OXOXX XXXXX XX |
| WV- | POT * fC * mAfC * mAfA * mGfG * mGfC * mGfC * | TCACAAGGGCGCAGACUUCCATU | XXOXO XOXOX |
| 12017 | mAfG * mA * fC * mU * fU * mC * fC * mA * T * mU | | OXOXX XXXXX XX |
| WV- | POT * fG * mCfA * mCfA * mAfG * mGfG * mCfG * | TGCACAAGGGCGCAGACUUCCTU | XXOXO XOXOX |
| 12018 | mCfA * mG * fA * mC * fU * mU * fC * mC * T * mU | | OXOXX XXXXX XX |
| WV- | POT * fG * mGfC * mAfC * mAfA * mGfG * mGfC * | TGGCACAAGGGCGCAGACUUCTU | XXOXO XOXOX |
| 12019 | mGfC * mA * fG * mA * fC * mU * fU * mC * T * mU | | OXOXX XXXXX XX |
| WV- | POT * fG * mGfG * mCfA * mCfA * mAfG * mGfG * | TGGGCACAAGGGCGCAGACUUTU | XXOXO XOXOX |
| 12020 | mCfG * mC * fA * mG * fA * mC * fU * mU * T * mU | | OXOXX XXXXX XX |
| WV- | POT * fA * mGfG * mGfC * mAfC * mAfA * mGfG * | TAGGGCACAAGGGCGCAGACUTU | XXOXO XOXOX |
| 12021 | mGfC * mG * fC * mA * fG * mA * fC * mU * T * mU | | OXOXX XXXXX XX |
| WV- | POT * fC * mAfG * mGfG * mCfA * mCfA * mAfG * | TCAGGGCACAAGGGCGCAGACTU | XXOXO XOXOX |
| 12022 | mGfG * mC * fG * mC * fA * mG * fA * mC * T * mU | | OXOXX XXXXX XX |
| WV- | POT * fG * mCfA * mGfG * mGfC * mAfC * mAfA * | TGCAGGGCACAAGGGCGCAGATU | XXOXO XOXOX |
| 12023 | mGfG * mG * fC * mG * fC * mA * fG * mA * T * mU | | OXOXX XXXXX XX |
| WV- | POT * fG * mGfC * mAfG * mGfG * mCfA * mCfA * | TGGCAGGGCACAAGGGCGCAGTU | XXOXO XOXOX |
| 12024 | mAfG * mG * fG * mC * fG * mC * fA * mG * T * mU | | OXOXX XXXXX XX |
| WV- | POT * fA * mGfG * mCfA * mGfG * mGfC * mAfC * | TAGGCAGGGCACAAGGGCGCATU | XXOXO XOXOX |
| 12025 | mAfA * mG * fG * mG * fC * mG * fC * mA * T * mU | | OXOXX XXXXX XX |
| WV- | POT * fG * mAfG * mGfC * mAfG * mGfG * mCfA * | TGAGGCAGGGCACAAGGGCGCTU | XXOXO XOXOX |
| 12026 | mCfA * mA * fG * mG * fG * mC * fG * mC * T * mU | | OXOXX XXXXX XX |
| WV- | POT * fG * mGfA * mGfG * mCfA * mGfG * mGfC * | TGGAGGCAGGGCACAAGGGCGTU | XXOXO XOXOX |
| 12027 | mAfC * mA * fA * mG * fG * mG * fC * mG * T * mU | | OXOXX XXXXX XX |
| WV- | POT * fG * mCfA * mGfA * mCfU * mUfC * mCfA * | TGCAGACUUCCAAAGGCUCCGTU | XXOXO XOXOX |
| 12028 | mAfA * mGfGmCfUmCfCmG * T * mU | | OXOXO OOOOO XX |
| WV- | POT * fC * mGfC * mAfG * mAfC * mUfU * mCfC * | TCGCAGACUUCCAAAGGCUCCTU | XXOXO XOXOX |
| 12029 | mAfA * mAfGmGfCmUfCmC * T * mU | | OXOXO OOOOO XX |
| WV- | POT * fG * mCfG * mCfA * mGfA * mCfU * mUfC * | TGCGCAGACUUCCAAAGGCUCTU | XXOXO XOXOX |
| 12030 | mCfA * mAfAmGfGmCfUmC * T * mU | | OXOXO OOOOO XX |
| WV- | POT * fG * mGfC * mGfC * mAfG * mAfC * mUfU * | TGGCGCAGACUUCCAAAGGCUTU | XXOXO XOXOX |
| 12031 | mCfC * mAfAmAfGmGfCmU * T * mU | | OXOXO OOOOO XX |
| WV- | POT * fG * mGfG * mCfG * mCfA * mGfA * mCfU * | TGGGCGCAGACUUCCAAAGGCTU | XXOXO XOXOX |
| 12032 | mUfC * mCfAmAfAmGfGmC * T * mU | | OXOXO OOOOO XX |
| WV- | POT * fA * mGfG * mGfC * mGfC * mAfG * mAfC * | TAGGGCGCAGACUUCCAAAGGTU | XXOXO XOXOX |
| 12033 | mUfU * mCfCmAfAmAfGmG * T * mU | | OXOXO OOOOO XX |
| WV- | POT * fA * mAfG * mGfG * mCfG * mCfA * mGfA * | TAAGGGCGCAGACUUCCAAAGTU | XXOXO XOXOX |
| 12034 | mCfU * mUfCmCfAmAfAmG * T * mU | | OXOXO OOOOO XX |
| WV- | POT * fC * mAfA * mGfG * mGfC * mGfC * mAfG * | TCAAGGGCGCAGACUUCCAAATU | XXOXO XOXOX |
| 12035 | mAfC * mUfUmCfCmAfAmA * T * mU | | OXOXO OOOOO XX |
| WV- | POT * fA * mCfA * mAfG * mGfG * mCfG * mCfA * | TACAAGGGCGCAGACUUCCAATU | XXOXO XOXOX |
| 12036 | mGfA * mCfUmUfCmCfAmA * T * mU | | OXOXO OOOOO XX |
| WV- | POT * fC * mAfC * mAfA * mGfG * mGfC * mGfC * | TCACAAGGGCGCAGACUUCCATU | XXOXO XOXOX |
| 12037 | mAfG * mAfCmUfUmCfCmA * T * mU | | OXOXO OOOOO XX |
| WV- | POT * fG * mCfA * mCfA * mAfG * mGfG * mCfG * | TGCACAAGGGCGCAGACUUCCTU | XXOXO XOXOX |
| 12038 | mCfA * mGfAmCfUmUfCmC * T * mU | | OXOXO OOOOO XX |
| WV- | POT * fG * mGfC * mAfC * mAfA * mGfG * mGfC * | TGGCACAAGGGCGCAGACUUCTU | XXOXO XOXOX |
| 12039 | mGfC * mAfGmAfCmUfUmC * T * mU | | OXOXO OOOOO XX |
| WV- | POT * fG * mGfG * mCfA * mCfA * mAfG * mGfG * | TGGGCACAAGGGCGCAGACUUTU | XXOXO XOXOX |
| 12040 | mCfG * mCfAmGfAmCfUmU * T * mU | | OXOXO OOOOO XX |
| WV- | POT * fA * mGfG * mGfC * mAfC * mAfA * mGfG * | TAGGGCACAAGGGCGCAGACUTU | XXOXO XOXOX |
| 12041 | mGfC * mGfCmAfGmAfCmU * T * mU | | OXOXO OOOOO XX |
| WV- | POT * fC * mAfG * mGfG * mCfA * mCfA * mAfG * | TCAGGGCACAAGGGCGCAGACTU | XXOXO XOXOX |
| 12042 | mGfG * mCfGmCfAmGfAmC * T * mU | | OXOXO OOOOO XX |
| WV- | POT * fG * mCfA * mGfG * mGfC * mAfC * mAfA * | TGCAGGGCACAAGGGCGCAGATU | XXOXO XOXOX |
| 12043 | mGfG * mGfCmGfCmAfGmA * T * mU | | OXOXO OOOOO XX |
| WV- | POT * fG * mGfC * mAfG * mGfG * mCfA * mCfA * | TGGCAGGGCACAAGGGCGCAGTU | XXOXO XOXOX |
| 12044 | mAfG * mGfGmCfGmCfAmG * T * mU | | OXOXO OOOOO XX |
| WV- | POT * fA * mGfG * mCfA * mGfG * mGfC * mAfC * | TAGGCAGGGCACAAGGGCGCATU | XXOXO XOXOX |
| 12045 | mAfA * mGfGmGfCmGfCmA * T * mU | | OXOXO OOOOO XX |
| WV- | POT * fG * mAfG * mGfC * mAfG * mGfG * mCfA * | TGAGGCAGGGCACAAGGGCGCTU | XXOXO XOXOX |
| 12046 | mCfA * mAfGmGfGmCfGmC * T * mU | | OXOXO OOOOO XX |
| WV- | POT * fG * mGfA * mGfG * mCfA * mGfG * mGfC * | TGGAGGCAGGGCACAAGGGCGTU | XXOXO XOXOX |
| 12047 | mAfC * mAfAmGfGmGfCmG * T * mU | | OXOXO OOOOO XX |
| WV- | rC rC rC rU rC rA rU rC rU rA rC rU rG rU rG rU rG rC rA rC | CCCUCAUCUACUGUGUGCAC | OOOOO OOOOO |
| 12049 | | | OOOOO OOOO |
| WV- | m5Ceo * m5CeoTeom5CeoAeo * G * G * C * C * C * C * C * A * | CCTCAGGCCCCCAGGTTACC | XOOOX XXXXX |
| 12213 | G * G * TeoTeoAeom5Ceo * m5Ceo | | XXXXX OOOX |
| WV- | Geo * STeo * STeo * SGeo * SAeo * RT * RC * ST * SG * ST * SA | GTTGATCTGTAGCAGCAGCT | SSSSR RSSSS SSSSS |
| 12258 | * SG * SC * SA * SG * Sm5Ceo * SAeo * SGeo * Sm5Ceo * STeo | | SSSS |
| WV- | Geo * STeo * STeo * SGeo * SAeo * RT * SC * RT * SG * ST * SA | GTTGATCTGTAGCAGCAGCT | SSSSR SRSSS SSSSS |
| 12259 | * SG * SC * SA * SG * Sm5Ceo * SAeo * SGeo * Sm5Ceo * STeo | | SSSS |
| WV- | Geo * STeo * STeo * SGeo * SAeo * RT * SC * ST * RG * ST * SA | GTTGATCTGTAGCAGCAGCT | SSSSR SSRSS SSSSS |
| 12260 | * SG * SC * SA * SG * Sm5Ceo * SAeo * SGeo * Sm5Ceo * STeo | | SSSS |
| WV- | Geo * STeo * STeo * SGeo * SAeo * RT * SC * ST * SG * RT * SA | GTTGATCTGTAGCAGCAGCT | SSSSR SSSRS SSSSS |
| 12261 | * SG * SC * SA * SG * Sm5Ceo * SAeo * SGeo * Sm5Ceo * STeo | | SSSS |
| WV- | Geo * STeo * STeo * SGeo * SAeo * RT * SC * ST * SG * ST * RA | GTTGATCTGTAGCAGCAGCT | SSSSR SSSSR SSSSS |
| 12262 | * SG * SC * SA * SG * Sm5Ceo * SAeo * SGeo * Sm5Ceo * STeo | | SSSS |
| WV- | Geo * STeo * STeo * SGeo * SAeo * RT * SC * ST * SG * ST * SA | GTTGATCTGTAGCAGCAGCT | SSSSR SSSSS RSSSS |
| 12263 | * RG * SC * SA * SG * Sm5Ceo * SAeo * SGeo * Sm5Ceo * STeo | | SSSS |
| WV- | Geo * STeo * STeo * SGeo * SAeo * RT * SC * ST * SG * ST * SA | GTTGATCTGTAGCAGCAGCT | SSSSR SSSSS SRSSS |
| 12264 | * SG * RC * SA * SG * Sm5Ceo * SAeo * SGeo * Sm5Ceo * STeo | | SSSS |
| WV- | Geo * STeo * STeo * SGeo * SAeo * RT * SC * ST * SG * ST * SA | GTTGATCTGTAGCAGCAGCT | SSSSR SSSSS SSRSS |
| 12265 | * SG * SC * RA * SG * Sm5Ceo * SAeo * SGeo * Sm5Ceo * STeo | | SSSS |
| WV- | Geo * STeo * STeo * SGeo * SAeo * RT * SC * ST * SG * ST * SA | GTTGATCTGTAGCAGCAGCT | SSSSR SSSSS SSSRS |
| 12266 | * SG * SC * SA * RG * Sm5Ceo * SAeo * SGeo * Sm5Ceo * STeo | | SSSS |
| WV- | Geo * STeo * STeo * SGeo * SAeo * RT * SC * ST * SG * ST * SA | GTTGATCTGTAGCAGCAGCT | SSSSR SSSSS SSSSR |
| 12267 | * SG * SC * SA * SG * Rm5Ceo * SAeo * SGeo * Sm5Ceo * STeo | | SSSS |
| WV- | Geo * STeoTeoGeoAeo * RT * RC * ST * SG * ST * SA * SG * SC | GTTGATCTGTAGCAGGCAGC | SOOOR RSSSS |
| 12268 | * SA * SG * SmG * SmC * SmA * SmG * SmC | | SSSSS SSSS |
| WV- | Geo * STeoTeoGeoAeo * RT * SC * RT * SG * ST * SA * SG * SC | GTTGATCTGTAGCAGGCAGC | SOOOR SRSSS |
| 12269 | * SA * SG * SmG * SmC * SmA * SmG * SmC | | SSSSS SSSS |
| WV- | Geo * STeoTeoGeoAeo * RT * SC * ST * RG * ST * SA * SG * SC | GTTGATCTGTAGCAGGCAGC | SOOOR SSRSS |
| 12270 | * SA * SG * SmG * SmC * SmA * SmG * SmC | | SSSSS SSSS |
| WV- | Geo * STeoTeoGeoAeo * RT * SC * ST * SG * RT * SA * SG * SC | GTTGATCTGTAGCAGGCAGC | SOOOR SSSRS |
| 12271 | * SA * SG * SmG * SmC * SmA * SmG * SmC | | SSSSS SSSS |
| WV- | Geo * STeoTeoGeoAeo * RT * SC * ST * SG * ST * RA * SG * SC | GTTGATCTGTAGCAGGCAGC | SOOOR SSSSR |
| 12272 | * SA * SG * SmG * SmC * SmA * SmG * SmC | | SSSSS SSSS |
| WV- | Geo * STeoTeoGeoAeo * RT * SC * ST * SG * ST * SA * RG * SC | GTTGATCTGTAGCAGGCAGC | SOOOR SSSSS |
| 12273 | * SA * SG * SmG * SmC * SmA * SmG * SmC | | RSSSS SSSS |
| WV- | Geo * STeoTeoGeoAeo * RT * SC * ST * SG * ST * SA * SG * RC | GTTGATCTGTAGCAGGCAGC | SOOOR SSSSS |
| 12274 | * SA * SG * SmG * SmC * SmA * SmG * SmC | | SRSSS SSSS |
| WV- | Geo * STeoTeoGeoAeo * RT * SC * ST * SG * ST * SA * SG * SC | GTTGATCTGTAGCAGGCAGC | SOOOR SSSSS |
| 12275 | * RA * SG * SmG * SmC * SmA * SmG * SmC | | SSRSS SSSS |
| WV- | Geo * STeoTeoGeoAeo * RT * SC * ST * SG * ST * SA * SG * SC | GTTGATCTGTAGCAGGCAGC | SOOOR SSSSS |
| 12276 | * SA * RG * SmG * SmC * SmA * SmG * SmC | | SSSRS SSSS |
| WV- | Geo * STeoTeoGeoAeo * RT * SC * ST * SG * ST * SA * SG * SC | GTTGATCTGTAGCAGGCAGC | SOOOR SSSSS |
| 12277 | * SA * SG * RmG * SmC * SmA * SmG * SmC | | SSSSR SSSS |
| WV- | mG * SmU * SmU * SmG * SmA * ST * RC * ST * SG * ST * SA * | GUUGATCTGTAGCAGCAGCT | SSSSS RSSSS SSSSR |
| 12278 | SG * SC * SA * SG * Rm5CeoAeoGeom5Ceo * STeo | | OOOS |
| WV- | mG * SmU * SmU * SmG * SmA * ST * SC * RT * SG * ST * SA * | GUUGATCTGTAGCAGCAGCT | SSSSS SRSSS SSSSR |
| 12279 | SG * SC * SA * SG * Rm5CeoAeoGeom5Ceo * STeo | | OOOS |
| WV- | mG * SmU * SmU * SmG * SmA * ST * SC * ST * RG * ST * SA * | GUUGATCTGTAGCAGCAGCT | SSSSS SSRSS SSSSR |
| 12280 | SG * SC * SA * SG * Rm5CeoAeoGeom5Ceo * STeo | | OOOS |
| WV- | mG * SmU * SmU * SmG * SmA * ST * SC * ST * SG * RT * SA * | GUUGATCTGTAGCAGCAGCT | SSSSS SSSRS SSSSR |
| 12281 | SG * SC * SA * SG * Rm5CeoAeoGeom5Ceo * STeo | | OOOS |
| WV- | mG * SmU * SmU * SmG * SmA * ST * SC * ST * SG * ST * RA * | GUUGATCTGTAGCAGCAGCT | SSSSS SSSSR SSSSR |
| 12282 | SG * SC * SA * SG * Rm5CeoAeoGeom5Ceo * STeo | | OOOS |
| WV- | mG * SmU * SmU * SmG * SmA * ST * SC * ST * SG * ST * SA * | GUUGATCTGTAGCAGCAGCT | SSSSS SSSSS RSSSR |
| 12283 | RG * SC * SA * SG * Rm5CeoAeoGeom5Ceo * STeo | | OOOS |
| WV- | mG * SmU * SmU * SmG * SmA * ST * SC * ST * SG * ST * SA * | GUUGATCTGTAGCAGCAGCT | SSSSS SSSSS SRSSR |
| 12284 | SG * RC * SA * SG * Rm5CeoAeoGeom5Ceo * STeo | | OOOS |
| WV- | mG * SmU * SmU * SmG * SmA * ST * SC * ST * SG * ST * SA * | GUUGATCTGTAGCAGCAGCT | SSSSS SSSSS SSRSR |
| 12285 | SG * SC * RA * SG * Rm5CeoAeoGeom5Ceo * STeo | | OOOS |
| WV- | mG * SmU * SmU * SmG * SmA * ST * SC * ST * SG * ST * SA * | GUUGATCTGTAGCAGCAGCT | SSSSS SSSSS |
| 12286 | SG * SC * SA * RG * Rm5CeoAeoGeom5Ceo * STeo | | SSSRROOOS |
| WV- | mG * SmU * SmU * SmG * SmA * ST * SC * ST * SG * ST * SA * | GUUGATCTGTAGCAGCAGCT | SSSSS SSSSS SSSSR |
| 12287 | SG * SC * SA * SG * Rm5CeoAeoGeom5Ceo * STeo | | OOOS |
| WV- | Geo * SGeom5CeoAeom5Ceo * RA * SA * SG * SG * SG * SC * SG * | GGCACAAGGGCGCAGACTTC | SOOOR SSSSS |
| 12288 | SC * RA * SG * SAeom5CeoTeoTeo * Sm5Ceo | | SSRSS OOOS |
| WV- | mG * SGeom5CeoAeom5Ceo * RA * SA * SG * SG * SG * SC * SG * | GGCACAAGGGCGCAGACUUC | SOOOR SSSSS |
| 12289 | SC * RA * SG * SmA * SmC * SmU * SmU * SmC | | SSRSS SSSS |
| WV- | Geo * SGeom5CeoAeom5Ceo * RA * SA * SG * SG * SG * SC * SG * | GGCACAAGGGCGCAGACUUC | SOOOR SSSSS |
| 12290 | SC * RA * SG * SmA * SmC * SmU * SmU * SmC | | SSRSS SSSS |
| WV- | mG * SmG * SmC * SmA * SmC * SA * SA * SG * SG * SG * SC * SG | GGCACAAGGGCGCAGACTTC | SSSSS SSSSS |
| 12291 | * SC * RA * SG * SAeom5CeoTeoTeo * Sm5Ceo | | SSRSS OOOS |
| WV- | mG * SmG * SmC * SmA * SmC * RA * SA * SG * SG * SG * SC * SG | GGCACAAGGGCGCAGACTTC | SSSSR SSSSS |
| 12292 | * SC * RA * SG * SAeom5CeoTeoTeo * Sm5Ceo | | SSRSS OOOS |
| WV- | mG * SmG * SmC * SmA * 5m5Ceo * RA * SA * SG * SG * SG * SC * | GGCACAAGGGCGCAGACTTC | SSSSR SSSSS |
| 12293 | SG * SC * RA * SG * SAeom5CeoTeoTeo * 5m5Ceo | | SSRSS OOOS |
| WV- | Geo * SGeom5CeoAeom5Ceo * RA * SA * SG * SG * SG * SC * SG * | GGCACAAGGGCGCAGACUUC | SOOOR SSSSS |
| 12294 | SC * RA * SG * SfA * SfC * SfU * SfU * SfC | | SSRSS SSSS |
| WV- | fG * SfG * SfC * SfA * SfC * SA * SA * SG * SG * SG * SC * SG * SC | GGCACAAGGGCGCAGACTTC | SSSSS SSSSS |
| 12295 | * RA * SG * SAeom5CeoTeoTeo * Sm5Ceo | | SSRSS OOOS |
| WV- | fG * SfG * SfC * SfA * SfC * RA * SA * SG * SG * SG * SC * SG * SC | GGCACAAGGGCGCAGACTTC | SSSSR SSSSS |
| 12296 | * RA * SG * SAeom5CeoTeoTeo * Sm5Ceo | | SSRSS OOOS |
| WV- | fG * SfG * SfC * SfA * Sm5Ceo * RA * SA * SG * SG * SG * SC * SG | GGCACAAGGGCGCAGACTTC | SSSSR SSSSS |
| 12297 | * SC * RA * SG * SAeom5CeoTeoTeo * Sm5Ceo | | SSRSS OOOS |
| WV- | fG * SfG * SfC * SfA * SfC * SA * SA * SG * SG * SG * SC * SG * SC | GGCACAAGGGCGCAGACUUC | SSSSS SSSSS |
| 12298 | * RA * SG * SmA * SmC * SmU * SmU * SmC | | SSRSS SSSS |
| WV- | fG * SfG * SfC * SfA * SfC * RA * SA * SG * SG * SG * SC * SG * SC | GGCACAAGGGCGCAGACUUC | SSSSR SSSSS |
| 12299 | * RA * SG * SmA * SmC * SmU * SmU * SmC | | SSRSS SSSS |
| WV- | mG * SmG * SmC * SmA * SmC * SA * SA * SG * SG * SG * SC * SG | GGCACAAGGGCGCAGACUUC | SSSSS SSSSS |
| 12300 | * SC * RA * SG * SfA * SfC * SfU * SfU * SfC | | SSRSS SSSS |
| WV- | mG * SmG * SmC * SmA * SmC * RA * SA * SG * SG * SG * SC * SG | GGCACAAGGGCGCAGACUUC | SSSSR SSSSS |
| 12301 | * SC * RA * SG * SfA * SfC * SfU * SfU * SfC | | SSRSS SSSS |
| WV- | mG * SmG * SmC * SmA * Sm5Ceo * RA * SA * SG * SG * SG * SC * | GGCACAAGGGCGCAGACUUC | SSSSR SSSSS |
| 12302 | SG * SC * RA * SG * SfA * SfC * SfU * SfU * SfC | | SSRSS SSSS |
| WV- | Geo * STeoTeoGeoAeo * RT * SC * ST * SG * ST * SA * RG * SC * | GTTGATCTGTAGCAGCAGCT | SOOOR SSSSS |
| 12425 | SA * SG * Sm5CeoAeoGeom5Ceo * STeo | | RSSSS OOOS |
| WV- | mG * STeoTeoGeoAeo * RT * SC * ST * SG * ST * SA * RG * SC * | GTTGATCTGTAGCAGCAGCU | SOOOR SSSSS |
| 12426 | SA * SG * SmC * SmA * SmG * SmC * SmU | | RSSSS SSSS |
| WV- | mG * SmU * SmU * SmG * SmA * ST * SC * ST * SG * ST * SA * RG | GUUGATCTGTAGCAGCAGCT | SSSSS SSSSS |
| 12427 | * SC * SA * SG * Sm5CeoAeoGeom5Ceo * STeo | | RSSSS OOOS |
| WV- | mG * SmU * SmU * SmG * SmA * RT * SC * ST * SG * ST * SA * RG | GUUGATCTGTAGCAGCAGCT | SSSSR SSSSS |
| 12428 | * SC * SA * SG * Sm5CeoAeoGeom5Ceo * STeo | | RSSSS OOOS |
| WV- | mG * SmU * SmU * SmG * SAeo * RT * SC * ST * SG * ST * SA * RG | GUUGATCTGTAGCAGCAGCT | SSSSR SSSSS |
| 12429 | * SC * SA * SG * Sm5CeoAeoGeom5Ceo * STeo | | RSSSS OOOS |
| WV- | Geo * STeoTeoGeoAeo * RT * SC * ST * SG * ST * SA * RG * SC * | GTTGATCTGTAGCAGCAGCU | SOOOR SSSSS |
| 12430 | SA * SG * SfC * SfA * SfG * SfC * SfU | | RSSSS SSSS |
| WV- | fG * SfU * SfU * SfG * SfA * ST * SC * ST * SG * ST * SA * RG * SC | GUUGATCTGTAGCAGCAGCT | SSSSS SSSSS |
| 12431 | * SA * SG * Sm5CeoAeoGeom5Ceo * STeo | | RSSSS OOOS |
| WV- | fG * SfU * SfU * SfG * SfA * RT * SC * ST * SG * ST * SA * RG * SC | GUUGATCTGTAGCAGCAGCT | SSSSR SSSSS |
| 12432 | * SA * SG * Sm5CeoAeoGeom5Ceo * STeo | | RSSSS OOOS |
| WV- | fG * SfU * SfU * SfG * SAeo * RT * SC * ST * SG * ST * SA * RG * | GUUGATCTGTAGCAGCAGCT | SSSSR SSSSS |
| 12433 | SC * SA * SG * Sm5CeoAeoGeom5Ceo * STeo | | RSSSS OOOS |
| WV- | fG * SfU * SfU * SfG * SfA * ST * SC * ST * SG * ST * SA * RG * SC | GUUGATCTGTAGCAGCAGCU | SSSSS SSSSS |
| 12434 | * SA * SG * SmC * SmA * SmG * SmC * SmU | | RSSSS SSSS |
| WV- | fG * SfU * SfU * SfG * SfA * RT * SC * ST * SG * ST * SA * RG * SC | GUUGATCTGTAGCAGCAGCU | SSSSR SSSSS |
| 12435 | * SA * SG * SmC * SmA * SmG * SmC * SmU | | RSSSS SSSS |
| WV- | mG * SmU * SmU * SmG * SmA * ST * SC * ST * SG * ST * SA * RG | GUUGATCTGTAGCAGCAGCU | SSSSS SSSSS |
| 12436 | * SC * SA * SG * SfC * SfA * SfG * SfC * SfU | | RSSSS SSSS |
| WV- | mG * SmU * SmU * SmG * SmA * RT * SC * ST * SG * ST * SA * RG | GUUGATCTGTAGCAGCAGCU | SSSSR SSSSS |
| 12437 | * SC * SA * SG * SfC * SfA * SfG * SfC * SfU | | RSSSS SSSS |
| WV- | mG * SmU * SmU * SmG * SAeo * RT * SC * ST * SG * ST * SA * RG | GUUGATCTGTAGCAGCAGCU | SSSSR SSSSS |
| 12438 | * SC * SA * SG * SfC * SfA * SfG * SfC * SFu | | RSSSS SSSS |
| WV- | Geo * SAeoGeom5CeoGeo * RG * SA * RG * SA * SA * SA * SC * SC | GAGCGGAGAAACCCTCCAAA | SOOOR SRSSS |
| 12509 | * SC * ST * Sm5Ceom5CeoAeoAeo * SAeo | | SSSSS OOOS |
| WV- | Teo * SGeoAeoGeom5Ceo * RG * SG * SA * RG * SA * SA * SA * SC | TGAGCGGAGAAACCCTCCAA | SOOOR SSRSS |
| 12510 | * SC * SC * STeom5Ceom5CeoAeo * SAeo | | SSSSS OOOS |
| WV- | m5Ceo * STeoGeoAeoGeo * RC * SG * SG * SA * RG * SA * SA * SA | CTGAGCGGAGAAACCCTCCA | SOOOR SSSRS |
| 12511 | * SC * SC * Sm5CeoTeom5Ceom5Ceo * SAeo | | SSSSS OOOS |
| WV- | Geo * Sm5CeoTeoGeoAeo * RG * SC * SG * SG * SA * RG * SA * SA | GCTGAGCGGAGAAACCCTCC | SOOOR SSSSR |
| 12512 | * SA * SC * Sm5Ceom5CeoTeom5Ceo * Sm5Ceo | | SSSSS OOOS |
| WV- | Geo * SGeom5CeoTeoGeo * RA * SG * SC * SG * SG * SA * RG * SA | GGCTGAGCGGAGAAACCCTC | SOOOR SSSSS |
| 12513 | * SA * SA * Sm5Ceom5Ceom5CeoTeo * Sm5Ceo | | RSSSS OOOS |
| WV- | Aeo * SGeoGeom5CeoTeo * RG * SA * SG * SC * SG * SG * SA * RG | AGGCTGAGCGGAGAAACCCT | SOOOR SSSSS |
| 12514 | * SA * SA * SAeom5Ceom5Ceom5Ceo * STeo | | SRSSS OOOS |
| WV- | Aeo * SAeoGeoGeom5Ceo * RT * SG * SA * SG * SC * SG * SG * SA | AAGGCTGAGCGGAGAAACCC | SOOOR SSSSS |
| 12515 | * RG * SA * SAeoAeom5Ceom5Ceo * Sm5Ceo | | SSRSS OOOS |
| WV- | m5Ceo * SAeoAeoGeoGeo * RC * ST * SG * SA * SG * SC * SG * SG | CAAGGCTGAGCGGAGAAACC | SOOOR SSSSS |
| 12516 | * SA * RG * SAeoAeoAeom5Ceo * Sm5Ceo | | SSSRS OOOS |
| WV- | m5Ceo * Sm5CeoAeoAeoGeo * RG * SC * ST * SG * SA * SG * SC * | CCAAGGCTGAGCGGAGAAAC | SOOOR SSSSS |
| 12517 | SG * SG * SA * RGeoAeoAeoAeo * Sm5Ceo | | SSSSR OOOS |
| WV- | Teo * Sm5Ceom5CeoAeoAeo * RG * SG * SC * ST * SG * SA * SG * | TCCAAGGCTGAGCGGAGAAA | SOOOR SSSSS |
| 12518 | SC * SG * SG * SAeoGeoAeoAeo * SAeo | | SSSSS OOOS |
| WV- | Geo * SAeoGeom5CeoGeo * RG * SA * RG * SA * SA * SA * SC * SC | GAGCGGAGAAACCCTCCAAA | SOOOR SRSSS |
| 12519 | * SC * ST * SmC * SmC * SmA * SmA * SmA | | SSSSS SSSS |
| WV- | Teo * SGeoAeoGeom5Ceo * RG * SG * SA * RG * SA * SA * SA * SC | TGAGCGGAGAAACCCUCCAA | SOOOR SSRSS |
| 12520 | * SC * SC * SmU * SmC * SmC * SmA * SmA | | SSSSS SSSS |
| WV- | m5Ceo * STeoGeoAeoGeo * RC * SG * SG * SA * RG * SA * SA * SA | CTGAGCGGAGAAACCCUCCA | SOOOR SSSRS |
| 12521 | * SC * SC * SmC * SmU * SmC * SmC * SmA | | SSSSS SSSS |
| WV- | Geo * Sm5CeoTeoGeoAeo * RG * SC * SG * SG * SA * RG * SA * SA | GCTGAGCGGAGAAACCCUCC | SOOOR SSSSR |
| 12522 | * SA * SC * SmC * SmC * SmU * SmC * SmC | | SSSSS SSSS |
| WV- | Geo * SGeom5CeoTeoGeo * RA * SG * SC * SG * SG * SA * RG * SA | GGCTGAGCGGAGAAACCCUC | SOOOR SSSSS |
| 12523 | * SA * SA * SmC * SmC * SmC * SmU * SmC | | RSSSS SSSS |
| WV- | Aeo * SGeoGeom5CeoTeo * RG * SA * SG * SC * SG * SG * SA * RG | AGGCTGAGCGGAGAAACCCU | SOOOR SSSSS |
| 12524 | * SA * SA * SmA * SmC * SmC * SmC * SmU | | SRSSS SSSS |
| WV- | Aeo * SAeoGeoGeom5Ceo * RT * SG * SA * SG * SC * SG * SG * SA | AAGGCTGAGCGGAGAAACCC | SOOOR SSSSS |
| 12525 | * RG * SA * SmA * SmA * SmC * SmC * SmC | | SSRSS SSSS |
| WV- | m5Ceo * SAeoAeoGeoGeo * RC * ST * SG * SA * SG * SC * SG * SG | CAAGGCTGAGCGGAGAAACC | SOOOR SSSSS |
| 12526 | * SA * RG * SmA * SmA * SmA * SmC * SmC | | SSSRS SSSS |
| WV- | m5Ceo * Sm5CeoAeoAeoGeo * RG * SC * ST * SG * SA * SG * | CCAAGGCTGAGCGGAGAAAC | SOOOR SSSSS |
| 12527 | SC * SG * SG * SA * RmG * SmA * SmA * SmA * SmC | | SSSSR SSSS |
| WV- | Teo * Sm5Ceom5CeoAeoAeo * RG * SG * SC * ST * SG * SA * | TCCAAGGCTGAGCGGAGAAA | SOOOR SSSSS |
| 12528 | SG * SC * SG * SG * SmA * SmG * SmA * SmA * SmA | | SSSSS SSSS |
| WV- | mG * SmA * SmG * SmC * SGeo * RG * SA * RG * SA * SA * | GAGCGGAGAAACCCTCCAAA | SSSSR SRSSS |
| 12529 | SA * SC * SC * SC * ST * Sm5Ceom5CeoAeoAeo * SAeo | | SSSSS OOOS |
| WV- | mU * SmG * SmA * SmG * Sm5Ceo * RG * SG * SA * RG * SA * | UGAGCGGAGAAACCCTCCAA | SSSSR SSRSS |
| 12530 | SA * SA * SC * SC * SC * STeom5Ceom5CeoAeo * SAeo | | SSSSS OOOS |
| WV- | mC * SmU * SmG * SmA * SGeo * RC * SG * SG * SA * RG * SA | CUGAGCGGAGAAACCCTCCA | SSSSR SSSRS |
| 12531 | * SA * SA * SC * SC * Sm5CeoTeom5Ceom5Ceo * SAeo | | SSSSS OOOS |
| WV- | mG * SmC * SmU * SmG * SAeo * RG * SC * SG * SG * SA * RG | GCUGAGCGGAGAAACCCTCC | SSSSR SSSSR |
| 12532 | * SA * SA * SA * SC * Sm5Ceom5CeoTeom5Ceo * Sm5Ceo | | SSSSS OOOS |
| WV- | mG * SmG * SmC * SmU * SGeo * RA * SG * SC * SG * SG * SA | GGCUGAGCGGAGAAACCCTC | SSSSR SSSSS |
| 12533 | * RG * SA * SA * SA * Sm5Ceom5Ceom5CeoTeo * Sm5Ceo | | RSSSS OOOS |
| WV- | mA * SmG * SmG * SmC * STeo * RG * SA * SG * SC * SG * SG | AGGCTGAGCGGAGAAACCCT | SSSSR SSSSS |
| 12534 | * SA * RG * SA * SA * SAeom5Ceom5Ceom5Ceo * STeo | | SRSSS OOOS |
| WV- | mA * SmA * SmG * SmG * Sm5Ceo * RT * SG * SA * SG * SC * | AAGGCTGAGCGGAGAAACCC | SSSSR SSSSS |
| 12535 | SG * SG * SA * RG * SA * SAeoAeom5Ceom5Ceo * Sm5Ceo | | SSRSS OOOS |
| WV- | mC * SmA * SmA * SmG * SGeo * RC * ST * SG * SA * SG * SC | CAAGGCTGAGCGGAGAAACC | SSSSR SSSSS |
| 12536 | * SG * SG * SA * RG * SAeoAeoAeom5Ceo * Sm5Ceo | | SSSRS OOOS |
| WV- | mC * SmC * SmA * SmA * SGeo * RG * SC * ST * SG * SA * SG | CCAAGGCTGAGCGGAGAAAC | SSSSR SSSSS |
| 12537 | * SC * SG * SG * SA * RGeoAeoAeoAeo * Sm5Ceo | | SSSSR OOOS |
| WV- | mU * SmC * SmC * SmA * SAeo * RG * SG * SC * ST * SG * SA | UCCAAGGCTGAGCGGAGAAA | SSSSR SSSSS |
| 12538 | * SG * SC * SG * SG * SAeoGeoAeoAeo * SAeo | | SSSSS OOOS |
| WV- | mG * STeon001Geon001m5Ceon001mA * SC * SA * SC * SA * | GTGCACACAGTAGATGAGGG | SnXnXnXS SSSSS |
| 12539 | SG * ST * SA * SG * RA * ST * SmGmAmGmG * SmG | | SSRSS OOOS |
| WV- | mG * STeoGeom5CeomA * SC * SA * SC * SA * SG * ST * SA * | GTGCACACAGTAGATGAGGG | SOOOS SSSSS |
| 12540 | SG * RA * ST * SmGn001mAn001mGn001mG * SmG | | SSRSS nXnXnXS |
| WV- | mG * STeon001Geon001m5Ceon001mA * SC * SA * SC * SA * | GTGCACACAGTAGATGAGGG | SnXnXnXS SSSSS |
| 12541 | SG * ST * SA * SG * RA * ST * SmGn001mAn001mGn001mG * | | SSRSS nXnXnXS |
| | SmG | | |
| WV- | mG * STeon001Geon001m5Ceon001mA * SC * SA * SC * SA * | GTGCACACAGTAGATGAGGG | SnXnXnXS SSSSS |
| 12542 | SG * ST * SA * SG * RA * ST * SmG * SmA * SmG * SmG * | | SSRSS SSSS |
| | SmG | | |
| WV- | mG * STeon001Geon001m5Ceon001mA * SC * SA * SC * SA * | GTGCACACAGTGGATGAGGG | SnXnXnXS SSSSS |
| 12543 | SG * ST * SG * SG * RA * ST * SmG * SmA * SmG * SmG * | | SSRSS SSSS |
| | SmG | | |
| WV- | mG * SGeon001m5Ceon001Aeon001mC * SA * SA * SG * SG * | GGCACAAGGGCGCAGACUUC | SnXnXnXS SSSSS |
| 12544 | SG * SC * SG * SC * RA * SG * SmA * SmC * SmU * SmU * | | SSRSS SSSS |
| | SmC | | |
| WV- | Aeo * Aeom5CeoTeom5Ceo * A * G * G * C * C * C * C * C * T * A | AACTCAGGCCCCCTACCCTG | XOOOX XXXXX |
| 13213 | * m5Ceom5Ceom5CeoTeo * Geo | | XXXXX OOOX |
| WV- | Geo * RAeo * RGeo * RGeo * RAeo * RG * SG * RC * SC * SG * ST | GAGGAGGCCGTGCAGGGCTC | RRRRR SRSSS |
| 13411 | * SG * SC * SA * SG * SGeo * RGeo * Rm5Ceo * RTeo * Rm5Ceo | | SSSSS RRRR |
| WV- | Aeo * RGeo * RAeo * RGeo * RGeo * RA * SG * SG * RC * SC * SG | AGAGGAGGCCGTGCAGGGCT | RRRRR SSRSS |
| 13412 | * ST * SG * SC * SA * SGeo * RGeo * RGeo * Rm5Ceo * RTeo | | SSSSS RRRR |
| WV- | Teo * RAeo * RGeo * RAeo * RGeo * RG * SA * SG * SG * RC * SC | TAGAGGAGGCCGTGCAGGGC | RRRRR SSSRS |
| 13413 | * SG * ST * SG * SC * SAeo * RGeo * RGeo * RGeo * Rm5Ceo | | SSSSS RRRR |
| WV- | Aeo * RTeo * RAeo * RGeo * RAeo * RG * SG * SA * SG * SG * RC | ATAGAGGAGGCCGTGCAGGG | RRRRR SSSSR |
| 13414 | * SC * SG * ST * SG * Sm5Ceo * RAeo * RGeo * RGeo * RGeo | | SSSSS RRRR |
| WV- | m5Ceo * RAeo * RTeo * RAeo * RGeo * RA * SG * SG * SA * SG * | CATAGAGGAGGCCGTGCAGG | RRRRR SSSSS |
| 13415 | SG * RC * SC * SG * ST * SGeo * Rm5Ceo * RAeo * RGeo * RGeo | | RSSSS RRRR |
| WV- | Aeo * Rm5Ceo * RAeo * RTeo * RAeo * RG * SA * SG * SG * SA * | ACATAGAGGAGGCCGTGCAG | RRRRR SSSSS |
| 13416 | SG * SG * RC * SC * SG * STeo * RGeo * Rm5Ceo * RAeo * RGeo | | SRSSS RRRR |
| WV- | m5Ceo * RAeo * Rm5Ceo * RAeo * RTeo * RA * SG * SA * SG * SG | CACATAGAGGAGGCCGTGCA | RRRRR SSSSS |
| 13417 | * SA * SG * SG * RC * SC * SGeo * RTeo * RGeo * Rm5Ceo * RAeo | | SSRSS RRRR |
| WV- | Geo * SAeoGeoGeoAeo * RG * SG * RC * SC * SG * ST * SG * SC * | GAGGAGGCCGTGCAGGGCTC | SOOOR SRSSS |
| 13418 | SA * SG * SGeoGeom5CeoTeo * Sm5Ceo | | SSSSS OOOS |
| WV- | Aeo * SGeoAeoGeoGeo * RA * SG * SG * RC * SC * SG * ST * SG * | AGAGGAGGCCGTGCAGGGCT | SOOOR SSRSS |
| 13419 | SC * SA * SGeoGeoGeom5Ceo * STeo | | SSSSS OOOS |
| WV- | Teo * SAeoGeoAeoGeo * RG * SA * SG * SG * RC * SC * SG * ST * | TAGAGGAGGCCGTGCAGGGC | SOOOR SSSRS |
| 13420 | SG * SC * SAeoGeoGeoGeo * Sm5Ceo | | SSSSS OOOS |
| WV- | Aeo * STeoAeoGeoAeo * RG * SG * SA * SG * SG * RC * SC * SG * | ATAGAGGAGGCCGTGCAGGG | SOOOR SSSSR |
| 13421 | ST * SG * Sm5CeoAeoGeoGeo * SGeo | | SSSSS OOOS |
| WV- | m5Ceo * SAeoTeoAeoGeo * RA * SG * SG * SA * SG * SG * RC * | CATAGAGGAGGCCGTGCAGG | SOOOR SSSSS |
| 13422 | SC * SG * ST * SGeom5CeoAeoGeo * SGeo | | RSSSS OOOS |
| WV- | Aeo * Sm5CeoAeoTeoAeo * RG * SA * SG * SG * SA * SG * SG * | ACATAGAGGAGGCCGTGCAG | SOOOR SSSSS |
| 13423 | RC * SC * SG * STeoGeom5CeoAeo * SGeo | | SRSSS OOOS |
| WV- | m5Ceo * SAeom5CeoAeoTeo * RA * SG * SA * SG * SG * SA * SG | CACATAGAGGAGGCCGTGCA | SOOOR SSSSS |
| 13424 | * SG * RC * SC * SGeoTeoGeom5Ceo * SAeo | | SSRSS OOOS |
| WV- | Geo * SAeoGeoGeoAeo * RG * SG * RC * SC * SG * ST * SG * SC * | GAGGAGGCCGTGCAGGGCUC | SOOOR SRSSS |
| 13425 | SA * SG * SmG * SmG * SmC * SmU * SmC | | SSSSS SSSS |
| WV- | Aeo * SGeoAeoGeoGeo * RA * SG * SG * RC * SC * SG * ST * SG * | AGAGGAGGCCGTGCAGGGCU | SOOOR SSRSS |
| 13426 | SC * SA * SmG * SmG * SmG * SmC * SmU | | SSSSS SSSS |
| WV- | Teo * SAeoGeoAeoGeo * RG * SA * SG * SG * RC * SC * SG * ST * | TAGAGGAGGCCGTGCAGGGC | SOOOR SSSRS |
| 13427 | SG * SC * SmA * SmG * SmG * SmG * SmC | | SSSSS SSSS |
| WV- | Aeo * STeoAeoGeoAeo * RG * SG * SA * SG * SG * RC * SC * SG * | ATAGAGGAGGCCGTGCAGGG | SOOOR SSSSR |
| 13428 | ST * SG * SmC * SmA * SmG * SmG * SmG | | SSSSS SSSS |
| WV- | m5Ceo * SAeoTeoAeoGeo * RA * SG * SG * SA * SG * SG * RC * | CATAGAGGAGGCCGTGCAGG | SOOOR SSSSS |
| 13429 | SC * SG * ST * SmG * SmC * SmA * SmG * SmG | | RSSSS SSSS |
| WV- | Aeo * Sm5CeoAeoTeoAeo * RG * SA * SG * SG * SA * SG * SG * | ACATAGAGGAGGCCGUGCAG | SOOOR SSSSS |
| 13430 | RC * SC * SG * SmU * SmG * SmC * SmA * SmG | | SRSSS SSSS |
| WV- | m5Ceo * SAeom5CeoAeoTeo * RA * SG * SA * SG * SG * SA * SG | CACATAGAGGAGGCCGUGCA | SOOOR SSSSS |
| 13431 | * SG * RC * SC * SmG * SmU * SmG * SmC * SmA | | SSRSS SSSS |
| WV- | mG * SmA * SmG * SmG * SmA * SG * SG * RC * SC * SG * ST * | GAGGAGGCCGTGCAGGGCTC | SSSSS SRSSS |
| 13432 | SG * SC * SA * SG * SGeoGeom5CeoTeo * Sm5Ceo | | SSSSS OOOS |
| WV- | mA * SmG * SmA * SmG * SmG * SA * SG * SG * RC * SC * SG * | AGAGGAGGCCGTGCAGGGCT | SSSSS SSRSS |
| 13433 | ST * SG * SC * SA * SGeoGeoGeom5Ceo * STeo | | SSSSS OOOS |
| WV- | mU * SmA * SmG * SmA * SmG * SG * SA * SG * SG * RC * SC * | UAGAGGAGGCCGTGCAGGGC | SSSSS SSSRS |
| 13434 | SG * ST * SG * SC * SAeoGeoGeoGeo * Sm5Ceo | | SSSSS OOOS |
| WV- | mA * SmU * SmA * SmG * SmA * SG * SG * SA * SG * SG * RC * | AUAGAGGAGGCCGTGCAGGG | SSSSS SSSSR |
| 13435 | SC * SG * ST * SG * Sm5CeoAeoGeoGeo * SGeo | | SSSSS OOOS |
| WV- | mC * SmA * SmU * SmA * SmG * SA * SG * SG * SA * SG * SG * | CAUAGAGGAGGCCGTGCAGG | SSSSS SSSSS |
| 13436 | RC * SC * SG * ST * SGeom5CeoAeoGeo * SGeo | | RSSSS OOOS |
| WV- | mA * SmC * SmA * SmU * SmA * SG * SA * SG * SG * SA * SG * | ACAUAGAGGAGGCCGTGCAG | SSSSS SSSSS |
| 13437 | SG * RC * SC * SG * STeoGeom5CeoAeo * SGeo | | SRSSS OOOS |
| WV- | mC * SmA * SmC * SmA * SmU * SA * SG * SA * SG * SG * SA * | CACAUAGAGGAGGCCGTGCA | SSSSS SSSSS |
| 13438 | SG * SG * RC * SC * SGeoTeoGeom5Ceo * SAeo | | SSRSS OOOS |
| WV- | 1G * Geo * m5Ceo * Aeo * m5Ceo * Aeo * Aeo * G * G * G * C * G * | GGCACAAGGGCGCAGACTTC | XXXXX XXXXX |
| 13625 | C * A * G * A * C * Teo * Teo * m5Ceo * m5Ceo * Aeo * Aeo * 1A | CAAA | XXXXX XXXXX |
| | | | XXX |
| WV- | m51C * Aeo * m5Ceo * Aeo * 1A * G * G * G * C * G * C * A * G * A | CACAAGGGCGCAGACTTCCA | XXXXX XXXXX |
| 13626 | * C * 1T * Teo * m5Ceo * m5Ceo * 1A | | XXXXX XXXX |
| WV- | m51C * Aeo * m5Ceo * 1A * Aeo * G * G * G * C * G * C * A * G * A | CACAAGGGCGCAGACTTCCA | XXXXX XXXXX |
| 13627 | * C * Teo * 1T * m5Ceo * m5Ceo * 1A | | XXXXX XXXX |
| WV- | m51C * Aeo * m51C * Aeo * Aeo * G * G * G * C * G * C * A * G * A | CACAAGGGCGCAGACTTCCA | XXXXX XXXXX |
| 13628 | * C * Teo * Teo * m51C * m5Ceo * 1A | | XXXXX XXXX |
| WV- | m51C * 1A * m5Ceo * Aeo * Aeo * G * G * G * C * G * C * A * G * A | CACAAGGGCGCAGACTTCCA | XXXXX XXXXX |
| 13629 | * C * Teo * Teo * m5Ceo * m51C * 1A | | XXXXX XXXX |
| WV- | m51C * 1A * m5Ceo * Aeo * Aeo * G * G * G * C * G * C * A * G * A | CACAAGGGCGCAGACTTCCA | XXXXX XXXXX |
| 13630 | * C * Teo * Teo * m5Ceo * m5Ceo * Aeo | | XXXXX XXXX |
| WV- | m5Ceo * Aeo * m5Ceo * Aeo * Aeo * G * G * G * C * G * C * A * G | CACAAGGGCGCAGACTTCCA | XXXXX XXXXX |
| 13631 | * A * C * Teo * Teo * m5Ceo * m51C * 1A | | XXXXX XXXX |
| WV- | m51C * A * m5Ceo * Aeo * Aeo * Geo * G * G * C * G * C * A * G * | CACAAGGGCGCAGACTTCCA | XXXXX XXXXX |
| 13632 | A * m5Ceo * Teo * Teo * m5Ceo * C * 1A | | XXXXX XXXX |
| WV- | m51C * Aeo * C * Aeo * Aeo * Geo * G * G * C * G * C * A * G * A | CACAAGGGCGCAGACTTCCA | XXXXX XXXXX |
| 13633 | * m5Ceo * Teo * Teo * C * m5Ceo * 1A | | XXXXX XXXX |
| WV- | m51C * Aeo * m5Ceo * A * Aeo * Geo * G * G * C * G * C * A * G * | CACAAGGGCGCAGACTTCCA | XXXXX XXXXX |
| 13634 | A * m5Ceo * Teo * T * m5Ceo * m5Ceo * 1A | | XXXXX XXXX |
| WV- | m51C * Aeo * m5Ceo * Aeo * A * Geo * G * G * C * G * C * A * G * | CACAAGGGCGCAGACTTCCA | XXXXX XXXXX |
| 13635 | A * m5Ceo * T * Teo * m5Ceo * m5Ceo * 1A | | XXXXX XXXX |
| WV- | m51C * Aeo * m5Ceo * Aeo * 1A * G * SG * SG * SC * SG * SC * RA | CACAAGGGCGCAGACTTCCA | XXXXX SSSSS |
| 13636 | * SG * SA * SC * 1T * Teo * m5Ceo * m5Ceo * 1A | | RSSSX XXXX |
| WV- | m51C * Aeo * m5Ceo * 1A * Aeo * G * SG * SG * SC * SG * SC * RA | CACAAGGGCGCAGACTTCCA | XXXXX SSSSS |
| 13637 | * SG * SA * SC * Teo * 1T * m5Ceo * m5Ceo * 1A | | RSSSX XXXX |
| WV- | m51C * Aeo * m51C * Aeo * Aeo * G * SG * SG * SC * SG * SC * RA | CACAAGGGCGCAGACTTCCA | XXXXX SSSSS |
| 13638 | * SG * SA * SC * Teo * Teo * m51C * m5Ceo * 1A | | RSSSX XXXX |
| WV- | m51C * 1A * m5Ceo * Aeo * Aeo * G * SG * SG * SC * SG * SC * RA | CACAAGGGCGCAGACTTCCA | XXXXX SSSSS |
| 13639 | * SG * SA * SC * Teo * Teo * m5Ceo * m51C * 1A | | RSSSX XXXX |
| WV- | m51C * 1A * m5Ceo * Aeo * Aeo * G * SG * SG * SC * SG * SC * RA | CACAAGGGCGCAGACTTCCA | XXXXX SSSSS |
| 13640 | * SG * SA * SC * Teo * Teo * m5Ceo * m5Ceo * Aeo | | RSSSX XXXX |
| WV- | m5Ceo * Aeo * m5Ceo * Aeo * Aeo * G * SG * SG * SC * SG * SC * | CACAAGGGCGCAGACTTCCA | XXXXX SSSSS |
| 13641 | RA * SG * SA * SC * Teo * Teo * m5Ceo * m51C * 1A | | RSSSX XXXX |
| WV- | m51C * A * m5Ceo * Aeo * Aeo * Geo * G * SG * SC * SG * SC * | CACAAGGGCGCAGACTTCCA | XXXXX XSSSS |
| 13642 | RA * SG * SA * m5Ceo * Teo * Teo * m5Ceo * C * 1A | | RSSXX XXXX |
| WV- | m51C * Aeo * C * Aeo * Aeo * Geo * G * SG * SC * SG * SC * RA * | CACAAGGGCGCAGACTTCCA | XXXXX XSSSS |
| 13643 | SG * SA * m5Ceo * Teo * Teo * C * m5Ceo * 1A | | RSSXX XXXX |
| WV- | m51C * Aeo * m5Ceo * A * Aeo * Geo * G * SG * SC * SG * SC * | CACAAGGGCGCAGACTTCCA | XXXXX XSSSS |
| 13644 | RA * SG * SA * m5Ceo * Teo * T * m5Ceo * m5Ceo * 1A | | RSSXX XXXX |
| WV- | m51C * Aeo * m5Ceo * Aeo * A * Geo * G * SG * SC * SG * SC * | CACAAGGGCGCAGACTTCCA | XXXXX XSSSS |
| 13645 | RA * SG * SA * m5Ceo * T * Teo * m5Ceo * m5Ceo * 1A | | RSSXX XXXX |
| WV- | m51C * Aeo * m5Ceo * Aeo * Aeo * G * G * G * C * A * C * A * G * | CACAAGGGCACAGACTTCCA | XXXXX XXXXX |
| 13646 | A * C * Teo * Teo * m5Ceo * m5Ceo * 1A | | XXXXX XXXX |
| WV- | m51C * Aeo * m5Ceo * Aeo *1A * G * G * G * C * A * C * A * G * A | CACAAGGGCACAGACTTCCA | XXXXX XXXXX |
| 13647 | * C * 1T * Teo * m5Ceo * m5Ceo * 1A | | XXXXX XXXX |
| WV- | m51C * Aeo * m5Ceo * 1A * Aeo * G * G * G * C * A * C * A * G * A | CACAAGGGCACAGACTTCCA | XXXXX XXXXX |
| 13648 | * C * Teo *1T * m5Ceo * m5Ceo * 1A | | XXXXX XXXX |
| WV- | m51C * Aeo * m51C * Aeo * Aeo * G * G * G * C * A * C * A * G * A | CACAAGGGCACAGACTTCCA | XXXXX XXXXX |
| 13649 | * C * Teo * Teo * m51C * m5Ceo * 1A | | XXXXX XXXX |
| WV- | m51C *1A * m5Ceo * Aeo * Aeo * G * G * G * C * A * C * A * G * A | CACAAGGGCACAGACTTCCA | XXXXX XXXXX |
| 13650 | * C * Teo * Teo * m5Ceo * m51C * 1A | | XXXXX XXXX |
| WV- | m51C *1A * m5Ceo * Aeo * Aeo * G * G * G * C * A * C * A * G * A | CACAAGGGCACAGACTTCCA | XXXXX XXXXX |
| 13651 | * C * Teo * Teo * m5Ceo * m5Ceo * Aeo | | XXXXX XXXX |
| WV- | m5Ceo * Aeo * m5Ceo * Aeo * Aeo * G * G * G * C * A * C * A * G | CACAAGGGCACAGACTTCCA | XXXXX XXXXX |
| 13652 | * A * C * Teo * Teo * m5Ceo * m51C * 1A | | XXXXX XXXX |
| WV- | m51C * A * m5Ceo * Aeo * Aeo * Geo * G * G * C * A * C * A * G * | CACAAGGGCACAGACTTCCA | XXXXX XXXXX |
| 13653 | A * m5Ceo * Teo * Teo * m5Ceo * C * 1A | | XXXXX XXXX |
| WV- | m51C * Aeo * C * Aeo * Aeo * Geo * G * G * C * A * C * A * G * A | CACAAGGGCACAGACTTCCA | XXXXX XXXXX |
| 13654 | * m5Ceo * Teo * Teo * C * m5Ceo * 1A | | XXXXX XXXX |
| WV- | m51C * Aeo * m5Ceo * A * Aeo * Geo * G * G * C * A * C * A * G * | CACAAGGGCACAGACTTCCA | XXXXX XXXXX |
| 13655 | A * m5Ceo * Teo * T * m5Ceo * m5Ceo * 1A | | XXXXX XXXX |
| WV- | m51C * Aeo * m5Ceo * Aeo * A * Geo * G * G * C * A * C * A * G * | CACAAGGGCACAGACTTCCA | XXXXX XXXXX |
| 13656 | A * m5Ceo * T * Teo * m5Ceo * m5Ceo * 1A | | XXXXX XXXX |
| WV- | m51C * Aeo * m5Ceo * Aeo * 1A * G * SG * SG * SC * SA * SC * RA | CACAAGGGCACAGACTTCCA | XXXXX SSSSS |
| 13657 | * SG * SA * SC * 1T * Teo * m5Ceo * m5Ceo * 1A | | RSSSX XXXX |
| WV- | m51C * Aeo * m5Ceo * 1A * Aeo * G * SG * SG * SC * SA * SC * RA | CACAAGGGCACAGACTTCCA | XXXXX SSSSS |
| 13658 | * SG * SA * SC * Teo * 1T * m5Ceo * m5Ceo * 1A | | RSSSX XXXX |
| WV- | m51C * Aeo * m51C * Aeo * Aeo * G * SG * SG * SC * SA * SC * RA | CACAAGGGCACAGACTTCCA | XXXXX SSSSS |
| 13659 | * SG * SA * SC * Teo * Teo * m51C * m5Ceo * 1A | | RSSSX XXXX |
| WV- | m51C * 1A * m5Ceo * Aeo * Aeo * G * SG * SG * SC * SA * SC * RA | CACAAGGGCACAGACTTCCA | XXXXX SSSSS |
| 13660 | * SG * SA * SC * Teo * Teo * m5Ceo * m51C * 1A | | RSSSX XXXX |
| WV- | m51C * 1A * m5Ceo * Aeo * Aeo * G * SG * SG * SC * SA * SC * RA | CACAAGGGCACAGACTTCCA | XXXXX SSSSS |
| 13661 | * SG * SA * SC * Teo * Teo * m5Ceo * m5Ceo * Aeo | | RSSSX XXXX |
| WV- | m5Ceo * Aeo * m5Ceo * Aeo * Aeo * G * SG * SG * SC * SA * SC * | CACAAGGGCACAGACTTCCA | XXXXX SSSSS |
| 13662 | RA * SG * SA * SC * Teo * Teo * m5Ceo * m51C * 1A | | RSSSX XXXX |
| WV- | m51C * A * m5Ceo * Aeo * Aeo * Geo * G * SG * SC * SA * SC * | CACAAGGGCACAGACTTCCA | XXXXX XSSSS |
| 13663 | RA * SG * SA * m5Ceo * Teo * Teo * m5Ceo * C * 1A | | RSSXX XXXX |
| WV- | m51C * Aeo * C * Aeo * Aeo * Geo * G * SG * SC * SA * SC * RA * | CACAAGGGCACAGACTTCCA | XXXXX XSSSS |
| 13664 | SG * SA * m5Ceo * Teo * Teo * C * m5Ceo * 1A | | RSSXX XXXX |
| WV- | m51C * Aeo * m5Ceo * A * Aeo * Geo * G * SG * SC * SA * SC * | CACAAGGGCACAGACTTCCA | XXXXX XSSSS |
| 13665 | RA * SG * SA * m5Ceo * Teo * T * m5Ceo * m5Ceo * 1A | | RSSXX XXXX |
| WV- | m51C * Aeo * m5Ceo * Aeo * A * Geo * G * SG * SC * SA * SC * | CACAAGGGCACAGACTTCCA | XXXXX XSSSS |
| 13666 | RA * SG * SA * m5Ceo * T * Teo * m5Ceo * m5Ceo * 1A | | RSSXX XXXX |
| WV- | Geo * Geo * m5Ceo * Aeo * m5Ceo * Aeo * Aeo * G * G * G * C * G | GGCACAAGGGCGCAGACTTC | XXXXX XXXXX |
| 13667 | * C * A * G * A * C * Teo * Teo * m5Ceo * m5Ceo * Aeo * Aeo * | CAAA | XXXXX XXXXX |
| | Aeo | | XXX |
| WV- | mG * STeoTeoAeomC * RC * RG * SC * SC * SA * ST * SC * SC * | GTTACCGCCATCCCCGCCGU | SOOOR RSSSS |
| 13880 | SC * SC * RmG * SmC * SmC * SmG * SmU | | SSSSR SSSS |
| WV- | mG * STeoTeoAeomC * RC * SG * RC * SC * SA * ST * SC * SC * | GTTACCGCCATCCCCGCCGU | SOOOR SRSSS |
| 13881 | SC * SC * RmG * SmC * SmC * SmG * SmU | | SSSSR SSSS |
| WV- | mG * STeoTeoAeomC * RC * SG * SC * RC * SA * ST * SC * SC * | GTTACCGCCATCCCCGCCGU | SOOOR SSRSS |
| 13882 | SC * SC * RmG * SmC * SmC * SmG * SmU | | SSSSR SSSS |
| WV- | mG * STeoTeoAeomC * RC * SG * SC * SC * RA * ST * SC * SC * | GTTACCGCCATCCCCGCCGU | SOOOR SSSRS |
| 13883 | SC * SC * RmG * SmC * SmC * SmG * SmU | | SSSSR SSSS |
| WV- | mG * STeoTeoAeomC * RC * SG * SC * SC * SA * RT * SC * SC * | GTTACCGCCATCCCCGCCGU | SOOOR SSSSR |
| 13884 | SC * SC * RmG * SmC * SmC * SmG * SmU | | SSSSR SSSS |
| WV- | mG * STeoTeoAeomC * RC * SG * SC * SC * SA * ST * RC * SC * | GTTACCGCCATCCCCGCCGU | SOOOR SSSSS |
| 13885 | SC * SC * RmG * SmC * SmC * SmG * SmU | | RSSSR SSSS |
| WV- | mG * STeoTeoAeomC * RC * SG * SC * SC * SA * ST * SC * RC * | GTTACCGCCATCCCCGCCGU | SOOOR SSSSS |
| 13886 | SC * SC * RmG * SmC * SmC * SmG * SmU | | SRSSR SSSS |
| WV- | mG * STeoTeoAeomC * RC * SG * SC * SC * SA * ST * SC * SC * | GTTACCGCCATCCCCGCCGU | SOOOR SSSSS |
| 13887 | RC * SC * RmG * SmC * SmC * SmG * SmU | | SSRSR SSSS |
| WV- | mG * STeoTeoAeomC * RC * SG * SC * SC * SA * ST * SC * SC * | GTTACCGCCATCCCCGCCGU | SOOOR SSSSS |
| 13888 | SC * RC * RmG * SmC * SmC * SmG * SmU | | SSSRR SSSS |
| WV- | mG * STeoTeoAeomC * RC * SG * SC * SC * SA * ST * SC * SC * | GTTACCGCCATCCCCGCCGU | SOOOR SSSSS |
| 13889 | SC * SC * RmG * SmC * SmC * SmG * SmU | | SSSSR SSSS |
| WV- | Geo * STeoTeoAeom5Ceo * RC * RG * SC * SC * SA * ST * SC * | GTTACCGCCATCCCCGCCGT | SOOOR RSSSS |
| 13890 | SC * SC * SC * RGeom5Ceom5CeoGeo * STeo | | SSSSR OOOS |
| WV- | Geo * STeoTeoAeom5Ceo * RC * SG * RC * SC * SA * ST * SC * | GTTACCGCCATCCCCGCCGT | SOOOR SRSSS |
| 13891 | SC * SC * SC * RGeom5Ceom5CeoGeo * STeo | | SSSSR OOOS |
| WV- | Geo * STeoTeoAeom5Ceo * RC * SG * SC * RC * SA * ST * SC * | GTTACCGCCATCCCCGCCGT | SOOOR SSRSS |
| 13892 | SC * SC * SC * RGeom5Ceom5CeoGeo * STeo | | SSSSR OOOS |
| WV- | Geo * STeoTeoAeom5Ceo * RC * SG * SC * SC * RA * ST * SC * | GTTACCGCCATCCCCGCCGT | SOOOR SSSRS |
| 13893 | SC * SC * SC * RGeom5Ceom5CeoGeo * STeo | | SSSSR OOOS |
| WV- | Geo * STeoTeoAeom5Ceo * RC * SG * SC * SC * SA * RT * SC * | GTTACCGCCATCCCCGCCGT | SOOOR SSSSR |
| 13894 | SC * SC * SC * RGeom5Ceom5CeoGeo * STeo | | SSSSR OOOS |
| WV- | Geo * STeoTeoAeom5Ceo * RC * SG * SC * SC * SA * ST * RC * | GTTACCGCCATCCCCGCCGT | SOOOR SSSSS |
| 13895 | SC * SC * SC * RGeom5Ceom5CeoGeo * STeo | | RSSSR OOOS |
| WV- | Geo * STeoTeoAeom5Ceo * RC * SG * SC * SC * SA * ST * SC * | GTTACCGCCATCCCCGCCGT | SOOOR SSSSS |
| 13896 | RC * SC * SC * RGeom5Ceom5CeoGeo * STeo | | SRSSR OOOS |
| WV- | Geo * STeoTeoAeom5Ceo * RC * SG * SC * SC * SA * ST * SC * SC | GTTACCGCCATCCCCGCCGT | SOOOR SSSSS |
| 13897 | * RC * SC * RGeom5Ceom5CeoGeo * STeo | | SSRSR OOOS |
| WV- | Geo * STeoTeoAeom5Ceo * RC * SG * SC * SC * SA * ST * SC * SC | GTTACCGCCATCCCCGCCGT | SOOOR SSSSS |
| 13898 | * SC * RC * RGeom5Ceom5CeoGeo * STeo | | SSSRR OOOS |
| WV- | Geo * STeoTeoAeom5Ceo * RC * SG * SC * SC * SA * ST * SC * SC | GTTACCGCCATCCCCGCCGT | SOOOR SSSSS |
| 13899 | * SC * SC * RGeom5Ceom5CeoGeo * STeo | | SSSSR OOOS |
| WV- | mA * Sm5Ceom5CeoGeomC * RC * RA * ST * SC * SC * SC * SC * | ACCGCCATCCCCGCCGUAGC | SOOOR RSSSS |
| 13900 | SG * SC * SC * RmG * SmU * SmA * SmG * SmC | | SSSSR SSSS |
| WV- | mA * Sm5Ceom5CeoGeomC * RC * SA * RT * SC * SC * SC * SC * | ACCGCCATCCCCGCCGUAGC | SOOOR SRSSS |
| 13901 | SG * SC * SC * RmG * SmU * SmA * SmG * SmC | | SSSSR SSSS |
| WV- | mA * Sm5Ceom5CeoGeomC * RC * SA * ST * RC * SC * SC * SC * | ACCGCCATCCCCGCCGUAGC | SOOOR SSRSS |
| 13902 | SG * SC * SC * RmG * SmU * SmA * SmG * SmC | | SSSSR SSSS |
| WV- | mA * Sm5Ceom5CeoGeomC * RC * SA * ST * SC * RC * SC * SC * | ACCGCCATCCCCGCCGUAGC | SOOOR SSSRS |
| 13903 | SG * SC * SC * RmG * SmU * SmA * SmG * SmC | | SSSSR SSSS |
| WV- | mA * Sm5Ceom5CeoGeomC * RC * SA * ST * SC * SC * RC * SC * | ACCGCCATCCCCGCCGUAGC | SOOOR SSSSR |
| 13904 | SG * SC * SC * RmG * SmU * SmA * SmG * SmC | | SSSSR SSSS |
| WV- | mA * Sm5Ceom5CeoGeomC * RC * SA * ST * SC * SC * SC * RC * | ACCGCCATCCCCGCCGUAGC | SOOOR SSSSS |
| 13905 | SG * SC * SC * RmG * SmU * SmA * SmG * SmC | | RSSSR SSSS |
| WV- | mA * Sm5Ceom5CeoGeomC * RC * SA * ST * SC * SC * SC * SC * | ACCGCCATCCCCGCCGUAGC | SOOOR SSSSS |
| 13906 | RG * SC * SC * RmG * SmU * SmA * SmG * SmC | | SRSSR SSSS |
| WV- | mA * Sm5Ceom5CeoGeomC * RC * SA * ST * SC * SC * SC * SC * | ACCGCCATCCCCGCCGUAGC | SOOOR SSSSS |
| 13907 | SG * RC * SC * RmG * SmU * SmA * SmG * SmC | | SSRSR SSSS |
| WV- | mA * Sm5Ceom5CeoGeomC * RC * SA * ST * SC * SC * SC * SC * | ACCGCCATCCCCGCCGUAGC | SOOOR SSSSS |
| 13908 | SG * SC * RC * RmG * SmU * SmA * SmG * SmC | | SSSRR SSSS |
| WV- | mA * Sm5Ceom5CeoGeomC * RC * SA * ST * SC * SC * SC * SC * | ACCGCCATCCCCGCCGUAGC | SOOOR SSSSS |
| 13909 | SG * SC * SC * RmG * SmU * SmA * SmG * SmC | | SSSSR SSSS |
| WV- | Aeo * Sm5Ceom5CeoGeom5Ceo * RC * RA * ST * SC * SC * SC * SC | ACCGCCATCCCCGCCGTAGC | SOOOR RSSSS |
| 13910 | * SG * SC * SC * RGeoTeoAeoGeo * Sm5Ceo | | SSSSR OOOS |
| WV- | Aeo * Sm5Ceom5CeoGeom5Ceo * RC * SA * RT * SC * SC * SC * SC | ACCGCCATCCCCGCCGTAGC | SOOOR SRSSS |
| 13911 | * SG * SC * SC * RGeoTeoAeoGeo * Sm5Ceo | | SSSSR OOOS |
| WV- | Aeo * Sm5Ceom5CeoGeom5Ce * RC * SA * ST * RC * SC * SC * SC | ACCGCCATCCCCGCCGTAGC | SOOOR SSRSS |
| 13912 | * SG * SC * SC * RGeoTeoAeoGeo * Sm5Ceo | | SSSSR OOOS |
| WV- | Aeo * Sm5Ceom5CeoGeom5Ceo * RC * SA * ST * SC * RC * SC * SC | ACCGCCATCCCCGCCGTAGC | SOOOR SSSRS |
| 13913 | * SG * SC * SC * RGeoTeoAeoGeo * Sm5Ceo | | SSSSR OOOS |
| WV- | Aeo * Sm5Ceom5CeoGeom5Ceo * RC * SA * ST * SC * SC * RC * SC | ACCGCCATCCCCGCCGTAGC | SOOOR SSSSR |
| 13914 | * SG * SC * SC * RGeoTeoAeoGeo * Sm5Ceo | | SSSSR OOOS |
| WV- | Aeo * Sm5Ceom5CeoGeom5Ceo * RC * SA * ST * SC * SC * SC * RC | ACCGCCATCCCCGCCGTAGC | SOOOR SSSSS |
| 13915 | * SG * SC * SC * RGeoTeoAeoGeo * Sm5Ceo | | RSSSR OOOS |
| WV- | Aeo * Sm5Ceom5CeoGeom5Ceo * RC * SA * ST * SC * SC * SC * SC | ACCGCCATCCCCGCCGTAGC | SOOOR SSSSS |
| 13916 | * RG * SC * SC * RGeoTeoAeoGeo * Sm5Ceo | | SRSSR OOOS |
| WV- | Aeo * Sm5Ceom5CeoGeom5Ceo * RC * SA * ST * SC * SC * SC * SC | ACCGCCATCCCCGCCGTAGC | SOOOR SSSSS |
| 13917 | * SG * RC * SC * RGeoTeoAeoGeo * Sm5Ceo | | SSRSR OOOS |
| WV- | Aeo * Sm5Ceom5CeoGeom5Ceo * RC * SA * ST * SC * SC * SC * SC | ACCGCCATCCCCGCCGTAGC | SOOOR SSSSS |
| 13918 | * SG * SC * RC * RGeoTeoAeoGeo * Sm5Ceo | | SSSRR OOOS |
| WV- | Aeo * Sm5Ceom5CeoGeom5Ceo * RC * SA * ST * SC * SC * SC * SC | ACCGCCATCCCCGCCGTAGC | SOOOR SSSSS |
| 13919 | * SG * SC * SC * RGeoTeoAeoGeo * Sm5Ceo | | SSSSR OOOS |
| WV- | m5Ceo * RAeoAeoGeoGeo * RG * SC * SG * SC * RA * SG * SA * SC | CAAGGGCGCAGACTTCCAAA | ROOOR SSSRS |
| 13920 | * ST * ST * Rm5Ceo * SmC * SmA * SmA * SmA | | SSSSR SSSS |
| WV- | Aeo * Rm5CeoAeoAeoGeo * RG * SG * SC * SG * SC * RA * SG * SA | ACAAGGGCGCAGACTTCCAA | ROOOR SSSSR |
| 13921 | * SC * ST * RTeo * SmC * SmC * SmA * SmA | | SSSSR SSSS |
| WV- | m5Ceo * RAeom5CeoAeoAeo * RG * SG * SG * SC * SG * SC * RA * | CACAAGGGCGCAGACTUCCA | ROOOR SSSSS |
| 13922 | SG * SA * SC * RTeo * SmU * SmC * SmC * SmA | | RSSSR SSSS |
| WV- | Geo * Rm5CeoAeom5CeoAeo * RA * SG * SG * SG * SC * SG * SC * | GCACAAGGGCGCAGACUUCC | ROOOR SSSSS |
| 13923 | RA * SG * SA * Rm5Ceo * SmU * SmU * SmC * SmC | | SRSSR SSSS |
| WV- | Geo * RGeom5CeoAeom5Ceo * RA * SA * SG * SG * SG * SC * SG * | GGCACAAGGGCGCAGACUUC | ROOOR SSSSS |
| 13924 | SC * RA * SG * RAeo * SmC * SmU * SmU * SmC | | SSRSR SSSS |
| WV- | m5Ceo * RAeoAeoGeoGeo * RG * SC * SG * SC * RA * SG * SA * SC | CAAGGGCGCAGACTTCCAAA | ROOOR SSSRS |
| 13925 | * ST * ST * Rm5Ceom5CeoAeoAeo * RAeo | | SSSSR OOOR |
| WV- | Aeo * Rm5CeoAeoAeoGeo * RG * SG * SC * SG * SC * RA * SG * SA | ACAAGGGCGCAGACTTCCAA | ROOOR SSSSR |
| 13926 | * SC * ST * RTeom5Ceom5CeoAeo * RAeo | | SSSSR OOOR |
| WV- | m5Ceo * RAeom5CeoAeoAeo * RG * SG * SG * SC * SG * SC * RA * | CACAAGGGCGCAGACTTCCA | ROOOR SSSSS |
| 13927 | SG * SA * SC * RTeoTeom5Ceom5Ceo * RAeo | | RSSSR OOOR |
| WV- | Geo * Rm5CeoAeom5CeoAeo * RA * SG * SG * SG * SC * SG * SC * | GCACAAGGGCGCAGACTTCC | ROOOR SSSSS |
| 13928 | RA * SG * SA * Rm5CeoTeoTeom5Ceo * Rm5Ceo | | SRSSR OOOR |
| WV- | Geo * RGeom5CeoAeom5Ceo * RA * SA * SG * SG * SG * SC * SG * | GGCACAAGGGCGCAGACTTC | ROOOR SSSSS |
| 13929 | SC * RA * SG * RAeom5CeoTeoTeo * Rm5Ceo | | SSRSR OOOR |
| WV- | m5Ceo * RAeo * RAeo * RGeo * RGeo * RG * SC * SG * SC * RA * | CAAGGGCGCAGACTTCCAAA | RRRRR SSSRS |
| 13930 | SG * SA * SC * ST * ST * Rm5Ceo * Rm5Ceo * RAeo * RAeo * RAeo | | SSSSR RRRR |
| WV- | Aeo * Rm5Ceo * RAeo * RTeo * RAeo * RG * SA * SG * SG * SA * | ACATAGAGGAGGCCGTGCAG | RRRRR SSSSS |
| 13931 | SG * SG * RC * SC * SG * Teo * RGeo * Rm5Ceo * RAeo * RGeo | | SRSSX RRRR |
| WV- | m5Ceo * RAeo * Rm5Ceo * RAeo * RAeo * RG * SG * SG * SC * SG * | CACAAGGGCGCAGACTTCCA | RRRRR SSSSS |
| 13932 | SC * RA * SG * SA * SC * RTeo * RTeo * Rm5Ceo * Rm5Ceo * RAeo | | RSSSR RRRR |
| WV- | Geo * Rm5Ceo * RAeo * Rm5Ceo * RAeo * RA * SG * SG * SG * SC * | GCACAAGGGCGCAGACTTCC | RRRRR SSSSS |
| 13933 | SG * SC * RA * SG * SA * Rm5Ceo * RTeo * RTeo * Rm5Ceo * | | SRSSR RRRR |
| | Rm5Ceo | | |
| WV- | Geo * RGeo * Rm5Ceo * RAeo * Rm5Ceo * RA * SA * SG * SG * SG * | GGCACAAGGGCGCAGACTTC | RRRRR SSSSS |
| 13934 | SC * SG * SC * RA * SG * RAeo * Rm5Ceo * RTeo * RTeo * Rm5Ceo | | SSRSR RRRR |
| WV- | m5Ceo * RAeoAeoGeoGeo * RG * SC * SA * SC * RA * SG * SA * SC | CAAGGGCACAGACTTCCAAA | ROOOR SSSRS |
| 13935 | * ST * ST * Rm5Ceo * SmC * SmA * SmA * SmA | | SSSSR SSSS |
| WV- | Aeo * Rm5CeoAeoAeoGeo * RG * SG * SC * SA * SC * RA * SG * SA | ACAAGGGCACAGACTTCCAA | ROOOR SSSSR |
| 13936 | * SC * ST * RTeo * SmC * SmC * SmA * SmA | | SSSSR SSSS |
| WV- | m5Ceo * RAeom5CeoAeoAeo * RG * SG * SG * SC * SA * SC * RA * | CACAAGGGCACAGACTUCCA | ROOOR SSSSS |
| 13937 | SG * SA * SC * RTeo * SmU * SmC * SmC * SmA | | RSSSR SSSS |
| WV- | Geo * Rm5CeoAeom5CeoAeo * RA * SG * SG * SG * SC * SA * SC * | GCACAAGGGCA CAGACUUCC | ROOOR SSSSS |
| 13938 | RA * SG * SA * Rm5Ceo * SmU * SmU * SmC * SmC | | SRSSR SSSS |
| WV- | Geo * RGeom5CeoAeom5Ceo * RA * SA * SG * SG * SG * SC * SA * | GGCACAAGGGCA CAGACUUC | ROOOR SSSSS |
| 13939 | SC * RA * SG * RAeo * SmC * SmU * SmU * SmC | | SSRSR SSSS |
| WV- | m5Ceo * RAeoAeoGeoGeo * RG * SC * SA * SC * RA * SG * SA * SC | CAAGGGCACAGACTTCCAAA | ROOOR SSSRS |
| 13940 | * ST * ST * Rm5Ceom5CeoAeoAeo * RAeo | | SSSSR OOOR |
| WV- | Aeo * Rm5CeoAeoAeoGeo * RG * SG * SC * SA * SC * RA * SG * SA | ACAAGGGCACAGACTTCCAA | ROOOR SSSSR |
| 13941 | * SC * ST * RTeom5Ceom5CeoAeo * RAeo | | SSSSR OOOR |
| WV- | m5Ceo * RAeom5CeoAeoAeo * RG * SG * SG * SC * SA * SC * RA * | CACAAGGGCACAGACTTCCA | ROOOR SSSSS |
| 13942 | SG * SA * SC * RTeoTeom5Ceom5Ceo * RAeo | | RSSSR OOOR |
| WV- | Geo * Rm5CeoAeom5CeoAeo * RA * SG * SG * SG * SC * SA * SC * | GCACAAGGGCA CAGACTTCC | ROOOR SSSSS |
| 13943 | RA * SG * SA * Rm5CeoTeoTeom5Ceo * Rm5Ceo | | SRSSR OOOR |
| WV- | Geo * RGeom5CeoAeom5Ceo * RA * SA * SG * SG * SG * SC * SA * | GGCACAAGGGCA CAGACTTC | ROOOR SSSSS |
| 13944 | SC * RA * SG * RAeom5CeoTeoTeo * Rm5Ceo | | SSRSR OOOR |
| WV- | m5Ceo * RAeo * RAeo * RGeo * RGeo * RG * SC * SA * SC * RA * | CAAGGGCACAGACTTCCAAA | RRRRR SSSRS |
| 13945 | SG * SA * SC * ST * ST * Rm5Ceo * Rm5Ceo * RAeo * RAeo * RAeo | | SSSSR RRRR |
| WV- | Ae * Rm5Ceo * RAeo * RAeo * RGeo * RG * SG * SC * SA * SC * | ACAAGGGCACAGACTTCCAA | RRRRR SSSSR |
| 13946 | RA * SG * SA * SC * ST * RTeo * Rm5Ceo * Rm5Ceo * RAeo * RAeo | | SSSSR RRRR |
| WV- | m5Ceo * RAeo * Rm5Ceo * RAeo * RAeo * RG * SG * SG * SC * SA * | CACAAGGGCACAGACTTCCA | RRRRR SSSSS |
| 13947 | SC * RA * SG * SA * SC * RTeo * RTeo * Rm5Ceo * Rm5Ceo * RAeo | | RSSSR RRRR |
| WV- | Geo * Rm5Ceo * RAeo * Rm5Ceo * RAeo * RA * SG * SG * SG * SC * | GCACAAGGGCA CAGACTTCC | RRRRR SSSSS |
| 13948 | SA * SC * RA * SG * SA * Rm5Ceo * RTeo * RTeo * Rm5Ceo * | | SRSSR RRRR |
| | Rm5Ceo | | |
| WV- | Geo * RGeo * Rm5Ceo * RAeo * Rm5Ceo * RA * SA * SG * SG * SG * | GGCACAAGGGCA CAGACTTC | RRRRR SSSSS |
| 13949 | SC * SA * SC * RA * SG * RAeo * Rm5Ceo * RTeo * RTeo * Rm5Ceo | | SSRSR RRRR |
| WV- | Aeo * Rm5Ceo * RAeo * RAeo * RGeo * RG * SG * SC * SG * SC * | ACAAGGGCGCAGACTTCCAA | RRRRR SSSSR |
| 13950 | RA * SG * SA * SC * ST * RTeo * Rm5Ceo * Rm5Ceo * RAeo * RAeo | | SSSSR RRRR |
| WV- | m5Ceo * RAeom5CeoAeoAeo * RG * SG * SG * SC * SG * RC * SA * | CACAAGGGCGCAGACTUCCA | ROOOR SSSSR |
| 13951 | SG * SA * SC * RTeo * SmU * SmC * SmC * SmA | | SSSSR SSSS |
| WV- | m5Ceo * RAeom5CeoAeoAeo * RG * SG * SG * SC * SG * SC * SA * | CACAAGGGCGCAGACTUCCA | ROOOR SSSSS |
| 13952 | RG * SA * SC * RTeo * SmU * SmC * SmC * SmA | | SRSSR SSSS |
| WV- | m5Ceo * RAeom5CeoAeoAeo * RG * SG * SG * SC * SA * RC * SA * | CACAAGGGCACAGACTUCCA | ROOOR SSSSR |
| 13953 | SG * SA * SC * RTeo * SmU * SmC * SmC * SmA | | SSSSR SSSS |
| WV- | m5Ceo * RAeom5CeoAeoAeo * RG * SG * SG * SC * SA * SC * SA * | CACAAGGGCACAGACTUCCA | ROOOR SSSSS |
| 13954 | RG * SA * SC * RTeo * SmU * SmC * SmC * SmA | | SRSSR SSSS |
| WV- | m5Ceo * RAeom5CeoAeoAeo * RG * SG * SG * SC * SG * RC * | CACAAGGGCGCAGACTTCCA | ROOOR SSSSR SSSSR |
| 13955 | SA * SG * SA * SC * RTeoTeom5Ceom5Ceo * RAeo | | OOOR |
| WV- | m5Ceo * RAeom5CeoAeoAeo * RG * SG * SG * SC * SG * SC * | CACAAGGGCGCAGACTTCCA | ROOOR SSSSS SRSSR |
| 13956 | SA * RG * SA * SC * RTeoTeom5Ceom5Ceo * RAeo | | OOOR |
| WV- | m5Ceo * RAeom5CeoAeoAeo * RG * SG * SG * SC * SA * RC * | CACAAGGGCACAGACTTCCA | ROOOR SSSSR SSSSR |
| 13957 | SA * SG * SA * SC * RTeoTeom5Ceom5Ceo * RAeo | | OOOR |
| WV- | m5Ceo * RAeom5CeoAeoAeo * RG * SG * SG * SC * SA * SC * | CACAAGGGCACAGACTTCCA | ROOOR SSSSS SRSSR |
| 13958 | SA * RG * SA * SC * RTeoTeom5Ceom5Ceo * RAeo | | OOOR |
| WV- | m5Ceo * RAeo * Rm5Ceo * RAeo * RAeo * RG * SG * SG * SC * SG * | CACAAGGGCGCAGACTTCCA | RRRRR SSSSR |
| 13959 | RC * SA * SG * SA * SC * RTeo * RTeo * Rm5Ceo * Rm5Ceo * RAeo | | SSSSR RRRR |
| WV- | m5Ceo * RAeo * Rm5Ceo * RAeo * RAeo * RG * SG * SG * SC * SG * | CACAAGGGCGCAGACTTCCA | RRRRR SSSSS |
| 13960 | SC * SA * RG * SA * SC * RTeo * RTeo * Rm5Ceo * Rm5Ceo * RAeo | | SRSSR RRRR |
| WV- | m5Ceo * RAeo * Rm5Ceo * RAeo * RAeo * RG * SG * SG * SC * SA * | CACAAGGGCACAGACTTCCA | RRRRR SSSSR |
| 13961 | RC * SA * SG * SA * SC * RTeo * RTeo * Rm5Ceo * Rm5Ceo * RAeo | | SSSSR RRRR |
| WV- | m5Ceo * RAeo * Rm5Ceo * RAeo * RAeo * RG * SG * SG * SC * SA * | CACAAGGGCACAGACTTCCA | RRRRR SSSSS |
| 13962 | SC * SA * RG * SA * SC * RTeo * RTeo * Rm5Ceo * Rm5Ceo * RAeo | | SRSSR RRRR |
| WV- | Aeo * RTeo * Rm5Ceo * RTeo * RGeo * RT * SA * RG * SC * SA * | ATCTGTAGCAGCAGCTTCTC | RRRRR SRSSS |
| 14059 | SG * SC * SA * SG * SC * RTeo * RTeo * Rm5Ceo * RTeo * Rm5Ceo | | SSSSR RRRR |
| WV- | Geo * RAeo * RTeo * Rm5Ceo * RTeo * RG * ST * SA * RG * SC * | GATCTGTAGCAGCAGCTTCT | RRRRR SSRSS |
| 14060 | SA * SG * SC * SA * SG * Rm5Ceo * RTeo * RTeo * Rm5Ceo * RTeo | | SSSSR RRRR |
| WV- | Teo * RGeo * RAeo * RTeo * Rm5Ceo * RT * SG * ST * SA * RG * SC | TGATCTGTAGCAGCAGCTTC | RRRRR SSSRS |
| 14061 | * SA * SG * SC * SA * RGeo * Rm5Ceo * RTeo * RTeo * Rm5Ceo | | SSSSR RRRR |
| WV- | Teo * RTeo * RGeo * RAeo * RTeo * RC * ST * SG * ST * SA * RG * | TTGATCTGTAGCAGCAGCTT | RRRRR SSSSR |
| 14062 | SC * SA * SG * SC * RAeo * RGeo * Rm5Ceo * RTeo * RTeo | | SSSSR RRRR |
| WV- | Geo * RTeo * RTeo * RGeo * RAeo * RT * SC * ST * SG * ST * SA * | GTTGATCTGTAGCAGCAGCT | RRRRR SSSSS |
| 14063 | RG * SC * SA * SG * Rm5Ceo * RAeo * RGeo * Rm5Ceo * RTeo | | RSSSR RRRR |
| WV- | Geo * RGeo * RTeo * RTeo * RGeo * RA * ST * SC * ST * SG * ST * | GGTTGATCTGTAGCAGCAGC | RRRRR SSSSS |
| 14064 | SA * RG * SC * SA * RGeo * Rm5Ceo * RAeo * RGeo * Rm5Ceo | | SRSSR RRRR |
| WV- | Geo * RGeo * RGeo * RTeo * RTeo * RG * SA * ST * SC * ST * SG * | GGGTTGATCTGTAGCAGCAG | RRRRR SSSSS |
| 14065 | ST * SA * RG * SC * RAeo * RGeo * Rm5Ceo * RAeo * RGeo | | SSRSR RRRR |
| WV- | Aeo * RTeom5CeoTeoGeo * RT * SA * RG * SC * SA * SG * SC | ATCTGTAGCAGCAGCTTCTC | ROOOR SRSSS SSSSR |
| 14066 | * SA * SG * SC * RTeo * RTeom5CeoTeom5Ceo | | ROOO |
| WV- | Geo * RAeoTeom5CeoTeo * RG * ST * SA * RG * SC * SA * SG | GATCTGTAGCAGCAGCTTCT | ROOOR SSRSS SSSSR |
| 14067 | * SC * SA * SG * Rm5CeoTeoTeom5Ceo * RTeo | | OOOR |
| WV- | Teo * RGeoAeoTeom5Ceo * RT * SG * ST * SA * RG * SC * SA | TGATCTGTAGCAGCAGCTTC | ROOOR SSSRS SSSSR |
| 14068 | * SG * SC * SA * RGeom5CeoTeoTeo * Rm5Ceo | | OOOR |
| WV- | Teo * RTeoGeoAeoTeo * RC * ST * SG * ST * SA * RG * SC * | TTGATCTGTAGCAGCAGCTT | ROOOR SSSSR SSSSR |
| 14069 | SA * SG * SC * RAeoGeom5CeoTeo * RTeo | | OOOR |
| WV- | Geo * RTeoTeoGeoAeo * RT * SC * ST * SG * ST * SA * RG * | GTTGATCTGTAGCAGCAGCT | ROOOR SSSSS RSSSR |
| 14070 | SC * SA * SG * Rm5CeoAeoGeom5Ceo * RTeo | | OOOR |
| WV- | Geo * RGeoTeoTeoGeo * RA * ST * SC * ST * SG * ST * SA * | GGTTGATCTGTAGCAGCAGC | ROOOR SSSSS SRSSR |
| 14071 | RG * SC * SA * RGeom5CeoAeoGeo * Rm5Ceo | | OOOR |
| WV- | Geo * RGeoGeoTeoTeo * RG * SA * ST * SC * ST * SG * ST * | GGGTTGATCTGTAGCAGCAG | ROOOR SSSSS SSRSR |
| 14072 | SA * RG * SC * RAeoGeom5CeoAeo * RGeo | | OOOR |
| WV- | Aeo * RTeom5CeoTeoGeo * RT * SA * RG * SC * SA * SG * SC | ATCTGTAGCAGCAGCUUCUC | ROOOR SRSSS SSSSR |
| 14073 | * SA * SG * SC * RmU * SmU * SmC * SmU * SmC | | SSSS |
| WV- | Geo * RAeoTeom5CeoTeo * RG * ST * SA * RG * SC * SA * SG | GATCTGTAGCAGCAGCUUCU | ROOOR SSRSS SSSSR |
| 14074 | * SC * SA * SG * RmC * SmU * SmU * SmC * SmU | | SSSS |
| WV- | Teo * RGeoAeoTeom5Ceo * RT * SG * ST * SA * RG * SC * SA | TGATCTGTAGCAGCAGCUUC | ROOOR SSSRS SSSSR |
| 14075 | * SG * SC * SA * RmG * SmC * SmU * SmU * SmC | | SSSS |
| WV- | Teo * RTeoGeoAeoTeo * RC * ST * SG * ST * SA * RG * SC * | TTGATCTGTAGCAGCAGCUU | ROOOR SSSSR SSSSR |
| 14076 | SA * SG * SC * RmA * SmG * SmC * SmU * SmU | | SSSS |
| WV- | Geo * RTeoTeoGeoAeo * RT * SC * ST * SG * ST * SA * RG * | GTTGATCTGTAGCAGCAGCU | ROOOR SSSSS RSSSR |
| 14077 | SC * SA * SG * RmC * SmA * SmG * SmC * SmU | | SSSS |
| WV- | Geo * RGeoTeoTeoGeo * RA * ST * SC * ST * SG * ST * SA * | GGTTGATCTGTAGCAGCAGC | ROOOR SSSSS SRSSR |
| 14078 | RG * SC * SA * RmG * SmC * SmA * SmG * SmC | | SSSS |
| WV- | Geo * RGeoGeoTeoTeo * RG * SA * ST * SC * ST * SG * ST * | GGGTTGATCTGTAGCAGCAG | ROOOR SSSSS SSRSR |
| 14079 | SA * RG * SC * RmA * SmG * SmC * SmA * SmG | | SSSS |
| WV- | Aeo * RTeom5CeoTeoGeo * RT * SA * RG * SC * SA * SG * SC | ATCTGTAGCAGCAGCTUCUC | ROOOR SRSSS SSSSR |
| 14080 | * SA * SG * SC * RTeo * SmU * SmC * SmU * SmC | | SSSS |
| WV- | Geo * RAeoTeom5CeoTeo * RG * ST * SA * RG * SC * SA * SG | GATCTGTAGCAGCAGCUUCU | ROOOR SSRSS SSSSR |
| 14081 | * SC * SA * SG * Rm5Ceo * SmU * SmU * SmC * SmU | | SSSS |
| WV- | Teo * RGeoAeoTeom5Ceo * RT * SG * ST * SA * RG * SC * SA | TGATCTGTAGCAGCAGCUUC | ROOOR SSSRS SSSSR |
| 14082 | * SG * SC * SA * RGeo * SmC * SmU * SmU * SmC | | SSSS |
| WV- | Teo * RTeoGeoAeoTeo * RC * ST * SG * ST * SA * RG * SC * | TTGATCTGTAGCAGCAGCUU | ROOOR SSSSR SSSSR |
| 14083 | SA * SG * SC * RAeo * SmG * SmC * SmU * SmU | | SSSS |
| WV- | Geo * RTeoTeoGeoAeo * RT * SC * ST * SG * ST * SA * RG * | GTTGATCTGTAGCAGCAGCU | ROOOR SSSSS RSSSR |
| 14084 | SC * SA * SG * Rm5Ceo * SmA * SmG * SmC * SmU | | SSSS |
| WV- | Geo * RGeoTeoTeoGeo * RA * ST * SC * ST * SG * ST * SA * | GGTTGATCTGTAGCAGCAGC | ROOOR SSSSS SRSSR |
| 14085 | RG * SC * SA * RGeo * SmC * SmA * SmG * SmC | | SSSS |
| WV- | Geo * RGeoGeoTeoTeo * RG * SA * ST * SC * ST * SG * ST * | GGGTTGATCTGTAGCAGCAG | ROOOR SSSSS SSRSR |
| 14086 | SA * RG * SC * RAeo * SmG * SmC * SmA * SmG | | SSSS |
| WV- | Geo * RTeo * RTeo * RGeo * RAeo * RT * SC * ST * SG * ST * | GTTGATCTGTAGCAGCAGCT | RRRRR SSSSR SSSSR |
| 14092 | RA * SG * SC * SA * SG * Rm5Ceo * RAeo * RGeo * Rm5Ceo * | | RRRR |
| | RTeo | | |
| WV- | Geo * RTeo * RTeo * RGeo * RAeo * RT * SC * ST * SG * ST * | GTTGATCTGTAGCAGCAGCT | RRRRR SSSSS SRSSR |
| 14093 | SA * SG * RC * SA * SG * Rm5Ceo * RAeo * RGeo * Rm5Ceo * | | RRRR |
| | RTeo | | |
| WV- | Geo * RTeoTeoGeoAeo * RT * SC * ST * SG * ST * RA * SG * | GTTGATCTGTAGCAGCAGCT | ROOOR SSSSR SSSSR |
| 14094 | SC * SA * SG * Rm5CeoAeoGeom5Ceo * RTeo | | OOOR |
| WV- | Geo * RTeoTeoGeoAeo * RT * SC * ST * SG * ST * SA * SG * | GTTGATCTGTAGCAGCAGCT | ROOOR SSSSS SRSSR |
| 14095 | RC * SA * SG * Rm5CeoAeoGeom5Ceo * RTeo | | OOOR |
| WV- | Geo * RTeoTeoGeoAeo * RT * SC * ST * SG * ST * RA * SG * | GTTGATCTGTAGCAGCAGCU | ROOOR SSSSR SSSSR |
| 14096 | SC * SA * SG * Rm5Ceo * SmA * SmG * SmC * SmU | | SSSS |
| WV- | Geo * RTeoTeoGeoAeo * RT * SC * ST * SG * ST * SA * SG * | GTTGATCTGTAGCAGCAGCU | ROOOR SSSSS SRSSR |
| 14097 | RC * SA * SG * Rm5Ceo * SmA * SmG * SmC * SmU | | SSSS |
| WV- | mG * SmU * SmU * SmG * SAeo * RT * SC * ST * SG * ST * RA | GUUGATCTGTAGCAGCAGCT | SSSSR SSSSR SSSSR |
| 14098 | * SG * SC * SA * SG * Rm5CeoAeoGeom5Ceo * STeo | | OOOS |
| WV- | mG * SmU * SmU * SmG * SAeo * RT * SC * ST * SG * ST * SA | GUUGATCTGTAGCAGCAGCT | SSSSR SSSSS SRSSR |
| 14099 | * SG * R * SA * SG * Rm5CeoAeoGeom5Ceo * STeo | | OOOS |
| WV- | mG * SmU * SmU * SmG * SAeo * ST * SC * ST * SG * ST * RA | GUUGATCTGTAGCAGCAGCT | SSSSS SSSSR SSSSR |
| 14100 | * SG * SC * SA * SG * Rm5CeoAeoGeom5Ceo * STeo | | OOOS |
| WV- | mG * SmU * SmU * SmG * SAeo * ST * SC * ST * SG * ST * SA | GUUGATCTGTAGCAGCAGCT | SSSSS SSSSS SRSSR |
| 14101 | * SG * RC * SA * SG * Rm5CeoAeoGeom5Ceo * STeo | | OOOS |
| WV- | mG * RmGmCmAmC * SA * SA * SG * SG * SG * SC * SA * SC | GGCACAAGGGCA | ROOOS SSSSS SSRSS |
| 14133 | * RA * SG * SmAmCmUmU * SmC | CAGACUUC | OOOS |
| WV- | mG * SmGmCmAmC * RA * SA * SG * SG * SG * SC * SA * SC | GGCACAAGGGCA | SOOOR SSSSS SSRSS |
| 14134 | * RA * SG * SmAmCmUmU * SmC | CAGACUUC | OOOS |
| WV- | mG * SmGmCmAmC * SA * SA * SG * SG * SG * SC * SA * SC | GGCACAAGGGCA | SOOOS SSSSS SSRSS |
| 14135 | * RA * SG * SmAmCmUmU * RmC | CAGACUUC | OOOR |
| WV- | mG * RmGmCmAmC * RA * SA * SG * SG * SG * SC * SA * | GGCACAAGGGCA | ROOOR SSSSS SSRSS |
| 14136 | SC * RA * SG * SmAmCmUmU * RmC | CAGACUUC | OOOR |
| WV- | mG * RmUmGmCmA * SC * SA * SC * SA * SG * ST * SA * SG | GUGCACACAGTAGATGAGGG | ROOOS SSSSS SSRSS |
| 14137 | * RA * ST * SmGmAmGmG * SmG | | OOOS |
| WV- | mG * SmUmGmCmA * RC * SA * SC * SA * SG * ST * SA * SG | GUGCACACAGTAGATGAGGG | SOOOR SSSSS SSRSS |
| 14138 | * RA * ST * SmGmAmGmG * SmG | | OOOS |
| WV- | mG * SmUmGmCmA * SC * SA * SC * SA * SG * ST * SA * SG | GUGCACACAGTAGATGAGGG | SOOOS SSSSS SSRSS |
| 14139 | * RA * ST * SmGmAmGmG * RmG | | OOOR |
| WV- | mG * RmUmGmCmA * RC * SA * SC * SA * SG * ST * SA * | GUGCACACAGTAGATGAGGG | ROOOR SSSSS SSRSS |
| 14140 | SG * RA * ST * SmGmAmGmG * RmG | | OOOR |
| WV- | Teo * m5Ceo * m5Ceo * Teo * m5Ceo * C * C * T * T * C * C * | TCCTCCCTTCCCTATGTACG | XXXXX XXXXX |
| 14213 | C * T * A * T * Geo * Teo * Aeo * m5Ceo * Geo | | XXXXX XXXX |
| WV- | rG rA rG rA rA rG rC rU rG rC rU rG rC rU rA rC rA rG rA rU rC | GAGAAGCUGCUGCUACAGAU | OOOOO OOOOO |
| 14712 | rA rA rC rC rC rC rG rA | CAACCCCGA | OOOOO OOOOO |
| | | | OOOOO OOO |
| WV- | rG rA rG rA rA rG rC rU rG rC rU rG rC rU rG rC rA rG rA rU rC | GAGAAGCUGCUGCUGCAGAU | OOOOO OOOOO |
| 14713 | rA rA rC rC rC rC rG rA | CAACCCCGA | OOOOO OOOOO |
| | | | OOOOO OOO |
| WV- | Aeo * RTeom5CeoTeoGeo * RT * SA * RG * SC * SA * SG * SC | ATCTGTAGCAGCAGCTTCTC | ROOOR SRSSS SSSSR |
| 14759 | * SA * SG * SC * RTeoTeom5CeoTeo * Rm5Ceo | | OOOR |
| WV- | mG * SmU * SmU * SmG * SmA * ST * SC * ST * SG * ST * RA | GUUGATCTGTAGCAGCAGCT | SSSSS SSSSR SSSSR |
| 14914 | * SG * SC * SA * SG * Rm5Ceon001Aeon001Geon001m5Ceo * | | nXnXnXS |
| | STeo | | |
| WV- | mG * SmUn001mUn001mGn001mA * ST * SC * ST * SG * ST * | GUUGATCTGTAGCAGCAGCT | SnXnXnXS SSSSR |
| 14915 | RA * SG * SC * SA * SG * Rm5CeoAeoGeom5Ceo * STeo | | SSSSR OOOS |
| WV- | mG * SmU * SmU * SmG * SmA * ST * SC * ST * SG * ST * RA | GUUGATCTGTAGCAGCAGCT | SSSSS SSSSR SRSSR |
| 15077 | * SG * RC * SA * SG * Rm5CeoAeoGeom5Ceo * STeo | | OOOS |
| WV- | mG * mU * mU * mG * mA * T * C * T * G * T * A * G * C * A | GUUGATCTGTAGCAGCAGCT | XXXXX XXXXX |
| 15078 | * G * m5CeoAeoGeom5Ceo * Teo | | XXXXX OOOX |
| WV- | mG * SmUn001mUn001mGn001mA * ST * SC * ST * SG * ST * | GUUGATCTGTAGCAGCAGCT | SnXnXnXS SSSSR |
| 15079 | RA * SG * RC * SA * SG * Rm5CeoAeoGeom5Ceo * STeo | | SRSSR OOOS |
| WV- | mG * SmU * SmU * SmG * SmA * ST * SC * ST * SG * ST * RA | GUUGATCTGTAGCAGCAGCT | SSSSS SSSSR SRSSR |
| 15080 | * SG * RC * SA * SG * Rm5Ceon001Aeon001Geon001m5Ceo * | | nXnXnXS |
| | STeo | | |
| WV- | mG * Sm5CeoAeom5CeomA * RC * SA * SG * ST * SA * SG * | GCACACAGTAGATGAGGGAG | SOOOR SSSSS SSSSS |
| 15133 | SA * ST * SG * SA * SmG * SmG * SmG * SmA * SmG | | SSSS |
| WV- | mG * Sm5CeoAeom5CeomA * SC * RA * SG * ST * SA * SG * | GCACACAGTAGATGAGGGAG | SOOOS RSSSS SSSSS |
| 15134 | SA * ST * SG * SA * SmG * SmG * SmG * SmA * SmG | | SSSS |
| WV- | mG * Sm5CeoAeom5CeomA * SC * SA * RG * ST * SA * SG * | GCACACAGTAGATGAGGGAG | SOOOS SRSSS SSSSS |
| 15135 | SA * ST * SG * SA * SmG * SmG * SmG * SmA * SmG | | SSSS |
| WV- | mG * Sm5CeoAeom5CeomA * SC * SA * SG * RT * SA * SG * | GCACACAGTAGATGAGGGAG | SOOOS SSRSS SSSSS |
| 15136 | SA * ST * SG * SA * SmG * SmG * SmG * SmA * SmG | | SSSS |
| WV- | mG * Sm5CeoAeom5CeomA * SC * SA * SG * ST * RA * SG * | GCACACAGTAGATGAGGGAG | SOOOS SSSRS SSSSS |
| 15137 | SA * ST * SG * SA * SmG * SmG * SmG * SmA * SmG | | SSSS |
| WV- | mG * Sm5CeoAeom5CeomA * SC * SA * SG * ST * SA * RG * | GCACACAGTAGATGAGGGAG | SOOOS SSSSR SSSSS |
| 15138 | SA * ST * SG * SA * SmG * SmG * SmG * SmA * SmG | | SSSS |
| WV- | mG * Sm5CeoAeom5CeomA * SC * SA * SG * ST * SA * SG * | GCACACAGTAGATGAGGGAG | SOOOS SSSSS SRSSS |
| 15139 | SA * RT * SG * SA * SmG * SmG * SmG * SmA * SmG | | SSSS |
| WV- | mG * Sm5CeoAeom5CeomA * SC * SA * SG * ST * SA * SG * | GCACACAGTAGATGAGGGAG | SOOOS SSSSS SSRSS |
| 15140 | SA * ST * RG * SA * SmG * SmG * SmG * SmA * SmG | | SSSS |
| WV- | mG * Sm5CeoAeom5CeomA * SC * SA * SG * ST * SA * SG * | GCACACAGTAGATGAGGGAG | SOOOS SSSSS SSSRS |
| 15141 | SA * ST * SG * RA * SmG * SmG * SmG * SmA * SmG | | SSSS |
| WV- | mG * Sm5CeoAeom5CeomA * SC * SA * SG * ST * SA * SG * | GCACACAGTAGATGAGGGAG | SOOOS SSSSS SSSSR |
| 15142 | SA * ST * SG * SA * RmG * SmG * SmG * SmA * SmG | | SSSS |
| WV- | m5Ceo * AeoTeoAeoTeo * C * C * C * C * T * C * C * T * C * T | CATATCCCCTCCTCTGCCAG | XOOOX XXXXX |
| 15213 | * mG * mC * mC * mA * mG | | XXXXX XXXX |
| WV- | rG rC rC rU rU rU rG rG rA rA rG rU rC rU rG rU rG rC rC rC rU | GCCUUUGGAAGUCUGUGCCC | OOOOO OOOOO |
| 15564 | rU rG rU rG rC rC rC rU rG rC | UUGUGCCCUGC | OOOOO OOOOO |
| | | | OOOOO OOOOO |
| WV- | m5Ceo * RAeon001m5Ceon001Aeon001Aeo * RG * SG * SG * SC * RG | CACAAGGGCGCAGACTTCCA | RnXnXnXR |
| 15630 | * SC * SA * SG * SA * SC * RTeo * RTeo * Rm5Ceo * Rm5Ceo * RAeo | | SSSRS SSSSR |
| | | | RRRR |
| WV- | m5Ceo * RAeon001m5Ceon001Aeon001Aeo * RG * SG * SG * SC * SG | CACAAGGGCGCAGACTTCCA | RnXnXnXR |
| 15631 | * RC * SA * SG * SA * SC * RTeo * RTeo * Rm5Ceo * Rm5Ceo * RAeo | | SSSSR SSSSR |
| | | | RRRR |
| WV- | m5Ceo * RAeon001m5Ceon001Aeon001Aeo * RG * SG * SG * SC * SG | CACAAGGGCGCAGACTTCCA | RnXnXnXR |
| 15632 | * SC * RA * SG * SA * SC * RTeo * RTeo * Rm5Ceo * Rm5Ceo * RAeo | | SSSSS RSSSR |
| | | | RRRR |
| WV- | m5Ceo * RAeon001m5Ceon001Aeon001Aeo * RG * SG * SG * SC * SG | CACAAGGGCGCAGACTTCCA | RnXnXnXR |
| 15633 | * SC * SA * RG * SA * SC * RTeo * RTeo * Rm5Ceo * Rm5Ceo * RAeo | | SSSSS SRSSR |
| | | | RRRR |
| WV- | m5Ceo * RAeon001m5Ceon001Aeon001Aeo * RG * SG * SG * SC * RA | CACAAGGGCACAGACTTCCA | RnXnXnXR |
| 15634 | * SC * SA * SG * SA * SC * RTeo * RTeo * Rm5Ceo * Rm5Ceo * RAeo | | SSSRS SSSSR |
| | | | RRRR |
| WV- | m5Ceo * RAeon001m5Ceon001Aeon001Aeo * RG * SG * SG * SC * SA | CACAAGGGCACAGACTTCCA | RnXnXnXR |
| 15635 | * RC * SA * SG * SA * SC * RTeo * RTeo * Rm5Ceo * Rm5Ceo * RAeo | | SSSSR SSSSR |
| | | | RRRR |
| WV- | m5Ceo * RAeon001m5Ceon001Aeon001Aeo * RG * SG * SG * SC * SA | CACAAGGGCACAGACTTCCA | RnXnXnXR |
| 15636 | * SC * RA * SG * SA * SC * RTeo * RTeo * Rm5Ceo * Rm5Ceo * RAeo | | SSSSS RSSSR |
| | | | RRRR |
| WV- | m5Ceo * RAeon001m5Ceon001Aeon001Aeo * RG * SG * SG * SC * SA | CACAAGGGCACAGACTTCCA | RnXnXnXR |
| 15637 | * SC * SA * RG * SA * SC * RTeo * RTeo * Rm5Ceo * Rm5Ceo * RAeo | | SSSSS SRSSR |
| | | | RRRR |
| WV- | m5Ceo * RAeon001m5Ceon001Aeon001Aeo * RG * SG * SG * SC * RG | CACAAGGGCGCAGACUUCCA | RnXnXnXR |
| 15638 | * SC * SA * SG * SA * SC * SmU * SmU * SmC * SmC * SmA | | SSSRS SSSSS |
| | | | SSSS |
| WV- | m5Ceo * RAeon001m5Ceon001Aeon001Aeo * RG * SG * SG * SC * SG | CACAAGGGCGCAGACUUCCA | RnXnXnXR |
| 15639 | * RC * SA * SG * SA * SC * SmU * SmU * SmC * SmC * SmA | | SSSSR SSSSS |
| | | | SSSS |
| WV- | m5Ceo * RAeon001m5Ceon001Aeon001Aeo * RG * SG * SG * SC * SG | CACAAGGGCGCAGACUUCCA | RnXnXnXR |
| 15640 | * SC * RA * SG * SA * SC * SmU * SmU * SmC * SmC * SmA | | SSSSS RSSSS |
| | | | SSSS |
| WV- | m5Ceo * RAeon001m5Ceon001Aeon001Aeo * RG * SG * SG * SC * SG | CACAAGGGCGCAGACUUCCA | RnXnXnXR |
| 15641 | * SC * SA * RG * SA * SC * SmU * SmU * SmC * SmC * SmA | | SSSSS SRSSS |
| | | | SSSS |
| WV- | m5Ceo * RAeon001m5Ceon001Aeon001Aeo * RG * SG * SG * SC * RA | CACAAGGGCACAGACUUCCA | RnXnXnXR |
| 15642 | * SC * SA * SG * SA * SC * SmU * SmU * SmC * SmC * SmA | | SSSRS SSSSS |
| | | | SSSS |
| WV- | m5Ceo * RAeon001m5Ceon001Aeon001Aeo * RG * SG * SG * SC * SA | CACAAGGGCACAGACUUCCA | RnXnXnXR |
| 15643 | * RC * SA * SG * SA * SC * SmU * SmU * SmC * SmC * SmA | | SSSSR SSSSS |
| | | | SSSS |
| WV- | m5Ceo * RAeon001m5Ceon001Aeon001Aeo * RG * SG * SG * SC * SA | CACAAGGGCACAGACUUCCA | RnXnXnXR |
| 15644 | * SC * RA * SG * SA * SC * SmU * SmU * SmC * SmC * SmA | | SSSSS RSSSS |
| | | | SSSS |
| WV- | m5Ceo * RAeon001m5Ceon001Aeon001Aeo * RG * SG * SG * SC * SA | CACAAGGGCACAGACUUCCA | RnXnXnXR |
| 15645 | * SC * SA * RG * SA * SC * SmU * SmU * SmC * SmC * SmA | | SSSSS SRSSS |
| | | | SSSS |
| WV- | PSmC * RG * RC * RC * RA | CGCCA | RRRR |
| 16213 | | | |
| WV- | mG * SmU * SmU * SmG * SmA * ST * SC * ST * SG * ST * RA * SG * | GUUGATCTGTAGCAGCAGCT | SSSSS SSSSR |
| 16214 | SC * SA * SG * Sm5Ceon001SAeon001SGeon001Sm5Ceo * STeo | | SSSSS nSnSnSS |
| WV- | mG * SmU * SmU * SmG * SmA * ST * SC * ST * SG * RT * SA * SG * | GUUGATCTGTAGCAGCAGCT | SSSSS SSSRS |
| 16215 | SC * SA * SG * Sm5Ceon001SAeon001SGeon001Sm5Ceo * STeo | | SSSSS nSnSnSS |
| WV- | mG * SmU * SmU * SmG * SmA * ST * SC * ST * SG * ST * RA * SG * | GUUGATCTGTAGCAGCAGCT | SSSSS SSSSR |
| 16216 | RC * SA * SG * Sm5Ceon001SAeon001SGeon001Sm5Ceo * STeo | | SRSSS nSnSnSS |
| WV- | mG * SmU * SmU * SmG * SmA * ST * SC * ST * SG * RT * SA * SG * | GUUGATCTGTAGCAGCAGCT | SSSSS SSSRS |
| 16217 | SC * SA * SG * Sm5CeoAeoGeom5Ceo * STeo | | SSSSS OOOS |
| WV- | mG * SmU * SmU * SmG * SmA * ST * SC * ST * SG * ST * RA * SG * | GUUGATCTGTAGCAGCAGCT | SSSSS SSSSR |
| 16218 | SC * SA * SG * Sm5CeoAeoGeom5Ceo * STeo | | SSSSS OOOS |
| WV- | mC * mC * mU * mU * mC * T * T * C * A * T * T * C * T * T * G * | CCUUCTTCATTCTTGCCCAA | XXXXX XXXXX |
| 17213 | m5Ceom5Ceom5CeoAeo * Aeo | | XXXXX OOOX |
| WV- | mG * SmUn001mUn001mGn001mA * ST * SC * ST * SG * ST * RA | GUUGATCTGTAGCAGCAGCT | SnXnXnXS SSSSR |
| 17776 | * SG * SC * SA * SG * Rm5Ceon001Aeon001Geon001m5Ceo * STeo | | SSSSR nXnXnXS |
| WV- | mG * SmU * SmUn001mG * SmA * ST * SC * ST * SG * ST * RA * | GUUGATCTGTAGCAGCAGCT | SSnXSS SSSSR |
| 17777 | SG * SC * SA * SG * Rm5CeoAeoGeom5Ceo * STeo | | SSSSR OOOS |
| WV- | mG * SmU * SmU * SmG * SmA * ST * SC * ST * SG * ST * RA * | GUUGATCTGTAGCAGCAGCT | SSSSS SSSSR |
| 17778 | SG * SC * SA * SG * Rm5CeoAeon001Geom5Ceo * STeo | | SSSSR OnXOS |
| WV- | mG * SmU * SmUn001mG * SmA * ST * SC * ST * SG * ST * RA * | GUUGATCTGTAGCAGCAGCT | SSnXSS SSSSR |
| 17779 | SG * SC * SA * SG * Rm5CeoAeon001Geom5Ceo * STeo | | SSSSR OnXOS |
| WV- | mG * SmUn001mU * SmGn001mA * ST * SC * ST * SG * ST * RA * | GUUGATCTGTAGCAGCAGCT | SnXSnXS SSSSR |
| 17780 | SG * SC * SA * SG * Rm5CeoAeoGeom5Ceo * STeo | | SSSSR OOOS |
| WV- | mG * SmU * SmU * SmG * SmA * ST * SC * ST * SG * ST * RA * | GUUGATCTGTAGCAGCAGCT | SSSSS SSSSR |
| 17781 | SG * SC * SA * SG * Rm5Ceon001AeoGeon001m5Ce * STeo | | SSSSR nXOnXS |
| WV- | mG * SmUn001mU * SmGn001mA * ST * SC * ST * SG * ST * RA * | GUUGATCTGTAGCAGCAGCT | SnXSnXS SSSSR |
| 17782 | SG * SC * SA * SG * Rm5Ceon001AeoGeon001m5Ce * STeo | | SSSSR nXOnXS |
| WV- | Geo * RTeon001Teon001Geon001Aeo * RT * SC * ST * SG * ST * | GTTGATCTGTAGCAGCAGCU | RnXnXnXR SSSSS |
| 17783 | SA * RG * SC * SA * SG * Rm5Ceo * SmA * SmG * SmC * SmU | | RSSSR SSSS |
| WV- | Geo * RTeoTeoGeoAeo * RT * SC * ST * SG * ST * SA * RG * SC * | GTTGATCTGTAGCAGCAGCU | ROOOR SSSSS |
| 17784 | SA * SG * Rm5Ceon001mAn001mGn001mC * SmU | | RSSSR nXnXnXS |
| WV- | Geo * RTeon001Teon001Geon001Aeo * RT * SC * ST * SG * ST * | GTTGATCTGTAGCAGCAGCU | RnXnXnXR SSSSS |
| 17785 | SA * RG * SC * SA * SG * Rm5Ceon001mAn001mGn001mC * SmU | | RSSSR nXnXnXS |
| WV- | Geo * RTeoTeon001GeoAeo * RT * SC * ST * SG * ST * SA * RG * | GTTGATCTGTAGCAGCAGCU | ROnXOR SSSSS |
| 17786 | SC * SA * SG * Rm5Ceo * SmA * SmG * SmC * SmU | | RSSSR SSSS |
| WV- | Geo * RTeoTeoGeoAeo * RT * SC * ST * SG * ST * SA * RG * SC * | GTTGATCTGTAGCAGCAGCU | ROOOR SSSSS |
| 17787 | SA * SG * Rm5Ceo * SmAn001mG * SmC * SmU | | RSSSR SnXSS |
| WV- | Geo * RTeoTeon001GeoAeo * RT * SC * ST * SG * ST * SA * RG * | GTTGATCTGTAGCAGCAGCU | ROnXOR SSSSS |
| 17788 | SC * SA * SG * Rm5Ceo * SmAn001mG * SmC * SmU | | RSSSR SnXSS |
| WV- | Geo * RTeon001TeoGeon001Aeo * RT * SC * ST * SG * ST * SA * | GTTGATCTGTAGCAGCAGCU | RnXOnXR SSSSS |
| 17789 | RG * SC * SA * SG * Rm5Ceo * SmA * SmG * SmC * SmU | | RSSSR SSSS |
| WV- | Geo * RTeoTeoGeoAeo * RT * SC * ST * SG * ST * SA * RG * SC * | GTTGATCTGTAGCAGCAGCU | ROOOR SSSSS |
| 17790 | SA * SG * Rm5Ceon001mA * SmGn001mC * SmU | | RSSSR nXSnXS |
| WV- | Geo * RTeon001TeoGeon001Aeo * RT * SC * ST * SG * ST * SA * | GTTGATCTGTAGCAGCAGCU | RnXOnXR SSSSS |
| 17791 | RG * SC * SA * SG * Rm5Ceon001mA * SmGn001mC * SmU | | RSSSR nXSnXS |
| WV- | Geo * RTeon001Teon001Geon001Aeo * RT * SC * ST * SG * ST * | GTTGATCTGTAGCAGCAGCU | RnXnXnXR SSSSS |
| 17792 | SA * RG * SC * SA * SG * SmC * SmA * SmG * SmC * SmU | | RSSSS SSSS |
| WV- | Geo * RTeoTeoGeoAeo * RT * SC * ST * SG * ST * SA * RG * SC * | GTTGATCTGTAGCAGCAGCU | ROOOR SSSSS |
| 17793 | SA * SG * SmCn001mAn001mGn001mC * SmU | | RSSSS nXnXnXS |
| WV- | Geo * RTeon001Teon001Geon001Aeo * RT * SC * ST * SG * ST * | GTTGATCTGTAGCAGCAGCU | RnXnXnXR SSSSS |
| 17794 | SA * RG * SC * SA * SG * SmCn001mAn001mGn001mC * SmU | | RSSSS nXnXnXS |
| WV- | Geo * RTeoTeon001GeoAeo * RT * SC * ST * SG * ST * SA * RG * | GTTGATCTGTAGCAGCAGCU | ROnXOR SSSSS |
| 17795 | SC * SA * SG * SmC * SmA * SmG * SmC * SmU | | RSSSS SSSS |
| WV- | Geo * RTeoTeoGeoAeo * RT * SC * ST * SG * ST * SA * RG * SC * | GTTGATCTGTAGCAGCAGCU | ROOOR SSSSS |
| 17796 | SA * SG * SmC * SmAn001mG * SmC * SmU | | RSSSS SnXSS |
| WV- | Geo * RTeoTeon001GeoAeo * RT * SC * ST * SG * ST * SA * RG * | GTTGATCTGTAGCAGCAGCU | ROnXOR SSSSS |
| 17797 | SC * SA * SG * SmC * SmAn001mG * SmC * SmU | | RSSSS SnXSS |
| WV- | Geo * RTeon001TeoGeon001Aeo * RT * SC * ST * SG * ST * SA * | GTTGATCTGTAGCAGCAGCU | RnXOnXR SSSSS |
| 17798 | RG * SC * SA * SG * SmC * SmA * SmG * SmC * SmU | | RSSSS SSSS |
| WV- | Geo * RTeoTeoGeoAeo * RT * SC * ST * SG * ST * SA * RG * SC * | GTTGATCTGTAGCAGCAGCU | ROOOR SSSSS |
| 17799 | SA * SG * SmCn001mA * SmGn001mC * SmU | | RSSSS nXSnXS |
| WV- | Geo * RTeon001TeoGeon001Aeo * RT * SC * ST * SG * ST * SA * | GTTGATCTGTAGCAGCAGCU | RnXOnXR SSSSS |
| 17800 | RG * SC * SA * SG * SmCn001mA * SmGn001mC * SmU | | RSSSS nXSnXS |
| WV- | mC * SAeon001Aeon001Geon001mG * SG * C * G * C * A * G * A * | CAAGGGCGCAGACTTCCAAA | SnXnXnXS XXXXX |
| 17886 | C * T * T * SmC * SmC * SmA * SmA * SmA | | XXXXS SSSS |
| WV- | mC * SAeon001m5Ceon001Aeon001mA * SG * G * G * C * G * C * | CACAAGGGCGCAGACUUCCA | SnXnXnXS XXXXX |
| 17887 | A * G * A * C * SmU * SmU * SmC * SmC * SmA | | XXXXS SSSS |
| WV- | mG * SGeon001m5Ceon001Aeon001mC * SA * A * G * G * G * C * | GGCACAAGGGCGCAGACUUC | SnXnXnXS XXXXX |
| 17888 | G * C * A * G * SmA * SmC * SmU * SmU * SmC | | XXXXS SSSS |
| WV- | mC * SAeon001Aeon001Geon001Geo * RG * C * G * C * A * G * A | CAAGGGCGCAGACTTCCAAA | SnXnXnXR XXXXX |
| 17889 | * C * T * T * SmC * SmC * SmA * SmA * SmA | | XXXXS SSSS |
| WV- | mC * SAeon001m5Ceon001Aeon001Aeo * RG * G * G * C * G * C * | CACAAGGGCGCAGACUUCCA | SnXnXnXR XXXXX |
| 17890 | A * G * A * C * SmU * SmU * SmC * SmC * SmA | | XXXXS SSSS |
| WV- | mG * SGeon001m5Ceon001Aeon001m5Ceo * RA * A * G * G * G * | GGCACAAGGGCGCAGACUUC | SnXnXnXR XXXXX |
| 17891 | C * G * C * A * G * SmA * SmC * SmU * SmU * SmC | | XXXXS SSSS |
| WV- | m5Ceo * RAeon001Aeon001Geon001Geo * RG * C * G * C * A * G * | CAAGGGCGCAGACTTCCAAA | RnXnXnXR |
| 17892 | A * C * T * T * SmC * SmC * SmA * SmA * SmA | | XXXXX XXXXS |
| | | | SSSS |
| WV- | m5Ceo * RAeon001m5Ceon001Aeon001Aeo * RG * G * G * C * G * | CACAAGGGCGCAGACUUCCA | RnXnXnXR |
| 17893 | C * A * G * A * C * SmU * SmU * SmC * SmC * SmA | | XXXXX XXXXS |
| | | | SSSS |
| WV- | Geo * RGeon001m5Ceon001Aeon001m5Ceo * RA * A * G * G * G * | GGCACAAGGGCGCAGACUUC | RnXnXnXR |
| 17894 | C * G * C * A * G * SmA * SmC * SmU * SmU * SmC | | XXXXX XXXXS |
| | | | SSSS |
| WV- | mC * SAeon001Aeon001Geon001mG * SG * Sm5C * RA * SC * SA * | CAAGGGCACAGACTTCCAAA | SnXnXnXS SRSSS |
| 17895 | SG * SA * SC * ST * ST * SmC * SmC * SmA * SmA * SmA | | SSSSS SSSS |
| WV- | mC * SAeon001Aeon001Geon001mG * SG * Sm5C * SA * RC * SA * | CAAGGGCACAGACTTCCAAA | SnXnXnXS SSRSS |
| 17896 | SG * SA * SC * ST * ST * SmC * SmC * SmA * SmA * SmA | | SSSSS SSSS |
| WV- | mC * SAeon001Aeon001Geon001mG * SG * Sm5C * SA * SC * RA * | CAAGGGCACAGACTTCCAAA | SnXnXnXS SSSRS |
| 17897 | SG * SA * SC * ST * ST * SmC * SmC * SmA * SmA * SmA | | SSSSS SSSS |
| WV- | mC * SAeon001Aeon001Geon001mG * SG * Sm5C * SA * SC * SA * | CAAGGGCACAGACTTCCAAA | SnXnXnXS SSSSR |
| 17898 | RG * SA * SC * ST * ST * SmC * SmC * SmA * SmA * SmA | | SSSSS SSSS |
| WV- | mA * Sm5Ceon001Aeon001Aeon001mG * SG * SG * Rm5C * SA * | ACAAGGGCACAGACTUCCAA | SnXnXnXS SRSSS |
| 17899 | SC * SA * SG * SA * SC * ST * SmU * SmC * SmC * SmA * SmA | | SSSSS SSSS |
| WV- | mA * Sm5Ceon001Aeon001Aeon001mG * SG * SG * Sm5C * RA * | ACAAGGGCACAGACTUCCAA | SnXnXnXS SSRSS |
| 17900 | SC * SA * SG * SA * SC * ST * SmU * SmC * SmC * SmA * SmA | | SSSSS SSSS |
| WV- | mA * Sm5Ceon001Aeon001Aeon001mG * SG * SG * Sm5C * SA * | ACAAGGGCACAGACTUCCAA | SnXnXnXS SSSRS |
| 17901 | RC * SA * SG * SA * SC * ST * SmU * SmC * SmC * SmA * SmA | | SSSSS SSSS |
| WV- | mA * Sm5Ceon001Aeon001Aeon001mG * SG * SG * Sm5C * SA * | ACAAGGGCACAGACTUCCAA | SnXnXnXS SSSSR |
| 17902 | SC * RA * SG * SA * SC * ST * SmU * SmC * SmC * SmA * SmA | | SSSSS SSSS |
| WV- | mA * Sm5Ceon001Aeon001Aeon001mG * SG * SG * Sm5C * SA * | ACAAGGGCACAGACTUCCAA | SnXnXnXS SSSSS |
| 17903 | SC * SA * RG * SA * SC * ST * SmU * SmC * SmC * SmA * SmA | | RSSSS SSSS |
| WV- | mC * SAeon001m5Ceon001Aeon001mA * SG * SG * RG * Sm5C * | CACAAGGGCACAGACUUCCA | SnXnXnXS SRSSS |
| 17904 | SA * SC * SA * SG * SA * SC * SmU * SmU * SmC * SmC * SmA | | SSSSS SSSS |
| WV- | mC * SAeon001m5Ceon001Aeon001mA * SG * SG * SG * Rm5C * | CACAAGGGCACAGACUUCCA | SnXnXnXS SSRSS |
| 17905 | SA * SC * SA * SG * SA * SC * SmU * SmU * SmC * SmC * SmA | | SSSSS SSSS |
| WV- | mC * SAeon001m5Ceon001Aeon001mA * SG * SG * SG * Sm5C * | CACAAGGGCACAGACUUCCA | SnXnXnXS SSSRS |
| 17906 | RA * SC * SA * SG * SA * SC * SmU * SmU * SmC * SmC * SmA | | SSSSS SSSS |
| WV- | mC * SAeon001m5Ceon001Aeon001mA * SG * SG * SG * Sm5C * | CACAAGGGCACAGACUUCCA | SnXnXnXS SSSSR |
| 17907 | SA * RC * SA * SG * SA * SC * SmU * SmU * SmC * SmC * SmA | | SSSSS SSSS |
| WV- | mC * SAeon001m5Ceon001Aeon001mA * SG * SG * SG * Sm5C * | CACAAGGGCACAGACUUCCA | SnXnXnXS SSSSS |
| 17908 | SA * SC * RA * SG * SA * SC * SmU * SmU * SmC * SmC * SmA | | RSSSS SSSS |
| WV- | mC * SAeon001m5Ceon001Aeon001mA * SG * SG * SG * Sm5C * | CACAAGGGCACAGACUUCCA | SnXnXnXS SSSSS |
| 17909 | SA * SC * SA * RG * SA * SC * SmU * SmU * SmC * SmC * SmA | | SRSSS SSSS |
| WV- | mG * Sm5Ceon001Aeon001m5Ceon001mA * SA * SG * SG * RG * | GCACAAGGGCA | SnXnXnXS SSRSS |
| 17910 | Sm5C * SA * SC * SA * SG * SA * SmC * SmU * SmU * SmC * | CAGACUUCC | SSSSS SSSS |
| | SmC | | |
| WV- | mG * Sm5Ceon001Aeon001m5Ceon001mA * SA * SG * SG * SG * | GCACAAGGGCA | SnXnXnXS SSSRS |
| 17911 | Rm5C * SA * SC * SA * SG * SA * SmC * SmU * SmU * SmC * | CAGACUUCC | SSSSS SSSS |
| | SmC | | |
| WV- | mG * Sm5Ceon001Aeon001m5Ceon001mA * SA * SG * SG * SG * | GCACAAGGGCA | SnXnXnXS SSSSR |
| 17912 | Sm5C * RA * SC * SA * SG * SA * SmC * SmU * SmU * SmC * | CAGACUUCC | SSSSS SSSS |
| | SmC | | |
| WV- | mG * Sm5Ceon001Aeon001m5Ceon001mA * SA * SG * SG * SG * | GCACAAGGGCA | SnXnXnXS SSSSS |
| 17913 | 5m5C * SA * RC * SA * SG * SA * SmC * SmU * SmU * SmC * | CAGACUUCC | RSSSS SSSS |
| | SmC | | |
| WV- | mG * Sm5Ceon001Aeon001m5Ceon001mA * SA * SG * SG * SG * | GCACAAGGGCA | SnXnXnXS SSSSS |
| 17914 | 5m5C * SA * SC * RA * SG * SA * SmC * SmU * SmU * SmC * | CAGACUUCC | SRSSS SSSS |
| | SmC | | |
| WV- | mG * SGeon001m5Ceon001Aeon001mC * SA * SA * SG * SG * RG | GGCACAAGGGCA | SnXnXnXS SSSRS |
| 17915 | * Sm5C * SA * SC * SA * SG * SmA * SmC * SmU * SmU * SmC | CAGACUUC | SSSSS SSSS |
| WV- | mG * SGeon001m5Ceon001Aeon001mC * SA * SA * SG * SG * SG * | GGCACAAGGGCA | SnXnXnXS SSSSR |
| 17916 | Rm5C * SA * SC * SA * SG * SmA * SmC * SmU * SmU * SmC | CAGACUUC | SSSSS SSSS |
| WV- | mG * SGeon001m5Ceon001Aeon001mC * SA * SA * SG * SG * SG * | GGCACAAGGGCA | SnXnXnXS SSSSS |
| 17917 | Sm5C * RA * SC * SA * SG * SmA * SmC * SmU * SmU * SmC | CAGACUUC | RSSSS SSSS |
| WV- | mG * SGeon001m5Ceon001Aeon001mC * SA * SA * SG * SG * SG * | GGCACAAGGGCA | SnXnXnXS SSSSS |
| 17918 | Sm5C * SA * RC * SA * SG * SmA * SmC * SmU * SmU * SmC | CAGACUUC | SRSSS SSSS |
| WV- | mG * mU * mC * mC * mU * C * A * A * T * G * C * C * C * C * | GUCCUCAATGCCCCAGGGTT | XXXXX XXXXX |
| 18213 | A * GeoGeoGeoTeo * Teo | | XXXXX OOOX |
| WV- | m5Ceo * Aeon001Geon001Teon001Teo * T * G * G * G * C * A * | CAGTTTGGGCATTTTGTCCG | XnXnXnXX XXXXX |
| 19213 | T * T * T * T * GeoTeom5Ceom5Ceo * Geo | | XXXXX OOOX |
| WV- | mG * SmU * SmU * SmG * SAeo * RT * SC * ST * SG * ST * RA | GUUGATCTGTAGCAGCAGCT | SSSSR SSSSR SSSSR |
| 19819 | * SG * SC * SA * SG * Rm5CeoAeoGeom5Ceo * RTeo | | OOOR |
| WV- | mG * SmU * SmU * SmG * SAeo * RT * SC * ST * SG * ST * RA * | GUUGATCTGTAGCAGCAGCT | SSSSR SSSSR |
| 19820 | SG * SC * SA * SG * Rm5Ceon001Aeon001Geon001m5Ceo * RTeo | | SSSSR nXnXnXR |
| WV- | mG * SmU * SmU * SmG * SAeo * RT * SC * ST * SG * ST * RA * | GUUGATCTGTAGCAGCAGCT | SSSSR SSSSR |
| 19821 | SG * SC * SA * SG * Rm5Ceon001RAeon001RGeon001Rm5Ceo * | | SSSSR nRnRnRR |
| | RTeo | | |
| WV- | mG * SmU * SmU * SmG * SAeo * RT * SC * ST * SG * ST * RA * | GUUGATCTGTAGCAGCAGCT | SSSSR SSSSR |
| 19822 | SG * SC * SA * SG * Rm5Ceon001SAeon001SGeon001Sm5Ceo * | | SSSSR nSnSnSR |
| | RTeo | | |
| WV- | mG * SmU * SmU * SmG * SAeo * RT * SCn001T * SG * ST * RA * | GUUGATCTGTAGCAGCAGCT | SSSSR SnXSSR |
| 19823 | SG * SCn001A * SG * Rm5Ceon001Aeon001Geon001m5Ceo * RTeo | | SSnXSR nXnXnXR |
| WV- | mG * SmU * SmU * SmG * SAeo * RT * SCn001T * SG * ST * RA * | GUUGATCTGTAGCAGCAGCT | SSSSR SnXSSR |
| 19824 | SG * SC * SA * SG * Rm5Ceon001Aeon001Geon001m5Ceo * RTeo | | SSSSRnXnXnXR |
| WV- | mG * SmU * SmU * SmG * SAeo * RT * SC * ST * SG * ST * RA * | GUUGATCTGTAGCAGCAGCT | SSSSR SSSSR |
| 19825 | SG * SCn001A * SG * Rm5Ceon001Aeon001Geon001m5Ceo * RTeo | | SSnXSR nXnXnXR |
| WV- | mG * SmU * SmU * SmG * SAeo * RT * SCn001RT * SG * ST * RA * | GUUGATCTGTAGCAGCAGCT | SSSSR SnRSSR |
| 19826 | SG * SCn001RA * SG * Rm5Ceon001RAeon001RGeon001Rm5Ceo * | | SSnRSR nRnRnRR |
| | RTeo | | |
| WV- | mG * SmU * SmU * SmG * SAeo * RT * SCn001RT * SG * ST * RA * | GUUGATCTGTAGCAGCAGCT | SSSSR SnRSSR |
| 19827 | SG * SC * SA * SG * Rm5Ceon001RAeon001RGeon001Rm5Ceo * | | SSSSR nRnRnRR |
| | RTeo | | |
| WV- | mG * SmU * SmU * SmG * SAeo * RT * SC * ST * SG * ST * RA * SG | GUUGATCTGTAGCAGCAGCT | SSSSR SSSSR |
| 19828 | * SCn001RA * SG * Rm5Ceon001RAeon001RGeon001Rm5Ceo * RTeo | | SSnRSR nRnRnRR |
| WV- | mG * SmU * SmU * SmG * SAeo * RT * SCn001ST * SG * ST * RA * | GUUGATCTGTAGCAGCAGCT | SSSSR SnSSSR |
| 19829 | SG * SCn001SA * SG * Rm5Ceon001RAeon001RGeon001Rm5Ceo * | | SSnSSR nRnRnRR |
| | RTeo | | |
| WV- | mG * SmU * SmU * SmG * SAeo * RT * SCn001ST * SG * ST * RA * | GUUGATCTGTAGCAGCAGCT | SSSSR SnSSSR |
| 19830 | SG * SC * SA * SG * Rm5Ceon001RAeon001RGeon001Rm5Ceo * | | SSSSR nRnRnRR |
| | RTeo | | |
| WV- | mG * SmU * SmU * SmG * SAeo * RT * SC * ST * SG * ST * RA * SG | GUUGATCTGTAGCAGCAGCT | SSSSR SSSSR |
| 19831 | * SCn001SA * SG * Rm5Ceon001RAeon001RGeon001Rm5Ceo * RTeo | | SSnSSR nRnRnRR |
| WV- | mG * SmU * SmU * SmG * SAeo * RT * SCn001RT * SG * ST * RA * | GUUGATCTGTAGCAGCAGCT | SSSSR SnRSSR |
| 19832 | SG * SCn001RA * SG * Rm5Ceon001SAeon001SGeon001Sm5Ceo * | | SSnRSR nSnSnSR |
| | RTeo | | |
| WV- | mG * SmU * SmU * SmG * SAeo * RT * SCn001RT * SG * ST * RA * | GUUGATCTGTAGCAGCAGCT | SSSSR SnRSSR |
| 19833 | SG * SC * SA * SG * Rm5Ceon001SAeon001SGeon001Sm5Ceo * RTeo | | SSSSR nSnSnSR |
| WV- | mG * SmU * SmU * SmG * SAeo * RT * SC * ST * SG * ST * RA * SG | GUUGATCTGTAGCAGCAGCT | SSSSR SSSSR |
| 19834 | * SCn001RA * SG * Rm5Ceon001SAeon001SGeon001Sm5Ceo * RTeo | | SSnRSR nSnSnSR |
| WV- | mG * SmU * SmU * SmG * SAeo * RT * SCn001ST * SG * ST * RA * | GUUGATCTGTAGCAGCAGCT | SSSSR SnSSSR |
| 19835 | SG * SCn001SA * SG * Rm5Ceon001SAeon001SGeon001Sm5Ceo * | | SSnSSR nSnSnSR |
| | RTeo | | |
| WV- | mG * SmU * SmU * SmG * SAeo * RT * SCn001ST * SG * ST * RA * | GUUGATCTGTAGCAGCAGCT | SSSSR SnSSSR |
| 19836 | SG * SC * SA * SG * Rm5Ceon001SAeon001SGeon001Sm5Ceo * RTeo | | SSSSR nSnSnSR |
| WV- | mG * SmU * SmU * SmG * SAeo * RT * SC * ST * SG * ST * RA * SG | GUUGATCTGTAGCAGCAGCT | SSSSR SSSSR |
| 19837 | * SCn001SA * SG * Rm5Ceon001SAeon001SGeon001Sm5Ceo * RTeo | | SSnSSR nSnSnSR |
| WV- | Geo * RTeon001Teon001Geon001Aeo * RT * SC * ST * SG * ST * RA * | GTTGATCTGTA | RnXnXnXR SSSSR |
| 19838 | SG * SC * SA * SG * Rm5Ceo * SmA * SmG * SmC * SmU | GCAGCAGCU | SSSSR SSSS |
| WV- | Geo * RTeon001RTeon001RGeon001RAeo * RT * SC * ST * SG * ST * | GTTGATCTGTA | RnRnRnRR SSSSR |
| 19839 | RA * SG * SC * SA * SG * Rm5Ceo * SmA * SmG * SmC * SmU | GCAGCAGCU | SSSSR SSSS |
| WV- | Geo * RTeon001STeon001SGeon001SAeo * RT * SC * ST * SG * ST * | GTTGATCTGTA | RnSnSnSR SSSSR |
| 19840 | RA * SG * SC * SA * SG * Rm5Ceo * SmA * SmG * SmC * SmU | GCAGCAGCU | SSSSR SSSS |
| WV- | Geo * RTeon001Teon001Geon001Aeo * RT * SCn001T * SG * ST * RA * | GTTGATCTGTA | RnXnXnXR SnXSSR |
| 19841 | SG * SCn001A * SG * Rm5Ceo * SmA * SmG * SmC * SmU | GCAGCAGCU | SSnXSR SSSS |
| WV- | Geo * RTeon001Teon001Geon001Aeo * RT * SCn001T * SG * ST * RA * | GTTGATCTGTA | RnXnXnXR SnXSSR |
| 19842 | SG * SC * SA * SG * Rm5Ceo * SmA * SmG * SmC * SmU | GCAGCAGCU | SSSSRSSSS |
| WV- | Geo * RTeon001Teon001Geon001Aeo * RT * SC * ST * SG * ST * RA * | GTTGATCTGTA | RnXnXnXR SSSSR |
| 19843 | SG * SCn001A * SG * Rm5Ceo * SmA * SmG * SmC * SmU | GCAGCAGCU | SSnXSR SSSS |
| WV- | Geo * RTeon001RTeon001RGeon001RAeo * RT * SCn001RT * SG * ST | GTTGATCTGTA | RnRnRnRR SnRSSR |
| 19844 | * RA * SG * SCn001RA * SG * Rm5Ceo * SmA * SmG * SmC * SmU | GCAGCAGCU | SSnRSR SSSS |
| WV- | Geo * RTeon001RTeon001RGeon001RAeo * RT * SCn001RT * SG * ST | GTTGATCTGTA | RnRnRnRR SnRSSR |
| 19845 | * RA * SG * SC * SA * SG * Rm5Ceo * SmA * SmG * SmC * SmU | GCAGCAGCU | SSSSR SSSS |
| WV- | Geo * RTeon001RTeon001RGeon001RAeo * RT * SC * ST * SG * ST * | GTTGATCTGTA | RnRnRnRR SSSSR |
| 19846 | RA * SG * SCn001RA * SG * Rm5Ceo * SmA * SmG * SmC * SmU | GCAGCAGCU | SSnRSR SSSS |
| WV- | Geo * RTeon001RTeon001RGeon001RAeo * RT * SCn001ST * SG * ST | GTTGATCTGTA | RnRnRnRR SnSSSR |
| 19847 | * RA * SG * SCn001SA * SG * Rm5Ceo * SmA * SmG * SmC * SmU | GCAGCAGCU | SSnSSR SSSS |
| WV- | Geo * RTeon001RTeon001RGeon001RAeo * RT * SCn001ST * SG * ST | GTTGATCTGTA | RnRnRnRR SnSSSR |
| 19848 | * RA * SG * SC * SA * SG * Rm5Ceo * SmA * SmG * SmC * SmU | GCAGCAGCU | SSSSR SSSS |
| WV- | Geo * RTeon001RTeon001RGeon001RAeo * RT * SC * ST * SG * ST * | GTTGATCTGTA | RnRnRnRR SSSSR |
| 19849 | RA * SG * SCn001SA * SG * Rm5Ceo * SmA * SmG * SmC * SmU | GCAGCAGCU | SSnSSR SSSS |
| WV- | Geo * RTeon001STeon001SGeon001SAeo * RT * SCn001RT * SG * ST * | GTTGATCTGTA | RnSnSnSR SnRSSR |
| 19850 | RA * SG * SCn001RA * SG * Rm5Ceo * SmA * SmG * SmC * SmU | GCAGCAGCU | SSnRSR SSSS |
| WV- | Geo * RTeon001STeon001SGeon001SAeo * RT * SCn001RT * SG * ST * | GTTGATCTGTA | RnSnSnSR SnRSSR |
| 19851 | RA * SG * SC * SA * SG * Rm5Ceo * SmA * SmG * SmC * SmU | GCAGCAGCU | SSSSR SSSS |
| WV- | Geo * RTeon001STeon001SGeon001SAeo * RT * SC * ST * SG * ST * | GTTGATCTGTA | RnSnSnSR SSSSR |
| 19852 | RA * SG * SCn001RA * SG * Rm5Ceo * SmA * SmG * SmC * SmU | GCAGCAGCU | SSnRSR SSSS |
| WV- | Geo * RTeon001STeon001SGeon001SAe * RT * SCn001ST * SG * ST * | GTTGATCTGTA | RnSnSnSR SnSSSR |
| 19853 | RA * SG * SCn001SA * SG * Rm5Ceo * SmA * SmG * SmC * SmU | GCAGCAGCU | SSnSSR SSSS |
| WV- | Geo * RTeon001STeon001SGeon001SAeo * RT * SCn001ST * SG * ST * | GTTGATCTGTA | RnSnSnSR SnSSSR |
| 19854 | RA * SG * SC * SA * SG * Rm5Ceo * SmA * SmG * SmC * SmU | GCAGCAGCU | SSSSR SSSS |
| WV- | Geo * RTeon001STeon001SGeon001SAeo * RT * SC * ST * SG * ST * | GTTGATCTGTA | RnSnSnSR SSSSR |
| 19855 | RA * SG * SCn001SA * SG * Rm5Ceo * SmA * SmG * SmC * SmU | GCAGCAGCU | SSnSSR SSSS |
| WV- | mU * RmU * SmG * SmA * STeo * RC * ST * SG * ST * RA * SG | UUGATCTGTAGCAGCAGCTT | RSSSR SSSRS SSSSR |
| 19856 | * SC * SA * SG * SC * RAeo * SGeo * Sm5Ceo * STeo * RTeo | | SSSR |
| WV- | mU * RmU * SmG * SmA * SmU * SC * ST * SG * ST * RA * SG | UUGAUCTGTAGCAGCAGCTT | RSSSS SSSRS SSSSR |
| 19857 | * SC * SA * SG * SC * RAeo * SGeo * Sm5Ceo * STeo * RTeo | | SSSR |
| WV- | mU * SmU * SmG * SmA * STeo * RC * ST * SG * ST * RA * SG | UUGATCTGTAGCAGCAGCTT | SSSSR SSSRS SSSSR |
| 19858 | * SC * SA * SG * SC * RAeo * SGeo * Sm5Ceo * STeo * RTeo | | SSSR |
| WV- | mU * RmU * SmG * SmA * STeo * RC * ST * SG * ST * RA * SG | UUGATCTGTAGCAGCAGCTT | RSSSR SSSRS SSSSR |
| 19859 | * SC * SA * SG * SC * RAeo * SGeo * Sm5Ceo * STeo * STeo | | SSSS |
| WV- | mU * SmU * SmG * SmA * STeo * RC * ST * SG * ST * RA * SG | UUGATCTGTAGCAGCAGCTT | SSSSR SSSRS SSSSR |
| 19860 | * SC * SA * SG * SC * RAeo * SGeo * Sm5Ceo * STeo * STeo | | SSSS |
| WV- | mU * SmU * SmG * SmA * SmU * SC * ST * SG * ST * RA * SG | UUGAUCTGTAGCAGCAGCTT | SSSSS SSSRS SSSSR |
| 19861 | * SC * SA * SG * SC * RAeo * SGeo * Sm5Ceo * STeo * RTeo | | SSSR |
| WV- | mU * SmU * SmG * SmA * SmU * SC * ST * SG * ST * RA * SG | UUGAUCTGTAGCAGCAGCTT | SSSSS SSSRS SSSSS |
| 19862 | * SC * SA * SG * SC * SAeo * SGeo * Sm5Ceo * STeo * STeo | | SSSS |
| WV- | mU * RmU * SmG * SmA * STeo * RC * ST * SG * ST * RA * SG | UUGATCTGTAGCAGCAGCTT | RSSSR SSSRS SSSSS |
| 19863 | * SC * SA * SG * SC * SAeo * SGeo * Sm5Ceo * STeo * STeo | | SSSS |
| WV- | mU * RmU * SmG * SmA * STeo * RC * ST * SG * RT * SA * SG | UUGATCTGTAGCAGCAGCTT | RSSSR SSRSS SSSSR |
| 19864 | * SC * SA * SG * SC * RAeo * SGeo * Sm5Ceo * STeo * RTeo | | SSSR |
| WV- | mU * RmU * SmG * SmA * SmU * SC * ST * SG * RT * SA * SG | UUGAUCTGTAGCAGCAGCTT | RSSSS SSRSS SSSSR |
| 19865 | * SC * SA * SG * SC * RAeo * SGeo * Sm5Ceo * STeo * RTeo | | SSSR |
| WV- | mU * SmU * SmG * SmA * STeo * RC * ST * SG * RT * SA * SG | UUGATCTGTAGCAGCAGCTT | SSSSR SSRSS SSSSR |
| 19866 | * SC * SA * SG * SC * RAeo * SGeo * Sm5Ceo * STeo * RTeo | | SSSR |
| WV- | mU * RmU * SmG * SmA * STeo * RC * ST * SG * RT * SA * SG | UUGATCTGTAGCAGCAGCTT | RSSSR SSRSS SSSSR |
| 19867 | * SC * SA * SG * SC * RAeo * SGeo * Sm5Ceo * STeo * STeo | | SSSS |
| WV- | mU * SmU * SmG * SmA * STeo * RC * ST * SG * RT * SA * SG | UUGATCTGTAGCAGCAGCTT | SSSSR SSRSS SSSSR |
| 19868 | * SC * SA * SG * SC * RAeo * SGeo * Sm5Ceo * STeo * STeo | | SSSS |
| WV- | mU * SmU * SmG * SmA * SmU * SC * ST * SG * RT * SA * SG | UUGAUCTGTAGCAGCAGCTT | SSSSS SSRSS SSSSR |
| 19869 | * SC * SA * SG * SC * RAeo * SGeo * 5m5Ceo * STeo * RTeo | | SSSR |
| WV- | mU * SmU * SmG * SmA * SmU * SC * ST * SG * RT * SA * SG | UUGAUCTGTAGCAGCAGCTT | SSSSS SSRSS SSSSS |
| 19870 | * SC * SA * SG * SC * SAeo * SGeo * Sm5Ceo * STeo * STeo | | SSSS |
| WV- | mU * RmU * SmG * SmA * STeo * RC * ST * SG * RT * SA * SG | UUGATCTGTAGCAGCAGCTT | RSSSR SSRSS SSSSS |
| 19871 | * SC * SA * SG * SC * SAeo * SGeo * 5m5Ceo * STeo * STeo | | SSSS |
| WV- | mG * SGeon001m5Ceon001Aeon001mC * SA * SA * SG * SG * SG * | GGCACAAGGGC | SnXnXnXS SSSSS |
| 19872 | SC * SA * SC * RA * SG * SmA * SmC * SmU * SmU * SmC | ACAGACUUC | SSRSS SSSS |
| WV- | mG * Sm5Ceon001Aeon001m5Ceon001mA * SA * SG * SG * SG * SC * | GCACAAGGGCA | SnXnXnXS SSSSS |
| 19873 | SA * SC * SA * RG * SA * SmC * SmU * SmU * SmC * SmC | CAGACUUCC | SSRSS SSSS |
| WV- | mG * SGeon001m5Ceon001Aeon001mC * SA * SA * SG * SG * SG * | GGCACAAGGGC | SnXnXnXS SSSSS |
| 19874 | SC * SA * SC * SA * RG * SmA * SmC * SmU * SmU * SmC | ACAGACUUC | SSSRS SSSS |
| WV- | mG * Sm5Ceon001Aeon001m5Ceon001Aeo * RA * SG * SG * SG * SC | GCACAAGGGCA | SnXnXnXR SSSSS |
| 19875 | * SA * SC * RA * SG * SA * SmC * SmU * SmU * SmC * SmC | CAGACUUCC | SRSSS SSSS |
| WV- | mG * SGeon001m5Ceon001Aeon001m5Ceo * RA * SA * SG * SG * RG | GGCACAAGGGC | SnXnXnXR SSSRS |
| 19876 | * SC * SA * SC * SA * SG * SmA * SmC * SmU * SmU * SmC | ACAGACUUC | SSSSS SSSS |
| WV- | mG * SGeon001m5Ceon001Aeon001m5Ceo * RA * SA * SG * SG * SG | GGCACAAGGGC | SnXnXnXR SSSSR |
| 19877 | * RC * SA * SC * SA * SG * SmA * SmC * SmU * SmU * SmC | ACAGACUUC | SSSSS SSSS |
| WV- | mG * Sm5Ceon001Aeon001m5Ceon001mA * SA * SG * SG * SG * SC * | GCACAAGGGCA | SnXnXnXS SSSSS |
| 19878 | SA * SC * RA * SG * SA * Rm5Ceo * SmU * SmU * SmC * SmC | CAGACUUCC | SRSSR SSSS |
| WV- | mG * SGeon001m5Ceon001Aeon001mC * SA * SA * SG * SG * RG * | GGCACAAGGGC | SnXnXnXS SSSRS |
| 19879 | SC * SA * SC * SA * SG * RAeo * SmC * SmU * SmU * SmC | ACAGACUUC | SSSSR SSSS |
| WV- | mG * SGeon001m5Ceon001Aeon001mC * SA * SA * SG * SG * SG * | GGCACAAGGGC | SnXnXnXS SSSSR |
| 19880 | RC * SA * SC * SA * SG * RAeo * SmC * SmU * SmU * SmC | ACAGACUUC | SSSSR SSSS |
| WV- | mG * Sm5Ceon001Aeon001m5Ceon001Aeo * RA * SG * SG * SG * SC | GCACAAGGGCA | SnXnXnXR SSSSS |
| 19881 | * SA * SC * RA * SG * SA * Rm5Ceo * SmU * SmU * SmC * SmC | CAGACUUCC | SRSSR SSSS |
| WV- | mG * SGeon001m5Ceon001Aeon001m5Ceo * RA * SA * SG * SG * RG | GGCACAAGGGC | SnXnXnXR SSSRS |
| 19882 | * SC * SA * SC * SA * SG * RAeo * SmC * SmU * SmU * SmC | ACAGACUUC | SSSSR SSSS |
| WV- | mG * SGeon001m5Ceon001Aeon001m5Ceo * RA * SA * SG * SG * SG | GGCACAAGGGC | SnXnXnXR SSSSR |
| 19883 | * RC * SA * SC * SA * SG * RAeo * SmC * SmU * SmU * SmC | ACAGACUUC | SSSS SSSS |
| WV- | Mod039L001Geo * SAeom5Ceom5CeoTeo * RC * ST * SG * RT * | GACCTCTGTGAAAGCCAACA | OSOOO RSSRS |
| 20213 | SG * SA * SA * SA * RG * SC * Sm5CeoAeoAeom5Ceo * SAeo | | SSSRS SOOOS |
| WV- | mAmC * SA * SA * SG * SG * SG * SC * SA * SC * RA * SG * | ACAAGGGCACAGACUUC | OSSSSSSSSRSSOOOS |
| 20306 | SmAmCmUmU * SmC | | |
| WV- | mG * SmGmCmAmC * SA * SA * SG * SG * SG * SC * SA * SC | GGCACAAGGGC ACAGACUU | SOOOS SSSSS SSRSS |
| 20307 | * RA * SG * SmAmCmUmU | | OOO |
| WV- | mG * SmGmCmAmC * SA * SA * SG * SG * SG * SC * SA * SC | GGCACAAGGGC ACAGACU | SOOOS SSSSS SSRSS |
| 20308 | * RA * SG * SmAmCmU | | OO |
| WV- | mG * SmGmCmAmC * SA * SA * SG * SG * SG * SC * SA * SC | GGCACAAGGGC ACAGAC | SOOOS SSSSS SSRSS |
| 20309 | * RA * SG * SmAmC | | O |
| WV- | mCmA * SC * SA * SC * SA * SG * ST * SA * SG * RA * ST * | CACACAGTAGATGAGGG | OSSSS SSSSR |
| 20310 | SmGmAmGmG * SmG | | SSOOOS |
| WV- | mG * SmUmGmCmA * SC * SA * SC * SA * SG * ST * SA * SG | GUGCACACAGTAGATGAGG | SOOOS SSSSS SSRSS |
| 20311 | * RA * ST * SmGmAmGmG | | OOO |
| WV- | mG * SmUmGmCmA * SC * SA * SC * SA * SG * ST * SA * SG | GUGCACACAGTAGATGAG | SOOOS SSSSS SSRSS |
| 20312 | * RA * ST * SmGmAmG | | OO |
| WV- | mG * SmUmGmCmA * SC * SA * SC * SA * SG * ST * SA * SG | GUGCACACAGTAGATGA | SOOOS SSSSS SSRSS |
| 20313 | * RA * ST * SmGmA | | O |
| WV- | G * SmGmCmAmC * SA * SA * SG * SG * SG * SC * SA * SC * | GGCACAAGGGC ACAGACUUC | SOOOS SSSSS SSSSS |
| 21175 | SA * SG * SmAmCmUmU * SC | | OOOS |
| WV- | mA * Sm5Ceom5CeoGeomC * RC * SA * ST * SC * RC * SC * | ACCGCCATCCCCGCCGUAGC | SOOOR SSSRS |
| 21178 | Sm5C * RG * SC * Sm5C * RmG * SmU * SmA * SmG * SmC | | SRSSR SSSS |
| WV- | mG * STeoTeoAeomC * Rm5C * SG * RC * SC * RA * ST * SC * | GTTACCGCCATCCCCGCCGU | SOOOR SRSRS |
| 21179 | SC * SC * Sm5C * RmG * SmC * SmC * SmG * SmU | | SSSSR SSSS |
| WV- | mG * STeoTeoAeomC * Sm5C * SG * RC * SC * RA * ST * SC * | GTTACCGCCATCCCCGCCGU | SOOOS SRSRS SSSSR |
| 21180 | SC * SC * Sm5C * RmG * SmC * SmC * SmG * SmU | | SSSS |
| WV- | mA * Sm5Ceom5CeoGeomC * SC * SA * ST * SC * RC * SC * | ACCGCCATCCCCGCCGUAGC | SOOOS SSSRS SRSSR |
| 21181 | Sm5C * RG * SC * Sm5C * RmG * SmU * SmA * SmG * SmC | | SSSS |
| WV- | mA * Sm5Ceom5CeoGeomC * SG * RA * SC * SC * SC * ST * | ACCGCGACCCTCTGGACAGG | SOOOS RSSSS SSSSR |
| 21182 | SC * ST * SG * SG * RmA * SmC * SmA * SmG * SmG | | SSSS |
| WV- | mA * Sm5Ceom5CeoGeomC * SG * SA * RC * SC * SC * ST * | ACCGCGACCCTCTGGACAGG | SOOOS SRSSS SSSSR |
| 21183 | SC * ST * SG * SG * RmA * SmC * SmA * SmG * SmG | | SSSS |
| WV- | mA * Sm5Ceom5CeoGeomC * SG * SA * SC * RC * SC * ST * | ACCGCGACCCTCTGGACAGG | SOOOS SSRSS SSSSR |
| 21184 | SC * ST * SG * SG * RmA * SmC * SmA * SmG * SmG | | SSSS |
| WV- | mA * Sm5Ceom5CeoGeomC * SG * SA * SC * SC * RC * ST * | ACCGCGACCCTCTGGACAGG | SOOOS SSSRS SSSSR |
| 21185 | SC * ST * SG * SG * RmA * SmC * SmA * SmG * SmG | | SSSS |
| WV- | mA * Sm5Ceom5CeoGeomC * SG * SA * SC * SC * SC * RT * | ACCGCGACCCTCTGGACAGG | SOOOS SSSSR SSSSR |
| 21186 | SC * ST * SG * SG * RmA * SmC * SmA * SmG * SmG | | SSSS |
| WV- | mA * Sm5Ceom5CeoGeomC * SG * SA * SC * SC * SC * ST * | ACCGCGACCCTCTGGACAGG | SOOOS SSSSS RSSSR |
| 21187 | RC * ST * SG * SG * RmA * SmC * SmA * SmG * SmG | | SSSS |
| WV- | mA * Sm5Ceom5CeoGeomC * SG * SA * SC * SC * SC * ST * SC | ACCGCGACCCTCTGGACAGG | SOOOS SSSSS SRSSR |
| 21188 | * RT * SG * SG * RmA * SmC * SmA * SmG * SmG | | SSSS |
| WV- | mA * Sm5Ceom5CeoGeomC * SG * SA * SC * SC * SC * ST * | ACCGCGACCCTCTGGACAGG | SOOOS SSSSS SSRSR |
| 21189 | SC * ST * RG * SG * RmA * SmC * SmA * SmG * SmG | | SSSS |
| WV- | mC * SAeoGeom5CeomA * Sm5C * RG * SC * SA * SG * SG * | CAGCACGCAGGCCAGGGGCG | SOOOS RSSSS SSSSR |
| 21190 | SC * SC * SA * SG * RmG * SmG * SmG * SmC * SmG | | SSSS |
| WV- | mC * SAeoGeom5CeomA * Sm5C * SG * RC * SA * SG * SG * | CAGCACGCAGGCCAGGGGCG | SOOOS SRSSS SSSSR |
| 21191 | SC * SC * SA * SG * RmG * SmG * SmG * SmC * SmG | | SSSS |
| WV- | mC * SAeoGeom5CeomA * Sm5C * SG * SC * RA * SG * SG * | CAGCACGCAGGCCAGGGGCG | SOOOS SSRSS SSSSR |
| 21192 | SC * SC * SA * SG * RmG * SmG * SmG * SmC * SmG | | SSSS |
| WV- | mC * SAeoGeom5CeomA * Sm5C * SG * SC * SA * RG * SG * | CAGCACGCAGGCCAGGGGCG | SOOOS SSSRS SSSSR |
| 21193 | SC * SC * SA * SG * RmG * SmG * SmG * SmC * SmG | | SSSS |
| WV- | mC * SAeoGeom5CeomA * Sm5C * SG * SC * SA * SG * RG * | CAGCACGCAGGCCAGGGGCG | SOOOS SSSSR SSSSR |
| 21194 | SC * SC * SA * SG * RmG * SmG * SmG * SmC * SmG | | SSSS |
| WV- | mC * SAeoGeom5CeomA * Sm5C * SG * SC * SA * SG * SG * | CAGCACGCAGGCCAGGGGCG | SOOOS SSSSS RSSSR |
| 21195 | RC * SC * SA * SG * RmG * SmG * SmG * SmC * SmG | | SSSS |
| WV- | mC * SAeoGeom5CeomA * Sm5C * SG * SC * SA * SG * SG * | CAGCACGCAGGCCAGGGGCG | SOOOS SSSSS SRSSR |
| 21196 | SC * RC * SA * SG * RmG * SmG * SmG * SmC * SmG | | SSSS |
| WV- | mC * SAeoGeom5CeomA * Sm5C * SG * SC * SA * SG * SG * | CAGCACGCAGGCCAGGGGCG | SOOOS SSSSS SSRSS |
| 21197 | SC * SC * RA * SG * SmG * SmG * SmG * SmC * SmG | | SSSS |
| WV- | mU * SGeom5Ceom5CeomC * SA * RG * SC * SA * Sm5C * SG | UGCCCAGCACGCAGGCCAGG | SOOOS RSSSS SSSSR |
| 21198 | * SC * SA * SG * SG * RmC * SmC * SmA * SmG * SmG | | SSSS |
| WV- | mU * SGeom5Ceom5CeomC * SA * SG * RC * SA * Sm5C * SG | UGCCCAGCACGCAGGCCAGG | SOOOS SRSSS SSSSR |
| 21199 | * SC * SA * SG * SG * RmC * SmC * SmA * SmG * SmG | | SSSS |
| WV- | mU * SGeom5Ceom5CeomC * SA * SG * SC * RA * Sm5C * SG | UGCCCAGCACGCAGGCCAGG | SOOOS SSRSS SSSSR |
| 21200 | * SC * SA * SG * SG * RmC * SmC * SmA * SmG * SmG | | SSSS |
| WV- | mU * SGeom5Ceom5CeomC * SA * SG * SC * SA * Rm5C * SG | UGCCCAGCACGCAGGCCAGG | SOOOS SSSRS SSSSR |
| 21201 | * SC * SA * SG * SG * RmC * SmC * SmA * SmG * SmG | | SSSS |
| WV- | mU * SGeom5Ceom5CeomC * SA * SG * SC * SA * Sm5C * RG | UGCCCAGCACGCAGGCCAGG | SOOOS SSSSR SSSSR |
| 21202 | * SC * SA * SG * SG * RmC * SmC * SmA * SmG * SmG | | SSSS |
| WV- | mU * SGeom5Ceom5CeomC * SA * SG * SC * SA * Sm5C * SG | UGCCCAGCACGCAGGCCAGG | SOOOS SSSSS RSSSR |
| 21203 | * RC * SA * SG * SG * RmC * SmC * SmA * SmG * SmG | | SSSS |
| WV- | mU * SGeom5Ceom5CeomC * SA * SG * SC * SA * Sm5C * SG | UGCCCAGCACGCAGGCCAGG | SOOOS SSSSS SRSSR |
| 21204 | * SC * RA * SG * SG * RmC * SmC * SmA * SmG * SmG | | SSSS |
| WV- | mU * SGeom5Ceom5CeomC * SA * SG * SC * SA * Sm5C * SG | UGCCCAGCACGCAGGCCAGG | SOOOS SSSSS SSRSS |
| 21205 | * SC * SA * RG * SG * SmC * SmC * SmA * SmG * SmG | | SSSS |
| WV- | mG * S mU * S mU * S mG * S mA * S mU * SC * ST * SG * ST * RA * | GUUGAUCTGTA | SSSSS SSSSR SSSSR |
| 21260 | SG * SC * SA * SG * R m5CeoAeoGeo m5Ceo * STeo | GCAGCAGCT | OOOS |
| WV- | mG * S mU * S mU * S mG * S mA * S mU * S mC * ST * SG * ST * RA * | GUUGAUCTGTA | SSSSS SSSSR SSSSR |
| 21261 | SG * SC * SA * SG * R m5CeoAeoGeo m5Ceo * STeo | GCAGCAGCT | OOOS |
| WV- | mG * S mU * S mU * S mG * S mA * ST * SC * ST * SG * ST * RA * SG * | GUUGATCTGTA | SSSSS SSSSR SSSRO |
| 21262 | SC * SA * RGeo m5CeoAeoGeo m5Ceo * STeo | GCAGCAGCT | OOOS |
| WV- | mG * S mU * S mU * S mG * S mA * ST * SC * ST * SG * ST * RA * SG * | GUUGATCTGTA | SSSSS SSSSR SSRO |
| 21263 | SC * RAeoGeo m5CeoAeoGeo m5Ceo * STeo | GCAGCAGCT | OOOOS |
| WV- | mG * S mU * S mU * S mG * S mA * S mU * S mC * ST * SG * ST * RA * | GUUGAUCTGTA | SSSSS SSSSR SSSRO |
| 21264 | SG * SC * SA * RGeo m5CeoAeoGeo m5Ceo * STeo | GCAGCAGCT | OOOS |
| WV- | mG * S mU * S mU * S mG * S mA * S mU * SC * ST * SG * ST * RA * | GUUGAUCTGTA | SSSSS SSSSR SSRO |
| 21265 | SG * SC * RAeoGeo m5CeoAeoGeo m5Ceo * STeo | GCAGCAGCT | OOOOS |
| WV- | mG * S mU * S mU * S mG * S mA * S mU * S mC * ST * SG * ST * RA * | GUUGAUCTGTA | SSSSS SSSSR SSRO |
| 21266 | SG * SC * RAeoGeo m5CeoAeoGeo m5Ceo * STeo | GCAGCAGCT | OOOOS |
| WV- | mG * S mU * S mU * S mG * S mA * S mU * SC * ST * SG * ST * RA * | GUUGAUCTGTA | SSSSS SSSSR SSSSR |
| 21267 | SG * SC * SA * SG * R m5Ceon001Aeon001Geon001 m5Ceo * STeo | GCAGCAGCT | nXnXnXS |
| WV- | mG * S mU * S mU * S mG * S mA * S mU * S mC * ST * SG * ST * RA * | GUUGAUCTGTA | SSSSS SSSSR SSSSR |
| 21268 | SG * SC * SA * SG * R m5Ceon001Aeon001Geon001 m5Ceo * STeo | GCAGCAGCT | nXnXnXS |
| WV- | mG * S mU * S mU * S mG * S mA * ST * SC * ST * SG * ST * RA * SG * | GUUGATCTGTA | SSSSS SSSSR SSSR |
| 21269 | SC * SA * RGeon001 m5Ceon001Aeon001Geon001 m5Ceo * STeo | GCAGCAGCT | nXnX nXnXS |
| WV- | mG * S mU * S mU * S mG * S mA * ST * SC * ST * SG * ST * RA * SG * | GUUGATCTGTA | SSSSS SSSSR SSR |
| 21270 | SC * RAeon001Geon001 m5Ceon001Aeon001Geon001 m5Ceo * STeo | GCAGCAGCT | nXnX nXnXnXS |
| WV- | mG * S mU * S mU * S mG * S mA * S mU * S mC * ST * SG * ST * RA * | GUUGAUCTGTA | SSSSS SSSSR SSSR |
| 21271 | SG * SC * SA * RGeon001 m5Ceon001Aeon001Geon001 m5Ceo * STeo | GCAGCAGCT | nXnX nXnXS |
| WV- | mG * S mU * S mU * S mG * S mA * S mU * SC * ST * SG * ST * RA * | GUUGAUCTGTA | SSSSS SSSSR SSR |
| 21272 | SG * SC * RAeon001Geon001 m5Ceon001Aeon001Geon001 m5Ceo * STeo | GCAGCAGCT | nXnX nXnXnXS |
| WV- | mG * S mU * S mU * S mG * S mA * S mU * S mC * ST * SG * ST * RA * | GUUGAUCTGTA | SSSSS SSSSR SSR |
| 21273 | SG * SC * RAeon001Geon001 m5Ceon001Aeon001Geon001 m5Ceo * STeo | GCAGCAGCT | nXnX nXnXnXS |
| WV- | mG * S mUn001 mU * S mGn001 mA * S mU * SC * ST * SG * ST * RA * | GUUGAUCTGTA | SnXSnX SSSSS RSSSSR |
| 21274 | SG * SC * SA * SG * R m5Ceon001AeoGeon001 m5Ceo * STeo | GCAGCAGCT | nXOnXS |
| WV- | mG * S mUn001 mU * S mGn001 mA * S mU * S mC * ST * SG * ST * RA | GUUGAUCTGTA | SnXSnX SSSSS RSSSSR |
| 21275 | * SG * SC * SA * SG * R m5Ceon001AeoGeon001 m5Ceo * STeo | GCAGCAGCT | nXOnXS |
| WV- | mG * S mUn001 mU * S mGn001 mA * S mUn001 mC * ST * SG * ST * | GUUGAUCTGTA | SnXSnXS nXSSSR |
| 21276 | RA * SG * SC * SA * SG * R m5Ceon001AeoGeon001 m5Ceo * STeo | GCAGCAGCT | SSSSR nXOnXS |
| WV- | mG * S mUn001 mU * S mGn001 mA * ST * SC * ST * SG * ST * RA * SG | GUUGATCTGTA | SnXSnX SSSSS RSSSRO |
| 21277 | * SC * SA * RGeo m5Ceon001AeoGeon001 m5Ceo * STeo | GCAGCAGCT | nXOnXS |
| WV- | mG * S mUn001 mU * S mGn001 mA * ST * SC * ST * SG * ST * RA * SG | GUUGATCTGTA | SnXSnX SSSSS RSSRO |
| 21278 | * SC * RAeoGeo m5Ceon001AeoGeon001 m5Ceo * STeo | GCAGCAGCT | OnXOnXS |
| WV- | mG * S mUn001 mU * S mGn001 mA * ST * SC * ST * SG * ST * RA * SG | GUUGATCTGTA | SnXSnX SSSSS |
| 21279 | * SC * RAeon001Geo m5Ceon001AeoGeon001 m5Ceo * STeo | GCAGCAGCT | RSSRnXOnXOnXS |
| WV- | mG * S mUn001 mU * S mGn001 mA * S mU * S mC * ST * SG * ST * RA | GUUGAUCTGTA | SnXSnX SSSSS RSSSRO |
| 21280 | * SG * SC * SA * RGeo m5Ceon001AeoGeon001 m5Ceo * STeo | GCAGCAGCT | nXOnXS |
| WV- | mG * S mUn001 mU * S mGn001 mA * S mUn001 mC * ST * SG * ST * | GUUGAUCTGTA | SnXSnXSnX SSSRS |
| 21281 | RA * SG * SC * SA * RGeo m5Ceon001AeoGeon001 m5Ceo * STeo | GCAGCAGCT | SSRO nXOnXS |
| WV- | mG * S mUn001 mU * S mGn001 mA * S mU * SC * ST * SG * ST * RA * | GUUGAUCTGTA | SnXSnX SSSSS RSSRO |
| 21282 | SG * SC * RAeoGeo m5Ceon001AeoGeon001 m5Ceo * STeo | GCAGCAGCT | OnXOnXS |
| WV- | mG * S mUn001 mU * S mGn001 mA * S mU * SC * ST * SG * ST * RA * | GUUGAUCTGTA | SnXSnX SSSSS |
| 21283 | SG * SC * RAeon001Geo m5Ceon001AeoGeon001 m5Ceo * STeo | GCAGCAGCT | RSSRnXOnXOnXS |
| WV- | mG * S mUn001 mU * S mGn001 mA * S mU * S mC * ST * SG * ST * RA | GUUGAUCTGTA | SnXSnX SSSSS RSSRO |
| 21284 | * SG * SC * RAeoGeo m5Ceon001AeoGeon001 m5Ceo * STeo | GCAGCAGCT | OnXOnXS |
| WV- | mG * S mUn001 mU * S mGn001 mA * S mUn001 mC * ST * SG * ST * | GUUGAUCTGTA | SnXSnXSnXSSSRSSRnX |
| 21285 | RA * SG * SC * RAeon001Geo m5Ceon001AeoGeon001 m5Ceo * STeo | GCAGCAGCT | OnXOnXS |
| WV- | mG * S mU * S mU * S mG * S mA * ST * ST * ST * SG * ST * RA * SG * | GUUGATTTGTA | SSSSS SSSSR SSSSR |
| 21286 | SC * SA * SG * R m5CeoAeoGeo m5Ceo * STeo | GCAGCAGCT | OOOS |
| WV- | mG * S mU * S mU * S mG * S mA * ST * SC * ST * SG * ST * RA * SG * | GUUGATCTGTA | SSSSS SSSSR SSSSR |
| 21287 | ST * SA * SG * R m5CeoAeoGeo m5Ceo * STeo | GTAGCAGCT | OOOS |
| WV- | mG * S mU * S mU * S mG * S mA * S mU * ST * ST * SG * ST * RA * SG | GUUGAUTTGTA | SSSSS SSSSR SSSSR |
| 21288 | * SC * SA * SG * R m5CeoAeoGeo m5Ceo * STeo | GCAGCAGCT | OOOS |
| WV- | mG * S mU * S mU * S mG * S mA * S mU * SC * ST * SG * ST * RA * | GUUGAUCTGTA | SSSSS SSSSR SSSSR |
| 21289 | SG * ST * SA * SG * R m5CeoAeoGeo m5Ceo * STeo | GTAGCAGCT | OOOS |
| WV- | mG * S mU * S mU * S mG * S mA * S mU * S mU * ST * SG * ST * RA * | GUUGAUUTGTA | SSSSS SSSSR SSSSR |
| 21290 | SG * SC * SA * SG * R m5CeoAeoGeo m5Ceo * STeo | GCAGCAGCT | OOOS |
| WV- | mG * S mU * S mU * S mG * S mA * S mU * S mC * ST * SG * ST * RA * | GUUGAUCTGTA | SSSSS SSSSR SSSSR |
| 21291 | SG * ST * SA * SG * R m5CeoAeoGeo m5Ceo * STeo | GTAGCAGCT | OOOS |
| WV- | mG * S mU * S mU * S mG * S mA * ST * ST * ST * SG * ST * RA * SG * | GUUGATTTGTA | SSSSS SSSSR SSSRO |
| 21292 | SC * SA * RGeo m5CeoAeoGeo m5Ceo * STeo | GCAGCAGCT | OOOS |
| WV- | mG * S mU * S mU * S mG * S mA * ST * SC * ST * SG * ST * RA * SG * | GUUGATCTGTA | SSSSS SSSSR SSSRO |
| 21293 | ST * SA * RGeo m5CeoAeoGeo m5Ceo * STeo | GTAGCAGCT | OOOS |
| WV- | mG * S mU * S mU * S mG * S mA * ST * ST * ST * SG * ST * RA * SG * | GUUGATTTGTA | SSSSS SSSSR SSRO |
| 21294 | SC * RAeoGeo m5CeoAeoGeo m5Ceo * STeo | GCAGCAGCT | OOOOS |
| WV- | mG * S mU * S mU * S mG * S mA * ST * SC * ST * SG * ST * RA * SG * | GUUGATCTGTA | SSSSS SSSSR SSRO |
| 21295 | ST * RAeoGeo m5CeoAeoGeo m5Ceo * STeo | GTAGCAGCT | OOOOS |
| WV- | mG * S mU * S mU * S mG * S mA * S mU * S mU * ST * SG * ST * RA * | GUUGAUUTGTA | SSSSS SSSSR SSSRO |
| 21296 | SG * SC * SA * RGeo m5CeoAeoGeo m5Ceo * STeo | GCAGCAGCT | OOOS |
| WV- | mG * S mU * S mU * S mG * S mA * S mU * S mC * ST * SG * ST * RA * | GUUGAUCTGTA | SSSSS SSSSR SSSRO |
| 21297 | SG * ST * SA * RGeo m5CeoAeoGeo m5Ceo * STeo | GTAGCAGCT | OOOS |
| WV- | mG * S mU * S mU * S mG * S mA * S mU * ST * ST * SG * ST * RA * SG | GUUGAUTTGTA | SSSSS SSSSR SSRO |
| 21298 | * SC * RAeoGeo m5CeoAeoGeo m5Ceo * STeo | GCAGCAGCT | OOOOS |
| WV- | mG * S mU * S mU * S mG * S mA * S mU * SC * ST * SG * ST * RA * | GUUGAUCTGTA | SSSSS SSSSR SSRO |
| 21299 | SG * ST * RAeoGeo m5CeoAeoGeo m5Ceo * STeo | GTAGCAGCT | OOOOS |
| WV- | mG * S mU * S mU * S mG * S mA * S mU * S mU * ST * SG * ST * RA * | GUUGAUUTGTA | SSSSS SSSSR SSRO |
| 21300 | SG * SC * RAeoGeo m5CeoAeoGeo m5Ceo * STeo | GCAGCAGCT | OOOOS |
| WV- | mG * S mU * S mU * S mG * S mA * S mU * S mC * ST * SG * ST * RA * | GUUGAUCTGTA | SSSSS SSSSR SSRO |
| 21301 | SG * ST * RAeoGeo m5CeoAeoGeo m5Ceo * STeo | GTAGCAGCT | OOOOS |
| WV- | mG * S mU * S mU * S mG * S mA * ST * ST * ST * SG * ST * RA * SG * | GUUGATTTGTA | SSSSS SSSSR SSSSR |
| 21302 | SC * SA * SG * R m5Ceon001Aeon001Geon001 m5Ceo * STeo | GCAGCAGCT | nXnXnXS |
| WV- | mG * S mU * S mU * S mG * S mA * ST * SC * ST * SG * ST * RA * SG * | GUUGATCTGTA | SSSSS SSSSR SSSSR |
| 21303 | ST * SA * SG * R m5Ceon001Aeon001Geon001 m5Ceo * STeo | GTAGCAGCT | nXnXnXS |
| WV- | mG * S mU * S mU * S mG * S mA * S mU * ST * ST * SG * ST * RA * SG | GUUGAUTTGTA | SSSSS SSSSR SSSSR |
| 21304 | * SC * SA * SG * R m5Ceon001Aeon001Geon001 m5Ceo * STeo | GCAGCAGCT | nXnXnXS |
| WV- | mG * S mU * S mU * S mG * S mA * S mU * SC * ST * SG * ST * RA * | GUUGAUCTGTA | SSSSS SSSSR SSSSR |
| 21305 | SG * ST * SA * SG * R m5Ceon001Aeon001Geon001 m5Ceo * STeo | GTAGCAGCT | nXnXnXS |
| WV- | mG * S mU * S mU * S mG * S mA * S mU * S mU * ST * SG * ST * RA * | GUUGAUUTGTA | SSSSS SSSSR SSSSR |
| 21306 | SG * SC * SA * SG * R m5Ceon001Aeon001Geon001 m5Ceo * STeo | GCAGCAGCT | nXnXnXS |
| WV- | mG * S mU * S mU * S mG * S mA * S mU * S mC * ST * SG * ST * RA * | GUUGAUCTGTA | SSSSS SSSSR SSSSR |
| 21307 | SG * ST * SA * SG * R m5Ceon001Aeon001Geon001 m5Ceo * STeo | GTAGCAGCT | nXnXnXS |
| WV- | mG * S mU * S mU * S mG * S mA * ST * ST * ST * SG * ST * RA * SG * | GUUGATTTGTA | SSSSS SSSSR SSSR |
| 21308 | SC * SA * RGeon001 m5Ceon001Aeon001Geon001 m5Ceo * STeo | GCAGCAGCT | nXnX nXnXS |
| WV- | mG * S mU * S mU * S mG * S mA * ST * SC * ST * SG * ST * RA * SG * | GUUGATCTGTA | SSSSS SSSSR SSSR |
| 21309 | ST * SA * RGeon001 m5Ceon001Aeon001Geon001 m5Ceo * STeo | GTAGCAGCT | nXnX nXnXS |
| WV- | mG * S mU * S mU * S mG * S mA * ST * ST * ST * SG * ST * RA * SG * | GUUGATTTGTA | SSSSS SSSSR SSR |
| 21310 | SC * RAeon001Geon001 m5Ceon001Aeon001Geon001 m5Ceo * STeo | GCAGCAGCT | nXnX nXnXnXS |
| WV- | mG * S mU * S mU * S mG * S mA * ST * SC * ST * SG * ST * RA * SG * | GUUGATCTGTA | SSSSS SSSSR SSR |
| 21311 | ST * RAeon001Geon001 m5Ceon001Aeon001Geon001 m5Ceo * STeo | GTAGCAGCT | nXnX nXnXnXS |
| WV- | mG * S mU * S mU * S mG * S mA * S mU * S mU * ST * SG * ST * RA * | GUUGAUUTGTA | SSSSS SSSSR SSSR |
| 21312 | SG * SC * SA * RGeon001 m5Ceon001Aeon001Geon001 m5Ceo * STeo | GCAGCAGCT | nXnX nXnXS |
| WV- | mG * S mU * S mU * S mG * S mA * S mU * S mC * ST * SG * ST * RA * | GUUGAUCTGTA | SSSSS SSSSR SSSR |
| 21313 | SG * ST * SA * RGeon001 m5Ceon001Aeon001Geon001 m5Ceo * STeo | GTAGCAGCT | nXnX nXnXS |
| WV- | mG * S mU * S mU * S mG * S mA * S mU * ST * ST * SG * ST * RA * SG | GUUGAUTTGTA | SSSSS SSSSR SSR |
| 21314 | * SC * RAeon001Geon001 m5Ceon001Aeon001Geon001 m5Ceo * STeo | GCAGCAGCT | nXnX nXnXnXS |
| WV- | mG * S mU * S mU * S mG * S mA * S mU * SC * ST * SG * ST * RA * | GUUGAUCTGTA | SSSSS SSSSR SSR |
| 21315 | SG * ST * RAeon001Geon001 m5Ceon001Aeon001Geon001 m5Ceo * STeo | GTAGCAGCT | nXnX nXnXnXS |
| WV- | mG * S mU * S mU * S mG * S mA * S mU * S mU * ST * SG * ST * RA * | GUUGAUUTGTA | SSSSS SSSSR SSR |
| 21316 | SG * SC * RAeon001Geon001 m5Ceon001Aeon001Geon001 m5Ceo * STeo | GCAGCAGCT | nXnX nXnXnXS |
| WV- | mG * S mU * S mU * S mG * S mA * S mU * S mC * ST * SG * ST * RA * | GUUGAUCTGTA | SSSSS SSSSR SSR |
| 21317 | SG * ST * RAeon001Geon001 m5Ceon001Aeon001Geon001 m5Ceo * STeo | GTAGCAGCT | nXnX nXnXnXS |
| WV- | mG * S mUn001 mU * S mGn001 mA * ST * ST * ST * SG * ST * RA * SG | GUUGATTTGTA | SnXSnX SSSSS RSSSSR |
| 21318 | * SC * SA * SG * R m5Ceon001AeoGeon001 m5Ceo * STeo | GCAGCAGCT | nXOnXS |
| WV- | mG * S mUn001 mU * S mGn001 mA * ST * SC * ST * SG * ST * RA * SG | GUUGATCTGTA | SnXSnX SSSSS RSSSSR |
| 21319 | * ST * SA * SG * R m5Ceon001AeoGeon001 m5Ceo * STeo | GTAGCAGCT | nXOnXS |
| WV- | mG * S mUn001 mU * S mGn001 mA * S mU * ST * ST * SG * ST * RA * | GUUGAUTTGTA | SnXSnX SSSSS RSSSSR |
| 21320 | SG * SC * SA * SG * R m5Ceon001AeoGeon001 m5Ceo * STeo | GCAGCAGCT | nXOnXS |
| WV- | mG * S mUn001 mU * S mGn001 mA * S mU * SC * ST * SG * ST * RA * | GUUGAUCTGTA | SnXSnX SSSSS RSSSSR |
| 21321 | SG * ST * SA * SG * R m5Ceon001AeoGeon001 m5Ceo * STeo | GTAGCAGCT | nXOnXS |
| WV- | mG * S mUn001 mU * S mGn001 mA * S mU * S mU * ST * SG * ST * RA | GUUGAUUTGTA | SnXSnX SSSSS RSSSSR |
| 21322 | * SG * SC * SA * SG * R m5Ceon001AeoGeon001 m5Ceo * STeo | GCAGCAGCT | nXOnXS |
| WV- | mG * S mUn001 mU * S mGn001 mA * S mU * S mC * ST * SG * ST * RA | GUUGAUCTGTA | SnXSnX SSSSS RSSSSR |
| 21323 | * SG * ST * SA * SG * R m5Ceon001AeoGeon001 m5Ceo * STeo | GTAGCAGCT | nXOnXS |
| WV- | mG * S mUn001 mU * S mGn001 mA * S mUn001 mU * ST * SG * ST * | GUUGAUUTGTA | SnXSnXSnX SSSR |
| 21324 | RA * SG * SC * SA * SG * R m5Ceon001AeoGeon001 m5Ceo * STeo | GCAGCAGCT | SSSSR nXOnXS |
| WV- | mG * S mUn001 mU * S mGn001 mA * S mUn001 mC * ST * SG * ST * | GUUGAUCTGTA | SnXSnXSnX SSSR |
| 21325 | RA * SG * ST * SA * SG * R m5Ceon001AeoGeon001 m5Ceo * STeo | GTAGCAGCT | SSSSR nXOnXS |
| WV- | mG * S mUn001 mU * S mGn001 mA * ST * ST * ST * SG * ST * RA * SG | GUUGATTTGTA | SnXSnX SSSSS RSSSRO |
| 21326 | * SC * SA * RGeo m5Ceon001AeoGeon001 m5Ceo * STeo | GCAGCAGCT | nXOnXS |
| WV- | mG * S mUn001 mU * S mGn001 mA * ST * SC * ST * SG * ST * RA * SG | GUUGATCTGTA | SnXSnX SSSSS RSSSRO |
| 21327 | * ST * SA * RGeo m5Ceon001AeoGeon001 m5Ceo * STeo | GTAGCAGCT | nXOnXS |
| WV- | mG * S mUn001 mU * S mGn001 mA * ST * ST * ST * SG * ST * RA * SG | GUUGATTTGTA | SnXSnX SSSSS RSSRO |
| 21328 | * SC * RAeoGeo m5Ceon001AeoGeon001 m5Ceo * STeo | GCAGCAGCT | OnXOnXS |
| WV- | mG * S mUn001 mU * S mGn001 mA * ST * SC * ST * SG * ST * RA * SG | GUUGATCTGTA | SnXSnX SSSSS RSSRO |
| 21329 | * ST * RAeoGeo m5Ceon001AeoGeon001 m5Ceo * STeo | GTAGCAGCT | OnXOnXS |
| WV- | mG * S mUn001 mU * S mGn001 mA * ST * ST * ST * SG * ST * RA * SG | GUUGATTTGTA | SnXSnX SSSSS |
| 21330 | * SC * RAeon001Geo m5Ceon001AeoGeon001 m5Ceo * STeo | GCAGCAGCT | RSSRnXOnXOnXS |
| WV- | mG * S mUn001 mU * S mGn001 mA * ST * SC * ST * SG * ST * RA * SG | GUUGATCTGTA | SnXSnX SSSSS |
| 21331 | * ST * RAeon001Geo m5Ceon001AeoGeon001 m5Ceo * STeo | GTAGCAGCT | RSSRnXOnXOnXS |
| WV- | mG * S mUn001 mU * S mGn001 mA * S mU * S mU * ST * SG * ST * RA | GUUGAUUTGTA | SnXSnX SSSSS RSSSRO |
| 21332 | * SG * SC * SA * RGeo m5Ceon001AeoGeon001 m5Ceo * STeo | GCAGCAGCT | nXOnXS |
| WV- | mG * S mUn001 mU * S mGn001 mA * S mU * S mC * ST * SG * ST * RA | GUUGAUCTGTA | SnXSnX SSSSS RSSSRO |
| 21333 | * SG * ST * SA * RGeo m5Ceon001AeoGeon001 m5Ceo * STeo | GTAGCAGCT | nXOnXS |
| WV- | mG * S mUn001 mU * S mGn001 mA * S mUn001 mU * ST * SG * ST * | GUUGAUUTGTA | SnXSnXSnX SSSR |
| 21334 | RA * SG * SC * SA * RGeo m5Ceon001AeoGeon001 m5Ceo * STeo | GCAGCAGCT | SSSRO nXOnXS |
| WV- | mG * S mUn001 mU * S mGn001 mA * S mUn001 mC * ST * SG * ST * | GUUGAUCTGTA | SnXSnXSnX SSSR |
| 21335 | RA * SG * ST * SA * RGeo m5Ceon001AeoGeon001 m5Ceo * STeo | GTAGCAGCT | SSSRO nXOnXS |
| WV- | mG * S mUn001 mU * S mGn001 mA * S mU * ST * ST * SG * ST * RA * | GUUGAUTTGTA | SnXSnX SSSSS RSSRO |
| 21336 | SG * SC * RAeoGeo m5Ceon001AeoGeon001 m5Ceo * STeo | GCAGCAGCT | OnXOnXS |
| WV- | mG * S mUn001 mU * S mGn001 mA * S mU * SC * ST * SG * ST * RA * | GUUGAUCTGTA | SnXSnX SSSSS RSSRO |
| 21337 | SG * ST * RAeoGeo m5Ceon001AeoGeon001 m5Ceo * STeo | GTAGCAGCT | OnXOnXS |
| WV- | mG * S mUn001 mU * S mGn001 mA * S mU * ST * ST * SG * ST * RA * | GUUGAUTTGTA | SnXSnX SSSSS |
| 21338 | SG * SC * RAeon001Geo m5Ceon001AeoGeon001 m5Ceo * STeo | GCAGCAGCT | RSSRnXOnXOnXS |
| WV- | mG * S mUn001 mU * S mGn001 mA * S mU * SC * ST * SG * ST * RA * | GUUGAUCTGTA | SnXSnX SSSSS |
| 21339 | SG * ST * RAeon001Geo m5Ceon001AeoGeon001 m5Ceo * STeo | GTAGCAGCT | RSSRnXOnXOnXS |
| WV- | mG * S mUn001 mU * S mGn001 mA * S mU * S mU * ST * SG * ST * RA | GUUGAUUTGTA | SnXSnX SSSSS RSSRO |
| 21340 | * SG * SC * RAeoGeo m5Ceon001AeoGeon001 m5Ceo * STeo | GCAGCAGCT | OnXOnXS |
| WV- | mG * S mUn001 mU * S mGn001 mA * S mU * S mC * ST * SG * ST * RA | GUUGAUCTGTA | SnXSnX SSSSS RSSRO |
| 21341 | * SG * ST * RAeoGeo m5Ceon001AeoGeon001 m5Ceo * STeo | GTAGCAGCT | OnXOnXS |
| WV- | mG * S mUn001 mU * S mGn001 mA * S mUn001 mU * ST * SG * ST * | GUUGAUUTGTA | SnXSnXSnX SSSRSSR |
| 21342 | RA * SG * SC * RAeon001Geo m5Ceon001AeoGeon001 m5Ceo * STeo | GCAGCAGCT | nXOnXOnXS |
| WV- | mG * S mUn001 mU * S mGn001 mA * S mUn001 mC * ST * SG * ST * | GUUGAUCTGTA | SnXSnXSnX SSSRSSR |
| 21343 | RA * SG * ST * RAeon001Geo m5Ceon001AeoGeon001 m5Ceo * STeo | GTAGCAGCT | nXOnXOnXS |
| WV- | mG * S mU * S mU * S mG * S mA * ST * SC * ST * SG * ST * RA * SG * | GUUGATCTGTA | SSSSS SSSSR SSSSR |
| 21391 | SC * SA * SG * R m5Ceon001SAeon001SGeon001S m5Ceo * STeo | GCAGCAGCT | nSnSnSS |
| WV- | mG * S mUn001R mU * S mGn001R mA * ST * SC * ST * SG * ST * RA * | GUUGATCTGTA | SnRSnR SSSSS RSSSSR |
| 21392 | SG * SC * SA * SG * R m5Ceon001SAeoGeon001S m5Ceo * STeo | GCAGCAGCT | nSOnSS |
| WV- | mG * S mUn001R mU mGn001R mA * ST * SC * ST * SG * ST * RA * SG | GUUGATCTGTA | SnROnR SSSSS RSSSSR |
| 21393 | * SC * SA * SG * R m5Ceon001SAeoGeon001S m5Ceo * STeo | GCAGCAGCT | nSOnSS |
| WV- | mG * S mUn001R mU mGn001R mA * ST * SC * ST * SG * ST * RA * SG | GUUGATCTGTA | SnROnR SSSSS RSSSSR |
| 21394 | * SC * SA * SG * R m5CeoAeoGeon001S m5Ceo * STeo | GCAGCAGCT | OOnSS |
| WV- | mG * S mU * S mU * S mG * S mA * ST * SC * ST * SG * ST * RA * SG * | GUUGATCTGTA | SSSSS SSSSR SSSSR |
| 21395 | ST * SA * SG * R m5Ceon001SAeon001SGeon001S m5Ceo * STeo | GTAGCAGCT | nSnSnSS |
| WV- | mG * S mU * S mUn001 mG * S mA * ST * SC * ST * SG * ST * RA * SG | GUUGATCTGTA | SSnX SSSSS SRSSSSR |
| 21396 | * SC * SA * SG * R m5CeoAeoGeon001S m5Ceo * STeo | GCAGCAGCT | OOnSS |
| WV- | mG * S mU * S mUn001 mG * S mA * ST * SC * ST * SG * ST * RA * SG | GUUGATCTGTA | SSnX SSSSS SRSSSSR |
| 21397 | * SC * SA * SG * R m5Ceon001SAeoGeo m5Ceo * STeo | GCAGCAGCT | nSOOS |
| WV- | mG * S mU * S mUn001R mG * S mA * ST * SC * ST * SG * ST * RA * | GUUGATCTGTA | SSnR SSSSS SRSSSSR |
| 21398 | SG * SC * SA * SG * R m5CeoAeoGeon001S m5Ceo * STeo | GCAGCAGCT | OOnSS |
| WV- | mG * S mU * S mUn001R mG * S mA * ST * SC * ST * SG * ST * RA * | GUUGATCTGTA | SSnR SSSSS SRSSSSR |
| 21399 | SG * SC * SA * SG * R m5Ceon001SAeoGeo m5Ceo * STeo | GCAGCAGCT | nSOOS |
| WV- | mG * S mUn001R mUn001R mGn001R mA * ST * SC * ST * SG * ST * RA | GUUGATCTGTA | SnRnRnR SSSSS |
| 21400 | * SG * SC * SA * SG * R m5Ceon001SAeon001SGeon001S m5Ceo * STeo | GCAGCAGCT | RSSSSR nSnSnSS |
| WV- | mG * S mUn001R mUn001R mGn001R mA * ST * SC * ST * SG * ST * RA | GUUGATCTGTA | SnRnRnR SSSSS |
| 21401 | * SG * ST * SA * SG * R m5Ceon001SAeon001SGeon001S m5Ceo * STeo | GTAGCAGCT | RSSSSR nSnSnSS |
| WV- | mG * S mUn001R mU * S mG mA * ST * SC * ST * SG * ST * RA * SG * | GUUGATCTGTA | SnRSO SSSSS RSSSSR |
| 21402 | SC * SA * SG * R m5Ceon001SAeoGeon001S m5Ceo * STeo | GCAGCAGCT | nSOnSS |
| WV- | mG * S mU * S mU * S mG * S mA * ST * SC * ST * SG * ST * RA * SG * | GUUGATCTGTA | SSSSS SSSSR SSSSR |
| 21403 | SC * SA * SG * R m5Ceon001RAeon001RGeon001R m5Ceo * STeo | GCAGCAGCT | nRnRnRS |
| WV- | mG * S mUn001R mU * S mGn001R mA * ST * SC * ST * SG * ST * RA * | GUUGATCTGTA | SnRSnR SSSSS RSSSSR |
| 21404 | SG * SC * SA * SG * R m5Ceon001RAeoGeon001R m5Ceo * STeo | GCAGCAGCT | nROnRS |
| WV- | mG * S mUn001R mU mGn001R mA * ST * SC * ST * SG * ST * RA * SG | GUUGATCTGTA | SnROnR SSSSS RSSSSR |
| 21405 | * SC * SA * SG * R m5Ceon001RAeoGeon001R m5Ceo * STeo | GCAGCAGCT | nROnRS |
| WV- | mG * S mUn001R mU mGn001R mA * ST * SC * ST * SG * ST * RA * SG | GUUGATCTGTA | SnROnR SSSSS RSSSSR |
| 21406 | * SC * SA * SG * R m5CeoAeoGeon001R m5Ceo * STeo | GCAGCAGCT | OOnRS |
| WV- | mG * S mU * S mU * S mG * S mA * ST * SC * ST * SG * ST * RA * SG * | GUUGATCTGTA | SSSSS SSSSR SSSSR |
| 21407 | ST * SA * SG * R m5Ceon001RAeon001RGeon001R m5Ceo * STeo | GTAGCAGCT | nRnRnRS |
| WV- | mG * S mU * S mUn001R mG * S mA * ST * SC * ST * SG * ST * RA * | GUUGATCTGTA | SSnR SSSSS SRSSSSR |
| 21408 | SG * SC * SA * SG * R m5CeoAeoGeon001R m5Ceo * STeo | GCAGCAGCT | OOnRS |
| WV- | mG * S mU * S mUn001R mG * S mA * ST * SC * ST * SG * ST * RA * | GUUGATCTGTA | SSnR SSSSS SRSSSSR |
| 21409 | SG * SC * SA * SG * R m5Ceon001RAeoGeo m5Ceo * STeo | GCAGCAGCT | nROOS |
| WV- | mG * S mUn001R mUn001R mGn001R mA * ST * SC * ST * SG * ST * RA | GUUGATCTGTA | SnRnRnR SSSSS |
| 21410 | * SG * SC * SA * SG * R m5Ceon001RAeon001RGeon001R m5Ceo * STeo | GCAGCAGCT | RSSSSR nRnRnRS |
| WV- | mG * S mUn001R mUn001R mGn001R mA * ST * SC * ST * SG * ST * RA | GUUGATCTGTA | SnRnRnR SSSSS |
| 21411 | * SG * ST * SA * SG * R m5Ceon001RAeon001RGeon001R m5Ceo * STeo | GTAGCAGCT | RSSSSR nRnRnRS |
| WV- | mG * S mUn001R mU * S mG mA * ST * SC * ST * SG * ST * RA * SG * | GUUGATCTGTA | SnRSO SSSSS RSSSSR |
| 21412 | SC * SA * SG * R m5Ceon001RAeoGeon001R m5Ceo * STeo | GCAGCAGCT | nROnRS |
| WV- | mG * SmU * SmU * SmG * SmA * ST * SC * ST * SG * ST * RA * SG * SC | GUUGATCTGTA | SSSSS SSSSR SSSSR |
| 12282 | * SA * SG * Rm5CeoAeoGeom5Ceo * STeo | GCAGCAGCT | OOOS |
| WV- | mG * SmU * SmU * SmG * SmA * ST * SC * ST * SG * ST * RA * SG * SC | GUUGATCTGTA | SSSSS SSSSR SSSSR |
| 14914 | * SA * SG * Rm5Ceon001Aeon001Geon001m5Ceo * STeo | GCAGCAGCT | nXnXnXS |
| WV- | mG * SmU * SmU * SmG * SmA * ST * SC * ST * SG * ST * SA * RG * SC | GUUGATCTGTA | SSSSS SSSSS |
| 12283 | * SA * SG * Rm5CeoAeoGeom5Ceo * STeo | GCAGCAGCT | RSSSROOOS |
| WV- | mG * SmU * SmU * SmG * SmA * ST * SC * ST * SG * RT * SA * SG * SC | GUUGATCTGTA | SSSSS SSSRSSSSS |
| 12281 | * SA * SG * Rm5CeoAeoGeom5Ceo * STeo | GCAGCAGCT | ROOOS |
| WV- | mG * SmU * SmU * SmG * SmA * ST * SC * ST * SG * ST * RA * SG * RC | GUUGATCTGTA | SSSSS SSSSR |
| 15080 | * SA * SG * Rm5Ceon001Aeon001Geon001m5Ceo * STeo | GCAGCAGCT | SRSSRnXnXnXS |
| WV- | mG * SmU * SmUn001mG * SmA * ST * SC * ST * SG * ST * RA * SG * | GUUGATCTGTA | SSnXSSSSS SRSSSSR |
| 17777 | SC * SA * SG * Rm5CeoAeoGeom5Ceo * STeo | GCAGCAGCT | OOOS |
| WV- | mG * SmU * SmU * SmG * SmA * ST * SC * ST * SG * ST * RA * SG * SC | GUUGATCTGTA | SSSSS SSSSR SSSSR |
| 17778 | * SA * SG * Rm5CeoAeon001Geom5Ceo * STeo | GCAGCAGCT | OnXOS |
| WV- | mG * SmU * SmUn001mG * SmA * ST * SC * ST * SG * ST * RA * SG * | GUUGATCTGTA | SSnXSSSSS SRSSSSR |
| 17779 | SC * SA * SG * Rm5CeoAeon001Geom5Ceo * STeo | GCAGCAGCT | OnXOS |
| WV- | mG * SmUn001mU * SmGn001mA * ST * SC * ST * SG * ST * RA * SG * | GUUGATCTGTA | SnXSnXSSSSS RSSSSR |
| 17780 | SC * SA * SG * Rm5CeoAeoGeom5Ceo * STeo | GCAGCAGCT | OOOS |
| WV- | mG * SmU * SmU * SmG * SmA * ST * SC * ST * SG * ST * RA * SG * SC | GUUGATCTGTA | SSSSS SSSSR SSSSR |
| 17781 | * SA * SG * Rm5Ceon001AeoGeon001m5Ceo * STeo | GCAGCAGCT | nXOnXS |
| WV- | mG * SmUn001mU * SmGn001mA * ST * SC * ST * SG * ST * RA * SG * | GUUGATCTGTA | SnXSnXSSSSS RSSSSR |
| 17782 | SC * SA * SG * Rm5Ceon001AeoGeon001m5Ceo * STeo | GCAGCAGCT | nXOnXS |
| WV- | Teo * RTeoGeoAeoTeo * RC * ST * SG * ST * SA * RG * SC * SA * SG * | TTGATCTGTA | ROOORSSSSR SSSSR |
| 14083 | SC * RAeo * SmG * SmC * SmU * SmU | GCAGCAGCUU | SSSS |
| WV- | Geo * RTeoTeoGeoAeo * RT * SC * ST * SG * ST * SA * RG * SC * SA * | GTTGATCTGTA | ROOORSSSSS |
| 14084 | SG * Rm5Ceo * SmA * SmG * SmC * SmU | GCAGCAGCU | RSSSRSSSS |
| WV- | Geo * RGeoTeoTeoGeo * RA * ST * SC * ST * SG * ST * SA * RG * SC * | GGTTGATCTGTA | ROOORSSSSS |
| 14085 | SA * RGeo * SmC * SmA * SmG * SmC | GCAGCAGC | SRSSRSSSS |
| WV- | mG * SmU * SmU * SmG * SAeo * RT * SC * ST * SG * ST * RA * SG * | GUUGATCTGTA | SSSSR SSSSR SSSSR |
| 14098 | SC * SA * SG * Rm5CeoAeoGeom5Ceo * STeo | GCAGCAGCT | OOOS |
| WV- | Geo * RTeoTeoGeoAeo * RT * SC * ST * SG * ST * RA * SG * SC * SA * | GTTGATCTGTA | ROOORSSSSR SSSSR |
| 14096 | SG * Rm5Ceo * SmA * SmG * SmC * SmU | GCAGCAGCU | SSSS |
| WV- | m51C * Aeo * m51C * Aeo * Aeo * G * G * G * C * G * C * A * G * A * C * | CACAAGGGCGC | XXXXX XXXXX |
| 13628 | Teo * Teo * m51C * m5Ceo * 1A | AGACTTCCA | XXXXX XXXX |
| WV- | mG * SmU * SmUmG * SmA * ST * SC * ST * SG * ST * RA * SG * SC * | GUUGATCTGT | SSOSS SSSSR SSSS |
| 21447 | SA * SG * Rm5Ceon001RAeon001RGeon001Rm5Ceo * STeo | AGCAGCAGCT | RnRnRnRS |
| WV- | mG * SmU * SmUmG * SmA * ST * SC * ST * SG * ST * RA * SG * ST * | GUUGATCTGT | SSOSS SSSSR SSSS |
| 21448 | SA * SG * Rm5Ceon001RAeon001RGeon001Rm5Ceo * STeo | AGTAGCAGCT | RnRnRnRS |
| WV- | mG * SmU * SmUmG * SmA * ST * SC * ST * SG * ST * RA * SG * SC * | GUUGATCTGT | SSOSS SSSSR SSSS |
| 21449 | SA * SG * Rm5Ceon001SAeon001SGeon001Sm5Ceo * STeo | AGCAGCAGCT | RnSnSnSS |
| WV- | mG * SmU * SmUmG * SmA * ST * SC * ST * SG * ST * RA * SG * ST * | GUUGATCTGT | SSOSS SSSSR SSSS |
| 21450 | SA * SG * Rm5Ceon001SAeon001SGeon001Sm5Ceo * STeo | AGTAGCAGCT | RnSnSnSS |
| WV- | mG * SmGmCmAmC * SA * RA * SG * SG * SG * SC * SA * SC * RA * | GGCACAAGGG | SOOOS RSSSS |
| 21465 | SG * SmAmCmUmU * SmC | CACAGACUUC | SSRSSOOOS |
| WV- | mG * SmGmCmAmC * SA * SA * RG * SG * SG * SC * SA * SC * RA * | GGCACAAGGG | SOOOS SRSSSS |
| 21466 | SG * SmAmCmUmU * SmC | CACAGACUUC | SRSSOOOS |
| WV- | mG * SmGmCmAmC * SA * SA * SG * RG * SG * SC * SA * SC * RA * | GGCACAAGGG | SOOOS SSRSSSS |
| 21467 | SG * SmAmCmUmU * SmC | CACAGACUUC | RSSOOOS |
| WV- | mG * SmGmCmAmC * SA * SA * SG * SG * RG * SC * SA * SC * RA * | GGCACAAGGG | SOOOS SSSRSSSR |
| 21468 | SG * SmAmCmUmU * SmC | CACAGACUUC | SSOOOS |
| WV- | mG * SmGmCmAmC * SA * SA * SG * SG * SG * RC * SA * SC * RA * | GGCACAAGGG | SOOOS SSSSR |
| 21469 | SG * SmAmCmUmU * SmC | CACAGACUUC | SSRSSOOOS |
| WV- | mG * SmGmCmAmC * SA * SA * SG * SG * SG * SC * SA * RC * RA * | GGCACAAGGG | SOOOS SSSSS SR |
| 21470 | SG * SmAmCmUmU * SmC | CACAGACUUC | RSSOOOS |
| WV- | mG * SmGmCmAmC * SA * SA * SG * SG * SG * SC * SA * SC * RA * | GGCACAAGGG | SOOOS SSSSS SSR |
| 21471 | RG * SmAmCmUmU * SmC | CACAGACUUC | RSOOOS |
| WV- | mG * SmGmCmAmC * SA * SA * SG * SG * SG * SC * SA * SC * RA * | GGCACAAGGG | SOOOS SSSSS SSR |
| 21472 | SG * RmAmCmUmU * SmC | CACAGACUUC | SROOOS |
| WV- | mG * SmUmGmCmA * SC * RA * SC * SA * SG * ST * SA * SG * RA * ST | GUGCACACAG | SOOOS RSSSS |
| 21474 | * SmGmAmGmG * SmG | TAGATGAGGG | SSRSSOOOS |
| WV- | mG * SmUmGmCmA * SC * SA * RC * SA * SG * ST * SA * SG * RA * ST | GUGCACACAG | SOOOS SRSSSS |
| 21475 | * SmGmAmGmG * SmG | TAGATGAGGG | SRSSOOOS |
| WV- | mG * SmUmGmCmA * SC * SA * SC * RA * SG * ST * SA * SG * RA * ST | GUGCACACAG | SOOOS SSRSSSS |
| 21476 | * SmGmAmGmG * SmG | TAGATGAGGG | RSSOOOS |
| WV- | mG * SmUmGmCmA * SC * SA * SC * SA * RG * ST * SA * SG * RA * ST | GUGCACACAG | SOOOS SSSRSSSR |
| 21477 | * SmGmAmGmG * SmG | TAGATGAGGG | SSOOOS |
| WV- | mG * SmUmGmCmA * SC * SA * SC * SA * SG * RT * SA * SG * RA * ST | GUGCACACAG | SOOOS SSSSR |
| 21478 | * SmGmAmGmG * SmG | TAGATGAGGG | SSRSSOOOS |
| WV- | mG * SmUmGmCmA * SC * SA * SC * SA * SG * ST * RA * SG * RA * ST | GUGCACACAG | SOOOS SSSSS R |
| 21479 | * SmGmAmGmG * SmG | TAGATGAGGG | SRSSOOOS |
| WV- | mG * SmUmGmCmA * SC * SA * SC * SA * SG * ST * SA * RG * RA * ST | GUGCACACAG | SOOOS SSSSS SR |
| 21480 | * SmGmAmGmG * SmG | TAGATGAGGG | RSSOOOS |
| WV- | mG * SmUmGmCmA * SC * SA * SC * SA * SG * ST * SA * SG * RA * RT | GUGCACACAG | SOOOS SSSSS SSR |
| 21481 | * SmGmAmGmG * SmG | TAGATGAGGG | RSOOOS |
| WV- | mG * SmUmGmCmA * SC * SA * SC * SA * SG * ST * SA * SG * RA * ST | GUGCACACAG | SOOOS SSSSS SSR |
| 21482 | * RmGmAmGmG * SmG | TAGATGAGGG | SROOOS |
| WV- | mGmGmCmAmC * SA * SA * SG * SG * SG * SC * SA * SC * RA * SG * | GGCACAAGGG | OOOOSSSSS SSSR |
| 21527 | SmAmCmUmU * SmC | CACAGACUUC | SSOOOS |
| WV- | mG * SmGmCmAmC * SA * SA * SA * SG * SG * SG * SC * SA * SC * | GGCACAAAGG | SOOOS SSSSS SSSR |
| 21528 | RA * SG * SmAmCmUmU * SmC | GCACAGACUUC | SSOOOS |
| WV- | mGmUmGmCmA * SC * SA * SC * SA * SG * ST * SA * SG * RA * ST * | GUGCACACAG | OOOOSSSSS SSSR |
| 21529 | SmGmAmGmG * SmG | TAGATGAGGG | SSOOOS |
| WV- | mG * SmUmGmCmA * SA * SC * SA * SC * SA * SG * ST * SA * SG * RA | GUGCAACACA | SOOOS SSSSS SSSR |
| 21530 | * ST * SmGmAmGmG * SmG | GTAGATGAGGG | SSOOOS |
| WV- | A * SA * SG * SG * SG * SC * SA * SC * RA * SG * SmAmCmUmU * | AAGGGCACAG | SSSSS SSR SSOOOS |
| 21531 | SmC | ACUUC | |
| WV- | A * SG * SG * SG * SC * SA * SC * RA * SG * SmAmCmUmU * SmC | AGGGCACAGA | SSSSS SR SSOOOS |
| 21532 | | CUUC | |
| WV- | G * SG * SG * SC * SA * SC * RA * SG * SmAmCmUmU * SmC | GGGCACAGAC UUC | SSSSS R SSOOOS |
| 21533 | | | |
| WV- | G * SG * SC * SA * SC * RA * SG * SmAmCmUmU * SmC | GGCACAGACU UC | SSSSR SSOOOS |
| 21534 | | | |
| WV- | G * SC * SA * SC * RA * SG * SmAmCmUmU * SmC | GCACAGACUU C | SSSRSSOOOS |
| 21535 | | | |
| WV- | C * SA * SC * RA * SG * SmAmCmUmU * SmC | CACAGACUUC | SSRSSOOOS |
| 21536 | | | |
| WV- | A * SC * RA * SG * SmAmCmUmU * SmC | ACAGACUUC | SRSSOOOS |
| 21537 | | | |
| WV- | C * RA * SG * SmAmCmUmU * SmC | CAGACUUC | RSSOOOS |
| 21538 | | | |
| WV- | A * SG * SmAmCmUmU * SmC | AGACUUC | SSOOOS |
| 21539 | | | |
| WV- | G * SmAmCmUmU * SmC | GACUUC | SOOOS |
| 21540 | | | |
| WV- | mc * RA * SA * SG * SG * SG * SC * SA * SC * RA * SG * | CAAGGGCACAG | RSSSS SSSRSSOOOS |
| 21541 | SmAmCmUmU * SmC | ACUUC | |
| WV- | A * RA * SG * SG * SG * SC * SA * SC * RA * SG * SmAmCmUmU * | AAGGGCACAG | RSSSS SSRSSOOOS |
| 21542 | SmC | ACUUC | |
| WV- | A * RG * SG * SG * SC * SA * SC * RA * SG * SmAmCmUmU * SmC | AGGGCACAGA | RSSSS SRSSOOOS |
| 21543 | | CUUC | |
| WV- | G * RG * SG * SC * SA * SC * RA * SG * SmAmCmUmU * SmC | GGGCACAGAC UUC | RSSSS RSSOOOS |
| 21544 | | | |
| WV-21545 | G * RG * SC * SA * SC * RA * SG * SmAmCmUmU * SmC | GGCACAGACU UC | RSSSR SSOOOS |
| WV-21546 | G * RC * SA * SC * RA * SG * SmAmCmUmU * SmC | GCACAGACUU C | RSSRS SOOOS |
| WV-21547 | C * RA * SC * RA * SG * SmAmCmUmU * SmC | CACAGACUUC | RSRSSOOOS |
| WV-21548 | A * RC * RA * SG * SmAmCmUmU * SmC | ACAGACUUC | RRSSOOOS |
| WV-21549 | C * SA * SG * SmAmCmUmU * SmC | CAGACUUC | SSSOOOS |
| WV-21550 | A * RG * SmAmCmUmU * SmC | AGACUUC | RSOOOS |
| WV-21551 | G * RmAmCmUmU * SmC | GACUUC | ROOOS |
| WV- | Teo * Sm5Ceon001Teon001m5Ceon001m5Ceo * RA * ST * ST * RC * ST * | TCTCCATTCT | SnXnXnXRS SRS SSS |
| 22213 | SA * ST * SC * RT * ST * SAeo * SmU * SmG * SmU * SmU | ATCTTAUGUU | RSSSS SS |
| WV- | m5Ceo * SAeom5CeoAeoGeo * RT * RA * SG * SA * ST * SG * | CACAGTAGATGAGGGAGCAG | SOOOR RSSSS |
| 22920 | SA * SG * SG * SG * SAeoGeom5CeoAeo * SGeo | | SSSSS OOOS |
| WV- | m5Ceo * SAeom5CeoAeoGeo * RT * SA * RG * SA * ST * SG * | CACAGTAGATGAGGGAGCAG | SOOOR SRSSS |
| 22921 | SA * SG * SG * SG * SAeoGeom5CeoAeo * SGeo | | SSSSS OOOS |
| WV- | m5Ceo * SAeom5CeoAeoGeo * RT * SA * SG * RA * ST * SG * | CACAGTAGATGAGGGAGCAG | SOOOR SSRSS |
| 22922 | SA * SG * SG * SG * SAeoGeom5CeoAeo * SGeo | | SSSSS OOOS |
| WV- | Aeo * Sm5CeoAeom5CeoAeo * RG * ST * RA * SG * SA * ST * | ACACAGTAGATGAGGGAGCA | SOOOR SRSSS |
| 22923 | SG * SA * SG * SG * SGeoAeoGeom5Ceo * SAeo | | SSSSS OOOS |
| WV- | Aeo * Sm5CeoAeom5CeoAeo * RG * ST * SA * RG * SA * ST * | ACACAGTAGATGAGGGAGCA | SOOOR SSRSS |
| 22924 | SG * SA * SG * SG * SGeoAeoGeom5Ceo * SAeo | | SSSSS OOOS |
| WV- | Aeo * Sm5CeoAeom5CeoAeo * RG * ST * SA * SG * RA * ST * | ACACAGTAGATGAGGGAGCA | SOOOR SSSRS |
| 22925 | SG * SA * SG * SG * SGeoAeoGeom5Ceo * SAeo | | SSSSS OOOS |
| WV- | m5Ceo * SAeom5CeoAeom5Ceo * RA * SG * ST * RA * SG * SA | CACACAGTAGATGAGGGAGC | SOOOR SSRSS |
| 22926 | * ST * SG * SA * SG * SGeoGeoAeoGeo * Sm5Ceo | | SSSSS OOOS |
| WV- | m5Ceo * SAeom5CeoAeom5Ceo * RA * SG * ST * SA * RG * SA | CACACAGTAGATGAGGGAGC | SOOOR SSSRS |
| 22927 | * ST * SG * SA * SG * SGeoGeoAeoGeo * Sm5Ceo | | SSSSS OOOS |
| WV- | m5Ceo * SAeom5CeoAeom5Ceo * RA * SG * ST * SA * SG * RA | CACACAGTAGATGAGGGAGC | SOOOR SSSSR |
| 22928 | * ST * SG * SA * SG * SGeoGeoAeoGeo * Sm5Ceo | | SSSSS OOOS |
| WV- | Geo * Sm5CeoAeom5CeoAeo * Rm5C * SA * SG * ST * RA * SG | GCACACAGTAGATGAGGGAG | SOOOR SSSRS |
| 22929 | * SA * ST * SG * SA * SGeoGeoGeoAeo * SGeo | | SSSSS OOOS |
| WV- | Geo * Sm5CeoAeom5CeoAeo * Rm5C * SA * SG * ST * SA * RG | GCACACAGTAGATGAGGGAG | SOOOR SSSSR |
| 22930 | * SA * ST * SG * SA * SGeoGeoGeoAeo * SGeo | | SSSSS OOOS |
| WV- | Geo * Sm5CeoAeom5CeoAeo * Rm5C * SA * SG * ST * SA * SG | GCACACAGTAGATGAGGGAG | SOOOR SSSSS |
| 22931 | * RA * ST * SG * SA * SGeoGeoGeoAeo * SGeo | | RSSSS OOOS |
| WV- | Teo * SGeom5CeoAeom5Ceo * RA * Sm5C * SA * SG * ST * RA | TGCACACAGTAGATGAGGGA | SOOOR SSSSR |
| 22932 | * SG * SA * ST * SG * SAeoGeoGeoGeo * SAeo | | SSSSS OOOS |
| WV- | Teo * SGeom5CeoAeom5Ceo * RA * Sm5C * SA * SG * ST * SA | TGCACACAGTAGATGAGGGA | SOOOR SSSSS |
| 22933 | * RG * SA * ST * SG * SAeoGeoGeoGeo * SAeo | | RSSSS OOOS |
| WV- | Teo * SGeom5CeoAeom5Ceo * RA * Sm5C * SA * SG * ST * SA | TGCACACAGTAGATGAGGGA | SOOOR SSSSS |
| 22934 | * SG * RA * ST * SG * SAeoGeoGeoGeo * SAeo | | SRSSS OOOS |
| WV- | Geo * STeoGeom5CeoAeo * Rm5C * SA * Sm5C * SA * SG * ST | GTGCACACAGTAGATGAGGG | SOOOR SSSSS |
| 22935 | * RA * SG * SA * ST * SGeoAeoGeoGeo * SGeo | | RSSSS OOOS |
| WV- | Geo * STeoGeom5CeoAeo * Rm5C * SA * Sm5C * SA * SG * ST | GTGCACACAGTAGATGAGGG | SOOOR SSSSS |
| 22936 | * SA * RG * SA * ST * SGeoAeoGeoGeo * SGeo | | SRSSS OOOS |
| WV- | Geo * STeoGeom5CeoAeo * Rm5C * SA * Sm5C * SA * SG * ST | GTGCACACAGTAGATGAGGG | SOOOR SSSSS |
| 22937 | * SA * SG * RA * ST * SGeoAeoGeoGeo * SGeo | | SSRSS OOOS |
| WV- | m5Ceo * SAeom5CeoAeoGeo * RT * RA * SG * SA * ST * SG * | CACAGTAGATGAGGGAGCAG | SOOOR RSSSS |
| 22938 | SA * SG * SG * SG * SmA * SmG * SmC * SmA * SmG | | SSSSS SSSS |
| WV- | m5Ceo * SAeom5CeoAeoGeo * RT * SA * RG * SA * ST * SG * | CACAGTAGATGAGGGAGCAG | SOOOR SRSSS |
| 22939 | SA * SG * SG * SG * SmA * SmG * SmC * SmA * SmG | | SSSSS SSSS |
| WV- | m5Ceo * SAeom5CeoAeoGeo * RT * SA * SG * RA * ST * SG * | CACAGTAGATGAGGGAGCAG | SOOOR SSRSS |
| 22940 | SA * SG * SG * SG * SmA * SmG * SmC * SmA * SmG | | SSSSS SSSS |
| WV- | Aeo * Sm5CeoAeom5CeoAeo * RG * ST * RA * SG * SA * ST * | ACACAGTAGATGAGGGAGCA | SOOOR SRSSS |
| 22941 | SG * SA * SG * SG * SmG * SmA * SmG * SmC * SmA | | SSSSS SSSS |
| WV- | Aeo * Sm5CeoAeom5CeoAeo * RG * ST * SA * RG * SA * ST * | ACACAGTAGATGAGGGAGCA | SOOOR SSRSS |
| 22942 | SG * SA * SG * SG * SmG * SmA * SmG * SmC * SmA | | SSSSS SSSS |
| WV- | Aeo * Sm5CeoAeom5CeoAeo * RG * ST * SA * SG * RA * ST * | ACACAGTAGATGAGGGAGCA | SOOOR SSSRS |
| 22943 | SG * SA * SG * SG * SmG * SmA * SmG * SmC * SmA | | SSSSS SSSS |
| WV- | m5Ceo * SAeom5CeoAeom5Ceo * RA * SG * ST * RA * SG * SA | CACACAGTAGATGAGGGAGC | SOOOR SSRSS |
| 22944 | * ST * SG * SA * SG * SmG * SmG * SmA * SmG * SmC | | SSSSS SSSS |
| WV- | m5Ceo * SAeom5CeoAeom5Ceo * RA * SG * ST * SA * RG * SA | CACACAGTAGATGAGGGAGC | SOOOR SSSRS |
| 22945 | * ST * SG * SA * SG * SmG * SmG * SmA * SmG * SmC | | SSSSS SSSS |
| WV- | m5Ceo * SAeom5CeoAeom5Ceo * RA * SG * ST * SA * SG * RA | CACACAGTAGATGAGGGAGC | SOOOR SSSSR |
| 22946 | * ST * SG * SA * SG * SmG * SmG * SmA * SmG * SmC | | SSSSS SSSS |
| WV- | Geo * Sm5CeoAeom5CeoAeo * Rm5C * SA * SG * ST * RA * SG | GCACACAGTAGATGAGGGAG | SOOOR SSSRS |
| 22947 | * SA * ST * SG * SA * SmG * SmG * SmG * SmA * SmG | | SSSSS SSSS |
| WV- | Geo * Sm5CeoAeom5CeoAeo * Rm5C * SA * SG * ST * SA * RG | GCACACAGTAGATGAGGGAG | SOOOR SSSSR |
| 22948 | * SA * ST * SG * SA * SmG * SmG * SmG * SmA * SmG | | SSSSS SSSS |
| WV- | Geo * Sm5CeoAeom5CeoAeo * Rm5C * SA * SG * ST * SA * SG | GCACACAGTAGATGAGGGAG | SOOOR SSSSS |
| 22949 | * RA * ST * SG * SA * SmG * SmG * SmG * SmA * SmG | | RSSSS SSSS |
| WV- | Teo * SGeom5CeoAeom5Ceo * RA * Sm5C * SA * SG * ST * RA | TGCACACAGTAGATGAGGGA | SOOOR SSSSR |
| 22950 | * SG * SA * ST * SG * SmA * SmG * SmG * SmG * SmA | | SSSSS SSSS |
| WV- | Teo * SGeom5CeoAeom5Ceo * RA * Sm5C * SA * SG * ST * SA | TGCACACAGTAGATGAGGGA | SOOOR SSSSS |
| 22951 | * RG * SA * ST * SG * SmA * SmG * SmG * SmG * SmA | | RSSSS SSSS |
| WV- | Teo * SGeom5CeoAeom5Ceo * RA * Sm5C * SA * SG * ST * SA | TGCACACAGTAGATGAGGGA | SOOOR SSSSS |
| 22952 | * SG * RA * ST * SG * SmA * SmG * SmG * SmG * SmA | | SRSSS SSSS |
| WV- | Geo * STeoGeom5CeoAeo * Rm5C * SA * Sm5C * SA * SG * ST | GTGCACACAGTAGATGAGGG | SOOOR SSSSS |
| 22953 | * RA * SG * SA * ST * SmG * SmA * SmG * SmG * SmG | | RSSSS SSSS |
| WV- | Geo * STeoGeom5CeoAeo * Rm5C * SA * Sm5C * SA * SG * ST | GTGCACACAGTAGATGAGGG | SOOOR SSSSS |
| 22954 | * SA * RG * SA * ST * SmG * SmA * SmG * SmG * SmG | | SRSSS SSSS |
| WV- | Geo * STeoGeom5CeoAeo * Rm5C * SA * Sm5C * SA * SG * ST | GTGCACACAGTAGATGAGGG | SOOOR SSSSS |
| 22955 | * SA * SG * RA * ST * SmG * SmA * SmG * SmG * SmG | | SSRSS SSSS |
| WV- | mC * SmA * SmC * SmA * SmG * ST * RA * SG * SA * ST * SG | CACAGTAGATGAGGGAGCAG | SSSSS RSSSS |
| 22956 | * SA * SG * SG * SG * SAeoGeom5CeoAeo * SGeo | | SSSSS OOOS |
| WV- | mC * SmA * SmC * SmA * SmG * ST * SA * RG * SA * ST * SG | CACAGTAGATGAGGGAGCAG | SSSSS SRSSS |
| 22957 | * SA * SG * SG * SG * SAeoGeom5CeoAeo * SGeo | | SSSSS OOOS |
| WV- | mC * SmA * SmC * SmA * SmG * ST * SA * SG * RA * ST * SG | CACAGTAGATGAGGGAGCAG | SSSSS SSRSS |
| 22958 | * SA * SG * SG * SG * SAeoGeom5CeoAeo * SGeo | | SSSSS OOOS |
| WV- | mA * SmC * SmA * SmC * SmA * SG * ST * RA * SG * SA * ST | ACACAGTAGATGAGGGAGCA | SSSSS SRSSS |
| 22959 | * SG * SA * SG * SG * SGeoAeoGeom5Ceo * SAeo | | SSSSS OOOS |
| WV- | mA * SmC * SmA * SmC * SmA * SG * ST * SA * RG * SA * ST | ACACAGTAGATGAGGGAGCA | SSSSS SSRSS |
| 22960 | * SG * SA * SG * SG * SGeoAeoGeom5Ceo * SAeo | | SSSSS OOOS |
| WV- | mA * SmC * SmA * SmC * SmA * SG * ST * SA * SG * RA * ST | ACACAGTAGATGAGGGAGCA | SSSSS SSSRS |
| 22961 | * SG * SA * SG * SG * SGeoAeoGeom5Ceo * SAeo | | SSSSS OOOS |
| WV- | mC * SmA * SmC * SmA * SmC * SA * SG * ST * RA * SG * SA | CACACAGTAGATGAGGGAGC | SSSSS SSRSS |
| 22962 | * ST * SG * SA * SG * SGeoGeoAeoGeo * Sm5Ceo | | SSSSS OOOS |
| WV- | mC * SmA * SmC * SmA * SmC * SA * SG * ST * SA * RG * SA | CACACAGTAGATGAGGGAGC | SSSSS SSSRS |
| 22963 | * ST * SG * SA * SG * SGeoGeoAeoGeo * Sm5Ceo | | SSSSS OOOS |
| WV- | mC * SmA * SmC * SmA * SmC * SA * SG * ST * SA * SG * RA | CACACAGTAGATGAGGGAGC | SSSSS SSSSR |
| 22964 | * ST * SG * SA * SG * SGeoGeoAeoGeo * Sm5Ceo | | SSSSS OOOS |
| WV- | mG * SmC * SmA * SmC * SmA * Sm5C * SA * SG * ST * RA * | GCACACAGTAGATGAGGGAG | SSSSS SSSRS |
| 22965 | SG * SA * ST * SG * SA * SGeoGeoGeoAeo * SGeo | | SSSSS OOOS |
| WV- | mG * SmC * SmA * SmC * SmA * Sm5C * SA * SG * ST * SA * | GCACACAGTAGATGAGGGAG | SSSSS SSSSR |
| 22966 | RG * SA * ST * SG * SA * SGeoGeoGeoAeo * SGeo | | SSSSS OOOS |
| WV- | mG * SmC * SmA * SmC * SmA * Sm5C * SA * SG * ST * SA * | GCACACAGTAGATGAGGGAG | SSSSS SSSSS |
| 22967 | SG * RA * ST * SG * SA * SGeoGeoGeoAeo * SGeo | | RSSSS OOOS |
| WV- | mU * SmG * SmC * SmA * SmC * SA * Sm5C * SA * SG * ST * | UGCACACAGTAGATGAGGGA | SSSSS SSSSR |
| 22968 | RA * SG * SA * ST * SG * SAeoGeoGeoGeo * SAeo | | SSSSS OOOS |
| WV- | mU * SmG * SmC * SmA * SmC * SA * Sm5C * SA * SG * ST * | UGCACACAGTAGATGAGGGA | SSSSS SSSSS |
| 22969 | SA * RG * SA * ST * SG * SAeoGeoGeoGeo * SAeo | | RSSSS OOOS |
| WV- | mU * SmG * SmC * SmA * SmC * SA * Sm5C * SA * SG * ST * | UGCACACAGTAGATGAGGGA | SSSSS SSSSS |
| 22970 | SA * SG * RA * ST * SG * SAeoGeoGeoGeo * SAeo | | SRSSS OOOS |
| WV- | mG * SmU * SmG * SmC * SmA * Sm5C * SA * Sm5C * SA * SG | GUGCACACAGTAGATGAGGG | SSSSS SSSSS |
| 22971 | * ST * RA * SG * SA * ST * SGeoAeoGeoGeo * SGeo | | RSSSS OOOS |
| WV- | mG * SmU * SmG * SmC * SmA * Sm5C * SA * Sm5C * SA * SG | GUGCACACAGTAGATGAGGG | SSSSS SSSSS |
| 22972 | * ST * SA * RG * SA * ST * SGeoAeoGeoGeo * SGeo | | SRSSS OOOS |
| WV- | mG * SmU * SmG * SmC * SmA * Sm5C * SA * Sm5C * SA * SG | GUGCACACAGTAGATGAGGG | SSSSS SSSSS |
| 22973 | * ST * SA * SG * RA * ST * SGeoAeoGeoGeo * SGeo | | SSRSS OOOS |
| WV- | m5Ceo * SAeon001m5Ceon001Aeon001Geo * RT * RA * SG * SA | CACAGTAGATGAGGGAGCAG | SnXnXnXR RSSSS |
| 22974 | * ST * SG * SA * SG * SG * SG * SAeoGeom5CeoAeo * SGeo | | SSSSS OOOS |
| WV- | m5Ceo * SAeon001m5Ceon001Aeon001Geo * RT * SA * RG * SA | CACAGTAGATGAGGGAGCAG | SnXnXnXR SRSSS |
| 22975 | * ST * SG * SA * SG * SG * SG * SAeoGeom5CeoAeo * SGeo | | SSSSS OOOS |
| WV- | m5Ceo * SAeon001m5Ceon001Aeon001Geo * RT * SA * SG * RA | CACAGTAGATGAGGGAGCAG | SnXnXnXR SSRSS |
| 22976 | * ST * SG * SA * SG * SG * SG * SAeoGeom5CeoAeo * SGeo | | SSSSS OOOS |
| WV- | Aeo * Sm5Ceon001Aeon001m5Ceon001Aeo * RG * ST * RA * SG | ACACAGTAGATGAGGGAGCA | SnXnXnXR SRSSS |
| 22977 | * SA * ST * SG * SA * SG * SG * SGeoAeoGeom5Ceo * SAeo | | SSSSS OOOS |
| WV- | Aeo * Sm5Ceon001Aeon001m5Ceon001Aeo * RG * ST * SA * RG | ACACAGTAGATGAGGGAGCA | SnXnXnXR SSRSS |
| 22978 | * SA * ST * SG * SA * SG * SG * SGeoAeoGeom5Ceo * SAeo | | SSSSS OOOS |
| WV- | Aeo * Sm5Ceon001Aeon001m5Ceon001Aeo * RG * ST * SA * SG | ACACAGTAGATGAGGGAGCA | SnXnXnXR SSSRS |
| 22979 | * RA * ST * SG * SA * SG * SG * SGeoAeoGeom5Ceo * SAeo | | SSSSS OOOS |
| WV- | m5Ceo * SAeon001m5Ceon001Aeon001m5Ceo * RA * SG * ST * | CACACAGTAGATGAGGGAGC | SnXnXnXR SSRSS |
| 22980 | RA * SG * SA * ST * SG * SA * SG * SGeoGeoAeoGeo * Sm5Ceo | | SSSSS OOOS |
| WV- | m5Ceo * SAeon001m5Ceon001Aeon001m5Ceo * RA * SG * ST * | CACACAGTAGATGAGGGAGC | SnXnXnXR SSSRS |
| 22981 | SA * RG * SA * ST * SG * SA * SG * SGeoGeoAeoGeo * Sm5Ceo | | SSSSS OOOS |
| WV- | m5Ceo * SAeon001m5Ceon001Aeon001m5Ceo * RA * SG * ST * | CACACAGTAGATGAGGGAGC | SnXnXnXR SSSSR |
| 22982 | SA * SG * RA * ST * SG * SA * SG * SGeoGeoAeoGeo * Sm5Ceo | | SSSSS OOOS |
| WV- | Geo * Sm5Ceon001Aeon001m5Ceon001Aeo * Rm5C * SA * SG * | GCACACAGTAGATGAGGGAG | SnXnXnXR SSSRS |
| 22983 | ST * RA * SG * SA * ST * SG * SA * SGeoGeoGeoAeo * SGeo | | SSSSS OOOS |
| WV- | Geo * Sm5Ceon001Aeon001m5Ceon001Aeo * Rm5C * SA * SG * | GCACACAGTAGATGAGGGAG | SnXnXnXR SSSSR |
| 22984 | ST * SA * RG * SA * ST * SG * SA * SGeoGeoGeoAeo * SGeo | | SSSSS OOOS |
| WV- | Geo * Sm5Ceon001Aeon001m5Ceon001Aeo * Rm5C * SA * SG * | GCACACAGTAGATGAGGGAG | SnXnXnXR SSSSS |
| 22985 | ST * SA * SG * RA * ST * SG * SA * SGeoGeoGeoAeo * SGeo | | RSSSS OOOS |
| WV- | Teo * SGeon001m5Ceon001Aeon001m5Ceo * RA * Sm5C * SA * | TGCACACAGTAGATGAGGGA | SnXnXnXR SSSSR |
| 22986 | SG * ST * RA * SG * SA * ST * SG * SAeoGeoGeoGeo * SAeo | | SSSSS OOOS |
| WV- | Teo * SGeon001m5Ceon001Aeon001m5Ceo * RA * Sm5C * SA * | TGCACACAGTAGATGAGGGA | SnXnXnXR SSSSS |
| 22987 | SG * ST * SA * RG * SA * ST * SG * SAeoGeoGeoGeo * SAeo | | RSSSS OOOS |
| WV- | Teo * SGeon001m5Ceon001Aeon001m5Ceo * RA * Sm5C * SA * | TGCACACAGTAGATGAGGGA | SnXnXnXR SSSSS |
| 22988 | SG * ST * SA * SG * RA * ST * SG * SAeoGeoGeoGeo * SAeo | | SRSSS OOOS |
| WV- | Geo * STeon001Geon001m5Ceon001Aeo * Rm5C * SA * Sm5C * | GTGCACACAGTAGATGAGGG | SnXnXnXR SSSSS |
| 22989 | SA * SG * ST * RA * SG * SA * ST * SGeoAeoGeoGeo * SGeo | | RSSSS OOOS |
| WV- | Geo * STeon001Geon001m5Ceon001Aeo * Rm5C * SA * Sm5C * | GTGCACACAGTAGATGAGGG | SnXnXnXR SSSSS |
| 22990 | SA * SG * ST * SA * RG * SA * ST * SGeoAeoGeoGeo * SGeo | | SRSSS OOOS |
| WV- | Geo * STeon001Geon001m5Ceon001Aeo * Rm5C * SA * Sm5C * | GTGCACACAGTAGATGAGGG | SnXnXnXR SSSSS |
| 22991 | SA * SG * ST * SA * SG * RA * ST * SGeoAeoGeoGeo * SGeo | | SSRSS OOOS |
| WV- | m5Ceo * SAeom5CeoAeoGeo * RT * RA * SG * SA * ST * SG * | CACAGTAGATGAGGGAGCAG | SOOOR RSSSS |
| 22992 | SA * SG * SG * SG * SAeon001Geon001m5Ceon001Aeo * SGeo | | SSSSS nXnXnXS |
| WV- | m5Ceo * SAeom5CeoAeoGeo * RT * SA * RG * SA * ST * SG * | CACAGTAGATGAGGGAGCAG | SOOOR SRSSS |
| 22993 | SA * SG * SG * SG * SAeon001Geon001m5Ceon001Aeo * SGeo | | SSSSS nXnXnXS |
| WV- | m5Ceo * SAeom5CeoAeoGeo * RT * SA * SG * RA * ST * SG * | CACAGTAGATGAGGGAGCAG | SOOOR SSRSS |
| 22994 | SA * SG * SG * SG * SAeon001Geon001m5Ceon001Aeo * SGeo | | SSSSS nXnXnXS |
| WV- | Aeo * Sm5CeoAeom5CeoAeo * RG * ST * RA * SG * SA * ST * | ACACAGTAGATGAGGGAGCA | SOOOR SRSSS |
| 22995 | SG * SA * SG * SG * SGeon001Aeon001Geon001m5Ceo * SAeo | | SSSSS nXnXnXS |
| WV- | Aeo * Sm5CeoAeom5CeoAeo * RG * ST * SA * RG * SA * ST * | ACACAGTAGATGAGGGAGCA | SOOOR SSRSS |
| 22996 | SG * SA * SG * SG * SGeon001Aeon001Geon001m5Ceo * SAeo | | SSSSS nXnXnXS |
| WV- | Aeo * Sm5CeoAeom5CeoAeo * RG * ST * SA * SG * RA * ST * | ACACAGTAGATGAGGGAGCA | SOOOR SSSRS |
| 22997 | SG * SA * SG * SG * SGeon001Aeon001Geon001m5Ceo * SAeo | | SSSSS nXnXnXS |
| WV- | m5Ceo * SAeom5CeoAeom5Ceo * RA * SG * ST * RA * SG * SA | CACACAGTAGATGAGGGAGC | SOOOR SSRSS |
| 22998 | * ST * SG * SA * SG * SGeon001Geon001Aeon001Geo * Sm5Ceo | | SSSSS nXnXnXS |
| WV- | m5Ceo * SAeom5CeoAeom5Ceo * RA * SG * ST * SA * RG * SA | CACACAGTAGATGAGGGAGC | SOOOR SSSRS |
| 22999 | * ST * SG * SA * SG * SGeon001Geon001Aeon001Geo * Sm5Ceo | | SSSSS nXnXnXS |
| WV- | m5Ceo * SAeom5CeoAeom5Ceo * RA * SG * ST * SA * SG * RA | CACACAGTAGATGAGGGAGC | SOOOR SSSSR |
| 23000 | * ST * SG * SA * SG * SGeon001Geon001Aeon001Geo * Sm5Ceo | | SSSSS nXnXnXS |
| WV- | Geo * Sm5CeoAeom5CeoAeo * Rm5C * SA * SG * ST * RA * SG | GCACACAGTAGATGAGGGAG | SOOOR SSSRS |
| 23001 | * SA * ST * SG * SA * SGeon001Geon001Geon001Aeo * SGeo | | SSSSS nXnXnXS |
| WV- | Geo * Sm5CeoAeom5CeoAeo * Rm5C * SA * SG * ST * SA * RG | GCACACAGTAGATGAGGGAG | SOOOR SSSSR |
| 23002 | * SA * ST * SG * SA * SGeon001Geon001Geon001Aeo * SGeo | | SSSSS nXnXnXS |
| WV- | Geo * Sm5CeoAeom5CeoAeo * Rm5C * SA * SG * ST * SA * SG | GCACACAGTAGATGAGGGAG | SOOOR SSSSS |
| 23003 | * RA * ST * SG * SA * SGeon001Geon001Geon001Aeo * SGeo | | RSSSS nXnXnXS |
| WV- | Teo * SGeom5CeoAeom5Ceo * RA * Sm5C * SA * SG * ST * RA | TGCACACAGTAGATGAGGGA | SOOOR SSSSR |
| 23004 | * SG * SA * ST * SG * SAeon001Geon001Geon001Geo * SAeo | | SSSSS nXnXnXS |
| WV- | Teo * SGeom5CeoAeom5Ceo * RA * Sm5C * SA * SG * ST * SA | TGCACACAGTAGATGAGGGA | SOOOR SSSSS |
| 23005 | * RG * SA * ST * SG * SAeon001Geon001Geon001Geo * SAeo | | RSSSS nXnXnXS |
| WV- | Teo * SGeom5CeoAeom5Ceo * RA * Sm5C * SA * SG * ST * SA | TGCACACAGTAGATGAGGGA | SOOOR SSSSS |
| 23006 | * SG * RA * ST * SG * SAeon001Geon001Geon001Geo * SAeo | | SRSSS nXnXnXS |
| WV- | Geo * STeoGeom5CeoAeo * Rm5C * SA * Sm5C * SA * SG * ST | GTGCACACAGTAGATGAGGG | SOOOR SSSSS |
| 23007 | * RA * SG * SA * ST * SGeon001Aeon001Geon001Geo * SGeo | | RSSSS nXnXnXS |
| WV- | Geo * STeoGeom5CeoAeo * Rm5C * SA * Sm5C * SA * SG * ST | GTGCACACAGTAGATGAGGG | SOOOR SSSSS |
| 23008 | * SA * RG * SA * ST * SGeon001Aeon001Geon001Geo * SGeo | | SRSSS nXnXnXS |
| WV- | Geo * STeoGeom5CeoAeo * Rm5C * SA * Sm5C * SA * SG * ST | GTGCACACAGTAGATGAGGG | SOOOR SSSSS |
| 23009 | * SA * SG * RA * ST * SGeon001Aeon001Geon001Geo * SGeo | | SSRSS nXnXnXS |
| WV- | m5Ceo * SAeon001m5Ceon001Aeon001Geo * RT * RA * SG * SA * | CACAGTAGATGAGGGAGCAG | SnXnXnXR RSSSS |
| 23010 | ST * SG * SA * SG * SG * SG * SmA * SmG * SmC * SmA * SmG | | SSSSS SSSS |
| WV- | m5Ceo * SAeon001m5Ceon001Aeon001Geo * RT * SA * RG * SA * | CACAGTAGATGAGGGAGCAG | SnXnXnXR SRSSS |
| 23011 | ST * SG * SA * SG * SG * SG * SmA * SmG * SmC * SmA * SmG | | SSSSS SSSS |
| WV- | m5Ceo * SAeon001m5Ceon001Aeon001Geo * RT * SA * SG * RA * | CACAGTAGATGAGGGAGCAG | SnXnXnXR SSRSS |
| 23012 | ST * SG * SA * SG * SG * SG * SmA * SmG * SmC * SmA * SmG | | SSSSS SSSS |
| WV- | Aeo * Sm5Ceon001Aeon001m5Ceon001Aeo * RG * ST * RA * SG * | ACACAGTAGATGAGGGAGCA | SnXnXnXR SRSSS |
| 23013 | SA * ST * SG * SA * SG * SG * SmG * SmA * SmG * SmC * SmA | | SSSSS SSSS |
| WV- | Aeo * Sm5Ceon001Aeon001m5Ceon001Aeo * RG * ST * SA * RG * | ACACAGTAGATGAGGGAGCA | SnXnXnXR SSRSS |
| 23014 | SA * ST * SG * SA * SG * SG * SmG * SmA * SmG * SmC * SmA | | SSSSS SSSS |
| WV- | Aeo * Sm5Ceon001Aeon001m5Ceon001Aeo * RG * ST * SA * SG * | ACACAGTAGATGAGGGAGCA | SnXnXnXR SSSRS |
| 23015 | RA * ST * SG * SA * SG * SG * SmG * SmA * SmG * SmC * SmA | | SSSSS SSSS |
| WV- | m5Ceo * SAeon001m5Ceon001Aeon001m5Ceo * RA * SG * ST * RA | CACACAGTAGATGAGGGAGC | SnXnXnXR SSRSS |
| 23016 | * SG * SA * ST * SG * SA * SG * SmG * SmG * SmA * SmG * SmC | | SSSSS SSSS |
| WV- | m5Ceo * SAeon001m5Ceon001Aeon001m5Ceo * RA * SG * ST * SA | CACACAGTAGATGAGGGAGC | SnXnXnXR SSSRS |
| 23017 | * RG * SA * ST * SG * SA * SG * SmG * SmG * SmA * SmG * SmC | | SSSSS SSSS |
| WV- | m5Ceo * SAeon001m5Ceon001Aeon001m5Ceo * RA * SG * ST * SA | CACACAGTAGATGAGGGAGC | SnXnXnXR SSSSR |
| 23018 | * SG * RA * ST * SG * SA * SG * SmG * SmG * SmA * SmG * SmC | | SSSSS SSSS |
| WV- | Geo * Sm5Ceon001Aeon001m5Ceon001Aeo * Rm5C * SA * SG * ST | GCACACAGTAGATGAGGGAG | SnXnXnXR SSSRS |
| 23019 | * RA * SG * SA * ST * SG * SA * SmG * SmG * SmG * SmA * SmG | | SSSSS SSSS |
| WV- | Geo * Sm5Ceon001Aeon001m5Ceon001Aeo * Rm5C * SA * SG * ST | GCACACAGTAGATGAGGGAG | SnXnXnXR SSSSR |
| 23020 | * SA * RG * SA * ST * SG * SA * SmG * SmG * SmG * SmA * SmG | | SSSSS SSSS |
| WV- | Geo * Sm5Ceon001Aeon001m5Ceon001Aeo * Rm5C * SA * SG * ST | GCACACAGTAGATGAGGGAG | SnXnXnXR SSSSS |
| 23021 | * SA * SG * RA * ST * SG * SA * SmG * SmG * SmG * SmA * SmG | | RSSSS SSSS |
| WV- | Teo * SGeon001m5Ceon001Aeon001m5Ceo * RA * Sm5C * SA * SG | TGCACACAGTAGATGAGGGA | SnXnXnXR SSSSR |
| 23022 | * ST * RA * SG * SA * ST * SG * SmA * SmG * SmG * SmG * SmA | | SSSSS SSSS |
| WV- | Teo * SGeon001m5Ceon001Aeon001m5Ceo * RA * Sm5C * SA * SG | TGCACACAGTAGATGAGGGA | SnXnXnXR SSSSS |
| 23023 | * ST * SA * RG * SA * ST * SG * SmA * SmG * SmG * SmG * SmA | | RSSSS SSSS |
| WV- | Teo * SGeon001m5Ceon001Aeon001m5Ceo * RA * Sm5C * SA * SG | TGCACACAGTAGATGAGGGA | SnXnXnXR SSSSS |
| 23024 | * ST * SA * SG * RA * ST * SG * SmA * SmG * SmG * SmG * SmA | | SRSSS SSSS |
| WV- | Geo * STeon001Geon001m5Ceon001Aeo * Rm5C * SA * Sm5C * SA | GTGCACACAGTAGATGAGGG | SnXnXnXR SSSSS |
| 23025 | * SG * ST * RA * SG * SA * ST * SmG * SmA * SmG * SmG * SmG | | RSSSS SSSS |
| WV- | Geo * STeon001Geon001m5Ceon001Aeo * Rm5C * SA * Sm5C * SA | GTGCACACAGTAGATGAGGG | SnXnXnXR SSSSS |
| 23026 | * SG * ST * SA * RG * SA * ST * SmG * SmA * SmG * SmG * SmG | | SRSSS SSSS |
| WV- | Geo * STeon001Geon001m5Ceon001Aeo * Rm5C * SA * Sm5C * SA | GTGCACACAGTAGATGAGGG | SnXnXnXR SSSSS |
| 23027 | * SG * ST * SA * SG * RA * ST * SmG * SmA * SmG * SmG * SmG | | SSRSS SSSS |
| WV- | mC * SmA * SmC * SmA * SmG * ST * RA * SG * SA * ST * SG | CACAGTAGATGAGGGAGCAG | SSSSS RSSSS |
| 23028 | * SA * SG * SG * SG * SAeon001Geon001m5Ceon001Aeo * SGeo | | SSSSS nXnXnXS |
| WV- | mC * SmA * SmC * SmA * SmG * ST * SA * RG * SA * ST * SG | CACAGTAGATGAGGGAGCAG | SSSSS SRSSS |
| 23029 | * SA * SG * SG * SG * SAeon001Geon001m5Ceon001Aeo * SGeo | | SSSSS nXnXnXS |
| WV- | mC * SmA * SmC * SmA * SmG * ST * SA * SG * RA * ST * SG | CACAGTAGATGAGGGAGCAG | SSSSS SSRSS |
| 23030 | * SA * SG * SG * SG * SAeon001Geon001m5Ceon001Aeo * SGeo | | SSSSS nXnXnXS |
| WV- | mA * SmC * SmA * SmC * SmA * SG * ST * RA * SG * SA * ST | ACACAGTAGATGAGGGAGCA | SSSSS SRSSS |
| 23031 | * SG * SA * SG * SG * SGeon001Aeon001Geon001m5Ceo * SAeo | | SSSSS nXnXnXS |
| WV- | mA * SmC * SmA * SmC * SmA * SG * ST * SA * RG * SA * ST | ACACAGTAGATGAGGGAGCA | SSSSS SSRSS |
| 23032 | * SG * SA * SG * SG * SGeon001Aeon001Geon001m5Ceo * SAeo | | SSSSS nXnXnXS |
| WV- | mA * SmC * SmA * SmC * SmA * SG * ST * SA * SG * RA * ST | ACACAGTAGATGAGGGAGCA | SSSSS SSSRS |
| 23033 | * SG * SA * SG * SG * SGeon001Aeon001Geon001m5Ceo * SAeo | | SSSSS nXnXnXS |
| WV- | mC * SmA * SmC * SmA * SmC * SA * SG * ST * RA * SG * SA | CACACAGTAGATGAGGGAGC | SSSSS SSRSS |
| 23034 | * ST * SG * SA * SG * SGeon001Geon001Aeon001Geo * Sm5Ceo | | SSSSS nXnXnXS |
| WV- | mC * SmA * SmC * SmA * SmC * SA * SG * ST * SA * RG * SA | CACACAGTAGATGAGGGAGC | SSSSS SSSRS |
| 23035 | * ST * SG * SA * SG * SGeon001Geon001Aeon001Geo * Sm5Ceo | | SSSSS nXnXnXS |
| WV- | mC * SmA * SmC * SmA * SmC * SA * SG * ST * SA * SG * RA | CACACAGTAGATGAGGGAGC | SSSSS SSSSR |
| 23036 | * ST * SG * SA * SG * SGeon001Geon001Aeon001Geo * Sm5Ceo | | SSSSS nXnXnXS |
| WV- | mG * SmC * SmA * SmC * SmA * Sm5C * SA * SG * ST * RA * | GCACACAGTAGATGAGGGAG | SSSSS SSSRS |
| 23037 | SG * SA * ST * SG * SA * SGeon001Geon001Geon001Aeo * | | SSSSS nXnXnXS |
| | SGeo | | |
| WV- | mG * SmC * SmA * SmC * SmA * Sm5C * SA * SG * ST * SA * | GCACACAGTAGATGAGGGAG | SSSSS SSSSR |
| 23038 | RG * SA * ST * SG * SA * SGeon001Geon001Geon001Aeo * | | SSSSS nXnXnXS |
| | SGeo | | |
| WV- | mG * SmC * SmA * SmC * SmA * Sm5C * SA * SG * ST * SA * | GCACACAGTAGATGAGGGAG | SSSSS SSSSS |
| 23039 | SG * RA * ST * SG * SA * SGeon001Geon001Geon001Aeo * | | RSSSS nXnXnXS |
| | SGeo | | |
| WV- | mU * SmG * SmC * SmA * SmC * SA * Sm5C * SA * SG * ST * | UGCACACAGTAGATGAGGGA | SSSSS SSSSR |
| 23040 | RA * SG * SA * ST * SG * SAeon001Geon001Geon001Geo * | | SSSSS nXnXnXS |
| | SAeo | | |
| WV- | mU * SmG * SmC * SmA * SmC * SA * Sm5C * SA * SG * ST * | UGCACACAGTAGATGAGGGA | SSSSS SSSSS |
| 23041 | SA * RG * SA * ST * SG * SAeon001Geon001Geon001Geo * | | RSSSS nXnXnXS |
| | SAeo | | |
| WV- | mU * SmG * SmC * SmA * SmC * SA * Sm5C * SA * SG * ST * | UGCACACAGTAGATGAGGGA | SSSSS SSSSS |
| 23042 | SA * SG * RA * ST * SG * SAeon001Geon001Geon001Geo * | | SRSSS nXnXnXS |
| | SAeo | | |
| WV- | mG * SmU * SmG * SmC * SmA * Sm5C * SA * Sm5C * SA * SG | GUGCACACAGTAGATGAGGG | SSSSS SSSSS |
| 23043 | * ST * RA * SG * SA * ST * SGeon001Aeon001Geon001Geo * | | RSSSS nXnXnXS |
| | SGeo | | |
| WV- | mG * SmU * SmG * SmC * SmA * Sm5C * SA * Sm5C * SA * SG | GUGCACACAGTAGATGAGGG | SSSSS SSSSS |
| 23044 | * ST * SA * RG * SA * ST * SGeon001Aeon001Geon001Geo * | | SRSSS nXnXnXS |
| | SGeo | | |
| WV- | mG * SmU * SmG * SmC * SmA * Sm5C * SA * Sm5C * SA * SG | GUGCACACAGTAGATGAGGG | SSSSS SSSSS |
| 23045 | * ST * SA * SG * RA * ST * SGeon001Aeon001Geon001Geo * | | SSRSS nXnXnXS |
| | SGeo | | |
| WV- | m5Ceo * SAeon001m5CeoAeon001Geo * RT * RA * SG * SA * | CACAGTAGATGAGGGAGCAG | SnXOnXR RSSSS |
| 23046 | ST * SG * SA * SG * SG * SG * SAeon001Geom5Ceon001Aeo * | | SSSSS nXOnXS |
| | SGeo | | |
| WV- | m5Ceo * SAeon001m5CeoAeon001Geo * RT * SA * RG * SA * | CACAGTAGATGAGGGAGCAG | SnXOnXR SRSSS |
| 23047 | ST * SG * SA * SG * SG * SG * SAeon001Geom5Ceon001Aeo * | | SSSSS nXOnXS |
| | SGeo | | |
| WV- | m5Ceo * SAeon001m5CeoAeon001Geo * RT * SA * SG * RA * | CACAGTAGATGAGGGAGCAG | SnXOnXR SSRSS |
| 23048 | ST * SG * SA * SG * SG * SG * SAeon001Geom5Ceon001Aeo * | | SSSSS nXOnXS |
| | SGeo | | |
| WV- | Aeo * Sm5Ceon001Aeom5Ceon001Aeo * RG * ST * RA * SG * | ACACAGTAGATGAGGGAGCA | SnXOnXR SRSSS |
| 23049 | SA * ST * SG * SA * SG * SG * SGeon001AeoGeon001m5Ceo * | | SSSSS nXOnXS |
| | SAeo | | |
| WV- | Aeo * Sm5Ceon001Aeom5Ceon001Aeo * RG * ST * SA * RG * | ACACAGTAGATGAGGGAGCA | SnXOnXR SSRSS |
| 23050 | SA * ST * SG * SA * SG * SG * SGeon001AeoGeon001m5Ceo * | | SSSSS nXOnXS |
| | SAeo | | |
| WV- | Aeo * Sm5Ceon001Aeom5Ceon001Aeo * RG * ST * SA * SG * | ACACAGTAGATGAGGGAGCA | SnXOnXR SSSRS |
| 23051 | RA * ST * SG * SA * SG * SG * SGeon001AeoGeon001m5Ceo * | | SSSSS nXOnXS |
| | SAeo | | |
| WV- | m5Ceo * SAeon001m5CeoAeon001m5Ceo * RA * SG * ST * RA * | CACACAGTAGATGAGGGAGC | SnXOnXR SSRSS |
| 23052 | SG * SA * ST * SG * SA * SG * SGeon001GeoAeon001Geo * | | SSSSS nXOnXS |
| | Sm5Ceo | | |
| WV- | m5Ceo * SAeon001m5CeoAeon001m5Ceo * RA * SG * ST * SA * | CACACAGTAGATGAGGGAGC | SnXOnXR SSSRS |
| 23053 | RG * SA * ST * SG * SA * SG * SGeon001GeoAeon001Geo * | | SSSSS nXOnXS |
| | Sm5Ceo | | |
| WV- | m5Ceo * SAeon001m5CeoAeon001m5Ceo * RA * SG * ST * SA * | CACACAGTAGATGAGGGAGC | SnXOnXR SSSSR |
| 23054 | SG * RA * ST * SG * SA * SG * SGeon001GeoAeon001Geo * | | SSSSS nXOnXS |
| | Sm5Ceo | | |
| WV- | Geo * Sm5Ceon001Aeom5Ceon001Aeo * Rm5C * SA * SG * ST * | GCACACAGTAGATGAGGGAG | SnXOnXR SSSRS |
| 23055 | RA * SG * SA * ST * SG * SA * SGeon001GeoGeon001Aeo * | | SSSSS nXOnXS |
| | SGeo | | |
| WV- | Geo * Sm5Ceon001Aeom5Ceon001Aeo * Rm5C * SA * SG * ST * | GCACACAGTAGATGAGGGAG | SnXOnXR SSSSR |
| 23056 | SA * RG * SA * ST * SG * SA * SGeon001GeoGeon001Aeo * | | SSSSS nXOnXS |
| | SGeo | | |
| WV- | Geo * Sm5Ceon001Aeom5Ceon001Aeo * Rm5C * SA * SG * ST * | GCACACAGTAGATGAGGGAG | SnXOnXR SSSSS |
| 23057 | SA * SG * RA * ST * SG * SA * SGeon001GeoGeon001Aeo * | | RSSSS nXOnXS |
| | SGeo | | |
| WV- | Teo * SGeon001m5CeoAeon001m5Ceo * RA * Sm5C * SA * SG * | TGCACACAGTAGATGAGGGA | SnXOnXR SSSSR |
| 23058 | ST * RA * SG * SA * ST * SG * 5Aeon001GeoGeon001Geo * | | SSSSS nXOnXS |
| | SAeo | | |
| WV- | Teo * SGeon001m5CeoAeon001m5Ceo * RA * Sm5C * SA * SG * | TGCACACAGTAGATGAGGGA | SnXOnXR SSSSS |
| 23059 | ST * SA * RG * SA * ST * SG * SAeon001GeoGeon001Geo * | | RSSSS nXOnXS |
| | SAeo | | |
| WV- | Teo * SGeon001m5CeoAeon001m5Ceo * RA * Sm5C * SA * SG * | TGCACACAGTAGATGAGGGA | SnXOnXR SSSSS |
| 23060 | ST * SA * SG * RA * ST * SG * SAeon001GeoGeon001Geo * | | SRSSS nXOnXS |
| | SAeo | | |
| WV- | Geo * STeon001Geom5Ceon001Aeo * Rm5C * SA * Sm5C * SA * | GTGCACACAGTAGATGAGGG | SnXOnXR SSSSS |
| 23061 | SG * ST * RA * SG * SA * ST * SGeon001AeoGeon001Geo * | | RSSSS nXOnXS |
| | SGeo | | |
| WV- | Geo * STeon001Geom5Ceon001Aeo * Rm5C * SA * Sm5C * SA * | GTGCACACAGTAGATGAGGG | SnXOnXR SSSSS |
| 23062 | SG * ST * SA * RG * SA * ST * SGeon001AeoGeon001Geo * | | SRSSS nXOnXS |
| | SGeo | | |
| WV- | Geo * STeon001Geom5Ceon001Aeo * Rm5C * SA * Sm5C * SA * | GTGCACACAGTAGATGAGGG | SnXOnXR SSSSS |
| 23063 | SG * ST * SA * SG * RA * ST * SGeon001AeoGeon001Geo * | | SSRSS nXOnXS |
| | SGeo | | |
| WV- | m5Ceo * SAeon001m5CeoAeon001Geo * RT * RA * SG * SA * | CACAGTAGATGAGGGAGCAG | SnXOnXR RSSSS |
| 23064 | ST * SG * SA * SG * SG * SG * SmAn001mG * SmCn001mA * | | SSSSS nXSnXS |
| | SmG | | |
| WV- | m5Ceo * SAeon001m5CeoAeon001Geo * RT * SA * RG * SA * | CACAGTAGATGAGGGAGCAG | SnXOnXR SRSSS |
| 23065 | ST * SG * SA * SG * SG * SG * SmAn001mG * SmCn001mA * | | SSSSS nXSnXS |
| | SmG | | |
| WV- | m5Ceo * SAeon001m5CeoAeon001Geo * RT * SA * SG * RA * | CACAGTAGATGAGGGAGCAG | SnXOnXR SSRSS |
| 23066 | ST * SG * SA * SG * SG * SG * SmAn001mG * SmCn001mA * | | SSSSS nXSnXS |
| | SmG | | |
| WV- | Aeo * Sm5Ceon001Aeom5Ceon001Aeo * RG * ST * RA * SG * | ACACAGTAGATGAGGGAGCA | SnXOnXR SRSSS |
| 23067 | SA * ST * SG * SA * SG * SG * SmGn001mA * SmGn001mC * | | SSSSS nXSnXS |
| | SmA | | |
| WV- | Aeo * Sm5Ceon001Aeom5Ceon001Aeo * RG * ST * SA * RG * | ACACAGTAGATGAGGGAGCA | SnXOnXR SSRSS |
| 23068 | SA * ST * SG * SA * SG * SG * SmGn001mA * SmGn001mC * | | SSSSS nXSnXS |
| | SmA | | |
| WV- | Aeo * Sm5Ceon001Aeom5Ceon001Aeo * RG * ST * SA * SG * | ACACAGTAGATGAGGGAGCA | SnXOnXR SSSRS |
| 23069 | RA * ST * SG * SA * SG * SG * SmGn001mA * SmGn001mC * | | SSSSS nXSnXS |
| | SmA | | |
| WV- | m5Ceo * SAeon001m5CeoAeon001m5Ceo * RA * SG * ST * RA * | CACACAGTAGATGAGGGAGC | SnXOnXR SSRSS |
| 23070 | SG * SA * ST * SG * SA * SG * SmGn001mG * SmAn001mG * | | SSSSS nXSnXS |
| | SmC | | |
| WV- | m5Ceo * SAeon001m5CeoAeon001m5Ceo * RA * SG * ST * SA * | CACACAGTAGATGAGGGAGC | SnXOnXR SSSRS |
| 23071 | RG * SA * ST * SG * SA * SG * SmGn001mG * SmAn001mG * | | SSSSS nXSnXS |
| | SmC | | |
| WV- | m5Ceo * SAeon001m5CeoAeon001m5Ceo * RA * SG * ST * SA * | CACACAGTAGATGAGGGAGC | SnXOnXR SSSSR |
| 23072 | SG * RA * ST * SG * SA * SG * SmGn001mG * SmAn001mG * | | SSSSS nXSnXS |
| | SmC | | |
| WV- | Geo * Sm5Ceon001Aeom5Ceon001Aeo * Rm5C * SA * SG * ST * | GCACACAGTAGATGAGGGAG | SnXOnXR SSSRS |
| 23073 | RA * SG * SA * ST * SG * SA * SmGn001mG * SmGn001mA * | | SSSSS nXSnXS |
| | SmG | | |
| WV- | Geo * Sm5Ceon001Aeom5Ceon001Aeo * Rm5C * SA * SG * ST * | GCACACAGTAGATGAGGGAG | SnXOnXR SSSSR |
| 23074 | SA * RG * SA * ST * SG * SA * SmGn001mG * SmGn001mA * | | SSSSS nXSnXS |
| | SmG | | |
| WV- | Geo * Sm5Ceon001Aeom5Ceon001Aeo * Rm5C * SA * SG * ST * | GCACACAGTAGATGAGGGAG | SnXOnXR SSSSS |
| 23075 | SA * SG * RA * ST * SG * SA * SmGn001mG * SmGn001mA * | | RSSSS nXSnXS |
| | SmG | | |
| WV- | Teo * SGeon001m5CeoAeon001m5Ceo * RA * Sm5C * SA * SG * | TGCACACAGTAGATGAGGGA | SnXOnXR SSSSR |
| 23076 | ST * RA * SG * SA * ST * SG * SmAn001mG * SmGn001mG * | | SSSSS nXSnXS |
| | SmA | | |
| WV- | Teo * SGeon001m5CeoAeon001m5Ceo * RA * Sm5C * SA * SG * | TGCACACAGTAGATGAGGGA | SnXOnXR SSSSS |
| 23077 | ST * SA * RG * SA * ST * SG * SmAn001mG * SmGn001mG * | | RSSSS nXSnXS |
| | SmA | | |
| WV- | Teo * SGeon001m5CeoAeon001m5Ceo * RA * Sm5C * SA * SG * | TGCACACAGTAGATGAGGGA | SnXOnXR SSSSS |
| 23078 | ST * SA * SG * RA * ST * SG * SmAn001mG * SmGn001mG * | | SRSSS nXSnXS |
| | SmA | | |
| WV- | Geo * STeon001Geom5Ceon001Aeo * Rm5C * SA * Sm5C * SA * | GTGCACACAGTAGATGAGGG | SnXOnXR SSSSS |
| 23079 | SG * ST * RA * SG * SA * ST * SmGn001mA * SmGn001mG * | | RSSSS nXSnXS |
| | SmG | | |
| WV- | Geo * STeon001Geom5Ceon001Aeo * Rm5C * SA * Sm5C * SA * | GTGCACACAGTAGATGAGGG | SnXOnXR SSSSS |
| 23080 | SG * ST * SA * RG * SA * ST * SmGn001mA * SmGn001mG * | | SRSSS nXSnXS |
| | SmG | | |
| WV- | Geo * STeon001Geom5Ceon001Aeo * Rm5C * SA * Sm5C * SA * | GTGCACACAGTAGATGAGGG | SnXOnXR SSSSS |
| 23081 | SG * ST * SA * SG * RA * ST * SmGn001mA * SmGn001mG * | | SSRSS nXSnXS |
| | SmG | | |
| WV- | mC * SmAn001mC * SmAn001mG * ST * RA * SG * SA * ST * | CACAGTAGATGAGGGAGCAG | SnXSnXS RSSSS |
| 23082 | SG * SA * SG * SG * SG * SAeon001Geom5Ceon001Aeo * SGeo | | SSSSS nXOnXS |
| WV- | mC * SmAn001mC * SmAn001mG * ST * SA * RG * SA * ST * | CACAGTAGATGAGGGAGCAG | SnXSnXS SRSSS |
| 23083 | SG * SA * SG * SG * SG * SAeon001Geom5Ceon001Aeo * SGeo | | SSSSS nXOnXS |
| WV- | mC * SmAn001mC * SmAn001mG * ST * SA * SG * RA * ST * | CACAGTAGATGAGGGAGCAG | SnXSnXS SSRSS |
| 23084 | SG * SA * SG * SG * SG * SAeon001Geom5Ceon001Aeo * SGeo | | SSSSS nXOnXS |
| WV- | mA * SmCn001mA * SmCn001mA * SG * ST * RA * SG * SA * | ACACAGTAGATGAGGGAGCA | SnXSnXS SRSSS |
| 23085 | ST * SG * SA * SG * SG * SGeon001AeoGeon001m5Ceo * SAeo | | SSSSS nXOnXS |
| WV- | mA * SmCn001mA * SmCn001mA * SG * ST * SA * RG * SA * | ACACAGTAGATGAGGGAGCA | SnXSnXS SSRSS |
| 23086 | ST * SG * SA * SG * SG * SGeon001AeoGeon001m5Ceo * SAeo | | SSSSS nXOnXS |
| WV- | mA * SmCn001mA * SmCn001mA * SG * ST * SA * SG * RA * | ACACAGTAGATGAGGGAGCA | SnXSnXS SSSRS |
| 23087 | ST * SG * SA * SG * SG * SGeon001AeoGeon001m5Ceo * SAeo | | SSSSS nXOnXS |
| WV- | mC * SmAn001mC * SmAn001mC * SA * SG * ST * RA * SG * | CACACAGTAGATGAGGGAGC | SnXSnXS SSRSS |
| 23088 | SA * ST * SG * SA * SG * SGeon001GeoAeon001Geo * Sm5Ceo | | SSSSS nXOnXS |
| WV- | mC * SmAn001mC * SmAn001mC * SA * SG * ST * SA * RG * | CACACAGTAGATGAGGGAGC | SnXSnXS SSSRS |
| 23089 | SA * ST * SG * SA * SG * SGeon001GeoAeon001Geo * Sm5Ceo | | SSSSS nXOnXS |
| WV- | mC * SmAn001mC * SmAn001mC * SA * SG * ST * SA * SG * | CACACAGTAGATGAGGGAGC | SnXSnXS SSSSR |
| 23090 | RA * ST * SG * SA * SG * SGeon001GeoAeon001Geo * Sm5Ceo | | SSSSS nXOnXS |
| WV- | mG * SmCn001mA * SmCn001mA * Sm5C * SA * SG * ST * RA | GCACACAGTAGATGAGGGAG | SnXSnXS SSSRS |
| 23091 | * SG * SA * ST * SG * SA * SGeon001GeoGeon001Aeo * SGeo | | SSSSS nXOnXS |
| WV- | mG * SmCn001mA * SmCn001mA * Sm5C * SA * SG * ST * SA | GCACACAGTAGATGAGGGAG | SnXSnXS SSSSR |
| 23092 | * RG * SA * ST * SG * SA * SGeon001GeoGeon001Aeo * SGeo | | SSSSS nXOnXS |
| WV- | mG * SmCn001mA * SmCn001mA * Sm5C * SA * SG * ST * SA | GCACACAGTAGATGAGGGAG | SnXSnXS SSSSS |
| 23093 | * SG * RA * ST * SG * SA * SGeon001GeoGeon001Aeo * SGeo | | RSSSS nXOnXS |
| WV- | mU * SmGn001mC * SmAn001mC * SA * Sm5C * SA * SG * ST | UGCACACAGTAGATGAGGGA | SnXSnXS SSSSR |
| 23094 | * RA * SG * SA * ST * SG * 5Aeon001GeoGeon001Geo * SAeo | | SSSSS nXOnXS |
| WV- | mU * SmGn001mC * SmAn001mC * SA * Sm5C * SA * SG * ST | UGCACACAGTAGATGAGGGA | SnXSnXS SSSSS |
| 23095 | * SA * RG * SA * ST * SG * SAeon001GeoGeon001Geo * SAeo | | RSSSS nXOnXS |
| WV- | mU * SmGn001mC * SmAn001mC * SA * Sm5C * SA * SG * ST | UGCACACAGTAGATGAGGGA | SnXSnXS SSSSS |
| 23096 | * SA * SG * RA * ST * SG * SAeon001GeoGeon001Geo * SAeo | | SRSSS nXOnXS |
| WV- | mG * SmUn001mG * SmCn001mA * Sm5C * SA * Sm5C * SA * | GUGCACACAGTAGATGAGGG | SnXSnXS SSSSS |
| 23097 | SG * ST * RA * SG * SA * ST * SGeon001AeoGeon001Geo * | | RSSSS nXOnXS |
| | SGeo | | |
| WV- | mG * SmUn001mG * SmCn001mA * Sm5C * SA * Sm5C * SA * | GUGCACACAGTAGATGAGGG | SnXSnXS SSSSS |
| 23098 | SG * ST * SA * RG * SA * ST * SGeon001AeoGeon001Geo * | | SRSSS nXOnXS |
| | SGeo | | |
| WV- | mG * SmUn001mG * SmCn001mA * Sm5C * SA * Sm5C * SA * | GUGCACACAGTAGATGAGGG | SnXSnXS SSSSS |
| 23099 | SG * ST * SA * SG * RA * ST * SGeon001AeoGeon001Geo * | | SSRSS nXOnXS |
| | SGeo | | |
| WV- | m5Ceo * SAeom5CeoAeoGeo * RT * SA * SG * SA * ST * SG * | CACAGTAGATGAGGGAGCAG | SOOOR SSSSS |
| 23100 | SA * SG * SG * SG * SAeoGeom5CeoAeo * SGeo | | SSSSS OOOS |
| WV- | m5Ceo * SAeom5CeoAeoGeo * RT * SA * SG * SA * ST * SG * | CACAGTAGATGAGGGAGCAG | SOOOR SSSSS |
| 23101 | SA * SG * SG * SG * SmA * SmG * SmC * SmA * SmG | | SSSSS SSSS |
| WV- | mC * SmA * SmC * SmA * SmG * ST * SA * SG * SA * ST * SG | CACAGTAGATGAGGGAGCAG | SSSSS SSSSS |
| 23102 | * SA * SG * SG * SG * SAeoGeom5CeoAeo * SGeo | | SSSSS OOOS |
| WV- | m5Ceo * SAeon001m5Ceon001Aeon001Geo * RT * SA * SG * SA | CACAGTAGATGAGGGAGCAG | SnXnXnXR SSSSS |
| 23103 | * ST * SG * SA * SG * SG * SG * SAeoGeom5CeoAeo * SGeo | | SSSSS OOOS |
| WV- | m5Ceo * SAeom5CeoAeoGeo * RT * SA * SG * SA * ST * SG * | CACAGTAGATGAGGGAGCAG | SOOOR SSSSS |
| 23104 | SA * SG * SG * SG * SAeon001Geon001m5Ceon001Aeo * SGeo | | SSSSS nXnXnXS |
| WV- | m5Ceo * SAeon001m5Ceon001Aeon001Geo * RT * SA * SG * SA | CACAGTAGATGAGGGAGCAG | SnXnXnXR SSSSS |
| 23105 | * ST * SG * SA * SG * SG * SG * SmA * SmG * SmC * SmA * | | SSSSS SSSS |
| | SmG | | |
| WV- | mC * SmA * SmC * SmA * SmG * ST * SA * SG * SA * ST * SG | CACAGTAGATGAGGGAGCAG | SSSSS SSSSS |
| 23106 | * SA * SG * SG * SG * SAeon001Geon001m5Ceon001Aeo * SGeo | | SSSSS nXnXnXS |
| WV- | m5Ceo * SAeon001m5CeoAeon001Geo * RT * SA * SG * SA * ST | CACAGTAGATGAGGGAGCAG | SnXOnXR SSSSS |
| 23107 | * SG * SA * SG * SG * SG * SAeon001Geom5Ceon001Aeo * | | SSSSS nX0nXS |
| | SGeo | | |
| WV- | m5Ceo * SAeon001m5CeoAeon001Geo * RT * SA * SG * SA * ST | CACAGTAGATGAGGGAGCAG | SnXOnXR SSSSS |
| 23108 | * SG * SA * SG * SG * SG * SmAn001mG * SmCn001mA * SmG | | SSSSS nXSnXS |
| WV- | mC * SmAn001mC * SmAn001mG * ST * SA * SG * SA * ST * | CACAGTAGATGAGGGAGCAG | SnXSnXS SSSSS |
| 23109 | SG * SA * SG * SG * SG * SAeon001Geom5Ceon001Aeo * SGeo | | SSSSS nXOnXS |
| WV- | m5Ceo * Teo * m5Ceo * Aeo * Geo * T * A * A * m5C * A * T * T | CTCAGTAACATTGACACCAC | XXXXX XXXXX |
| 23374 | * G * A * m5C * Aeo * m5Ceo * m5Ceo * Aeo * m5Ceo | | XXXXX XXXX |
| WV- | m5Ceo * Teo * m5Ceo * Geo * Aeo * m5C * T * A * A * A * G * | CTCGACTAAAGCAGGATTTC | XXXXX XXXXX |
| 23375 | m5C * A * G * G * Aeo * Teo * Teo * Teo * m5Ceo | | XXXXX XXXX |
| WV- | Geo * m5Ceo * Aeo * Geo * Geo * G * T * T * A * m5C * m5C * | GCAGGGTTACCGCCATCCCC | XXXXX XXXXX |
| 23376 | G * m5C * m5C * A * Teo * m5Ceo * m5Ceo * m5Ceo * m5Ceo | | XXXXX XXXX |
| WV- | m5Ceo * Geo * Aeo * Geo * Aeo * m5C * A * G * T * m5C * G * | CGAGACAGTCGCTTCCACTT | XXXXX XXXXX |
| 23377 | m5C * T * T * m5C * m5Ceo * Aeo * m5Ceo * Teo * Teo | | XXXXX XXXX |
| WV- | A * RC * SA * SC * SA * SG * ST * SA * SG * RA * ST * | ACACAGTAGATGAGGG | RSSSS SSSRS S |
| 23463 | SmGmAmGmG * SmG | | OOOS |
| WV- | C * RA * SC * SA * SG * ST * SA * SG * RA * ST * | CACAGTAGATGAGGG | RSSSS SSRSS |
| 23464 | SmGmAmGmG * SmG | | OOOS |
| WV- | A * RC * SA * SG * ST * SA * SG * RA * ST * SmGmAmGmG * | ACAGTAGATGAGGG | RSSSS SRSS |
| 23465 | SmG | | OOOS |
| WV- | C * RA * SG * ST * SA * SG * RA * ST * SmGmAmGmG * SmG | CAGTAGATGAGGG | RSSSS RSS OOOS |
| 23466 | | | |
| WV-23467 | A * RG * ST * SA * SG * RA * ST * SmGmAmGmG * SmG | AGTAGATGAGGG | RSSSRSS OOOS |
| WV-23468 | G * RT * SA * SG * RA * ST * SmGmAmGmG * SmG | GTAGATGAGGG | RSSRSS OOOS |
| WV-23469 | T * RA * SG * RA * ST * SmGmAmGmG * SmG | TAGATGAGGG | RSRSS OOOS |
| WV-23470 | A * RG * RA * ST * SmGmAmGmG * SmG | AGATGAGGG | RRSS OOOS |
| WV-23471 | G * SA * ST * SmGmAmGmG * SmG | GATGAGGG | SSS OOOS |
| WV-23472 | A * RT * SmGmAmGmG * SmG | ATGAGGG | RS OOOS |
| WV-23473 | T * RmGmAmGmG * SmG | TGAGGG | R OOOS |
| WV-23474 | A * SC * SA * SC * SA * SG * ST * SA * SG * RA * ST * | ACACAGTAGATGAGGG | SSSSS SSSRS S |
| | SmGmAmGmG * SmG | | OOOS |
| WV-23475 | C * SA * SC * SA * SG * ST * SA * SG * RA * ST * | CACAGTAGATGAGGG | SSSSS SSRSS |
| | SmGmAmGmG * SmG | | OOOS |
| WV-23476 | A * SC * SA * SG * ST * SA * SG * RA * ST * SmGmAmGmG * | ACAGTAGATGAGGG | SSSSS SRSS OOOS |
| | SmG | | |
| WV-23477 | C * SA * SG * ST * SA * SG * RA * ST * SmGmAmGmG * SmG | CAGTAGATGAGGG | SSSSS RSS OOOS |
| WV-23478 | A * SG * ST * SA * SG * RA * ST * SmGmAmGmG * SmG | AGTAGATGAGGG | SSSSRSS OOOS |
| WV-23479 | G * ST * SA * SG * RA * ST * SmGmAmGmG * SmG | GTAGATGAGGG | SSSRSS OOOS |
| WV-23480 | T * SA * SG * RA * ST * SmGmAmGmG * SmG | TAGATGAGGG | SSRSS OOOS |
| WV-23481 | A * SG * RA * ST * SmGmAmGmG * SmG | AGATGAGGG | SRSS OOOS |
| WV-23482 | G * RA * ST * SmGmAmGmG * SmG | GATGAGGG | RSS OOOS |
| WV-23483 | A * ST * SmGmAmGmG * SmG | ATGAGGG | SS OOOS |
| WV-23484 | T * SmGmAmGmG * SmG | TGAGGG | SOOOS |
| WV- | L001m5Ceo * Teom5CeoGeoAeo * m5C *T * A * A * A *G * | CTCGACTAAAGCAGGATTTC | OXOOO XXXXX |
| 23572 | m5C * A * G * G * AeoTeoTeoTeo * m5Ceo | | XXXXX XOOOX |
| WV- | Mod001L001m5Ceo * Teom5CeoGeoAeo * m5C *T * A * A * A * | CTCGACTAAAGCAGGATTTC | OXOOO XXXXX |
| 23573 | G * m5C * A * G * G * AeoTeoTeoTeo * m5Ceo | | XXXXX XOOOX |
| WV- | L001m5Ceo * Teom5CeoAeoGeo * T * A * A * C * A * T * T * G | CTCAGTAACATTGACACCAC | OXOOO XXXXX |
| 23574 | * A * C * Aeom5Ceom5CeoAeo * m5Ceo | | XXXXX XOOOX |
| WV- | Mod001L001m5Ceo * Teom5CeoAeoGeo * T * A * A * C * A * T | CTCAGTAACATTGACACCAC | OXOOO XXXXX |
| 23575 | * T * G * A * C * Aeom5Ceom5CeoAeo * m5Ceo | | XXXXX XOOOX |
| WV- | mG * SmUn001RmU * SmGn001RmA * ST * Sm5C * ST * SG * | GUUGATCTGTAGCAGCAGCT | SnRSnRS SSSSR |
| 23689 | ST * RA * SG * Sm5C * SA * SG * | | SS SSR nROnRS |
| | Rm5Ceon001RAeoGeon001Rm5Ceo * STeo | | |
| WV- | mG * SmUn001RmUmGn001RmA * ST * Sm5C * ST * SG * ST * | GUUGATCTGTAGCAGCAGCT | SnROnRS SSSSR |
| 23690 | RA * SG * Sm5C * SA * SG * | | SS SSR nROnRS |
| | Rm5Ceon001RAeoGeon001Rm5Ceo * STeo | | |
| WV- | mG * SmUn001RmUmGn001RmA * ST * Sm5C * ST * SG * ST * | GUUGATCTGTAGCAGCAGCT | SnROnRS SSSSR |
| 23691 | RA * SG * Sm5C * SA * SG * Rm5CeoAeoGeon001Rm5Ceo * | | SSSSR OOnRS |
| | STeo | | |
| WV- | mG * SmUn001RmU * SmGmA * ST * Sm5C * ST * SG * ST * | GUUGATCTGTAGCAGCAGCT | SnRSOS SSSSR |
| 23692 | RA * SG * Sm5C * SA * SG * | | SS SSR nROnRS |
| | Rm5Ceon001RAeoGeon001Rm5Ceo * STeo | | |
| WV- | mG * SmGmCmAA * SA * SG * SG * SG * SC * SA * SC * RA * | GGCAAAGGGCACAGACUUC | SOOOS SSSSS |
| 23693 | SG * SmAmCmUmU * SmC | | SRSS OOOS |
| WV- | mG * SmGmCmAmC * SA * SA * SG * SG * SG * SC * SA * SC | GGCACAAGGGCACAGACUC | SOOOS SSSSS |
| 23694 | * RA * SG * SmAmCmUmC | | SSRSS OOO |
| WV- | mG * SmGmAmC * SA * SA * SG * SG * SG * SC * SA * SC * | GGACAAGGGCACAGACUUC | SOOSS SSSSS |
| 23695 | RA * SG * SmAmCmUmU * SmC | | SRSS OOOS |
| WV- | mG * SmGmCmAmC * SA * SA * SG * SG * SG * SC * SA * SC | GGCACAAGGGCACAGAUUC | SOOOS SSSSS |
| 23696 | * RA * SG * SmAmUmU * SmC | | SSRSS OOS |
| WV- | mG * SmGmCmAmC * SA * SA * SG * SG * SG * SC * SC * SA | GGCACAAGGGCCACAGACUUC | SOOOS SSSSS |
| 23697 | * SC * RA * SG * SmAmCmUmU * SmC | | SSSRS S OOOS |
| WV- | mG * SmGmCmAmC * SA * SA * SG * SG * SG * SC * SA * SC | GGCACAAGGGCACCAGACUUC | SOOOS SSSSS |
| 23698 | * RC * RA * SG * SmAmCmUmU * SmC | | SSRRSS OOOS |
| WV- | mG * SmGmCmAmC * SA * SA * SG * SG * SG * SC * SA * SA | GGCACAAGGGCAACAGACUUC | SOOOS SSSSS |
| 23699 | * SC * RA * SG * SmAmCmUmU * SmC | | SSSRS S OOOS |
| WV- | mG * SmGmCmAmC * SA * SA * SG * SG * SG * SC * SA * SC | GGCACAAGGGCACAAGACUUC | SOOOS SSSSS |
| 23700 | * RA * SA * SG * SmAmCmUmU * SmC | | SSRSS S OOOS |
| WV- | mG * SmGmCmAmC * SmC * SA * SA * SG * SG * SG * SC * | GGCACCAAGGGCACAGACUUC | SOOOS SSSSS |
| 23701 | RA * SC * SA * SG * SmAmCmUmU * SmC | | SRSSS S OOOS |
| WV- | mG * SmGmCmAmCA * SA * SG * SG * SG * SC * SA * SC * | GGCACAAGGGCACAGACUUC | SOOOO SSSSS |
| 23702 | RA * SG * SmAmCmUmU * SmC | | SSRSS OOOS |
| WV- | mG * SmGmCmAmC * SAA * SG * SG * SG * SC * SA * SC * | GGCACAAGGGCACAGACUUC | SOOOS OSSSS |
| 23703 | RA * SG * SmAmCmUmU * SmC | | SSRSS OOOS |
| WV- | mG * SmGmCmAmC * SA * SAG * SG * SG * SC * SA * SC * | GGCACAAGGGCACAGACUUC | SOOOS SOSSS |
| 23704 | RA * SG * SmAmCmUmU * SmC | | SSRSS OOOS |
| WV- | mG * SmGmCmAmC * SA * SA * SGG * SG * SC * SA * SC * | GGCACAAGGGCACAGACUUC | SOOOS SSOSS |
| 23705 | RA * SG * SmAmCmUmU * SmC | | SSRSS OOOS |
| WV- | mG * SmGmCmAmC * SA * SA * SG * SGG * SC * SA * SC * | GGCACAAGGGCACAGACUUC | SOOOS SSSOS |
| 23706 | RA * SG * SmAmCmUmU * SmC | | SSRSS OOOS |
| WV- | mG * SmGmCmAmC * SA * SA * SG * SG * SGC * SA * SC * | GGCACAAGGGCACAGACUUC | SOOOS SSSSO |
| 23707 | RA * SG * SmAmCmUmU * SmC | | SSRSS OOOS |
| WV- | mG * SmGmCmAmC * SA * SA * SG * SG * SG * SCA * SC * | GGCACAAGGGCACAGACUUC | SOOOS SSSSS |
| 23708 | RA * SG * SmAmCmUmU * SmC | | OSRSS OOOS |
| WV- | mG * SmGmCmAmC * SA * SA * SG * SG * SG * SC * SAC * | GGCACAAGGGCACAGACUUC | SOOOS SSSSS |
| 23709 | RA * SG * SmAmCmUmU * SmC | | SORSS OOOS |
| WV- | mG * SmGmCmAmC * SA * SA * SG * SG * SG * SC * SA * | GGCACAAGGGCACAGACUUC | SOOOS SSSSS |
| 23710 | SCA * SG * SmAmCmUmU * SmC | | SSOSS OOOS |
| WV- | mG * SmGmCmAmC * SA * SA * SG * SG * SG * SC * SA * SC | GGCACAAGGGCACAGACUUC | SOOOS SSSSS |
| 23711 | * RAG * SmAmCmUmU * SmC | | SSROS OOOS |
| WV- | mG * SmGmCmAmC * SA * SA * SG * SG * SG * SC * SA * SC | GGCACAAGGGCACAGACUUC | SOOOS SSSSS |
| 23712 | * RA * SGmAmCmUmU * SmC | | SSRSO OOOS |
| WV- | mG * SmGmCmAmC * SA * SA * SG * SG * SG * SC * SA * SC | GGCACAAGGGCACAGACUUC | SOOOS SSSSS |
| 23713 | * RA * SG * SmAmCmUmUmC | | SSRSS OOOO |
| WV- | dmtrmG * RmUmGmCmA * SC * SA * SC * SA * SG * ST * SA * | GUGCACACAGTAGATGAGGG | ROOOS SSSSS |
| 23714 | SG * RA * ST * SmGmAmGmG * SmG | | SSRSS OOOS |
| WV- | dmtrdA * RC * SA * SC * SA * SG * ST * SA * SG * RA * ST * | ACACAGTAGATGAGGG | RSSSS SSSRS S |
| 23715 | SmGmAmGmG * SmG | | OOOS |
| WV- | dmtrdC * RA * SC * SA * SG * ST * SA * SG * RA * ST * | CACAGTAGATGAGGG | RSSSS SSRSS |
| 23716 | SmGmAmGmG * SmG | | OOOS |
| WV- | dmtrdA * RC * SA * SG * ST * SA * SG * RA * ST * | ACAGTAGATGAGGG | RSSSS SRSS |
| 23717 | SmGmAmGmG * SmG | | OOOS |
| WV- | dmtrdC * RA * SG * ST * SA * SG * RA * ST * SmGmAmGmG * | CAGTAGATGAGGG | RSSSS RSS OOOS |
| 23718 | SmG | | |
| WV- | dmtrdA * RG * ST * SA * SG * RA * ST * SmGmAmGmG * SmG | AGTAGATGAGGG | RSSSRSS OOOS |
| 23719 | | | |
| WV- | dmtrdG * RT * SA * SG * RA * ST * SmGmAmGmG * SmG | GTAGATGAGGG | RSSRSS OOOS |
| 23720 | | | |
| WV- | dmtrdT * RA * SG * RA * ST * SmGmAmGmG * SmG | TAGATGAGGG | RSRSS OOOS |
| 23721 | | | |
| WV- | dmtrdA * RG * RA * ST * SmGmAmGmG * SmG | AGATGAGGG | RRSS OOOS |
| 23722 | | | |
| WV- | dmtrdG * SA * ST * SmGmAmGmG * SmG | GATGAGGG | SSS OOOS |
| 23723 | | | |
| WV- | dmtrdA * RT * SmGmAmGmG * SmG | ATGAGGG | RS OOOS |
| 23724 | | | |
| WV- | dmtrdT * RmGmAmGmG * SmG | TGAGGG | R OOOS |
| 23725 | | | |
| WV- | dmtrmG * RmG | GG | R |
| 23726 | | | |
| WV- | dmtrmG * SmUmGmCmA * SC * SA * SC * SA * SG * ST * SA * | GUGCACACAGTAGATGAGGG | SOOOS SSSSS |
| 23727 | SG * RA * ST * SmGmAmGmG * SmG | | SSRSS OOOS |
| WV- | dmtrdA * SC * SA * SC * SA * SG * ST * SA * SG * RA * ST * | ACACAGTAGATGAGGG | SSSSS SSSRS S |
| 23728 | SmGmAmGmG * SmG | | OOOS |
| WV- | dmtrdC * SA * SC * SA * SG * ST * SA * SG * RA * ST * | CACAGTAGATGAGGG | SSSSS SSRSS |
| 23729 | SmGmAmGmG * SmG | | OOOS |
| WV- | dmtrdA * SC * SA * SG * ST * SA * SG * RA * ST * | ACAGTAGATGAGGG | SSSSS SRSS OOOS |
| 23730 | SmGmAmGmG * SmG | | |
| WV- | dmtrdC * SA * SG * ST * SA * SG * RA * ST * SmGmAmGmG * | CAGTAGATGAGGG | SSSSS RSS OOOS |
| 23731 | SmG | | |
| WV- | dmtrdA * SG * ST * SA * SG * RA * ST * SmGmAmGmG * SmG | AGTAGATGAGGG | SSSSR SS OOOS |
| 23732 | | | |
| WV-23733 | dmtrdG * ST * SA * SG * RA * ST * SmGmAmGmG * SmG | GTAGATGAGGG | SSSRS S OOOS |
| WV-23734 | dmtrdT * SA * SG * RA * ST * SmGmAmGmG * SmG | TAGATGAGGG | SSRSS OOOS |
| WV-23735 | dmtrdA * SG * RA * ST * SmGmAmGmG * SmG | AGATGAGGG | SRSS OOOS |
| WV-23736 | dmtrdG * RA * ST * SmGmAmGmG * SmG | GATGAGGG | RSS OOOS |
| WV-23737 | dmtrdA * ST * SmGmAmGmG * SmG | ATGAGGG | SS OOOS |
| WV-23738 | dmtrdT * SmGmAmGmG * SmG | TGAGGG | SOOOS |
| WV-23739 | dmtrmG * SmG | GG | S |
| WV- | mG * SmUmGmCmA * SC * SA * SA * SC * SA * SG * ST * SA | GUGCACAACAGTAGATGAGGG | SOOOS SSSSS |
| 23772 | * SG * RA * ST * SmGmAmGmG * SmG | | SSSRS S OOOS |
| WV- | mG * SmUmGmCmA * SC * SA * SC * SA * SA * SG * ST * SA | GUGCACACAAGTAGATGAGGG | SOOOS SSSSS |
| 23773 | * SG * RA * ST * SmGmAmGmG * SmG | | SSSRS S OOOS |
| WV- | mG * SmUmGmCmA * SC * SA * SC * SA * SG * ST * SA * SA | GUGCACACAGTAAGATGAGGG | SOOOS SSSSS |
| 23774 | * SG * RA * ST * SmGmAmGmG * SmG | | SSSRS S OOOS |
| WV- | mG * SmUmGmCmA * SC * SA * SC * SA * SG * ST * SA * SG | GUGCACACAGTAGAATGAGGG | SOOOS SSSSS |
| 23775 | * RA * SA * ST * SmGmAmGmG * SmG | | SSRSS S OOOS |
| WV- | mG * SmUmGmCmA * SC * SC * SA * SC * SA * SG * ST * SA | GUGCACCACAGTAGATGAGGG | SOOOS SSSSS |
| 23776 | * SG * RA * ST * SmGmAmGmG * SmG | | SSSRS S OOOS |
| WV- | mG * SmUmGmCmA * SC * SA * SC * SC * SA * SG * ST * SA | GUGCACACCAGTAGATGAGGG | SOOOS SSSSS |
| 23777 | * SG * RA * ST * SmGmAmGmG * SmG | | SSSRS S OOOS |
| WV- | mG * SmUmGmCmA * SmA * SC * SA * SC * SA * SG * ST * | GUGCAACACAGTAGATGAGGG | SOOOS SSSSS |
| 23778 | SA * SG * RA * ST * SmGmAmGmG * SmG | | SSSRS S OOOS |
| WV- | mG * SmUmGmCC * SA * SC * SA * SG * ST * SA * SG * RA * | GUGCCACAGTAGATGAGGG | SOOOS SSSSS |
| 23779 | ST * SmGmAmGmG * SmG | | SRSSO OOS |
| WV- | mG * SmUmGmCmA * SC * SC * SA * SG * ST * SA * SG * RA | GUGCACCAGTAGATGAGGG | SOOOS SSSSS |
| 23780 | * ST * SmGmAmGmG * SmG | | SRSSO OOS |
| WV- | mG * SmUmGmCmA * SC * SA * SC * SG * ST * SA * SG * RA | GUGCACACGTAGATGAGGG | SOOOS SSSSS |
| 23781 | * ST * SmGmAmGmG * SmG | | SRSSO OOS |
| WV- | mG * SmUmGmCmA * SC * SA * SC * SA * SG * ST * SG * RA | GUGCACACAGTGATGAGGG | SOOOS SSSSS |
| 23782 | * ST * SmGmAmGmG * SmG | | SRSSO OOS |
| WV- | mG * SmUmGmCmA * SC * SA * SC * SA * SG * ST * SA * SG | GUGCACACAGTAGTGAGGG | SOOOS SSSSS |
| 23783 | * RT * SmGmAmGmG * SmG | | SSRSO OOS |
| WV- | mG * SmUmGmCmA * SA * SC * SA * SG * ST * SA * SG * RA | GUGCAACAGTAGATGAGGG | SOOOS SSSSS |
| 23784 | * ST * SmGmAmGmG * SmG | | SRSSO OOS |
| WV- | mG * SmUmGmCmA * SC * SA * SA * SG * ST * SA * SG * RA | GUGCACAAGTAGATGAGGG | SOOOS SSSSS |
| 23785 | * ST * SmGmAmGmG * SmG | | SRSSO OOS |
| WV- | mA * SC * SA * SC * SA * SG * ST * SA * SG * RA * ST * | ACACAGTAGATGAGGG | SSSSS SSSRS S |
| 23786 | SmGmAmGmG * SmG | | OOOS |
| WV- | mG * SmUmGmCmAC * SA * SC * SA * SG * ST * SA * SG * | GUGCACACAGTAGATGAGGG | SOOOO SSSSS |
| 23787 | RA * ST * SmGmAmGmG * SmG | | SSRSS OOOS |
| WV- | mG * SmUmGmCmA * SCA * SC * SA * SG * ST * SA * SG * | GUGCACACAGTAGATGAGGG | SOOOS OSSSS |
| 23788 | RA * ST * SmGmAmGmG * SmG | | SSRSS OOOS |
| WV- | mG * SmUmGmCmA * SC * SAC * SA * SG * ST * SA * SG * | GUGCACACAGTAGATGAGGG | SOOOS SOSSS |
| 23789 | RA * ST * SmGmAmGmG * SmG | | SSRSS OOOS |
| WV- | mG * SmUmGmCmA * SC * SA * SCA * SG * ST * SA * SG * | GUGCACACAGTAGATGAGGG | SOOOS SSOSS |
| 23790 | RA * ST * SmGmAmGmG * SmG | | SSRSS OOOS |
| WV- | mG * SmUmGmCmA * SC * SA * SC * SAG * ST * SA * SG * | GUGCACACAGTAGATGAGGG | SOOOS SSSOS |
| 23791 | RA * ST * SmGmAmGmG * SmG | | SSRSS OOOS |
| WV- | mG * SmUmGmCmA * SC * SA * SC * SA * SGT * SA * SG * | GUGCACACAGTAGATGAGGG | SOOOS SSSSO |
| 23792 | RA * ST * SmGmAmGmG * SmG | | SSRSS OOOS |
| WV- | mG * SmUmGmCmA * SC * SA * SC * SA * SG * STA * SG * | GUGCACACAGTAGATGAGGG | SOOOS SSSSS |
| 23793 | RA * ST * SmGmAmGmG * SmG | | OSRSS OOOS |
| WV- | mG * SmUmGmCmA * SC * SA * SC * SA * SG * ST * SAG * | GUGCACACAGTAGATGAGGG | SOOOS SSSSS |
| 23794 | RA * ST * SmGmAmGmG * SmG | | SORSS OOOS |
| WV- | mG * SmUmGmCmA * SC * SA * SC * SA * SG * ST * SA * | GUGCACACAGTAGATGAGGG | SOOOS SSSSS |
| 23795 | SGA * ST * SmGmAmGmG * SmG | | SSOSS OOOS |
| WV- | mG * SmUmGmCmA * SC * SA * SC * SA * SG * ST * SA * SG | GUGCACACAGTAGATGAGGG | SOOOS SSSSS |
| 23796 | * RAT * SmGmAmGmG * SmG | | SSROS OOOS |
| WV- | mG * SmUmGmCmA * SC * SA * SC * SA * SG * ST * SA * SG | GUGCACACAGTAGATGAGGG | SOOOS SSSSS |
| 23797 | * RA * STmGmAmGmG * SmG | | SSRSO OOOS |
| WV- | mG * SmUmGmCmA * SC * SA * SC * SA * SG * ST * SA * SG | GUGCACACAGTAGATGAGGG | SOOOS SSSSS |
| 23798 | * RA * ST * SmGmAmGmGmG | | SSRSS OOOO |
| WV- | Geo * SAeoTeom5CeoTeo * RG * RA * SA * SG * SC * SA * SG | GATCTGAAGCAGCAGCTTCT | SOOOR RSSSS |
| 25460 | * SC * SA * SG * Sm5CeoTeoTeom5Ceo * STeo | | SSSSS OOOS |
| WV- | Geo * SAeoTeom5CeoTeo * RG * SA * RA * SG * SC * SA * SG | GATCTGAAGCAGCAGCTTCT | SOOOR SRSSS |
| 25461 | * SC * SA * SG * Sm5CeoTeoTeom5Ceo * STeo | | SSSSS OOOS |
| WV- | Geo * SAeoTeom5CeoTeo * RG * SA * SA * RG * SC * SA * SG | GATCTGAAGCAGCAGCTTCT | SOOOR SSRSS |
| 25462 | * SC * SA * SG * Sm5CeoTeoTeom5Ceo * STeo | | SSSSS OOOS |
| WV- | Teo * SGeoAeoTeom5Ceo * RT * SG * RA * SA * SG * SC * SA * | TGATCTGAAGCAGCAGCTTC | SOOOR SRSSS |
| 25463 | SG * SC * SA * SGeom5CeoTeoTeo * Sm5Ceo | | SSSSS OOOS |
| WV- | Teo * SGeoAeoTeom5Ceo * RT * SG * SA * RA * SG * SC * SA * | TGATCTGAAGCAGCAGCTTC | SOOOR SSRSS |
| 25464 | SG * SC * SA * SGeom5CeoTeoTeo * Sm5Ceo | | SSSSS OOOS |
| WV- | Teo * SGeoAeoTeom5Ceo * RT * SG * SA * SA * RG * SC * SA * | TGATCTGAAGCAGCAGCTTC | SOOOR SSSRS |
| 25465 | SG * SC * SA * SGeom5CeoTeoTeo * Sm5Ceo | | SSSSS OOOS |
| WV- | Teo * STeoGeoAeoTeo * RC * ST * SG * RA * SA * SG * SC * | TTGATCTGAAGCAGCAGCTT | SOOOR SSRSS |
| 25466 | SA * SG * SC * SAeoGeom5CeoTeo * STeo | | SSSSS OOOS |
| WV- | Teo * STeoGeoAeoTeo * RC * ST * SG * SA * RA * SG * SC * | TTGATCTGAAGCAGCAGCTT | SOOOR SSSRS |
| 25467 | SA * SG * SC * SAeoGeom5CeoTeo * STeo | | SSSSS OOOS |
| WV- | Teo * STeoGeoAeoTeo * RC * ST * SG * SA * SA * RG * SC * | TTGATCTGAAGCAGCAGCTT | SOOOR SSSSR |
| 25468 | SA * SG * SC * SAeoGeom5CeoTeo * STeo | | SSSSS OOOS |
| WV- | Geo * STeoTeoGeoAeo * RT * SC * ST * SG * RA * SA * SG * | GTTGATCTGAAGCAGCAGCT | SOOOR SSSRS |
| 25469 | SC * SA * SG * Sm5CeoAeoGeom5Ceo * STeo | | SSSSS OOOS |
| WV- | Geo * STeoTeoGeoAeo * RT * SC * ST * SG * SA * RA * SG * | GTTGATCTGAAGCAGCAGCT | SOOOR SSSSR |
| 25470 | SC * SA * SG * Sm5CeoAeoGeom5Ceo * STeo | | SSSSS OOOS |
| WV- | Geo * STeoTeoGeoAeo * RT * SC * ST * SG * SA * SA * RG * | GTTGATCTGAAGCAGCAGCT | SOOOR SSSSS |
| 25471 | SC * SA * SG * Sm5CeoAeoGeom5Ceo * STeo | | RSSSS OOOS |
| WV- | Geo * SGeoTeoTeoGeo * RA * ST * SC * ST * SG * RA * SA * | GGTTGATCTGAAGCAGCAGC | SOOOR SSSSR |
| 25472 | SG * SC * SA * SGeom5CeoAeoGeo * Sm5Ceo | | SSSSS OOOS |
| WV- | Geo * SGeoTeoTeoGeo * RA * ST * SC * ST * SG * SA * RA * | GGTTGATCTGAAGCAGCAGC | SOOOR SSSSS |
| 25473 | SG * SC * SA * SGeom5CeoAeoGeo * Sm5Ceo | | RSSSS OOOS |
| WV- | Geo * SGeoTeoTeoGeo * RA * ST * SC * ST * SG * SA * SA * | GGTTGATCTGAAGCAGCAGC | SOOOR SSSSS |
| 25474 | RG * SC * SA * SGeom5CeoAeoGeo * Sm5Ceo | | SRSSS OOOS |
| WV- | Geo * SGeoGeoTeoTeo * RG * SA * ST * SC * ST * SG * RA * | GGGTTGATCTGAAGCAGCAG | SOOOR SSSSS |
| 25475 | SA * SG * SC * SAeoGeom5CeoAeo * SGeo | | RSSSS OOOS |
| WV- | Geo * SGeoGeoTeoTeo * RG * SA * ST * SC * ST * SG * SA * | GGGTTGATCTGAAGCAGCAG | SOOOR SSSSS |
| 25476 | RA * SG * SC * SAeoGeom5CeoAeo * SGeo | | SRSSS OOOS |
| WV- | Geo * SGeoGeoTeoTeo * RG * SA * ST * SC * ST * SG * SA * | GGGTTGATCTGAAGCAGCAG | SOOOR SSSSS |
| 25477 | SA * RG * SC * SAeoGeom5CeoAeo * SGeo | | SSRSS OOOS |
| WV- | Geo * SGeoGeoGeoTeo * RT * SG * SA * ST * SC * ST * SG * | GGGGTTGATCTGAAGCAGCA | SOOOR SSSSS |
| 25478 | RA * SA * SG * Sm5CeoAeoGeom5Ceo * SAeo | | SRSSS OOOS |
| WV- | Geo * SGeoGeoGeoTeo * RT * SG * SA * ST * SC * ST * SG * | GGGGTTGATCTGAAGCAGCA | SOOOR SSSSS |
| 25479 | SA * RA * SG * Sm5CeoAeoGeom5Ceo * SAeo | | SSRSS OOOS |
| WV- | Geo * SGeoGeoGeoTeo * RT * SG * SA * ST * SC * ST * SG * | GGGGTTGATCTGAAGCAGCA | SOOOR SSSSS |
| 25480 | SA * SA * RG * Sm5CeoAeoGeom5Ceo * SAeo | | SSSRS OOOS |
| WV- | m5Ceo * SGeoGeoGeoGeo * RT * ST * SG * SA * ST * SC * ST * | CGGGGTTGATCTGAAGCAGC | SOOOR SSSSS |
| 25481 | SG * RA * SA * SGeom5CeoAeoGeom5Ceo | | SSRSS OOOO |
| WV- | m5Ceo * SGeoGeoGeoGeo * RT * ST * SG * SA * ST * SC * ST * | CGGGGTTGATCTGAAGCAGC | SOOOR SSSSS |
| 25482 | SG * SA * RA * SGeom5CeoAeoGeom5Ceo | | SSSRS OOOO |
| WV- | m5Ceo * SGeoGeoGeoGeo * RT * ST * SG * SA * ST * SC * ST * | CGGGGTTGATCTGAAGCAGC | SOOOR SSSSS |
| 25483 | SG * SA * SA * RGeom5CeoAeoGeom5Ceo | | SSSSR OOOO |
| WV- | Geo * SAeoTeom5CeoTeo * RG * RA * SA * SG * SC * SA * SG | GATCTGAAGCAGCAGCUUCU | SOOOR RSSSS |
| 25484 | * SC * SA * SG * SmC * SmU * SmU * SmC * SmU | | SSSSS SSSS |
| WV- | Geo * SAeoTeom5CeoTeo * RG * SA * RA * SG * SC * SA * SG | GATCTGAAGCAGCAGCUUCU | SOOOR SRSSS |
| 25485 | * SC * SA * SG * SmC * SmU * SmU * SmC * SmU | | SSSSS SSSS |
| WV- | Geo * SAeoTeom5CeoTeo * RG * SA * SA * RG * SC * SA * SG | GATCTGAAGCAGCAGCUUCU | SOOOR SSRSS |
| 25486 | * SC * SA * SG * SmC * SmU * SmU * SmC * SmU | | SSSSS SSSS |
| WV- | Teo * SGeoAeoTeom5Ceo * RT * SG * RA * SA * SG * SC * SA * | TGATCTGAAGCAGCAGCUUC | SOOOR SRSSS |
| 25487 | SG * SC * SA * SmG * SmC * SmU * SmU * SmC | | SSSSS SSSS |
| WV- | Teo * SGeoAeoTeom5Ceo * RT * SG * SA * RA * SG * SC * SA * | TGATCTGAAGCAGCAGCUUC | SOOOR SSRSS |
| 25488 | SG * SC * SA * SmG * SmC * SmU * SmU * SmC | | SSSSS SSSS |
| WV- | Teo * SGeoAeoTeom5Ceo * RT * SG * SA * SA * RG * SC * SA * | TGATCTGAAGCAGCAGCUUC | SOOOR SSSRS |
| 25489 | SG * SC * SA * SmG * SmC * SmU * SmU * SmC | | SSSSS SSSS |
| WV- | Teo * STeoGeoAeoTeo * RC * ST * SG * RA * SA * SG * SC * | TTGATCTGAAGCAGCAGCUU | SOOOR SSRSS |
| 25490 | SA * SG * SC * SmA * SmG * SmC * SmU * SmU | | SSSSS SSSS |
| WV- | Teo * STeoGeoAeoTeo * RC * ST * SG * SA * RA * SG * SC * | TTGATCTGAAGCAGCAGCUU | SOOOR SSSRS |
| 25491 | SA * SG * SC * SmA * SmG * SmC * SmU * SmU | | SSSSS SSSS |
| WV- | Teo * STeoGeoAeoTeo * RC * ST * SG * SA * SA * RG * SC * | TTGATCTGAAGCAGCAGCUU | SOOOR SSSSR |
| 25492 | SA * SG * SC * SmA * SmG * SmC * SmU * SmU | | SSSSS SSSS |
| WV- | Geo * STeoTeoGeoAeo * RT * SC * ST * SG * RA * SA * SG * | GTTGATCTGAAGCAGCAGCU | SOOOR SSSRS |
| 25493 | SC * SA * SG * SmC * SmA * SmG * SmC * SmU | | SSSSS SSSS |
| WV- | Geo * STeoTeoGeoAeo * RT * SC * ST * SG * SA * RA * SG * | GTTGATCTGAAGCAGCAGCU | SOOOR SSSSR |
| 25494 | SC * SA * SG * SmC * SmA * SmG * SmC * SmU | | SSSSS SSSS |
| WV- | Geo * STeoTeoGeoAeo * RT * SC * ST * SG * SA * SA * RG * | GTTGATCTGAAGCAGCAGCU | SOOOR SSSSS |
| 25495 | SC * SA * SG * SmC * SmA * SmG * SmC * SmU | | RSSSS SSSS |
| WV- | Geo * SGeoTeoTeoGeo * RA * ST * SC * ST * SG * RA * SA * | GGTTGATCTGAAGCAGCAGC | SOOOR SSSSR |
| 25496 | SG * SC * SA * SmG * SmC * SmA * SmG * SmC | | SSSSS SSSS |
| WV- | Geo * SGeoTeoTeoGeo * RA * ST * SC * ST * SG * SA * RA * | GGTTGATCTGAAGCAGCAGC | SOOOR SSSSS |
| 25497 | SG * SC * SA * SmG * SmC * SmA * SmG * SmC | | RSSSS SSSS |
| WV- | Geo * SGeoTeoTeoGeo * RA * ST * SC * ST * SG * SA * SA * | GGTTGATCTGAAGCAGCAGC | SOOOR SSSSS |
| 25498 | RG * SC * SA * SmG * SmC * SmA * SmG * SmC | | SRSSS SSSS |
| WV- | Geo * SGeoGeoTeoTeo * RG * SA * ST * SC * ST * SG * RA * | GGGTTGATCTGAAGCAGCAG | SOOOR SSSSS |
| 25499 | SA * SG * SC * SmA * SmG * SmC * SmA * SmG | | RSSSS SSSS |
| WV- | Geo * SGeoGeoTeoTeo * RG * SA * ST * SC * ST * SG * SA * | GGGTTGATCTGAAGCAGCAG | SOOOR SSSSS |
| 25500 | RA * SG * SC * SmA * SmG * SmC * SmA * SmG | | SRSSS SSSS |
| WV- | Geo * SGeoGeoTeoTeo * RG * SA * ST * SC * ST * SG * SA * | GGGTTGATCTGAAGCAGCAG | SOOOR SSSSS |
| 25501 | SA * RG * SC * SmA * SmG * SmC * SmA * SmG | | SSRSS SSSS |
| WV- | Geo * SGeoGeoGeoTeo * RT * SG * SA * ST * SC * ST * SG * | GGGGTTGATCTGAAGCAGCA | SOOOR SSSSS |
| 25502 | RA * SA * SG * SmC * SmA * SmG * SmC * SmA | | SRSSS SSSS |
| WV- | Geo * SGeoGeoGeoTeo * RT * SG * SA * ST * SC * ST * SG * | GGGGTTGATCTGAAGCAGCA | SOOOR SSSSS |
| 25503 | SA * RA * SG * SmC * SmA * SmG * SmC * SmA | | SSRSS SSSS |
| WV- | Geo * SGeoGeoGeoTeo * RT * SG * SA * ST * SC * ST * SG * | GGGGTTGATCTGAAGCAGCA | SOOOR SSSSS |
| 25504 | SA * SA * RG * SmC * SmA * SmG * SmC * SmA | | SSSRS SSSS |
| WV- | m5Ceo * SGeoGeoGeoGeo * RT * ST * SG * SA * ST * SC * ST * | CGGGGTTGATCTGAAGCAGC | SOOOR SSSSS |
| 25505 | SG * RA * SA * SmG * SmC * SmA * SmG * SmC | | SSRSS SSSS |
| WV- | m5Ceo * SGeoGeoGeoGeo * RT * ST * SG * SA * ST * SC * ST * | CGGGGTTGATCTGAAGCAGC | SOOOR SSSSS |
| 25506 | SG * SA * RA * SmG * SmC * SmA * SmG * SmC | | SSSRS SSSS |
| WV- | m5Ceo * SGeoGeoGeoGeo * RT * ST * SG * SA * ST * SC * ST * | CGGGGTTGATCTGAAGCAGC | SOOOR SSSSS |
| 25507 | SG * SA * SA * RmG * SmC * SmA * SmG * SmC | | SSSSR SSSS |
| WV- | mG * SmA * SmU * SmC * SmU * SG * RA * SA * SG * SC * SA | GAUCUGAAGCAGCAGCTTCT | SSSSS RSSSS |
| 25508 | * SG * SC * SA * SG * Sm5CeoTeoTeom5Ceo * STeo | | SSSSS OOOS |
| WV- | mG * SmA * SmU * SmC * SmU * SG * SA * RA * SG * SC * SA | GAUCUGAAGCAGCAGCTTCT | SSSSS SRSSS |
| 25509 | * SG * SC * SA * SG * Sm5CeoTeoTeom5Ceo * STeo | | SSSSS OOOS |
| WV- | mG * SmA * SmU * SmC * SmU * SG * SA * SA * RG * SC * SA | GAUCUGAAGCAGCAGCTTCT | SSSSS SSRSS |
| 25510 | * SG * SC * SA * SG * Sm5CeoTeoTeom5Ceo * STeo | | SSSSS OOOS |
| WV- | mU * SmG * SmA * SmU * SmC * ST * SG * RA * SA * SG * SC | UGAUCTGAAGCAGCAGCTTC | SSSSS SRSSS |
| 25511 | * SA * SG * SC * SA * SGeom5CeoTeoTeo* Sm5Ceo | | SSSSS OOOS |
| WV- | mU * SmG * SmA * SmU * SmC * ST * SG * SA * RA * SG * SC | UGAUCTGAAGCAGCAGCTTC | SSSSS SSRSS |
| 25512 | * SA * SG * SC * SA * SGeom5CeoTeoTeo * Sm5Ceo | | SSSSS OOOS |
| WV- | mU * SmG * SmA * SmU * SmC * ST * SG * SA * SA * RG * SC | UGAUCTGAAGCAGCAGCTTC | SSSSS SSSRS |
| 25513 | * SA * SG * SC * SA * SGeom5CeoTeoTeo * Sm5Ceo | | SSSSS OOOS |
| WV- | mU * SmU * SmG * SmA * SmU * SC * ST * SG * RA * SA * SG | UUGAUCTGAAGCAGCAGCTT | SSSSS SSRSS |
| 25514 | * SC * SA * SG * SC * SAeoGeom5CeoTeo * STeo | | SSSSS OOOS |
| WV- | mU * SmU * SmG * SmA * SmU * SC * ST * SG * SA * RA * SG | UUGAUCTGAAGCAGCAGCTT | SSSSS SSSRS |
| 25515 | * SC * SA * SG * SC * SAeoGeom5CeoTeo * STeo | | SSSSS OOOS |
| WV- | mU * SmU * SmG * SmA * SmU * SC * ST * SG * SA * SA * RG | UUGAUCTGAAGCAGCAGCTT | SSSSS SSSSR |
| 25516 | * SC * SA * SG * SC * SAeoGeom5CeoTeo * STeo | | SSSSS OOOS |
| WV- | mG * SmU * SmU * SmG * SmA * ST * SC * ST * SG * RA * SA | GUUGATCTGAAGCAGCAGCT | SSSSS SSSRS |
| 25517 | * SG * SC * SA * SG * Sm5CeoAeoGeom5Ceo * STeo | | SSSSS OOOS |
| WV- | mG * SmU * SmU * SmG * SmA * ST * SC * ST * SG * SA * RA | GUUGATCTGAAGCAGCAGCT | SSSSS SSSSR |
| 25518 | * SG * SC * SA * SG * Sm5CeoAeoGeom5Ceo * STeo | | SSSSS OOOS |
| WV- | mG * SmU * SmU * SmG * SmA * ST * SC * ST * SG * SA * SA | GUUGATCTGAAGCAGCAGCT | SSSSS SSSSS |
| 25519 | * RG * SC * SA * SG * Sm5CeoAeoGeom5Ceo * STeo | | RSSSS OOOS |
| WV- | mG * SmG * SmU * SmU * SmG * SA * ST * SC * ST * SG * RA | GGUUGATCTGAAGCAGCAGC | SSSSS SSSSR |
| 25520 | * SA * SG * SC * SA * SGeom5CeoAeoGeo * Sm5Ceo | | SSSSS OOOS |
| WV- | mG * SmG * SmU * SmU * SmG * SA * ST * SC * ST * SG * SA | GGUUGATCTGAAGCAGCAGC | SSSSS SSSSS |
| 25521 | * RA * SG * SC * SA * SGeom5CeoAeoGeo * Sm5Ceo | | RSSSS OOOS |
| WV- | mG * SmG * SmU * SmU * SmG * SA * ST * SC * ST * SG * SA | GGUUGATCTGAAGCAGCAGC | SSSSS SSSSS |
| 25522 | * SA * RG * SC * SA * SGeom5CeoAeoGeo * Sm5Ceo | | SRSSS OOOS |
| WV- | mG * SmG * SmG * SmU * SmU * SG * SA * ST * SC * ST * SG | GGGUUGATCTGAAGCAGCAG | SSSSS SSSSS |
| 25523 | * RA * SA * SG * SC * SAeoGeom5CeoAeo * SGeo | | RSSSS OOOS |
| WV- | mG * SmG * SmG * SmU * SmU * SG * SA * ST * SC * ST * SG | GGGUUGATCTGAAGCAGCAG | SSSSS SSSSS |
| 25524 | * SA * RA * SG * SC * SAeoGeom5CeoAeo * SGeo | | SRSSS OOOS |
| WV- | mG * SmG * SmG * SmU * SmU * SG * SA * ST * SC * ST * SG | GGGUUGATCTGAAGCAGCAG | SSSSS SSSSS |
| 25525 | * SA * SA * RG * SC * SAeoGeom5CeoAeo * SGeo | | SSRSS OOOS |
| WV- | mG * SmG * SmG * SmG * SmU * ST * SG * SA * ST * SC * ST | GGGGUTGATCTGAAGCAGCA | SSSSS SSSSS |
| 25526 | * SG * RA * SA * SG * 5m5CeoAeoGeom5Ceo * SAeo | | SRSSS OOOS |
| WV- | mG * SmG * SmG * SmG * SmU * ST * SG * SA * ST * SC * ST | GGGGUTGATCTGAAGCAGCA | SSSSS SSSSS |
| 25527 | * SG * SA * RA * SG * Sm5CeoAeoGeom5Ceo * SAeo | | SSRSS OOOS |
| WV- | mG * SmG * SmG * SmG * SmU * ST * SG * SA * ST * SC * ST | GGGGUTGATCTGAAGCAGCA | SSSSS SSSSS |
| 25528 | * SG * SA * SA * RG * Sm5CeoAeoGeom5Ceo * SAeo | | SSSRS OOOS |
| WV- | m5mC * SmG * SmG * SmG * SmG * ST * ST * SG * SA * ST * | CGGGGTTGATCTGAAGCAGC | SSSSS SSSSS |
| 25529 | SC * ST * SG * RA * SA * SGeom5CeoAeoGeom5Ceo | | SSRSS OOOO |
| WV- | m5mC * SmG * SmG * SmG * SmG * ST * ST * SG * SA * ST * | CGGGGTTGATCTGAAGCAGC | SSSSS SSSSS |
| 25530 | SC * ST * SG * SA * RA * SGeom5CeoAeoGeom5Ceo | | SSSRS OOOO |
| WV- | m5mC * SmG * SmG * SmG * SmG * ST * ST * SG * SA * ST * | CGGGGTTGATCTGAAGCAGC | SSSSS SSSSS |
| 25531 | SC * ST * SG * SA * SA * RGeom5CeoAeoGeom5Ceo | | SSSSR OOOO |
| WV- | Geo * SAeoTeom5CeoTeo * RG * RT * SA * SG * SC * SA * SG * | GATCTGTAGCAGCAGCTTCT | SOOOR RSSSS |
| 27829 | SC * SA * SG * Sm5CeoTeoTeom5Ceo * STeo | | SSSSS OOOS |
| WV- | Geo * SAeoTeom5CeoTeo * RG * ST * RA * SG * SC * SA * SG * | GATCTGTAGCAGCAGCTTCT | SOOOR SRSSS |
| 27830 | SC * SA * SG * Sm5CeoTeoTeom5Ceo * STeo | | SSSSS OOOS |
| WV- | Geo * SAeoTeom5CeoTeo * RG * ST * SA * RG * SC * SA * SG * | GATCTGTAGCAGCAGCTTCT | SOOOR SSRSS |
| 27831 | SC * SA * SG * Sm5CeoTeoTeom5Ceo * STeo | | SSSSS OOOS |
| WV- | Teo * SGeoAeoTeom5Ceo * RT * SG * RT * SA * SG * SC * SA * | TGATCTGTAGCAGCAGCTTC | SOOOR SRSSS |
| 27832 | SG * SC * SA * SGeom5CeoTeoTeo * Sm5Ceo | | SSSSS OOOS |
| WV- | Teo * SGeoAeoTeom5Ceo * RT * SG * ST * RA * SG * SC * SA * | TGATCTGTAGCAGCAGCTTC | SOOOR SSRSS |
| 27833 | SG * SC * SA * SGeom5CeoTeoTeo * Sm5Ceo | | SSSSS OOOS |
| WV- | Teo * SGeoAeoTeom5Ceo * RT * SG * ST * SA * RG * SC * SA * | TGATCTGTAGCAGCAGCTTC | SOOOR SSSRS |
| 27834 | SG * SC * SA * SGeom5CeoTeoTeo * Sm5Ceo | | SSSSS OOOS |
| WV- | Teo * STeoGeoAeoTeo * RC * ST * SG * RT * SA * SG * SC * SA | TTGATCTGTAGCAGCAGCTT | SOOOR SSRSS |
| 27835 | * SG * SC * SAeoGeom5CeoTeo * STeo | | SSSSS OOOS |
| WV- | Teo * STeoGeoAeoTeo * RC * ST * SG * ST * RA * SG * SC * SA | TTGATCTGTAGCAGCAGCTT | SOOOR SSSRS |
| 27836 | * SG * SC * SAeoGeom5CeoTeo * STeo | | SSSSS OOOS |
| WV- | Teo * STeoGeoAeoTeo * RC * ST * SG * ST * SA * RG * SC * SA | TTGATCTGTAGCAGCAGCTT | SOOOR SSSSR |
| 27837 | * SG * SC * SAeoGeom5CeoTeo * STeo | | SSSSS OOOS |
| WV- | Geo * STeoTeoGeoAeo * RT * SC * ST * SG * RT * SA * SG * SC | GTTGATCTGTAGCAGCAGCT | SOOOR SSSRS |
| 27838 | * SA * SG * Sm5CeoAeoGeom5Ceo * STeo | | SSSSS OOOS |
| WV- | Geo * STeoTeoGeoAeo * RT * SC * ST * SG * ST * RA * SG * SC | GTTGATCTGTAGCAGCAGCT | SOOOR SSSSR |
| 27839 | * SA * SG * Sm5CeoAeoGeom5Ceo * STeo | | SSSSS OOOS |
| WV- | Geo * SGeoTeoTeoGeo * RA * ST * SC * ST * SG * RT * SA * | GGTTGATCTGTAGCAGCAGC | SOOOR SSSSR |
| 27840 | SG * SC * SA * SGeom5CeoAeoGeo * Sm5Ceo | | SSSSS OOOS |
| WV- | Geo * SGeoTeoTeoGeo * RA * ST * SC * ST * SG * ST * RA * | GGTTGATCTGTAGCAGCAGC | SOOOR SSSSS |
| 27841 | SG * SC * SA * SGeom5CeoAeoGeo * Sm5Ceo | | RSSSS OOOS |
| WV- | Geo * SGeoTeoTeoGeo * RA * ST * SC * ST * SG * ST * SA * | GGTTGATCTGTAGCAGCAGC | SOOOR SSSSS |
| 27842 | RG * SC * SA * SGeom5CeoAeoGeo * Sm5Ceo | | SRSSS OOOS |
| WV- | Geo * SGeoGeoTeoTeo * RG * SA * ST * SC * ST * SG * RT * | GGGTTGATCTGTAGCAGCAG | SOOOR SSSSS |
| 27843 | SA * SG * SC * SAeoGeom5CeoAeo * SGeo | | RSSSS OOOS |
| WV- | Geo * SGeoGeoTeoTeo * RG * SA * ST * SC * ST * SG * ST * | GGGTTGATCTGTAGCAGCAG | SOOOR SSSSS |
| 27844 | RA * SG * SC * SAeoGeom5CeoAeo * SGeo | | SRSSS OOOS |
| WV- | Geo * SGeoGeoTeoTeo * RG * SA * ST * SC * ST * SG * ST * SA | GGGTTGATCTGTAGCAGCAG | SOOOR SSSSS |
| 27845 | * RG * SC * SAeoGeom5CeoAeo * SGeo | | SSRSS OOOS |
| WV- | Geo * SGeoGeoGeoTeo * RT * SG * SA * ST * SC * ST * SG * | GGGGTTGATCTGTAGCAGCA | SOOOR SSSSS |
| 27846 | RT * SA * SG * Sm5CeoAeoGeom5Ceo * SAeo | | SRSSS OOOS |
| WV- | Geo * SGeoGeoGeoTeo * RT * SG * SA * ST * SC * ST * SG * ST | GGGGTTGATCTGTAGCAGCA | SOOOR SSSSS |
| 27847 | * RA * SG * Sm5CeoAeoGeom5Ceo * SAeo | | SSRSS OOOS |
| WV- | Geo * SGeoGeoGeoTeo * RT * SG * SA * ST * SC * ST * SG * ST | GGGGTTGATCTGTAGCAGCA | SOOOR SSSSS |
| 27848 | * SA * RG * Sm5CeoAeoGeom5Ceo * SAeo | | SSSRS OOOS |
| WV- | m5Ceo * SGeoGeoGeoGeo * RT * ST * SG * SA * ST * SC * ST * | CGGGGTTGATCTGTAGCAGC | SOOOR SSSSS |
| 27849 | SG * RT * SA * SGeom5CeoAeoGeom5Ceo | | SSRSS OOOO |
| WV- | m5Ceo * SGeoGeoGeoGeo * RT * ST * SG * SA * ST * SC * ST * | CGGGGTTGATCTGTAGCAGC | SOOOR SSSSS |
| 27850 | SG * ST * RA * SGeom5CeoAeoGeom5Ceo | | SSSRS OOOO |
| WV- | m5Ceo * SGeoGeoGeoGeo * RT * ST * SG * SA * ST * SC * ST * | CGGGGTTGATCTGTAGCAGC | SOOOR SSSSS |
| 27851 | SG * ST * SA * RGeom5CeoAeoGeom5Ceo | | SSSSR OOOO |
| WV- | Geo * SAeoTeom5CeoTeo * RG * RT * SA * SG * SC * SA * SG * | GATCTGTAGCAGCAGCUUCU | SOOOR RSSSS |
| 27852 | SC * SA * SG * SmC * SmU * SmU * SmC * SmU | | SSSSS SSSS |
| WV- | Geo * SAeoTeom5CeoTeo * RG * ST * RA * SG * SC * SA * SG * | GATCTGTAGCAGCAGCUUCU | SOOOR SRSSS |
| 27853 | SC * SA * SG * SmC * SmU * SmU * SmC * SmU | | SSSSS SSSS |
| WV- | Geo * SAeoTeom5CeoTeo * RG * ST * SA * RG * SC * SA * SG * | GATCTGTAGCAGCAGCUUCU | SOOOR SSRSS |
| 27854 | SC * SA * SG * SmC * SmU * SmU * SmC * SmU | | SSSSS SSSS |
| WV- | Teo * SGeoAeoTeom5Ceo * RT * SG * RT * SA * SG * SC * SA * | TGATCTGTAGCAGCAGCUUC | SOOOR SRSSS |
| 27855 | SG * SC * SA * SmG * SmC * SmU * SmU * SmC | | SSSSS SSSS |
| WV- | Teo * SGeoAeoTeom5Ceo * RT * SG * ST * RA * SG * SC * SA * | TGATCTGTAGCAGCAGCUUC | SOOOR SSRSS |
| 27856 | SG * SC * SA * SmG * SmC * SmU * SmU * SmC | | SSSSS SSSS |
| WV- | Teo * SGeoAeoTeom5Ceo * RT * SG * ST * SA * RG * SC * SA * | TGATCTGTAGCAGCAGCUUC | SOOOR SSSRS |
| 27857 | SG * SC * SA * SmG * SmC * SmU * SmU * SmC | | SSSSS SSSS |
| WV- | Teo * STeoGeoAeoTeo * RC * ST * SG * RT * SA * SG * SC * SA | TTGATCTGTAGCAGCAGCUU | SOOOR SSRSS |
| 27858 | * SG * SC * SmA * SmG * SmC * SmU * SmU | | SSSSS SSSS |
| WV- | Teo * STeoGeoAeoTeo * RC * ST * SG * ST * RA * SG * SC * SA | TTGATCTGTAGCAGCAGCUU | SOOOR SSSRS |
| 27859 | * SG * SC * SmA * SmG * SmC * SmU * SmU | | SSSSS SSSS |
| WV- | Teo * STeoGeoAeoTeo * RC * ST * SG * ST * SA * RG * SC * SA | TTGATCTGTAGCAGCAGCUU | SOOOR SSSSR |
| 27860 | * SG * SC * SmA * SmG * SmC * SmU * SmU | | SSSSS SSSS |
| WV- | Geo * STeoTeoGeoAeo * RT * SC * ST * SG * RT * SA * SG * SC | GTTGATCTGTAGCAGCAGCU | SOOOR SSSRS |
| 27861 | * SA * SG * SmC * SmA * SmG * SmC * SmU | | SSSSS SSSS |
| WV- | Geo * STeoTeoGeoAeo * RT * SC * ST * SG * ST * RA * SG * SC | GTTGATCTGTAGCAGCAGCU | SOOOR SSSSR |
| 27862 | * SA * SG * SmC * SmA * SmG * SmC * SmU | | SSSSS SSSS |
| WV- | Geo * STeoTeoGeoAeo * RT * SC * ST * SG * ST * SA * RG * SC | GTTGATCTGTAGCAGCAGCU | SOOOR SSSSS |
| 27863 | * SA * SG * SmC * SmA * SmG * SmC * SmU | | RSSSS SSSS |
| WV- | Geo * SGeoTeoTeoGeo * RA * ST * SC * ST * SG * RT * SA * | GGTTGATCTGTAGCAGCAGC | SOOOR SSSSR |
| 27864 | SG * SC * SA * SmG * SmC * SmA * SmG * SmC | | SSSSS SSSS |
| WV- | Geo * SGeoTeoTeoGeo * RA * ST * SC * ST * SG * ST * RA * | GGTTGATCTGTAGCAGCAGC | SOOOR SSSSS |
| 27865 | SG * SC * SA * SmG * SmC * SmA * SmG * SmC | | RSSSS SSSS |
| WV- | Geo * SGeoTeoTeoGeo * RA * ST * SC * ST * SG * ST * SA * | GGTTGATCTGTAGCAGCAGC | SOOOR SSSSS |
| 27866 | RG * SC * SA * SmG * SmC * SmA * SmG * SmC | | SRSSS SSSS |
| WV- | Geo * SGeoGeoTeoTeo * RG * SA * ST * SC * ST * SG * RT * | GGGTTGATCTGTAGCAGCAG | SOOOR SSSSS |
| 27867 | SA * SG * SC * SmA * SmG * SmC * SmA * SmG | | RSSSS SSSS |
| WV- | Geo * SGeoGeoTeoTeo * RG * SA * ST * SC * ST * SG * ST * | GGGTTGATCTGTAGCAGCAG | SOOOR SSSSS |
| 27868 | RA * SG * SC * SmA * SmG * SmC * SmA * SmG | | SRSSS SSSS |
| WV- | Geo * SGeoGeoTeoTeo * RG * SA * ST * SC * ST * SG * ST * SA | GGGTTGATCTGTAGCAGCAG | SOOOR SSSSS |
| 27869 | * RG * SC * SmA * SmG * SmC * SmA * SmG | | SSRSS SSSS |
| WV- | Geo * SGeoGeoGeoTeo * RT * SG * SA * ST * SC * ST * SG * | GGGGTTGATCTGTAGCAGCA | SOOOR SSSSS |
| 27870 | RT * SA * SG * SmC * SmA * SmG * SmC * SmA | | SRSSS SSSS |
| WV- | Geo * SGeoGeoGeoTeo * RT * SG * SA * ST * SC * ST * SG * ST | GGGGTTGATCTGTAGCAGCA | SOOOR SSSSS |
| 27871 | * RA * SG * SmC * SmA * SmG * SmC * SmA | | SSRSS SSSS |
| WV- | Geo * SGeoGeoGeoTeo * RT * SG * SA * ST * SC * ST * SG * ST | GGGGTTGATCTGTAGCAGCA | SOOOR SSSSS |
| 27872 | * SA * RG * SmC * SmA * SmG * SmC * SmA | | SSSRS SSSS |
| WV- | m5Ceo * SGeoGeoGeoGeo * RT * ST * SG * SA * ST * SC * ST * | CGGGGTTGATCTGTAGCAGC | SOOOR SSSSS |
| 27873 | SG * RT * SA * SmG * SmC * SmA * SmG * SmC | | SSRSS SSSS |
| WV- | m5Ceo * SGeoGeoGeoGeo * RT * ST * SG * SA * ST * SC * ST * | CGGGGTTGATCTGTAGCAGC | SOOOR SSSSS |
| 27874 | SG * ST * RA * SmG * SmC * SmA * SmG * SmC | | SSSRS SSSS |
| WV- | m5Ceo * SGeoGeoGeoGeo * RT * ST * SG * SA * ST * SC * ST * | CGGGGTTGATCTGTAGCAGC | SOOOR SSSSS |
| 27875 | SG * ST * SA * RmG * SmC * SmA * SmG * SmC | | SSSSR SSSS |
| WV- | mG * SmA * SmU * SmC * SmU * SG * RT * SA * SG * SC * SA | GAUCUGTAGCAGCAGCTTCT | SSSSS RSSSS |
| 27876 | * SG * SC * SA * SG * Sm5CeoTeoTeom5Ceo * STeo | | SSSSS OOOS |
| WV- | mG * SmA * SmU * SmC * SmU * SG * ST * RA * SG * SC * SA | GAUCUGTAGCAGCAGCTTCT | SSSSS SRSSS |
| 27877 | * SG * SC * SA * SG * Sm5CeoTeoTeom5Ceo * STeo | | SSSSS OOOS |
| WV- | mG * SmA * SmU * SmC * SmU * SG * ST * SA * RG * SC * SA | GAUCUGTAGCAGCAGCTTCT | SSSSS SSRSS |
| 27878 | * SG * SC * SA * SG * Sm5CeoTeoTeom5Ceo * STeo | | SSSSS OOOS |
| WV- | mU * SmG * SmA * SmU * SmC * ST * SG * RT * SA * SG * SC | UGAUCTGTAGCAGCAGCTTC | SSSSS SRSSS |
| 27879 | * SA * SG * SC * SA * SGeom5CeoTeoTeo 5m5Ceo | | SSSSS OOOS |
| WV- | mU * SmG * SmA * SmU * SmC * ST * SG * ST * RA * SG * SC | UGAUCTGTAGCAGCAGCTTC | SSSSS SSRSS |
| 27880 | * SA * SG * SC * SA * SGeom5CeoTeoTeo * 5m5Ceo | | SSSSS OOOS |
| WV- | mU * SmG * SmA * SmU * SmC * ST * SG * ST * SA * RG * SC | UGAUCTGTAGCAGCAGCTTC | SSSSS SSSRS |
| 27881 | * SA * SG * SC * SA * SGeom5CeoTeoTeo * 5m5Ceo | | SSSSS OOOS |
| WV- | mU * SmU * SmG * SmA * SmU * SC * ST * SG * RT * SA * SG | UUGAUCTGTAGCAGCAGCTT | SSSSS SSRSS |
| 27882 | * SC * SA * SG * SC * SAeoGeom5CeoTeo * STeo | | SSSSS OOOS |
| WV- | mU * SmU * SmG * SmA * SmU * SC * ST * SG * ST * RA * SG | UUGAUCTGTAGCAGCAGCTT | SSSSS SSSRS |
| 27883 | * SC * SA * SG * SC * SAeoGeom5CeoTeo * STeo | | SSSSS OOOS |
| WV- | mU * SmU * SmG * SmA * SmU * SC * ST * SG * ST * SA * RG | UUGAUCTGTAGCAGCAGCTT | SSSSS SSSSR |
| 27884 | * SC * SA * SG * SC * SAeoGeom5CeoTeo * STeo | | SSSSS OOOS |
| WV- | mG * SmG * SmU * SmU * SmG * SA * ST * SC * ST * SG * RT | GGUUGATCTGTAGCAGCAGC | SSSSS SSSSR |
| 27885 | * SA * SG * SC * SA * SGeom5CeoAeoGeo * Sm5Ceo | | SSSSS OOOS |
| WV- | mG * SmG * SmU * SmU * SmG * SA * ST * SC * ST * SG * ST | GGUUGATCTGTAGCAGCAGC | SSSSS SSSSS |
| 27886 | * RA * SG * SC * SA * SGeom5CeoAeoGeo * Sm5Ceo | | RSSSS OOOS |
| WV- | mG * SmG * SmU * SmU * SmG * SA * ST * SC * ST * SG * ST | GGUUGATCTGTAGCAGCAGC | SSSSS SSSSS |
| 27887 | * SA * RG * SC * SA * SGeom5CeoAeoGeo * Sm5Ceo | | SRSSS OOOS |
| WV- | mG * SmG * SmG * SmU * SmU * SG * SA * ST * SC * ST * SG | GGGUUGATCTGTAGCAGCAG | SSSSS SSSSS |
| 27888 | * RT * SA * SG * SC * SAeoGeom5CeoAeo * SGeo | | RSSSS OOOS |
| WV- | mG * SmG * SmG * SmU * SmU * SG * SA * ST * SC * ST * SG | GGGUUGATCTGTAGCAGCAG | SSSSS SSSSS |
| 27889 | * ST * RA * SG * SC * SAeoGeom5CeoAeo * SGeo | | SRSSS OOOS |
| WV- | mG * SmG * SmG * SmU * SmU * SG * SA * ST * SC * ST * SG | GGGUUGATCTGTAGCAGCAG | SSSSS SSSSS |
| 27890 | * ST * SA * RG * SC * SAeoGeom5CeoAeo * SGeo | | SSRSS OOOS |
| WV- | mG * SmG * SmG * SmG * SmU * ST * SG * SA * ST * SC * ST | GGGGUTGATCTGTAGCAGCA | SSSSS SSSSS |
| 27891 | * SG * RT * SA * SG * Sm5CeoAeoGeom5Ceo * SAeo | | SRSSS OOOS |
| WV- | mG * SmG * SmG * SmG * SmU * ST * SG * SA * ST * SC * ST | GGGGUTGATCTGTAGCAGCA | SSSSS SSSSS |
| 27892 | * SG * ST * RA * SG * Sm5CeoAeoGeom5Ceo * SAeo | | SSRSS OOOS |
| WV- | mG * SmG * SmG * SmG * SmU * ST * SG * SA * ST * SC * ST | GGGGUTGATCTGTAGCAGCA | SSSSS SSSSS |
| 27893 | * SG * ST * SA * RG * Sm5CeoAeoGeom5Ceo * SAeo | | SSSRS OOOS |
| WV- | m5mC * SmG * SmG * SmG * SmG * ST * ST * SG * SA * ST * | CGGGGTTGATCTGTAGCAGC | SSSSS SSSSS |
| 27894 | SC * ST * SG * RT * SA * SGeom5CeoAeoGeom5Ceo | | SSRSS OOOO |
| WV- | m5mC * SmG * SmG * SmG * SmG * ST * ST * SG * SA * ST * | CGGGGTTGATCTGTAGCAGC | SSSSS SSSSS |
| 27895 | SC * ST * SG * ST * RA * SGeom5CeoAeoGeom5Ceo | | SSSRS OOOO |
| WV- | m5mC * SmG * SmG * SmG * SmG * ST * ST * SG * SA * ST * | CGGGGTTGATCTGTAGCAGC | SSSSS SSSSS |
| 27896 | SC * ST * SG * ST * SA * RGeom5CeoAeoGeom5Ceo | | SSSSR OOOO |
| WV- | m5Ceo * SGeoGeoGeoGeo * RT * ST * SG * SA * ST * SC * ST * | CGGGGTTGATCTGTAGCAGC | SOOOR SSSSS |
| 27905 | SG * RT * SA * SGeom5CeoAeoGeo * Sm5Ceo | | SSRSS OOOS |
| WV- | m5Ceo * SGeoGeoGeoGeo * RT * ST * SG * SA * ST * SC * ST * | CGGGGTTGATCTGTAGCAGC | SOOOR SSSSS |
| 27906 | SG * ST * RA * SGeom5CeoAeoGeo * Sm5Ceo | | SSSRS OOOS |
| WV- | m5Ceo * SGeoGeoGeoGeo * RT * ST * SG * SA * ST * SC * ST * | CGGGGTTGATCTGTAGCAGC | SOOOR SSSSS |
| 27907 | SG * ST * SA * RGeom5CeoAeoGeo * Sm5Ceo | | SSSSR OOOS |
| WV- | m5mC * SmG * SmG * SmG * SmG * ST * ST * SG * SA * ST * | CGGGGTTGATCTGTAGCAGC | SSSSS SSSSS |
| 27908 | SC * ST * SG * RT * SA * SGeom5CeoAeoGeo * Sm5Ceo | | SSRSS OOOS |
| WV- | m5mC * SmG * SmG * SmG * SmG * ST * ST * SG * SA * ST * | CGGGGTTGATCTGTAGCAGC | SSSSS SSSSS |
| 27909 | SC * ST * SG * ST * RA * SGeom5CeoAeoGeo * Sm5Ceo | | SSSRS OOOS |
| WV- | m5mC * SmG * SmG * SmG * SmG * ST * ST * SG * SA * ST * | CGGGGTTGATCTGTAGCAGC | SSSSS SSSSS |
| 27910 | SC * ST * SG * ST * SA * RGeom5CeoAeoGeo * Sm5Ceo | | SSSSR OOOS |
| WV- | Mod001L001mA * Sm5Ceom5CeoGeomC * SG * SA * SC * RC * | ACCGCGACCCTCTGGACAGG | OSOOO SSSRS |
| 27969 | SC * ST * SC * ST * SG * SG * RmA * SmC * SmA * SmG * SmG | | SSSSS RSSSS |
| WV- | L001mA * Sm5Ceom5CeoGeomC * SG * SA * SC * RC * SC * ST | ACCGCGACCCTCTGGACAGG | OSOOO SSSRS |
| 27970 | * SC * ST * SG * SG * RmA * SmC * SmA * SmG * SmG | | SSSSS RSSSS |
| WV- | mA * m5Ceom5CeoGeomC * C * A * T * C * C * C * m5C * G * | ACCGCCATCCCCGCCGUAGC | XOOOX XXXXX |
| 28151 | C * m5C * mG * mU * mA * mG * mC | | XXXXX XXXX |
| WV- | mA * Sm5Ceom5CeoGeomC * SC * SA * ST * SC * RC * SC * | ACCGCCATCCCCGCCGUAGC | SOOOS SSSRS |
| 28152 | Sm5C * SG * RC * Sm5C * SmG * SmU * SmA * SmG * SmC | | SSRSS SSSS |
| WV- | mA * Sm5Ceom5CeoGeomC * SC * SA * ST * SC * SC * RC * | ACCGCCATCCCCGCCGUAGC | SOOOS SSSSR |
| 28153 | Sm5C * SG * RC * Sm5C * SmG * SmU * SmA * SmG * SmC | | SSRSS SSSS |
| WV- | mA * Sm5Ceom5CeoGeomC * SC * SA * ST * RC * SC * SC * | ACCGCCATCCCCGCCGUAGC | SOOOS SSRSS |
| 28154 | Rm5C * SG * SC * Sm5C * SmG * SmU * SmA * SmG * SmC | | RSSSS SSSS |
| WV- | mA * Sm5Ceom5CeoGeomC * SC * SA * RT * SC * SC * RC * | ACCGCCATCCCCGCCGUAGC | SOOOS SRSSR |
| 28155 | Sm5C * SG * RC * Sm5C * SmG * SmU * SmA * SmG * SmC | | SSRSS SSSS |
| WV- | mG *TeoTeoAeomC *m5C * G * C * C * A * T * C * C * C * | GTTACCGCCATCCCCGCCGU | XOOOX XXXXX |
| 28156 | m5C * mG * mC * m5mC * mG * mU | | XXXXX XXXX |
| WV- | mG * STeoTeoAeomC * Sm5C * SG * SC * SC * RA * ST * SC * | GTTACCGCCATCCCCGCCGU | SOOOS SSSRS |
| 28157 | SC * RC * Sm5C * SmG * SmC * Sm5mC * SmG * SmU | | SSRSS SSSS |
| WV- | mG * STeoTeoAeomC * Sm5C * SG * SC * SC * RA * ST * SC * | GTTACCGCCATCCCCGCCGU | SOOOS SSSRS |
| 28158 | RC * SC * Sm5C * SmG * SmC * Sm5mC * SmG * SmU | | SRSSS SSSS |
| WV- | mG * STeoTeoAeomC * Sm5C * SG * SC * SC * SA * RT * SC * | GTTACCGCCATCCCCGCCGU | SOOOS SSSSR |
| 28159 | SC * RC * Sm5C * SmG * SmC * Sm5mC * SmG * SmU | | SSRSS SSSS |
| WV- | mG * STeoTeoAeomC * Sm5C * SG * RC * SC * SA * RT * SC * | GTTACCGCCATCCCCGCCGU | SOOOS SRSSR |
| 28160 | SC * RC * Sm5C * SmG * SmC * Sm5mC * SmG * SmU | | SSRSS SSSS |
| WV- | mG * SGeom5CeoTeomC * ST * SG * RG * SG * ST * ST * SG * | GGCTCTGGGTTGCTGGGUCA | SOOOS SRSSS |
| 28161 | SC * RT * SG * SmG * SmG * SmU * SmC * SmA | | SSRSS SSSS |
| WV- | mG * SGeom5CeoTeomC * ST * SG * RG * SG * ST * ST * SG * | GGCTCTGGGTTGCTGGGUCA | SOOOS SRSSS |
| 28162 | RC * ST * SG * SmG * SmG * SmU * SmC * SmA | | SRSSS SSSS |
| WV- | mG * SGeom5CeoTeomC * ST * SG * RG * SG * ST * ST * RG * | GGCTCTGGGTTGCTGGGUCA | SOOOS SRSSS |
| 28163 | SC * ST * SG * SmG * SmG * SmU * SmC * SmA | | RSSSS SSSS |
| WV- | mG * SGeom5CeoTeomC * ST * SG * RG * SG * ST * RT * SG * | GGCTCTGGGTTGCTGGGUCA | SOOOS SRSSR |
| 28164 | SC * RT * SG * SmG * SmG * SmU * SmC * SmA | | SSRSS SSSS |
| WV- | mG * SGeoTeoGeomU * SC * SC * RC * ST * SC * SA * ST * SG | GGTGUCCCTCATGGGCUCUG | SOOOS SRSSS |
| 28165 | * RG * SG * SmC * SmU * SmC * SmU * SmG | | SSRSS SSSS |
| WV- | mG * SGeoTeoGeomU * SC * SC * RC * ST * SC * RA * ST * SG | GGTGUCCCTCATGGGCUCUG | SOOOS SRSSR |
| 28166 | * SG * SG * SmC * SmU * SmC * SmU * SmG | | SSSSS SSSS |
| WV- | mG * SGeoTeoGeomU * SC * SC * SC * RT * SC * SA * ST * SG | GGTGUCCCTCATGGGCUCUG | SOOOS SSRSS |
| 28167 | * RG * SG * SmC * SmU * SmC * SmU * SmG | | SSRSS SSSS |
| WV- | mG * SGeoTeoGeomU * SC * SC * RC * ST * SC * RA * ST * SG | GGTGUCCCTCATGGGCUCUG | SOOOS SRSSR |
| 28168 | * RG * SG * SmC * SmU * SmC * SmU * SmG | | SSRSS SSSS |
| WV- | mA * Sm5Ceom5CeoGeomC * SG * SA * RC * SC * SC * ST * SC | ACCGCGACCCTCTGGACAGG | SOOOS SRSSS |
| 28802 | * ST * SG * SG * SmA * SmC * SmA * SmG * SmG | | SSSSS SSSS |
| WV- | mA * Sm5Ceom5CeoGeomC * SG * SA * SC * RC * SC * ST * SC | ACCGCGACCCTCTGGACAGG | SOOOS SSRSS |
| 28803 | * ST * SG * SG * SmA * SmC * SmA * SmG * SmG | | SSSSS SSSS |
| WV- | mA * Sm5Ceom5CeoGeomC * SG * SA * SC * SC * RC * ST * SC | ACCGCGACCCTCTGGACAGG | SOOOS SSSRS |
| 28804 | * ST * SG * SG * SmA * SmC * SmA * SmG * SmG | | SSSSS SSSS |
| WV- | mA * Sm5Ceom5CeoGeomC * SG * SA * SC * SC * SC * RT * SC | ACCGCGACCCTCTGGACAGG | SOOOS SSSSR |
| 28805 | * ST * SG * SG * SmA * SmC * SmA * SmG * SmG | | SSSSS SSSS |
| WV- | mA * Sm5Ceom5CeoGeomC * SG * SA * SC * SC * SC * ST * SC | ACCGCGACCCTCTGGACAGG | SOOOS SSSSS |
| 28806 | * ST * RG * SG * SmA * SmC * SmA * SmG * SmG | | SSRSS SSSS |
| WV- | mA * Sm5Ceom5CeoGeomC * SG * SA * RC * SC * SC * ST * SC | ACCGCGACCCTCTGGACAGG | SOOOS SRSSS |
| 28807 | * ST * SG * SGmA * SmC * SmA * SmG * SmG | | SSSSO SSSS |
| WV- | mA * Sm5Ceom5CeoGeomC * SG * SA * SC * RC * SC * ST * SC | ACCGCGACCCTCTGGACAGG | SOOOS SSRSS |
| 28808 | * ST * SG * SGmA * SmC * SmA * SmG * SmG | | SSSSO SSSS |
| WV- | mA * Sm5Ceom5CeoGeomC * SG * SA * SC * SC * RC * ST * SC | ACCGCGACCCTCTGGACAGG | SOOOS SSSRS |
| 28809 | * ST * SG * SGmA * SmC * SmA * SmG * SmG | | SSSSO SSSS |
| WV- | mA * Sm5Ceom5CeoGeomC * SG * SA * SC * SC * SC * RT * SC | ACCGCGACCCTCTGGACAGG | SOOOS SSSSR |
| 28810 | * ST * SG * SGmA * SmC * SmA * SmG * SmG | | SSSSO SSSS |
| WV- | mA * Sm5Ceom5CeoGeomC * SG * SA * SC * SC * SC * ST * SC | ACCGCGACCCTCTGGACAGG | SOOOS SSSSS |
| 28811 | * ST * RG * SGmA * SmC * SmA * SmG * SmG | | SSRSO SSSS |
| WV- | mA * Sm5Ceom5CeoGeom5mC * SG * SA * RC * SC * SC * ST * | ACCGCGACCCTCTGGACAGG | SOOOS SRSSS |
| 28812 | SC * ST * SG * SG * RmA * SmC * SmA * SmG * SmG | | SSSSR SSSS |
| WV- | mA * Sm5Ceom5CeoGeom5mC * SG * SA * SC * RC * SC * ST * | ACCGCGACCCTCTGGACAGG | SOOOS SSRSS |
| 28813 | SC * ST * SG * SG * RmA * SmC * SmA * SmG * SmG | | SSSSR SSSS |
| WV- | mA * Sm5Ceom5CeoGeom5mC * SG * SA * SC * SC * RC * ST * | ACCGCGACCCTCTGGACAGG | SOOOS SSSRS |
| 28814 | SC * ST * SG * SG * RmA * SmC * SmA * SmG * SmG | | SSSSR SSSS |
| WV- | mA * Sm5Ceom5CeoGeom5mC * SG * SA * SC * SC * SC * RT * | ACCGCGACCCTCTGGACAGG | SOOOS SSSSR |
| 28815 | SC * ST * SG * SG * RmA * SmC * SmA * SmG * SmG | | SSSSR SSSS |
| WV- | mA * Sm5Ceom5CeoGeom5mC * SG * SA * SC * SC * SC * ST * | ACCGCGACCCTCTGGACAGG | SOOOS SSSSS |
| 28816 | SC * ST * RG * SG * RmA * SmC * SmA * SmG * SmG | | SSRSR SSSS |
| WV- | mA * Sm5Ceom5CeoGeom5mC * SG * SA * RC * SC * SC * ST * | ACCGCGACCCTCTGGACAGG | SOOOS SRSSS |
| 28817 | SC * ST * SG * SG * SmA * SmC * SmA * SmG * SmG | | SSSSS SSSS |
| WV- | mA * Sm5Ceom5CeoGeom5mC * SG * SA * SC * RC * SC * ST * | ACCGCGACCCTCTGGACAGG | SOOOS SSRSS |
| 28818 | SC * ST * SG * SG * SmA * SmC * SmA * SmG * SmG | | SSSSS SSSS |
| WV- | mA * Sm5Ceom5CeoGeom5mC * SG * SA * SC * SC * RC * ST * | ACCGCGACCCTCTGGACAGG | SOOOS SSSRS |
| 28819 | SC * ST * SG * SG * SmA * SmC * SmA * SmG * SmG | | SSSSS SSSS |
| WV- | mA * Sm5Ceom5CeoGeom5mC * SG * SA * SC * SC * SC * RT * | ACCGCGACCCTCTGGACAGG | SOOOS SSSSR |
| 28820 | SC * ST * SG * SG * SmA * SmC * SmA * SmG * SmG | | SSSSS SSSS |
| WV- | mA * Sm5Ceom5CeoGeom5mC * SG * SA * SC * SC * SC * ST * | ACCGCGACCCTCTGGACAGG | SOOOS SSSSS |
| 28821 | SC * ST * RG * SG * SmA * SmC * SmA * SmG * SmG | | SSRSS SSSS |
| WV- | mA * Sm5Ceom5CeoGeom5mC * SG * SA * RC * SC * SC * ST * | ACCGCGACCCTCTGGACAGG | SOOOS SRSSS |
| 28822 | SC * ST * SG * SGmA * SmC * SmA * SmG * SmG | | SSSSO SSSS |
| WV- | mA * Sm5Ceom5CeoGeom5mC * SG * SA * SC * RC * SC * ST * | ACCGCGACCCTCTGGACAGG | SOOOS SSRSS |
| 28823 | SC * ST * SG * SGmA * SmC * SmA * SmG * SmG | | SSSSO SSSS |
| WV- | mA * Sm5Ceom5CeoGeom5mC * SG * SA * SC * SC * RC * ST * | ACCGCGACCCTCTGGACAGG | SOOOS SSSRS |
| 28824 | SC * ST * SG * SGmA * SmC * SmA * SmG * SmG | | SSSSO SSSS |
| WV- | mA * Sm5Ceom5CeoGeom5mC * SG * SA * SC * SC * SC * RT * | ACCGCGACCCTCTGGACAGG | SOOOS SSSSR |
| 28825 | SC * ST * SG * SGmA * SmC * SmA * SmG * SmG | | SSSSO SSSS |
| WV- | mA * Sm5Ceom5CeoGeom5mC * SG * SA * SC * SC * SC * ST * | ACCGCGACCCTCTGGACAGG | SOOOS SSSSS |
| 28826 | SC * ST * RG * SGmA * SmC * SmA * SmG * SmG | | SSRSO SSSS |
| WV- | mA * Sm5Ceom5CeoGeom5Ceo * SG * SA * RC * SC * SC * ST * | ACCGCGACCCTCTGGACAGG | SOOOS SRSSS |
| 28827 | SC * ST * SG * SG * RmA * SmC * SmA * SmG * SmG | | SSSSR SSSS |
| WV- | mA * Sm5Ceom5CeoGeom5Ceo * SG * SA * SC * RC * SC * ST * | ACCGCGACCCTCTGGACAGG | SOOOS SSRSS |
| 28828 | SC * ST * SG * SG * RmA * SmC * SmA * SmG * SmG | | SSSSR SSSS |
| WV- | mA * Sm5Ceom5CeoGeom5Ceo * SG * SA * SC * SC * RC * ST * | ACCGCGACCCTCTGGACAGG | SOOOS SSSRS |
| 28829 | SC * ST * SG * SG * RmA * SmC * SmA * SmG * SmG | | SSSSR SSSS |
| WV- | mA * Sm5Ceom5CeoGeom5Ceo * SG * SA * SC * SC * SC * RT * | ACCGCGACCCTCTGGACAGG | SOOOS SSSSR |
| 28830 | SC * ST * SG * SG * RmA * SmC * SmA * SmG * SmG | | SSSSR SSSS |
| WV- | mA * Sm5Ceom5CeoGeom5Ceo * SG * SA * SC * SC * SC * ST * | ACCGCGACCCTCTGGACAGG | SOOOS SSSSS |
| 28831 | SC * ST * RG * SG * RmA * SmC * SmA * SmG * SmG | | SSRSR SSSS |
| WV- | mA * Sm5Ceom5CeoGeom5Ceo * SG * SA * RC * SC * SC * ST * | ACCGCGACCCTCTGGACAGG | SOOOS SRSSS |
| 28832 | SC * ST * SG * SG * SmA * SmC * SmA * SmG * SmG | | SSSSS SSSS |
| WV- | mA * Sm5Ceom5CeoGeom5Ceo * SG * SA * SC * RC * SC * ST * | ACCGCGACCCTCTGGACAGG | SOOOS SSRSS |
| 28833 | SC * ST * SG * SG * SmA * SmC * SmA * SmG * SmG | | SSSSS SSSS |
| WV- | mA * Sm5Ceom5CeoGeom5Ceo * SG * SA * SC * SC * RC * ST * | ACCGCGACCCTCTGGACAGG | SOOOS SSSRS |
| 28834 | SC * ST * SG * SG * SmA * SmC * SmA * SmG * SmG | | SSSSS SSSS |
| WV- | mA * Sm5Ceom5CeoGeom5Ceo * SG * SA * SC * SC * SC * RT * | ACCGCGACCCTCTGGACAGG | SOOOS SSSSR |
| 28835 | SC * ST * SG * SG * SmA * SmC * SmA * SmG * SmG | | SSSSS SSSS |
| WV- | mA * Sm5Ceom5CeoGeom5Ceo * SG * SA * SC * SC * SC * ST * | ACCGCGACCCTCTGGACAGG | SOOOS SSSSS |
| 28836 | SC * ST * RG * SG * SmA * SmC * SmA * SmG * SmG | | SSRSS SSSS |
| WV- | mA * Sm5Ceom5CeoGeom5Ceo * SG * SA * RC * SC * SC * ST * | ACCGCGACCCTCTGGACAGG | SOOOS SRSSS |
| 28837 | SC * ST * SG * SGmA * SmC * SmA * SmG * SmG | | SSSSO SSSS |
| WV- | mA * Sm5Ceom5CeoGeom5Ceo * SG * SA * SC * RC * SC * ST * | ACCGCGACCCTCTGGACAGG | SOOOS SSRSS |
| 28838 | SC * ST * SG * SGmA * SmC * SmA * SmG * SmG | | SSSSO SSSS |
| WV- | mA * Sm5Ceom5CeoGeom5Ceo * SG * SA * SC * SC * RC * ST * | ACCGCGACCCTCTGGACAGG | SOOOS SSSRS |
| 28839 | SC * ST * SG * SGmA * SmC * SmA * SmG * SmG | | SSSSO SSSS |
| WV- | mA * Sm5Ceom5CeoGeom5Ceo * SG * SA * SC * SC * SC * RT * | ACCGCGACCCTCTGGACAGG | SOOOS SSSSR |
| 28840 | SC * ST * SG * SGmA * SmC * SmA * SmG * SmG | | SSSSO SSSS |
| WV- | mA * Sm5Ceom5CeoGeom5Ceo * SG * SA * SC * SC * SC * ST * | ACCGCGACCCTCTGGACAGG | SOOOS SSSSS |
| 28841 | SC * ST * RG * SGmA * SmC * SmA * SmG * SmG | | SSRSO SSSS |
| WV- | Aeo * Sm5Ceom5CeoGeom5Ceo * SG * SA * RC * SC * SC * ST | ACCGCGACCCTCTGGACAGG | SOOOS SRSSS |
| 28842 | * SC * ST * SG * SG * RmA * SmC * SmA * SmG * SmG | | SSSSR SSSS |
| WV- | Aeo * Sm5Ceom5CeoGeom5Ceo * SG * SA * SC * RC * SC * ST | ACCGCGACCCTCTGGACAGG | SOOOS SSRSS |
| 28843 | * SC * ST * SG * SG * RmA * SmC * SmA * SmG * SmG | | SSSSR SSSS |
| WV- | Aeo * Sm5Ceom5CeoGeom5Ceo * SG * SA * SC * SC * RC * ST | ACCGCGACCCTCTGGACAGG | SOOOS SSSRS |
| 28844 | * SC * ST * SG * SG * RmA * SmC * SmA * SmG * SmG | | SSSSR SSSS |
| WV- | Aeo * Sm5Ceom5CeoGeom5Ceo * SG * SA * SC * SC * SC * RT | ACCGCGACCCTCTGGACAGG | SOOOS SSSSR |
| 28845 | * SC * ST * SG * SG * RmA * SmC * SmA * SmG * SmG | | SSSSR SSSS |
| WV- | Aeo * Sm5Ceom5CeoGeom5Ceo * SG * SA * SC * SC * SC * ST | ACCGCGACCCTCTGGACAGG | SOOOS SSSSS |
| 28846 | * SC * ST * RG * SG * RmA * SmC * SmA * SmG * SmG | | SSRSR SSSS |
| WV- | Aeo * Sm5Ceom5CeoGeom5Ceo * SG * SA * RC * SC * SC * ST | ACCGCGACCCTCTGGACAGG | SOOOS SRSSS |
| 28847 | * SC * ST * SG * SG * SmA * SmC * SmA * SmG * SmG | | SSSSS SSSS |
| WV- | Aeo * Sm5Ceom5CeoGeom5Ceo * SG * SA * SC * RC * SC * ST | ACCGCGACCCTCTGGACAGG | SOOOS SSRSS |
| 28848 | * SC * ST * SG * SG * SmA * SmC * SmA * SmG * SmG | | SSSSS SSSS |
| WV- | Aeo * Sm5Ceom5CeoGeom5Ceo * SG * SA * SC * SC * RC * ST | ACCGCGACCCTCTGGACAGG | SOOOS SSSRS |
| 28849 | * SC * ST * SG * SG * SmA * SmC * SmA * SmG * SmG | | SSSSS SSSS |
| WV- | Aeo * Sm5Ceom5CeoGeom5Ceo * SG * SA * SC * SC * SC * RT | ACCGCGACCCTCTGGACAGG | SOOOS SSSSR |
| 28850 | * SC * ST * SG * SG * SmA * SmC * SmA * SmG * SmG | | SSSSS SSSS |
| WV- | Aeo * Sm5Ceom5CeoGeom5Ceo * SG * SA * SC * SC * SC * ST | ACCGCGACCCTCTGGACAGG | SOOOS SSSSS |
| 28851 | * SC * ST * RG * SG * SmA * SmC * SmA * SmG * SmG | | SSRSS SSSS |
| WV- | Aeo * Sm5Ceom5CeoGeom5Ceo * SG * SA * RC * SC * SC * ST | ACCGCGACCCTCTGGACAGG | SOOOS SRSSS |
| 28852 | * SC * ST * SG * SGmA * SmC * SmA * SmG * SmG | | SSSSO SSSS |
| WV- | Aeo * Sm5Ceom5CeoGeom5Ceo * SG * SA * SC * RC * SC * ST | ACCGCGACCCTCTGGACAGG | SOOOS SSRSS |
| 28853 | * SC * ST * SG * SGmA * SmC * SmA * SmG * SmG | | SSSSO SSSS |
| WV- | Aeo * Sm5Ceom5CeoGeom5Ceo * SG * SA * SC * SC * RC * ST | ACCGCGACCCTCTGGACAGG | SOOOS SSSRS |
| 28854 | * SC * ST * SG * SGmA * SmC * SmA * SmG * SmG | | SSSSO SSSS |
| WV- | Aeo * Sm5Ceom5CeoGeom5Ceo * SG * SA * SC * SC * SC * RT | ACCGCGACCCTCTGGACAGG | SOOOS SSSSR |
| 28855 | * SC * ST * SG * SGmA * SmC * SmA * SmG * SmG | | SSSSO SSSS |
| WV- | Aeo * Sm5Ceom5CeoGeom5Ceo * SG * SA * SC * SC * SC * ST | ACCGCGACCCTCTGGACAGG | SOOOS SSSSS |
| 28856 | * SC * ST * RG * SGmA * SmC * SmA * SmG * SmG | | SSRSO SSSS |
| WV- | mA * Sm5Ceom5CeoGeomU * SG * SA * RC * SC * SC * ST * | ACCGUGACCCTCTGGACAGG | SOOOS SRSSS |
| 28857 | SC * ST * SG * SG * RmA * SmC * SmA * SmG * SmG | | SSSSR SSSS |
| WV- | mA * Sm5Ceom5CeoGeomU * SG * SA * SC * RC * SC * ST * | ACCGUGACCCTCTGGACAGG | SOOOS SSRSS |
| 28858 | SC * ST * SG * SG * RmA * SmC * SmA * SmG * SmG | | SSSSR SSSS |
| WV- | mA * Sm5Ceom5CeoGeomU * SG * SA * SC * SC * RC * ST * | ACCGUGACCCTCTGGACAGG | SOOOS SSSRS |
| 28859 | SC * ST * SG * SG * RmA * SmC * SmA * SmG * SmG | | SSSSR SSSS |
| WV- | mA * Sm5Ceom5CeoGeomU * SG * SA * SC * SC * SC * RT * | ACCGUGACCCTCTGGACAGG | SOOOS SSSSR |
| 28860 | SC * ST * SG * SG * RmA * SmC * SmA * SmG * SmG | | SSSSR SSSS |
| WV- | mA * Sm5Ceom5CeoGeomU * SG * SA * SC * SC * SC * ST * SC | ACCGUGACCCTCTGGACAGG | SOOOS SSSSS |
| 28861 | * ST * RG * SG * RmA * SmC * SmA * SmG * SmG | | SSRSR SSSS |
| WV- | mA * Sm5Ceom5CeoGeomU * SG * SA * RC * SC * SC * ST * | ACCGUGACCCTCTGGACAGG | SOOOS SRSSS |
| 28862 | SC * ST * SG * SG * SmA * SmC * SmA * SmG * SmG | | SSSSS SSSS |
| WV- | mA * Sm5Ceom5CeoGeomU * SG * SA * SC * RC * SC * ST * | ACCGUGACCCTCTGGACAGG | SOOOS SSRSS |
| 28863 | SC * ST * SG * SG * SmA * SmC * SmA * SmG * SmG | | SSSSS SSSS |
| WV- | mA * Sm5Ceom5CeoGeomU * SG * SA * SC * SC * RC * ST * | ACCGUGACCCTCTGGACAGG | SOOOS SSSRS |
| 28864 | SC * ST * SG * SG * SmA * SmC * SmA * SmG * SmG | | SSSSS SSSS |
| WV- | mA * Sm5Ceom5CeoGeomU * SG * SA * SC * SC * SC * RT * | ACCGUGACCCTCTGGACAGG | SOOOS SSSSR |
| 28865 | SC * ST * SG * SG * SmA * SmC * SmA * SmG * SmG | | SSSSS SSSS |
| WV- | mA * Sm5Ceom5CeoGeomU * SG * SA * SC * SC * SC * ST * SC | ACCGUGACCCTCTGGACAGG | SOOOS SSSSS |
| 28866 | * ST * RG * SG * SmA * SmC * SmA * SmG * SmG | | SSRSS SSSS |
| WV- | mA * Sm5Ceom5CeoGeomU * SG * SA * RC * SC * SC * ST * | ACCGUGACCCTCTGGACAGG | SOOOS SRSSS |
| 28867 | SC * ST * SG * SGmA * SmC * SmA * SmG * SmG | | SSSSO SSSS |
| WV- | mA * Sm5Ceom5CeoGeomU * SG * SA * SC * RC * SC * ST * | ACCGUGACCCTCTGGACAGG | SOOOS SSRSS |
| 28868 | SC * ST * SG * SGmA * SmC * SmA * SmG * SmG | | SSSSO SSSS |
| WV- | mA * Sm5Ceom5CeoGeomU * SG * SA * SC * SC * RC * ST * | ACCGUGACCCTCTGGACAGG | SOOOS SSSRS |
| 28869 | SC * ST * SG * SGmA * SmC * SmA * SmG * SmG | | SSSSO SSSS |
| WV- | mA * Sm5Ceom5CeoGeomU * SG * SA * SC * SC * SC * RT * | ACCGUGACCCTCTGGACAGG | SOOOS SSSSR |
| 28870 | SC * ST * SG * SGmA * SmC * SmA * SmG * SmG | | SSSSO SSSS |
| WV- | mA * Sm5Ceom5CeoGeomU * SG * SA * SC * SC * SC * ST * SC | ACCGUGACCCTCTGGACAGG | SOOOS SSSSS |
| 28871 | * ST * RG * SGmA * SmC * SmA * SmG * SmG | | SSRSO SSSS |
| WV- | mG * SmUn001RmU * SmGn001RmA * ST * SC * RT * SG * ST | GUUGATCTGTAGCAGCAGCT | SnRSnRS SRSSR |
| 28878 | * RA * SG * SC * SA * SG * Rm5Ceon001RAeoGeon001Rm5Ceo | | SS SSR nROnRS |
| | * STeo | | |
| WV- | mG * SmUn001RmUmGn001RmA * ST * SC * RT * SG * ST * | GUUGATCTGTAGCAGCAGCT | SnROnRS SRSSR |
| 28879 | RA * SG * SC * SA * SG * Rm5Ceon001RAeoGeon001Rm5Ceo * | | SS SSR nROnRS |
| | STeo | | |
| WV- | mG * SmUn001RmUmGn001RmA * ST * SC * RT * SG * ST * | GUUGATCTGTAGCAGCAGCT | SnROnRS SRSSR |
| 28880 | RA * SG * SC * SA * SG * Rm5CeoAeoGeon001Rm5Ceo * STeo | | SSSSR OOnRS |
| WV- | mG * SmUn001RmU * SmGmA * ST * SC * RT * SG * ST * RA * | GUUGATCTGTAGCAGCAGCT | SnRSOS SRSSR |
| 28881 | SG * SC * SA * SG * Rm5Ceon001RAeoGeon001Rm5Ceo * STeo | | SSSSRnROnRS |
| WV- | Aeo * SGeoAeoGeoGeo * SA * Rm5C * SG * SC * Sm5C * SG * | AGAGGACGCCGTGCAGGGCT | SOOOS RSSSS |
| 29910 | ST * SG * SC * SA * SGeoGeoGeom5Ceo * STeo | | SSSSS OOOS |
| WV- | Aeo * SGeoAeoGeoGeo * SA * Sm5C * RG * SC * Sm5C * SG * | AGAGGACGCCGTGCAGGGCT | SOOOS SRSSS |
| 29911 | ST * SG * SC * SA * SGeoGeoGeom5Ceo * STeo | | SSSSS OOOS |
| WV- | Aeo * SGeoAeoGeoGeo * SA * Sm5C * SG * RC * Sm5C * SG * | AGAGGACGCCGTGCAGGGCT | SOOOS SSRSS |
| 29912 | ST * SG * SC * SA * SGeoGeoGeom5Ceo * STeo | | SSSSS OOOS |
| WV- | Teo * SAeoGeoAeoGeo * SG * SA * Rm5C * SG * SC * Sm5C * | TAGAGGACGCCGTGCAGGGC | SOOOS SRSSS |
| 29913 | SG * ST * SG * SC * SAeoGeoGeoGeo * Sm5Ceo | | SSSSS OOOS |
| WV- | Teo * SAeoGeoAeoGeo * SG * SA * Sm5C * RG * SC * Sm5C * | TAGAGGACGCCGTGCAGGGC | SOOOS SSRSS |
| 29914 | SG * ST * SG * SC * SAeoGeoGeoGeo * 5m5Ceo | | SSSSS OOOS |
| WV- | Teo * SAeoGeoAeoGeo * SG * SA * 5m5C * SG * RC * 5m5C * | TAGAGGACGCCGTGCAGGGC | SOOOS SSSRS |
| 29915 | SG * ST * SG * SC * SAeoGeoGeoGeo * Sm5Ceo | | SSSSS OOOS |
| WV- | Aeo * STeoAeoGeoAeo * SG * SG * SA * Rm5C * SG * SC * | ATAGAGGACGCCGTGCAGGG | SOOOS SSRSS |
| 29916 | Sm5C * SG * ST * SG * Sm5CeoAeoGeoGeo * SGeo | | SSSSS OOOS |
| WV- | Aeo * STeoAeoGeoAeo * SG * SG * SA * Sm5C * RG * SC * | ATAGAGGACGCCGTGCAGGG | SOOOS SSSRS |
| 29917 | Sm5C * SG * ST * SG * Sm5CeoAeoGeoGeo * SGeo | | SSSSS OOOS |
| WV- | Aeo * STeoAeoGeoAeo * SG * SG * SA * Sm5C * SG * RC * | ATAGAGGACGCCGTGCAGGG | SOOOS SSSSR |
| 29918 | Sm5C * SG * ST * SG * Sm5CeoAeoGeoGeo * SGeo | | SSSSS OOOS |
| WV- | m5Ceo * SAeoTeoAeoGeo * SA * SG * SG * SA * Rm5C * SG * | CATAGAGGACGCCGTGCAGG | SOOOS SSSRS |
| 29919 | SC * Sm5C * SG * ST * SGeom5CeoAeoGeo * SGeo | | SSSSS OOOS |
| WV- | m5Ceo * SAeoTeoAeoGeo * SA * SG * SG * SA * Sm5C * RG * | CATAGAGGACGCCGTGCAGG | SOOOS SSSSR |
| 29920 | SC * Sm5C * SG * ST * SGeom5CeoAeoGeo * SGeo | | SSSSS OOOS |
| WV- | m5Ceo * SAeoTeoAeoGeo * SA * SG * SG * SA * Sm5C * SG * | CATAGAGGACGCCGTGCAGG | SOOOS SSSSS |
| 29921 | RC * Sm5C * SG * ST * SGeom5CeoAeoGeo * SGeo | | RSSSS OOOS |
| WV- | Aeo * Sm5CeoAeoTeoAeo * SG * SA * SG * SG * SA * Rm5C * SG | ACATAGAGGACGCCGTGCAG | SOOOS SSSSR |
| 29922 | * SC * Sm5C * SG * STeoGeom5CeoAeo * SGeo | | SSSSS OOOS |
| WV- | Aeo * Sm5CeoAeoTeoAeo * SG * SA * SG * SG * SA * Sm5C * RG | ACATAGAGGACGCCGTGCAG | SOOOS SSSSS |
| 29923 | * SC * Sm5C * SG * STeoGeom5CeoAeo * SGeo | | RSSSS OOOS |
| WV- | Aeo * Sm5CeoAeoTeoAeo * SG * SA * SG * SG * SA * Sm5C * SG | ACATAGAGGACGCCGTGCAG | SOOOS SSSSS |
| 29924 | * RC * Sm5C * SG * STeoGeom5CeoAeo * SGeo | | SRSSS OOOS |
| WV- | m5Ceo * SAeom5CeoAeoTeo * SA * SG * SA * SG * SG * SA * | CACATAGAGGACGCCGTGCA | SOOOS SSSSS |
| 29925 | Rm5C * SG * SC * Sm5C * SGeoTeoGeom5Ceo * SAeo | | RSSSS OOOS |
| WV- | m5Ceo * SAeom5CeoAeoTeo * SA * SG * SA * SG * SG * SA * | CACATAGAGGACGCCGTGCA | SOOOS SSSSS |
| 29926 | Sm5C * RG * SC * Sm5C * SGeoTeoGeom5Ceo * SAeo | | SRSSS OOOS |
| WV- | m5Ceo * SAeom5CeoAeoTeo * SA * SG * SA * SG * SG * SA * | CACATAGAGGACGCCGTGCA | SOOOS SSSSS |
| 29927 | Sm5C * SG * RC * Sm5C * SGeoTeoGeom5Ceo * SAeo | | SSRSS OOOS |
| WV- | Geo * Sm5CeoAeom5CeoAeo * ST * SA * SG * SA * SG * SG * SA | GCACATAGAGGACGCCGTGC | SOOOS SSSSS |
| 29928 | * Rm5C * SG * SC * Sm5CeoGeoTeoGeo * Sm5Ceo | | SRSSS OOOS |
| WV- | Geo * Sm5CeoAeom5CeoAeo * ST * SA * SG * SA * SG * SG * SA | GCACATAGAGGACGCCGTGC | SOOOS SSSSS |
| 29929 | * Sm5C * RG * SC * Sm5CeoGeoTeoGeo * Sm5Ceo | | SSRSS OOOS |
| WV- | Geo * Sm5CeoAeom5CeoAeo * ST * SA * SG * SA * SG * SG * SA | GCACATAGAGGACGCCGTGC | SOOOS SSSSS |
| 29930 | * Sm5C * SG * RC * Sm5CeoGeoTeoGeo * Sm5Ceo | | SSSRS OOOS |
| WV- | Aeo * SGeoAeoGeoGeo * SA * Rm5C * SG * SC * Sm5C * SG * ST | AGAGGACGCCGTGCAGGGCU | SOOOS RSSSS |
| 29931 | * SG * SC * SA * SmG * SmG * SmG * SmC * SmU | | SSSSS SSSS |
| WV- | Aeo * SGeoAeoGeoGeo * SA * 5m5C * RG * SC * 5m5C * SG * ST | AGAGGACGCCGTGCAGGGCU | SOOOS SRSSS |
| 29932 | * SG * SC * SA * SmG * SmG * SmG * SmC * SmU | | SSSSS SSSS |
| WV- | Aeo * SGeoAeoGeoGeo * SA * 5m5C * SG * RC * 5m5C * SG * ST | AGAGGACGCCGTGCAGGGCU | SOOOS SSRSS |
| 29933 | * SG * SC * SA * SmG * SmG * SmG * SmC * SmU | | SSSSS SSSS |
| WV- | Teo * SAeoGeoAeoGeo * SG * SA * Rm5C * SG * SC * 5m5C * SG | TAGAGGACGCCGTGCAGGGC | SOOOS SRSSS |
| 29934 | * ST * SG * SC * SmA * SmG * SmG * SmG * SmC | | SSSSS SSSS |
| WV- | Teo * SAeoGeoAeoGeo * SG * SA * 5m5C * RG * SC * 5m5C * SG | TAGAGGACGCCGTGCAGGGC | SOOOS SSRSS |
| 29935 | * ST * SG * SC * SmA * SmG * SmG * SmG * SmC | | SSSSS SSSS |
| WV- | Teo * SAeoGeoAeoGeo * SG * SA * Sm5C * SG * RC * Sm5C * SG | TAGAGGACGCCGTGCAGGGC | SOOOS SSSRS |
| 29936 | * ST * SG * SC * SmA * SmG * SmG * SmG * SmC | | SSSSS SSSS |
| WV- | Aeo * STeoAeoGeoAeo * SG * SG * SA * Rm5C * SG * SC * | ATAGAGGACGCCGTGCAGGG | SOOOS SSRSS |
| 29937 | Sm5 * SG * ST * SG * SmC * SmA * SmG * SmG * SmG | | SSSSS SSSS |
| WV- | Aeo * STeoAeoGeoAeo * SG * SG * SA * Sm5C * RG * SC * | ATAGAGGACGCCGTGCAGGG | SOOOS SSSRS |
| 29938 | Sm5 * SG * ST * SG * SmC * SmA * SmG * SmG * SmG | | SSSSS SSSS |
| WV- | Aeo * STeoAeoGeoAeo * SG * SG * SA * Sm5C * SG * RC * | ATAGAGGACGCCGTGCAGGG | SOOOS SSSSR |
| 29939 | Sm5 * SG * ST * SG * SmC * SmA * SmG * SmG * SmG | | SSSSS SSSS |
| WV- | m5Ceo * SAeoTeoAeoGeo * SA * SG * SG * SA * Rm5C * SG * SC | CATAGAGGACGCCGTGCAGG | SOOOS SSSRS |
| 29940 | * Sm5C * SG * ST * SmG * SmC * SmA * SmG * SmG | | SSSSS SSSS |
| WV- | m5Ceo * SAeoTeoAeoGeo * SA * SG * SG * SA * Sm5C * RG * SC | CATAGAGGACGCCGTGCAGG | SOOOS SSSSR |
| 29941 | * Sm5C * SG * ST * SmG * SmC * SmA * SmG * SmG | | SSSSS SSSS |
| WV- | m5Ceo * SAeoTeoAeoGeo * SA * SG * SG * SA * Sm5C * SG * RC | CATAGAGGACGCCGTGCAGG | SOOOS SSSSS |
| 29942 | * Sm5C * SG * ST * SmG * SmC * SmA * SmG * SmG | | RSSSS SSSS |
| WV- | Aeo * Sm5CeoAeoTeoAeo * SG * SA * SG * SG * SA * Rm5C * SG | ACATAGAGGACGCCGUGCAG | SOOOS SSSSR |
| 29943 | * SC * Sm5C * SG * SmU * SmG * SmC * SmA * SmG | | SSSSS SSSS |
| WV- | Aeo * Sm5CeoAeoTeoAeo * SG * SA * SG * SG * SA * Sm5C * RG | ACATAGAGGACGCCGUGCAG | SOOOS SSSSS |
| 29944 | * SC * Sm5C * SG * SmU * SmG * SmC * SmA * SmG | | RSSSS SSSS |
| WV- | Aeo * Sm5CeoAeoTeoAeo * SG * SA * SG * SG * SA * Sm5C * SG | ACATAGAGGACGCCGUGCAG | SOOOS SSSSS |
| 29945 | * RC * Sm5C * SG * SmU * SmG * SmC * SmA * SmG | | SRSSS SSSS |
| WV- | m5Ceo * SAeom5CeoAeoTeo * SA * SG * SA * SG * SG * SA * | CACATAGAGGACGCCGUGCA | SOOOS SSSSS |
| 29946 | Rm5C * SG * SC * Sm5C * SmG * SmU * SmG * SmC * SmA | | RSSSS SSSS |
| WV- | m5Ceo * SAeom5CeoAeoTeo * SA * SG * SA * SG * SG * SA * | CACATAGAGGACGCCGUGCA | SOOOS SSSSS |
| 29947 | Sm5C * RG * SC * Sm5C * SmG * SmU * SmG * SmC * SmA | | SRSSS SSSS |
| WV- | m5Ceo * SAeom5CeoAeoTeo * SA * SG * SA * SG * SG * SA * | CACATAGAGGACGCCGUGCA | SOOOS SSSSS |
| 29948 | Sm5C * SG * RC * Sm5C * SmG * SmU * SmG * SmC * SmA | | SSRSS SSSS |
| WV- | Geo * Sm5CeoAeom5CeoAeo * ST * SA * SG * SA * SG * SG * SA | GCACATAGAGGACGCCGUGC | SOOOS SSSSS |
| 29949 | * Rm5C * SG * SC * Sm5mC * SmG * SmU * SmG * SmC | | SRSSS SSSS |
| WV- | Geo * Sm5CeoAeom5CeoAeo * ST * SA * SG * SA * SG * SG * SA | GCACATAGAGGACGCCGUGC | SOOOS SSSSS |
| 29950 | * Sm5C * RG * SC * Sm5mC * SmG * SmU * SmG * SmC | | SSRSS SSSS |
| WV- | Geo * Sm5CeoAeom5CeoAeo * ST * SA * SG * SA * SG * SG * SA | GCACATAGAGGACGCCGUGC | SOOOS SSSSS |
| 29951 | * Sm5C * SG * RC * Sm5mC * SmG * SmU * SmG * SmC | | SSSRS SSSS |
| WV- | mA * SmG * SmA * SmG * SmG * SA * Rm5C * SG * SC * Sm5C * | AGAGGACGCCGTGCAGGGCT | SSSSS RSSSS |
| 29952 | SG * ST * SG * SC * SA * SGeoGeoGeom5Ceo * STeo | | SSSSS OOOS |
| WV- | mA * SmG * SmA * SmG * SmG * SA * Sm5C * RG * SC * Sm5C * | AGAGGACGCCGTGCAGGGCT | SSSSS SRSSS |
| 29953 | SG * ST * SG * SC * SA * SGeoGeoGeom5Ceo * STeo | | SSSSS OOOS |
| WV- | mA * SmG * SmA * SmG * SmG * SA * Sm5C * SG * RC * Sm5C * | AGAGGACGCCGTGCAGGGCT | SSSSS SSRSS |
| 29954 | SG * ST * SG * SC * SA * SGeoGeoGeom5Ceo * STeo | | SSSSS OOOS |
| WV- | mU * SmA * SmG * SmA * SmG * SG * SA * Rm5C * SG * SC * | UAGAGGACGCCGTGCAGGGC | SSSSS SRSSS |
| 29955 | Sm5C * SG * ST * SG * SC * SAeoGeoGeoGeo * Sm5Ceo | | SSSSS OOOS |
| WV- | mU * SmA * SmG * SmA * SmG * SG * SA * Sm5C * RG * SC * | UAGAGGACGCCGTGCAGGGC | SSSSS SSRSS |
| 29956 | Sm5C * SG * ST * SG * SC * SAeoGeoGeoGeo * Sm5Ceo | | SSSSS OOOS |
| WV- | mU * SmA * SmG * SmA * SmG * SG * SA * Sm5C * SG * RC * | UAGAGGACGCCGTGCAGGGC | SSSSS SSSRS |
| 29957 | Sm5C * SG * ST * SG * SC * SAeoGeoGeoGeo * Sm5Ceo | | SSSSS OOOS |
| WV- | mA * SmU * SmA * SmG * SmA * SG * SG * SA * Rm5C * SG * SC | AUAGAGGACGCCGTGCAGGG | SSSSS SSRSS |
| 29958 | * Sm5C * SG * ST * SG * Sm5CeoAeoGeoGeo * SGeo | | SSSSS OOOS |
| WV- | mA * SmU * SmA * SmG * SmA * SG * SG * SA * Sm5C * RG * SC | AUAGAGGACGCCGTGCAGGG | SSSSS SSSRS |
| 29959 | * Sm5C * SG * ST * SG * Sm5CeoAeoGeoGeo * SGeo | | SSSSS OOOS |
| WV- | mA * SmU * SmA * SmG * SmA * SG * SG * SA * Sm5C * SG * RC | AUAGAGGACGCCGTGCAGGG | SSSSS SSSSR |
| 29960 | * Sm5C * SG * ST * SG * Sm5CeoAeoGeoGeo * SGeo | | SSSSS OOOS |
| WV- | mC * SmA * SmU * SmA * SmG * SA * SG * SG * SA * Rm5C * SG | CAUAGAGGACGCCGTGCAGG | SSSSS SSSRS |
| 29961 | * SC * Sm5C * SG * ST * SGeom5CeoAeoGeo * SGeo | | SSSSS OOOS |
| WV- | mC * SmA * SmU * SmA * SmG * SA * SG * SG * SA * Sm5C * RG | CAUAGAGGACGCCGTGCAGG | SSSSS SSSSR |
| 29962 | * SC * Sm5C * SG * ST * SGeom5CeoAeoGeo * SGeo | | SSSSS OOOS |
| WV- | mC * SmA * SmU * SmA * SmG * SA * SG * SG * SA * Sm5C * SG | CAUAGAGGACGCCGTGCAGG | SSSSS SSSSS |
| 29963 | * RC * Sm5C * SG * ST * SGeom5CeoAeoGeo * SGeo | | RSSSS OOOS |
| WV- | mA * SmC * SmA * SmU * SmA * SG * SA * SG * SG * SA * Rm5C | ACAUAGAGGACGCCGTGCAG | SSSSS SSSSR |
| 29964 | * SG * SC * Sm5C * SG * STeoGeom5CeoAeo * SGeo | | SSSSS OOOS |
| WV- | mA * SmC * SmA * SmU * SmA * SG * SA * SG * SG * SA * Sm5C | ACAUAGAGGACGCCGTGCAG | SSSSS SSSSS |
| 29965 | * RG * SC * Sm5C * SG * STeoGeom5CeoAeo * SGeo | | RSSSS OOOS |
| WV- | mA * SmC * SmA * SmU * SmA * SG * SA * SG * SG * SA * Sm5C | ACAUAGAGGACGCCGTGCAG | SSSSS SSSSS |
| 29966 | * SG * RC * Sm5C * SG * STeoGeom5CeoAeo * SGeo | | SRSSS OOOS |
| WV- | mC * SmA * SmC * SmA * SmU * SA * SG * SA * SG * SG * SA * | CACAUAGAGGACGCCGTGCA | SSSSS SSSSS |
| 29967 | Rm5C * SG * SC * Sm5C * SGeoTeoGeom5Ceo * SAeo | | RSSSS OOOS |
| WV- | mC * SmA * SmC * SmA * SmU * SA * SG * SA * SG * SG * SA * | CACAUAGAGGACGCCGTGCA | SSSSS SSSSS |
| 29968 | Sm5C * RG * SC * Sm5C * SGeoTeoGeom5Ceo * SAeo | | SRSSS OOOS |
| WV- | mC * SmA * SmC * SmA * SmU * SA * SG * SA * SG * SG * SA * | CACAUAGAGGACGCCGTGCA | SSSSS SSSSS |
| 29969 | Sm5C * SG * RC * Sm5C * SGeoTeoGeom5Ceo * SAeo | | SSRSS OOOS |
| WV- | mG * SmC * SmA * SmC * SmA * ST * SA * SG * SA * SG * SG * | GCACATAGAGGACGCCGTGC | SSSSS SSSSS |
| 29970 | SA * Rm5C * SG * SC * Sm5CeoGeoTeoGeo * 5m5Ceo | | SRSSS OOOS |
| WV- | mG * SmC * SmA * SmC * SmA * ST * SA * SG * SA * SG * SG * | GCACATAGAGGACGCCGTGC | SSSSS SSSSS |
| 29971 | SA * Sm5C * RG * SC * Sm5CeoGeoTeoGeo * Sm5Ceo | | SSRSS OOOS |
| WV- | mG * SmC * SmA * SmC * SmA * ST * SA * SG * SA * SG * SG * | GCACATAGAGGACGCCGTGC | SSSSS SSSSS |
| 29972 | SA * Sm5C * SG * RC * Sm5CeoGeoTeoGeo * Sm5Ceo | | SSSRS OOOS |
| WV- | Aeo * SGeon001Aeon001Geon001Geo * SA * Rm5C * SG * SC * | AGAGGACGCCGTGCAGGGCT | SnXnXnXS RSSSS |
| 29973 | 5m5C * SG * ST * SG * SC * SA * SGeoGeoGeom5Ceo * STeo | | SSSSS OOOS |
| WV- | Aeo * SGeon001Aeon001Geon001Geo * SA * 5m5C * RG * SC * | AGAGGACGCCGTGCAGGGCT | SnXnXnXS SRSSS |
| 29974 | 5m5C * SG * ST * SG * SC * SA * SGeoGeoGeom5Ceo * STeo | | SSSSS OOOS |
| WV- | Aeo * SGeon001Aeon001Geon001Geo * SA * 5m5C * SG * RC * | AGAGGACGCCGTGCAGGGCT | SnXnXnXS SSRSS |
| 29975 | Sm5C * SG * ST * SG * SC * SA * SGeoGeoGeom5Ceo * STeo | | SSSSS OOOS |
| WV- | Teo * SAeon001Geon001Aeon001Geo * SG * SA * Rm5C * SG * SC | TAGAGGACGCCGTGCAGGGC | SnXnXnXS SRSSS |
| 29976 | * Sm5C * SG * ST * SG * SC * SAeoGeoGeoGeo * Sm5Ceo | | SSSSS OOOS |
| WV- | Teo * SAeon001Geon001Aeon001Geo * SG * SA * Sm5C * RG * SC | TAGAGGACGCCGTGCAGGGC | SnXnXnXS SSRSS |
| 29977 | * Sm5C * SG * ST * SG * SC * SAeoGeoGeoGeo * Sm5Ceo | | SSSSS OOOS |
| WV- | Teo * SAeon001Geon001Aeon001Geo * SG * SA * Sm5C * SG * RC | TAGAGGACGCCGTGCAGGGC | SnXnXnXS SSSRS |
| 29978 | * Sm5C * SG * ST * SG * SC * SAeoGeoGeoGeo * Sm5Ceo | | SSSSS OOOS |
| WV- | Aeo * STeon001Aeon001Geon001Aeo * SG * SG * SA * Rm5C * SG | ATAGAGGACGCCGTGCAGGG | SnXnXnXS SSRSS |
| 29979 | * SC * Sm5C * SG * ST * SG * Sm5CeoAeoGeoGeo * SGeo | | SSSSS OOOS |
| WV- | Aeo * STeon001Aeon001Geon001Aeo * SG * SG * SA * Sm5C * RG | ATAGAGGACGCCGTGCAGGG | SnXnXnXS SSSRS |
| 29980 | * SC * Sm5C * SG * ST * SG * Sm5CeoAeoGeoGeo * SGeo | | SSSSS OOOS |
| WV- | Aeo * STeon001Aeon001Geon001Aeo * SG * SG * SA * Sm5C * SG | ATAGAGGACGCCGTGCAGGG | SnXnXnXS SSSSR |
| 29981 | * RC * Sm5C * SG * ST * SG * Sm5CeoAeoGeoGeo * SGeo | | SSSSS OOOS |
| WV- | m5Ceo * SAeon001Teon001Aeon001Geo * SA * SG * SG * SA * | CATAGAGGACGCCGTGCAGG | SnXnXnXS SSSRS |
| 29982 | Rm5C * SG * SC * Sm5C * SG * ST * SGeom5CeoAeoGeo * SGeo | | SSSSS OOOS |
| WV- | m5Ceo * SAeon001Teon001Aeon001Geo * SA * SG * SG * SA * | CATAGAGGACGCCGTGCAGG | SnXnXnXS SSSSR |
| 29983 | Sm5C * RG * SC * Sm5C * SG * ST * SGeom5CeoAeoGeo * SGeo | | SSSSS OOOS |
| WV- | m5Ceo * SAeon001Teon001Aeon001Geo * SA * SG * SG * SA * | CATAGAGGACGCCGTGCAGG | SnXnXnXS SSSSS |
| 29984 | Sm5C * SG * RC * Sm5C * SG * ST * SGeom5CeoAeoGeo * SGeo | | RSSSS OOOS |
| WV- | Aeo * Sm5Ceon001Aeon001Teon001Aeo * SG * SA * SG * SG * SA | ACATAGAGGACGCCGTGCAG | SnXnXnXS SSSSR |
| 29985 | * Rm5C * SG * SC * Sm5C * SG * STeoGeom5CeoAeo * SGeo | | SSSSS OOOS |
| WV- | Aeo * Sm5Ceon001Aeon001Teon001Aeo * SG * SA * SG * SG * SA | ACATAGAGGACGCCGTGCAG | SnXnXnXS SSSSS |
| 29986 | * Sm5C * RG * SC * Sm5C * SG * STeoGeom5CeoAeo * SGeo | | RSSSS OOOS |
| WV- | Aeo * Sm5Ceon001Aeon001Teon001Aeo * SG * SA * SG * SG * SA | ACATAGAGGACGCCGTGCAG | SnXnXnXS SSSSS |
| 29987 | * Sm5C * SG * RC * Sm5C * SG * STeoGeom5CeoAeo * SGeo | | SRSSS OOOS |
| WV- | m5Ceo * SAeon001m5Ceon001Aeon001Teo * SA * SG * SA * SG * | CACATAGAGGACGCCGTGCA | SnXnXnXS SSSSS |
| 29988 | SG * SA * Rm5C * SG * SC * Sm5C * SGeoTeoGeom5Ceo * SAeo | | RSSSS OOOS |
| WV- | m5Ceo * SAeon001m5Ceon001Aeon001Teo * SA * SG * SA * SG * | CACATAGAGGACGCCGTGCA | SnXnXnXS SSSSS |
| 29989 | SG * SA * Sm5C * RG * SC * Sm5C * SGeoTeoGeom5Ceo * SAeo | | SRSSS OOOS |
| WV- | m5Ceo * SAeon001m5Ceon001Aeon001Teo * SA * SG * SA * SG * | CACATAGAGGACGCCGTGCA | SnXnXnXS SSSSS |
| 29990 | SG * SA * Sm5C * SG * RC * Sm5C * SGeoTeoGeom5Ceo * SAeo | | SSRSS OOOS |
| WV- | Geo * Sm5Ceon001Aeon001m5Ceon001Aeo * ST * SA * SG * SA * | GCACATAGAGGACGCCGTGC | SnXnXnXS SSSSS |
| 29991 | SG * SG * SA * Rm5C * SG * SC * Sm5CeoGeoTeoGeo * Sm5Ceo | | SRSSS OOOS |
| WV- | Geo * Sm5Ceon001Aeon001m5Ceon001Aeo * ST * SA * SG * SA * | GCACATAGAGGACGCCGTGC | SnXnXnXS SSSSS |
| 29992 | SG * SG * SA * Sm5C * RG * SC * Sm5CeoGeoTeoGeo * Sm5Ceo | | SSRSS OOOS |
| WV- | Geo * Sm5Ceon001Aeon001m5Ceon001Aeo * ST * SA * SG * SA * | GCACATAGAGGACGCCGTGC | SnXnXnXS SSSSS |
| 29993 | SG * SG * SA * Sm5C * SG * RC * Sm5CeoGeoTeoGeo * Sm5Ceo | | SSSRS OOOS |
| WV- | Aeo * SGeoAeoGeoGeo * SA * Rm5C * SG * SC * Sm5C * SG * ST | AGAGGACGCCGTGCAGGGCT | SOOOS RSSSS |
| 29994 | * SG * SC * SA * SGeon001Geon001Geon001m5Ceo * STeo | | SSSSS nXnXnXS |
| WV- | Aeo * SGeoAeoGeoGeo * SA * Sm5C * RG * SC * Sm5C * SG * ST | AGAGGACGCCGTGCAGGGCT | SOOOS SRSSS |
| 29995 | * SG * SC * SA * SGeon001Geon001Geon001m5Ceo * STeo | | SSSSS nXnXnXS |
| WV- | Aeo * SGeoAeoGeoGeo * SA * Sm5C * SG * RC * Sm5C * SG * ST | AGAGGACGCCGTGCAGGGCT | SOOOS SSRSS |
| 29996 | * SG * SC * SA * SGeon001Geon001Geon001m5Ceo * STeo | | SSSSS nXnXnXS |
| WV- | Teo * SAeoGeoAeoGeo * SG * SA * Rm5C * SG * SC * Sm5C * SG | TAGAGGACGCCGTGCAGGGC | SOOOS SRSSS |
| 29997 | * ST * SG * SC * SAeon001Geon001Geon001Geo * Sm5Ceo | | SSSSS nXnXnXS |
| WV- | Teo * SAeoGeoAeoGeo * SG * SA * Sm5C * RG * SC * Sm5C * SG | TAGAGGACGCCGTGCAGGGC | SOOOS SSRSS |
| 29998 | * ST * SG * SC * SAeon001Geon001Geon001Geo * Sm5Ceo | | SSSSS nXnXnXS |
| WV- | Teo * SAeoGeoAeoGeo * SG * SA * Sm5C * SG * RC * Sm5C * SG | TAGAGGACGCCGTGCAGGGC | SOOOS SSSRS |
| 29999 | * ST * SG * SC * SAeon001Geon001Geon001Geo * Sm5Ceo | | SSSSS nXnXnXS |
| WV- | Aeo * STeoAeoGeoAeo * SG * SG * SA * Rm5C * SG * SC * Sm5C | ATAGAGGACGCCGTGCAGGG | SOOOS SSRSS |
| 30000 | * SG * ST * SG * Sm5Ceon001Aeon001Geon001Geo * SGeo | | SSSSS nXnXnXS |
| WV- | Aeo * STeoAeoGeoAeo * SG * SG * SA * Sm5C * RG * SC * Sm5C | ATAGAGGACGCCGTGCAGGG | SOOOS SSSRS |
| 30001 | * SG * ST * SG * Sm5Ceon001Aeon001Geon001Geo * SGeo | | SSSSS nXnXnXS |
| WV- | Aeo * STeoAeoGeoAeo * SG * SG * SA * Sm5C * SG * RC * Sm5C | ATAGAGGACGCCGTGCAGGG | SOOOS SSSSR |
| 30002 | * SG * ST * SG * Sm5Ceon001Aeon001Geon001Geo * SGeo | | SSSSS nXnXnXS |
| WV- | m5Ceo * SAeoTeoAeoGeo * SA * SG * SG * SA * Rm5C * SG * SC | CATAGAGGACGCCGTGCAGG | SOOOS SSSRS |
| 30003 | * Sm5C * SG * ST * SGeon001m5Ceon001Aeon001Geo * SGeo | | SSSSS nXnXnXS |
| WV- | m5Ceo * SAeoTeoAeoGeo * SA * SG * SG * SA * Sm5C * RG * SC | CATAGAGGACGCCGTGCAGG | SOOOS SSSSR |
| 30004 | * Sm5C * SG * ST * SGeon001m5Ceon001Aeon001Geo * SGeo | | SSSSS nXnXnXS |
| WV- | m5Ceo * SAeoTeoAeoGeo * SA * SG * SG * SA * Sm5C * SG * RC | CATAGAGGACGCCGTGCAGG | SOOOS SSSSS |
| 30005 | * Sm5C * SG * ST * SGeon001m5Ceon001Aeon001Geo * SGeo | | RSSSS nXnXnXS |
| WV- | Aeo * Sm5CeoAeoTeoAeo * SG * SA * SG * SG * SA * Rm5C * SG | ACATAGAGGACGCCGTGCAG | SOOOS SSSSR |
| 30006 | * SC * Sm5C * SG * STeon001Geon001m5Ceon001Aeo * SGeo | | SSSSS nXnXnXS |
| WV- | Aeo * Sm5CeoAeoTeoAeo * SG * SA * SG * SG * SA * Sm5C * RG | ACATAGAGGACGCCGTGCAG | SOOOS SSSSS |
| 30007 | * SC * Sm5C * SG * STeon001Geon001m5Ceon001Aeo * SGeo | | RSSSS nXnXnXS |
| WV- | Aeo * Sm5CeoAeoTeoAeo * SG * SA * SG * SG * SA * Sm5C * SG | ACATAGAGGACGCCGTGCAG | SOOOS SSSSS |
| 30008 | * RC * Sm5C * SG * STeon001Geon001m5Ceon001Aeo * SGeo | | SRSSS nXnXnXS |
| WV- | m5Ceo * SAeom5CeoAeoTeo * SA * SG * SA * SG * SG * SA * Rm5C | CACATAGAGGACGCCGTGCA | SOOOS SSSSS |
| 30009 | * SG * SC * Sm5C * SGeon001Teon001Geon001m5Ceo * SAeo | | RSSSS nXnXnXS |
| WV- | m5Ceo * SAeom5CeoAeoTeo * SA * SG * SA * SG * SG * SA * Sm5C | CACATAGAGGACGCCGTGCA | SOOOS SSSSS |
| 30010 | * RG * SC * Sm5C * SGeon001Teon001Geon001m5Ceo * SAeo | | SRSSS nXnXnXS |
| WV- | m5Ceo * SAeom5CeoAeoTeo * SA * SG * SA * SG * SG * SA * Sm5C | CACATAGAGGACGCCGTGCA | SOOOS SSSSS |
| 30011 | * SG * RC * Sm5C * SGeon001Teon001Geon001m5Ceo * SAeo | | SSRSS nXnXnXS |
| WV- | Geo * Sm5CeoAeom5CeoAeo * ST * SA * SG * SA * SG * SG * SA * | GCACATAGAGGACGCCGTGC | SOOOS SSSSS |
| 30012 | Rm5C * SG * SC * Sm5Ceon001Geon001Teon001Geo * Sm5Ceo | | SRSSS nXnXnXS |
| WV- | Geo * Sm5CeoAeom5CeoAeo * ST * SA * SG * SA * SG * SG * SA * | GCACATAGAGGACGCCGTGC | SOOOS SSSSS |
| 30013 | Sm5C * RG * SC * Sm5Ceon001Geon001Teon001Geo * Sm5Ceo | | SSRSS nXnXnXS |
| WV- | Geo * Sm5CeoAeom5CeoAeo * ST * SA * SG * SA * SG * SG * SA * | GCACATAGAGGACGCCGTGC | SOOOS SSSSS |
| 30014 | Sm5C * SG * RC * Sm5Ceon001Geon001Teon001Geo * Sm5Ceo | | SSSRS nXnXnXS |
| WV- | Aeo * SGeon001Aeon001Geon001Geo * SA * Rm5C * SG * SC * | AGAGGACGCCGTGCAGGGCU | SnXnXnXS RSSSS |
| 30015 | Sm5C * SG * ST * SG * SC * SA * SmG * SmG * SmG * SmC * SmU | | SSSSS SSSS |
| WV- | Aeo * SGeon001Aeon001Geon001Geo * SA * Sm5C * RG * SC * | AGAGGACGCCGTGCAGGGCU | SnXnXnXS SRSSS |
| 30016 | Sm5C * SG * ST * SG * SC * SA * SmG * SmG * SmG * SmC * SmU | | SSSSS SSSS |
| WV- | Aeo * SGeon001Aeon001Geon001Geo * SA * Sm5C * SG * RC * | AGAGGACGCCGTGCAGGGCU | SnXnXnXS SSRSS |
| 30017 | Sm5C * SG * ST * SG * SC * SA * SmG * SmG * SmG * SmC * SmU | | SSSSS SSSS |
| WV- | Teo * SAeon001Geon001Aeon001Geo * SG * SA * Rm5C * SG * SC * | TAGAGGACGCCGTGCAGGGC | SnXnXnXS SRSSS |
| 30018 | Sm5C * SG * ST * SG * SC * SmA * SmG * SmG * SmG * SmC | | SSSSS SSSS |
| WV- | Teo * SAeon001Geon001Aeon001Geo * SG * SA * Sm5C * RG * SC * | TAGAGGACGCCGTGCAGGGC | SnXnXnXS SSRSS |
| 30019 | Sm5C * SG * ST * SG * SC * SmA * SmG * SmG * SmG * SmC | | SSSSS SSSS |
| WV- | Teo * SAeon001Geon001Aeon001Geo * SG * SA * Sm5C * SG * RC * | TAGAGGACGCCGTGCAGGGC | SnXnXnXS SSSRS |
| 30020 | Sm5C * SG * ST * SG * SC * SmA * SmG * SmG * SmG * SmC | | SSSSS SSSS |
| WV- | Aeo * STeon001Aeon001Geon001Aeo * SG * SG * SA * Rm5C * SG * | ATAGAGGACGCCGTGCAGGG | SnXnXnXS SSRSS |
| 30021 | SC * Sm5C * SG * ST * SG * SmC * SmA * SmG * SmG * SmG | | SSSSS SSSS |
| WV- | Aeo * STeon001Aeon001Geon001Aeo * SG * SG * SA * Sm5C * RG * | ATAGAGGACGCCGTGCAGGG | SnXnXnXS SSSRS |
| 30022 | SC * Sm5C * SG * ST * SG * SmC * SmA * SmG * SmG * SmG | | SSSSS SSSS |
| WV- | Aeo * STeon001Aeon001Geon001Aeo * SG * SG * SA * Sm5C * SG * | ATAGAGGACGCCGTGCAGGG | SnXnXnXS SSSSR |
| 30023 | RC * Sm5C * SG * ST * SG * SmC * SmA * SmG * SmG * SmG | | SSSSS SSSS |
| WV- | m5Ceo * SAeon001Teon001Aeon001Geo * SA * SG * SG * SA * | CATAGAGGACGCCGTGCAGG | SnXnXnXS SSSRS |
| 30024 | Rm5C * SG * SC * Sm5C * SG * ST * SmG * SmC * SmA * SmG * | | SSSSS SSSS |
| | SmG | | |
| WV- | m5Ceo * SAeon001Teon001Aeon001Geo * SA * SG * SG * SA * Sm5C | CATAGAGGACGCCGTGCAGG | SnXnXnXS SSSSR |
| 30025 | * RG * SC * Sm5C * SG * ST * SmG * SmC * SmA * SmG * SmG | | SSSSS SSSS |
| WV- | m5Ceo * SAeon001Teon001Aeon001Geo * SA * SG * SG * SA * Sm5C | CATAGAGGACGCCGTGCAGG | SnXnXnXS SSSSS |
| 30026 | * SG * RC * Sm5C * SG * ST * SmG * SmC * SmA * SmG * SmG | | RSSSS SSSS |
| WV- | Aeo * Sm5Ceon001Aeon001Teon001Aeo * SG * SA * SG * SG * SA * | ACATAGAGGACGCCGUGCAG | SnXnXnXS SSSSR |
| 30027 | Rm5C * SG * SC * Sm5C * SG * SmU * SmG * SmC * SmA * SmG | | SSSSS SSSS |
| WV- | Aeo * Sm5Ceon001Aeon001Teon001Aeo * SG * SA * SG * SG * SA * | ACATAGAGGACGCCGUGCAG | SnXnXnXS SSSSS |
| 30028 | Sm5C * RG * SC * Sm5C * SG * SmU * SmG * SmC * SmA * SmG | | RSSSS SSSS |
| WV- | Aeo * Sm5Ceon001Aeon001Teon001Aeo * SG * SA * SG * SG * SA * | ACATAGAGGACGCCGUGCAG | SnXnXnXS SSSSS |
| 30029 | Sm5C * SG * RC * Sm5C * SG * SmU * SmG * SmC * SmA * SmG | | SRSSS SSSS |
| WV- | m5Ceo * SAeon001m5Ceon001Aeon001Teo * SA * SG * SA * SG * SG | CACATAGAGGACGCCGUGCA | SnXnXnXS SSSSS |
| 30030 | * SA * Rm5C * SG * SC * Sm5C * SmG * SmU * SmG * SmC * SmA | | RSSSS SSSS |
| WV- | m5Ceo * SAeon001m5Ceon001Aeon001Teo * SA * SG * SA * SG * SG | CACATAGAGGACGCCGUGCA | SnXnXnXS SSSSS |
| 30031 | * SA * Sm5C * RG * SC * Sm5C * SmG * SmU * SmG * SmC * SmA | | SRSSS SSSS |
| WV- | m5Ceo * SAeon001m5Ceon001Aeon001Teo * SA * SG * SA * SG * SG | CACATAGAGGACGCCGUGCA | SnXnXnXS SSSSS |
| 30032 | * SA * Sm5C * SG * RC * Sm5C * SmG * SmU * SmG * SmC * SmA | | SSRSS SSSS |
| WV- | Geo * Sm5Ceon001Aeon001m5Ceon001Aeo * ST * SA * SG * SA * SG | GCACATAGAGGACGCCGUGC | SnXnXnXS SSSSS |
| 30033 | * SG * SA * Rm5C * SG * SC * Sm5mC * SmG * SmU * SmG * SmC | | SRSSS SSSS |
| WV- | Geo * Sm5Ceon001Aeon001m5Ceon001Aeo * ST * SA * SG * SA * SG | GCACATAGAGGACGCCGUGC | SnXnXnXS SSSSS |
| 30034 | * SG * SA * Sm5C * RG * SC * Sm5mC * SmG * SmU * SmG * SmC | | SSRSS SSSS |
| WV- | Geo * Sm5Ceon001Aeon001m5Ceon001Aeo * ST * SA * SG * SA * SG | GCACATAGAGGACGCCGUGC | SnXnXnXS SSSSS |
| 30035 | * SG * SA * Sm5C * SG * RC * Sm5mC * SmG * SmU * SmG * SmC | | SSSRS SSSS |
| WV- | mA * SmG * SmA * SmG * SmG * SA * Rm5C * SG * SC * Sm5C * | AGAGGACGCCGTGCAGGGCT | SSSSS RSSSS |
| 30036 | SG * ST * SG * SC * SA * SGeon001Geon001Geon001m5Ceo * STeo | | SSSSS nXnXnXS |
| WV- | mA * SmG * SmA * SmG * SmG * SA * 5m5C * RG * SC * 5m5C * | AGAGGACGCCGTGCAGGGCT | SSSSS SRSSS |
| 30037 | SG * ST * SG * SC * SA * SGeon001Geon001Geon001m5Ceo * STeo | | SSSSS nXnXnXS |
| WV- | mA * SmG * SmA * SmG * SmG * SA * Sm5C * SG * RC * Sm5C * | AGAGGACGCCGTGCAGGGCT | SSSSS SSRSS |
| 30038 | SG * ST * SG * SC * SA * SGeon001Geon001Geon001m5Ceo * STeo | | SSSSS nXnXnXS |
| WV- | mU * SmA * SmG * SmA * SmG * SG * SA * Rm5C * SG * SC * | UAGAGGACGCCGTGCAGGGC | SSSSS SRSSS |
| 30039 | 5m5C * SG * ST * SG * SC * SAeon001Geon001Geon001Geo * | | SSSSS nXnXnXS |
| | Sm5Ceo | | |
| WV- | mU * SmA * SmG * SmA * SmG * SG * SA * 5m5C * RG * SC * | UAGAGGACGCCGTGCAGGGC | SSSSS SSRSS |
| 30040 | 5m5C * SG * ST * SG * SC * SAeon001Geon001Geon001Geo * | | SSSSS nXnXnXS |
| | Sm5Ceo | | |
| WV- | mU * SmA * SmG * SmA * SmG * SG * SA * 5m5C * SG * RC * | UAGAGGACGCCGTGCAGGGC | SSSSS SSSRS |
| 30041 | 5m5C * SG * ST * SG * SC * SAeon001Geon001Geon001Geo * | | SSSSS nXnXnXS |
| | Sm5Ceo | | |
| WV- | mA * SmU * SmA * SmG * SmA * SG * SG * SA * Rm5C * SG * SC * | AUAGAGGACGCCGTGCAGGG | SSSSS SSRSS |
| 30042 | 5m5C * SG * ST * SG * Sm5Ceon001Aeon001Geon001Geo * SGeo | | SSSSS nXnXnXS |
| WV- | mA * SmU * SmA * SmG * SmA * SG * SG * SA * 5m5C * RG * SC * | AUAGAGGACGCCGTGCAGGG | SSSSS SSSRS |
| 30043 | 5m5C * SG * ST * SG * Sm5Ceon001Aeon001Geon001Geo * SGeo | | SSSSS nXnXnXS |
| WV- | mA * SmU * SmA * SmG * SmA * SG * SG * SA * 5m5C * SG * RC * | AUAGAGGACGCCGTGCAGGG | SSSSS SSSSR |
| 30044 | 5m5C * SG * ST * SG * Sm5Ceon001Aeon001Geon001Geo * SGeo | | SSSSS nXnXnXS |
| WV- | mC * SmA * SmU * SmA * SmG * SA * SG * SG * SA * Rm5C * SG * | CAUAGAGGACGCCGTGCAGG | SSSSS SSSRS |
| 30045 | SC * Sm5C * SG * ST * SGeon001m5Ceon001Aeon001Geo * SGeo | | SSSSS nXnXnXS |
| WV- | mC * SmA * SmU * SmA * SmG * SA * SG * SG * SA * Sm5C * RG * | CAUAGAGGACGCCGTGCAGG | SSSSS SSSSR |
| 30046 | SC * Sm5C * SG * ST * SGeon001m5Ceon001Aeon001Geo * SGeo | | SSSSS nXnXnXS |
| WV- | mC * SmA * SmU * SmA * SmG * SA * SG * SG * SA * Sm5C * SG * | CAUAGAGGACGCCGTGCAGG | SSSSS SSSSS |
| 30047 | RC * Sm5C * SG * ST * SGeon001m5Ceon001Aeon001Geo * SGeo | | RSSSS nXnXnXS |
| WV- | mA * SmC * SmA * SmU * SmA * SG * SA * SG * SG * SA * Rm5C * | ACAUAGAGGACGCCGTGCAG | SSSSS SSSSR |
| 30048 | SG * SC * Sm5C * SG * STeon001Geon001m5Ceon001Aeo * SGeo | | SSSSS nXnXnXS |
| WV- | mA * SmC * SmA * SmU * SmA * SG * SA * SG * SG * SA * Sm5C * | ACAUAGAGGACGCCGTGCAG | SSSSS SSSSS |
| 30049 | RG * SC * Sm5C * SG * STeon001Geon001m5Ceon001Aeo * SGeo | | RSSSS nXnXnXS |
| WV- | mA * SmC * SmA * SmU * SmA * SG * SA * SG * SG * SA * Sm5C * | ACAUAGAGGACGCCGTGCAG | SSSSS SSSSS |
| 30050 | SG * RC * Sm5C * SG * STeon001Geon001m5Ceon001Aeo * SGeo | | SRSSS nXnXnXS |
| WV- | mC * SmA * SmC * SmA * SmU * SA * SG * SA * SG * SG * SA * | CACAUAGAGGACGCCGTGCA | SSSSS SSSSS |
| 30051 | Rm5C * SG * SC * Sm5C * SGeon001Teon001Geon001m5Ceo * SAeo | | RSSSS nXnXnXS |
| WV- | mC * SmA * SmC * SmA * SmU * SA * SG * SA * SG * SG * SA * | CACAUAGAGGACGCCGTGCA | SSSSS SSSSS |
| 30052 | Sm5C * RG * SC * Sm5C * SGeon001Teon001Geon001m5Ceo * SAeo | | SRSSS nXnXnXS |
| WV- | mc * SmA * SmC * SmA * SmU * SA * SG * SA * SG * SG * SA * | CACAUAGAGGACGCCGTGCA | SSSSS SSSSS |
| 30053 | Sm5C * SG * RC * Sm5C * SGeon001Teon001Geon001m5Ceo * SAeo | | SSRSS nXnXnXS |
| WV- | mG * SmC * SmA * SmC * SmA * ST * SA * SG * SA * SG * SG * SA | GCACATAGAGGACGCCGTGC | SSSSS SSSSS |
| 30054 | * Rm5C * SG * SC * Sm5Ceon001Geon001Teon001Geo * Sm5Ceo | | SRSSS nXnXnXS |
| WV- | mG * SmC * SmA * SmC * SmA * ST * SA * SG * SA * SG * SG * SA | GCACATAGAGGACGCCGTGC | SSSSS SSSSS |
| 30055 | * Sm5C * RG * SC * Sm5Ceon001Geon001Teon001Geo * Sm5Ceo | | SSRSS nXnXnXS |
| WV- | mG * SmC * SmA * SmC * SmA * ST * SA * SG * SA * SG * SG * SA | GCACATAGAGGACGCCGTGC | SSSSS SSSSS |
| 30056 | * Sm5C * SG * RC * Sm5Ceon001Geon001Teon001Geo * 5m5Ceo | | SSSRS nXnXnXS |
| WV- | Teo * SGeoAeoGeom5Ceo * SG * RG * SA * SG * SA * SA * SA * | TGAGCGGAGAAACCCTCCAA | SOOOS RSSSS |
| 30057 | SC * SC * SC * STeom5Ceom5CeoAeo * SAeo | | SSSSS OOOS |
| WV- | Teo * SGeoAeoGeom5Ceo * SG * SG * RA * SG * SA * SA * SA * | TGAGCGGAGAAACCCTCCAA | SOOOS SRSSS |
| 30058 | SC * SC * SC * STeom5Ceom5CeoAeo * SAeo | | SSSSS OOOS |
| WV- | Teo * SGeoAeoGeom5Ceo * SG * SG * SA * RG * SA * SA * SA * | TGAGCGGAGAAACCCTCCAA | SOOOS SSRSS |
| 30059 | SC * SC * SC * STeom5Ceom5CeoAeo * SAeo | | SSSSS OOOS |
| WV- | m5Ceo * STeoGeoAeoGeo * Sm5C * SG * RG * SA * SG * SA * SA | CTGAGCGGAGAAACCCTCCA | SOOOS SRSSS |
| 30060 | * SA * SC * SC * Sm5CeoTeom5Ceom5Ceo * SAeo | | SSSSS OOOS |
| WV- | m5Ceo * STeoGeoAeoGeo * Sm5C * SG * SG * RA * SG * SA * SA | CTGAGCGGAGAAACCCTCCA | SOOOS SSRSS |
| 30061 | * SA * SC * SC * Sm5CeoTeom5Ceom5Ceo * SAeo | | SSSSS OOOS |
| WV- | m5Ceo * STeoGeoAeoGeo * Sm5C * SG * SG * SA * RG * SA * SA | CTGAGCGGAGAAACCCTCCA | SOOOS SSSRS |
| 30062 | * SA * SC * SC * Sm5CeoTeom5Ceom5Ceo * SAeo | | SSSSS OOOS |
| WV- | Geo * Sm5CeoTeoGeoAeo * SG * Sm5C * SG * RG * SA * SG * SA | GCTGAGCGGAGAAACCCTCC | SOOOS SSRSS |
| 30063 | * SA * SA * SC * Sm5Ceom5CeoTeom5Ceo * Sm5Ceo | | SSSSS OOOS |
| WV- | Geo * Sm5CeoTeoGeoAeo * SG * Sm5C * SG * SG * RA * SG * SA | GCTGAGCGGAGAAACCCTCC | SOOOS SSSRS |
| 30064 | * SA * SA * SC * Sm5Ceom5CeoTeom5Ceo * Sm5Ceo | | SSSSS OOOS |
| WV- | Geo * Sm5CeoTeoGeoAeo * SG * Sm5C * SG * SG * SA * RG * SA | GCTGAGCGGAGAAACCCTCC | SOOOS SSSSR |
| 30065 | * SA * SA * SC * Sm5Ceom5CeoTeom5Ceo * Sm5Ceo | | SSSSS OOOS |
| WV- | Geo * SGeom5CeoTeoGeo * SA * SG * Sm5C * SG * RG * SA * SG | GGCTGAGCGGAGAAACCCTC | SOOOS SSSRS |
| 30066 | * SA * SA * SA * Sm5Ceom5Ceom5CeoTeo * Sm5Ceo | | SSSSS OOOS |
| WV- | Geo * SGeom5CeoTeoGeo * SA * SG * Sm5C * SG * SG * RA * SG | GGCTGAGCGGAGAAACCCTC | SOOOS SSSSR |
| 30067 | * SA * SA * SA * Sm5Ceom5Ceom5CeoTeo * Sm5Ceo | | SSSSS OOOS |
| WV- | Geo * SGeom5CeoTeoGeo * SA * SG * Sm5C * SG * SG * SA * RG | GGCTGAGCGGAGAAACCCTC | SOOOS SSSSS |
| 30068 | * SA * SA * SA * Sm5Ceom5Ceom5CeoTeo * Sm5Ceo | | RSSSS OOOS |
| WV- | Aeo * SGeoGeom5CeoTeo * SG * SA * SG * Sm5C * SG * RG * SA | AGGCTGAGCGGAGAAACCCT | SOOOS SSSSR |
| 30069 | * SG * SA * SA * SAeom5Ceom5Ceom5Ceo * STeo | | SSSSS OOOS |
| WV- | Aeo * SGeoGeom5CeoTeo * SG * SA * SG * Sm5C * SG * SG * RA | AGGCTGAGCGGAGAAACCCT | SOOOS SSSSS |
| 30070 | * SG * SA * SA * SAeom5Ceom5Ceom5Ceo * STeo | | RSSSS OOOS |
| WV- | Aeo * SGeoGeom5CeoTeo * SG * SA * SG * Sm5C * SG * SG * SA | AGGCTGAGCGGAGAAACCCT | SOOOS SSSSS |
| 30071 | * RG * SA * SA * SAeom5Ceom5Ceom5Ceo * STeo | | SRSSS OOOS |
| WV- | Aeo * SAeoGeoGeom5Ceo * ST * SG * SA * SG * Sm5C * SG * RG | AAGGCTGAGCGGAGAAACCC | SOOOS SSSSS |
| 30072 | * SA * SG * SA * SAeoAeom5Ceom5Ceo * Sm5Ceo | | RSSSS OOOS |
| WV- | Aeo * SAeoGeoGeom5Ceo * ST * SG * SA * SG * Sm5C * SG * SG | AAGGCTGAGCGGAGAAACCC | SOOOS SSSSS |
| 30073 | * RA * SG * SA * SAeoAeom5Ceom5Ceo * Sm5Ceo | | SRSSS OOOS |
| WV- | Aeo * SAeoGeoGeom5Ceo * ST * SG * SA * SG * Sm5C * SG * SG | AAGGCTGAGCGGAGAAACCC | SOOOS SSSSS |
| 30074 | * SA * RG * SA * SAeoAeom5Ceom5Ceo * Sm5Ceo | | SSRSS OOOS |
| WV- | m5Ceo * SAeoAeoGeoGeo * SC * ST * SG * SA * SG * Sm5C * SG | CAAGGCTGAGCGGAGAAACC | SOOOS SSSSS |
| 30075 | * RG * SA * SG * SAeoAeoAeom5Ceo * Sm5Ceo | | SRSSS OOOS |
| WV- | m5Ceo * SAeoAeoGeoGeo * SC * ST * SG * SA * SG * Sm5C * SG | CAAGGCTGAGCGGAGAAACC | SOOOS SSSSS |
| 30076 | * SG * RA * SG * SAeoAeoAeom5Ceo * Sm5Ceo | | SSRSS OOOS |
| WV- | m5Ceo * SAeoAeoGeoGeo * SC * ST * SG * SA * SG * Sm5C * SG | CAAGGCTGAGCGGAGAAACC | SOOOS SSSSS |
| 30077 | * SG * SA * RG * SAeoAeoAeom5Ceo * Sm5Ceo | | SSSRS OOOS |
| WV- | Teo * SGeoAeoGeom5Ceo * SG * RG * SA * SG * SA * SA * SA * | TGAGCGGAGAAACCCUCCAA | SOOOS RSSSS |
| 30078 | SC * SC * SC * SmU * SmC * SmC * SmA * SmA | | SSSSS SSSS |
| WV- | Teo * SGeoAeoGeom5Ceo * SG * SG * RA * SG * SA * SA * SA * | TGAGCGGAGAAACCCUCCAA | SOOOS SRSSS |
| 30079 | SC * SC * SC * SmU * SmC * SmC * SmA * SmA | | SSSSS SSSS |
| WV- | Teo * SGeoAeoGeom5Ceo * SG * SG * SA * RG * SA * SA * SA * | TGAGCGGAGAAACCCUCCAA | SOOOS SSRSS |
| 30080 | SC * SC * SC * SmU * SmC * SmC * SmA * SmA | | SSSSS SSSS |
| WV- | m5Ceo * STeoGeoAeoGeo * Sm5C * SG * RG * SA * SG * SA * SA | CTGAGCGGAGAAACCCUCCA | SOOOS SRSSS |
| 30081 | * SA * SC * SC * SmC * SmU * SmC * SmC * SmA | | SSSSS SSSS |
| WV- | m5Ceo * STeoGeoAeoGeo * Sm5C * SG * SG * RA * SG * SA * SA | CTGAGCGGAGAAACCCUCCA | SOOOS SSRSS |
| 30082 | * SA * SC * SC * SmC * SmU * SmC * SmC * SmA | | SSSSS SSSS |
| WV- | m5Ceo * STeoGeoAeoGeo * Sm5C * SG * SG * SA * RG * SA * SA | CTGAGCGGAGAAACCCUCCA | SOOOS SSSRS |
| 30083 | * SA * SC * SC * SmC * SmU * SmC * SmC * SmA | | SSSSS SSSS |
| WV- | Geo * Sm5CeoTeoGeoAeo * SG * Sm5C * SG * RG * SA * SG * SA | GCTGAGCGGAGAAACCCUCC | SOOOS SSRSS |
| 30084 | * SA * SA * SC * SmC * SmC * SmU * SmC * SmC | | SSSSS SSSS |
| WV- | Geo * Sm5CeoTeoGeoAeo * SG * Sm5C * SG * SG * RA * SG * SA | GCTGAGCGGAGAAACCCUCC | SOOOS SSSRS |
| 30085 | * SA * SA * SC * SmC * SmC * SmU * SmC * SmC | | SSSSS SSSS |
| WV- | Geo * Sm5CeoTeoGeoAeo * SG * Sm5C * SG * SG * SA * RG * SA | GCTGAGCGGAGAAACCCUCC | SOOOS SSSSR |
| 30086 | * SA * SA * SC * SmC * SmC * SmU * SmC * SmC | | SSSSS SSSS |
| WV- | Geo * SGeom5CeoTeoGeo * SA * SG * Sm5C * SG * RG * SA * SG | GGCTGAGCGGAGAAACCCUC | SOOOS SSSRS |
| 30087 | * SA * SA * SA * SmC * SmC * SmC * SmU * SmC | | SSSSS SSSS |
| WV- | Geo * SGeom5CeoTeoGeo * SA * SG * Sm5C * SG * SG * RA * SG | GGCTGAGCGGAGAAACCCUC | SOOOS SSSSR |
| 30088 | * SA * SA * SA * SmC * SmC * SmC * SmU * SmC | | SSSSS SSSS |
| WV- | Geo * SGeom5CeoTeoGeo * SA * SG * Sm5C * SG * SG * SA * RG | GGCTGAGCGGAGAAACCCUC | SOOOS SSSSS |
| 30089 | * SA * SA * SA * SmC * SmC * SmC * SmU * SmC | | RSSSS SSSS |
| WV- | Aeo * SGeoGeom5CeoTeo * SG * SA * SG * Sm5C * SG * RG * SA | AGGCTGAGCGGAGAAACCCU | SOOOS SSSSR |
| 30090 | * SG * SA * SA * SmA * SmC * SmC * SmC * SmU | | SSSSS SSSS |
| WV- | Aeo * SGeoGeom5CeoTeo * SG * SA * SG * Sm5C * SG * SG * RA | AGGCTGAGCGGAGAAACCCU | SOOOS SSSSS |
| 30091 | * SG * SA * SA * SmA * SmC * SmC * SmC * SmU | | RSSSS SSSS |
| WV- | Aeo * SGeoGeom5CeoTeo * SG * SA * SG * Sm5C * SG * SG * SA | AGGCTGAGCGGAGAAACCCU | SOOOS SSSSS |
| 30092 | * RG * SA * SA * SmA * SmC * SmC * SmC * SmU | | SRSSS SSSS |
| WV- | Aeo * SAeoGeoGeom5Ceo * ST * SG * SA * SG * Sm5C * SG * RG | AAGGCTGAGCGGAGAAACCC | SOOOS SSSSS |
| 30093 | * SA * SG * SA * SmA * SmA * SmC * SmC * SmC | | RSSSS SSSS |
| WV- | Aeo * SAeoGeoGeom5Ceo * ST * SG * SA * SG * Sm5C * SG * SG | AAGGCTGAGCGGAGAAACCC | SOOOS SSSSS |
| 30094 | * RA * SG * SA * SmA * SmA * SmC * SmC * SmC | | SRSSS SSSS |
| WV- | Aeo * SAeoGeoGeom5Ceo * ST * SG * SA * SG * Sm5C * SG * SG | AAGGCTGAGCGGAGAAACCC | SOOOS SSSSS |
| 30095 | * SA * RG * SA * SmA * SmA * SmC * SmC * SmC | | SSRSS SSSS |
| WV- | m5Ceo * SAeoAeoGeoGeo * SC * ST * SG * SA * SG * Sm5C * SG | CAAGGCTGAGCGGAGAAACC | SOOOS SSSSS |
| 30096 | * RG * SA * SG * SmA * SmA * SmA * SmC * SmC | | SRSSS SSSS |
| WV- | m5Ceo * SAeoAeoGeoGeo * SC * ST * SG * SA * SG * Sm5C * SG | CAAGGCTGAGCGGAGAAACC | SOOOS SSSSS |
| 30097 | * SG * RA * SG * SmA * SmA * SmA * SmC * SmC | | SSRSS SSSS |
| WV- | m5Ceo * SAeoAeoGeoGeo * SC * ST * SG * SA * SG * Sm5C * SG | CAAGGCTGAGCGGAGAAACC | SOOOS SSSSS |
| 30098 | * SG * SA * RG * SmA * SmA * SmA * SmC * SmC | | SSSRS SSSS |
| WV- | mU * SmG * SmA * SmG * Sm5mC * SG * RG * SA * SG * SA * | UGAGCGGAGAAACCCTCCAA | SSSSS RSSSS |
| 30099 | SA * SA * SC * SC * SC * STeom5Ceom5CeoAeo * SAeo | | SSSSS OOOS |
| WV- | mU * SmG * SmA * SmG * Sm5mC * SG * SG * RA * SG * SA * | UGAGCGGAGAAACCCTCCAA | SSSSS SRSSS |
| 30100 | SA * SA * SC * SC * SC * STeom5Ceom5CeoAeo * SAeo | | SSSSS OOOS |
| WV- | mU * SmG * SmA * SmG * Sm5mC * SG * SG * SA * RG * SA * | UGAGCGGAGAAACCCTCCAA | SSSSS SSRSS |
| 30101 | SA * SA * SC * SC * SC * STeom5Ceom5CeoAeo * SAeo | | SSSSS OOOS |
| WV- | mC * SmU * SmG * SmA * SmG * Sm5C * SG * RG * SA * SG * SA | CUGAGCGGAGAAACCCTCCA | SSSSS SRSSS |
| 30102 | * SA * SA * SC * SC * Sm5CeoTeom5Ceom5Ceo * SAeo | | SSSSS OOOS |
| WV- | mC * SmU * SmG * SmA * SmG * Sm5C * SG * SG * RA * SG * SA | CUGAGCGGAGAAACCCTCCA | SSSSS SSRSS |
| 30103 | * SA * SA * SC * SC * Sm5CeoTeom5Ceom5Ceo * SAeo | | SSSSS OOOS |
| WV- | mC * SmU * SmG * SmA * SmG * Sm5C * SG * SG * SA * RG * SA | CUGAGCGGAGAAACCCTCCA | SSSSS SSSRS |
| 30104 | * SA * SA * SC * SC * Sm5CeoTeom5Ceom5Ceo * SAeo | | SSSSS OOOS |
| WV- | mG * SmC * SmU * SmG * SmA * SG * Sm5C * SG * RG * SA * SG | GCUGAGCGGAGAAACCCTCC | SSSSS SSRSS |
| 30105 | * SA * SA * SA * SC * Sm5Ceom5CeoTeom5Ceo * Sm5Ceo | | SSSSS OOOS |
| WV- | mG * SmC * SmU * SmG * SmA * SG * Sm5C * SG * SG * RA * SG | GCUGAGCGGAGAAACCCTCC | SSSSS SSSRS |
| 30106 | * SA * SA * SA * SC * Sm5Ceom5CeoTeom5Ceo * Sm5Ceo | | SSSSS OOOS |
| WV- | mG * SmC * SmU * SmG * SmA * SG * Sm5C * SG * SG * SA * RG | GCUGAGCGGAGAAACCCTCC | SSSSS SSSSR |
| 30107 | * SA * SA * SA * SC * Sm5Ceom5CeoTeom5Ceo * Sm5Ceo | | SSSSS OOOS |
| WV- | mG * SmG * SmC * SmU * SmG * SA * SG * Sm5C * SG * RG * SA | GGCUGAGCGGAGAAACCCTC | SSSSS SSSRS |
| 30108 | * SG * SA * SA * SA * Sm5Ceom5Ceom5CeoTeo * Sm5Ceo | | SSSSS OOOS |
| WV- | mG * SmG * SmC * SmU * SmG * SA * SG * Sm5C * SG * SG * RA | GGCUGAGCGGAGAAACCCTC | SSSSS SSSSR |
| 30109 | * SG * SA * SA * SA * Sm5Ceom5Ceom5CeoTeo * Sm5Ceo | | SSSSS OOOS |
| WV- | mG * SmG * SmC * SmU * SmG * SA * SG * Sm5C * SG * SG * SA | GGCUGAGCGGAGAAACCCTC | SSSSS SSSSS |
| 30110 | * RG * SA * SA * SA * Sm5Ceom5Ceom5CeoTeo * Sm5Ceo | | RSSSS OOOS |
| WV- | mA * SmG * SmG * SmC * SmU * SG * SA * SG * Sm5C * SG * RG | AGGCUGAGCGGAGAAACCCT | SSSSS SSSSR |
| 30111 | * SA * SG * SA * SA * SAeom5Ceom5Ceom5Ceo * STeo | | SSSSS OOOS |
| WV- | mA * SmG * SmG * SmC * SmU * SG * SA * SG * Sm5C * SG * SG | AGGCUGAGCGGAGAAACCCT | SSSSS SSSSS |
| 30112 | * RA * SG * SA * SA * SAeom5Ceom5Ceom5Ceo * STeo | | RSSSS OOOS |
| WV- | mA * SmG * SmG * SmC * SmU * SG * SA * SG * Sm5C * SG * SG | AGGCUGAGCGGAGAAACCCT | SSSSS SSSSS |
| 30113 | * SA * RG * SA * SA * SAeom5Ceom5Ceom5Ceo * STeo | | SRSSS OOOS |
| WV- | mA * SmA * SmG * SmG * SmC * ST * SG * SA * SG * Sm5C * SG | AAGGCTGAGCGGAGAAACCC | SSSSS SSSSS |
| 30114 | * RG * SA * SG * SA * SAeoAeom5Ceom5Ceo * Sm5Ceo | | RSSSS OOOS |
| WV- | mA * SmA * SmG * SmG * SmC * ST * SG * SA * SG * Sm5C * SG | AAGGCTGAGCGGAGAAACCC | SSSSS SSSSS |
| 30115 | * SG * RA * SG * SA * SAeoAeom5Ceom5Ceo * Sm5Ceo | | SRSSS OOOS |
| WV- | mA * SmA * SmG * SmG * SmC * ST * SG * SA * SG * Sm5C * SG | AAGGCTGAGCGGAGAAACCC | SSSSS SSSSS |
| 30116 | * SG * SA * RG * SA * SAeoAeom5Ceom5Ceo * Sm5Ceo | | SSRSS OOOS |
| WV- | mC * SmA * SmA * SmG * SmG * SC * ST * SG * SA * SG * Sm5C | CAAGGCTGAGCGGAGAAACC | SSSSS SSSSS |
| 30117 | * SG * RG * SA * SG * SAeoAeoAeom5Ceo * Sm5Ceo | | SRSSS OOOS |
| WV- | mC * SmA * SmA * SmG * SmG * SC * ST * SG * SA * SG * Sm5C | CAAGGCTGAGCGGAGAAACC | SSSSS SSSSS |
| 30118 | * SG * SG * RA * SG * SAeoAeoAeom5Ceo * Sm5Ceo | | SSRSS OOOS |
| WV- | mC * SmA * SmA * SmG * SmG * SC * ST * SG * SA * SG * Sm5C | CAAGGCTGAGCGGAGAAACC | SSSSS SSSSS |
| 30119 | * SG * SG * SA * RG * SAeoAeoAeom5Ceo * Sm5Ceo | | SSSRS OOOS |
| WV- | Teo * SGeon001Aeon001Geon001m5Ceo * SG * RG * SA * SG * SA | TGAGCGGAGAAACCCTCCAA | SnXnXnXS RSSSS |
| 30120 | * SA * SA * SC * SC * SC * STeom5Ceom5CeoAeo * SAeo | | SSSSS OOOS |
| WV- | Teo * SGeon001Aeon001Geon001m5Ceo * SG * SG * RA * SG * SA | TGAGCGGAGAAACCCTCCAA | SnXnXnXS SRSSS |
| 30121 | * SA * SA * SC * SC * SC * STeom5Ceom5CeoAeo * SAeo | | SSSSS OOOS |
| WV- | Teo * SGeon001Aeon001Geon001m5Ceo * SG * SG * SA * RG * SA | TGAGCGGAGAAACCCTCCAA | SnXnXnXS SSRSS |
| 30122 | * SA * SA * SC * SC * SC * STeom5Ceom5CeoAeo * SAeo | | SSSSS OOOS |
| WV- | m5Ceo * STeon001Geon001Aeon001Geo * Sm5C * SG * RG * SA * | CTGAGCGGAGAAACCCTCCA | SnXnXnXS SRSSS |
| 30123 | SG * SA * SA * SA * SC * SC * Sm5CeoTeom5Ceom5Ceo * SAeo | | SSSSS OOOS |
| WV- | m5Ceo * STeon001Geon001Aeon001Geo * 5m5C * SG * SG * RA * | CTGAGCGGAGAAACCCTCCA | SnXnXnXS SSRSS |
| 30124 | SG * SA * SA * SA * SC * SC * Sm5CeoTeom5Ceom5Ceo * SAeo | | SSSSS OOOS |
| WV- | m5Ceo * STeon001Geon001Aeon001Geo * 5m5C * SG * SG * SA * | CTGAGCGGAGAAACCCTCCA | SnXnXnXS SSSRS |
| 30125 | RG * SA * SA * SA * SC * SC * Sm5CeoTeom5Ceom5Ceo * SAeo | | SSSSS OOOS |
| WV- | Geo * Sm5Ceon001Teon001Geon001Aeo * SG * Sm5C * SG * RG * | GCTGAGCGGAGAAACCCTCC | SnXnXnXS SSRSS |
| 30126 | SA * SG * SA * SA * SA * SC * Sm5Ceom5CeoTeom5Ceo * Sm5Ceo | | SSSSS OOOS |
| WV- | Geo * Sm5Ceon001Teon001Geon001Aeo * SG * Sm5C * SG * SG * | GCTGAGCGGAGAAACCCTCC | SnXnXnXS SSSRS |
| 30127 | RA * SG * SA * SA * SA * SC * Sm5Ceom5CeoTeom5Ceo * Sm5Ceo | | SSSSS OOOS |
| WV- | Geo * Sm5Ceon001Teon001Geon001Aeo * SG * Sm5C * SG * SG * | GCTGAGCGGAGAAACCCTCC | SnXnXnXS SSSSR |
| 30128 | SA * RG * SA * SA * SA * SC * Sm5Ceom5CeoTeom5Ceo * Sm5Ceo | | SSSSS OOOS |
| WV- | Geo * SGeon001m5Ceon001Teon001Geo * SA * SG * Sm5C * SG * | GGCTGAGCGGAGAAACCCTC | SnXnXnXS SSSRS |
| 30129 | RG * SA * SG * SA * SA * SA * Sm5Ceom5Ceom5CeoTeo * Sm5Ceo | | SSSSS OOOS |
| WV- | Geo * SGeon001m5Ceon001Teon001Geo * SA * SG * Sm5C * SG * | GGCTGAGCGGAGAAACCCTC | SnXnXnXS SSSSR |
| 30130 | SG * RA * SG * SA * SA * SA * Sm5Ceom5Ceom5CeoTeo * Sm5Ceo | | SSSSS OOOS |
| WV- | Geo * SGeon001m5Ceon001Teon001Geo * SA * SG * Sm5C * SG * | GGCTGAGCGGAGAAACCCTC | SnXnXnXS SSSSS |
| 30131 | SG * SA * RG * SA * SA * SA * Sm5Ceom5Ceom5CeoTeo * Sm5Ceo | | RSSSS OOOS |
| WV- | Aeo * SGeon001Geon001m5Ceon001Teo * SG * SA * SG * Sm5C * | AGGCTGAGCGGAGAAACCCT | SnXnXnXS SSSSR |
| 30132 | SG * RG * SA * SG * SA * SA * SAeom5Ceom5Ceom5Ceo * STeo | | SSSSS OOOS |
| WV- | Aeo * SGeon001Geon001m5Ceon001Teo * SG * SA * SG * Sm5C * | AGGCTGAGCGGAGAAACCCT | SnXnXnXS SSSSS |
| 30133 | SG * SG * RA * SG * SA * SA * SAeom5Ceom5Ceom5Ceo * STeo | | RSSSS OOOS |
| WV- | Aeo * SGeon001Geon001m5Ceon001Teo * SG * SA * SG * Sm5C * | AGGCTGAGCGGAGAAACCCT | SnXnXnXS SSSSS |
| 30134 | SG * SG * SA * RG * SA * SA * SAeom5Ceom5Ceom5Ceo * STeo | | SRSSS OOOS |
| WV- | Aeo * SAeon001Geon001Geon001m5Ceo * ST * SG * SA * SG * | AAGGCTGAGCGGAGAAACCC | SnXnXnXS SSSSS |
| 30135 | Sm5C * SG * RG * SA * SG * SA * SAeoAeom5Ceom5Ceo * | | RSSSS OOOS |
| | Sm5Ceo | | |
| WV- | Aeo * SAeon001Geon001Geon001m5Ceo * ST * SG * SA * SG * | AAGGCTGAGCGGAGAAACCC | SnXnXnXS SSSSS |
| 30136 | Sm5C * SG * SG * RA * SG * SA * SAeoAeom5Ceom5Ceo * | | SRSSS OOOS |
| | Sm5Ceo | | |
| WV- | Aeo * SAeon001Geon001Geon001m5Ceo * ST * SG * SA * SG * | AAGGCTGAGCGGAGAAACCC | SnXnXnXS SSSSS |
| 30137 | Sm5C * SG * SG * SA * RG * SA * SAeoAeom5Ceom5Ceo * | | SSRSS OOOS |
| | Sm5Ceo | | |
| WV- | m5Ceo * SAeon001Aeon001Geon001Geo * SC * ST * SG * SA * SG | CAAGGCTGAGCGGAGAAACC | SnXnXnXS SSSSS |
| 30138 | * Sm5C * SG * RG * SA * SG * SAeoAeoAeom5Ceo * Sm5Ceo | | SRSSS OOOS |
| WV- | m5Ceo * SAeon001Aeon001Geon001Geo * SC * ST * SG * SA * SG | CAAGGCTGAGCGGAGAAACC | SnXnXnXS SSSSS |
| 30139 | * Sm5C * SG * SG * RA * SG * SAeoAeoAeom5Ceo * Sm5Ceo | | SSRSS OOOS |
| WV- | m5Ceo * SAeon001Aeon001Geon001Geo * SC * ST * SG * SA * SG | CAAGGCTGAGCGGAGAAACC | SnXnXnXS SSSSS |
| 30140 | * 5m5C * SG * SG * SA * RG * SAeoAeoAeom5Ceo * 5m5Ceo | | SSSRS OOOS |
| WV- | Teo * SGeoAeoGeom5Ceo * SG * RG * SA * SG * SA * SA * SA * | TGAGCGGAGAAACCCTCCAA | SOOOS RSSSS |
| 30141 | SC * SC * SC * STeon001m5Ceon001m5Ceon001Aeo * SAeo | | SSSSS nXnXnXS |
| WV- | Teo * SGeoAeoGeom5Ceo * SG * SG * RA * SG * SA * SA * SA * | TGAGCGGAGAAACCCTCCAA | SOOOS SRSSS |
| 30142 | SC * SC * SC * STeon001m5Ceon001m5Ceon001Aeo * SAeo | | SSSSS nXnXnXS |
| WV- | Teo * SGeoAeoGeom5Ceo * SG * SG * SA * RG * SA * SA * SA * | TGAGCGGAGAAACCCTCCAA | SOOOS SSRSS |
| 30143 | SC * SC * SC * STeon001m5Ceon001m5Ceon001Aeo * SAeo | | SSSSS nXnXnXS |
| WV- | m5Ceo * STeoGeoAeoGeo * Sm5C * SG * RG * SA * SG * SA * SA * | CTGAGCGGAGAAACCCTCCA | SOOOS SRSSS |
| 30144 | SA * SC * SC * Sm5Ceon001Teon001m5Ceon001m5Ceo * SAeo | | SSSSS nXnXnXS |
| WV- | m5Ceo * STeoGeoAeoGeo * Sm5C * SG * SG * RA * SG * SA * SA * | CTGAGCGGAGAAACCCTCCA | SOOOS SSRSS |
| 30145 | SA * SC * SC * Sm5Ceon001Teon001m5Ceon001m5Ceo * SAeo | | SSSSS nXnXnXS |
| WV- | m5Ceo * STeoGeoAeoGeo * Sm5C * SG * SG * SA * RG * SA * SA * | CTGAGCGGAGAAACCCTCCA | SOOOS SSSRS |
| 30146 | SA * SC * SC * Sm5Ceon001Teon001m5Ceon001m5Ceo * SAeo | | SSSSS nXnXnXS |
| WV- | Geo * Sm5CeoTeoGeoAeo * SG * Sm5C * SG * RG * SA * SG * SA * | GCTGAGCGGAGAAACCCTCC | SOOOS SSRSS |
| 30147 | SA * SA * SC * Sm5Ceon001m5Ceon001Teon001m5Ceo * Sm5Ceo | | SSSSS nXnXnXS |
| WV- | Geo * Sm5CeoTeoGeoAeo * SG * Sm5C * SG * SG * RA * SG * SA * | GCTGAGCGGAGAAACCCTCC | SOOOS SSSRS |
| 30148 | SA * SA * SC * Sm5Ceon001m5Ceon001Teon001m5Ceo * Sm5Ceo | | SSSSS nXnXnXS |
| WV- | Geo * Sm5CeoTeoGeoAeo * SG * Sm5C * SG * SG * SA * RG * SA * | GCTGAGCGGAGAAACCCTCC | SOOOS SSSSR |
| 30149 | SA * SA * SC * Sm5Ceon001m5Ceon001Teon001m5Ceo * Sm5Ceo | | SSSSS nXnXnXS |
| WV- | Geo * SGeom5CeoTeoGeo * SA * SG * Sm5C * SG * RG * SA * SG * | GGCTGAGCGGAGAAACCCTC | SOOOS SSSRS |
| 30150 | SA * SA * SA * Sm5Ceon001m5Ceon001m5Ceon001Teo * Sm5Ceo | | SSSSS nXnXnXS |
| WV- | Geo * SGeom5CeoTeoGeo * SA * SG * Sm5C * SG * SG * RA * SG * | GGCTGAGCGGAGAAACCCTC | SOOOS SSSSR |
| 30151 | SA * SA * SA * Sm5Ceon001m5Ceon001m5Ceon001Teo * Sm5Ceo | | SSSSS nXnXnXS |
| WV- | Geo * SGeom5CeoTeoGeo * SA * SG * Sm5C * SG * SG * SA * RG * | GGCTGAGCGGAGAAACCCTC | SOOOS SSSSS |
| 30152 | SA * SA * SA * Sm5Ceon001m5Ceon001m5Ceon001Teo * Sm5Ceo | | RSSSS nXnXnXS |
| WV- | Aeo * SGeoGeom5CeoTeo * SG * SA * SG * Sm5C * SG * RG * SA | AGGCTGAGCGGAGAAACCCT | SOOOS SSSSR |
| 30153 | * SG * SA * SA * SAeon001m5Ceon001m5Ceon001m5Ceo * STeo | | SSSSS nXnXnXS |
| WV- | Aeo * SGeoGeom5CeoTeo * SG * SA * SG * Sm5C * SG * SG * RA | AGGCTGAGCGGAGAAACCCT | SOOOS SSSSS |
| 30154 | * SG * SA * SA * SAeon001m5Ceon001m5Ceon001m5Ceo * STeo | | RSSSS nXnXnXS |
| WV- | Aeo * SGeoGeom5CeoTeo * SG * SA * SG * Sm5C * SG * SG * SA | AGGCTGAGCGGAGAAACCCT | SOOOS SSSSS |
| 30155 | * RG * SA * SA * SAeon001m5Ceon001m5Ceon001m5Ceo * STeo | | SRSSS nXnXnXS |
| WV- | Aeo * SAeoGeoGeom5Ceo * ST * SG * SA * SG * Sm5C * SG * RG | AAGGCTGAGCGGAGAAACCC | SOOOS SSSSS |
| 30156 | * SA * SG * SA * SAeon001Aeon001m5Ceon001m5Ceo * Sm5Ceo | | RSSSS nXnXnXS |
| WV- | Aeo * SAeoGeoGeom5Ceo * ST * SG * SA * SG * Sm5C * SG * SG | AAGGCTGAGCGGAGAAACCC | SOOOS SSSSS |
| 30157 | * RA * SG * SA * SAeon001Aeon001m5Ceon001m5Ceo * Sm5Ceo | | SRSSS nXnXnXS |
| WV- | Aeo * SAeoGeoGeom5Ceo * ST * SG * SA * SG * Sm5C * SG * SG | AAGGCTGAGCGGAGAAACCC | SOOOS SSSSS |
| 30158 | * SA * RG * SA * SAeon001Aeon001m5Ceon001m5Ceo * Sm5Ceo | | SSRSS nXnXnXS |
| WV- | m5Ceo * SAeoAeoGeoGeo * SC * ST * SG * SA * SG * Sm5C * SG | CAAGGCTGAGCGGAGAAACC | SOOOS SSSSS |
| 30159 | * RG * SA * SG * SAeon001Aeon001Aeon001m5Ceo * Sm5Ceo | | SRSSS nXnXnXS |
| WV- | m5Ceo * SAeoAeoGeoGeo * SC * ST * SG * SA * SG * Sm5C * SG | CAAGGCTGAGCGGAGAAACC | SOOOS SSSSS |
| 30160 | * SG * RA * SG * SAeon001Aeon001Aeon001m5Ceo * Sm5Ceo | | SSRSS nXnXnXS |
| WV- | m5Ceo * SAeoAeoGeoGeo * SC * ST * SG * SA * SG * Sm5C * SG | CAAGGCTGAGCGGAGAAACC | SOOOS SSSSS |
| 30161 | * SG * SA * RG * SAeon001Aeon001Aeon001m5Ceo * Sm5Ceo | | SSSRS nXnXnXS |
| WV- | Teo * SGeon001Aeon001Geon001m5Ceo * SG * RG * SA * SG * SA | TGAGCGGAGAAACCCUCCAA | SnXnXnXS RSSSS |
| 30162 | * SA * SA * SC * SC * SC * SmU * SmC * SmC * SmA * SmA | | SSSSS SSSS |
| WV- | Teo * SGeon001Aeon001Geon001m5Ceo * SG * SG * RA * SG * SA | TGAGCGGAGAAACCCUCCAA | SnXnXnXS SRSSS |
| 30163 | * SA * SA * SC * SC * SC * SmU * SmC * SmC * SmA * SmA | | SSSSS SSSS |
| WV- | Teo * SGeon001Aeon001Geon001m5Ceo * SG * SG * SA * RG * SA | TGAGCGGAGAAACCCUCCAA | SnXnXnXS SSRSS |
| 30164 | * SA * SA * SC * SC * SC * SmU * SmC * SmC SmA * SmA | | SSSSS SSSS |
| WV- | m5Ceo * STeon001Geon001Aeon001Geo * Sm5C * SG * RG * SA * | CTGAGCGGAGAAACCCUCCA | SnXnXnXS SRSSS |
| 30165 | SG * SA * SA * SA * SC * SC * SmC * SmU * SmC * SmC * SmA | | SSSSS SSSS |
| WV- | m5Ceo * STeon001Geon001Aeon001Geo * Sm5C * SG * SG * RA * | CTGAGCGGAGAAACCCUCCA | SnXnXnXS SSRSS |
| 30166 | SG * SA * SA * SA * SC * SC * SmC * SmU * SmC * SmC * SmA | | SSSSS SSSS |
| WV- | m5Ceo * STeon001Geon001Aeon001Geo * Sm5C * SG * SG * SA * | CTGAGCGGAGAAACCCUCCA | SnXnXnXS SSSRS |
| 30167 | RG * SA * SA * SA * SC * SC * SmC * SmU * SmC * SmC * SmA | | SSSSS SSSS |
| WV- | Geo * Sm5Ceon001Teon001Geon001Aeo * SG * Sm5C * SG * RG * | GCTGAGCGGAGAAACCCUCC | SnXnXnXS SSRSS |
| 30168 | SA * SG * SA * SA * SA * SC * SmC * SmC * SmU * SmC * SmC | | SSSSS SSSS |
| WV- | Geo * Sm5Ceon001Teon001Geon001Aeo * SG * Sm5C * SG * SG * | GCTGAGCGGAGAAACCCUCC | SnXnXnXS SSSRS |
| 30169 | RA * SG * SA * SA * SA * SC * SmC * SmC * SmU * SmC * SmC | | SSSSS SSSS |
| WV- | Geo * Sm5Ceon001Teon001Geon001Aeo * SG * Sm5C * SG * SG * | GCTGAGCGGAGAAACCCUCC | SnXnXnXS SSSSR |
| 30170 | SA * RG * SA * SA * SA * SC * SmC * SmC * SmU * SmC * SmC | | SSSSS SSSS |
| WV- | Geo * SGeon001m5Ceon001Teon001Geo * SA * SG * Sm5C * SG * | GGCTGAGCGGAGAAACCCUC | SnXnXnXS SSSRS |
| 30171 | RG * SA * SG * SA * SA * SA * SmC * SmC * SmC * SmU * SmC | | SSSSS SSSS |
| WV- | Geo * SGeon001m5Ceon001Teon001Geo * SA * SG * Sm5C * SG * | GGCTGAGCGGAGAAACCCUC | SnXnXnXS SSSSR |
| 30172 | SG * RA * SG * SA * SA * SA * SmC * SmC * SmC * SmU * SmC | | SSSSS SSSS |
| WV- | Geo * SGeon001m5Ceon001Teon001Geo * SA * SG * Sm5C * SG * | GGCTGAGCGGAGAAACCCUC | SnXnXnXS SSSSS |
| 30173 | SG * SA * RG * SA * SA * SA * SmC * SmC * SmC * SmU * SmC | | RSSSS SSSS |
| WV- | Aeo * SGeon001Geon001m5Ceon001Teo * SG * SA * SG * Sm5C * | AGGCTGAGCGGAGAAACCCU | SnXnXnXS SSSSR |
| 30174 | SG * RG * SA * SG * SA * SA * SmA * SmC * SmC * SmC * SmU | | SSSSS SSSS |
| WV- | Aeo * SGeon001Geon001m5Ceon001Teo * SG * SA * SG * Sm5C * | AGGCTGAGCGGAGAAACCCU | SnXnXnXS SSSSS |
| 30175 | SG * SG * RA * SG * SA * SA * SmA * SmC * SmC * SmC * SmU | | RSSSS SSSS |
| WV- | Aeo * SGeon001Geon001m5Ceon001Teo * SG * SA * SG * Sm5C * | AGGCTGAGCGGAGAAACCCU | SnXnXnXS SSSSS |
| 30176 | SG * SG * SA * RG * SA * SA * SmA * SmC * SmC * SmC * SmU | | SRSSS SSSS |
| WV- | Aeo * SAeon001Geon001Geon001m5Ceo * ST * SG * SA * SG * Sm5C | AAGGCTGAGCGGAGAAACCC | SnXnXnXS SSSSS |
| 30177 | * SG * RG * SA * SG * SA * SmA * SmA * SmC * SmC * SmC | | RSSSS SSSS |
| WV- | Aeo * SAeon001Geon001Geon001m5Ceo * ST * SG * SA * SG * Sm5C | AAGGCTGAGCGGAGAAACCC | SnXnXnXS SSSSS |
| 30178 | * SG * SG * RA * SG * SA * SmA * SmA * SmC * SmC * SmC | | SRSSS SSSS |
| WV- | Aeo * SAeon001Geon001Geon001m5Ceo * ST * SG * SA * SG * Sm5C | AAGGCTGAGCGGAGAAACCC | SnXnXnXS SSSSS |
| 30179 | * SG * SG * SA * RG * SA * SmA * SmA * SmC * SmC * SmC | | SSRSS SSSS |
| WV- | m5Ceo * SAeon001Aeon001Geon001Geo * SC * ST * SG * SA * SG * | CAAGGCTGAGCGGAGAAACC | SnXnXnXS SSSSS |
| 30180 | Sm5C * SG * RG * SA * SG * SmA * SmA * SmA * SmC * SmC | | SRSSS SSSS |
| WV- | m5Ceo * SAeon001Aeon001Geon001Geo * SC * ST * SG * SA * SG * | CAAGGCTGAGCGGAGAAACC | SnXnXnXS SSSSS |
| 30181 | Sm5C * SG * SG * RA * SG * SmA * SmA * SmA * SmC * SmC | | SSRSS SSSS |
| WV- | m5Ceo * SAeon001Aeon001Geon001Geo * SC * ST * SG * SA * SG * | CAAGGCTGAGCGGAGAAACC | SnXnXnXS SSSSS |
| 30182 | Sm5C * SG * SG * SA * RG * SmA * SmA * SmA * SmC * SmC | | SSSRS SSSS |
| WV- | mU * SmG * SmA * SmG * Sm5mC * SG * RG * SA * SG * SA * SA * | UGAGCGGAGAAACCCTCCAA | SSSSS RSSSS |
| 30183 | SA * SC * SC * SC * STeon001m5Ceon001m5Ceon001Aeo * SAeo | | SSSSS nXnXnXS |
| WV- | mU * SmG * SmA * SmG * Sm5mC * SG * SG * RA * SG * SA * SA * | UGAGCGGAGAAACCCTCCAA | SSSSS SRSSS |
| 30184 | SA * SC * SC * SC * STeon001m5Ceon001m5Ceon001Aeo * SAeo | | SSSSS nXnXnXS |
| WV- | mU * SmG * SmA * SmG * Sm5mC * SG * SG * SA * RG * SA * SA * | UGAGCGGAGAAACCCTCCAA | SSSSS SSRSS |
| 30185 | SA * SC * SC * SC * STeon001m5Ceon001m5Ceon001Aeo * SAeo | | SSSSS nXnXnXS |
| WV- | mC * SmU * SmG * SmA * SmG * Sm5C * SG * RG * SA * SG * SA * | CUGAGCGGAGAAACCCTCCA | SSSSS SRSSS |
| 30186 | SA * SA * SC * SC * Sm5Ceon001Teon001m5Ceon001m5Ceo * SAeo | | SSSSS nXnXnXS |
| WV- | mC * SmU * SmG * SmA * SmG * Sm5C * SG * SG * RA * SG * SA * | CUGAGCGGAGAAAC CCTCCA | SSSSS SSRSS |
| 30187 | SA * SA * SC * SC * Sm5Ceon001Teon001m5Ceon001m5Ceo * SAeo | | SSSSS nXnXnXS |
| WV- | mC * SmU * SmG * SmA * SmG * Sm5C * SG * SG * SA * RG * SA * | CUGAGCGGAGAAACCCTCCA | SSSSS SSSRS |
| 30188 | SA * SA * SC * SC * Sm5Ceon001Teon001m5Ceon001m5Ceo * SAeo | | SSSSS nXnXnXS |
| WV- | mG * SmC * SmU * SmG * SmA * SG * Sm5C * SG * RG * SA * SG * | GCUGAGCGGAGAAACCCTCC | SSSSS SSRSS |
| 30189 | SA * SA * SA * SC * Sm5Ceon001m5Ceon001Teon001m5Ceo * | | SSSSS nXnXnXS |
| | Sm5Ceo | | |
| WV- | mG * SmC * SmU * SmG * SmA * SG * Sm5C * SG * SG * RA * SG * | GCUGAGCGGAGAAACCCTCC | SSSSS SSSRS |
| 30190 | SA * SA * SA * SC * Sm5Ceon001m5Ceon001Teon001m5Ceo * | | SSSSS nXnXnXS |
| | Sm5Ceo | | |
| WV- | mG * SmC * SmU * SmG * SmA * SG * Sm5C * SG * SG * SA * RG * | GCUGAGCGGAGAAACCCTCC | SSSSS SSSSR |
| 30191 | SA * SA * SA * SC * Sm5Ceon001m5Ceon001Teon001m5Ceo * | | SSSSS nXnXnXS |
| | Sm5Ceo | | |
| WV- | mG * SmG * SmC * SmU * SmG * SA * SG * Sm5C * SG * RG * SA * | GGCUGAGCGGAGAAACCCTC | SSSSS SSSRS |
| 30192 | SG * SA * SA * SA * Sm5Ceon001m5Ceon001m5Ceon001Teo * | | SSSSS nXnXnXS |
| | Sm5Ceo | | |
| WV- | mG * SmG * SmC * SmU * SmG * SA * SG * Sm5C * SG * SG * RA * | GGCUGAGCGGAGAAACCCTC | SSSSS SSSSR |
| 30193 | SG * SA * SA * SA * Sm5Ceon001m5Ceon001m5Ceon001Teo * | | SSSSS nXnXnXS |
| | Sm5Ceo | | |
| WV- | mG * SmG * SmC * SmU * SmG * SA * SG * Sm5C * SG * SG * SA * | GGCUGAGCGGAGAAACCCTC | SSSSS SSSSS |
| 30194 | RG * SA * SA * SA * Sm5Ceon001m5Ceon001m5Ceon001Teo * | | RSSSS nXnXnXS |
| | Sm5Ceo | | |
| WV- | mA * SmG * SmG * SmC * SmU * SG * SA * SG * Sm5C * SG * RG * | AGGCUGAGCGGAGAAACCCT | SSSSS SSSSR |
| 30195 | SA * SG * SA * SA * SAeon001m5Ceon001m5Ceon001m5Ceo * STeo | | SSSSS nXnXnXS |
| WV- | mA * SmG * SmG * SmC * SmU * SG * SA * SG * Sm5C * SG * SG * | AGGCUGAGCGGAGAAACCCT | SSSSS SSSSS |
| 30196 | RA * SG * SA * SA * SAeon001m5Ceon001m5Ceon001m5Ceo * STeo | | RSSSS nXnXnXS |
| WV- | mA * SmG * SmG * SmC * SmU * SG * SA * SG * Sm5C * SG * SG * | AGGCUGAGCGGAGAAACCCT | SSSSS SSSSS |
| 30197 | SA * RG * SA * SA * SAeon001m5Ceon001m5Ceon001m5Ceo * STeo | | SRSSS nXnXnXS |
| WV- | mA * SmA * SmG * SmG * SmC * ST * SG * SA * SG * Sm5C * SG * | AAGGCTGAGCGGAGAAACCC | SSSSS SSSSS |
| 30198 | RG * SA * SG * SA * SAeon001Aeon001m5Ceon001m5Ceo * Sm5Ceo | | RSSSS nXnXnXS |
| WV- | mA * SmA * SmG * SmG * SmC * ST * SG * SA * SG * Sm5C * SG * | AAGGCTGAGCGGAGAAACCC | SSSSS SSSSS |
| 30199 | SG * RA * SG * SA * SAeon001Aeon001m5Ceon001m5Ceo * Sm5Ceo | | SRSSS nXnXnXS |
| WV- | mA * SmA * SmG * SmG * SmC * ST * SG * SA * SG * Sm5C * SG * | AAGGCTGAGCGGAGAAACCC | SSSSS SSSSS |
| 30200 | SG * SA * RG * SA * SAeon001Aeon001m5Ceon001m5Ceo * Sm5Ceo | | SSRSS nXnXnXS |
| WV- | mC * SmA * SmA * SmG * SmG * SC * ST * SG * SA * SG * Sm5C * | CAAGGCTGAGCGGAGAAACC | SSSSS SSSSS |
| 30201 | SG * RG * SA * SG * SAeon001Aeon001Aeon001m5Ceo * Sm5Ceo | | SRSSS nXnXnXS |
| WV- | mC * SmA * SmA * SmG * SmG * SC * ST * SG * SA * SG * Sm5C * | CAAGGCTGAGCGGAGAAACC | SSSSS SSSSS |
| 30202 | SG * SG * RA * SG * SAeon001Aeon001Aeon001m5Ceo * Sm5Ceo | | SSRSS nXnXnXS |
| WV- | mC * SmA * SmA * SmG * SmG * SC * ST * SG * SA * SG * Sm5C * | CAAGGCTGAGCGGAGAAACC | SSSSS SSSSS |
| 30203 | SG * SG * SA * RG * SAeon001Aeon001Aeon001m5Ceo * Sm5Ceo | | SSSRS nXnXnXS |
| WV- | mG * SmUn001RmU * SmGn001RmA * ST * SCn001RT * SG * ST * | GUUGATCTGTAGCAGCAGCT | SnRSnRS SnRSSR |
| 30354 | RA * SG * SC * SA * SG * Rm5Ceon001RAeoGeon001Rm5Ceo * STeo | | S SS SR nROnRS |
| WV- | mG * SmUn001RmUmGn001RmA * ST * SCn001RT * SG * ST * RA * | GUUGATCTGTAGCAGCAGCT | SnROnRS SnRSSR |
| 30355 | SG * SC * SA * SG * Rm5Ceon001RAeoGeon001Rm5Ceo * STeo | | S SS SR nROnRS |
| WV- | mG * SmUn001RmUmGn001RmA * ST * SCn001RT * SG * ST * RA | GUUGATCTGTAGCAGCAGCT | SnROnRS SnRSSR |
| 30356 | * SG * SC * SA * SG * Rm5CeoAeoGeon001Rm5Ceo * STeo | | SSSSR OOnRS |
| WV- | mG * SmUn001RmU * SmGmA * ST * SCn001RT * SG * ST * RA * | GUUGATCTGTAGCAGCAGCT | SnRSOS SnRSSR |
| 30357 | SG * SC * SA * SG * Rm5Ceon001RAeoGeon001Rm5Ceo * STeo | | S SS SR nROnRS |
| WV- | Geo * STeoGeom5CeoAeo * SC * SA * SC * SA * SG * ST * SA * | GTGCACACAGTAGATGAGGG | SOOOS SSSSS |
| 31627 | SG * RA * ST * SGeoAeoGeoGeo * SGeo | | SSRSS OOOS |
| WV- | Geo * STeoGeom5CeoAeo * SC * SA * SC * SA * SG * ST * SA * | GTGCACACAGTAGATGAGGG | SOOOS SSSSS |
| 31628 | SG * RA * ST * SmG * SmA * SmG * SmG * SmG | | SSRSS SSSS |
| WV- | mG * SmU * SmG * SmC * SmA * SC * SA * SC * SA * SG * ST * | GUGCACACAGTAGATGAGGG | SSSSS SSSSS |
| 31629 | SA * SG * RA * ST * SGeoAeoGeoGeo * SGeo | | SSRSS OOOS |
| WV- | m5Ceo * SAeom5CeoAeoGeo * ST * RA * SG * SA * ST * SG * SA | CACAGTAGATGAGGGAGCAG | SOOOS RSSSS |
| 31814 | * SG * SG * SG * SAeoGeom5CeoAeo * SGeo | | SSSSS OOOS |
| WV- | m5Ceo * SAeom5CeoAeoGeo * ST * SA * RG * SA * ST * SG * SA | CACAGTAGATGAGGGAGCAG | SOOOS SRSSS |
| 31815 | * SG * SG * SG * SAeoGeom5CeoAeo * SGeo | | SSSSS OOOS |
| WV- | m5Ceo * SAeom5CeoAeoGeo * ST * SA * SG * RA * ST * SG * SA | CACAGTAGATGAGGGAGCAG | SOOOS SSRSS |
| 31816 | * SG * SG * SG * SAeoGeom5CeoAeo * SGeo | | SSSSS OOOS |
| WV- | Aeo * Sm5CeoAeom5CeoAeo * SG * ST * RA * SG * SA * ST * SG | ACACAGTAGATGAGGGAGCA | SOOOS SRSSS |
| 31817 | * SA * SG * SG * SGeoAeoGeom5Ceo * SAeo | | SSSSS OOOS |
| WV- | Aeo * Sm5CeoAeom5CeoAeo * SG * ST * SA * RG * SA * ST * SG | ACACAGTAGATGAGGGAGCA | SOOOS SSRSS |
| 31818 | * SA * SG * SG * SGeoAeoGeom5Ceo * SAeo | | SSSSS OOOS |
| WV- | Aeo * Sm5CeoAeom5CeoAeo * SG * ST * SA * SG * RA * ST * SG | ACACAGTAGATGAGGGAGCA | SOOOS SSSRS |
| 31819 | * SA * SG * SG * SGeoAeoGeom5Ceo * SAeo | | SSSSS OOOS |
| WV- | m5Ceo * SAeom5CeoAeom5Ceo * SA * SG * ST * RA * SG * SA * | CACACAGTAGATGAGGGAGC | SOOOS SSRSS |
| 31820 | ST * SG * SA * SG * SGeoGeoAeoGeo * Sm5Ceo | | SSSSS OOOS |
| WV- | m5Ceo * SAeom5CeoAeom5Ceo * SA * SG * ST * SA * RG * SA * | CACACAGTAGATGAGGGAGC | SOOOS SSSRS |
| 31821 | ST * SG * SA * SG * SGeoGeoAeoGeo * Sm5Ceo | | SSSSS OOOS |
| WV- | m5Ceo * SAeom5CeoAeom5Ceo * SA * SG * ST * SA * SG * RA * | CACACAGTAGATGAGGGAGC | SOOOS SSSSR |
| 31822 | ST * SG * SA * SG * SGeoGeoAeoGeo * Sm5Ceo | | SSSSS OOOS |
| WV- | Geo * Sm5CeoAeom5CeoAeo * SC * SA * SG * ST * RA * SG * SA | GCACACAGTAGATGAGGGAG | SOOOS SSSRS |
| 31823 | * ST * SG * SA * SGeoGeoGeoAeo * SGeo | | SSSSS OOOS |
| WV- | Geo * Sm5CeoAeom5CeoAeo * SC * SA * SG * ST * SA * RG * SA | GCACACAGTAGATGAGGGAG | SOOOS SSSSR |
| 31824 | * ST * SG * SA * SGeoGeoGeoAeo * SGeo | | SSSSS OOOS |
| WV- | Geo * Sm5CeoAeom5CeoAeo * SC * SA * SG * ST * SA * SG * RA | GCACACAGTAGATGAGGGAG | SOOOS SSSSS |
| 31825 | * ST * SG * SA * SGeoGeoGeoAeo * SGeo | | RSSSS OOOS |
| WV- | Teo * SGeom5CeoAeom5Ceo * SA * SC * SA * SG * ST * RA * SG * | TGCACACAGTAGATGAGGGA | SOOOS SSSSR |
| 31826 | SA * ST * SG * SAeoGeoGeoGeo * SAeo | | SSSSS OOOS |
| WV- | Teo * SGeom5CeoAeom5Ceo * SA * SC * SA * SG * ST * SA * RG * | TGCACACAGTAGATGAGGGA | SOOOS SSSSS |
| 31827 | SA * ST * SG * SAeoGeoGeoGeo * SAeo | | RSSSS OOOS |
| WV- | Teo * SGeom5CeoAeom5Ceo * SA * SC * SA * SG * ST * SA * SG * | TGCACACAGTAGATGAGGGA | SOOOS SSSSS |
| 31828 | RA * ST * SG * SAeoGeoGeoGeo * SAeo | | SRSSS OOOS |
| WV- | Geo * STeoGeom5CeoAeo * SC * SA * SC * SA * SG * ST * RA * | GTGCACACAGTAGATGAGGG | SOOOS SSSSS |
| 31829 | SG * SA * ST * SGeoAeoGeoGeo * SGeo | | RSSSS OOOS |
| WV- | Geo * STeoGeom5CeoAeo * SC * SA * SC * SA * SG * ST * SA * | GTGCACACAGTAGATGAGGG | SOOOS SSSSS |
| 31830 | RG * SA * ST * SGeoAeoGeoGeo * SGeo | | SRSSS OOOS |
| WV- | Aeo * SGeoTeoGeom5Ceo * SA * SC * SA * SC * SA * SG * ST * | AGTGCACACAGTAGATGAGG | SOOOS SSSSS |
| 31831 | RA * SG * SA * STeoGeoAeoGeo * SGeo | | SRSSS OOOS |
| WV- | Aeo * SGeoTeoGeom5Ceo * SA * SC * SA * SC * SA * SG * ST * | AGTGCACACAGTAGATGAGG | SOOOS SSSSS |
| 31832 | SA * RG * SA * STeoGeoAeoGeo * SGeo | | SSRSS OOOS |
| WV- | Aeo * SGeoTeoGeom5Ceo * SA * SC * SA * SC * SA * SG * ST * | AGTGCACACAGTAGATGAGG | SOOOS SSSSS |
| 31833 | SA * SG * RA * STeoGeoAeoGeo * SGeo | | SSSRS OOOS |
| WV- | m5Ceo * SAeom5CeoAeoGeo * ST * RA * SG * SA * ST * SG * SA | CACAGTAGATGAGGGAGCAG | SOOOS RSSSS |
| 31834 | * SG * SG * SG * SmA * SmG * SmC * SmA * SmG | | SSSSS SSSS |
| WV- | m5Ceo * SAeom5CeoAeoGeo * ST * SA * RG * SA * ST * SG * SA | CACAGTAGATGAGGGAGCAG | SOOOS SRSSS |
| 31835 | * SG * SG * SG * SmA * SmG * SmC * SmA * SmG | | SSSSS SSSS |
| WV- | m5Ceo * SAeom5CeoAeoGeo * ST * SA * SG * RA * ST * SG * SA | CACAGTAGATGAGGGAGCAG | SOOOS SSRSS |
| 31836 | * SG * SG * SG * SmA * SmG * SmC * SmA * SmG | | SSSSS SSSS |
| WV- | Aeo * Sm5CeoAeom5CeoAeo * SG * ST * RA * SG * SA * ST * SG | ACACAGTAGATGAGGGAGCA | SOOOS SRSSS |
| 31837 | * SA * SG * SG * SmG * SmA * SmG * SmC * SmA | | SSSSS SSSS |
| WV- | Aeo * Sm5CeoAeom5CeoAeo * SG * ST * SA * RG * SA * ST * SG | ACACAGTAGATGAGGGAGCA | SOOOS SSRSS |
| 31838 | * SA * SG * SG * SmG * SmA * SmG * SmC * SmA | | SSSSS SSSS |
| WV- | Aeo * Sm5CeoAeom5CeoAeo * SG * ST * SA * SG * RA * ST * SG | ACACAGTAGATGAGGGAGCA | SOOOS SSSRS |
| 31839 | * SA * SG * SG * SmG * SmA * SmG * SmC * SmA | | SSSSS SSSS |
| WV- | m5Ceo * SAeom5CeoAeom5Ceo * SA * SG * ST * RA * SG * SA * | CACACAGTAGATGAGGGAGC | SOOOS SSRSS |
| 31840 | ST * SG * SA * SG * SmG * SmG * SmA * SmG * SmC | | SSSSS SSSS |
| WV- | m5Ceo * SAeom5CeoAeom5Ceo * SA * SG * ST * SA * RG * SA * | CACACAGTAGATGAGGGAGC | SOOOS SSSRS |
| 31841 | ST * SG * SA * SG * SmG * SmG * SmA * SmG * SmC | | SSSSS SSSS |
| WV- | m5Ceo * SAeom5CeoAeom5Ceo * SA * SG * ST * SA * SG * RA * | CACACAGTAGATGAGGGAGC | SOOOS SSSSR |
| 31842 | ST * SG * SA * SG * SmG * SmG * SmA * SmG * SmC | | SSSSS SSSS |
| WV- | Geo * Sm5CeoAeom5CeoAeo * SC * SA * SG * ST * RA * SG * SA | GCACACAGTAGATGAGGGAG | SOOOS SSSRS |
| 31843 | * ST * SG * SA * SmG * SmG * SmG * SmA * SmG | | SSSSS SSSS |
| WV- | Geo * Sm5CeoAeom5CeoAeo * SC * SA * SG * ST * SA * RG * SA | GCACACAGTAGATGAGGGAG | SOOOS SSSSR |
| 31844 | * ST * SG * SA * SmG * SmG * SmG * SmA * SmG | | SSSSS SSSS |
| WV- | Geo * Sm5CeoAeom5CeoAeo * SC * SA * SG * ST * SA * SG * RA | GCACACAGTAGATGAGGGAG | SOOOS SSSSS |
| 31845 | * ST * SG * SA * SmG * SmG * SmG * SmA * SmG | | RSSSS SSSS |
| WV- | Teo * SGeom5CeoAeom5Ceo * SA * SC * SA * SG * ST * RA * SG * | TGCACACAGTAGATGAGGGA | SOOOS SSSSR |
| 31846 | SA * ST * SG * SmA * SmG * SmG * SmG * SmA | | SSSSS SSSS |
| WV- | Teo * SGeom5CeoAeom5Ceo * SA * SC * SA * SG * ST * SA * RG * | TGCACACAGTAGATGAGGGA | SOOOS SSSSS |
| 31847 | SA * ST * SG * SmA * SmG * SmG * SmG * SmA | | RSSSS SSSS |
| WV- | Teo * SGeom5CeoAeom5Ceo * SA * SC * SA * SG * ST * SA * SG * | TGCACACAGTAGATGAGGGA | SOOOS SSSSS |
| 31848 | RA * ST * SG * SmA * SmG * SmG * SmG * SmA | | SRSSS SSSS |
| WV- | Geo * STeoGeom5CeoAeo * SC * SA * SC * SA * SG * ST * RA * | GTGCACACAGTAGATGAGGG | SOOOS SSSSS |
| 31849 | SG * SA * ST * SmG * SmA * SmG * SmG * SmG | | RSSSS SSSS |
| WV- | Geo * STeoGeom5CeoAeo * SC * SA * SC * SA * SG * ST * SA * | GTGCACACAGTAGATGAGGG | SOOOS SSSSS |
| 31850 | RG * SA * ST * SmG * SmA * SmG * SmG * SmG | | SRSSS SSSS |
| WV- | Aeo * SGeoTeoGeom5Ceo * SA * SC * SA * SC * SA * SG * ST * | AGTGCACACAGTAGAUGAGG | SOOOS SSSSS |
| 31851 | RA * SG * SA * SmU * SmG * SmA * SmG * SmG | | SRSSS SSSS |
| WV- | Aeo * SGeoTeoGeom5Ceo * SA * SC * SA * SC * SA * SG * ST * | AGTGCACACAGTAGAUGAGG | SOOOS SSSSS |
| 31852 | SA * RG * SA * SmU * SmG * SmA * SmG * SmG | | SSRSS SSSS |
| WV- | Aeo * SGeoTeoGeom5Ceo * SA * SC * SA * SC * SA * SG * ST * | AGTGCACACAGTAGAUGAGG | SOOOS SSSSS |
| 31853 | SA * SG * RA * SmU * SmG * SmA * SmG * SmG | | SSSRS SSSS |
| WV- | mG * SmC * SmA * SmC * SmA * SC * SA * SG * ST * RA * SG * | GCACACAGTAGATGAGGGAG | SSSSS SSSRS |
| 31854 | SA * ST * SG * SA * SGeoGeoGeoAeo * SGeo | | SSSSS OOOS |
| WV- | mG * SmC * SmA * SmC * SmA * SC * SA * SG * ST * SA * RG * | GCACACAGTAGATGAGGGAG | SSSSS SSSSR |
| 31855 | SA * ST * SG * SA * SGeoGeoGeoAeo * SGeo | | SSSSS OOOS |
| WV- | mG * SmC * SmA * SmC * SmA * SC * SA * SG * ST * SA * SG * | GCACACAGTAGATGAGGGAG | SSSSS SSSSS |
| 31856 | RA * ST * SG * SA * SGeoGeoGeoAeo * SGeo | | RSSSS OOOS |
| WV- | mU * SmG * SmC * SmA * SmC * SA * SC * SA * SG * ST * RA * | UGCACACAGTAGATGAGGGA | SSSSS SSSSR |
| 31857 | SG * SA * ST * SG * SAeoGeoGeoGeo * SAeo | | SSSSS OOOS |
| WV- | mU * SmG * SmC * SmA * SmC * SA * SC * SA * SG * ST * SA * | UGCACACAGTAGATGAGGGA | SSSSS SSSSS |
| 31858 | RG * SA * ST * SG * SAeoGeoGeoGeo * SAeo | | RSSSS OOOS |
| WV- | mU * SmG * SmC * SmA * SmC * SA * SC * SA * SG * ST * SA * | UGCACACAGTAGATGAGGGA | SSSSS SSSSS |
| 31859 | SG * RA * ST * SG * SAeoGeoGeoGeo * SAeo | | SRSSS OOOS |
| WV- | mG * SmU * SmG * SmC * SmA * SC * SA * SC * SA * SG * ST * | GUGCACACAGTAGATGAGGG | SSSSS SSSSS |
| 31860 | RA * SG * SA * ST * SGeoAeoGeoGeo * SGeo | | RSSSS OOOS |
| WV- | mG * SmU * SmG * SmC * SmA * SC * SA * SC * SA * SG * ST * | GUGCACACAGTAGATGAGGG | SSSSS SSSSS |
| 31861 | SA * RG * SA * ST * SGeoAeoGeoGeo * SGeo | | SRSSS OOOS |
| WV- | mA * SmG * SmU * SmG * SmC * SA * SC * SA * SC * SA * SG * | AGUGCACACAGTAGATGAGG | SSSSS SSSSS |
| 31862 | ST * RA * SG * SA * STeoGeoAeoGeo * SGeo | | SRSSS OOOS |
| WV- | mA * SmG * SmU * SmG * SmC * SA * SC * SA * SC * SA * SG * | AGUGCACACAGTAGATGAGG | SSSSS SSSSS |
| 31863 | ST * SA * RG * SA * STeoGeoAeoGeo * SGeo | | SSRSS OOOS |
| WV- | mA * SmG * SmU * SmG * SmC * SA * SC * SA * SC * SA * SG * | AGUGCACACAGTAGATGAGG | SSSSS SSSSS |
| 31864 | ST * SA * SG * RA * STeoGeoAeoGeo * SGeo | | SSSRS OOOS |
| WV- | Geo * SmUmGmCmA * SC * SA * SC * SA * SG * ST * SA * SG * | GUGCACACAGTAGATGAGGG | SOOOS SSSSS |
| 31865 | RA * ST * SmGmAmGmG * SGeo | | SSRSS OOOS |
| WV- | Geo * SmUmGmCAeo * SC * SA * SC * SA * SG * ST * SA * SG * | GUGCACACAGTAGATGAGGG | SOOOS SSSSS |
| 31866 | RA * ST * SGeomAmGmG * SGeo | | SSRSS OOOS |
| WV- | Geo * SmUmGmCmA * SC * SA * RC * SA * SG * ST * SA * SG * | GUGCACACAGTAGATGAGGG | SOOOS SRSSS |
| 31867 | RA * ST * SmGmAmGmG * SGeo | | SSRSS OOOS |
| WV- | Geo * SmUmGmCAeo * SC * SA * RC * SA * SG * ST * SA * SG * | GUGCACACAGTAGATGAGGG | SOOOS SRSSS |
| 31868 | RA * ST * SGeomAmGmG * SGeo | | SSRSS OOOS |
| WV- | Geo * STeoGeom5CeoAeo * SC * SA * RC * SA * SG * ST * SA * | GTGCACACAGTAGATGAGGG | SOOOS SRSSS |
| 31869 | SG * RA * ST * SGeoAeoGeoGeo * SGeo | | SSRSS OOOS |
| WV- | rArGrCrUrGrCrUrGrCrUrArCrArGrArUrCrArArC | AGCUGCUGCUACAGAUCAAC | OOOOO OOOOO |
| 32539 | | | OOOOO OOOO |
| WV- | rArGrCrUrGrCrUrGrCrUrGrCrArGrArUrCrArArC | AGCUGCUGCUGCAGAUCAAC | OOOOO OOOOO |
| 32540 | | | OOOOO OOOO |
| WV- | mG * SmUn001RmUmGn001RmA * ST * SC * ST * SG * ST * RA * | GUUGATCTGTAGCAGCAGCT | SnROnRS SSSSR |
| 32560 | SG * SC * SA * SG * Rm5Ceon001RAeoGeon001Rm5Ceo * RTeo | | SSSSR nROnRR |
| WV- | mG * SmUn001RmUmGn001RmA * ST * SC * ST * SG * ST * RA * | GUUGATCTGTAGCAGCAGCT | SnROnRS SSSSR |
| 32561 | SG * SC * SA * SG * Rm5Ceon001RAeoGeon001Sm5Ceo * STeo | | SSSSR nROnSS |
| WV- | mG * SmUn001RmUmGn001RmA * ST * SC * ST * SG * ST * RA * | GUUGATCTGTAGCAGCAGCT | SnROnRS SSSSR |
| 32562 | SG * SC * SA * SG * Rm5Ceon001SAeoGeon001Rm5Ceo * STeo | | SSSSR nSOnRS |
| WV- | mG * SmUn001RmUmGn001RmA * ST * SC * ST * SG * ST * RA * | GUUGATCTGTAGCAGCAGCT | SnROnRS SSSSR |
| 32563 | SG * SC * SA * SG * Sm5Ceon001RAeoGeon001Rm5Ceo * STeo | | SSSSS nROnRS |
| WV- | mG * SmUn001RmUmGn001RmA * ST * SC * ST * SG * ST * RA * | GUUGATCTGTAGCAGCAGCT | SnROnRS SSSSR |
| 32564 | SG * SC * SA * RG * Rm5Ceon001RAeoGeon001Rm5Ceo * STeo | | SSSRR nROnRS |
| WV- | mG * SmUn001RmUmGn001RmA * ST * SC * ST * SG * ST * RA * | GUUGATCTGTAGCAGCAGCT | SnROnRS SSSSR |
| 32565 | SG * SC * RA * SG * Rm5Ceon001RAeoGeon001Rm5Ceo * STeo | | SSRSR nROnRS |
| WV- | mG * SmUn001RmUmGn001RmA * ST * SC * ST * SG * ST * RA * | GUUGATCTGTAGCAGCAGCT | SnROnRS SSSSR |
| 32566 | SG * RC * SA * SG * Rm5Ceon001RAeoGeon001Rm5Ceo * STeo | | SRSSR nROnRS |
| WV- | mG * SmUn001RmUmGn001RmA * ST * SC * ST * SG * ST * RA * | GUUGATCTGTAGCAGCAGCT | SnROnRS SSSSR |
| 32567 | RG * SC * SA * SG * Rm5Ceon001RAeoGeon001Rm5Ceo * STeo | | RS SSR nROnRS |
| WV- | mG * SmUn001RmUmGn001RmA * ST * SC * ST * SG * ST * SA * | GUUGATCTGTAGCAGCAGCT | SnROnRS SSSSS |
| 32568 | SG * SC * SA * SG * Rm5Ceon001RAeoGeon001Rm5Ceo * STeo | | S SS SR nROnRS |
| WV- | mG * SmUn001RmUmGn001RmA * ST * SC * ST * SG * RT * RA * | GUUGATCTGTAGCAGCAGCT | SnROnRS SSSRR |
| 32569 | SG * SC * SA * SG * Rm5Ceon001RAeoGeon001Rm5Ceo * STeo | | S SS SR nROnRS |
| WV- | mG * SmUn001RmUmGn001RmA * ST * SC * ST * RG * ST * RA * | GUUGATCTGTAGCAGCAGCT | SnROnRS SSRSR |
| 32570 | SG * SC * SA * SG * Rm5Ceon001RAeoGeon001Rm5Ceo * STeo | | S SS SR nROnRS |
| WV- | mG * SmUn001RmUmGn001RmA * ST * RC * ST * SG * ST * RA * | GUUGATCTGTAGCAGCAGCT | SnROnRS RSSSR |
| 32571 | SG * SC * SA * SG * Rm5Ceon001RAeoGeon001Rm5Ceo * STeo | | S SS SR nROnRS |
| WV- | mG * SmUn001RmUmGn001RmA * RT * SC * ST * SG * ST * RA * | GUUGATCTGTAGCAGCAGCT | SnROnRR SSSSR |
| 32572 | SG * SC * SA * SG * Rm5Ceon001RAeoGeon001Rm5Ceo * STeo | | S SS SR nROnRS |
| WV- | mG * SmUn001RmUmGn001SmA * ST * SC * ST * SG * ST * RA * | GUUGATCTGTAGCAGCAGCT | SnROnSS SSSSR |
| 32573 | SG * SC * SA * SG * Rm5Ceon001RAeoGeon001Rm5Ceo * STeo | | S SS SR nROnRS |
| WV- | mG * SmUn001SmUmGn001RmA * ST * SC * ST * SG * ST * RA * | GUUGATCTGTAGCAGCAGCT | SnSOnRS SSSSR |
| 32574 | SG * SC * SA * SG * Rm5Ceon001RAeoGeon001Rm5Ceo * STeo | | S SS SR nROnRS |
| WV- | mG * RmUn001RmUmGn001RmA * ST * SC * ST * SG * ST * RA * | GUUGATCTGTAGCAGCAGCT | RnROnRS SSSSR |
| 32575 | SG * SC * SA * SG * Rm5Ceon001RAeoGeon001Rm5Ceo * STeo | | S SS SR nROnRS |
| WV- | mGmUn001RmUmGn001RmA * ST * SC * ST * SG * ST * RA * SG | GUUGATCTGTAGCAGCAGCT | OnROnRS SSSSR |
| 32576 | * SC * SA * SG * Rm5Ceon001RAeoGeon001Rm5Ceo * STeo | | S SS SR nROnRS |
| WV- | mUn001RmUmGn001RmA * ST * SC * ST * SG * ST * RA * SG * | UUGATCTGTAGCAGCAGCT | nROnRSS SSSRS |
| 32577 | SC * SA * SG * Rm5Ceon001RAeoGeon001Rm5Ceo * STeo | | SSSRnROnRS |
| WV- | mG * SmG * SmUn001RmUmGn001RmA * ST * SC * ST * SG * ST | GGUUGATCTGTAGCAGCAGCT | SSnROnR SSSSS |
| 32578 | * RA * SG * SC * SA * SG * Rm5Ceon001RAeoGeon001Rm5Ceo * | | RS SSS RnROnRS |
| | STeo | | |
| WV- | mG * SmUn001mUmGn001mA * ST * SC * ST * SG * ST * RA * SG | GUUGATCTGTAGCAGCAGCT | SnXOnXS SSSSR |
| 32685 | * SC * SA * SG * Rm5Ceon001AeoGeon001m5Ceo * STeo | | S SS SR nXOnXS |
| WV- | mG * SmUn001mU * SmGmA * ST * SC * ST * SG * ST * RA * SG | GUUGATCTGTAGCAGCAGCT | SnXSOS SSSSR |
| 32686 | * SC * SA * SG * Rm5Ceon001AeoGeon001m5Ceo * STeo | | S SS SR nXOnXS |
| WV- | m5Ceo * SAeon001m5Ceon001Aeon001Geo * ST * RA * SG * SA * | CACAGTAGATGAGGGAGCAG | SnXnXnXS RSSSS |
| 33091 | ST * SG * SA * SG * SG * SG * SAeoGeom5CeoAeo * SGeo | | SSSSS OOOS |
| WV- | m5Ceo * SAeon001m5Ceon001Aeon001Geo * ST * SA * RG * SA * | CACAGTAGATGAGGGAGCAG | SnXnXnXS SRSSS |
| 33092 | ST * SG * SA * SG * SG * SG * SAeoGeom5CeoAeo * SGeo | | SSSSS OOOS |
| WV- | m5Ceo * SAeon001m5Ceon001Aeon001Geo * ST * SA * SG * RA * | CACAGTAGATGAGGGAGCAG | SnXnXnXS SSRSS |
| 33093 | ST * SG * SA * SG * SG * SG * SAeoGeom5CeoAeo * SGeo | | SSSSS OOOS |
| WV- | Aeo * Sm5Ceon001Aeon001m5Ceon001Aeo * SG * ST * RA * SG * | ACACAGTAGATGAGGGAGCA | SnXnXnXS SRSSS |
| 33094 | SA * ST * SG * SA * SG * SG * SGeoAeoGeom5Ceo * SAeo | | SSSSS OOOS |
| WV- | Aeo * Sm5Ceon001Aeon001m5Ceon001Aeo * SG * ST * SA * RG * | ACACAGTAGATGAGGGAGCA | SnXnXnXS SSRSS |
| 33095 | SA * ST * SG * SA * SG * SG * SGeoAeoGeom5Ceo * SAeo | | SSSSS OOOS |
| WV- | Aeo * Sm5Ceon001Aeon001m5Ceon001Aeo * SG * ST * SA * SG * | ACACAGTAGATGAGGGAGCA | SnXnXnXS SSSRS |
| 33096 | RA * ST * SG * SA * SG * SG * SGeoAeoGeom5Ceo * SAeo | | SSSSS OOOS |
| WV- | m5Ceo * SAeon001m5Ceon001Aeon001m5Ceo * SA * SG * ST * RA | CACACAGTAGATGAGGGAGC | SnXnXnXS SSRSS |
| 33097 | * SG * SA * ST * SG * SA * SG * SGeoGeoAeoGeo * Sm5Ceo | | SSSSS OOOS |
| WV- | m5Ceo * SAeon001m5Ceon001Aeon001m5Ceo * SA * SG * ST * SA | CACACAGTAGATGAGGGAGC | SnXnXnXS SSSRS |
| 33098 | * RG * SA * ST * SG * SA * SG * SGeoGeoAeoGeo * Sm5Ceo | | SSSSS OOOS |
| WV- | m5Ceo * SAeon001m5Ceon001Aeon001m5Ceo * SA * SG * ST * SA | CACACAGTAGATGAGGGAGC | SnXnXnXS SSSSR |
| 33099 | * SG * RA * ST * SG * SA * SG * SGeoGeoAeoGeo * Sm5Ceo | | SSSSS OOOS |
| WV- | Geo * Sm5Ceon001Aeon001m5Ceon001Aeo * SC * SA * SG * ST * | GCACACAGTAGATGAGGGAG | SnXnXnXS SSSRS |
| 33100 | RA * SG * SA * ST * SG * SA * SGeoGeoGeoAeo * SGeo | | SSSSS OOOS |
| WV- | Geo * Sm5Ceon001Aeon001m5Ceon001Aeo * SC * SA * SG * ST * | GCACACAGTAGATGAGGGAG | SnXnXnXS SSSSR |
| 33101 | SA * RG * SA * ST * SG * SA * SGeoGeoGeoAeo * SGeo | | SSSSS OOOS |
| WV- | Geo * Sm5Ceon001Aeon001m5Ceon001Aeo * SC * SA * SG * ST * | GCACACAGTAGATGAGGGAG | SnXnXnXS SSSSS |
| 33102 | SA * SG * RA * ST * SG * SA * SGeoGeoGeoAeo * SGeo | | RSSSS OOOS |
| WV- | Teo * SGeon001m5Ceon001Aeon001m5Ceo * SA * SC * SA * SG * | TGCACACAGTAGATGAGGGA | SnXnXnXS SSSSR |
| 33103 | ST * RA * SG * SA * ST * SG * SAeoGeoGeoGeo * SAeo | | SSSSS OOOS |
| WV- | Teo * SGeon001m5Ceon001Aeon001m5Ceo * SA * SC * SA * SG * | TGCACACAGTAGATGAGGGA | SnXnXnXS SSSSS |
| 33104 | ST * SA * RG * SA * ST * SG * SAeoGeoGeoGeo * SAeo | | RSSSS OOOS |
| WV- | Teo * SGeon001m5Ceon001Aeon001m5Ceo * SA * SC * SA * SG * | TGCACACAGTAGATGAGGGA | SnXnXnXS SSSSS |
| 33105 | ST * SA * SG * RA * ST * SG * SAeoGeoGeoGeo * SAeo | | SRSSS OOOS |
| WV- | Geo * STeon001Geon001m5Ceon001Aeo * SC * SA * SC * SA * SG | GTGCACACAGTAGATGAGGG | SnXnXnXS SSSSS |
| 33106 | * ST * RA * SG * SA * ST * SGeoAeoGeoGeo * SGeo | | RSSSS OOOS |
| WV- | Geo * STeon001Geon001m5Ceon001Aeo * SC * SA * SC * SA * SG | GTGCACACAGTAGATGAGGG | SnXnXnXS SSSSS |
| 33107 | * ST * SA * RG * SA * ST * SGeoAeoGeoGeo * SGeo | | SRSSS OOOS |
| WV- | Geo * STeon001Geon001m5Ceon001Aeo * SC * SA * SC * SA * SG | GTGCACACAGTAGATGAGGG | SnXnXnXS SSSSS |
| 33108 | * ST * SA * SG * RA * ST * SGeoAeoGeoGeo * SGeo | | SSRSS OOOS |
| WV- | Aeo * SGeon001Teon001Geon001m5Ceo * SA * SC * SA * SC * SA | AGTGCACACAGTAGATGAGG | SnXnXnXS SSSSS |
| 33109 | * SG * ST * RA * SG * SA * STeoGeoAeoGeo * SGeo | | SRSSS OOOS |
| WV- | Aeo * SGeon001Teon001Geon001m5Ceo * SA * SC * SA * SC * SA | AGTGCACACAGTAGATGAGG | SnXnXnXS SSSSS |
| 33110 | * SG * ST * SA * RG * SA * STeoGeoAeoGeo * SGeo | | SSRSS OOOS |
| WV- | Aeo * SGeon001Teon001Geon001m5Ceo * SA * SC * SA * SC * SA | AGTGCACACAGTAGATGAGG | SnXnXnXS SSSSS |
| 33111 | * SG * ST * SA * SG * RA * STeoGeoAeoGeo * SGeo | | SSSRS OOOS |
| WV- | m5Ceo * SAeom5CeoAeoGeo * ST * RA * SG * SA * ST * SG * SA | CACAGTAGATGAGGGAGCAG | SOOOS RSSSS |
| 33112 | * SG * SG * SG * SAeon001Geon001m5Ceon001Aeo * SGeo | | SSSSS nXnXnXS |
| WV- | m5Ceo * SAeom5CeoAeoGeo * ST * SA * RG * SA * ST * SG * SA | CACAGTAGATGAGGGAGCAG | SOOOS SRSSS |
| 33113 | * SG * SG * SG * SAeon001Geon001m5Ceon001Aeo * SGeo | | SSSSS nXnXnXS |
| WV- | m5Ceo * SAeom5CeoAeoGeo * ST * SA * SG * RA * ST * SG * SA | CACAGTAGATGAGGGAGCAG | SOOOS SSRSS |
| 33114 | * SG * SG * SG * SAeon001Geon001m5Ceon001Aeo * SGeo | | SSSSS nXnXnXS |
| WV- | Aeo * Sm5CeoAeom5CeoAeo * SG * ST * RA * SG * SA * ST * SG | ACACAGTAGATGAGGGAGCA | SOOOS SRSSS |
| 33115 | * SA * SG * SG * SGeon001Aeon001Geon001m5Ceo * SAeo | | SSSSS nXnXnXS |
| WV- | Aeo * Sm5CeoAeom5CeoAeo * SG * ST * SA * RG * SA * ST * SG | ACACAGTAGATGAGGGAGCA | SOOOS SSRSS |
| 33116 | * SA * SG * SG * SGeon001Aeon001Geon001m5Ceo * SAeo | | SSSSS nXnXnXS |
| WV- | Aeo * Sm5CeoAeom5CeoAeo * SG * ST * SA * SG * RA * ST * SG | ACACAGTAGATGAGGGAGCA | SOOOS SSSRS |
| 33117 | * SA * SG * SG * SGeon001Aeon001Geon001m5Ceo * SAeo | | SSSSS nXnXnXS |
| WV- | m5Ceo * SAeom5CeoAeom5Ceo * SA * SG * ST * RA * SG * SA * | CACACAGTAGATGAGGGAGC | SOOOS SSRSS |
| 33118 | ST * SG * SA * SG * SGeon001Geon001Aeon001Geo * Sm5Ceo | | SSSSS nXnXnXS |
| WV- | m5Ceo * SAeom5CeoAeom5Ceo * SA * SG * ST * SA * RG * SA * | CACACAGTAGATGAGGGAGC | SOOOS SSSRS |
| 33119 | ST * SG * SA * SG * SGeon001Geon001Aeon001Geo * Sm5Ceo | | SSSSS nXnXnXS |
| WV- | m5Ceo * SAeom5CeoAeom5Ceo * SA * SG * ST * SA * SG * RA * | CACACAGTAGATGAGGGAGC | SOOOS SSSSR |
| 33120 | ST * SG * SA * SG * SGeon001Geon001Aeon001Geo * Sm5Ceo | | SSSSS nXnXnXS |
| WV- | Geo * Sm5CeoAeom5CeoAeo * SC * SA * SG * ST * RA * SG * SA | GCACACAGTAGATGAGGGAG | SOOOS SSSRS |
| 33121 | * ST * SG * SA * SGeon001Geon001Geon001Aeo * SGeo | | SSSSS nXnXnXS |
| WV- | Geo * Sm5CeoAeom5CeoAeo * SC * SA * SG * ST * SA * RG * SA | GCACACAGTAGATGAGGGAG | SOOOS SSSSR |
| 33122 | * ST * SG * SA * SGeon001Geon001Geon001Aeo * SGeo | | SSSSS nXnXnXS |
| WV- | Geo * Sm5CeoAeom5CeoAeo * SC * SA * SG * ST * SA * SG * RA | GCACACAGTAGATGAGGGAG | SOOOS SSSSS |
| 33123 | * ST * SG * SA * SGeon001Geon001Geon001Aeo * SGeo | | RSSSS nXnXnXS |
| WV- | Teo * SGeom5CeoAeom5Ceo * SA * SC * SA * SG * ST * RA * SG * | TGCACACAGTAGATGAGGGA | SOOOS SSSSR |
| 33124 | SA * ST * SG * SAeon001Geon001Geon001Geo * SAeo | | SSSSS nXnXnXS |
| WV- | Teo * SGeom5CeoAeom5Ceo * SA * SC * SA * SG * ST * SA * RG * | TGCACACAGTAGATGAGGGA | SOOOS SSSSS |
| 33125 | SA * ST * SG * SAeon001Geon001Geon001Geo * SAeo | | RSSSS nXnXnXS |
| WV- | Teo * SGeom5CeoAeom5Ceo * SA * SC * SA * SG * ST * SA * SG * | TGCACACAGTAGATGAGGGA | SOOOS SSSSS |
| 33126 | RA * ST * SG * SAeon001Geon001Geon001Geo * SAeo | | SRSSS nXnXnXS |
| WV- | Geo * STeoGeom5CeoAeo * SC * SA * SC * SA * SG * ST * RA * | GTGCACACAGTAGATGAGGG | SOOOS SSSSS |
| 33127 | SG * SA * ST * SGeon001Aeon001Geon001Geo * SGeo | | RSSSS nXnXnXS |
| WV- | Geo * STeoGeom5CeoAeo * SC * SA * SC * SA * SG * ST * SA * | GTGCACACAGTAGATGAGGG | SOOOS SSSSS |
| 33128 | RG * SA * ST * SGeon001Aeon001Geon001Geo * SGeo | | SRSSS nXnXnXS |
| WV- | Geo * STeoGeom5CeoAeo * SC * SA * SC * SA * SG * ST * SA * | GTGCACACAGTAGATGAGGG | SOOOS SSSSS |
| 33129 | SG * RA * ST * SGeon001Aeon001Geon001Geo * SGeo | | SSRSS nXnXnXS |
| WV- | Aeo * SGeoTeoGeom5Ceo * SA * SC * SA * SC * SA * SG * ST * | AGTGCACACAGTAGATGAGG | SOOOS SSSSS |
| 33130 | RA * SG * SA * STeon001Geon001Aeon001Geo * SGeo | | SRSSS nXnXnXS |
| WV- | Aeo * SGeoTeoGeom5Ceo * SA * SC * SA * SC * SA * SG * ST * | AGTGCACACAGTAGATGAGG | SOOOS SSSSS |
| 33131 | SA * RG * SA * STeon001Geon001Aeon001Geo * SGeo | | SSRSS nXnXnXS |
| WV- | Aeo * SGeoTeoGeom5Ceo * SA * SC * SA * SC * SA * SG * ST * | AGTGCACACAGTAGATGAGG | SOOOS SSSSS |
| 33132 | SA * SG * RA * STeon001Geon001Aeon001Geo * SGeo | | SSSRS nXnXnXS |
| WV- | m5Ceo * SAeon001m5Ceon001Aeon001Geo * ST * RA * SG * SA * | CACAGTAGATGAGGGAGCAG | SnXnXnXS RSSSS |
| 33133 | ST * SG * SA * SG * SG * SG * SmA * SmG * SmC * SmA * SmG | | SSSSS SSSS |
| WV- | m5Ceo * SAeon001m5Ceon001Aeon001Geo * ST * SA * RG * SA * | CACAGTAGATGAGGGAGCAG | SnXnXnXS SRSSS |
| 33134 | ST * SG * SA * SG * SG * SG * SmA * SmG * SmC * SmA * SmG | | SSSSS SSSS |
| WV- | m5Ceo * SAeon001m5Ceon001Aeon001Geo * ST * SA * SG * RA * | CACAGTAGATGAGGGAGCAG | SnXnXnXS SSRSS |
| 33135 | ST * SG * SA * SG * SG * SG * SmA * SmG * SmC * SmA * SmG | | SSSSS SSSS |
| WV- | Aeo * Sm5Ceon001Aeon001m5Ceon001Aeo * SG * ST * RA * SG * | ACACAGTAGATGAGGGAGCA | SnXnXnXS SRSSS |
| 33136 | SA * ST * SG * SA * SG * SG * SmG * SmA * SmG * SmC * SmA | | SSSSS SSSS |
| WV- | Aeo * Sm5Ceon001Aeon001m5Ceon001Aeo * SG * ST * SA * RG * | ACACAGTAGATGAGGGAGCA | SnXnXnXS SSRSS |
| 33137 | SA * ST * SG * SA * SG * SG * SmG * SmA * SmG * SmC * SmA | | SSSSS SSSS |
| WV- | Aeo * Sm5Ceon001Aeon001m5Ceon001Aeo * SG * ST * SA * SG * | ACACAGTAGATGAGGGAGCA | SnXnXnXS SSSRS |
| 33138 | RA * ST * SG * SA * SG * SG * SmG * SmA * SmG * SmC * SmA | | SSSSS SSSS |
| WV- | m5Ceo * SAeon001m5Ceon001Aeon001m5Ceo * SA * SG * ST * RA | CACACAGTAGATGAGGGAGC | SnXnXnXS SSRSS |
| 33139 | * SG * SA * ST * SG * SA * SG * SmG * SmG * SmA * SmG * SmC | | SSSSS SSSS |
| WV- | m5Ceo * SAeon001m5Ceon001Aeon001m5Ceo * SA * SG * ST * SA | CACACAGTAGATGAGGGAGC | SnXnXnXS SSSRS |
| 33140 | * RG * SA * ST * SG * SA * SG * SmG * SmG * SmA * SmG * SmC | | SSSSS SSSS |
| WV- | m5Ceo * SAeon001m5Ceon001Aeon001m5Ceo * SA * SG * ST * SA | CACACAGTAGATGAGGGAGC | SnXnXnXS SSSSR |
| 33141 | * SG * RA * ST * SG * SA * SG * SmG * SmG * SmA * SmG * SmC | | SSSSS SSSS |
| WV- | Geo * Sm5Ceon001Aeon001m5Ceon001Aeo * SC * SA * SG * ST * | GCACACAGTAGATGAGGGAG | SnXnXnXS SSSRS |
| 33142 | RA * SG * SA * ST * SG * SA * SmG * SmG * SmG * SmA * SmG | | SSSSS SSSS |
| WV- | Geo * Sm5Ceon001Aeon001m5Ceon001Aeo * SC * SA * SG * ST * | GCACACAGTAGATGAGGGAG | SnXnXnXS SSSSR |
| 33143 | SA * RG * SA * ST * SG * SA * SmG * SmG * SmG * SmA * SmG | | SSSSS SSSS |
| WV- | Geo * Sm5Ceon001Aeon001m5Ceon001Aeo * SC * SA * SG * ST * | GCACACAGTAGATGAGGGAG | SnXnXnXS SSSSS |
| 33144 | SA * SG * RA * ST * SG * SA * SmG * SmG * SmG * SmA * SmG | | RSSSS SSSS |
| WV- | Teo * SGeon001m5Ceon001Aeon001m5Ceo * SA * SC * SA * SG * | TGCACACAGTAGATGAGGGA | SnXnXnXS SSSSR |
| 33145 | ST * RA * SG * SA * ST * SG * SmA * SmG * SmG * SmG * SmA | | SSSSS SSSS |
| WV- | Teo * SGeon001m5Ceon001Aeon001m5Ceo * SA * SC * SA * SG * | TGCACACAGTAGATGAGGGA | SnXnXnXS SSSSS |
| 33146 | ST * SA * RG * SA * ST * SG * SmA * SmG * SmG * SmG * SmA | | RSSSS SSSS |
| WV- | Teo * SGeon001m5Ceon001Aeon001m5Ceo * SA * SC * SA * SG * | TGCACACAGTAGATGAGGGA | SnXnXnXS SSSSS |
| 33147 | ST * SA * SG * RA * ST * SG * SmA * SmG * SmG * SmG * SmA | | SRSSS SSSS |
| WV- | Geo * STeon001Geon001m5Ceon001Aeo * SC * SA * SC * SA * SG | GTGCACACAGTAGATGAGGG | SnXnXnXS SSSSS |
| 33148 | * ST * RA * SG * SA * ST * SmG * SmA * SmG * SmG * SmG | | RSSSS SSSS |
| WV- | Geo * STeon001Geon001m5Ceon001Aeo * SC * SA * SC * SA * SG | GTGCACACAGTAGATGAGGG | SnXnXnXS SSSSS |
| 33149 | * ST * SA * RG * SA * ST * SmG * SmA * SmG * SmG * SmG | | SRSSS SSSS |
| WV- | Geo * STeon001Geon001m5Ceon001Aeo * SC * SA * SC * SA * SG | GTGCACACAGTAGATGAGGG | SnXnXnXS SSSSS |
| 33150 | * ST * SA * SG * RA * ST * SmG * SmA * SmG * SmG * SmG | | SSRSS SSSS |
| WV- | Aeo * SGeon001Teon001Geon001m5Ceo * SA * SC * SA * SC * SA | AGTGCACACAGTAGAUGAGG | SnXnXnXS SSSSS |
| 33151 | * SG * ST * RA * SG * SA * SmU * SmG * SmA * SmG * SmG | | SRSSS SSSS |
| WV- | Aeo * SGeon001Teon001Geon001m5Ceo * SA * SC * SA * SC * SA | AGTGCACACAGTAGAUGAGG | SnXnXnXS SSSSS |
| 33152 | * SG * ST * SA * RG * SA * SmU * SmG * SmA * SmG * SmG | | SSRSS SSSS |
| WV- | Aeo * SGeon001Teon001Geon001m5Ceo * SA * SC * SA * SC * SA | AGTGCACACAGTAGAUGAGG | SnXnXnXS SSSSS |
| 33153 | * SG * ST * SA * SG * RA * SmU * SmG * SmA * SmG * SmG | | SSSRS SSSS |
| WV- | mG * SmC * SmA * SmC * SmA * SC * SA * SG * ST * RA * SG * | GCACACAGTAGATGAGGGAG | SSSSS SSSRS |
| 33154 | SA * ST * SG * SA * SGeon001Geon001Geon001Aeo * SGeo | | SSSSS nXnXnXS |
| WV- | mG * SmC * SmA * SmC * SmA * SC * SA * SG * ST * SA * RG * | GCACACAGTAGATGAGGGAG | SSSSS SSSSR |
| 33155 | SA * ST * SG * SA * SGeon001Geon001Geon001Aeo * SGeo | | SSSSS nXnXnXS |
| WV- | mG * SmC * SmA * SmC * SmA * SC * SA * SG * ST * SA * SG * | GCACACAGTAGATGAGGGAG | SSSSS SSSSS |
| 33156 | RA * ST * SG * SA * SGeon001Geon001Geon001Aeo * SGeo | | RSSSS nXnXnXS |
| WV- | mU * SmG * SmC * SmA * SmC * SA * SC * SA * SG * ST * RA * | UGCACACAGTAGATGAGGGA | SSSSS SSSSR |
| 33157 | SG * SA * ST * SG * SAeon001Geon001Geon001Geo * SAeo | | SSSSS nXnXnXS |
| WV- | mU * SmG * SmC * SmA * SmC * SA * SC * SA * SG * ST * SA * | UGCACACAGTAGATGAGGGA | SSSSS SSSSS |
| 33158 | RG * SA * ST * SG * SAeon001Geon001Geon001Geo * SAeo | | RSSSS nXnXnXS |
| WV- | mU * SmG * SmC * SmA * SmC * SA * SC * SA * SG * ST * SA * | UGCACACAGTAGATGAGGGA | SSSSS SSSSS |
| 33159 | SG * RA * ST * SG * SAeon001Geon001Geon001Geo * SAeo | | SRSSS nXnXnXS |
| WV- | mG * SmU * SmG * SmC * SmA * SC * SA * SC * SA * SG * ST * | GUGCACACAGTAGATGAGGG | SSSSS SSSSS |
| 33160 | RA * SG * SA * ST * SGeon001Aeon001Geon001Geo * SGeo | | RSSSS nXnXnXS |
| WV- | mG * SmU * SmG * SmC * SmA * SC * SA * SC * SA * SG * ST * | GUGCACACAGTAGATGAGGG | SSSSS SSSSS |
| 33161 | SA * RG * SA * ST * SGeon001Aeon001Geon001Geo * SGeo | | SRSSS nXnXnXS |
| WV- | mG * SmU * SmG * SmC * SmA * SC * SA * SC * SA * SG * ST * | GUGCACACAGTAGATGAGGG | SSSSS SSSSS |
| 33162 | SA * SG * RA * ST * SGeon001Aeon001Geon001Geo * SGeo | | SSRSS nXnXnXS |
| WV- | mA * SmG * SmU * SmG * SmC * SA * SC * SA * SC * SA * SG * | AGUGCACACAGTAGATGAGG | SSSSS SSSSS |
| 33163 | ST * RA * SG * SA * STeon001Geon001Aeon001Geo * SGeo | | SRSSS nXnXnXS |
| WV- | mA * SmG * SmU * SmG * SmC * SA * SC * SA * SC * SA * SG * | AGUGCACACAGTAGATGAGG | SSSSS SSSSS |
| 33164 | ST * SA * RG * SA * STeon001Geon001Aeon001Geo * SGeo | | SSRSS nXnXnXS |
| WV- | mA * SmG * SmU * SmG * SmC * SA * SC * SA * SC * SA * SG * | AGUGCACACAGTAGATGAGG | SSSSS SSSSS |
| 33165 | ST * SA * SG * RA * STeon001Geon001Aeon001Geo * SGeo | | SSSRS nXnXnXS |
| WV- | Geo * SmUn001mGn001mCn001mA * SC * SA * SC * SA * SG * ST | GUGCACACAGTAGATGAGGG | SnXnXnXS SSSSS |
| 33166 | * SA * SG * RA * ST * SmGmAmGmG * SGeo | | SSRSS OOOS |
| WV- | Geo * SmUmGmCmA * SC * SA * SC * SA * SG * ST * SA * SG * | GUGCACACAGTAGATGAGGG | SOOOS SSSSS |
| 33167 | RA * ST * SmGn001mAn001mGn001mG * SGeo | | SSRSS nXnXnXS |
| WV- | Geo * SmUn001mGn001mCn001Aeo * SC * SA * SC * SA * SG * ST | GUGCACACAGTAGATGAGGG | SnXnXnXS SSSSS |
| 33168 | * SA * SG * RA * ST * SGeomAmGmG * SGeo | | SSRSS OOOS |
| WV- | Geo * SmUmGmCAeo * SC * SA * SC * SA * SG * ST * SA * SG * | GUGCACACAGTAGATGAGGG | SOOOS SSSSS |
| 33169 | RA * ST * SGeon001mAn001mGn001mG * SGeo | | SSRSS nXnXnXS |
| WV- | Geo * SmUn001mGn001mCn001mA * SC * SA * RC * SA * SG * ST | GUGCACACAGTAGATGAGGG | SnXnXnXS SRSSS |
| 33170 | * SA * SG * RA * ST * SmGmAmGmG * SGeo | | SSRSS OOOS |
| WV- | Geo * SmUmGmCmA * SC * SA * RC * SA * SG * ST * SA * SG * | GUGCACACAGTAGATGAGGG | SOOOS SRSSS |
| 33171 | RA * ST * SmGn001mAn001mGn001mG * SGeo | | SSRSS nXnXnXS |
| WV- | Geo * SmUn001mGn001mCn001Aeo * SC * SA * RC * SA * SG * ST | GUGCACACAGTAGATGAGGG | SnXnXnXS SRSSS |
| 33172 | * SA * SG * RA * ST * SGeomAmGmG * SGeo | | SSRSS OOOS |
| WV- | Geo * SmUmGmCAeo * SC * SA * RC * SA * SG * ST * SA * SG * | GUGCACACAGTAGATGAGGG | SOOOS SRSSS |
| 33173 | RA * ST * SGeon001mAn001mGn001mG * SGeo | | SSRSS nXnXnXS |
| WV- | Geo * STeon001Geon001m5Ceon001Aeo * SC * SA * RC * SA * SG | GTGCACACAGTAGATGAGGG | SnXnXnXS SRSSS |
| 33174 | * ST * SA * SG * RA * ST * SGeoAeoGeoGeo * SGeo | | SSRSS OOOS |
| WV- | Geo * STeoGeom5CeoAeo * SC * SA * RC * SA * SG * ST * SA * | GTGCACACAGTAGATGAGGG | SOOOS SRSSS |
| 33175 | SG * RA * ST * SGeon001Aeon001Geon001Geo * SGeo | | SSRSS nXnXnXS |
| WV- | Mod039L001 m5CeoAeoTeoGeoTeoCACCCAGCAGAeo m5Ceo | CATGTCACCCAGCAGACCAG | OSOOO SSRSS |
| 33213 | m5CeoAeoGeo | | RSSRS SOOOS |
| WV- | mA m5CeoTeo m5Ceo mACCCACT m5CG m5CC mA mc m5Ceo | ACTCACCCACTCGCCACCGC | SOOOS RSRSS |
| 34213 | mG mC | | SSRSS SSSS |
| WV- | mG * SmGmCmAmC * SA * SA * SG * SG * SG * SC * RA * SC * | GGCACAAGGG CACAGACUUC | SOOOSSSSSS |
| 2732 | RA * SG * SmAmCmUmU * SmC | | RSRSSOOOS |

Notes:
Description, Base Sequence and Stereochemistry/Linkage, due to their length, may be divided into multiple lines in Table 1. Unless otherwise specified, all oligonucleotides in Table 1 are single-stranded. As appreciated by those skilled in the art, nucleoside units are unmodified and contain unmodified nucleobases and 2'-deoxy sugars unless otherwise indicated with modifications (e.g., modified with r, m, m5, eo, etc.); linkages, unless otherwise indicated, are natural phosphate linkages; and acidic/basic groups may independently exist in their salt forms. As appreciated by those skilled in the art, when no intemucleotidic linkage is specified between two nucleoside units, the intemucleotidic linkage is a phosphodiester linkage (natural phosphate linkage), and unless indicated otherwise a sugar is a natural DNA sugar which comprises no substitution at the 2' position (two—H at 2'-carbon). Moieties and modifications in oligonucleotides (or other compounds, e.g., those useful for preparing provided oligonucleotides comprising these moieties or modifications):

### m: 2′-OMe;

m5: methyl at 5-position of C (nucleobase is 5-methylcytosine);
m5Ceo: 5-methyl 2′-O-methoxyethyl C;
m5mC: 5-methyl 2′-OMe C;
m5lC: methyl at 5-position of C (nucleobase is 5-methylcytosine) and sugar is a LNA sugar;
eo: 2′-MOE (2′-OCH₂CH₂OCH₃);

### f: 2′-F;

### r: 2′-OH;

O, PO: phosphodiester (phosphate). It can be an end group, or a linkage, e.g., a linkage between a linker and an oligonucleotide chain, an internucleotidic linkage (a natural phosphate linkage), etc. Phosphodiesters are typically indicated with “O” in the Stereochemistry/Linkage column and are typically not marked in the Description column (if it is an end group, e.g., a 5′-end group, it is indicated in the Description and typically not in Stereochemistry/Linkage); if no linkage is indicated in the description column, it is typically a phosphodiester unless otherwise indicated. Note that a phosphate linkage between a linker (e.g., L001) and an oligonucleotide chain may not be marked in the Description column, and may not be indicated with “O” in the Stereochemistry/Linkage column. For example, in the Description of WV-10631 (Mod012L001mG * SmUmGmCmA . . . ), the phosphodiester linkage between L001 and the oligonucleotide chain (starting with mG * SmUmGmCmA . . . ) is not marked; this internucleotidic linkage is indicated in Stereochemistry/Linkage with the first “O” in: OSOOO . . . *, PS: Phosphorothioate. It can be an end group (if it is an end group, e.g., a 5′-end group, it is indicated in the Description and typically not in Stereochemistry/Linkage), or a linkage, e.g., a linkage between linker (e.g., L001) and an oligonucleotide chain, an internucleotidic linkage (a phosphorothioate internucleotidic linkage), etc.
R, Rp: Phosphorothioate in the Rp conformation. Note that * R in Description indicates a single phosphorothioate linkage in the Rp conformation;
S, Sp: Phosphorothioate in the Sp conformation. Note that * S in Description indicates a single phosphorothioate linkage in the Sp conformation;
X: stereorandom phosphorothioate;
l: LNA sugar;
n001:

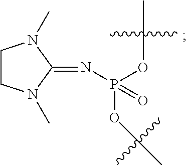 [SMILES: *OP(=O)(N=C1N(C)CCN1C)O*]

nX or Xn: stereorandom n001;
n001R or nR: n001 in the Rp configuration;
n001S or nS: n001 in the Sp configuration;
L001: —NH—(CH₂)₆— linker (also known as a C6 linker, C6 amine linker or C6 amino linker), connected to Mod, if any, through —NH—, and the 5′-end or 3′-end of the oligonucleotide chain through either a phosphate linkage (—O—P(O)(OH)—O—, which may exist as a salt form, and may be indicated as O or PO) or a phosphorothioate linkage (—O—P(O)(SH)—O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration) as indicated at the —CH₂— connecting site. If no Mod is present, L001 is connected to —H through —NH—;
L004: linker having the structure of —NH(CH₂)₄CH(CH₂OH)CH₂—, wherein —NH— is connected to Mod (through —C(O)—) or —H, and the —CH₂— connecting site is connected to an oligonucleotide chain (e.g., at the 3′-end) through a linkage, e.g., phosphodiester (—O—P(O)(OH)—O—, which may exist as a salt form, and may be indicated as O or PO), phosphorothioate (—O—P(O)(SH)—O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration), or phosphorodithioate (—O—P(S)(SH)—O—, which may exist as a salt form, and may be indicated as PS2 or: or D) linkage. For example, an asterisk immediately preceding a L004 (e.g., *L004) indicates that the linkage is a phosphorothioate linkage, and the absence of an asterisk immediately preceding L004 indicates that the linkage is a phosphodiester linkage. For example, in an oligonucleotide which terminates in . . . mAL004, the linker L004 is connected (via the —CH₂— site) through a phosphodiester linkage to the 3′ position of the 3′-terminal sugar (which is 2′-OMe modified and connected to the nucleobase A), and the L004 linker is connected via —NH— to —H. Similarly, in one or more oligonucleotides, the L004 linker is connected (via the —CH₂— site) through the phosphodiester linkage to the 3′ position of the 3′-terminal sugar, and the L004 is connected via —NH— to, e.g., Mod012, Mod085, Mod086, etc.; Mod012 (with —C(O)— connecting to —NH— of a linker such as L001 or L004):

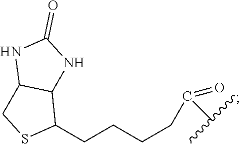 [SMILES: *C(=O)CCCCC1SCC2NC(=O)NC21]

Mod039 (with —C(O)— connecting to —NH— of a linker such as L001 or L004):

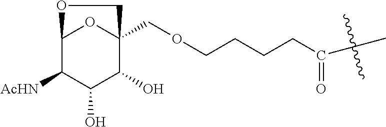 [SMILES: *C(=O)CCCCOC[C@@]12CO[C@@H](O1)[C@H](NC(C)=O)[C@@H](O)[C@H]2O]

Mod062 (with —NH— connecting to —C(O)— of a linker such as L008):

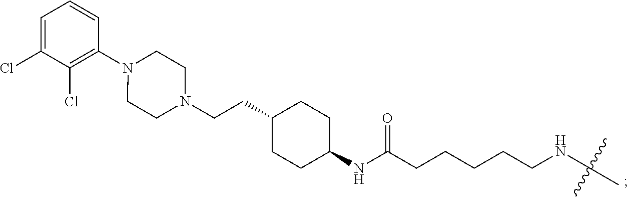 [SMILES: *NCCCCCC(=O)N[C@H]1CC[C@H](CCN2CCN(c3cccc(Cl)c3Cl)CC2)CC1]

L008: linker having the structure of —C(O)—(CH₂)₉—, wherein —C(O)— is connected to Mod (through —NH—) or —OH (if no Mod indicated), and the —CH₂— connecting site is connected to an oligonucleotide chain (e.g., at the 5′-end) through a linkage, e.g., phosphodiester (—O—P(O)(OH)—O—, which may exist as a salt form, and may be indicated as O or PO), phosphorothioate (—O—P(O)(SH)—O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration), or phosphorodithioate (—O—P(S)(SH)—O—, which may exist as a salt form, and may be indicated as PS2 or: or D) linkage. For example, in WV-11571, L008 is connected to —OH through —C(O)—, and the 5′-end of an oligonucleotide chain through a phosphate linkage (indicated as “O” in “Stereochemistry/Linkage”); in WV-11569, L008 is connected to Mod062 through —C(O)—, and the 5′-end of an oligonucleotide chain through a phosphate linkage (indicated as “O” in “Stereochemistry/Linkage”);
Mod001 (with —C(O)— connecting to —NH— of a linker such as L001):

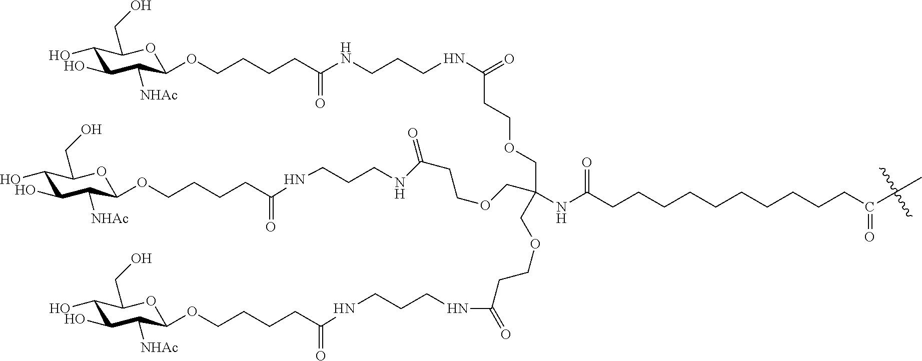

Mod085 (with —C(O)— connecting to —NH— of a linker such as L001 or L004):

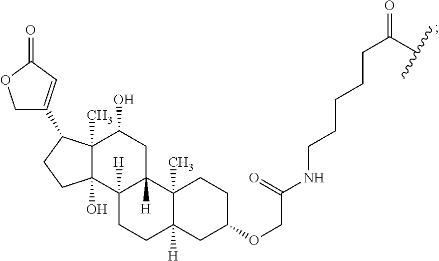

Mod086 (with —C(O)— connecting to —NH— of a linker such as L001 or L004):

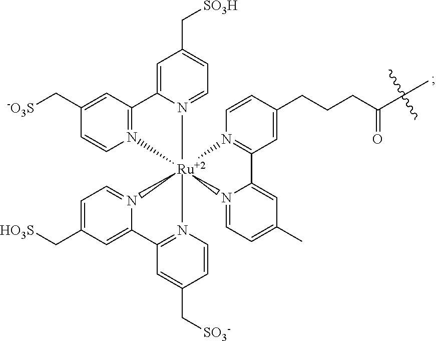

Mod094 (in WV-11570, bound to the 3′-end of the oligonucleotide chain (3′-carbon of the 3′-end sugar) through a phosphate group (which is not shown below and which may exist as a salt form; and which is indicated as “O” in “Stereochemistry/Linkage” ( . . . XXXXO))):

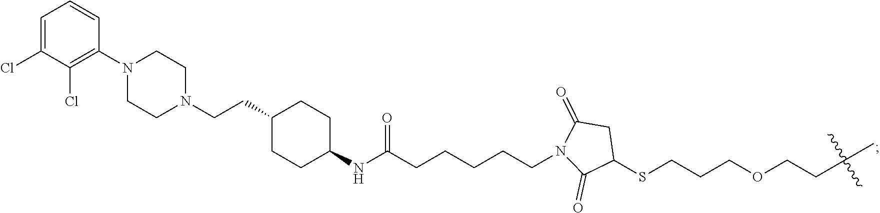 [SMILES: *CCOCCCSC1CC(=O)N(CCCCCC(=O)N[C@H]2CC[C@H](CCN3CCN(c4cccc(Cl)c4Cl)CC3)CC2)C1=O]

BrdU: a nucleoside unit wherein the nucleobase is BrU

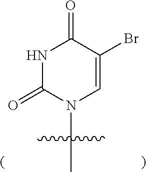 [SMILES: *n1cc(Br)c(=O)[nH]c1=O]

and wherein the sugar is 2-deoxyribose (as widely found in natural DNA; 2′-deoxy (d))

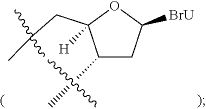

tgal mc6T: modified thymidine comprising a modified thymine and having the structure of:

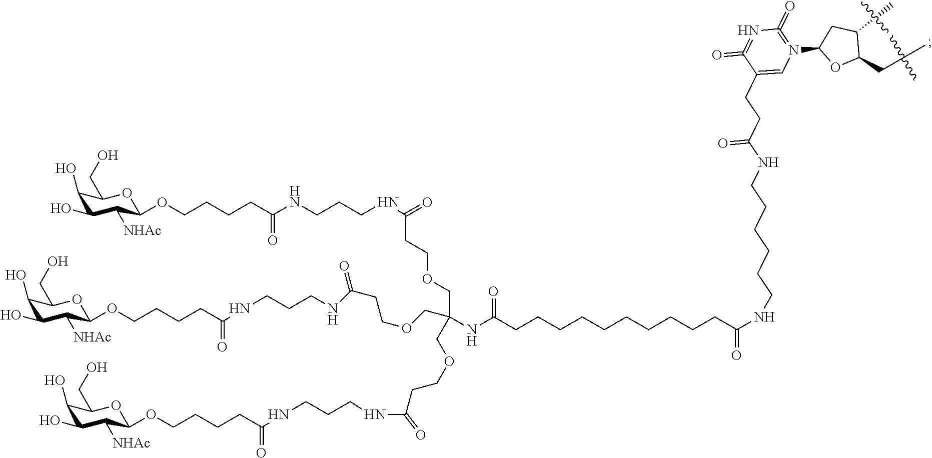

d2AP: a nucleoside unit wherein the nucleobase is 2-amino purine

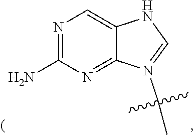

2AP) and wherein the sugar is 2-deoxyribose (as widely found in natural DNA; 2′-deoxy (d))

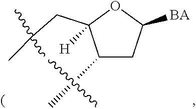

### BA=2AP);

dDAP: a nucleoside unit wherein the nucleobase is 2,6-diamino purine

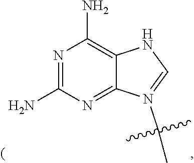

DAP) and wherein the sugar is 2-deoxyribose (as widely found in natural DNA; 2′-deoxy (d))

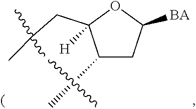

### BA=DAP);

dmtr: DMTR, 4,4′-dimethoxytrityl, bonded to 5′-O— of a sugar unless indicated otherwise. For example, in dmtrmA:

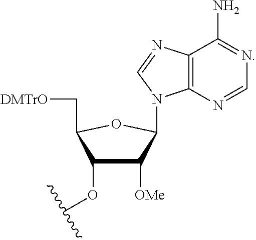

Additional structural elements of HTT oligonucleotides are described in, for example: WO 2018/022473, WO 2018/098264, WO 2018/223056, WO 2018/223073, WO 2018/223081, WO 2018/237194, WO 2019/032607, WO 2019/055951, and/or WO 2019/075357, the structural elements of oligonucleotides of which are hereby incorporated by reference.

### Lengths

As appreciated by those skilled in the art, oligonucleotides can be of various lengths to provide desired properties and/or activities for various uses. Many technologies for assessing, selecting and/or optimizing oligonucleotide length are available in the art and can be utilized in accordance with the present disclosure. As demonstrated herein, in many embodiments, provided oligonucleotides are of suitable lengths to hybridize with their targets and reduce levels of their targets and/or an encoded product thereof. In some embodiments, an oligonucleotide is long enough to recognize a target HTT nucleic acid (e.g., an HTT mRNA). In some embodiments, an oligonucleotide is sufficiently long to distinguish between a target HTT nucleic acid and other nucleic acids (e.g., a nucleic acid having a base sequence which is not HTT) to reduce off-target effects. In some embodiments, an oligonucleotide, e.g., an HTT oligonucleotide, is sufficiently short to reduce complexity of manufacture or production and to reduce cost of products.

In some embodiments, the base sequence of an oligonucleotide is about 10-500 nucleobases in length. In some embodiments, a base sequence is about 10-500 nucleobases in length. In some embodiments, a base sequence is about 10-50 nucleobases in length. In some embodiments, a base sequence is about 15-50 nucleobases in length. In some embodiments, a base sequence is from about 15 to about 30 nucleobases in length. In some embodiments, a base sequence is from about 10 to about 25 nucleobases in length. In some embodiments, a base sequence is from about 15 to about 22 nucleobases in length. In some embodiments, a base sequence is about 10, 11, 12, 13, 14, 15, 16, 17, 18, 19, 20, 21, 22, 23, 24, or 25 nucleobases in length.

In some embodiments, each nucleobase independently comprises an optionally substituted monocyclic, bicyclic or polycyclic ring wherein at least one ring atom is nitrogen. In some embodiments, each nucleobase is independently optionally substituted adenine, cytosine, guanosine, thymine, or uracil, or an optionally substituted tautomer of adenine, cytosine, guanosine, thymine, or uracil.

### Regions, Wings and Cores of HTT Oligonucleotides

In some embodiments, an oligonucleotide, e.g., an HTT oligonucleotide, comprises several regions, each of which independently comprises one or more consecutive nucleosides and optionally one or more internucleotidic linkages. In some embodiments, a region differs from its neighboring region(s) in that it contains one or more structural feature that are different from those corresponding structural features of its neighboring region(s). Example structural features include nucleobase modifications and patterns thereof, sugar modifications and patterns thereof, internucleotidic linkages and patterns thereof (which can be internucleotidic linkage types (e.g., phosphate, phosphorothioate, phosphorothioate triester, neutral internucleotidic linkage, etc.) and patterns thereof, linkage phosphorus modifications (backbone phosphorus modifications) and patterns thereof (e.g., pattern of —XLR¹ if internucleotidic linkages having the structure of formula I), backbone chiral center (linkage phosphorus) stereochemistry and patterns thereof [e.g., combination of Rp and/or Sp of chirally controlled internucleotidic linkages (sequentially from 5′ to 3′), optionally with non-chirally controlled internucleotidic linkages and/or natural phosphate linkages, if any (e.g., OSOOO RSSRS SSSRS SOOOS in Table 1)]. In some embodiments, a region comprises a chemical modification (e.g., a sugar modification, base modification, internucleotidic linkage, or stereochemistry of internucleotidic linkage) not present in its neighboring region(s). In some embodiments, a region lacks a chemical modification present in its neighboring regions(s).

In some embodiments, an oligonucleotide, e.g., an HTT oligonucleotide, comprises or consists of two or more regions. In some embodiments, an oligonucleotide comprises or consists of three or more regions. In some embodiments, an oligonucleotide comprises or consists of two neighboring regions, wherein one region is designated as a wing region and the other a core region. The structure of such an oligonucleotide comprises or consists of a wing-core or core-wing structure. In some embodiments, an oligonucleotide comprises or consists of three neighboring regions, wherein one region is flanked by two neighboring regions. In some embodiments, the middle region is designated as the core region, and each of the flanking region a wing region (a 5′-wing if connected to the 5′-end of the core, a 3′-wing if connected to the 3′-end of the core). The structure of such an oligonucleotide comprises or consists of a wing-core-wing structure.

In some embodiments, a first region (e.g., a wing) differs from a second region (e.g., a core) in that the first region contains sugar modification(s) or pattern thereof absent from the second region. In some embodiments, a first (e.g., wing) region comprises a sugar modification absent from a second (e.g., core) region. In some embodiments, a sugar modification is a 2′-modification. In some embodiments, a 2′-modification is 2′-OR, wherein R is optionally substituted C₁₋₆ aliphatic. In some embodiments, a 2′-modification is 2′-OR, wherein R is optionally substituted C₁₋₆ alkyl. In some embodiments, a 2′-modification is 2′-MOE. In some embodiments, a 2′-modification is 2′-OMe. In some embodiments, a modified sugar is a bicyclic sugar, e.g., a LNA sugar. In some embodiments, each sugar in a region is independently modified. In some embodiments, each sugar of a region (e.g., a wing) independently comprises a modification, which can be the same or different from each other. In some embodiments, each sugar of a region (e.g., a wing) comprises the same modification, e.g., 2′-modification as described in the present disclosure. In some embodiments, sugars of a region (e.g., a core) are not modified. In some embodiments, each sugar of a region (e.g., a core) is a non-modified DNA sugar (with two —H at the 2′-position). In some embodiments, the structure of a provided oligonucleotide comprises or consists of a wing-core, core-wing, or wing-core-wing structure, wherein each wing independently comprises one or more sugar modifications, and each sugar in the core is a natural DNA sugar (with two —H at the 2′-position).

Additionally or alternatively, a first region (e.g., a wing) can contain internucleotidic linkage(s) or pattern thereof that differs from another region (e.g., a core or another wing). In some embodiments, a region (e.g., a wing) comprises two or more consecutive natural phosphate linkages. In some embodiments, a region (e.g., a core) comprises no consecutive natural phosphate linkages. In some embodiments, the structure of a provided oligonucleotide comprises or consists of a wing-core, core-wing, or wing-core-wing structure, wherein at least one wing independently comprises two or more consecutive natural phosphate linkages, and the core comprises no consecutive natural phosphate linkages. In some embodiments, in a wing-core-wing structure, each wing independently comprises two or more consecutive internucleotidic linkages. Unless otherwise noted, for the purpose of stereochemistry of wing-core-wing structures, internucleotidic linkages connecting a core with a wing are included in the core (e.g., see above).

In some embodiments, a region is a 5′-wing, a 3′-wing, or a core. In some embodiments, the 5′-wing is to the 5′ end of the oligonucleotide, the 3′-wing is to the 3′-end of the oligonucleotide and the core is between the 5′-wing and the 3′-wing, and the oligonucleotide comprises or consists of a wing-core-wing structure or format. In some embodiments, a core comprises a span of contiguous natural DNA sugars (2′-deoxyribose). In some embodiments, a core comprises a span of at least 5 contiguous natural DNA sugars (2′-deoxyribose). In some embodiments, a core comprises a span of at least 10 contiguous natural DNA sugars (2′-deoxyribose). In some embodiments, a core is referenced as a gap. In some embodiments, an oligonucleotide which comprises or consists of a wing-core-wing structure is described as a gapmer. In some embodiments, the structure of a provided oligonucleotide comprises or consists of a wing-core structure. In some embodiments, the structure of a provided oligonucleotide comprises or consists of a core-wing structure. Non-limiting examples of oligonucleotides having a core-wing structure include WV-2023 and WV-2025. In some embodiments, the structure of an oligonucleotide comprises or consists of an oligonucleotide chain which comprises or consists of wing-core-wing, wing-core, or wing-core, wherein the oligonucleotide chain is conjugated to an additional chemical moiety optionally through a linker as described in the present disclosure. In some embodiments, the present disclosure provides oligonucleotides that target HTT and have a structure that comprises one or two wings and a core, and comprise or consist of a wing-core-wing, core-wing, or wing-core structure.

Ribonuclease H (RNase H, e.g., RNase H1, RNase H2, etc.) reportedly recognizes a structure comprising a hybrid of RNA and DNA (e.g., a heteroduplex), and cleaves the RNA. In some embodiments, an oligonucleotide comprising a span of contiguous natural DNA sugars (2′-deoxyribose, e.g., in a core region) is capable of annealing to a RNA such as a mRNA to form a heteroduplex; and this heteroduplex structure is capable of being recognized by RNase H and the RNA cleaved by RNase H. In some embodiments, a core of a provide oligonucleotide comprises 1, 2, 3, 4, 5, 6, 7, 8, 9, 10 or more contiguous natural DNA sugars, and the core is capable of annealing specifically to a target transcript [e.g., an HTT transcript (e.g., pre-mRNA, mature mRNA, etc.)]; and the formed structure is capable of being recognized by RNase H and the transcript cleaved by RNase H. In some embodiments, a core of a provided oligonucleotide comprises 5 or more contiguous DNA sugars.

Regions, e.g., wings, cores, etc., can be of various suitable lengths. In some embodiments, a region (e.g., a wing, a core, etc.) contains 1, 2, 3, 4, 5, 6, 7, 8, 9, 10, 11, 12, 13, 14, 15, 16, 17, 18, 19, 20, 21, 22, 23, 24, 25 or more nucleobases. As described in the present disclosure, in some embodiments, each nucleobase independently comprises an optionally substituted monocyclic, bicyclic or polycyclic ring, which ring has at least one nitrogen ring atom; in some embodiments, each nucleobase is independently optionally substituted A, T, C, G or U, or a substituted tautomer of A, T, C, G or U. In some embodiments, the number is 1, 2, 3, 4, 5, 6, 7, 8, 9, or 10 for a wing. In some embodiments, each wing of a wing-core-wing structure independently has a length as described in the present disclosure. In some embodiments, the two wings are of the same length. In some embodiments, the two wings are of different length. In some embodiments, the number is 1, 2, 3, 4, 5, 6, 7, 8, 9, 10, 11, 12, 13, 14, 15 or more for a core.

In some embodiments, a wing comprises one or more sugar modifications. In some embodiments, the two wings of a wing-core-wing structure comprise different sugar modifications (and the oligonucleotide has or comprises an “asymmetric” format). In some embodiments, sugar modifications provide improved stability and/or annealing properties compared to absence of sugar modifications.

In some embodiments, certain sugar modifications, e.g., 2′-MOE, provide more stability under certain conditions than other sugar modifications, e.g., 2′-OMe. In some embodiments, a wing comprises 2′-MOE modifications. In some embodiments, each nucleoside unit of a wing comprising a pyrimidine base (e.g., C, U, T, etc.) comprises a 2′-MOE modification. In some embodiments, each sugar unit of a wing comprises a 2′-MOE modification. In some embodiments, each nucleoside unit of a wing comprising a purine base (e.g., A, G, etc.) comprises no 2′-MOE modification (e.g., each such nucleoside unit comprises 2′-OMe, or no 2′-modification, etc.). In some embodiments, each nucleoside unit of a wing comprising a purine base comprises a 2′-OMe modification. In some embodiments, each internucleotidic linkage at the 3′-position of a sugar unit comprising a 2′-MOE modification is a natural phosphate linkage.

In some embodiments, a wing comprises no 2′-MOE modifications. In some embodiments, a wing comprises 2′-OMe modifications. In some embodiments, each nucleoside unit of a wing independently comprises a 2′-OMe modification.

In some embodiments, the structure of an oligonucleotide, e.g., an HTT oligonucleotide, comprises a wing-core-wing structure, wherein one wing comprises a 2′-OMe sugar modification and the other wing comprises a bicyclic sugar; wherein one wing comprises 2′-OMe and the other wing comprises a bicyclic sugar, and the majority of the sugars in the core are natural DNA sugars (with no substitution at the 2′-position); wherein the majority of the sugars in one wing comprise 2′-OMe and the majority of the sugars in the other wing are independently bicyclic sugars; wherein the majority of the sugars in one wing comprise 2′-OMe and the majority of the sugars in the other wing are independently bicyclic sugars, and the majority of the sugars in the core are natural DNA sugars; wherein the majority of the sugars in one wing comprise 2′-OMe and, in the other wing, at least one sugar is a bicyclic sugar and at least one sugar comprises 2′-OMe; wherein the majority of the sugars in one wing comprise 2′-OMe and, in the other wing, at least one sugar is a bicyclic sugar and at least one sugar comprises 2′-OMe, and the majority of the sugars in the core are natural DNA sugars; wherein the majority of the sugars in one wing are bicyclic sugars and, in the other wing, at least one sugar is a bicyclic sugar and at least one sugar comprises 2′-OMe; wherein the majority of the sugars in one wing are independently bicyclic sugars and, in the other wing, at least one sugar is a bicyclic sugar and at least one sugar comprises 2′-OMe, and the majority of the sugars in the core are natural DNA sugars; wherein each sugar in one wing comprises 2′-OMe and each sugar in the other wing is independently a bicyclic sugar; wherein each sugar in one wing comprises 2′-OMe and each sugar in the other wing is independently a bicyclic sugar, and the majority of the sugars in the core are natural DNA sugars; wherein each sugar in one wing is independently a bicyclic sugar, each sugar in the other wing comprises 2′-OMe, and each sugar in the core is a natural DNA sugar; wherein one wing comprises a bicyclic sugar and the other wing comprises 2′-MOE; wherein one wing comprises a bicyclic sugar and the other wing comprises 2′-MOE, and the majority of the sugars in the core are natural DNA sugars; wherein the majority of the sugars in one wing are independently bicyclic sugars and the majority of the sugars in the other wing comprise 2′-MOE; wherein the majority of the sugars in one wing comprise are independently bicyclic sugars and the majority of the sugars in the other wing comprise 2′-MOE, and the majority of the sugars in the core are natural DNA sugars; wherein the majority of the sugars in one wing are independently bicyclic sugars and, in the other wing, at least one sugar comprises 2′-MOE and at least one sugar is a bicyclic sugar; wherein the majority of the sugars in one wing are independently bicyclic sugars and, in the other wing, at least one sugar comprises 2′-MOE and at least one sugar is a bicyclic sugar, and the majority of the sugars in the core are natural DNA sugars; wherein the majority of the sugars in one wing comprise 2′-MOE and, in the other wing, at least one sugar comprises 2′-MOE and at least one sugar is a bicyclic sugar; wherein the majority of the sugars in one wing comprise 2′-MOE and, in the other wing, at least one sugar comprises 2′-MOE and at least one sugar is a bicyclic sugar, and the majority of the sugars in the core are natural DNA sugars; wherein each sugar in one wing is independently a bicyclic sugar and each sugar in the other wing independently comprises 2′-MOE; and/or wherein each sugar in one wing is independently a bicyclic sugar and each sugar in the other wing of the oligonucleotide comprises 2′-MOE, and the majority of the sugars in the core are natural DNA sugars.

In some embodiments, the structure of an oligonucleotide, e.g., an HTT oligonucleotide, comprises a wing-core-wing structure, wherein each sugar in one wing comprises 2′-MOE, each sugar in the other wing is independently a bicyclic sugar, and each sugar in the core is a natural DNA sugar.

In some embodiments, a bicyclic sugar is a LNA, a cEt or a BNA sugar.

In some embodiments, the structure of an oligonucleotide, e.g., an HTT oligonucleotide, comprises a wing-core-wing structure, wherein one wing comprises 2′-OMe and the other wing comprises 2′-F. In some embodiments, the structure of an oligonucleotide, e.g., an HTT oligonucleotide, comprises a wing-core-wing structure, wherein one wing comprises 2′-OMe and the other wing comprises 2′-F, and the majority of the sugars in the core are natural DNA sugars.

In some embodiments, the structure of an oligonucleotide, e.g., an HTT oligonucleotide, comprises a wing-core-wing structure, wherein the majority of the sugars in one wing comprise 2′-OMe and the majority of the sugars in the other wing comprise 2′-F. In some embodiments, the structure of an oligonucleotide, e.g., an HTT oligonucleotide, comprises a wing-core-wing structure, wherein the majority of the sugars in one wing comprise 2′-OMe and the majority of the sugars in the other wing comprise 2′-F, and the majority of the sugars in the core are natural DNA sugars.

In some embodiments, the structure of an oligonucleotide, e.g., an HTT oligonucleotide, comprises a wing-core-wing structure, wherein the majority of the sugars in one wing comprise 2′-OMe and, in the other wing, at least one sugar comprises 2′-F and at least one sugar comprises 2′-OMe. In some embodiments, the structure of an oligonucleotide, e.g., an HTT oligonucleotide, comprises a wing-core-wing structure, wherein the majority of the sugars in one wing comprise 2′-OMe and, in the other wing, at least one sugar is 2′-F and at least one sugar comprises 2′-OMe, and the majority of the sugars in the core are DNA sugars.

In some embodiments, the structure of an oligonucleotide, e.g., an HTT oligonucleotide, comprises a wing-core-wing structure, wherein the majority of the sugars in one wing comprise 2′-F and, in the other wing, at least two sugars comprise 2′-F and at least two sugars comprise 2′-OMe. In some embodiments, the structure of an oligonucleotide, e.g., an HTT oligonucleotide, comprises a wing-core-wing structure, wherein the majority of the sugars in one wing comprise 2′-F and, in the other wing, at least two sugars comprise 2′-F and at least two sugars comprise 2′-OMe, and the majority of the sugars in the core are natural DNA sugars.

In some embodiments, the structure of an oligonucleotide, e.g., an HTT oligonucleotide, comprises a wing-core-wing structure, wherein each sugar in one wing of the oligonucleotide comprises 2′-OMe and each sugar in the other wing of the provided oligonucleotide comprises 2′-F. In some embodiments, the structure of an oligonucleotide, e.g., an HTT oligonucleotide, comprises a wing-core-wing structure, wherein each sugar in one wing of the oligonucleotide comprises 2′-OMe and each sugar in the other wing of the oligonucleotide comprises 2′-F, and the majority of the sugars in the core are natural DNA sugars.

In some embodiments, the structure of an oligonucleotide, e.g., an HTT oligonucleotide, comprises a wing-core-wing structure, wherein each sugar in one wing comprises 2′-F, each sugar in the other wing comprises 2′-OMe, and each sugar in the core is a DNA sugar.

In some embodiments, the structure of an oligonucleotide, e.g., an HTT oligonucleotide, comprises a wing-core-wing structure, wherein one wing comprises 2′-F and the other wing comprises 2′-MOE. In some embodiments, the structure of an oligonucleotide, e.g., an HTT oligonucleotide, comprises a wing-core-wing structure, wherein one wing comprises 2′-F and the other wing comprises 2′-MOE, and the majority of the sugars in the core comprise 2′-deoxy.

In some embodiments, the structure of an oligonucleotide, e.g., an HTT oligonucleotide, comprises a wing-core-wing structure, wherein the majority of the sugars in one wing comprise 2′-F and the majority of the sugars in the other wing comprise 2′-MOE. In some embodiments, the structure of an oligonucleotide, e.g., an HTT oligonucleotide, comprises a wing-core-wing structure, wherein the majority of the sugars in one wing comprise 2′-F and the majority of the sugars in the other wing comprise 2′-MOE, and the majority of the sugars in the core are natural DNA sugars.

In some embodiments, the structure of an oligonucleotide, e.g., an HTT oligonucleotide, comprises a wing-core-wing structure, wherein the majority of the sugars in one wing comprise 2′-F and, in the other wing, at least one sugar comprises 2′-MOE and at least one sugar comprises 2′-F. In some embodiments, the structure of an oligonucleotide, e.g., an HTT oligonucleotide, comprises a wing-core-wing structure, wherein the majority of the sugars in one wing comprise 2′-F and, in the other wing, at least one sugar comprises 2′-MOE and at least one sugar comprises 2′-F, and the majority of the sugars in the core are natural DNA sugars.

In some embodiments, the structure of an oligonucleotide, e.g., an HTT oligonucleotide, comprises a wing-core-wing structure, wherein the majority of the sugars in one wing comprise 2′-MOE and, in the other wing, at least one sugar comprises 2′-MOE and at least one sugar comprises 2′-F. In some embodiments, the structure of an oligonucleotide, e.g., an HTT oligonucleotide, comprises a wing-core-wing structure, wherein the majority of the sugars in one wing comprise 2′-MOE and, in the other wing, at least one sugar comprises 2′-MOE and at least one sugar comprises 2′-F, and the majority of the sugars in the core are natural DNA sugars.

In some embodiments, the structure of an oligonucleotide, e.g., an HTT oligonucleotide, comprises a wing-core-wing structure, wherein each sugar in one wing of the oligonucleotide comprises 2′-MOE, each sugar in the other wing comprises 2′-F, and each sugar in the core are natural DNA sugars.

In some embodiments, an oligonucleotide, e.g., an HTT oligonucleotide, has a wing-core-wing structure. In some embodiments, a core comprises 1 or more natural DNA sugars. In some embodiments, a core comprises 5 or more consecutive natural DNA sugars. In some embodiments, the core comprises 5-10, 5-15, 5-20, 5-25, 5-30, or 5, 6, 7, 8, 9, 10, 11, 12, 13, 14, 15 or more natural DNA sugars which are optionally consecutive. In some embodiments, the core comprises 5, 6, 7, 8, 9, 10, 11, 12, 13, 14, 15 or more consecutive natural DNA sugars. In some embodiments, core comprises 10 or more consecutive natural DNA sugars. In some embodiments, the core is able to hybridize to a target mRNA, forming a duplex structure recognizable by RNaseH, such that RNaseH is able to cleave the mRNA.

In some embodiments, an oligonucleotide, e.g., an HTT oligonucleotide, has a wing-core-wing structure and has an asymmetrical format.

In some embodiments, in an oligonucleotide having an asymmetrical format, one wing differs from another in the sugar modifications or pattern thereof, or the backbone internucleotidic linkages or pattern thereof, or the backbone chiral centers or pattern thereof. In some embodiments, an oligonucleotide, e.g., an HTT oligonucleotide, has an asymmetrical format in that one wing comprises a different sugar modification than the other wing. In some embodiments, an oligonucleotide, e.g., an HTT oligonucleotide, has an asymmetrical format in that one wing comprises a different pattern of sugar modifications than the other wing.

In some embodiments, a HTT oligonucleotide (or a wing, core, block or any portion thereof) can comprise any modification, any pattern of modifications, any internucleotidic linkage, any pattern of internucleotidic linkages, any pattern of chiral centers, or any format (including but not limited to an asymmetrical format) described in any of: WO2017015555; WO2017192664; WO0201200366; WO2011/034072; WO2014/010718; WO2015/108046; WO2015/108047; WO2015/108048; WO 2011/005761; WO 2011/108682; WO 2012/039448; WO 2018/067973; WO2005/028494; WO2005/092909; WO2010/064146; WO2012/073857; WO2013/012758; WO2014/010250; WO2014/012081; WO2015/107425; WO2017/015555; WO2017/015575; WO2017/062862; WO2017/160741; WO2017/192664; WO2017/192679; WO2017/210647; WO2018/022473; or WO2018/098264, of which each modification, any pattern of modifications, any internucleotidic linkage, any pattern of internucleotidic linkages, or any format (including but not limited to an asymmetrical format) described therein is incorporated by reference.

In some embodiments, the structure of an oligonucleotide, e.g., an HTT oligonucleotide, comprises or consists of an asymmetrical format. In some embodiments, the structure of an oligonucleotide, e.g., an HTT oligonucleotide, comprises or consists of a symmetrical format.

In some embodiments, the structure of an oligonucleotide, e.g., an HTT oligonucleotide, is or comprises an asymmetrical format, wherein the structure of the oligonucleotide is a wing-core-wing structure, wherein the format of the first wing is different from that of the second wing. In some embodiments, the structure of an oligonucleotide, e.g., an HTT oligonucleotide, is or comprises an asymmetrical format, wherein the structure of the oligonucleotide is a wing-core-wing structure, wherein the first and second wings differ in sugar modification (or combinations or patterns thereof) and/or in internucleotidic linkages (or combinations or patterns thereof). In some embodiments, the structure of an oligonucleotide, e.g., an HTT oligonucleotide, is or comprises an asymmetrical format, wherein the structure of the oligonucleotide is a wing-core-wing structure, wherein the first and second wings differ in sugar modification (or combinations or patterns thereof).

In some embodiments, a core region comprises a sequence complementary to one allele of a differentiating position, e.g., a SNP location. In some embodiments, a core region comprises a sequence complementary to one allele of a SNP (e.g., which is on the same strand/chromosome as a disease-associated or causing sequence (e.g., expanded CAG repeats in an HTT gene)) but is not complementary to other alleles of a SNP (e.g., which is on the same strand/chromosome as a less or non-disease-associated or causing sequence (e.g., normal or shorter CAG repeats in an HTT gene)). In some embodiments, for SNP such a sequence is one nucleobase. In some embodiments, a core region comprises a nucleobase complementary to an allele of a SNP which is on the same strand/chromosome as expanded CAG repeats in an HTT gene. Among other things, the present disclosure demonstrates that properties and/or activities of oligonucleotides may be modulated through positioning of such a nucleobase. In some embodiments, a position of such a nucleobase is position 4, 5, 6, 7 or 8 counting from the 5′-end of a core region (the first nucleoside of the core region from the 5′-end being position 1). In some embodiments, a position is position 4 from the 5′-end of a core region. In some embodiments, a position is position 5 from the 5′-end of a core region. In some embodiments, a position is position 6 from the 5′-end of a core region. In some embodiments, a position is position 7 from the 5′-end of a core region. In some embodiments, a position is position 8 from the 5′-end of a core region. In some embodiments, a position of such a nucleobase is position 7, 8, 9, 10, 11 or 12 counting from the 5′-end of an oligonucleotide (the first nucleoside of the oligonucleotide from the 5′-end being position 1). In some embodiments, a position is position 7 from the 5′-end of an oligonucleotide. In some embodiments, a position is position 8 from the 5′-end of an oligonucleotide. In some embodiments, a position is position 9 from the 5′-end of an oligonucleotide. In some embodiments, a position is position 10 from the 5′-end of an oligonucleotide. In some embodiments, a position is position 11 from the 5′-end of an oligonucleotide. In some embodiments, an oligonucleotide comprises a 5′-end wing comprising 5 and no more than 5 nucleosides. In some embodiments, each wing sugar is 2′-modified. In some embodiments, each wing sugar is 2′-OMe modified. In some embodiments, each core sugar independently comprises no 2′-OR modification, wherein R is as described in the present disclosure. In some embodiments, each core sugar is independently an unmodified DNA sugar.

In some embodiments, an oligonucleotide, e.g., an HTT oligonucleotide, may comprise any first wing, core and/or second wing, as described herein or known in the art.

In some embodiments, an oligonucleotide which has a base sequence which is, comprises or comprises a span of an HTT oligonucleotide sequence disclosed herein can comprise a first wing, core and/or second wing, as described herein or known in the art.

### RNAi Agents

Oligonucleotides of the present disclosure can perform one or more functions through various biological mechanisms and/or pathways. In some embodiments, the present disclosure provides oligonucleotide that can reduce levels, expression and/or activities of genes or products thereof partially, mainly or wholly through RNA interference. As appreciated by those skilled in the art, such oligonucleotides can be either single- or double-stranded. In some embodiments, a single- or double-stranded oligonucleotide is capable of decreasing the level, expression and/or activity of a target gene (e.g., HTT) or a gene product thereof, via a mechanism involving RNA interference.

In some embodiments, the present disclosure pertains to an oligonucleotide, e.g., an HTT oligonucleotide, which has a base sequence which comprises, is or comprises a span of 15 contiguous bases or more (with, optionally, 1-3 mismatches) from the base sequence of an oligonucleotide in Table 1, wherein the oligonucleotide is capable of mediating RNA interference.

In some embodiments, the present disclosure pertains to an HTT oligonucleotide which has a base sequence which comprises, is or comprises a span of 15 contiguous bases or more (with, optionally, 1-3 mismatches) from the base sequence of an oligonucleotide in Table 1, wherein the HTT oligonucleotide is capable of mediating single-stranded RNA interference.

In some embodiments, the present disclosure pertains to an HTT oligonucleotide which has a base sequence which comprises, is or comprises a span of 15 contiguous bases or more (with, optionally, 1-3 mismatches) from the base sequence of an oligonucleotide in Table 1, wherein the HTT oligonucleotide is capable of mediating single-stranded RNA interference.

In some embodiments, a RNAi agent is an agent (e.g., a nucleic acid, including but not limited to a single- or double-stranded nucleic acid) which is capable of mediating RNA interference. In some embodiments, the present disclosure provides RNAi agent that targets HTT.

In some embodiments, the present disclosure pertains to a single-stranded RNAi agent whose base sequence is or comprises a sequence that is or is complementary to a span of 15-30 (e.g., at least 15, 16, 17, 18, 19, 20 or 21) contiguous bases of HTT or a transcripts thereof. In some embodiments, the present disclosure pertains to a single-stranded RNAi agent which has a base sequence which is or comprises or comprises a span of at least 15 contiguous bases of any HTT oligonucleotide in Table 1. In some embodiments, such a span of contiguous bases is characteristic of HTT and it is not identical or complementary to any other sequences in a genome or transcriptome.

In some embodiments, the present disclosure pertains to a double-stranded RNAi agent comprising a sense and an antisense strand, wherein the base sequence of the antisense strand is or comprises a sequence that is or is complementary to a span of 15-30 (e.g., at least 15, 16, 17, 18, 19, 20 or 21) contiguous bases of HTT or a transcripts thereof. In some embodiments, the present disclosure pertains to a double-stranded RNAi agent comprising a sense and an antisense strand, wherein the antisense strand has a base sequence which is or comprises or comprises a span of at least 15 contiguous bases of any HTT oligonucleotide in Table 1. In some embodiments, the present disclosure pertains to a double-stranded RNAi agent comprising a sense and an antisense strand, wherein the antisense strand has a base sequence which is or comprises or comprises a span of at least 10 contiguous bases of any HTT oligonucleotide in Table 1. In some embodiments, such a span of contiguous bases is characteristic of HTT and it is not identical or complementary to any other sequences in a genome or transcriptome.

In some embodiments, an RNAi agent, e.g., an HTT RNAi agent, can be the format of a RNAi agent, whether double- or single-stranded, described herein or known in the art. Various formats of double-stranded RNAi agents are described in the art and may be utilized in accordance with the present disclosure, for example, in: Elbashir et al. 2001 Gen. Dev. 15: 188; Elbashir et al. 2001 Nature 411: 494; Elbashir et al. 2001 EMBO J. 20: 6877-6888; Sun et al. Nat. Biotech. 26: 1379; Chiu et al. 2003 RNA 9: 1034-1048; Kim et al. (2005) Nat Biotech 23:222-226; U.S. Pat. Nos. 8,084,600; 9,175,289; 8,329,888; 8,090,542; 7,507,811; 8,828,956; US 20130035368; US 20050255487; US 20080242851; WO 2015051366; and EP 3052464. Various formats of single-stranded RNAi agents are described in the art and may be utilized in accordance with the present disclosure, for example, in: EP1520022, U.S. Pat. Nos. 8,729,036, 9,476,044, 9,243,246, WO 2004/007718, etc.

In some embodiments, the strand of a single-stranded RNAi agent or the antisense strand of a double-stranded RNAi agent comprises, in order, from 5′ to 3′, a 5′-end region, a seed region, a post-seed region, and a 3′ end. In some embodiments, in the strand, a seed region comprises the nucleotides at positions about 2 to about 7 or about 8, counting from the 5′ end. In some embodiments, the 5′-end region comprises the portion of the strand 5′ to the seed region. In some embodiments, the 3′-end region comprises either a terminal dinucleotide (e.g., TT or UU) at the 3′ end, or a moiety (e.g., a 3′ end cap) which functionally replaces the terminal dinucleotide. 3′ end caps are described in, for example: U.S. Pat. No. 8,084,600 and WO 2015/051366. In some embodiments, the post-seed region comprises the portion of the strand between the seed region and the 3′ end region.

In some embodiments, the 5′ end region comprises a phosphate group or an analog thereof. In some embodiments, conjugated, e.g., directly or indirectly to the 5′ end region, is an additional chemical moiety as described herein. In some embodiments, conjugated, e.g., directly or indirectly to the 5′ end region, is an additional chemical moiety which is a GalNAc or derivative thereof capable of binding to ASPGR.

In some embodiments, the seed region is particularly important for recognizing and complementing the target region. In some embodiments, the seed region is less suitable for mismatches to the target than the 5′ end region or the post-seed region.

In some embodiments, a single-stranded RNAi agent, e.g., a single-stranded HTT RNAi reagent, comprises a chemical moiety at the 5′ end comprising phosphorus. In some embodiments, a single-stranded RNAi agent has a group comprising phosphorus at its 5′-end. In some embodiments, a single-stranded RNAi agent has a phosphate group or an analog thereof at its 5′-end.

In some embodiments, bound to a single-stranded RNAi agent, or to either or both strands of a double-stranded RNAi agent is a ASPGR ligand. In some embodiments, a ASGPR ligand is GalNAc or a derivative thereof that is capable of binding to ASPGR.

Non-limiting examples of oligonucleotides that may be utilized as single-stranded RNAi agents include: WV-5153, WV-5154, WV-5155, WV-5156, WV-5157, WV-5158, WV-5159, WV-5160, WV-5161, WV-5162, WV-5163, WV-5164, WV-5165, WV-5166, WV-5167, WV-5168, WV-5169, WV-5170, WV-5171, WV-5172, WV-5173, WV-5174, WV-5175, WV-5176, WV-5177, WV-5178, WV-5179, WV-5180, WV-5181, WV-5182, WV-5183, WV-5184, WV-5185, WV-5186, WV-5187, WV-5188, WV-5189, WV-5190, WV-5191, WV-5192, WV-5193, WV-5194, WV-5195, WV-5196, WV-5197, WV-5198, WV-5199, WV-5200, WV-5201, WV-5202, WV-5203, WV-5204, WV-5205, WV-5206, WV-5207, WV-5208, WV-5209, WV-5210, WV-5211, WV-5212, WV-5213, WV-5214, WV-5215, WV-5216, WV-5217, WV-5218, WV-5219, WV-5220, WV-5221, WV-5222, WV-5223, WV-5224, WV-5225, WV-5226, WV-5227, WV-5228, WV-5229, WV-5230, WV-5231, WV-5232, WV-5233, WV-5234, WV-5235, WV-5236, WV-5237, WV-5238, WV-5239, WV-5240, WV-5241, WV-5242, WV-5243, WV-5244, WV-5245, WV-5246, WV-5247, WV-5248, WV-5249, WV-5250, WV-5251, WV-5252, WV-5253, WV-5254, WV-5255, WV-5256, WV-5257, WV-5258, WV-5259, WV-5260, WV-5261, WV-5262, WV-5263, WV-5264, WV-5265, WV-5266, WV-5267, WV-5268, WV-5269, WV-5270, WV-5271, WV-5272, WV-5273, WV-5274, WV-5275, WV-5276, WV-5277, WV-5278, WV-5279, WV-5280, WV-5281, WV-5282, WV-5283, WV-5284, WV-5285, WV-5286, WV-10107, WV-10108, WV-10109, WV-10110, WV-10111, WV-10112, WV-10113, WV-10114, WV-10115, WV-10116, WV-10117, WV-10118, WV-10119, WV-10120, WV-10121, WV-10122, WV-10123, WV-10124, WV-10125, WV-10126, WV-10127, WV-10128, WV-10129, WV-10130, WV-10131, WV-10132, WV-10133, WV-10134, WV-10135, WV-10136, WV-10137, WV-10138, WV-10139, WV-10140, WV-10141, WV-10142, WV-10143, WV-10144, WV-10145, and WV-10146.

In some embodiments, the present disclosure pertains to a double-stranded RNAi agent, which comprises the strand of a single-stranded RNAi agent, which is annealed to a second strand. In some embodiments, the present disclosure pertains to a double-stranded HTT RNAi agent, which comprises the strand of a single-stranded HTT RNAi agent described herein, which is annealed to a second strand.

In some embodiments, oligonucleotides, such as double- or single-stranded HTT RNAi agents, comprise internucleotidic linkages and/or patterns thereof, nucleobase and patterns thereof, sugars and patterns thereof, backbone chiral center patterns, and/or additional chemical moieties described herein. In some embodiments, useful structural elements, such as nucleobases, sugars, internucleotidic linkages, linkage phosphorus stereochemistry, 5′-end groups (e.g., phosphate and analogs/derivatives thereof), additional chemical moieties, linkers, etc., and useful patterns and/or combinations thereof, are described in WO/2018/223056 and are incorporated herein by reference.

### Internucleotidic Linkages

In some embodiments, HTT oligonucleotides comprise base modifications, sugar modifications, and/or internucleotidic linkage modifications. Various internucleotidic linkages can be utilized in accordance with the present disclosure to link units comprising nucleobases, e.g., nucleosides. In some embodiments, provided oligonucleotides comprise both one or more modified internucleotidic linkages and one or more natural phosphate linkages. As widely known by those skilled in the art, natural phosphate linkages are widely found in natural DNA and RNA molecules; they have the structure of —OP(O)(OH)O—, connect sugars in the nucleosides in DNA and RNA, and may be in various salt forms, for example, at physiological pH (about 7.4), natural phosphate linkages are predominantly exist in salt forms with the anion being —OP(O)(O⁻)O—. A modified internucleotidic linkage, or a non-natural phosphate linkage, is an internucleotidic linkage that is not natural phosphate linkage or a salt form thereof. Modified internucleotidic linkages, depending on their structures, may also be in their salt forms. For example, as appreciated by those skilled in the art, phosphorothioate internucleotidic linkages which have the structure of —OP(O)(SH)O— may be in various salt forms, e.g., at physiological pH (about 7.4) with the anion being —OP(O)(S—)O—.

In some embodiments, a HTT oligonucleotide comprises an internucleotidic linkage which is a modified internucleotidic linkage, e.g., phosphorothioate, phosphorodithioate, methylphosphonate, phosphoroamidate, thiophosphate, 3′-thiophosphate, or 5′-thiophosphate.

In some embodiments, a modified internucleotidic linkage is a chiral internucleotidic linkage which comprises a chiral linkage phosphorus. In some embodiments, a chiral internucleotidic linkage is a phosphorothioate linkage. In some embodiments, a chiral internucleotidic linkage is a phosphorothioate linkage in the Rp or the Sp configuration (designated herein as * R or * S, respectively).

In some embodiments, a chiral internucleotidic linkage is a non-negatively charged internucleotidic linkage. In some embodiments, a chiral internucleotidic linkage is a neutral internucleotidic linkage. In some embodiments, a chiral internucleotidic linkage is chirally controlled with respect to its chiral linkage phosphorus. In some embodiments, a chiral internucleotidic linkage is stereochemically pure with respect to its chiral linkage phosphorus. In some embodiments, a chiral internucleotidic linkage is not chirally controlled. In some embodiments, a pattern of backbone chiral centers comprises or consists of positions and linkage phosphorus configurations of chirally controlled internucleotidic linkages (Rp or Sp) and positions of achiral internucleotidic linkages (e.g., natural phosphate linkages).

In some embodiments, an internucleotidic linkage comprises a P-modification, wherein a P-modification is a modification at a linkage phosphorus. In some embodiments, a modified internucleotidic linkage is a moiety which does not comprise a phosphorus but serves to link two sugars or two moieties that each independently comprises a nucleobase, e.g., as in peptide nucleic acid (PNA).

In some embodiments, an oligonucleotide comprises a modified internucleotidic linkage, e.g., those having the structure of Formula I, I-a, I-b, or I-c and described herein and/or in: WO 2018/022473, WO 2018/098264, WO 2018/223056, WO 2018/223073, WO 2018/223081, WO 2018/237194, WO 2019/032607, WO 2019/055951, and/or WO 2019/075357, the internucleotidic linkages (e.g., those of Formula I, I-a, I-b, I-c, etc.) of each of which are independently incorporated herein by reference.

In some embodiments, a modified internucleotidic linkage is a non-negatively charged internucleotidic linkage. In some embodiments, provided oligonucleotides comprise one or more non-negatively charged internucleotidic linkages. In some embodiments, a non-negatively charged internucleotidic linkage is a positively charged internucleotidic linkage. In some embodiments, a non-negatively charged internucleotidic linkage is a neutral internucleotidic linkage. In some embodiments, the present disclosure provides oligonucleotides comprising one or more neutral internucleotidic linkages. In some embodiments, a non-negatively charged internucleotidic linkage has the structure of Formula I-n-1, I-n-2, I-n-3, I-n-4, II, II-a-1, II-a-2, II-b-1, II-b-2, II-c-1, II-c-2, II-d-1, II-d-2, etc., or a salt form thereof, as described herein and/or in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, and/or WO 2019/075357, the non-negatively charged internucleotidic linkages (e.g., those of Formula I-n-1, I-n-2, I-n-3, I-n-4, II, II-a-1, II-a-2, II-b-1, II-b-2, II-c-1, II-c-2, II-d-1, II-d-2, etc., or a suitable salt form thereof) of each of which are independently incorporated herein by reference.

Non-limiting examples of oligonucleotides comprising a non-negatively charged internucleotidic linkage include: WV-19823, WV-19824, WV-19825, WV-19826, WV-19827, WV-19828, WV-19829, WV-19830, WV-19831, WV-19832, WV-19833, WV-19834, WV-19835, WV-19836, WV-19837, WV-19841, WV-19842, WV-19843, WV-19844, WV-19845, WV-19846, WV-19847, WV-19848, WV-19849, WV-19850, WV-19851, WV-19852, WV-19853, WV-19854, WV-16214, WV-16215, WV-16216, WV-19844, WV-19845, WV-19846, WV-19847, WV-19848, WV-19849, WV-19850, WV-19851, WV-19852, WV-19853, WV-19854, and WV-19855.

In some embodiments, a non-negatively charged internucleotidic linkage can improve the delivery and/or activity (e.g., ability to decrease the level, activity and/or expression of a HTT gene or a gene product thereof) of a HTT oligonucleotide.

In some embodiments, a modified internucleotidic linkage (e.g., a non-negatively charged internucleotidic linkage) comprises optionally substituted triazolyl. In some embodiments, a modified internucleotidic linkage (e.g., a non-negatively charged internucleotidic linkage) comprises optionally substituted alkynyl. In some embodiments, a modified internucleotidic linkage comprises a triazole or alkyne moiety. In some embodiments, a triazole moiety, e.g., a triazolyl group, is optionally substituted. In some embodiments, a triazole moiety, e.g., a triazolyl group) is substituted. In some embodiments, a triazole moiety is unsubstituted. In some embodiments, a modified internucleotidic linkage comprises an optionally substituted cyclic guanidine moiety. In some embodiments, a modified internucleotidic linkage comprises an optionally substituted cyclic guanidine moiety and has the structure of:

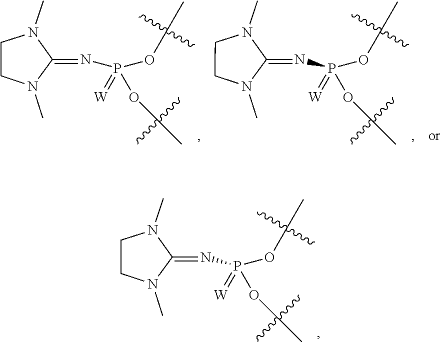

wherein W is O or S. In some embodiments, W is O. In some embodiments, W is S. In some embodiments, a non-negatively charged internucleotidic linkage is stereochemically controlled.

In some embodiments, a non-negatively charged internucleotidic linkage or a neutral internucleotidic linkage is an internucleotidic linkage comprising a triazole moiety. In some embodiments, a non-negatively charged internucleotidic linkage or a non-negatively charged internucleotidic linkage comprises an optionally substituted triazolyl group. In some embodiments, an internucleotidic linkage comprising a triazole moiety (e.g., an optionally substituted triazolyl group) has the structure of

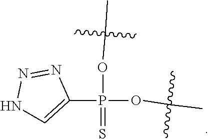 [SMILES: *OP(=S)(O*)c1c[nH]nn1]

In some embodiments, an internucleotidic linkage comprising a triazole moiety has the structure of

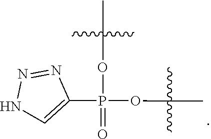 [SMILES: *OP(=O)(O*)c1c[nH]nn1]

In some embodiments, an internucleotidic linkage, e.g., a non-negatively charged internucleotidic linkage, a neutral internucleotidic linkage, comprises a cyclic guanidine moiety. In some embodiments, an internucleotidic linkage comprising a cyclic guanidine moiety has the structure of

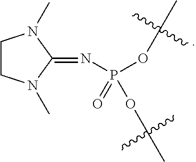 [SMILES: *OP(=O)(N=C1N(C)CCN1C)O*]

In some embodiments, a non-negatively charged internucleotidic linkage, or a neutral internucleotidic linkage, is or comprising a structure selected from

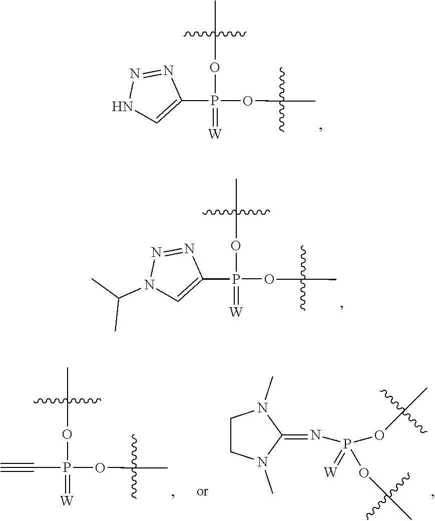

wherein W is O or S.

In some embodiments, an internucleotidic linkage comprises a Tmg group

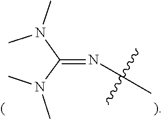 [SMILES: *N=C(N(C)C)N(C)C]

In some embodiments, an internucleotidic linkage comprises a Tmg group and has the structure of

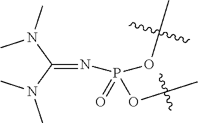 [SMILES: *OP(=O)(N=C(N(C)C)N(C)C)O*]

(the “Tmg internucleotidic linkage”). In some embodiments, neutral internucleotidic linkages include internucleotidic linkages of PNA and PMO, and an Tmg internucleotidic linkage.

In some embodiments, a non-negatively charged internucleotidic linkage comprises an optionally substituted 3-20 membered heterocyclyl or heteroaryl group having 1-10 heteroatoms. In some embodiments, a non-negatively charged internucleotidic linkage comprises an optionally substituted 3-20 membered heterocyclyl or heteroaryl group having 1-10 heteroatoms, wherein at least one heteroatom is nitrogen. In some embodiments, such a heterocyclyl or heteroaryl group is of a 5-membered ring. In some embodiments, such a heterocyclyl or heteroaryl group is of a 6-membered ring.

In some embodiments, a non-negatively charged internucleotidic linkage comprises an optionally substituted 5-20 membered heteroaryl group having 1-10 heteroatoms. In some embodiments, a non-negatively charged internucleotidic linkage comprises an optionally substituted 5-20 membered heteroaryl group having 1-10 heteroatoms, wherein at least one heteroatom is nitrogen. In some embodiments, a non-negatively charged internucleotidic linkage comprises an optionally substituted 5-6 membered heteroaryl group having 1-4 heteroatoms, wherein at least one heteroatom is nitrogen. In some embodiments, a non-negatively charged internucleotidic linkage comprises an optionally substituted 5-membered heteroaryl group having 1-4 heteroatoms, wherein at least one heteroatom is nitrogen. In some embodiments, a heteroaryl group is directly bonded to a linkage phosphorus. In some embodiments, a non-negatively charged internucleotidic linkage comprises an optionally substituted triazolyl group. In some embodiments, a non-negatively charged internucleotidic linkage comprises an unsubstituted triazolyl group,

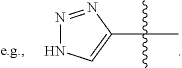 [SMILES: *c1c[nH]nn1]

In some embodiments, a non-negatively charged internucleotidic linkage comprises a substituted triazolyl group,

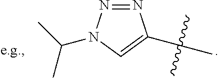 [SMILES: *c1cn(C(C)C)nn1]

In some embodiments, a non-negatively charged internucleotidic linkage comprises an optionally substituted 5-20 membered heterocyclyl group having 1-10 heteroatoms. In some embodiments, a non-negatively charged internucleotidic linkage comprises an optionally substituted 5-20 membered heterocyclyl group having 1-10 heteroatoms, wherein at least one heteroatom is nitrogen. In some embodiments, a non-negatively charged internucleotidic linkage comprises an optionally substituted 5-6 membered heterocyclyl group having 1-4 heteroatoms, wherein at least one heteroatom is nitrogen. In some embodiments, a non-negatively charged internucleotidic linkage comprises an optionally substituted 5-membered heterocyclyl group having 1-4 heteroatoms, wherein at least one heteroatom is nitrogen. In some embodiments, at least two heteroatoms are nitrogen. In some embodiments, a heterocyclyl group is directly bonded to a linkage phosphorus. In some embodiments, a heterocyclyl group is bonded to a linkage phosphorus through a linker, e.g., ═N— when the heterocyclyl group is part of a guanidine moiety who directed bonded to a linkage phosphorus through its ═N—. In some embodiments, a non-negatively charged internucleotidic linkage comprises an optionally substituted

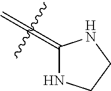 [SMILES: *=C1NCCN1]

group. In some embodiments, a non-negatively charged internucleotidic linkage comprises an substituted

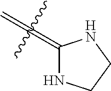 [SMILES: *=C1NCCN1]

group. In some embodiments, a non-negatively charged internucleotidic linkage comprises a

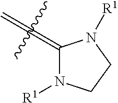

group. In some embodiments, each R¹ is independently optionally substituted C₁₋₆ alkyl. In some embodiments, each R¹ is independently methyl.

In some embodiments, a modified internucleotidic linkage, e.g., a non-negatively charged internucleotidic linkage, comprises a triazole or alkyne moiety, each of which is optionally substituted. In some embodiments, a modified internucleotidic linkage comprises a triazole moiety. In some embodiments, a modified internucleotidic linkage comprises a unsubstituted triazole moiety. In some embodiments, a modified internucleotidic linkage comprises a substituted triazole moiety. In some embodiments, a modified internucleotidic linkage comprises an alkyl moiety. In some embodiments, a modified internucleotidic linkage comprises an optionally substituted alkynyl group. In some embodiments, a modified internucleotidic linkage comprises an unsubstituted alkynyl group. In some embodiments, a modified internucleotidic linkage comprises a substituted alkynyl group. In some embodiments, an alkynyl group is directly bonded to a linkage phosphorus.

In some embodiments, a HTT oligonucleotide comprises different types of internucleotidic phosphorus linkages. In some embodiments, a chirally controlled oligonucleotide comprises at least one natural phosphate linkage and at least one modified (non-natural) internucleotidic linkage. In some embodiments, a HTT oligonucleotide comprises at least one natural phosphate linkage and at least one phosphorothioate. In some embodiments, a HTT oligonucleotide comprises at least one non-negatively charged internucleotidic linkage.

In some embodiments, a neutral or non-negatively charged internucleotidic linkage has the structure of any neutral or non-negatively charged internucleotidic linkage described in any of: U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, and/or WO 2019/075357,2607, WO2019/032612, WO 2019/055951, and/or WO 2019/075357, each neutral or non-negatively charged internucleotidic linkage of each of which is hereby incorporated by reference.

In some embodiments, a neutral internucleotidic linkage has the structure of formula II-d-2. In some embodiments, each R′ is independently optionally substituted C₁₋₆ aliphatic. In some embodiments, each R′ is independently optionally substituted C₁₋₆ alkyl. In some embodiments, each R′ is independently —CH₃. In some embodiments, each R^{s} is —H.

In some embodiments, a non-negatively charged internucleotidic linkage has the structure of

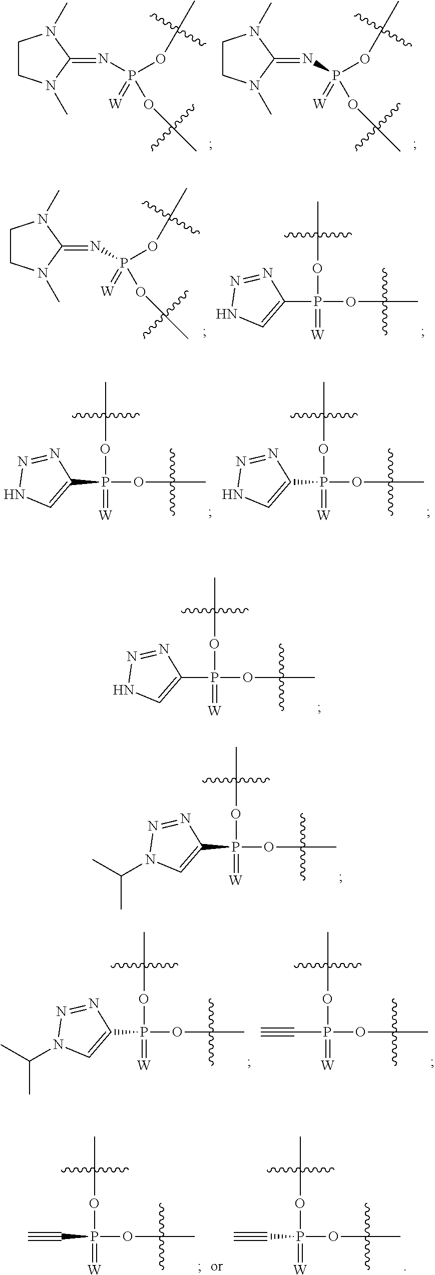

In some embodiments, W is O. In some embodiments, W is S. In some embodiments, a neutral internucleotidic linkage is a non-negatively charged internucleotidic linkage described above.

In some embodiments, provided oligonucleotides comprise 1 or more non-negatively charged internucleotidic linkage, and/or one or more internucleotidic linkages of Formula I, I-a, I-b, I-c, I-n-1, I-n-2, I-n-3, I-n-4, II, II-a-1, II-a-2, II-b-1, II-b-2, II-c-1, II-c-2, II-d-1, or II-d-2.

In some embodiments, a HTT oligonucleotide comprises a neutral internucleotidic linkage and a chirally controlled internucleotidic linkage. In some embodiments, a HTT oligonucleotide comprises a neutral internucleotidic linkage and a chirally controlled internucleotidic linkage which is not the neutral internucleotidic linkage. In some embodiments, a HTT oligonucleotide comprises a neutral internucleotidic linkage and a chirally controlled phosphorothioate internucleotidic linkage.

Without wishing to be bound by any particular theory, the present disclosure notes that a neutral internucleotidic linkage can be more hydrophobic than a phosphorothioate internucleotidic linkage (PS), which can be more hydrophobic than a natural phosphate linkage (PO). Typically, unlike a PS or PO, a neutral internucleotidic linkage bears less charge. Without wishing to be bound by any particular theory, the present disclosure notes that incorporation of one or more neutral internucleotidic linkages into a HTT oligonucleotide may increase oligonucleotides' ability to be taken up by a cell and/or to escape from endosomes. Without wishing to be bound by any particular theory, the present disclosure notes that incorporation of one or more neutral internucleotidic linkages can be utilized to modulate melting temperature of duplexes formed between a HTT oligonucleotide and its target nucleic acid.

Without wishing to be bound by any particular theory, the present disclosure notes that incorporation of one or more non-negatively charged internucleotidic linkages, e.g., neutral internucleotidic linkages, into a HTT oligonucleotide may be able to increase the oligonucleotide's ability to mediate a function such as gene knockdown. In some embodiments, a HTT oligonucleotide, e.g., a HTT oligonucleotide capable of mediating knockdown of level of a nucleic acid or a product encoded thereby comprises one or more non-negatively charged internucleotidic linkages. In some embodiments, a HTT oligonucleotide, e.g., a HTT oligonucleotide capable of mediating knockdown of expression of a HTT gene comprises one or more non-negatively charged internucleotidic linkages.

In some embodiments, a typical connection, as in natural DNA and RNA, is that an internucleotidic linkage forms bonds with two sugars (which can be either unmodified or modified as described herein). In many embodiments, as exemplified herein an internucleotidic linkage forms bonds through its oxygen atoms with one optionally modified ribose or deoxyribose at its 5′ carbon, and the other optionally modified ribose or deoxyribose at its 3′ carbon. In some embodiments, each nucleoside units connected by an internucleotidic linkage independently comprises a nucleobase which is independently an optionally substituted A, T, C, G, or U, or a substituted tautomer of A, T, C, G or U.

In some embodiments, a HTT oligonucleotide comprises an internucleotidic linkage wherein a negatively charged non-bridging oxygen of the canonical phosphodiester linkage is replaced by an uncharged alkyl substituent, such as a methyl (Met) or ethyl (Et) group, as in a P-alkyl phosphonate nucleic acid (phNA), such as a P-methyl or P-ethyl phNA. See, for example: Micklefield et al. 2001 Curr. Med. Chem. 8, 1157-1179; and Arangundy-Franklin et al. 2019 Nat. Chem. 11, 533-542.

In some embodiments, a HTT oligonucleotide is a phosphonomethyl-threosyl nucleic acid (tPhoNA) and/or comprises a phosphonomethyl-threosyl internucleotidic linkage. Liu et al. 2018 J. Am. Chem. Soc. 140, 6690-6699.

As appreciated by those skilled in the art, many other types of internucleotidic linkages may be utilized in accordance with the present disclosure, for example, those described in U.S. Pat. Nos. 3,687,808; 4,469,863; 4,476,301; 5,177,195; 5,023,243; 5,034,506; 5,166,315; 5,185,444; 5,188,897; 5,214,134; 5,216,141; 5,235,033; 5,264,423; 5,264,564; 5,276,019; 5,278,302; 5,286,717; 5,321,131; 5,399,676; 5,405,938; 5,405,939; 5,434,257; 5,453,496; 5,455,233; 5,466,677; 5,466,677; 5,470,967; 5,476,925; 5,489,677; 5,519,126; 5,536,821; 5,541,307; 5,541,316; 5,550,111; 5,561,225; 5,563,253; 5,571,799; 5,587,361; 5,596,086; 5,602,240; 5,608,046; 5,610,289; 5,618,704; 5,623,070; 5,625,050; 5,633,360; 5,64,562; 5,663,312; 5,677,437; 5,677,439; 6,160,109; 6,239,265; 6,028,188; 6,124,445; 6,169,170; 6,172,209; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; or RE39464. In some embodiments, a modified internucleotidic linkage is one described in U.S. Pat. No. 9,982,257, US 20170037399, US 20180216108, WO 2017192664, WO 2017015575, WO2017062862, WO 2018067973, WO 2017160741, WO 2017192679, WO 2017210647, WO 2018098264, PCT/US18/35687, PCT/US18/38835, or PCT/US18/51398, the nucleobases, sugars, internucleotidic linkages, chiral auxiliaries/reagents, and technologies for oligonucleotide synthesis (reagents, conditions, cycles, etc.) of each of which is independently incorporated herein by reference.

In some embodiments, each internucleotidic linkage in a HTT oligonucleotide is independently selected from a natural phosphate linkage, a phosphorothioate linkage, and a non-negatively charged internucleotidic linkage (e.g., n001). In some embodiments, each internucleotidic linkage in a HTT oligonucleotide is independently selected from a natural phosphate linkage, a phosphorothioate linkage, and a neutral internucleotidic linkage (e.g., n001).

In some embodiments, a HTT oligonucleotide comprises one or more nucleotides that independently comprise a phosphorus modification prone to “autorelease” under certain conditions. That is, under certain conditions, a particular phosphorus modification is designed such that it self-cleaves from the oligonucleotide to provide, e.g., a natural phosphate linkage. Certain examples of such phosphorus modification groups can be found in U.S. Pat. No. 9,982,257. In some embodiments, an autorelease group comprises a morpholino group. In some embodiments, an autorelease group is characterized by the ability to deliver an agent to the internucleotidic phosphorus linker, which agent facilitates further modification of the phosphorus atom such as, e.g., desulfurization. In some embodiments, the agent is water and the further modification is hydrolysis to form a natural phosphate linkage.

In some embodiments, a HTT oligonucleotide comprises one or more internucleotidic linkages that improve one or more pharmaceutical properties and/or activities of the oligonucleotide. It is well documented in the art that certain oligonucleotides are rapidly degraded by nucleases and exhibit poor cellular uptake through the cytoplasmic cell membrane (Poijarvi-Virta et al., Curr. Med. Chem. (2006), 13(28); 3441-65; Wagner et al., Med. Res. Rev. (2000), 20(6):417-51; Peyrottes et al., Mini Rev. Med. Chem. (2004), 4(4):395-408; Gosselin et al., (1996), 43(1):196-208; Bologna et al., (2002), Antisense & Nucleic Acid Drug Development 12:33-41). Vives et al., (Nucleic Acids Research (1999), 27(20):4071-76) reported that tert-butyl SATE pro-oligonucleotides displayed markedly increased cellular penetration compared to the parent oligonucleotide under certain conditions.

In some embodiments, the present disclosure demonstrates that, in at least some cases, Sp internucleotidic linkages, among other things, at the 5′- and/or 3′-end can improve oligonucleotide stability. In some embodiments, the present disclosure demonstrates that, among other things, natural phosphate linkages and/or Rp internucleotidic linkages can improve removal of oligonucleotides from a system. As appreciated by a person having ordinary skill in the art, various assays known in the art can be utilized to assess such properties in accordance with the present disclosure.

Various types of internucleotidic linkages may be utilized in combination of other structural elements, e.g., sugars, to achieve desired oligonucleotide properties and/or activities. For example, the present disclosure routinely utilizes modified internucleotidic linkages and modified sugars, optionally with natural phosphate linkages and natural sugars, in designing oligonucleotides. In some embodiments, the present disclosure provides a HTT oligonucleotide comprising one or more modified sugars.

In some embodiments, the present disclosure provides a HTT oligonucleotide comprising one or more modified sugars and one or more modified internucleotidic linkages, one or more of which may be chirally controlled.

In some embodiments, in a HTT oligonucleotide, chirally controlled internucleotidic linkages can appear in a particular pattern, which can affect one or more activity and/or property of the oligonucleotide.

### HTT Oligonucleotide Compositions and Stereochemistry

Among other things, the present disclosure provides various HTT oligonucleotide compositions. In some embodiments, the present disclosure provides oligonucleotide compositions of oligonucleotides described herein. In some embodiments, a HTT oligonucleotide composition, e.g., a HTT oligonucleotide composition, comprises a plurality of a HTT oligonucleotide described in the present disclosure. In some embodiments, a HTT oligonucleotide composition, e.g., a HTT oligonucleotide composition, is chirally controlled. In some embodiments, a HTT oligonucleotide composition, e.g., a HTT oligonucleotide composition, is not chirally controlled (stereorandom).

Linkage phosphorus of natural phosphate linkages is achiral. Linkage phosphorus of many modified internucleotidic linkages, e.g., phosphorothioate internucleotidic linkages, are chiral. In some embodiments, during preparation of oligonucleotide compositions (e.g., in traditional phosphoramidite oligonucleotide synthesis), configurations of chiral linkage phosphorus are not purposefully designed or controlled, creating non-chirally controlled (stereorandom) oligonucleotide compositions (substantially racemic preparations) which are complex, random mixtures of various stereoisomers (diastereoisomers)—for oligonucleotides with n chiral internucleotidic linkages (linkage phosphorus being chiral), typically 2ⁿ stereoisomers (e.g., when n is 10, 2¹⁰=1,032; when n is 20, 2²⁰=1,048,576). These stereoisomers have the same constitution, but differ with respect to the pattern of stereochemistry of their linkage phosphorus.

In some embodiments, stereorandom oligonucleotide compositions have sufficient properties and/or activities for certain purposes and/or applications. In some embodiments, stereorandom oligonucleotide compositions can be cheaper, easier and/or simpler to produce than chirally controlled oligonucleotide compositions.

However, in some embodiments, stereoisomers within stereorandom compositions may have different properties, activities, and/or toxicities, resulting in inconsistent therapeutic effects and/or unintended side effects by stereorandom compositions, particularly compared to certain chirally controlled oligonucleotide compositions of oligonucleotides of the same constitution.

In some embodiments, the present disclosure encompasses technologies for designing and preparing chirally controlled HTT oligonucleotide compositions. In some embodiments, the present disclosure provides chirally controlled oligonucleotide compositions, e.g., of many oligonucleotides in Table 1 which contain S and/or R in their stereochemistry/linkage. In some embodiments, a chirally controlled oligonucleotide composition comprises a controlled/pre-determined (not random as in stereorandom compositions) level of a plurality of oligonucleotides, wherein the oligonucleotides share the same linkage phosphorus stereochemistry at one or more chiral internucleotidic linkages (chirally controlled internucleotidic linkages). In some embodiments, the oligonucleotides share the same pattern of backbone chiral centers (stereochemistry of linkage phosphorus). In some embodiments, a pattern of backbone chiral centers is as described in the present disclosure. In some embodiments, the oligonucleotides are structural identical.

In some embodiments, level of a diastereopurity of a plurality of oligonucleotides in a composition can be determined as the product of the diastereopurity of each chirally controlled internucleotidic linkage in the oligonucleotides. In some embodiments, diastereopurity of an internucleotidic linkage connecting two nucleosides in a HTT oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions.

In some embodiments, all chiral internucleotidic linkages are chiral controlled, and the composition is a completely chirally controlled oligonucleotide composition. In some embodiments, not all chiral internucleotidic linkages are chiral controlled internucleotidic linkages, and the composition is a partially chirally controlled oligonucleotide composition.

Oligonucleotides may comprise or consist of various patterns of backbone chiral centers (patterns of stereochemistry of chiral linkage phosphorus). Certain useful patterns of backbone chiral centers are described in the present disclosure. In some embodiments, a plurality of oligonucleotides share a common pattern of backbone chiral centers, which is or comprises a pattern described in the present disclosure (e.g., as in “Linkage Phosphorus Stereochemistry and Patterns Thereof”, a pattern of backbone chiral centers of a chirally controlled oligonucleotide in Table 1, etc.).

In some embodiments, a chirally controlled oligonucleotide composition is chirally pure (or stereopure, stereochemically pure) oligonucleotide composition, wherein the oligonucleotide composition comprises a plurality of oligonucleotides, wherein the oligonucleotides are identical [including that each chiral element of the oligonucleotides, including each chiral linkage phosphorus, is independently defined (stereodefined)], and the composition does not contain other stereoisomers. A chirally pure (or stereopure, stereochemically pure) oligonucleotide composition of a HTT oligonucleotide stereoisomer does not contain other stereoisomers (as appreciated by those skilled in the art, one or more unintended stereoisomers may exist as impurities—example purities are descried in the present disclosure).

Chirally controlled oligonucleotide compositions can demonstrate a number of advantages over stereorandom oligonucleotide compositions. Among other things, chirally controlled oligonucleotide compositions are more uniform than corresponding stereorandom oligonucleotide compositions with respect to oligonucleotide structures. By controlling stereochemistry, compositions of individual stereoisomers can be prepared and assessed, so that chirally controlled oligonucleotide composition of stereoisomers with desired properties and/or activities can be developed. In some embodiments, chirally controlled oligonucleotide compositions provides better delivery, stability, clearance, activity, selectivity, and/or toxicity profiles compared to, e.g., corresponding stereorandom oligonucleotide compositions. In some embodiments, chirally controlled oligonucleotide compositions provide better efficacy, fewer side effects, and/or more convenient and effective dosage regimens. Among other things, patterns of backbone chiral centers as described herein can be utilized to provide controlled cleavage of oligonucleotide targets (e.g., transcripts such as pre-mRNA, mature mRNA, etc.; including control of cleavage sites, rate and/or extent of cleavage at cleavage sites, and/or overall rate and extent of cleavage, etc.) and greatly increased HTT target selectivity.

In some embodiments, a HTT oligonucleotide composition comprises one or more internucleotidic linkages which are stereocontrolled (chirally controlled; in some embodiments, stereopure) and one or more internucleotidic linkages which are stereorandom. In some embodiments, a HTT oligonucleotide composition comprises one or more internucleotidic linkages which are stereocontrolled (chirally controlled; in some embodiments, stereopure) and one or more internucleotidic linkages which are stereorandom.

In some embodiments, a HTT oligonucleotide composition comprises one or more internucleotidic linkages which are stereocontrolled (e.g., chirally controlled or stereopure) and one or more internucleotidic linkages which are stereorandom. Such oligonucleotides may target various targets and may have various base sequences, and may be capable of operating via one or more of various modalities (e.g., RNase H mechanism, steric hindrance, double- or single-stranded RNA interference, exon skipping modulation, CRISPR, aptamer, etc.).

Non-limiting examples of stereorandom oligonucleotide compositions, e.g., stereorandom HTT oligonucleotide compositions are described herein, including but not limited to: WV-1027, WV-1028, WV-1029, WV-1030, WV-1031, WV-1032, WV-1033, WV-1034, WV-1035, WV-1036, WV-1037, WV-1038, WV-1039, WV-1040, WV-1041, WV-1042, WV-1043, WV-1044, WV-1045, WV-1046, WV-1047, WV-1048, WV-1049, WV-1050, WV-1051, WV-1052, WV-1053, WV-1054, WV-1055, WV-1056, WV-1057, WV-1058, WV-1059, WV-1060, WV-1061, WV-1062, WV-1063, WV-1064, WV-1065, WV-1066, WV-1067, WV-1068, WV-1069, WV-1070, WV-1071, WV-1072, WV-2023, WV-2024, WV-2025, WV-2026, WV-2027, WV-2028, WV-2029, WV-2030, WV-2031, WV-2032, WV-2033, WV-2034, WV-2035, WV-2036, WV-2037, WV-2038, WV-2039, WV-2040, WV-2041, WV-2042, WV-2043, WV-2044, WV-2045, WV-2046, WV-2047, WV-2048, WV-2049, WV-2050, WV-2051, WV-2052, WV-2053, WV-2054, WV-2055, WV-2056, WV-2057, WV-2058, WV-2059, WV-2060, WV-2061, WV-2062, WV-2063, WV-2064, WV-2065, WV-2066, WV-2067, WV-2068, WV-2069, WV-2070, WV-2071, WV-2072, WV-2073, WV-2074, WV-2075, WV-2076, WV-2077, WV-2078, WV-2079, WV-2080, WV-2081, WV-2082, WV-2083, WV-2084, WV-2085, WV-2086, WV-2087, WV-2088, WV-2089, WV-2090, WV-2605, WV-2606, WV-2607, WV-2608, WV-2609, WV-2610, WV-2611, WV-2612, WV-2613, WV-2614, WV-2615, WV-2616, WV-2617, WV-2618, WV-2619, WV-2620, WV-13625, WV-13626, WV-13627, WV-13628, WV-13629, WV-13630, WV-13631, WV-13632, WV-13633, WV-13634, WV-13635, WV-13646, WV-13647, WV-13648, WV-13649, WV-13650, WV-13651, WV-13652, WV-13653, WV-13654, WV-13655, WV-13656, and WV-13667.

Non-limiting examples of stereopure (or chirally controlled) oligonucleotide compositions, e.g., stereopure (or chirally controlled) HTT oligonucleotide compositions, are described herein, including but not limited to: WV-2269, WV-2270, WV-2271, WV-2272, WV-2374, WV-2375, WV-2380, WV-2416, WV-2417, WV-2418, WV-2419, WV-2431, WV-2589, WV-2590, WV-2591, WV-2592, WV-2593, WV-2594, WV-2595, WV-2596, WV-2597, WV-2598, WV-2599, WV-2600, WV-2601, WV-2602, WV-2603, WV-2604, WV-2659, WV-2671, WV-2672, WV-2673, WV-2674, WV-2675, WV-2676, WV-2682, WV-2683, WV-2684, WV-2685, WV-2686, WV-2687, WV-2688, WV-2689, WV-2690, WV-2691, WV-2692, WV-2732, WV-13952, WV-13953, WV-13954, WV-13955, WV-13956, WV-13957, WV-13958, WV-13959, WV-13960, WV-13961, WV-13962, WV-14059, WV-14060, WV-14061, WV-14062, WV-14063, WV-14064, WV-14065, WV-14066, WV-14067, WV-14068, WV-14069, WV-14070, WV-14071, WV-14072, WV-14073, WV-14074, WV-14075, WV-14076, WV-14077, WV-14078, WV-14079, WV-14080, WV-14081, WV-14082, WV-14083, WV-14084, WV-14085, WV-14086, WV-14092, WV-14093, WV-14094, WV-14095, WV-14096, WV-14097, WV-14098, WV-14099, WV-14100, WV-14101, WV-14133, WV-14134, WV-14135, WV-14136, WV-14137, WV-14138, WV-14139, and WV-14140.

Non-limiting examples of oligonucleotide compositions, e.g., HTT oligonucleotide compositions, that comprise one or more internucleotidic linkages which are stereocontrolled (e.g., chirally controlled or stereopure) and one or more internucleotidic linkages which are stereorandom include but are not limited to: WV-13636, WV-13637, WV-13638, WV-13639, WV-13640, WV-13641, WV-13642, WV-13643, WV-13644, WV-13645, WV-13657, WV-13658, WV-13659, WV-13660, WV-13661, WV-13662, WV-13663, WV-13664, WV-13665, WV-13666.

In some embodiments, the present disclosure provides a chirally controlled oligonucleotide composition, e.g., chirally controlled HTT oligonucleotide composition. In some embodiments, provided chirally controlled oligonucleotide compositions comprise a plurality of HTT oligonucleotides of the same constitution, and have one or more internucleotidic linkages. In some embodiments, a plurality of oligonucleotides, e.g., in a chirally controlled oligonucleotide composition, is a plurality of a HTT oligonucleotide selected from Table 1, wherein the oligonucleotide comprises at least one Rp or Sp linkage phosphorus in a chirally controlled internucleotidic linkage. In some embodiments, a plurality of oligonucleotides, e.g., in a chirally controlled oligonucleotide composition, is a plurality of a HTT oligonucleotide selected from Table 1, wherein each phosphorothioate internucleotidic linkage in the oligonucleotide is independently chirally controlled (each phosphorothioate internucleotidic linkage is independently Rp or Sp). In some embodiments, a HTT oligonucleotide composition, e.g., a HTT oligonucleotide composition is a substantially pure preparation of a single oligonucleotide in that oligonucleotides in the composition that are not the single oligonucleotide are impurities from the preparation process of the single oligonucleotide, in some case, after certain purification procedures. In some embodiments, a single oligonucleotide is a HTT oligonucleotide of Table 1, wherein each chiral internucleotidic linkage of the oligonucleotide is chirally controlled (e.g., indicated as S or R but not X in “Stereochemistry/Linkage”).

In some embodiments, a chirally controlled oligonucleotide composition can have, relative to a corresponding stereorandom oligonucleotide composition, increased activity and/or stability, increased delivery, and/or decreased ability to elicit adverse effects such as complement, TLR9 activation, etc. In some embodiments, a stereorandom (non-chirally controlled) oligonucleotide composition differs from a chirally controlled oligonucleotide composition in that its corresponding plurality of oligonucleotides do not contain any chirally controlled internucleotidic linkages but the stereorandom oligonucleotide composition is otherwise identical to the chirally controlled oligonucleotide composition.

In some embodiments, the present disclosure pertains to a chirally controlled HTT oligonucleotide composition which is capable of decreasing the level, activity or expression of a HTT gene or a gene product thereof.

In some embodiments, the present disclosure provides a chirally controlled HTT oligonucleotide composition which is capable of decreasing the level, activity or expression of a HTT gene or a gene product thereof, and comprises a plurality of oligonucleotides which share a common base sequence that is, comprises, or comprises a span (e.g., at least 10 or 15 contiguous bases) of a base sequence disclosed herein (e.g., in Table 1, wherein each T may be independently replaced with U and vice versa). In some embodiments, the present disclosure provides a chirally controlled HTT oligonucleotide composition which is capable of decreasing the level, activity or expression of a HTT gene or a gene product thereof, and comprises a plurality of oligonucleotides which share a common base sequence that is or comprises a base sequence disclosed herein (e.g., in Table 1, wherein each T may be independently replaced with U and vice versa). In some embodiments, the present disclosure provides a chirally controlled HTT oligonucleotide composition which is capable of decreasing the level, activity or expression of a HTT gene or a gene product thereof, and comprises a plurality of oligonucleotides which share a common base sequence that is a base sequence disclosed herein (e.g., in Table 1, wherein each T may be independently replaced with U and vice versa).

In some embodiments, a provided chirally controlled oligonucleotide composition is a chirally controlled HTT oligonucleotide composition comprising a plurality of HTT oligonucleotides. In some embodiments, a chirally controlled oligonucleotide composition is a chirally pure (or “stereochemically pure”) oligonucleotide composition. In some embodiments, the present disclosure provides a chirally pure oligonucleotide composition of a HTT oligonucleotide in Table 1, wherein each chiral internucleotidic linkage of the oligonucleotide is independently chirally controlled (Rp or Sp, e.g., can be determined from R or S but not X in “Stereochemistry/Linkage”). As one of ordinary skill in the art will understand, chemical selectivity rarely, if ever, achieves completeness (absolute 100%). In some embodiments, a chirally pure oligonucleotide composition comprises a plurality of oligonucleotides, wherein oligonucleotides of the plurality are structurally identical and all have the same structure (the same stereoisomeric form; in the context of oligonucleotide, typically the same diastereomeric form as typically multiple chiral centers exist in a HTT oligonucleotide), and the chirally pure oligonucleotide composition does not contain any other stereoisomers (in the context of oligonucleotide, typically diastereomers as typically multiple chiral centers exist in a HTT oligonucleotide; to the extent, e.g., achievable by stereoselective preparation). As appreciated by those skilled in the art, stereorandom (or “racemic”, “non-chirally controlled”) oligonucleotide compositions are random mixtures of many stereoisomers (e.g., 2ⁿ diastereoisomers wherein n is the number of chiral linkage phosphorus for oligonucleotides in which other chiral centers (e.g., carbon chiral centers in sugars) are chirally controlled each independently existing in one configuration and only chiral linkage phosphorus centers are not chirally controlled).

Certain data showing properties and/or activities of chirally controlled oligonucleotide composition, e.g., chirally controlled HTT oligonucleotide compositions in decreasing the level, activity and/or expression of a HTT gene or a gene product thereof, are shown in, for example, the Examples section of this document.

In some embodiments, the present disclosure provides a HTT oligonucleotide composition comprising oligonucleotides that comprise at least one chiral linkage phosphorus. In some embodiments, the present disclosure provides a HTT oligonucleotide composition comprising HTT oligonucleotides that comprise at least one chiral linkage phosphorus. In some embodiments, the present disclosure provides a HTT oligonucleotide composition in which the HTT oligonucleotides comprise a chirally controlled phosphorothioate internucleotidic linkage, wherein the linkage phosphorus has a Rp configuration. In some embodiments, the present disclosure provides a HTT oligonucleotide composition in which the HTT oligonucleotides comprise a chirally controlled phosphorothioate internucleotidic linkage, wherein the linkage phosphorus has a Sp configuration.

In some embodiments, compared to reference oligonucleotide compositions, provided chirally controlled oligonucleotide compositions (e.g., chirally controlled HTT oligonucleotide compositions) are surprisingly effective. In some embodiments, desired biological effects (e.g., as measured by decreased levels of mRNA, proteins, etc. whose levels are targeted for reduction) can be enhanced by more than 5, 10, 15, 20, 25, 30, 40, 50, or 100 fold (e.g., as measured by remaining levels of mRNA, proteins, etc.). In some embodiments, a change is measured by decrease of an undesired mRNA level compared to a reference condition. In some embodiments, a change is measured by increase of a desired mRNA level compared to a reference condition. In some embodiments, a change is measured by decrease of an undesired mRNA level compared to a reference condition. In some embodiments, a reference condition is absence of treatment, e.g., by a chirally controlled oligonucleotide composition. In some embodiments, a reference condition is a corresponding stereorandom composition of oligonucleotides having the same constitution.

In some embodiments, the present disclosure provides a chirally controlled oligonucleotide composition, e.g., a chirally controlled HTT oligonucleotide composition, wherein the linkage phosphorus of at least one chirally controlled internucleotidic linkage is Sp. In some embodiments, the present disclosure provides a chirally controlled oligonucleotide composition, e.g., a chirally controlled HTT oligonucleotide composition, wherein the majority of linkage phosphorus of chirally controlled internucleotidic linkages are Sp. In some embodiments, about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more, of all chirally controlled internucleotidic linkages (or of all chiral internucleotidic linkages, or of all internucleotidic linkages) are Sp. In some embodiments, the present disclosure provides a chirally controlled oligonucleotide composition, e.g., a chirally controlled HTT oligonucleotide composition, wherein the majority of chiral internucleotidic linkages are chirally controlled and are Sp at their linkage phosphorus. In some embodiments, the present disclosure provides a chirally controlled oligonucleotide composition, e.g., a chirally controlled HTT oligonucleotide composition, wherein each chiral internucleotidic linkage is chirally controlled and each chiral linkage phosphorus is Sp. In some embodiments, the present disclosure provides a chirally controlled oligonucleotide composition, e.g., chirally controlled HTT oligonucleotide composition, wherein at least one chirally controlled internucleotidic linkage has a Rp linkage phosphorus. In some embodiments, the present disclosure provides a chirally controlled oligonucleotide composition, e.g., a chirally controlled HTT oligonucleotide composition, wherein at least one chirally controlled internucleotidic linkage comprises a Rp linkage phosphorus and at least one chirally controlled internucleotidic linkage comprises a Sp linkage phosphorus.

In some embodiments, the present disclosure provides a chirally controlled oligonucleotide composition, wherein at least two chirally controlled internucleotidic linkages have different linkage phosphorus stereochemistry and/or different P-modifications relative to one another, wherein a P-modification is a modification at a linkage phosphorus. In some embodiments, the present disclosure provides a chirally controlled oligonucleotide composition, wherein at least two chirally controlled internucleotidic linkages have different stereochemistry relative to one another, and the pattern of the backbone chiral centers of the oligonucleotides is characterized by a repeating pattern of alternating stereochemisty.

In certain embodiments, the present disclosure provides a chirally controlled oligonucleotide composition comprising a plurality of oligonucleotides, wherein with in each of the oligonucleotides at least two individual internucleotidic linkages have different P-modifications relative to one another. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a natural phosphate linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a phosphorothioate internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a natural phosphate linkage and a phosphorothioate internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a phosphorothioate triester internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a natural phosphate linkage and a phosphorothioate triester internucleotidic linkage. In certain embodiments, the present disclosure provides a chirally controlled oligonucleotide composition comprising a plurality of oligonucleotides, wherein with in each of the oligonucleotides at least two individual internucleotidic linkages have different P-modifications relative to one another, and each of the oligonucleotide comprises a phosphorothioate internucleotidic linkage and a phosphorothioate triester internucleotidic linkage.

In some embodiments, the present disclosure provides a chirally controlled oligonucleotide composition, e.g., a chirally controlled HTT oligonucleotide composition, comprising a plurality of oligonucleotides which share a common base sequence that is the base sequence of a HTT oligonucleotide disclosed herein, wherein at least one internucleotidic linkage is chirally controlled.

### Stereochemistry and Patterns of Backbone Chiral Centers

In contrast to natural phosphate linkages, linkage phosphorus of chiral modified internucleotidic linkages, e.g., phosphorothioate internucleotidic linkages, are chiral. Among other things, the present disclosure provides technologies (e.g., oligonucleotides, compositions, methods, etc.) comprising control of stereochemistry of chiral linkage phosphorus in chiral internucleotidic linkages. In some embodiments, as demonstrated herein, control of stereochemistry can provide improved properties and/or activities, including desired stability, reduced toxicity, improved reduction of HTT nucleic acids, etc. In some embodiments, the present disclosure provides useful patterns of backbone chiral centers for oligonucleotides and/or regions thereof, which pattern is a combination of stereochemistry of each chiral linkage phosphorus (Rp or Sp) of chiral linkage phosphorus, indication of each achiral linkage phosphorus (Op, if any), etc. from 5′ to 3′. In some embodiments, patterns of backbone chiral centers can control cleavage patterns of HTT nucleic acids when they are contacted with provided oligonucleotides or compositions thereof in a cleavage system (e.g., in vitro assay, cells, tissues, organs, organisms, subjects, etc.). In some embodiments, patterns of backbone chiral centers improve cleavage efficiency and/or selectivity of HTT nucleic acids when they are contacted with provided oligonucleotides or compositions thereof in a cleavage system.

In some embodiments, a HTT oligonucleotide (or a wing, core, block or any portion thereof) can comprise any pattern of chiral centers described in any of: WO2017015555; WO2017192664; WO0201200366; WO2011/034072; WO2014/010718; WO2015/108046; WO2015/108047; WO2015/108048; WO 2011/005761; WO 2011/108682; WO 2012/039448; WO 2018/067973; WO2005/028494; WO2005/092909; WO2010/064146; WO2012/073857; WO2013/012758; WO2014/010250; WO2014/012081; WO2015/107425; WO2017/015555; WO2017/015575; WO2017/062862; WO2017/160741; WO2017/192664; WO2017/192679; WO2017/210647; WO2018/022473; or WO2018/098264, the patterns of chiral centers of which are incorporated by reference.

In some embodiments, oligonucleotides in a chirally controlled oligonucleotide composition each comprise at least two internucleotidic linkages that have different stereochemistry and/or different P-modifications relative to one another. In some embodiments, at least two internucleotidic linkages have different stereochemistry relative to one another, and the oligonucleotides each comprise a pattern of backbone chiral centers comprising alternating linkage phosphorus stereochemistry.

In some embodiments, a phosphorothioate triester linkage comprises a chiral auxiliary, which, for example, is used to control the stereoselectivity of a reaction, e.g., a coupling reaction in a HTT oligonucleotide synthesis cycle. In some embodiments, a phosphorothioate triester linkage does not comprise a chiral auxiliary. In some embodiments, a phosphorothioate triester linkage is intentionally maintained until and/or during the administration of the oligonucleotide composition to a subject.

In some embodiments, oligonucleotides are linked to a solid support. In some embodiments, a solid support is a support for oligonucleotide synthesis. In some embodiments, a solid support comprises glass. In some embodiments, a solid support is CPG (controlled pore glass). In some embodiments, a solid support is polymer. In some embodiments, a solid support is polystyrene. In some embodiments, the solid support is Highly Crosslinked Polystyrene (HCP). In some embodiments, the solid support is hybrid support of Controlled Pore Glass (CPG) and Highly Cross-linked Polystyrene (HCP). In some embodiments, a solid support is a metal foam. In some embodiments, a solid support is a resin. In some embodiments, oligonucleotides are cleaved from a solid support.

In some embodiments, purity, particularly stereochemical purity, and particularly diastereomeric purity of many oligonucleotides and compositions thereof wherein all other chiral centers in the oligonucleotides but the chiral linkage phosphorus centers have been stereodefined (e.g., carbon chiral centers in the sugars, which are defined in, e.g., phosphoramidites for oligonucleotide synthesis), can be controlled by stereoselectivity (as appreciated by those skilled in this art, diastereoselectivity in many cases of oligonucleotide synthesis wherein the oligonucleotide comprise more than one chiral centers) at chiral linkage phosphorus in coupling steps when forming chiral internucleotidic linkages. In some embodiments, a coupling step has a stereoselectivity (diastereoselectivity when there are other chiral centers) of 60% at the linkage phosphorus. After such a coupling step, the new internucleotidic linkage formed may be referred to have a 60% stereochemical purity (for oligonucleotides, typically diastereomeric purity in view of the existence of other chiral centers). In some embodiments, each coupling step independently has a stereoselectivity of at least 60%, 70%, 80%, 85%, 90%, 91%, 92%, 93%, 94%, 95%, 96%, 97%, 98%, 99%, or 99.5%. In some embodiments, each coupling step independently has a stereoselectivity of virtually 100%.

In some embodiments, a coupling step has a stereoselectivity of virtually 100% in that each detectable product from the coupling step analyzed by an analytical method (e.g., NMR, HPLC, etc.) has the intended stereoselectivity. In some embodiments, a chirally controlled internucleotidic linkage is typically formed with a stereoselectivity of at least 90%, 91%, 92%, 93%, 94%, 95%, 96%, 97%, 98%, 99.5% or virtually 100% (in some embodiments, at least 90%; in some embodiments, at least 95%; in some embodiments, at least 96%; in some embodiments, at least 97%; in some embodiments, at least 98%; in some embodiments, at least 99%). In some embodiments, each chirally controlled internucleotidic linkage independently has a stereochemical purity (typically diastereomeric purity for oligonucleotides with multiple chiral centers) of at least 90%, 91%, 92%, 93%, 94%, 95%, 96%, 97%, 98%, 99.5% or virtually 100% (in some embodiments, at least 90%; in some embodiments, at least 95%; in some embodiments, at least 96%; in some embodiments, at least 97%; in some embodiments, at least 98%; in some embodiments, at least 99%) at its chiral linkage phosphorus.

In some embodiments, at least 1, 2, 3, 4, 5, 6, 7, 8, 9, or 10 couplings of a monomer (as appreciated by those skilled in the art in many embodiments a phosphoramidite for oligonucleotide synthesis) independently have a stereoselectivity less than about 60%, 70%, 80%, 85%, or 90% [for oligonucleotide synthesis, typically diastereoselectivity with respect to formed linkage phosphorus chiral center(s)].

In some embodiments, a stereochemical purity, e.g., diastereomeric purity, is about 60%-100%.

In some embodiments, compounds of the present disclosure (e.g., oligonucleotides, chiral auxiliaries, etc.) comprise multiple chiral elements (e.g., multiple carbon and/or phosphorus (e.g., linkage phosphorus of chiral internucleotidic linkages) chiral centers). In some embodiments, at least 1, 2, 3, 4, 5, 6, 7, 8, 9 or more chiral elements of a provided compound (e.g., a HTT oligonucleotide) each independently have a diastereomeric purity as described herein.

As understood by a person having ordinary skill in the art, in some embodiments, diastereoselectivity of a coupling or diastereomeric purity of a chiral linkage phosphorus center can be assessed through the diastereoselectivity of a dimer formation or diastereomeric purity of a dimer prepared under the same or comparable conditions, wherein the dimer has the same 5′- and 3′-nucleosides and internucleotidic linkage.

Various technologies can be utilized for identifying or confirming stereochemistry of chiral elements (e.g., configuration of chiral linkage phosphorus) and/or patterns of backbone chiral centers, and/or for assessing stereoselectivity (e.g., diastereoselectivity of couple steps in oligonucleotide synthesis) and/or stereochemical purity (e.g., diastereomeric purity of internucleotidic linkages, compounds (e.g., oligonucleotides), etc.). Example technologies include NMR [e.g., 1D (one-dimensional) and/or 2D (two-dimensional)¹H-³¹P HETCOR (heteronuclear correlation spectroscopy)], HPLC, RP-HPLC, mass spectrometry, LC-MS, and cleavage of internucleotidic linkages by stereospecific nucleases, etc., which may be utilized individually or in combination. Example useful nucleases include benzonase, micrococcal nuclease, and svPDE (snake venom phosphodiesterase), which are specific for certain internucleotidic linkages with Rp linkage phosphorus (e.g., a Rp phosphorothioate linkage); and nuclease P1, mung bean nuclease, and nuclease Si, which are specific for internucleotidic linkages with Sp linkage phosphorus (e.g., a Sp phosphorothioate linkage). Without wishing to be bound by any particular theory, the present disclosure notes that, in at least some cases, cleavage of oligonucleotides by a particular nuclease may be impacted by structural elements, e.g., chemical modifications (e.g., 2′-modifications of a sugars), base sequences, or stereochemical contexts. For example, it is observed that in some cases, benzonase and micrococcal nuclease, which are specific for internucleotidic linkages with Rp linkage phosphorus, were unable to cleave an isolated Rp phosphorothioate internucleotidic linkage flanked by Sp phosphorothioate internucleotidic linkages.

In some embodiments, a plurality of HTT oligonucleotides share the same constitution. In some embodiments, a plurality of HTT oligonucleotides are identical (the same stereoisomer). In some embodiments, a chirally controlled oligonucleotide composition, e.g., a chirally controlled HTT oligonucleotide composition, is a stereopure oligonucleotide composition wherein oligonucleotides of the plurality are identical (the same stereoisomer), and the composition does not contain any other stereoisomers. Those skilled in the art will appreciate that one or more other stereoisomers may exist as impurities as processes, selectivities, purifications, etc. may not achieve completeness.

In some embodiments, a provided composition is characterized in that when it is contacted with a HTT nucleic acid [e.g., a HTT transcript (e.g., pre-mRNA, mature mRNA, other types of RNA, etc. that hybridizes with oligonucleotides of the composition)], levels of the HTT nucleic acid and/or a product encoded thereby (e.g., a protein) is reduced compared to that observed under a reference condition. In some embodiments, a reference condition is selected from the group consisting of absence of the composition, presence of a reference composition, and combinations thereof. In some embodiments, a reference condition is absence of the composition. In some embodiments, a reference condition is presence of a reference composition. In some embodiments, a reference composition is a composition whose oligonucleotides do not hybridize with the HTT nucleic acid. In some embodiments, a reference composition is a composition whose oligonucleotides do not comprise a sequence that is sufficiently complementary to the HTT nucleic acid. In some embodiments, a provided composition is a chirally controlled oligonucleotide composition and a reference composition is a non-chirally controlled oligonucleotide composition which is otherwise identical but is not chirally controlled (e.g., a racemic preparation of oligonucleotides of the same constitution as oligonucleotides (e.g., of a plurality, of a particular oligonucleotide type, etc.) in the chirally controlled oligonucleotide composition).

As noted above and understood in the art, in some embodiments, the base sequence of a HTT oligonucleotide may refer to the identity and/or modification status of nucleoside residues (e.g., of sugar and/or base components, relative to standard naturally occurring nucleotides such as adenine, cytosine, guanosine, thymine, and uracil) in the oligonucleotide and/or to the hybridization character (i.e., the ability to hybridize with particular complementary residues) of such residues.

As demonstrated herein, oligonucleotide structural elements (e.g., patterns of sugar modifications, backbone linkages, backbone chiral centers, backbone phosphorus modifications, etc.) and combinations thereof can provide surprisingly improved properties and/or bioactivities.

In some embodiments, oligonucleotide compositions are capable of reducing the expression, level and/or activity of a HTT gene or a gene product thereof. In some embodiments, oligonucleotide compositions are capable of reducing in the expression, level and/or activity of a HTT gene or a gene product thereof by sterically blocking translation after annealing to a HTT mRNA (e.g., pre-mRNA or mature mRNA), by cleaving the mRNA. In some embodiments, provided HTT oligonucleotide compositions are capable of reducing the expression, level and/or activity of a HTT gene or a gene product thereof. In some embodiments, provided HTT oligonucleotide compositions are capable of reducing in the expression, level and/or activity of a HTT gene or a gene product thereof by sterically blocking translation after annealing to a HTT mRNA, by cleaving HTT mRNA (pre-mRNA or mature mRNA), and/or by altering or interfering with mRNA splicing.

In some embodiments, a HTT oligonucleotide composition, e.g., a HTT oligonucleotide composition, is a substantially pure preparation of a single oligonucleotide stereoisomer, e.g., a HTT oligonucleotide stereoisomer, in that oligonucleotides in the composition that are not of the oligonucleotide stereoisomer are impurities from the preparation process of said oligonucleotide stereoisomer, in some case, after certain purification procedures.

In some embodiments, the present disclosure provides oligonucleotides and oligonucleotide compositions that are chirally controlled, and in some embodiments, stereopure. For instance, in some embodiments, a provided composition contains non-random or controlled levels of one or more individual oligonucleotide types. In some embodiments, oligonucleotides of the same oligonucleotide type are identical.

### Sugars

Various sugars, including modified sugars, can be utilized in accordance with the present disclosure. In some embodiments, the present disclosure provides sugar modifications and patterns thereof optionally in combination with other structural elements (e.g., internucleotidic linkage modifications and patterns thereof, pattern of backbone chiral centers thereof, etc.) that when incorporated into oligonucleotides can provide improved properties and/or activities.

The most common naturally occurring nucleosides comprise ribose sugars (e.g., in RNA) or deoxyribose sugars (e.g., in DNA) linked to the nucleobases adenosine (A), cytosine (C), guanine (G), thymine (T) or uracil (U). In some embodiments, a sugar, e.g., various sugars in many oligonucleotides in Table 1 (unless otherwise notes), is a natural DNA sugar (in DNA nucleic acids or oligonucleotides, having the structure of

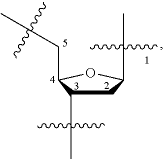

wherein a nucleobase is attached to the 1′ position, and the 3′ and 5′ positions are connected to internucleotidic linkages (as appreciated by those skilled in the art, if at the 5′-end of a HTT oligonucleotide, the 5′ position may be connected to a 5′-end group (e.g., —OH), and if at the 3′-end of a HTT oligonucleotide, the 3′ position may be connected to a 3′-end group (e.g., —OH). In some embodiments, a sugar is a natural RNA sugar (in RNA nucleic acids or oligonucleotides, having the structure of

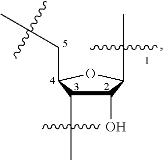

wherein a nucleobase is attached to the 1′ position, and the 3′ and 5′ positions are connected to internucleotidic linkages (as appreciated by those skilled in the art, if at the 5′-end of a HTT oligonucleotide, the 5′ position may be connected to a 5′-end group (e.g., —OH), and if at the 3′-end of a HTT oligonucleotide, the 3′ position may be connected to a 3′-end group (e.g., —OH). In some embodiments, a sugar is a modified sugar in that it is not a natural DNA sugar or a natural RNA sugar. Among other things, modified sugars may provide improved stability. In some embodiments, modified sugars can be utilized to alter and/or optimize one or more hybridization characteristics. In some embodiments, modified sugars can be utilized to alter and/or optimize HTT nucleic acid recognition. In some embodiments, modified sugars can be utilized to optimize Tm. In some embodiments, modified sugars can be utilized to improve oligonucleotide activities.

Sugars can be bonded to internucleotidic linkages at various positions. As non-limiting examples, internucleotidic linkages can be bonded to the 2′, 3′, 4′ or 5′ positions of sugars. In some embodiments, as most commonly in natural nucleic acids, an internucleotidic linkage connects with one sugar at the 5′ position, and another sugar at the 3′ position.

In some embodiments, a sugar is an optionally substituted natural DNA or RNA sugar. In some embodiments, a substituent, a sugar, modified sugar and/or sugar modification is one described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, and/or WO 2019/075357, the substituents, sugar modifications, and modified sugars of each of which are independently incorporated herein by reference). Various such sugars are utilized in Table 1.

In some embodiments, a sugar is a bicyclic sugar. In some embodiments, a sugar is selected from LNA sugars, BNA sugars, cEt sugars, etc.

In some embodiments, a sugar is a 2′-OMe, 2′-MOE, 2′-F, LNA (locked nucleic acid), ENA (ethylene bridged nucleic acid), BNA(NMe) (Methylamino bridged nucleic acid), 2′-F ANA (2′-F arabinose), alpha-DNA (alpha-D-ribose), 2′/5′ ODN (e.g., 2′/5′ linked oligonucleotide), Inv (inverted sugar, e.g., inverted desoxyribose), AmR (Amino-Ribose), ThioR (Thio-ribose), HNA (hexose nucleic acid), CeNA (cyclohexene nucleic acid), or MOR (Morpholino) sugar.

In some embodiments, provided oligonucleotides comprise one or more modified sugars. In some embodiments, provided oligonucleotides comprise one or more modified sugars and one or more natural sugars.

Examples of bicyclic sugars include alpha-L-methyleneoxy (4′-CH₂—O-2′) LNA, beta-D-methyleneoxy (4′-CH₂—O-2′) LNA, ethyleneoxy (4′-(CH₂)₂—O-2′) LNA, aminooxy (4′-CH₂—O—N(R)-2′) LNA, and oxyamino (4′-CH₂—N(R)—O-2′) LNA. In some embodiments, a bicyclic sugar, e.g., a LNA or BNA sugar, is sugar having at least one bridge between two sugar carbons. In some embodiments, a bicyclic sugar in a nucleoside may have the stereochemical configurations of alpha-L-ribofuranose or beta-D-ribofuranose. In some embodiments, a sugar is a sugar described in WO 1999014226. In some embodiments, a 4′-2′ bicyclic sugar or 4′ to 2′ bicyclic sugar is a bicyclic sugar comprising a furanose ring which comprises a bridge connecting the 2′ carbon atom and the 4′ carbon atom of the sugar ring. In some embodiments, a bicyclic sugar, e.g., a LNA or BNA sugar, comprises at least one bridge between two pentofuranosyl sugar carbons. In some embodiments, a LNA or BNA sugar, comprises at least one bridge between the 4′ and the 2′ wo pentofuranosyl sugar carbons.

In some embodiments, a bicyclic sugar may be further defined by isomeric configuration.

Certain modified sugars (e.g., bicyclic sugars that have 4′ to 2′ bridging groups such as 4′-CH₂—O-2′ and 4′-CH₂—S—2′), their preparation and/or uses are described in Kumar et al., Bioorg. Med. Chem. Lett., 1998, 8, 2219-2222; WO 1999014226; etc. 2′-amino-BNAs, which may provide conformationally restriction and high-affinity in some cases are described in, e.g., Singh et al., J. Org. Chem., 1998, 63, 10035-10039. In addition, 2′-amino- and 2′-methylamino-BNA sugars and the thermal stability of their duplexes with complementary RNA and DNA strands have been previously reported.

In some embodiments, sugars are bicyclic sugars having a hydrocarbon bridge, e.g., a 4′-(CH₂)₃-2′ bridge, 4′-CH═CH—CH₂-2′ bridge, etc. (e.g., Freier et al., Nucleic Acids Research, 1997, 25(22), 4429-4443; Albaek et al., J. Org. Chem., 2006, 71, 7731-7740; etc.). Example preparation of such bicyclic sugars and nucleosides along with their oligomerization and biochemical studies were reported, e.g., Srivastava et al., J. Am. Chem. Soc. 2007, 129(26), 8362-8379.

In some embodiments, a bicyclic sugar is a sugar of alpha-L-methyleneoxy (4′-CH₂—O-2′) BNA, beta-D-methyleneoxy (4′-CH₂—O-2′) BNA, ethyleneoxy (4′-(CH₂)₂—O-2′) BNA, aminooxy (4′-CH₂—O—N(R)-2′) BNA, oxyamino (4′-CH₂—N(R)—O-2′) BNA, methyl(methyleneoxy) (4′-CH(CH₃)—O-2′) BNA (also referred to as constrained ethyl or cEt), methylene-thio (4′-CH₂—S—2′) BNA, methylene-amino (4′-CH₂—N(R)-2′) BNA, methyl carbocyclic (4′-CH₂—CH(CH₃)-2′) BNA, propylene carbocyclic (4′-(CH₂)₃-2′) BNA, or vinyl BNA.

In some embodiments, a sugar modification is a modification described in U.S. Pat. No. 9,006,198. In some embodiments, a modified sugar is described in U.S. Pat. No. 9,006,198. In some embodiments, a sugar modification is a modification described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, and/or WO 2019/075357, the sugar modifications and modified sugars of each of which are independently incorporated herein by reference.

In some embodiments a modified sugar is one described in U.S. Pat. Nos. 5,658,873, 5,118,800, 5,393,878, 5,514,785, 5,627,053, 7,034,133; 7,084,125, 7,399,845, 5,319,080, 5,591,722, 5,597,909, 5,466,786, 6,268,490, 6,525,191, 5,519,134, 5,576,427, 6,794,499, 6,998,484, 7,053,207, 4,981,957, 5,359,044, 6,770,748, 7,427,672, 5,446,137, 6,670,461, 7,569,686, 7,741,457, 8,022,193, 8,030,467, 8,278,425, 5,610,300, 5,646,265, 8,278,426, 5,567,811, 5,700,920, 8,278,283, 5,639,873, 5,670,633, 8,314,227, US 2008/0039618 or US 2009/0012281.

In some embodiments, a sugar modification is 2′-OMe, 2′-MOE, 2′-LNA, 2′-F, 5′-vinyl, or S-cEt. In some embodiments, a modified sugar is a sugar of FRNA, FANA, or morpholino. In some embodiments, a HTT oligonucleotide comprises a nucleic acid analog, e.g., GNA, LNA, PNA, TNA, FHNA (F-THP or 3′-fluoro tetrahydropyran), MNA (mannitol nucleic acid, e.g., Leumann 2002 Bioorg. Med. Chem. 10: 841-854), ANA (anitol nucleic acid), or morpholino, or a portion thereof. In some embodiments, a sugar modification replaces a natural sugar with another cyclic or acyclic moiety. Examples of such moieties are widely known in the art, e.g., those used in morpholino, glycol nucleic acids, etc. and may be utilized in accordance with the present disclosure. As appreciated by those skilled in the art, when utilized with modified sugars, in some embodiments internucleotidic linkages may be modified, e.g., as in morpholino, PNA, etc.

In some embodiments, a sugar is a 6′-modified bicyclic sugar that have either (R) or (S)-chirality at the 6-position, e.g., those described in U.S. Pat. No. 7,399,845. In some embodiments, a sugar is a 5′-modified bicyclic sugar that has either (R) or (S)-chirality at the 5-position, e.g., those described in US 20070287831.

In some embodiments, a modified sugar contains one or more substituents at the 2′ position (typically one substituent, and often at the axial position) independently selected from —F; —CF₃, —CN, —N₃, —NO, —NO₂, —OR^{•}, —SR^{•}, or —N(R′)₂, wherein each R′ is independently described in the present disclosure; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In some embodiments, a substituent is —O(CH₂), —OCH₃, —O(CH₂)ₙNH₂, MOE, DMAOE, or DMAEOE, wherein n is from 1 to about 10. In some embodiments, a modified sugar is one described in WO 2001/088198; and Martin et al., Helv. Chim. Acta, 1995, 78, 486-504. In some embodiments, a modified sugar comprises one or more groups selected from a substituted silyl group, an RNA cleaving group, a reporter group, a fluorescent label, an intercalator, a group for improving the pharmacokinetic properties of a nucleic acid, a group for improving the pharmacodynamic properties of a nucleic acid, or other substituents having similar properties. In some embodiments, modifications are made at one or more of the 2′, 3′, 4′, or 5′ positions, including the 3′ position of the sugar on the 3′-terminal nucleoside or in the 5′ position of the 5′-terminal nucleoside.

In some embodiments, the 2′-OH of a ribose is replaced with a group selected from —H, —F; —CF₃, —CN, —N₃, —NO, —NO₂, —OR′, —SR′, or —N(R′)₂, wherein each R′ is independently described in the present disclosure; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In some embodiments, the 2′-OH is replaced with —H (deoxyribose). In some embodiments, the 2′-OH is replaced with —F. In some embodiments, the 2′-OH is replaced with —OR′. In some embodiments, the 2′-OH is replaced with —OMe. In some embodiments, the 2′-OH is replaced with —OCH₂CH₂OMe.

In some embodiments, a sugar modification is a 2′-modification. Commonly used 2′-modifications include but are not limited to 2‘—OR’, wherein R¹ is not hydrogen and is as described in the present disclosure. In some embodiments, a modification is 2′-OR, wherein R is optionally substituted C₁₋₆ aliphatic. In some embodiments, a modification is 2′-OR, wherein R is optionally substituted C₁₋₆ alkyl. In some embodiments, a modification is 2′-OMe. In some embodiments, a modification is 2′-MOE. In some embodiments, a 2′-modification is S-cEt. In some embodiments, a modified sugar is an LNA sugar. In some embodiments, a 2′-modification is —F. In some embodiments, a 2′-modification is FANA. In some embodiments, a 2′-modification is FRNA. In some embodiments, a sugar modification is a 5′-modification, e.g., 5′-Me. In some embodiments, a sugar modification changes the size of the sugar ring. In some embodiments, a sugar modification is the sugar moiety in FHNA.

In some embodiments, a sugar modification replaces a sugar moiety with another cyclic or acyclic moiety. Examples of such moieties are widely known in the art, including but not limited to those used in morpholino (optionally with its phosphorodiamidate linkage), glycol nucleic acids, etc.

In some embodiments, one or more of the sugars of a HTT oligonucleotide are modified. In some embodiments, a modified sugar comprises a 2′-modification. In some embodiments, each modified sugar independently comprises a 2′-modification. In some embodiments, a 2′-modification is 2′-OR. In some embodiments, a 2′-modification is a 2′-OMe. In some embodiments, a 2′-modification is a 2′-MOE. In some embodiments, a 2′-modification is an LNA sugar modification. In some embodiments, a 2′-modification is 2′-F. In some embodiments, each sugar modification is independently a 2′-modification. In some embodiments, each sugar modification is independently 2′-OR or 2′-F. In some embodiments, each sugar modification is independently 2′-OR or 2′-F, wherein R¹ is optionally substituted C₁₋₆ alkyl. In some embodiments, each sugar modification is independently 2′-OR or 2′-F, wherein at least one is 2′-F. In some embodiments, each sugar modification is independently 2′-OR or 2′-F, wherein R¹ is optionally substituted C₁₋₆ alkyl, and wherein at least one is 2′-OR. In some embodiments, each sugar modification is independently 2′-OR or 2′-F, wherein at least one is 2′-F, and at least one is 2′-OR. In some embodiments, each sugar modification is independently 2′-OR or 2′-F, wherein R¹ is optionally substituted C₁₋₆ alkyl, and wherein at least one is 2′-F, and at least one is 2′-OR. In some embodiments, each sugar modification is independently 2′-OR. In some embodiments, each sugar modification is independently 2′-OR, wherein R¹ is optionally substituted C₁₋₆ alkyl. In some embodiments, each sugar modification is 2′-OMe. In some embodiments, each sugar modification is 2′-MOE. In some embodiments, each sugar modification is independently 2′-OMe or 2′-MOE. In some embodiments, each sugar modification is independently 2′-OMe, 2′-MOE, or a LNA sugar.

In some embodiments, a modified sugar is an optionally substituted ENA sugar. In some embodiments, a sugar is one described in, e.g., Seth et al., J Am Chem Soc. 2010 Oct. 27; 132(42): 14942-14950. In some embodiments, a modified sugar is a sugar in XNA (xenonucleic acid), for instance, arabinose, anhydrohexitol, threose, 2′fluoroarabinose, or cyclohexene.

Modified sugars include cyclobutyl or cyclopentyl moieties in place of a pentofuranosyl sugar. Representative examples of such modified sugars include those described in U.S. Pat. Nos. 4,981,957, 5,118,800, 5,319,080, or U.S. Pat. No. 5,359,044. In some embodiments, the oxygen atom within the ribose ring is replaced by nitrogen, sulfur, selenium, or carbon. In some embodiments, —O— is replaced with —N(R′)—, —S—, —Se— or —C(R′)₂—. In some embodiments, a modified sugar is a modified ribose wherein the oxygen atom within the ribose ring is replaced with nitrogen, and wherein the nitrogen is optionally substituted with an alkyl group (e.g., methyl, ethyl, isopropyl, etc.).

A non-limiting example of modified sugars is glycerol, which is part of glycerol nucleic acids (GNAs), e.g., as described in Zhang, R et al., J. Am. Chem. Soc., 2008, 130, 5846-5847; Zhang L, et al., J. Am. Chem. Soc., 2005, 127, 4174-4175 and Tsai C H et al., PNAS, 2007, 14598-14603.

A flexible nucleic acid (FNA) is based on a mixed acetal aminal of formyl glycerol, e.g., as described in Joyce G F et al., PNAS, 1987, 84, 4398-4402 and Heuberger B D and Switzer C, J. Am. Chem. Soc., 2008, 130, 412-413.

In some embodiments, a HTT oligonucleotide, and/or a modified nucleoside thereof, comprises a sugar or modified sugar described in: WO 2018/022473, WO 2018/098264, WO 2018/223056, WO 2018/223073, WO 2018/223081, WO 2018/237194, WO 2019/032607, WO 2019/055951, and/or WO 2019/075357, the sugars and modified sugars of each of which are independently incorporated herein by reference.

In some embodiments, one or more hydroxyl group in a sugar is optionally and independently replaced with halogen, R′—N(R′)₂, —OR′, or —SR′, wherein each R′ is independently described in the present disclosure.

In some embodiments, a modified nucleoside is any modified nucleoside described in: WO 2018/022473, WO 2018/098264, WO 2018/223056, WO 2018/223073, WO 2018/223081, WO 2018/237194, WO 2019/032607, WO 2019/055951, and/or WO 2019/075357, the modified nucleosides of each of which are independently incorporated herein by reference.

In some embodiments, a modified nucleoside comprises a modified sugar and has the structure of

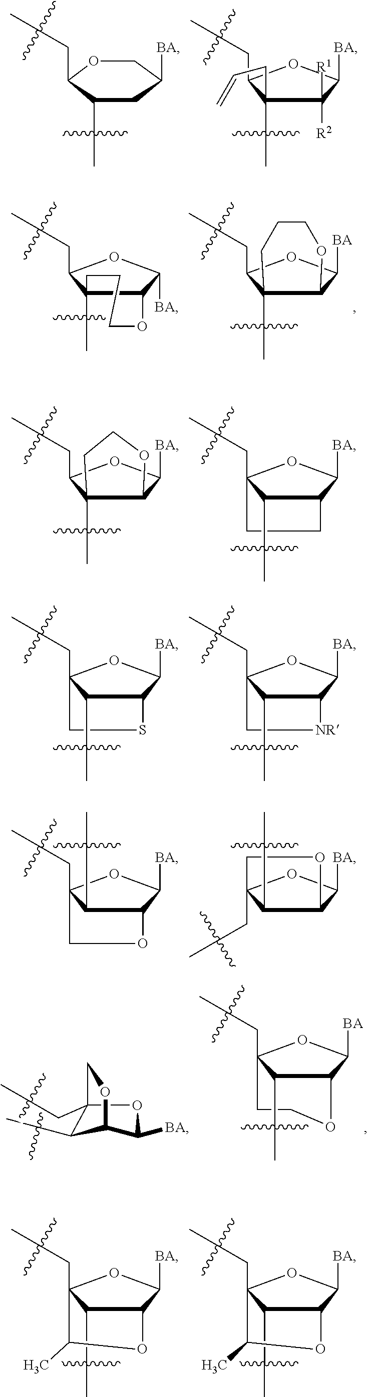

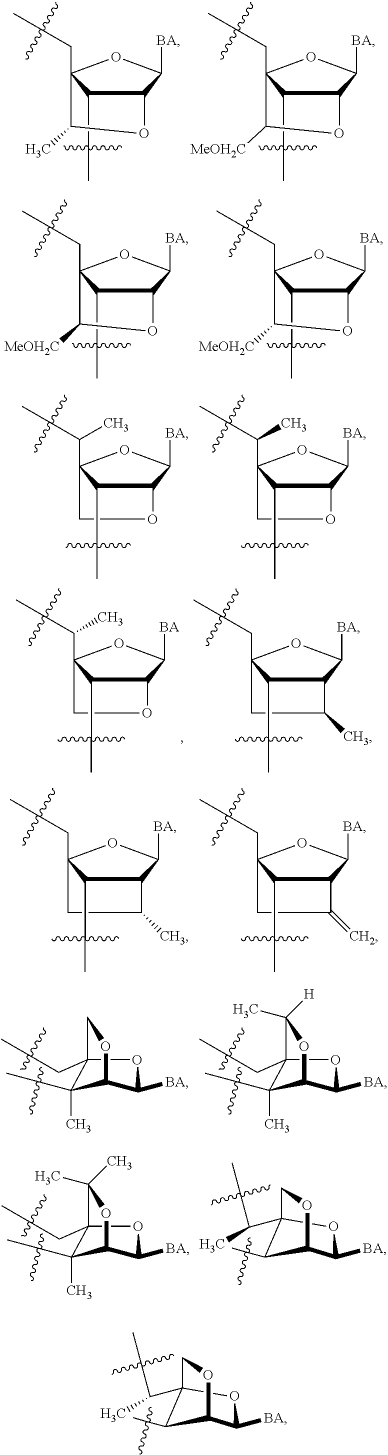

wherein each of R¹ and R² is independently —H, —F, —OMe, -MOE, or optionally substituted C₁₋₆ alkyl, R′ is as described in the present disclosure, and BA is a nucleobase as described in the present disclosure. In some embodiments, a sugar is a sugar of such nucleoside. In some embodiments, a sugar is a sugar of 2′-thio-LNA, HNA, beta-D-oxy-LNA, beta-D-thio-LNA, beta-D-amino-LNA, xylo-LNA, alpha-L-LNA, ENA, beta-D-ENA, methylphosphonate-LNA, (R,S)-cEt, (R)-cEt, (S)-cEt, (R,S)-cMOE, (R)-cMOE, (S)-cMOE, (R,S)-5′-Me-LNA, (R)-5′-Me-LNA, (S)-5′-Me-LNA, (S)-Me cLNA, methylene-cLNA, 3′-methyl-alpha-L-LNA, (R)-6′-methyl-alpha-L-LNA, (S)-5′-methyl-alpha-L-LNA, or (R)-5′-Me-alpha-L-LNA. Example modified sugars are additionally described in WO 2008/101157, WO 2007/134181, WO 2016/167780 or US 20050130923.

Modified sugars, their preparation methods, uses, etc., that can be utilized in accordance with the present disclosure include those described in any of: A. Eschenmoser, Science (1999), 284:2118; M. Bohringer et al., Helv. Chim. Acta (1992), 75:1416-1477; M. Egli et al., J. Am. Chem. Soc. (2006), 128(33):10847-56; A. Eschenmoser in Chemical Synthesis: Gnosis to Prognosis, C. Chatgilialoglu and V. Sniekus, Ed., (Kluwer Academic, Netherlands, 1996), p. 293; K.-U. Schoning et al., Science (2000), 290:1347-1351; A. Eschenmoser et al., Helv. Chim. Acta (1992), 75:218; J. Hunziker et al., Helv. Chim. Acta (1993), 76:259; G. Otting et al., Helv. Chim. Acta (1993), 76:2701; K. Groebke et al., Helv. Chim. Acta (1998), 81:375; or A. Eschenmoser, Science (1999), 284:2118. Modified sugars and methods thereof can also be found in Verma, S. et al. Annu. Rev. Biochem. 1998, 67, 99-134 and references therein. 2′-fluoro modified sugars and methods are described in, e.g., Kawasaki et. al., J. Med. Chem., 1993, 36, 831-841); 2′-MOE modified sugars and methods are described in, e.g., Martin, P. Helv. Chim. Acta 1996, 79, 1930-1938; and LNA sugars and methods are described in, e.g., Wengel, J. Acc. Chem. Res. 1999, 32, 301-310. In some embodiments, modified sugars and methods thereof are those described in WO 2012/030683. Useful modified sugars and methods thereof are also described in Gryaznov, S; Chen, J.-K. J. Am. Chem. Soc. 1994, 116, 3143; Hendrix et al. 1997 Chem. Eur. J. 3: 110; Hyrup et al. 1996 Bioorg. Med. Chem. 4: 5; Jepsen et al. 2004 Oligo. 14: 130-146; Jones et al. J. Org. Chem. 1993, 58, 2983; Koizumi et al. 2003 Nuc. Acids Res. 12: 3267-3273; Koshkin et al. 1998 Tetrahedron 54: 3607-3630; Kumar et al. 1998 Bioo. Med. Chem. Let. 8: 2219-2222; Lauritsen et al. 2002 Chem. Comm. 5: 530-531; Lauritsen et al. 2003 Bioo. Med. Chem. Lett. 13: 253-256; Mesmaeker et al. Angew. Chem., Int. Ed. Engl. 1994, 33, 226; Morita et al. 2001 Nucl. Acids Res. Supp. 1: 241-242; Morita et al. 2002 Bioo. Med. Chem. Lett. 12: 73-76; Morita et al. 2003 Bioo. Med. Chem. Lett. 2211-2226; Nielsen et al. 1997 Chem. Soc. Rev. 73; Nielsen et al. 1997 J. Chem. Soc. Perkins Transl. 1: 3423-3433; Obika et al. 1997 Tetrahedron Lett. 38 (50): 8735-8; Obika et al. 1998 Tetrahedron Lett. 39: 5401-5404; Pallan et al. 2012 Chem. Comm. 48: 8195-8197; Petersen et al. 2003 TRENDS Biotech. 21: 74-81; Rajwanshi et al. 1999 Chem. Commun. 1395-1396; Schultz et al. 1996 Nucleic Acids Res. 24: 2966; Seth et al. 2009 J. Med. Chem. 52: 10-13; Seth et al. 2010 J. Med. Chem. 53: 8309-8318; Seth et al. 2010 J. Org. Chem. 75: 1569-1581; Seth et al. 2012 Bioo. Med. Chem. Lett. 22: 296-299; Seth et al. 2012 Mol. Ther-Nuc. Acids. 1, e47; Seth, Punit P; Siwkowski, Andrew; Allerson, Charles R; Vasquez, Guillermo; Lee, Sam; Prakash, Thazha P; Kinberger, Garth; Migawa, Michael T; Gaus, Hans; Bhat, Balkrishen; et al. From Nucleic Acids Symposium Series (2008), 52(1), 553-554; Singh et al. 1998 Chem. Comm. 1247-1248; Singh et al. 1998 J. Org. Chem. 63: 10035-39; Singh et al. 1998 J. Org. Chem. 63: 6078-6079; Sorensen 2003 Chem. Comm. 2130-2131; Ts'o et al. Ann. N. Y. Acad. Sci. 1988, 507, 220; Van Aerschot et al. 1995 Angew. Chem. Int. Ed. Engl. 34: 1338; and Vasseur et al. J. Am. Chem. Soc. 1992, 114, 4006. Certain bicyclic sugars, their preparation and uses that can be utilized in accordance with the present disclosure include WO 2007090071 and WO 2016/079181.

In some embodiments, a modified sugar is an optionally substituted pentose or hexose. In some embodiments, a modified sugar is an optionally substituted pentose. In some embodiments, a modified sugar is an optionally substituted hexose. In some embodiments, a modified sugar is an optionally substituted ribose or hexitol. In some embodiments, a modified sugar is an optionally substituted ribose. In some embodiments, a modified sugar is an optionally substituted hexitol.

In some embodiments, a sugar modification is 5′-vinyl (R or S), 5′-methyl (R or S), 2′-SH, 2′-F, 2′-OCH₃, 2′-OCH₂CH₃, 2′-OCH₂CH₂F or 2′-O(CH₂)₂₀CH₃. In some embodiments, a substituent at the 2′ position, e.g., a 2′-modification, is allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ), wherein each allyl, amino and alkyl is optionally substituted, and each of R₁, Rₘ and Rₙ is independently R′ as described in the present disclosure. In some embodiments, each of R₁, Rₘ and Rₙ is independently —H or optionally substituted C₁-C₁₀ alkyl.

Certain bicyclic sugars are described in, e.g., Chattopadhyaya et al., J. Org. Chem., 2009, 74, 118-134, WO 2008154401, WO 2009006478, Srivastava et al., J. Am. Chem. Soc., 2007, 129(26) 8362-8379; Frieden et al., Nucleic Acids Research, 2003, 21, 6365-6372; Elayadi et al., Curr. Opinion Inverts. Drugs, 2001, 2, 558-561; Braasch et al., Chem. Biol., 2001, 8, 1-7; Oram et al., Curr. Opinion Mol Ther., 2001, 3,239-243; Wahlestedt et al., Proc. Natl Acad. Sci. U.S.A, 2000, 97, 5633-5638; Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; U.S. Pat. Nos. 7,399,845; 7,053,207; 7,034,133; 6,794,499; 6,770,748; 6,670,461; 6,525,191; 6,268,490; 7,741,457; 8,501,805; 8,546,556; US 20080039618; US 20070287831; US 20040171570; WO 2007134181; WO 2005021570; WO 2004106356; WO 2009006478; WO 2008154401; WO 2008150729; etc.

In some embodiments, a sugar is a tetrahydropyran or THP sugar. In some embodiments, a modified nucleoside is tetrahydropyran nucleoside or THP nucleoside which is a nucleoside having a six-membered tetrahydropyran sugar substituted for a pentofuranosyl residue in typical natural nucleosides. THP sugars and/or nucleosides include those used in hexitol nucleic acid (HNA), anitol nucleic acid (ANA), mannitol nucleic acid (MNA) (e.g., Leumann, Bioorg. Med. Chem., 2002, 10, 841-854) or fluoro HNA (FHNA).

In some embodiments, sugars comprise rings having more than 5 atoms and/or more than one heteroatom, e.g., morpholino sugars which are described in e.g., Braasch et al., Biochemistry, 2002, 41, 4503-4510; U.S. Pat. Nos. 5,698,685; 5,166,315; 5,185,444; 5,034,506; etc.).

As those skilled in the art will appreciate, modifications of sugars, nucleobases, internucleotidic linkages, etc. can and are often utilized in combination in oligonucleotides, e.g., see various oligonucleotides in Table 1. For example, a combination of sugar modification and nucleobase modification is 2′-F (sugar) 5-methyl (nucleobase) modified nucleosides. See WO 2008101157 for additional examples. In some embodiments, a combination is replacement of a ribosyl ring oxygen atom with S and substitution at the 2′-position (e.g., as described in US 20050130923), or 5′-substitution of a bicyclic sugar (e.g., see WO 2007134181, wherein a 4′-CH₂—O-2′ bicyclic nucleoside is further substituted at the 5′ position with a 5′-methyl or a 5′-vinyl group).

In some embodiments, provided oligonucleotides comprise one or more modified cyclohexenyl nucleosides, which is a nucleoside having a six-membered cyclohexenyl in place of the pentofuranosyl residue in naturally occurring nucleosides. Example cyclohexenyl nucleosides and preparation and uses thereof are described in, e.g., WO 2010036696; Robeyns et al., J. Am. Chem. Soc., 2008, 130(6), 1979-1984; Horvath et al., Tetrahedron Letters, 2007, 48, 3621-3623; Nauwelaerts et al., J. Am. Chem. Soc., 2007, 129(30), 9340-9348; Gu et al., Nucleosides, Nucleotides & Nucleic Acids, 2005, 24(5-7), 993-998; Nauwelaerts et al., Nucleic Acids Research, 2005, 33(8), 2452-2463; Robeyns et al., Acta Crystallographica, Section F: Structural Biology and Crystallization Communications, 2005, F61(6), 585-586; Gu et al., Tetrahedron, 2004, 60(9), 2111-2123; Gu et al., Oligonucleotides, 2003, 13(6), 479-489; Wang et al., J. Org. Chem., 2003, 68, 4499-4505; Verbeure et al., Nucleic Acids Research, 2001, 29(24), 4941-4947; Wang et al., J. Org. Chem., 2001, 66, 8478-82; Wang et al., Nucleosides, Nucleotides & Nucleic Acids, 2001, 20(4-7), 785-788; Wang et al., J. Am. Chem., 2000, 122, 8595-8602; WO 2006047842; WO 2001049687; etc.

Many monocyclic, bicyclic and tricyclic ring systems are suitable as sugar surrogates (modified sugars) and may be utilized in accordance with the present disclosure. See, e.g., Leumann, Christian J. Bioorg. & Med. Chem., 2002, 10, 841-854. Such ring systems can undergo various additional substitutions to further enhance their properties and/or activities.

In some embodiments, a 2′-modified sugar is a furanosyl sugar modified at the 2′ position. In some embodiments, a 2′-modification is halogen, —R′ (wherein R′ is not —H), —OR′ (wherein R′ is not —H), —SR^{•}, —N(R′)₂, optionally substituted —CH₂—CH═CH₂, optionally substituted alkenyl, or optionally substituted alkynyl. In some embodiments, a 2′-modifications is selected from —O[(CH₂)ₙO]ₘCH₃, —O(CH₂)ₙNH₂, —O(CH₂)ₙCH₃, —O(CH₂)ₙF, —O(CH₂)ₙONH₂, —OCH₂C(═O)N(H)CH₃, and —O(CH₂)ₙON[(CH₂)ₙCH₃]₂, wherein each n and m is independently from 1 to about 10. In some embodiments, a 2′-modification is optionally substituted C₁-C₁₂ alkyl, optionally substituted alkenyl, optionally substituted alkynyl, optionally substituted alkaryl, optionally substituted aralkyl, optionally substituted —O-alkaryl, optionally substituted —O-aralkyl, —SH, —SCH₃, —OCN, —Cl, —Br, —CN, —F, —CF₃, —OCF₃, —SOCH₃, —SO₂CH₃, —ONO₂, —NO₂, —N₃, —NH₂, optionally substituted heterocycloalkyl, optionally substituted heterocycloalkaryl, optionally substituted aminoalkylamino, optionally substituted polyalkylamino, substituted silyl, a reporter group, an intercalator, a group for improving pharmacokinetic properties, a group for improving the pharmacodynamic properties, and other substituents. In some embodiments, a 2′-modification is a 2′-MOE modification (e.g., see Baker et al., J. Biol. Chem., 1997, 272, 11944-12000). In some cases, a 2′-MOE modification has been reported as having improved binding affinity compared to unmodified sugars and to some other modified nucleosides, such as 2′-O-methyl, 2′-O-propyl, and 2′-O-aminopropyl. Oligonucleotides having the 2′-MOE modification have also been reported to be capable of inhibiting gene expression with promising features for in vivo use (see, e.g., Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926; etc.).

In some embodiments, a 2′-modified or 2′-substituted sugar or nucleoside is a sugar or nucleoside comprising a substituent at the 2′ position of the sugar which is other than —H (typically not considered a substituent) or —OH. In some embodiments, a 2′-modified sugar is a bicyclic sugar comprising a bridge connecting two carbon atoms of the sugar ring one of which is the 2′ carbon. In some embodiments, a 2′-modification is non-bridging, e.g., allyl, amino, azido, thio, optionally substituted —O-allyl, optionally substituted —O—C₁-C₁₀ alkyl, —OCF₃, —O(CH₂)₂OCH₃, 2′-O(CH₂)₂SCH₃, —O(CH₂)₂ON(Rₘ)(Rₙ), or —OCH₂C(═O)N(Rₘ)(Rₙ), where each Rₘ and Rₙ is independently —H or optionally substituted C₁-C₁₀ alkyl.

Certain modified sugars, their preparation and uses are described in U.S. Pat. Nos. 4,981,957, 5,118,800, 5,319,080, 5,359,044, 5,393,878, 5,446,137, 5,466,786, 5,514,785, 5,519,134, 5,567,811, 5,576,427, 5,591,722, 5,597,909, 5,610,300, 5,627,053, 5,639,873, 5,646,265, 5,670,633, 5,700,920, 5,792,847, 6,600,032 and WO 2005121371.

In some embodiments, a sugar is the sugar of N-methanocarba, LNA, cMOE BNA, cEt BNA, α-L-LNA or related analogs, HNA, Me-ANA, MOE-ANA, Ara-FHNA, FHNA, R-6′-Me-FHNA, S-6′-Me-FHNA, ENA, or c-ANA. In some embodiments, a modified internucleotidic linkage is C3-amide (e.g., sugar that has the amide modification attached to the C3′, Mutisya et al. 2014 Nucleic Acids Res. 2014 Jun. 1; 42(10): 6542-6551), formacetal, thioformacetal, MMI [e.g., methylene(methylimino), Peoc'h et al. 2006 Nucleosides and Nucleotides 16 (7-9)], a PMO (phosphorodiamidate linked morpholino) linkage (which connects two sugars), or a PNA (peptide nucleic acid) linkage. In some embodiments, examples of internucleotidic linkages and/or sugars are described in Allerson et al. 2005 J. Med. Chem. 48: 901-4; BMCL 2011 21: 1122; BMCL 2011 21: 588; BMCL 2012 22: 296; Chattopadhyaya et al. 2007 J. Am. Chem. Soc. 129: 8362; Chem. Bio. Chem. 2013 14: 58; Curr. Prot. Nucl. Acids Chem. 2011 1.24.1; Egli et al. 2011 J. Am. Chem. Soc. 133: 16642; Hendrix et al. 1997 Chem. Eur. J. 3: 110; Hyrup et al. 1996 Bioorg. Med. Chem. 4: 5; Imanishi 1997 Tet. Lett. 38: 8735; J. Am. Chem. Soc. 1994, 116, 3143; J. Med. Chem. 2009 52: 10; J. Org. Chem. 2010 75: 1589; Jepsen et al. 2004 Oligo. 14: 130-146; Jones et al. J. Org. Chem. 1993, 58, 2983; Jung et al. 2014 ACIEE 53: 9893; Kodama et al. 2014 AGDS; Koizumi 2003 BMC 11: 2211; Koizumi et al. 2003 Nuc. Acids Res. 12: 3267-3273; Koshkin et al. 1998 Tetrahedron 54: 3607-3630; Kumar et al. 1998 Bioo. Med. Chem. Let. 8: 2219-2222; Lauritsen et al. 2002 Chem. Comm. 5: 530-531; Lauritsen et al. 2003 Bioo. Med. Chem. Lett. 13: 253-256; Lima et al. 2012 Cell 150: 883-894; Mesmaeker et al. Angew. Chem., Int. Ed. Engl. 1994, 33, 226; Migawa et al. 2013 Org. Lett. 15: 4316; Mol. Ther. Nucl. Acids 2012 1: e47; Morita et al. 2001 Nucl. Acids Res. Supp. 1: 241-242; Morita et al. 2002 Bioo. Med. Chem. Lett. 12: 73-76; Morita et al. 2003 Bioo. Med. Chem. Lett. 2211-2226; Murray et al. 2012 Nucl. Acids Res. 40: 6135; Nielsen et al. 1997 Chem. Soc. Rev. 73; Nielsen et al. 1997 J. Chem. Soc. Perkins Transl. 1: 3423-3433; Obika et al. 1997 Tetrahedron Lett. 38 (50): 8735-8; Obika et al. 1998 Tetrahedron Lett. 39: 5401-5404; Obika et al. 2008 J. Am. Chem. Soc. 130: 4886; Obika et al. 2011 Org. Lett. 13: 6050; Oestergaard et al. 2014 JOC 79: 8877; Pallan et al. 2012 Biochem. 51: 7; Pallan et al. 2012 Chem. Comm. 48: 8195-8197; Petersen et al. 2003 TRENDS Biotech. 21: 74-81; Prakash et al. 2010 J. Med. Chem. 53: 1636; Prakash et al. 2015 Nucl. Acids Res. 43: 2993-3011; Prakash et al. 2016 Bioorg. Med. Chem. Lett. 26: 2817-2820; Rajwanshi et al. 1999 Chem. Commun. 1395-1396; Schultz et al. 1996 Nucleic Acids Res. 24: 2966; Seth et al. 2008 Nucl. Acid Sym. Ser. 52: 553; Seth et al. 2009 J. Med. Chem. 52: 10-13; Seth et al. 2010 J. Am. Chem. Soc. 132: 14942; Seth et al. 2010 J. Med. Chem. 53: 8309-8318; Seth et al. 2010 J. Org. Chem. 75: 1569-1581; Seth et al. 2011 BMCL 21: 4690; Seth et al. 2012 Bioo. Med. Chem. Lett. 22: 296-299; Seth et al. 2012 Mol. Ther-Nuc. Acids. 1, e47; Seth et al., Nucleic Acids Symposium Series (2008), 52(1), 553-554; Singh et al. 1998 Chem. Comm. 1247-1248; Singh et al. 1998 J. Org. Chem. 63: 10035-39; Singh et al. 1998 J. Org. Chem. 63: 6078-6079; Sorensen 2003 Chem. Comm. 2130-2131; Starrup et al. 2010 Nucl. Acids Res. 38: 7100; Swayze et al. 2007 Nucl. Acids Res. 35: 687; Ts'o et al. Ann. N. Y. Acad. Sci. 1988, 507, 220; Van Aerschot et al. 1995 Angew. Chem. Int. Ed. Engl. 34: 1338; Vasseur et al. J. Am. Chem. Soc. 1992, 114, 4006; WO 2007090071; WO 2016079181; U.S. Pat. Nos. 6,326,199; 6,066,500; or U.S. Pat. No. 6,440,739.

Various additional sugars useful for preparing oligonucleotides or analogs thereof are known in the art and may be utilized in accordance with the present disclosure.

### Nucleobases

Various nucleobases may be utilized in provided oligonucleotides in accordance with the present disclosure. In some embodiments, a nucleobase is a natural nucleobase, the most commonly occurring ones being A, T, C, G and U. In some embodiments, a nucleobase is a modified nucleobase in that it is not A, T, C, G or U. In some embodiments, a nucleobase is optionally substituted A, T, C, G or U, or a substituted tautomer of A T, C, G or U. In some embodiments, a nucleobase is optionally substituted A, T, C, G or U, e.g., 5mC, 5-hydroxymethyl C, etc. In some embodiments, a nucleobase is alkyl-substituted A, T, C, G or U. In some embodiments, a nucleobase is A. In some embodiments, a nucleobase is T. In some embodiments, a nucleobase is C. In some embodiments, a nucleobase is G. In some embodiments, a nucleobase is U. In some embodiments, a nucleobase is 5mC. In some embodiments, a nucleobase is substituted A, T, C, G or U. In some embodiments, a nucleobase is a substituted tautomer of A, T, C, G or U. In some embodiments, substitution protects certain functional groups in nucleobases to minimize undesired reactions during oligonucleotide synthesis. Suitable technologies for nucleobase protection in oligonucleotide synthesis are widely known in the art and may be utilized in accordance with the present disclosure. In some embodiments, modified nucleobases improves properties and/or activities of oligonucleotides. For example, in many cases, 5mC may be utilized in place of C to modulate certain undesired biological effects, e.g., immune responses. In some embodiments, when determining sequence identity, a substituted nucleobase having the same hydrogen-bonding pattern is treated as the same as the unsubstituted nucleobase, e.g., 5mC may be treated the same as C [e.g., a HTT oligonucleotide having 5mC in place of C (e.g., AT5mCG) is considered to have the same base sequence as a HTT oligonucleotide having C at the corresponding location(s) (e.g., ATCG)].

In some embodiments, a HTT oligonucleotide comprises one or more A, T, C, G or U. In some embodiments, a HTT oligonucleotide comprises one or more optionally substituted A, T, C, G or U. In some embodiments, a HTT oligonucleotide comprises one or more 5-methylcytidine, 5-hydroxymethylcytidine, 5-formylcytosine, or 5-carboxylcytosine. In some embodiments, a HTT oligonucleotide comprises one or more 5-methylcytidine. In some embodiments, each nucleobase in a HTT oligonucleotide is selected from the group consisting of optionally substituted A, T, C, G and U, and optionally substituted tautomers of A, T, C, G and U. In some embodiments, each nucleobase in a HTT oligonucleotide is optionally protected A, T, C, G and U. In some embodiments, each nucleobase in a HTT oligonucleotide is optionally substituted A, T, C, G or U. In some embodiments, each nucleobase in a HTT oligonucleotide is selected from the group consisting of A, T, C, G, U, and 5mC.

In some embodiments, a nucleobase is optionally substituted 2AP or DAP. In some embodiments, a nucleobase is optionally substituted 2AP. In some embodiments, a nucleobase is optionally substituted DAP. In some embodiments, a nucleobase is 2AP. In some embodiments, a nucleobase is DAP.

As appreciated by those skilled in the art, various nucleobases are known in the art and can be utilized in accordance with the present disclosure, e.g., those described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, and/or WO 2019/075357, the sugar, base, and internucleotidic linkage modifications of each of which are independently incorporated herein by reference. In some embodiments, nucleobases are protected and useful for oligonucleotide synthesis.

In some embodiments, a nucleobase is a natural nucleobase or a modified nucleobase derived from a natural nucleobase. Examples include uracil, thymine, adenine, cytosine, and guanine optionally having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Certain examples of modified nucleobases are disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313. In some embodiments, a modified nucleobase is substituted uracil, thymine, adenine, cytosine, or guanine. In some embodiments, a modified nucleobase is a functional replacement, e.g., in terms of hydrogen bonding and/or base pairing, of uracil, thymine, adenine, cytosine, or guanine. In some embodiments, a nucleobase is optionally substituted uracil, thymine, adenine, cytosine, 5-methylcytosine, or guanine. In some embodiments, a nucleobase is uracil, thymine, adenine, cytosine, 5-methylcytosine, or guanine.

In some embodiments, a provided HTT oligonucleotide comprises one or more 5-methylcytosine. In some embodiments, the present disclosure provides a HTT oligonucleotide whose base sequence is disclosed herein, e.g., in Table 1, wherein each T may be independently replaced with U and vice versa, and each cytosine is optionally and independently replaced with 5-methylcytosine or vice versa. As appreciated by those skilled in the art, in some embodiments, 5mC may be treated as C with respect to base sequence of a HTT oligonucleotide—such oligonucleotide comprises a nucleobase modification at the C position (e.g., see various oligonucleotides in Table 1). In description of oligonucleotides, typically unless otherwise noted, nucleobases, sugars and internucleotidic linkages are non-modified. For example, in various oligonucleotides herein, Aeo, Geo, Teo, m5Ceo are modified as indicated (modified A, G, T or C, which are each 2′-MOE modified; and additionally 5-methyl modification for m5Ceo); C, T, G and A are unmodified deoxyribonucleosides comprising nucleobases C, T, G and A, respectively (e.g., as commonly occurring in natural DNA, no sugar or base modifications); m indicates 2′-OMe modification (e.g., mA is modified A with 2′-OMe; mU is modified U with 2′-OMe; etc.); and each internucleotidic linkage, unless otherwise noted, is independently a natural phosphate linkage (e.g., natural phosphate linkages between . . . Aeom5Ceo . . . ); and each Sp phosphorothioate internucleotidic linkage is represented by * S (or *S); each Rp phosphorothioate internucleotidic linkage is represented by * R (or *R), and a stereorandom phosphorothioate internucleotidic linkage in compositions is represented by *.

In some embodiments, a modified base is optionally substituted adenine, cytosine, guanine, thymine, or uracil, or a tautomer thereof. In some embodiments, a modified nucleobase is a modified adenine, cytosine, guanine, thymine or uracil, modified by one or more modifications by which:
(1) a nucleobase is modified by one or more optionally substituted groups independently selected from acyl, halogen, amino, azide, alkyl, alkenyl, alkynyl, aryl, heteroalkyl, heteroalkenyl, heteroalkynyl, heterocyclyl, heteroaryl, carboxyl, hydroxyl, biotin, avidin, streptavidin, substituted silyl, and combinations thereof,
(2) one or more atoms of a nucleobase are independently replaced with a different atom selected from carbon, nitrogen and sulfur;
(3) one or more double bonds in a nucleobase are independently hydrogenated; or
(4) one or more aryl or heteroaryl rings are independently inserted into a nucleobase.

In some embodiments, a modified nucleobase is a modified nucleobase known in the art, e.g., WO2017/210647. In some embodiments, modified nucleobases are expanded-size nucleobases in which one or more aryl and/or heteroaryl rings, such as phenyl rings, have been added. Certain examples of modified nucleobases, including nucleobase replacements, are described in the Glen Research catalog (Glen Research, Sterling, Va.); Krueger A T et al., Acc. Chem. Res., 2007, 40, 141-150; Kool, E T, Acc. Chem. Res., 2002, 35, 936-943; Benner S. A., et al., Nat. Rev. Genet., 2005, 6, 553-543; Romesberg, F. E., et al., Curr. Opin. Chem. Biol., 2003, 7, 723-733; or Hirao, I., Curr. Opin. Chem. Biol., 2006, 10, 622-627. In some embodiments, an expanded-size nucleobase is an expanded-size nucleobase described in, e.g., WO2017/210647. In some embodiments, modified nucleobases are moieties such as corrin- or porphyrin-derived rings. Certain porphyrin-derived base replacements have been described in, e.g., Morales-Rojas, H and Kool, E T, Org. Lett., 2002, 4, 4377-4380. In some embodiments, a porphyrin-derived ring is a porphyrin-derived ring described in, e.g., WO2017/219647. In some embodiments, a modified nucleobase is a modified nucleobase described in, e.g., WO2017/219647. In some embodiments, a modified nucleobase is fluorescent. Examples of such fluorescent modified nucleobases include phenanthrene, pyrene, stillbene, isoxanthine, isozanthopterin, terphenyl, terthiophene, benzoterthiophene, coumarin, lumazine, tethered stillbene, benzo-uracil, naphtho-uracil, etc., and those described in e.g., WO2017/210647. In some embodiments, a nucleobase or modified nucleobase is selected from: C5-propyne T, C5-propyne C, C5-Thiazole, phenoxazine, 2-thio-thymine, 5-triazolylphenyl-thymine, diaminopurine, and N2-aminopropylguanine.

In some embodiments, a modified nucleobase is selected from 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. In some embodiments, modified nucleobases are tricyclic pyrimidines, such as 1,3-diazaphenoxazine-2-one, 1,3-diazaphenothiazine-2-one or 9-(2-aminoethoxy)-1,3-diazaphenoxazine-2-one (G-clamp). In some embodiments, modified nucleobases are those in which the purine or pyrimidine base is replaced with other heterocycles, for example, 7-deazaadenine, 7-deazaguanosine, 2-aminopyridine or 2-pyridone. In some embodiments, modified nucleobases are those disclosed in U.S. Pat. No. 3,687,808, The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; or in Chapters 6 and 15, Antisense Drug Technology, Crooke S. T., Ed., CRC Press, 2008, 163-166 and 442-443.

In some embodiments, modified nucleobases and methods thereof are those described in US 20030158403, U.S. Pat. Nos. 3,687,808, 4,845,205, 5,130,302, 5,134,066, 5,175,273, 5,367,066, 5,432,272, 5,434,257, 5,457,187, 5,459,255, 5,484,908, 5,502,177, 5,525,711, 5,552,540, 5,587,469, 5,594,121, 5,596,091, 5,614,617, 5,645,985, 5,681,941, 5,750,692, 5,763,588, 5,830,653, or U.S. Pat. No. 6,005,096.

In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One example of a universal base is 3-nitropyrrole.

In some embodiments, nucleosides that can be utilized in provided technologies comprise modified nucleobases and/or modified sugars, e.g., 4-acetylcytidine; 5-(carboxyhydroxylmethyl)uridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

In some embodiments, a nucleobase, e.g., a modified nucleobase comprises one or more biomolecule binding moieties such as e.g., antibodies, antibody fragments, biotin, avidin, streptavidin, receptor ligands, or chelating moieties. In other embodiments, a nucleobase is 5-bromouracil, 5-iodouracil, or 2,6-diaminopurine. In some embodiments, a nucleobase comprises substitution with a fluorescent or biomolecule binding moiety. In some embodiments, a substituent is a fluorescent moiety. In some embodiments, a substituent is biotin or avidin.

Certain examples of nucleobases and related methods are described in U.S. Pat. Nos. 3,687,808, 4,845,205, 5,130,30, 5,134,066, 5,175,273, 5,367,066, 5,432,272, 5,457,187, 5,457,191, 5,459,255, 5,484,908, 5,502,177, 5,525,711, 5,552,540, 5,587,469, 5,594,121, 5,596,091, 5,614,617, 5,681,941, 5,750,692, 6,015,886, 6,147,200, 6,166,197, 6,222,025, 6,235,887, 6,380,368, 6,528,640, 6,639,062, 6,617,438, 7,045,610, 7,427,672, US or U.S. Pat. No. 7,495,088.

In some embodiments, a HTT oligonucleotide comprises a nucleobase, sugar, nucleoside, and/or internucleotidic linkage which is described in any of: Gryaznov, S; Chen, J.-K. J. Am. Chem. Soc. 1994, 116, 3143; Hendrix et al. 1997 Chem. Eur. J. 3: 110; Hyrup et al. 1996 Bioorg. Med. Chem. 4: 5; Jepsen et al. 2004 Oligo. 14: 130-146; Jones et al. J. Org. Chem. 1993, 58, 2983; Koizumi et al. 2003 Nuc. Acids Res. 12: 3267-3273; Koshkin et al. 1998 Tetrahedron 54: 3607-3630; Kumar et al. 1998 Bioo. Med. Chem. Let. 8: 2219-2222; Lauritsen et al. 2002 Chem. Comm. 5: 530-531; Lauritsen et al. 2003 Bioo. Med. Chem. Lett. 13: 253-256; Mesmaeker et al. Angew. Chem., Int. Ed. Engl. 1994, 33, 226; Morita et al. 2001 Nucl. Acids Res. Supp. 1: 241-242; Morita et al. 2002 Bioo. Med. Chem. Lett. 12: 73-76; Morita et al. 2003 Bioo. Med. Chem. Lett. 2211-2226; Nielsen et al. 1997 Chem. Soc. Rev. 73; Nielsen et al. 1997 J. Chem. Soc. Perkins Transl. 1: 3423-3433; Obika et al. 1997 Tetrahedron Lett. 38 (50): 8735-8; Obika et al. 1998 Tetrahedron Lett. 39: 5401-5404; Pallan et al. 2012 Chem. Comm. 48: 8195-8197; Petersen et al. 2003 TRENDS Biotech. 21: 74-81; Rajwanshi et al. 1999 Chem. Commun. 1395-1396; Schultz et al. 1996 Nucleic Acids Res. 24: 2966; Seth et al. 2009 J. Med. Chem. 52: 10-13; Seth et al. 2010 J. Med. Chem. 53: 8309-8318; Seth et al. 2010 J. Org. Chem. 75: 1569-1581; Seth et al. 2012 Bioo. Med. Chem. Lett. 22: 296-299; Seth et al. 2012 Mol. Ther-Nuc. Acids. 1, e47; Seth, Punit P; Siwkowski, Andrew; Allerson, Charles R; Vasquez, Guillermo; Lee, Sam; Prakash, Thazha P; Kinberger, Garth; Migawa, Michael T; Gaus, Hans; Bhat, Balkrishen; et al. From Nucleic Acids Symposium Series (2008), 52(1), 553-554; Singh et al. 1998 Chem. Comm. 1247-1248; Singh et al. 1998 J. Org. Chem. 63: 10035-39; Singh et al. 1998 J. Org. Chem. 63: 6078-6079; Sorensen 2003 Chem. Comm. 2130-2131; Ts'o et al. Ann. N. Y. Acad. Sci. 1988, 507, 220; Van Aerschot et al. 1995 Angew. Chem. Int. Ed. Engl. 34: 1338; Vasseur et al. J. Am. Chem. Soc. 1992, 114, 4006; WO 2007090071; or WO 2016/079181; Feldman et al. 2017 J. Am. Chem. Soc. 139: 11427-11433, Feldman et al. 2017 Proc. Natl. Acad. Sci. USA 114: E6478-E6479, Hwang et al. 2009 Nucl. Acids Res. 37: 4757-4763, Hwang et al. 2008 J. Am. Chem. Soc. 130: 14872-14882, Lavergne et al. 2012 Chem. Eur. J. 18: 1231-1239, Lavergne et al. 2013 J. Am. Chem. Soc. 135: 5408-5419, Ledbetter et al. 2018 J. Am. Chem. Soc. 140: 758-765, Malyshev et al. 2009 J. Am. Chem. Soc. 131: 14620-14621, Seo et al. 2009 Chem. Bio. Chem. 10: 2394-2400, e.g., d3FB, d2Py analogs, d2Py, d3MPy, d4MPy, d5MPy, d34DMPy, d35DMPy, d45DMPy, d5FM, d5PrM, d5SICS, dFEMO, dMMO2, dNaM, dNM01, dTPT3, nucleotides with 2′-azido, 2′-chloro, 2′-amino or arabinose sugars, isocarbostiryl-, napthyl- and azaindole-nucleotides, and modifications and derivatives and functionalized versions thereof, e.g., those in which the sugar comprises a 2′-modification and/or other modification, and dMMO2 derivatives with meta-chlorine, -bromine, -iodine, -methyl, or -propinyl substituents.

In some embodiments, a HTT oligonucleotide comprises a nucleobase or modified nucleobase as described in: WO 2018/022473, WO 2018/098264, WO 2018/223056, WO 2018/223073, WO 2018/223081, WO 2018/237194, WO 2019/032607, WO 2019/055951, and/or WO 2019/075357, U.S. Pat. Nos. 5,552,540, 6,222,025, 6,528,640, 4,845,205, 5,681,941, 5,750,692, 6,015,886, 5,614,617, 6,147,200, 5,457,187, 6,639,062, 7,427,672, 5,459,255, 5,484,908, 7,045,610, 3,687,808, 5,502,177, 5,525,711 6,235,887, 5,175,273, 6,617,438, 5,594,121, 6,380,368, 5,367,066, 5,587,469, 6,166,197, 5,432,272, 7,495,088, 5,134,066, or U.S. Pat. No. 5,596,091, US 2011/0294124, US 2015/0211006, US 2015/0197540, WO 2015/107425, WO 2017/192679, WO 2018/022473, WO 2018/098264, WO 2018/223056, WO 2018/223073, WO 2018/223081, WO 2018/237194, WO 2019/032607, WO 2019/055951, and/or WO 2019/075357, the bases and modified nucleobases of each of which are independently incorporated herein by reference.

In some embodiments, a nucleobase comprises at least one optionally substituted ring which comprises a heteroatom ring atom. In some embodiments, a nucleobase comprises at least one optionally substituted ring which comprises a nitrogen ring atom. In some embodiments, such a ring is aromatic. In some embodiments, a nucleobase is bonded to a sugar through a heteroatom. In some embodiments, a nucleobase is bonded to a sugar through a nitrogen atom. In some embodiments, a nucleobase is bonded to a sugar through a ring nitrogen atom.

In some embodiments, a nucleobase is an optionally substituted purine base residue. In some embodiments, a nucleobase is a protected purine base residue. In some embodiments, a nucleobase is an optionally substituted adenine residue. In some embodiments, a nucleobase is a protected adenine residue. In some embodiments, a nucleobase is an optionally substituted guanine residue. In some embodiments, a nucleobase is a protected guanine residue. In some embodiments, a nucleobase is an optionally substituted cytosine residue. In some embodiments, a nucleobase is a protected cytosine residue. In some embodiments, a nucleobase is an optionally substituted thymine residue. In some embodiments, a nucleobase is a protected thymine residue. In some embodiments, a nucleobase is an optionally substituted uracil residue. In some embodiments, a nucleobase is a protected uracil residue. In some embodiments, a nucleobase is an optionally substituted 5-methylcytosine residue. In some embodiments, a nucleobase is a protected 5-methylcytosine residue.

In some embodiments, a HTT oligonucleotide comprises BrdU, which is a nucleoside unit wherein the nucleobase is BrU

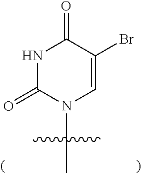 [SMILES: *n1cc(Br)c(=O)[nH]c1=O]

and the sugar is 2-deoxyribose (as widely found in natural DNA)

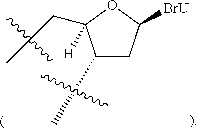

In some embodiments, a HTT oligonucleotide comprises d2AP, DAP and/or dDAP:

d2AP: a nucleoside unit wherein the nucleobase is 2-amino purine

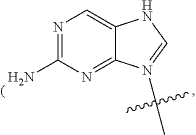

2AP) and wherein the sugar is 2-deoxyribose (as widely found in natural DNA; 2′-deoxy (d))

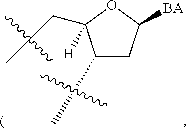

### (BA=2AP);

dDAP: a nucleoside unit wherein the nucleobase is 2,6-diamino purine

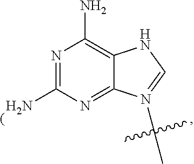

### DAP) and

wherein the sugar is 2-deoxyribose (as widely found in natural DNA; 2′-deoxy (d))

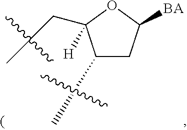

### BA=DAP).

### Additional Chemical Moieties

In some embodiments, an oligonucleotide, e.g., an HTT oligonucleotide, comprises one or more additional chemical moieties. Various additional chemical moieties, e.g., targeting moieties, carbohydrate moieties, lipid moieties, etc. are known in the art and can be utilized in accordance with the present disclosure to modulate properties and/or activities of provided oligonucleotides, e.g., stability, half life, activities, delivery, pharmacodynamics properties, pharmacokinetic properties, etc. In some embodiments, certain additional chemical moieties facilitate delivery of oligonucleotides to desired cells, tissues and/or organs, including but not limited the cells of the central nervous system. In some embodiments, certain additional chemical moieties facilitate internalization of oligonucleotides. In some embodiments, certain additional chemical moieties increase oligonucleotide stability. In some embodiments, the present disclosure provides technologies for incorporating various additional chemical moieties into oligonucleotides.

Reportedly, HTT is expressed in all cells, with the highest concentrations are found in the brain and testes, with moderate amounts in the liver, heart, and lungs. In various embodiments, an additional chemical moiety conjugated to an HTT oligonucleotide allows increased delivery to and/or entrance into a cell in brain, testes, liver, heart, or lungs. HTT protein or mRNA has reportedly been detected in tissues of: adrenal, appendix, bone marrow, brain, colon, duodenum, endometrium, esophagus, fat, gall bladder, heart, kidney, liver, lung, lymph node, ovary, pancreas, placenta, prostate, salivary gland, skin, small intestine, spleen, stomach, testis, thyroid, and urinary bladder. In some embodiments, an HTT oligonucleotide comprises an additional chemical moiety demonstrates increased delivery to and/or activity in an tissue compared to a reference oligonucleotide, e.g., a reference oligonucleotide which does not have the additional chemical moiety but is otherwise identical.

In some embodiments, non-limiting examples of additional chemical moieties include carbohydrate moieties, targeting moieties, etc., which, when incorporated into oligonucleotides, can improve one or more properties. In some embodiments, an additional chemical moiety is selected from: glucose, GluNAc (N-acetyl amine glucosamine) and anisamide moieties. In some embodiments, a provided oligonucleotide can comprise two or more additional chemical moieties, wherein the additional chemical moieties are identical or non-identical, or are of the same category (e.g., carbohydrate moiety, sugar moiety, targeting moiety, etc.) or not of the same category.

In some embodiments, an additional chemical moiety is a targeting moiety. In some embodiments, an additional chemical moiety is or comprises a carbohydrate moiety. In some embodiments, an additional chemical moiety is or comprises a lipid moiety. In some embodiments, an additional chemical moiety is or comprises a ligand moiety for, e.g., cell receptors such as a sigma receptor, an asialoglycoprotein receptor, etc. In some embodiments, a ligand moiety is or comprises an anisamide moiety, which may be a ligand moiety for a sigma receptor. In some embodiments, a ligand moiety is or comprises a GalNAc moiety, which may be a ligand moiety for an asialoglycoprotein receptor.

In some embodiments, a provided oligonucleotide can comprise one or more linkers and additional chemical moieties (e.g., targeting moieties), and/or can be chirally controlled or not chirally controlled, and/or have a bases sequence and/or one or more modifications and/or formats as described herein.

Various linkers, carbohydrate moieties and targeting moieties, including many known in the art, can be utilized in accordance with the present disclosure. In some embodiments, a carbohydrate moiety is a targeting moiety. In some embodiments, a targeting moiety is a carbohydrate moiety.

In some embodiments, a provided oligonucleotide comprises an additional chemical moiety suitable for delivery, e.g., glucose, GluNAc (N-acetyl amine glucosamine), anisamide, or a structure selected from:

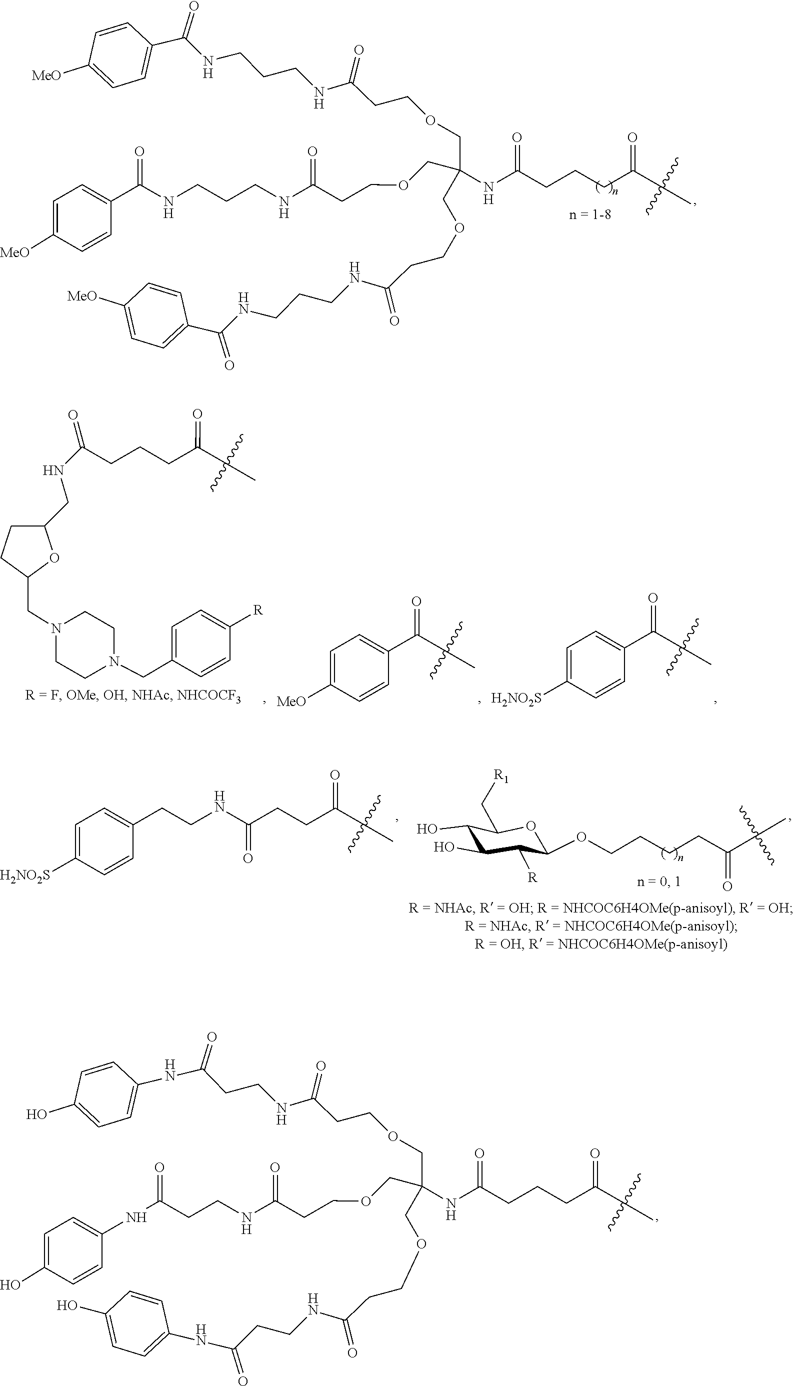

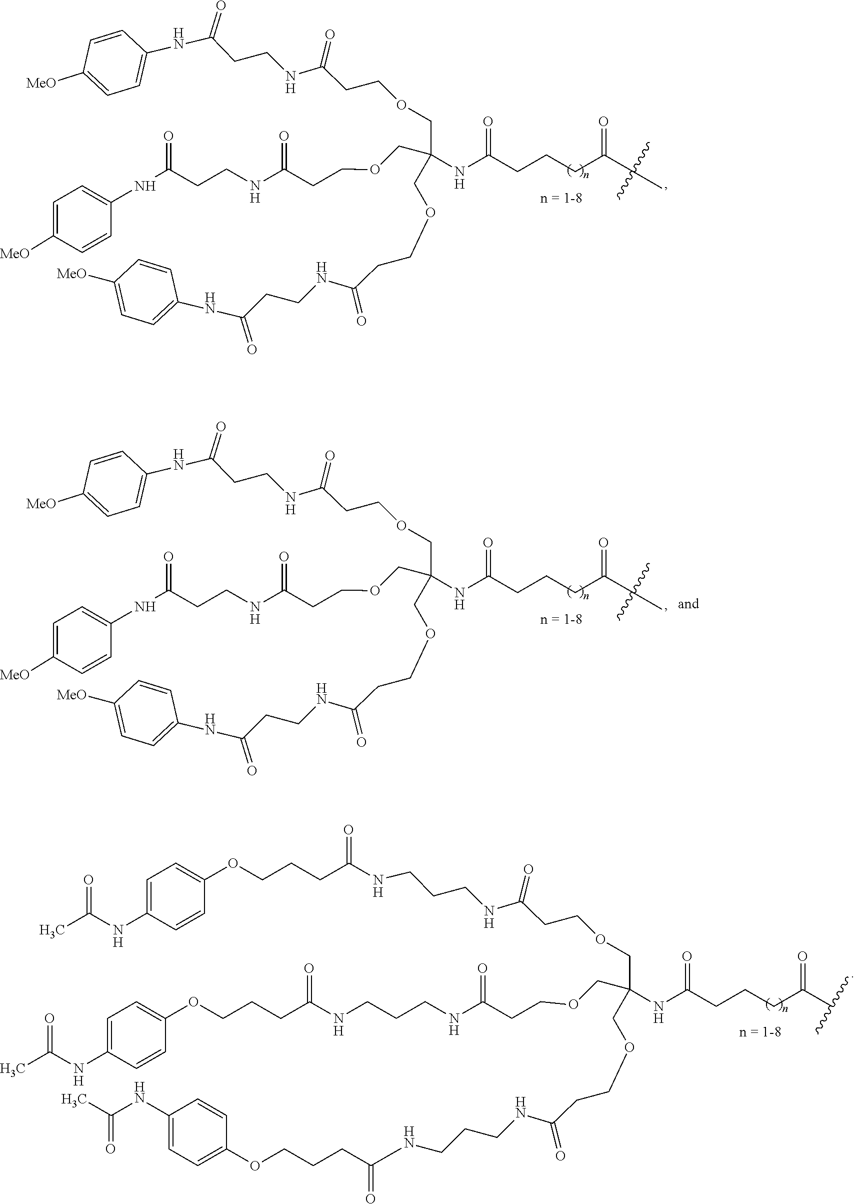

In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8.

In some embodiments, additional chemical moieties are any of ones described in the Examples, including examples of various additional chemical moieties incorporated into various oligonucleotides.

In some embodiments, an additional chemical moiety conjugated to an oligonucleotide is capable of targeting the oligonucleotide to a cell in the central nervous system.

In some embodiments, an additional chemical moiety comprises or is a cell receptor ligand. In some embodiments, an additional chemical moiety comprises or is a protein binder, e.g., one binds to a cell surface protein. Such moieties among other things can be useful for targeted delivery of oligonucleotides to cells expressing the corresponding receptors or proteins. In some embodiments, an additional chemical moiety of a provided oligonucleotide comprises anisamide or a derivative or an analog thereof and is capable of targeting the oligonucleotide to a cell expressing a particular receptor, such as the sigma 1 receptor.

In some embodiments, a provided oligonucleotide is formulated for administration to a body cell and/or tissue expressing its target. In some embodiments, an additional chemical moiety conjugated to an oligonucleotide is capable of targeting the oligonucleotide to a cell.

In some embodiments, an additional chemical moiety is selected from optionally substituted phenyl,

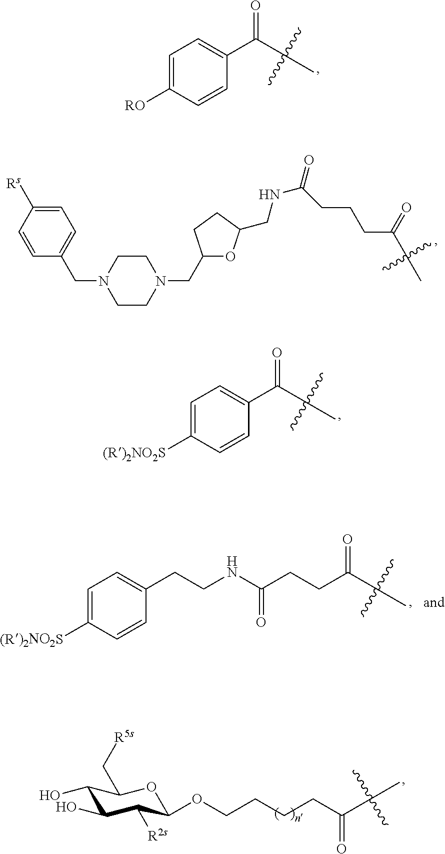

wherein n′ is 1, 2, 3, 4, 5, 6, 7, 8, 9, or 10, and each other variable is as described in the present disclosure. In some embodiments, R^{s} is F. In some embodiments, R^{s} is OMe. In some embodiments, R^{s} is OH. In some embodiments, R^{s} is NHAc. In some embodiments, R^{s} is NHCOCF₃. In some embodiments, R′ is H. In some embodiments, R is H. In some embodiments, R^{2s} is NHAc, and R^{5s} is OH. In some embodiments, R^{2s} is p-anisoyl, and R^{5s} is OH. In some embodiments, R^{2s} is NHAc and R^{5s} is p-anisoyl. In some embodiments, R^{2s} is OH, and R^{5s} is p-anisoyl. In some embodiments, an additional chemical moiety is selected from

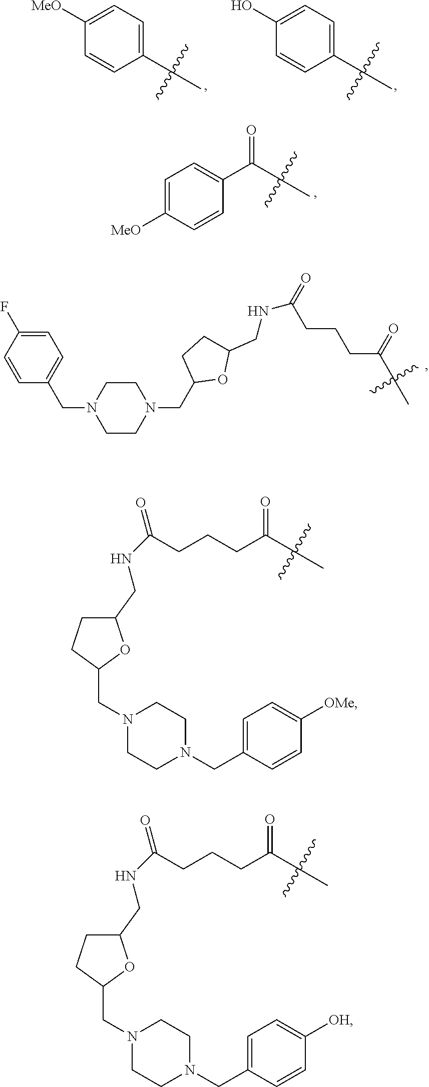 [SMILES: *c1ccc(OC)cc1 ; *c1ccc(O)cc1 ; *C(=O)c1ccc(OC)cc1 ; *C(=O)CCCC(=O)NCC1CCC(CN2CCN(Cc3ccc(F)cc3)CC2)O1 ; *C(=O)CCCC(=O)NCC1CCC(CN2CCN(Cc3ccc(OC)cc3)CC2)O1 ; *C(=O)CCCC(=O)NCC1CCC(CN2CCN(Cc3ccc(O)cc3)CC2)O1]

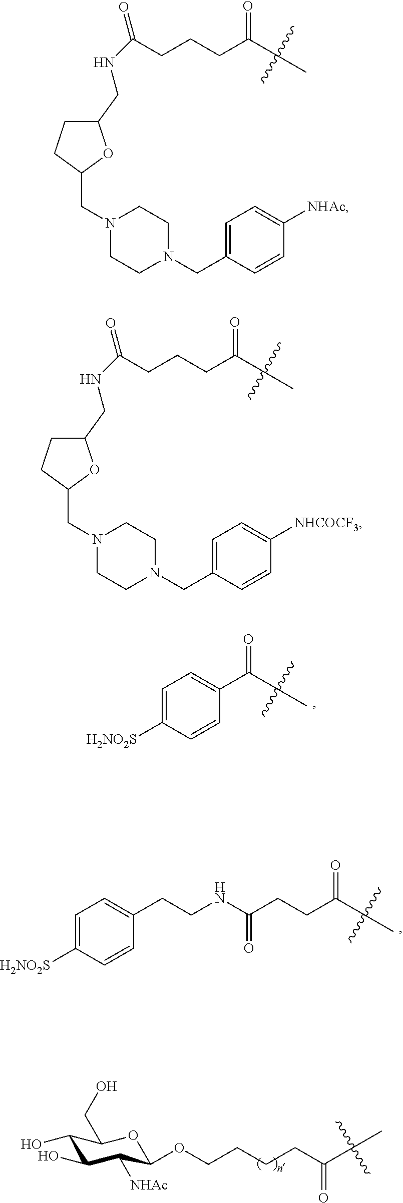

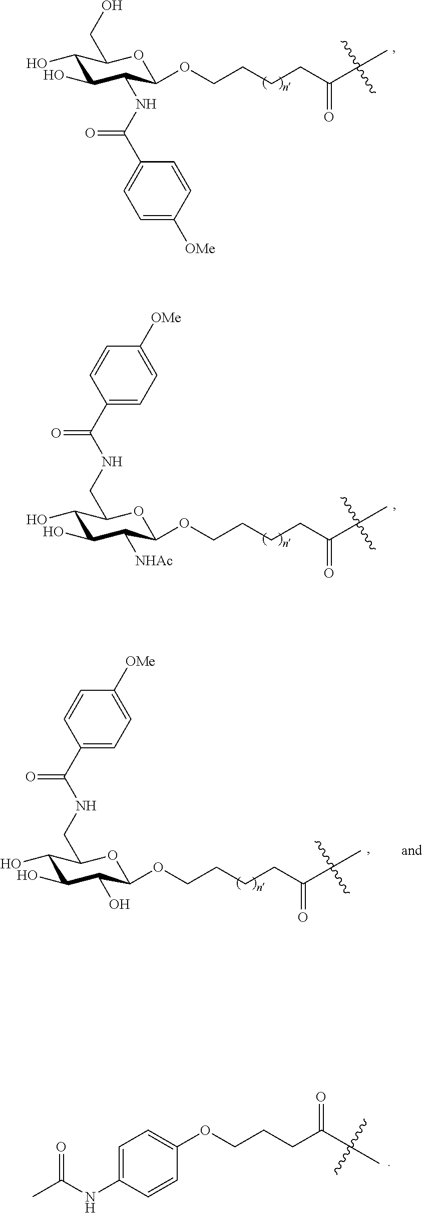

In some embodiments, n′ is 1. In some embodiments, n′ is 0. In some embodiments, n″ is 1. In some embodiments, n″ is 2.

In some embodiments, an additional chemical moiety is or comprises an asialoglycoprotein receptor (ASGPR) ligand.

Without wishing to be bound by any particular theory, the present disclosure notes that ASGPR1 has also been reported to be expressed in the hippocampus region and/or cerebellum Purkinje cell layer of the mouse. http://mouse.brain-map.org/experiment/show/2048

Various other ASGPR ligands are known in the art and can be utilized in accordance with the present disclosure. In some embodiments, an ASGPR ligand is a carbohydrate. In some embodiments, an ASGPR ligand is GalNac or a derivative or an analog thereof. In some embodiments, an ASGPR ligand is one described in Sanhueza et al. J. Am. Chem. Soc., 2017, 139 (9), pp 3528-3536. In some embodiments, an ASGPR ligand is one described in Mamidyala et al. J. Am. Chem. Soc., 2012, 134, pp 1978-1981. In some embodiments, an ASGPR ligand is one described in US 20160207953. In some embodiments, an ASGPR ligand is a substituted-6,8-dioxabicyclo[3.2.1]octane-2,3-diol derivative disclosed in, e.g., US 20160207953. In some embodiments, an ASGPR ligand is one described in, e.g., US 20150329555. In some embodiments, an ASGPR ligand is a substituted-6,8-dioxabicyclo[3.2.1]octane-2,3-diol derivative disclosed e.g., in US 20150329555. In some embodiments, an ASGPR ligand is one described in U.S. Pat. No. 8,877,917, US 20160376585, U.S. Ser. No. 10/086,081, or U.S. Pat. No. 8,106,022. ASGPR ligands described in these documents are incorporated herein by reference. Those skilled in the art will appreciate that various technologies are known in the art, including those described in these documents, for assessing binding of a chemical moiety to ASGPR and can be utilized in accordance with the present disclosure. In some embodiments, a provided oligonucleotide is conjugated to an ASGPR ligand. In some embodiments, a provided oligonucleotide comprises an ASGPR ligand. In some embodiments, an additional chemical moiety comprises an ASGPR ligand is

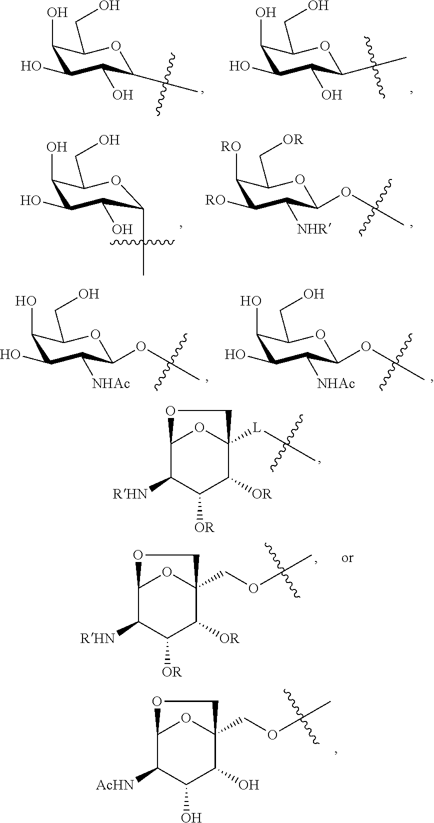

wherein each variable is independently as described in the present disclosure. In some embodiments, R is —H. In some embodiments, R′ is —C(O)R.

In some embodiments, an additional chemical moiety is or comprises

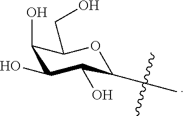

In some embodiments, an additional chemical moiety is or comprises

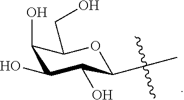

In some embodiments, an additional chemical moiety is or comprises

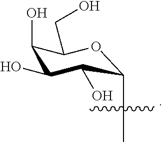

In some embodiments, an additional chemical moiety is or comprises

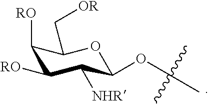

In some embodiments, an additional chemical moiety is or comprises optionally substituted

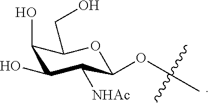

In some embodiments, an additional chemical moiety is or comprises

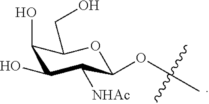

In some embodiments, an additional chemical moiety is or comprises

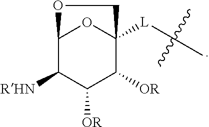

In some embodiments, an additional chemical moiety is or comprises

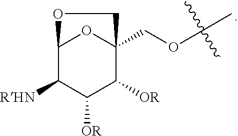

In some embodiments, an additional chemical moiety is or comprises

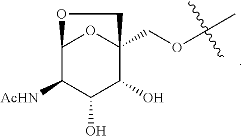 [SMILES: *OC[C@@]12CO[C@@H](O1)[C@H](NC(C)=O)[C@@H](O)[C@H]2O]

In some embodiments, an additional chemical moiety comprises one or more moieties that can bind to, e.g., target cells. For example, in some embodiments, an additional chemistry moiety comprises one or more protein ligand moieties, e.g., in some embodiments, an additional chemical moiety comprises multiple moieties, each of which independently is an ASGPR ligand. In some embodiments, as in Mod 001 and Mod083, an additional chemical moiety comprises three such ligands.

### Mod001:

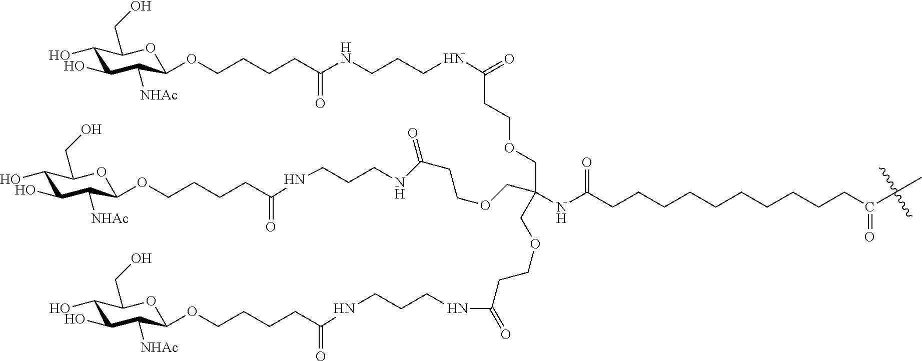

### Mod083:

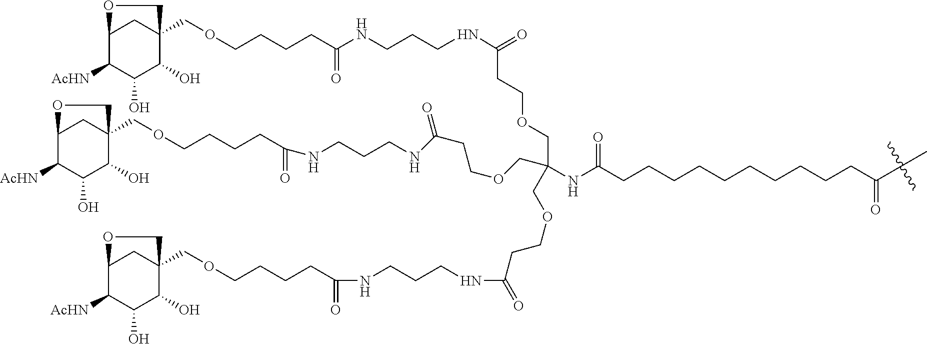 [SMILES: *C(=O)CCCCCCCCCCC(=O)NC(COCCC(=O)NCCCNC(=O)CCCCOC[C@@]12CO[C@@H](C1)[C@H](NC(C)=O)[C@@H](O)[C@H]2O)(COCCC(=O)NCCCNC(=O)CCCCOC[C@@]12CO[C@@H](C1)[C@H](NC(C)=O)[C@@H](O)[C@H]2O)COCCC(=O)NCCCNC(=O)CCCCOC[C@@]12CO[C@@H](C1)[C@H](NC(C)=O)[C@@H](O)[C@H]2O]

In some embodiments, an additional chemical moiety is a Mod group described herein, e.g., in Table 1.

In some embodiments, an additional chemical moiety is or comprises: Mod012 (as a non-limiting example, with —C(O)— connecting to —NH— of a linker such as L001):

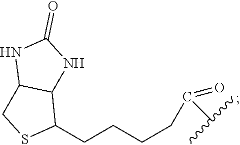 [SMILES: *C(=O)CCCCC1SCC2NC(=O)NC21]

Mod039 (as a non-limiting example, with —C(O)— connecting to —NH— of a linker such as L001 or L004):

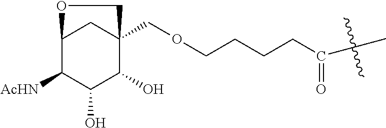 [SMILES: *C(=O)CCCCOC[C@@]12CO[C@@H](C1)[C@H](NC(C)=O)[C@@H](O)[C@H]2O]

Mod062 (as a non-limiting example, with —NH— connecting to —C(O)— of a linker such as L008):

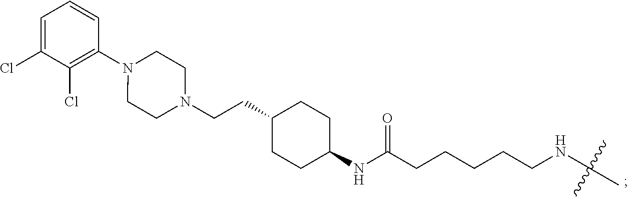 [SMILES: *NCCCCCC(=O)N[C@H]1CC[C@H](CCN2CCN(c3cccc(Cl)c3Cl)CC2)CC1]

Mod085 (as a non-limiting example, with —C(O)— connecting to —NH— of a linker such as L001 or L004):

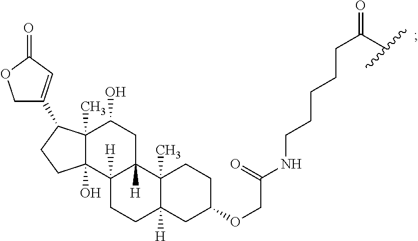

Mod086 (as a non-limiting example, with —C(O)— connecting to —NH— of L001 or L004):

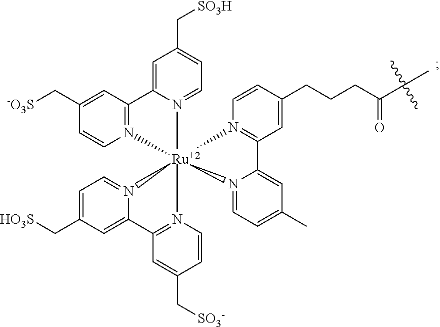

or
Mod094 (as a non-limiting example, bonded to 5′- or 3′-end of an oligonucleotide chain through a phosphate or phosphorothioate):

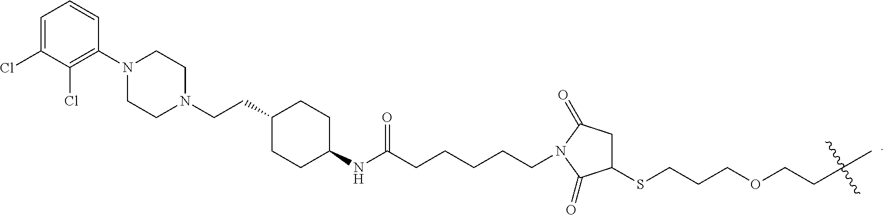 [SMILES: *CCOCCCSC1CC(=O)N(CCCCCC(=O)N[C@H]2CC[C@H](CCN3CCN(c4cccc(Cl)c4Cl)CC3)CC2)C1=O]

In some embodiments, an additional chemical moiety is Mod001. In some embodiments, an additional chemical moiety is Mod083. In some embodiments, an additional chemical moiety, e.g., a Mod group, is directly conjugated (e.g., without a linker) to the remainder of the oligonucleotide. In some embodiments, an additional chemical moiety is conjugated via a linker to the remainder of the oligonucleotide. In some embodiments, additional chemical moieties, e.g., Mod groups, may be directly connected, and/or via a linker, to nucleobases, sugars and/or internucleotidic linkages of oligonucleotides. In some embodiments, Mod groups are connected, either directly or via a linker, to sugars. In some embodiments, Mod groups are connected, either directly or via a linker, to 5′-end sugars. In some embodiments, Mod groups are connected, either directly or via a linker, to 5′-end sugars via 5′ carbon. For examples, see various oligonucleotides in Table 1. In some embodiments, Mod groups are connected, either directly or via a linker, to 3′-end sugars. In some embodiments, Mod groups are connected, either directly or via a linker, to 3′-end sugars via 3′ carbon. In some embodiments, Mod groups are connected, either directly or via a linker, to nucleobases. In some embodiments, Mod groups are connected, either directly or via a linker, to internucleotidic linkages. For example, in some embodiments, an additional chemical moiety can be connected to a nucleobase:

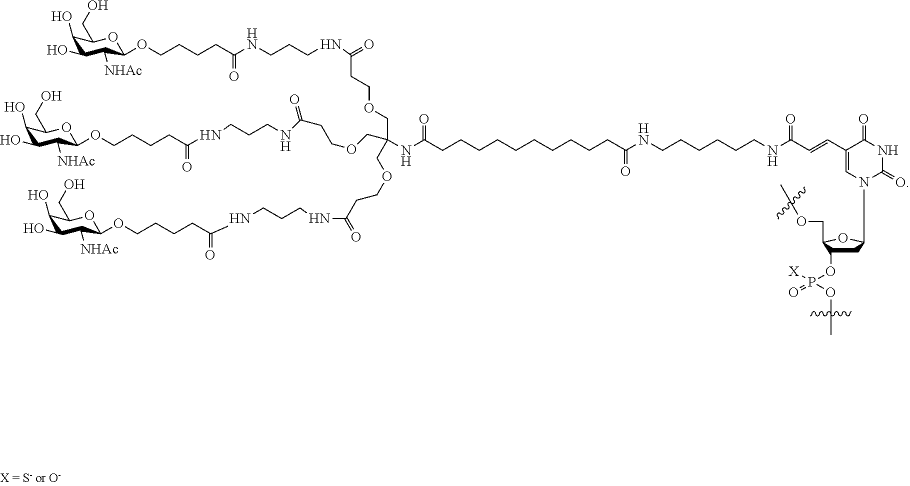

Certain additional chemical moieties (e.g., lipid moieties, targeting moieties, carbohydrate moieties) and linkers for connecting additional chemical moieties to oligonucleotide chains are described in WO 2017/062862, WO 2018/067973, WO 2017/160741, WO 2017/192679, WO 2017/210647, or WO 2018/098264, the additional chemical moieties and linkers of each of which are independently incorporated herein by reference, and can be utilized in accordance with the present disclosure. In some embodiments, an additional chemical moiety is digoxigenin or biotin or a derivative thereof.

In some embodiments, an additional chemical moiety is one described in WO 2012/030683. In some embodiments, a provided oligonucleotide comprise a chemical structure (e.g., a linker, lipid, solubilizing group, and/or targeting ligand) described in WO 2012/030683.

In some embodiments, a provide oligonucleotide comprises an additional chemical moiety and/or a modification (e.g., of nucleobase, sugar, internucleotidic linkage, etc.) described in: U.S. Pat. Nos. 5,688,941; 6,294,664; 6,320,017; 6,576,752; 5,258,506; 5,591,584; 4,958,013; 5,082,830; 5,118,802; 5,138,045; 6,783,931; 5,254,469; 5,414,077; 5,486,603; 5,112,963; 5,599,928; 6,900,297; 5,214,136; 5,109,124; 5,512,439; 4,667,025; 5,525,465; 5,514,785; 5,565,552; 5,541,313; 5,545,730; 4,835,263; 4,876,335; 5,578,717; 5,580,731; 5,451,463; 5,510,475; 4,904,582; 5,082,830; 4,762,779; 4,789,737; 4,824,941; 4,828,979; 5,595,726; 5,214,136; 5,245,022; 5,317,098; 5,371,241; 5,391,723; 4,948,882; 5,218,105; 5,112,963; 5,567,810; 5,574,142; 5,578,718; 5,608,046; 4,587,044; 4,605,735; 5,585,481; 5,292,873; 5,552,538; 5,512,667; 5,597,696; 5,599,923; 7,037,646; 5,587,371; 5,416,203; 5,262,536; 5,272,250; or 8,106,022.

In some embodiments, an additional chemical moiety, e.g., a Mod, is connected via a linker. Various linkers are available in the art and may be utilized in accordance with the present disclosure, for example, those utilized for conjugation of various moieties with proteins (e.g., with antibodies to form antibody-drug conjugates), nucleic acids, etc. Certain useful linkers are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194, the linker moieties of each which are independently incorporated herein by reference. In some embodiments, a linker is, as non-limiting examples, L001, L004, L009 or L010. In some embodiments, an oligonucleotide comprises a linker, but not an additional chemical moiety other than the linker. In some embodiments, an oligonucleotide comprises a linker, but not an additional chemical moiety other than the linker, wherein the linker is L001, L004, L009, or L010.

L003

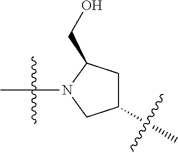

linker. In some embodiments, it is connected to Mod, if any (if no Mod, —H), through its amino group, and the 5′-end or 3′-end of an oligonucleotide chain e.g., via a linkage (e.g., a phosphate linkage (O or PO) or a phosphorothioate linkage (can be either not chirally controlled or chirally controlled (Sp or Rp))).

L009: —CH₂CH₂CH₂—. In some embodiments, when L009 is present at the 5′-end of an oligonucleotide without a Mod, one end of L009 is connected to —OH and the other end connected to a 5′-carbon of the oligonucleotide chain e.g., via a linkage (e.g., a phosphate linkage (O or PO) or a phosphorothioate linkage (can be either not chirally controlled or chirally controlled (Sp or Rp))).

L010:

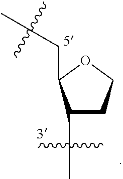

In some embodiments, when L010 is present at the 5′-end of an oligonucleotide without a Mod, the 5′-carbon of L010 is connected to —OH and the 3′-carbon connected to a 5′-carbon of the oligonucleotide chain e.g., via a linkage (e.g., a phosphate linkage (O or PO) or a phosphorothioate linkage (can be either not chirally controlled or chirally controlled (Sp or Rp))).

Non-limiting examples of oligonucleotides, e.g., HTT oligonucleotides, which comprise an additional chemical moiety include: WV-10483, WV-10484, WV-10485, WV-10486, WV-10631, WV-10632, WV-10633, WV-10640, WV-10641, WV-10642, WV-10643, WV-10644, WV-11569, WV-11570, WV-11571, and WV-20213.

### Oligonucleotide Multimers

In some embodiments, the present disclosure provides multimers of oligonucleotides. In some embodiments, at least one of the monomer is a provided oligonucleotide. In some embodiments, at least one of the monomer is an HTT oligonucleotide. In some embodiments, a multimer is a multimer of the same oligonucleotides. In some embodiments, a multimer is a multimer of structurally different oligonucleotides. In some embodiments, a multimer is a multimer of oligonucleotides whose base sequences are not the same. In some embodiments, each oligonucleotide of a multimer performs its functions independently through its own pathways, e.g., RNA interference (RNAi), RNase H dependent, etc. In some embodiments, provided oligonucleotides exist in an oligomeric or polymeric form, in which one or more oligonucleotide moieties are linked together by linkers, through nucleobases, sugars, and/or internucleotidic linkages of the oligonucleotide moieties.

In some embodiments, a multimer comprises 2 oligonucleotides. In some embodiments, a multimer comprises 3 oligonucleotides. In some embodiments, a multimer comprises 4 oligonucleotides. In some embodiments, a multimer comprises 5 oligonucleotides. In some embodiments, a multimer comprises 2 HTT oligonucleotides. In some embodiments, a multimer comprises 3 HTT oligonucleotides. In some embodiments, a multimer comprises 4 HTT oligonucleotides. In some embodiments, a multimer comprises 5 HTT oligonucleotides.

In some embodiments, a multimer has a multimer structure described in WO 2017/062862, WO 2018/067973, WO 2017/160741, WO 2017/192679, WO 2017/210647, or WO 2018/098264, the multimer of each of which is independently incorporated herein by reference.

### Production of Oligonucleotides and Compositions

Various methods can be utilized for production of oligonucleotides and compositions and can be utilized in accordance with the present disclosure. For example, traditional phosphoramidite chemistry can be utilized to prepare stereorandom oligonucleotides and compositions, and certain reagents and chirally controlled technologies can be utilized to prepare chirally controlled oligonucleotide compositions, e.g., as described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194, the reagents and methods of each of which is incorporated herein by reference.

In some embodiments, chirally controlled/stereoselective preparation of oligonucleotides and compositions thereof comprise utilization of a chiral auxiliary, e.g., as part of monomeric phosphoramidites. Examples of such chiral auxiliary reagents and phosphoramidites are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194, the chiral auxiliary reagents and phosphoramidites of each of which are independently incorporated herein by reference. In some embodiments, a chiral auxiliary is

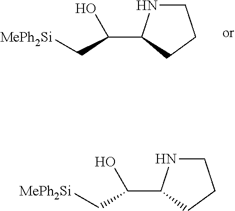

(DPSE chiral auxiliaries). In some embodiments, a chiral auxiliary is

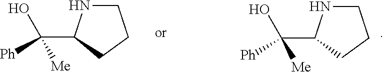 [SMILES: C[C@@](O)(c1ccccc1)[C@@H]1CCCN1 ; C[C@](O)(c1ccccc1)[C@H]1CCCN1]

In some embodiments, a chiral auxiliary is

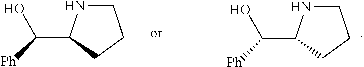 [SMILES: O[C@H](c1ccccc1)[C@@H]1CCCN1 ; O[C@@H](c1ccccc1)[C@H]1CCCN1]

In some embodiments, a chiral auxiliary is

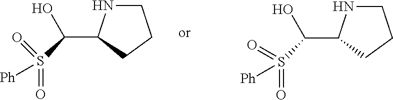 [SMILES: O=S(=O)(c1ccccc1)[C@@H](O)[C@@H]1CCCN1 ; O=S(=O)(c1ccccc1)[C@H](O)[C@H]1CCCN1]

(PSM chiral auxiliaries).

In some embodiments, chirally controlled preparation technologies, including oligonucleotide synthesis cycles, reagents and conditions are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, or WO 2018/098264, the oligonucleotide synthesis methods, cycles, reagents and conditions of each of which are independently incorporated herein by reference. In some embodiments, a useful oligonucleotide synthesis cycle using DPSE chiral auxiliaries is depicted below, wherein each of BA₁, BA₂ and BA₃ is independently BA, R^{LP} is -L-R¹, and each other variables is independently as described in the present disclosure.

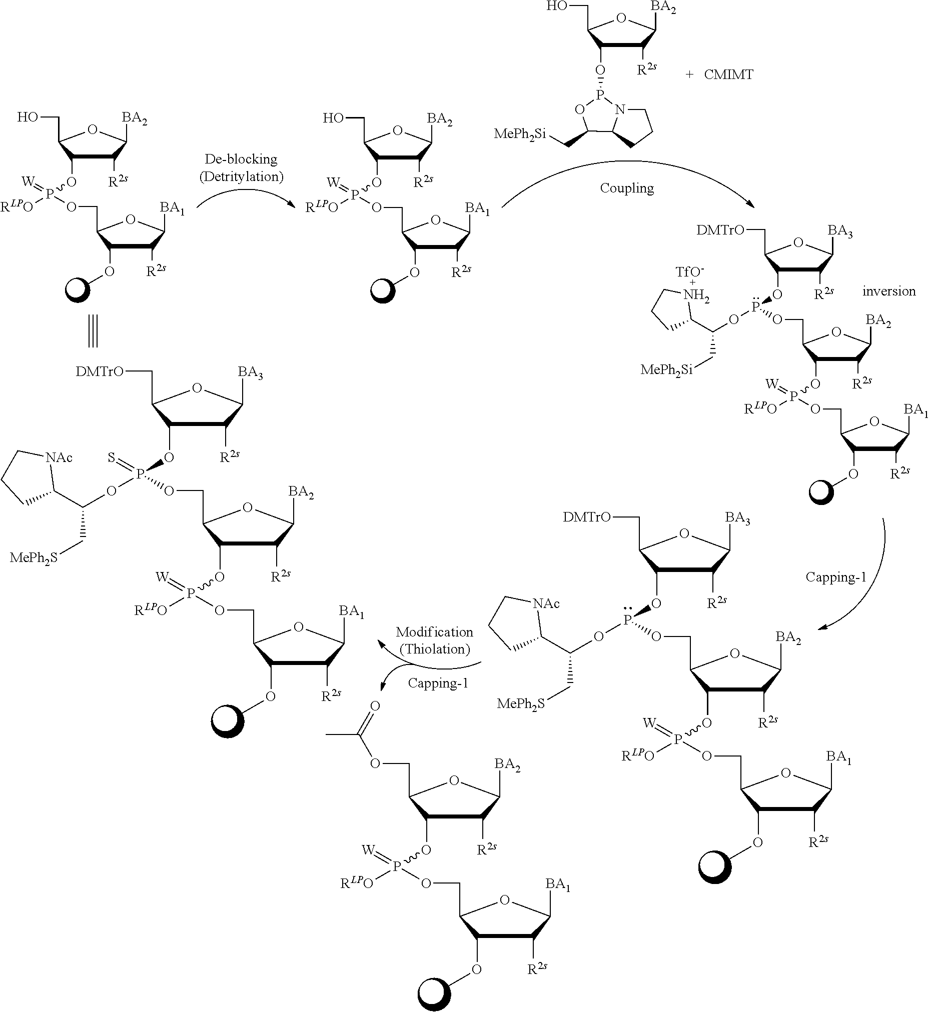

Once synthesized, provided oligonucleotides and compositions are typically further purified. Suitable purification technologies are widely known and practiced by those skilled in the art including but not limited to those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194, the purification technologies of each of which are independently incorporated herein by reference.

In some embodiments, a cycle comprises or consists of coupling, capping, modification and deblocking. In some embodiments, a cycle comprises or consists of coupling, capping, modification, capping and deblocking. These steps are typically performed in the order they are listed, but in some embodiments, as appreciated by those skilled in the art, the order of certain steps, e.g., capping and modification, may be altered. If desired, one or more steps may be repeated to improve conversion, yield and/or purity as those skilled in the art often perform in syntheses. For example, in some embodiments, coupling may be repeated; in some embodiments, modification (e.g., oxidation to install ═O, sulfurization to install ═S, etc.) may be repeated; in some embodiments, coupling is repeated after modification which can convert a P(III) linkage to a P(V) linkage which can be more stable under certain circumstances, and coupling is routinely followed by modification to convert newly formed P(III) linkages to P(V) linkages. In some embodiments, when steps are repeated, different conditions may be employed (e.g., concentration, temperature, reagent, time, etc.).

Technologies for formulating provided oligonucleotides and/or preparing pharmaceutical compositions, e.g., for administration to subjects via various routes, are readily available in the art and can be utilized in accordance with the present disclosure, e.g., those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194 and references cited therein.

### Biological Applications

As appreciated by those skilled in the art, oligonucleotides are useful for multiple purposes. In some embodiments, provided technologies (e.g., oligonucleotides, compositions, methods, etc.) are useful for reducing levels and/or activities of various transcripts (e.g., RNA) and/or products encoded thereby (e.g., proteins). In some embodiments, provided technologies reduce levels and/or activities RNA, e.g., HTT RNA transcripts. In some embodiments, provided oligonucleotides and compositions provide improved knockdown of transcripts, e.g., HTT transcripts, compared to a reference condition selected from the group consisting of absence of the oligonucleotide or composition, presence of a reference oligonucleotide or composition, and combinations thereof. Certain example applications and/or methods for using and making various oligonucleotides are described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194.

For example, in some embodiments, a provided oligonucleotide is an HTT oligonucleotide capable of mediating a decrease in the expression, activity and/or level of an HTT gene product. An improvement mediated by an HTT oligonucleotide can be an improvement of any desired biological functions, including but not limited to treatment and/or prevention of an HTT-related disorder or a symptom thereof.

In some embodiments, a provided compound, e.g., oligonucleotide, and/or compositions thereof, can modulate activities and/or functions of a target gene. In some embodiments, a target gene is a gene with respect to which expression and/or activity of one or more gene products (e.g., RNA and/or protein products) are intended to be altered. In many embodiments, a target gene is intended to be inhibited. Thus, when an oligonucleotide as described herein acts on a particular target gene, presence and/or activity of one or more gene products of that gene are altered when the oligonucleotide is present as compared with when it is absent. In some embodiments, a target gene is HTT.

In some embodiments, a target sequence is a sequence of a gene or a transcript thereof to which an oligonucleotide hybridizes. In some embodiments, a target sequence is fully complementary or substantially complementary to a sequence of an oligonucleotide, or of consecutive residues therein (e.g., an oligonucleotide includes a target-binding sequence that is an exact complement of a target sequence). In some embodiments, a small number of differences/mismatches is tolerated between (a relevant portion of) an oligonucleotide and its target sequence. In many embodiments, a target sequence is present within a target gene. In many embodiments, a target sequence is present within a transcript (e.g., an mRNA and/or a pre-mRNA) produced from a target gene. In some embodiments, a target sequence is an HTT target sequence which is a sequence of an HTT gene or a transcript thereof to which an HTT oligonucleotide hybridizes.

In some embodiments, provided oligonucleotides and compositions are useful for treating various conditions, disorders or diseases, by reducing levels and/or activities of transcripts and/or products encoded thereby that are associated with the conditions, disorders or diseases. In some embodiments, the present disclosure provides methods for preventing or treating a condition, disorder or disease, comprising administering to a subject susceptible to or suffering from a condition, disorder or disease a provided oligonucleotide or composition thereof. In some embodiments, a provided oligonucleotide or oligonucleotides in a provided composition are of a base sequence that is or is complementary to a portion of a transcript, which transcript is associated with a condition, disorder or disease. In some embodiments, a base sequence is such that it selectively bind to a transcript, e.g., an HTT transcript, associated with a condition, disorder or disease over other transcripts that are not associated with the same condition, disorder or disease. In some embodiments, a condition, disorder or disease is associated with HTT.

In some embodiments, in a method of treating a disease by administering a composition comprising a plurality of oligonucleotides sharing a common base sequence, which base sequence is complementary to a target sequence in a target transcript, the present disclosure provides an improvement that comprises administering as the oligonucleotide composition a chirally controlled oligonucleotide composition as described in the present disclosure, characterized in that, when it is contacted with the target transcript in a knockdown system, knockdown of the transcript is improved relative to that observed under a reference condition selected from the group consisting of absence of the composition, presence of a reference composition, and combinations thereof. In some embodiments, a reference composition is a racemic preparation of oligonucleotides of the same sequence or constitution. In some embodiments, a target transcript is an HTT transcript.

In some embodiments, provided oligonucleotides can bind to a transcript, and improve knockdown of the transcript (e.g., an HTT RNA). In some embodiments, HTT oligonucleotides improve knockdown, e.g., HTT knockdown, with efficiency greater than a comparable oligonucleotide under one or more suitable conditions.

In some embodiments, an oligonucleotide, e.g., an HTT oligonucleotide, or a composition thereof is capable of mediating a decrease in the expression or level of a target gene, e.g., HTT, or a gene product thereof at an oligonucleotide, e.g., an HTT oligonucleotide, concentration of 1 nm or less in a cell in vitro. In some embodiments, an oligonucleotide, e.g., an HTT oligonucleotide, or a composition thereof is capable of mediating a decrease in the expression or level of a target gene, e.g., HTT, or a gene product thereof at an oligonucleotide, e.g., an HTT oligonucleotide, concentration of 5 nm or less in a cell in vitro. In some embodiments, an oligonucleotide, e.g., an HTT oligonucleotide, or a composition thereof is capable of mediating a decrease in the expression or level of a target gene, e.g., HTT, or a gene product thereof at an oligonucleotide, e.g., an HTT oligonucleotide, concentration of 10 nm or less in a cell in vitro.

In some embodiments, activity of a provided oligonucleotide or oligonucleotide composition may be assessed by IC50 which is the inhibitory concentration to decrease expression or level of a target gene or a gene product thereof by 50% in a suitable condition, e.g., cell-based in vitro assays. In some embodiments, provided oligonucleotides have an IC50 no more than 0.001, 0.01, 0.1, 0.5, 1, 2, 5, 10, 50, 100, 200, 500 or 1000 nM. In some embodiments, an oligonucleotide, e.g., an HTT oligonucleotide, has an IC50 of no more than about 10 nM in a cell(s) in vitro. In some embodiments, an oligonucleotide, e.g., an HTT oligonucleotide, has an IC50 of no more than about 5 nM in a cell(s) in vitro. In some embodiments, an oligonucleotide, e.g., an HTT oligonucleotide, has an IC50 of no more than about 2 nM in a cell(s) in vitro. In some embodiments, an oligonucleotide, e.g., an HTT oligonucleotide, has an IC50 of no more than about 1 nM in a cell(s) in vitro. In some embodiments, an oligonucleotide, e.g., an HTT oligonucleotide, has an IC50 of no more than about 0.5 nM in a cell(s) in vitro. In some embodiments, an oligonucleotide, e.g., an HTT oligonucleotide, has an IC50 of no more than about 0.1 nM in a cell(s) in vitro. In some embodiments, an oligonucleotide, e.g., an HTT oligonucleotide, has an IC50 of no more than about 0.01 nM in a cell(s) in vitro. In some embodiments, an oligonucleotide, e.g., an HTT oligonucleotide, has an IC50 of no more than about 0.001 nM in a cell(s) in vitro.

In some embodiments, the pattern of stereochemistry of a provided HTT oligonucleotide comprises a pattern of stereochemistry described herein or any portion thereof. In some embodiments, an oligonucleotide comprises a pattern of stereochemistry described herein and is capable of directing RNase H-mediated knockdown. In some embodiments, a provided HTT oligonucleotide comprises a pattern of stereochemistry described herein and is capable of directing RNase H-mediated HTT knockdown.

In some embodiments, a provided HTT oligonucleotide comprises a modification or pattern of modification described herein. In some embodiments, a provided HTT oligonucleotide comprises a pattern of modification described herein and is capable of directing RNase H-mediated HTT knockdown. In some embodiments, a modification or pattern of modification is a modification or pattern of modification of sugar modifications, e.g., modifications at the 2′ position of sugars (e.g., 2′-F, 2′-OMe, 2′-MOE, etc.).

### Targeting a Huntington's Disease-associated Allele by Targeting an Associated SNP

Among other things, oligonucleotides of the present disclosure can provide high specificity. For example, in some embodiments, an oligonucleotide targeting HTT is capable of mediating allele-specific knockdown, wherein the mutant, HD-associated allele of HTT (or a gene product thereof) is knocked down to a greater extent than an allele that is not associated or less associated, e.g., a wild-type allele. In some embodiments, a HD-associated allele comprises expanded CAG repeats. In some embodiments, allele-specific knockdown is achieved with an HTT oligonucleotide which does not target the CAG region of the disease-associated HTT allele, but rather another genetic locus on the same genetic material. As demonstrated herein, a nucleic acid therapy can be designed which targets a transcript, e.g., mRNA, with a mutation, but does not directly target the site of the mutation. Instead, a nucleic acid therapy can target another genetic locus, such as a single nucleotide polymorphism (SNP), which is on the same transcript, e.g., mRNA, as the mutation (e.g., expanded CAG in HTT).

In some embodiments, for the treatment of an autosomal dominant disease, such as Huntington's disease (HD), in which one mutated copy of a gene is sufficient to cause disease, selectively targeting transcripts, e.g., mRNA, corresponding to the disease-causing allele is preferred. In some embodiments, a strategy to achieve this end involves using an oligonucleotide, e.g., an HTT oligonucleotide, capable of targeting a SNP, e.g., an HTT SNP, where one variant of a SNP associates with a disease-causing mutation at high frequency.

In some embodiments, a SNP is a variation in a single nucleotide that occurs at a specific position in the genome, where each variation is present to some appreciable degree within a population (e.g., >1%). In some embodiments, the terms “single nucleotide polymorphism” and “SNP”, as used herein, refer to a single nucleotide variation among genomes of individuals of the same species. For example, at a specific base position in the human genome, the base C may appear in most individuals, but in an appreciable minority of individuals, the position is occupied by base A. There is an SNP at this specific base position, and the two possible nucleotide variations—C or A—are said to be alleles (or variants or isoforms) for this base position. In some embodiments, there are only two different alleles. In some embodiments, a SNP is triallelic in which three different base variations may coexist within a population. Hodgkinson et al. 2009 Genetics 1. doi:10.4172/2157-7145.1000107. In some embodiments, a SNP may be a single nucleotide deletion or insertion. In general, SNPs may occur relatively frequently in genomes and contribute to genetic diversity. In some embodiments, the location of a SNP is flanked by highly conserved sequences. In some embodiments, an individual may be homozygous or heterozygous for an allele at each SNP site. A heterozygous SNP allele can be a differentiating polymorphism. A SNP may be targeted, optionally with selectivity as demonstrated herein, with an oligonucleotide.

Large collections of confirmed and annotated SNPs are publicly available (e.g., The SNP Consortium, National Center for Biotechnology Information, Cold Spring Harbor Laboratory) [Sachidanandam et al. 2001 Nature 409: 928-933; The 1000 Genomes Project Consortium 2010 Nature 467: 1061-73 and Corrigendum; Kay et al. 2015 Mol. Ther. 23: 1759-1771].

Many SNPs in the HTT gene (e.g., HTT SNPs) are reportedly associated with disease chromosomes and have strong linkage associations with the deleterious, HD-associated CAG expansion. Many SNPs highly associated with CAG expansion do not segregate independently and are in Linkage Disequilibrium with each other. Among other things, the present disclosure recognizes that strong association between specific HTT SNPs and CAG expanded chromosomes provides an attractive therapeutic opportunity for the treatment of Huntington's Disease, e.g., through antisense therapy. Furthermore, the association of specific SNPs combined with high rates of heterozygosity in HD patients provides suitable targets for allele-specific knockdown of the mutant gene product.

In some embodiments, one variant of an HTT SNP may be more commonly associated with (e.g., on the same chromosome as, or in-phase with) the deleterious CAG expansion. In some embodiments, a variant of a SNP is also designated an isoform of a SNP. In some embodiments, an HTT oligonucleotide targets a variant of a SNP which is in phase (e.g., on the same allele or on the same chromosome) as the deleterious CAG expanion, and the HTT oligonucleotide is capable of mediating allele-specific inhibition (or suppression), wherein the level, expression and/or activity of the mutant HTT allele (comprising the CAG expression) is decreased preferentially relative to the level, expression and/or activity of the wild-type HTT allele (which does not comprise the CAG expansion).

In some embodiments, prior to treating a subject with an HTT oligonucleotide which targets a particular variant of a particular SNP and which is capable of mediating allele-specific knockdown of the mutant HTT, a genetic analysis of the subject is performed to determine which variant of the targeted SNP is on the same chromosome as the deleterious CAG expansion. In some embodiments, the broad category of methods for determining if a particular SNP isoform is on the same chromosome as (e.g., on the same allele as or in phase with) the CAG expansion is designated phasing. Various methods of phasing are described herein and in a later section.

At a given gene locus on a pair of autosomal chromosomes, a diploid organism (e.g., a human being) inherits one allele of the gene from the mother and another allele of the gene from the father. At a heterozygous gene locus, two parents contribute different alleles (e.g., one A and one a). Without additional processing, it may be impossible to tell which parent contributed which allele. Such genotype data that is not attributed to a particular parent is referred to as unphased genotype data. Typically, initial genotype readings obtained from genotyping chips are often in an unphased form.

Many sequencing procedures can reveal that an individual has sequence variability at particular positions. For example, at one position (e.g., a SNP), the individual may have a C in one copy of the gene and a G on the other. For a separate position (e.g., a different SNP), the individual may have a A in one copy and a U in the other. Because many sequencing techniques involve fragmentation of the nucleic acid template, depending on the sequencing technique used, it may not be possible to determine, for example, if the C and A or C and U are on the same chromosome. Phasing information will provide information on the arrangement of the different alleles on the different chromosomes.

As noted by Laver et al., phasing is also important in pharmacogenetics, transplant HLA typing and disease association mapping. Laver et al. 2016 Nature Scientific Reports 6:21746 DOI: 10.1038/srep21746. Phasing of allelic variants is important for clinical interpretation of the genome, population genetic analysis, and functional genomic analysis of allelic activity. The phasing of rare and de novo variants is crucial for identifying putative causal variants in clinical genetics applications, for example by distinguishing compound heterozygotes from two variants on the same allele.

In some embodiments, an HTT oligonucleotide targets a portion of an HTT transcript, e.g., mRNA, comprising a position of a SNP. Many HTT SNPs are known in the art.

In some embodiments of a method for treatment of Huntington's Disease, a patient is afflicted with Huntington's Disease characterized by an expanded CAG repeat in one allele of the HTT gene, and the patient is administered a therapeutically effective amount of an HTT oligonucleotide, wherein the HTT targets an HTT SNP (e.g., a portion of an HTT mRNA comprising the position of a SNP), wherein the SNP is on the same chromosome (e.g., in the same phase) as the expanded CAG repeat.

In some embodiments, an oligonucleotide comprises a sequence that is complementary to an SNP allele associated with a condition, disorder or disease. In some embodiments, an HTT oligonucleotide targets an HTT site which is selected from any of the following SNPs: rs362267, rs362268, rs362272, rs362273, rs362275, rs362302, rs362303, rs362304, rs362305, rs362306, rs362307, rs362308, rs362331, rs362336, rs363075, rs363088, rs363125, rs1065746, rs1557210, rs2024115, rs2298969, rs2530595, rs3025805, rs3025806, rs4690072, rs4690074, rs6844859, rs7685686, rs17781557, and rs35892913.

In some embodiments, an HTT oligonucleotide targets an HTT site which is selected from any of the following SNPs: rs362267, rs362268, rs362272, rs362273, rs362275, rs362302, rs362303, rs362304, rs362305, rs362306, rs362307, rs362308, rs362331, rs362336, rs363075, rs363088, rs363125, rs1065746, rs1557210, rs2024115, rs2298969, rs3025805, rs3025806, rs4690072, rs4690074, rs6844859, rs7685686, rs113407847, rs17781557, and rs35892913.

In some embodiments, a targeted SNP is rs362268, rs362306, rs362307, rs362331, rs2530595, or rs7685686. In some embodiments, a targeted SNP is rs362307, rs7685686, rs362268 or rs362306. In some embodiments, a targeted SNP is rs362307. In some embodiments, a targeted SNP is rs7685686. In some embodiments, a targeted SNP is not rs7685686. In some embodiments, a targeted SNP is rs362268.

In some embodiments, a targeted HTT SNP is: rs362268, rs362272, rs362273, rs362306, rs362307, rs362331, rs363099, rs2530595, rs2830088, rs7685686, or rs113407847, or any HTT SNP disclosed herein.

In some embodiments, an HTT oligonucleotide targets an HTT site which is selected from any of the following SNPs (wherein one variant of the SNP is noted after the SNP number): rs10015979_G, rs1006798_A, rs10488840_G, rs108850_C, rs11731237_T, rs1263309_T, rs16843804_C, rs2024115_A, rs2285086_A, rs2298967_T, rs2298969_A, rs2798235_G, rs2798296_G, rs2857936_C, rs3095074_G, rs3121417_G, rs3121419_C, rs3129322_T, rs34315806_C, rs362271_G, rs362272_G, rs362273_A, rs362275_C, rs362296_C, rs362303_C, rs362306_G, rs362307_T rs362310_C, rs362331_T, rs363064_C, rs363072_A, rs363080_C, rs363088_A, rs363092_C, rs363096_T, rs363099_C, rs363125_C, rs3775061_A, rs3856973_G, rs4690072_T, rs4690073_G, rs6446723_T, rs6844859_T, rs762855_A, rs7659144_C, rs7685686_A, rs7691627_G, rs7694687_C, rs916171_C, and rs9993542_C. In some embodiments, an oligonucleotide comprises a base sequence complementary to rs10015979_G, rs1006798_A, rs10488840_G, rs108850_C, rs11731237_T, rs1263309_T, rs16843804_C, rs2024115_A, rs2285086_A, rs2298967_T, rs2298969_A, rs2798235_G, rs2798296_G, rs2857936_C, rs3095074_G, rs3121417_G, rs3121419_C, rs3129322_T, rs34315806_C, rs362271_G, rs362272_G, rs362273_A, rs362275_C, rs362296_C, rs362303_C, rs362306_G, rs362307_T rs362310_C, rs362331_T, rs363064_C, rs363072_A, rs363080_C, rs363088_A, rs363092_C, rs363096_T, rs363099_C, rs363125_C, rs3775061_A, rs3856973_G, rs4690072_T, rs4690073_G, rs6446723_T, rs6844859_T, rs762855_A, rs7659144_C, rs7685686_A, rs7691627_G, rs7694687_C, rs916171_C, or rs9993542_C.

In some embodiments, an HTT oligonucleotide targets an HTT site which is selected from any of the following SNPs (wherein one variant of the SNP is noted after the SNP number): rs16843804_C, rs2276881_G, rs2285086_A rs2298967_T, rs2298969_A, rs2530595_C, rs2530595_T, rs3025838_C, rs3025849_A, rs3121419_C, rs34315806_C, rs362271_G, rs362273_A, rs362303_C, rs362306_G, rs362310_C, rs362322_A, rs362331_T, rs363064_C, rs363075_G, rs363081_G, rs363088_A, rs363099_C, rs3856973_G, rs4690072_T, rs6844859_T, and rs7685686_A.

In some embodiments, an HTT oligonucleotide targets an HTT site which is selected from any of the following SNPs (wherein one variant of the SNP is noted after the SNP number): rs16843804_C, rs2276881_G, rs2285086_A rs2298967_T, rs2298969_A, rs3025838_C, rs3025849_A, rs3121419_C, rs34315806_C, rs362271_G, rs362273_A, rs362303_C, rs362306_G, rs362310_C, rs362322_A, rs362331_T rs363064_C rs363075_G rs363081_G rs363088_A, rs363099_C, rs3856973_G, rs4690072_T rs6844859_T and rs7685686_A.

In some embodiments, an HTT oligonucleotide targets an HTT site which is selected from any of the following SNPs (wherein one variant of the SNP is noted after the SNP number): rs10015979_G, rs11731237_T, rs2024115_A, rs2285086_A, rs2298969_A, rs362272_G, rs362331_T, rs363092_C, rs363096_T, rs3856973_G, rs4690072_T, rs4690073_G, rs6446723_T, rs6844859_T, rs7685686_A, rs7691627_G, and rs916171_C.

In some embodiments, an HTT oligonucleotide targets an HTT site which is selected from any of the following SNPs: rs362307, rs362331, rs1936032, rs363075, rs35892913, rs143646, rs3025837, rs362273, rs2276881, rs362272, rs363099, rs3025843, rs34315806, rs363125, rs363096, rs113407847, and rs2857790. In some embodiments, an HTT SNP has a Disease-associated allele (a variant that is more commonly in phase with the CAG expansion) and a Non-disease-associated allele (e.g., a variant which is more commonly not in phase with the CAG expansion).

| SNP | Disease-associated allele | Non-disease-associated allele |
|---|---|---|
| rs362307 | T | C |
| rs362331 | T | C |
| rs1936032 | C | G |
| rs363075 | G | A |
| rs35892913 | G | A |
| rs1143646 | T | G |
| rs3025837 | A | C |
| rs362273 | A | G |
| rs2276881 | G | A |
| rs362272 | G | A |
| rs363099 | C | T |
| rs3025843 | A | G |
| rs34315806 | C | T |
| rs363125 | C | A |
| rs363096 | T | C |
| rs2857790 | C | T |

In some embodiments, a target Huntingtin SNP site is selected from:

Frequency of Heterozygosity for 24 SNP Sites in the Huntingtin mRNA

| Location in | SNP | | |
|---|---|---|---|
| mRNA | Reference | Percent Heterozygosity | |
| (Position, nt) | Number | Controls | HD Patients |
| ORF, exon 20 | rs363075 | G/A, 10.3% (G/G, 89.7%) | G/A, 12.8% (G/G. 86.2%; A/A, |
| (2822) | | | 0.9%) |
| ORF, exon 25 | rs35892913 | G/A, 10.3% (G/G, 89.7%) | G/A, 13.0% (G/G, 86.1%; A/A, |
| (3335) | | | 0.9%) |
| ORF, exon 25 | rs1065746 | G/C, 0% (G/G, 100%) | G/C, 0.9% (G/G, 99.1%) |
| (3389) | | | |
| ORF, exon 25 | rs17781557 | T/G, 12.9% (T/T, 87.1%) | T/G, 1.9% (T/T, 98.1%) |
| (3418) | | | |
| ORF, exon 29 | rs4690074 | C/T, 37.9% (C/C, 50.9%; T/T, | C/T, 35.8% (C/C, 59.6%; T/T, |
| (3946) | | 11.2) | 4.6%) |
| ORF, exon 39 | rs363125 | C/A, 17.5% (C/C, 79.0%; A/A, | C/A, 11.0% (C/C, 87.2%; A/A, |
| (5304) | | 3.5%) | 1.8%) |
| ORF, exon 44 | exon 44 | G/A, 0% (G/G, 100%) | G/A, 2.8% (G/G, 97.2%) |
| (6150) | | | |
| ORF, exon 48 | rs362336 | G/A, 38.7% (G/G, 49.6%; A/A, | G/A, 37.4% (G/G, 57.9%; A/A, |
| (6736) | | 11.7%) | 4.7%) |
| ORF, exon 50 | rs362331 | T/C, 45.7% (T/T, 31.0%; C/C, | T/C, 39.4% (T/T, 49.5%; C/C, |
| (7070) | | 23.3%) | 11.0%) |
| ORF, exon 57 | rs362273 | A/G, 40.3% (A/A, 48.2%; G/G, | A/G, 35.2% (A/A, 60.2%; G/G, |
| (7942) | | 11.4%) | 4.6%) |
| ORF, exon 61 | rs362272 | G/A, 37.1% (G/G, 51.7%; A/A, | G/A, 36.1% (G/G, 59.3%; A/A, |
| (8501) | | 11.2%) | 4.6%) |
| ORF, exon 65 | rs3025806 | A/T, 0% (C/C, 100%) | A/T, 0% (C/C, 100%) |
| (9053) | | | |
| ORF, exon 65 | exon 65 | G/A, 2.3% (G/G, 97.7%) | G/A, 0% (G/G, 100%) |
| (9175) | | | |
| ORF, exon 67 | rs362308 | T/C, 0% (T/T, 100%) | T/C, 0% (T/T, 100%) |
| (9523) | | | |
| 3′UTR, exon 67 | rs362307 | C/T, 13.0% (C/C, 87.0%) | C/T, 48.6% (C/C, 49.5%; T/T, |
| (9633) | | | 1.9%) |
| 3′UTR, exon 67 | rs362306 | G/A, 36.0% (G/G, 52.6%; A/A, | G/A, 35.8% (G/G, 59.6%; A/A, |
| (9888) | | 11.4%) | 4.6%) |
| 3′UTR, exon 67 | rs362268 | C/G, 36.8% (C/C, 50.0%; G/G | C/G, 35.8% (C/C, 59.6%; G/G, |
| (9936) | | 13.2%) | 4.6%) |
| 3′UTR, exon 67 | rs362305 | C/G, 20.2% (C/C, 78.1%; G/G | C/G, 11.9% (C/C, 85.3%; G/G, |
| (9948) | | 1.8%) | 2.8%) |
| 3′UTR, exon 67 | rs362304 | C/A, 22.8% (C/C, 73.7%; A/A, | C/A, 11.9% (C/C, 85.3%; AA, |
| (10060) | | 3.5%) | 2.8%) |
| 3′UTR, exon 67 | rs362303 | C/T, 18.4% (C/C, 79.8%; T/T, | C/A, 11.9% (C/C, 85.3%; T/T, |
| (10095) | | 1.8%) | 2.8%) |
| 3′UTR, exon 67 | rs1557210 | C/T, 0% (C/C, 100%) | C/T, 0% (C/C, 100%) |
| (10704) | | | |
| 3′UTR, exon 67 | rs362302 | C/T, 4.3% (C/C, 95.7%) | C/T, 0% (C/C, 100%) |
| (10708) | | | |
| 3′UTR, exon 67 | rs3025805 | G/T, 0% (G/G, 100%) | G/T, 0% (G/G, 100%) |
| (10796) | | | |
| 3′UTR, exon 67 | rs362267 | C/T, 36.2% (C/C, 52.6%; T/T, | C/T, 35.5% (C/C, 59.8%; T/T, |
| (11006) | | 11.2%) | 4.7%) |

At least one of SNPs has been reported as being difficult to target with an oligonucleotide to reduce expression, level and/or activity of HTT or a product thereof, especially with selectivity for mutant HTT. Among other things, the present disclosure provides technologies, e.g., oligonucleotides, compositions, methods, etc., for targeting such difficult SNPs (and others) to reduce expression, level and/or activity of HTT or a product thereof, in many cases, selectively of mutant HTT or a product thereof.

In some embodiments, a targeted HTT SNP is rs362268.

In some embodiments, a muHTT transcript, e.g., mRNA, comprising SNP rs362268 comprises a sequence (5′-3′) of UGC AGG CUG GCU GUU GGC CC (wherein the SNP is in bold, underlined text), and wherein the corresponding portion of the wild-type allele has the sequence UGC AGG CUG GGU GUU GGC CC, and wherein an HTT oligonucleotide targeting the SNP has a base sequence comprising the sequence of GGGCCAACAGCCAGCCTGCA (wherein the base capable of basepairing with the SNP is in bold, underlined text) or a span of the sequence which is at least 8 bases long and comprises the base capable of basepairing with the SNP.

In some embodiments, an HTT oligonucleotide targets HTT SNP rs362268 and has a base sequence comprising the SNP (or the complement of a base sequence comprising the SNP) or has a base sequence comprising a wild-type base corresponding to the SNP (or the complement thereof). In some embodiments, an HTT oligonucleotide targets HTT SNP rs362268 and is: WV-949, WV-960, WV-961, WV-962, WV-963, WV-964, WV-965, WV-1031, WV-1032, WV-1033, WV-1034, WV-1035, WV-1036, WV-1037, WV-1038, WV-1039, WV-1040, WV-1041, WV-1042, WV-1043, WV-1044, WV-1045, WV-1046, WV-1047, WV-1048, WV-1049, WV-1050, WV-1051, WV-1052, WV-1053, WV-1054, WV-1055, WV-1056, WV-1057, WV-1058, WV-1059, or WV-1060. In some embodiments, a base sequence of an oligonucleotide comprises at least 10 contiguous bases of any of these oligonucleotides and which comprises the SNP. Sequences, data and other information related to various HTT oligonucleotides to this SNP are presented herein and in WO2017015555 and WO2017/192664.

Non-limiting examples of HTT oligonucleotides which target rs362268 include the following: WV-1031, WV-1032, WV-1033, WV-1034, WV-1035, WV-1036, WV-1037, WV-1038, WV-1039, WV-1040, WV-1041, WV-1042, WV-1043, WV-1044, WV-1045, WV-1046, WV-1047, WV-1048, WV-1049, WV-1050, WV-1051, WV-1052, WV-1053, WV-1054, WV-1055, WV-1056, WV-1057, WV-1058, WV-1059, WV-1060, WV-960, WV-961, WV-962, WV-963, WV-964, and WV-965. In some embodiments, abase sequence of an oligonucleotide comprises at least 10 contiguous bases of any of these oligonucleotides and which comprises the SNP.

An oligonucleotide having the sequence of a mRNA fragment comprising the wild-type isoform of this SNP is WV-958; an oligonucleotide having the sequence of a mRNA fragment comprising the mutant isoform of this SNP is WV-959.

In some embodiments, a base sequence of an oligonucleotide is, comprises, or comprises at least 10 contiguous bases of, GGGCCAACAGCCAGCCTGCA, wherein each U may be independently replaced with T, and/or each T may be independently replaced with U. In some embodiments, a base sequence of an oligonucleotide is, comprises, or comprises at least 10 contiguous bases of, GGGCCAACACCCAGCCTGCA, wherein each U may be independently replaced with T, and/or each T may be independently replaced with U.

In some embodiments, a targeted HTT SNP is rs362272.

In some embodiments, an HTT oligonucleotide targets HTT SNP rs362272 and has a base sequence comprising the SNP (or the complement of a base sequence comprising the SNP) or has a base sequence comprising a wild-type base corresponding to the SNP (or the complement thereof). In some embodiments, an HTT oligonucleotide targets HTT SNP rs362272 and is: WV-10989, WV-10990, WV-10991, WV-10992, WV-10993, WV-10994, WV-10995, WV-10996, WV-10997, WV-10998, WV-10999, WV-11000, WV-11001, WV-11002, WV-11003, WV-11004, WV-11005, WV-11006, WV-11007, WV-11008, WV-11009, WV-11010, WV-11011, WV-11012, WV-11013, WV-11014, WV-11015, WV-11016, WV-11017, WV-11018, WV-11019, WV-11020, WV-11021, WV-11022, WV-11023, WV-11024, WV-11025, WV-11026, WV-11027, WV-11028, WV-11029, WV-11030, WV-11031, WV-11032, WV-11033, WV-11034, WV-11035, WV-11036, WV-11037, WV-11038, WV-13411, WV-13412, WV-13413, WV-13414, WV-13415, WV-13416, WV-13417, WV-13418, WV-13419, WV-13420, WV-13421, WV-13422, WV-13423, WV-13424, WV-13425, WV-13426, WV-13427, WV-13428, WV-13429, WV-13430, WV-13431, WV-13432, WV-13433, WV-13434, WV-13435, WV-13436, WV-13437, or WV-13438. In some embodiments, abase sequence of an oligonucleotide comprises at least 10 contiguous bases of any of these oligonucleotides and which comprises the SNP.

In some embodiments, a targeted HTT SNP is rs362273.

In some embodiments, an HTT oligonucleotide targets HTT SNP rs362273 and has a base sequence comprising the SNP (or the complement of a base sequence comprising the SNP) or has a base sequence comprising a wild-type base corresponding to the SNP (or the complement thereof). In some embodiments, an HTT oligonucleotide targets HTT SNP rs362273 and is: WV-10939, WV-10940, WV-10941, WV-10942, WV-10943, WV-10944, WV-10945, WV-10946, WV-10947, WV-10948, WV-10949, WV-10950, WV-10951, WV-10952, WV-10953, WV-10954, WV-10955, WV-10956, WV-10957, WV-10958, WV-10959, WV-10960, WV-10961, WV-10962, WV-10963, WV-10964, WV-10965, WV-10966, WV-10967, WV-10968, WV-10969, WV-10970, WV-10971, WV-10972, WV-10973, WV-10974, WV-10975, WV-10976, WV-10977, WV-10978, WV-10979, WV-10980, WV-10981, WV-10982, WV-10983, WV-10984, WV-10985, WV-10986, WV-10987, WV-10988, WV-12258, WV-12259, WV-12260, WV-12261, WV-12262, WV-12263, WV-12264, WV-12265, WV-12266, WV-12267, WV-12268, WV-12269, WV-12270, WV-12271, WV-12272, WV-12273, WV-12274, WV-12275, WV-12276, WV-12277, WV-12278, WV-12279, WV-12280, WV-12281, WV-12282, WV-12283, WV-12284, WV-12285, WV-12286, WV-12287, WV-12425, WV-12426, WV-12427, WV-12428, WV-12429, WV-12430, WV-12431, WV-12432, WV-12433, WV-12434, WV-12435, WV-12436, WV-12437, WV-12438, WV-14059, WV-14060, WV-14061, WV-14062, WV-14063, WV-14064, WV-14065, WV-14066, WV-14067, WV-14068, WV-14069, WV-14070, WV-14071, WV-14072, WV-14073, WV-14074, WV-14075, WV-14076, WV-14077, WV-14078, WV-14079, WV-14080, WV-14081, WV-14082, WV-14083, WV-14084, WV-14085, WV-14086, WV-14092, WV-14093, WV-14094, WV-14095, WV-14096, WV-14097, WV-14098, WV-14099, WV-14100, WV-14101, WV-14712, WV-14713, WV-14759, WV-14914, WV-14915, WV-15077, WV-15078, WV-15079, WV-15080, WV-16214, WV-16215, WV-16216, WV-16217, WV-16218, WV-17776, WV-17777, WV-17778, WV-17779, WV-17780, WV-17781, WV-17782, WV-17783, WV-17784, WV-17785, WV-17786, WV-17787, WV-17788, WV-17789, WV-17790, WV-17791, WV-17792, WV-17793, WV-17794, WV-17795, WV-17796, WV-17797, WV-17798, WV-17799, WV-17800, WV-19819, WV-19820, WV-19821, WV-19822, WV-19823, WV-19824, WV-19825, WV-19826, WV-19827, WV-19828, WV-19829, WV-19830, WV-19831, WV-19832, WV-19833, WV-19834, WV-19835, WV-19836, WV-19837, WV-19838, WV-19839, WV-19840, WV-19841, WV-19842, WV-19843, WV-19844, WV-19845, WV-19846, WV-19847, WV-19848, WV-19849, WV-19850, WV-19851, WV-19852, WV-19853, WV-19854, or WV-19855. In some embodiments, a base sequence of an oligonucleotide comprises at least 10 contiguous bases of any of these oligonucleotides and which comprises the SNP.

In some embodiments, a targeted HTT SNP is rs362306.

In some embodiments, a muHTT transcript, e.g., mRNA, comprising SNP rs362306 comprises a sequence (5′-3′) of UUG CCA GGU UGC AGC UGC UC (wherein the SNP is in bold, underlined text), and wherein the corresponding portion of the wild-type allele has the sequence UUG CCA GGU UAC AGC UGC UC, and wherein an HTT oligonucleotide targeting the SNP has a base sequence comprising the sequence of GAGCAGCTGCAACCTGGCAA (wherein the base capable of basepairing with the SNP is in bold, underlined text) or a span of the sequence which is at least 8 bases long and comprises the base capable of basepairing with the SNP.

In some embodiments, an HTT oligonucleotide, e.g., which targets a mutant (mu) allele of this SNP, is WV-951, or any oligonucleotide which comprises at least 10 contiguous base of the base sequence of this HTT oligonucleotide and which comprises the SNP. In some embodiments, an HTT oligonucleotide, e.g., which targets a wt (wild-type) allele of this SNP, is WV-950, or any oligonucleotide which comprises at least 10 contiguous base of the base sequence of this HTT oligonucleotide and which comprises the SNP.

In some embodiments, an HTT oligonucleotide targets HTT SNP rs362306 and has a base sequence comprising the SNP (or the complement of a base sequence comprising the SNP) or has a base sequence comprising a wild-type base corresponding to the SNP (or the complement thereof).

Non-limiting examples of HTT oligonucleotides which target rs362306 include the following: WV-1001, WV-1002, WV-1003, WV-1004, WV-1005, WV-1006, WV-1007, WV-1008, WV-1009, WV-1010, WV-1011, WV-1012, WV-1013, WV-1014, WV-1015, WV-1016, WV-1017, WV-1018, WV-1019, WV-1020, WV-1021, WV-1022, WV-1023, WV-1024, WV-1025, WV-1026, WV-1027, WV-1028, WV-1029, WV-1030, WV-952, WV-953, WV-954, WV-955, WV-956, and WV-957. In some embodiments, abase sequence of an oligonucleotide comprises at least 10 contiguous bases of any of these oligonucleotides and which comprises the SNP.

In some embodiments, an HTT oligonucleotide targets HTT SNP rs362306 and has a base sequence comprising the SNP (or the complement of a base sequence comprising the SNP) or has a base sequence comprising a wild-type base corresponding to the SNP (or the complement thereof). In some embodiments, an HTT oligonucleotide targets HTT SNP rs362306 and is: WV-948, WV-950, WV-951, WV-952, WV-953, WV-954, WV-955, WV-956, WV-957, WV-1001, WV-1002, WV-1003, WV-1004, WV-1005, WV-1006, WV-1007, WV-1008, WV-1009, WV-1010, WV-1011, WV-1012, WV-1013, WV-1014, WV-1015, WV-1016, WV-1017, WV-1018, WV-1019, WV-1020, WV-1021, WV-1022, WV-1023, WV-1024, WV-1025, WV-1026, WV-1027, WV-1028, WV-1029, or WV-1030. In some embodiments, a base sequence of an oligonucleotide comprises at least 10 contiguous bases of any of these oligonucleotides and which comprises the SNP.

Sequences, data and other information related to various HTT oligonucleotides to this SNP are presented herein and in WO2017015555 and WO2017192664.

In some embodiments, a targeted HTT SNP is rs362307.

In some embodiments, a muHTT transcript, e.g., mRNA, comprising SNP rs362307 comprises a sequence (5′-3′) of UGG AAG UCU GUG CCC UUG UG (wherein the SNP is in bold, underlined text, and the wild-type base at this position is C), and wherein the corresponding portion of the wild-type allele has the sequence UGG AAG UCU GCG CCC UUG UG, and wherein an HTT oligonucleotide targeting the SNP has a base sequence comprising the sequence of CACAAGGGCACAGACTTCCA (wherein the base capable of basepairing with the SNP is in bold, underlined text) or a span of the sequence which is at least 8 bases long and comprises the base capable of basepairing with the SNP. The U isoform of SNP rs362307 at Huntingtin mRNA nucleotide 9,633 is often associated with (e.g., in phase with) the expanded CAG Disease Allele.

In some embodiments, an HTT oligonucleotide targets HTT SNP_rs362307 and has a base sequence comprising the SNP (or the complement of a base sequence comprising the SNP) or has a base sequence comprising a wild-type base corresponding to the SNP (or the complement thereof).

Non-limiting examples of HTT oligonucleotides which target rs362307 include the following: WV-904, WV-905, WV-906, WV-907, WV-908, WV-909, WV-910, WV-911, WV-912, WV-913, WV-914, WV-915, WV-916, WV-917, WV-918, WV-919, WV-920, WV-921, WV-922, WV-923, WV-924, WV-925, WV-926, WV-927, WV-928, WV-929, WV-930, WV-931, WV-932, WV-933, WV-934, WV-935, WV-936, WV-937, WV-938, WV-939, WV-940, WV-941, WV-1085, WV-1086, WV-1087, WV-1088, WV-1089, WV-1090, WV-1091, WV-1092, WV-982, WV-983, WV-984, WV-985, WV-986, WV-987, WV-1234, WV-1235, WV-1067, WV-1068, WV-1069, WV-1070, WV-1071, WV-1072, WV-1510, WV-1511, WV-1497, and WV-1655. In some embodiments, a base sequence of an oligonucleotide comprises at least 10 contiguous bases of any of these oligonucleotides and which comprises the SNP.

In some embodiments, an HTT oligonucleotide targets HTT SNP_rs362307 and is: WV-905, WV-906, WV-907, WV-908, WV-909, WV-911, WV-912, WV-913, WV-914, WV-915, WV-921, WV-935, WV-937, WV-938, WV-939, WV-940, WV-941, WV-985, WV-986, WV-987, WV-1068, WV-1069, WV-1071, WV-1072, WV-1088, WV-1089, WV-1090, WV-1198, WV-1199, WV-1200, WV-1201, WV-1202, WV-1203, WV-1204, WV-1205, WV-1206, WV-1207, WV-1208, WV-1209, WV-1210, WV-1211, WV-1212, WV-1213, WV-1214, WV-1215, WV-1216, WV-1235, WV-1654, WV-1655, WV-2623, WV-13646, WV-13647, WV-13648, WV-13649, WV-13650, WV-13651, WV-13652, WV-13653, WV-13654, WV-13655, WV-13656, WV-13657, WV-13658, WV-13659, WV-13660, WV-13661, WV-13662, WV-13663, WV-13664, WV-13665, WV-13666, WV-13935, WV-13936, WV-13940, WV-13941, WV-13942, WV-13943, WV-13944, WV-13945, WV-13946, WV-13947, WV-13948, WV-13949, WV-13957, WV-13958, WV-13961, WV-13962, WV-15634, WV-15635, WV-15636, WV-15637, WV-17895, WV-17896, WV-17897, WV-17898, WV-904, WV-905, WV-906, WV-907, WV-908, WV-909, WV-910, WV-911, WV-912, WV-913, WV-914, WV-915, WV-916, WV-917, WV-918, WV-919, WV-920, WV-921, WV-922, WV-923, WV-924, WV-925, WV-926, WV-927, WV-928, WV-929, WV-930, WV-931, WV-932, WV-933, WV-934, WV-935, WV-936, WV-937, WV-938, WV-939, WV-940, WV-941, WV-982, WV-983, WV-984, WV-985, WV-1067, WV-1068, WV-1069, WV-1070, WV-1071, WV-1072, WV-1085, WV-1086, WV-1087, WV-1088, WV-1089, WV-1090, WV-1091, WV-1092, WV-1183, WV-1184, WV-1185, WV-1186, WV-1187, WV-1188, WV-1189, WV-1190, WV-1191, WV-1192, WV-1193, WV-1194, WV-1195, WV-1196, WV-1197, WV-1198, WV-1199, WV-1200, WV-1201, WV-1202, WV-1203, WV-1204, WV-1234, WV-1235, WV-1497, WV-1510, WV-1511, WV-1654, WV-1655, WV-1788, WV-2022, WV-2377, WV-2378, WV-2379, WV-2380, WV-2623, WV-2659, WV-2676, WV-2682, WV-2683, WV-2684, WV-2685, WV-2686, WV-2687, WV-2688, WV-2689, WV-2690, WV-2691, WV-2692, WV-2732, WV-4241, WV-4242, WV-4278, WV-5141, WV-5142, WV-5143, WV-5144, WV-5145, WV-5146, WV-5147, WV-5148, WV-5149, WV-5150, WV-5151, WV-5152, WV-5159, WV-5160, WV-5161, WV-5162, WV-5163, WV-5164, WV-5165, WV-5166, WV-5167, WV-5168, WV-5169, WV-5170, WV-5177, WV-5178, WV-5179, WV-5180, WV-5181, WV-5182, WV-5183, WV-5184, WV-5185, WV-5186, WV-5187, WV-5188, WV-5189, WV-5190, WV-5197, WV-5198, WV-5199, WV-5200, WV-5201, WV-5202, WV-5203, WV-5204, WV-5205, WV-5206, WV-5207, WV-5208, WV-5209, WV-5210, WV-6013, WV-6014, WV-6506, WV-8706, WV-8707, WV-8708, WV-8709, WV-9854, WV-9855, WV-10113, WV-10114, WV-10115, WV-10116, WV-10117, WV-10118, WV-10119, WV-10120, WV-10121, WV-10122, WV-10123, WV-10124, WV-10125, WV-10126, WV-10133, WV-10134, WV-10135, WV-10136, WV-10137, WV-10138, WV-10139, WV-10140, WV-10141, WV-10142, WV-10143, WV-10144, WV-10145, WV-10146, WV-10483, WV-10484, WV-10485, WV-10486, WV-10640, WV-10641, WV-13646, WV-13647, WV-13648, WV-13649, WV-13650, WV-13651, WV-13652, WV-13653, WV-13654, WV-13655, WV-13656, WV-13657, WV-13658, WV-13659, WV-13660, WV-13661, WV-13662, WV-13663, WV-13664, WV-13665, WV-13666, WV-13935, WV-13936, WV-13937, WV-13938, WV-13939, WV-13940, WV-13941, WV-13942, WV-13943, WV-13944, WV-13945, WV-13946, WV-13947, WV-13948, WV-13949, WV-13953, WV-13954, WV-13957, WV-13958, WV-13961, WV-13962, WV-14133, WV-14134, WV-14135, WV-14136, WV-15634, WV-15635, WV-15636, WV-15637, WV-15642, WV-15643, WV-15644, WV-15645, WV-17895, WV-17896, WV-17897, WV-17898, WV-17899, WV-17900, WV-17901, WV-17902, WV-17903, WV-17904, WV-17905, WV-17906, WV-17907, WV-17908, WV-17909, WV-17910, WV-17911, WV-17912, WV-17913, WV-17914, WV-17915, WV-17916, WV-17917, WV-17918, WV-19872, WV-19873, WV-19874, WV-19875, WV-19876, WV-19877, WV-19878, WV-19879, WV-19880, WV-19881, WV-19882, or WV-19883. In some embodiments, a base sequence of an oligonucleotide comprises at least 10 contiguous bases of any of these oligonucleotides and which comprises the SNP. Sequences, data and other information related to various HTT oligonucleotides to this SNP are presented herein and in WO2017015555 and WO2017192664.

In some embodiments, an HTT oligonucleotide has a sequence which comprises the wild-type base at the position corresponding to SNP rs362307. Non-limiting examples of such an oligonucleotide include: WV-9660, WV-9661, WV-9662, WV-9663, WV-9664, WV-9665, WV-9666, WV-9667, WV-9668, WV-9669, WV-9692, WV-9693, WV-10767, WV-10768, WV-10769, WV-10770, WV-10771, WV-10772, WV-10773, WV-10774, WV-10775, WV-10776, WV-10862, WV-10863, WV-11534, WV-11535, WV-11536, WV-11537, WV-11538, WV-11539, WV-11540, WV-11541, WV-11542, WV-11543, WV-11968, WV-11969, WV-11970, WV-11971, WV-11972, WV-11973, WV-11974, WV-11975, WV-11976, WV-11977, WV-11978, WV-11979, WV-11980, WV-11981, WV-11982, WV-11983, WV-11984, WV-11985, WV-11986, WV-11987, WV-11988, WV-11989, WV-11990, WV-11991, WV-11992, WV-11993, WV-11994, WV-11995, WV-11996, WV-11997, WV-11998, WV-11999, WV-12000, WV-12001, WV-12002, WV-12003, WV-12004, WV-12005, WV-12006, WV-12007, WV-12013, WV-12014, WV-12015, WV-12016, WV-12017, WV-12018, WV-12019, WV-12020, WV-12021, WV-12022, WV-12033, WV-12034, WV-12035, WV-12036, WV-12037, WV-12038, WV-12039, WV-12040, WV-12041, WV-12042, WV-12288, WV-12289, WV-12290, WV-12291, WV-12292, WV-12293, WV-12294, WV-12295, WV-12296, WV-12297, WV-12298, WV-12299, WV-12300, WV-12301, WV-12302, WV-12544, WV-13625, WV-13626, WV-13627, WV-13628, WV-13629, WV-13630, WV-13631, WV-13632, WV-13633, WV-13634, WV-13635, WV-13636, WV-13637, WV-13638, WV-13639, WV-13640, WV-13641, WV-13642, WV-13643, WV-13644, WV-13645, WV-13667, WV-13920, WV-13921, WV-13922, WV-13923, WV-13924, WV-13925, WV-13926, WV-13927, WV-13928, WV-13929, WV-13930, WV-13932, WV-13933, WV-13934, WV-13950, WV-13951, WV-13952, WV-13955, WV-13956, WV-13959, WV-13960, WV-15630, WV-15631, WV-15632, WV-15633, WV-15638, WV-15639, WV-15640, WV-15641, WV-17886, WV-17887, WV-17888, WV-17889, WV-17890, WV-17891, WV-17892, WV-17893, WV-17894, WV-11970, WV-11971, WV-11972, WV-11973, WV-11974, WV-11975, and WV-11976. In some embodiments, a base sequence of an oligonucleotide comprises at least 10 contiguous bases of any of these oligonucleotides and which comprises the SNP.

In some embodiments, an oligonucleotide, e.g., an HTT oligonucleotide, comprising the wt isoform of a SNP is useful for testing in cells and/or animals which are wild-type in both alleles at that SNP. In some embodiments, an oligonucleotide, e.g., an HTT oligonucleotide, comprising the wt isoform of a SNP can be used in such wild-type cells and/or animals as a surrogate of an oligonucleotide, e.g., an HTT oligonucleotide, comprising the mutant isoform of the SNP. Non-limiting examples of a wt surrogate of a mutant HTT oligonucleotide include: WV-9660, WV-9661, WV-9662, WV-9663, WV-9664, WV-9665, WV-9666, WV-9667, WV-9668, WV-9669, WV-9692, and WV-9693.

In some embodiments, a targeted HTT SNP is rs362331.

In some embodiments, an HTT oligonucleotide targets HTT SNP_rs362331 and has a base sequence comprising the SNP (or the complement of a base sequence comprising the SNP) or has a base sequence comprising a wild-type base corresponding to the SNP (or the complement thereof). In some embodiments, an HTT oligonucleotide targets HTT SNP rs362331 and is: WV-2597, WV-2598, WV-2599, WV-2600, WV-2601, WV-2602, WV-2603, WV-2604, WV-2613, WV-2614, WV-2615, WV-2616, WV-2617, WV-2618, WV-2619, WV-2620, WV-2642, WV-2643, WV-3857, WV-4279, WV-5211, WV-5212, WV-5213, WV-5214, WV-5215, WV-5216, WV-5217, WV-5218, WV-5219, WV-5220, WV-5221, WV-5222, WV-5223, WV-5224, WV-5225, WV-5226, WV-5227, WV-5228, WV-5229, WV-5230, WV-5231, WV-5232, WV-5233, WV-5234, WV-5235, WV-5236, WV-5237, WV-5238, WV-5239, WV-5240, WV-5241, WV-5242, WV-5243, WV-5244, WV-5245, WV-5246, WV-5247, WV-5248, WV-5249, WV-5250, WV-5251, WV-5252, WV-5253, WV-5254, WV-5255, WV-5256, WV-5257, WV-5258, WV-5259, WV-5260, WV-5261, WV-5262, WV-5263, WV-5264, WV-5265, WV-5266, WV-5267, WV-5268, WV-5269, WV-5270, WV-5271, WV-5272, WV-5273, WV-5274, WV-5275, WV-5276, WV-5277, WV-5278, WV-5279, WV-5280, WV-5281, WV-5282, WV-5283, WV-5284, WV-5285, WV-5286, WV-8710, WV-8711, WV-8712, WV-8713, WV-9856, WV-9857, WV-10631, WV-10632, WV-10633, WV-10642, WV-10643, WV-10644, WV-10864, WV-10865, WV-10866, WV-10867, WV-11115, WV-11116, WV-11117, WV-11118, WV-11119, WV-11120, WV-11121, WV-11122, WV-11123, WV-11124, WV-11125, WV-11126, WV-11127, WV-11128, WV-11129, WV-11130, WV-11131, WV-11132, WV-11548, WV-11549, WV-11550, WV-11551, WV-11552, WV-11553, WV-11554, WV-11555, WV-11556, WV-11557, WV-11558, WV-11559, WV-11560, WV-11561, WV-11562, WV-11563, WV-11564, WV-11565, WV-11566, WV-11567, WV-12049, WV-12539, WV-12540, WV-12541, WV-12542, WV-12543, WV-15133, WV-15134, WV-15135, WV-15136, WV-15137, WV-15138, WV-15139, WV-15140, WV-15141, or WV-15142. In some embodiments, a base sequence of an oligonucleotide comprises at least 10 contiguous bases of any of these oligonucleotides and which comprises the SNP. Sequences, data and other information related to various HTT oligonucleotides to this SNP are presented herein and in WO2017015555 and WO2017192664.

In some embodiments, a targeted HTT SNP is rs363099.

In some embodiments, an HTT oligonucleotide targets HTT SNP_rs363099 and has a base sequence comprising the SNP (or the complement of a base sequence comprising the SNP) or has a base sequence comprising a wild-type base corresponding to the SNP (or the complement thereof). In some embodiments, an HTT oligonucleotide targets HTT SNP_rs363099 and is: WV-10889, WV-10890, WV-10891, WV-10892, WV-10893, WV-10894, WV-10895, WV-10896, WV-10897, WV-10898, WV-10899, WV-10900, WV-10901, WV-10902, WV-10903, WV-10904, WV-10905, WV-10906, WV-10907, WV-10908, WV-10909, WV-10910, WV-10911, WV-10912, WV-10913, WV-10914, WV-10915, WV-10916, WV-10917, WV-10918, WV-10919, WV-10920, WV-10921, WV-10922, WV-10923, WV-10924, WV-10925, WV-10926, WV-10927, WV-10928, WV-10929, WV-10930, WV-10931, WV-10932, WV-10933, WV-10934, WV-10935, WV-10936, WV-10937, WV-10938, WV-12509, WV-12510, WV-12511, WV-12512, WV-12513, WV-12514, WV-12515, WV-12516, WV-12517, WV-12518, WV-12519, WV-12520, WV-12521, WV-12522, WV-12523, WV-12524, WV-12525, WV-12526, WV-12527, WV-12528, WV-12529, WV-12530, WV-12531, WV-12532, WV-12533, WV-12534, WV-12535, WV-12536, WV-12537, or WV-12538. In some embodiments, a base sequence of an oligonucleotide comprises at least 10 contiguous bases of any of these oligonucleotides and which comprises the SNP.

In some embodiments, a targeted HTT SNP is rs2530595.

In some embodiments, an HTT oligonucleotide targets HTT SNP rs2530595 and has abase sequence comprising the SNP (or the complement of a base sequence comprising the SNP) or has a base sequence comprising a wild-type base corresponding to the SNP (or the complement thereof). In some embodiments, an HTT oligonucleotide targets HTT SNP rs2530595 and is: WV-2589, WV-2590, WV-2591, WV-2592, WV-2593, WV-2594, WV-2595, WV-2596, WV-2605, WV-2606, WV-2607, WV-2608, WV-2609, WV-2610, WV-2611, WV-2612, WV-2671, WV-2672, WV-2673, or WV-2674. In some embodiments, abase sequence of an oligonucleotide comprises at least 10 contiguous bases of any of these oligonucleotides and which comprises the SNP. Sequences, data and other information related to various HTT oligonucleotides to this SNP are presented herein and in WO2017015555 and WO2017192664.

In some embodiments, a targeted HTT SNP is rs2830088.

In some embodiments, an HTT oligonucleotide targets HTT SNP rs2830088 and has abase sequence comprising the SNP (or the complement of a base sequence comprising the SNP) or has a base sequence comprising a wild-type base corresponding to the SNP (or the complement thereof). In some embodiments, an HTT oligonucleotide targets HTT SNP rs2830088 and is: WV-15157, WV-15158, WV-15159, WV-15160, WV-15161, WV-15175, WV-15176, WV-15177, WV-15178, WV-15179, WV-15193, WV-15194, WV-15195, WV-15196, WV-15197, WV-15211, WV-15212, WV-15213, WV-15214, or WV-15215. In some embodiments, a base sequence of an oligonucleotide comprises at least 10 contiguous bases of any of these oligonucleotides and which comprises the SNP.

In some embodiments, a targeted HTT SNP is rs7685686.

Non-limiting examples of HTT oligonucleotides which target rs7685686 include the following: ONT-450, ONT-451, ONT-452, WV-1077, WV-1078, WV-1079, WV-1080, WV-1081, WV-1082, WV-1083, WV-1084, WV-1508, WV-1509, WV-2023, WV-2024, WV-2025, WV-2026, WV-2027, WV-2028, WV-2029, WV-2030, WV-2031, WV-2032, WV-2033, WV-2034, WV-2035, WV-2036, WV-2037, WV-2038, WV-2039, WV-2040, WV-2041, WV-2042, WV-2043, WV-2044, WV-2045, WV-2046, WV-2047, WV-2048, WV-2049, WV-2050, WV-2051, WV-2052, WV-2053, WV-2054, WV-2055, WV-2056, WV-2057, WV-2058, WV-2059, WV-2060, WV-2061, WV-2062, WV-2063, WV-2064, WV-2065, WV-2066, WV-2067, WV-2068, WV-2069, WV-2070, WV-2071, WV-2072, WV-2073, WV-2074, WV-2075, WV-2076, WV-2077, WV-2078, WV-2079, WV-2080, WV-2081, WV-2082, WV-2083, WV-2084, WV-2085, WV-2086, WV-2087, WV-2088, WV-2089, and WV-2090. In some embodiments, a base sequence of an oligonucleotide comprises at least 10 contiguous bases of any of these oligonucleotides and which comprises the SNP.

In some embodiments, an HTT oligonucleotide targets HTT SNP rs7685686 and has abase sequence comprising the SNP (or the complement of a base sequence comprising the SNP) or has a base sequence comprising a wild-type base corresponding to the SNP (or the complement thereof). In some embodiments, an HTT oligonucleotide targets HTT SNP rs7685686 and is selected from any of: WV-1077, WV-1078, WV-1079, WV-1080, WV-1081, WV-1082, WV-1083, WV-1084, WV-1508, WV-1509, WV-2023, WV-2024, WV-2025, WV-2026, WV-2027, WV-2028, WV-2029, WV-2030, WV-2031, WV-2032, WV-2033, WV-2034, WV-2035, WV-2036, WV-2037, WV-2038, WV-2039, WV-2040, WV-2041, WV-2042, WV-2043, WV-2044, WV-2045, WV-2046, WV-2047, WV-2048, WV-2049, WV-2050, WV-2051, WV-2052, WV-2053, WV-2054, WV-2055, WV-2056, WV-2057, WV-2058, WV-2059, WV-2060, WV-2061, WV-2062, WV-2063, WV-2064, WV-2065, WV-2066, WV-2067, WV-2068, WV-2069, WV-2070, WV-2071, WV-2072, WV-2073, WV-2074, WV-2075, WV-2076, WV-2077, WV-2078, WV-2079, WV-2080, WV-2081, WV-2082, WV-2083, WV-2084, WV-2085, WV-2086, WV-2087, WV-2088, WV-2089, WV-2090, WV-2163, WV-2164, WV-2269, WV-2270, WV-2271, WV-2272, WV-2374, WV-2375, WV-2416, WV-2417, WV-2418, and WV-2419. In some embodiments, an oligonucleotide has a base sequence which comprises at least 10 contiguous bases of any of these oligonucleotides (or the wild-type equivalent, which comprises the wild-type nucleotide at the SNP position) or a complement thereof and which comprises the SNP. Sequences, data and other information related to various HTT oligonucleotides to this SNP are presented herein and in WO2017015555 and WO2017192664.

In some embodiments, a targeted HTT SNP is intronic.

In some embodiments, an HTT oligonucleotide targets a SNP which is intronic.

In some embodiments, an HTT oligonucleotide targets an intronic HTT SNP and has a base sequence comprising the SNP (or the complement of a base sequence comprising the SNP) or has a base sequence comprising a wild-type base corresponding to the SNP (or the complement thereof).

Non-limiting examples of such oligonucleotides include: WV-10783, WV-10784, WV-10785, WV-10786, WV-10787, WV-10788, WV-10789, WV-10790, WV-10791, WV-10792, WV-10793, WV-10794, WV-10795, WV-10796, WV-10797, WV-10798, WV-10799, WV-10800, WV-10801, WV-10802, WV-10803, WV-10804, WV-10805, WV-10806, WV-10807, WV-10808, WV-10809, WV-10810, WV-10811, WV-10812, WV-10813, WV-10814, WV-10815, WV-10816, and WV-10817.

In some embodiments, a base basepairing to a base at a SNP site (a SNP base; a base basepairing to a SNP base a SNP-pairing base) in a transcript, e.g., an HTT mRNA, can be located at various position of an oligonucleotide, e.g., an HTT oligonucleotide. In some embodiments, a SNP-pairing base is located at position 1, 2, 3, 4, 5, 6, 7, 8, 9, 19, 11, 12, 13, 14, 15, 16, 17, 18, 19, 20, 21, 22, 23, 24, 25, 26, 27, 28, 29 or 30 (counting from the 5′ end) of an oligonucleotide. In some embodiments, the position 1 (counting from the 5′ end) is also designated P1; the position 2 (counting from the 5′ end) is also designated P2; etc. In some embodiments, an oligonucleotide, e.g., an HTT oligonucleotide, comprises a SNP-pairing base at position 1, 2, 3, 4, 5, 6, 7, 8, 9, 19, 11, 12, 13, 14, 15, 16, 17, 18, 19, 20, 21, 22, 23, 24, 25, 26, 27, 28, 29 or 30 (counting from the 5′ end).

In some embodiments, an oligonucleotide, e.g., an HTT oligonucleotide, comprises a SNP-pairing base at Position P1 (of the oligonucleotide, wherein the position is counted as a number of bases from 5′ to 3′). In some embodiments, an oligonucleotide, e.g., an HTT oligonucleotide, comprises a SNP-pairing base at Position P2. In some embodiments, an oligonucleotide, e.g., an HTT oligonucleotide, comprises a SNP-pairing base at Position P3. In some embodiments, an oligonucleotide, e.g., an HTT oligonucleotide, comprises a SNP-pairing base at Position P4. In some embodiments, an oligonucleotide, e.g., an HTT oligonucleotide, comprises a SNP-pairing base at Position P5. In some embodiments, an oligonucleotide, e.g., an HTT oligonucleotide, comprises a SNP-pairing base at Position P6. In some embodiments, an oligonucleotide, e.g., an HTT oligonucleotide, comprises a SNP-pairing base at Position P7. In some embodiments, an oligonucleotide, e.g., an HTT oligonucleotide, comprises a SNP-pairing base at Position P8. In some embodiments, an oligonucleotide, e.g., an HTT oligonucleotide, comprises a SNP-pairing base at Position P9. In some embodiments, an oligonucleotide, e.g., an HTT oligonucleotide, comprises a SNP-pairing base at Position P10. In some embodiments, an oligonucleotide, e.g., an HTT oligonucleotide, comprises a SNP-pairing base at Position P11. In some embodiments, an oligonucleotide, e.g., an HTT oligonucleotide, comprises a SNP-pairing base at Position P12. In some embodiments, an oligonucleotide, e.g., an HTT oligonucleotide, comprises a SNP-pairing base at Position P13. In some embodiments, an oligonucleotide, e.g., an HTT oligonucleotide, comprises a SNP-pairing base at Position P14. In some embodiments, an oligonucleotide, e.g., an HTT oligonucleotide, comprises a SNP-pairing base at Position P15. In some embodiments, an oligonucleotide, e.g., an HTT oligonucleotide, comprises a SNP-pairing base at Position P16. In some embodiments, an oligonucleotide, e.g., an HTT oligonucleotide, comprises a SNP-pairing base at Position P17. In some embodiments, an oligonucleotide, e.g., an HTT oligonucleotide, comprises a SNP-pairing base at Position P18. In some embodiments, an oligonucleotide, e.g., an HTT oligonucleotide, comprises a SNP-pairing base at Position P19. In some embodiments, an oligonucleotide, e.g., an HTT oligonucleotide, comprises a SNP-pairing base at Position P20. In some embodiments, an oligonucleotide, e.g., an HTT oligonucleotide, comprises a SNP-pairing base at Position P21. In some embodiments, an oligonucleotide, e.g., an HTT oligonucleotide, comprises a SNP-pairing base at Position P22. In some embodiments, an oligonucleotide, e.g., an HTT oligonucleotide, comprises a SNP-pairing base at Position P23. In some embodiments, an oligonucleotide, e.g., an HTT oligonucleotide, comprises a SNP-pairing base at Position P24. In some embodiments, an oligonucleotide, e.g., an HTT oligonucleotide, comprises a SNP-pairing base at Position P25. In some embodiments, an oligonucleotide, e.g., an HTT oligonucleotide, comprises a SNP-pairing base at Position P26. In some embodiments, an oligonucleotide, e.g., an HTT oligonucleotide, comprises a SNP-pairing base at Position P27. In some embodiments, an oligonucleotide, e.g., an HTT oligonucleotide, comprises a SNP-pairing base at Position P28. In some embodiments, an oligonucleotide, e.g., an HTT oligonucleotide, comprises a SNP-pairing base at Position P29. In some embodiments, an oligonucleotide, e.g., an HTT oligonucleotide, comprises a SNP-pairing base at Position P30.

In some embodiments, an HTT oligonucleotide comprises a base capable of basepairing to a SNP in an HTT mRNA at Position P3 (of the HTT oligonucleotide, wherein the position is counted as a number of bases counting from 5′ to 3′). Non-limiting examples of such an oligonucleotide include but are not limited to: WV-2023, and WV-2057.

In some embodiments, an HTT oligonucleotide comprises a base capable of basepairing to a SNP in an HTT mRNA at Position P4. Non-limiting examples of such an oligonucleotide include but are not limited to: WV-2024, WV-2025, WV-2058, and WV-2059.

In some embodiments, an HTT oligonucleotide comprises a base capable of basepairing to a SNP in an HTT mRNA at Position P5. Non-limiting examples of such an oligonucleotide include but are not limited to: WV-2026, WV-2027, WV-2060, and WV-2061.

In some embodiments, an HTT oligonucleotide comprises a base capable of basepairing to a SNP in an HTT mRNA at Position P6. Non-limiting examples of such an oligonucleotide include but are not limited to: WV-2028, WV-2029, WV-2062, and WV-2063.

In some embodiments, an HTT oligonucleotide comprises a base capable of basepairing to a SNP in an HTT mRNA at Position P7. Non-limiting examples of such an oligonucleotide include but are not limited to: WV-2030, WV-2031, WV-2064, and WV-2065.

In some embodiments, an HTT oligonucleotide comprises a base capable of basepairing to a SNP in an HTT mRNA at Position P8. Non-limiting examples of such an oligonucleotide include but are not limited to: WV-2032, WV-2033, WV-2066, and WV-2067.

In some embodiments, an HTT oligonucleotide comprises a base capable of basepairing to a SNP in an HTT mRNA at Position P9. Non-limiting examples of such an oligonucleotide include but are not limited to: WV-2034, WV-2035, WV-2068, and WV-2069.

In some embodiments, an HTT oligonucleotide comprises a base capable of basepairing to a SNP in an HTT mRNA at Position P10. Non-limiting examples of such an oligonucleotide include but are not limited to: WV-2036, WV-2037, WV-2038, WV-2070, WV-2071, and WV-2072.

In some embodiments, an HTT oligonucleotide comprises a base capable of basepairing to a SNP in an HTT mRNA at Position P11. Non-limiting examples of such an oligonucleotide include but are not limited to: WV-2039, WV-2040, WV-2041, WV-2042, WV-2073, WV-2074, WV-2075, and WV-2076.

In some embodiments, an HTT oligonucleotide comprises a base capable of basepairing to a SNP in an HTT mRNA at Position P12. Non-limiting examples of such an oligonucleotide include but are not limited to: WV-2043, WV-2044, WV-2045, WV-2046, WV-2077, WV-2078, WV-2079, and WV-2080.

In some embodiments, an HTT oligonucleotide comprises a base capable of basepairing to a SNP in an HTT mRNA at Position P13. Non-limiting examples of such an oligonucleotide include but are not limited to: WV-2047, WV-2048, WV-2049, WV-2050, WV-2081, WV-2082, WV-2083, and WV-2084.

In some embodiments, an HTT oligonucleotide comprises a base capable of basepairing to a SNP in an HTT mRNA at Position P14. Non-limiting examples of such an oligonucleotide include but are not limited to: WV-2051, WV-2052, WV-2053, WV-2085, and WV-2087.

In some embodiments, an HTT oligonucleotide comprises a base capable of basepairing to a SNP in an HTT mRNA at Position P15. Non-limiting examples of such an oligonucleotide include but are not limited to: WV-2054, WV-2055, WV-2088, and WV-2089.

In some embodiments, an HTT oligonucleotide comprises a base capable of basepairing to a SNP in an HTT mRNA at Position P16. Non-limiting examples of such an oligonucleotide include but are not limited to: WV-2056, and WV-2090.

In some embodiments, an HTT oligonucleotide comprises a BrdU. Non-limiting examples of such an oligonucleotide include: WV-1235, WV-1788, WV-1789, WV-1790, WV-2022, and WV-1234.

Data related to the efficacy of various HTT oligonucleotides which target various HTT SNPs are shown in the Examples herein and in WO2017015555 and WO2017192664.

Sequences, data and other information related to these various oligonucleotides, including WV-905, WV-911, WV-917, WV-931, WV-937, WV-944, WV-945, WV-945, WV-1085, WV-1086, WV-1087, WV-1088, WV-1089, WV-1090, WV-1091, WV-1092, WV-1497, WV-2063, WV-2067, WV-2069, WV-2072, WV-2076, WV-2077, WV-2416, WV-2417, WV-2418, WV-2419, WV-2589, WV-2590, WV-2591, WV-2592, WV-2593, WV-2594, WV-2595, WV-2596, WV-2597, WV-2598, WV-2599, WV-2600, WV-2601, WV-2602, WV-2603, WV-2604, WV-2605, WV-2606, WV-2607, WV-2608, WV-2609, WV-2610, WV-2611, WV-2612, WV-2614, WV-2615, WV-2616, WV-2617, WV-2618, WV-2619, WV-2620, WV-2671, WV-2672, WV-2673, and WV-2675, are provided herein and, for example, in WO2017015555 and WO2017192664.

In some embodiments, the present disclosure pertains to any oligonucleotide comprising a sequence of any oligonucleotide or comprising a span of 10 or more consecutive bases of the sequence of any oligonucleotide disclosed herein or in WO2017015555 or WO2017192664, wherein any one or more bases is replaced by inosine.

In some embodiments, the present disclosure pertains to any oligonucleotide comprising a sequence of any oligonucleotide or comprising a span of 10 or more consecutive bases of the sequence of any oligonucleotide disclosed herein or in WO2017015555; WO2017192664; WO0201200366; WO2011/034072; WO2014/010718; WO2015/108046; WO2015/108047; WO2015/108048; WO 2011/005761; WO 2011/108682; WO 2012/039448; WO 2018/067973; WO2005/028494; WO2005/092909; WO2010/064146; WO2012/073857; WO2013/012758; WO2014/010250; WO2014/012081; WO2015/107425; WO2017/015555; WO2017/015575; WO2017/062862; WO2017/160741; WO2017/192664; WO2017/192679; WO2017/210647; WO2018/022473; or WO2018/098264, wherein any one or more bases is replaced by inosine.

Phasing

Various techniques can be used to determine if a particular SNP allele is on the same chromosome as a disease-associated sequence, e.g., CAG repeat expansion for HTT. Typically, if the SNP allele and the CAG repeat expansion are on the same chromosome, an HTT oligonucleotide that targets that SNP allele can also “target” the disease-associated CAG repeat expansion, thereby allowing a decrease in the expression, level and/or activity of the HTT allele with the disease-associated mutation. In such a way, for example, an HTT oligonucleotide can be used in a treatment for an HTT-related disorder such as Huntington's Disease. An HTT oligonucleotide targeting a SNP can thus preferentially decrease the expression, level and/or activity of a mutant allele of HTT compared to the wild-type allele.

Humans, among other living things, are diploid, and determining the linkage of alleles of genetic loci on the same or different chromosomes is desirable for phasing techniques. The sequences on corresponding chromosomes are known as haplotypes. The process of determining which alleles are on which chromosomes is known as phasing, halpotype phasing or haplotyping. Phasing information is useful in patient stratification, forensics and various other applications in the treatment of HTT-related diseases and disorders such as Huntington's Diseases. For additional general information about phasing, see, for example: Twehey et al. 2011 Nat. Rev. Genet. 12: 215-223; and Glusman et al. 2014 Genome Med. 6:73.

Phasing data can be important in allele-specific therapies for diseases such as Huntington's Disease. In some diseases, a genetic lesion such as a deleterious repeat, deletion, insertion, inversion or other mutation has been identified, such as an expanded CAG repeat expansion in mutant (and disease-associated) HTT alleles. In some patients, one allele of a gene such as HTT can comprise a disease-associated mutation at a genetic locus, while the other allele is normal, wild-type or otherwise not disease-associated. In some embodiments, an allele-specific therapy can target an allele of HTT comprising a disease-associated mutation, but not the corresponding wild-type allele. In some embodiments, an allele-specific therapy can target an HTT allele comprising a disease-associated mutation at a particular locus, such as a CAG repeat expansion (or expanded CAG tract), but not by directly targeting the locus, but rather by targeting a different locus on the mutant allele. As a non-limiting example, an allele-specific therapy can target an allele comprising a disease-associated mutation at a locus by targeting a different locus in the same allele, such as a SNP (single nucleotide polymorphism) in the same gene.

As a non-limiting example, some disease-associated genetic lesions may be difficult to target or otherwise not readily amenable to targeting. As a non-limiting example, some genes such as mutant HTT comprise repeats (e.g., trinucleotide or tetranucleotide repeats); in some cases, such as Huntington's Disease, a small number of repeats is not disease-associated, but an abnormally large number of repeats, or a repeat expansion, is disease-associated. Because the repeats exist on both the wild-type and mutant alleles, it may be difficult to target the disease-associated repeats directly. However, if a particular SNP variant exists on the same allele as the disease-associated repeat expansion but not on the wild-type allele, that SNP variant can be used to target an allele-specific therapy which targets the mutant allele but not the wild-type allele.

As a non-limiting example, phasing data for an individual indicates if a particular SNP is in phase (e.g., on the same chromosome) as the lesion and thus that SNP can be targeted with a therapeutic nucleic acid. The therapeutic can then target the mutant gene, while not targeting the wild-type allele. Obtaining the phasing data to target only the mutant allele can be especially useful if expression of the wild-type allele is essential.

As another non-limiting example, phasing information is useful if it is known that an individual has both a wild-type and a mutant allele of each of two genetic loci on the same gene. Phasing information will reveal if both copies of the gene each have one mutant allele, or if one copy of the gene has two mutations, while the other is wild-type at both alleles.

In some embodiments, the present disclosure presents, inter alia, various methods for phasing genetic loci on a nucleic acid template. As non-limiting examples, the present disclosure presents methods for phasing a genetic locus such as a genetic lesion (such as an inversion, fusion, deletion, insertion or other mutation) and another genetic locus (such as a SNP) on a chromosome; the two genetic loci can be in the same gene, or in different genes.

In a non-limiting example, an example patient may have Huntington's Disease, which is linked to a mutation in the Huntingtin gene (HTT) comprising an excessive number of repeats (e.g., a repeat expansion) of the sequence CAG. In some embodiments, the patient may be under consideration for treatment with an allele-specific therapeutic (e.g., an antisense oligonucleotide or RNAi agent) which recognizes a particular allelic variant of a genetic locus in the HTT gene (which is outside the repeat expansion), as a non-limiting example, a SNP. If phasing reveals that the same chromosome of the patient comprises both the repeat expansion and the particular allelic variant of a genetic locus (e.g., a SNP) recognized by the allele-specific therapeutic, then the patient is eligible for treatment with the allele-specific therapeutic.

Various methods for phasing are known in the art, including but not limited to those described in: WO2018/022473; and Berger et al. 2015 Res. Comp. Mol. Biol. 9029: 28-29; Castel et al. 2015 Genome Biol. 16: 195; Castel et al. 2016 phASER: Long range phasing and haplotypic expression from RNA sequencing, doi: http://dx.doi.org/10.1101/039529; Delaneau et al. 2012 Nat. Methods 9: 179-181; Garg et al. 2016 Read-Based Phasing of Related Individuals; Hickey et al. 2011 Genet. Select. Evol. 43:12; Kuleshov et al. 2014 Nat. Biotech. 32: 261-266; Laver et al. 2016 Nature Scientific Reports | 6:21746 | DOI: 10.1038/srep21746; O'Connell et al. 2014 PLoS ONE 10: e1004234; Regan et al. 2015 PloS ONE 10: e0118270; Roach et al. 2011 Am. J. Hum. Genet. 89: 382-397; and Yang et al. 2013 Bioinformatics 29: 2245-2252. In some embodiments, sequencing, particularly sequencing that can produce long single reads, can be utilized for phasing.

Pan-Specific HTT Oligonucleotides

In some embodiments, an HTT oligonucleotide reduces expression, level, and/or activity of both mutant and wild-type HTT alleles or products thereof without significant selectivity. In some embodiments, an HTT oligonucleotide does not target a region comprising a SNP; e.g., the HTT oligonucleotide is completely complementary to a sequence in an HTT gene or mRNA which is present in all, essentially all, or nearly all human beings. Such an HTT can be considered as a pan-specific HTT oligonucleotide, and it cannot distinguish between the wild-type and mutant alleles of HTT, but may be useful in sufficiently lowering the expression, level and/or activity of the mutant HTT allele (while, in at least some cases, concomitantly lowering the expression, level and/or activity of the wild-type HTT allele). In some embodiments, a pan-specific HTT oligonucleotide is capable of mediating a decrease in the expression, level and/or activity of a mutant HTT gene or a gene product thereof which is sufficient to ameliorate, prevent, or delay the onset of Huntington's Disease or at least one symptom thereof, while simultaneously the pan-specific HTT oligonucleotide does not decrease the expression, level and/or activity of the wild-type gene or a gene product enough to cause a deleterious effect in the subject or patient.

Example reductions in levels, activities and/or expression of an HTT target gene or a gene product thereof as mediated by various HTT oligonucleotides, some of which are pan-specific, are described herein.

In some embodiments, an HTT oligonucleotide does not target a SNP. In some embodiments, a base sequence does not comprise a SNP.

In some embodiments, an HTT oligonucleotide has a base sequence which is not characterized by a known SNP; in some embodiments, such an oligonucleotide can be capable of knocking down both wild-type and mutant HTT, and in some embodiments, such an oligonucleotide is a pan-specific oligonucleotide.

A non-limiting example of a pan-specific oligonucleotide is an HTT oligonucleotide having a base sequence which is or comprises the sequence CTCAGTAACATTGACACCAC, or a span thereof (e.g., 10 contiguous bases), and which does not comprise a SNP in its base sequence. Non-limiting examples of an oligonucleotide having the base sequence of CTCAGTAACATTGACACCAC include: WV-1789, WV-1790, and WV-9679.

Another oligonucleotide known in the art having the same base sequence as CTCAGTAACATTGACACCAC is ISIS HuASO, 5′-CTCAGtaacattgacACCAC-3′, with capitalized nucleotides containing 2′-O-(2-methoxy)ethyl modifications, and non-capitalized nucleotides containing 2′-deoxy, as described in Kordasiewicz et al. 2012 Neuron 74(6): 1031-44. An oligonucleotide having this base sequence is also described in Southwell et al. 2018 Science Translational Medicine Vol. 10, Issue 461, eaar3959.

Pan-specific HTT oligonucleotides having the base sequences of CTCGACTAAAGCAGGATTTC, CCTGCATCAGCTTTATTTGT, and TCTCTATTGCACATTCCAAG were reported in Southwell et al. 2014 Mol. Ther. 22: 2093-2106. In some embodiments, the present disclosure pertains to a pan-specific HTT oligonucleotide which has a base sequence which is or comprises CTCGACTAAAGCAGGATTTC, CCTGCATCAGCTTTATTTGT, or TCTCTATTGCACATTCCAAG, or a span thereof (e.g., 10 contiguous bases) and does not comprise a SNP. In any sequence described herein, each T can be independently substituted with U and vice versa.

In some embodiments, the present disclosure pertains to an oligonucleotide composition comprising a plurality of oligonucleotides, wherein the oligonucleotides are pan-specific HTT oligonucleotides which comprise at least one chirally controlled internucleotidic linkage. In some embodiments, a chirally controlled internucleotidic linkage is a chirally controlled phosphorothioate internucleotidic linkage. In some embodiments, a chirally controlled internucleotidic linkage is a Sp chirally controlled phosphorothioate internucleotidic linkage. In some embodiments, a chirally controlled internucleotidic linkage is a Rp chirally controlled phosphorothioate internucleotidic linkage. In some embodiments, the oligonucleotides comprise at least one Sp chirally controlled phosphorothioate internucleotidic linkage, and at least one Rp chirally controlled internucleotidic linkage.

### Metabolites and Shortened Versions of Oligonucleotides

In some embodiments, an oligonucleotide, e.g., an HTT oligonucleotide, corresponds to a metabolite produced by cleavage (e.g., enzymatic cleavage by a nuclease) of a longer oligonucleotide, e.g., a longer HTT oligonucleotide. In some embodiments, the present disclosure pertains to an HTT oligonucleotide which corresponds to a metabolite produced by the cleavage of an HTT oligonucleotide described herein. In some embodiments, the present disclosure pertains to an HTT oligonucleotide which corresponds to a portion, or fragment of an HTT oligonucleotide disclosed herein.

Several experiments were performed wherein an oligonucleotide was incubated in vitro in the presence of any of various substances comprising nucleases. In various experiments, such substances include brain homogenate, cerebrospinal fluid or plasma from Sprague-Dawley rat or Cynomolgus monkey. Plasma was heparinized. Oligonucleotides were incubated for various time points (e.g., 0, 1, 2, 3, 4 or 5 days for brain tissue homogenate, with a pre-incubation period of 0, 1 or 2 days; 0, 1, 2, 4, 8, 16, 24 or 48 hrs for cerebrospinal fluid; or 0, 1, 2, 4, 8, 16 or 24 hrs for plasma). Pre-incubation indicates that the homogenate is incubated at 37 degrees C. for 0, 24 or 48 hrs to activate the enzymes before adding the oligonucleotide. Final concentration and volume of oligonucleotides was 20 μM in 200 μl. Products produced by cleavage of the oligonucleotides were analyzed by LC/MS.

One oligonucleotide has a length of 20 bases and was tested in rat brain homogenate, yielding major metabolites which were truncated at the 5′ end by 4, 10, 11, 12, or 13 bases, leaving metabolites representing the 3′ end of the oligonucleotide and which were 16, 10, 9, 8 or 7 bases long, respectively. This oligonucleotide also produced a metabolite which was a 5′ fragment which was 12 bases long (truncated at the 3′ end by 8 bases). A second oligonucleotide has a length of 20 bases and was tested in rat brain homogenate, yielding major metabolites which were truncated at the 3′ end by 4, 8, 9 or 10 bases, leaving metabolites representing the 5′ end of the oligonucleotide and which were 16, 12, 11 or 10 bases long, respectively. The two tested oligonucleotides comprise internucleotidic linkages which are phosphodiesters, phosphorothioates in the Rp configuration, and phosophorothioates in the Sp configuration. In general, phosphodiesters were more labile than either the phosphorothioate in the Rp configuration or the phosphorothioate in the Sp configuration. In some cases, a metabolite of an oligonucleotide represented the product of a cleavage at a natural phosphate linkage.

In some embodiments, the present disclosure pertains to an oligonucleotide which corresponds to a metabolite of an oligonucleotide, e.g., an HTT oligonucleotide, disclosed herein. In some embodiments, the present disclosure pertains to an oligonucleotide which is 1, 2, 3, 4, 5, 6, 7, 8, 9, 10, 11, 12, 13, or more bases shorter than an oligonucleotide disclosed herein. In some embodiments, the present disclosure pertains to an oligonucleotide which has a base sequence which is 1, 2, 3, 4, 5, 6, 7, 8, 9, 10, 11, 12, 13, or more bases shorter than that of an oligonucleotide disclosed herein.

In some embodiments, a metabolite is designated as 3′-N-#, or 5′-N-#, wherein the # indicates the number of bases removed, and the 3′ or 5′ indicates which end of the molecule from which the bases were deleted. For example, 3′-N-1 indicates a fragment or metabolite wherein 1 base was removed from the 3′ end.

In some embodiments, the present disclosure perhaps to an oligonucleotide which corresponds to a fragment or metabolite of an oligonucleotide disclosed herein, wherein the fragment or metabolite can be described as corresponding to 3′-N-1, 3′-N-2, 3′-N-3, 3′-N-4, 3′-N-5, 3′-N-6, 3′-N-7, 3′-N-8, 3′-N-9, 3′-N-10, 3′-N-11, 3′-N-12, 5′-N-1, 5′-N-2, 5′-N-3, 5′-N-4, 5′-N-5, 5′-N-6, 5′-N-7, 5′-N-8, 5′-N-9, 5′-N-10, 5′-N-11, or 5′-N-12 of an oligonucleotide described herein.

In some embodiments, the present disclosure pertains to an oligonucleotide which is 1, 2, 3, 4, 5, 6, 7, 8, 9, 10, 11, 12, 13, or more bases shorter on the 5′ end than an oligonucleotide disclosed herein. In some embodiments, the present disclosure pertains to an oligonucleotide which has a base sequence which is 1, 2, 3, 4, 5, 6, 7, 8, 9, 10, 11, 12, 13, or more bases shorter on the 5′ end than that of an oligonucleotide disclosed herein. In some embodiments, the present disclosure pertains to an oligonucleotide which is 1, 2, 3, 4, 5, 6, 7, 8, 9, 10, 11, 12, 13, or more bases shorter on the 3′ end than an oligonucleotide disclosed herein. In some embodiments, the present disclosure pertains to an oligonucleotide which has a base sequence which is 1, 2, 3, 4, 5, 6, 7, 8, 9, 10, 11, 12, 13, or more bases shorter on the 3′ end than that of an oligonucleotide disclosed herein.

In some embodiments, the present disclosure pertains to an which corresponds to a metabolite of an oligonucleotide, wherein the metabolite is truncated on the 5′ and/or 3′ end relative to the oligonucleotide disclosed herein. In some embodiments, the present disclosure pertains to an which corresponds to a metabolite of an oligonucleotide, wherein the metabolite is truncated on both the 5′ and 3′ end relative to the oligonucleotide disclosed herein. In some embodiments, the present disclosure pertains to an oligonucleotide which is 1, 2, 3, 4, 5, 6, 7, 8, 9, 10, 11, 12, 13, or more total bases shorter on the 5′ and/or 3′ end than an oligonucleotide disclosed herein. In some embodiments, the present disclosure pertains to an oligonucleotide which has a base sequence which is 1, 2, 3, 4, 5, 6, 7, 8, 9, 10, 11, 12, 13, or more bases total shorter on the 5′ and/or 3′ end than that of an oligonucleotide disclosed herein.

In some embodiments, the present disclosure pertains to an oligonucleotide which would be represented by a product of cleavage of an oligonucleotide disclosed herein, which is cleaved at a phosphodiester. In some embodiments, the present disclosure pertains to an oligonucleotide which would be represented by a product of cleavage of an oligonucleotide disclosed herein, if such an oligonucleotide were cleaved at a phosphorothioate in the Rp configuration. In some embodiments, the present disclosure pertains to an oligonucleotide which would be represented by a product of cleavage of an oligonucleotide disclosed herein, if such an oligonucleotide were cleaved at a phosphorothioate in the Rp configuration.

### Characterization and Assessment

In some embodiments, properties and/or activities of HTT oligonucleotides and compositions thereof can be characterized and/or assessed using various technologies available to those skilled in the art, e.g., biochemical assays (e.g., RNase H assays), cell based assays, animal models, clinical trials, etc.

In some embodiments, a method of identifying and/or characterizing an oligonucleotide composition, e.g., an HTT oligonucleotide composition, comprises steps of:
providing at least one composition comprising a plurality of oligonucleotides; and
assessing delivery relative to a reference composition.

In some embodiments, the present disclosure provides a method of identifying and/or characterizing an oligonucleotide composition, e.g., an HTT oligonucleotide composition, comprises steps of:
providing at least one composition comprising a plurality of oligonucleotides; and
assessing cellular uptake relative to a reference composition.

In some embodiments, the present disclosure provides a method of identifying and/or characterizing an oligonucleotide composition, e.g., an HTT oligonucleotide composition, comprises steps of:
providing at least one composition comprising a plurality of oligonucleotides; and
assessing reduction of transcripts of a target gene and/or a product encoded thereby relative to a reference composition.

In some embodiments, properties and/or activities of oligonucleotides, e.g., HTT oligonucleotides, and compositions thereof are compared to reference oligonucleotides and compositions thereof, respectively.

In some embodiments, a reference oligonucleotide composition is a stereorandom oligonucleotide composition. In some embodiments, a reference oligonucleotide composition is a stereorandom composition of oligonucleotides of which all internucleotidic linkages are phosphorothioate. In some embodiments, a reference oligonucleotide composition is a DNA oligonucleotide composition with all phosphate linkages. In some embodiments, a reference oligonucleotide composition is otherwise identical to a provided chirally controlled oligonucleotide composition except that it is not chirally controlled. In some embodiments, a reference oligonucleotide composition is otherwise identical to a provided chirally controlled oligonucleotide composition except that it has a different pattern of stereochemistry. In some embodiments, a reference oligonucleotide composition is similar to a provided oligonucleotide composition except that it has a different modification of one or more sugar, base, and/or internucleotidic linkage, or pattern of modifications. In some embodiments, an oligonucleotide composition is stereorandom and a reference oligonucleotide composition is also stereorandom, but they differ in regards to sugar and/or base modification(s) or patterns thereof.

In some embodiments, a reference composition is a composition of oligonucleotides having the same base sequence and the same chemical modifications. In some embodiments, a reference composition is a composition of oligonucleotides having the same base sequence and the same pattern of chemical modifications. In some embodiments, a reference composition is a non-chirally controlled (or stereorandom) composition of oligonucleotides having the same base sequence and chemical modifications. In some embodiments, a reference composition is a non-chirally controlled (or stereorandom) composition of oligonucleotides of the same constitution but is otherwise identical to a provided chirally controlled oligonucleotide composition.

In some embodiments, the suffix “r” is appended to the designation of a stereorandom oligonucleotide composition; e.g., WV-2614, which is stereorandom, is also designated WV-2614r. In some embodiments, the suffix “p” is appended to the designation of a chirally-controlled (or stereopure) oligonucleotide composition; e.g., WV-2599, which is stereopure, is also designated WV-2599p. The suffixes “r” and “p” are optional.

In some embodiments, a reference composition is a composition of oligonucleotides having the same base sequence but different chemical modifications, including but not limited to chemical modifications described herein. In some embodiments, a reference composition is a composition of oligonucleotides having the same base sequence but different patterns of internucleotidic linkages and/or stereochemistry of internucleotidic linkages and/or chemical modifications.

Various methods are known in the art for detection of gene products, the expression, level and/or activity of which may be altered after introduction or administration of a provided oligonucleotide. For example, transcripts and their knockdown can be detected and quantified with qPCR, and protein levels can be determined via Western blot.

In some embodiments, assessment of efficacy of oligonucleotides can be performed in biochemical assays or in vitro in cells. In some embodiments, provided oligonucleotides can be introduced to cells via various methods available to those skilled in the art, e.g., gymnotic delivery, transfection, lipofection, etc.

In some embodiments, an HTT oligonucleotide is tested in a cell or animal model of HD.

In some embodiments, a cell model of HD is a cell comprising a wild-type and/or mutant HTT gene. In some embodiments, a cell model or animal model which comprises a wild-type HTT gene can be used as a control in an experiment involving the knockdown of a mutant HTT gene in a corresponding cell model or animal model. In some embodiments, wherein an HTT oligonucleotide is designed to knock down both wild-type and mutant HTT alleles (e.g., a pan-specific HTT oligonucleotide), a cell model and/or animal model comprising a wild-type and/or mutant HTT allele can be used to evaluate the ability of the HTT oligonucleotide to knock down HTT.

In some embodiments, a cell model of HD is an iCell neuron or iPSC-derived neuron.

In some embodiments, a cell model of HD is a PC12 cell expressing the mutant huntingtin gene.

In some embodiments, a cell model of HD is a HD patient fibroblast.

In some embodiments, a cell model of HD is a PC6-3 rat pheochromocytoma cell, which was reportedly co-transfected with CMV-human HTT (37Qs) and U6 siRNA hairpin plasmids. See, for example: U.S. Ser. No. 10/072,264.

In some embodiments, a cell model of HD is a striatal cell established from Hdh Q111 knock-in mice, which bear 111 CAG repeats inserted into the mouse huntingtin locus. See, for example: Trettel et al. Human Mol. Genet., 2000, 9, 2799-2809.

In some embodiments, a cell model of HD is a mouse striatum cell line with wild-type huntingtin, STHdhQ7/7 (Q7/7), and/or mutant huntingtin, STHdhQ111/111 (Q111/111).

In some embodiments, a cell model of HD is a mouse striatum cell line with wild-type huntingtin, STHdhQ7/7 (Q7/7), and mutant huntingtin, STHdhQ111/111 (Q111/111).

In some embodiments, a cell model comprises: a construct spanning exons 1-3 of mouse HTT containing a 79 CAG repeat expansion, the mouse equivalent of N171-82Q.

Many technologies for assessing activities and/or properties of oligonucleotides in animals are known and practiced by those skilled in the art and can be utilized in accordance with the present disclosure. In some embodiments, evaluation of an oligonucleotide can be performed in an animal. Various animals may be used to assess properties and activities of provided oligonucleotides and compositions thereof.

Identification of the HTT gene has allowed for the development of animal models of the disease, including transgenic mice carrying mutated human or mouse forms of the gene. Models include mice carrying a fragment of the human gene, typically the first one or two exons, which contains the glutamine expansion (or the wild-type equivalent), in addition to the undisrupted wild-type, endogenous, mouse gene; mice carrying the full length human huntingtin with an expanded glutamine repeat region, again with the endogenous mouse gene; and mice with pathogenic CAG repeats inserted into the CAG repeat region. All of the models have at least some shared features with the human disease. These mice have allowed for the testing of a number of different therapeutic agents for the prevention, amelioration and treatment of HD (see, e.g., Hersch and Ferrante, 2004. NeuroRx. 1:298-306) using a number of endpoints. The compounds are believed to function by a number of different mechanisms including transcription inhibition, caspace inhibition, histone deacetylase inhibition, antioxidant, huntingtin inhibition/antioxidant, biogenergetic/antioxidant, antiexcitotoxic, and antiapoptotic.

Various animal models of HD have been reported in the literature. These include, as non-limiting examples, those reported in: Diaz-Hernandez et al. 2005. J. Neurosci. 25:9773-81; Wang et al. 2005. Neurosci. Res. 53:241-9; Machida et al. 2006. Biochem. Biophys. Res. Commun. 343:190-7; Harper et al. 2005. PNAS 102:5820-25; or Rodrigues-Lebron et al. 2005. Mol. Ther. 12:618-33; Mangiarini L. et al., Cell. 1996 November; 87(3):493-506; and Southwell et al. Science Translational Medicine 3 Oct. 2018: Vol. 10, Issue 461, eaar3959; or Meade et al., J. Comp. Neurol. 449:241-269, 2002.

For information related to animal models and other experimental procedures related to HTT, see those noted herein or in the relevant art, including, for example: Hersch and Ferrante 2004 NeuroRx. 1:298-306; Diaz-Hernandez et al. 2005. J. Neurosci. 25:9773-81; Wang et al. 2005. Neurosci. Res. 53:241-9; Machida et al. 2006. Biochem. Biophys. Res. Commun. 343:190-7; Harper et al. 2005. PNAS 102:5820-25; Rodrigues-Lebron et al. 2005. Mol. Ther. 12:618-33; Nguyen et al. 2005. PNAS 102:11840-45.

In some embodiments, an animal model of HD is a mouse carrying the full length human huntingtin with an expanded glutamine repeat region, again with the endogenous mouse gene; and mice with pathogenic CAG repeats inserted into the CAG repeat region. In some embodiments, an animal model of HD is mouse model R6/2 or R6/1.

In some embodiments, an animal model of HD is a R6/2 transgenic mouse model, which reportedly has integrated into its genome 1 kilobase of the human huntingtin gene, including the 5′-UTR exon 1 and the first 262 basepairs of intron 1. See, for example: Mangiarini L. et al., Cell, 1996, 87, 493-506. This transgene reportedly has 144 CAG repeats. The transgene reportedly encodes for approximately 3% of the N-terminal region of the huntingtin protein, expression of which is driven by the human huntingtin promoter. Expression levels of this truncated version of human huntingtin protein are reportedly approximately 75% of the endogenous mouse huntingtin protein levels. The R6/2 transgenic mice reportedly exhibit symptoms of human Huntington's disease and brain dysfunction.

In some embodiments, an animal model of HD is a YAC128 transgenic mice, which reportedly harbors a yeast artificial chromosome (YAC) carrying the entire huntingtin gene, including the promoter region and 128 CAG repeats. See, for example: Hodgson J. G. et al., Human Mol. Genet., 1998, 5, 1875. This YAC reportedly expresses all but exon 1 of the human gene. These transgenic mice reportedly do not express endogenous mouse huntingtin.

In some embodiments, an animal model of HD is a Q111 mice, the endogenous mouse huntingtin gene of which reportedly has 111 CAG repeats inserted into exon 1 of the gene. See, for example: Wheeler V. C. et al., Human Mol. Genet., 8, 115-122).

In some embodiments, an animal model of HD is a Q150 transgenic mice, wherein the CAG repeat in exon 1 of the wild-type mouse huntingtin gene is reportedly replaced with 150 CAG repeats. See, for example: Li C. H. et al., Human Mol. Genet., 2001, 10, 137.

In some embodiments, an animal model of HD is a tetracycline-regulated mouse model of HD. See, for example: Yamamoto et al., Cell, 101(1), 57-66 (2000).

In some embodiments, an animal model of HD is any of the transgenic and knock-in mouse models described in: Bates et al., Curr Opin Neurol 16:465-470, 2003.

In some embodiments, an animal model of HD is a HD mouse model, wherein adding two additional exons to the transgene and restricting expression via the prion promoter reportedly led to an HD mouse model displaying important HD characteristics but with less aggressive disease progression. See, for example: Schilling et al., Hum Mol Genet 8(3):397-407, 1999; and Schilling et al., Neurobiol Dis 8:405-418, 2001.

In some embodiments, an animal model of HD is a mouse knock-in model, wherein Detloff and colleagues reportedly created a mouse knock-in model with an extension of the endogenous mouse CAG repeat to approximately 150 CAGs. This model, the CHL2 line, reportedly shows more aggressive phenotypes than prior mouse knock-in models containing few repeats. Measurable neurological deficits reportedly include clasping, gait abnormalities, nuclear inclusions and astrogliosis. Lin et al., Hum. Mol. Genet., 10(2), 137-44 (2001).

In some embodiments, a cell model or animal model (e.g., a mouse model) comprises: a construct spanning exons 1-3 of mouse HTT containing a 79 CAG repeat expansion, the mouse equivalent of N171-82Q.

In some embodiments, an animal model of HD is a Borchelt mouse model (N171-82Q, line 81) or a Detloff knock-in model, the CHL2 line.

In some embodiments, an animal model of HD is a Borchelt model, N171-82Q, which reportedly has greater than wildtype levels of RNA, but reduced amounts of mutant protein relative to endogenous HTT. N171-82Q mice reportedly show normal development for the first 1-2 months, followed by failure to gain weight, progressive incoordination, hypokinesis and tremors.

In some embodiments, an animal model of HD is a mouse Huntington's Disease (HD) model expressing mutant exon 1. See, for example: WO2018145009.

In some embodiments, an animal model of HD is a rat. See, for example: Jae K. Ryu et al. Neurobiology of Disease, Volume 16, Issue 1, June 2004, Pages 68-77; O. Isacson, Neuroscience, Volume 22, Issue 2, August 1987, Pages 481-497; and Stephan von Horsten et al., Human Molecular Genetics, Volume 12, Issue 6, 15 Mar. 2003, Pages 617-624.

In some embodiments, an animal model of HD is a monkey. See, for example: Kenya Sato and Erika Sasaki, Journal of Human Genetics, volume 63, pages 125-131 (2018); and Kittiphong Putkhao, Cloning Transgenes. 2013; 2: 1000116.

Additional documents related to the use of animal models of HD include: Ian Fyfe Nature Reviews Neurology (2018); and Kenya Sato and Erika Sasaki, Journal of Human Genetics, volume 63, pages 125-131 (2018).

In some embodiments, wherein an oligonucleotide, e.g., an HTT oligonucleotide, which targets a particular SNP variant, it may be desirable to test the oligonucleotide in a particular test animal. However, it may also be the case that the test animal may not have in its genome the complement of that SNP variant. In such a case, it may be desirable to construct an oligonucleotide which is identical to the HTT oligonucleotide to be tested except that it has a SNP variant which is complementary to the SNP variant in the test animal. Such an oligonucleotide can be termed, for example, a surrogate of the HTT oligonucleotide to be tested. In some embodiments, a provided HTT oligonucleotide is identical to any HTT oligonucleotide described herein, or any oligonucleotide which comprises at least 10 contiguous bases thereof, except that the oligonucleotide comprises a different SNP variant than that described herein.

In some embodiments, an animal model administered an oligonucleotide, e.g., an HTT oligonucleotide, can be evaluated for safety and/or efficacy.

In some embodiments, the effect(s) of administration of an oligonucleotide to an animal can be evaluated, including any effects on behavior, inflammation, and toxicity. In some embodiments, following dosing, animals can be observed for signs of toxicity including trouble grooming, lack of food consumption, and any other signs of lethargy. In some embodiments, in a mouse model of Huntington's Disease, following administration of an HTT oligonucleotide, the animals can be monitored for timing of onset of a rear paw clasping phenotype.

In some embodiments, following administration of an HTT oligonucleotide to an animal, the animal can be sacrificed and analysis of tissues or cells can be performed to determine changes in mutant or wild-type HTT, or other biochemical or other changes. In some embodiments, following necropsy, liver, heart, lung, kidney, and spleen can be collected, fixed, and processed for histopathological evaluation (standard light microscopic examination of hematoxylin and eosin-stained tissue slides).

In some embodiments, following administration of an oligonucleotide, e.g., an HTT oligonucleotide, to an animal, behavioral changes can be monitored or assessed. In some embodiments, such an basement can be performed using accelerating rotarod and open field testing. In some embodiments, rotarod analysis can be carried out using a San Diego Instruments™ (San Diego, Calif.) rodent rotarod. In some embodiments, an automated 30-minute assessment of open field behavior can also be conducted, e.g., using a Noldus Etho Vision video tracking system to record and digitize the mouse movements (Noldus Information Technology, The Netherlands). In some embodiments, software can be used to dichotomize mouse movements into lingering episodes and progression segments, and calculate further parameters for these, such as speed and acceleration. In some embodiments, following administration of an HTT oligonucleotide, a test animal can be evaluated for rotarod (RR) performance or open field parameters as distance traveled, maximum speed, number of stops of anxiety (i.e. avoiding the arena center). In some embodiments, a test animal can be used to evaluate the pharmacokinetics and pharmacodynamics of an HTT oligonucleotide.

Various effects of testing in animals described herein can also be monitored in human subjects or patients following administration of an HTT oligonucleotide.

In addition, the efficacy of an HTT oligonucleotide in a human patient can be measured by evaluating, after administration of the oligonucleotide, any of various parameters known in the art, including but not limited to the following: Total Motor Score (TMS); Symbol Digit Modalities Test (SDMT); Stroop Word Reading Test (SWRT); Total Functional Capacity (TFC) score; and/or Composite Unified Huntington's Disease Rating Scale (cUHDRS).

In some embodiments, following human treatment with an oligonucleotide, or contacting a cell or tissue in vitro with an oligonucleotide, cells and/or tissues are collected for analysis.

In some embodiments, in various cells and/or tissues, target HTT nucleic acid levels can be quantitated by methods available in the art, many of which can be accomplished with commercially available kits and materials. Such methods include, e.g., Northern blot analysis, competitive polymerase chain reaction (PCR), quantitative real-time PCR, etc. RNA analysis can be performed on total cellular RNA or poly(A)+ mRNA. Probes and primers are designed to hybridize to a nucleic acid to be detected. Methods for designing real-time PCR probes and primers are well known and widely practiced in the art. For example, to detect and quantify HTT RNA, an example method comprises isolation of total RNA (e.g., including mRNA) from a cell or animal treated with an oligonucleotide or a composition and subjecting the RNA to reverse transcription and/or quantitative real-time PCR, for example, as described herein, or in: Moon et al. 2012 Cell Metab. 15: 240-246.

In some embodiments, protein levels can be evaluated or quantitated in various methods known in the art, e.g., enzyme-linked immunosorbent assay (ELISA), Western blot analysis (immunoblotting), immunocytochemistry, fluorescence-activated cell sorting (FACS), immunohistochemistry, immunoprecipitation, protein activity assays (for example, caspase activity assays), and quantitative protein assays. Antibodies useful for the detection of mouse, rat, monkey, and human proteins are commercially available or can be generated if needed. For example, various HTT antibodies are commercially available and/or have been reported in e.g., those commercially available from LifeSpan BioSciences, Seattle, Wash.; Sigma-Aldrich, St. Louis, Mo.; etc.

Various technologies are available and/or known in the art for detecting levels of oligonucleotides or other nucleic acids. Such technologies are useful for detecting HTT oligonucleotides when administered to assess, e.g., delivery, cell uptake, stability, distribution, etc.

In some embodiments, selection criteria are used to evaluate the data resulting from various assays and to select particularly desirable oligonucleotides, e.g., desirable HTT oligonucleotides, with certain properties and activities. In some embodiments, selection criteria include an IC₅₀ of less than about 10 nM, less than about 5 nM or less than about 1 nM. In some embodiments, selection criteria for a stability assay include at least 50% stability [at least 50% of an oligonucleotide is still remaining and/or detectable] at Day 1. In some embodiments, selection criteria for a stability assay include at least 50% stability at Day 2. In some embodiments, selection criteria for a stability assay include at least 50% stability at Day 3. In some embodiments, selection criteria for a stability assay include at least 50% stability at Day 4. In some embodiments, selection criteria for a stability assay include at least 50% stability at Day 5. In some embodiments, selection criteria for a stability assay include at least 80% [at least 80% of the oligonucleotide remains] at Day 5.

In some embodiments, a target gene, e.g., HTT, is a wild-type gene. In some embodiments, a target gene comprises one or more mutations. In some embodiments, a target gene comprises a mutation associated with a disorder. In some embodiments, a mutation is a single nucleotide polymorphism (SNP). In some embodiments, base sequences of provided oligonucleotides are complementary to target sequences in transcripts comprising a mutation or SNP associated with a condition, disorder or disease. In some embodiments, provided oligonucleotides and compositions selectively reduce levels of transcripts comprising a mutation or SNP associated with a condition, disorder or disease and/or products encoded thereby relative to wild-type transcripts and/or transcripts less associated with a condition, disorder or disease and/or products encoded thereby. In many embodiments, provided oligonucleotides are complementary to transcripts comprising mutations or SNPs associated with conditions, disorders or diseases at the mutation or SNP sites while they have mismatches when hybridizing to wild-type or less associated transcripts at the sites corresponding to the mutations or SNPs. In some embodiments, a mutation or SNP is located 0, 1, 2, 3 or 4 internucleotidic linkages from a Rp or Op internucleotidic linkage when a transcript comprising the mutation or SNP is hybridized with a provided oligonucleotide.

In some embodiments, efficacy of an HTT oligonucleotide is assessed directly or indirectly by monitoring, measuring or detecting a change in a condition, disorder or disease or a biological pathway associated with HTT.

In some embodiments, efficacy of an HTT oligonucleotide is assessed directly or indirectly by monitoring, measuring or detecting a change in a biochemical phenomenon associated with Huntington's Disease (HD), such as any of: insoluble protein accumulation; huntingtin protein aggregate accumulation; neuronal aggregates in the striatum; alteration in the size and number of neuronal intranuclear inclusions and other markers of HD; alteration in regulation of DARPP-32 expression; striatal atrophy; striatal and cortical neurodegeneration; alteration of blood glucose and/or insulin levels; or neuronal loss and gliosis, particularly in the cortex and striatum.

In some embodiments, efficacy of an HTT oligonucleotide is assessed directly or indirectly by monitoring, measuring or detecting a change in a response to be affected by HTT knockdown.

In some embodiments, a provided oligonucleotide (e.g., an HTT oligonucleotide) can by analyzed by a sequence analysis to determine what other genes [e.g., genes which are not a target gene (e.g., HTT)] have a sequence which is complementary to the base sequence of the provided oligonucleotide (e.g., the HTT oligonucleotide) or which have 0, 1, 2 or more mismatches from the base sequence of the provided oligonucleotide (e.g., the HTT oligonucleotide). Knockdown, if any, by the oligonucleotide of these potential off-targets can be determined to evaluate potential off-target effects of an oligonucleotide (e.g., an HTT oligonucleotide). In some embodiments, an off-target effect is also termed an unintended effect and/or related to hybridization to a bystander (non-target) sequence or gene.

Oligonucleotides which have been evaluated and tested for efficacy in knocking down HTT have various uses, e.g., in treatment or prevention of an HTT-related condition, disorder or disease or a symptom thereof.

In some embodiments, an HTT oligonucleotide which has been evaluated and tested for its ability to provide a particular biological effect (e.g., reduction of level, expression and/or activity of an HTT target gene or a gene product thereof) can be used to treat, ameliorate and/or prevent an HTT-related condition, disorder or disease.

### HTT-Related Conditions, Disorders or Diseases

In some embodiments, provided oligonucleotides and compositions thereof are capable of providing a decrease in the expression and/or level of an HTT target gene or a gene product thereof. In some embodiments, a provided oligonucleotide or composition targets an HTT gene and is useful for treatment of HTT-related conditions, disorders or diseases. In some embodiments, the present disclosure provides oligonucleotides and compositions for preventing and/or treating HTT-related conditions, disorders or diseases. In some embodiments, the present disclosure provides methods for preventing and/or treating HTT-related conditions, disorders or diseases, comprising administering to a subject susceptible thereto or suffering therefrom a therapeutically effective amount of a provided HTT oligonucleotide or a composition thereof. HTT-related conditions, disorders or diseases are extensively described in the art.

In some embodiments, an HTT-related condition, disorder or disease is a condition, disorder or disease that is related to, caused by and/or associated with abnormal or excessive activity, level and/or expression, or abnormal tissue or inter- or intracellular distribution, of an HTT gene or a gene product thereof. In some embodiments, an HTT-related condition, disorder or disease is associated with HTT if the presence, level and/or form of transcription of an HTT region, an HTT transcript and/or a product encoded thereby correlates with incidence of and/or susceptibility to the condition, disorder or disease (e.g., across a relevant population). In some embodiments, an HTT-related condition, disorder or disease is a condition, disorder or disease in which reduction of the level, expression and/or activity of an HTT gene or a product thereof ameliorates, prevents and/or reduces the severity of the condition, disorder or disease.

Examples of HTT-related conditions, disorders or diseases include Huntington's Disease (HD), also known as Huntington's Chorea. In some embodiments, a HTT-related condition, disorder or disease is: juvenile HD, akinetic-rigid, or Westphal variant HD.

Among other things, the present disclosure provides methods of using oligonucleotides disclosed herein which are capable of targeting HTT for treating and/or manufacturing a treatment for an HTT-related condition, disorder or disease. In some embodiments, a base sequence of an HTT oligonucleotide or a single-stranded RNAi agent can comprise or consist of a base sequence which has a specified maximum number of mismatches (e.g., 1, 2, 3, etc.) from a specified base sequence.

### Treatment of HTT-Related Conditions, Disorders or Diseases

In some embodiments, the present disclosure provides an HTT oligonucleotide which targets HTT (e.g., an HTT oligonucleotide comprising an HTT target sequence or a sequence complementary to an HTT target sequence). In some embodiments, the present disclosure provides an HTT oligonucleotide which directs target-specific knockdown of HTT. In some embodiments, the present disclosure provides an HTT oligonucleotide which directs target-specific knockdown of HTT mediated by RNaseH and/or RNA interference. Various oligonucleotides capable of targeting HTT are provided herein. In some embodiments, the present disclosure provides methods for preventing and/or treating HTT-related conditions, disorders or diseases using provided HTT oligonucleotides and compositions thereof. In some embodiments, the present disclosure provides oligonucleotides and compositions thereof for use as medicaments, e.g., for HTT-related conditions, disorders or diseases. In some embodiments, the present disclosure provides oligonucleotides and compositions thereof for use in the treatment of HTT-related conditions, disorders or diseases. In some embodiments, the present disclosure provides oligonucleotides and compositions thereof for the manufacture of medicaments for the treatment of HTT-related conditions, disorders or diseases.

In some embodiments, the present disclosure provides a method for preventing, treating or ameliorating an HTT-related condition, disorder or disease in a subject susceptible thereto or suffering therefrom, comprising administering to the subject a therapeutically effective amount of an HTT oligonucleotide or a pharmaceutical composition thereof.

In some embodiments, the present disclosure provides a method for treating or ameliorating an HTT-related condition, disorder or disease in a subject suffering therefrom, comprising administering to the subject a therapeutically effective amount of an HTT oligonucleotide or a pharmaceutical composition thereof.

In some embodiments, an HTT-related condition, disorder or disease is Huntington's Disease (HD), also known as Huntington's Chorea. In some embodiments, a HTT-related condition, disorder or disease is: juvenile HD, akinetic-rigid, or Westphal variant HD.

In some embodiments, the present disclosure provides a method for reducing HTT gene expression in a cell, comprising: contacting the cell with an HTT oligonucleotide or a composition thereof. In some embodiments, the present disclosure provides a method for reducing the level of an HTT transcript in a cell, comprising: contacting the cell with an HTT oligonucleotide or a composition thereof. In some embodiments, the present disclosure provides a method for reducing the level of an HTT protein in a cell, comprising: contacting the cell with an HTT oligonucleotide or a composition thereof. In some embodiments, provided methods selectively reduce levels of HTT transcripts and/or products encoded thereby that are related to conditions, disorders or diseases.

Reportedly, HTT is expressed in all cells, with the highest concentrations are found in the brain and testes, with moderate amounts in the liver, heart, and lungs. In various embodiments, a cell is in brain, testes, liver, heart, or lungs.

In some embodiments, the present disclosure provides a method for decreasing HTT gene expression in a mammal in need thereof, comprising administering to the mammal a nucleic acid-lipid particle comprising a provided HTT oligonucleotide or a composition thereof.

In some embodiments, the present disclosure provides a method for in vivo delivery of an HTT oligonucleotide, comprising administering to a mammal an HTT oligonucleotide or a composition thereof.

In some embodiments, a mammal is a human. In some embodiments, a mammal is afflicted with and/or suffering from an HTT-related condition, disorder or disease.

In some embodiments, a subject or patient suitable for treatment of an HTT-related condition, disorder or disease, such as Huntington's Disease (HD), can be identified or diagnosed by a health care professional. For example, for a neurological condition, disorder or disease, a physical exam may be followed by a thorough neurological exam. In some embodiments, an neurological exam may assess motor and sensory skills, nerve function, hearing and speech, vision, coordination and balance, mental status, and/or changes in mood or behavior. Example symptoms of neurological conditions, disorders or diseases, such as Huntington's Disease (HD), include weakness in the arms, legs, feet, or ankles; slurring of speech; difficulty lifting the front part of the foot and toes; hand weakness or clumsiness; muscle paralysis; rigid muscles; involuntary jerking or writing movements (chorea); involuntary, sustained contracture of muscles (dystonia); bradykinesia; loss of automatic movements; impaired posture and balance; lack of flexibility; tingling parts in the body; electric shock sensations that occur with movement of the head; twitching in arm, shoulders, and tongue; difficulty swallowing; difficulty breathing; difficulty chewing; partial or complete loss of vision; double vision; slow or abnormal eye movements; tremor; unsteady gait; fatigue; loss of memory; dizziness; difficulty thinking or concentrating; difficulty reading or writing; misinterpretation of spatial relationships; disorientation; depression; anxiety; difficulty making decisions and judgments; loss of impulse control; difficulty in planning and performing familiar tasks; aggressiveness; irritability; social withdrawal; mood swings; dementia; change in sleeping habits; wandering; and/or change in appetite.

In some embodiments, a symptom of Huntington's Disease is any of: insoluble protein accumulation; huntingtin protein aggregate accumulation; neuronal aggregates in the striatum; alteration in the size and number of neuronal intranuclear inclusions and other markers of HD; alteration in regulation of DARPP-32 expression; striatal atrophy; striatal and cortical neurodegeneration; alteration of blood glucose and/or insulin levels; or neuronal loss and gliosis, particularly in the cortex and striatum.

In some embodiments, a symptom of Huntington's Disease is any of: behavioral and neuropathological abnormalities; in test animals, altered rotarod performance; reduction of weight loss; alteration of lifespan; behavioral disturbance; emotional, motor and cognitive alterations or impairment; depression; irritability; involuntary movements (chorea); choreiform movements; impaired coordination; excessive spontaneous movements which are irregularly timed, randomly distributed and abrupt; bradykinesia; dystonia; seizures; rigidity; ocularmotor dysfunction; tremor; fine motor incoordination; dysathria; dysphagia; subcortical dementia; progressive dementia; or psychiatric disturbance.

In some embodiments, a provided oligonucleotide or a composition thereof prevents, treats, ameliorates, or slows progression of an HTT-related condition, disorder or disease, or at least one symptom of an HTT-related condition, disorder or disease.

In some embodiments, a method of the present disclosure is for the treatment of Huntington's Disease in a subject wherein the method comprises administering to a subject a therapeutically effective amount of an HTT oligonucleotide or a pharmaceutical composition thereof.

In some embodiments, a provided method reduces at least one symptom of Huntington's Disease wherein the method comprises administering to a subject a therapeutically effective amount of an HTT oligonucleotide or a pharmaceutical composition thereof.

In some embodiments, the present disclosure provides a method for the treatment or reduction of at least one point in severity of Huntington's Disease or reduction in medical consequences of non-alcoholic steatohepatitis in a subject, comprising administering to a subject a therapeutically effective amount of an HTT oligonucleotide or a pharmaceutical composition thereof.

In some embodiments, the present disclosure provides a method for treating and/or ameliorating one or more symptoms associated with an HTT-related condition, disorder or disease in a mammal in need thereof, the method comprising administering to the mammal a therapeutically effective amount of an HTT oligonucleotide or a composition thereof. In some embodiments, the present disclosure provides a method for reducing susceptibility to an HTT-related condition, disorder or disease in a mammal in need thereof, the method comprising: administering to the mammal a therapeutically effective amount of an HTT oligonucleotide or a composition thereof. In some embodiments, the present disclosure provides a method for preventing or delaying the onset of an HTT-related condition, disorder or disease in a mammal in need thereof, the method comprising: administering to the mammal a therapeutically effective amount of an HTT oligonucleotide or a composition thereof. In some embodiments, the present disclosure provides a method for treating and/or ameliorating one or more symptoms associated with an HTT-related condition, disorder or disease in a mammal in need thereof, the method comprising: administering to the mammal a therapeutically effective amount of a nucleic acid-lipid particle comprising an HTT oligonucleotide. In some embodiments, the present disclosure provides a method for reducing susceptibility to an HTT-related condition, disorder or disease in a mammal in need thereof, the method comprising: administering to the mammal a therapeutically effective amount of a nucleic acid-lipid particle comprising an HTT oligonucleotide. In some embodiments, the present disclosure provides a method for preventing or delaying the onset of an HTT-related condition, disorder or disease in a mammal in need thereof, the method comprising: administering to the mammal a therapeutically effective amount of a nucleic acid-lipid particle comprising an HTT oligonucleotide. In some embodiments, a mammal is a human. In some embodiments, a mammal is afflicted with and/or suffering from an HTT-related condition, disorder or disease.

In some embodiments, administration of an HTT oligonucleotide to a patient or subject is capable of mediating any one or more of: slowing Huntington's Disease progression, delaying the onset of HD or at least one symptom thereof, improving one or more indicators of HD, and/or increasing the survival time or lifespan of the patient or subject.

In some embodiments, slowing disease progression relates to the prevention of, or delay in, a clinically undesirable change in one or more clinical parameters in an individual suffering from HD, such as those described herein. It is well within the abilities of a physician to identify a slowing of disease progression in an individual suffering from HD, using one or more of the disease assessment tests described herein. Additionally, it is understood that a physician may administer to the individual diagnostic tests other than those described herein to assess the rate of disease progression in an individual suffering from HD.

In some embodiments, delaying the onset of HD or a symptom thereof relates to delaying one or more undesirable changes in one or more indicators of HD that are negative for HD. A physician may use family history of HD or comparisons to other HD patients with similar genetic profile (e.g., number of CAG repeats) to determine an expected approximate age of HD onset to HD to determine if onset of HD is delayed.

In some embodiments, indicators of HD include parameters employed by a medical professional, such as a physician, to diagnose or measure the progression of HD, and include, without limitation, genetic testing, hearing, eye movements, strength, coordination, chorea (rapid, jerky, involuntary movements), sensation, reflexes, balance, movement, mental status, dementia, personality disorder, family history, weight loss, and degeneration of the caudate nucleus. Degeneration of the caudate nucleus is assessed via brain imaging techniques such as magnetic resonance imaging (MRI) or computed tomography (CT) scan.

In some embodiments, an improvement in an indicator of HD relates to the absence of an undesirable change, or the presence of a desirable change, in one or more indicators of HD. In one embodiment, an improvement in an indicator of HD is evidenced by the absence of a measurable change in one or more indicators of HD. In another embodiment, an improvement in an indicator of HD is evidenced by a desirable change in one or more indicators of HD.

In some embodiments, a slowing of disease progression may further comprise an increase in survival time in an individual suffering from HD. In some embodiments, an increase in survival time relates to mean increasing the survival of an individual suffering from HD, relative to an approximate survival time based upon HD progression and/or family history of HD. A physician can use one or more of the disease assessment tests described herein to predict an approximate survival time of an individual suffering from HD. A physician may additionally use the family history of an individual suffering from HD or comparisons to other HD patients with similar genetic profile (e.g., number of CAG repeats) to predict expected survival time.

In some embodiments, the present disclosure provides a method of inhibiting HTT expression in a cell, the method comprising: (a) contacting the cell with an HTT oligonucleotide; and (b) maintaining the cell produced in step (a) for a time sufficient to obtain degradation of a mRNA transcript of an HTT gene, thereby inhibiting expression of the HTT gene in the cell. In some embodiments, HTT expression is inhibited by at least 30%.

In some embodiments, the present disclosure provides a method of treating a condition, disorder or disease mediated by HTT expression comprising administering to a human suffering therefrom a therapeutically effective amount of an HTT oligonucleotide or a composition thereof. In some embodiments, administration causes a decrease in the expression, activity and/or level of an HTT transcript. In some embodiments, administration is associated with a decrease in the expression, activity and/or level of an HTT transcript. In some embodiments, administration is followed by a decrease in the expression, activity and/or level of an HTT transcript.

In some embodiments, the present disclosure provides an HTT oligonucleotide for use in a subject to treat an HTT-related condition, disorder or disease. In some embodiments, an HTT-related condition, disorder or disease is selected from Huntington's Disease.

In some embodiments, a subject is administered an oligonucleotide, e.g., an HTT oligonucleotide, or a composition thereof and an additional agent and/or method, e.g., an additional therapeutic agent and/or method. In some embodiments, an oligonucleotide or composition thereof can be administered alone or in combination with one or more additional therapeutic agents and/or treatment. When administered in combination each component may be administered at the same time or sequentially in any order at different points in time. In some embodiments, each component may be administered separately but sufficiently closely in time so as to provide the desired therapeutic effect. In some embodiments, provided oligonucleotides and additional therapeutic components are administered concurrently. In some embodiments, provided oligonucleotides and additional therapeutic components are administered as one composition. In some embodiments, at a time point a subject being administered is exposed to both provided oligonucleotides and additional components at the same time.

In some embodiments, an additional therapeutic agent or method is capable of preventing, treating, ameliorating or slowing the progress of a neurological condition, disorder or disease. In some embodiments, an additional therapeutic agent or method is capable of preventing, treating, ameliorating or slowing the progress of an HTT-related condition, disorder or disease. In some embodiments, an additional therapeutic agent or method may “indirectly” decrease the expression, activity and/or level of HTT, e.g., by knocking down a gene or gene product which can increases the expression, activity and/or level of HTT.

In some embodiments, an additional therapeutic agent is physically conjugated to an oligonucleotide, e.g., an HTT oligonucleotide. In some embodiments, an additional agent is an HTT oligonucleotide. In some embodiments, a provided oligonucleotide is physically conjugated with an additional agent which is an HTT oligonucleotide. In some embodiments, additional agent oligonucleotides have base sequences, sugars, nucleobases, internucleotidic linkages, patterns of sugar, nucleobase, and/or internucleotidic linkage modifications, patterns of backbone chiral centers, etc., or any combinations thereof, as described in the present disclosure. In some embodiments, an additional oligonucleotide targets HTT. In some embodiments, an HTT oligonucleotide is physically conjugated to a second oligonucleotide which can decrease (directly or indirectly) the expression, activity and/or level of HTT, or which is useful for treating an HTT-related condition, disorder or disease. In some embodiments, a first HTT oligonucleotide is physically conjugated to a second HTT oligonucleotide, which can be identical to the first HTT oligonucleotide or not identical, and which can target a different or the same or an overlapping sequence as the first HTT oligonucleotide.

In some embodiments, an HTT oligonucleotide may be administered with one or more additional (or second) therapeutic agent for HD, e.g., a selective serotonin reuptake inhibitor, amantadine, an antiparkinsonian drug, an antipsychotic drug, benzodiazepine, mirtazapine, neuroleptic, remacemide, valproic acid, Tetrabenazine (Xenazine), an antipsychotic drug, haloperidol (Haldol), chlorpromazine, risperidone (Risperdal), quetiapine (Seroquel), a medication that may help suppress chorea, amantadine, levetiracetam (Keppra), clonazepam (Klonopin), a medication to treat a psychiatric disorder, an antidepressant, citalopram (Celexa), escitalopram (Lexapro), fluoxetine (Prozac, Sarafem), sertraline (Zoloft), Risperdal (risperidone), Haldol (haloperidol), Thorazine (chlorpromazine), an antipsychotic drug, quetiapine (Seroquel), risperidone (Risperdal), olanzapine (Zyprexa), a mood-stabilizing drug, an anticonvulsant, valproate (Depacon), carbamazepine (Carbatrol, Epitol, Tegretol), Klonopin (clonazepam), Valium (diazepam), Carbatrol (carbamazepine), Depacon (valproate), Lamictal (lamotrigine), SRX246, gene silencing therapy, a therapy intended to reduce inflammation in the brain, VX15/2503, KD3010, VX15, bexarotene, laquinimod, a neuroprotective therapy, Huntexil (prodopidine), SBT-20, lamotrigine (Lamictal), psychotherapy, speech therapy, physical therapy, and/or occupational therapy.

In some embodiments, an additional therapeutic agent or method is described in any of: U.S. Pat. Nos. 6,127,401; 6,169,115; 6,174,909; 6,221,904; 6,258,353; 6,300,373; 6,319,944; 6,372,736; 6,372,768; 6,395,749; 6,455,536; 6,503,899; 6,517,859; 6,525,054; 6,534,651; 6,552,041; 6,565,875; 6,630,461; 6,642,227; 6,660,748; 6,706,711; 6,746,678; 6,819,956; 6,833,478; 6,884,804; 6,921,774; 6,953,796; 7,053,057; 7,111,346; 7,132,414; 7,183,307; 7,304,061; 7,304,071; 7,404,221; 7,728,018; 7,741,365; 7,803,752; 7,807,654; 7,935,718; 8,003,610; 8,222,279; 8,278,272; 8,362,066; 8,410,110; 8,481,086; 8,604,080; 8,669,248; 8,691,824; 8,778,947; 8,802,440; 8,835,171; 8,853,198; 8,853,241; 9,005,677; 9,006,205; 9,011,937; 9,181,544; 9,193,695; 9,193,969; 9,198,944; 9,212,205; 9,216,161; 9,220,778; 9,260,394; 9,278,963; 9,289,143; 9,308,182; 9,315,532; 9,326,956; 9,351,946; 9,358,293; 9,382,314; 9,393,409; 9,415,030; 9,422,234; 9,447,006; 9,475,747; 9,504,665; 9,523,093; 9,555,071; 9,585,878; 9,604,957; 9,617,210; 9,629,815; 9,700,587; 9,796,673; 9,808,448; 9,833,621; 9,861,594; 9,861,596; 9,872,865; 9,879,063; 9,889,143; 9,913,877; 9,919,129; 9,987,286; 10,004,722; 10,087,228; 10,123,969; or 10,124,166; or any of WO/2018/227142; WO/2018/226771; WO/2018/226622; WO/2018/220457; WO/2018/218185; WO/2018/218091; WO/2018/213766; WO/2018/208636; WO/2018/206798; WO/2018/204803; WO/2018/194736; WO/2018/189393; WO/2018/187503; WO/2018/185468; WO/2018/178665; WO/2018/174839; WO/2018/174838; WO/2018/172527; WO/2018/148220; WO/2018/145009; WO/2018/138088; WO/2018/138086; WO/2018/138085; WO/2018/136635; WO/2018/132845; WO/2018/127462; WO/2018/112672; WO/2018/107072; WO/2018/093957; WO/2018/084712; WO/2018/080636; WO/2018/078042; WO/2018/076245; WO/2018/075086; WO/2018/071521; WO/2018/071508; WO/2018/071452; WO/2018/057855; WO/2018/045217; WO/2018/044808; or WO/2018/039207.

In some embodiments, a subject is administered an HTT oligonucleotide and an additional therapeutic agent, wherein the additional therapeutic agent is an agent described herein or known in the art which is useful for treatment of an HTT-related condition, disorder or disease.

In some embodiments, a second or additional therapeutic agent is administered to a subject prior, simultaneously with, or after, an HTT oligonucleotide. In some embodiments, a second or additional therapeutic agent is administered multiple times to a subject, and an HTT oligonucleotide is also administered multiple times to a subject, and the administrations are in any order.

In some embodiments, an improvement may include decreasing the expression, activity and/or level of a gene or gene product which is too high in a disease state; increasing the expression, activity and/or level of a gene or gene product which is too low in the disease state; and/or decreasing the expression, activity and/or level of a mutant and/or disease-associated variant of a gene or gene product.

In some embodiments, an HTT oligonucleotide useful for treating, ameliorating and/or preventing an HTT-related condition, disorder or disease can be administered (e.g., to a subject) via any method described herein or known in the art.

In some embodiments, provided oligonucleotides, e.g., HTT oligonucleotides are administered as pharmaceutical composition, e.g., for treating, ameliorating and/or preventing HTT-related conditions, disorders or diseases. In some embodiments, provided oligonucleotides comprise at least one chirally controlled internucleotidic linkage. In some embodiments, provided oligonucleotide compositions are chirally controlled.

In some embodiments, an additional therapeutic agent includes any one or more or all of: corticosteroid (e.g., dexamethasone); acetaminophen; H1 blocker (e.g., diphenhydramine); and/or H2 blocker (e.g., ranitidine). In some embodiments, such an additional therapeutic agent is administered to control or alleviate at least one side effect or adverse effect related to administration of an oligonucleotide.

In some cases, patients with Huntington's Disease reportedly can further suffer from an additional, associated disorder or disease or complication, such as pneumonia, heart disease, suicidal behaviors or thoughts, inability to eat, loss of weight, physical injury, e.g., from falls, etc. In some embodiments, an additional therapeutic agent is administered to treat an additional, associated disorder or disease or complication of HD.

In some cases, patients who have been administered an oligonucleotide as a medicament have experienced certain side effects or adverse effects, including: atrioventricular (AV) heart block, lower respiratory infection, constipation, teething, urinary tract infection, upper respiratory tract congestion, Ear infection, flatulence, decreased weight, thrombocytopenia, coagulation abnormalities, renal toxicity, injection site toxicity, rash, glomerulonephritis, liver toxicity, hyponatremia, macular lesions, skin lesions, pyrexia, headache, vomiting, Post-lumbar puncture syndrome, epistaxis, back pain, infection, meningitis, hydrocephalus, flushing, nausea, abdominal pain, dyspnea, hypertension, syncope, arthralgia, bronchitis, dyspepsia, dyspnea, erythema, infusion-related reaction, muscle spasms, vertigo, nasopharyngitis, upper respiratory tract infection, respiratory tract infection, pharyngitis, rhinitis, sinusitis, viral upper respiratory tract infection, upper respiratory tract congestion, arthralgia or pain (including back, neck, or musculoskeletal pain), flushing (including erythema of face or skin warm), nausea, abdominal pain, cough, chest discomfort or chest pain, headache, rash, chills, dizziness, fatigue, increased heart rate or palpitations, hypotension, hypertension, facial edema, edema, ocular adverse reactions, dry eye, blurred vision, vitreous floaters, extravasation, phlebitis, thrombophlebitis, infusion or injection site swelling, dermatitis (subcutaneous inflammation), cellulitis, erythema, injection site redness, burning sensation, injection site pain, resence of basophilic granules in Kupffer cells, poor local tolerance, increased coagulation time, complement activation, haematotoxicity, stimulation of the immune system, increased spleen weight, multiorgan lymphohistiocytic cell infiltrate, splenic extramedullary haematopoiesis, inflammatory effects, and/or reproductive toxicity.

In some embodiments, an additional therapeutic agent can be administered to the patient in order to control or alleviate one or more side effects or adverse effects associated with administration of an oligonucleotide.

In some embodiments, an oligonucleotide and one or more additional therapeutic agent are administered to a patient (in any order), wherein the additional therapeutic agent can be administered to the patient in order to control or alleviate one or more side effects or adverse effects associated with administration of the oligonucleotide.

In some embodiments, an oligonucleotide and one or more additional therapeutic agent are administered to a patient (in any order), wherein the additional therapeutic agent can be administered to the patient in order to control or alleviate one or more side effects or adverse effects associated with administration of the oligonucleotide, and wherein the oligonucleotide targets any target, including but not limited to: HTT, DMD, APOC3, PNPLA3, C9orf72, or SMN2, or any other gene target.

In some embodiments, an oligonucleotide and one or more additional therapeutic agent are administered to a patient (in any order), wherein the additional therapeutic agent can be administered to the patient in order to control or alleviate one or more side effects or adverse effects associated with administration of the oligonucleotide, and wherein the oligonucleotide operates via any biochemical mechanism, including but not limited to: decreasing the level, expression and/or activity of a target gene or a gene product thereof, increasing or decreasing skipping of one or more exons in a target gene mRNA, a RNaseH-mediated mechanism, a steric hindrance-mediated mechanism, and/or a RNA interference-mediated mechanism, wherein the oligonucleotide is single- or double-stranded.

In some embodiments, an oligonucleotide and one or more additional therapeutic agent are administered to a patient (in any order), wherein the additional therapeutic agent can be administered to the patient in order to control or alleviate one or more side effects or adverse effects associated with administration of the oligonucleotide, and wherein the oligonucleotide operates via any biochemical mechanism, including but not limited to: decreasing the level, expression and/or activity of a target gene or a gene product thereof, increasing or decreasing skipping of one or more exons in a target gene mRNA, a RNaseH-mediated mechanism, a steric hindrance-mediated mechanism, and/or a RNA interference-mediated mechanism, wherein the oligonucleotide is single- or double-stranded, and wherein the oligonucleotide targets any target, including but not limited to: HTT, DMD, APOC3, PNPLA3, C9orf72, or SMN2, or any other gene target.

In some embodiments, an oligonucleotide composition and one or more additional therapeutic agent are administered to a patient (in any order), wherein the additional therapeutic agent can be administered to the patient in order to control or alleviate one or more side effects or adverse effects associated with administration of the oligonucleotide composition, and wherein the oligonucleotide composition is chirally controlled or comprises at least one chirally controlled internucleotidic linkage (including but not limited to a chirally controlled phosphorothioate).

### Administration of Oligonucleotides and Compositions Thereof

Many delivery methods, regimen, etc. can be utilized in accordance with the present disclosure for administering provided oligonucleotides and compositions thereof (typically pharmaceutical compositions for therapeutic purposes), including various technologies known in the art.

In some embodiments, an oligonucleotide composition, e.g., an HTT oligonucleotide composition, is administered at a dose and/or frequency lower than that of an otherwise comparable reference oligonucleotide composition and has comparable or improved effects. In some embodiments, a chirally controlled oligonucleotide composition is administered at a dose and/or frequency lower than that of a comparable, otherwise identical stereorandom reference oligonucleotide composition and with comparable or improved effects, e.g., in improving the knockdown of the target transcript.

In some embodiments, the present disclosure recognizes that properties and activities, e.g., knockdown activity, stability, toxicity, etc. of oligonucleotides and compositions thereof can be modulated and optimized by chemical modifications and/or stereochemistry. In some embodiments, the present disclosure provides methods for optimizing oligonucleotide properties and/or activities through chemical modifications and/or stereochemistry. In some embodiments, the present disclosure provides oligonucleotides and compositions thereof with improved properties and/or activities. Without wishing to be bound by any theory, due to, e.g., their better activity, stability, delivery, distribution, toxicity, pharmacokinetic, pharmacodynamics and/or efficacy profiles, Applicant notes that provided oligonucleotides and compositions thereof in some embodiments can be administered at lower dosage and/or reduced frequency to achieve comparable or better efficacy, and in some embodiments can be administered at higher dosage and/or increased frequency to provide enhanced effects.

In some embodiments, the present disclosure provides, in a method of administering a oligonucleotide composition comprising a plurality of oligonucleotides sharing a common base sequence, the improvement comprising administering an oligonucleotide comprising a plurality of oligonucleotides that is characterized by improved delivery relative to a reference oligonucleotide composition of the same common base sequence.

In some embodiments, provided oligonucleotides, compositions and methods provide improved delivery. In some embodiments, provided oligonucleotides, compositions and methods provide improved cytoplasmatic delivery. In some embodiments, improved delivery is to a population of cells. In some embodiments, improved delivery is to a tissue. In some embodiments, improved delivery is to an organ. In some embodiments, improved delivery is to an organism, e.g., a patient or subject. Example structural elements (e.g., chemical modifications, stereochemistry, combinations thereof, etc.), oligonucleotides, compositions and methods that provide improved delivery are extensively described in the present disclosure.

Various dosing regimens can be utilized to administer oligonucleotides and compositions fo the present disclosure. In some embodiments, multiple unit doses are administered, separated by periods of time. In some embodiments, a given composition has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second (or subsequent) dose amount that is the same as or different from the first dose (or another prior dose) amount. In some embodiments, a chirally controlled oligonucleotide composition is administered according to a dosing regimen that differs from that utilized for a non-chirally controlled (e.g., stereorandom) oligonucleotide composition of the same sequence, and/or of a different chirally controlled oligonucleotide composition of the same sequence. In some embodiments, a chirally controlled oligonucleotide composition is administered according to a dosing regimen that is reduced as compared with that of a chirally uncontrolled (e.g., stereorandom) oligonucleotide composition of the same sequence in that it achieves a lower level of total exposure over a given unit of time, involves one or more lower unit doses, and/or includes a smaller number of doses over a given unit of time. In some embodiments, a chirally uncontrolled oligonucleotide is administered according to a dosing regimen that extends for a longer period of time than does that of a chirally uncontrolled (e.g., stereorandom) oligonucleotide composition of the same sequence Without wishing to be limited by theory, Applicant notes that in some embodiments, the shorter dosing regimen, and/or longer time periods between doses, may be due to the improved stability, bioavailability, and/or efficacy of a chirally controlled oligonucleotide composition. In some embodiments, with their improved delivery (and other properties), provided compositions can be administered in lower dosages and/or with lower frequency to achieve biological effects, for example, clinical efficacy.

### Pharmaceutical Compositions

In some embodiments, the present disclosure provides pharmaceutical compositions comprising a provided compound, e.g., an oligonucleotide, or a pharmaceutically acceptable salt thereof, and a pharmaceutical carrier. In some embodiments, for therapeutic and clinical purposes, oligonucleotides of the present disclosure are provided as pharmaceutical compositions. As appreciated by those skilled in the art, oligonucleotides of the present disclosure can be provided in their acid, base or salt forms. In some embodiments, oligonucleotides can be in acid forms, e.g., for natural phosphate linkages, in the form of —OP(O)(OH)O—; for phosphorothioate internucleotidic linkages, in the form of —OP(O)(SH)O—; etc. In some embodiments, provided oligonucleotides can be in salt forms, e.g., for natural phosphate linkages, in the form of —OP(O)(ONa)O— in sodium salts; for phosphorothioate internucleotidic linkages, in the form of —OP(O)(SNa)O— in sodium salts; etc. Unless otherwise noted, oligonucleotides of the present disclosure can exist in acid, base and/or salt forms.

When used as therapeutics, an HTT oligonucleotide or oligonucleotide composition thereof is typically administered as a pharmaceutical composition. In some embodiments, a pharmaceutical composition is suitable for administration of an oligonucleotide to an area of a body affected by a condition, disorder or disease. In some embodiments, a pharmaceutical composition comprises a therapeutically effective amount of a provided oligonucleotide or a pharmaceutically acceptable salt thereof, and a pharmaceutically acceptable inactive ingredient. In some embodiments, a pharmaceutically acceptable inactive ingredient is selected from pharmaceutically acceptable diluents, pharmaceutically acceptable excipients, and pharmaceutically acceptable carriers. In some embodiments, a pharmaceutically acceptable inactive ingredient is a pharmaceutically acceptable carrier.

In some embodiments, a provided oligonucleotide is formulated for administration to and/or contact with a body cell and/or tissue expressing its target. For example, in some embodiments, a provided HTT oligonucleotide is formulated for administration to a body cell and/or tissue expressing HTT. In some embodiments, such a body cell and/or tissue are a neuron or a cell and/or tissue of the central nervous system. In some embodiments, broad distribution of oligonucleotides and compositions may be achieved with intraparenchymal administration, intrathecal administration, or intracerebroventricular administration.

In some embodiments, the pharmaceutical composition is formulated for intravenous injection, oral administration, buccal administration, inhalation, nasal administration, topical administration, ophthalmic administration or otic administration. In some embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

In some embodiments, the present disclosure provides a pharmaceutical composition comprising chirally controlled oligonucleotide or composition thereof, in admixture with a pharmaceutically acceptable inactive ingredient (e.g., a pharmaceutically acceptable excipient, a pharmaceutically acceptable carrier, etc.). One of skill in the art will recognize that the pharmaceutical compositions include pharmaceutically acceptable salts of provided oligonucleotide or compositions. In some embodiments, a pharmaceutical composition is a chirally controlled oligonucleotide composition. In some embodiments, a pharmaceutical composition is a stereopure oligonucleotide composition.

In some embodiments, the present disclosure provides salts of oligonucleotides and pharmaceutical compositions thereof. In some embodiments, a salt is a pharmaceutically acceptable salt. In some embodiments, a pharmaceutical composition comprises an oligonucleotide, optionally in its salt form, and a sodium salt. In some embodiments, a pharmaceutical composition comprises an oligonucleotide, optionally in its salt form, and sodium chloride. In some embodiments, each hydrogen ion of an oligonucleotide that may be donated to a base (e.g., under conditions of an aqueous solution, a pharmaceutical composition, etc.) is replaced by a non-H⁺ cation. For example, in some embodiments, a pharmaceutically acceptable salt of an oligonucleotide is an all-metal ion salt, wherein each hydrogen ion (for example, of —OH, —SH, etc.) of each internucleotidic linkage (e.g., a natural phosphate linkage, a phosphorothioate internucleotidic linkage, etc.) is replaced by a metal ion. Various suitable metal salts for pharmaceutical compositions are widely known in the art and can be utilized in accordance with the present disclosure. In some embodiments, a pharmaceutically acceptable salt is a sodium salt. In some embodiments, a pharmaceutically acceptable salt is magnesium salt. In some embodiments, a pharmaceutically acceptable salt is a calcium salt. In some embodiments, a pharmaceutically acceptable salt is a potassium salt. In some embodiments, a pharmaceutically acceptable salt is an ammonium salt (cation N(R)₄⁺). In some embodiments, a pharmaceutically acceptable salt comprises one and no more than one types of cation. In some embodiments, a pharmaceutically acceptable salt comprises two or more types of cation. In some embodiments, a cation is Li⁺, Na⁺, K⁺, Mg²⁺ or Ca²⁺. In some embodiments, a pharmaceutically acceptable salt is an all-sodium salt. In some embodiments, a pharmaceutically acceptable salt is an all-sodium salt, wherein each internucleotidic linkage which is a natural phosphate linkage (acid form —O—P(O)(OH)—O—), if any, exists as its sodium salt form (—O—P(O)(ONa)—O—), and each internucleotidic linkage which is a phosphorothioate internucleotidic linkage linkage (acid form —O—P(O)(SH)—O—), if any, exists as its sodium salt form (—O—P(O)(SNa)—O—).

Various technologies for delivering nucleic acids and/or oligonucleotides are known in the art can be utilized in accordance with the present disclosure. For example, a variety of supramolecular nanocarriers can be used to deliver nucleic acids. Example nanocarriers include, but are not limited to liposomes, cationic polymer complexes and various polymeric compounds. Complexation of nucleic acids with various polycations is another approach for intracellular delivery; this includes use of PEGylated polycations, polyethyleneamine (PEI) complexes, cationic block co-polymers, and dendrimers. Several cationic nanocarriers, including PEI and polyamidoamine dendrimers help to release contents from endosomes. Other approaches include use of polymeric nanoparticles, microspheres, liposomes, dendrimers, biodegradable polymers, conjugates, prodrugs, inorganic colloids such as sulfur or iron, antibodies, implants, biodegradable implants, biodegradable microspheres, osmotically controlled implants, lipid nanoparticles, emulsions, oily solutions, aqueous solutions, biodegradable polymers, poly(lactide-coglycolic acid), poly(lactic acid), liquid depot, polymer micelles, quantum dots and lipoplexes. In some embodiments, an oligonucleotide is conjugated to another molecule.

In therapeutic and/or diagnostic applications, compounds, e.g., oligonucleotides, of the disclosure can be formulated for a variety of modes of administration, including systemic and topical or localized administration. Techniques and formulations generally may be found in Remington, The Science and Practice of Pharmacy (20th ed. 2000).

Pharmaceutically acceptable salts for basic moieties are generally well known to those of ordinary skill in the art, and may include, e.g., acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington, The Science and Practice of Pharmacy (20th ed. 2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

In some embodiments, provided oligonucleotides are formulated in pharmaceutical compositions described in WO 2005/060697, WO 2011/076807 or WO 2014/136086.

Depending on the specific conditions, disorders or diseases being treated, provided agents, e.g., oligonucleotides, may be formulated into liquid or solid dosage forms and administered systemically or locally. Provided oligonucleotides may be delivered, for example, in a timed- or sustained-low release form as is known to those skilled in the art. Techniques for formulation and administration may be found in Remington, The Science and Practice of Pharmacy (20th ed. 2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or another mode of delivery.

For injection, provided agents, e.g., oligonucleotides may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulations. Such penetrants are generally known in the art and can be utilized in accordance with the present disclosure.

Use of pharmaceutically acceptable carriers to formulate compounds, e.g., provided oligonucleotides, for the practice of the disclosure into dosages suitable for various mods of administration is well known in the art. With proper choice of carrier and suitable manufacturing practice, compositions of the present disclosure, e.g., those formulated as solutions, may be administered via various routes, e.g., parenterally, such as by intravenous injection.

In some embodiments, a composition comprising an oligonucleotide, e.g., an HTT oligonucleotide, further comprises any or all of: calcium chloride dihydrate, magnesium chloride hexahydrate, potassium chloride, sodium chloride, sodium phosphate dibasic anhydrous, sodium phosphate, monobasic dihydrate, and/or water for Injection. In some embodiments, a composition further comprises any or all of: calcium chloride dihydrate (0.21 mg) USP, magnesium chloride hexahydrate (0.16 mg) USP, potassium chloride (0.22 mg) USP, sodium chloride (8.77 mg) USP, sodium phosphate dibasic anhydrous (0.10 mg) USP, sodium phosphate monobasic dihydrate (0.05 m g) USP, and Water for Injection USP.

In some embodiments, a composition comprising an oligonucleotide further comprises any or all of: cholesterol, (6Z,9Z,28Z,31Z)-heptatriaconta-6,9,28,31-tetraen-19-yl-4-(dimethylamino) butanoate(DLin-MC3-DMA), 1,2-distearoyl-sn-glycero-3-phosphocholine (DSPC), alpha-(3′-{[1,2-di(myristyloxy)propanoxy] carbonylamino}propyl)-omega-methoxy, polyoxyethylene(PEG2000-C-DMG), potassium phosphate monobasic anhydrous NF, sodium chloride, sodium phosphate dibasic heptahydrate, and Water for Injection. In some embodiments, the pH of a composition comprising an oligonucleotide, e.g., an HTT oligonucleotide, is ˜7.0. In some embodiments, a composition comprising an oligonucleotide further comprises any or all of: 6.2 mg cholesterol USP, 13.0 mg (6Z,9Z,28Z,31Z)-heptatriaconta-6,9,28,31-tetraen-19-yl-4-(dimethylamino) butanoate(DLin-MC3-DMA), 3.3 mg 1,2-distearoyl-sn-glycero-3-phosphocholine (DSPC), 1.6 mg α-(3′-{[1,2-di(myristyloxy)propanoxy]carbonylamino}propyl)-ω-methoxy, polyoxyethylene(PEG2000-C-DMG), 0.2 mg potassium phosphate monobasic anhydrous NF, 8.8 mg sodium chloride USP, 2.3 mg sodium phosphate dibasic heptahydrate USP, and Water for Injection USP, in an approximately 1 mL total volume.

Provided compounds, e.g., oligonucleotides, can be formulated readily using pharmaceutically acceptable carriers well known in the art into dosages suitable for oral administration. In some embodiments, such carriers enable provided oligonucleotides to be formulated as tablets, pills, capsules, liquids, gels, syrups, slurries, suspensions and the like, for, e.g., oral ingestion by a subject (e.g., patient) to be treated.

For nasal or inhalation delivery, provided compounds, e.g., oligonucleotides, may be formulated by methods known to those of skill in the art, and may include, e.g., examples of solubilizing, diluting, or dispersing substances such as saline, preservatives, such as benzyl alcohol, absorption promoters, and fluorocarbons.

In certain embodiments, oligonucleotides and compositions are delivered to the CNS. In certain embodiments, oligonucleotides and compositions are delivered to the cerebrospinal fluid. In certain embodiments, oligonucleotides and compositions are administered to the brain parenchyma. In certain embodiments, oligonucleotides and compositions are delivered to an animal/subject by intrathecal administration, or intracerebroventricular administration. Broad distribution of oligonucleotides and compositions may be achieved with methods of administration described herein and/or known in the art.

In certain embodiments, parenteral administration is by injection, by, e.g., a syringe, a pump, etc. In certain embodiments, an injection is a bolus injection. In certain embodiments, an injection is administered directly to a tissue or location, such as striatum, caudate, cortex, hippocampus and/or cerebellum.

In certain embodiments, methods of specifically localizing provided compounds, e.g., oligonucleotides, such as by bolus injection, may decrease median effective concentration (EC50) by a factor of 20, 25, 30, 35, 40, 45 or 50. In certain embodiments, a targeted tissue is brain tissue. In certain embodiments, a targeted tissue is striatal tissue. In certain embodiments, decreasing EC50 is desirable because it reduces the dose required to achieve a pharmacological result in a patient in need thereof.

In certain embodiments, a provided oligonucleotide is delivered by injection or infusion once every month, every two months, every 90 days, every 3 months, every 6 months, twice a year or once a year.

Pharmaceutical compositions suitable for use in the present disclosure include compositions wherein the active ingredients, e.g., oligonucleotides, are contained in effective amounts to achieve their intended purposes. Determination of the effective amounts is well within the capability of those skilled in the art, especially in light of the detailed disclosure provided herein.

In addition to active ingredients, pharmaceutical compositions may contain suitable pharmaceutically acceptable carriers comprising excipients and auxiliaries which facilitate processing of an active compound into preparations which can be used pharmaceutically. Preparations formulated for oral administration may be in the form of tablets, dragees, capsules, or solutions.

In some embodiments, pharmaceutical compositions for oral use can be obtained by combining an active compound with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

In some embodiments, dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. Push-fit capsules can contain active ingredients, e.g., oligonucleotides, in admixture with fillers such as lactose, binders such as starches, and/or lubricants such as talc or magnesium stearate and, optionally, stabilizers. In soft capsules, active compounds, e.g., oligonucleotides, may be dissolved or suspended in suitable liquids, such as fatty oils, liquid paraffin, or liquid polyethylene glycols (PEGs). In addition, stabilizers may be added.

In some embodiments, a provided composition comprises a lipid. In some embodiments, a lipid is conjugated to an active compound, e.g., an oligonucleotide. In some embodiments, a lipid is not conjugated to an active compound. In some embodiments, a lipid comprises a C₁₀-C₄₀ linear, saturated or partially unsaturated, aliphatic chain. In some embodiments, a lipid comprises a C₁₀-C₄₀ linear, saturated or partially unsaturated, aliphatic chain, optionally substituted with one or more C₁₋₄ aliphatic group. In some embodiments, the lipid is selected from the group consisting of lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl alcohol. In some embodiments, an active compound is a provided oligonucleotide. In some embodiments, a composition comprises a lipid and an active compound, and further comprises another component which is another lipid or a targeting compound or moiety. In some embodiments, a lipid is an amino lipid; an amphipathic lipid; an anionic lipid; an apolipoprotein; a cationic lipid; a low molecular weight cationic lipid; a cationic lipid such as CLinDMA and DLinDMA; an ionizable cationic lipid; a cloaking component; a helper lipid; a lipopeptide; a neutral lipid; a neutral zwitterionic lipid; a hydrophobic small molecule; a hydrophobic vitamin; a PEG-lipid; an uncharged lipid modified with one or more hydrophilic polymers; phospholipid; a phospholipid such as 1,2-dioleoyl-sn-glycero-3-phosphoethanolamine; a stealth lipid; a sterol; a cholesterol; a targeting lipid; or another lipid described herein or reported in the art suitable for pharmaceutical uses. In some embodiments, a composition comprises a lipid and a portion of another lipid capable of mediating at least one function of another lipid. In some embodiments, a targeting compound or moiety is capable of targeting a compound (e.g., an oligonucleotide) to a particular cell or tissue or subset of cells or tissues. In some embodiments, a targeting moiety is designed to take advantage of cell- or tissue-specific expression of particular targets, receptors, proteins, or another subcellular component. In some embodiments, a targeting moiety is a ligand (e.g., a small molecule, antibody, peptide, protein, carbohydrate, aptamer, etc.) that targets a composition to a cell or tissue, and/or binds to a target, receptor, protein, or another subcellular component.

Certain example lipids for delivery of an active compound, e.g., an oligonucleotide, allow (e.g., do not prevent or interfere with) the function of an active compound. In some embodiments, a lipid is lauric acid, myristic acid, palmitic acid, stearic acid, oleic acid, linoleic acid, alpha-linolenic acid, gamma-linolenic acid, docosahexaenoic acid (cis-DHA), turbinaric acid or dilinoleyl alcohol.

As described in the present disclosure, lipid conjugation, such as conjugation with fatty acids, may improve one or more properties of oligonucleotides.

In some embodiments, a composition for delivery of an active compound, e.g., an oligonucleotide, is capable of targeting an active compound to particular cells or tissues as desired. In some embodiments, a composition for delivery of an active compound is capable of targeting an active compound to a muscle cell or tissue. In some embodiments, the present disclosure provides compositions and methods related to delivery of active compounds, wherein the compositions comprise an active compound and a lipid. In various embodiments to a muscle cell or tissue, a lipid is selected from lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl alcohol.

In some embodiments, a composition comprising an oligonucleotide is lyophilized. In some embodiments, a composition comprising an oligonucleotide is lyophilized, and the lyophilized oligonucleotide is in a vial. In some embodiments, the vial is back filled with nitrogen. In some embodiments, the lyophilized oligonucleotide composition is reconstituted prior to administration. In some embodiments, the lyophilized oligonucleotide composition is reconstituted with a sodium chloride solution prior to administration. In some embodiments, the lyophilized oligonucleotide composition is reconstituted with a 0.9% sodium chloride solution prior to administration. In some embodiments, reconstitution occurs at the clinical site for administration. In some embodiments, in a lyophilized composition, an oligonucleotide composition is chirally controlled or comprises at least one chirally controlled internucleotidic linkage and/or the oligonucleotide targets any target, including but not limited to: HTT, DMD, APOC3, PNPLA3, C9orf72, or SMN2, or any other gene target.

### Certain Embodiments of Variables

In some embodiments, the present disclosure uses variables in formulae, patterns, etc. Certain example embodiments of such variables are described below. As appreciated by those skilled in the art, embodiments for each variable described below or elsewhere in the present disclosure may be independently and optionally combined with embodiments of other variables in the same formulae, patterns, etc., described below or elsewhere in the present disclosure.

In some embodiments, R^{5s}-L^{s}- is —CH₂OH. In some embodiments, R^{5s}-L^{s}- is —C(R^{5s})₂OH, wherein R^{5s} is as described in the present disclosure. In some embodiments, R^{5s}-L^{s}- is —CH(R^{5s})—OH, wherein R^{5s} is as described in the present disclosure.

In some embodiments, BA is an optionally substituted group selected from C₃₋₃₀ cycloaliphatic, C₆₋₃₀ aryl, C₅₋₃₀ heteroaryl having 1-10 heteroatoms independently selected from oxygen, nitrogen, sulfur, phosphorus and silicon, C₃₋₃₀ heterocyclyl having 1-10 heteroatoms independently selected from oxygen, nitrogen, sulfur, phosphorus and silicon, a natural nucleobase moiety, and a modified nucleobase moiety. In some embodiments, BA is an optionally substituted group selected from C₅₋₃₀ heteroaryl having 1-10 heteroatoms independently selected from oxygen, nitrogen, sulfur, phosphorus and silicon, C₃₋₃₀ heterocyclyl having 1-10 heteroatoms independently selected from oxygen, nitrogen, sulfur, phosphorus and silicon, a natural nucleobase moiety, and a modified nucleobase moiety. In some embodiments, BA is an optionally substituted group selected from C₅₋₃₀ heteroaryl having 1-10 heteroatoms independently selected from oxygen, nitrogen, sulfur, phosphorus and silicon, a natural nucleobase moiety, and a modified nucleobase moiety. In some embodiments, BA is optionally substituted C₅₋₃₀ heteroaryl having 1-10 heteroatoms independently selected from oxygen, nitrogen, and sulfur. In some embodiments, BA is optionally substituted natural nucleobases and tautomers thereof. In some embodiments, BA is protected natural nucleobases and tautomers thereof. Various nucleobase protecting groups for oligonucleotide synthesis are known and can be utilized in accordance with the present disclosure. In some embodiments, BA is an optionally substituted nucleobase selected from adenine, cytosine, guanosine, thymine, and uracil, and tautomers thereof. In some embodiments, BA is an optionally protected nucleobase selected from adenine, cytosine, guanosine, thymine, and uracil, and tautomers thereof.

In some embodiments, BA is optionally substituted C₃₋₃₀ cycloaliphatic. In some embodiments, BA is optionally substituted C₆₋₃₀ aryl. In some embodiments, BA is optionally substituted C₃₋₃₀ heterocyclyl. In some embodiments, BA is optionally substituted C₅₋₃₀ heteroaryl. In some embodiments, BA is an optionally substituted natural base moiety. In some embodiments, BA is an optionally substituted modified base moiety. BA is an optionally substituted group selected from C₃₋₃₀ cycloaliphatic, C₆₋₃₀ aryl, C₃₋₃₀ heterocyclyl, and C₅₋₃₀ heteroaryl. In some embodiments, BA is an optionally substituted group selected from C₃₋₃₀ cycloaliphatic, C₆₋₃₀ aryl, C₃₋₃₀ heterocyclyl, C₅₋₃₀ heteroaryl, and a natural nucleobase moiety.

In some embodiments, BA is connected through an aromatic ring. In some embodiments, BA is connected through a heteroatom. In some embodiments, BA is connected through a ring heteroatom of an aromatic ring. In some embodiments, BA is connected through a ring nitrogen atom of an aromatic ring.

In some embodiments, BA is a natural nucleobase. In some embodiments, BA is an optionally substituted natural nucleobase. In some embodiments, BA is a substituted natural nucleobase. In some embodiments, BA is optionally substituted, or an optionally substituted tautomer of, A, T, C, U, or G. In some embodiments, BA is natural nucleobase A, T, C, U, or G. In some embodiments, BA is an optionally substituted group selected from natural nucleobases A, T, C, U, and G.

In some embodiments, BA is an optionally substituted purine base residue. In some embodiments, BA is a protected purine base residue. In some embodiments, BA is an optionally substituted adenine residue. In some embodiments, BA is a protected adenine residue. In some embodiments, BA is an optionally substituted guanine residue. In some embodiments, BA is a protected guanine residue. In some embodiments, BA is an optionally substituted cytosine residue. In some embodiments, BA is a protected cytosine residue. In some embodiments, BA is an optionally substituted thymine residue. In some embodiments, BA is a protected thymine residue. In some embodiments, BA is an optionally substituted uracil residue. In some embodiments, BA is a protected uracil residue. In some embodiments, BA is an optionally substituted 5-methylcytosine residue. In some embodiments, BA is a protected 5-methylcytosine residue.

In some embodiments, BA is a protected base residue as used in oligonucleotide preparation. In some embodiments, BA is a nucleobase as described in the present disclosure.

In some embodiments, each R^{s} is independently —H, halogen, —CN, —N₃, —NO, —NO₂, -L^{s}-R′, -L^{s}-Si(R)₃, -L^{s}-OR′, -L^{s}-SR′, -L^{s}-N(R′)₂, —O-L^{s}-R′, —O-L^{s}-Si(R)₃, —O-L^{s}-OR′, —O-L^{s}-SR′, or —O-L^{s}-N(R′)₂ as described in the present disclosure.

In some embodiments, R^{s} is R′, wherein R is as described in the present disclosure. In some embodiments, R^{s} is R, wherein R is as described in the present disclosure. In some embodiments, R^{s} is optionally substituted C₁₋₃₀ heteroaliphatic. In some embodiments, R^{s} comprises one or more silicon atoms. In some embodiments, R^{s} is —CH₂Si(Ph)₂CH₃.

In some embodiments, R^{s} is -L^{s}-R′. In some embodiments, R^{s} is -L^{s}-R′ wherein -L^{s}- is a bivalent, optionally substituted C₁₋₃₀ heteroaliphatic group. In some embodiments, R^{s} is —CH₂Si(Ph)₂CH₃.

In some embodiments, R^{s} is —F. In some embodiments, R^{s} is —Cl. In some embodiments, R^{s} is —Br. In some embodiments, R^{s} is —I. In some embodiments, R^{s} is —CN. In some embodiments, R^{s} is —N₃. In some embodiments, R^{s} is —NO. In some embodiments, R^{s} is —NO₂. In some embodiments, R^{s} is -L^{s}-Si(R)₃. In some embodiments, R^{s} is —Si(R)₃. In some embodiments, R^{s} is -L^{s}-R′. In some embodiments, R^{s} is —R′. In some embodiments, R^{s} is -L^{s}-OR′. In some embodiments, R^{s} is —OR′. In some embodiments, R^{s} is -L^{s}-SR′. In some embodiments, R^{s} is —SR′. In some embodiments, R^{s} is -L^{s}-N(R′)₂. In some embodiments, R^{s} is —N(R′)₂. In some embodiments, R^{s} is —O-L^{s}-R′. In some embodiments, R^{s} is —O-L^{s}-Si(R)₃. In some embodiments, R^{s} is —O-L^{s}-OR′. In some embodiments, R^{s} is —O-L^{s}-SR′. In some embodiments, R^{s} is —O-L^{s}-N(R′)₂. In some embodiments, R^{s} is a 2′-modification as described in the present disclosure. In some embodiments, R^{s} is —OR, wherein R is as described in the present disclosure. In some embodiments, R^{s} is —OR, wherein R is optionally substituted C₁₋₆ aliphatic. In some embodiments, R^{s} is —OMe. In some embodiments, R^{s} is —OCH₂CH₂OMe.

In some embodiments, s is 0-20. In some embodiments, s is 1-20. In some embodiments, s is 1-5. In some embodiments, s is 1. In some embodiments, s is 2. In some embodiments, s is 3. In some embodiments, s is 4. In some embodiments, s is 5. In some embodiments, s is 6. In some embodiments, s is 7. In some embodiments, s is 8. In some embodiments, s is 9. In some embodiments, s is 10. In some embodiments, s is 11. In some embodiments, s is 12. In some embodiments, s is 13. In some embodiments, s is 14. In some embodiments, s is 15. In some embodiments, s is 16. In some embodiments, s is 17. In some embodiments, s is 18. In some embodiments, s is 19. In some embodiments, s is 20.

In some embodiments, L^{s} is L, wherein L is as described in the present disclosure. In some embodiments, L is a bivalent optionally substituted methylene group. In some embodiments, L^{s} is —CH₂—. In some embodiments, L^{s} is —C(R′)₂—. In some embodiments, L^{s} is —CH(R′)—. In some embodiments, L^{s} is —CHR—. In some embodiments, each L^{s} is independently a covalent bond, or a bivalent, optionally substituted, linear or branched group selected from a C₁₋₃₀ aliphatic group and a C₁₋₃₀ heteroaliphatic group having 1-10 heteroatoms independently selected from oxygen, nitrogen, sulfur, phosphorus and silicon, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms independently selected from oxygen, nitrogen, sulfur, phosphorus and silicon, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more carbon atoms are optionally and independently replaced with Cy^{L}.

In some embodiments, L^{s} is a covalent bond, or a bivalent, optionally substituted, linear or branched group selected from a C₁₋₃₀ aliphatic group and a C₁₋₃₀ heteroaliphatic group having 1-10 heteroatoms independently selected from oxygen, nitrogen, sulfur, phosphorus and silicon, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms independently selected from oxygen, nitrogen, sulfur, phosphorus and silicon, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more carbon atoms are optionally and independently replaced with Cy^{L}. In some embodiments, L^{s} is a covalent bond, or a bivalent, optionally substituted, linear or branched C₁₋₃₀ aliphatic group, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms independently selected from oxygen, nitrogen, sulfur, phosphorus and silicon, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more carbon atoms are optionally and independently replaced with Cy^{L}. In some embodiments, L^{s} is a covalent bond, or a bivalent, optionally substituted, linear or branched C₁₋₃₀ heteroaliphatic group having 1-10 heteroatoms independently selected from oxygen, nitrogen, sulfur, phosphorus and silicon, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms independently selected from oxygen, nitrogen, sulfur, phosphorus and silicon, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more carbon atoms are optionally and independently replaced with Cy^{L}. In some embodiments, L^{s} is a covalent bond, or a bivalent, optionally substituted, linear or branched group selected from a C₁₋₃₀ aliphatic group and a C₁₋₃₀ heteroaliphatic group having 1-10 heteroatoms independently selected from oxygen, nitrogen, sulfur, phosphorus and silicon, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms independently selected from oxygen, nitrogen, sulfur, phosphorus and silicon, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, and one or more carbon atoms are optionally and independently replaced with Cy^{L}. In some embodiments, L^{s} is a covalent bond, or a bivalent, optionally substituted, linear or branched group selected from a C₁₋₁₀ aliphatic group and a C₁₋₁₀ heteroaliphatic group having 1-5 heteroatoms independently selected from oxygen, nitrogen, sulfur, phosphorus and silicon, wherein one or more methylene units are optionally and independently replaced by an optionally substituted group selected from C₁₋₆ alkylene, C₁₋₆ alkenylene, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, and —C(O)O—, and one or more carbon atoms are optionally and independently replaced with Cy^{L}. In some embodiments, L^{s} is a covalent bond, or a bivalent, optionally substituted, linear or branched group selected from a C₁₋₁₀ aliphatic group and a C₁₋₁₀ heteroaliphatic group having 1-5 heteroatoms independently selected from oxygen, nitrogen, sulfur, phosphorus and silicon, wherein one or more methylene units are optionally and independently replaced by an optionally substituted group selected from —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, and —C(O)O—.

In some embodiments, L^{s} is a covalent bond. In some embodiments, L^{s} is optionally substituted bivalent C₁₋₃₀ aliphatic. In some embodiments, L^{s} is optionally substituted bivalent C₁₋₃₀ heteroaliphatic having 1-10 heteroatoms independently selected from boron, oxygen, nitrogen, sulfur, phosphorus and silicon.

In some embodiments, aliphatic moieties, e.g. those of L^{s}, R, etc., either monovalent or bivalent or multivalent, and can contain any number of carbon atoms (before any optional substitution) within its range, e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, etc. In some embodiments, heteroaliphatic moieties, e.g. those of L^{s}, R, etc., either monovalent or bivalent or multivalent, and can contain any number of carbon atoms (before any optional substitution) within its range, e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, etc.

In some embodiments, a methylene unit is replaced with -Cy-, wherein -Cy- is as described in the present disclosure. In some embodiments, one or more methylene unit is optionally and independently substituted with —O—, —S—, —N(R′)—, —C(O)—, —S(O)—, —S(O)₂—, —P(O)(OR′)—, —P(O)(SR′)—, —P(S)(OR′)—, or —P(S)(OR′)—. In some embodiments, a methylene unit is replaced with —O—. In some embodiments, a methylene unit is replaced with —S—. In some embodiments, a methylene unit is replaced with —N(R′)—. In some embodiments, a methylene unit is replaced with —C(O)—. In some embodiments, a methylene unit is replaced with —S(O)—. In some embodiments, a methylene unit is replaced with —S(O)₂—. In some embodiments, a methylene unit is replaced with —P(O)(OR′)—. In some embodiments, a methylene unit is replaced with —P(O)(SR′)—. In some embodiments, a methylene unit is replaced with —P(O)(R′)—. In some embodiments, a methylene unit is replaced with —P(O)(NR′)—. In some embodiments, a methylene unit is replaced with —P(S)(OR′)—. In some embodiments, a methylene unit is replaced with —P(S)(SR′)—. In some embodiments, a methylene unit is replaced with —P(S)(R′)—. In some embodiments, a methylene unit is replaced with —P(S)(NR′)—. In some embodiments, a methylene unit is replaced with —P(R′)—. In some embodiments, a methylene unit is replaced with —P(OR′)—. In some embodiments, a methylene unit is replaced with —P(SR′)—. In some embodiments, a methylene unit is replaced with —P(NR′)—. In some embodiments, a methylene unit is replaced with —P(OR′)[B(R′)₃]—. In some embodiments, one or more methylene unit is optionally and independently substituted with —O—, —S—, —N(R′)—, —C(O)—, —S(O)—, —S(O)₂—, —P(O)(OR′)—, —P(O)(SR′)—, —P(S)(OR′)—, or —P(S)(OR′)—. In some embodiments, a methylene unit is replaced with —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, each of which may independently be an internucleotidic linkage.

In some embodiments, L^{s}, e.g., when connected to R^{s}, is —CH₂—. In some embodiments, L^{s} is —C(R)₂—, wherein at least one R is not hydrogen. In some embodiments, L^{s} is —CHR—. In some embodiments, R is hydrogen. In some embodiments, L^{s} is —CHR—, wherein R is not hydrogen. In some embodiments, C of —CHR— is chiral. In some embodiments, L^{s} is —(R)—CHR—, wherein C of —CHR— is chiral. In some embodiments, L^{s} is —(S)—CHR—, wherein C of —CHR— is chiral. In some embodiments, R is optionally substituted C₁₋₆ aliphatic. In some embodiments, R is optionally substituted C₁₋₆ alkyl. In some embodiments, R is optionally substituted C₁₋₅ aliphatic. In some embodiments, R is optionally substituted C₁₋₅ alkyl. In some embodiments, R is optionally substituted C₁₋₄ aliphatic. In some embodiments, R is optionally substituted C₁₋₄ alkyl. In some embodiments, R is optionally substituted C₁₋₃ aliphatic. In some embodiments, R is optionally substituted C₁₋₃ alkyl. In some embodiments, R is optionally substituted C₂ aliphatic. In some embodiments, R is optionally substituted methyl. In some embodiments, R is C₁₋₆ aliphatic. In some embodiments, R is C₁₋₆ alkyl. In some embodiments, R is C₁₋₅ aliphatic. In some embodiments, R is C₁₋₅ alkyl. In some embodiments, R is C₁₋₄ aliphatic. In some embodiments, R is C₁₋₄ alkyl. In some embodiments, R is C₁₋₃ aliphatic. In some embodiments, R is C₁₋₃ alkyl. In some embodiments, R is C₂ aliphatic. In some embodiments, R is methyl. In some embodiments, R is C₁₋₆ haloaliphatic. In some embodiments, R is C₁₋₆ haloalkyl. In some embodiments, R is C₁₋₅ haloaliphatic. In some embodiments, R is C₁₋₅ haloalkyl. In some embodiments, R is C₁₋₄ haloaliphatic. In some embodiments, R is C₁₋₄ haloalkyl. In some embodiments, R is C₁₋₃ haloaliphatic. In some embodiments, R is C₁₋₃ haloalkyl. In some embodiments, R is C₂ haloaliphatic. In some embodiments, R is methyl substituted with one or more halogen. In some embodiments, R is —CF₃. In some embodiments, L^{s} is optionally substituted —CH═CH—. In some embodiments, L^{s} is optionally substituted (E)-CH═CH—. In some embodiments, L^{s} is optionally substituted (Z)—CH═CH—. In some embodiments, L^{s} is —C≡C—.

In some embodiments, LS comprises at least one phosphorus atom. In some embodiments, at least one methylene unit of L^{s} is replaced with —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—.

In some embodiments, L^{s} is -Cy-. In some embodiments, -Cy- is optionally substituted monocyclic or bicyclic 3-20 membered heterocyclyl ring having 1-5 heteroatoms. In some embodiments, -Cy- is optionally substituted monocyclic or bicyclic 5-20 membered heterocyclyl ring having 1-5 heteroatoms, wherein at least one heteroatom is oxygen. In some embodiments, -Cy- is optionally substituted bivalent tetrahydrofuran ring. In some embodiments, -Cy- is an optionally substituted furanose moiety.

As described herein, each L is independently a covalent bond, or a bivalent, optionally substituted, linear or branched group selected from a C₁₋₃₀ aliphatic group and a C₁₋₃₀ heteroaliphatic group having 1-10 heteroatoms independently selected from oxygen, nitrogen, sulfur, phosphorus, boron and silicon, wherein one or more methylene units are optionally and independently replaced with C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—; and one or more carbon atoms are optionally and independently replaced with Cy^{L}.

In some embodiments, L is a covalent bond, or a bivalent, optionally substituted, linear or branched group selected from a C₁₋₃₀ aliphatic group and a C₁₋₃₀ heteroaliphatic group having 1-10 heteroatoms independently selected from oxygen, nitrogen, sulfur, phosphorus and silicon, wherein one or more methylene units are optionally and independently replaced by an optionally substituted group selected from C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more carbon atoms are optionally and independently replaced with Cy^{L}. In some embodiments, L is a covalent bond, or a bivalent, optionally substituted, linear or branched C₁₋₃₀ aliphatic group, wherein one or more methylene units are optionally and independently replaced by an optionally substituted group selected from C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more carbon atoms are optionally and independently replaced with Cy^{L}. In some embodiments, L is a covalent bond, or a bivalent, optionally substituted, linear or branched C₁₋₃₀ heteroaliphatic group having 1-10 heteroatoms independently selected from oxygen, nitrogen, sulfur, phosphorus and silicon, wherein one or more methylene units are optionally and independently replaced by an optionally substituted group selected from C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more carbon atoms are optionally and independently replaced with Cy^{L}. In some embodiments, L is a covalent bond, or a bivalent, optionally substituted, linear or branched group selected from a C₁₋₃₀ aliphatic group and a C₁₋₃₀ heteroaliphatic group having 1-10 heteroatoms independently selected from oxygen, nitrogen, sulfur, phosphorus and silicon, wherein one or more methylene units are optionally and independently replaced by an optionally substituted group selected from C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—, and one or more carbon atoms are optionally and independently replaced with Cy^{L}. In some embodiments, L is a covalent bond, or a bivalent, optionally substituted, linear or branched group selected from a C₁₋₁₀ aliphatic group and a C₁₋₁₀ heteroaliphatic group having 1-5 heteroatoms independently selected from oxygen, nitrogen, sulfur, phosphorus and silicon, wherein one or more methylene units are optionally and independently replaced by an optionally substituted group selected from C₁₋₆ alkylene, C₁₋₆ alkenylene, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, and —C(O)O—, and one or more carbon atoms are optionally and independently replaced with Cy^{L}. In some embodiments, L is a covalent bond, or a bivalent, optionally substituted, linear or branched group selected from a C₁₋₁₀ aliphatic group and a C₁₋₁₀ heteroaliphatic group having 1-5 heteroatoms independently selected from oxygen, nitrogen, sulfur, phosphorus and silicon, wherein one or more methylene units are optionally and independently replaced by an optionally substituted group selected from —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, and —C(O)O—.

In some embodiments, L is a covalent bond. In some embodiments, L is optionally substituted bivalent C₁₋₃₀ aliphatic. In some embodiments, L is optionally substituted bivalent C₁₋₃₀ heteroaliphatic having 1-10 heteroatoms independently selected from boron, oxygen, nitrogen, sulfur, phosphorus and silicon.

In some embodiments, aliphatic moieties, e.g. those of L, R, etc., either monovalent or bivalent or multivalent, and can contain any number of carbon atoms (before any optional substitution) within its range, e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, etc. In some embodiments, heteroaliphatic moieties, e.g. those of L, R, etc., either monovalent or bivalent or multivalent, and can contain any number of carbon atoms (before any optional substitution) within its range, e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, etc.

In some embodiments, one or more methylene unit is optionally and independently substituted with —O—, —S—, —N(R′)—, —C(O)—, —S(O)—, —S(O)₂—, —P(O)(OR′)—, —P(O)(SR′)—, —P(S)(OR′)—, or —P(S)(OR′)—. In some embodiments, a methylene unit is replaced with —O—. In some embodiments, a methylene unit is replaced with —S—. In some embodiments, a methylene unit is replaced with —N(R′)—. In some embodiments, a methylene unit is replaced with —C(O)—. In some embodiments, a methylene unit is replaced with —S(O)—. In some embodiments, a methylene unit is replaced with —S(O)₂—. In some embodiments, a methylene unit is replaced with —P(O)(OR′)—. In some embodiments, a methylene unit is replaced with —P(O)(SR′)—. In some embodiments, a methylene unit is replaced with —P(O)(R′)—. In some embodiments, a methylene unit is replaced with —P(O)(NR′)—. In some embodiments, a methylene unit is replaced with —P(S)(OR′)—. In some embodiments, a methylene unit is replaced with —P(S)(SR′)—. In some embodiments, a methylene unit is replaced with —P(S)(R′)—. In some embodiments, a methylene unit is replaced with —P(S)(NR′)—. In some embodiments, a methylene unit is replaced with —P(R′)—. In some embodiments, a methylene unit is replaced with —P(OR′)—. In some embodiments, a methylene unit is replaced with —P(SR′)—. In some embodiments, a methylene unit is replaced with —P(NR′)—. In some embodiments, a methylene unit is replaced with —P(OR′)[B(R′)₃]—. In some embodiments, one or more methylene unit is optionally and independently substituted with —O—, —S—, —N(R′)—, —C(O)—, —S(O)—, —S(O)₂—, —P(O)(OR′)—, —P(O)(SR′)—, —P(S)(OR′)—, or —P(S)(OR′)—. In some embodiments, a methylene unit is replaced with —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, each of which may independently be an internucleotidic linkage.

In some embodiments, L, e.g., when connected to R, is —CH₂—. In some embodiments, L is —C(R)₂—, wherein at least one R is not hydrogen. In some embodiments, L is —CHR—. In some embodiments, R is hydrogen. In some embodiments, L is —CHR—, wherein R is not hydrogen. In some embodiments, C of —CHR— is chiral. In some embodiments, L is —(R)—CHR—, wherein C of —CHR— is chiral. In some embodiments, L is —(S)—CHR—, wherein C of —CHR— is chiral. In some embodiments, R is optionally substituted C₁₋₆ aliphatic. In some embodiments, R is optionally substituted C₁₋₆ alkyl. In some embodiments, R is optionally substituted C₁₋₅ aliphatic. In some embodiments, R is optionally substituted C₁₋₅ alkyl. In some embodiments, R is optionally substituted C₁₋₄ aliphatic. In some embodiments, R is optionally substituted C₁₋₄ alkyl. In some embodiments, R is optionally substituted C₁₋₃ aliphatic. In some embodiments, R is optionally substituted C₁₋₃ alkyl. In some embodiments, R is optionally substituted C₂ aliphatic. In some embodiments, R is optionally substituted methyl. In some embodiments, R is C₁₋₆ aliphatic. In some embodiments, R is C₁₋₆ alkyl. In some embodiments, R is C₁₋₅ aliphatic. In some embodiments, R is C₁₋₅ alkyl. In some embodiments, R is C₁₋₄ aliphatic. In some embodiments, R is C₁₋₄ alkyl. In some embodiments, R is C₁₋₃ aliphatic. In some embodiments, R is C₁₋₃ alkyl. In some embodiments, R is C₂ aliphatic. In some embodiments, R is methyl. In some embodiments, R is C₁₋₆ haloaliphatic. In some embodiments, R is C₁₋₆ haloalkyl. In some embodiments, R is C₁₋₅ haloaliphatic. In some embodiments, R is C₁₋₅ haloalkyl. In some embodiments, R is C₁₋₄ haloaliphatic. In some embodiments, R is C₁₋₄ haloalkyl. In some embodiments, R is C₁₋₃ haloaliphatic. In some embodiments, R is C₁₋₃ haloalkyl. In some embodiments, R is C₂ haloaliphatic. In some embodiments, R is methyl substituted with one or more halogen. In some embodiments, R is —CF₃. In some embodiments, L is optionally substituted —CH═CH—. In some embodiments, L is optionally substituted (E)-CH═CH—. In some embodiments, L is optionally substituted (Z)—CH═CH—. In some embodiments, L is —C≡C—.

In some embodiments, L comprises at least one phosphorus atom. In some embodiments, at least one methylene unit of L is replaced with —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—.

In some embodiments, Cy^{L} is an optionally substituted tetravalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus, boron and silicon.

In some embodiments, Cy^{L} is monocyclic. In some embodiments, Cy^{L} is bicyclic. In some embodiments, Cy^{L} is polycyclic.

In some embodiments, Cy^{L} is saturated. In some embodiments, Cy^{L} is partially unsaturated. In some embodiments, Cy^{L} is aromatic. In some embodiments, Cy^{L} is or comprises a saturated ring moiety. In some embodiments, Cy^{L} is or comprises a partially unsaturated ring moiety. In some embodiments, Cy^{L} is or comprises an aromatic ring moiety.

In some embodiments, Cy^{L} is an optionally substituted C₃₋₂₀ cycloaliphatic ring as described in the present disclosure (for example, those described for R but tetravalent). In some embodiments, a ring is an optionally substituted saturated C₃₋₂₀ cycloaliphatic ring. In some embodiments, a ring is an optionally substituted partially unsaturated C₃₋₂₀ cycloaliphatic ring. A cycloaliphatic ring can be of various sizes as described in the present disclosure. In some embodiments, a ring is 3, 4, 5, 6, 7, 8, 9, or 10-membered. In some embodiments, a ring is 3-membered. In some embodiments, a ring is 4-membered. In some embodiments, a ring is 5-membered. In some embodiments, a ring is 6-membered. In some embodiments, a ring is 7-membered. In some embodiments, a ring is 8-membered. In some embodiments, a ring is 9-membered. In some embodiments, a ring is 10-membered. In some embodiments, a ring is an optionally substituted cyclopropyl moiety. In some embodiments, a ring is an optionally substituted cyclobutyl moiety. In some embodiments, a ring is an optionally substituted cyclopentyl moiety. In some embodiments, a ring is an optionally substituted cyclohexyl moiety. In some embodiments, a ring is an optionally substituted cycloheptyl moiety. In some embodiments, a ring is an optionally substituted cyclooctanyl moiety. In some embodiments, a cycloaliphatic ring is a cycloalkyl ring. In some embodiments, a cycloaliphatic ring is monocyclic. In some embodiments, a cycloaliphatic ring is bicyclic. In some embodiments, a cycloaliphatic ring is polycyclic. In some embodiments, a ring is a cycloaliphatic moiety as described in the present disclosure for R with more valences.

In some embodiments, Cy^{L} is an optionally substituted 6-20 membered aryl ring. In some embodiments, a ring is an optionally substituted tetravalent phenyl moiety. In some embodiments, a ring is a tetravalent phenyl moiety. In some embodiments, a ring is an optionally substituted naphthalene moiety. A ring can be of different size as described in the present disclosure. In some embodiments, an aryl ring is 6-membered. In some embodiments, an aryl ring is 10-membered. In some embodiments, an aryl ring is 14-membered. In some embodiments, an aryl ring is monocyclic. In some embodiments, an aryl ring is bicyclic. In some embodiments, an aryl ring is polycyclic. In some embodiments, a ring is an aryl moiety as described in the present disclosure for R with more valences.

In some embodiments, Cy^{L} is an optionally substituted 5-20 membered heteroaryl ring having 1-10 heteroatoms independently selected from oxygen, nitrogen, sulfur, phosphorus and silicon. In some embodiments, Cy^{L} is an optionally substituted 5-20 membered heteroaryl ring having 1-10 heteroatoms independently selected from oxygen, nitrogen, and sulfur. In some embodiments, as described in the present disclosure, heteroaryl rings can be of various sizes and contain various numbers and/or types of heteroatoms. In some embodiments, a heteroaryl ring contains no more than one heteroatom. In some embodiments, a heteroaryl ring contains more than one heteroatom. In some embodiments, a heteroaryl ring contains no more than one type of heteroatom. In some embodiments, a heteroaryl ring contains more than one type of heteroatoms. In some embodiments, a heteroaryl ring is 5-membered. In some embodiments, a heteroaryl ring is 6-membered. In some embodiments, a heteroaryl ring is 8-membered. In some embodiments, a heteroaryl ring is 9-membered. In some embodiments, a heteroaryl ring is 10-membered. In some embodiments, a heteroaryl ring is monocyclic. In some embodiments, a heteroaryl ring is bicyclic. In some embodiments, a heteroaryl ring is polycyclic. In some embodiments, a heteroaryl ring is a nucleobase moiety, e.g., A, T, C, G, U, etc. In some embodiments, a ring is a heteroaryl moiety as described in the present disclosure for R with more valences.

In some embodiments, Cy^{L} is a 3-20 membered heterocyclyl ring having 1-10 heteroatoms independently selected from oxygen, nitrogen, sulfur, phosphorus and silicon. In some embodiments, Cy^{L} is a 3-20 membered heterocyclyl ring having 1-10 heteroatoms independently selected from oxygen, nitrogen, and sulfur. In some embodiments, a heterocyclyl ring is saturated. In some embodiments, a heterocyclyl ring is partially unsaturated. A heterocyclyl ring can be of various sizes as described in the present disclosure. In some embodiments, a ring is 3, 4, 5, 6, 7, 8, 9, or 10-membered. In some embodiments, a ring is 3-membered. In some embodiments, a ring is 4-membered. In some embodiments, a ring is 5-membered. In some embodiments, a ring is 6-membered. In some embodiments, a ring is 7-membered. In some embodiments, a ring is 8-membered. In some embodiments, a ring is 9-membered. In some embodiments, a ring is 10-membered. Heterocyclyl rings can contain various numbers and/or types of heteroatoms. In some embodiments, a heterocyclyl ring contains no more than one heteroatom. In some embodiments, a heterocyclyl ring contains more than one heteroatom. In some embodiments, a heterocyclyl ring contains no more than one type of heteroatom. In some embodiments, a heterocyclyl ring contains more than one type of heteroatoms. In some embodiments, a heterocyclyl ring is monocyclic. In some embodiments, a heterocyclyl ring is bicyclic. In some embodiments, a heterocyclyl ring is polycyclic. In some embodiments, a ring is a heterocyclyl moiety as described in the present disclosure for R with more valences.

As readily appreciated by a person having ordinary skill in the art, many suitable ring moieties are extensively described in and can be used in accordance with the present disclosure, for example, those described for R (which may have more valences for Cy^{L}).

In some embodiments, Cy^{L} is a sugar moiety in a nucleic acid. In some embodiments, Cy^{L} is an optionally substituted furanose moiety. In some embodiments, Cy^{L} is a pyranose moiety. In some embodiments, Cy^{L} is an optionally substituted furanose moiety found in DNA. In some embodiments, Cy^{L} is an optionally substituted furanose moiety found in RNA. In some embodiments, Cy^{L} is an optionally substituted 2′-deoxyribofuranose moiety. In some embodiments, Cy^{L} is an optionally substituted ribofuranose moiety. In some embodiments, substitutions provide sugar modifications as described in the present disclosure. In some embodiments, an optionally substituted 2′-deoxyribofuranose moiety and/or an optionally substituted ribofuranose moiety comprise substitution at a 2′-position. In some embodiments, a 2′-position is a 2′-modification as described in the present disclosure. In some embodiments, a 2′-modification is —F. In some embodiments, a 2′-modification is —OR, wherein R is as described in the present disclosure. In some embodiments, R is not hydrogen. In some embodiments, Cy^{L} is a modified sugar moiety, such as a sugar moiety in LNA, alpha-L-LNA or GNA. In some embodiments, Cy^{L} is a modified sugar moiety, such as a sugar moiety in ENA. In some embodiments, Cy^{L} is a terminal sugar moiety of an oligonucleotide, connecting an internucleotidic linkage and a nucleobase. In some embodiments, Cy^{L} is a terminal sugar moiety of an oligonucleotide, for example, when that terminus is connected to a solid support optionally through a linker. In some embodiments, Cy^{L} is a sugar moiety connecting two internucleotidic linkages and a nucleobase. Example sugars and sugar moieties are extensively described in the present disclosure.

In some embodiments, Cy^{L} is a nucleobase moiety. In some embodiments, a nucleobase is a natural nucleobase, such as A, T, C, G, U, etc. In some embodiments, a nucleobase is a modified nucleobase. In some embodiments, Cy^{L} is optionally substituted nucleobase moiety selected from A, T, C, G, U, and 5mC. Example nucleobases and nucleobase moieties are extensively described in the present disclosure.

In some embodiments, two Cy^{L} moieties are bonded to each other, wherein one Cy^{L} is a sugar moiety and the other is a nucleobase moiety. In some embodiments, such a sugar moiety and nucleobase moiety forms a nucleoside moiety. In some embodiments, a nucleoside moiety is natural. In some embodiments, a nucleoside moiety is modified. In some embodiments, Cy^{L} is an optionally substituted natural nucleoside moiety selected from adenosine, 5-methyluridine, cytidine, guanosine, uridine, 5-methylcytidine, 2′-deoxyadenosine, thymidine, 2′-deoxycytidine, 2′-deoxyguanosine, 2′-deoxyuridine, and 5-methyl-2′-deoxycytidine. Example nucleosides and nucleosides moieties are extensive described in the present disclosure.

In some embodiments, for example in LS, Cy^{L} is an optionally substituted nucleoside moiety bonded to an internucleotidic linkage, for example, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, —OP(OR′)[B(R′)₃]O—, etc., which may form an optionally substituted nucleotidic unit. Example nucleotides and nucleosides moieties are extensive described in the present disclosure. In some embodiments, -Cy- is an optionally substituted bivalent 3-30 membered carbocyclylene. In some embodiments, -Cy- is an optionally substituted bivalent 6-30 membered arylene. In some embodiments, -Cy- is an optionally substituted bivalent 5-30 membered heteroarylene having 1-10 heteroatoms independently selected from oxygen, nitrogen and sulfur. In some embodiments, -Cy- is an optionally substituted bivalent 3-30 membered heterocyclylene having 1-10 heteroatoms independently selected from oxygen, nitrogen, sulfur, phosphorus and silicon. In some embodiments, -Cy- is an optionally substituted bivalent 5-30 membered heteroarylene having 1-5 heteroatoms independently selected from oxygen, nitrogen and sulfur. In some embodiments, -Cy- is an optionally substituted bivalent 3-30 membered heterocyclylene having 1-5 heteroatoms independently selected from oxygen, nitrogen, sulfur, phosphorus and silicon.

In some embodiments, each Ring A^{s} is independently an optionally substituted 3-20 membered monocyclic, bicyclic or polycyclic ring having 0-10 heteroatoms independently selected from oxygen, nitrogen, sulfur, phosphorus and silicon. In some embodiments, Ring A^{s} is an optionally substituted ring, which ring is as described in the present disclosure. In some embodiments, Ring A^{s} is optionally substituted

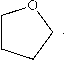

In some embodiments, Ring A^{s} is

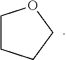

In some embodiments, Ring A^{s} is optionally substituted

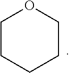

In some embodiments, Ring A^{s} is

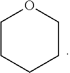

In some embodiments, Ring A^{s} is a bicyclic ring, e.g., a bicyclic ring in bicyclic sugars. In some embodiments, Ring A^{s} is a polycyclic ring.

In some embodiments,

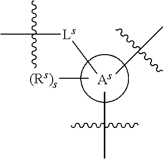

has the structure of

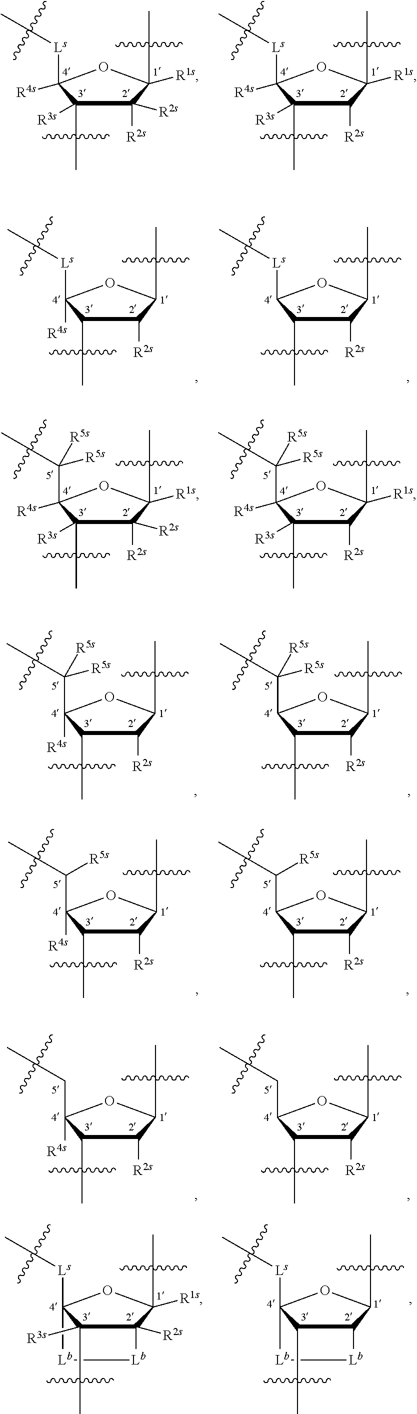

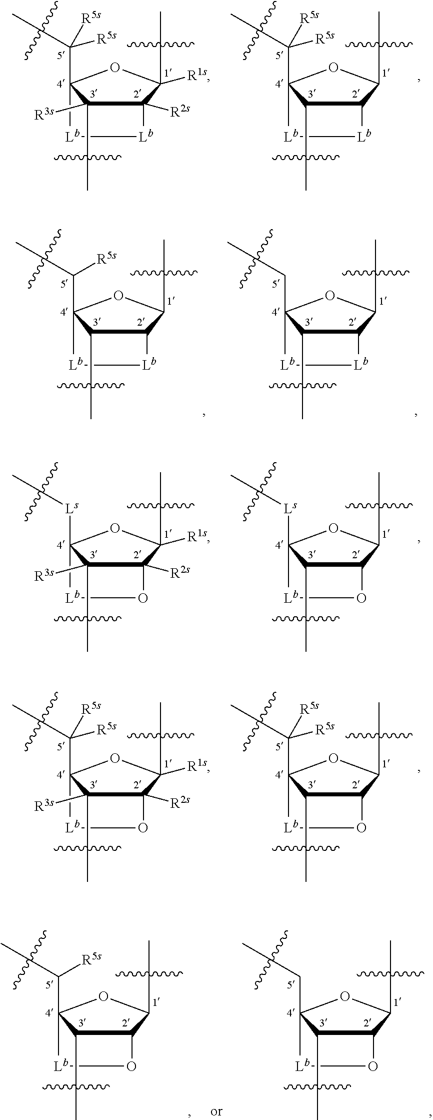

wherein each L^{b} is independently L, and each other variable is independently as described in the present disclosure. Example embodiments include those described for Sugars. In some embodiments, one L^{b} is —O—, —S— or —N(R′)—. In some embodiments, the L^{b} connect to the 2′ carbon is —O—, —S— or —N(R′)—. In some embodiments, L^{b} is —C(R)₂—. In some embodiments, the L^{b} connect to the 4′ carbon is —C(R)₂—. In some embodiments, —C(R)₂— is —CHR—. In some embodiments, both L^{b} are independently —C(R)₂—.

In some embodiments, each of R^{s}, R^{2s}, R^{3s}, R^{4s}, and R^{5s} is independently R^{s}, wherein R^{s} is as described in the present disclosure.

In some embodiments, R^{1s} is R^{s} wherein R^{s} is as described in the present disclosure. In some embodiments, R^{1s} is at 1′-position (BA is at 1′-position). In some embodiments, R^{1s} is —H. In some embodiments, R^{1s} is —F. In some embodiments, R^{1s} is —Cl. In some embodiments, R^{1s} is —Br. In some embodiments, R^{1s} is —I. In some embodiments, R^{1s} is —CN. In some embodiments, R^{1s} is —N₃. In some embodiments, R^{1s} is —NO. In some embodiments, R^{1s} is —NO₂. In some embodiments, R¹⁵ is -L-R′. In some embodiments, R^{1s} is —R′. In some embodiments, R^{1s} is -L-OR′. In some embodiments, R¹⁵ is —OR′. In some embodiments, R^{1s} is -L-SR′. In some embodiments, R^{1s} is —SR′. In some embodiments, R^{1s} is L-L-N(R′)₂. In some embodiments, R^{1s} is —N(R′)₂. In some embodiments, R^{1s} is —OR^{•}, wherein R′ is optionally substituted C₁₋₆ aliphatic. In some embodiments, R^{1s} is —OR^{•}, wherein R′ is optionally substituted C₁₋₆ alkyl. In some embodiments, R^{1s} is —OMe. In some embodiments, R^{1s} is -MOE. In some embodiments, R^{1s} is hydrogen. In some embodiments, R^{s} at one 1′-position is hydrogen, and R^{s} at the other 1′-position is not hydrogen as described herein. In some embodiments, R^{s} at both 1′-positions are hydrogen. In some embodiments, R^{s} at one 1′-position is hydrogen, and the other 1′-position is connected to an internucleotidic linkage. In some embodiments, R^{1s} is —F. In some embodiments, R^{1s} is —Cl. In some embodiments, R^{1s} is —Br. In some embodiments, R^{1s} is —I. In some embodiments, R^{1s} is —CN. In some embodiments, R^{1s} is —N₃. In some embodiments, R^{1s} is —NO. In some embodiments, R^{1s} is —NO₂. In some embodiments, R^{1s} is -L-R′. In some embodiments, R^{1s} is —R′. In some embodiments, R^{1s} is -L-OR′. In some embodiments, R^{1s} is —OR′. In some embodiments, R^{1s} is -L-SR′. In some embodiments, R^{1s} is —SR′. In some embodiments, R^{1s} is -L-N(R′)₂. In some embodiments, R^{1s} is —N(R′)₂. In some embodiments, R^{1s} is —OR′, wherein R′ is optionally substituted C₁₋₆ aliphatic. In some embodiments, R^{1s} is —OR′, wherein R′ is optionally substituted C₁₋₆ alkyl. In some embodiments, R^{1s} is —OH. In some embodiments, R^{1s} is —OMe. In some embodiments, R^{1s} is -MOE. In some embodiments, R^{1s} is hydrogen. In some embodiments, one R^{1s} at a 1′-position is hydrogen, and the other R^{1s} at the other 1′-position is not hydrogen as described herein. In some embodiments, R^{1s} at both 1′-positions are hydrogen. In some embodiments, R^{1s} is —O-L^{s}-OR′. In some embodiments, R^{1s} is —O-L^{s}-OR′, wherein L^{s} is optionally substituted C₁₋₆ alkylene, and R′ is optionally substituted C₁₋₆ aliphatic. In some embodiments, R^{1s} is —O-(optionally substituted C₁₋₆ alkylene)-OR′. In some embodiments, R^{1s} is —O-(optionally substituted C₁₋₆ alkylene)-OR′, wherein R′ is optionally substituted C₁₋₆ alkyl. In some embodiments, R^{1s} is —OCH₂CH₂OMe.

In some embodiments, R^{2s} is R^{s} wherein R^{s} is as described in the present disclosure. In some embodiments, if there are two R^{2s} at the 2′-position, one R^{2s} is —H and the other is not. In some embodiments, R^{2s} is at 2′-position (BA is at 1′-position). In some embodiments, R^{2s} is —H. In some embodiments, R^{2s} is —F. In some embodiments, R^{2s} is —Cl. In some embodiments, R^{2s} is —Br. In some embodiments, R^{2s} is —I. In some embodiments, R^{2s} is —CN. In some embodiments, R^{2s} is —N₃. In some embodiments, R^{2s} is —NO. In some embodiments, R^{2s} is —NO₂. In some embodiments, R^{2s} is -L-R′. In some embodiments, R^{2s} is —R′. In some embodiments, R^{2s} is -L-OR′. In some embodiments, R^{2s} is —OR′. In some embodiments, R^{2s} is -L-SR′. In some embodiments, R^{2s} is —SR′. In some embodiments, R^{2s} is L-L-N(R′)₂. In some embodiments, R^{2s} is —N(R′)₂. In some embodiments, R^{2s} is —OR′, wherein R′ is optionally substituted C₁₋₆ aliphatic. In some embodiments, R^{2s} is —OR′, wherein R′ is optionally substituted C₁₋₆ alkyl. In some embodiments, R^{2s} is —OMe. In some embodiments, R^{2s} is -MOE. In some embodiments, R^{2s} is hydrogen. In some embodiments, R^{s} at one 2′-position is hydrogen, and R^{s} at the other 2′-position is not hydrogen as described herein. In some embodiments, R^{s} at both 2′-positions are hydrogen. In some embodiments, R^{s} at one 2′-position is hydrogen, and the other 2′-position is connected to an internucleotidic linkage. In some embodiments, R^{2s} is —F. In some embodiments, R^{2s} is —Cl. In some embodiments, R^{2s} is —Br. In some embodiments, R^{2s} is —I. In some embodiments, R^{2s} is —CN. In some embodiments, R^{2s} is —N₃. In some embodiments, R^{2s} is —NO. In some embodiments, R^{2s} is —NO₂. In some embodiments, R^{2s} is -L-R′. In some embodiments, R^{2s} is —R′. In some embodiments, R^{2s} is -L-OR′. In some embodiments, R^{2s} is —OR′. In some embodiments, R^{2s} is -L-SR′. In some embodiments, R^{2s} is —SR′. In some embodiments, R^{2s} is -L-N(R′)₂. In some embodiments, R^{2s} is —N(R′)₂. In some embodiments, R^{2s} is —OR^{•}, wherein R′ is optionally substituted C₁₋₆ aliphatic. In some embodiments, R^{2s} is —OR′, wherein R′ is optionally substituted C₁₋₆ alkyl. In some embodiments, R^{2s} is —OH. In some embodiments, R^{2s} is —OMe. In some embodiments, R^{2s} is -MOE. In some embodiments, R^{2s} is hydrogen. In some embodiments, one R^{2s} at a 2′-position is hydrogen, and the other R^{2s} at the other 2′-position is not hydrogen as described herein. In some embodiments, R^{2s} at both 2′-positions are hydrogen. In some embodiments, R^{2s} is —O-L^{s}-OR′. In some embodiments, R^{2s} is —O-L^{s}-OR′, wherein L^{s} is optionally substituted C₁₋₆ alkylene, and R′ is optionally substituted C₁₋₆ aliphatic. In some embodiments, R^{2s} is —O-(optionally substituted C₁₋₆ alkylene)-OR′. In some embodiments, R^{2s} is —O-(optionally substituted C₁₋₆ alkylene)-OR′, wherein R′ is optionally substituted C₁₋₆ alkyl. In some embodiments, R^{2s} is —OCH₂CH₂OMe.

In some embodiments, R^{3s} is R^{s} wherein R^{s} is as described in the present disclosure. In some embodiments, R^{3s} is at 3′-position (BA is at 1′-position). In some embodiments, R^{3s} is —H. In some embodiments, R^{3s} is —F. In some embodiments, R^{3s} is —Cl. In some embodiments, R^{3s} is —Br. In some embodiments, R^{3s} is —I. In some embodiments, R^{3s} is —CN. In some embodiments, R^{3s} is —N₃. In some embodiments, R^{3s} is —NO. In some embodiments, R^{3s} is —NO₂. In some embodiments, R^{3s} is -L-R′. In some embodiments, R^{3s} is —R′. In some embodiments, R^{3s} is -L-OR′. In some embodiments, R^{3s} is —OR′. In some embodiments, R^{3s} is -L-SR′. In some embodiments, R^{3s} is —SR′. In some embodiments, R^{3s} is -L-N(R′)₂. In some embodiments, R^{3s} is —N(R′)₂. In some embodiments, R^{3s} is —OR′, wherein R′ is optionally substituted C₁₋₆ aliphatic. In some embodiments, R^{3s} is —OR′, wherein R′ is optionally substituted C₁₋₆ alkyl. In some embodiments, R^{3s} is —OMe. In some embodiments, R^{3s} is -MOE. In some embodiments, R^{3s} is hydrogen. In some embodiments, R^{s} at one 3′-position is hydrogen, and R^{s} at the other 3′-position is not hydrogen as described herein. In some embodiments, R^{s} at both 3′-positions are hydrogen. In some embodiments, R^{s} at one 3′-position is hydrogen, and the other 3′-position is connected to an internucleotidic linkage. In some embodiments, R^{3s} is —F. In some embodiments, R^{3s} is —Cl. In some embodiments, R^{3s} is —Br. In some embodiments, R^{3s} is —I. In some embodiments, R^{3s} is —CN. In some embodiments, R^{3s} is —N₃. In some embodiments, R^{3s} is —NO. In some embodiments, R^{3s} is —NO₂. In some embodiments, R^{3s} is -L-R′. In some embodiments, R^{3s} is —R′. In some embodiments, R^{3s} is -L-OR′. In some embodiments, R^{3s} is —OR′. In some embodiments, R^{3s} is -L-SR′. In some embodiments, R^{3s} is —SR′. In some embodiments, R^{3s} is L-L-N(R′)₂. In some embodiments, R^{3s} is —N(R′)₂. In some embodiments, R^{3s} is —OR^{•}, wherein R′ is optionally substituted C₁₋₆ aliphatic. In some embodiments, R^{3s} is —OR^{•}, wherein R′ is optionally substituted C₁₋₆ alkyl. In some embodiments, R^{3s} is —OH. In some embodiments, R^{3s} is —OMe. In some embodiments, R^{3s} is -MOE. In some embodiments, R^{3s} is hydrogen.

In some embodiments, R^{4s} is R^{s} wherein R^{s} is as described in the present disclosure. In some embodiments, R^{4s} is at 4′-position (BA is at 1′-position). In some embodiments, R^{4s} is —H. In some embodiments, R^{4s} is —F. In some embodiments, R^{4s} is —Cl. In some embodiments, R^{4s} is —Br. In some embodiments, R^{4s} is —I. In some embodiments, R^{4s} is —CN. In some embodiments, R^{4s} is —N₃. In some embodiments, R^{4s} is —NO. In some embodiments, R^{4s} is —NO₂. In some embodiments, R^{4s} is -L-R′. In some embodiments, R^{4s} is —R′. In some embodiments, R^{4s} is -L-OR′. In some embodiments, R^{4s} is —OR′. In some embodiments, R^{4s} is -L-SR′. In some embodiments, R^{4s} is —SR′. In some embodiments, R^{4s} is -L-N(R′)₂. In some embodiments, R^{4s} is —N(R′)₂. In some embodiments, R^{4s} is —OR′, wherein R′ is optionally substituted C₁₋₆ aliphatic. In some embodiments, R^{4s} is —OR′, wherein R′ is optionally substituted C₁₋₆ alkyl. In some embodiments, R^{4s} is —OMe. In some embodiments, R^{4s} is -MOE. In some embodiments, R^{4s} is hydrogen. In some embodiments, R^{s} at one 4′-position is hydrogen, and R^{s} at the other 4′-position is not hydrogen as described herein. In some embodiments, R^{s} at both 4′-positions are hydrogen. In some embodiments, R^{s} at one 4′-position is hydrogen, and the other 4′-position is connected to an internucleotidic linkage. In some embodiments, R^{4s} is —F. In some embodiments, R^{4s} is —Cl. In some embodiments, R^{4s} is —Br. In some embodiments, R^{4s} is —I. In some embodiments, R^{4s} is —CN. In some embodiments, R^{4s} is —N₃. In some embodiments, R^{4s} is —NO. In some embodiments, R^{4s} is —NO₂. In some embodiments, R^{4s} is -L-R′. In some embodiments, R^{4s} is —R′. In some embodiments, R^{4s} is -L-OR′. In some embodiments, R^{4s} is —OR′. In some embodiments, R^{4s} is -L-SR′. In some embodiments, R^{4s} is —SR′. In some embodiments, R^{4s} is L-L-N(R′)₂. In some embodiments, R^{4s} is —N(R′)₂. In some embodiments, R^{4s} is —OR′, wherein R′ is optionally substituted C₁₋₆ aliphatic. In some embodiments, R^{4s} is —OR′, wherein R′ is optionally substituted C₁₋₆ alkyl. In some embodiments, R^{4s} is —OH. In some embodiments, R^{4s} is —OMe. In some embodiments, R^{4s} is -MOE. In some embodiments, R^{4s} is hydrogen.

In some embodiments, R^{5s} is R^{s} wherein R^{s} is as described in the present disclosure. In some embodiments, R^{5s} is R′ wherein R′ is as described in the present disclosure. In some embodiments, R^{5s} is —H. In some embodiments, two or more R^{5s} are connected to the same carbon atom, and at least one is not —H. In some embodiments, R^{5s} is not —H. In some embodiments, R^{5s} is —F. In some embodiments, R^{5s} is —Cl. In some embodiments, R^{s} is —Br. In some embodiments, R^{s} is —I. In some embodiments, R^{5s} is —CN. In some embodiments, R^{5s} is —N₃. In some embodiments, R^{s} is —NO. In some embodiments, R^{5s} is —NO₂. In some embodiments, R^{5s} is -L-R′. In some embodiments, R^{5s} is —R′. In some embodiments, R^{5s} is -L-OR′. In some embodiments, R^{5s} is —OR′. In some embodiments, R^{5s} is -L-SR′. In some embodiments, R^{5s} is —SR′. In some embodiments, R^{5s} is L-L-N(R′)₂. In some embodiments, R^{5s} is —N(R′)₂. In some embodiments, R^{5s} is —OR′, wherein R′ is optionally substituted C₁₋₆ aliphatic. In some embodiments, R^{5s} is —OR′, wherein R′ is optionally substituted C₁₋₆ alkyl. In some embodiments, R^{5s} is —OH. In some embodiments, R^{5s} is —OMe. In some embodiments, R^{5s} is -MOE. In some embodiments, R^{5s} is hydrogen.

In some embodiments, R^{5s} is optionally substituted C₁₋₆ aliphatic as described in the present disclosure, e.g., C₁₋₆ aliphatic embodiments described for R or other variables. In some embodiments, R^{5s} is optionally substituted C₁₋₆ alkyl. In some embodiments, R^{5s} is methyl. In some embodiments, R^{5s} is ethyl.

In some embodiments, R^{5s} is a protected hydroxyl group suitable for oligonucleotide synthesis. In some embodiments, R^{5s} is —OR^{•}, wherein R′ is optionally substituted C₁₋₆ aliphatic. In some embodiments, R^{5s} is DMTrO—. Example protecting groups are widely known for use in accordance with the present disclosure. For additional examples, see Greene, T. W.; Wuts, P. G. M. Protective Groups in Organic Synthesis, 2nd ed.; Wiley: New York, 1991, and WO 2011/005761, WO 2013/012758, WO 2014/012081, WO 2015/107425, WO 2010/064146, WO 2014/010250, WO 2011/108682, WO 2012/039448, and WO 2012/073857.

In some embodiments, two or more of R^{1s}, R^{2s}, R^{3s}, R^{4s}, and R^{5s} are R and can be taken together with intervening atom(s) to form a ring as described in the present disclosure. In some embodiments, R^{2s} and R^{4s} are R taken together to form a ring, and a sugar moiety can be a bicyclic sugar moiety, e.g., a LNA sugar moiety.

In some embodiments, L^{s} is —C(R^{5a})₂—, wherein each R^{5s} is independently as described in the present disclosure. In some embodiments, one of R^{5s} is H and the other is not H. In some embodiments, none of R^{5s} is H. In some embodiments, L^{s} is —CHR^{5s}—, wherein each R^{5s} is independently as described in the present disclosure. In some embodiments, —C(R^{5s})₂— is 5′-C, optionally substituted, of a sugar moiety. In some embodiments, the C of —C(R^{5s})₂— is of R configuration. In some embodiments, the C of —C(R⁵)₂— is of S configuration. As described in the present disclosure, in some embodiments, R^{5s} is optionally substituted C₁₋₆ aliphatic; in some embodiments, R^{5s} is methyl.

In some embodiments, provided compounds comprise one or more bivalent or multivalent optionally substituted rings, e.g., Ring A^{s}, Ring A^{L}, Cy^{L}, -Cy-, those formed by two or more R groups (R and (combinations of) variables that can be R) taken together, etc. In some embodiments, a ring is a cycloaliphatic, aryl, heteroaryl, or heterocyclyl group as described for R but bivalent or multivalent. As appreciated by those skilled in the art, ring moieties described for one variable, e.g., Ring A, can also be applicable to other variables, e.g., Cy^{L}, if requirements of the other variables, e.g., number of heteroatoms, valence, etc., are satisfied. Example rings are extensively described in the present disclosure.

In some embodiments, a ring, which is optionally substituted, is a 3-20 membered monocyclic, bicyclic or polycyclic ring having 0-10 heteroatoms independently selected from oxygen, nitrogen, sulfur, phosphorus and silicon.

In some embodiments, a ring can be of any size within its range, e.g., 3, 4, 5, 6, 7, 8, 9, 10, 11, 12, 13, 14, 15, 16, 17, 18, 19, or 20-membered.

In some embodiments, a ring is monocyclic. In some embodiments, a ring is saturated and monocyclic. In some embodiments, a ring is monocyclic and partially saturated. In some embodiments, a ring is monocyclic and aromatic.

In some embodiments, a ring is bicyclic. In some embodiments, a ring is polycyclic. In some embodiments, a bicyclic or polycyclic ring comprises two or more monocyclic ring moieties, each of which can be saturated, partially saturated, or aromatic, and each which can contain no or 1-10 heteroatoms. In some embodiments, a bicyclic or polycyclic ring comprises a saturated monocyclic ring. In some embodiments, a bicyclic or polycyclic ring comprises a saturated monocyclic ring containing no heteroatoms. In some embodiments, a bicyclic or polycyclic ring comprises a saturated monocyclic ring comprising one or more heteroatoms. In some embodiments, a bicyclic or polycyclic ring comprises a partially saturated monocyclic ring. In some embodiments, a bicyclic or polycyclic ring comprises a partially saturated monocyclic ring containing no heteroatoms. In some embodiments, a bicyclic or polycyclic ring comprises a partially saturated monocyclic ring comprising one or more heteroatoms. In some embodiments, a bicyclic or polycyclic ring comprises an aromatic monocyclic ring. In some embodiments, a bicyclic or polycyclic ring comprises an aromatic monocyclic ring containing no heteroatoms. In some embodiments, a bicyclic or polycyclic ring comprises an aromatic monocyclic ring comprising one or more heteroatoms. In some embodiments, a bicyclic or polycyclic ring comprises a saturated ring and a partially saturated ring, each of which independently contains one or more heteroatoms. In some embodiments, a bicyclic ring comprises a saturated ring and a partially saturated ring, each of which independently comprises no, or one or more heteroatoms. In some embodiments, a bicyclic ring comprises an aromatic ring and a partially saturated ring, each of which independently comprises no, or one or more heteroatoms. In some embodiments, a polycyclic ring comprises a saturated ring and a partially saturated ring, each of which independently comprises no, or one or more heteroatoms. In some embodiments, a polycyclic ring comprises an aromatic ring and a partially saturated ring, each of which independently comprises no, or one or more heteroatoms. In some embodiments, a polycyclic ring comprises an aromatic ring and a saturated ring, each of which independently comprises no, or one or more heteroatoms. In some embodiments, a polycyclic ring comprises an aromatic ring, a saturated ring, and a partially saturated ring, each of which independently comprises no, or one or more heteroatoms. In some embodiments, a ring comprises at least one heteroatom. In some embodiments, a ring comprises at least one nitrogen atom. In some embodiments, a ring comprises at least one oxygen atom. In some embodiments, a ring comprises at least one sulfur atom.

As appreciated by those skilled in the art in accordance with the present disclosure, a ring is typically optionally substituted. In some embodiments, a ring is unsubstituted. In some embodiments, a ring is substituted. In some embodiments, a ring is substituted on one or more of its carbon atoms. In some embodiments, a ring is substituted on one or more of its heteroatoms. In some embodiments, a ring is substituted on one or more of its carbon atoms, and one or more of its heteroatoms. In some embodiments, two or more substituents can be located on the same ring atom. In some embodiments, all available ring atoms are substituted. In some embodiments, not all available ring atoms are substituted. In some embodiments, in provided structures where rings are indicated to be connected to other structures (e.g., Ring A in

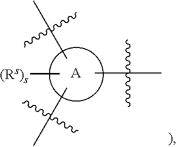

“optionally substituted” is to mean that, besides those structures already connected, remaining substitutable ring positions, if any, are optionally substituted.

In some embodiments, a ring is a bivalent or multivalent C₃₋₃₀ cycloaliphatic ring. In some embodiments, a ring is a bivalent or multivalent C₃₋₂₀ cycloaliphatic ring. In some embodiments, a ring is a bivalent or multivalent C₃₋₁₀ cycloaliphatic ring. In some embodiments, a ring is a bivalent or multivalent 3-30 membered saturated or partially unsaturated carbocyclic ring. In some embodiments, a ring is a bivalent or multivalent 3-7 membered saturated or partially unsaturated carbocyclic ring. In some embodiments, a ring is a bivalent or multivalent 3-membered saturated or partially unsaturated carbocyclic ring. In some embodiments, a ring is a bivalent or multivalent 4-membered saturated or partially unsaturated carbocyclic ring. In some embodiments, a ring is a bivalent or multivalent 5-membered saturated or partially unsaturated carbocyclic ring. In some embodiments, a ring is a bivalent or multivalent 6-membered saturated or partially unsaturated carbocyclic ring. In some embodiments, a ring is a bivalent or multivalent 7-membered saturated or partially unsaturated carbocyclic ring. In some embodiments, a ring is a bivalent or multivalent cyclohexyl ring. In some embodiments, a ring is a bivalent or multivalent cyclopentyl ring. In some embodiments, a ring is a bivalent or multivalent cyclobutyl ring. In some embodiments, a ring is a bivalent or multivalent cyclopropyl ring.

In some embodiments, a ring is a bivalent or multivalent C₆₋₃₀ aryl ring. In some embodiments, a ring is a bivalent or multivalent phenyl ring.

In some embodiments, a ring is a bivalent or multivalent 8-10 membered bicyclic saturated, partially unsaturated or aryl ring. In some embodiments, a ring is a bivalent or multivalent 8-10 membered bicyclic saturated ring. In some embodiments, a ring is a bivalent or multivalent 8-10 membered bicyclic partially unsaturated ring. In some embodiments, a ring is a bivalent or multivalent 8-10 membered bicyclic aryl ring. In some embodiments, a ring is a bivalent or multivalent naphthyl ring.

In some embodiments, a ring is a bivalent or multivalent 5-30 membered heteroaryl ring having 1-10 heteroatoms independently selected from oxygen, nitrogen, sulfur, phosphorus and silicon. In some embodiments, a ring is a bivalent or multivalent 5-30 membered heteroaryl ring having 1-10 heteroatoms independently selected from oxygen, nitrogen, and sulfur. In some embodiments, a ring is a bivalent or multivalent 5-30 membered heteroaryl ring having 1-5 heteroatoms independently selected from oxygen, nitrogen, sulfur, phosphorus and silicon. In some embodiments, a ring is a bivalent or multivalent 5-30 membered heteroaryl ring having 1-5 heteroatoms independently selected from oxygen, nitrogen, and sulfur.

In some embodiments, a ring is a bivalent or multivalent 5-6 membered monocyclic heteroaryl ring having 1-4 heteroatoms independently selected from nitrogen, oxygen, and sulfur. In some embodiments, a ring is a bivalent or multivalent 5-6 membered monocyclic heteroaryl ring having 1-3 heteroatoms independently selected from nitrogen, sulfur, and oxygen.

In some embodiments, a ring is a bivalent or multivalent 5-membered monocyclic heteroaryl ring having 1-4 heteroatoms independently selected from nitrogen, oxygen or sulfur. In some embodiments, a ring is a bivalent or multivalent 6-membered monocyclic heteroaryl ring having 1-4 heteroatoms independently selected from nitrogen, oxygen, and sulfur.

In certain embodiments, a ring is a bivalent or multivalent 8-10 membered bicyclic heteroaryl ring having 1-4 heteroatoms independently selected from nitrogen, oxygen, and sulfur. In some embodiments, a ring is a bivalent or multivalent 5,6-fused heteroaryl ring having 1-4 heteroatoms independently selected from nitrogen, oxygen, and sulfur. In some embodiments, a ring is a bivalent or multivalent 5,6-fused heteroaryl ring having 1-5 heteroatoms independently selected from nitrogen, oxygen, and sulfur. In certain embodiments, a ring is a bivalent or multivalent 6,6-fused heteroaryl ring having 1-4 heteroatoms independently selected from nitrogen, oxygen, and sulfur.

In some embodiments, a ring is a bivalent or multivalent 3-30 membered heterocyclic ring having 1-10 heteroatoms independently selected from oxygen, nitrogen, sulfur, phosphorus and silicon. In some embodiments, a ring is a bivalent or multivalent 3-7 membered saturated or partially unsaturated heterocyclic ring having 1-3 heteroatoms independently selected from nitrogen, oxygen, and sulfur. In certain embodiments, a ring is a bivalent or multivalent 5-7 membered partially unsaturated monocyclic ring having 1-3 heteroatoms independently selected from nitrogen, oxygen, and sulfur. In certain embodiments, a ring is a bivalent or multivalent 5-6 membered partially unsaturated monocyclic ring having 1-3 heteroatoms independently selected from nitrogen, oxygen, and sulfur. In certain embodiments, a ring is a bivalent or multivalent 5-membered partially unsaturated monocyclic ring having 1-3 heteroatoms independently selected from nitrogen, oxygen, and sulfur. In certain embodiments, a ring is a bivalent or multivalent 6-membered partially unsaturated monocyclic ring having 1-3 heteroatoms independently selected from nitrogen, oxygen, and sulfur. In certain embodiments, a ring is a bivalent or multivalent 7-membered partially unsaturated monocyclic ring having 1-3 heteroatoms independently selected from nitrogen, oxygen, and sulfur. In some embodiments, a ring is a bivalent or multivalent 3-membered heterocyclic ring having one heteroatom selected from nitrogen, oxygen or sulfur. In some embodiments, a ring is a bivalent or multivalent 4-membered heterocyclic ring having 1-3 heteroatoms independently selected from nitrogen, oxygen, and sulfur. In some embodiments, a ring is a bivalent or multivalent 5-membered heterocyclic ring having 1-3 heteroatoms independently selected from nitrogen, oxygen, and sulfur. In some embodiments, a ring is a bivalent or multivalent 6-membered heterocyclic ring having 1-3 heteroatoms independently selected from nitrogen, oxygen, and sulfur. In some embodiments, a ring is a bivalent or multivalent 7-membered heterocyclic ring having 1-3 heteroatoms independently selected from nitrogen, oxygen, and sulfur.

In some embodiments, a ring is a bivalent or multivalent 7-10 membered bicyclic saturated or partially unsaturated heterocyclic ring having 1-5 heteroatoms independently selected from nitrogen, oxygen, and sulfur. In some embodiments, a ring is a bivalent or multivalent 8-10 membered bicyclic heteroaryl ring having 1-5 heteroatoms independently selected from nitrogen, oxygen, and sulfur.

In some embodiments, a ring is a bivalent or multivalent 5,6-fused heteroaryl ring having 1-5 heteroatoms independently selected from nitrogen, oxygen, and sulfur. In certain embodiments, a ring is a bivalent or multivalent 6,6-fused heteroaryl ring having 1-5 heteroatoms independently selected from nitrogen, oxygen, and sulfur.

In some embodiments, a ring formed by two or more groups taken together, which is typically optionally substituted, is a monocyclic saturated 5-7 membered ring having no additional heteroatoms in addition to intervening heteroatoms, if any. In some embodiments, a ring formed by two or more groups taken together is a monocyclic saturated 5-membered ring having no additional heteroatoms in addition to intervening heteroatoms, if any. In some embodiments, a ring formed by two or more groups taken together is a monocyclic saturated 6-membered ring having no additional heteroatoms in addition to intervening heteroatoms, if any. In some embodiments, a ring formed by two or more groups taken together is a monocyclic saturated 7-membered ring having no additional heteroatoms in addition to intervening heteroatoms, if any.

In some embodiments, a ring formed by two or more groups taken together is a bicyclic, saturated, partially unsaturated, or aryl 5-30 membered ring having, in addition to the intervening heteroatoms, if any, 0-10 heteroatoms independently selected from oxygen, nitrogen, sulfur, phosphorus and silicon. In some embodiments, a ring formed by two or more groups taken together is a bicyclic, saturated, partially unsaturated, or aryl 5-30 membered ring having, in addition to the intervening heteroatoms, if any, 0-10 heteroatoms independently selected from oxygen, nitrogen, and sulfur. In some embodiments, a ring formed by two or more groups taken together is a bicyclic and saturated 8-10 membered bicyclic ring having no additional heteroatoms in addition to intervening heteroatoms, if any. In some embodiments, a ring formed by two or more groups taken together is a bicyclic and saturated 8-membered bicyclic ring having no additional heteroatoms in addition to intervening heteroatoms, if any. In some embodiments, a ring formed by two or more groups taken together is a bicyclic and saturated 9-membered bicyclic ring having no additional heteroatoms in addition to intervening heteroatoms, if any. In some embodiments, a ring formed by two or more groups taken together is a bicyclic and saturated 10-membered bicyclic ring having no additional heteroatoms in addition to intervening heteroatoms, if any. In some embodiments, a ring formed by two or more groups taken together is bicyclic and comprises a 5-membered ring fused to a 5-membered ring. In some embodiments, a ring formed by two or more groups taken together is bicyclic and comprises a 5-membered ring fused to a 6-membered ring. In some embodiments, the 5-membered ring comprises one or more intervening nitrogen, phosphorus and oxygen atoms as ring atoms. In some embodiments, a ring formed by two or more groups taken together comprises a ring system having the backbone structure of

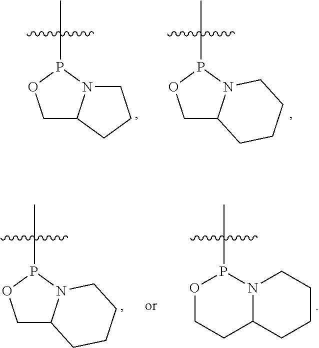 [SMILES: *P1OCC2CCCN21 ; *P1OCC2CCCCN21 ; *P1OCC2CCCCN21 ; *P1OCCC2CCCCN21]

In some embodiments, a ring formed by two or more groups taken together is a polycyclic, saturated, partially unsaturated, or aryl 3-30 membered ring having, in addition to the intervening heteroatoms, if any, 0-10 heteroatoms independently selected from oxygen, nitrogen, sulfur, phosphorus and silicon. In some embodiments, a ring formed by two or more groups taken together is a polycyclic, saturated, partially unsaturated, or aryl 3-30 membered ring having, in addition to the intervening heteroatoms, if any, 0-10 heteroatoms independently selected from oxygen, nitrogen, and sulfur.

In some embodiments, a ring formed by two or more groups taken together is monocyclic, bicyclic or polycyclic and comprises a 5-10 membered monocyclic ring whose ring atoms comprise one or more intervening nitrogen, phosphorus and/or oxygen atoms. In some embodiments, a ring formed by two or more groups taken together is monocyclic, bicyclic or polycyclic and comprises a 5-9 membered monocyclic ring whose ring atoms comprise one or more intervening nitrogen, phosphorus and/or oxygen atoms. In some embodiments, a ring formed by two or more groups taken together is monocyclic, bicyclic or polycyclic and comprises a 5-8 membered monocyclic ring whose ring atoms comprise one or more intervening nitrogen, phosphorus and/or oxygen atoms. In some embodiments, a ring formed by two or more groups taken together is monocyclic, bicyclic or polycyclic and comprises a 5-7 membered monocyclic ring whose ring atoms comprise one or more intervening nitrogen, phosphorus and/or oxygen atoms. In some embodiments, a ring formed by two or more groups taken together is monocyclic, bicyclic or polycyclic and comprises a 5-6 membered monocyclic ring whose ring atoms comprise one or more intervening nitrogen, phosphorus and/or oxygen atoms.

In some embodiments, a ring formed by two or more groups taken together is monocyclic, bicyclic or polycyclic and comprises a 5-membered monocyclic ring whose ring atoms comprise one or more intervening nitrogen, phosphorus and/or oxygen atoms. In some embodiments, a ring formed by two or more groups taken together is monocyclic, bicyclic or polycyclic and comprises a 6-membered monocyclic ring whose ring atoms comprise one or more intervening nitrogen, phosphorus and/or oxygen atoms. In some embodiments, a ring formed by two or more groups taken together is monocyclic, bicyclic or polycyclic and comprises a 7-membered monocyclic ring whose ring atoms comprise one or more intervening nitrogen, phosphorus and/or oxygen atoms. In some embodiments, a ring formed by two or more groups taken together is monocyclic, bicyclic or polycyclic and comprises a 8-membered monocyclic ring whose ring atoms comprise one or more intervening nitrogen, phosphorus and/or oxygen atoms. In some embodiments, a ring formed by two or more groups taken together is monocyclic, bicyclic or polycyclic and comprises a 9-membered monocyclic ring whose ring atoms comprise one or more intervening nitrogen, phosphorus and/or oxygen atoms. In some embodiments, a ring formed by two or more groups taken together is monocyclic, bicyclic or polycyclic and comprises a 10-membered monocyclic ring whose ring atoms comprise one or more intervening nitrogen, phosphorus and/or oxygen atoms.

In some embodiments, a ring formed by two or more groups taken together is monocyclic, bicyclic or polycyclic and comprises a 5-membered ring whose ring atoms consist of carbon atoms and the intervening nitrogen, phosphorus and oxygen atoms. In some embodiments, a ring formed by two or more groups taken together is monocyclic, bicyclic or polycyclic and comprises a 6-membered ring whose ring atoms consist of carbon atoms and the intervening nitrogen, phosphorus and oxygen atoms. In some embodiments, a ring formed by two or more groups taken together is monocyclic, bicyclic or polycyclic and comprises a 7-membered ring whose ring atoms consist of carbon atoms and the intervening nitrogen, phosphorus and oxygen atoms. In some embodiments, a ring formed by two or more groups taken together is monocyclic, bicyclic or polycyclic and comprises a 8-membered ring whose ring atoms consist of carbon atoms and the intervening nitrogen, phosphorus and oxygen atoms. In some embodiments, a ring formed by two or more groups taken together is monocyclic, bicyclic or polycyclic and comprises a 9-membered ring whose ring atoms consist of carbon atoms and the intervening nitrogen, phosphorus and oxygen atoms. In some embodiments, a ring formed by two or more groups taken together is monocyclic, bicyclic or polycyclic and comprises a 10-membered ring whose ring atoms consist of carbon atoms and the intervening nitrogen, phosphorus and oxygen atoms.

In some embodiments, rings described herein are unsubstituted. In some embodiments, rings described herein are substituted. In some embodiments, substituents are selected from those described in example compounds provided in the present disclosure.

As described herein, each LP is independently an internucleotidic linkage as described in the present disclosure, e.g., a natural phosphate linkage, a phosphorothioate diester linkage, a modified internucleotidic linkage, a chiral internucleotidic linkage, a non-negatively charged internucleotidic linkage, etc., In some embodiments, each LP is independently a linkage having the structure of formula I. In some embodiments, one or more LP independently have the structure of formula I, I-a-1, I-a-2, I-b, I-c, I-d, I-e, I-n-1, I-n-2, I-n-3, I-n-4, II, II-a-1, II-a-2, II-b-1, II-b-2, II-c-1, II-c-2, II-d-1, or II-d-2, or a salt form thereof. In some embodiments, at least one LP is a non-negatively charged internucleotidic linkage. In some embodiments, at least one LP is a neutral internucleotidic linkage. In some embodiments, one or more LP independently have the structure of formula I-n-1, I-n-2, I-n-3, I-n-4, II, II-a-1, II-a-2, II-b-1, II-b-2, II-c-1, II-c-2, II-d-1, or II-d-2, or a salt form thereof.

In some embodiments, L^{3E} is -L^{s}- or -L^{s}-L^{s}-. In some embodiments, L^{3E} is -L^{s}-. In some embodiments, L^{3E} is -L^{s}-L^{s}-. In some embodiments, L^{3E} is a covalent bond. In some embodiments, L^{3E} is a linker used in oligonucleotide synthesis. In some embodiments, L^{3E} is a linker used in solid phase oligonucleotide synthesis. Various types of linkers are known and can be utilized in accordance with the present disclosure. In some embodiments, a linker is a succinate linker (—O—C(O)—CH₂—CH₂—C(O)—). In some embodiments, a linker is an oxalyl linker (—O—C(O)—C(O)—). In some embodiments, L^{3E} is a succinyl-piperidine linker (SP) linker. In some embodiments, L^{3E} is a succinyl linker. In some embodiments, L^{3E} is a Q-linker.

In some embodiments, R^{3E} is —R′, -L^{s}-R′, —OR′, or a solid support. In some embodiments, R^{3E} is —R′. In some embodiments, R^{3E} i-L^{s}-R′. In some embodiments, R^{3E} is —OR′. In some embodiments, R^{3E} is a support for oligonucleotide synthesis. In some embodiments, R^{3E} is a solid support. In some embodiments, a solid support is a CPG support. In some embodiments, a solid support is a polystyrene support. In some embodiments, R^{3E} is —H. In some embodiments, -L³-R^{3E} is —H. In some embodiments, R^{3E} is —OH. In some embodiments, -L³-R^{3E} is —OH. In some embodiments, R^{3E} is optionally substituted C₁₋₆ aliphatic. In some embodiments, R^{3E} is optionally substituted C₁₋₆ alkyl. In some embodiments, R^{3E} is —OR′. In some embodiments, R^{3E} is —OH. In some embodiments, R^{3E} is —OR′, wherein R′ is not hydrogen. In some embodiments, R^{3E} is —OR′, wherein R′ is optionally substituted C₁₋₆ alkyl. In some embodiments, R^{3E} is a 3′-end cap (e.g., those used in RNAi technologies).

In some embodiments, R^{3E} is a solid support. In some embodiments, R^{3E} is a solid support for oligonucleotide synthesis. Various types of solid support are known and can be utilized in accordance with the present disclosure. In some embodiments, a solid support is HCP. In some embodiments, a solid support is CPG.

In some embodiments, R′ is —R, —C(O)R, —C(O)OR, or —S(O)₂R, wherein R is as described in the present disclosure. In some embodiments, R′ is R, wherein R is as described in the present disclosure. In some embodiments, R′ is —C(O)R, wherein R is as described in the present disclosure. In some embodiments, R′ is —C(O)OR, wherein R is as described in the present disclosure. In some embodiments, R′ is —S(O)₂R, wherein R is as described in the present disclosure. In some embodiments, R′ is hydrogen. In some embodiments, R′ is not hydrogen. In some embodiments, R′ is R, wherein R is optionally substituted C₁₋₂₀ aliphatic as described in the present disclosure. In some embodiments, R′ is R, wherein R is optionally substituted C₁₋₂₀ heteroaliphatic as described in the present disclosure. In some embodiments, R′ is R, wherein R is optionally substituted C₆₋₂₀ aryl as described in the present disclosure. In some embodiments, R′ is R, wherein R is optionally substituted C₆₋₂₀ arylaliphatic as described in the present disclosure. In some embodiments, R′ is R, wherein R is optionally substituted C₆₋₂₀ arylheteroaliphatic as described in the present disclosure. In some embodiments, R′ is R, wherein R is optionally substituted 5-20 membered heteroaryl as described in the present disclosure. In some embodiments, R′ is R, wherein R is optionally substituted 3-20 membered heterocyclyl as described in the present disclosure. In some embodiments, two or more R′ are R, and are optionally and independently taken together to form an optionally substituted ring as described in the present disclosure.

In some embodiments, each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or.
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon.
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, each R is independently —H, or an optionally substituted group selected from C₁₋₂₀ aliphatic, C₁₋₂₀ heteroaliphatic having 1-10 heteroatoms independently selected from oxygen, nitrogen, sulfur, phosphorus and silicon, C₆₋₂₀ aryl, C₆₋₂₀ arylaliphatic, C₆₋₂₀ arylheteroaliphatic having 1-10 heteroatoms independently selected from oxygen, nitrogen, sulfur, phosphorus and silicon, 5-20 membered heteroaryl having 1-10 heteroatoms independently selected from oxygen, nitrogen, sulfur, phosphorus and silicon, and 3-20 membered heterocyclyl having 1-10 heteroatoms independently selected from oxygen, nitrogen, sulfur, phosphorus and silicon, or
two R groups are optionally and independently taken together to form a covalent bond, or.
two or more R groups on the same atom are optionally and independently taken together with the atom to form an optionally substituted, 3-20 membered monocyclic, bicyclic or polycyclic ring having, in addition to the atom, 0-10 heteroatoms independently selected from oxygen, nitrogen, sulfur, phosphorus and silicon.
two or more R groups on two or more atoms are optionally and independently taken together with their intervening atoms to form an optionally substituted, 3-20 membered monocyclic, bicyclic or polycyclic ring having, in addition to the intervening atoms, 0-10 heteroatoms independently selected from oxygen, nitrogen, sulfur, phosphorus and silicon.

In some embodiments, each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon.

In some embodiments, each R is independently —H, or an optionally substituted group selected from C₁₋₂₀ aliphatic, C₁₋₂₀ heteroaliphatic having 1-10 heteroatoms independently selected from oxygen, nitrogen, sulfur, phosphorus and silicon, C₆₋₂₀ aryl, C₆₋₂₀ arylaliphatic, C₆₋₂₀ arylheteroaliphatic having 1-10 heteroatoms independently selected from oxygen, nitrogen, sulfur, phosphorus and silicon, 5-20 membered heteroaryl having 1-10 heteroatoms independently selected from oxygen, nitrogen, sulfur, phosphorus and silicon, and 3-20 membered heterocyclyl having 1-10 heteroatoms independently selected from oxygen, nitrogen, sulfur, phosphorus and silicon.

In some embodiments, R is hydrogen. In some embodiments, R is not hydrogen. In some embodiments, R is an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, a 5-30 membered heteroaryl ring having 1-10 heteroatoms independently selected from oxygen, nitrogen, sulfur, phosphorus and silicon, and a 3-30 membered heterocyclic ring having 1-10 heteroatoms independently selected from oxygen, nitrogen, sulfur, phosphorus and silicon.

In some embodiments, R is hydrogen or an optionally substituted group selected from C₁₋₂₀ aliphatic, phenyl, a 3-7 membered saturated or partially unsaturated carbocyclic ring, an 8-10 membered bicyclic saturated, partially unsaturated or aryl ring, a 5-6 membered monocyclic heteroaryl ring having 1-4 heteroatoms independently selected from nitrogen, oxygen, and sulfur, a 4-7 membered saturated or partially unsaturated heterocyclic ring having 1-3 heteroatoms independently selected from nitrogen, oxygen, and sulfur, a 7-10 membered bicyclic saturated or partially unsaturated heterocyclic ring having 1-5 heteroatoms independently selected from nitrogen, oxygen, and sulfur, or an 8-10 membered bicyclic heteroaryl ring having 1-5 heteroatoms independently selected from nitrogen, oxygen, and sulfur.

In some embodiments, R is optionally substituted C₁₋₃₀ aliphatic. In some embodiments, R is optionally substituted C₁₋₂₀ aliphatic. In some embodiments, R is optionally substituted C₁₋₁₅ aliphatic. In some embodiments, R is optionally substituted C₁₋₁₀ aliphatic. In some embodiments, R is optionally substituted C₁₋₆ aliphatic. In some embodiments, R is optionally substituted C₁₋₆ alkyl. In some embodiments, R is optionally substituted hexyl, pentyl, butyl, propyl, ethyl or methyl. In some embodiments, R is optionally substituted hexyl. In some embodiments, R is optionally substituted pentyl. In some embodiments, R is optionally substituted butyl. In some embodiments, R is optionally substituted propyl. In some embodiments, R is optionally substituted ethyl. In some embodiments, R is optionally substituted methyl. In some embodiments, R is hexyl. In some embodiments, R is pentyl. In some embodiments, R is butyl. In some embodiments, R is propyl. In some embodiments, R is ethyl. In some embodiments, R is methyl. In some embodiments, R is isopropyl. In some embodiments, R is n-propyl. In some embodiments, R is tert-butyl. In some embodiments, R is sec-butyl. In some embodiments, R is n-butyl. In some embodiments, R is —(CH₂)₂CN.

In some embodiments, R is optionally substituted C₃₋₃₀ cycloaliphatic. In some embodiments, R is optionally substituted C₃₋₂₀ cycloaliphatic. In some embodiments, R is optionally substituted C₃₋₁₀ cycloaliphatic. In some embodiments, R is optionally substituted cyclohexyl. In some embodiments, R is cyclohexyl. In some embodiments, R is optionally substituted cyclopentyl. In some embodiments, R is cyclopentyl. In some embodiments, R is optionally substituted cyclobutyl. In some embodiments, R is cyclobutyl. In some embodiments, R is optionally substituted cyclopropyl. In some embodiments, R is cyclopropyl.

In some embodiments, R is an optionally substituted 3-30 membered saturated or partially unsaturated carbocyclic ring. In some embodiments, R is an optionally substituted 3-7 membered saturated or partially unsaturated carbocyclic ring. In some embodiments, R is an optionally substituted 3-membered saturated or partially unsaturated carbocyclic ring. In some embodiments, R is an optionally substituted 4-membered saturated or partially unsaturated carbocyclic ring. In some embodiments, R is an optionally substituted 5-membered saturated or partially unsaturated carbocyclic ring. In some embodiments, R is an optionally substituted 6-membered saturated or partially unsaturated carbocyclic ring. In some embodiments, R is an optionally substituted 7-membered saturated or partially unsaturated carbocyclic ring. In some embodiments, R is optionally substituted cycloheptyl. In some embodiments, R is cycloheptyl. In some embodiments, R is optionally substituted cyclohexyl. In some embodiments, R is cyclohexyl. In some embodiments, R is optionally substituted cyclopentyl. In some embodiments, R is cyclopentyl. In some embodiments, R is optionally substituted cyclobutyl. In some embodiments, R is cyclobutyl. In some embodiments, R is optionally substituted cyclopropyl. In some embodiments, R is cyclopropyl.

In some embodiments, when R is or comprises a ring structure, e.g., cycloaliphatic, cycloheteroaliphatic, aryl, heteroaryl, etc., the ring structure can be monocyclic, bicyclic or polycyclic. In some embodiments, R is or comprises a monocyclic structure. In some embodiments, R is or comprises a bicyclic structure. In some embodiments, R is or comprises a polycyclic structure.

In some embodiments, R is optionally substituted C₁₋₃₀ heteroaliphatic having 1-10 heteroatoms independently selected from oxygen, nitrogen, sulfur, phosphorus and silicon. In some embodiments, R is optionally substituted C₁₋₂₀ heteroaliphatic having 1-10 heteroatoms. In some embodiments, R is optionally substituted C₁₋₂₀ heteroaliphatic having 1-10 heteroatoms independently selected from oxygen, nitrogen, sulfur, phosphorus or silicon, optionally including one or more oxidized forms of nitrogen, sulfur, phosphorus or selenium. In some embodiments, R is optionally substituted C₁₋₃₀ heteroaliphatic comprising 1-10 groups independently selected from

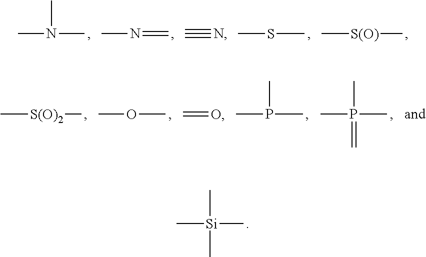

In some embodiments, R is optionally substituted C₆₋₃₀ aryl. In some embodiments, R is optionally substituted phenyl. In some embodiments, R is phenyl. In some embodiments, R is substituted phenyl.

In some embodiments, R is an optionally substituted 8-10 membered bicyclic saturated, partially unsaturated or aryl ring. In some embodiments, R is an optionally substituted 8-10 membered bicyclic saturated ring. In some embodiments, R is an optionally substituted 8-10 membered bicyclic partially unsaturated ring. In some embodiments, R is an optionally substituted 8-10 membered bicyclic aryl ring. In some embodiments, R is optionally substituted naphthyl.

In some embodiments, R is optionally substituted 5-30 membered heteroaryl ring having 1-10 heteroatoms independently selected from oxygen, nitrogen, sulfur, phosphorus and silicon. In some embodiments, R is optionally substituted 5-30 membered heteroaryl ring having 1-10 heteroatoms independently selected from oxygen, nitrogen, and sulfur. In some embodiments, R is optionally substituted 5-30 membered heteroaryl ring having 1-5 heteroatoms independently selected from oxygen, nitrogen, sulfur, phosphorus and silicon. In some embodiments, R is optionally substituted 5-30 membered heteroaryl ring having 1-5 heteroatoms independently selected from oxygen, nitrogen, and sulfur.

In some embodiments, R is an optionally substituted 5-6 membered monocyclic heteroaryl ring having 1-4 heteroatoms independently selected from nitrogen, oxygen, and sulfur. In some embodiments, R is a substituted 5-6 membered monocyclic heteroaryl ring having 1-4 heteroatoms independently selected from nitrogen, oxygen, and sulfur. In some embodiments, R is an unsubstituted 5-6 membered monocyclic heteroaryl ring having 1-4 heteroatoms independently selected from nitrogen, oxygen, and sulfur. In some embodiments, R is an optionally substituted 5-6 membered monocyclic heteroaryl ring having 1-3 heteroatoms independently selected from nitrogen, sulfur, and oxygen. In some embodiments, R is a substituted 5-6 membered monocyclic heteroaryl ring having 1-3 heteroatoms independently selected from nitrogen, oxygen, and sulfur. In some embodiments, R is an unsubstituted 5-6 membered monocyclic heteroaryl ring having 1-3 heteroatoms independently selected from nitrogen, sulfur, and oxygen.

In some embodiments, R is an optionally substituted 5-membered monocyclic heteroaryl ring having 1-4 heteroatoms independently selected from nitrogen, oxygen or sulfur. In some embodiments, R is an optionally substituted 6-membered monocyclic heteroaryl ring having 1-4 heteroatoms independently selected from nitrogen, oxygen, and sulfur.

In some embodiments, R is an optionally substituted 5-membered monocyclic heteroaryl ring having one heteroatom selected from nitrogen, oxygen, and sulfur. In some embodiments, R is selected from optionally substituted pyrrolyl, furanyl, or thienyl.

In some embodiments, R is an optionally substituted 5-membered heteroaryl ring having two heteroatoms independently selected from nitrogen, oxygen, and sulfur. In certain embodiments, R is an optionally substituted 5-membered heteroaryl ring having one nitrogen atom, and an additional heteroatom selected from sulfur or oxygen. Example R groups include but are not limited to optionally substituted pyrazolyl, imidazolyl, thiazolyl, isothiazolyl, oxazolyl or isoxazolyl.

In some embodiments, R is an optionally substituted 5-membered heteroaryl ring having three heteroatoms independently selected from nitrogen, oxygen, and sulfur. Example R groups include but are not limited to optionally substituted triazolyl, oxadiazolyl or thiadiazolyl.

In some embodiments, R is an optionally substituted 5-membered heteroaryl ring having four heteroatoms independently selected from nitrogen, oxygen, and sulfur. Example R groups include but are not limited to optionally substituted tetrazolyl, oxatriazolyl and thiatriazolyl.

In some embodiments, R is an optionally substituted 6-membered heteroaryl ring having 1-4 nitrogen atoms. In some embodiments, R is an optionally substituted 6-membered heteroaryl ring having 1-3 nitrogen atoms. In other embodiments, R is an optionally substituted 6-membered heteroaryl ring having 1-2 nitrogen atoms. In some embodiments, R is an optionally substituted 6-membered heteroaryl ring having four nitrogen atoms. In some embodiments, R is an optionally substituted 6-membered heteroaryl ring having three nitrogen atoms. In some embodiments, R is an optionally substituted 6-membered heteroaryl ring having two nitrogen atoms. In certain embodiments, R is an optionally substituted 6-membered heteroaryl ring having one nitrogen atom. Example R groups include but are not limited to optionally substituted pyridinyl, pyrimidinyl, pyrazinyl, pyridazinyl, triazinyl, or tetrazinyl.

In certain embodiments, R is an optionally substituted 8-10 membered bicyclic heteroaryl ring having 1-4 heteroatoms independently selected from nitrogen, oxygen, and sulfur. In some embodiments, R is an optionally substituted 5,6-fused heteroaryl ring having 1-4 heteroatoms independently selected from nitrogen, oxygen, and sulfur. In other embodiments, R is an optionally substituted 5,6-fused heteroaryl ring having 1-2 heteroatoms independently selected from nitrogen, oxygen, and sulfur. In certain embodiments, R is an optionally substituted 5,6-fused heteroaryl ring having 1 heteroatom independently selected from nitrogen, oxygen, and sulfur. In some embodiments, R is an optionally substituted indolyl. In some embodiments, R is an optionally substituted azabicyclo[3.2.1]octanyl. In certain embodiments, R is an optionally substituted 5,6-fused heteroaryl ring having 2 heteroatoms independently selected from nitrogen, oxygen, and sulfur. In some embodiments, R is an optionally substituted azaindolyl. In some embodiments, R is an optionally substituted benzimidazolyl. In some embodiments, R is an optionally substituted benzothiazolyl. In some embodiments, R is an optionally substituted benzoxazolyl. In some embodiments, R is an optionally substituted indazolyl. In certain embodiments, R is an optionally substituted 5,6-fused heteroaryl ring having 3 heteroatoms independently selected from nitrogen, oxygen, and sulfur.

In some embodiments, R is an optionally substituted 5,6-fused heteroaryl ring having 1-5 heteroatoms independently selected from nitrogen, oxygen, and sulfur. In some embodiments, R is an optionally substituted 5,6-fused heteroaryl ring having 1-4 heteroatoms independently selected from nitrogen, oxygen, and sulfur. In some embodiments, R is an optionally substituted 5,6-fused heteroaryl ring having 1-3 heteroatoms independently selected from nitrogen, oxygen, and sulfur. In some embodiments, R is an optionally substituted 5,6-fused heteroaryl ring having two heteroatoms independently selected from nitrogen, oxygen, and sulfur. In some embodiments, R is an optionally substituted 5,6-fused heteroaryl ring having three heteroatoms independently selected from nitrogen, oxygen, and sulfur. In some embodiments, R is an optionally substituted 5,6-fused heteroaryl ring having four heteroatoms independently selected from nitrogen, oxygen, and sulfur. In some embodiments, R is an optionally substituted 5,6-fused heteroaryl ring having five heteroatoms independently selected from nitrogen, oxygen, and sulfur.

In certain embodiments, R is an optionally substituted 5,6-fused heteroaryl ring having one heteroatom independently selected from nitrogen, oxygen, and sulfur. In some embodiments, R is optionally substituted indolyl. In some embodiments, R is optionally substituted benzofuranyl. In some embodiments, R is optionally substituted benzo[b]thienyl. In certain embodiments, R is an optionally substituted 5,6-fused heteroaryl ring having two heteroatoms independently selected from nitrogen, oxygen, and sulfur. In some embodiments, R is optionally substituted azaindolyl. In some embodiments, R is optionally substituted benzimidazolyl. In some embodiments, R is optionally substituted benzothiazolyl. In some embodiments, R is optionally substituted benzoxazolyl. In some embodiments, R is an optionally substituted indazolyl. In certain embodiments, R is an optionally substituted 5,6-fused heteroaryl ring having three heteroatoms independently selected from nitrogen, oxygen, and sulfur. In some embodiments, R is optionally substituted oxazolopyridiyl, thiazolopyridinyl or imidazopyridinyl. In certain embodiments, R is an optionally substituted 5,6-fused heteroaryl ring having four heteroatoms independently selected from nitrogen, oxygen, and sulfur. In some embodiments, R is optionally substituted purinyl, oxazolopyrimidinyl, thiazolopyrimidinyl, oxazolopyrazinyl, thiazolopyrazinyl, imidazopyrazinyl, oxazolopyridazinyl, thiazolopyridazinyl or imidazopyridazinyl. In certain embodiments, R is an optionally substituted 5,6-fused heteroaryl ring having five heteroatoms independently selected from nitrogen, oxygen, and sulfur.

In some embodiments, R is optionally substituted 1,4-dihydropyrrolo[3,2-b]pyrrolyl, 4H-furo[3,2-b]pyrrolyl, 4H-thieno[3,2-b]pyrrolyl, furo[3,2-b]furanyl, thieno[3,2-b]furanyl, thieno[3,2-b]thienyl, 1H-pyrrolo[1,2-a]imidazolyl, pyrrolo[2,1-b]oxazolyl or pyrrolo[2,1-b]thiazolyl. In some embodiments, R is optionally substituted dihydropyrroloimidazolyl, 1H-furoimidazolyl, 1H-thienoimidazolyl, furooxazolyl, furoisoxazolyl, 4H-pyrrolooxazolyl, 4H-pyrroloisoxazolyl, thienooxazolyl, thienoisoxazolyl, 4H-pyrrolothiazolyl, furothiazolyl, thienothiazolyl, 1H-imidazoimidazolyl, imidazooxazolyl or imidazo[5,1-b]thiazolyl.

In certain embodiments, R is an optionally substituted 6,6-fused heteroaryl ring having 1-4 heteroatoms independently selected from nitrogen, oxygen, and sulfur. In some embodiments, R is an optionally substituted 6,6-fused heteroaryl ring having 1-2 heteroatoms independently selected from nitrogen, oxygen, and sulfur. In other embodiments, R is an optionally substituted 6,6-fused heteroaryl ring having 1 heteroatom independently selected from nitrogen, oxygen, and sulfur. In some embodiments, R is an optionally substituted quinolinyl. In some embodiments, R is an optionally substituted isoquinolinyl. In some embodiments, R is an optionally substituted 6,6-fused heteroaryl ring having 2 heteroatoms independently selected from nitrogen, oxygen, and sulfur. In some embodiments, R is optionally substituted quinazoline or a quinoxaline.

In some embodiments, R is 3-30 membered heterocyclic ring having 1-10 heteroatoms independently selected from oxygen, nitrogen, sulfur, phosphorus and silicon. In some embodiments, R is 3-30 membered heterocyclic ring having 1-10 heteroatoms independently selected from oxygen, nitrogen, and sulfur. In some embodiments, R is 3-30 membered heterocyclic ring having 1-5 heteroatoms independently selected from oxygen, nitrogen, sulfur, phosphorus and silicon. In some embodiments, R is 3-30 membered heterocyclic ring having 1-5 heteroatoms independently selected from oxygen, nitrogen, and sulfur.

In some embodiments, R is an optionally substituted 3-7 membered saturated or partially unsaturated heterocyclic ring having 1-3 heteroatoms independently selected from nitrogen, oxygen, and sulfur. In some embodiments, R is a substituted 3-7 membered saturated or partially unsaturated heterocyclic ring having 1-3 heteroatoms independently selected from nitrogen, oxygen, and sulfur. In some embodiments, R is an unsubstituted 3-7 membered saturated or partially unsaturated heterocyclic ring having 1-3 heteroatoms independently selected from nitrogen, oxygen, and sulfur. In certain embodiments, R is an optionally substituted 5-7 membered partially unsaturated monocyclic ring having 1-3 heteroatoms independently selected from nitrogen, oxygen, and sulfur. In certain embodiments, R is an optionally substituted 5-6 membered partially unsaturated monocyclic ring having 1-3 heteroatoms independently selected from nitrogen, oxygen, and sulfur. In certain embodiments, R is an optionally substituted 5-membered partially unsaturated monocyclic ring having 1-3 heteroatoms independently selected from nitrogen, oxygen, and sulfur. In certain embodiments, R is an optionally substituted 6-membered partially unsaturated monocyclic ring having 1-3 heteroatoms independently selected from nitrogen, oxygen, and sulfur. In certain embodiments, R is an optionally substituted 7-membered partially unsaturated monocyclic ring having 1-3 heteroatoms independently selected from nitrogen, oxygen, and sulfur. In some embodiments, R is optionally substituted 3-membered heterocyclic ring having one heteroatom selected from nitrogen, oxygen or sulfur. In some embodiments, R is optionally substituted 4-membered heterocyclic ring having 1-3 heteroatoms independently selected from nitrogen, oxygen, and sulfur. In some embodiments, R is optionally substituted 5-membered heterocyclic ring having 1-3 heteroatoms independently selected from nitrogen, oxygen, and sulfur. In some embodiments, R is optionally substituted 6-membered heterocyclic ring having 1-3 heteroatoms independently selected from nitrogen, oxygen, and sulfur. In some embodiments, R is optionally substituted 7-membered heterocyclic ring having 1-3 heteroatoms independently selected from nitrogen, oxygen, and sulfur.

In some embodiments, R is an optionally substituted 3-membered saturated or partially unsaturated heterocyclic ring having 1-2 heteroatoms independently selected from nitrogen, oxygen, and sulfur. In some embodiments, R is an optionally substituted 4-membered saturated or partially unsaturated heterocyclic ring having 1-2 heteroatoms independently selected from nitrogen, oxygen, and sulfur. In some embodiments, R is an optionally substituted 5-membered saturated or partially unsaturated heterocyclic ring having 1-2 heteroatoms independently selected from nitrogen, oxygen, and sulfur. In some embodiments, R is an optionally substituted 6-membered saturated or partially unsaturated heterocyclic ring having 1-2 heteroatoms independently selected from nitrogen, oxygen, and sulfur. In some embodiments, R is an optionally substituted 7-membered saturated or partially unsaturated heterocyclic ring having 1-2 heteroatoms independently selected from nitrogen, oxygen, and sulfur.

In some embodiments, R is an optionally substituted 4-membered saturated or partially unsaturated heterocyclic ring having 1-2 heteroatoms independently selected from nitrogen, oxygen, and sulfur. In some embodiments, R is an optionally substituted 4-membered partially unsaturated heterocyclic ring having 2 heteroatoms independently selected from nitrogen, oxygen, and sulfur. In some embodiments, R is an optionally substituted 4-membered partially unsaturated heterocyclic ring having no more than 1 heteroatom. In some embodiments, R is an optionally substituted 4-membered partially unsaturated heterocyclic ring having no more than 1 heteroatom, wherein the heteroatom is nitrogen. In some embodiments, R is an optionally substituted 4-membered partially unsaturated heterocyclic ring having no more than 1 heteroatom, wherein the heteroatom is oxygen. In some embodiments, R is an optionally substituted 4-membered partially unsaturated heterocyclic ring having no more than 1 heteroatom, wherein the heteroatom is sulfur. In some embodiments, R is an optionally substituted 4-membered partially unsaturated heterocyclic ring having 2 oxygen atoms. In some embodiments, R is an optionally substituted 4-membered partially unsaturated heterocyclic ring having 2 nitrogen atoms. In some embodiments, R is an optionally substituted 4-membered saturated or partially unsaturated heterocyclic ring having 1-2 heteroatoms independently selected from nitrogen, oxygen, and sulfur. In some embodiments, R is an optionally substituted 4-membered partially unsaturated heterocyclic ring having 2 heteroatoms independently selected from nitrogen, oxygen, and sulfur. In some embodiments, R is an optionally substituted 4-membered partially unsaturated heterocyclic ring having no more than 1 heteroatom. In some embodiments, R is an optionally substituted 4-membered partially unsaturated heterocyclic ring having no more than 1 heteroatom, wherein the heteroatom is nitrogen. In some embodiments, R is an optionally substituted 4-membered partially unsaturated heterocyclic ring having no more than 1 heteroatom, wherein the heteroatom is oxygen. In some embodiments, R is an optionally substituted 4-membered partially unsaturated heterocyclic ring having no more than 1 heteroatom, wherein the heteroatom is sulfur. In some embodiments, R is an optionally substituted 4-membered partially unsaturated heterocyclic ring having 2 oxygen atoms. In some embodiments, R is an optionally substituted 4-membered partially unsaturated heterocyclic ring having 2 nitrogen atoms.

In some embodiments, R is an optionally substituted 5-membered saturated or partially unsaturated heterocyclic ring having 1-2 heteroatoms independently selected from nitrogen, oxygen, and sulfur. In some embodiments, R is an optionally substituted 5-membered partially unsaturated heterocyclic ring having 2 heteroatoms independently selected from nitrogen, oxygen, and sulfur. In some embodiments, R is an optionally substituted 5-membered partially unsaturated heterocyclic ring having no more than 1 heteroatom. In some embodiments, R is an optionally substituted 5-membered partially unsaturated heterocyclic ring having no more than 1 heteroatom, wherein the heteroatom is nitrogen. In some embodiments, R is an optionally substituted 5-membered partially unsaturated heterocyclic ring having no more than 1 heteroatom, wherein the heteroatom is oxygen. In some embodiments, R is an optionally substituted 5-membered partially unsaturated heterocyclic ring having no more than 1 heteroatom, wherein the heteroatom is sulfur. In some embodiments, R is an optionally substituted 5-membered partially unsaturated heterocyclic ring having 2 oxygen atoms. In some embodiments, R is an optionally substituted 5-membered partially unsaturated heterocyclic ring having 2 nitrogen atoms.

In some embodiments, R is an optionally substituted 6-membered saturated or partially unsaturated heterocyclic ring having 1-2 heteroatoms independently selected from nitrogen, oxygen, and sulfur. In some embodiments, R is an optionally substituted 6-membered partially unsaturated heterocyclic ring having 2 heteroatoms independently selected from nitrogen, oxygen, and sulfur. In some embodiments, R is an optionally substituted 6-membered partially unsaturated heterocyclic ring having no more than 1 heteroatom. In some embodiments, R is an optionally substituted 6-membered partially unsaturated heterocyclic ring having no more than 1 heteroatom, wherein the heteroatom is nitrogen. In some embodiments, R is an optionally substituted 6-membered partially unsaturated heterocyclic ring having no more than 1 heteroatom, wherein the heteroatom is oxygen. In some embodiments, R is an optionally substituted 6-membered partially unsaturated heterocyclic ring having no more than 1 heteroatom, wherein the heteroatom is sulfur. In some embodiments, R is an optionally substituted 6-membered partially unsaturated heterocyclic ring having 2 oxygen atoms. In some embodiments, R is an optionally substituted 6-membered partially unsaturated heterocyclic ring having 2 nitrogen atoms.

In certain embodiments, R is a 3-7 membered saturated or partially unsaturated heterocyclic ring having 1-2 heteroatoms independently selected from nitrogen, oxygen, and sulfur. In certain embodiments, R is optionally substituted oxiranyl, oxetanyl, tetrahydrofuranyl, tetrahydropyranyl, oxepaneyl, aziridineyl, azetidineyl, pyrrolidinyl, piperidinyl, azepanyl, thiiranyl, thietanyl, tetrahydrothiophenyl, tetrahydrothiopyranyl, thiepanyl, dioxolanyl, oxathiolanyl, oxazolidinyl, imidazolidinyl, thiazolidinyl, dithiolanyl, dioxanyl, morpholinyl, oxathianyl, piperazinyl, thiomorpholinyl, dithianyl, dioxepanyl, oxazepanyl, oxathiepanyl, dithiepanyl, diazepanyl, dihydrofuranonyl, tetrahydropyranonyl, oxepanonyl, pyrolidinonyl, piperidinonyl, azepanonyl, dihydrothiophenonyl, tetrahydrothiopyranonyl, thiepanonyl, oxazolidinonyl, oxazinanonyl, oxazepanonyl, dioxolanonyl, dioxanonyl, dioxepanonyl, oxathiolinonyl, oxathianonyl, oxathiepanonyl, thiazolidinonyl, thiazinanonyl, thiazepanonyl, imidazolidinonyl, tetrahydropyrimidinonyl, diazepanonyl, imidazolidinedionyl, oxazolidinedionyl, thiazolidinedionyl, dioxolanedionyl, oxathiolanedionyl, piperazinedionyl, morpholinedionyl, thiomorpholinedionyl, tetrahydropyranyl, tetrahydrofuranyl, morpholinyl, thiomorpholinyl, piperidinyl, piperazinyl, pyrrolidinyl, tetrahydrothiophenyl, or tetrahydrothiopyranyl.

In certain embodiments, R is an optionally substituted 5-6 membered partially unsaturated monocyclic ring having 1-2 heteroatoms independently selected from nitrogen, oxygen, and sulfur. In certain embodiments, R is an optionally substituted tetrahydropyridinyl, dihydrothiazolyl, dihydrooxazolyl, or oxazolinyl group.

In some embodiments, R is an optionally substituted 7-10 membered bicyclic saturated or partially unsaturated heterocyclic ring having 1-5 heteroatoms independently selected from nitrogen, oxygen, and sulfur. In some embodiments, R is optionally substituted indolinyl. In some embodiments, R is optionally substituted isoindolinyl. In some embodiments, R is optionally substituted 1, 2, 3, 4-tetrahydroquinolinyl. In some embodiments, R is optionally substituted 1, 2, 3, 4-tetrahydroisoquinolinyl. In some embodiments, R is an optionally substituted azabicyclo[3.2.1]octanyl.

In some embodiments, R is an optionally substituted 8-10 membered bicyclic heteroaryl ring having 1-5 heteroatoms independently selected from nitrogen, oxygen, and sulfur.

In some embodiments, R is an optionally substituted 5,6-fused heteroaryl ring having 1-5 heteroatoms independently selected from nitrogen, oxygen, and sulfur. In some embodiments, R is an optionally substituted 5,6-fused heteroaryl ring having 1-4 heteroatoms independently selected from nitrogen, oxygen, and sulfur. In some embodiments, R is an optionally substituted 5,6-fused heteroaryl ring having 1-3 heteroatoms independently selected from nitrogen, oxygen, and sulfur. In some embodiments, R is an optionally substituted 5,6-fused heteroaryl ring having two heteroatoms independently selected from nitrogen, oxygen, and sulfur. In some embodiments, R is optionally substituted 1,4-dihydropyrrolo[3,2-b]pyrrolyl, 4H-furo[3,2-b]pyrrolyl, 4H-thieno[3,2-b]pyrrolyl, furo[3,2-b]furanyl, thieno[3,2-b]furanyl, thieno[3,2-b]thienyl, 1H-pyrrolo[1,2-a]imidazolyl, pyrrolo[2,1-b]oxazolyl or pyrrolo[2,1-b]thiazolyl. In some embodiments, R is an optionally substituted 5,6-fused heteroaryl ring having three heteroatoms independently selected from nitrogen, oxygen, and sulfur. In some embodiments, R is optionally substituted dihydropyrroloimidazolyl, 1H-furoimidazolyl, 1H-thienoimidazolyl, furooxazolyl, furoisoxazolyl, 4H-pyrrolooxazolyl, 4H-pyrroloisoxazolyl, thienooxazolyl, thienoisoxazolyl, 4H-pyrrolothiazolyl, furothiazolyl, thienothiazolyl, 1H-imidazoimidazolyl, imidazooxazolyl or imidazo[5,1-b]thiazolyl. In some embodiments, R is an optionally substituted 5,6-fused heteroaryl ring having four heteroatoms independently selected from nitrogen, oxygen, and sulfur. In some embodiments, R is an optionally substituted 5,6-fused heteroaryl ring having five heteroatoms independently selected from nitrogen, oxygen, and sulfur.

In some embodiments, R is an optionally substituted 5,6-fused heteroaryl ring having 1-5 heteroatoms independently selected from nitrogen, oxygen, and sulfur. In other embodiments, R is an optionally substituted 5,6-fused heteroaryl ring having 1-2 heteroatoms independently selected from nitrogen, oxygen, and sulfur. In certain embodiments, R is an optionally substituted 5,6-fused heteroaryl ring having one heteroatom independently selected from nitrogen, oxygen, and sulfur. In some embodiments, R is optionally substituted indolyl. In some embodiments, R is optionally substituted benzofuranyl. In some embodiments, R is optionally substituted benzo[b]thienyl. In certain embodiments, R is an optionally substituted 5,6-fused heteroaryl ring having two heteroatoms independently selected from nitrogen, oxygen, and sulfur. In some embodiments, R is optionally substituted azaindolyl. In some embodiments, R is optionally substituted benzimidazolyl. In some embodiments, R is optionally substituted benzothiazolyl. In some embodiments, R is optionally substituted benzoxazolyl. In some embodiments, R is an optionally substituted indazolyl. In certain embodiments, R is an optionally substituted 5,6-fused heteroaryl ring having three heteroatoms independently selected from nitrogen, oxygen, and sulfur. In some embodiments, R is optionally substituted oxazolopyridiyl, thiazolopyridinyl or imidazopyridinyl. In certain embodiments, R is an optionally substituted 5,6-fused heteroaryl ring having four heteroatoms independently selected from nitrogen, oxygen, and sulfur. In some embodiments, R is optionally substituted purinyl, oxazolopyrimidinyl, thiazolopyrimidinyl, oxazolopyrazinyl, thiazolopyrazinyl, imidazopyrazinyl, oxazolopyridazinyl, thiazolopyridazinyl or imidazopyridazinyl. In certain embodiments, R is an optionally substituted 5,6-fused heteroaryl ring having five heteroatoms independently selected from nitrogen, oxygen, and sulfur.

In certain embodiments, R is an optionally substituted 6,6-fused heteroaryl ring having 1-5 heteroatoms independently selected from nitrogen, oxygen, and sulfur. In some embodiments, R is an optionally substituted 6,6-fused heteroaryl ring having 1-2 heteroatoms independently selected from nitrogen, oxygen, and sulfur. In other embodiments, R is an optionally substituted 6,6-fused heteroaryl ring having one heteroatom selected from nitrogen, oxygen, and sulfur. In some embodiments, R is optionally substituted quinolinyl. In some embodiments, R is optionally substituted isoquinolinyl. In some embodiments, R is an optionally substituted 6,6-fused heteroaryl ring having two heteroatoms independently selected from nitrogen, oxygen, and sulfur. In some embodiments, R is optionally substituted quinazolinyl, phthalazinyl, quinoxalinyl or naphthyridinyl. In some embodiments, R is an optionally substituted 6,6-fused heteroaryl ring having three heteroatoms independently selected from nitrogen, oxygen, and sulfur. In some embodiments, R is optionally substituted pyridopyrimidinyl, pyridopyridazinyl, pyridopyrazinyl, or benzotriazinyl. In some embodiments, R is an optionally substituted 6,6-fused heteroaryl ring having four heteroatoms independently selected from nitrogen, oxygen, and sulfur. In some embodiments, R is optionally substituted pyridotriazinyl, pteridinyl, pyrazinopyrazinyl, pyrazinopyridazinyl, pyridazinopyridazinyl, pyrimidopyridazinyl or pyrimidopyrimidinyl. In some embodiments, R is an optionally substituted 6,6-fused heteroaryl ring having five heteroatoms independently selected from nitrogen, oxygen, and sulfur.

In some embodiments, R is optionally substituted C₆₋₃₀ arylaliphatic. In some embodiments, R is optionally substituted C₆₋₂₀ arylaliphatic. In some embodiments, R is optionally substituted C₆₋₁₀ arylaliphatic. In some embodiments, an aryl moiety of the arylaliphatic has 6, 10, or 14 aryl carbon atoms. In some embodiments, an aryl moiety of the arylaliphatic has 6 aryl carbon atoms. In some embodiments, an aryl moiety of the arylaliphatic has 10 aryl carbon atoms. In some embodiments, an aryl moiety of the arylaliphatic has 14 aryl carbon atoms. In some embodiments, an aryl moiety is optionally substituted phenyl.

In some embodiments, R is optionally substituted C₆₋₃₀ arylheteroaliphatic having 1-10 heteroatoms independently selected from oxygen, nitrogen, sulfur, phosphorus and silicon. In some embodiments, R is optionally substituted C₆₋₃₀ arylheteroaliphatic having 1-10 heteroatoms independently selected from oxygen, nitrogen, and sulfur. In some embodiments, R is optionally substituted C₆₋₂₀ arylheteroaliphatic having 1-10 heteroatoms independently selected from oxygen, nitrogen, sulfur, phosphorus and silicon. In some embodiments, R is optionally substituted C₆₋₂₀ arylheteroaliphatic having 1-10 heteroatoms independently selected from oxygen, nitrogen, and sulfur. In some embodiments, R is optionally substituted C₆₋₁₀ arylheteroaliphatic having 1-5 heteroatoms independently selected from oxygen, nitrogen, sulfur, phosphorus and silicon. In some embodiments, R is optionally substituted C₆₋₁₀ arylheteroaliphatic having 1-5 heteroatoms independently selected from oxygen, nitrogen, and sulfur.

In some embodiments, two R groups are optionally and independently taken together to form a covalent bond. In some embodiments, —C═O is formed. In some embodiments, —C═C— is formed. In some embodiments, —C≡C— is formed.

In some embodiments, two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon. In some embodiments, two or more R groups on the same atom are optionally and independently taken together with the atom to form an optionally substituted, 3-20 membered monocyclic, bicyclic or polycyclic ring having, in addition to the atom, 0-10 heteroatoms independently selected from oxygen, nitrogen, sulfur, phosphorus and silicon. In some embodiments, two or more R groups on the same atom are optionally and independently taken together with the atom to form an optionally substituted, 3-10 membered monocyclic, bicyclic or polycyclic ring having, in addition to the atom, 0-5 heteroatoms independently selected from oxygen, nitrogen, sulfur, phosphorus and silicon. In some embodiments, two or more R groups on the same atom are optionally and independently taken together with the atom to form an optionally substituted, 3-6 membered monocyclic, bicyclic or polycyclic ring having, in addition to the atom, 0-3 heteroatoms independently selected from oxygen, nitrogen, sulfur, phosphorus and silicon. In some embodiments, two or more R groups on the same atom are optionally and independently taken together with the atom to form an optionally substituted, 3-5 membered monocyclic, bicyclic or polycyclic ring having, in addition to the atom, 0-3 heteroatoms independently selected from oxygen, nitrogen, sulfur, phosphorus and silicon.

In some embodiments, two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon. In some embodiments, two or more R groups on two or more atoms are optionally and independently taken together with their intervening atoms to form an optionally substituted, 3-20 membered monocyclic, bicyclic or polycyclic ring having, in addition to the intervening atoms, 0-10 heteroatoms independently selected from oxygen, nitrogen, sulfur, phosphorus and silicon. In some embodiments, two or more R groups on two or more atoms are optionally and independently taken together with their intervening atoms to form an optionally substituted, 3-10 membered monocyclic, bicyclic or polycyclic ring having, in addition to the intervening atoms, 0-10 heteroatoms independently selected from oxygen, nitrogen, sulfur, phosphorus and silicon. In some embodiments, two or more R groups on two or more atoms are optionally and independently taken together with their intervening atoms to form an optionally substituted, 3-10 membered monocyclic, bicyclic or polycyclic ring having, in addition to the intervening atoms, 0-5 heteroatoms independently selected from oxygen, nitrogen, sulfur, phosphorus and silicon. In some embodiments, two or more R groups on two or more atoms are optionally and independently taken together with their intervening atoms to form an optionally substituted, 3-6 membered monocyclic, bicyclic or polycyclic ring having, in addition to the intervening atoms, 0-3 heteroatoms independently selected from oxygen, nitrogen, sulfur, phosphorus and silicon. In some embodiments, two or more R groups on two or more atoms are optionally and independently taken together with their intervening atoms to form an optionally substituted, 3-5 membered monocyclic, bicyclic or polycyclic ring having, in addition to the intervening atoms, 0-3 heteroatoms independently selected from oxygen, nitrogen, sulfur, phosphorus and silicon.

In some embodiments, heteroatoms in R groups, or in the structures formed by two or more R groups taken together, are selected from oxygen, nitrogen, and sulfur. In some embodiments, a formed ring is 3, 4, 5, 6, 7, 8, 9, 10, 11, 12, 13, 14, 15, 16, 17, 18, 19, or 20-membered. In some embodiments, a formed ring is saturated. In some embodiments, a formed ring is partially saturated. In some embodiments, a formed ring is aromatic. In some embodiments, a formed ring comprises a saturated, partially saturated, or aromatic ring moiety. In some embodiments, a formed ring comprises 5, 6, 7, 8, 9, 10, 11, 12, 13, 14, 15, 16, 17, 18, 19, or 20 aromatic ring atoms. In some embodiments, a formed contains no more than 5, 6, 7, 8, 9, 10, 11, 12, 13, 14, 15, 16, 17, 18, 19, or 20 aromatic ring atoms. In some embodiments, aromatic ring atoms are selected from carbon, nitrogen, oxygen and sulfur.

In some embodiments, a ring formed by two or more R groups (or two or more groups selected from R and variables that can be R) taken together is a C₃₋₃₀ cycloaliphatic, C₆₋₃₀ aryl, 5-30 membered heteroaryl having 1-10 heteroatoms independently selected from oxygen, nitrogen, sulfur, phosphorus and silicon, or 3-30 membered heterocyclyl having 1-10 heteroatoms independently selected from oxygen, nitrogen, sulfur, phosphorus and silicon, ring as described for R, but bivalent or multivalent.

In some embodiments, P^{L} is P(═W). In some embodiments, P^{L} IS P. In some embodiments, P^{L} is P→B(R′)₃. In some embodiments, p of P^{L} is chiral. In some embodiments, p of P^{L} is Rp. In some embodiments, p of P^{L} IS Sp. In some embodiments, a linkage of formula I is a phosphate linkage or a salt form thereof. In some embodiments, a linkage of formula I is a phosphorothioate linkage or a salt form thereof. In some embodiments, P^{L} is P*(═W), wherein P* is a chiral linkage phosphorus. In some embodiments, P^{L} is P*(═O), wherein P* is a chiral linkage phosphorus.

In some embodiments, W is O. In some embodiments, W is S. In some embodiments, W is Se.

In some embodiments, X is —O—. In some embodiments, X is —S—. In some embodiments, Y is —O—. In some embodiments, Z is —O—. In some embodiments, W is —O—, Y is —O—, Z is —O—, and X is —O— or —S—. In some embodiments, W is —S—, Y is —O—, Z is —O—, and X is —O—.

In some embodiments, R′ is R as described in the present disclosure. In some embodiments, R′ is —H. In some embodiments, R′ is not —H.

In some embodiments, —X-L-R¹ comprises or is an optionally substituted moiety of a chiral auxiliary/reagent {e.g., H—X-L-R¹ is an optionally substituted [e.g., capped (e.g., capped at a nitrogen using —C(O)R′)] chiral auxiliary/reagent}, e.g., as used in chirally controlled oligonucleotide synthesis, such as those described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, or WO 2018/098264, chiral auxiliaries/reagents of each of which are independently incorporated herein by reference. In some embodiments, H—X-L-R′ is

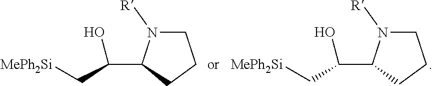

In some embodiments, H—X-L-R¹ is

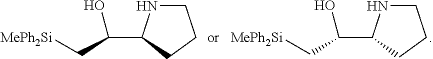

In some embodiments, H—X-L-R¹ is

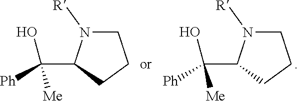

In some embodiments, H—X-L-R¹ is

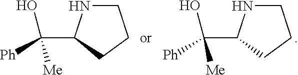 [SMILES: C[C@@](O)(c1ccccc1)[C@@H]1CCCN1 ; C[C@](O)(c1ccccc1)[C@H]1CCCN1]

In some embodiments, H—X-L-R¹ is

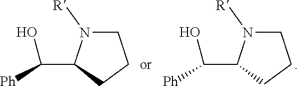

In some embodiments, H—X-L-R¹ is

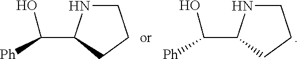 [SMILES: O[C@H](c1ccccc1)[C@@H]1CCCN1 ; O[C@@H](c1ccccc1)[C@H]1CCCN1]

In some embodiments, R′ is —C(O)R. In some embodiments, R′ is —C(O)CH₃.

In some embodiments, a provided oligonucleotide composition, e.g., a chirally controlled oligonucleotide composition, an HTT oligonucleotide composition, etc., comprises a plurality of oligonucleotides each of which is an oligonucleotide of formula O-I or a salt thereof. In some embodiments, an oligonucleotide of formula O-I comprise chemical modifications (e.g., sugar modifications, base modifications, modified internucleotidic linkages, etc., and patterns thereof), stereochemistry (e.g., of chiral linkage phosphorus, etc., and patterns thereof), base sequences, etc., as described in the present disclosure. In some embodiments, a chirally controlled oligonucleotide composition of oligonucleotides of formula O-I is a chirally controlled oligonucleotide composition of an oligonucleotide selected from in Table 1, etc., wherein the oligonucleotide comprises at least one chirally controlled internucleotidic linkage.

In some embodiments, z is 1-1000. In some embodiments, z+1 is an oligonucleotide length as described in the present disclosure. In some embodiments, z is no less than 4, 5, 6, 7, 8, 9, 10, 11, 12, 13, 14, 15, 16, 17, 18, or 19. In some embodiments, z is no less than 4, 5, 6, 7, 8, 9, 10, 11, 12, 13, or 14. In some embodiments, z is no more than 50, 60, 70, 80, 90, 100, 150, or 200. In some embodiments, z is 5-50, 10-50, 14-50, 14-45, 14-40, 14-35, 14-30, 14-25, 14-100, 14-150, 14-200, 14-250, 14-300, 15-50, 15-45, 15-40, 15-35, 15-30, 15-25, 15-100, 15-150, 15-200, 15-250, 15-300, 16-50, 16-45, 16-40, 16-35, 16-30, 16-25, 16-100, 16-150, 16-200, 16-250, 16-300, 17-50, 17-45, 17-40, 17-35, 17-30, 17-25, 17-100, 17-150, 17-200, 17-250, 17-300, 18-50, 18-45, 18-40, 18-35, 18-30, 18-25, 18-100, 18-150, 18-200, 18-250, 18-300, 19-50, 19-45, 19-40, 19-35, 19-30, 19-25, 19-100, 19-150, 19-200, 19-250, or 19-300. In some embodiments, z is 10. In some embodiments, z is 11. In some embodiments, z is 12. In some embodiments, z is 13. In some embodiments, z is 14. In some embodiments, z is 15. In some embodiments, z is 16. In some embodiments, z is 17. In some embodiments, z is 18. In some embodiments, z is 19. In some embodiments, z is 20. In some embodiments, z is 21. In some embodiments, z is 22. In some embodiments, z is 23. In some embodiments, z is 24. In some embodiments, z is 25. In some embodiments, z is 26. In some embodiments, z is 27. In some embodiments, z is 28. In some embodiments, z is 29. In some embodiments, z is 30. In some embodiments, z is 31. In some embodiments, z is 32. In some embodiments, z is 33. In some embodiments, z is 34.

In some embodiments, Ring A^{L} is bivalent. In some embodiments, Ring A^{L} is polyvalent. In some embodiments, Ring A^{L} is bivalent and is -Cy-. In some embodiments, Ring A^{L} is an optionally substituted bivalent triazole ring. In some embodiments, Ring A^{L} is trivalent and is Cy^{L}. In some embodiments, Ring A^{L} is tetravalent and is Cy^{L}. In some embodiments, Ring A^{L} is optionally substituted

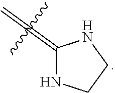 [SMILES: *=C1NCCN1]

In some embodiments, —X-L-R¹ is optionally substituted alkynyl. In some embodiments, —X-L-R¹ is —C≡C—. In some embodiments, an alkynyl group, e.g., —C≡C—, can react with a number of reagents through various reactions to provide further modifications. For example, in some embodiments, an alkynyl group can react with azides through click chemistry. In some embodiments, an azide has the structure of R¹—N₃.

In some embodiments, g is 0-20. In some embodiments, g is 1-20. In some embodiments, g is 1-5. In some embodiments, g is 1. In some embodiments, g is 2. In some embodiments, g is 3. In some embodiments, g is 4. In some embodiments, g is 5. In some embodiments, g is 6. In some embodiments, g is 7. In some embodiments, g is 8. In some embodiments, g is 9. In some embodiments, g is 10. In some embodiments, g is 11. In some embodiments, g is 12. In some embodiments, g is 13. In some embodiments, g is 14. In some embodiments, g is 15. In some embodiments, g is 16. In some embodiments, g is 17. In some embodiments, g is 18. In some embodiments, g is 19. In some embodiments, g is 20.

In some embodiments,

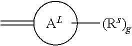

is

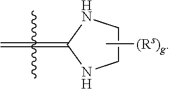

In some embodiments,

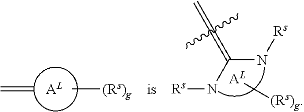

In some embodiments,

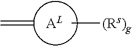

is

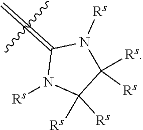

In some embodiments, t is 1, 2, 3, 4, 5, 6, 7, 8, 9, 10, 11, 12, 13, 14, 15, 16, 17, 18, 19, 20, 21, 22, 23, 24, or 25. In some embodiments, t is 1, 2, 3, 4, 5, 6, 7, 8, 9, or 10. In some embodiments, t is 1-10, 1-15, 1-20, 1-25, 1-30, 2-10, 2-15, 2-20, 2-25, 2-30, 5-10, 5-15, 5-20, 5-25, 5-30, 8-10, 8-15, 8-20, 8-25, 8-30, 10-15, 10-20, 10-25, or 10-30. In some embodiments, t is 1-3, 1-4, 1-5, 1-10, 2-3, 2-5, 2-6, or 2-10. In some embodiments, t is 1. In some embodiments, t is 2. In some embodiments, t is 3. In some embodiments, t is 4. In some embodiments, t is 5. In some embodiments, t is 6. In some embodiments, t is 7. In some embodiments, t is 8. In some embodiments, t is 9. In some embodiments, t is 10. In some embodiments, t is 11. In some embodiments, t is 12. In some embodiments, t is 13. In some embodiments, t is 14. In some embodiments, t is 15. In some embodiments, t is 16. In some embodiments, t is 17. In some embodiments, t is 18. In some embodiments, t is 19. In some embodiments, t is 20.

In some embodiments, m is 1, 2, 3, 4, 5, 6, 7, 8, 9, 10, 11, 12, 13, 14, 15, 16, 17, 18, 19, 20, 21, 22, 23, 24, or 25. In some embodiments, m is 1, 2, 3, 4, 5, 6, 7, 8, 9, or 10. In some embodiments, m is 1-10, 1-15, 1-20, 1-25, 1-30, 2-10, 2-15, 2-20, 2-25, 2-30, 5-10, 5-15, 5-20, 5-25, 5-30, 8-10, 8-15, 8-20, 8-25, 8-30, 10-15, 10-20, 10-25, or 10-30. In some embodiments, m is 1-3, 1-4, 1-5, 1-10, 2-3, 2-5, 2-6, or 2-10. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16. In some embodiments, m is 17. In some embodiments, m is 18. In some embodiments, m is 19. In some embodiments, m is 20.

In some embodiments, t=m. In some embodiments, t>m. In some embodiments, t<m. In some embodiments, n is 1-10, 1-15, 1-20, 1-25, 1-30, 2-10, 2-15, 2-20, 2-25, 2-30, 5-10, 5-15, 5-20, 5-25, 5-30, 8-10, 8-15, 8-20, 8-25, 8-30, 10-15, 10-20, 10-25, or 10-30. In some embodiments, n is 1, 2, 3, 4, 5, 6, 7, 8, 9, 10, 11, 12, 13, 14, 15, 16, 17, 18, 19, 20, 21, 22, 23, 24, or 25. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, n is 11. In some embodiments, n is 12. In some embodiments, n is 13. In some embodiments, n is 14. In some embodiments, n is 15. In some embodiments, n is 16. In some embodiments, n is 17. In some embodiments, n is 18. In some embodiments, n is 19. In some embodiments, n is 20.

In some embodiments, x is 1, 2, 3, 4, 5, 6, 7, 8, 9, 10, 11, 12, 13, 14, 15, 16, 17, 18, 19, 20, 21, 22, 23, 24, or 25. In some embodiments, x is 1, 2, 3, 4, 5, 6, 7, 8, 9, or 10. In some embodiments, x is 1-10, 1-15, 1-20, 1-25, 1-30, 2-10, 2-15, 2-20, 2-25, 2-30, 5-10, 5-15, 5-20, 5-25, 5-30, 8-10, 8-15, 8-20, 8-25, 8-30, 10-15, 10-20, 10-25, or 10-30. In some embodiments, x is 1-3, 1-4, 1-5, 1-10, 2-3, 2-5, 2-6, or 2-10. In some embodiments, x is 1. In some embodiments, x is 2. In some embodiments, x is 3. In some embodiments, x is 4. In some embodiments, x is 5. In some embodiments, x is 6. In some embodiments, x is 7. In some embodiments, x is 8. In some embodiments, x is 9. In some embodiments, x is 10. In some embodiments, x is 11. In some embodiments, x is 12. In some embodiments, x is 13. In some embodiments, x is 14. In some embodiments, x is 15. In some embodiments, x is 16. In some embodiments, x is 17. In some embodiments, x is 18. In some embodiments, x is 19. In some embodiments, x is 20.

In some embodiments, y is 1, 2, 3, 4, 5, 6, 7, 8, 9, 10, 11, 12, 13, 14, 15, 16, 17, 18, 19, 20, 21, 22, 23, 24, or 25. In some embodiments, y is 1, 2, 3, 4, 5, 6, 7, 8, 9, or 10. In some embodiments, y is 1-10, 1-15, 1-20, 1-25, 1-30, 2-10, 2-15, 2-20, 2-25, 2-30, 5-10, 5-15, 5-20, 5-25, 5-30, 8-10, 8-15, 8-20, 8-25, 8-30, 10-15, 10-20, 10-25, or 10-30. In some embodiments, y is 1-3, 1-4, 1-5, 1-10, 2-3, 2-5, 2-6, or 2-10. In some embodiments, y is 1. In some embodiments, y is 2. In some embodiments, y is 3. In some embodiments, y is 4. In some embodiments, y is 5. In some embodiments, y is 6. In some embodiments, y is 7. In some embodiments, y is 8. In some embodiments, y is 9. In some embodiments, y is 10. In some embodiments, y is 11. In some embodiments, y is 12. In some embodiments, y is 13. In some embodiments, y is 14. In some embodiments, y is 15. In some embodiments, y is 16. In some embodiments, y is 17. In some embodiments, y is 18. In some embodiments, y is 19. In some embodiments, y is 20.

In some embodiments, a number following an oligonucleotide designation indicates a batch. For example, in some embodiments, WV-#####-01 indicates batch 01 of oligonucleotide WV-#####.

### EXEMPLIFICATION

Certain examples of provided technologies (compounds (oligonucleotides, reagents, etc.), compositions, methods (methods of preparation, use, assessment, etc.), etc.) were presented herein.

### Example 1. Oligonucleotide Synthesis

Various technologies for preparing oligonucleotides and oligonucleotide compositions (both stereorandom and chirally controlled) are known and can be utilized in accordance with the present disclosure, including, for example, those in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, or WO 2018/237194, the methods and reagents of each of which are incorporated herein by reference.

In some embodiments, oligonucleotides are prepared using suitable chiral auxiliaries, e.g., DPSE chiral auxiliaries. One example oligonucleotide preparation is described below. Various oligonucleotides, e.g., those in Table 1, and compositions thereof, can be prepared similarly in accordance with the present disclosure. As appreciated by those skilled in the art, conditions (e.g., reagents, solvents, reaction time, etc.) may be altered to achieve desired yields and/or purities for various steps and/or overall syntheses of various oligonucleotides.

In one example oligonucleotide preparation, synthesis was performed on an ÄKTA OP100 synthesizer (GE Healthcare) using a 3.5 cm diameter stainless steel column reactor on a 873 μmol scale using CPG support (loading 75 umol/g). Those skilled in the art will appreciate that other synthesizer, column and support can also be suitable. Typically, five-step cycles were utilized (detritylation, coupling, capping 1, oxidation/thiolation and capping 2).

Detritylation was typically performed in acidic conditions, for example, using 3% DCA in toluene with a monitoring system, e.g., UV watch command set at 436 nm. Following detritylation, detritylation reagent and released product in solution were washed away. For example, in some cases, at least 4 column volumes (CV) of ACN were used to wash off the detritylation reagent.

For coupling, phosphoramidites and activators (e.g., CMIMT and ETT) were dissolved in suitable solvents, and the solutions were prepared and dried, e.g., over 3 Å molecular sieves, for a sufficient period of time (e.g., at least 4 hours) prior to synthesis. Phosphoramidites coupling were performed at suitable amidite and activator concentrations. In one example run, DPSE amidites coupling was performed using 0.2 M amidite solutions and 0.6 M CMIMT. All amidites were dissolved in suitable solvents, e.g., ACN, except that dC-L and dC-D amidites were usually dissolved in isobutyronitrile (IBN). DPSE MOE amidites were often dissolved in 20% IBN/ACN v/v. CMIMT was typically dissolved in ACN. In some cases, using a suitable amount, e.g., 2.5 equivalents, coupling was performed by mixing 33% (by volume) of the respective amidite solutions with 67% of the CMIMT activator in-line prior to addition to the column. Coupling mixtures were typically recirculated for a period of time, e.g., a minimum of 6 minutes, to maximize the coupling efficiency. In some embodiments, PSM amidites can be utilized for coupling wherein the PSM chiral auxiliaries may be optionally removed later under, e.g., a basic condition. In some embodiments, an azido imidazolinium salt (e.g., 2-azido-1,3-dimethylimidazolinium hexafluorophosphate) can be utilized for modification to prepare a neutral internucleotidic linkage (e.g., n001).

Standard CED amidite coupling was typically performed using 0.2 M amidite solutions and 0.6 M ETT in ACN. MOE-T amidite was typically dissolved in 20% IBN/ACN v/v. In some cases, using a suitable amount, e.g., 2.5 equivalents, coupling was performed by mixing 40% (by volume) of the respective amidite solution with 60% of the ETT activator in-line prior to addition to the column. Coupling mixtures were typically recirculated for a period of time, e.g., a minimum of 8 minutes, to maximize the coupling efficiency.

After coupling, the column was washed with a suitable amount of a suitable solvent, e.g., with 2 CV of ACN.

For DPSE couplings, the column was then treated with a suitable capping solution at a suitable amount for a sufficient period of time, e.g., Capping 1 solution (Capping A: Acetic Anhydride/Lutidine/ACN 10/10/80 v/v/v) mixture for 1 CV in 4 minutes to cap (e.g., acetylate) the chiral axillary amine. Following this step, the column was washed with a suitable solvent at a suitable volume, e.g., ACN for at least 2 CV. Modification, e.g., thiolation was then performed with a suitable reagent under a suitable condition, e.g., for thiolation, 0.1 M Xanthane Hydride in pyridine/ACN (1:1) with a contact time of 6 min for 1.2 CV. After thiolation the column was washed using a sufficient amount of a suitable solvent, e.g., 2 CV CAN. Capping 2 was performed using a suitable condition, e.g., 0.4 CV of Capping A and Capping B (16% n-methylimidazole in ACN) reagents mixed inline (1:1) for a suitable time (e.g., 0.8 min) followed by a wash with a sufficient amount of a suitable solvent (e.g., 2 CV ACN wash).

For standard CED coupling cycles, there was typically no Capping 1 step. Oxidation was performed under a suitable condition, e.g., using 50 mM Iodine in/Pyridine/H₂O (9:1) for 1.5 min and 3.5 equivalents. After wash, e.g., with 2 CV ACN, capping 2 was performed using a suitable condition, e.g., 0.4 CV of Capping A and Capping B reagents mixed inline (1:1) for 0.8 min followed by a wash with a sufficient amount of a suitable solvent (e.g., 2 CV ACN wash).

Multiple cycles were performed to achieve the desired oligonucleotide sequence.

Cleavage and Deprotection: Various technologies can be utilized to remove cyanoethyl (CNET) groups in stereorandom internucleotidic linkages, for example, in one preparation they were removed by on-column treatment with 20% DEA for 15 min over 5 CV. The support was then dried, typically under a steady stream of an inert gas, e.g., nitrogen, for a period of time (e.g., 15 min). After drying, the column was unpacked, and the support transferred into a suitable container, e.g., an 800 mL pressure bottle. DPSE groups were then removed under a suitable condition, e.g., by treating the oligonucleotides-bound solid support with a 1 M solution of TEA-HF made by mixing DMSO, Water, TEA and TEA-3HF in a v/v ratio of 39:8:1:2.5, to make a 100 mL solution per mmol of oligonucleotide. The mixture was then shaken at 25° C. for a period of time, e.g., 6 hours in an incubator shaker. The mixture was cooled (ice bath) then a suitable amount of a base was added, e.g., 200 mL of aqueous ammonia per mmol of oligonucleotide. The mixture was then shaken at a suitable temperature, e.g., 45° C., for a suitable period of time, e.g., 16 hours. The mixture was then filtered (0.2-1.2 μm filters) and the cake rinsed with water. The filtrate liquor was obtained and analyzed by UPLC and a purity of 45% FLP obtained—among other things, technologies of the present disclosure can deliver chirally controlled oligonucleotides with high yields and/or crude purity. Product oligonucleotides can be characterized and quantified using a number of technologies, e.g., HPLC, LCMS, HRMS, etc. Quantification may be performed utilizing a number of technologies available in the art. In one preparation, quantification was done using a NanoDrop one spectrophotometer (Thermo Scientific). As an example, in a preparation a yield of 80,000 OD was obtained.

Purification and Desalting: Many technologies can be utilized to purify and/or desalt oligonucleotides. In one procedure, crude oligonucleotide was loaded on to an Agilent Load & Lock column (2.5 cm×30 cm) packed with TSKgel 15Q (TOSOH Biosciences). Purification was performed on an ÄKTA 150 Pure (GE Healthcare) using 20 mM NaOH and 2.5 M NaCl as eluents. Fractions were analyzed and pooled to obtain material with a purity of ≥85% FLP. The purified material was then desalted on 2K re-generated cellulose membranes followed by lyophilization to obtain the oligonucleotide as a white powder. The material can be used for various purposes, including for conjugation with additional chemical moieties, e.g., addition of Mod001 and Mod083 described below.

### Example 2. Provided Oligonucleotides can Effectively Reduce Levels of their Targets

Various technologies can be utilized to assess properties and/or activities of provided oligonucleotides and compositions thereof. Some such technologies are described in this Example. Those skilled in the art appreciate that many other technologies can be readily utilized. As demonstrated herein, provided oligonucleotides and compositions, among other things, can be highly active, e.g., in reducing levels of their target HTT nucleic acids.

Various HTT oligonucleotides were designed and constructed, including a set of human/NHP (non-human primate) HTT sequences (a subset of which have 0 or 1 mismatch from the corresponding mouse HTT sequence), and a set of mouse/rat HTT sequences (a subset of which have 1 mismatch from the corresponding human/NHP sequence). A number of HTT oligonucleotides were tested, including testing knockdown of HTT in vitro in cells at one or a range of concentrations, and IC₅₀.

Cells used include human and mouse cells. In some cases, iPSC neurons were used. In some cases, Neuro2a cells or other cells were used.

Example protocol for in vitro determination of HTT oligonucleotide activity and IC₅₀ values: For determination of HTT oligonucleotide activity, different concentrations of oligonucleotides were transfected into human or mouse cells, using 96-well plates, approximately 15,000 cells/well, using Lipofectamine 2000 (Invitrogen) as recommended by the manufacturer. Following 24 or 48 h treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). mRNA knockdown levels were calculated as % mRNA remaining relative to mock treatment (ΔΔCt) and IC50 values were determined by three parameter curve fitting of oligonucleotide concentration vs. % mRNA remaining.

In some experiments, oligonucleotides were delivered using lipofectamine or delivered gymnotically (e.g., via free uptake). In various screening assays, oligonucleotides were tested at a concentration of 10 uM and delivered gymnotically. In some experiments, the residual HTT mRNA level (after delivery of oligonucleotide) was tested relative to a standard which is the level of expression of a gene other than HTT. For some experiments, results of replicates are shown.

In some experiments, tested oligonucleotides have a wing-core-wing format. In some experiments, tested oligonucleotides have a symmetric or asymmetric format (e.g., wherein the 5′ and 3′ wings have the same or different sugar modifications and patterns thereof, respectively).

Details of various HTT oligonucleotides are provided in Table 1 herein.

HTT oligonucleotides were tested in vitro in cells, at a concentration of 5 nM, 24 h duration (e.g., HTT mRNA levels were determined 24 hrs after treatment of the cells with oligonucleotides). Numbers indicate relative amount of hHTT (human HTT) mRNA remaining, relative to hSFRS9 standard. In some tables: 100.0 will represent 100% hHTT mRNA remaining (0.0% knockdown); and 0.0 will represent 0.0% hHTT mRNA remaining (100.0% knockdown).

In some experiments, various HTT oligonucleotides were tested for selectivity (mu HTT versus wt HTT) in a Dual Luciferase Assay, as detailed in WO2017015555 and WO2017192664. In brief, some of these experiments used the protocol of: Cotransfection of mu or wt vector (psiCHECK2) containing 250 nucleotide fragment including the mu or wt isoform of the SNP with HTT oligonucleotide in Cos7 cells; Exposure time: 24 or 48 hrs; Measurement of Luminescence Renilla/Firefly using dual luciferase assay (Promega, Madison, Wis.); and Normalization of R/F from HTT oligonucleotides to R/F of -ve control.

### Neuronal Activity Assay

Human iPSC-derived neurons were plated on Matrigel® (Corning, Corning, N.Y., USA) coated 384-well plates using the Agilent Bravo liquid handling platform (Agilent, Santa Clara, Calif., USA).
24 hours after plating, media was replaced with fresh media containing ASO at a fixed concentration and cells were allowed to incubate with ASO for 7 days under gymnotic (free uptake) conditions.
On day 7 after treatment, cells were lysed and mRNA was quantified using a QuantiGene™ Singleplex branched DNA assay (Thermo Fisher, Waltham, Mass., USA).
Human HTT mRNA was quantified and levels were normalized using human tubulin. Data were expressed as fold change relative to non-targeting control.

### Selective Reporter Assay

Fragments of the human HTT gene (NM_002111) containing SNPs of interest were cloned into the psiCHECK™-2 vector system (Promega, Madison, Wis., USA) in the 3′-untranslated region (UTR) of the Renilla luciferase gene (hRluc).
Vectors containing either the mutant or wild-type SNP were cotransfected into monkey kidney-derived COS-7 cells with the ASOs at concentrations ranging from 0.03-50 nM in 96-well plates.
48 hours after transfection, plates were processed with the Dual-Glo® Luciferase assay system (Promega); selectivity of ASOs was determined based on the relative levels of Renilla luciferase versus the internal control, firefly luciferase.
   In some in vitro experiments, various HTT oligonucleotides were tested in HEK293 cells.
   In some in vitro experiments, a control oligonucleotide (at times designated a cASO) which does not target HTT was used. In some in vitro experiments, a negative control oligonucleotide was WV-9491, which does not target HTT.

Some HTT oligonucleotides were also tested in mice (e.g., C57BL6 wild type mice or other mice).

In vivo determination of HTT oligonucleotide activity: All animal procedures were performed under IACUC guidelines at Biomere (Worcester, Mass.). Male 6-8 weeks of age C57BL/6 mice were dose at 10 mL/kg at desired oligonucleotide concentration on Day 1 by subcutaneous administration to the interscapular area. Animals were euthanized (e.g., on Day 8) by CO₂ asphyxiation followed by cardiac perfusion with saline, and liver samples were harvested and flash-frozen in dry ice. Total RNA extraction, cDNA production and qPCR measurements were performed as described for in vitro oligonucleotide activity determination.

### In Vivo Studies

HD mice that express a full-length human mHTT gene with expanded CAG repeats were treated with 2 intracerebroventricular (ICV) 50-μg doses of ASOs and euthanized 7 days after the last dose. HTT levels were quantified in using QuantiGene™ Singleplex branched DNA assay (Thermo Fisher) and normalized to mouse tubulin. Data were expressed as fold change relative to non-targeting control.
   Various control oligonucleotides were used (including in data not shown), including:

| Oligo- | | Stereo- | |
|---|---|---|---|
| nucleotide | Sequence | chemistry | Comment |
| WV-1061 | mG * mU * mA * mG * mG * A * G * T * A | XXXXXXXXXX | +ve Luciferase |
| | * G * T * G * A * A * A * mG * mG * mC * | XXXXXXXXX | control for |
| | mC * mA | | psiCHECK2 |
| WV-1062 | mGmUmAmGmG * A * G * T * A * G * T * | OOOOXXXXXX | +ve Luciferase |
| | G * A * A * A * mGmGmCmCmA | XXXXXOOOO | control for |
| | | | psiCHECK2 |
| WV-1063 | mG * mU * mA * mG * mG * A * G * T * A | XXXXXXXXXX | +ve Luciferase |
| | * G * T * G * A * A * A * G * G * C * C | XXXXXXXXX | control for |
| | * A | | psiCHECK2 |
| WV-1064 | mC * mU * mC * mU * mU * A * C * T * G | XXXXXXXXXX | Negative Luciferase |
| | * T * G * C * T * G * T * mG * mG* mA * | XXXXXXXXX | control for |
| | mC * mA | | psiCHECK2 |
| WV-1065 | mCmUmCmUmU * A * C * T * G * T * G * | OOOOXXXXXX | Negative Luciferase |
| | C * T * G * T * mGmGmAmCmA | XXXXXOOOO | control for |
| | | | psiCHECK2 |
| WV-1066 | mC * mU * mC * mU * mU * A * C * T * G | XXXXXXXXXX | Negative Luciferase |
| | * T * G * C * T * G * T * G * G * A * | XXXXXXXXX | control for |
| | C * A | | psiCHECK2 |

| Oligo- | | Stereo- |
|---|---|---|
| nucleotide | Sequence | chemistry |
| WV-2376 | mC * mCmUmUmC * C * C * T * G * A * A * G * G * T | XOOOXXXXXX |
| | * T * mCmCmUmC * mC | XXXXXOOOX |
| WV-2431 | mC * SmCmUmUmC * SC * SC * ST * SG * SA * SA * | SOOOSSSSSS |
| | SG * RG * ST * ST * SmCmCmUmC * SmC | SRSSSOOOS |

Various HTT oligonucleotides were tested for their ability to knockdown the activity, level and/or expression of wild-type and/or mutant HTT mRNA or protein.

Table 2. Activity of Certain Oligonucleotides.

HTT oligonucleotides comprising a SNP at Position 11 were tested in vitro for ability to knock down wild-type (wt) and the mutant (in) HTT corresponding to the SNP. The oligonucleotides differ in chemistry and stereochemistry (or patterns thereof). Oligonucleotides were tested at 30 nM, 3 nM or 0.3 nM, and numbers represent percentage of HTT (wt or m) remaining after oligonucleotide treatment, represented as percentage of Renilla/Firefly ratio compared to control. Results from replicate data are shown. Numbers indicate the % of HTT remaining (relative to control) at the indicated oligonucleotide concentrations. 1.0 would represent 100% HTT level (0% knockdown) and 0.0 would represent 0% HTT level (100% knock down).

TABLE 3
Activity of certain oligonucleotides.

| | | |
|---|---|---|
| Oligonucleotide | Mutant | Wildtype |

30 nM

| | | | | | | |
|---|---|---|---|---|---|---|
| ONT-437 | 0.98 | 1.01 | 1.00 | 1.02 | 0.96 | 1.00 |
| ONT-211 | 1.00 | 0.98 | 1.03 | 0.99 | 1.00 | 1.02 |
| WV-918 | 0.05 | 0.04 | 0.04 | 0.14 | 0.16 | 0.14 |
| WV-922 | 0.10 | 0.10 | 0.10 | 0.12 | 0.11 | 0.12 |
| WV-938 | 0.77 | 0.81 | 0.83 | 0.97 | 0.91 | 0.96 |

3 nM

| | | | | | | |
|---|---|---|---|---|---|---|
| ONT-437 | 1.09 | 1.00 | 0.97 | 0.96 | 1.00 | 1.19 |
| ONT-211 | 0.96 | 1.00 | 1.01 | 0.93 | 1.00 | 1.00 |
| WV-918 | 0.43 | 0.41 | 0.41 | 0.72 | 0.73 | 0.74 |
| WV-922 | 0.35 | 0.33 | 0.33 | 0.43 | 0.43 | 0.48 |
| WV-938 | 1.05 | 1.02 | 1.02 | 1.03 | 1.02 | 1.12 |

0.3 nM

| | | | | | | |
|---|---|---|---|---|---|---|
| ONT-437 | 1.16 | 0.90 | 1.00 | 0.91 | 1.00 | 1.09 |
| ONT-211 | 1.24 | 0.99 | 1.00 | 0.97 | 1.00 | 1.18 |
| WV-918 | 1.09 | 1.03 | 1.10 | 1.03 | 1.09 | 1.16 |
| WV-922 | 1.17 | 1.02 | 1.00 | 0.95 | 1.07 | 1.13 |
| WV-938 | 1.15 | 0.99 | 1.00 | 0.96 | 0.89 | 1.11 |

Various HTT oligonucleotides comprising a SNP at various positions (P08 to P13 counting from the 5′ end), and different patterns of stereochemistry and/or different 2′-modifications (or patterns thereof) were tested in vitro for their ability to knock down wild-type (wt) and the mutant (in) HTT corresponding to the SNP.
Results are shown below. Cells were treated with oligonucleotides at concentrations of 3.3 nM, 10 nM or 30 nM. Numbers represent % of muHTT or wtHTT mRNA left after treatment with oligonucleotides; numbers are averages of replicate experiments and are approximate. 100.0 would represent 100% HTT level (0% knockdown) and 0.0 would represent 0% HTT level (100% knock down).

TABLE 4
Activity of certain oligonucleotides.
P08

| | |
|---|---|
| 3.3 nM | 10 nM |

| | | | | | |
|---|---|---|---|---|---|
| WV-909 | 78 | muHTT | WV-909 | 69 | muHTT |
| | 80 | wtHTT | | 81 | wtHTT |
| WV-915 | 72 | muHTT | WV-915 | 42 | muHTT |
| | 86 | wtHTT | | 60 | wtHTT |
| WV-921 | 81 | muHTT | WV-921 | 62 | muHTT |
| | 84 | wtHTT | | 55 | wtHTT |
| WV-925 | 78 | muHTT | WV-925 | 55 | muHTT |
| | 76 | wtHTT | | 42 | wtHTT |
| WV-929 | 78 | muHTT | WV-929 | 59 | muHTT |
| | 86 | wtHTT | | 63 | wtHTT |
| WV-935 | 80 | muHTT | WV-935 | 72 | muHTT |
| | 86 | wtHTT | | 89 | wtHTT |
| WV-941 | 82 | muHTT | WV-941 | 84 | muHTT |
| | 89 | wtHTT | | 83 | wtHTT |

30 nM

| | | |
|---|---|---|
| WV-909 | 43 | muHTT |
| | 72 | wtHTT |
| WV-915 | 23 | muHTT |
| | 46 | wtHTT |
| WV-921 | 28 | muHTT |
| | 40 | wtHTT |
| WV-925 | 23 | muHTT |
| | 28 | wtHTT |
| WV-929 | 33 | muHTT |
| | 42 | wtHTT |
| WV-935 | 52 | muHTT |
| | 70 | wtHTT |
| WV-941 | 68 | muHTT |
| | 77 | wtHTT |

P09

| 3.3 nM | | | 10 nM | | |
|---|---|---|---|---|---|
| WV-908 | 92 | muHTT | WV-908 | 64 | muHTT |
| | 100 | wtHTT | | 89 | wtHTT |
| WV-920 | 60 | muHTT | WV-920 | 36 | muHTT |
| | 119 | wtHTT | | 72 | wtHTT |
| WV-924 | 66 | muHTT | WV-924 | 34 | muHTT |
| | 97 | wtHTT | | 45 | wtHTT |
| WV-928 | 104 | muHTT | WV-928 | 51 | muHTT |
| | 102 | wtHTT | | 74 | wtHTT |
| WV-934 | 116 | muHTT | WV-934 | 52 | muHTT |
| | 115 | wtHTT | | 89 | wtHTT |
| WV-940 | 111 | muHTT | WV-940 | 88 | muHTT |
| | 120+ | wtHTT | | 103 | wtHTT |

30 nM

| | | |
|---|---|---|
| WV-908 | 24 | muHTT |
| | 67 | wtHTT |
| WV-920 | 15 | muHTT |
| | 35 | wtHTT |
| WV-924 | 17 | muHTT |
| | 23 | wtHTT |
| WV-928 | 18 | muHTT |
| | 40 | wtHTT |
| WV-934 | 22 | muHTT |
| | 52 | wtHTT |
| WV-940 | 49 | muHTT |
| | 66 | wtHTT |

P10

| | |
|---|---|
| 3.3 nM | 10 nM |

| | | | | | |
|---|---|---|---|---|---|
| WV-907 | 70 | muHTT | WV-907 | 42 | muHTT |
| | 112 | wtHTT | | 70 | wtHTT |
| WV-913 | 69 | muHTT | WV-913 | 29 | muHTT |
| | 97 | wtHTT | | 45 | wtHTT |
| WV-919 | 54 | muHTT | WV-919 | 20 | muHTT |
| | 80 | wtHTT | | 40 | wtHTT |
| WV-923 | 89 | muHTT | WV-923 | 64 | muHTT |
| | 85 | wtHTT | | 60 | wtHTT |
| WV-927 | 70 | muHTT | WV-927 | 38 | muHTT |
| | 92 | wtHTT | | 50 | wtHTT |
| WV-933 | 70 | muHTT | WV-933 | 41 | muHTT |
| | 117 | wtHTT | | 62 | wtHTT |
| WV-939 | 113 | muHTT | WV-939 | 74 | muHTT |
| | 99 | wtHTT | | 92 | wtHTT |

30 nM

| | | |
|---|---|---|
| WV-907 | 16 | muHTT |
| | 62 | wtHTT |
| WV-913 | 8 | muHTT |
| | 25 | wtHTT |
| WV-919 | 9 | muHTT |
| | 15 | wtHTT |
| WV-923 | 29 | muHTT |
| | 37 | wtHTT |
| WV-927 | 24 | muHTT |
| | 27 | wtHTT |
| WV-933 | 17 | muHTT |
| | 37 | wtHTT |
| WV-939 | 60 | muHTT |
| | 94 | wtHTT |

P11

| | |
|---|---|
| 3.3 nM | 10 nM |

| | | | | | |
|---|---|---|---|---|---|
| WV-906 | 94 | muHTT | WV-906 | 60 | muHTT |
| | 99 | wtHTT | | 98 | wtHTT |
| WV-918 | 68 | muHTT | WV-918 | 37 | muHTT |
| | 99 | wtHTT | | 71 | wtHTT |
| WV-922 | 70 | muHTT | WV-922 | 48 | muHTT |
| | 72 | wtHTT | | 57 | wtHTT |
| WV-926 | 89 | muHTT | WV-926 | 60 | muHTT |
| | 88 | wtHTT | | 85 | wtHTT |
| WV-932 | 78 | muHTT | WV-932 | 47 | muHTT |
| | 80 | wtHTT | | 72 | wtHTT |
| WV-938 | 92 | muHTT | WV-938 | 91 | muHTT |
| | 96 | wtHTT | | 94 | wtHTT |

30 nM

| | | |
|---|---|---|
| WV-906 | 35 | muHTT |
| | 64 | wtHTT |
| WV-918 | 15 | muHTT |
| | 31 | wtHTT |
| WV-922 | 21 | muHTT |
| | 29 | wtHTT |
| WV-926 | 23 | muHTT |
| | 47 | wtHTT |
| WV-932 | 21 | muHTT |
| | 44 | wtHTT |
| WV-938 | 49 | muHTT |
| | 90 | wtHTT |

P12

| | |
|---|---|
| 3.3 nM | 10 nM |

| | | | | | |
|---|---|---|---|---|---|
| WV-905 | 90 | muHTT | WV-905 | 72 | muHTT |
| | 92 | wtHTT | | 77 | wtHTT |
| WV-911 | 91 | muHTT | WV-911 | 46 | muHTT |
| | 97 | wtHTT | | 59 | wtHTT |
| WV-917 | 87 | muHTT | WV-917 | 51 | muHTT |
| | 102 | wtHTT | | 71 | wtHTT |
| WV-931 | 93 | muHTT | WV-931 | 69 | muHTT |
| | 94 | wtHTT | | 101 | wtHTT |
| WV-937 | 88 | muHTT | WV-937 | 75 | muHTT |
| | 89 | wtHTT | | 92 | wtHTT |

30 nM

| | | |
|---|---|---|
| WV-905 | 53 | muHTT |
| | 98 | wtHTT |
| WV-911 | 22 | muHTT |
| | 38 | wtHTT |
| WV-917 | 26 | muHTT |
| | 54 | wtHTT |
| WV-931 | 60 | muHTT |
| | 77 | wtHTT |
| WV-937 | 92 | muHTT |
| | 93 | wtHTT |

P13

| | |
|---|---|
| 3.3 nM | 10 nM |

| | | | | | |
|---|---|---|---|---|---|
| WV-904 | 88 | muHTT | WV-904 | 46 | muHTT |
| | 101 | wtHTT | | 74 | wtHTT |
| WV-910 | 73 | muHTT | WV-910 | 29 | muHTT |
| | 80 | wtHTT | | 48 | wtHTT |
| WV-916 | 71 | muHTT | WV-916 | 32 | muHTT |
| | 88 | wtHTT | | 47 | wtHTT |
| WV-930 | 60 | muHTT | WV-930 | 34 | muHTT |
| | 72 | wtHTT | | 48 | wtHTT |
| WV-936 | 111 | muHTT | WV-936 | 67 | muHTT |
| | 98 | wtHTT | | 80 | wtHTT |

30 nM

| | | |
|---|---|---|
| WV-904 | 19 | muHTT |
| | 44 | wtHTT |
| WV-910 | 10 | muHTT |
| | 18 | wtHTT |
| WV-916 | 30 | muHTT |
| | 37 | wtHTT |
| WV-930 | 43 | muHTT |
| | 44 | wtHTT |
| WV-936 | 40 | muHTT |
| | 67 | wtHTT |

Various HTT oligonucleotides were tested in vitro for their ability to decrease the levels of muHTT or wtHTT protein.
HTT oligonucleotide WV-917 was compared in this experiment to a control oligonucleotide, which does not target HTT. Oligonucleotides were tested at 30 nM or 3 nM. Numbers represent quantification of HTT protein (wt or m) expression relative to GAPDH. 1.0 would represent 100% HTT level (0% knockdown) and 0.0 would represent 0% HTT level (100% knock down).

TABLE 5
Activity of certain oligonucleotides.

| | |
|---|---|
| mutant HTT | wt HTT |

| | | | | |
|---|---|---|---|---|
| control 30 nM | 1.06 | 0.94 | 0.96 | 1.04 |
| WV-917 30 nM | 0.18 | 0.19 | 0.71 | 0.75 |
| control 3 nM | 0.78 | 1.22 | 0.96 | 1.04 |
| WV-917 3 nM | 0.25 | 0.17 | 1.14 | 1.08 |

HTT oligonucleotides WV-1510 and WV-1511, which are stereorandom or stereopure, respectively, were tested in vitro for their ability to knock down wild-type (wt) and the mutant (m) HTT corresponding to the SNP.
Results are shown below. Cells were treated with oligonucleotides at concentrations of 0.9 nM, 1.8 nM, 3.8 nM, 7.5 nM, 15 nM, or 30 nM. Numbers represent % of muHTT or wtHTT mRNA left (relative to controls) after treatment with oligonucleotides; numbers are averages of replicate experiments. 1.0 would represent 10000 HTT level (0% knockdown) and 0.0 would represent 0% HTT level (100% knock down).

TABLE 6
Activity of certain oligonucleotides.

| WV-1510 | | mt HTT | | | | wt HTT | | | |
|---|---|---|---|---|---|---|---|---|---|
| 30 | nM | 0.05 | 0.01 | 0.03 | 0.03 | 0.51 | 0.11 | 0.37 | 0.26 |
| 15 | nM | 0.09 | 0.09 | 0.05 | 0.09 | 0.89 | 0.84 | 0.46 | 0.86 |
| 7.5 | nM | 0.03 | 0.01 | 0.21 | 0.23 | 0.30 | 0.18 | 1.57 | 1.57 |
| 3.8 | nM | 0.51 | 0.47 | 0.52 | 0.41 | 1.26 | 1.47 | 1.84 | 1.27 |
| 1.8 | nM | 0.31 | 0.47 | 0.72 | 0.12 | 2.46 | 1.21 | 1.76 | 0.40 |
| 0.9 | nM | 0.80 | 0.65 | 0.76 | | 1.42 | 1.27 | 1.33 | 0.66 |
| WV-1511 | | mt HTT | | | | wt HTT | | | |
| 30 | nM | 0.15 | 0.17 | 0.12 | | 1.41 | 1.35 | 0.93 | 2.16 |
| 15 | nM | | 0.16 | | 0.42 | 2.50 | 1.24 | 2.82 | 2.07 |
| 7.5 | nM | 0.39 | 0.64 | 0.60 | 0.76 | 0.65 | 2.45 | 2.30 | 2.39 |
| 3.8 | nM | 1.04 | | 1.41 | 1.39 | 1.51 | 0.28 | 2.50 | 2.64 |
| 1.8 | nM | 1.76 | 0.26 | 1.44 | 0.29 | 2.55 | 1.80 | 2.11 | 1.96 |
| 0.9 | nM | 0.20 | 0.61 | 0.29 | 1.19 | 1.20 | 0.68 | 0.40 | 1.76 |

Various HTT oligonucleotides comprising SNPs at different positions, and/or different patterns of stereochemistry and/or different 2′-modifications (or patterns thereof) were tested in vitro for their ability to knock down wild-type (wt) and the mutant (in) HTT corresponding to the SNP.
Results are shown below. Cells were treated with oligonucleotides at concentrations of 10 nM or 30 nM.
Numbers represent 0 of muHTT or wtHTT mRNA left (relative to control) after treatment with oligonucleotides; numbers are averages of replicate experiments. 1.0 would represent 100 HTT level (0% knockdown) and 0.0 would represent 0% HTT level (100% knock down). Test was performed for 48 hours. Delta, the difference between the knock-down of MU and WT by a particular oligonucleotide at a particular concentration.

TABLEs 7A
7CB Activity of certain oligonucleotides.

| | 30 nM | | | 10 nM | | |
|---|---|---|---|---|---|---|
| 48 h screen | MU | WT | | MU | WT | |
| Oligonucleotide | HTT | HTT | delta | HTT | HTT | delta |
| WV-993 (control) | 1.00 | 1.01 | 0.00 | 1.00 | 1.00 | 0.00 |
| WV-1077 | 0.91 | 1.07 | 0.17 | 0.98 | 1.08 | 0.10 |
| WV-1078 | 0.32 | 0.47 | 0.15 | 0.69 | 0.83 | 0.15 |
| WV-1079 | 0.78 | 0.77 | −0.01 | 0.87 | 0.88 | 0.01 |
| WV-1080 | 0.67 | 0.63 | −0.04 | 0.82 | 0.93 | 0.11 |
| WV-1081 | 0.81 | 0.72 | −0.09 | 0.83 | 0.97 | 0.13 |
| WV-1082 | 0.80 | 1.00 | 0.20 | 0.91 | 1.06 | 0.15 |
| WV-1083 | 0.65 | 0.93 | 0.29 | 0.88 | 1.04 | 0.16 |
| WV-1084 | 0.69 | 0.90 | 0.21 | 0.96 | 0.95 | −0.01 |
| WV-1508 | 0.66 | 0.88 | 0.23 | 0.86 | 0.96 | 0.10 |
| WV-1509 | 0.31 | 0.83 | 0.52 | 0.63 | 0.98 | 0.35 |
| WV-2023 | 0.38 | 0.91 | 0.53 | 0.66 | 0.90 | 0.24 |
| WV-2024 | 0.59 | 1.18 | 0.60 | 0.90 | 1.10 | 0.20 |
| WV-2025 | 0.65 | 0.99 | 0.34 | 0.83 | 1.02 | 0.19 |
| WV-2026 | 0.41 | 0.74 | 0.32 | 0.79 | 1.06 | 0.27 |
| WV-2027 | 0.50 | 0.90 | 0.39 | 0.80 | 1.01 | 0.20 |
| WV-2028 | 0.45 | 0.79 | 0.34 | 0.79 | 0.87 | 0.09 |
| WV-2029 | 0.56 | 0.77 | 0.21 | 0.87 | 0.97 | 0.10 |
| WV-2030 | 0.28 | 0.66 | 0.38 | 0.71 | 0.80 | 0.09 |
| WV-2031 | 0.30 | 0.58 | 0.28 | 0.58 | 0.80 | 0.22 |
| WV-2032 | 0.28 | 0.53 | 0.26 | 0.58 | 0.70 | 0.12 |
| WV-2033 | 0.31 | 0.58 | 0.27 | 0.69 | 0.76 | 0.07 |
| WV-2034 | 0.31 | 0.51 | 0.19 | 0.68 | 0.97 | 0.29 |
| WV-2035 | 0.22 | 0.52 | 0.30 | 0.54 | 0.82 | 0.28 |
| WV-2036 | 0.31 | 0.48 | 0.17 | 0.62 | 0.73 | 0.12 |
| WV-2037 | 0.23 | 0.48 | 0.25 | 0.55 | 0.69 | 0.14 |
| WV-2038 | 0.23 | 0.61 | 0.38 | 0.49 | 0.81 | 0.32 |
| WV-2039 | 0.36 | 0.60 | 0.24 | 0.71 | 0.84 | 0.13 |
| WV-2040 | 0.30 | 0.51 | 0.21 | 0.60 | 0.76 | 0.17 |
| WV-2041 | 0.26 | 0.63 | 0.36 | 0.63 | 0.82 | 0.19 |
| WV-2042 | 0.27 | 0.53 | 0.26 | 0.66 | 0.86 | 0.21 |
| WV-2043 | 0.53 | 0.64 | 0.12 | 0.91 | 0.90 | −0.01 |
| WV-2044 | 0.47 | 0.61 | 0.14 | 0.81 | 0.88 | 0.07 |
| WV-2045 | 0.47 | 0.78 | 0.30 | 0.76 | 0.89 | 0.14 |
| WV-2046 | 0.60 | 0.91 | 0.31 | 0.80 | 0.87 | 0.07 |
| WV-2047 | 0.50 | 0.57 | 0.07 | 0.81 | 0.77 | −0.04 |
| WV-2048 | 0.58 | 0.63 | 0.05 | 0.79 | 0.78 | −0.02 |
| WV-2049 | 0.55 | 0.69 | 0.13 | 0.87 | 0.86 | −0.01 |
| WV-2050 | 0.62 | 0.77 | 0.15 | 0.98 | 0.98 | 0.01 |
| WV-2051 | 0.50 | 0.49 | 0.00 | 0.80 | 0.72 | −0.08 |
| WV-2052 | 0.47 | 0.55 | 0.08 | 0.79 | 0.71 | −0.09 |
| WV-2053 | 0.55 | 0.59 | 0.03 | 0.85 | 0.79 | −0.06 |
| WV-2054 | 0.49 | 0.56 | 0.07 | 0.75 | 0.78 | 0.03 |
| WV-2055 | 0.54 | 0.63 | 0.09 | 0.90 | 0.96 | 0.06 |
| WV-2056 | 0.50 | 0.64 | 0.14 | 0.75 | 0.71 | −0.04 |
| WV-2057 | 0.51 | 0.81 | 0.30 | 0.71 | 0.95 | 0.24 |
| WV-2058 | 0.71 | 0.89 | 0.18 | 1.03 | 1.10 | 0.07 |
| WV-2059 | 0.68 | 1.01 | 0.33 | 0.91 | 1.00 | 0.08 |
| WV-2060 | 0.43 | 0.81 | 0.38 | 0.72 | 0.94 | 0.22 |
| WV-2061 | 0.46 | 0.96 | 0.50 | 0.79 | 1.04 | 0.25 |
| WV-2062 | 0.53 | 0.83 | 0.30 | 0.82 | 1.03 | 0.21 |
| WV-2063 | 0.61 | 0.92 | 0.30 | 0.85 | 0.99 | 0.14 |
| WV-2064 | 0.33 | 0.77 | 0.44 | 0.67 | 0.99 | 0.32 |
| WV-2065 | 0.30 | 0.65 | 0.35 | 0.64 | 0.92 | 0.28 |
| WV-2066 | 0.25 | 0.56 | 0.31 | 0.69 | 0.79 | 0.10 |
| WV-2067 | 0.25 | 0.59 | 0.34 | 0.56 | 0.91 | 0.35 |
| WV-2068 | 0.22 | 0.52 | 0.31 | 0.52 | 0.78 | 0.26 |
| WV-2069 | 0.12 | 0.33 | 0.22 | 0.36 | 0.69 | 0.33 |
| WV-2070 | 0.20 | 0.52 | 0.32 | 0.58 | 0.84 | 0.26 |
| WV-2071 | 0.18 | 0.35 | 0.17 | 0.40 | 0.65 | 0.25 |
| WV-2072 | 0.17 | 0.46 | 0.29 | 0.35 | 0.73 | 0.38 |
| WV-2073 | 0.36 | 0.66 | 0.30 | 0.69 | 0.95 | 0.26 |
| WV-2074 | 0.20 | 0.46 | 0.26 | 0.60 | 0.78 | 0.18 |
| WV-2075 | 0.18 | 0.51 | 0.33 | 0.46 | 0.85 | 0.39 |
| WV-2076 | 0.18 | 0.69 | 0.50 | 0.51 | 0.84 | 0.32 |
| WV-2077 | 0.85 | 1.04 | 0.19 | 1.07 | 1.20 | 0.13 |
| WV-2078 | 0.71 | 1.00 | 0.29 | 1.02 | 1.14 | 0.12 |
| WV-2079 | 0.62 | 1.01 | 0.39 | 0.83 | 1.15 | 0.32 |
| WV-2080 | 0.59 | 1.04 | 0.45 | 0.95 | 1.09 | 0.15 |
| WV-2081 | 0.81 | 0.91 | 0.10 | 0.99 | 1.11 | 0.12 |
| WV-2082 | 0.83 | 0.95 | 0.11 | 0.96 | 0.92 | −0.04 |
| WV-2083 | 0.76 | 1.03 | 0.27 | 0.98 | 1.13 | 0.15 |
| WV-2084 | 0.86 | 1.13 | 0.28 | 1.03 | 1.01 | −0.01 |
| WV-2085 | 0.69 | 0.70 | 0.01 | 0.89 | 1.03 | 0.14 |
| WV-2087 | 0.78 | 0.87 | 0.10 | 1.00 | 1.07 | 0.08 |
| WV-2088 | 0.98 | 1.02 | 0.03 | 1.11 | 1.15 | 0.04 |
| WV-2089 | 0.91 | 1.06 | 0.15 | 1.10 | 1.14 | 0.03 |
| WV-2090 | 0.73 | 0.72 | −0.01 | 0.93 | 0.89 | −0.04 |

An experiment tested biodistribution of WV-2022 following a single dose and WV-1092 following two biweekly intrathecal doses in cynomolgus monkeys.

TABLE 7A
The set-up for this experiment was:

| | | | | Number of | | |
|---|---|---|---|---|---|---|
| | | | Dose Level | Male | | |
| Group# | Animal# | Test Article | (mg/Animal) | Animals | Dose | Sac date |
| 1 | 12, 24 | WVE-2022 | 4 | 2 | D1 | D4 |
| 2 | 6, 8 | Vehicle (PBS) | 0 | 2 | D1, D15 | D29 |

Sac, sacrifice.
Dose volume=0.5 ml/animal
^{a}Second dose in group 5 was 6 mg
#2 and #4 were swapped from group 1 with #12 and #24
Additional data is not shown.

TABLE 7B
Levels of WV-2022 in Monkey Plasma are shown below, where
numbers indicate level of WV-2022 in plasma (ng/ml).

| | |
|---|---|
| PBS | WV-2022 |

| | | | | | |
|---|---|---|---|---|---|
| Day-1 Pre-dose | 0 | 0 | 0 | 0 | |
| 6 | 0 | 0 | 0 | 799 | 433 |
| 24 | 0 | 0 | 0 | 94 | 121 |
| 96 | 0 | 0 | 0 | 0 | 0 |

### Table 8. Activity of Certain Oligonucleotides.

Various HTT oligonucleotides to SNP rs7685686 were tested in vitro for selectivity for bases at the SNP position: C (wt) or T(mu). Data is shown below.

HTT oligonucleotides WV-2269, WV-2270, WV-2271, WV-2272, WV-2374, and WV-2375 were tested in vitro for ability to knock down wild-type (-WT) and the mutant (-MU) HTT corresponding to the SNP rs7685686. The oligonucleotides differ in chemistry and stereochemistry (or patterns thereof). Oligonucleotides were tested at the described concentrations, and numbers represent percentage of HTT (wt or m) remaining after oligonucleotide treatment. Results from replicate data are shown. Numbers indicate the % of HTT remaining (relative to control) at the indicated oligonucleotide concentrations. 100.0 would represent 100% HTT level (0% knockdown) and 0.0 would represent 0% HTT level (100% knock down). Concentrations are provided as exp 10 in nM. SD, standard deviation. N, number of replicates.

TABLE 9
Activity of certain oligonucleotides.

| Conc. | WV-2269-MU | | | WV-2269-WT | | |
|---|---|---|---|---|---|---|
| Conc. | Mean | SD | N | Mean | SD | N |
| 1.699 | 56.8 | 13.0 | 2 | 66.3 | 5.2 | 2 |
| 1.477 | 62.9 | 11.2 | 2 | 66.0 | 0.6 | 2 |
| 1.176 | 84.0 | 4.4 | 2 | 85.5 | 6.6 | 2 |
| 0.875 | 117.5 | 2.0 | 2 | 90.9 | 2.6 | 2 |
| 0.574 | 100.1 | 3.3 | 2 | 99.6 | 0.3 | 2 |
| 0.273 | 124.7 | 0.9 | 2 | 99.8 | 10.2 | 2 |
| −0.028 | 113.0 | 20.4 | 2 | 96.8 | 8.3 | 2 |
| −0.329 | 83.7 | 18.2 | 2 | 102.0 | 18.7 | 2 |

| | WV-2270-MU | | | WV-2270-WT | | |
|---|---|---|---|---|---|---|
| Conc. | Mean | SD | N | Mean | SD | N |
| 1.699 | 46.2 | 3.1 | 2 | 72.9 | 0.7 | 2 |
| 1.477 | 61.1 | 11.1 | 2 | 76.7 | 4.4 | 2 |
| 1.176 | 82.9 | 10.8 | 2 | 98.4 | 7.4 | 2 |
| 0.875 | 93.3 | 2.4 | 2 | 101.4 | 4.5 | 2 |
| 0.574 | 104.2 | 5.4 | 2 | 105.6 | 0.8 | 2 |
| 0.273 | 112.9 | 4.8 | 2 | 96.7 | 2.0 | 2 |
| −0.028 | 112.5 | 1.5 | 2 | 95.4 | 3.8 | 2 |
| −0.329 | 103.3 | 2.0 | 2 | 99.2 | 9.6 | 2 |

| | WV-2271-MU | | | WV-2271-WT | | |
|---|---|---|---|---|---|---|
| Conc. | Mean | SD | N | Mean | SD | N |
| 1.699 | 40.4 | 12.8 | 2 | 77.1 | 9.0 | 2 |
| 1.477 | 47.4 | 9.8 | 2 | 86.5 | 8.7 | 2 |
| 1.176 | 67.5 | 2.5 | 2 | 89.6 | 1.9 | 2 |
| 0.875 | 76.5 | 0.5 | 2 | 95.0 | 3.0 | 2 |
| 0.574 | 90.3 | 1.9 | 2 | 108.3 | 10.6 | 2 |
| 0.273 | 112.0 | 10.9 | 2 | 99.0 | 5.2 | 2 |
| −0.028 | 110.1 | 0.5 | 2 | 98.4 | 4.2 | 2 |
| −0.329 | 99.2 | 0.4 | 2 | 98.5 | 1.2 | 2 |
| | WV-2272-MU | | | WV-2272-WT | | |
| Conc. | Mean | SD | N | Mean | SD | N |
| 1.699 | 59.5 | 2.4 | 2 | 87.1 | 5.3 | 2 |
| 1.477 | 65.9 | 15.9 | 2 | 91.2 | 7.1 | 2 |
| 1.176 | 83.0 | 4.2 | 2 | 99.5 | 11.5 | 2 |
| 0.875 | 100.8 | 0.1 | 2 | 93.8 | 2.1 | 2 |
| 0.574 | 111.6 | 5.8 | 2 | 102.5 | 1.9 | 2 |
| 0.273 | 116.6 | 8.6 | 2 | 98.9 | 4.9 | 2 |
| −0.028 | 123.9 | 0.4 | 2 | 107.0 | 11.9 | 2 |
| −0.329 | 94.5 | 13.3 | 2 | 108.1 | 7.7 | 2 |
| | WV-2374-MU | | | WV-2374-WT | | |
| Conc. | Mean | SD | N | Mean | SD | N |
| 1.699 | 36.6 | 4.9 | 2 | 69.5 | 8.0 | 2 |
| 1.477 | 56.9 | 6.9 | 2 | 74.1 | 11.3 | 2 |
| 1.176 | 80.3 | 9.7 | 2 | 92.8 | 6.9 | 2 |
| 0.875 | 92.3 | 10.8 | 2 | 96.9 | 2.1 | 2 |
| 0.574 | 103.0 | 7.7 | 2 | 110.9 | 0.5 | 2 |
| 0.273 | 110.2 | 1.2 | 2 | 97.3 | 10.7 | 2 |
| −0.028 | 110.4 | 7.6 | 2 | 94.5 | 6.2 | 2 |
| −0.329 | 102.7 | 4.6 | 2 | 96.9 | 5.1 | 2 |
| | WV-2375-MU | | | WV-2375-WT | | |
| Conc. | Mean | SD | N | Mean | SD | N |
| 1.699 | 54.2 | 1.6 | 2 | 83.4 | 9.1 | 2 |
| 1.477 | 67.9 | 16.7 | 2 | 88.0 | 11.2 | 2 |
| 1.176 | 78.8 | 2.3 | 2 | 94.9 | 4.8 | 2 |
| 0.875 | 105.6 | 6.1 | 2 | 96.9 | 2.4 | 2 |
| 0.574 | 93.1 | 4.8 | 2 | 101.9 | 7.1 | 2 |
| 0.273 | 106.5 | 8.8 | 2 | 97.1 | 3.9 | 2 |
| −0.028 | 101.8 | 5.4 | 2 | 90.8 | 3.5 | 2 |
| −0.329 | 87.3 | 7.1 | 2 | 109.2 | 4.5 | 2 |

HTT oligonucleotide WV-3857 was also tested for its ability to knockdown wt and mutant HTT. Concentrations are provided as exp 10 in nM.
The results are shown below. The numbers represent the HTT (wt or mu) levels relative to controls, wherein 1.0 would represent 100.0% HTT level (0% knockdown) and 0.0 would represent 0% HTT level (100% knockdown).

TABLE 10
Activity of certain oligonucleotides.

| Concentration | WT HTT | | | Mu HTT | | |
|---|---|---|---|---|---|---|
| 1.39794 | 0.645 | 0.605 | 0.585 | 0.271 | 0.211 | 0.180 |
| 1.09691 | 0.676 | 0.819 | 0.755 | 0.427 | 0.375 | 0.440 |
| 0.787106 | 0.769 | 0.801 | 0.852 | 0.553 | 0.560 | 0.679 |
| 0.49485 | 0.874 | 0.869 | 0.827 | 0.608 | 0.763 | 0.696 |
| 0.19382 | 0.861 | 1.048 | 0.973 | 0.724 | 0.770 | 0.737 |
| −0.10721 | 0.845 | 0.969 | 0.934 | 0.808 | 1.035 | 0.989 |
| −0.40824 | 1.013 | 1.084 | 1.178 | 0.773 | 0.679 | 0.922 |
| −0.70927 | 0.980 | 1.330 | 1.069 | 0.930 | 0.757 | 1.087 |

Various HTT oligonucleotides were tested in vitro for their ability to knockdown wt and mutant HTT. Concentrations are provided as exp 10 in nM.
Various HTT oligonucleotides target rs2530595: WV-2589, WV-2590, WV-2591, WV-2592, WV-2593, WV-2594, WV-2595, WV-2596, WV-2605, WV-2606, WV-2607, WV-2608, WV-2609, WV-2610, WV-2611, WV-2612.
Various HTT oligonucleotides target rs rs362331: WV-2597, WV-2598, WV-2598, WV-2599, WV-2600, WV-2600, WV-2601, WV-2601, WV-2602, WV-2603, WV-2604, WV-2613, WV-2614, WV-2615, WV-2615, WV-2616, WV-2616, WV-2617, WV-2618, WV-2619, WV-2620.
Cells used had been evaluated for SNPs rs362331 (331), rs2530595 (595), and rs113407847 (847): Tri.SNP 331 T 595:T 847:G
Numbers indicate the % of HTT remaining (relative to control) at the indicated oligonucleotide concentrations. 100.0 would represent 100% HTT mRNA remaining (0.0% knockdown); and 0.0 will represent 0.00% HTT mRNA remaining (100.0% knockdown).

TABLE 11
Activity of certain oligonucleotides.

| | TriSNP 331: T 595: T 847: G | | TriSNP 331: C 595: C 847: A | |
|---|---|---|---|---|
| WV-2589 | | | | |
| 1.699 | 60.56 | 59.99 | 73.13 | 73.13 |
| 1.477 | 84.59 | 75.72 | 76.43 | 97.70 |
| 1.176 | 104.51 | 102.05 | 82.63 | 90.81 |
| 0.875 | 106.90 | 97.63 | 83.43 | 115.51 |
| 0.574 | 116.45 | 109.91 | 102.60 | 115.75 |
| 0.273 | 104.83 | 104.19 | 106.96 | 89.78 |
| −0.028 | 76.39 | 98.88 | 110.51 | 87.01 |
| −0.329 | 103.02 | 98.52 | 99.91 | 91.42 |
| WV-2605 | | | | |
| 1.699 | 76.21 | 75.49 | 102.01 | 120.60 |
| 1.477 | 109.11 | 84.23 | 100.76 | 104.90 |
| 1.176 | 95.17 | 101.40 | 86.21 | 91.18 |
| 0.875 | 103.09 | 111.48 | 88.48 | 102.57 |
| 0.574 | 101.36 | 94.18 | 105.97 | 92.48 |
| 0.273 | 96.35 | 90.17 | 106.74 | 101.16 |
| −0.028 | 92.36 | 96.42 | 111.91 | 90.02 |
| −0.329 | 90.06 | 92.04 | 106.05 | 104.61 |
| WV-2590 | | | | |
| 1.699 | 56.81 | 48.92 | 69.59 | 69.59 |
| 1.477 | 79.06 | 68.62 | 72.88 | 80.95 |
| 1.176 | 95.51 | 100.01 | 84.90 | 86.38 |
| 0.875 | 85.78 | 96.42 | 97.38 | 119.00 |
| 0.574 | 97.20 | 105.62 | 117.26 | 103.56 |
| 0.273 | 106.54 | 102.42 | 104.77 | 92.00 |
| −0.028 | 87.26 | 90.98 | 115.59 | 115.34 |
| −0.329 | 85.36 | 86.55 | 109.06 | 99.29 |
| WV-2606 | | | | |
| 1.699 | 60.56 | 59.99 | 73.13 | 73.13 |
| 1.477 | 84.59 | 75.72 | 76.43 | 97.70 |
| 1.176 | 104.51 | 102.05 | 82.63 | 90.81 |
| 0.875 | 106.90 | 97.63 | 83.43 | 115.51 |
| 0.574 | 116.45 | 109.91 | 102.60 | 115.75 |
| 0.273 | 104.83 | 104.19 | 106.96 | 89.78 |
| −0.028 | 76.39 | 98.88 | 110.51 | 87.01 |
| −0.329 | 103.02 | 98.52 | 99.91 | 91.42 |
| WV-2591 | | | | |
| 1.699 | 61.29 | 52.18 | 74.59 | 74.59 |
| 1.477 | 73.17 | 67.19 | 77.04 | 74.37 |
| 1.176 | 92.25 | 77.15 | 74.94 | 96.19 |
| 0.875 | 83.18 | 89.29 | 93.37 | 111.81 |
| 0.574 | 94.98 | 95.11 | 110.53 | 92.41 |
| 0.273 | 93.49 | 98.46 | 115.82 | 111.46 |
| −0.028 | 87.44 | 91.42 | 108.07 | 97.49 |
| −0.329 | 93.91 | 89.80 | 109.29 | 96.15 |
| WV-2607 | | | | |
| 1.699 | 50.89 | 43.34 | 78.77 | 75.20 |
| 1.477 | 67.55 | 68.91 | 84.48 | 84.48 |
| 1.176 | 88.89 | 82.88 | 82.91 | 91.91 |
| 0.875 | 87.54 | 84.13 | 88.23 | 104.07 |
| 0.574 | 82.06 | 86.50 | 104.34 | 119.11 |
| 0.273 | 97.43 | 93.86 | 111.33 | 124.91 |
| −0.028 | 89.06 | 90.45 | 110.25 | 89.83 |
| −0.329 | 100.90 | 92.67 | 95.72 | 146.11 |
| WV-2592 | | | | |
| 1.699 | 44.51 | 36.30 | 56.48 | 56.48 |
| 1.477 | 60.09 | 60.65 | 71.51 | 78.01 |
| 1.176 | 87.30 | 81.29 | 73.44 | 105.91 |
| 0.875 | 91.29 | 86.41 | 97.44 | 106.35 |
| 0.574 | 88.15 | 86.44 | 97.40 | 99.54 |
| 0.273 | 88.41 | 88.73 | 102.75 | 80.53 |
| −0.028 | 82.26 | 90.69 | 108.84 | 84.08 |
| −0.329 | 99.81 | 92.76 | 100.74 | 95.08 |
| WV-2608 | | | | |
| 1.699 | 55.18 | 55.70 | 77.97 | 77.97 |
| 1.477 | 72.53 | 73.26 | 89.00 | 78.37 |
| 1.176 | 97.42 | 83.57 | 89.61 | 81.92 |
| 0.875 | 87.82 | 85.78 | 92.52 | 95.22 |
| 0.574 | 91.86 | 88.07 | 104.86 | 98.64 |
| 0.273 | 99.14 | 87.70 | 111.19 | 121.03 |
| −0.028 | 103.30 | 95.09 | 112.63 | 83.34 |
| −0.329 | 101.77 | 93.71 | 93.50 | 114.82 |
| WV-2593 | | | | |
| 1.699 | 59.90 | 51.49 | 67.35 | 69.80 |
| 1.477 | 69.11 | 60.53 | 90.63 | 90.74 |
| 1.176 | 94.30 | 82.20 | 76.32 | 113.94 |
| 0.875 | 83.40 | 72.28 | 98.13 | 104.51 |
| 0.574 | 76.72 | 82.39 | 107.29 | 119.32 |
| 0.273 | 99.77 | 84.51 | 103.30 | 114.64 |
| −0.028 | 90.82 | 81.20 | 104.86 | 104.53 |
| −0.329 | 89.82 | 106.14 | 103.22 | 102.25 |
| WV-2609 | | | | |
| 1.699 | 41.18 | 36.39 | 83.24 | 81.23 |
| 1.477 | 49.08 | 54.91 | 100.39 | 103.27 |
| 1.176 | 70.48 | 71.94 | 84.64 | 109.62 |
| 0.875 | 87.46 | 86.62 | 105.04 | 110.44 |
| 0.574 | 81.77 | 90.92 | 102.49 | 118.53 |
| 0.273 | 88.78 | 93.03 | 98.52 | 113.30 |
| −0.028 | 93.91 | 93.51 | 106.84 | 104.27 |
| −0.329 | 93.09 | 100.58 | 93.69 | 94.10 |
| WV-2594 | | | | |
| 1.699 | 36.68 | 42.41 | 68.79 | 70.94 |
| 1.477 | 53.17 | 56.17 | 92.07 | 81.30 |
| 1.176 | 71.57 | 67.29 | 78.70 | 108.31 |
| 0.875 | 82.03 | 84.98 | 91.93 | 99.56 |
| 0.574 | 80.78 | 91.18 | 106.80 | 120.13 |
| 0.273 | 85.25 | 96.09 | 109.01 | 108.69 |
| −0.028 | 94.03 | 97.14 | 114.05 | 108.20 |
| −0.329 | 87.26 | 99.36 | 97.12 | 94.69 |
| WV-2610 | | | | |
| 1.699 | 59.90 | 51.49 | 67.35 | 69.80 |
| 1.477 | 69.11 | 60.53 | 90.63 | 90.74 |
| 1.176 | 94.30 | 82.20 | 76.32 | 113.94 |
| 0.875 | 83.40 | 72.28 | 98.13 | 104.51 |
| 0.574 | 76.72 | 82.39 | 107.29 | 119.32 |
| 0.273 | 99.77 | 84.51 | 103.30 | 114.64 |
| −0.028 | 90.82 | 81.20 | 104.86 | 104.53 |
| −0.329 | 89.82 | 106.14 | 103.22 | 102.25 |
| WV-2595 | | | | |
| 1.699 | 48.63 | 55.00 | 108.23 | 94.01 |
| 1.477 | 56.15 | 60.99 | 108.71 | 102.72 |
| 1.176 | 59.74 | 63.60 | 88.07 | 113.94 |
| 0.875 | 69.96 | 70.36 | 94.91 | 99.53 |
| 0.574 | 77.27 | 87.30 | 93.34 | 96.69 |
| 0.273 | 82.53 | 92.33 | 93.38 | 105.64 |
| −0.028 | 86.03 | 88.27 | 97.01 | 105.04 |
| −0.329 | 86.78 | 109.44 | 96.60 | 98.33 |
| WV-2611 | | | | |
| 1.699 | 9.53 | 12.17 | 16.24 | 19.22 |
| 1.477 | 16.93 | 19.15 | 24.19 | 27.85 |
| 1.176 | 28.74 | 28.40 | 32.35 | 43.92 |
| 0.875 | 52.36 | 48.97 | 72.01 | 61.71 |
| 0.574 | 62.13 | 68.97 | 75.22 | 85.90 |
| 0.273 | 73.42 | 80.56 | 92.76 | 97.03 |
| −0.028 | 81.74 | 90.76 | 99.53 | 93.82 |
| −0.329 | 82.47 | 95.95 | 96.76 | 96.52 |
| WV-2596 | | | | |
| 1.699 | 59.84 | 74.70 | 70.94 | 76.29 |
| 1.477 | 84.66 | 75.20 | 79.80 | 81.33 |
| 1.176 | 89.32 | 78.25 | 83.24 | 103.68 |
| 0.875 | 91.58 | 97.35 | 104.68 | 92.25 |
| 0.574 | 91.47 | 96.52 | 96.40 | 104.49 |
| 0.273 | 89.55 | 92.16 | 96.23 | 107.24 |
| −0.028 | 91.77 | 95.93 | 90.13 | 101.39 |
| −0.329 | 79.68 | 110.06 | 102.78 | 92.17 |
| WV-2612 | | | | |
| 1.699 | 10.94 | 10.86 | 12.82 | 16.33 |
| 1.477 | 17.06 | 17.76 | 21.63 | 23.24 |
| 1.176 | 32.61 | 35.17 | 33.77 | 43.61 |
| 0.875 | 51.64 | 54.88 | 64.00 | 65.67 |
| 0.574 | 69.62 | 77.36 | 75.46 | 82.10 |
| 0.273 | 74.50 | 84.34 | 89.20 | 95.70 |
| −0.028 | 83.05 | 92.02 | 94.17 | 95.84 |
| −0.329 | 88.60 | 98.41 | 95.14 | 94.00 |
| WV-2597 | | | | |
| 1.699 | 29.61 | 30.21 | 66.48 | 68.22 |
| 1.477 | 37.74 | 32.36 | 76.24 | 78.00 |
| 1.176 | 57.70 | 50.67 | 99.88 | 75.94 |
| 0.875 | 89.55 | 71.98 | 76.70 | 86.04 |
| 0.574 | 88.07 | 92.17 | 82.68 | 101.53 |
| 0.273 | 96.83 | 80.80 | 103.41 | 88.74 |
| −0.028 | 97.15 | 95.68 | 87.47 | 103.65 |
| −0.329 | 98.82 | 94.29 | 89.41 | 96.63 |
| WV-2613 | | | | |
| 1.699 | 28.11 | 24.04 | 42.67 | 48.74 |
| 1.477 | 28.67 | 25.07 | 45.16 | 53.34 |
| 1.176 | 40.17 | 35.85 | 68.59 | 55.75 |
| 0.875 | 60.41 | 48.93 | 79.53 | 73.94 |
| 0.574 | 78.22 | 82.17 | 78.28 | 103.33 |
| 0.273 | 90.92 | 72.02 | 96.62 | 79.34 |
| −0.028 | 84.94 | 98.26 | 98.81 | 111.40 |
| −0.329 | 98.48 | 89.94 | 89.55 | 92.90 |
| WV-2598 | | | | |
| 1.699 | 39.34 | 34.94 | 70.81 | 83.66 |
| 1.477 | 41.96 | 39.88 | 78.59 | 93.32 |
| 1.176 | 64.02 | 57.60 | 105.86 | 90.84 |
| 0.875 | 82.66 | 69.60 | 99.33 | 89.31 |
| 0.574 | 81.63 | 97.86 | 101.36 | 112.32 |
| 0.273 | 95.10 | 76.28 | 98.98 | 92.96 |
| −0.028 | 87.89 | 91.96 | 103.25 | 106.63 |
| −0.329 | 97.88 | 95.16 | 89.31 | 100.06 |
| WV-2671 | | | | |
| 1.699 | 49.62 | 51.71 | 106.50 | 82.29 |
| 1.477 | 46.61 | 53.08 | 83.01 | 81.23 |
| 1.176 | 70.37 | 57.12 | 96.83 | 101.03 |
| 0.875 | 70.81 | 77.25 | 98.87 | 96.44 |
| 0.574 | 92.54 | 108.12 | 89.48 | 103.29 |
| 0.273 | 89.54 | 108.00 | 100.91 | 98.91 |
| −0.028 | 89.68 | 99.98 | 95.94 | 89.76 |
| −0.329 | 101.96 | 105.05 | 92.16 | 94.46 |
| WV-2672 | | | | |
| 1.699 | 50.57 | 62.40 | 64.71 | 71.10 |
| 1.477 | 54.62 | 58.74 | 67.04 | 63.64 |
| 1.176 | 68.45 | 69.50 | 81.41 | 83.29 |
| 0.875 | 71.05 | 81.78 | 91.61 | 94.06 |
| 0.574 | 88.41 | 114.02 | 91.71 | 79.34 |
| 0.273 | 85.37 | 112.41 | 97.78 | 97.11 |
| −0.028 | 95.56 | 101.84 | 97.08 | 78.91 |
| −0.329 | 99.60 | 117.61 | 88.58 | 87.73 |
| WV-2673 | | | | |
| 1.699 | 70.63 | 59.47 | 90.32 | 85.01 |
| 1.477 | 57.99 | 87.49 | 93.47 | 82.31 |
| 1.176 | 84.00 | 78.30 | 104.86 | 96.15 |
| 0.875 | 67.75 | 87.55 | 88.28 | 96.45 |
| 0.574 | 96.14 | 107.16 | 79.89 | 95.96 |
| 0.273 | 83.56 | 95.11 | 94.48 | 90.48 |
| −0.028 | 102.34 | 101.72 | 84.57 | 105.47 |
| −0.329 | 96.35 | 101.60 | 86.24 | 88.06 |
| WV-2614 | | | | |
| 1.699 | 29.61 | 30.21 | 68.22 | 66.48 |
| 1.477 | 37.74 | 32.36 | 78.00 | 76.24 |
| 1.176 | 57.70 | 50.67 | 75.94 | 99.88 |
| 0.875 | 89.55 | 71.98 | 86.04 | 76.70 |
| 0.574 | 88.07 | 92.17 | 101.53 | 82.68 |
| 0.273 | 96.83 | 80.80 | 88.74 | 103.41 |
| −0.028 | 97.15 | 95.68 | 103.65 | 87.47 |
| −0.329 | 98.82 | 94.29 | 96.63 | 89.41 |
| WV-2599 | | | | |
| 1.699 | 48.99 | 46.90 | 85.01 | 96.26 |
| 1.477 | 63.67 | 55.92 | 92.54 | 107.39 |
| 1.176 | 72.10 | 70.59 | 121.06 | 96.16 |
| 0.875 | 78.65 | 73.28 | 94.01 | 92.46 |
| 0.574 | 88.04 | 96.90 | 106.27 | 108.41 |
| 0.273 | 92.37 | 78.57 | 103.69 | 93.02 |
| −0.028 | 90.84 | 92.38 | 95.92 | 116.47 |
| −0.329 | 93.64 | 105.04 | 101.18 | 104.15 |
| WV-2615 | | | | |
| 1.699 | 38.37 | 32.70 | 62.45 | 74.37 |
| 1.477 | 33.80 | 28.14 | 69.96 | 75.71 |
| 1.176 | 31.82 | 33.22 | 86.23 | 68.08 |
| 0.875 | 50.64 | 42.96 | 80.20 | 80.35 |
| 0.574 | 64.74 | 69.82 | 95.61 | 101.13 |
| 0.273 | 81.21 | 73.64 | 96.84 | 87.40 |
| −0.028 | 84.66 | 85.23 | 105.03 | 104.22 |
| −0.329 | 90.15 | 86.99 | 102.54 | 105.95 |
| WV-2600 | | | | |
| 1.699 | 45.55 | 38.85 | 69.41 | 77.04 |
| 1.477 | 52.56 | 45.58 | 82.18 | 92.46 |
| 1.176 | 69.40 | 63.85 | 106.85 | 85.90 |
| 0.875 | 83.92 | 60.75 | 95.24 | 97.62 |
| 0.574 | 89.75 | 83.36 | 89.90 | 96.00 |
| 0.273 | 89.44 | 78.32 | 94.69 | 86.53 |
| −0.028 | 94.51 | 89.22 | 95.23 | 104.24 |
| −0.329 | 87.39 | 93.62 | 93.46 | 107.66 |
| WV-2616 | | | | |
| 1.699 | 13.21 | 12.74 | 35.53 | 34.76 |
| 1.477 | 15.55 | 12.90 | 40.87 | 43.14 |
| 1.176 | 24.38 | 22.49 | 68.20 | 54.00 |
| 0.875 | 43.08 | 35.88 | 74.25 | 87.86 |
| 0.574 | 61.18 | 64.53 | 90.47 | 93.71 |
| 0.273 | 83.68 | 62.88 | 97.02 | 86.72 |
| −0.028 | 90.85 | 80.56 | 97.30 | 104.95 |
| −0.329 | 83.31 | 92.60 | 104.41 | 102.71 |
| WV-2601 | | | | |
| 1.699 | 34.00 | 34.55 | 85.74 | 89.57 |
| 1.477 | 43.98 | 30.94 | 88.05 | 102.32 |
| 1.176 | 58.81 | 41.52 | 93.01 | 90.59 |
| 0.875 | 58.64 | 50.11 | 90.45 | 103.00 |
| 0.574 | 68.58 | 67.67 | 72.86 | 106.45 |
| 0.273 | 84.80 | 81.87 | 96.72 | 111.30 |
| −0.028 | 86.59 | 83.35 | 101.14 | 97.79 |
| −0.329 | 92.40 | 91.95 | 107.80 | 104.50 |
| WV-2617 | | | | |
| 1.699 | 10.10 | 11.21 | 27.20 | 34.81 |
| 1.477 | 8.66 | 9.22 | 33.51 | 39.87 |
| 1.176 | 13.76 | 14.25 | 55.48 | 55.42 |
| 0.875 | 26.60 | 21.64 | 68.51 | 68.76 |
| 0.574 | 30.07 | 36.31 | 70.72 | 83.75 |
| 0.273 | 50.23 | 65.22 | 87.57 | 90.34 |
| −0.028 | 64.69 | 63.27 | 105.89 | 104.40 |
| −0.329 | 67.17 | 66.15 | 102.31 | 97.77 |
| WV-2602 | | | | |
| 1.699 | 39.21 | 37.73 | 95.85 | 115.93 |
| 1.477 | 33.57 | 34.08 | 97.00 | 97.48 |
| 1.176 | 44.51 | 42.51 | 113.00 | 108.53 |
| 0.875 | 57.54 | 58.61 | 97.96 | 103.49 |
| 0.574 | 75.51 | 84.44 | 93.08 | 99.80 |
| 0.273 | 83.25 | 105.30 | 98.52 | 106.48 |
| −0.028 | 87.46 | 83.72 | 102.60 | 103.89 |
| −0.329 | 94.42 | 82.39 | 112.36 | 92.34 |
| WV-2618 | | | | |
| 1.699 | 34.00 | 34.55 | 85.74 | 89.57 |
| 1.477 | 43.98 | 30.94 | 88.05 | 102.32 |
| 1.176 | 58.81 | 41.52 | 93.01 | 90.59 |
| 0.875 | 58.64 | 50.11 | 90.45 | 103.00 |
| 0.574 | 68.58 | 67.67 | 72.86 | 106.45 |
| 0.273 | 84.80 | 81.87 | 96.72 | 111.30 |
| −0.028 | 86.59 | 83.35 | 101.14 | 97.79 |
| −0.329 | 92.40 | 91.95 | 107.80 | 104.50 |
| WV-2603 | | | | |
| 1.699 | 7.82 | 8.30 | 79.44 | 90.25 |
| 1.477 | 10.59 | 12.05 | 77.75 | 91.12 |
| 1.176 | 20.99 | 19.76 | 96.91 | 99.41 |
| 0.875 | 34.92 | 36.01 | 95.25 | 99.75 |
| 0.574 | 47.81 | 51.36 | 83.10 | 105.34 |
| 0.273 | 60.78 | 77.76 | 97.23 | 102.94 |
| −0.028 | 74.20 | 61.96 | 97.13 | 106.41 |
| −0.329 | 76.10 | 80.38 | 112.13 | 100.07 |
| WV-2619 | | | | |
| 1.699 | 7.95 | 7.94 | 52.37 | 59.82 |
| 1.477 | 13.51 | 15.08 | 60.09 | 70.38 |
| 1.176 | 25.38 | 25.75 | 85.54 | 79.89 |
| 0.875 | 39.80 | 43.01 | 81.18 | 89.62 |
| 0.574 | 42.84 | 59.52 | 79.40 | 94.91 |
| 0.273 | 61.65 | 74.72 | 96.87 | 88.17 |
| −0.028 | 79.32 | 59.57 | 93.85 | 104.46 |
| −0.329 | 84.73 | 87.90 | 101.07 | 101.08 |
| WV-2604 | | | | |
| 1.699 | 53.99 | 55.77 | 86.56 | 96.69 |
| 1.477 | 60.35 | 70.59 | 85.90 | 99.37 |
| 1.176 | 66.00 | 75.22 | 98.36 | 102.85 |
| 0.875 | 76.26 | 79.46 | 104.05 | 100.25 |
| 0.574 | 84.45 | 94.61 | 87.16 | 98.54 |
| 0.273 | 88.76 | 102.45 | 105.80 | 98.23 |
| −0.028 | 86.38 | 92.63 | 98.06 | 101.54 |
| −0.329 | 92.53 | 98.71 | 115.03 | 103.91 |
| WV-2620 | | | | |
| 1.699 | 14.72 | 12.77 | 29.52 | 36.45 |
| 1.477 | 13.29 | 12.38 | 39.69 | 39.27 |
| 1.176 | 14.82 | 13.71 | 50.34 | 48.69 |
| 0.875 | 30.43 | 29.47 | 72.48 | 69.50 |
| 0.574 | 42.09 | 49.31 | 74.67 | 79.53 |
| 0.273 | 57.10 | 62.24 | 91.08 | 91.55 |
| −0.028 | 85.77 | 84.91 | 101.04 | 100.37 |
| −0.329 | 92.90 | 85.26 | 98.27 | 94.08 |
| WV-2598 | | | | |
| 1.699 | 39.34 | 34.94 | 70.81 | 83.66 |
| 1.477 | 41.96 | 39.88 | 78.59 | 93.32 |
| 1.176 | 64.02 | 57.60 | 105.86 | 90.84 |
| 0.875 | 82.66 | 69.60 | 99.33 | 89.31 |
| 0.574 | 81.63 | 97.86 | 101.36 | 112.32 |
| 0.273 | 95.10 | 76.28 | 98.98 | 92.96 |
| −0.028 | 87.89 | 91.96 | 103.25 | 106.63 |
| −0.329 | 97.88 | 95.16 | 89.31 | 100.06 |
| WV-2600 | | | | |
| 1.699 | 45.55 | 38.85 | 69.41 | 77.04 |
| 1.477 | 52.56 | 45.58 | 82.18 | 92.46 |
| 1.176 | 69.40 | 63.85 | 106.85 | 85.90 |
| 0.875 | 83.92 | 60.75 | 95.24 | 97.62 |
| 0.574 | 89.75 | 83.36 | 89.90 | 96.00 |
| 0.273 | 89.44 | 78.32 | 94.69 | 86.53 |
| −0.028 | 94.51 | 89.22 | 95.23 | 104.24 |
| −0.329 | 87.39 | 93.62 | 93.46 | 107.66 |
| WV-2615 | | | | |
| 1.699 | 38.37 | 32.70 | 62.45 | 74.37 |
| 1.477 | 33.80 | 28.14 | 69.96 | 75.71 |
| 1.176 | 31.82 | 33.22 | 86.23 | 68.08 |
| 0.875 | 50.64 | 42.96 | 80.20 | 80.35 |
| 0.574 | 64.74 | 69.82 | 95.61 | 101.13 |
| 0.273 | 81.21 | 73.64 | 96.84 | 87.40 |
| −0.028 | 84.66 | 85.23 | 105.03 | 104.22 |
| −0.329 | 90.15 | 86.99 | 102.54 | 105.95 |
| WV-2616 | | | | |
| 1.699 | 13.21 | 12.74 | 35.53 | 34.76 |
| 1.477 | 15.55 | 12.90 | 40.87 | 43.14 |
| 1.176 | 24.38 | 22.49 | 68.20 | 54.00 |
| 0.875 | 43.08 | 35.88 | 74.25 | 87.86 |
| 0.574 | 61.18 | 64.53 | 90.47 | 93.71 |
| 0.273 | 83.68 | 62.88 | 97.02 | 86.72 |
| −0.028 | 90.85 | 80.56 | 97.30 | 104.95 |
| −0.329 | 83.31 | 92.60 | 104.41 | 102.71 |
| WV-2601 | | | | |
| 1.699 | 34.00 | 34.55 | 85.74 | 89.57 |
| 1.477 | 43.98 | 30.94 | 88.05 | 102.32 |
| 1.176 | 58.81 | 41.52 | 93.01 | 90.59 |
| 0.875 | 58.64 | 50.11 | 90.45 | 103.00 |
| 0.574 | 68.58 | 67.67 | 72.86 | 106.45 |
| 0.273 | 84.80 | 81.87 | 96.72 | 111.30 |
| −0.028 | 86.59 | 83.35 | 101.14 | 97.79 |
| −0.329 | 92.40 | 91.95 | 107.80 | 104.50 |
| WV-2674 | | | | |
| 1.699 | 54.66 | 55.27 | 61.39 | 52.31 |
| 1.477 | 54.48 | 55.82 | 67.66 | 63.47 |
| 1.176 | 70.20 | 61.27 | 80.70 | 83.14 |
| 0.875 | 69.15 | 78.41 | 85.98 | 87.31 |
| 0.574 | 90.30 | 105.17 | 85.06 | 86.20 |
| 0.273 | 85.35 | 96.28 | 98.56 | 91.76 |
| −0.028 | 86.98 | 98.99 | 89.18 | 97.60 |
| −0.329 | 100.95 | 111.21 | 85.39 | 95.54 |
| WV-2675 | | | | |
| 1.699 | 79.48 | 80.19 | 109.27 | 101.57 |
| 1.477 | 79.64 | 102.82 | 113.63 | 116.68 |
| 1.176 | 102.89 | 84.65 | 91.85 | 111.81 |
| 0.875 | 77.95 | 91.55 | 95.08 | 94.81 |
| 0.574 | 103.46 | 99.30 | 90.31 | 95.32 |
| 0.273 | 84.48 | 93.60 | 97.34 | 96.16 |
| −0.028 | 95.72 | 104.65 | 94.76 | 96.77 |
| −0.329 | 107.30 | 117.95 | 94.83 | 93.27 |

Additional HTT oligonucleotides were screened for their ability to knockdown mutant and wild-type HTT.
Two primary fibroblast cell lines, designated herein as ND33947 (sometimes designated ND33947) and GM01169 (sometimes designated GM01147), were chosen based on initial sequencing and phasing data; these are heterozygous for both rs362307 and rs362331 SNPs. Cells were electroporated with control and test oligonucleotides targeting rs362307 or rs362331 SNPs. Concentrations used were: 2.5 μM and M; samples were collected after 48 hours and HTT knockdown was assessed via Taqman. NGS (Next Generation Sequencing) was used to determine allele specificity.
Some of the tested HTT oligonucleotides (e.g., WV-4241, WV-4242, WV-4243, and WV-4244) represent shortened versions of other HTT oligonucleotides; these shortened oligonucleotides also represent metabolites of the longer oligonucleotides.
Numbers indicate the % of HTT remaining (relative to control) at the indicated oligonucleotide concentrations. Data was normalized to controls; 100.0 would represent 10000 wt or mutant HTT level (0% knockdown); and 0.0 would represent 0.0% HTT level (100.0% knockdown).
wt C or mutant T indicate the isoform of rs362307.

TABLE 12
Activity of certain oligonucleotides.

| | |
|---|---|
| wt C | mut T |

ND33947 cells, testing rs362307 SNP 2.5 uM Normalized

| | | | | | | |
|---|---|---|---|---|---|---|
| PBS | 125.1 | 86.5 | 88.4 | 131.6 | 89.2 | 99.4 |
| WV-993 | 71.3 | 68.9 | 82.1 | 75.9 | 78.3 | 90.2 |
| WV-1510 | 59.7 | 67.8 | 75.7 | 55.8 | 67.2 | 83.1 |
| WV-2603 | 61.7 | 68.6 | 104.8 | 59.3 | 64.7 | 102.7 |
| WV-4241 | 66.5 | 66.4 | 64.6 | 72.3 | 68.4 | 68.8 |
| WV-4242 | 84.7 | 103.6 | 118.6 | 90.1 | 111.4 | 121.0 |
| WV-4243 | 62.2 | 75.2 | 88.6 | 66.8 | 76.0 | 88.6 |
| WV-4244 | 130.1 | 142.5 | 94.5 | 138.5 | 151.6 | 93.6 |
| WV-8706 | 138.5 | 148.9 | 199.0 | 80.0 | 83.7 | 99.5 |
| WV-8707 | 79.7 | 121.0 | 105.3 | 33.2 | 56.0 | 52.6 |
| WV-8708 | 51.6 | 77.1 | 95.9 | 26.7 | 42.4 | 56.7 |
| WV-8709 | 33.3 | 60.8 | 24.3 | 16.5 | 30.6 | 11.9 |
| WV-8710 | 46.3 | 61.6 | 48.2 | 23.3 | 27.5 | 21.9 |
| WV-8711 | 62.6 | 68.0 | 56.3 | 30.6 | 28.9 | 27.7 |
| WV-8712 | 50.2 | 53.2 | 48.4 | 31.0 | 30.2 | 24.6 |
| WV-8713 | 32.5 | 41.1 | 34.2 | 20.6 | 22.4 | 21.3 |

ND33947 rs362307 SNP 10 uM Normalized

| | | | | | | |
|---|---|---|---|---|---|---|
| PBS | 83.5 | 93.1 | 123.4 | 95.9 | 90.4 | 131.3 |
| WV-993 | 24.0 | 90.9 | 67.5 | 27.2 | 94.7 | 70.4 |
| WV-1510 | 33.3 | 81.7 | 61.6 | 23.0 | 82.5 | 47.4 |
| WV-2603 | 64.6 | 87.3 | 78.1 | 41.7 | 85.6 | 65.7 |
| WV-4241 | 65.4 | 89.9 | 52.4 | 54.5 | 86.4 | 51.4 |
| WV-4242 | 114.6 | 103.6 | 54.4 | 123.2 | 109.1 | 57.9 |
| WV-4243 | 39.1 | 78.9 | 69.0 | 36.6 | 85.8 | 71.1 |
| WV-4244 | 28.2 | 46.8 | 59.3 | 31.7 | 52.6 | 62.4 |
| WV-8706 | 86.9 | 83.8 | 93.8 | 16.8 | 14.5 | 17.2 |
| WV-8707 | 53.9 | 64.8 | 64.8 | 8.0 | 12.4 | 7.6 |
| WV-8708 | 29.7 | 35.5 | 24.9 | 8.3 | 6.6 | 7.2 |
| WV-8709 | 22.0 | 21.7 | 19.7 | 6.3 | 5.9 | 6.8 |
| WV-8710 | 24.9 | 30.9 | 20.7 | 4.7 | 6.5 | 3.4 |
| WV-8711 | 28.6 | 27.9 | 23.4 | 6.1 | 9.6 | 7.0 |
| WV-8712 | 12.7 | 17.3 | 13.5 | 3.0 | 3.7 | 4.7 |
| WV-8713 | 17.9 | 18.3 | 11.1 | 5.2 | 6.5 | 5.0 |

GM01169 rs362331 SNP 2.5 uM Normalized

| | | | | | | |
|---|---|---|---|---|---|---|
| PBS | 98.9 | 123.5 | 100.4 | 92.0 | 113.6 | 94.4 |
| WV-993 | 69.8 | 89.6 | 82.6 | 67.7 | 87.8 | 78.5 |
| WV-1510 | 61.3 | 60.8 | 68.2 | 60.7 | 51.1 | 60.7 |
| WV-2603 | 71.5 | 78.8 | 83.1 | 64.4 | 68.6 | 70.7 |
| WV-4241 | 92.3 | 87.6 | 106.4 | 88.6 | 82.3 | 101.0 |
| WV-4242 | 50.4 | 99.7 | 100.9 | 48.9 | 94.7 | 94.8 |
| WV-4243 | 47.4 | 57.2 | 72.5 | 45.0 | 53.7 | 68.1 |
| WV-4244 | 28.7 | 62.4 | 62.6 | 25.6 | 59.9 | 55.0 |
| WV-8706 | 106.4 | 96.6 | 124.9 | 71.3 | 65.7 | 79.9 |
| WV-8707 | 87.6 | 123.5 | 106.7 | 50.8 | 77.8 | 59.7 |
| WV-8708 | 60.8 | 79.5 | 99.1 | 38.3 | 51.7 | 64.4 |
| WV-8709 | 59.6 | 91.0 | 87.9 | 38.2 | 56.4 | 55.4 |
| WV-8710 | 67.5 | 77.3 | 83.6 | 36.4 | 35.6 | 41.8 |
| WV-8711 | 85.0 | 91.4 | 95.6 | 47.6 | 46.6 | 50.7 |
| WV-8712 | 47.4 | 43.1 | 65.2 | 28.5 | 24.1 | 30.6 |
| WV-8713 | 47.5 | 47.9 | 46.7 | 32.3 | 32.5 | 30.4 |

GM01169 rs362331 SNP 10 uM Normalized

| | | | | | | |
|---|---|---|---|---|---|---|
| PBS | 90.9 | 100.0 | 131.6 | 90.0 | 85.0 | 125.1 |
| WV-993 | 25.5 | 92.6 | 70.6 | 26.1 | 94.5 | 68.4 |
| WV-1510 | 36.8 | 85.7 | 61.7 | 19.9 | 79.7 | 48.1 |
| WV-2603 | 67.8 | 88.9 | 81.9 | 39.3 | 85.3 | 63.0 |
| WV-4241 | 65.6 | 91.1 | 57.8 | 55.1 | 86.5 | 46.8 |
| WV-4242 | 124.2 | 104.6 | 56.0 | 115.5 | 109.8 | 57.2 |
| WV-4243 | 39.7 | 82.6 | 71.0 | 36.6 | 83.4 | 70.3 |
| WV-4244 | 30.6 | 51.3 | 64.2 | 29.7 | 48.8 | 58.4 |
| WV-8706 | 84.2 | 79.2 | 91.7 | 20.2 | 19.8 | 20.2 |
| WV-8707 | 52.4 | 65.3 | 61.8 | 10.0 | 12.4 | 11.1 |
| WV-8708 | 28.0 | 32.4 | 24.7 | 10.3 | 10.1 | 7.7 |
| WV-8709 | 17.8 | 19.8 | 18.2 | 10.7 | 8.1 | 8.5 |
| WV-8710 | 25.2 | 31.6 | 20.4 | 4.5 | 6.0 | 3.9 |
| WV-8711 | 28.4 | 30.2 | 24.5 | 6.5 | 7.6 | 6.1 |
| WV-8712 | 14.0 | 16.7 | 15.1 | 1.8 | 4.5 | 3.2 |
| WV-8713 | 18.0 | 19.2 | 12.8 | 5.4 | 5.8 | 3.4 |

Various HTT oligonucleotides were tested for stability.
Oligonucleotides were tested for stability in brain homogenate for 0, 2 or 5 days. Some day 5 time-points are eliminated due to sample contamination. 100 would represent the initial amount of oligonucleotide present (e.g., 100%), and 0.0 would represent no remaining oligonucleotide (0.0% remaining).

TABLE 13
Activity of certain oligonucleotides.

| Time (days) | 0 | 2 | 5 |
|---|---|---|---|
| WV-1497 | 100 | 40.6 | 15.6 |
| | 100 | 46.0 | 25.0 |
| | 100 | 53.2 | |
| WV-2688 | 100 | 44.7 | 16.0 |
| | 100 | 43.1 | 16.0 |
| | 100 | 56.2 | 22.4 |
| WV-8706 | 100 | 79.1 | |
| | 100 | 63.0 | |
| | 100 | 82.8 | 76.1 |
| WV-8707 | 100 | 89.5 | 79.7 |
| | 100 | 103.5 | 79.2 |
| | 100 | 85.1 | 78.6 |
| WV-8708 | 100 | 90.5 | |
| | 100 | 74.6 | 67.2 |
| | 100 | 91.8 | 80.3 |
| WV-8709 | 100 | 76.2 | 62.6 |
| | 100 | 77.7 | 63.1 |
| | 100 | 91.1 | 76.6 |
| WV-1092 | 100 | 17.6 | 6.4 |
| | 100 | 19.8 | 6.8 |
| | 100 | 19.7 | 11.4 |

HTT oligonucleotides which comprise the wild-type isoform of a SNP were constructed; these can act as surrogates for corresponding HTT oligonucleotides comprising a mutant isoform of a SNP. Surrogate HTT oligonucleotides were tested for their ability to knock down wild-type HTT in wild-type neurons (which does not comprise a mutant HTT allele), using gymnotic uptake.
Numbers indicate the % of HTT remaining (relative to control) at an oligonucleotide concentration of 10 uM, using gymnotic delivery. 100.0 would represent 100% HTT level (0% knockdown) and 0.0 would represent 000 HTT level (100% knock down).

TABLE 14
Activity of certain oligonucleotides.

| | | | |
|---|---|---|---|
| WV-9692 | 118.9 | 107.5 | 102.9 |
| WV-9693 | 99.9 | 107.9 | 110.3 |
| WV-9679 | 65.3 | 61.9 | 63.5 |
| WV-9660 | 92.6 | 86.3 | 94.4 |
| WV-9661 | 99.2 | 78.2 | 99.3 |
| WV-9662 | 84.6 | 89.7 | 92.2 |
| WV-9663 | 94.7 | 83.8 | 91.2 |
| WV-9664 | 109.2 | 109 | 103.3 |
| WV-9665 | 102.1 | 100.1 | 102.7 |
| WV-9666 | 106.6 | 99.6 | 90.1 |
| WV-9667 | 90.4 | 100.4 | 95.2 |
| WV-9668 | 93.5 | 93.3 | 89.6 |
| WV-9669 | 100 | 95.1 | 106.7 |
| WV-9491 (negative control) | 106.2 | 87.8 | 95.9 |
| Non-Treated | 88.9 | 95.8 | 110 |

Various HTT oligonucleotides, which are ssRNAi agents, targeting SNP rs362307 were constructed and tested for efficacy in vitro. In this Dual Luciferase assay, oligonucleotides were co-transfected into COS7 cells with plasmids expressing wild-type or mutant human HTT.
Concentrations of oligonucleotides used were: 3 nM, nM or 0.33 nM.
H₂O was used as a negative control.
Numbers indicate the % of HTT remaining (relative to control) at the indicated oligonucleotide concentrations. 1.00 would represent 100% HTT mRNA remaining (0.0% knockdown); and 0.0 will represent 0.00% HTT mRNA remaining (100.0% knockdown).

TABLE 15
Activity of certain oligonucleotides.

| | | |
|---|---|---|
| 3 nM | 1 nM | 0.33 nM |

Knockdown of Wild-type HTT

| | | | | | | |
|---|---|---|---|---|---|---|
| H₂O | 1.10 | 1.04 | 1.00 | 0.96 | 1.01 | 1.04 |
| WV-1497 | 0.90 | 0.92 | 0.96 | 0.99 | 1.10 | 0.96 |
| WV-2477 | 1.01 | 0.99 | 1.05 | 0.95 | 0.96 | 1.04 |
| WV-10107 | 0.55 | 0.53 | 0.66 | 0.62 | 0.84 | 0.85 |
| WV-10108 | 0.66 | 0.62 | 0.67 | 0.69 | 0.94 | 0.91 |
| WV-10109 | 0.75 | 0.73 | 0.72 | 0.76 | 0.86 | 0.94 |
| WV-10110 | 0.82 | 0.78 | 0.91 | 0.83 | 0.95 | 1.01 |
| WV-10111 | 0.92 | 0.76 | 0.84 | 0.80 | 1.01 | 0.98 |
| WV-10112 | 0.72 | 0.78 | 0.79 | 0.87 | 0.94 | 1.13 |
| WV-10113 | 0.56 | 0.56 | 0.61 | 0.66 | 0.80 | 0.98 |
| WV-10114 | 0.69 | 0.67 | 0.73 | 0.76 | 0.95 | 1.15 |
| WV-10115 | 0.32 | 0.39 | 0.45 | 0.48 | 0.68 | 0.80 |
| WV-10116 | 0.52 | 0.58 | 0.68 | 0.68 | 0.86 | 0.91 |
| WV-10117 | 0.64 | 0.71 | 0.77 | 0.79 | 0.96 | 1.15 |
| WV-10118 | 0.79 | 0.76 | 0.75 | 0.79 | 1.00 | 1.15 |
| WV-10119 | 0.88 | 0.88 | 0.86 | 0.93 | 1.05 | 1.12 |

Knockdown of mutant HTT

| | | | | | | |
|---|---|---|---|---|---|---|
| H₂O | 1.16 | 1.07 | 1.09 | 1.07 | 1.13 | 1.02 |
| WV-1497 | 0.64 | 0.58 | 0.92 | 0.94 | 0.99 | 0.97 |
| WV-2477 | 1.02 | 0.98 | 1.00 | 1.00 | 1.00 | 1.00 |
| WV-10107 | 0.42 | 0.41 | 0.59 | 0.55 | 0.79 | 0.78 |
| WV-10108 | 0.52 | 0.52 | 0.69 | 0.69 | 0.86 | 0.88 |
| WV-10109 | 0.53 | 0.61 | 0.66 | 0.72 | 0.84 | 0.90 |
| WV-10110 | 0.78 | 0.77 | 0.87 | 0.83 | 0.91 | 0.89 |
| WV-10111 | 0.77 | 0.75 | 0.95 | 1.00 | 1.06 | 0.98 |
| WV-10112 | 0.73 | 0.74 | 0.92 | 0.84 | 0.96 | 0.96 |
| WV-10113 | 0.53 | 0.55 | 0.71 | 0.74 | 0.84 | 0.86 |
| WV-10114 | 0.67 | 0.64 | 0.85 | 0.84 | 0.86 | 0.95 |
| WV-10115 | 0.31 | 0.33 | 0.49 | 0.43 | 0.68 | 0.73 |
| WV-10116 | 0.55 | 0.59 | 0.77 | 0.76 | 0.93 | 0.98 |
| WV-10117 | 0.67 | 0.63 | 0.80 | 0.79 | 1.00 | 1.02 |
| WV-10118 | 0.72 | 0.67 | 0.91 | 0.87 | 1.02 | 1.09 |
| WV-10119 | 0.84 | 0.86 | 0.96 | 0.95 | 1.05 | 1.08 |

Knockdown of Wild-type HTT

| | | | | | | |
|---|---|---|---|---|---|---|
| H₂O | 0.98 | 0.97 | 1.01 | 1.06 | 1.02 | 0.97 |
| WV-1497 | 0.84 | 0.82 | 1.04 | 0.93 | 1.03 | 0.96 |
| WV-2477 | 1.07 | 0.93 | 1.03 | 0.97 | 1.00 | 1.00 |
| WV-10120 | 0.68 | 0.70 | 0.85 | 0.90 | 0.96 | 0.68 |
| WV-10121 | 0.71 | 0.74 | 0.91 | 0.83 | 0.87 | 0.94 |
| WV-10122 | 0.67 | 0.61 | 0.82 | 0.91 | 0.96 | 0.90 |
| WV-10123 | 0.60 | 0.64 | 0.79 | 0.84 | 0.88 | 0.90 |
| WV-10124 | 0.69 | 0.74 | 0.83 | 0.88 | 1.04 | 1.04 |
| WV-10125 | 0.84 | 0.85 | 0.97 | 0.98 | 1.01 | 1.14 |
| WV-10126 | 0.78 | 0.80 | 1.01 | 0.99 | 0.96 | 0.95 |
| WV-10127 | 0.46 | 0.41 | 0.65 | 0.62 | 0.81 | 0.92 |
| WV-10128 | 0.75 | 0.76 | 1.03 | 0.97 | 1.10 | 1.08 |
| WV-10129 | 0.81 | 0.86 | 0.95 | 1.03 | 0.96 | 1.09 |
| WV-10130 | 0.85 | 0.87 | 1.03 | 1.01 | 1.04 | 1.15 |
| WV-10131 | 0.74 | 0.77 | 0.96 | 1.00 | 1.10 | 1.19 |
| WV-10132 | 0.65 | 0.64 | 0.92 | 0.86 | 1.08 | 1.03 |

Knockdown of mutant HTT

| | | | | | | |
|---|---|---|---|---|---|---|
| H₂O | 1.20 | 0.96 | 0.99 | 0.93 | 1.03 | 0.97 |
| WV-1497 | 0.66 | 0.60 | 0.88 | 0.85 | 0.95 | 0.97 |
| WV-2477 | 0.97 | 1.03 | 1.04 | 0.96 | 1.01 | 0.99 |
| WV-10120 | 0.81 | 0.71 | 0.86 | 0.77 | 0.93 | 0.99 |
| WV-10121 | 0.91 | 0.80 | 0.92 | 0.87 | 0.96 | 0.96 |
| WV-10122 | 0.76 | 0.74 | 0.78 | 0.79 | 0.94 | 0.88 |
| WV-10123 | 0.69 | 0.64 | 0.81 | 0.79 | 0.92 | 0.86 |
| WV-10124 | 0.70 | 0.72 | 0.79 | 0.81 | 0.88 | 0.88 |
| WV-10125 | 0.82 | 0.85 | 0.95 | 0.90 | 0.91 | 1.09 |
| WV-10126 | 0.93 | 0.95 | 1.00 | 1.00 | 0.96 | 1.15 |
| WV-10127 | 0.39 | 0.39 | 0.50 | 0.46 | 0.71 | 0.79 |
| WV-10128 | 0.67 | 0.63 | 0.74 | 0.79 | 0.95 | 1.15 |
| WV-10129 | 0.85 | 0.86 | 0.91 | 0.87 | 0.89 | 1.07 |
| WV-10130 | 0.73 | 0.77 | 0.78 | 0.84 | 0.99 | 1.07 |
| WV-10131 | 0.73 | 0.68 | 0.81 | 0.85 | 0.91 | 1.03 |
| WV-10132 | 0.53 | 0.61 | 0.66 | 0.82 | 0.95 | 1.08 |

Knockdown of Wild-type HTT

| | | | | | | |
|---|---|---|---|---|---|---|
| WV-1497 | 0.61 | 0.60 | 0.64 | 0.60 | 0.75 | 0.76 |
| WV-2477 | 0.99 | 1.01 | 1.01 | 0.99 | 1.00 | 1.00 |
| WV-10133 | 1.13 | 1.07 | 0.98 | 0.97 | 0.95 | 0.99 |
| WV-10134 | 0.88 | 0.85 | 0.87 | 0.89 | 1.02 | 0.93 |
| WV-10135 | 0.54 | 0.58 | 0.66 | 0.68 | 0.79 | 0.77 |
| WV-10136 | 0.74 | 0.76 | 0.79 | 0.77 | 0.93 | 0.93 |
| WV-10137 | 0.77 | 0.68 | 0.74 | 0.71 | 0.81 | 0.84 |
| WV-10138 | 1.04 | 1.03 | 0.94 | 0.90 | 0.99 | 0.99 |
| WV-10139 | 1.01 | 1.02 | 0.80 | 0.95 | 0.93 | 0.96 |
| WV-10140 | 0.90 | 0.91 | 0.85 | 0.88 | 1.00 | 1.03 |
| WV-10141 | 0.97 | 1.01 | 0.88 | 0.88 | 1.02 | 0.99 |
| WV-10142 | 0.86 | 0.83 | 0.80 | 0.82 | 0.88 | 1.04 |
| WV-10143 | 0.78 | 0.73 | 0.79 | 0.70 | 0.89 | 1.15 |
| WV-10144 | 0.68 | 0.70 | 0.73 | 0.63 | 0.90 | 0.99 |
| WV-10145 | 1.12 | 0.97 | 0.92 | 0.90 | 0.93 | 0.99 |
| WV-10146 | 1.20 | 1.14 | 1.00 | 1.10 | 1.10 | 1.14 |

Knockdown of mutant HTT

| | | | | | | |
|---|---|---|---|---|---|---|
| WV-1497 | 0.99 | 0.97 | 0.79 | 0.77 | 0.92 | 0.93 |
| WV-2477 | 1.08 | 0.92 | 0.96 | 1.04 | 0.97 | 1.03 |
| WV-10133 | 1.87 | 1.65 | 1.26 | 1.36 | 1.20 | 1.18 |
| WV-10134 | 1.09 | 1.05 | 0.92 | 0.91 | 0.92 | 0.95 |
| WV-10135 | 0.70 | 0.65 | 0.56 | 0.65 | 0.79 | 0.76 |
| WV-10136 | 0.99 | 0.84 | 0.72 | 0.80 | 0.93 | 0.88 |
| WV-10137 | 0.83 | 0.87 | 0.63 | 0.68 | 0.79 | 0.73 |
| WV-10138 | 1.18 | 1.20 | 0.92 | 1.01 | 1.02 | 0.97 |
| WV-10139 | 1.66 | 1.44 | 1.16 | 1.18 | 1.16 | 1.22 |
| WV-10140 | 1.51 | 1.40 | 1.12 | 1.08 | 1.08 | 1.13 |
| WV-10141 | 1.68 | 1.57 | 1.46 | 1.13 | 1.19 | 1.17 |
| WV-10142 | 1.21 | 1.19 | 0.95 | 0.90 | 1.03 | 0.94 |
| WV-10143 | 1.20 | 1.06 | 0.99 | 0.96 | 1.19 | 1.08 |
| WV-10144 | 1.15 | 1.09 | 0.83 | 0.80 | 0.93 | 0.98 |
| WV-10145 | 1.69 | 1.51 | 1.23 | 1.17 | 1.13 | 1.10 |
| WV-10146 | 1.74 | 1.65 | 1.21 | 1.12 | 1.13 | 1.13 |

Various HTT oligonucleotides comprising different patterns of stereochemistry and/or different 2′-modifications (or patterns thereof) were tested in vitro for their ability to knock down wild-type (wt) and the mutant (in) HTT corresponding to the SNP.
Results are shown below. Cells were treated with oligonucleotides at concentrations of 3 nM or 30 nM.
Additional data was generated related to various other HTT oligonucleotides disclosed herein.
Potency of various HTT oligonucleotides targeting SNP rs362307 was determined in vitro, as measured by IC₅₀. The percent reduction of mu HTT mRNA is also provided. 0.0% would represent 100.0% HTT remaining (0.0% knockdown) and 100.0 would represent 0.0% HTT remaining (100.0% knockdown). Data are from replicates and average are shown. This and the next table represent composite data derived from multiple experiments.

| | WV-12544 | WV-11972 | WV-13628 |
|---|---|---|---|
| % total knockdown | 48% | 61% | 71% |
| at 10 μM | | | |
| IC₅₀ | 9.8 μM | 8 μM | 3.3 μM |

The potency of various HTT oligonucleotides to rs362273 was also tested in vitro.

| | WV-9679 | WV-12282 | WV-12283 | WV-12284 |
|---|---|---|---|---|
| % total knockdown | 75% | 51% | 0% | 49% |
| at 10 μM | | | | |
| IC₅₀ | 3.2 μM | 3848 nM | >10 uM | 4016 nM |
| Selectivity: % WT HTT | | 8% at 20 nM | 0% at 20 nM | |
| reduction | | | | |
| Selectivity: % MU HTT | | 82% at 20 nM | 48% at 20 nM | |
| reduction | | | | |

% total knockdown at 10 μM indicates amount of reduction of total HTT in human iPSC-derived neurons, wherein both alleles of HTT are wild-type.
IC₅₀ was also determined in human iPSC-derived neurons.
Selectivity was tested in vitro in the reporter assay described herein.

### Table 16. Activity of Certain Oligonucleotides.

An experiment was performed to test the activity of various HTT oligonucleotides in BacHD mice 1 wk and 2 week (wk) post 1×100 g administration ICV.
A goal was to confirm knockdown and explore a time course of human HTT transcripts with various HTT oligonucleotides after Single ICV injections in BACHD mice. Several HTT oligonucleotides were chosen based on their robust activity in in vitro assays (iCell neurons); WV-9679 was used as positive control. The tested HTT oligonucleotides had different patterns of stereochemistry, and some comprise one or more non-negatively charged internucleotidic linkage. Knockdown of HTT was tested in hippocampus, cortex and striatum.
Animals used: BACHD mice, 8-12 week-old, 6 groups, 36 mice; Method: ICV cannulation; ICV injections of PBS or HTT oligonucleotide on Day 1 in awake animals; Necropsy 1 and 2 weeks after dosing. For Necropsy: whole body perfusion with PBS; Flush out spinal cord (PK and PD analysis); dissect one hemibrain (cortex, hippocampus, striatum) into 2 ml Eppendorf tubees, flash freeze (PK and PD analysis); and Second hemibrain also dissected and flash frozen for PK and PD.

### Groups of Animals:

| | | | Dosing | Dose | |
|---|---|---|---|---|---|
| Group | Test Article | Dose | Regimen | Volume | # mice |
| 1 | PBS | NA | ICV, day 1 | 2.5 ml | 6 |
| 2 | WV-9679 | 1 × 100 mg | ICV, day 1 | 2.5 ml | 6 |
| 3 | WV-15080 | 1 × 100 mg | ICV, day 1 | 2.5 ml | 6 |
| 4 | WV-14914 | 1 × 100 mg | ICV, day 1 | 2.5 ml | 6 |
| 5 | WV-12282 | 1 × 100 mg | ICV, day 1 | 2.5 ml | 6 |
| 6 | WV-12284 | 1 × 100 mg | ICV, day 1 | 2.5 ml | 6 |

All animals were BacHD mice, age 8-12 weeks (wks). Necropsy was performed on Day 8 and 15 for all groups.
Results are shown below.

TABLE 17
Activity of certain oligonucleotides.
Numbers indicate hHTT (human HTT or hHD)/TUBB3, relative to PBS. 1.0
would represent 100% HTT level (0% knockdown) and 0.0 would represent
0% HTT level (100% knock down).

| | | | | | |
|---|---|---|---|---|---|
| PBS | WV-9679 | WV-15080 | WV-14914 | WV-12282 | WV-12284 |

Cortex, 2 × 50 μg.

| | | | | | |
|---|---|---|---|---|---|
| 0.763 | | 0.638 | 0.925 | 0.705 | 1.124 |
| 1.199 | 0.314 | 0.589 | 0.493 | 1.179 | 0.775 |
| 1.174 | 0.776 | 0.629 | | 1.083 | |
| 0.856 | 0.481 | 0.496 | 0.665 | 1.139 | 0.863 |
| | 0.919 | 0.647 | 0.802 | 1.113 | 0.769 |
| | | 0.415 | 0.891 | 1.043 | 1.155 |

Hippocampus, 2 × 50 μg.

| | | | | | |
|---|---|---|---|---|---|
| 1.069 | | 0.759 | 1.059 | 0.657 | 2.129 |
| 1.095 | 0.613 | 1.649 | 0.594 | 0.753 | 2 |
| 1.108 | 0.743 | 0.958 | | 0.996 | |
| 0.732 | 0.643 | 1.018 | 0.638 | 0.886 | 1.327 |
| | 1.094 | 0.689 | 0.733 | 1.106 | 1.394 |
| | | 0.362 | 0.911 | 1.014 | 1.184 |

Striatum, 2 × 50 μg.

| | | | | | |
|---|---|---|---|---|---|
| 0.905 | | 0.788 | 1.072 | 0.609 | 1.156 |
| 1.093 | 0.437 | 0.794 | 0.56 | 1.244 | 0.945 |
| 1.227 | 1.087 | 0.705 | | 0.965 | |
| 0.786 | 0.674 | 0.905 | 0.71 | 1.261 | 0.739 |
| | 0.755 | 0.594 | 1.005 | 1.028 | 0.91 |
| | | 0.52 | 1.049 | 1.171 | 1.082 |

Various oligonucleotides to any of several HTT SNPs were tested for knockdown of HTT in iNeurons from patient 100 or patient 1279 [also designated Pt100 (or Pt 100) or Pt01279 (or Pt 1279), respectively]. Oligonucleotides were delivered gymnotically at 10 uM, and cells were tested at Day 7. Numbers represent % of HTT remaining, wherein 100.0 would represent 100.0% HTT remaining (0.0% knockdown) and 0.00% would represent 0.00% HTT remaining (100.0% knockdown). Percentage of tubulin (TUBB Average) was also determined, where tubulin is a housekeeping gene for neural cells, and a significant decrease in tubulin might suggest, among several possibilities, toxicity mediated by an oligonucleotide. If two cell types were used, the TUBB Average represents the average across cell types. Replicates were performed, and in various cases numbers represent results of individual replicates or average of replicates. HTT/Tubulin ratios can be calculated from data presented herein. In various experiments (including data not shown) with HTT oligonucleotides and negative control oligonucleotides were used, including: WV-975, WV-975, WV-993, WV-993, WV-1061, WV-1061, WV-1062, WV-1062, WV-1063, WV-1063, WV-1064, WV-1064, WV-1065, WV-1065, WV-1066, WV-1066, each of which is also described in WO2017/192664.
Various oligonucleotides to HTT SNP rs362331 were tested for knockdown of WT HTT in iNeurons from patient 100 or patient 1279, which are both homozygous for WT HTT at this SNP. WV-993, which does not target HTT, was used as a negative control. Oligonucleotides were delivered at 10 uM, and cells were tested at Day 7. Numbers represent % of HTT remaining, wherein 100.0 would represent 100.0% HTT remaining (0.0% knockdown) and 0.0% would represent 0.0% HTT remaining (100.0% knockdown). Percentage oftubulin (TUBB average) was also determined, wherein 100.0 would represent 100.0% tubulin remaining and 0.0% would represent 0.0% tubulin remaining. The ratio of HTT/tubulin can be calculated from the presented data.

TABLE 18
Activity of certain oligonucleotides.

| | % HTT | % HTT | % HTT | |
|---|---|---|---|---|
| | remaining | remaining | remaining | TUBB |
| Oligonucleotide | (Pt 100) | (Pt 1279) | (Pt 1279) | Average |
| WV-993 | 89.00 | | | 101.0 |
| WV-8710 | 121 | 103.4 | 102.5 | 81.9 |
| WV-8711 | 80 | 107.2 | 108.1 | 100.6 |
| WV-8712 | 98.00 | 97.00 | 97.10 | 97.2 |
| WV-8713 | 112 | 100.4 | 85.9 | 83.7 |

Various oligonucleotides to HTT SNP rs362307 were tested for knockdown of WT HTT in iNeurons from patient 100 or patient 1279, which were homozygous for WT HTT at this SNP. WV-993 was the negative control. Oligonucleotides were delivered at 10 uM, and cells were tested at Day 7. Numbers represent % of HTT remaining, wherein 100.0 would represent 100.0% HTT remaining (0.0% knockdown) and 0.0% would represent 0.0% HTT remaining (100.0% knockdown). Percentage of tubulin (TUBB average) was also determined, wherein 100.0 would represent 100.0% tubulin remaining and 0.0% would represent 0.0% tubulin remaining. The ratio of HTT/tubulin is also shown. WV-9679 is a positive control.

TABLE 19
Activity of certain oligonucleotides.

| | % HTT | % HTT | % HTT | % HTT | |
|---|---|---|---|---|---|
| | remaining | remaining | remaining | remaining | TUBB |
| Oligonucleotide | (Pt 100) | (Pt 100) | (Pt 1279) | (Pt 1279) | Average |
| WV-9660 | 84.00 | 86.6 | | 85.30 | 111.2 |
| WV-9661 | 78.00 | 86.8 | | 84.10 | 114.2 |
| WV-9662 | 75.00 | 86.7 | | 82.10 | 122.3 |
| WV-9663 | 99.00 | 89.9 | | 142.60 | 94.8 |
| WV-9664 | 88.00 | 92.6 | | 78.60 | 115.3 |
| WV-9665 | 88.00 | 74.9 | | 69.80 | 123.8 |
| WV-9666 | 80.00 | 73.8 | 75.00 | 76.10 | 110.9 |
| WV-9667 | 88.00 | 77.5 | | 79.60 | 103.6 |
| WV-9668 | 79.00 | | | 83.50 | 105.5 |
| WV-9669 | 92.00 | | | 85.00 | 102.1 |
| WV-9679 | 31.00 | 26.00 | 26.00 | 24.00 | 106.0 |
| WV-9692 | | 93.8 | | | |
| WV-9693 | | 90.2 | | | |
| WV-10767 | 117 | | 101.4 | | 90.8 |
| WV-10768 | 113 | | 101.4 | | 87.2 |
| WV-10769 | 103 | | 88.2 | 72.667 | 111.4 |
| WV-10770 | 112 | | 109 | | 97.2 |
| WV-10771 | 116 | | 81.7 | | 103.2 |
| WV-10772 | 101 | | 84.1 | 88 | 97.9 |
| WV-10773 | 90 | | 70.2 | 84.333 | 83.8 |
| WV-10774 | 95 | | 88.4 | 93.333 | 111.6 |
| WV-10775 | 106 | | 89.7 | 102.67 | 99.9 |
| WV-10776 | 103 | | 92.6 | 75 | 106.7 |

Various HTT oligonucleotides which target an intronic site were tested for knockdown of WT HTT in iNeurons from Pt 100. Oligonucleotides were delivered at 10 uM, and cells were tested at Day 7. Numbers represent % of HTT remaining, wherein 100.0 would represent 100.0% HTT remaining (0.0% knockdown) and 0.0% would represent 0.0% HTT remaining (100.0% knockdown).

TABLE 20
Activity of certain oligonucleotides.

| Oligonucleotide | % HTT remaining | TUBB Average |
|---|---|---|
| WV-10783 | 82.10 | 81.0 |
| WV-10784 | 87.17 | 86.0 |
| WV-10785 | 77.53 | 87.0 |
| WV-10786 | 53.38 | 87.0 |
| WV-10787 | 36.87 | 87.0 |
| WV-10788 | 56.58 | 82.0 |
| WV-10789 | 63.06 | 83.0 |
| WV-10790 | 30.97 | 145.0 |
| WV-10791 | 36.41 | 87.0 |
| WV-10792 | 76.81 | 84.0 |
| WV-10793 | 79.19 | 84.0 |
| WV-10794 | 73.39 | 82.0 |
| WV-10795 | 62.56 | 75.0 |
| WV-10796 | 81.88 | 80.0 |
| WV-10797 | 87.05 | 78.0 |
| WV-10798 | 102.25 | 183.0 |
| WV-10799 | 88.71 | 72.0 |
| WV-10800 | 73.03 | 73.0 |
| WV-10801 | 77.69 | 72.0 |
| WV-10802 | 86.23 | 72.0 |
| WV-10803 | 90.21 | 68.0 |
| WV-10804 | 81.70 | 68.0 |
| WV-10805 | 82.73 | 68.0 |
| WV-10806 | 47.05 | 153.0 |
| WV-10807 | 94.38 | 59.0 |
| WV-10808 | 72.58 | 61.0 |
| WV-10809 | 88.83 | 58.0 |
| WV-10810 | 52.76 | 57.0 |
| WV-10811 | 45.59 | 62.0 |
| WV-10812 | 78.79 | 62.0 |
| WV-10813 | 80.48 | 55.0 |
| WV-10814 | 107.64 | 67.0 |
| WV-10815 | 58.19 | 84.0 |
| WV-10816 | 73.74 | 96.0 |
| WV-10817 | 97.23 | 86.0 |

Various oligonucleotides to HTT SNP rs362099 were tested for knockdown of HTT in iNeurons from patient 100, which were heterozygous mu/WT HTT at this SNP. Oligonucleotides were delivered at 10 uM, and cells were tested at Day 7. Numbers represent % of HTT remaining, wherein 100.0 would represent 100.0% HTT remaining (0.0% knockdown) and 0.0% would represent 0.0% HTT remaining (100.0% knockdown). Percentage of tubulin (TUBB average) was also determined, wherein 100.0 would represent 100.0% tubulin remaining and 0.0% would represent 0.0% tubulin remaining.

TABLE 21
Activity of certain oligonucleotides.

| | | | | | | TUBB |
|---|---|---|---|---|---|---|
| Oligonucleotide | % HTT remaining | | | | | average |
| WV-10889 | 96.59 | 103.68 | | | | 92.0 |
| WV-10890 | 94.85 | 84.14 | | | | 92.5 |
| WV-10891 | 79.60 | 78.04 | | | | 91.0 |
| WV-10892 | 83.30 | 115.62 | | | | 79.5 |
| WV-10893 | 83.27 | 90.19 | | | | 95.5 |
| WV-10894 | 98.27 | 81.01 | | | | 91.0 |
| WV-10895 | 86.38 | 85.12 | | | | 93.5 |
| WV-10896 | 103.00 | 108.97 | | | | 106.0 |
| WV-10897 | 102.60 | | | | | 93.0 |
| WV-10898 | 100.80 | | | | | 120.0 |
| WV-10899 | 100.80 | | | | | 97.0 |
| WV-10900 | 112.50 | | | 87.00 | | 95.0 |
| WV-10901 | 106.60 | | | 86.67 | | 98.0 |
| WV-10902 | 106.90 | | | 94.67 | | 92.0 |
| WV-10903 | 112.70 | | | 87.00 | | 86.0 |
| WV-10904 | | | | 77.50 | | |
| WV-10905 | | 106.37 | | 85.50 | 79.591 | 67.0 |
| WV-10906 | | 106.33 | | 76.00 | 89.22 | 71.0 |
| WV-10907 | | 103.23 | | 90.00 | 89.914 | 71.0 |
| WV-10908 | | 97.70 | | 86.33 | 85.346 | 72.0 |
| WV-10909 | | 95.65 | 38.00 | | 60.565 | 71.0 |
| WV-10910 | | 107.11 | 58.67 | | 65.181 | 62.0 |
| WV-10911 | | 87.51 | 62.67 | | 61.746 | 71.0 |
| WV-10912 | | 104.08 | 50.33 | | 55.179 | 69.0 |
| WV-10913 | | 89.75 | 67.00 | | 52.708 | 99.0 |
| WV-10914 | | 102.61 | 65.00 | | 73.359 | 90.0 |
| WV-10915 | | 102.22 | 68.33 | | 85.568 | 90.0 |
| WV-10916 | | | 77.67 | | 79.987 | |
| WV-10917 | | | 53.00 | | 65.398 | |
| WV-10918 | | | 52.67 | | 63.903 | |
| WV-10919 | | | 63.00 | | 41.534 | |
| WV-10920 | | | 71.00 | | 57.478 | |
| WV-10921 | | | 77.00 | | | |
| WV-10922 | | | 82.33 | | | |
| WV-10923 | | | 66.00 | | | |
| WV-10924 | | | 78.33 | | | |
| WV-10925 | | | 81.67 | | | |
| WV-10926 | | | 76.33 | | | |
| WV-10927 | | | 69.67 | | | |
| WV-10928 | | | 64.00 | | | |
| WV-10929 | | | 72.33 | | | |
| WV-10930 | | | 72.67 | | | |
| WV-10931 | | | 68.00 | | | |
| WV-10932 | | | 71.00 | | | |
| WV-10933 | | | 63.33 | | | |
| WV-10934 | | | 79.67 | | | |
| WV-10935 | | | 80.00 | | | |
| WV-10936 | | | 83.33 | | | |
| WV-10937 | | | 76.33 | | | |
| WV-10938 | | | 71.33 | | | |

Various oligonucleotides to HTT SNP rs262273 were tested for knockdown of HTT in iNeurons from patient 100, which were heterozygous mu/WT HTT at this SNP. Oligonucleotides were delivered at 10 uM, and cells were tested at Day 7. Numbers represent % of HTT remaining, wherein 100.0 would represent 100.000 HTT remaining (0.0% knockdown) and 0.0% would represent 0.0% HTT remaining (100.0% knockdown). Percentage of tubulin (TUBB average) was also determined, wherein 100.0 would represent 100.0% tubulin remaining and 0.0% would represent 0.0% tubulin remaining.

TABLE 22
Activity of certain oligonucleotides.

| | | |
|---|---|---|
| Oligonucleotide | % HTT remaining | TUBB Average |

| | | | |
|---|---|---|---|
| WV-10939 | | 79 | |
| WV-10940 | | 85.667 | |
| WV-10941 | | 87.667 | |
| WV-10942 | | 85.667 | |
| WV-10943 | | 83 | |
| WV-10944 | | 87.667 | |
| WV-10945 | | 103.33 | |
| WV-10946 | | 85.667 | |
| WV-10947 | | 89 | |
| WV-10948 | | 100 | |
| WV-10949 | | 87.33 | |
| WV-10950 | | 89.33 | |
| WV-10951 | | 85.67 | |
| WV-10952 | | 87.00 | |
| WV-10953 | | 98.33 | |
| WV-10954 | | 84.00 | |
| WV-10955 | | 89.67 | |
| WV-10956 | | 91.33 | |
| WV-10957 | | 95.33 | |
| WV-10958 | | 99.00 | |
| WV-10959 | | 85.67 | |
| WV-10960 | | 86.67 | |
| WV-10961 | 89.45 | 94.67 | 101.0 |
| WV-10962 | 75.34 | 84.33 | 112.0 |
| WV-10963 | 75.24 | 74.00 | 99.0 |
| WV-10964 | 68.61 | 93.33 | 113.0 |
| WV-10965 | 88.78 | 88.00 | 107.0 |
| WV-10966 | 85.53 | 72.67 | 99.0 |
| WV-10967 | 86.10 | 75.00 | 97.0 |
| WV-10968 | 100.20 | 102.67 | 130.0 |
| WV-10969 | 84.94 | 85.67 | 111.0 |
| WV-10970 | 74.07 | 86.67 | 115.0 |
| WV-10971 | 102.20 | 94.67 | 105.0 |
| WV-10972 | 92.82 | 84.33 | 104.0 |
| WV-10973 | 95.42 | 98.00 | 106.0 |
| WV-10974 | 85.78 | 86.33 | 111.0 |
| WV-10975 | 92.04 | 92.33 | 107.0 |
| WV-10976 | 95.37 | 90.00 | 149.0 |
| WV-10977 | 76.34 | 90.33 | 111.0 |
| WV-10978 | 80.07 | 83.00 | 112.0 |
| WV-10979 | 81.21 | | 113.0 |
| WV-10980 | 82.57 | | 108.0 |
| WV-10981 | 89.97 | | 110.0 |
| WV-10982 | 86.80 | | 108.0 |
| WV-10983 | 83.88 | | 111.0 |
| WV-10984 | 88.13 | | 152.0 |
| WV-10985 | 92.10 | | 103.0 |
| WV-10986 | 90.18 | | 101.0 |
| WV-10987 | 85.91 | | 99.0 |
| WV-10988 | 93.68 | | 97.0 |

Various oligonucleotides to HTT SNP rs362272 were tested for knockdown of HTT in iNeurons from patient 100, which were heterozygous mu/WT HTT at this SNP. Oligonucleotides were delivered at 10 uM, and cells were tested at Day 7. Numbers represent % of HTT remaining, wherein 100.0 would represent 100.0% HTT remaining (0.0% knockdown) and 0.0% would represent 0.0% HTT remaining (100.0% knockdown). Percentage of tubulin (TUBB average) was also determined, wherein 100.0 would represent 100.0% tubulin remaining and 0.0% would represent 0.0% tubulin remaining.

TABLE 23
Activity of certain oligonucleotides.

| Oligonucleotide | % HTT remaining | TUBB Average |
|---|---|---|
| WV-10989 | 92.61 | 94.0 |
| WV-10990 | 92.96 | 100.0 |
| WV-10991 | 99.68 | 95.0 |
| WV-10992 | 106.20 | 133.0 |
| WV-10993 | 88.87 | 104.0 |
| WV-10994 | 92.11 | 106.0 |
| WV-10995 | 91.35 | 114.0 |
| WV-10996 | 92.87 | 106.0 |
| WV-10997 | 91.57 | 109.0 |
| WV-10998 | 100.90 | 100.0 |
| WV-10999 | 101.60 | 97.0 |
| WV-11000 | 105.70 | 151.0 |
| WV-11001 | 85.16 | 111.0 |
| WV-11002 | 82.54 | 110.0 |
| WV-11003 | 95.20 | 99.0 |
| WV-11004 | 123.70 | 81.0 |
| WV-11005 | 98.35 | 100.0 |
| WV-11006 | 97.02 | 99.0 |
| WV-11007 | 97.35 | 101.0 |
| WV-11008 | 109.10 | 148.0 |
| WV-11009 | 90.89 | 109.0 |
| WV-11010 | 81.30 | 110.0 |
| WV-11011 | 87.92 | 101.0 |
| WV-11012 | 84.15 | 104.0 |
| WV-11013 | 95.77 | 100.0 |
| WV-11014 | 90.80 | 103.0 |
| WV-11015 | 94.09 | 100.0 |
| WV-11016 | 111.30 | 139.0 |
| WV-11017 | 98.77 | 103.0 |
| WV-11018 | 85.58 | 110.0 |
| WV-11019 | 95.30 | 106.0 |
| WV-11020 | 93.13 | 79.0 |
| WV-11021 | 92.19 | 103.0 |
| WV-11022 | 94.71 | 99.0 |
| WV-11023 | 94.61 | 95.0 |
| WV-11024 | 102.00 | 136.0 |
| WV-11025 | 104.80 | 99.0 |
| WV-11026 | 100.80 | 106.0 |
| WV-11027 | 104.00 | 97.0 |
| WV-11028 | 96.19 | 99.0 |
| WV-11029 | 96.26 | 103.0 |
| WV-11030 | 92.74 | 96.0 |
| WV-11031 | 99.19 | 98.0 |
| WV-11032 | 107.70 | 125.0 |
| WV-11033 | 103.60 | 103.0 |
| WV-11034 | 101.20 | 103.0 |
| WV-11035 | 104.40 | 100.0 |
| WV-11036 | 101.00 | 91.0 |
| WV-11037 | 99.55 | 100.0 |
| WV-11038 | 96.60 | 93.0 |

Various oligonucleotides to HTT SNP rs362307 were tested for knockdown of HTT in iNeurons from patient 1279, which were homozygous WT HTT at this SNP. Oligonucleotides were delivered at 10 uM, and cells were tested at Day 7. Numbers represent % of HTT remaining, wherein 100.0 would represent 100.00% HTT remaining (0.0% knockdown) and 0.00% would represent 0.00% HTT remaining (100.0% knockdown). Percentage oftubulin (TUBB average) was also determined, wherein 100.0 would represent 100.00% tubulin remaining and 0.00% would represent 0.00% tubulin remaining.

TABLE 24
Activity of certain oligonucleotides.

| | | |
|---|---|---|
| Oligonucleotide | % HTT remaining | TUBB Average |

| | | | |
|---|---|---|---|
| WV-11534 | 85.7 | 109.19 | 114.6 |
| WV-11535 | 89.6 | 86.905 | 105.3 |
| WV-11536 | 80.9 | 70.49 | 115.2 |
| WV-11537 | 77.2 | 74.396 | 120.9 |
| WV-11538 | 75.1 | 75.667 | 121.5 |
| WV-11539 | 80.6 | 77.643 | 132.2 |
| WV-11540 | 74.3 | 74.064 | 135.6 |
| WV-11541 | 65.3 | 80.982 | 114.4 |
| WV-11542 | 65.1 | 96.223 | 114.5 |
| WV-11543 | 70.8 | 96.887 | 116.2 |

Various oligonucleotides to HTT SNP rs362331 were tested for knockdown of HTT in iNeurons from patient 1279, which were homozygous WT HTT at this SNP. Oligonucleotides were delivered at 10 uM, and cells were tested at Day 7. Numbers represent % of HTT remaining, wherein 100.0 would represent 100.000 HTT remaining (0.0% knockdown) and 0.00% would represent 0.00% HTT remaining (100.0% knockdown). Percentage oftubulin (TUBB average) was also determined, wherein 100.0 would represent 100.000 tubulin remaining and 0.00% would represent 0.00% tubulin remaining.

TABLE 25
Activity of certain oligonucleotides.

| Oligonucleotide | % HTT remaining | TUBB Average |
|---|---|---|
| WV-11548 | 87.8 | 108.4 |
| WV-11549 | 73.2 | 118.9 |
| WV-11550 | 96.0 | 131.9 |
| WV-11551 | 84.3 | 140.6 |
| WV-11552 | 70.2 | 92.6 |
| WV-11553 | 77.2 | 101.0 |
| WV-11554 | 78.4 | 90.1 |
| WV-11555 | 121.1 | 74.4 |
| WV-11556 | 72.7 | 116.5 |
| WV-11557 | 79.2 | 134.9 |
| WV-11558 | 102.7 | 115.2 |
| WV-11559 | 77.2 | 112.9 |
| WV-11560 | 95.3 | 100.9 |
| WV-11561 | 91.8 | 105.5 |
| WV-11562 | 70.1 | 114.5 |
| WV-11563 | 78.3 | 83.0 |
| WV-11564 | 93.8 | 109.6 |
| WV-11565 | 87.1 | 124.1 |
| WV-11566 | 91.1 | 125.8 |

Various oligonucleotides to HTT SNP rs362307 were tested for knockdown of HTT in iNeurons (from Pt 100 or Pt 1279), which were homozygous WT HTT at this SNP in both cell types. Oligonucleotides were delivered at 10 uM, and cells were tested at Day 7. Numbers represent % of HTT remaining, wherein 100.0 would represent 100.0% HTT remaining (0.0% knockdown) and 0.0% would represent 0.0% HTT remaining (100.0% knockdown). Percentage of tubulin (TUBB average) was also determined, wherein 100.0 would represent 100.0% tubulin remaining and 0.0% would represent 0.0% tubulin remaining.

TABLE 26
Activity of certain oligonucleotides.

| | % HTT | % HTT | |
|---|---|---|---|
| | remaining | remaining | TUBB |
| Oligonucleotide | (Pt 100) | (Pt 1279) | Average |
| WV-11968 | 74.1 | 80.2 | 100.1 |
| WV-11969 | 67.4 | 61.6 | 118.0 |
| WV-11970 | 61.0 | 57.4 | 116.9 |
| WV-11971 | 52.1 | 54.9 | 123.9 |
| WV-11972 | 49.2 | 45.6 | 123.3 |
| WV-11973 | 55.9 | 55.7 | 110.3 |
| WV-11974 | 60.2 | 58.2 | 108.4 |
| WV-11975 | 74.6 | 67.5 | 91.2 |
| WV-11976 | 50.5 | 53.5 | 106.3 |
| WV-11977 | 60.8 | 57.6 | 117.7 |
| WV-11988 | 72.2 | 75.7 | 105.2 |
| WV-11989 | 66.9 | 58.5 | 96.1 |
| WV-11990 | 61.6 | 59.8 | 121.3 |
| WV-11991 | 70.3 | 66.6 | 109.6 |
| WV-11992 | 59.8 | 67.1 | 109.6 |
| WV-11993 | 63.1 | 55.3 | 93.2 |
| WV-11994 | 58.6 | 71.3 | 92.5 |
| WV-11995 | 63.5 | 69.9 | 88.4 |
| WV-11996 | 54.3 | | 73.8 |
| WV-11997 | 59.4 | 73.1 | 88.4 |
| WV-11998 | 75.7 | 86.6 | 90.2 |
| WV-11999 | 79.9 | 70.9 | 90.1 |
| WV-12000 | 69.4 | 68.9 | 116.7 |
| WV-12001 | 69.0 | 65.4 | 95.5 |
| WV-12002 | 58.6 | No data | 80.5 |
| WV-12003 | 69.3 | No data | 84.8 |
| WV-12004 | 71.1 | No data | 93.6 |
| WV-12005 | 68.4 | No data | 87.3 |
| WV-12006 | 56.9 | No data | 86.7 |
| WV-12007 | 73.5 | No data | 81.4 |

Various oligonucleotides to HTT SNP rs262273 were tested for knockdown of HTT in iNeurons from patient 1279, which are homozygous for mutant rs262273. Oligonucleotides were delivered at 10 uM, and cells were tested at Day 7. Numbers represent % of HTT remaining, wherein 100.0 would represent 100.0% HTT remaining (0.0% knockdown) and 0.0% would represent 0.0% HTT remaining (100.0% knockdown). Percentage oftubulin (TUBB average) was also determined, wherein 100.0 would represent 100.0% tubulin remaining and 0.0% would represent 0.0% tubulin remaining.

TABLE 27
Activity of certain oligonucleotides.

| | | |
|---|---|---|
| Oligonucleotide | % HTT remaining | TUBB Average |

| | | | |
|---|---|---|---|
| WV-12425 | 108.8 | | 86.4 |
| WV-12426 | 91.0 | | 108.9 |
| WV-12427 | 86.4 | | 106.8 |
| WV-12428 | 87.0 | | 101.5 |
| WV-12429 | 93.4 | | 110.6 |
| WV-12430 | 91.3 | | 118.6 |
| WV-12431 | 86.7 | | 113.4 |
| WV-12432 | 104.1 | | 89.1 |
| WV-12433 | 104.7 | | 76.2 |
| WV-12434 | 115.2 | | 100.7 |
| WV-12435 | 101.5 | | 122.5 |
| WV-12436 | 91.6 | | 115.1 |
| WV-12437 | 86.4 | | 121.9 |
| WV-12438 | 89.5 | | 124.2 |
| WV-12258 | 86.7 | | 105.6 |
| WV-12259 | 66.3 | | 99.9 |
| WV-12260 | 74.9 | | 76.4 |
| WV-12261 | 57.3 | | 111.2 |
| WV-12262 | 67.3 | | 93.0 |
| WV-12263 | 93.0 | | 93.2 |
| WV-12264 | 63.0 | | 104.1 |
| WV-12265 | 52.9 | | 97.7 |
| WV-12266 | 88.9 | | 99.2 |
| WV-12267 | 108.5 | | 92.4 |
| WV-12278 | 89.6 | 100.70 | 78.9 |
| WV-12279 | 59.9 | 89.50 | 70.1 |
| WV-12280 | 74.2 | 96.50 | 68.9 |
| WV-12281 | 45.3 | 60.20 | 81.8 |
| WV-12282 | 48.5 | 66.70 | 64.1 |
| WV-12283 | 104.6 | 112.30 | 44.5 |
| WV-12284 | 51.4 | 46.90 | 102.6 |
| WV-12285 | 58.6 | 86.60 | 70.8 |
| WV-12286 | 71.5 | 103.80 | 55.2 |
| WV-12287 | 89.9 | 103.30 | 83.4 |

Various oligonucleotides to HTT SNP rs362307 were tested for knockdown of HTT in]yh′=8]9 from patient 100, which were homozygous WT HTT at this SNP. Oligonucleotides were delivered at 10 uM, and cells were tested at Day 7. Numbers represent % of HTT remaining, wherein 100.0 would represent 100.0% HTT remaining (0.0% knockdown) and 0.0% would represent 0.0% HTT remaining (100.0% knockdown). Percentage oftubulin (TUBB average) was also determined, wherein 100.0 would represent 100.0% tubulin remaining and 0.0% would represent 0.0% tubulin remaining. Negative controls: WV-12889; WV-12890; WV-12891; and WV-12892, which do not target this SNP. Also used was WV-12543, which targets HTT SNP rs362331.

TABLE 28
Activity of certain oligonucleotides.

| | % HTT remaining | | | TUBB Average |
|---|---|---|---|---|
| WV-12288 | | 101.6 | | 125.0 |
| WV-12289 | | 74.7 | | 127.0 |
| WV-12290 | | 84.1 | | 130.1 |
| WV-12291 | | 90.0 | | 125.6 |
| WV-12292 | | 89.5 | | 129.1 |
| WV-12293 | | 102.1 | | 120.6 |
| WV-12294 | | 83.4 | | 114.2 |
| WV-12295 | | 108.8 | | 113.9 |
| WV-12296 | | 93.5 | | 131.8 |
| WV-12297 | | 89.2 | | 133.8 |
| WV-12298 | | 109.6 | | 135.0 |
| WV-12299 | | 105.3 | | 136.7 |
| WV-12300 | | 94.3 | | 132.5 |
| WV-12301 | | 98.3 | | 125.3 |
| WV-12302 | | 91.6 | | 127.5 |
| WV-12889 | | 92.4 | | 141.2 |
| WV-12891 | | 93.6 | | 137.8 |
| WV-12892 | | 93.4 | | 135.5 |
| WV-12543 | 84.2 | 78.6 | 64.9 | 91.8 |
| WV-12544 | 60.3 | 56.9 | 46.9 | 105.6 |
| WV-13625 | | 60.5 | 56.1 | 110.7 |
| WV-13626 | | 42.7 | 33.9 | 109.6 |
| WV-13627 | | 36.8 | 25.4 | 108.6 |
| WV-13628 | | 34.9 | 22.8 | 103.0 |
| WV-13629 | | 43.1 | 36.4 | 106.6 |
| WV-13630 | | 49.8 | 59.2 | 89.2 |
| WV-13631 | | 42.0 | 33.5 | 102.9 |
| WV-13632 | | 69.6 | 55.3 | 84.5 |
| WV-13633 | | 56.0 | 43.1 | 120.2 |
| WV-13634 | | 64.1 | 50.6 | 103.2 |
| WV-13635 | | 67.9 | 52.5 | 100.1 |
| WV-13646 | | 63.1 | 50.5 | 87.2 |
| WV-13647 | | 63.0 | 48.0 | 93.5 |
| WV-13648 | | 52.1 | 44.2 | 93.6 |
| WV-13649 | | 48.4 | 34.2 | 97.4 |
| WV-13650 | | 49.6 | 48.7 | 105.9 |
| WV-13651 | | 55.3 | 49.8 | 111.4 |
| WV-13652 | | 55.9 | 40.4 | 94.3 |
| WV-13653 | | 64.9 | 55.6 | 86.6 |
| WV-13654 | | 62.7 | 61.6 | 87.6 |
| WV-13655 | | 67.8 | 54.9 | 90.0 |
| WV-13656 | | 65.3 | 64.2 | 83.8 |
| WV-13667 | | 73.7 | 61.9 | 87.8 |
| WV-11972 | | 35.6 | 33.9 | 95.2 |

Various HTT oligonucleotides were tested, which target SNP rs362273, but which have different patterns of stereochemistry (e.g., different positions of a phosphorothioate in the Rp configuration, flanked by phosphorothioate in the Sp configuration in the core).
This test of potency was performed in iCell Neurons, which are homozygous for the SNP.
Numbers indicate the 0% of HTT remaining at an oligonucleotide concentration of 10 uM. 100.0 would represent 100.000 HTT remaining (0.0% knockdown) and 0.0 would represent 0.00% HTT remaining (100.0% knockdown). Data from replicates and average are shown.

TABLE 29
Activity of certain oligonucleotides.

| | | |
|---|---|---|
| Oligonucleotide | % remaining @ 10 uM | average |

| | | | |
|---|---|---|---|
| WV-12278 | 101 | 90 | 96 |
| WV-12279 | 89 | 60 | 75 |
| WV-12280 | 97 | 74 | 86 |
| WV-12281 | 60 | 45 | 53 |
| WV-12282 | 67 | 48 | 58 |
| WV-12283 | 112 | 105 | 109 |
| WV-12284 | 47 | 51 | 49 |
| WV-12285 | 87 | 59 | 73 |
| WV-12286 | 104 | 72 | 88 |
| WV-12287 | 103 | 90 | 97 |
| WV-12258 | | 87 | |
| WV-12259 | | 66 | |
| WV-12260 | | 75 | |
| WV-12261 | | 57 | |
| WV-12262 | | 67 | |
| WV-12263 | | 93 | |
| WV-12264 | | 63 | |
| WV-12265 | | 53 | |
| WV-12266 | | 89 | |
| WV-12267 | | 98 | |
| WV-15077 | ~63 | ~47 | ~55 |

HTT oligonucleotides were tested for selectivity in the COS7 cells with the Dual Luciferase assay. The concentration of oligonucleotide used is shown as exp 10 in M. WV-12282 showed an approximately 17-fold selectivity (preferential knockdown of mu HTT compared to wt HTT), and WV-12284 showed an approximately 3-fold selectivity. “wt” indicates knockdown of the wt HTT allele and “mt” indicates knockdown of the mutant HTT allele. Numbers are relative to control.
Numbers indicate the % of HTT remaining (relative to control) at the indicated oligonucleotide concentrations. 1.0 would represent 100.000 HTT remaining (0.0% knockdown) and 0.0 would represent 0.00% HTT remaining (100.0% knockdown). Data are from replicates and average are shown.

TABLE 30
Activity of certain oligonucleotides.

| | |
|---|---|
| Conc. | WV-12282 wt |

| | | | | |
|---|---|---|---|---|
| −7.69897 | 0.674 | 0.725 | 0.713 | 0.814 |
| −8 | 0.803 | 0.852 | 0.902 | 0.983 |
| −8.30103 | 0.800 | 0.889 | 0.905 | 0.966 |
| −8.60206 | 0.950 | 0.959 | 0.981 | 0.851 |
| −8.90309 | 0.923 | 0.979 | 1.045 | 0.967 |
| −9.20412 | 0.998 | 1.001 | 0.998 | 0.973 |
| −9.50515 | 1.030 | 0.985 | 1.031 | 0.999 |
| −9.80618 | 0.868 | 1.008 | 0.864 | 1.178 |
| −10.1072 | 0.835 | 1.036 | 0.898 | 1.069 |

| | |
|---|---|
| Conc. | WV-12282 mt |

| | | | | |
|---|---|---|---|---|
| −7.69897 | 0.083 | 0.093 | 0.093 | 0.080 |
| −8 | 0.234 | 0.189 | 0.262 | 0.205 |
| −8.30103 | 0.418 | 0.357 | 0.496 | 0.353 |
| −8.60206 | 0.647 | 0.501 | 0.675 | 0.521 |
| −8.90309 | 0.744 | 0.671 | 0.811 | 0.654 |
| −9.20412 | 0.839 | 0.801 | 0.955 | 0.722 |
| −9.50515 | 0.902 | 0.839 | 0.918 | 0.861 |
| −9.80618 | 0.879 | 0.953 | 1.005 | 0.912 |
| −10.1072 | 0.940 | 1.188 | 0.981 | 0.998 |

| | |
|---|---|
| Conc. | WV-12284 wt |

| | | | | |
|---|---|---|---|---|
| −7.69897 | 0.374 | 0.357 | 0.406 | 0.361 |
| −8 | 0.695 | 0.615 | 0.716 | 0.728 |
| −8.30103 | 0.928 | 0.863 | 0.921 | 0.972 |
| −8.60206 | 1.071 | 1.019 | 0.879 | 0.932 |
| −8.90309 | 0.976 | 0.957 | 1.002 | 1.039 |
| −9.20412 | 1.058 | 1.068 | 0.971 | 0.998 |
| −9.50515 | 1.131 | 1.027 | 0.986 | 1.142 |
| −9.80618 | 1.028 | 1.123 | 0.931 | 1.250 |
| −10.1072 | 1.016 | 1.050 | 0.948 | 1.012 |

| | |
|---|---|
| Cone. | WV-12284 mt |

| | | | | |
|---|---|---|---|---|
| −7.69897 | 0.093 | 0.102 | 0.092 | 0.088 |
| −8 | 0.271 | 0.228 | 0.298 | 0.286 |
| −8.30103 | 0.523 | 0.451 | 0.470 | 0.483 |
| −8.60206 | 0.678 | 0.642 | 0.711 | 0.665 |
| −8.90309 | 0.829 | 0.783 | 0.993 | 0.729 |
| −9.20412 | 0.855 | 0.909 | 0.926 | 0.897 |
| −9.50515 | 0.930 | 0.919 | 0.933 | 0.918 |
| −9.80618 | 0.921 | 0.950 | 1.108 | 0.987 |
| −10.1072 | 0.978 | 1.023 | 1.003 | 0.999 |

Various HTT oligonucleotides were tested for knockdown of HTT.
Various oligonucleotides target SNP rs362273, but comprise different 2′-sugar modifications in the 5′ and 3′ wings (wherein some have an asymmetric format), and different patterns of stereochemistry in the core region.
This test of potency was performed in iCell Neurons, which are homozygous for the SNP.
Numbers indicate the % of HTT remaining (relative to control) at an oligonucleotide concentration of 10 uM. 100.0 would represent 100.0% HTT remaining (0.0% knockdown) and 0.0 would represent 0.0% HTT remaining (100.0% knockdown). Data are from replicates and average are shown.

TABLE 31
Activity of certain oligonucleotides.

| Oligonucleotide | % HTT remaining 10 10 uM |
|---|---|
| WV-14092 | 68 |
| WV-14093 | 76 |
| WV-14094 | 82 |
| WV-14095 | 75 |
| WV-14096 | 61 |
| WV-14097 | 73 |
| WV-14098 | 59 |
| WV-14099 | 59 |
| WV-14100 | 55 |
| WV-14101 | 54 |
| WV-12425 | 109 |
| WV-12426 | 91 |
| WV-12427 | 86 |
| WV-12428 | 87 |
| WV-12429 | 93 |
| WV-12430 | 91 |
| WV-12431 | 87 |
| WV-12432 | 104 |
| WV-12433 | 105 |
| WV-12434 | 115 |
| WV-12435 | 102 |
| WV-12436 | 92 |
| WV-12437 | 86 |
| WV-12438 | 90 |
| WV-12282 | 67/48, ave. 58% |

Various HTT oligonucleotides which comprise one or more non-negatively charged internucleotidic linkage were tested. This test to determine IC50 was performed in iCell Neurons, which are homozygous for the SNP.

TABLE 32
Activity of certain oligonucleotides.

| Oligonucleotide | IC50 nM | |
|---|---|---|
| WV-17776 | 474 | nM |
| WV-17777 | 3301 | nM |
| WV-17778 | 1186 | nM |
| WV-17779 | 1317 | nM |
| WV-17780 | 1504 | nM |
| WV-17781 | 992 | nM |
| WV-17782 | 467 | nM |
| WV-14914 | 861 | nM |
| WV-14915 | 3970 | nM |
| WV-15079 | 4042 | nM |
| WV-15080 | 696 | nM |
| WV-17783 | IC50 >10 | uM |
| WV-17784 | IC50 >10 | uM |
| WV-17785 | IC50 >10 | uM |
| WV-17786 | IC50 >10 | uM |
| WV-17787 | IC50 >10 | uM |
| WV-17788 | IC50 >10 | uM |
| WV-17789 | IC50 >10 | uM |
| WV-17790 | IC50 >10 | uM |
| WV-17791 | IC50 >10 | uM |
| WV-17792 | IC50 >10 | uM |
| WV-17793 | IC50 >10 | uM |
| WV-17794 | IC50 >10 | uM |
| WV-17795 | IC50 >10 | uM |
| WV-17796 | IC50 >10 | uM |
| WV-17797 | IC50 >10 | uM |
| WV-17798 | IC50 >10 | uM |
| WV-17799 | IC50 >10 | uM |
| WV-17780 | IC50 >10 | uM |

Various HTT oligonucleotides were tested for selectivity in the Dual Luciferase assay.
Cells were transfected with reporter plasmid and ASO starting at 20 nM with an 11-point 2-fold dilution series. Data were collected 2 days later. IC50 was derived from curve fits on next slide. Molecules generally very similar to each other, with highest fold change in WV-17782, as well as >75% KD of mutant and only 25% KD of wt at 5 nM.
In this table: Numbers indicate the % of HTT knockdown (relative to control) at an oligonucleotide concentration of 5 nM. 0.0 would represent 100.00% HTT remaining (0.0% knockdown) and 100.0 would represent 0.00% HTT remaining (100.0% knockdown). Data are from replicates and average are shown.

TABLE 33
Activity of certain oligonucleotides.

| | IC50 | | | % KD 5 nM | |
|---|---|---|---|---|---|
| | wt IC50 | mt IC50 | fold change | wt | mt |
| WV-17776 | 1.029E−08 | 1.952E−09 | 5.3 | 31 | 74 |
| WV-17777 | 1.13E−08 | 1.49E−09 | 7.6 | 31 | 84 |
| WV-17778 | 1.033E−08 | 1.71E−09 | 6 | 30 | 83 |
| WV-17779 | 1.018E−08 | 1.549E−09 | 6.6 | 28 | 85 |
| WV-17780 | 9.634E−09 | 1.139E−09 | 8.5 | 36 | 88 |
| WV-17781 | 1.13E−08 | 1.53E−09 | 7.4 | 26 | 85 |
| WV-17782 | 1.141E−08 | 1.311E−09 | 8.7 | 25 | 82 |
| WV-15078 | 1.562E−09 | 9.127E−10 | 1.7 | 82 | 89 |
| WV-14914 | 3.72e−8 | 3.27e−9 | 11.5 | | |

Various HTT oligonucleotides were tested, in which the SNP was walked through various positions in the oligonucleotide sequence.
Numbers indicate the % of HTT remaining (relative to control) at an oligonucleotide concentration of 10 uM. Numbers are approximate. 100.0 would represent 100.0% HTT remaining (0.0% knockdown) and 0.0 would represent 0.0% HTT remaining (100.0% knockdown). Data are from replicates and average are shown.

TABLE 34
Activity of certain oligonucleotides.

| ASO | % HTT remaining at 10 uM |
|---|---|
| WV-14059 | 85 |
| WV-14060 | 93 |
| WV-14061 | 85 |
| WV-14062 | 78 |
| WV-14063 | 94 |
| WV-14064 | 92 |
| WV-14065 | 84 |
| WV-14066 | 90 |
| WV-14067 | 95 |
| WV-14068 | 89 |
| WV-14069 | 76 |
| WV-14070 | 98 |
| WV-14071 | 97 |
| WV-14072 | 86 |
| WV-14080 | 90 |
| WV-14081 | 84 |
| WV-14082 | 79 |
| WV-14083 | 74 |
| WV-14084 | 101 |
| WV-14085 | 88 |
| WV-14086 | 87 |

Various oligonucleotides were tested for activity in vitro.
Numbers indicate the % of HTT remaining (relative to control) at an oligonucleotide at the indicated concentrations. The concentration of oligonucleotide used is shown as exp 10 in M. 1.000 would represent 100.00% HTT remaining (0.0% knockdown) and 0.0 would represent 0.00% HTT remaining (100.0% knockdown). Data are from replicates and average are shown.

TABLE 35
Activity of certain oligonucleotides.

| | |
|---|---|
| Conc | WV-14914 wt |

| | | | | |
|---|---|---|---|---|
| −7.69897 | 0.707 | 0.787 | 0.611 | 0.693 |
| −8 | 0.936 | 0.872 | 0.879 | 0.874 |
| −8.30103 | 0.968 | 1.033 | 0.959 | 0.634 |
| −8.60206 | 1.057 | 0.962 | 1.033 | 0.772 |
| −8.90309 | 0.907 | 0.946 | 1.105 | 0.886 |
| −9.20412 | 0.966 | 0.897 | 1.085 | 0.854 |
| −9.50515 | 0.916 | 0.839 | 1.103 | 1.056 |
| −9.80618 | 0.900 | 0.954 | 1.040 | 0.916 |
| −10.1072 | 0.941 | 1.047 | 0.998 | 1.058 |

| | |
|---|---|
| Conc | WV-17776 wt |

| | | | | |
|---|---|---|---|---|
| −7.69897 | 0.270 | 0.299 | 0.317 | 0.322 |
| −8 | 0.545 | 0.564 | 0.535 | 0.546 |
| −8.30103 | 0.633 | 0.658 | 0.738 | 0.712 |
| −8.60206 | 0.805 | 0.741 | 0.743 | 0.861 |
| −8.90309 | 0.887 | 0.849 | 0.797 | 0.868 |
| −9.20412 | 0.972 | 1.041 | 0.957 | 0.901 |
| −9.50515 | 0.896 | 0.905 | 0.898 | 1.000 |
| −9.80618 | 1.074 | 0.946 | 1.067 | 0.861 |
| −10.1072 | 0.968 | 0.985 | 0.959 | 0.936 |
| −10.4082 | 1.043 | 1.044 | 0.977 | 0.991 |
| −10.7093 | 1.154 | 1.004 | 1.007 | 0.983 |

| | |
|---|---|
| Conc | WV-17777 wt |

| | | | | |
|---|---|---|---|---|
| −7.69897 | 0.227 | 0.241 | 0.299 | 0.325 |
| −8 | 0.554 | 0.537 | 0.543 | 0.610 |
| −8.30103 | 0.643 | 0.627 | 0.753 | 0.740 |
| −8.60206 | 0.827 | 0.789 | 0.873 | 0.880 |
| −8.90309 | 0.932 | 0.977 | 0.951 | 0.965 |
| −9.20412 | 1.051 | 1.028 | 0.884 | 0.930 |
| −9.50515 | 0.925 | 0.920 | 0.908 | 0.966 |
| −9.80618 | 0.983 | 1.015 | 0.999 | 0.879 |
| −10.1072 | 0.894 | 1.061 | 0.955 | 0.883 |
| −10.4082 | 0.928 | 0.970 | 0.914 | 0.939 |
| −10.7093 | 1.041 | 1.025 | 1.010 | 0.999 |

| | |
|---|---|
| Conc | WV-17778 wt |

| | | | | |
|---|---|---|---|---|
| −7.69897 | 0.223 | 0.251 | 0.255 | 0.309 |
| −8 | 0.467 | 0.508 | 0.560 | 0.533 |
| −8.30103 | 0.688 | 0.661 | 0.704 | 0.747 |
| −8.60206 | 0.830 | 0.875 | 0.880 | 0.912 |
| −8.90309 | 0.962 | 0.907 | 0.890 | 1.015 |
| −9.20412 | 1.092 | 1.047 | 1.020 | 0.911 |
| −9.50515 | 0.934 | 0.950 | 0.973 | 0.982 |
| −9.80618 | 1.078 | 1.012 | 0.898 | 0.963 |
| −10.1072 | 0.901 | 0.909 | 0.952 | 0.854 |
| −10.4082 | 1.035 | 0.990 | 0.988 | 0.920 |
| −10.7093 | 1.058 | 1.031 | 1.010 | 1.078 |

| | |
|---|---|
| Conc | WV-17779 wt |

| | | | | |
|---|---|---|---|---|
| −7.69897 | 0.236 | 0.288 | 0.266 | 0.297 |
| −8 | 0.467 | 0.547 | 0.538 | 0.626 |
| −8.30103 | 0.710 | 0.683 | 0.764 | 0.728 |
| −8.60206 | 0.829 | 0.890 | 0.797 | 0.862 |
| −8.90309 | 0.941 | 0.808 | 0.936 | 0.918 |
| −9.20412 | 1.013 | 1.042 | 0.875 | 0.890 |
| −9.50515 | 0.972 | 0.829 | 0.978 | 1.013 |
| −9.80618 | 0.959 | 1.020 | 0.959 | 0.890 |
| −10.1072 | 1.090 | 0.977 | 0.982 | 0.969 |
| −10.4082 | 1.030 | 1.048 | 1.041 | 1.075 |
| −10.7093 | 1.078 | 1.032 | 1.215 | 0.940 |

| | |
|---|---|
| Conc | WV-17780 wt |

| | | | | |
|---|---|---|---|---|
| −7.69897 | 0.268 | 0.276 | 0.256 | 0.303 |
| −8 | 0.492 | 0.457 | 0.524 | 0.563 |
| −8.30103 | 0.527 | 0.600 | 0.720 | 0.726 |
| −8.60206 | 0.760 | 0.816 | 0.723 | 0.791 |
| −8.90309 | 0.885 | 0.863 | 0.959 | 0.946 |
| −9.20412 | 0.931 | 0.920 | 0.927 | 0.937 |
| −9.50515 | 0.771 | 0.876 | 0.908 | 0.982 |
| −9.80618 | 0.954 | 0.896 | 0.948 | 1.020 |
| −10.1072 | 0.923 | 0.855 | 0.906 | 0.984 |
| −10.4082 | 1.020 | 0.923 | 0.928 | 0.935 |
| −10.7093 | 1.165 | 1.045 | 0.988 | 1.105 |

| | |
|---|---|
| Conc | WV-17781 wt |

| | | | | |
|---|---|---|---|---|
| −7.69897 | 0.245 | 0.297 | 0.275 | 0.304 |
| −8 | 0.527 | 0.488 | 0.529 | 0.625 |
| −8.30103 | 0.663 | 0.737 | 0.807 | 0.748 |
| −8.60206 | 0.896 | 0.816 | 0.864 | 0.893 |
| −8.90309 | 0.997 | 0.974 | 0.945 | 0.845 |
| −9.20412 | 1.004 | 0.939 | 0.964 | 0.953 |
| −9.50515 | 0.888 | 0.996 | 0.907 | 1.021 |
| −9.80618 | 1.023 | 0.973 | 1.042 | 0.890 |
| −10.1072 | 0.948 | 1.005 | 1.001 | 0.974 |
| −10.4082 | 1.071 | 0.977 | 0.882 | 0.996 |
| −10.7093 | 1.074 | 0.965 | 1.055 | 0.940 |

| | |
|---|---|
| Conc | WV-17782 wt |

| | | | | |
|---|---|---|---|---|
| −7.69897 | 0.378 | 0.280 | 0.346 | 0.327 |
| −8 | 0.473 | 0.608 | 0.552 | 0.599 |
| −8.30103 | 0.861 | 0.637 | 0.784 | 0.717 |
| −8.60206 | 0.667 | 0.777 | 0.884 | 0.820 |
| −8.90309 | 0.815 | 0.945 | 0.989 | 0.832 |
| −9.20412 | 0.944 | 0.932 | 0.776 | 0.929 |
| −9.50515 | 0.937 | 1.112 | 0.811 | 0.938 |
| −9.80618 | 1.039 | 0.925 | 0.931 | 1.056 |
| −10.1072 | 0.854 | 1.042 | 1.020 | 1.017 |
| −10.4082 | 1.309 | 0.988 | 0.889 | 1.053 |
| −10.7093 | 0.990 | 1.087 | 1.060 | 1.023 |

| | |
|---|---|
| Conc | WV-15078 (+ control) wt |

| | | | | |
|---|---|---|---|---|
| −7.69897 | 0.035 | 0.032 | 0.035 | 0.040 |
| −8 | 0.072 | 0.082 | 0.076 | 0.083 |
| −8.30103 | 0.173 | 0.145 | 0.213 | 0.200 |
| −8.60206 | 0.328 | 0.385 | 0.434 | 0.333 |
| −8.90309 | 0.567 | 0.540 | 0.535 | 0.564 |
| −9.20412 | 0.741 | 0.729 | 0.773 | 0.760 |
| −9.50515 | 0.948 | 0.772 | 0.894 | 0.796 |
| −9.80618 | 0.854 | 0.815 | 0.815 | 0.874 |
| −10.1072 | 0.972 | 0.915 | 0.984 | 0.877 |
| −10.4082 | 0.921 | 0.874 | 0.830 | 0.915 |
| −10.7093 | 1.084 | 0.875 | 1.046 | 0.889 |

| | |
|---|---|
| Conc | WV-14914 mt |

| | | | | |
|---|---|---|---|---|
| −7.69897 | 0.098 | 0.107 | 0.096 | 0.101 |
| −8 | 0.204 | 0.219 | 0.233 | 0.187 |
| −8.30103 | 0.348 | 0.386 | 0.382 | 0.413 |
| −8.60206 | 0.637 | 0.504 | 0.613 | 0.551 |
| −8.90309 | 0.657 | 0.675 | 0.774 | 0.716 |
| −9.20412 | 0.874 | 0.812 | 0.878 | 0.755 |
| −9.50515 | 0.815 | 0.805 | 0.970 | 0.875 |
| −9.80618 | 0.852 | 0.889 | 0.922 | 1.066 |
| −10.1072 | 0.898 | 0.997 | 0.993 | 1.034 |

| | |
|---|---|
| Conc | WV-17776 mutant |

| | | | | |
|---|---|---|---|---|
| −7.69897 | 0.026 | 0.032 | 0.036 | 0.032 |
| −8 | 0.087 | 0.112 | 0.086 | 0.157 |
| −8.30103 | 0.234 | 0.220 | 0.278 | 0.291 |
| −8.60206 | 0.484 | 0.375 | 0.358 | 0.392 |
| −8.90309 | 0.738 | 0.687 | 0.645 | 0.654 |
| −9.20412 | 0.969 | 0.774 | 0.734 | 0.709 |
| −9.50515 | 0.846 | 0.866 | 0.982 | 0.801 |
| −9.80618 | 0.887 | 0.968 | 0.906 | 0.962 |
| −10.1072 | 0.952 | 0.881 | 0.905 | 0.898 |
| −10.4082 | 1.126 | 0.953 | 0.992 | 0.799 |
| −10.7093 | 1.063 | 0.918 | 0.982 | 0.971 |

| | |
|---|---|
| Conc | WV-17777 mutant |

| | | | | |
|---|---|---|---|---|
| −7.69897 | 0.018 | 0.018 | 0.030 | 0.038 |
| −8 | 0.040 | 0.049 | 0.050 | 0.062 |
| −8.30103 | 0.135 | 0.149 | 0.174 | 0.170 |
| −8.60206 | 0.369 | 0.325 | 0.326 | 0.337 |
| −8.90309 | 0.620 | 0.613 | 0.534 | 0.603 |
| −9.20412 | 0.816 | 0.658 | 0.625 | 0.746 |
| −9.50515 | 0.827 | 0.691 | 0.832 | 0.751 |
| −9.80618 | 0.864 | 0.954 | 0.782 | 0.794 |
| −10.1072 | 0.890 | 0.862 | 0.853 | 0.890 |
| −10.4082 | 1.026 | 0.888 | 0.789 | 0.923 |
| −10.7093 | 1.057 | 0.891 | 0.944 | 0.908 |

| | |
|---|---|
| Conc | WV-17778 mutant |

| | | | | |
|---|---|---|---|---|
| −7.69897 | 0.026 | 0.024 | 0.034 | 0.030 |
| −8 | 0.059 | 0.054 | 0.057 | 0.057 |
| −8.30103 | 0.159 | 0.147 | 0.189 | 0.181 |
| −8.60206 | 0.400 | 0.337 | 0.307 | 0.337 |
| −8.90309 | 0.596 | 0.585 | 0.550 | 0.660 |
| −9.20412 | 0.933 | 0.728 | 0.610 | 0.727 |
| −9.50515 | 0.689 | 0.756 | 0.790 | 0.827 |
| −9.80618 | 0.924 | 0.818 | 0.803 | 0.950 |
| −10.1072 | 0.851 | 0.762 | 0.784 | 0.847 |
| −10.4082 | 0.920 | 0.857 | 0.785 | 0.763 |
| −10.7093 | 1.003 | 0.971 | 0.784 | 0.998 |

| | |
|---|---|
| Conc | WV-17779 mutant |

| | | | | |
|---|---|---|---|---|
| −7.69897 | 0.018 | 0.017 | 0.019 | 0.018 |
| −8 | 0.037 | 0.053 | 0.038 | 0.049 |
| −8.30103 | 0.120 | 0.159 | 0.151 | 0.175 |
| −8.60206 | 0.322 | 0.362 | 0.324 | 0.346 |
| −8.90309 | 0.626 | 0.637 | 0.479 | 0.565 |
| −9.20412 | 0.942 | 0.662 | 0.728 | 0.704 |
| −9.50515 | 0.694 | 0.785 | 0.777 | 0.739 |
| −9.80618 | 0.840 | 0.773 | 0.875 | 0.949 |
| −10.1072 | 0.854 | 0.872 | 0.848 | 0.824 |
| −10.4082 | 0.916 | 0.893 | 0.828 | 0.809 |
| −10.7093 | 0.930 | 0.874 | 0.988 | 1.008 |

| | |
|---|---|
| Conc | WV-17780 mutant |

| | | | | |
|---|---|---|---|---|
| −7.69897 | 0.015 | 0.015 | 0.024 | 0.018 |
| −8 | 0.040 | 0.031 | 0.043 | 0.050 |
| −8.30103 | 0.093 | 0.134 | 0.100 | 0.149 |
| −8.60206 | 0.253 | 0.299 | 0.245 | 0.303 |
| −8.90309 | 0.503 | 0.570 | 0.426 | 0.559 |
| −9.20412 | 0.722 | 0.648 | 0.675 | 0.756 |
| −9.50515 | 0.703 | 0.726 | 0.813 | 0.731 |
| −9.80618 | 0.888 | 0.812 | 0.922 | 0.980 |
| −10.1072 | 0.806 | 0.896 | 0.943 | 0.920 |
| −10.4082 | 0.959 | 0.951 | 0.806 | 0.864 |
| −10.7093 | 1.147 | 0.909 | 0.919 | 1.001 |

| | |
|---|---|
| Conc | WV-17781 mutant |

| | | | | |
|---|---|---|---|---|
| −7.69897 | 0.026 | 0.025 | 0.031 | 0.041 |
| −8 | 0.055 | 0.040 | 0.056 | 0.076 |
| −8.30103 | 0.111 | 0.125 | 0.198 | 0.176 |
| −8.60206 | 0.329 | 0.396 | 0.330 | 0.383 |
| −8.90309 | 0.675 | 0.683 | 0.525 | 0.541 |
| −9.20412 | 0.822 | 0.702 | 0.821 | 0.754 |
| −9.50515 | 0.798 | 0.816 | 0.759 | 0.827 |
| −9.80618 | 0.948 | 0.809 | 0.851 | 0.958 |
| −10.1072 | 0.755 | 0.913 | 0.810 | 1.016 |
| −10.4082 | 1.104 | 0.908 | 0.854 | 0.834 |
| −10.7093 | 0.993 | 0.968 | 1.086 | 0.988 |

| | |
|---|---|
| Conc | WV-17782 mutant |

| | | | | |
|---|---|---|---|---|
| −7.69897 | 0.014 | 0.019 | 0.021 | 0.022 |
| −8 | 0.064 | 0.068 | 0.079 | 0.078 |
| −8.30103 | 0.162 | 0.206 | 0.184 | 0.189 |
| −8.60206 | 0.411 | 0.361 | 0.332 | 0.311 |
| −8.90309 | 0.477 | 0.569 | 0.582 | 0.622 |
| −9.20412 | 0.821 | 0.877 | 0.743 | 0.692 |
| −9.50515 | 0.966 | 0.844 | 0.728 | 0.797 |
| −9.80618 | 0.972 | 0.912 | 0.927 | 0.912 |
| −10.1072 | 0.939 | 1.241 | 0.842 | 1.047 |
| −10.4082 | 1.053 | 1.065 | 0.921 | 0.986 |
| −10.7093 | 1.160 | 1.008 | 0.984 | 0.990 |

| | |
|---|---|
| Conc | WV-15078 (+ control) mutant |

| | | | | |
|---|---|---|---|---|
| −7.69897 | 0.012 | 0.024 | 0.023 | 0.020 |
| −8 | 0.046 | 0.045 | 0.043 | 0.060 |
| −8.30103 | 0.096 | 0.112 | 0.120 | 0.130 |
| −8.60206 | 0.237 | 0.292 | 0.245 | 0.238 |
| −8.90309 | 0.465 | 0.410 | 0.534 | 0.497 |
| −9.20412 | 0.621 | 0.586 | 0.601 | 0.667 |
| −9.50515 | 0.722 | 0.733 | 0.787 | 0.742 |
| −9.80618 | 0.826 | 0.822 | 0.760 | 0.864 |
| −10.1072 | 0.872 | 0.978 | 0.912 | 0.822 |
| −10.4082 | 0.944 | 1.042 | 0.889 | 0.896 |
| −10.7093 | 1.179 | 0.963 | 0.924 | 1.091 |

Various HTT oligonucleotides which comprise various patterns of backbone stereochemistry in the core, and one or more non-negatively charged internucleotidic linkage were tested. This test to determine IC50 was performed in iCell Neurons, which are homozygous for the SNP.

TABLE 36
Activity of certain oligonucleotides.

| ASO | IC50 nM | |
|---|---|---|
| WV-17776 | 474 | nM |
| WV-17777 | 3301 | nM |
| WV-17778 | 1186 | nM |
| WV-17779 | 1317 | nM |
| WV-17780 | 1504 | nM |
| WV-17781 | 992 | nM |
| WV-17782 | 467 | nM |
| WV-17783 | IC50 >10 | uM |
| WV-17784 | IC50 >10 | uM |
| WV-17785 | IC50 >10 | uM |
| WV-17786 | IC50 >10 | uM |
| WV-17787 | IC50 >10 | uM |
| WV-17788 | IC50 >10 | uM |
| WV-17789 | IC50 >10 | uM |
| WV-17790 | IC50 >10 | uM |
| WV-17791 | IC50 >10 | uM |
| WV-17792 | IC50 >10 | uM |
| WV-17793 | IC50 >10 | uM |
| WV-17794 | IC50 >10 | uM |
| WV-17795 | IC50 >10 | uM |
| WV-17796 | IC50 >10 | uM |
| WV-17797 | IC50 >10 | uM |
| WV-17798 | IC50 >10 | uM |
| WV-17799 | IC50 >10 | uM |
| WV-17780 | IC50 >10 | uM |

Various HTT oligonucleotides were test in vivo in animals for knockdown. Numbers present here represent relative level of HTT (hHTT/mHPRT1/PBS-treated). Numbers are for levels in hippocampus, as determined using 174 Taq probe.
Numbers indicate the % of HTT remaining (relative to control). Numbers are approximate. 100.0 would represent 100.0% HTT remaining (0.0% knockdown) and 0.0 would represent 0.0% HTT remaining (100.0% knockdown). Data are from replicates and averages are shown.

TABLE 37
Activity of certain oligonucleotides.

| | |
|---|---|
| PBS Wk 1 | 100 |
| PBS Wk 2 | 100 |
| WV-9679 Wk1 | 85 |
| WV-9679 Wk 2 | 50 |
| WV-15080 Wk 1 | 60 |
| WV-15080 Wk 2 | 92 |
| WV-14914 Wk 1 | 80 |
| WV-14914 Wk 2 | 60 |
| WV-12282 Wk 1 | 90 |
| WV-12281 WK 2 | 96 |
| WV-12284 Wk 1 | 84 |
| WV-12284 Wk 2 | 82 |

Various HTT oligonucleotides were tested in vitro.
Numbers indicate the % of HTT remaining (relative to control) at an oligonucleotide concentration of 10 uM in neurons heterozygous for this SNP. 1.00 would represent 100.00% HTT remaining (0.0% knockdown) and 0.0 would represent 0.00% HTT remaining (100.0% knockdown).

TABLE 38
Activity of certain oligonucleotides.

| | | Oligonucleotides | | | | |
|---|---|---|---|---|---|---|
| | | WV- | WV- | WV- | WV- | WV- |
| | | 15078 | 12282 | 12283 | 14914 | 15080 |
| Selectivity | A allele | 0.22 | 0.41 | 0.52 | 0.23 | 0.28 |
| | G allele | 0.59 | 1.29 | 0.72 | 0.82 | 0.34 |

The IC50 of various oligonucleotides was determined in vitro.
This test of potency was performed in iCell Neurons. The IC50 in nM is presented below.

TABLE 39
Activity of certain oligonucleotides.

| Oligonucleotide | IC50 (nM) | Oligonucleotide | IC50 (nM) |
|---|---|---|---|
| WV-9679 | 5481 | WV-17781 | 1030 |
| WV-15078 | 4534 | WV-19820 | 2455 |
| WV-12282 | 4068 | WV-19821 | 1701 |
| WV-12284 | 4016 | WV-19822 | 4120 |
| WV-14914 | 861 | WV-19823 | 2030 |
| WV-14915 | 3970 | WV-19824 | 970 |
| WV-15079 | 4043 | WV-19825 | 1490 |
| WV-15080 | 666 | WV-19838 | 1410 |
| WV-15077 | 3902 | WV-19839 | 2390 |
| WV-17782 | 830 | WV-19840 | 1190 |
| WV-16214 | 4831 | WV-19856 | 2570 |
| WV-16215 | 2027 | WV-19857 | 20000 |
| WV-16216 | 1885 | WV-19858 | 3815 |
| WV-16217 | 6470 | WV-19859 | 4720 |
| WV-16218 | 12345 | WV-19860 | 8788 |
| WV-17777 | 8830 | WV-19861 | 2098 |
| WV-17778 | 3420 | WV-19862 | 20000 |
| WV-17779 | 1440 | WV-19863 | 5859 |
| WV-17780 | 2090 | | |

Various HTT oligonucleotides were tested in vitro.
Cells used were a homozygous HD patient cell line: ND40536-1 (MSN or medium spiny neuron), which is homozygous for rs362273 and heterozygous/phased for rs362307; the CAG repeat is on the same chromosomal strand as (in phase with) SNP1, rs362307.
Medium spiny neurons were generated by BrainXell, thawed according to protocol, and treated 7 d post-thaw. Additional media was added id post-treatment; RNA extracted 7 d post-treatment.
Evaluated by qPCR as part of assay optimization for ND40536-1 neurons.
WV-14914 targets HTT SNP rs362273. WV-9679 targets HTT but not at this SNP. WV-12890 targets LUC (luciferase). Numbers represent HTT mRNA expression (after knockdown), normalized to vehicle, measured by qPCR in ND40536-1 MSNs, using 48 well plates, treated Day 7, for 7 days. In tables 39 to 41: 1.00 would represent 100.0% HTT remaining (0.0% knockdown) and 0.0 would represent 0.0% HTT remaining (100.0% knockdown).

TABLE 40A AND 40B
Activity of certain oligonucleotides.

| | | | |
|---|---|---|---|
| Conc. | | | |
| (log 10M) | WV-9679 | WV-14914 | WV-12890 |

| | | | | | | |
|---|---|---|---|---|---|---|
| −6.505 | 0.786 | 0.949 | 0.837 | 0.787 | 0.684 | 0.740 |
| −6.204 | 0.827 | 0.855 | 0.775 | 0.784 | 0.948 | 1.173 |
| −5.903 | 0.781 | 0.619 | 0.902 | 0.804 | 0.847 | 1.162 |
| −5.602 | 0.767 | 0.665 | 0.371 | 0.690 | 0.933 | 0.953 |
| −5.301 | 0.729 | 0.866 | 0.275 | 0.396 | 1.170 | 0.775 |
| −5.000 | 0.401 | 0.604 | 0.146 | 0.113 | 0.992 | 0.916 |

Various HTT oligonucleotides were tested in vitro.
In Tables 40A and 41A: Allele-specific knockdown was tested using MiSeq/Tagman total MRNA assay, with iCell Neurons from Patient 1. 7 day treatment was used. Numbers represent individual allele (G or A) remaining, normalized to NTC.
In Tables 40B and 41B: Allele-specific knockdown was tested using TaqMan genotyping/total mRNA assay, with iCell Neurons from Patient 1. 7 day treatment was used. Numbers represent individual allele (G or A) remaining, normalized to NTC.
WV-12282, WV-12283, WV-14914, WV-15078, and WV-15080 all target HTT SNP rs362273. NTC, non-targeting control.

TABLE 40A

| | | wild type (G) | | | mutant (A) | | | |
|---|---|---|---|---|---|---|---|---|
| NTC 10 uM | | 0.542 | 1.429 | 1.028 | 1.153 | 1.096 | 0.751 | |
| 0.1 | uM | 1.326 | 1.253 | 0.973 | 0.635 | 0.618 | 0.616 | WV-12282 |
| 1 | uM | 1.396 | 1.474 | 1.370 | 0.560 | 0.472 | 0.513 | |
| 10 | uM | 2.414 | 0.964 | 1.461 | 0.322 | 0.140 | 0.300 | |
| 0.1 | uM | 0.679 | 1.281 | 1.221 | 0.415 | 0.699 | 0.357 | WV-12283 |
| 1 | uM | 1.897 | 1.238 | 1.211 | 0.573 | 0.620 | 0.581 | |
| 10 | uM | 1.135 | 1.288 | 1.432 | 0.308 | 0.191 | 0.167 | |
| 0.1 | uM | 0.792 | 0.974 | 1.776 | 0.544 | 0.364 | 0.646 | WV-14914 |
| 1 | uM | 1.254 | 1.063 | 1.021 | 0.451 | 0.410 | 0.254 | |
| 10 | uM | 1.126 | 0.883 | 1.500 | 0.074 | 0.066 | 0.030 | |
| 0.1 | uM | 1.106 | 1.464 | 1.479 | 0.563 | 0.688 | 0.636 | WV-15078 |
| 1 | uM | 1.830 | 1.000 | 1.781 | 0.511 | 0.342 | 0.335 | |
| 10 | uM | 1.100 | 0.815 | 1.056 | 0.015 | 0.043 | 0.017 | |
| 0.1 | uM | 1.184 | 1.064 | 1.563 | 0.711 | 0.819 | 0.607 | WV-15080 |
| 1 | uM | 1.013 | 1.113 | 1.814 | 0.412 | 0.372 | 0.555 | |
| 10 | uM | 0.951 | 0.695 | 0.576 | 0.039 | 0.179 | 0.003 | |

TABLE 40B
Activity of certain oligonucleotides.

| | | wild type (G) | | | mutant (A) | | | |
|---|---|---|---|---|---|---|---|---|
| NTC 10 uM | | 0.877 | 1.216 | 0.908 | 0.980 | 1.208 | 0.812 | |
| 0.1 | uM | 1.097 | 0.995 | 0.804 | 0.747 | 0.747 | 0.703 | WV-12282 |
| 1 | uM | 1.127 | 1.124 | 1.065 | 0.692 | 0.645 | 0.664 | |
| 10 | uM | 2.038 | 0.554 | 1.273 | 0.486 | 0.351 | 0.379 | |
| 0.1 | uM | 0.524 | 0.998 | 0.599 | 0.496 | 0.844 | 0.685 | WV-12283 |
| 1 | uM | 1.211 | 0.975 | 0.987 | 0.926 | 0.754 | 0.693 | |
| 10 | uM | 0.736 | 0.503 | 0.907 | 0.513 | 0.605 | 0.432 | |
| 0.1 | uM | 0.743 | 0.638 | 1.313 | 0.565 | 0.537 | 0.879 | WV-14914 |
| 1 | uM | 1.213 | 0.961 | 0.607 | 0.457 | 0.453 | 0.468 | |
| 10 | uM | 0.823 | 0.565 | 1.062 | 0.220 | 0.225 | 0.243 | |
| 0.1 | uM | 0.864 | 1.154 | 1.086 | 0.686 | 0.844 | 0.836 | WV-15078 |
| 1 | uM | 1.718 | 0.886 | 1.378 | 0.545 | 0.391 | 0.528 | |
| 10 | uM | 0.674 | 0.474 | 0.635 | 0.229 | 0.217 | 0.229 | |
| 0.1 | uM | 1.000 | 1.157 | 1.165 | 0.803 | 0.761 | 0.809 | WV-15080 |
| 1 | uM | 0.926 | 0.939 | 1.639 | 0.448 | 0.453 | 0.625 | |
| 10 | uM | 0.413 | 0.418 | 0.191 | 0.319 | 0.322 | 0.205 | |

### Table 41A and 41B. Activity of Certain Oligonucleotides.

Various HTT oligonucleotides were tested in vitro.
WV-12282, WV-12283, WV-14914, WV-15078, and WV-15080 all target HTT SNP rs362273.

TABLE 41A

| | | wild type (G) | | | mutant (A) | | | |
|---|---|---|---|---|---|---|---|---|
| NTC 10 uM | | 0.187 | 0.492 | 0.354 | 0.756 | 0.719 | 0.493 | |
| 0.1 | uM | 0.456 | 0.431 | 0.335 | 0.417 | 0.405 | 0.404 | WV-12282 |
| 1 | uM | 0.480 | 0.507 | 0.471 | 0.367 | 0.309 | 0.337 | |
| 10 | uM | 0.830 | 0.331 | 0.502 | 0.211 | 0.092 | 0.197 | |
| 0.1 | uM | 0.233 | 0.440 | 0.420 | 0.272 | 0.459 | 0.234 | WV-12283 |
| 1 | uM | 0.652 | 0.426 | 0.416 | 0.376 | 0.407 | 0.381 | |
| 10 | uM | 0.390 | 0.443 | 0.492 | 0.202 | 0.125 | 0.109 | |
| 0.1 | uM | 0.272 | 0.335 | 0.611 | 0.357 | 0.239 | 0.424 | WV-14914 |
| 1 | uM | 0.431 | 0.365 | 0.351 | 0.296 | 0.269 | 0.166 | |
| 10 | uM | 0.387 | 0.304 | 0.516 | 0.049 | 0.043 | 0.020 | |
| 0.1 | uM | 0.380 | 0.503 | 0.509 | 0.369 | 0.451 | 0.417 | WV-15078 |
| 1 | uM | 0.629 | 0.344 | 0.612 | 0.335 | 0.224 | 0.220 | |
| 10 | uM | 0.378 | 0.280 | 0.363 | 0.010 | 0.028 | 0.011 | |
| 0.1 | uM | 0.407 | 0.366 | 0.538 | 0.466 | 0.537 | 0.398 | WV-15080 |
| 1 | uM | 0.348 | 0.383 | 0.624 | 0.270 | 0.244 | 0.364 | |
| 10 | uM | 0.327 | 0.239 | 0.198 | 0.026 | 0.117 | 0.002 | |

TABLE 41B

| | | wild type (G) | | | mutant (A) | | | |
|---|---|---|---|---|---|---|---|---|
| NTC 10 uM | | 0.314 | 0.435 | 0.325 | 0.629 | 0.776 | 0.521 | |
| 0.1 | uM | 0.392 | 0.356 | 0.288 | 0.480 | 0.480 | 0.451 | WV-12282 |
| 1 | uM | 0.403 | 0.402 | 0.381 | 0.444 | 0.414 | 0.427 | |
| 10 | uM | 0.729 | 0.198 | 0.456 | 0.312 | 0.225 | 0.243 | |
| 0.1 | uM | 0.187 | 0.357 | 0.214 | 0.318 | 0.542 | 0.440 | WV-12283 |
| 1 | uM | 0.433 | 0.349 | 0.353 | 0.595 | 0.484 | 0.445 | |
| 10 | uM | 0.263 | 0.180 | 0.325 | 0.329 | 0.388 | 0.277 | |
| 0.1 | uM | 0.266 | 0.228 | 0.470 | 0.363 | 0.345 | 0.565 | WV-14914 |
| 1 | uM | 0.434 | 0.344 | 0.217 | 0.294 | 0.291 | 0.301 | |
| 10 | uM | 0.295 | 0.202 | 0.380 | 0.141 | 0.145 | 0.156 | |
| 0.1 | uM | 0.309 | 0.413 | 0.389 | 0.441 | 0.542 | 0.537 | WV-15078 |
| 1 | uM | 0.615 | 0.317 | 0.493 | 0.350 | 0.251 | 0.339 | |
| 10 | uM | 0.241 | 0.170 | 0.227 | 0.147 | 0.139 | 0.147 | |
| 0.1 | uM | 0.358 | 0.414 | 0.417 | 0.516 | 0.489 | 0.519 | WV-15080 |
| 1 | uM | 0.331 | 0.336 | 0.587 | 0.288 | 0.291 | 0.402 | |
| 10 | uM | 0.148 | 0.150 | 0.068 | 0.205 | 0.207 | 0.132 | |

### Table 42. Activity of Certain Oligonucleotides.

Various HTT oligonucleotides were screened for their ability to knockdown mutant and wild-type HTT. Numbers indicate the % of HTT remaining (relative to control) at the indicated oligonucleotide concentrations. Data was normalized to controls; 100.0 would represent 100% wt or mutant HTT level (0% knockdown); and 0.0 would represent 0.0% HTT level (100.0% knockdown).

### Table 42A.

Neurons were derived from GM21756 patient-derived fibroblasts (heterozygous for the targeted SNP) and treated with 6.6 uM of the indicated oligonucleotide under gymnotic conditions for 7 days. RNA was quantified and normalized to control gene. Percentage of remaining wtHTT (wild-type HTT, WT) and mHTT (mutant HTT, or MU) mRNA is indicated. Negative control (PBS) and reference oligonucleotide WV-9679 were also tested (data not shown).

TABLE 42B

| | |
|---|---|
| WV-21405 | WV-21412 |

| WT | MU | WT | MU |
|---|---|---|---|
| 82 | 50 | 86 | 46 |

Neurons were derived from GM21756 patient-derived fibroblasts (heterozygous for the targeted SNP) and treated with 6.6 uM or 20 uM of the indicated oligonucleotide under gymnotic conditions for 7 days. RNA was quantified and normalized to TUBB3. Percentage of remaining wtHTT (wild-type HTT, WT) and mHTT (mutant HTT, or MU) mRNA is indicated. Negative control (PBS) and reference oligonucleotide WV-9679 were also tested (data not shown).

TABLE 43A
Activity of certain oligonucleotides.

| | |
|---|---|
| WV-21405 (6.6 uM) | WV-21412 (6.6 uM) |

| WT | MU | WT | MU |
|---|---|---|---|
| 80 | 46 | 90 | 41 |

| | |
|---|---|
| WV-21405 (20 uM) | WV-21412 (20 uM) |

| WT | MU | WT | MU |
|---|---|---|---|
| 82 | 50 | 86 | 46 |

### In Table 43A and 43B:

Various HTT oligonucleotides were tested for knockdown of HTT in vitro in neurons treated for 7 days. The concentration of oligonucleotide used is shown as exp 10 in uM. In this and various Tables, HTT RNA was quantified and normalized to TUBB3.
Numbers represent % of muHTT mRNA left after treatment with oligonucleotides. 100.0 would represent 100% HTT level (0% knockdown) and 0.0 would represent 0% HTT level (100% knock down).
Various oligonucleotides, including WV-14914 and those with an identical base sequence, target SNP rs362273, which aligns with position 10 of the sequence; the tested cells are homozygous for this SNP. In various Tables, the results of positive and negative controls performed may not all be shown. In this and various Tables, results of replicate experiments are shown. In this and various other Tables, Concentration (Conc.) of oligonucleotides are used. In this and various other Tables, ASO=oligonucleotide.

TABLE 43B
Activity of certain oligonucleotides.

| ASO | | | | | | | | |
|---|---|---|---|---|---|---|---|---|
| [uM] | WV-21403 | | | | WV-21404 | | | |
| 1.301 | 5.1 | 18.8 | 17.3 | 18.0 | 2.3 | 7.1 | 12.9 | |
| 0.824 | 8.9 | 30.6 | 27.0 | 31.3 | 7.4 | 18.3 | 8.6 | 21.1 |
| 0.347 | 11.7 | 72.4 | 51.3 | 77.3 | 23.3 | 51.2 | 32.8 | 53.5 |
| −0.130 | 84.5 | 90.0 | 85.5 | 70.5 | 46.6 | 67.3 | 57.6 | 50.6 |
| −0.607 | 70.7 | 98.1 | 85.9 | 103.4 | 67.3 | 110.4 | 64.0 | 78.0 |
| −1.085 | 82.1 | 99.2 | 100.1 | 87.5 | 87.6 | 101.7 | 104.0 | 85.7 |
| −1.562 | 78.1 | 70.8 | 118.7 | 91.8 | 62.6 | 79.7 | 129.8 | 101.1 |
| ASO | | | | | | | | |
| [uM] | WV-21406 | | | | WV-21405 | | | |
| 1.301 | 9.3 | 0.9 | 13.2 | 7.2 | 5.7 | 16.2 | 11.5 | 11.0 |
| 0.824 | 13.9 | 7.6 | 23.5 | 23.4 | 17.2 | 23.9 | 28.5 | 21.5 |
| 0.347 | 31.2 | 20.8 | 43.5 | 34.1 | 44.3 | 51.1 | 47.2 | 42.5 |
| −0.130 | 71.7 | 49.2 | 47.1 | 63.6 | 48.9 | 71.0 | 66.0 | 67.6 |
| −0.607 | 75.9 | 68.6 | 82.8 | 93.9 | 80.0 | 94.8 | 89.3 | 84.6 |
| −1.085 | 90.6 | 111.5 | 81.5 | 63.3 | 83.9 | 88.2 | 72.3 | 89.4 |
| −1.562 | 94.5 | 66.9 | 89.8 | 91.1 | 92.2 | 104.4 | 105.6 | 81.6 |
| ASO | | | | | | | | |
| [uM] | WV-21407 | | | | WV-21408 | | | |
| 1.301 | 55.8 | 65.7 | 95.7 | 69.0 | 13.6 | 22.2 | 20.4 | 10.9 |
| 0.824 | 77.0 | 105.1 | 82.9 | 66.8 | 28.0 | 44.2 | 30.4 | 33.2 |
| 0.347 | 69.9 | 131.3 | 96.7 | 115.2 | 47.6 | 55.5 | 58.1 | 57.3 |
| −0.130 | 83.5 | 128.3 | 79.8 | 90.3 | 93.1 | 84.6 | 91.8 | 69.6 |
| −0.607 | 94.1 | 87.8 | 89.9 | 111.7 | 74.3 | 84.4 | 113.8 | 86.0 |
| −1.085 | 106.2 | 109.0 | 98.1 | 89.8 | 91.0 | 114.7 | 101.2 | 81.5 |
| −1.562 | 114.1 | 106.5 | 95.8 | 107.8 | 114.7 | 124.5 | 111.7 | 88.5 |
| ASO | | | | | | | | |
| [uM] | WV-21409 | | | | WV-21410 | | | |
| 1.301 | 19.4 | 19.4 | 19.4 | 19.4 | 21.0 | 17.7 | 23.3 | 15.5 |
| 0.824 | 27.5 | 27.5 | 27.5 | 27.5 | 26.1 | 37.3 | 39.3 | 39.7 |
| 0.347 | 45.3 | 45.3 | 45.3 | 45.3 | 28.7 | 68.4 | 51.1 | 55.3 |
| −0.130 | 81.7 | 81.7 | 81.7 | 81.7 | 70.8 | 76.3 | 81.2 | 70.1 |
| −0.607 | 96.0 | 96.0 | 96.0 | 96.0 | 81.2 | 107.5 | 81.5 | 86.2 |
| −1.085 | 105.9 | 105.9 | 105.9 | 105.9 | 89.4 | 97.9 | 95.4 | 92.8 |
| −1.562 | 100.0 | 100.0 | 100.0 | 100.0 | 108.9 | 106.6 | 103.2 | 86.9 |
| ASO | | | | | | | | |
| [uM] | WV-14914 | | | | WV-9679 | | | |
| 1.301 | 9.4 | 28.3 | 13.1 | 18.9 | 21.5 | 42.9 | 33.0 | 40.1 |
| 0.824 | 18.6 | 27.4 | 26.9 | 27.3 | 40.6 | 66.1 | 60.0 | 69.2 |
| 0.347 | 41.2 | 40.9 | 37.3 | 37.1 | 55.0 | 90.4 | 96.1 | 61.0 |
| −0.130 | 53.5 | 76.2 | 64.3 | 34.6 | 87.9 | 66.9 | 75.0 | 81.5 |
| −0.607 | 100.8 | 83.1 | 97.3 | 75.3 | 93.9 | 114.7 | 111.3 | 91.1 |
| −1.085 | 117.0 | 90.6 | 81.5 | 85.9 | 128.8 | 109.0 | 94.6 | 96.3 |
| −1.562 | 95.6 | 117.4 | 119.1 | 108.4 | 109.1 | 108.0 | 75.7 | 65.6 |

TABLE 44
Activity of certain oligonucleotides.

| ASO | | | | | | | | |
|---|---|---|---|---|---|---|---|---|
| [uM] | WV-21411 | | | | WV-21412 | | | |
| 1.301 | 53.1 | 81.1 | 87.8 | 58.6 | 9.1 | 10.8 | 13.9 | 8.5 |
| 0.824 | 48.4 | 123.3 | 106.6 | 124.5 | 15.0 | 25.6 | 24.9 | 13.7 |
| 0.347 | 122.5 | 78.9 | 80.4 | 77.5 | 25.1 | 49.0 | 26.7 | 36.2 |
| −0.130 | 85.2 | 130.9 | 76.5 | 111.6 | 49.2 | 56.1 | 68.9 | 67.1 |
| −0.607 | 84.1 | 99.2 | 94.2 | 103.5 | 99.5 | 65.9 | 69.1 | 85.3 |
| −1.085 | 88.2 | 100.5 | 82.7 | 87.0 | 91.1 | 99.2 | 61.5 | 95.9 |
| −1.562 | 91.8 | 82.8 | 91.3 | 104.4 | 83.6 | 111.6 | 83.9 | 92.0 |

| | |
|---|---|
| ASO | |
| [uM] | WV-21447 |

| | | | | |
|---|---|---|---|---|
| 1.301 | 7.5 | 12.7 | 14.4 | 10.6 |
| 0.824 | 18.8 | 39.3 | 31.1 | 19.9 |
| 0.347 | 34.4 | 68.3 | 53.2 | 96.9 |
| −0.130 | 95.8 | 58.5 | 85.3 | 69.5 |
| −0.607 | 79.6 | 121.4 | 99.4 | 104.5 |
| −1.085 | 113.5 | 95.9 | 86.8 | 62.1 |
| −1.562 | 104.9 | 77.0 | 87.5 | 90.1 |

| ASO | | | | | | | | |
|---|---|---|---|---|---|---|---|---|
| [uM] | WV-21448 | | | | WV-14914 | | | |
| 1.301 | 48.8 | 68.1 | 84.3 | 69.9 | 7.6 | 15.7 | 11.0 | 17.6 |
| 0.824 | 67.3 | 107.7 | 64.3 | 86.2 | 12.1 | 32.6 | 18.6 | 24.2 |
| 0.347 | 75.2 | 112.9 | 97.9 | 79.5 | 23.7 | 33.9 | 28.5 | 40.8 |
| −0.130 | 89.3 | 102.4 | 90.9 | 109.6 | 62.1 | 93.1 | 75.2 | 79.4 |
| −0.607 | 98.7 | 121.0 | 85.8 | 112.7 | 85.2 | 88.2 | 72.6 | 89.2 |
| −1.085 | 93.5 | 86.1 | 86.4 | 105.5 | 79.5 | 95.3 | 77.4 | 76.2 |
| −1.562 | 83.6 | 105.7 | 81.6 | 81.6 | 115.7 | 90.2 | 88.5 | 84.6 |

This Table presents a summary of three independent experiments (n=1, 2 or 3) determining IC₅₀ in uM.

TABLE 45
Activity of certain oligonucleotides.

| | IC₅₀, uM | | |
|---|---|---|---|
| | n = 1 | n = 2 | n = 3 |
| WV-21403 | 2.31 | 2.8 | 1.66 |
| WV-21404 | 1.16 | 0.65 | 0.68 |
| WV-21405 | 1.23 | 0.38 | 0.75 |
| WV-21406 | 1.68 | 1.14 | 1.36 |
| WV-21408 | | 1.5 | |
| WV-21409 | 2.25 | ~2-3 | |
| WV-21410 | 2.06 | 1.07 | |
| WV-21412 | 1.12 | 0.63 | |
| WV-21447 | 3.29 | 1.8 | 1.99 |
| WV-21448 | 7.27 | | |
| WV-19824 | | 1.07 | 0.74 |
| WV-14914 | 0.6 | 0.95 | 1.05 |
| WV-9679 | 9.09 | 2.41 | 7.88 |

Various HTT oligonucleotides were tested for knockdown of HTT in neurons in vitro, with 7 day treatment. Neurons were heterozygous for the SNP targeted by various tested oligonucleotides. Numbers indicate the % of HTT remaining (relative to control) at the indicated oligonucleotide concentrations; knockdown of wild type HTT and mutant HTT are shown. 1.00 would represent 10000 HTT mRNA remaining (0.0% knockdown); and 0.0 will represent 0.00% HTT mRNA remaining (100.0% knockdown). NTC: Non-targeting control

TABLE 46
Activity of certain oligonucleotides.

| Oligonucleotide | Conc. | | wild type (G) | | | mutant (A) | | |
|---|---|---|---|---|---|---|---|---|
| NTC | 10 | uM | 0.406 | 0.430 | 0.544 | 0.509 | 0.494 | 0.617 |
| WV-14914 | 0.1 | uM | 0.278 | 0.202 | 0.273 | 0.339 | 0.243 | 0.358 |
| | 1 | uM | 0.277 | 0.386 | 0.268 | 0.176 | 0.173 | 0.119 |
| | 10 | uM | 0.303 | 0.144 | 0.260 | 0.032 | 0.022 | 0.010 |
| WV-21404 | 0.1 | uM | 0.208 | 0.184 | 0.267 | 0.192 | 0.213 | 0.274 |
| | 1 | uM | 0.219 | 0.200 | 0.201 | 0.101 | 0.100 | 0.132 |
| | 10 | uM | 0.265 | 0.146 | 0.225 | 0.014 | 0.033 | 0.024 |
| WV-21405 | 0.1 | uM | 0.317 | 0.224 | 0.209 | 0.267 | 0.314 | 0.238 |
| | 1 | uM | 0.308 | 0.315 | 0.225 | 0.149 | 0.134 | 0.105 |
| | 10 | uM | 0.265 | 0.159 | 0.231 | 0.034 | 0.031 | 0.015 |
| WV-21406 | 0.1 | uM | 0.344 | 0.117 | 0.199 | 0.308 | 0.171 | 0.218 |
| | 1 | uM | 0.137 | 0.237 | 0.357 | 0.095 | 0.148 | 0.186 |
| | 10 | uM | 0.246 | 0.151 | 0.290 | 0.025 | 0.027 | 0.018 |
| WV-21412 | 0.1 | uM | 0.242 | 0.237 | 0.211 | 0.194 | 0.217 | 0.200 |
| | 1 | uM | 0.202 | 0.327 | 0.148 | 0.109 | 0.132 | 0.107 |
| | 10 | uM | 0.285 | 0.164 | 0.222 | 0.010 | 0.028 | 0.014 |

Various HTT oligonucleotides were tested for knockdown of HTT in GM21756-2 NPCs in vitro at indicated concentrations. Experiment involved 5 day treatment.
In this and various other Tables, characteristics of cells used are as follows:

| | CAG | rs362307 | rs362331 | rs362273 | rs363099 | rs362272 |
|---|---|---|---|---|---|---|
| Line | repeats | (C/T) | (C/T) | (G/A) | (T/C) | (A/G) |
| ND40536 | 66/? | C/T | T/T | A/A | C/C | G/G |
| GM21756 | 69/15 | C/C | C/T | G/A | T/C | A/G |
| (P6) | | | | | | |

Numbers indicate the % of HTT remaining (relative to control) at the indicated oligonucleotide concentrations, normalized to NTC. 1.00 would represent 100% HTT mRNA remaining (0.0% knockdown); and 0.0 will represent 0.00% HTT mRNA remaining (100.0% knockdown); knockdown of wild type HTT and mutant HTT are shown. WV-12890 is a non-targeting control (NTC).

TABLE 47
Activity of certain oligonucleotides.

| | | | wild type (G) | | | mutant (A) | | |
|---|---|---|---|---|---|---|---|---|
| water | | | 0.869 | 1.073 | 1.057 | 0.960 | 1.072 | 0.969 |
| WV-12890 | 0.1 | uM | 0.988 | 1.128 | 1.097 | 1.212 | 1.240 | 1.235 |
| | 1 | uM | 1.029 | 1.138 | 0.962 | 1.178 | 1.102 | 1.045 |
| | 10 | uM | 1.001 | 0.802 | 1.005 | 1.186 | 1.092 | 1.206 |
| WV-14914 | 0.1 | uM | 0.863 | 0.903 | 0.952 | 1.074 | 0.894 | 0.788 |
| | 1 | uM | 1.009 | 0.969 | 1.019 | 1.021 | 0.878 | 0.920 |
| | 10 | uM | 0.978 | 1.069 | 1.021 | 0.516 | 0.562 | 0.613 |
| WV-22937 | 0.1 | uM | 1.088 | 1.064 | 1.266 | 1.224 | 1.264 | 0.861 |
| | 1 | uM | 1.129 | 1.001 | 0.885 | 1.160 | 1.205 | 0.960 |
| | 10 | uM | 1.329 | 0.908 | 1.268 | 1.489 | 1.015 | 1.561 |
| WV-22955 | 0.1 | uM | 0.933 | 1.003 | 1.125 | 1.024 | 0.961 | 1.197 |
| | 1 | uM | 0.883 | 0.873 | 0.778 | 0.858 | 0.990 | 0.825 |
| | 10 | uM | 0.974 | 0.981 | 0.981 | 0.968 | 1.056 | 0.899 |
| WV-9679 | 0.1 | uM | 0.976 | 1.031 | 1.084 | 1.065 | 1.094 | 1.174 |
| | 1 | uM | 0.829 | 0.820 | 0.941 | 0.877 | 0.891 | 0.989 |
| | 10 | uM | 0.621 | 0.657 | 0.655 | 0.519 | 0.688 | 0.618 |

Various HTT oligonucleotides, including pan-specific HTT oligonucleotides, were tested for knockdown of HTT in wt mouse neurons in vitro at a concentration of 10 uM.
Numbers indicate the % of HTT remaining (relative to control). 1.00 would represent 100% HTT mRNA remaining (0.0% knockdown), and 0.0 will represent 0.0% HTT mRNA remaining (100.0% q knockdown).

TABLE 48
Activity of certain oligonucleotides.

| WV- | WV- | WV- | WV- | WV- | WV- | WV- | WV- | WV- |
|---|---|---|---|---|---|---|---|---|
| 21182 | 21183 | 21184 | 21185 | 21186 | 21187 | 21188 | 21189 | 21190 |
| 0.307 | 0.054 | 0.014 | 0.065 | 0.075 | 0.341 | 0.408 | 0.137 | 0.729 |
| 0.27 | 0.142 | 0.018 | 0.148 | 0.076 | 0.505 | 0.252 | 0.167 | 0.694 |
| WV- | WV- | WV- | WV- | WV- | WV- | WV- | WV- | WV- |
| 21191 | 21192 | 21193 | 21194 | 21195 | 21196 | 21197 | 21198 | 21199 |
| 0.382 | 0.494 | 0.607 | 0.999 | 0.553 | 0.759 | 0.617 | 0.696 | 0.313 |
| 0.588 | 0.721 | 1.1 | 0.918 | 0.675 | 0.882 | 0.589 | 0.682 | 0.434 |

| WV- | WV- | WV- | WV- | WV- | WV- | WV-8587 | WV-9491 |
|---|---|---|---|---|---|---|---|
| 21200 | 21201 | 21202 | 21203 | 21204 | 21205 | NTC | NTC |
| 0.375 | 0.493 | 0.234 | 0.665 | 0.469 | 0.641 | 1.223 | 0.85 |
| 0.467 | 0.524 | 0.454 | 0.492 | 0.497 | 0.739 | 0.837 | 1.076 |

Various HTT oligonucleotides were tested for knockdown of HTT in GM21756 patient-derived neurons in vitro. Experiment involved 30 day differentiation, and 7 day treatment. The cells tested were heterozygous for the SNP targeted by the oligonucleotides.
Numbers indicate the % of HTT remaining (relative to control) at the indicated oligonucleotide concentrations. 1.00 would represent 100% HTT mRNA remaining (0.0% knockdown); and 0.0 will represent 0.00% HTT mRNA remaining (100.0% knockdown); knockdown of wild type HTT and mutant HTT are shown.

TABLE 49
Activity of certain oligonucleotides.

| Oligonucleotide | Conc. | | wild type (G) | | | mutant (A) | | |
|---|---|---|---|---|---|---|---|---|
| WV-9679 | 0.009 | uM | 0.560 | 0.529 | 0.503 | 0.368 | 0.370 | 0.365 |
| | 0.027 | uM | 0.499 | 0.463 | 0.444 | 0.374 | 0.365 | 0.320 |
| | 0.082 | uM | 0.480 | 0.485 | 0.481 | 0.349 | 0.319 | 0.355 |
| | 0.246 | uM | 0.394 | 0.369 | 0.464 | 0.275 | 0.291 | 0.346 |
| | 0.740 | uM | 0.426 | 0.413 | 0.393 | 0.293 | 0.264 | 0.340 |
| | 2.222 | uM | 0.364 | 0.376 | 0.384 | 0.268 | 0.269 | 0.274 |
| | 6.666 | uM | 0.297 | 0.338 | 0.293 | 0.251 | 0.196 | 0.257 |
| | 20 | uM | 0.266 | 0.237 | 0.203 | 0.238 | 0.202 | 0.154 |
| WV-14914 | 0.009 | uM | 0.576 | 0.598 | 0.616 | 0.400 | 0.403 | 0.413 |
| | 0.027 | uM | 0.548 | 0.532 | 0.476 | 0.395 | 0.339 | 0.339 |
| | 0.082 | uM | 0.537 | 0.587 | 0.548 | 0.341 | 0.386 | 0.367 |
| | 0.246 | uM | 0.541 | 0.550 | 0.490 | 0.354 | 0.252 | 0.314 |
| | 0.740 | uM | 0.508 | 0.511 | 0.547 | 0.324 | 0.304 | 0.266 |
| | 2.222 | uM | 0.486 | 0.478 | 0.396 | 0.286 | 0.262 | 0.131 |
| | 6.666 | uM | 0.628 | 0.499 | 0.593 | 0.180 | 0.220 | 0.248 |
| | 20 | uM | 0.573 | 0.538 | 0.656 | 0.236 | 0.234 | 0.255 |
| WV-21404 | 0.009 | uM | 0.508 | 0.599 | 0.500 | 0.435 | 0.337 | 0.317 |
| | 0.027 | uM | 0.562 | 0.536 | 0.595 | 0.358 | 0.332 | 0.365 |
| | 0.082 | uM | 0.539 | 0.587 | 0.512 | 0.297 | 0.337 | 0.347 |
| | 0.246 | uM | 0.528 | 0.563 | 0.479 | 0.293 | 0.303 | 0.295 |
| | 0.740 | uM | 0.481 | 0.555 | 0.557 | 0.218 | 0.272 | 0.285 |
| | 2.222 | uM | 0.555 | 0.573 | 0.540 | 0.232 | 0.246 | 0.224 |
| | 6.666 | uM | 0.580 | 0.518 | 0.544 | 0.221 | 0.221 | 0.218 |
| | 20 | uM | 0.578 | 0.586 | 0.528 | 0.193 | 0.216 | 0.176 |
| WV-21405 | 0.009 | uM | 0.551 | 0.637 | 0.516 | 0.368 | 0.377 | 0.312 |
| | 0.027 | uM | 0.505 | 0.494 | 0.478 | 0.332 | 0.354 | 0.299 |
| | 0.082 | uM | 0.458 | 0.621 | 0.538 | 0.345 | 0.379 | 0.330 |
| | 0.246 | uM | 0.449 | 0.511 | 0.554 | 0.291 | 0.304 | 0.353 |
| | 0.740 | uM | 0.463 | 0.471 | 0.543 | 0.277 | 0.258 | 0.280 |
| | 2.222 | uM | 0.517 | 0.462 | 0.450 | 0.224 | 0.215 | 0.224 |
| | 6.666 | uM | 0.478 | 0.500 | 0.536 | 0.218 | 0.198 | 0.229 |
| | 20 | uM | 0.469 | 0.493 | 0.518 | 0.185 | 0.176 | 0.225 |
| WV-21406 | 0.009 | uM | 0.488 | 0.657 | 0.665 | 0.310 | 0.358 | 0.416 |
| | 0.027 | uM | 0.437 | 0.563 | 0.564 | 0.276 | 0.337 | 0.329 |
| | 0.082 | uM | 0.501 | 0.508 | 0.566 | 0.337 | 0.379 | 0.370 |
| | 0.246 | uM | 0.450 | 0.503 | 0.482 | 0.295 | 0.324 | 0.317 |
| | 0.740 | uM | 0.436 | 0.474 | 0.515 | 0.282 | 0.275 | 0.270 |
| | 2.222 | uM | 0.421 | 0.498 | 0.443 | 0.257 | 0.252 | 0.238 |
| | 6.666 | uM | 0.490 | 0.564 | 0.536 | 0.218 | 0.280 | 0.209 |
| | 20 | uM | 0.507 | 0.670 | 0.704 | 0.221 | 0.169 | 0.146 |
| WV-21412 | 0.009 | uM | 0.569 | 0.629 | 0.650 | 0.397 | 0.418 | 0.371 |
| | 0.027 | uM | 0.499 | 0.529 | 0.531 | 0.303 | 0.310 | 0.342 |
| | 0.082 | uM | 0.657 | 0.486 | 0.549 | 0.354 | 0.324 | 0.338 |
| | 0.246 | uM | 0.634 | 0.527 | 0.497 | 0.323 | 0.300 | 0.283 |
| | 0.740 | uM | 0.566 | 0.538 | 0.510 | 0.275 | 0.277 | 0.279 |
| | 2.222 | uM | 0.562 | 0.544 | 0.513 | 0.242 | 0.281 | 0.240 |
| | 6.666 | uM | 0.561 | 0.520 | 0.513 | 0.192 | 0.218 | 0.185 |
| | 20 | uM | 0.622 | 0.530 | 0.512 | 0.094 | 0.228 | 0.206 |
| WV-12892 | 0.009 | uM | 0.544 | 0.652 | 0.653 | 0.418 | 0.435 | 0.425 |
| | 0.027 | uM | 0.514 | 0.501 | 0.572 | 0.375 | 0.401 | 0.391 |
| | 0.082 | uM | 0.548 | 0.527 | 0.537 | 0.421 | 0.365 | 0.343 |
| | 0.246 | uM | 0.517 | 0.471 | 0.664 | 0.382 | 0.407 | 0.475 |
| | 0.740 | uM | 0.499 | 0.563 | 0.490 | 0.374 | 0.335 | 0.347 |
| | 2.222 | uM | 0.466 | 0.507 | 0.428 | 0.399 | 0.376 | 0.362 |
| | 6.666 | uM | 0.477 | 0.502 | 0.484 | 0.301 | 0.314 | 0.399 |
| | 20 | uM | 0.557 | 0.510 | 0.518 | 0.361 | 0.405 | 0.386 |
| PBS | | | 0.544 | 0.652 | 0.653 | 0.418 | 0.435 | 0.425 |

Various HTT oligonucleotides were tested for knockdown of HTT in GM21756-2 cells in vitro with 30 day differentiation and 7 day treatment.
Numbers indicate the % of HTT remaining (relative to control) at the indicated oligonucleotide concentrations. 1.00 would represent 100% HTT mRNA remaining (0.0% knockdown); and 0.0 will represent 0.00% HTT mRNA remaining (100.0% knockdown); knockdown of wild type HTT and mutant HTT are shown.

TABLE 50
Activity of certain oligonucleotides.

| Oligonucleotide | Conc. | | wild type (G) | | | mutant (A) | | |
|---|---|---|---|---|---|---|---|---|
| WV-9679 | 0.009 | uM | 0.560 | 0.529 | 0.503 | 0.368 | 0.370 | 0.365 |
| | 0.027 | uM | 0.499 | 0.463 | 0.444 | 0.374 | 0.365 | 0.320 |
| | 0.082 | uM | 0.480 | 0.485 | 0.481 | 0.349 | 0.319 | 0.355 |
| | 0.246 | uM | 0.394 | 0.369 | 0.464 | 0.275 | 0.291 | 0.346 |
| | 0.740 | uM | 0.426 | 0.413 | 0.393 | 0.293 | 0.264 | 0.340 |
| | 2.222 | uM | 0.364 | 0.376 | 0.384 | 0.268 | 0.269 | 0.274 |
| | 6.666 | uM | 0.297 | 0.338 | 0.293 | 0.251 | 0.196 | 0.257 |
| | 20 | uM | 0.266 | 0.237 | 0.203 | 0.238 | 0.202 | 0.154 |
| WV-14914 | 0.009 | uM | 0.576 | 0.598 | 0.616 | 0.400 | 0.403 | 0.413 |
| | 0.027 | uM | 0.548 | 0.532 | 0.476 | 0.395 | 0.339 | 0.339 |
| | 0.082 | uM | 0.537 | 0.587 | 0.548 | 0.341 | 0.386 | 0.367 |
| | 0.246 | uM | 0.541 | 0.550 | 0.490 | 0.354 | 0.252 | 0.314 |
| | 0.740 | uM | 0.508 | 0.511 | 0.547 | 0.324 | 0.304 | 0.266 |
| | 2.222 | uM | 0.486 | 0.478 | 0.396 | 0.286 | 0.262 | 0.131 |
| | 6.666 | uM | 0.628 | 0.499 | 0.593 | 0.180 | 0.220 | 0.248 |
| | 20 | uM | 0.573 | 0.538 | 0.656 | 0.236 | 0.234 | 0.255 |
| WV-21404 | 0.009 | uM | 0.508 | 0.599 | 0.500 | 0.435 | 0.337 | 0.317 |
| | 0.027 | uM | 0.562 | 0.536 | 0.595 | 0.358 | 0.332 | 0.365 |
| | 0.082 | uM | 0.539 | 0.587 | 0.512 | 0.297 | 0.337 | 0.347 |
| | 0.246 | uM | 0.528 | 0.563 | 0.479 | 0.293 | 0.303 | 0.295 |
| | 0.740 | uM | 0.481 | 0.555 | 0.557 | 0.218 | 0.272 | 0.285 |
| | 2.222 | uM | 0.555 | 0.573 | 0.540 | 0.232 | 0.246 | 0.224 |
| | 6.666 | uM | 0.580 | 0.518 | 0.544 | 0.221 | 0.221 | 0.218 |
| | 20 | uM | 0.578 | 0.586 | 0.528 | 0.193 | 0.216 | 0.176 |
| WV-21405 | 0.009 | uM | 0.551 | 0.637 | 0.516 | 0.368 | 0.377 | 0.312 |
| | 0.027 | uM | 0.505 | 0.494 | 0.478 | 0.332 | 0.354 | 0.299 |
| | 0.082 | uM | 0.458 | 0.621 | 0.538 | 0.345 | 0.379 | 0.330 |
| | 0.246 | uM | 0.449 | 0.511 | 0.554 | 0.291 | 0.304 | 0.353 |
| | 0.740 | uM | 0.463 | 0.471 | 0.543 | 0.277 | 0.258 | 0.280 |
| | 2.222 | uM | 0.517 | 0.462 | 0.450 | 0.224 | 0.215 | 0.224 |
| | 6.666 | uM | 0.478 | 0.500 | 0.536 | 0.218 | 0.198 | 0.229 |
| | 20 | uM | 0.469 | 0.493 | 0.518 | 0.185 | 0.176 | 0.225 |
| WV-21406 | 0.009 | uM | 0.488 | 0.657 | 0.665 | 0.310 | 0.358 | 0.416 |
| | 0.027 | uM | 0.437 | 0.563 | 0.564 | 0.276 | 0.337 | 0.329 |
| | 0.082 | uM | 0.501 | 0.508 | 0.566 | 0.337 | 0.379 | 0.370 |
| | 0.246 | uM | 0.450 | 0.503 | 0.482 | 0.295 | 0.324 | 0.317 |
| | 0.740 | uM | 0.436 | 0.474 | 0.515 | 0.282 | 0.275 | 0.270 |
| | 2.222 | uM | 0.421 | 0.498 | 0.443 | 0.257 | 0.252 | 0.238 |
| | 6.666 | uM | 0.490 | 0.564 | 0.536 | 0.218 | 0.280 | 0.209 |
| | 20 | uM | 0.507 | 0.670 | 0.704 | 0.221 | 0.169 | 0.146 |
| WV-21412 | 0.009 | uM | 0.569 | 0.629 | 0.650 | 0.397 | 0.418 | 0.371 |
| | 0.027 | uM | 0.499 | 0.529 | 0.531 | 0.303 | 0.310 | 0.342 |
| | 0.082 | uM | 0.657 | 0.486 | 0.549 | 0.354 | 0.324 | 0.338 |
| | 0.246 | uM | 0.634 | 0.527 | 0.497 | 0.323 | 0.300 | 0.283 |
| | 0.740 | uM | 0.566 | 0.538 | 0.510 | 0.275 | 0.277 | 0.279 |
| | 2.222 | uM | 0.562 | 0.544 | 0.513 | 0.242 | 0.281 | 0.240 |
| | 6.666 | uM | 0.561 | 0.520 | 0.513 | 0.192 | 0.218 | 0.185 |
| | 20 | uM | 0.622 | 0.530 | 0.512 | 0.094 | 0.228 | 0.206 |
| WV-12892 | 0.009 | uM | 0.544 | 0.652 | 0.653 | 0.418 | 0.435 | 0.425 |
| | 0.027 | uM | 0.514 | 0.501 | 0.572 | 0.375 | 0.401 | 0.391 |
| | 0.082 | uM | 0.548 | 0.527 | 0.537 | 0.421 | 0.365 | 0.343 |
| | 0.246 | uM | 0.517 | 0.471 | 0.664 | 0.382 | 0.407 | 0.475 |
| | 0.740 | uM | 0.499 | 0.563 | 0.490 | 0.374 | 0.335 | 0.347 |
| | 2.222 | uM | 0.466 | 0.507 | 0.428 | 0.399 | 0.376 | 0.362 |
| | 6.666 | uM | 0.477 | 0.502 | 0.484 | 0.301 | 0.314 | 0.399 |
| | 20 | uM | 0.557 | 0.510 | 0.518 | 0.361 | 0.405 | 0.386 |
| PBS | | | 0.544328 | 0.652475 | 0.653357 | 0.418487 | 0.43539 | 0.425176 |

Various HTT oligonucleotides were tested for knockdown of HTT in iNeurons in vitro.
The concentration of oligonucleotide used is shown as exp 10 in uM ([uM]).
Numbers represent % of HTT mRNA left after treatment with oligonucleotides. 100.0 would represent 100% HTT level (0% knockdown) and 0.0 would represent 0% HTT level (100% knock down). In this and various tables, ASO=oligonucleotide.

TABLE 51A
Activity of certain oligonucleotides.

| ASO | | | | | | | | |
|---|---|---|---|---|---|---|---|---|
| [uM] | WV-21404 | | | | WV-21405 | | | |
| 1.301 | 2.3 | 7.1 | 12.9 | | 0.9 | 13.2 | 7.2 | 5.7 |
| 0.824 | 7.4 | 18.3 | 8.6 | 21.1 | 7.6 | 23.5 | 23.4 | 17.2 |
| 0.347 | 23.3 | 51.2 | 32.8 | 53.5 | 20.8 | 43.5 | 34.1 | 44.3 |
| −0.130 | 46.6 | 67.3 | 57.6 | 50.6 | 49.2 | 47.1 | 63.6 | 48.9 |
| −0.607 | 67.3 | 110.4 | 64.0 | 78.0 | 68.6 | 82.8 | 93.9 | 80.0 |
| −1.085 | 87.6 | 101.7 | 104.0 | 85.7 | 111.5 | 81.5 | 63.3 | 83.9 |
| −1.562 | 62.6 | 79.7 | 129.8 | 101.1 | 66.9 | 89.8 | 91.1 | 92.2 |
| ASO | | | | | | | | |
| [uM] | WV-21406 | | | | WV-21412 | | | |
| 1.301 | 9.3 | 16.2 | 11.5 | 11.0 | 9.1 | 10.8 | 13.9 | 8.5 |
| 0.824 | 13.9 | 23.9 | 28.5 | 21.5 | 15.0 | 25.6 | 24.9 | 13.7 |
| 0.347 | 31.2 | 51.1 | 47.2 | 42.5 | 25.1 | 49.0 | 26.7 | 36.2 |
| −0.130 | 71.7 | 71.0 | 66.0 | 67.6 | 49.2 | 56.1 | 68.9 | 67.1 |
| −0.607 | 75.9 | 94.8 | 89.3 | 84.6 | 99.5 | 65.9 | 69.1 | 85.3 |
| −1.085 | 90.6 | 88.2 | 72.3 | 89.4 | 91.1 | 99.2 | 61.5 | 95.9 |
| −1.562 | 94.5 | 104.4 | 105.6 | 81.6 | 83.6 | 111.6 | 83.9 | 92.0 |
| ASO | | | | | | | | |
| [uM] | WV-14914 | | | | WV-9679 | | | |
| 1.301 | 7.6 | 15.7 | 11.0 | 17.6 | 21.5 | 42.9 | 33.0 | 40.1 |
| 0.824 | 12.1 | 32.6 | 18.6 | 24.2 | 40.6 | 66.1 | 60.0 | 69.2 |
| 0.347 | 23.7 | 33.9 | 28.5 | 40.8 | 55.0 | 90.4 | 96.1 | 61.0 |
| −0.130 | 62.1 | 93.1 | 75.2 | 79.4 | 87.9 | 66.9 | 75.0 | 81.5 |
| −0.607 | 85.2 | 88.2 | 72.6 | 89.2 | 93.9 | 114.7 | 111.3 | 91.1 |
| −1.085 | 79.5 | 95.3 | 77.4 | 76.2 | 128.8 | 109.0 | 94.6 | 96.3 |
| −1.562 | 115.7 | 90.2 | 88.5 | 84.6 | 109.1 | 108.0 | 75.7 | 65.6 |

### In Tables 51A and 51B:

Various HTT oligonucleotides were tested for knockdown of HTT in GM21756-2 cells in vitro with 7 day treatment. In this and various other Tables, experiment involved 2 weeks of differentiation from NPCs (neural progenitor cells) prior to treatment with oligonucleotide.
Numbers indicate the % of HTT remaining (relative to control) at the indicated oligonucleotide concentrations. 1.00 would represent 100% HTT mRNA remaining (0.0% knockdown); and 0.0 will represent 0.00% HTT mRNA remaining (100.0% knockdown); knockdown of wild type HTT and mutant HTT are shown. In this and various Tables, WV-9679 and other oligonucleotides with an identical or overlapping base sequence are pan-specific.

TABLE 51B
Activity of certain oligonucleotides.

| | | | wild type (G) | | | mutant (A) | | |
|---|---|---|---|---|---|---|---|---|
| PBS | | | 0.465 | 0.487 | 0.538 | 0.466 | 0.524 | 0.519 |
| WV-12892 (NTC) | 1 | uM | 0.436 | 0.361 | 0.395 | 0.504 | 0.503 | 0.474 |
| | 3 | uM | 0.396 | 0.366 | 0.378 | 0.476 | 0.525 | 0.450 |
| | 10 | uM | 0.406 | 0.440 | 0.399 | 0.416 | 0.406 | 0.436 |
| | 25 | uM | 0.440 | 0.435 | 0.423 | 0.399 | 0.462 | 0.437 |
| | 75 | uM | 0.455 | 0.569 | 0.597 | 0.571 | 0.653 | 0.625 |
| WV-14914 | 1 | uM | 0.410 | 0.450 | 0.510 | 0.419 | 0.496 | 0.484 |
| | 3 | uM | 0.385 | 0.487 | 0.529 | 0.327 | 0.497 | 0.361 |
| | 10 | uM | 0.465 | 0.531 | 0.557 | 0.242 | 0.347 | 0.314 |
| | 25 | uM | 0.414 | 0.526 | 0.656 | 0.155 | 0.338 | 0.205 |
| | 75 | uM | 0.288 | 0.721 | 0.741 | 0.063 | 0.135 | 0.113 |
| WV-9679 | 1 | uM | 0.384 | 0.428 | 0.436 | 0.512 | 0.441 | 0.434 |
| | 3 | uM | 0.359 | 0.383 | 0.427 | 0.544 | 0.420 | 0.491 |
| | 10 | uM | 0.283 | 0.344 | 0.387 | 0.440 | 0.346 | 0.346 |
| | 25 | uM | 0.303 | 0.317 | 0.290 | 0.261 | 0.311 | 0.289 |
| | 75 | uM | 0.171 | 0.274 | 0.189 | 0.206 | 0.235 | 0.180 |

TABLE 52
Activity of certain oligonucleotides.

| Oligonucleotide | Conc. | | wild type (G) | | | mutant (A) | | |
|---|---|---|---|---|---|---|---|---|
| PBS | | | 0.936 | 0.981 | 1.083 | 0.927 | 1.042 | 1.031 |
| WV-12892 | 1 | uM | 0.877 | 0.726 | 0.796 | 1.002 | 0.999 | 0.942 |
| | 3 | uM | 0.798 | 0.737 | 0.762 | 0.946 | 1.044 | 0.894 |
| | 10 | uM | 0.818 | 0.885 | 0.802 | 0.827 | 0.807 | 0.866 |
| | 25 | uM | 0.885 | 0.876 | 0.852 | 0.793 | 0.918 | 0.868 |
| | 75 | uM | 0.915 | 1.144 | 1.201 | 1.135 | 1.298 | 1.242 |
| WV-14914 | 1 | uM | 0.826 | 0.907 | 1.027 | 0.833 | 0.986 | 0.963 |
| | 3 | uM | 0.776 | 0.980 | 1.065 | 0.649 | 0.987 | 0.718 |
| | 10 | uM | 0.935 | 1.068 | 1.120 | 0.480 | 0.689 | 0.624 |
| | 25 | uM | 0.834 | 1.058 | 1.320 | 0.309 | 0.671 | 0.408 |
| | 75 | uM | 0.580 | 1.452 | 1.491 | 0.125 | 0.268 | 0.225 |
| WV-9679 | 1 | uM | 0.773 | 0.861 | 0.877 | 1.019 | 0.877 | 0.863 |
| | 3 | uM | 0.722 | 0.771 | 0.859 | 1.082 | 0.834 | 0.976 |
| | 10 | uM | 0.570 | 0.692 | 0.778 | 0.875 | 0.688 | 0.688 |
| | 25 | uM | 0.610 | 0.639 | 0.583 | 0.519 | 0.617 | 0.575 |
| | 75 | uM | 0.345 | 0.551 | 0.381 | 0.410 | 0.467 | 0.357 |

Various HTT oligonucleotides were tested for knockdown of HTT in ND40536 cells in vitro.
The concentration of oligonucleotide used is shown as exp 10 in uM (log). Cells tested were homozygous for the SNP targeted by the oligonucleotides.
Numbers indicate the % of HTT remaining (relative to control) at the indicated oligonucleotide concentrations. 1.00 would represent 100% HTT mRNA remaining (0.0% knockdown); and 0.0 will represent 0.00% HTT mRNA remaining (100.0% knockdown).

TABLE 53
Activity of certain oligonucleotides.

| log | WV-9679 | | | WV-14914 | | |
|---|---|---|---|---|---|---|
| −2.000 | 0.986 | 1.019 | 0.979 | 0.972 | 0.994 | 1.005 |
| −1.569 | 0.862 | 0.961 | 0.851 | 0.958 | 0.887 | 0.846 |
| −1.097 | 0.938 | 0.870 | 0.908 | 0.962 | 0.958 | 0.889 |
| −0.620 | 0.856 | 0.767 | 0.820 | 0.809 | 0.892 | 0.769 |
| −0.131 | 0.749 | 0.740 | 0.685 | 0.681 | 0.682 | 0.630 |
| 0.346 | 0.528 | 0.451 | 0.521 | 0.342 | 0.373 | 0.339 |
| 0.823 | 0.265 | 0.256 | 0.259 | 0.170 | 0.157 | 0.158 |
| 1.301 | 0.097 | 0.113 | 0.103 | 0.085 | 0.117 | 0.096 |

| log | WV-21404 | | | WV-21405 | | | WV-21406 | | |
|---|---|---|---|---|---|---|---|---|---|
| −2.000 | 1.210 | 1.066 | 1.087 | 0.937 | 1.182 | 1.116 | 0.964 | 1.047 | 1.186 |
| −1.569 | 1.150 | 1.029 | 0.838 | 1.009 | 0.970 | 1.003 | 1.008 | 0.904 | 0.919 |
| −1.097 | 1.036 | 0.997 | 1.060 | 0.926 | 0.961 | 0.978 | 1.068 | 1.038 | 1.194 |
| −0.620 | 0.792 | 0.625 | 0.787 | 0.627 | 0.856 | 0.759 | 0.876 | 0.949 | 0.838 |
| −0.131 | 0.621 | 0.595 | 0.753 | 0.594 | 0.551 | 0.638 | 0.661 | 0.686 | 0.680 |
| 0.346 | 0.369 | 0.348 | 0.341 | 0.402 | 0.393 | 0.361 | 0.459 | 0.454 | 0.482 |
| 0.823 | 0.139 | 0.108 | 0.108 | 0.186 | 0.181 | 0.161 | 0.196 | 0.224 | 0.230 |
| 1.301 | 0.027 | 0.035 | 0.034 | 0.059 | 0.043 | 0.045 | 0.076 | 0.064 | 0.070 |
| log | WV-21412 | | | WV-12892 | | | PBS | | |
| −2.000 | 1.010 | 0.993 | 0.998 | | | | 1.069 | 0.904 | 1.034 |
| −1.569 | 0.904 | 0.996 | 1.071 | 0.829 | 0.842 | 0.831 | 1.186 | 0.843 | |
| −1.097 | 0.869 | 0.938 | 0.949 | 1.077 | 0.921 | 0.970 | | | |
| −0.620 | 0.781 | 0.783 | 0.860 | 1.066 | 0.970 | 0.935 | | | |
| −0.131 | 0.544 | 0.573 | 0.556 | 0.903 | 0.936 | 0.874 | | | |
| 0.346 | 0.280 | 0.245 | 0.287 | 0.990 | 0.922 | 0.959 | | | |
| 0.823 | 0.104 | 0.089 | 0.083 | 0.942 | 0.978 | 0.969 | | | |
| 1.301 | 0.035 | 0.028 | 0.031 | 0.973 | 0.916 | 0.856 | | | |

Various HTT oligonucleotides, including various pan-specific HTT oligonucleotides, were tested for knockdown of HTT in human iCell neurons in vitro.
In Table 53 and various Table 54 tables, the concentrations of oligonucleotide used are shown in uM. Numbers represent % of HTT mRNA left after treatment with oligonucleotides. 100.0 would represent 100% HTT level (0% knockdown) and 0.0 would represent 0% HTT level (100% knock down).

TABLE 54A
Activity of certain oligonucleotides.

| | | |
|---|---|---|
| Conc. | WV-10787 | WV-10790 |

| | | | | | | | | |
|---|---|---|---|---|---|---|---|---|
| 10 | 13.7 | 17.3 | 29.0 | 36.8 | 39.0 | 43.1 | 40.2 | 58.5 |
| 3 | 17.4 | 44.1 | 71.6 | 49.8 | 33.8 | 43.9 | 46.1 | 49.8 |
| 1 | 69.0 | 67.2 | 86.8 | 68.6 | 47.7 | 60.9 | | 61.8 |

| Conc. | WV-21178 | | | | WV-21179 | | | | WV-9679 | | | |
|---|---|---|---|---|---|---|---|---|---|---|---|---|
| 10 | 17.9 | 12.9 | 16.8 | 15.2 | 33.7 | 28.9 | 25.3 | 32.2 | 15.7 | | 14.8 | 21.6 |
| 3 | 21.5 | 28.3 | 22.4 | 34.6 | 17.4 | 45.8 | 40.8 | 57.2 | 39.8 | 37.6 | 41.0 | 25.2 |
| 1 | 27.9 | 56.5 | 50.4 | 36.4 | 97.0 | | 72.0 | 80.9 | 43.6 | | 87.4 | 68.7 |
| Conc. | WV-21180 | | | | WV-21181 | | | | WV-9679 | | | |
| 10 | 24.1 | 22.2 | 53.8 | 40.3 | | 16.3 | 30.8 | 33.9 | 17.3 | 26.2 | 23.3 | 21.3 |
| 3 | 40.2 | 46.2 | 53.3 | 49.6 | 36.5 | 30.3 | 52.2 | 34.7 | 33.0 | 29.9 | 35.5 | 33.6 |
| 1 | 79.9 | 58.9 | 84.0 | 80.7 | 48.2 | 46.3 | 57.7 | 62.5 | 53.2 | 51.3 | 55.6 | 41.3 |

In Table 54A, B and C: Various HTT oligonucleotides, including various pan-specific mouse-targeting HTT oligonucleotides, were tested for knockdown of HTT in human iCell neurons in vitro.
Numbers represent % of HTT mRNA left after treatment with oligonucleotides. 100.0 would represent 100% HTT level (0% knockdown) and 0.0 would represent 0% HTT level (100% knock down).

TABLE 54B
Activity of certain oligonucleotides.

| Conc. | WV-21182 | | | | WV-21183 | | | | WV-21184 | | | |
|---|---|---|---|---|---|---|---|---|---|---|---|---|
| 10 | 86.4 | 94.7 | 85.6 | 93.4 | 90.4 | 75.7 | 99.8 | 76.2 | 52.9 | 71.5 | 93.7 | 91.6 |
| 3 | 99.3 | 86.0 | 95.6 | 79.5 | 84.8 | 104.2 | 105.3 | 102.1 | 76.5 | 91.6 | 114.5 | 102.3 |
| 1 | 90.4 | 90.9 | 105.9 | 82.5 | 108.5 | 85.9 | 105.7 | 74.4 | 119.7 | 111.4 | 116.6 | 79.7 |
| Conc. | WV-21185 | | | | WV-21186 | | | | WV-21187 | | | |
| 10 | 55.9 | 79.9 | 84.1 | 79.2 | 76.8 | 107.4 | 97.6 | 112.9 | 80.7 | 87.5 | 95.8 | 80.1 |
| 3 | 90.2 | 85.5 | 81.7 | 72.2 | 89.8 | 84.1 | 104.5 | 71.8 | 77.3 | 88.1 | 85.5 | 81.4 |
| 1 | 81.3 | 80.0 | 109.1 | 66.3 | 94.8 | 69.1 | 84.4 | 68.6 | 79.6 | 87.6 | 113.0 | 79.5 |
| Conc. | WV-21188 | | | | WV-21189 | | | | WV-9679 | | | |
| 10 | 60.3 | 92.1 | 108.7 | 105.4 | 78.4 | 86.1 | 83.7 | 102.2 | 17.3 | 26.2 | 23.3 | 21.3 |
| 3 | 86.9 | 103.0 | 93.2 | 83.6 | 78.6 | 111.7 | 100.1 | 90.7 | 33.0 | 29.9 | 35.5 | 33.6 |
| 1 | 97.0 | 69.8 | 113.8 | 69.2 | 87.1 | 107.8 | 87.5 | 97.9 | 53.2 | 51.3 | 55.6 | 41.3 |

TABLE 54C
Activity of certain oligonucleotides.

| Conc. | WV-21190 | | | | WV-21191 | | | | WV-21192 | | | |
|---|---|---|---|---|---|---|---|---|---|---|---|---|
| 10 | 77.3 | 80.8 | 70.4 | 68.3 | 61.4 | 74.3 | 96.5 | 80.4 | 56.3 | 96.7 | 94.4 | 87.2 |
| 3 | 86.8 | 106.1 | 100.7 | 91.0 | 61.1 | 87.5 | 100.4 | 92.7 | 70.2 | 91.1 | 81.2 | 85.3 |
| 1 | 81.1 | 99.3 | 127.6 | 108.6 | 69.0 | 123.7 | 102.5 | 98.7 | 104.7 | 104.3 | 96.8 | 128.2 |
| Conc. | WV-21193 | | | | WV-21194 | | | | WV-21195 | | | |
| 10 | 50.7 | 96.2 | 83.7 | 75.9 | 74.4 | 94.0 | 102.7 | 95.0 | 45.3 | 88.5 | 83.5 | 107.5 |
| 3 | 92.6 | 75.3 | 106.3 | 86.5 | 95.2 | 111.3 | 109.4 | 93.4 | 71.8 | 128.3 | 108.3 | 98.7 |
| 1 | 97.0 | 105.7 | 91.9 | 80.7 | 115.6 | 98.8 | 106.7 | 114.0 | 74.6 | 150.8 | 106.6 | 137.2 |
| Conc. | WV-21196 | | | | WV-21197 | | | | WV-9679 | | | |
| 10 | 86.2 | 83.1 | 97.1 | 77.3 | 70.6 | 106.2 | 82.0 | 92.0 | 17.3 | 24.1 | 20.9 | 19.5 |
| 3 | 88.0 | 103.7 | 105.4 | 87.0 | 92.3 | 97.2 | 101.6 | 103.5 | 29.1 | 44.2 | 43.0 | 37.1 |
| 1 | 105.7 | 104.3 | 100.8 | 128.2 | 108.1 | 125.3 | 95.5 | 95.9 | 62.3 | 56.4 | 67.6 | 74.7 |

TABLE 56A
Activity of certain oligonucleotides.

| Conc. | WV-21198 | | | | WV-21199 | | | | WV-21200 | | | |
|---|---|---|---|---|---|---|---|---|---|---|---|---|
| 10 | 108.6 | 92.9 | 109.2 | 111.0 | 80.6 | 117.5 | 98.1 | 91.9 | 93.4 | 82.4 | 126.7 | 99.1 |
| 3 | 91.9 | 104.7 | 137.5 | 104.6 | 95.2 | 90.6 | 88.5 | 96.8 | 93.9 | 94.3 | 95.0 | 84.7 |
| 1 | 130.0 | 102.6 | 91.6 | 99.8 | 119.4 | 96.3 | 97.5 | 82.6 | 118.5 | 126.2 | 85.9 | 99.9 |
| Conc. | WV-21201 | | | | WV-21202 | | | | WV-21203 | | | |
| 10 | 77.6 | 87.5 | 101.4 | 99.8 | 87.8 | 101.5 | 103.7 | 110.0 | 82.5 | 96.8 | 118.4 | 111.7 |
| 3 | 82.1 | 104.7 | 101.1 | 109.1 | 105.9 | 103.2 | 105.6 | 101.8 | 111.8 | 111.1 | 97.0 | 115.7 |
| 1 | 94.2 | 98.2 | 91.3 | 87.1 | 101.6 | 100.6 | 82.8 | 91.3 | 98.0 | 146.9 | 91.9 | 85.7 |
| Conc. | WV-21204 | | | | WV-21205 | | | | WV-9679 | | | |
| 10 | 79.8 | 105.9 | 91.9 | 117.1 | 102.9 | 104.9 | 101.7 | 100.9 | 28.7 | 24.3 | 28.4 | 34.7 |
| 3 | 100.6 | 132.6 | 103.4 | 110.9 | 94.0 | 101.9 | 92.2 | 105.8 | 47.6 | 42.0 | 50.8 | 47.7 |
| 1 | 114.8 | 97.3 | 107.2 | 117.7 | 94.7 | 98.6 | 120.5 | 112.8 | 55.2 | 56.6 | 59.7 | 59.5 |

Various HTT oligonucleotides were tested for knockdown of HTT in neurons in vitro.
The concentration of oligonucleotide used is shown as exp 10 in uM. Cells used are homozygous for SNP targeted by oligonucleotides.
Numbers represent % of HTT mRNA left after treatment with oligonucleotides. 100.0 would represent 100% HTT level (0% knockdown) and 0.0 would represent 0% HTT level (100% knock down).

TABLE 56B
Activity of certain oligonucleotides.

| | WV-9666 | | | WV-9693 | | |
|---|---|---|---|---|---|---|
| 0.921 | 75.3 | 73 | 75.7 | 92.4 | 82.4 | 86.6 |
| 0.444 | 94.6 | 87 | 80.7 | 91.9 | 95.2 | 60.1 |
| −0.034 | 85.7 | 74.6 | 77.9 | 39.3 | 87.6 | 94.4 |
| −0.511 | 85.5 | 89.5 | 103.1 | 85.8 | 96.7 | 103 |
| −0.988 | 90.5 | 103.5 | 98.6 | 111.6 | 102.6 | 105.4 |
| −1.465 | 87.4 | 102.5 | 116.2 | 106.3 | 106.9 | 110.1 |
| −1.942 | 108.7 | 107.8 | 115.6 | 111.6 | 110.8 | 116.8 |
| | WV-9679 | | | WV-9491 | | |
| 0.921 | 24.3 | 30.1 | 23.7 | 106.3 | | 81.4 |
| 0.444 | 42.6 | 52.6 | 41.2 | 89.5 | 106.1 | 97.5 |
| −0.034 | 66 | 57.7 | 58.7 | 101.6 | | 94.4 |
| −0.511 | 89.1 | 81.7 | 84.9 | 103.6 | 82.7 | 88.7 |
| −0.988 | 101.4 | 92.7 | 98.3 | 111.8 | 113.4 | 95.4 |
| −1.465 | 94.5 | 104.7 | 110.1 | 112.8 | 102.9 | 110.7 |
| −1.942 | 89.8 | 122.6 | 114.1 | 113.5 | 108.1 | 95 |

Various HTT oligonucleotides were tested for knockdown of HTT in ND0536-1 cells in vitro, with 7 days of treatment, and 7 days of differentiation.
The concentration of oligonucleotide used is shown as exp 10 in M.
Numbers indicate the % of HTT remaining (relative to control) at the indicated oligonucleotide concentrations. 1.00 would represent 100% HTT mRNA remaining (0.0% knockdown); and 0.0 will represent 0.0% HTT mRNA remaining (100.0% knockdown). WV-12890 is a NTC.

TABLE 57
Activity of certain oligonucleotides.

| | | |
|---|---|---|
| WV-9679 | WV-14914 | WV-12890 |

| | | | | | | |
|---|---|---|---|---|---|---|
| −6.505 | 0.786 | 0.949 | 0.837 | 0.787 | 0.684 | 0.740 |
| −6.204 | 0.827 | 0.855 | 0.775 | 0.784 | 0.948 | 1.173 |
| −5.903 | 0.781 | 0.619 | 0.902 | 0.804 | 0.847 | 1.162 |
| −5.602 | 0.767 | 0.665 | 0.371 | 0.690 | 0.933 | 0.953 |
| −5.301 | 0.729 | 0.866 | 0.275 | 0.396 | 1.170 | 0.775 |
| −5.000 | 0.401 | 0.604 | 0.146 | 0.113 | 0.992 | 0.916 |

Various HTT oligonucleotides were tested for knockdown of HTT in iNeurons in vitro.
The concentration of oligonucleotide used is shown as exp 10 in uM (Conc.).
Numbers represent % of HTT mRNA left after treatment with oligonucleotides. 100.0 would represent 100% HTT level (0% knockdown) and 0.0 would represent 0% HTT level (100% knock down).

TABLE 58
Activity of certain oligonucleotides.

| Conc. | WV-21404 | | | | WV-21405 | | | |
|---|---|---|---|---|---|---|---|---|
| 1.301 | 16.8 | 16.5 | 38.9 | 24.5 | 4.6 | 18.8 | 16.9 | 7.6 |
| 0.824 | 45.6 | 11.4 | 22.1 | 26.8 | 23.4 | 34.2 | 13.7 | 29.1 |
| 0.347 | 50.2 | 46.2 | 5.1 | 51.8 | 28.6 | 18.6 | 23.0 | 43.2 |
| −0.130 | 47.0 | 48.9 | 49.6 | 47.1 | 50.6 | 44.0 | 49.3 | 56.0 |
| −0.607 | 107.0 | 81.5 | 125.4 | 57.3 | 94.0 | 109.5 | 80.4 | 76.4 |
| −1.085 | 104.1 | 83.2 | 133.4 | 110.7 | 80.5 | 96.1 | 103.3 | 82.2 |
| −1.562 | 63.8 | 114.5 | 121.4 | 92.6 | | 75.0 | 107.7 | 120.6 |
| Conc. | WV-21406 | | | | WV-21412 | | | |
| 1.301 | 1.3 | 15.8 | 29.8 | 21.1 | 1.2 | 11.4 | 30.9 | 8.8 |
| 0.824 | 24.4 | 18.4 | 15.6 | 26.3 | 17.6 | 0.1 | 18.4 | 9.9 |
| 0.347 | 36.6 | 54.8 | 50.5 | 42.3 | 12.9 | 27.1 | 18.4 | 21.7 |
| −0.130 | 77.2 | 55.5 | 73.7 | 54.5 | 45.4 | 38.3 | 30.0 | 49.7 |
| −0.607 | 99.5 | 71.6 | 87.4 | 119.5 | | 83.7 | 114.5 | 71.5 |
| −1.085 | | 93.5 | 110.4 | 79.2 | 110.1 | 101.9 | 95.3 | 76.3 |
| −1.562 | 96.2 | 119.6 | 101.3 | 111.3 | 108.5 | 110.0 | 108.6 | 91.0 |
| Conc. | WV-23689 | | | | WV-23690 | | | |
| 1.301 | 7.5 | | 3.2 | 13.6 | 6.8 | 9.8 | 8.2 | 6.2 |
| 0.824 | 7.1 | 8.6 | 36.2 | 6.5 | 18.8 | 14.7 | 12.2 | 11.0 |
| 0.347 | 15.1 | 30.7 | 34.5 | 32.6 | 20.9 | 30.3 | 32.0 | 54.8 |
| −0.130 | 38.9 | 51.1 | 55.3 | 52.5 | 53.6 | 51.8 | 38.5 | 79.7 |
| −0.607 | 91.6 | 77.7 | 93.0 | 104.0 | 101.7 | 95.2 | 106.1 | 106.0 |
| −1.085 | 87.7 | 128.1 | 100.2 | 87.7 | 95.8 | 94.8 | 79.1 | 91.9 |
| −1.562 | 66.7 | 114.9 | 116.5 | 106.7 | 116.6 | | 102.7 | 127.2 |
| Conc. | WV-23691 | | | | WV-23692 | | | |
| 1.301 | 14.8 | 23.1 | 26.4 | 36.6 | 5.3 | 12.0 | 24.2 | 12.5 |
| 0.824 | 37.6 | 36.7 | 31.0 | 20.5 | 37.2 | 31.8 | 25.8 | 12.7 |
| 0.347 | 34.1 | 45.7 | | 48.0 | 37.2 | 24.8 | 13.5 | 29.4 |
| −0.130 | 54.6 | 86.9 | 63.7 | 75.2 | 51.7 | 40.0 | 58.5 | 75.8 |
| −0.607 | 109.6 | 91.2 | 88.2 | 91.1 | 96.4 | 86.6 | 87.3 | 84.7 |
| −1.085 | 104.4 | | 90.5 | 79.8 | 104.3 | 84.1 | 79.4 | 84.8 |
| −1.562 | 74.6 | 70.7 | 65.6 | 87.9 | 79.8 | 78.6 | 85.2 | 101.0 |
| Conc. | WV-14914 | | | | WV-9679 | | | |
| 1.301 | 3.7 | 31.9 | 15.3 | 26.9 | 23.1 | 49.0 | 27.9 | 27.8 |
| 0.824 | 40.7 | 26.8 | 15.5 | 23.0 | 35.4 | 52.9 | 39.1 | 64.1 |
| 0.347 | 28.1 | 10.9 | 42.5 | 31.2 | 58.4 | 58.4 | 50.1 | 75.0 |
| −0.130 | 29.9 | 51.5 | 55.9 | 54.3 | 96.6 | | 91.7 | |
| −0.607 | 70.0 | 77.1 | 79.1 | 66.7 | 115.9 | | 93.7 | 86.9 |
| −1.085 | 70.7 | 85.8 | 68.6 | 86.0 | | | 72.4 | 102.2 |
| −1.562 | 78.4 | 77.3 | 101.5 | 72.7 | 98.5 | 78.5 | 120.9 | 102.9 |

Various HTT oligonucleotides were tested for knockdown of HTT in cells in vitro.
Numbers represent % of HTT mRNA left after treatment with oligonucleotides. 100.0 would represent 100% HTT level (0% knockdown) and 0.0 would represent 0% HTT level (100% knock down).

TABLE 59
Activity of certain oligonucleotides.

| | |
|---|---|
| WV-9491 | 100 |
| WV-9679 | 60.6 |
| WV-14914 | 27.4 |
| WV-12282 | 57.4 |
| WV-21260 | 56.5 |
| WV-21261 | 61.2 |
| WV-21262 | 81.6 |
| WV-21264 | 79 |
| WV-21265 | 76.2 |
| WV-21266 | 79 |
| WV-21267 | 29.6 |
| WV-21268 | 39.1 |
| WV-21269 | 60.5 |
| WV-21270 | 54 |
| WV-21271 | 34.6 |
| WV-21272 | 56.5 |
| WV-21273 | 53.5 |
| WV-17782 | 29.3 |
| WV-21274 | 28.4 |
| WV-21275 | 39.9 |
| WV-21276 | 45.6 |
| WV-21277 | 65.1 |
| WV-21278 | 78.2 |
| WV-21279 | 60.2 |
| WV-21280 | 75.9 |
| WV-21281 | 81 |
| WV-21282 | 104.8 |
| WV-21283 | 90.5 |
| WV-21284 | 108.4 |
| WV-21285 | 107.4 |
| WV-21286 | 87.5 |
| WV-21287 | 107.4 |
| WV-21288 | 94.4 |
| WV-21289 | 111.4 |
| WV-21290 | 93.8 |
| WV-21291 | 94.8 |
| WV-21292 | 93.7 |
| WV-21293 | 90.5 |
| WV-21294 | 117.7 |
| WV-21295 | 110.7 |
| WV-21296 | 106.9 |
| WV-21297 | 100.2 |
| WV-21298 | 92 |
| WV-21299 | 96.5 |
| WV-21300 | 108.5 |
| WV-21301 | 111.7 |
| WV-21302 | 53.8 |
| WV-21303 | 97.1 |
| WV-21304 | 66.2 |
| WV-21305 | 103.1 |
| WV-21306 | 71.2 |
| WV-21307 | 112.5 |
| WV-21308 | 63.7 |
| WV-21309 | 102.3 |
| WV-21310 | 69.6 |
| WV-21312 | 80.4 |
| WV-21313 | 123 |
| WV-21314 | 85.3 |
| WV-21315 | 101.2 |
| WV-21316 | 101.9 |
| WV-21317 | 102 |
| WV-21318 | 40 |
| WV-21319 | 100.6 |
| WV-21320 | 44.1 |
| WV-21321 | 100.4 |
| WV-21322 | 57.5 |
| WV-21323 | 109.2 |
| WV-21324 | 52.4 |
| WV-21325 | 113.8 |
| WV-21326 | 88.3 |
| WV-21327 | 110.7 |
| WV-21328 | 92.4 |
| WV-21329 | 98.2 |
| WV-21330 | 66.4 |
| WV-21331 | 103.9 |
| WV-21332 | 103.9 |
| WV-21333 | 109.4 |
| WV-21334 | 104.6 |
| WV-21335 | 117.7 |
| WV-21336 | 86.2 |
| WV-21337 | 88.2 |
| WV-21338 | 70.3 |
| WV-21339 | 112.5 |
| WV-21340 | 110.2 |
| WV-21341 | 113.2 |
| WV-21342 | 95.7 |
| WV-21343 | 99.5 |

Various HTT oligonucleotides were tested for knockdown of HTT in cells in vitro.
Numbers indicate the % of HTT remaining (relative to control) at the indicated oligonucleotide concentrations. 1.00 would represent 100 HTT mRNA remaining (0.06 knockdown); and 0.0 will represent 0.07 HTT mRNA remaining (100.0% knockdown).

TABLE 60
Activity of certain oligonucleotides.

| WV- | WV- | WV- | WV- | WV- | WV- | WV- | WV- | WV- | WV- | WV- |
|---|---|---|---|---|---|---|---|---|---|---|
| 12258 | 12259 | 12260 | 12261 | 12262 | 12263 | 12264 | 12265 | 12266 | 12267 | 12268 |
| 0.845 | 0.627 | 0.702 | 0.573 | 0.855 | 0.906 | 0.681 | 0.507 | 1.015 | 0.902 | 1.146 |
| 0.852 | 0.663 | 0.787 | 0.525 | 0.708 | 0.901 | 0.548 | 0.64 | 0.84 | 1.05 | 0.905 |
| 0.905 | 0.698 | 0.758 | 0.622 | 0.456 | 0.982 | 0.661 | 0.441 | 0.811 | | 1.092 |
| WV- | WV- | WV- | WV- | WV- | WV- | WV- | WV- | WV- | WV- | WV- |
| 12269 | 12270 | 12271 | 12272 | 12273 | 12274 | 12275 | 12276 | 12277 | 12278 | 12279 |
| 0.836 | 1.111 | 0.801 | 0.844 | 1.18 | 0.996 | 1.062 | 0.979 | 0.91 | 0.897 | 0.59 |
| 0.716 | 0.95 | 0.693 | 0.778 | 0.786 | 0.905 | 1.035 | 0.988 | 1.022 | 0.971 | 0.549 |
| 1.032 | 0.871 | 0.804 | 0.78 | 0.915 | 1.07 | 0.968 | 1.011 | 0.953 | 0.822 | 0.659 |

| WV- | WV- | WV- | WV- | WV- | WV- | WV- | WV- | WV- |
|---|---|---|---|---|---|---|---|---|
| 12280 | 12281 | 12282 | 12283 | 12284 | 12285 | 12286 | 12287 | 9679 |
| 0.712 | 0.509 | 0.476 | 1.137 | 0.453 | 0.511 | 0.783 | 0.836 | 0.263 |
| 0.764 | 0.468 | 0.493 | 1.054 | 0.544 | 0.656 | 0.641 | 0.809 | 0.268 |
| 0.75 | 0.382 | | 0.948 | 0.545 | 0.591 | 0.721 | 1.053 | 0.25 |

Various HTT oligonucleotides, including various pan-specific HTT oligonucleotides, were tested for knockdown of HTT in iCell neurons in vitro at 10 uM.
Numbers represent % of HTT mRNA left after treatment with oligonucleotides. 100.0 would represent 100% HTT level (0% knockdown) and 0.0 would represent 000 HTT level (100% knock down).

TABLE 61
Activity of certain oligonucleotides.

| WV- | WV- | WV- | WV- | WV- | WV- | WV- | WV- | WV- | WV- | WV- | WV- |
|---|---|---|---|---|---|---|---|---|---|---|---|
| 10783 | 10784 | 10785 | 10786 | 10787 | 10788 | 10789 | 10790 | 9679 | 9491 | 10791 | 10792 |
| 99.3 | 95.6 | 85.7 | 53.3 | 32.5 | 48.3 | 66.3 | | 17.8 | 104.9 | 41.1 | 77.1 |
| 73.6 | 83.4 | 72.1 | 51.2 | 37.1 | 54.3 | 59.1 | 26.9 | 30.4 | 97.4 | 33.7 | 72.2 |
| 73.4 | 82.5 | 74.8 | 55.6 | 41.1 | 67.2 | 63.8 | 35 | 25.4 | 97.6 | 34.4 | 81.1 |

| WV- | WV- | WV- | WV- | WV- | WV- | WV- | WV- | WV- | WV- | WV- |
|---|---|---|---|---|---|---|---|---|---|---|
| 10793 | 10794 | 10795 | 10796 | 10797 | 10798 | 10799 | 10800 | 10801 | 10802 | 10803 |
| 76 | 70.7 | 62.6 | 79.9 | 92.6 | 100 | 73 | 64.6 | 69.8 | 82.8 | 95.8 |
| 78.7 | 81 | 60.5 | 83.3 | 87.9 | 107.2 | 99.4 | 72.8 | 77.8 | 87 | 86.1 |
| 82.9 | 68.4 | 64.6 | 82.5 | 80.6 | 99.6 | 93.7 | 81.7 | 85.4 | 88.9 | 88.7 |

| WV-10804 | WV-10805 | WV-10806 | WV-10807 | WV-10808 | WV-10809 |
|---|---|---|---|---|---|
| 89.3 | 91 | 46.5 | 87.7 | 69.3 | 81 |
| 78.2 | 78.8 | 43 | 89.8 | 74.7 | 86.8 |
| 77.6 | 78.4 | 51.6 | 105.6 | 73.7 | 98.6 |

| WV-10810 | WV-10811 | WV-10812 | WV-10813 | WV-10814 | WV-10815 | WV-10816 | WV-10817 |
|---|---|---|---|---|---|---|---|
| 47.4 | 44.3 | 72.9 | 79.6 | 107.2 | 58.7 | 73 | 110.5 |
| 51.5 | 47.9 | 78.2 | 71.7 | 108.1 | 57.2 | 77.6 | 87.3 |
| 59.4 | 44.7 | 85.3 | 90.2 | | 58.6 | 70.6 | 93.8 |

### Table 61A and 611B:

Various HTT oligonucleotides were tested for knockdown of HTT in iNeurons cells in vitro.
The concentration of oligonucleotide used is shown as exp 10 in uM.
Numbers indicate the % of HTT remaining (relative to control) at the indicated oligonucleotide concentrations. 1.00 would represent 100% HTT mRNA remaining (0.0% knockdown); and 0.0 will represent 0.00% HTT mRNA remaining (100.0% knockdown).

TABLE 61B
Activity of certain oligonucleotides.

| Conc. | WV-14915 | | | WV-15079 | | | WV-14914 | | |
|---|---|---|---|---|---|---|---|---|---|
| 1.3 | 0.347 | 0.154 | 0.178 | 0.544 | 0.167 | 0.171 | 0.649 | 0.111 | 0.13 |
| 0.82 | 0.705 | 0.233 | 0.207 | 0.891 | 0.285 | 0.119 | 1.106 | 0.181 | 0.106 |
| 0.35 | 1.058 | 0.549 | 0.469 | 1.558 | 0.599 | 0.271 | 0.865 | 0.359 | 0.268 |
| −0.13 | 0.795 | 0.641 | 0.71 | 0.838 | 0.927 | 0.594 | 0.689 | 0.781 | 0.714 |
| −0.61 | 0.861 | 1.042 | 0.732 | 1.002 | 0.879 | 0.769 | 1.032 | 1.161 | 0.729 |
| −1.08 | 0.95 | 1.252 | 0.77 | 1.297 | 1.275 | 0.818 | 1.273 | 1.262 | 0.702 |
| −1.56 | 0.998 | 1.044 | 1.119 | 1.104 | 1.154 | 0.976 | 1.173 | 1.15 | 0.9 |
| −2.04 | 0.872 | 0.965 | 0.961 | 1.015 | 0.958 | 0.88 | 0.947 | 0.954 | 1.046 |

| Conc. | WV-15080 | | | WV-15077 | | |
|---|---|---|---|---|---|---|
| 1.3 | 0.381 | 0.161 | 0.106 | 0.373 | 0.321 | 0.293 |
| 0.82 | 0.517 | 0.259 | −0.031 | 0.83 | 0.586 | 0.456 |
| 0.35 | 0.443 | 0.4 | 0.173 | 0.93 | 0.926 | 0.702 |
| −0.13 | 0.641 | 0.713 | 0.46 | 1.089 | 1.115 | 0.581 |
| −0.61 | 1.083 | 1.046 | 0.617 | 1.39 | 1.586 | 1.078 |
| −1.08 | 1.205 | 1.164 | 0.699 | 1.445 | 1.547 | 1.026 |
| −1.56 | 1.305 | 1.035 | 1.024 | 1.277 | 1.304 | 0.997 |
| −2.04 | 0.994 | 0.887 | 0.915 | 0.967 | 1.175 | 0.877 |

| Conc. | WV-12282 | | | WV-12283 | | |
|---|---|---|---|---|---|---|
| 1.3 | 0.369 | 0.301 | 0.307 | 0.751 | 0.682 | 0.662 |
| 0.82 | 0.628 | 0.641 | 0.403 | 0.992 | 0.972 | 0.867 |
| 0.35 | 0.878 | 0.876 | 0.731 | 1.538 | 1.357 | 1.022 |
| −0.13 | 1.044 | 1.045 | 1.067 | 1.398 | 1.629 | 1.037 |
| −0.61 | 1.257 | 1.281 | 0.982 | 1.478 | | 1.162 |
| −1.08 | 1.103 | 1.299 | 0.918 | 1.268 | 1.637 | 1.063 |
| −1.56 | 1.486 | 1.412 | 1.192 | | 1.482 | 1.095 |
| −2.04 | 0.962 | 1.059 | 1.036 | 0.903 | 1.108 | 1.265 |

| Conc. | WV-12284 | | | WV-15078 | | | WV-9679 | | |
|---|---|---|---|---|---|---|---|---|---|
| 1.3 | 0.438 | 0.413 | 0.321 | 0.439 | 0.366 | 0.334 | 0.279 | 0.295 | 0.172 |
| 0.82 | 0.517 | 0.664 | 0.625 | 0.747 | 0.575 | 0.529 | 0.522 | 0.556 | 0.444 |
| 0.35 | 1.105 | 0.908 | 0.788 | 1.013 | 0.935 | 0.895 | 0.893 | 0.924 | 0.581 |
| −0.13 | 1.307 | 0.796 | 0.932 | 1.406 | 1.08 | 1.031 | 1.268 | 1.209 | 0.821 |
| −0.61 | 1.331 | 1.475 | 0.914 | 1.331 | 1.413 | 1.081 | 1.292 | 1.124 | 0.983 |
| −1.08 | 1.367 | 1.549 | 1.141 | 1.316 | 1.464 | 1.127 | 1.343 | 1.241 | 1.119 |
| −1.56 | 1.403 | 1.339 | 1.107 | | 1.474 | 1.12 | 1.49 | 0.685 | 1.131 |
| −2.04 | 1.089 | 1.004 | | 1.052 | 0.997 | | 1.161 | 1.079 | |

| Conc. | WV-12892 NTC | | |
|---|---|---|---|
| 1 | 1.063 | 0.961 | 0.939 |
| 0.523 | 0.976 | 1.09 | 1.025 |
| 0.046 | 1.021 | 0.87 | 1.016 |
| −0.431 | 0.871 | 1.079 | 1.07 |
| −0.908 | 1.042 | 1.005 | 1.005 |
| −1.386 | 0.965 | | 0.954 |
| −1.863 | 1.06 | | 0.992 |

TABLE 62A
Activity of certain oligonucleotides.

| | WV-14915 | | | WV-15079 | | | WV-14914 | | |
|---|---|---|---|---|---|---|---|---|---|
| 1.3 | 0.221 | 0.186 | 0.145 | 0.183 | 0.183 | 0.172 | 0.107 | 0.112 | 0.087 |
| 0.82 | 0.279 | 0.301 | 0.349 | 0.236 | 0.292 | 0.299 | 0.137 | 0.141 | 0.122 |
| 0.35 | 0.431 | 0.503 | 0.59 | 0.436 | 0.479 | 0.554 | 0.263 | 0.23 | 0.288 |
| −0.13 | 0.642 | 0.678 | 0.694 | 0.586 | 0.625 | 0.656 | 0.495 | 0.441 | 0.568 |
| −0.61 | 0.811 | 0.892 | 0.828 | 0.807 | 0.801 | 0.799 | 0.721 | 0.622 | 0.769 |
| −1.08 | 0.869 | 0.805 | 0.732 | 0.833 | 0.811 | 0.758 | 0.886 | 0.76 | 0.784 |
| −1.56 | 0.918 | 0.87 | 0.811 | 1.036 | 0.915 | 0.849 | 0.943 | 0.787 | 0.858 |
| −2.04 | 1.026 | 0.802 | | 0.985 | 0.835 | | 0.929 | 0.772 | |
| | WV-15080 | | | WV-15077 | | | WV-12282 | | |
| 1.3 | 0.108 | 0.109 | 0.09 | 0.348 | 0.299 | 0.301 | 0.37 | 0.338 | 0.348 |
| 0.82 | 0.128 | 0.13 | 0.129 | 0.529 | 0.41 | 0.476 | 0.547 | 0.496 | 0.552 |
| 0.35 | 0.204 | 0.229 | 0.242 | 0.647 | 0.6 | 0.79 | 0.731 | 0.692 | 0.865 |
| −0.13 | 0.521 | 0.413 | 0.478 | 0.841 | 0.783 | 0.868 | 0.878 | 0.789 | 1.017 |
| −0.61 | 0.696 | 0.605 | 0.723 | 1.01 | 0.82 | 0.964 | 0.994 | 0.804 | 0.936 |
| −1.08 | 0.84 | 0.702 | 0.742 | 0.931 | 0.965 | 0.984 | 0.943 | 0.742 | 1.001 |
| −1.56 | 0.999 | 0.742 | 0.838 | 0.97 | 0.852 | 0.883 | 1.094 | 0.852 | 0.974 |
| −2.04 | 0.988 | 0.747 | | 0.924 | 0.775 | | 0.924 | 0.703 | |
| | WV-12283 | | | WV-12284 | | | WV-15078 SR | | |
| 1.3 | 0.727 | 0.8 | 0.718 | 0.417 | 0.266 | 0.41 | 0.34 | 0.332 | 0.366 |
| 0.82 | 0.883 | 0.773 | 0.829 | 0.607 | 0.521 | 0.592 | 0.55 | 0.49 | 0.573 |
| 0.35 | 0.904 | 0.754 | 1.043 | 0.719 | 0.73 | 0.83 | 0.695 | 0.601 | 0.8 |
| −0.13 | 0.959 | 0.883 | 1.092 | 0.873 | 0.802 | 0.774 | 0.931 | 0.764 | 0.894 |
| −0.61 | 1.027 | 0.79 | 1.098 | 0.964 | 0.78 | 0.389 | 0.964 | 0.741 | 1.003 |
| −1.08 | 0.916 | 0.904 | 1.197 | 0.999 | 0.82 | 1.061 | 0.992 | 0.835 | 1.069 |
| −1.56 | 1.003 | 0.837 | 1.017 | 1.076 | 0.818 | 1.036 | 1.083 | 0.852 | 0.958 |
| −2.04 | 0.772 | 0.759 | | 0.848 | 0.738 | | 1.013 | 0.763 | |
| | WV-9679 | | | WV-12892 NC | | | No Rx | | |
| 1.3 | 0.214 | 0.18 | 0.234 | 0.96 | 0.711 | 0.952 | 0.989 | 0.89 | 1.025 |
| 0.82 | 0.493 | 0.501 | 0.504 | 1.006 | 0.829 | 1.024 | 1.104 | 0.781 | 1.013 |
| 0.35 | 0.665 | 0.583 | 0.659 | 0.965 | 0.813 | 1.005 | 0.992 | 0.74 | 1.006 |
| −0.13 | 0.84 | 0.65 | 0.871 | 1.109 | 0.744 | 1.066 | 1.016 | 0.614 | 1.013 |
| −0.61 | 1.004 | 0.717 | 0.953 | 1.102 | 0.872 | 1.086 | 1.09 | 0.714 | 0.963 |
| −1.08 | 0.956 | 0.918 | 0.983 | 1.044 | 0.871 | 1.047 | 1.031 | 0.813 | 0.956 |
| −1.56 | 1.077 | 0.744 | 1.037 | 1.147 | 1.148 | 1.09 | 1.169 | 0.772 | 1.068 |
| −2.04 | 0.986 | 0.847 | | 1.075 | 0.809 | | | | |

### Table 62A, 62B3, 62C, 62D, and 62E:

Various HTT oligonucleotides were tested for knockdown of HTT in neurons in vitro. Cells used are heterozygous for the SNPs targeted by the oligonucleotides.
The concentration of oligonucleotide used is shown as exp 10 in uM.
Numbers indicate the % of HTT remaining (relative to control) at the indicated oligonucleotide concentrations. 1.00 would represent 100% HTT mRNA remaining (0.0% knockdown); and 0.0 will represent 0.00% HTT mRNA remaining (100.0% knockdown); knockdown of both wt and mt HTT are shown.

TABLE 62B
Activity of certain oligonucleotides.

| | WV-12282 wt | | | | WV-12282 mt | | | |
|---|---|---|---|---|---|---|---|---|
| −7.699 | 0.674 | 0.725 | 0.713 | 0.814 | 0.083 | 0.093 | 0.093 | 0.080 |
| −8.000 | 0.803 | 0.852 | 0.902 | 0.983 | 0.234 | 0.189 | 0.262 | 0.205 |
| −8.301 | 0.800 | 0.889 | 0.905 | 0.966 | 0.418 | 0.357 | 0.496 | 0.353 |
| −8.602 | 0.950 | 0.959 | 0.981 | 0.851 | 0.647 | 0.501 | 0.675 | 0.521 |
| −8.903 | 0.923 | 0.979 | 1.045 | 0.967 | 0.744 | 0.671 | 0.811 | 0.654 |
| −9.204 | 0.998 | 1.001 | 0.998 | 0.973 | 0.839 | 0.801 | 0.955 | 0.722 |
| −9.505 | 1.030 | 0.985 | 1.031 | 0.999 | 0.902 | 0.839 | 0.918 | 0.861 |
| −9.806 | 0.868 | 1.008 | 0.864 | 1.178 | 0.879 | 0.953 | 1.005 | 0.912 |
| −10.107 | 0.835 | 1.036 | 0.898 | 1.069 | 0.940 | 1.188 | 0.981 | 0.998 |

TABLE 62C
Activity of certain oligonucleotides.

| | WV-14914 wt | | | | WV-14914 mt | | | |
|---|---|---|---|---|---|---|---|---|
| −7.69897 | 0.707 | 0.787 | 0.611 | 0.693 | 0.098 | 0.107 | 0.096 | 0.101 |
| −8.000 | 0.936 | 0.872 | 0.879 | 0.874 | 0.204 | 0.219 | 0.233 | 0.187 |
| −8.301 | 0.968 | 1.033 | 0.959 | 0.634 | 0.348 | 0.386 | 0.382 | 0.413 |
| −8.602 | 1.057 | 0.962 | 1.033 | 0.772 | 0.637 | 0.504 | 0.613 | 0.551 |
| −8.903 | 0.907 | 0.946 | 1.105 | 0.886 | 0.657 | 0.675 | 0.774 | 0.716 |
| −9.204 | 0.966 | 0.897 | 1.085 | 0.854 | 0.874 | 0.812 | 0.878 | 0.755 |
| −9.505 | 0.916 | 0.839 | 1.103 | 1.056 | 0.815 | 0.805 | 0.970 | 0.875 |
| −9.806 | 0.900 | 0.954 | 1.040 | 0.916 | 0.852 | 0.889 | 0.922 | 1.066 |
| −10.107 | 0.941 | 1.047 | 0.998 | 1.058 | 0.898 | 0.997 | 0.993 | 1.034 |

TABLE 62D
Activity of certain oligonucleotides.

| | WV-15080 wt | | | | WV-15080 mt | | | |
|---|---|---|---|---|---|---|---|---|
| −7.699 | 0.387 | 0.434 | 0.390 | 0.343 | 0.091 | 0.109 | 0.085 | 0.098 |
| −8.000 | 0.687 | 0.703 | 0.646 | 0.576 | 0.181 | 0.200 | 0.172 | 0.179 |
| −8.301 | 1.125 | 0.970 | 0.913 | 0.770 | 0.445 | 0.443 | 0.345 | 0.452 |
| −8.602 | 1.124 | 0.889 | 1.133 | 0.822 | 0.603 | 0.682 | 0.537 | 0.633 |
| −8.903 | 1.021 | 1.046 | 1.106 | 0.769 | 0.761 | 0.751 | 0.571 | 0.779 |
| −9.204 | 0.952 | 1.020 | 1.034 | 0.891 | 0.929 | 0.792 | 0.869 | 0.800 |
| −9.505 | 0.909 | 1.260 | 0.926 | 0.974 | 0.910 | 0.880 | 0.858 | 0.853 |
| −9.806 | 0.949 | 1.107 | 0.988 | 0.976 | 0.918 | 0.961 | 0.997 | 0.934 |
| −10.107 | 0.927 | 1.054 | 0.978 | 0.979 | 1.199 | 1.111 | 0.934 | 1.028 |

TABLE 63
Activity of certain oligonucleotides.

| | WV-15078 wt | | | | WV-15078 mt | | | |
|---|---|---|---|---|---|---|---|---|
| −7.699 | 0.090 | 0.098 | 0.096 | 0.084 | 0.050 | 0.056 | 0.049 | 0.058 |
| −8.000 | 0.190 | 0.195 | 0.200 | 0.192 | 0.095 | 0.098 | 0.104 | 0.112 |
| −8.301 | 0.531 | 0.425 | 0.395 | 0.418 | 0.278 | 0.263 | 0.274 | 0.257 |
| −8.602 | 0.609 | 0.590 | 0.640 | 0.563 | 0.434 | 0.446 | 0.475 | 0.455 |
| −8.903 | 0.693 | 0.789 | 0.816 | 0.679 | 0.648 | 0.598 | 0.635 | 0.647 |
| −9.204 | 0.736 | 0.835 | 0.856 | 0.748 | 0.828 | 0.708 | 0.770 | 0.726 |
| −9.505 | 0.910 | 1.092 | 0.978 | 0.815 | 0.891 | 0.793 | 0.899 | 0.822 |
| −9.806 | 0.882 | 1.135 | 0.982 | 0.934 | 1.016 | 0.866 | 0.951 | 0.960 |
| −10.107 | 1.015 | 1.065 | 0.984 | 0.928 | 1.253 | 1.079 | 1.118 | 1.026 |

Various HTT oligonucleotides were tested for knockdown of HTT in iCell neurons in vitro, with 7 day treatment.
Numbers indicate the % of HTT remaining (relative to control) at the indicated oligonucleotide concentrations. 1.00 would represent 100% HTT mRNA remaining (0.0% knockdown); and 0.0 will represent 0.0% HTT mRNA remaining (100.0% knockdown); knockdown of wild type HTT and mutant HTT are shown.

| Oligonucleotide | Conc. | | wild type (G) | | | mutant (A) | | |
|---|---|---|---|---|---|---|---|---|
| NTC | 10 | uM | 0.877 | 1.216 | 0.908 | 0.980 | 1.208 | 0.812 |
| WV-12282 | 0.1 | uM | 1.097 | 0.995 | 0.804 | 0.747 | 0.747 | 0.703 |
| | 1 | uM | 1.127 | 1.124 | 1.065 | 0.692 | 0.645 | 0.664 |
| | 10 | uM | 2.038 | 0.554 | 1.273 | 0.486 | 0.351 | 0.379 |
| WV-12283 | 0.1 | uM | 0.524 | 0.998 | 0.599 | 0.496 | 0.844 | 0.685 |
| | 1 | uM | 1.211 | 0.975 | 0.987 | 0.926 | 0.754 | 0.693 |
| | 10 | uM | 0.736 | 0.503 | 0.907 | 0.513 | 0.605 | 0.432 |
| WV-14914 | 0.1 | uM | 0.743 | 0.638 | 1.313 | 0.565 | 0.537 | 0.879 |
| | 1 | uM | 1.213 | 0.961 | 0.607 | 0.457 | 0.453 | 0.468 |
| | 10 | uM | 0.823 | 0.565 | 1.062 | 0.220 | 0.225 | 0.243 |
| WV-15078 | 0.1 | uM | 0.864 | 1.154 | 1.086 | 0.686 | 0.844 | 0.836 |
| | 1 | uM | 1.718 | 0.886 | 1.378 | 0.545 | 0.391 | 0.528 |
| | 10 | uM | 0.674 | 0.474 | 0.635 | 0.229 | 0.217 | 0.229 |
| WV-15080 | 0.1 | uM | 1.000 | 1.157 | 1.165 | 0.803 | 0.761 | 0.809 |
| | 1 | uM | 0.926 | 0.939 | 1.639 | 0.448 | 0.453 | 0.625 |
| | 10 | uM | 0.413 | 0.418 | 0.191 | 0.319 | 0.322 | 0.205 |

In addition to these experiments, WV-10787, WV-10790, WV-21178, WV-21179, WV-21180, and WV-21181 were all confirmed to decrease the amount of expression of muHTT, with no, little, or significantly less effect on expression of wt HTT (data not shown); thus, they were all shown to mediate allele-specific knockdown.

### Table 64. Activity of Certain Oligonucleotides.

This table presents a compilation of data from several experiments wherein the efficacy of various HTT oligonucleotides was tested in neurons in vitro.
Various HTT oligonucleotides were tested for knockdown of HTT in neurons in vitro.
Oligonucleotides were delivered at the indicated concentrations. Numbers (% HTT) represent % of HTT remaining, wherein 100.0 would represent 100.0% HTT remaining (0.0% knockdown) and 0.0% would represent 0.0% HTT remaining (100.0% knockdown).
Replicates of various experiments are shown. Not all controls are necessarily shown.

| Oligonucleotide | Conc. (uM) | % HTT |
|---|---|---|
| WV-22946 | 20 | 30.1 |
| WV-12890 | 20 | 128.8 |
| WV-12891 | 20 | 126.4 |
| WV-12892 | 20 | 125.7 |
| WV-14914 | 20 | 14.4 |
| WV-22920 | 20 | 118.8 |
| WV-22921 | 20 | 108.0 |
| WV-22922 | 20 | 97.3 |
| WV-22923 | 20 | 79.2 |
| WV-22924 | 20 | 91.3 |
| WV-22925 | 20 | 99.3 |
| WV-22926 | 20 | 75.1 |
| WV-22927 | 20 | 87.9 |
| WV-22928 | 20 | 78.8 |
| WV-22929 | 20 | 70.1 |
| WV-22930 | 20 | 75.0 |
| WV-22931 | 20 | 60.3 |
| WV-22932 | 20 | 100.8 |
| WV-22933 | 20 | 110.7 |
| WV-22934 | 20 | 84.4 |
| WV-22935 | 20 | 125.9 |
| WV-22936 | 20 | 116.1 |
| WV-22937 | 20 | 97.6 |
| WV-22938 | 20 | 110.5 |
| WV-22939 | 20 | 113.5 |
| WV-22940 | 20 | 106.3 |
| WV-22941 | 20 | 72.3 |
| WV-22942 | 20 | 89.1 |
| WV-22943 | 20 | 104.9 |
| WV-22944 | 20 | 60.0 |
| WV-22945 | 20 | 80.7 |
| WV-22946 | 20 | 90.7 |
| WV-22947 | 20 | 89.8 |
| WV-22948 | 20 | 96.1 |
| WV-22949 | 20 | 88.9 |
| WV-22950 | 20 | 123.1 |
| WV-22951 | 20 | 115.1 |
| WV-22952 | 20 | 79.5 |
| WV-22953 | 20 | 124.5 |
| WV-22954 | 20 | 103.4 |
| WV-22955 | 20 | 86.0 |
| WV-22956 | 20 | 102.6 |
| WV-22957 | 20 | 102.0 |
| WV-22958 | 20 | 101.7 |
| WV-22959 | 20 | 85.5 |
| WV-22960 | 20 | 95.3 |
| WV-22961 | 20 | 95.0 |
| WV-22962 | 20 | 58.8 |
| WV-22963 | 20 | 79.1 |
| WV-22964 | 20 | 72.5 |
| WV-22965 | 20 | 65.3 |
| WV-22966 | 20 | 76.2 |
| WV-22967 | 20 | 60.8 |
| WV-22968 | 20 | 101.1 |
| WV-22969 | 20 | 116.2 |
| WV-22970 | 20 | 81.8 |
| WV-22971 | 20 | 105.2 |
| WV-22972 | 20 | 81.4 |
| WV-22973 | 20 | 101.8 |
| WV-9679 | 20 | 23.6 |
| mock | 0 | 100.0 |
| WV-12890 | 20 | 102.3 |
| WV-12891 | 20 | 92.2 |
| WV-12892 | 20 | 98.7 |
| WV-14914 | 20 | 10.9 |
| WV-22974 | 20 | 105.1 |
| WV-22975 | 20 | 82.9 |
| WV-22976 | 20 | 91.1 |
| WV-22977 | 20 | 72.2 |
| WV-22978 | 20 | 71.5 |
| WV-22979 | 20 | 72.4 |
| WV-22980 | 20 | 59.2 |
| WV-22981 | 0 | 75.6 |
| WV-22982 | 20 | 70.5 |
| WV-22983 | 20 | 57.5 |
| WV-22984 | 20 | 64.9 |
| WV-22985 | 20 | 64.0 |
| WV-22986 | 20 | 66.2 |
| WV-22987 | 20 | 77.0 |
| WV-22988 | 20 | 65.3 |
| WV-22989 | 20 | 83.6 |
| WV-22990 | 20 | 97.0 |
| WV-22991 | 20 | 75.2 |
| WV-22992 | 20 | 88.1 |
| WV-22993 | 20 | 83.7 |
| WV-22994 | 20 | 68.8 |
| WV-22995 | 20 | 52.1 |
| WV-22996 | 20 | 78.9 |
| WV-22997 | 20 | 77.6 |
| WV-22998 | 20 | 56.5 |
| WV-22999 | 20 | 67.0 |
| WV-23000 | 20 | 66.2 |
| WV-23001 | 20 | 56.2 |
| WV-23002 | 20 | 65.5 |
| WV-23003 | 20 | 41.3 |
| WV-23004 | 20 | 57.3 |
| WV-23005 | 20 | 57.0 |
| WV-23006 | 20 | 57.4 |
| WV-23007 | 20 | 79.1 |
| WV-23008 | 20 | 64.2 |
| WV-23009 | 20 | 65.6 |
| WV-23010 | 20 | 74.1 |
| WV-23011 | 20 | 65.0 |
| WV-23012 | 20 | 61.3 |
| WV-23013 | 20 | 50.7 |
| WV-23014 | 20 | 59.1 |
| WV-23015 | 20 | 69.3 |
| WV-23016 | 20 | 40.0 |
| WV-23017 | 20 | 58.1 |
| WV-23018 | 20 | 53.9 |
| WV-23019 | 20 | 72.9 |
| WV-23020 | 20 | 79.4 |
| WV-23021 | 20 | 69.8 |
| WV-23022 | 20 | 80.2 |
| WV-23023 | 20 | 84.9 |
| WV-23024 | 20 | 69.8 |
| WV-23025 | 20 | 70.3 |
| WV-23026 | 20 | 80.0 |
| WV-23027 | 20 | 55.3 |
| WV-23028 | 20 | 86.2 |
| WV-23029 | 20 | 106.9 |
| WV-23030 | 20 | 111.0 |
| WV-23031 | 20 | 108.4 |
| WV-23032 | 20 | 111.7 |
| WV-23033 | 20 | 92.7 |
| WV-23034 | 20 | 91.9 |
| WV-23035 | 20 | 75.7 |
| WV-23036 | 20 | 82.3 |
| WV-23037 | 20 | 87.9 |
| WV-23038 | 20 | 104.2 |
| WV-23039 | 20 | 88.1 |
| WV-23040 | 20 | 73.8 |
| WV-23041 | 20 | 105.4 |
| WV-23042 | 20 | 92.4 |
| WV-23043 | 20 | 104.5 |
| WV-23044 | 20 | 93.6 |
| WV-23045 | 20 | 85.1 |
| WV-9679 | 20 | 17.0 |
| mock | 0 | 100.0 |
| WV-12890 | 20 | 93.8 |
| WV-12891 | 20 | 96.9 |
| WV-12892 | 20 | 88.8 |
| WV-14914 | 20 | 11.4 |
| WV-22920 | 20 | 95.9 |
| WV-22921 | 20 | 68.7 |
| WV-22922 | 20 | 82.2 |
| WV-22923 | 20 | 79.4 |
| WV-22924 | 20 | 81.8 |
| WV-22925 | 20 | 86.8 |
| WV-22926 | 20 | 33.9 |
| WV-22927 | 20 | 49.5 |
| WV-22928 | 20 | 54.9 |
| WV-22929 | 20 | 51.4 |
| WV-22930 | 20 | 53.6 |
| WV-22931 | 20 | 53.6 |
| WV-22932 | 20 | 45.6 |
| WV-22933 | 20 | 36.6 |
| WV-22934 | 20 | 29.8 |
| WV-22935 | 20 | 45.5 |
| WV-22936 | 20 | 46.5 |
| WV-22937 | 20 | 27.5 |
| WV-22941 | 20 | 46.9 |
| WV-22942 | 20 | 65.4 |
| WV-22943 | 20 | 76.9 |
| WV-22944 | 20 | 28.3 |
| WV-22945 | 20 | 25.2 |
| WV-30115 | 20 | 62.8 |
| WV-22948 | 20 | 34.4 |
| WV-22949 | 20 | 24.1 |
| WV-22950 | 20 | 56.9 |
| WV-22951 | 20 | 43.5 |
| WV-22952 | 20 | 29.4 |
| WV-22953 | 20 | 44.0 |
| WV-22954 | 20 | 71.4 |
| WV-22955 | 20 | 44.4 |
| WV-22956 | 20 | 60.6 |
| WV-22958 | 20 | 56.1 |
| WV-22959 | 20 | 38.1 |
| WV-22960 | 20 | 52.7 |
| WV-22961 | 20 | 51.6 |
| WV-22962 | 20 | 36.0 |
| WV-22963 | 20 | 35.8 |
| WV-22964 | 20 | 37.6 |
| WV-22965 | 20 | 52.3 |
| WV-22966 | 20 | 50.6 |
| WV-22967 | 20 | 51.8 |
| WV-22968 | 20 | 69.1 |
| WV-22969 | 20 | 63.0 |
| WV-22970 | 20 | 35.9 |
| WV-22971 | 20 | 71.6 |
| WV-22972 | 20 | 58.1 |
| WV-22973 | 20 | 52.9 |
| WV-9679 | 20 | 12.1 |
| mock | 0 | 100.0 |
| WV-12890 | 20 | 84.4 |
| WV-12891 | 20 | 109.2 |
| WV-12892 | 20 | 42.2 |
| WV-14914 | 20 | 10.6 |
| WV-22974 | 20 | 102.5 |
| WV-22975 | 20 | 87.8 |
| WV-22976 | 20 | 86.9 |
| WV-22977 | 20 | 64.2 |
| WV-22978 | 20 | 83.1 |
| WV-22979 | 20 | 80.6 |
| WV-22980 | 20 | 74.8 |
| WV-22981 | 20 | 97.1 |
| WV-22982 | 20 | 59.7 |
| WV-22983 | 20 | 62.9 |
| WV-22984 | 20 | 71.3 |
| WV-22985 | 20 | 59.3 |
| WV-22986 | 20 | 70.4 |
| WV-22987 | 20 | 89.2 |
| WV-22988 | 20 | 55.3 |
| WV-22989 | 20 | 122.8 |
| WV-22990 | 20 | 112.7 |
| WV-22991 | 20 | 94.7 |
| WV-22992 | 20 | 88.2 |
| WV-22993 | 20 | 89.1 |
| WV-22994 | 20 | 78.5 |
| WV-22995 | 20 | 54.2 |
| WV-22996 | 20 | 74.9 |
| WV-22997 | 20 | 77.4 |
| WV-22998 | 20 | 52.7 |
| WV-22999 | 20 | 56.4 |
| WV-23000 | 20 | 57.3 |
| WV-23001 | 20 | 75.6 |
| WV-23002 | 20 | 78.7 |
| WV-23003 | 20 | 88.8 |
| WV-23004 | 20 | 74.5 |
| WV-23005 | 20 | 97.0 |
| WV-23006 | 20 | 49.1 |
| WV-23007 | 20 | 76.0 |
| WV-23008 | 20 | 81.4 |
| WV-23009 | 20 | 71.5 |
| WV-23010 | 20 | 94.9 |
| WV-23011 | 20 | 75.3 |
| WV-23012 | 20 | 87.6 |
| WV-23013 | 20 | 77.7 |
| WV-23014 | 20 | 74.5 |
| WV-23015 | 20 | 73.3 |
| WV-23016 | 20 | 34.8 |
| WV-23017 | 20 | 59.6 |
| WV-23018 | 20 | 39.7 |
| WV-23019 | 20 | 46.7 |
| WV-23020 | 20 | 71.0 |
| WV-23021 | 20 | 40.1 |
| WV-23022 | 20 | 72.9 |
| WV-23023 | 20 | 68.3 |
| WV-23024 | 20 | 66.7 |
| WV-23025 | 0 | 136.9 |
| WV-23026 | 20 | 73.0 |
| WV-23027 | 20 | 72.0 |
| WV-23028 | 20 | 71.2 |
| WV-23029 | 20 | 69.4 |
| WV-23030 | 20 | 94.5 |
| WV-23031 | 20 | 41.2 |
| WV-23032 | 20 | 55.3 |
| WV-23033 | 20 | 42.9 |
| WV-23034 | 20 | 13.3 |
| WV-23035 | 20 | 33.3 |
| WV-23036 | 20 | 37.2 |
| WV-23037 | 20 | 72.8 |
| WV-23038 | 20 | 89.4 |
| WV-23039 | 20 | 75.4 |
| WV-23040 | 20 | 83.9 |
| WV-23041 | 20 | 76.3 |
| WV-23042 | 20 | 33.5 |
| WV-23043 | 20 | 66.5 |
| WV-23044 | 20 | 60.9 |
| WV-23045 | 20 | 48.5 |
| WV-9679 | 20 | 14.4 |
| mock | 0 | 100.0 |
| WV-12890 | 20 | 111.2 |
| WV-14914 | 20 | 49.8 |
| WV-30057 | 20 | 111.8 |
| WV-30058 | 20 | 67.1 |
| WV-30059 | 20 | 79.6 |
| WV-30060 | 20 | 85.0 |
| WV-30061 | 20 | 91.7 |
| WV-30062 | 20 | 105.3 |
| WV-30063 | 20 | 90.4 |
| WV-30064 | 20 | 92.7 |
| WV-30065 | 20 | 102.0 |
| WV-30066 | 20 | 99.6 |
| WV-30067 | 20 | 83.7 |
| WV-30068 | 20 | 98.4 |
| WV-30069 | 20 | 97.7 |
| WV-30070 | 20 | 92.5 |
| WV-30071 | 20 | 94.1 |
| WV-30072 | 20 | 83.0 |
| WV-30073 | 20 | 99.0 |
| WV-30074 | 20 | 105.6 |
| WV-30075 | 20 | 86.3 |
| WV-30076 | 20 | 86.0 |
| WV-30077 | 20 | 90.0 |
| WV-30078 | 20 | 73.8 |
| WV-30079 | 20 | 52.3 |
| WV-30080 | 20 | 81.0 |
| WV-30081 | 20 | 71.0 |
| WV-30082 | 20 | 62.2 |
| WV-30083 | 20 | 77.9 |
| WV-30084 | 20 | 59.8 |
| WV-30085 | 20 | 60.0 |
| WV-30086 | 20 | 83.8 |
| WV-30087 | 0 | 60.2 |
| WV-30088 | 20 | 76.4 |
| WV-30089 | 20 | 87.2 |
| WV-30090 | 20 | 73.1 |
| WV-30091 | 20 | 74.5 |
| WV-30092 | 20 | 69.9 |
| WV-30093 | 20 | 78.6 |
| WV-30094 | 20 | 73.5 |
| WV-30095 | 20 | 82.4 |
| WV-30096 | 20 | 87.2 |
| WV-30097 | 20 | 83.3 |
| WV-30098 | 20 | 91.1 |
| WV-30099 | 20 | 61.5 |
| WV-30100 | 20 | 77.8 |
| WV-30101 | 20 | 101.1 |
| WV-30102 | 20 | 76.4 |
| WV-30103 | 20 | 90.7 |
| WV-30104 | 20 | 98.0 |
| WV-30105 | 20 | 86.7 |
| WV-30106 | 20 | 90.2 |
| WV-30107 | 20 | 96.8 |
| WV-30108 | 20 | 77.9 |
| WV-30109 | 20 | 109.6 |
| WV-30110 | 20 | 87.2 |
| WV-30111 | 20 | 75.9 |
| WV-30112 | 20 | 73.2 |
| WV-30113 | 20 | 85.2 |
| WV-30114 | 20 | 71.1 |
| WV-30116 | 20 | 70.0 |
| WV-30117 | 20 | 86.8 |
| WV-30118 | 20 | 84.6 |
| WV-30119 | 20 | 87.0 |
| WV-30120 | 20 | 103.3 |
| WV-30121 | 20 | 91.2 |
| WV-30122 | 20 | 103.4 |
| WV-30123 | 20 | 98.6 |
| WV-30124 | 20 | 91.6 |
| WV-30125 | 20 | 117.4 |
| WV-30126 | 20 | 103.0 |
| WV-30127 | 20 | 105.7 |
| WV-30128 | 20 | 119.4 |
| WV-30129 | 20 | 120.8 |
| WV-30130 | 20 | 112.5 |
| WV-30131 | 20 | 118.2 |
| WV-30132 | 20 | 91.7 |
| WV-9679 | 20 | 70.4 |
| mock | 0 | 100.0 |
| WV-12890 | 20 | 112.3 |
| WV-14914 | 20 | 63.1 |
| WV-30133 | 20 | 104.2 |
| WV-30134 | 20 | 101.0 |
| WV-30135 | 20 | 101.9 |
| WV-30136 | 20 | 100.5 |
| WV-30137 | 20 | 114.1 |
| WV-30138 | 20 | 108.2 |
| WV-30139 | 20 | 97.2 |
| WV-30140 | 20 | 101.1 |
| WV-30141 | 20 | 96.6 |
| WV-30142 | 20 | 75.6 |
| WV-30143 | 20 | 101.6 |
| WV-30144 | 20 | 104.0 |
| WV-30145 | 20 | 110.4 |
| WV-30146 | 20 | 115.6 |
| WV-30147 | 20 | 96.3 |
| WV-30148 | 20 | 88.9 |
| WV-30149 | 20 | 90.9 |
| WV-30150 | 20 | 104.0 |
| WV-30151 | 20 | 100.1 |
| WV-30152 | 20 | 113.2 |
| WV-30153 | 20 | 105.6 |
| WV-30154 | 20 | 103.7 |
| WV-30155 | 20 | 94.9 |
| WV-30156 | 20 | 88.5 |
| WV-30157 | 20 | 89.4 |
| WV-30158 | 20 | 113.1 |
| WV-30159 | 20 | 104.6 |
| WV-30160 | 20 | 99.0 |
| WV-30161 | 20 | 119.5 |
| WV-30162 | 20 | 87.1 |
| WV-30163 | 20 | 66.3 |
| WV-30164 | 20 | 77.4 |
| WV-30165 | 20 | 66.1 |
| WV-30166 | 20 | 74.6 |
| WV-30167 | 20 | 95.8 |
| WV-30168 | 20 | 51.1 |
| WV-30169 | 20 | 61.2 |
| WV-30170 | 20 | 89.6 |
| WV-30171 | 20 | 65.2 |
| WV-30172 | 20 | 76.4 |
| WV-30173 | 20 | 103.5 |
| WV-30174 | 20 | 91.1 |
| WV-30175 | 20 | 78.6 |
| WV-30176 | 20 | 96.6 |
| WV-30177 | 20 | 79.3 |
| WV-30178 | 20 | 86.1 |
| WV-30179 | 20 | 107.3 |
| WV-30180 | 20 | 93.8 |
| WV-30181 | 20 | 87.7 |
| WV-30182 | 20 | 96.9 |
| WV-30183 | 20 | 84.9 |
| WV-30184 | 20 | 77.7 |
| WV-30185 | 20 | 86.5 |
| WV-30186 | 20 | 77.1 |
| WV-30187 | 20 | 86.3 |
| WV-30188 | 20 | 97.2 |
| WV-30189 | 20 | 77.9 |
| WV-30190 | 20 | 90.5 |
| WV-30191 | 20 | 94.2 |
| WV-30192 | 20 | 70.6 |
| WV-30193 | 20 | 77.2 |
| WV-30194 | 20 | 103.5 |
| WV-30195 | 20 | 71.9 |
| WV-30196 | 20 | 82.4 |
| WV-30197 | 20 | 84.9 |
| WV-30198 | 20 | 70.1 |
| WV-30199 | 20 | 55.7 |
| WV-30200 | 20 | 71.2 |
| WV-30201 | 20 | 86.3 |
| WV-30202 | 20 | 92.7 |
| WV-30203 | 20 | 98.4 |
| WV-9679 | 20 | 72.9 |
| mock | 0 | 100.0 |
| WV-12890 | 20 | 111.5 |
| WV-12892 | 20 | 133.4 |
| WV-14914 | 20 | 21.5 |
| WV-22956 | 20 | 100.4 |
| WV-22957 | 20 | 97.2 |
| WV-22958 | 20 | 63.5 |
| WV-22960 | 20 | 64.0 |
| WV-22962 | 20 | 52.7 |
| WV-22963 | 20 | 64.3 |
| WV-22964 | 20 | 52.5 |
| WV-31627 | 20 | 65.9 |
| WV-31628 | 20 | 85.5 |
| WV-31629 | 20 | 80.5 |
| WV-31814 | 20 | 101.7 |
| WV-31815 | 20 | 86.1 |
| WV-31816 | 20 | 101.4 |
| WV-31817 | 20 | 77.2 |
| WV-31818 | 20 | 87.3 |
| WV-31819 | 20 | 98.2 |
| WV-31820 | 20 | 46.7 |
| WV-31821 | 20 | 67.2 |
| WV-31822 | 20 | 68.2 |
| WV-31823 | 20 | 74.7 |
| WV-31824 | 20 | 69.9 |
| WV-31825 | 20 | 71.4 |
| WV-31826 | 20 | 94.8 |
| WV-31827 | 20 | 68.3 |
| WV-31828 | 20 | 40.0 |
| WV-31829 | 20 | 78.1 |
| WV-31830 | 20 | 101.6 |
| WV-31831 | 20 | 110.5 |
| WV-31832 | 20 | 94.3 |
| WV-31833 | 20 | 82.1 |
| WV-31834 | 20 | 93.9 |
| WV-31835 | 20 | 87.6 |
| WV-31836 | 20 | 98.9 |
| WV-31837 | 20 | 85.4 |
| WV-31838 | 20 | 86.8 |
| WV-31839 | 20 | 71.4 |
| WV-31840 | 20 | 57.7 |
| WV-31841 | 20 | 74.1 |
| WV-31842 | 20 | 75.0 |
| WV-31843 | 20 | 70.0 |
| WV-31844 | 20 | 81.9 |
| WV-31845 | 20 | 63.5 |
| WV-31846 | 20 | 74.5 |
| WV-31847 | 20 | 94.9 |
| WV-31848 | 20 | 95.0 |
| WV-31849 | 20 | 95.3 |
| WV-31850 | 20 | 113.5 |
| WV-31851 | 20 | 68.9 |
| WV-31852 | 20 | 85.9 |
| WV-31853 | 20 | 93.0 |
| WV-31854 | 20 | 89.4 |
| WV-31855 | 20 | 112.2 |
| WV-31856 | 20 | 103.4 |
| WV-31857 | 20 | 79.8 |
| WV-31858 | 20 | 99.2 |
| WV-31859 | 20 | 75.2 |
| WV-31860 | 20 | 99.3 |
| WV-31861 | 20 | 97.9 |
| WV-31862 | 20 | 75.3 |
| WV-31863 | 20 | 89.7 |
| WV-31864 | 20 | 112.1 |
| WV-31865 | 20 | 68.3 |
| WV-31866 | 20 | 74.7 |
| WV-31867 | 20 | 31.8 |
| WV-31868 | 20 | 36.0 |
| WV-31869 | 20 | 58.5 |
| WV-9679 | 20 | 52.6 |
| mock | 0 | 100.0 |

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

### Embodiments

1. An oligonucleotide, wherein:

(a) the oligonucleotide targets SNP rs362273, and the base sequence of the oligonucleotide comprises at least 15 contiguous bases, including the SNP position, of the base sequence GTTGATCTGTAGCAGCAGCT, wherein each T can be independently replaced with U;

(b) the oligonucleotide targets SNP rs362272, and the base sequence of the oligonucleotide comprises at least 15 contiguous bases, including the SNP position, of the base sequence ACATAGAGGACGCCGTGCAG, AGAGGACGCCGTGCAGGGCT, ATAGAGGACGCCGTGCAGGG, CACATAGAGGACGCCGTGCA, CATAGAGGACGCCGTGCAGG, GCACATAGAGGACGCCGTGC, or TAGAGGACGCCGTGCAGGGC, wherein each T can be independently replaced with U;

(c) the oligonucleotide targets SNP rs362273, and the base sequence of the oligonucleotide comprises at least 15 contiguous bases, including the SNP position, of the base sequence AGCTGCTGCTACAGATCAAC, AGCTGCTGCTGCAGATCAAC, GGTTGATCTGTAGCAGCAGCT, GTTGATCTGTAGCAGCAGCT, or TTGATCTGTAGCAGCAGCT, wherein each T can be independently replaced with U;

(d) the oligonucleotide targets SNP rs362307, and the base sequence of the oligonucleotide comprises at least 15 contiguous bases, including the SNP position, of the base sequence GGCACAAGGGCACAGAC, GGCACAAGGGCACAGACT, or GGCACAAGGGCACAGACTT, wherein each T can be independently replaced with U;

(e) the oligonucleotide targets SNP rs362331, and the base sequence of the oligonucleotide comprises at least 15 contiguous bases, including the SNP position, of the base sequence GTGCACACAGTAGATGAGGG, wherein each T can be independently replaced with U; or

(f) the oligonucleotide targets SNP rs363099, and the base sequence of the oligonucleotide comprises at least 15 contiguous bases, including the SNP position, of the base sequence AAGGCTGAGCGGAGAAACCC, AGGCTGAGCGGAGAAACCCT, CAAGGCTGAGCGGAGAAACC, CTGAGCGGAGAAACCCTCCA, GCTGAGCGGAGAAACCCTCC, GGCTGAGCGGAGAAACCCTC, or TGAGCGGAGAAACCCTCCAA, wherein each T can be independently replaced with U; and

wherein the oligonucleotide comprises one or more chiral internucleotidic linkages.

2. The oligonucleotide of embodiment 1, wherein the base sequence of the oligonucleotide comprises or is:

(a) GTTGATCTGTAGCAGCAGCT, wherein each T can be independently replaced with U;

(b) ACATAGAGGACGCCGTGCAG, AGAGGACGCCGTGCAGGGCT, ATAGAGGACGCCGTGCAGGG, CACATAGAGGACGCCGTGCA, CATAGAGGACGCCGTGCAGG, GCACATAGAGGACGCCGTGC, or TAGAGGACGCCGTGCAGGGC, wherein each T can be independently replaced with U;

(c) AGCTGCTGCTACAGATCAAC, AGCTGCTGCTGCAGATCAAC, GGTTGATCTGTAGCAGCAGCT, GTTGATCTGTAGCAGCAGCT, or TTGATCTGTAGCAGCAGCT, wherein each T can be independently replaced with U;

(d) GGCACAAGGGCACAGAC, GGCACAAGGGCACAGACT, or GGCACAAGGGCACAGACTT, wherein each T can be independently replaced with U;

(e) GTGCACACAGTAGATGAGGG, wherein each T can be independently replaced with U; or

(f) AAGGCTGAGCGGAGAAACCC, AGGCTGAGCGGAGAAACCCT, CAAGGCTGAGCGGAGAAACC, CTGAGCGGAGAAACCCTCCA, GCTGAGCGGAGAAACCCTCC, GGCTGAGCGGAGAAACCCTC, or TGAGCGGAGAAACCCTCCAA, wherein each T can be independently replaced with U.

3. The oligonucleotide of embodiment 1 or 2, wherein each internucleotidic linkage of the oligonucleotide is independently a natural phosphate linkage, a phosphorothioate linkage, or a

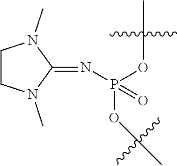 [SMILES: *OP(=O)(N=C1N(C)CCN1C)O*]

(n001) linkage.
4. The oligonucleotide of embodiment 1 or 2, wherein the oligonucleotide comprises one or more natural phosphate linkages, one or more Sp phosphorothioate linkages, and one or more Rp n001 linkages.
5. The oligonucleotide of any one of embodiments 1-4, wherein the oligonucleotide comprises or consists of: a 5′-wing and a 3′-wing, each of which independently comprises one or more modified sugars, and a core between the 5′-wing and the 3′-wing.
6. The oligonucleotide of embodiment 5, wherein the oligonucleotide comprises a 5′-wing comprising 5 consecutive 2′-OMe modified sugars and a 3′-wing comprising 5 consecutive 2′-OMe modified sugars.
7. The oligonucleotide of any one of embodiments 5-6, wherein the core comprises one or more unmodified natural DNA sugars.
8. An oligonucleotide, wherein the oligonucleotide is WV-21404, WV-21405, WV-21406, WV-21412, WV-12282, WV-12283, WV-12284, WV-19840, WV-21178, WV-21179, WV-21180, WV-21181, WV-21403, WV-21409, WV-21410, WV-21447, WV-21448, WV-23689, WV-23690, WV-23691, WV-23692, WV-28152, WV-28153, WV-28154, WV-28155, WV-28157, WV-28158, WV-28159, WV-28160, WV-28161, WV-28162, WV-28163, WV-28164, WV-28165, WV-28166, WV-28167, or WV-28168.
9. The oligonucleotide of any one of the preceding embodiments, wherein the oligonucleotide is in the form of a pharmaceutically acceptable salt.
10. The oligonucleotide of any one of the preceding embodiments, wherein the oligonucleotide is in a sodium salt form.
11. The oligonucleotide of any one of the preceding embodiments, wherein the oligonucleotide is at least about 10%, 20%, 30%, 40%, 50%, 55%, 60%, 65%, 70%, 75%, 80%, 85%, 90%, 95%, or 99% diastereomerically pure.
12. A chirally controlled oligonucleotide composition of an oligonucleotide of any one of embodiments 1-10.
13. The composition of embodiment 11, wherein at least about 10%, 20%, 30%, 40%, 50%, 55%, 60%, 65%, 70%, 75%, 80%, 85%, 90%, 95% r 99% of the oligonucleotides in the composition, or the oligonucleotides in the composition that share the same base sequence as the oligonucleotide, are each independently an oligonucleotide of any one of embodiments 1-10.
14. A pharmaceutical composition comprising a therapeutically effective amount of an oligonucleotide and a pharmaceutically acceptable inactive ingredient, wherein the oligonucleotide is an oligonucleotide of any one of embodiments 1-11.
15. The composition of embodiment 14, wherein at least about 10%, 20%, 30%, 40%, 50%, 55%, 60%, 65%, 70%, 75%, 80%, 85%, 90%, 95% r 99% of the oligonucleotides in the composition, or the oligonucleotides in the composition that share the same base sequence as the oligonucleotide, are each independently an oligonucleotide of any one of embodiments 1-10.
16. The composition of any one of embodiments 12-15, wherein the oligonucleotide is in the form of a pharmaceutically acceptable salt.
17. The composition of any one of embodiments 12-15, wherein the oligonucleotide is in a sodium salt form.
18. A composition comprising an oligonucleotide selected from WV-21404, WV-21405, WV-21406, WV-21412, WV-10786, WV-10787, WV-10790, WV-10791, WV-10806, WV-10810, WV-10811, WV-12282, WV-12283, WV-12284, WV-14914, WV-15078, WV-15080, WV-17782, WV-19824, WV-19825, WV-19840, WV-19841, WV-21178, WV-21179, WV-21180, WV-21181, WV-21267, WV-21271, WV-21274, WV-21403, WV-21409, WV-21410, WV-21447, WV-21448, WV-23689, WV-23690, WV-23691, WV-23692, WV-28152, WV-28153, WV-28154, WV-28155, WV-28156, WV-28157, WV-28158, WV-28159, WV-28160, WV-28161, WV-28162, WV-28163, WV-28164, WV-28165, WV-28166, WV-28167, WV-28168, and WV-9679.
19. The composition of embodiment 18, wherein the oligonucleotide is in the form of a pharmaceutically acceptable salt.
20. A method of treating, preventing, delaying onset of, and/or decreasing the severity of at least one symptom of Huntington's Disease, wherein the method comprises administering to a subject suffering therefrom or susceptible thereto an effective amount of an oligonucleotide or composition of any one of the preceding embodiments.
21. The method of embodiment 20, wherein the subject has a HTT allele that comprises an expanded CAG repeat region and is fully complementary to the base sequence of the oligonucleotide.
22. An oligonucleotide, composition or method described in the present application.

1. An oligonucleotide, wherein:
(a) the oligonucleotide targets SNP rs362273, and the base sequence of the oligonucleotide comprises at least 15 contiguous bases, including the SNP position, of the base sequence GTTGATCTGTAGCAGCAGCT, wherein each T can be independently replaced with U;
(b) the oligonucleotide targets SNP rs362272, and the base sequence of the oligonucleotide comprises at least 15 contiguous bases, including the SNP position, of the base sequence ACATAGAGGACGCCGTGCAG, AGAGGACGCCGTGCAGGGCT, ATAGAGGACGCCGTGCAGGG, CACATAGAGGACGCCGTGCA, CATAGAGGACGCCGTGCAGG, GCACATAGAGGACGCCGTGC, or TAGAGGACGCCGTGCAGGGC, wherein each T can be independently replaced with U;
(c) the oligonucleotide targets SNP rs362273, and the base sequence of the oligonucleotide comprises at least 15 contiguous bases, including the SNP position, of the base sequence AGCTGCTGCTACAGATCAAC, AGCTGCTGCTGCAGATCAAC, GGTTGATCTGTAGCAGCAGCT, GTTGATCTGTAGCAGCAGCT, or TTGATCTGTAGCAGCAGCT, wherein each T can be independently replaced with U;
(d) the oligonucleotide targets SNP rs362307, and the base sequence of the oligonucleotide comprises at least 15 contiguous bases, including the SNP position, of the base sequence GGCACAAGGGCACAGAC, GGCACAAGGGCACAGACT, or GGCACAAGGGCACAGACTT, wherein each T can be independently replaced with U;
(e) the oligonucleotide targets SNP rs362331, and the base sequence of the oligonucleotide comprises at least 15 contiguous bases, including the SNP position, of the base sequence GTGCACACAGTAGATGAGGG, wherein each T can be independently replaced with U; or
(f) the oligonucleotide targets SNP rs363099, and the base sequence of the oligonucleotide comprises at least 15 contiguous bases, including the SNP position, of the base sequence AAGGCTGAGCGGAGAAACCC, AGGCTGAGCGGAGAAACCCT, CAAGGCTGAGCGGAGAAACC, CTGAGCGGAGAAACCCTCCA, GCTGAGCGGAGAAACCCTCC, GGCTGAGCGGAGAAACCCTC, or TGAGCGGAGAAACCCTCCAA, wherein each T can be independently replaced with U; and
wherein the oligonucleotide comprises one or more chiral internucleotidic linkages.

2. The oligonucleotide of claim 1, wherein the base sequence of the oligonucleotide comprises or is:
(a) GTTGATCTGTAGCAGCAGCT, wherein each T can be independently replaced with U;
(b) ACATAGAGGACGCCGTGCAG, AGAGGACGCCGTGCAGGGCT, ATAGAGGACGCCGTGCAGGG, CACATAGAGGACGCCGTGCA, CATAGAGGACGCCGTGCAGG, GCACATAGAGGACGCCGTGC, or TAGAGGACGCCGTGCAGGGC, wherein each T can be independently replaced with U;
(c) AGCTGCTGCTACAGATCAAC, AGCTGCTGCTGCAGATCAAC, GGTTGATCTGTAGCAGCAGCT, GTTGATCTGTAGCAGCAGCT, or TTGATCTGTAGCAGCAGCT, wherein each T can be independently replaced with U;
(d) GGCACAAGGGCACAGAC, GGCACAAGGGCACAGACT, or GGCACAAGGGCACAGACTT, wherein each T can be independently replaced with U;
(e) GTGCACACAGTAGATGAGGG, wherein each T can be independently replaced with U; or
(f) AAGGCTGAGCGGAGAAACCC, AGGCTGAGCGGAGAAACCCT, CAAGGCTGAGCGGAGAAACC, CTGAGCGGAGAAACCCTCCA, GCTGAGCGGAGAAACCCTCC, GGCTGAGCGGAGAAACCCTC, or TGAGCGGAGAAACCCTCCAA, wherein each T can be independently replaced with U.

3. The oligonucleotide of claim 1 or 2, wherein each internucleotidic linkage of the oligonucleotide is independently a natural phosphate linkage, a phosphorothioate linkage, or a 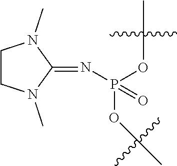 [SMILES: *OP(=O)(N=C1N(C)CCN1C)O*]
(n001) linkage.

4. The oligonucleotide of claim 1 or 2, wherein the oligonucleotide comprises one or more natural phosphate linkages, one or more Sp phosphorothioate linkages, and one or more Rp n001 linkages.

5. The oligonucleotide of any one of claims 1-4, wherein the oligonucleotide comprises or consists of: a 5′-wing and a 3′-wing, each of which independently comprises one or more modified sugars, and a core between the 5′-wing and the 3′-wing.

6. The oligonucleotide of claim 5, wherein the oligonucleotide comprises a 5′-wing comprising 5 consecutive 2′-OMe modified sugars and a 3′-wing comprising 5 consecutive 2′-OMe modified sugars.

7. The oligonucleotide of any one of claims 5-6, wherein the core comprises one or more unmodified natural DNA sugars.

8. An oligonucleotide, wherein the oligonucleotide is WV-21404, WV-21405, WV-21406, WV-21412, WV-12282, WV-12283, WV-12284, WV-19840, WV-21178, WV-21179, WV-21180, WV-21181, WV-21403, WV-21409, WV-21410, WV-21447, WV-21448, WV-23689, WV-23690, WV-23691, WV-23692, WV-28152, WV-28153, WV-28154, WV-28155, WV-28157, WV-28158, WV-28159, WV-28160, WV-28161, WV-28162, WV-28163, WV-28164, WV-28165, WV-28166, WV-28167, or WV-28168.

9. The oligonucleotide of any one of the preceding claims, wherein the oligonucleotide is in the form of a pharmaceutically acceptable salt.

10. The oligonucleotide of any one of the preceding claims, wherein the oligonucleotide is in a sodium salt form.

11. The oligonucleotide of any one of the preceding claims, wherein the oligonucleotide is at least about 10%, 20%, 30%, 40%, 50%, 55%, 60%, 65%, 70%, 75%, 80%, 85%, 90%, 95%, or 99% diastereomerically pure.

12. A chirally controlled oligonucleotide composition of an oligonucleotide of any one of claims 1-10.

13. The composition of claim 11, wherein at least about 10%, 20%, 30%, 40%, 50%, 55%, 60%, 65%, 70%, 75%, 80%, 85%, 90%, 95% r 99% of the oligonucleotides in the composition, or the oligonucleotides in the composition that share the same base sequence as the oligonucleotide, are each independently an oligonucleotide of any one of claims 1-10.

14. A pharmaceutical composition comprising a therapeutically effective amount of an oligonucleotide and a pharmaceutically acceptable inactive ingredient, wherein the oligonucleotide is an oligonucleotide of any one of claims 1-11.

15. The composition of claim 14, wherein at least about 10%, 20%, 30%, 40%, 50%, 55%, 60%, 65%, 70%, 75%, 80%, 85%, 90%, 95% r 99% of the oligonucleotides in the composition, or the oligonucleotides in the composition that share the same base sequence as the oligonucleotide, are each independently an oligonucleotide of any one of claims 1-10.

16. The composition of any one of claims 12-15, wherein the oligonucleotide is in the form of a pharmaceutically acceptable salt.

17. The composition of any one of claims 12-15, wherein the oligonucleotide is in a sodium salt form.

18. A composition comprising an oligonucleotide selected from WV-21404, WV-21405, WV-21406, WV-21412, WV-10786, WV-10787, WV-10790, WV-10791, WV-10806, WV-10810, WV-10811, WV-12282, WV-12283, WV-12284, WV-14914, WV-15078, WV-15080, WV-17782, WV-19824, WV-19825, WV-19840, WV-19841, WV-21178, WV-21179, WV-21180, WV-21181, WV-21267, WV-21271, WV-21274, WV-21403, WV-21409, WV-21410, WV-21447, WV-21448, WV-23689, WV-23690, WV-23691, WV-23692, WV-28152, WV-28153, WV-28154, WV-28155, WV-28156, WV-28157, WV-28158, WV-28159, WV-28160, WV-28161, WV-28162, WV-28163, WV-28164, WV-28165, WV-28166, WV-28167, WV-28168, and WV-9679.

19. The composition of claim 18, wherein the oligonucleotide is in the form of a pharmaceutically acceptable salt.

20. A method of treating, preventing, delaying onset of, and/or decreasing the severity of at least one symptom of Huntington's Disease, wherein the method comprises administering to a subject suffering therefrom or susceptible thereto an effective amount of an oligonucleotide or composition of any one of the preceding claims.

21. The method of claim 20, wherein the subject has a HTT allele that comprises an expanded CAG repeat region and is fully complementary to the base sequence of the oligonucleotide.

22. An oligonucleotide, composition or method described in the present application.


SEQUENCE LISTING (2358 sequences)

SEQ ID NO: 1 - DNA, 20 bp, Artificial Sequence
  Synthetic
acatagaggacgccgtgcag

SEQ ID NO: 2 - DNA, 20 bp, Artificial Sequence
  Synthetic
agaggacgccgtgcagggct

SEQ ID NO: 3 - DNA, 20 bp, Artificial Sequence
  Synthetic
atagaggacgccgtgcaggg

SEQ ID NO: 4 - DNA, 20 bp, Artificial Sequence
  Synthetic
cacatagaggacgccgtgca

SEQ ID NO: 5 - DNA, 20 bp, Artificial Sequence
  Synthetic
catagaggacgccgtgcagg

SEQ ID NO: 6 - DNA, 20 bp, Artificial Sequence
  Synthetic
gcacatagaggacgccgtgc

SEQ ID NO: 7 - DNA, 20 bp, Artificial Sequence
  Synthetic
tagaggacgccgtgcagggc

SEQ ID NO: 8 - DNA, 20 bp, Artificial Sequence
  Synthetic
agctgctgctacagatcaac

SEQ ID NO: 9 - DNA, 20 bp, Artificial Sequence
  Synthetic
agctgctgctgcagatcaac

SEQ ID NO: 10 - DNA, 21 bp, Artificial Sequence
  Synthetic
ggttgatctgtagcagcagct

SEQ ID NO: 11 - DNA, 20 bp, Artificial Sequence
  Synthetic
gttgatctgtagcagcagct

SEQ ID NO: 12 - DNA, 20 bp, Artificial Sequence
  Synthetic
gttgatctgtagcagcagct

SEQ ID NO: 13 - DNA, 19 bp, Artificial Sequence
  Synthetic
ttgatctgtagcagcagct

SEQ ID NO: 14 - DNA, 20 bp, Artificial Sequence
  Synthetic
gttgatctgtagcagcagct

SEQ ID NO: 15 - DNA, 20 bp, Artificial Sequence
  Synthetic
cacaagggcacagacttcca

SEQ ID NO: 16 - DNA, 17 bp, Artificial Sequence
  Synthetic
ggcacaagggcacagac

SEQ ID NO: 17 - DNA, 18 bp, Artificial Sequence
  Synthetic
ggcacaagggcacagact

SEQ ID NO: 18 - DNA, 19 bp, Artificial Sequence
  Synthetic
ggcacaagggcacagactt

SEQ ID NO: 19 - DNA, 20 bp, Artificial Sequence
  Synthetic
ggcacaagggcacagacttc

SEQ ID NO: 20 - DNA, 20 bp, Artificial Sequence
  Synthetic
agtgcacacagtagatgagg

SEQ ID NO: 21 - DNA, 20 bp, Artificial Sequence
  Synthetic
gtgcacacagtagatgaggg

SEQ ID NO: 22 - DNA, 20 bp, Artificial Sequence
  Synthetic
tgcacacagtagatgaggga

SEQ ID NO: 23 - DNA, 20 bp, Artificial Sequence
  Synthetic
aaggctgagcggagaaaccc

SEQ ID NO: 24 - DNA, 20 bp, Artificial Sequence
  Synthetic
aggctgagcggagaaaccct

SEQ ID NO: 25 - DNA, 20 bp, Artificial Sequence
  Synthetic
caaggctgagcggagaaacc

SEQ ID NO: 26 - DNA, 20 bp, Artificial Sequence
  Synthetic
ctgagcggagaaaccctcca

SEQ ID NO: 27 - DNA, 20 bp, Artificial Sequence
  Synthetic
gctgagcggagaaaccctcc

SEQ ID NO: 28 - DNA, 20 bp, Artificial Sequence
  Synthetic
ggctgagcggagaaaccctc

SEQ ID NO: 29 - DNA, 20 bp, Artificial Sequence
  Synthetic
tgagcggagaaaccctccaa

SEQ ID NO: 30 - DNA, 20 bp, Artificial Sequence
  Synthetic
accgccatccccgccgtagc

SEQ ID NO: 31 - DNA, 20 bp, Artificial Sequence
  Synthetic
ccgccatccccgccgtagcc

SEQ ID NO: 32 - DNA, 20 bp, Artificial Sequence
  Synthetic
cgccatccccgccgtagcct

SEQ ID NO: 33 - DNA, 20 bp, Artificial Sequence
  Synthetic
ctcagtaacattgacaccac

SEQ ID NO: 34 - DNA, 20 bp, Artificial Sequence
  Synthetic
gccatccccgccgtagcctg

SEQ ID NO: 35 - DNA, 20 bp, Artificial Sequence
  Synthetic
ggctctgggttgctgggtca

SEQ ID NO: 36 - DNA, 20 bp, Artificial Sequence
  Synthetic
ggtgtccctcatgggctctg

SEQ ID NO: 37 - DNA, 20 bp, Artificial Sequence
  Synthetic
gttaccgccatccccgccgt

SEQ ID NO: 38 - DNA, 20 bp, Artificial Sequence
  Synthetic
tctccattctatcttatgtt

SEQ ID NO: 39 - DNA, 20 bp, Artificial Sequence
  Synthetic
gttgatctgtagtagcagct

SEQ ID NO: 40 - DNA, 21 bp, Artificial Sequence
  Synthetic
gtgcaacacagtagatgaggg

SEQ ID NO: 41 - DNA, 21 bp, Artificial Sequence
  Synthetic
ggcacaaagggcacagacttc

SEQ ID NO: 42 - DNA, 16 bp, Artificial Sequence
  Synthetic
caagggcacagacttc

SEQ ID NO: 43 - DNA, 15 bp, Artificial Sequence
  Synthetic
aagggcacagacttc

SEQ ID NO: 44 - RNA, 21 nt, Artificial Sequence
  Synthetic
gcauagcgagcgagggaaaac

SEQ ID NO: 45 - RNA, 21 nt, Artificial Sequence
  Synthetic
guuuucccucgcucgcuaugc

SEQ ID NO: 46 - RNA, 20 nt, Artificial Sequence
  Synthetic
gaagucugugcccuugugcc

SEQ ID NO: 47 - DNA, 20 bp, Artificial Sequence
  Synthetic
gugcacacagtagatgaggg

SEQ ID NO: 48 - RNA, 16 nt, Artificial Sequence
  Synthetic
caagggcacagacuuc

SEQ ID NO: 49 - DNA, 12 bp, Artificial Sequence
  Synthetic
ggcacaagggca

SEQ ID NO: 50 - DNA, 16 bp, Artificial Sequence
  Synthetic
gugcacacagtagatg

SEQ ID NO: 51 - DNA, 12 bp, Artificial Sequence
  Synthetic
gugcacacagta

SEQ ID NO: 52 - RNA, 20 nt, Artificial Sequence
  Synthetic
ggcacaagggcacagacuuc

SEQ ID NO: 53 - DNA, 20 bp, Artificial Sequence
  Synthetic
gugcacacagtagatgaggg

SEQ ID NO: 54 - DNA, 21 bp, Artificial Sequence
  Synthetic
tacagacuuccaaaggcuctu

SEQ ID NO: 55 - DNA, 21 bp, Artificial Sequence
  Synthetic
tcacagacuuccaaaggcutu

SEQ ID NO: 56 - DNA, 21 bp, Artificial Sequence
  Synthetic
tgcacagacuuccaaaggctu

SEQ ID NO: 57 - DNA, 21 bp, Artificial Sequence
  Synthetic
tggcacagacuuccaaaggtu

SEQ ID NO: 58 - DNA, 21 bp, Artificial Sequence
  Synthetic
tgggcacagacuuccaaagtu

SEQ ID NO: 59 - DNA, 21 bp, Artificial Sequence
  Synthetic
tagggcacagacuuccaaatu

SEQ ID NO: 60 - DNA, 21 bp, Artificial Sequence
  Synthetic
taagggcacagacuuccaatu

SEQ ID NO: 61 - DNA, 21 bp, Artificial Sequence
  Synthetic
tcaagggcacagacuuccatu

SEQ ID NO: 62 - DNA, 21 bp, Artificial Sequence
  Synthetic
tacaagggcacagacuucctu

SEQ ID NO: 63 - DNA, 21 bp, Artificial Sequence
  Synthetic
tcacaagggcacagacuuctu

SEQ ID NO: 64 - DNA, 21 bp, Artificial Sequence
  Synthetic
tgcacaagggcacagacuutu

SEQ ID NO: 65 - DNA, 21 bp, Artificial Sequence
  Synthetic
tggcacaagggcacagacutu

SEQ ID NO: 66 - DNA, 21 bp, Artificial Sequence
  Synthetic
tgggcacaagggcacagactu

SEQ ID NO: 67 - DNA, 21 bp, Artificial Sequence
  Synthetic
tagggcacaagggcacagatu

SEQ ID NO: 68 - DNA, 21 bp, Artificial Sequence
  Synthetic
tcagggcacaagggcacagtu

SEQ ID NO: 69 - DNA, 21 bp, Artificial Sequence
  Synthetic
tgcagggcacaagggcacatu

SEQ ID NO: 70 - DNA, 21 bp, Artificial Sequence
  Synthetic
tggcagggcacaagggcactu

SEQ ID NO: 71 - DNA, 21 bp, Artificial Sequence
  Synthetic
taggcagggcacaagggcatu

SEQ ID NO: 72 - DNA, 21 bp, Artificial Sequence
  Synthetic
tacagacuuccaaaggcuctu

SEQ ID NO: 73 - DNA, 21 bp, Artificial Sequence
  Synthetic
tcacagacuuccaaaggcutu

SEQ ID NO: 74 - DNA, 21 bp, Artificial Sequence
  Synthetic
tgcacagacuuccaaaggctu

SEQ ID NO: 75 - DNA, 21 bp, Artificial Sequence
  Synthetic
tggcacagacuuccaaaggtu

SEQ ID NO: 76 - DNA, 21 bp, Artificial Sequence
  Synthetic
tgggcacagacuuccaaagtu

SEQ ID NO: 77 - DNA, 21 bp, Artificial Sequence
  Synthetic
tagggcacagacuuccaaatu

SEQ ID NO: 78 - DNA, 21 bp, Artificial Sequence
  Synthetic
taagggcacagacuuccaatu

SEQ ID NO: 79 - DNA, 21 bp, Artificial Sequence
  Synthetic
tcaagggcacagacuuccatu

SEQ ID NO: 80 - DNA, 21 bp, Artificial Sequence
  Synthetic
tacaagggcacagacuucctu

SEQ ID NO: 81 - DNA, 21 bp, Artificial Sequence
  Synthetic
tcacaagggcacagacuuctu

SEQ ID NO: 82 - DNA, 21 bp, Artificial Sequence
  Synthetic
tgcacaagggcacagacuutu

SEQ ID NO: 83 - DNA, 21 bp, Artificial Sequence
  Synthetic
tggcacaagggcacagacutu

SEQ ID NO: 84 - DNA, 21 bp, Artificial Sequence
  Synthetic
tgggcacaagggcacagactu

SEQ ID NO: 85 - DNA, 21 bp, Artificial Sequence
  Synthetic
tagggcacaagggcacagatu

SEQ ID NO: 86 - DNA, 21 bp, Artificial Sequence
  Synthetic
tcagggcacaagggcacagtu

SEQ ID NO: 87 - DNA, 21 bp, Artificial Sequence
  Synthetic
tgcagggcacaagggcacatu

SEQ ID NO: 88 - DNA, 21 bp, Artificial Sequence
  Synthetic
tggcagggcacaagggcactu

SEQ ID NO: 89 - DNA, 21 bp, Artificial Sequence
  Synthetic
taggcagggcacaagggcatu

SEQ ID NO: 90 - DNA, 23 bp, Artificial Sequence
  Synthetic
tacagacuuccaaaggcuccgtu

SEQ ID NO: 91 - DNA, 23 bp, Artificial Sequence
  Synthetic
tcacagacuuccaaaggcucctu

SEQ ID NO: 92 - DNA, 23 bp, Artificial Sequence
  Synthetic
tgcacagacuuccaaaggcuctu

SEQ ID NO: 93 - DNA, 23 bp, Artificial Sequence
  Synthetic
tggcacagacuuccaaaggcutu

SEQ ID NO: 94 - DNA, 23 bp, Artificial Sequence
  Synthetic
tgggcacagacuuccaaaggctu

SEQ ID NO: 95 - DNA, 23 bp, Artificial Sequence
  Synthetic
tagggcacagacuuccaaaggtu

SEQ ID NO: 96 - DNA, 23 bp, Artificial Sequence
  Synthetic
taagggcacagacuuccaaagtu

SEQ ID NO: 97 - DNA, 23 bp, Artificial Sequence
  Synthetic
tcaagggcacagacuuccaaatu

SEQ ID NO: 98 - DNA, 23 bp, Artificial Sequence
  Synthetic
tacaagggcacagacuuccaatu

SEQ ID NO: 99 - DNA, 23 bp, Artificial Sequence
  Synthetic
tcacaagggcacagacuuccatu

SEQ ID NO: 100 - DNA, 23 bp, Artificial Sequence
  Synthetic
tgcacaagggcacagacuucctu

SEQ ID NO: 101 - DNA, 23 bp, Artificial Sequence
  Synthetic
tggcacaagggcacagacuuctu

SEQ ID NO: 102 - DNA, 23 bp, Artificial Sequence
  Synthetic
tgggcacaagggcacagacuutu

SEQ ID NO: 103 - DNA, 23 bp, Artificial Sequence
  Synthetic
tagggcacaagggcacagacutu

SEQ ID NO: 104 - DNA, 23 bp, Artificial Sequence
  Synthetic
tcagggcacaagggcacagactu

SEQ ID NO: 105 - DNA, 23 bp, Artificial Sequence
  Synthetic
tgcagggcacaagggcacagatu

SEQ ID NO: 106 - DNA, 23 bp, Artificial Sequence
  Synthetic
tggcagggcacaagggcacagtu

SEQ ID NO: 107 - DNA, 23 bp, Artificial Sequence
  Synthetic
taggcagggcacaagggcacatu

SEQ ID NO: 108 - DNA, 23 bp, Artificial Sequence
  Synthetic
tgaggcagggcacaagggcactu

SEQ ID NO: 109 - DNA, 23 bp, Artificial Sequence
  Synthetic
tggaggcagggcacaagggcatu

SEQ ID NO: 110 - DNA, 23 bp, Artificial Sequence
  Synthetic
tacagacuuccaaaggcuccgtu

SEQ ID NO: 111 - DNA, 23 bp, Artificial Sequence
  Synthetic
tcacagacuuccaaaggcucctu

SEQ ID NO: 112 - DNA, 23 bp, Artificial Sequence
  Synthetic
tgcacagacuuccaaaggcuctu

SEQ ID NO: 113 - DNA, 23 bp, Artificial Sequence
  Synthetic
tggcacagacuuccaaaggcutu

SEQ ID NO: 114 - DNA, 23 bp, Artificial Sequence
  Synthetic
tgggcacagacuuccaaaggctu

SEQ ID NO: 115 - DNA, 23 bp, Artificial Sequence
  Synthetic
tagggcacagacuuccaaaggtu

SEQ ID NO: 116 - DNA, 23 bp, Artificial Sequence
  Synthetic
taagggcacagacuuccaaagtu

SEQ ID NO: 117 - DNA, 23 bp, Artificial Sequence
  Synthetic
tcaagggcacagacuuccaaatu

SEQ ID NO: 118 - DNA, 23 bp, Artificial Sequence
  Synthetic
tacaagggcacagacuuccaatu

SEQ ID NO: 119 - DNA, 23 bp, Artificial Sequence
  Synthetic
tcacaagggcacagacuuccatu

SEQ ID NO: 120 - DNA, 23 bp, Artificial Sequence
  Synthetic
tgcacaagggcacagacuucctu

SEQ ID NO: 121 - DNA, 23 bp, Artificial Sequence
  Synthetic
tggcacaagggcacagacuuctu

SEQ ID NO: 122 - DNA, 23 bp, Artificial Sequence
  Synthetic
tgggcacaagggcacagacuutu

SEQ ID NO: 123 - DNA, 23 bp, Artificial Sequence
  Synthetic
tagggcacaagggcacagacutu

SEQ ID NO: 124 - DNA, 23 bp, Artificial Sequence
  Synthetic
tcagggcacaagggcacagactu

SEQ ID NO: 125 - DNA, 23 bp, Artificial Sequence
  Synthetic
tgcagggcacaagggcacagatu

SEQ ID NO: 126 - DNA, 23 bp, Artificial Sequence
  Synthetic
tggcagggcacaagggcacagtu

SEQ ID NO: 127 - DNA, 23 bp, Artificial Sequence
  Synthetic
taggcagggcacaagggcacatu

SEQ ID NO: 128 - DNA, 23 bp, Artificial Sequence
  Synthetic
tgaggcagggcacaagggcactu

SEQ ID NO: 129 - DNA, 23 bp, Artificial Sequence
  Synthetic
tggaggcagggcacaagggcatu

SEQ ID NO: 130 - DNA, 21 bp, Artificial Sequence
  Synthetic
tagaugagggagcaggcgutu

SEQ ID NO: 131 - DNA, 21 bp, Artificial Sequence
  Synthetic
tuagaugagggagcaggcgtu

SEQ ID NO: 132 - DNA, 21 bp, Artificial Sequence
  Synthetic
tguagaugagggagcaggctu

SEQ ID NO: 133 - DNA, 21 bp, Artificial Sequence
  Synthetic
taguagaugagggagcaggtu

SEQ ID NO: 134 - DNA, 21 bp, Artificial Sequence
  Synthetic
tcaguagaugagggagcagtu

SEQ ID NO: 135 - DNA, 21 bp, Artificial Sequence
  Synthetic
tacaguagaugagggagcatu

SEQ ID NO: 136 - DNA, 21 bp, Artificial Sequence
  Synthetic
tcacaguagaugagggagctu

SEQ ID NO: 137 - DNA, 21 bp, Artificial Sequence
  Synthetic
tacacaguagaugagggagtu

SEQ ID NO: 138 - DNA, 21 bp, Artificial Sequence
  Synthetic
tcacacaguagaugagggatu

SEQ ID NO: 139 - DNA, 21 bp, Artificial Sequence
  Synthetic
tgcacacaguagaugagggtu

SEQ ID NO: 140 - DNA, 21 bp, Artificial Sequence
  Synthetic
tugcacacaguagaugaggtu

SEQ ID NO: 141 - DNA, 21 bp, Artificial Sequence
  Synthetic
tgugcacacaguagaugagtu

SEQ ID NO: 142 - DNA, 21 bp, Artificial Sequence
  Synthetic
tagugcacacaguagaugatu

SEQ ID NO: 143 - DNA, 21 bp, Artificial Sequence
  Synthetic
taagugcacacaguagaugtu

SEQ ID NO: 144 - DNA, 21 bp, Artificial Sequence
  Synthetic
tgaagugcacacaguagautu

SEQ ID NO: 145 - DNA, 21 bp, Artificial Sequence
  Synthetic
tugaagugcacacaguagatu

SEQ ID NO: 146 - DNA, 21 bp, Artificial Sequence
  Synthetic
taugaagugcacacaguagtu

SEQ ID NO: 147 - DNA, 21 bp, Artificial Sequence
  Synthetic
tgaugaagugcacacaguatu

SEQ ID NO: 148 - DNA, 21 bp, Artificial Sequence
  Synthetic
tagaugagggagcaggcgutu

SEQ ID NO: 149 - DNA, 21 bp, Artificial Sequence
  Synthetic
tuagaugagggagcaggcgtu

SEQ ID NO: 150 - DNA, 21 bp, Artificial Sequence
  Synthetic
tguagaugagggagcaggctu

SEQ ID NO: 151 - DNA, 21 bp, Artificial Sequence
  Synthetic
taguagaugagggagcaggtu

SEQ ID NO: 152 - DNA, 21 bp, Artificial Sequence
  Synthetic
tcaguagaugagggagcagtu

SEQ ID NO: 153 - DNA, 21 bp, Artificial Sequence
  Synthetic
tacaguagaugagggagcatu

SEQ ID NO: 154 - DNA, 21 bp, Artificial Sequence
  Synthetic
tcacaguagaugagggagctu

SEQ ID NO: 155 - DNA, 21 bp, Artificial Sequence
  Synthetic
tacacaguagaugagggagtu

SEQ ID NO: 156 - DNA, 21 bp, Artificial Sequence
  Synthetic
tcacacaguagaugagggatu

SEQ ID NO: 157 - DNA, 21 bp, Artificial Sequence
  Synthetic
tgcacacaguagaugagggtu

SEQ ID NO: 158 - DNA, 21 bp, Artificial Sequence
  Synthetic
tugcacacaguagaugaggtu

SEQ ID NO: 159 - DNA, 21 bp, Artificial Sequence
  Synthetic
tgugcacacaguagaugagtu

SEQ ID NO: 160 - DNA, 21 bp, Artificial Sequence
  Synthetic
tagugcacacaguagaugatu

SEQ ID NO: 161 - DNA, 21 bp, Artificial Sequence
  Synthetic
taagugcacacaguagaugtu

SEQ ID NO: 162 - DNA, 21 bp, Artificial Sequence
  Synthetic
tgaagugcacacaguagautu

SEQ ID NO: 163 - DNA, 21 bp, Artificial Sequence
  Synthetic
tugaagugcacacaguagatu

SEQ ID NO: 164 - DNA, 21 bp, Artificial Sequence
  Synthetic
taugaagugcacacaguagtu

SEQ ID NO: 165 - DNA, 21 bp, Artificial Sequence
  Synthetic
tgaugaagugcacacaguatu

SEQ ID NO: 166 - DNA, 23 bp, Artificial Sequence
  Synthetic
tagaugagggagcaggcguggtu

SEQ ID NO: 167 - DNA, 23 bp, Artificial Sequence
  Synthetic
tuagaugagggagcaggcgugtu

SEQ ID NO: 168 - DNA, 23 bp, Artificial Sequence
  Synthetic
tguagaugagggagcaggcgutu

SEQ ID NO: 169 - DNA, 23 bp, Artificial Sequence
  Synthetic
taguagaugagggagcaggcgtu

SEQ ID NO: 170 - DNA, 23 bp, Artificial Sequence
  Synthetic
tcaguagaugagggagcaggctu

SEQ ID NO: 171 - DNA, 23 bp, Artificial Sequence
  Synthetic
tacaguagaugagggagcaggtu

SEQ ID NO: 172 - DNA, 23 bp, Artificial Sequence
  Synthetic
tcacaguagaugagggagcagtu

SEQ ID NO: 173 - DNA, 23 bp, Artificial Sequence
  Synthetic
tacacaguagaugagggagcatu

SEQ ID NO: 174 - DNA, 23 bp, Artificial Sequence
  Synthetic
tcacacaguagaugagggagctu

SEQ ID NO: 175 - DNA, 23 bp, Artificial Sequence
  Synthetic
tgcacacaguagaugagggagtu

SEQ ID NO: 176 - DNA, 23 bp, Artificial Sequence
  Synthetic
tugcacacaguagaugagggatu

SEQ ID NO: 177 - DNA, 23 bp, Artificial Sequence
  Synthetic
tgugcacacaguagaugagggtu

SEQ ID NO: 178 - DNA, 23 bp, Artificial Sequence
  Synthetic
tagugcacacaguagaugaggtu

SEQ ID NO: 179 - DNA, 23 bp, Artificial Sequence
  Synthetic
taagugcacacaguagaugagtu

SEQ ID NO: 180 - DNA, 23 bp, Artificial Sequence
  Synthetic
tgaagugcacacaguagaugatu

SEQ ID NO: 181 - DNA, 23 bp, Artificial Sequence
  Synthetic
tugaagugcacacaguagaugtu

SEQ ID NO: 182 - DNA, 23 bp, Artificial Sequence
  Synthetic
taugaagugcacacaguagautu

SEQ ID NO: 183 - DNA, 23 bp, Artificial Sequence
  Synthetic
tgaugaagugcacacaguagatu

SEQ ID NO: 184 - DNA, 23 bp, Artificial Sequence
  Synthetic
tggaugaagugcacacaguagtu

SEQ ID NO: 185 - DNA, 23 bp, Artificial Sequence
  Synthetic
taggaugaagugcacacaguatu

SEQ ID NO: 186 - DNA, 23 bp, Artificial Sequence
  Synthetic
tagaugagggagcaggcguggtu

SEQ ID NO: 187 - DNA, 23 bp, Artificial Sequence
  Synthetic
tuagaugagggagcaggcgugtu

SEQ ID NO: 188 - DNA, 23 bp, Artificial Sequence
  Synthetic
tguagaugagggagcaggcgutu

SEQ ID NO: 189 - DNA, 23 bp, Artificial Sequence
  Synthetic
taguagaugagggagcaggcgtu

SEQ ID NO: 190 - DNA, 23 bp, Artificial Sequence
  Synthetic
tcaguagaugagggagcaggctu

SEQ ID NO: 191 - DNA, 23 bp, Artificial Sequence
  Synthetic
tacaguagaugagggagcaggtu

SEQ ID NO: 192 - DNA, 23 bp, Artificial Sequence
  Synthetic
tcacaguagaugagggagcagtu

SEQ ID NO: 193 - DNA, 23 bp, Artificial Sequence
  Synthetic
tacacaguagaugagggagcatu

SEQ ID NO: 194 - DNA, 23 bp, Artificial Sequence
  Synthetic
tcacacaguagaugagggagctu

SEQ ID NO: 195 - DNA, 23 bp, Artificial Sequence
  Synthetic
tgcacacaguagaugagggagtu

SEQ ID NO: 196 - DNA, 23 bp, Artificial Sequence
  Synthetic
tugcacacaguagaugagggatu

SEQ ID NO: 197 - DNA, 23 bp, Artificial Sequence
  Synthetic
tgugcacacaguagaugagggtu

SEQ ID NO: 198 - DNA, 23 bp, Artificial Sequence
  Synthetic
tagugcacacaguagaugaggtu

SEQ ID NO: 199 - DNA, 23 bp, Artificial Sequence
  Synthetic
taagugcacacaguagaugagtu

SEQ ID NO: 200 - DNA, 23 bp, Artificial Sequence
  Synthetic
tgaagugcacacaguagaugatu

SEQ ID NO: 201 - DNA, 23 bp, Artificial Sequence
  Synthetic
tugaagugcacacaguagaugtu

SEQ ID NO: 202 - DNA, 23 bp, Artificial Sequence
  Synthetic
taugaagugcacacaguagautu

SEQ ID NO: 203 - DNA, 23 bp, Artificial Sequence
  Synthetic
tgaugaagugcacacaguagatu

SEQ ID NO: 204 - DNA, 23 bp, Artificial Sequence
  Synthetic
tggaugaagugcacacaguagtu

SEQ ID NO: 205 - DNA, 23 bp, Artificial Sequence
  Synthetic
taggaugaagugcacacaguatu

SEQ ID NO: 206 - RNA, 19 nt, Artificial Sequence
  Synthetic
gcacaagggcacagacuuc

SEQ ID NO: 207 - RNA, 18 nt, Artificial Sequence
  Synthetic
cacaagggcacagacuuc

SEQ ID NO: 208 - DNA, 19 bp, Artificial Sequence
  Synthetic
ugcacacagtagatgaggg

SEQ ID NO: 209 - DNA, 18 bp, Artificial Sequence
  Synthetic
gcacacagtagatgaggg

SEQ ID NO: 210 - RNA, 20 nt, Artificial Sequence
  Synthetic
ggcacaagggcacagacuuc

SEQ ID NO: 211 - RNA, 20 nt, Artificial Sequence
  Synthetic
ggcacaagggcacagacuuc

SEQ ID NO: 212 - RNA, 20 nt, Artificial Sequence
  Synthetic
ggcacaagggcacagacuuc

SEQ ID NO: 213 - RNA, 20 nt, Artificial Sequence
  Synthetic
ggcacaagggcacagacuuc

SEQ ID NO: 214 - RNA, 20 nt, Artificial Sequence
  Synthetic
ggcacaagggcacagacuuc

SEQ ID NO: 215 - DNA, 20 bp, Artificial Sequence
  Synthetic
gtgcacacagtagatgaggg

SEQ ID NO: 216 - DNA, 20 bp, Artificial Sequence
  Synthetic
gtgcacacagtagatgaggg

SEQ ID NO: 217 - DNA, 20 bp, Artificial Sequence
  Synthetic
gtgcacacagtagatgaggg

SEQ ID NO: 218 - DNA, 20 bp, Artificial Sequence
  Synthetic
gtgcacacagtagatgaggg

SEQ ID NO: 219 - DNA, 20 bp, Artificial Sequence
  Synthetic
agggcgcagacttccaaagg

SEQ ID NO: 220 - DNA, 20 bp, Artificial Sequence
  Synthetic
aagggcgcagacttccaaag

SEQ ID NO: 221 - DNA, 20 bp, Artificial Sequence
  Synthetic
caagggcgcagacttccaaa

SEQ ID NO: 222 - DNA, 20 bp, Artificial Sequence
  Synthetic
acaagggcgcagactuccaa

SEQ ID NO: 223 - RNA, 20 nt, Artificial Sequence
  Synthetic
cacaagggcgcagacuucca

SEQ ID NO: 224 - RNA, 20 nt, Artificial Sequence
  Synthetic
gcacaagggcgcagacuucc

SEQ ID NO: 225 - RNA, 20 nt, Artificial Sequence
  Synthetic
ggcacaagggcgcagacuuc

SEQ ID NO: 226 - RNA, 20 nt, Artificial Sequence
  Synthetic
gggcacaagggcgcagacuu

SEQ ID NO: 227 - RNA, 20 nt, Artificial Sequence
  Synthetic
agggcacaagggcgcagacu

SEQ ID NO: 228 - DNA, 20 bp, Artificial Sequence
  Synthetic
cagggcacaagggcgcagac

SEQ ID NO: 229 - DNA, 20 bp, Artificial Sequence
  Synthetic
ctcagtaacattgacaccac

SEQ ID NO: 230 - RNA, 20 nt, Artificial Sequence
  Synthetic
ggcacaagggcgcagacuuc

SEQ ID NO: 231 - RNA, 20 nt, Artificial Sequence
  Synthetic
ggcacaagggcgcagacuuc

SEQ ID NO: 232 - RNA, 20 nt, Artificial Sequence
  Synthetic
ggcacaagggcacagacuuc

SEQ ID NO: 233 - RNA, 20 nt, Artificial Sequence
  Synthetic
ggcacaagggcacagacuuc

SEQ ID NO: 234 - DNA, 20 bp, Artificial Sequence
  Synthetic
gugcacacagtagatgaggg

SEQ ID NO: 235 - DNA, 20 bp, Artificial Sequence
  Synthetic
gugcacacagtagatgaggg

SEQ ID NO: 236 - DNA, 23 bp, Artificial Sequence
  Synthetic
tacagacuuccaaaggcuccgtu

SEQ ID NO: 237 - DNA, 23 bp, Artificial Sequence
  Synthetic
tcacagacuuccaaaggcucctu

SEQ ID NO: 238 - DNA, 23 bp, Artificial Sequence
  Synthetic
tgcacagacuuccaaaggcuctu

SEQ ID NO: 239 - DNA, 23 bp, Artificial Sequence
  Synthetic
tggcacagacuuccaaaggcutu

SEQ ID NO: 240 - DNA, 23 bp, Artificial Sequence
  Synthetic
tgggcacagacuuccaaaggctu

SEQ ID NO: 241 - DNA, 23 bp, Artificial Sequence
  Synthetic
tagggcacagacuuccaaaggtu

SEQ ID NO: 242 - DNA, 23 bp, Artificial Sequence
  Synthetic
taagggcacagacuuccaaagtu

SEQ ID NO: 243 - DNA, 23 bp, Artificial Sequence
  Synthetic
tcaagggcacagacuuccaaatu

SEQ ID NO: 244 - DNA, 23 bp, Artificial Sequence
  Synthetic
tacaagggcacagacuuccaatu

SEQ ID NO: 245 - DNA, 23 bp, Artificial Sequence
  Synthetic
tcacaagggcacagacuuccatu

SEQ ID NO: 246 - DNA, 23 bp, Artificial Sequence
  Synthetic
tgcacaagggcacagacuucctu

SEQ ID NO: 247 - DNA, 23 bp, Artificial Sequence
  Synthetic
tggcacaagggcacagacuuctu

SEQ ID NO: 248 - DNA, 23 bp, Artificial Sequence
  Synthetic
tgggcacaagggcacagacuutu

SEQ ID NO: 249 - DNA, 23 bp, Artificial Sequence
  Synthetic
tagggcacaagggcacagacutu

SEQ ID NO: 250 - DNA, 23 bp, Artificial Sequence
  Synthetic
tcagggcacaagggcacagactu

SEQ ID NO: 251 - DNA, 23 bp, Artificial Sequence
  Synthetic
tgcagggcacaagggcacagatu

SEQ ID NO: 252 - DNA, 23 bp, Artificial Sequence
  Synthetic
tggcagggcacaagggcacagtu

SEQ ID NO: 253 - DNA, 23 bp, Artificial Sequence
  Synthetic
taggcagggcacaagggcacatu

SEQ ID NO: 254 - DNA, 23 bp, Artificial Sequence
  Synthetic
tgaggcagggcacaagggcactu

SEQ ID NO: 255 - DNA, 23 bp, Artificial Sequence
  Synthetic
tggaggcagggcacaagggcatu

SEQ ID NO: 256 - DNA, 23 bp, Artificial Sequence
  Synthetic
tacagacuuccaaaggcuccgtu

SEQ ID NO: 257 - DNA, 23 bp, Artificial Sequence
  Synthetic
tcacagacuuccaaaggcucctu

SEQ ID NO: 258 - DNA, 23 bp, Artificial Sequence
  Synthetic
tgcacagacuuccaaaggcuctu

SEQ ID NO: 259 - DNA, 23 bp, Artificial Sequence
  Synthetic
tggcacagacuuccaaaggcutu

SEQ ID NO: 260 - DNA, 23 bp, Artificial Sequence
  Synthetic
tgggcacagacuuccaaaggctu

SEQ ID NO: 261 - DNA, 23 bp, Artificial Sequence
  Synthetic
tagggcacagacuuccaaaggtu

SEQ ID NO: 262 - DNA, 23 bp, Artificial Sequence
  Synthetic
taagggcacagacuuccaaagtu

SEQ ID NO: 263 - DNA, 23 bp, Artificial Sequence
  Synthetic
tcaagggcacagacuuccaaatu

SEQ ID NO: 264 - DNA, 23 bp, Artificial Sequence
  Synthetic
tacaagggcacagacuuccaatu

SEQ ID NO: 265 - DNA, 23 bp, Artificial Sequence
  Synthetic
tcacaagggcacagacuuccatu

SEQ ID NO: 266 - DNA, 23 bp, Artificial Sequence
  Synthetic
tgcacaagggcacagacuucctu

SEQ ID NO: 267 - DNA, 23 bp, Artificial Sequence
  Synthetic
tggcacaagggcacagacuuctu

SEQ ID NO: 268 - DNA, 23 bp, Artificial Sequence
  Synthetic
tgggcacaagggcacagacuutu

SEQ ID NO: 269 - DNA, 23 bp, Artificial Sequence
  Synthetic
tagggcacaagggcacagacutu

SEQ ID NO: 270 - DNA, 23 bp, Artificial Sequence
  Synthetic
tcagggcacaagggcacagactu

SEQ ID NO: 271 - DNA, 23 bp, Artificial Sequence
  Synthetic
tgcagggcacaagggcacagatu

SEQ ID NO: 272 - DNA, 23 bp, Artificial Sequence
  Synthetic
tggcagggcacaagggcacagtu

SEQ ID NO: 273 - DNA, 23 bp, Artificial Sequence
  Synthetic
taggcagggcacaagggcacatu

SEQ ID NO: 274 - DNA, 23 bp, Artificial Sequence
  Synthetic
tgaggcagggcacaagggcactu

SEQ ID NO: 275 - DNA, 23 bp, Artificial Sequence
  Synthetic
tggaggcagggcacaagggcatu

SEQ ID NO: 276 - RNA, 36 nt, Artificial Sequence
  Synthetic
aacaagaugaagagcaccaaguuuuagagcuaugcu

SEQ ID NO: 277 - RNA, 20 nt, Artificial Sequence
  Synthetic
ggcacaagggcacagacuuc

SEQ ID NO: 278 - RNA, 20 nt, Artificial Sequence
  Synthetic
ggcacaagggcacagacuuc

SEQ ID NO: 279 - RNA, 20 nt, Artificial Sequence
  Synthetic
ggcacaagggcacagacuuc

SEQ ID NO: 280 - RNA, 20 nt, Artificial Sequence
  Synthetic
ggcacaagggcacagacuuc

SEQ ID NO: 281 - DNA, 20 bp, Artificial Sequence
  Synthetic
gugcacacagtagatgaggg

SEQ ID NO: 282 - DNA, 20 bp, Artificial Sequence
  Synthetic
gugcacacagtagatgaggg

SEQ ID NO: 283 - DNA, 20 bp, Artificial Sequence
  Synthetic
gugcacacagtagatgaggg

SEQ ID NO: 284 - RNA, 20 nt, Artificial Sequence
  Synthetic
ggcacaagggcacagacuuc

SEQ ID NO: 285 - RNA, 20 nt, Artificial Sequence
  Synthetic
ggcacaagggcacagacuuc

SEQ ID NO: 286 - DNA, 20 bp, Artificial Sequence
  Synthetic
gugcacacagtagatgaggg

SEQ ID NO: 287 - DNA, 20 bp, Artificial Sequence
  Synthetic
gugcacacagtagatgaggg

SEQ ID NO: 288 - DNA, 20 bp, Artificial Sequence
  Synthetic
gugcacacagtagatgaggg

SEQ ID NO: 289 - RNA, 31 nt, Artificial Sequence
  Synthetic
gccuuuggaagucugcgcccuugugcccugc

SEQ ID NO: 290 - RNA, 20 nt, Artificial Sequence
  Synthetic
guggugucaauguuacugag

SEQ ID NO: 291 - DNA, 20 bp, Artificial Sequence
  Synthetic
agggcgcagacttccaaagg

SEQ ID NO: 292 - DNA, 20 bp, Artificial Sequence
  Synthetic
aagggcgcagacttccaaag

SEQ ID NO: 293 - DNA, 20 bp, Artificial Sequence
  Synthetic
caagggcgcagacttccaaa

SEQ ID NO: 294 - DNA, 20 bp, Artificial Sequence
  Synthetic
acaagggcgcagactuccaa

SEQ ID NO: 295 - RNA, 20 nt, Artificial Sequence
  Synthetic
cacaagggcgcagacuucca

SEQ ID NO: 296 - RNA, 20 nt, Artificial Sequence
  Synthetic
gcacaagggcgcagacuucc

SEQ ID NO: 297 - RNA, 20 nt, Artificial Sequence
  Synthetic
ggcacaagggcgcagacuuc

SEQ ID NO: 298 - RNA, 20 nt, Artificial Sequence
  Synthetic
gggcacaagggcgcagacuu

SEQ ID NO: 299 - RNA, 20 nt, Artificial Sequence
  Synthetic
agggcacaagggcgcagacu

SEQ ID NO: 300 - DNA, 20 bp, Artificial Sequence
  Synthetic
cagggcacaagggcgcagac

SEQ ID NO: 301 - DNA, 20 bp, Artificial Sequence
  Synthetic
cccgccgtagcctgggaccc

SEQ ID NO: 302 - DNA, 20 bp, Artificial Sequence
  Synthetic
ccccgccgtagcctgggacc

SEQ ID NO: 303 - DNA, 20 bp, Artificial Sequence
  Synthetic
catccccgccgtagccuggg

SEQ ID NO: 304 - DNA, 20 bp, Artificial Sequence
  Synthetic
ccgccatccccgccguagcc

SEQ ID NO: 305 - DNA, 20 bp, Artificial Sequence
  Synthetic
accgccatccccgccguagc

SEQ ID NO: 306 - DNA, 20 bp, Artificial Sequence
  Synthetic
uaccgccatccccgccguag

SEQ ID NO: 307 - DNA, 20 bp, Artificial Sequence
  Synthetic
utaccgccatccccgccgua

SEQ ID NO: 308 - DNA, 20 bp, Artificial Sequence
  Synthetic
gttaccgccatccccgccgu

SEQ ID NO: 309 - DNA, 20 bp, Artificial Sequence
  Synthetic
ggttaccgccatccccgccg

SEQ ID NO: 310 - DNA, 20 bp, Artificial Sequence
  Synthetic
agggutaccgccatccccgc

SEQ ID NO: 311 - DNA, 20 bp, Artificial Sequence
  Synthetic
cagggttaccgccatccccg

SEQ ID NO: 312 - DNA, 20 bp, Artificial Sequence
  Synthetic
gcagggttaccgccaucccc

SEQ ID NO: 313 - DNA, 20 bp, Artificial Sequence
  Synthetic
ugcagggttaccgccauccc

SEQ ID NO: 314 - DNA, 20 bp, Artificial Sequence
  Synthetic
agccgggggttcgtgucgcc

SEQ ID NO: 315 - DNA, 20 bp, Artificial Sequence
  Synthetic
gagccgggggttcgtgucgc

SEQ ID NO: 316 - DNA, 20 bp, Artificial Sequence
  Synthetic
ggagccgggggttcgugucg

SEQ ID NO: 317 - DNA, 20 bp, Artificial Sequence
  Synthetic
cggagccgggggttcguguc

SEQ ID NO: 318 - DNA, 20 bp, Artificial Sequence
  Synthetic
ccctcatgggctctggguug

SEQ ID NO: 319 - DNA, 20 bp, Artificial Sequence
  Synthetic
ucccucatgggctctggguu

SEQ ID NO: 320 - DNA, 20 bp, Artificial Sequence
  Synthetic
caggagggggcgggtguccc

SEQ ID NO: 321 - RNA, 20 nt, Artificial Sequence
  Synthetic
ccaggagggggcgggugucc

SEQ ID NO: 322 - DNA, 20 bp, Artificial Sequence
  Synthetic
cccaagtgagggagcggggc

SEQ ID NO: 323 - RNA, 20 nt, Artificial Sequence
  Synthetic
aggccccaacaaggcucugc

SEQ ID NO: 324 - DNA, 20 bp, Artificial Sequence
  Synthetic
ggctctgggttgctggguca

SEQ ID NO: 325 - DNA, 20 bp, Artificial Sequence
  Synthetic
gtccctcatgggctcugggu

SEQ ID NO: 326 - DNA, 20 bp, Artificial Sequence
  Synthetic
gtgtccctcatgggcucugg

SEQ ID NO: 327 - DNA, 20 bp, Artificial Sequence
  Synthetic
ccatccccgccgtagccugg

SEQ ID NO: 328 - DNA, 20 bp, Artificial Sequence
  Synthetic
gccauccccgccgtagccug

SEQ ID NO: 329 - DNA, 20 bp, Artificial Sequence
  Synthetic
cgccatccccgccgtagccu

SEQ ID NO: 330 - DNA, 20 bp, Artificial Sequence
  Synthetic
gggtuaccgccatccccgcc

SEQ ID NO: 331 - DNA, 20 bp, Artificial Sequence
  Synthetic
gggcuctgggttgctggguc

SEQ ID NO: 332 - DNA, 20 bp, Artificial Sequence
  Synthetic
aggagggggcgggtgucccu

SEQ ID NO: 333 - DNA, 20 bp, Artificial Sequence
  Synthetic
ggtguccctcatgggcucug

SEQ ID NO: 334 - DNA, 20 bp, Artificial Sequence
  Synthetic
ugtccctcatgggctcuggg

SEQ ID NO: 335 - DNA, 20 bp, Artificial Sequence
  Synthetic
ugggctctgggttgcugggu

SEQ ID NO: 336 - DNA, 20 bp, Artificial Sequence
  Synthetic
acagugttggccatgcccag

SEQ ID NO: 337 - DNA, 20 bp, Artificial Sequence
  Synthetic
accgcgaccctctggacagg

SEQ ID NO: 338 - DNA, 20 bp, Artificial Sequence
  Synthetic
uaccgcgaccctctggacag

SEQ ID NO: 339 - DNA, 20 bp, Artificial Sequence
  Synthetic
gtaccgcgaccctctggaca

SEQ ID NO: 340 - DNA, 20 bp, Artificial Sequence
  Synthetic
ggtaccgcgaccctcuggac

SEQ ID NO: 341 - DNA, 20 bp, Artificial Sequence
  Synthetic
aggtaccgcgaccctcugga

SEQ ID NO: 342 - DNA, 20 bp, Artificial Sequence
  Synthetic
cagggaggtaccgcgacccu

SEQ ID NO: 343 - DNA, 20 bp, Artificial Sequence
  Synthetic
ucagggaggtaccgcgaccc

SEQ ID NO: 344 - DNA, 20 bp, Artificial Sequence
  Synthetic
ctcagggaggtaccgcgacc

SEQ ID NO: 345 - DNA, 20 bp, Artificial Sequence
  Synthetic
cctcagggaggtaccgcgac

SEQ ID NO: 346 - DNA, 20 bp, Artificial Sequence
  Synthetic
ggaaagcctggcctcaggga

SEQ ID NO: 347 - DNA, 20 bp, Artificial Sequence
  Synthetic
atgcccagcacgcaggccag

SEQ ID NO: 348 - RNA, 20 nt, Artificial Sequence
  Synthetic
gccaugcccagcacgcaggc

SEQ ID NO: 349 - DNA, 20 bp, Artificial Sequence
  Synthetic
cagcacgcaggccaggggcg

SEQ ID NO: 350 - DNA, 20 bp, Artificial Sequence
  Synthetic
catgcccagcacgcaggcca

SEQ ID NO: 351 - DNA, 20 bp, Artificial Sequence
  Synthetic
ccatgcccagcacgcaggcc

SEQ ID NO: 352 - DNA, 20 bp, Artificial Sequence
  Synthetic
gcgaccctctggacagggaa

SEQ ID NO: 353 - RNA, 20 nt, Artificial Sequence
  Synthetic
ugcccagcacgcaggccagg

SEQ ID NO: 354 - DNA, 20 bp, Artificial Sequence
  Synthetic
aaagcctggcctcagggagg

SEQ ID NO: 355 - DNA, 20 bp, Artificial Sequence
  Synthetic
gcccagcacgcaggccaggg

SEQ ID NO: 356 - DNA, 20 bp, Artificial Sequence
  Synthetic
cccagcacgcaggccagggg

SEQ ID NO: 357 - DNA, 20 bp, Artificial Sequence
  Synthetic
ggccatgcccagcacgcagg

SEQ ID NO: 358 - DNA, 20 bp, Artificial Sequence
  Synthetic
cgcaggccaggggcgcgggg

SEQ ID NO: 359 - DNA, 20 bp, Artificial Sequence
  Synthetic
agcacgcaggccaggggcgc

SEQ ID NO: 360 - DNA, 20 bp, Artificial Sequence
  Synthetic
ccagcacgcaggccaggggc

SEQ ID NO: 361 - DNA, 20 bp, Artificial Sequence
  Synthetic
gcaggccaggggcgcggggc

SEQ ID NO: 362 - RNA, 16 nt, Artificial Sequence
  Synthetic
cacaagggcgcagacu

SEQ ID NO: 363 - RNA, 12 nt, Artificial Sequence
  Synthetic
agggcgcagacu

SEQ ID NO: 364 - DNA, 20 bp, Artificial Sequence
  Synthetic
gugcacacagtggatgaggg

SEQ ID NO: 365 - DNA, 16 bp, Artificial Sequence
  Synthetic
gcacacagtggatgag

SEQ ID NO: 366 - DNA, 12 bp, Artificial Sequence
  Synthetic
acagtggatgag

SEQ ID NO: 367 - DNA, 12 bp, Artificial Sequence
  Synthetic
acagtggatgag

SEQ ID NO: 368 - DNA, 20 bp, Artificial Sequence
  Synthetic
gagcgtagaaaccctccaaa

SEQ ID NO: 369 - DNA, 20 bp, Artificial Sequence
  Synthetic
ugagcgtagaaacccuccaa

SEQ ID NO: 370 - DNA, 20 bp, Artificial Sequence
  Synthetic
cugagcgtagaaacccucca

SEQ ID NO: 371 - DNA, 20 bp, Artificial Sequence
  Synthetic
gcugagcgtagaaacccucc

SEQ ID NO: 372 - DNA, 20 bp, Artificial Sequence
  Synthetic
ggcugagcgtagaaacccuc

SEQ ID NO: 373 - DNA, 20 bp, Artificial Sequence
  Synthetic
aggcugagcgtagaaacccu

SEQ ID NO: 374 - DNA, 20 bp, Artificial Sequence
  Synthetic
aaggctgagcgtagaaaccc

SEQ ID NO: 375 - DNA, 20 bp, Artificial Sequence
  Synthetic
caaggctgagcgtagaaacc

SEQ ID NO: 376 - DNA, 20 bp, Artificial Sequence
  Synthetic
ccaaggctgagcgtagaaac

SEQ ID NO: 377 - DNA, 20 bp, Artificial Sequence
  Synthetic
uccaaggctgagcgtagaaa

SEQ ID NO: 378 - DNA, 20 bp, Artificial Sequence
  Synthetic
gagcgtagaaaccctccaaa

SEQ ID NO: 379 - DNA, 20 bp, Artificial Sequence
  Synthetic
ugagcgtagaaacccuccaa

SEQ ID NO: 380 - DNA, 20 bp, Artificial Sequence
  Synthetic
cugagcgtagaaacccucca

SEQ ID NO: 381 - DNA, 20 bp, Artificial Sequence
  Synthetic
gcugagcgtagaaacccucc

SEQ ID NO: 382 - DNA, 20 bp, Artificial Sequence
  Synthetic
ggcugagcgtagaaacccuc

SEQ ID NO: 383 - DNA, 20 bp, Artificial Sequence
  Synthetic
aggcugagcgtagaaacccu

SEQ ID NO: 384 - DNA, 20 bp, Artificial Sequence
  Synthetic
aaggctgagcgtagaaaccc

SEQ ID NO: 385 - DNA, 20 bp, Artificial Sequence
  Synthetic
caaggctgagcgtagaaacc

SEQ ID NO: 386 - DNA, 20 bp, Artificial Sequence
  Synthetic
ccaaggctgagcgtagaaac

SEQ ID NO: 387 - DNA, 20 bp, Artificial Sequence
  Synthetic
uccaaggctgagcgtagaaa

SEQ ID NO: 388 - DNA, 20 bp, Artificial Sequence
  Synthetic
gagcgtagaaaccctccaaa

SEQ ID NO: 389 - DNA, 20 bp, Artificial Sequence
  Synthetic
tgagcgtagaaaccctccaa

SEQ ID NO: 390 - DNA, 20 bp, Artificial Sequence
  Synthetic
ctgagcgtagaaaccctcca

SEQ ID NO: 391 - DNA, 20 bp, Artificial Sequence
  Synthetic
gctgagcgtagaaaccctcc

SEQ ID NO: 392 - DNA, 20 bp, Artificial Sequence
  Synthetic
ggctgagcgtagaaaccctc

SEQ ID NO: 393 - DNA, 20 bp, Artificial Sequence
  Synthetic
aggctgagcgtagaaaccct

SEQ ID NO: 394 - DNA, 20 bp, Artificial Sequence
  Synthetic
aaggctgagcgtagaaaccc

SEQ ID NO: 395 - DNA, 20 bp, Artificial Sequence
  Synthetic
caaggctgagcgtagaaacc

SEQ ID NO: 396 - DNA, 20 bp, Artificial Sequence
  Synthetic
ccaaggctgagcgtagaaac

SEQ ID NO: 397 - DNA, 20 bp, Artificial Sequence
  Synthetic
tccaaggctgagcgtagaaa

SEQ ID NO: 398 - DNA, 20 bp, Artificial Sequence
  Synthetic
gagcgtagaaaccctccaaa

SEQ ID NO: 399 - DNA, 20 bp, Artificial Sequence
  Synthetic
tgagcgtagaaaccctccaa

SEQ ID NO: 400 - DNA, 20 bp, Artificial Sequence
  Synthetic
ctgagcgtagaaaccctcca

SEQ ID NO: 401 - DNA, 20 bp, Artificial Sequence
  Synthetic
gctgagcgtagaaaccctcc

SEQ ID NO: 402 - DNA, 20 bp, Artificial Sequence
  Synthetic
ggctgagcgtagaaaccctc

SEQ ID NO: 403 - DNA, 20 bp, Artificial Sequence
  Synthetic
aggctgagcgtagaaaccct

SEQ ID NO: 404 - DNA, 20 bp, Artificial Sequence
  Synthetic
aaggctgagcgtagaaaccc

SEQ ID NO: 405 - DNA, 20 bp, Artificial Sequence
  Synthetic
caaggctgagcgtagaaacc

SEQ ID NO: 406 - DNA, 20 bp, Artificial Sequence
  Synthetic
ccaaggctgagcgtagaaac

SEQ ID NO: 407 - DNA, 20 bp, Artificial Sequence
  Synthetic
tccaaggctgagcgtagaaa

SEQ ID NO: 408 - DNA, 20 bp, Artificial Sequence
  Synthetic
gagcgtagaaaccctccaaa

SEQ ID NO: 409 - DNA, 20 bp, Artificial Sequence
  Synthetic
ugagcgtagaaacccuccaa

SEQ ID NO: 410 - DNA, 20 bp, Artificial Sequence
  Synthetic
ctgagcgtagaaacccucca

SEQ ID NO: 411 - DNA, 20 bp, Artificial Sequence
  Synthetic
gctgagcgtagaaacccucc

SEQ ID NO: 412 - DNA, 20 bp, Artificial Sequence
  Synthetic
ggctgagcgtagaaacccuc

SEQ ID NO: 413 - DNA, 20 bp, Artificial Sequence
  Synthetic
aggcugagcgtagaaacccu

SEQ ID NO: 414 - DNA, 20 bp, Artificial Sequence
  Synthetic
aaggctgagcgtagaaaccc

SEQ ID NO: 415 - DNA, 20 bp, Artificial Sequence
  Synthetic
caaggctgagcgtagaaacc

SEQ ID NO: 416 - DNA, 20 bp, Artificial Sequence
  Synthetic
ccaaggctgagcgtagaaac

SEQ ID NO: 417 - DNA, 20 bp, Artificial Sequence
  Synthetic
uccaaggctgagcgtagaaa

SEQ ID NO: 418 - RNA, 20 nt, Artificial Sequence
  Synthetic
aucugaagcagcagcuucuc

SEQ ID NO: 419 - RNA, 20 nt, Artificial Sequence
  Synthetic
gaucugaagcagcagcuucu

SEQ ID NO: 420 - DNA, 20 bp, Artificial Sequence
  Synthetic
ugauctgaagcagcagcuuc

SEQ ID NO: 421 - DNA, 20 bp, Artificial Sequence
  Synthetic
uugauctgaagcagcagcuu

SEQ ID NO: 422 - DNA, 20 bp, Artificial Sequence
  Synthetic
guugatctgaagcagcagcu

SEQ ID NO: 423 - DNA, 20 bp, Artificial Sequence
  Synthetic
gguugatctgaagcagcagc

SEQ ID NO: 424 - DNA, 20 bp, Artificial Sequence
  Synthetic
ggguugatctgaagcagcag

SEQ ID NO: 425 - DNA, 20 bp, Artificial Sequence
  Synthetic
ggggutgatctgaagcagca

SEQ ID NO: 426 - DNA, 20 bp, Artificial Sequence
  Synthetic
cggggttgatctgaagcagc

SEQ ID NO: 427 - DNA, 20 bp, Artificial Sequence
  Synthetic
ucggggttgatctgaagcag

SEQ ID NO: 428 - RNA, 20 nt, Artificial Sequence
  Synthetic
aucugaagcagcagcuucuc

SEQ ID NO: 429 - RNA, 20 nt, Artificial Sequence
  Synthetic
gaucugaagcagcagcuucu

SEQ ID NO: 430 - DNA, 20 bp, Artificial Sequence
  Synthetic
ugauctgaagcagcagcuuc

SEQ ID NO: 431 - DNA, 20 bp, Artificial Sequence
  Synthetic
uugauctgaagcagcagcuu

SEQ ID NO: 432 - DNA, 20 bp, Artificial Sequence
  Synthetic
guugatctgaagcagcagcu

SEQ ID NO: 433 - DNA, 20 bp, Artificial Sequence
  Synthetic
gguugatctgaagcagcagc

SEQ ID NO: 434 - DNA, 20 bp, Artificial Sequence
  Synthetic
ggguugatctgaagcagcag

SEQ ID NO: 435 - DNA, 20 bp, Artificial Sequence
  Synthetic
ggggutgatctgaagcagca

SEQ ID NO: 436 - DNA, 20 bp, Artificial Sequence
  Synthetic
cggggttgatctgaagcagc

SEQ ID NO: 437 - DNA, 20 bp, Artificial Sequence
  Synthetic
ucggggttgatctgaagcag

SEQ ID NO: 438 - DNA, 20 bp, Artificial Sequence
  Synthetic
atctgaagcagcagcttctc

SEQ ID NO: 439 - DNA, 20 bp, Artificial Sequence
  Synthetic
gatctgaagcagcagcttct

SEQ ID NO: 440 - DNA, 20 bp, Artificial Sequence
  Synthetic
tgatctgaagcagcagcttc

SEQ ID NO: 441 - DNA, 20 bp, Artificial Sequence
  Synthetic
ttgatctgaagcagcagctt

SEQ ID NO: 442 - DNA, 20 bp, Artificial Sequence
  Synthetic
gttgatctgaagcagcagct

SEQ ID NO: 443 - DNA, 20 bp, Artificial Sequence
  Synthetic
ggttgatctgaagcagcagc

SEQ ID NO: 444 - DNA, 20 bp, Artificial Sequence
  Synthetic
gggttgatctgaagcagcag

SEQ ID NO: 445 - DNA, 20 bp, Artificial Sequence
  Synthetic
ggggttgatctgaagcagca

SEQ ID NO: 446 - DNA, 20 bp, Artificial Sequence
  Synthetic
cggggttgatctgaagcagc

SEQ ID NO: 447 - DNA, 20 bp, Artificial Sequence
  Synthetic
tcggggttgatctgaagcag

SEQ ID NO: 448 - DNA, 20 bp, Artificial Sequence
  Synthetic
atctgaagcagcagcttctc

SEQ ID NO: 449 - DNA, 20 bp, Artificial Sequence
  Synthetic
gatctgaagcagcagcttct

SEQ ID NO: 450 - DNA, 20 bp, Artificial Sequence
  Synthetic
tgatctgaagcagcagcttc

SEQ ID NO: 451 - DNA, 20 bp, Artificial Sequence
  Synthetic
ttgatctgaagcagcagctt

SEQ ID NO: 452 - DNA, 20 bp, Artificial Sequence
  Synthetic
gttgatctgaagcagcagct

SEQ ID NO: 453 - DNA, 20 bp, Artificial Sequence
  Synthetic
ggttgatctgaagcagcagc

SEQ ID NO: 454 - DNA, 20 bp, Artificial Sequence
  Synthetic
gggttgatctgaagcagcag

SEQ ID NO: 455 - DNA, 20 bp, Artificial Sequence
  Synthetic
ggggttgatctgaagcagca

SEQ ID NO: 456 - DNA, 20 bp, Artificial Sequence
  Synthetic
cggggttgatctgaagcagc

SEQ ID NO: 457 - DNA, 20 bp, Artificial Sequence
  Synthetic
tcggggttgatctgaagcag

SEQ ID NO: 458 - DNA, 20 bp, Artificial Sequence
  Synthetic
atctgaagcagcagcuucuc

SEQ ID NO: 459 - DNA, 20 bp, Artificial Sequence
  Synthetic
gatcugaagcagcagcuucu

SEQ ID NO: 460 - DNA, 20 bp, Artificial Sequence
  Synthetic
ugatctgaagcagcagcuuc

SEQ ID NO: 461 - DNA, 20 bp, Artificial Sequence
  Synthetic
utgauctgaagcagcagcuu

SEQ ID NO: 462 - DNA, 20 bp, Artificial Sequence
  Synthetic
gttgatctgaagcagcagcu

SEQ ID NO: 463 - DNA, 20 bp, Artificial Sequence
  Synthetic
ggttgatctgaagcagcagc

SEQ ID NO: 464 - DNA, 20 bp, Artificial Sequence
  Synthetic
gggtugatctgaagcagcag

SEQ ID NO: 465 - DNA, 20 bp, Artificial Sequence
  Synthetic
ggggutgatctgaagcagca

SEQ ID NO: 466 - DNA, 20 bp, Artificial Sequence
  Synthetic
cggggttgatctgaagcagc

SEQ ID NO: 467 - DNA, 20 bp, Artificial Sequence
  Synthetic
ucggggttgatctgaagcag

SEQ ID NO: 468 - DNA, 20 bp, Artificial Sequence
  Synthetic
gaggaggccgtgcagggcuc

SEQ ID NO: 469 - DNA, 20 bp, Artificial Sequence
  Synthetic
agaggaggccgtgcagggcu

SEQ ID NO: 470 - DNA, 20 bp, Artificial Sequence
  Synthetic
uagaggaggccgtgcagggc

SEQ ID NO: 471 - DNA, 20 bp, Artificial Sequence
  Synthetic
auagaggaggccgtgcaggg

SEQ ID NO: 472 - DNA, 20 bp, Artificial Sequence
  Synthetic
cauagaggaggccgtgcagg

SEQ ID NO: 473 - RNA, 20 nt, Artificial Sequence
  Synthetic
acauagaggaggccgugcag

SEQ ID NO: 474 - RNA, 20 nt, Artificial Sequence
  Synthetic
cacauagaggaggccgugca

SEQ ID NO: 475 - DNA, 20 bp, Artificial Sequence
  Synthetic
gcacatagaggaggccgugc

SEQ ID NO: 476 - DNA, 20 bp, Artificial Sequence
  Synthetic
agcacatagaggaggccgug

SEQ ID NO: 477 - DNA, 20 bp, Artificial Sequence
  Synthetic
cagcacatagaggaggccgu

SEQ ID NO: 478 - DNA, 20 bp, Artificial Sequence
  Synthetic
gaggaggccgtgcagggcuc

SEQ ID NO: 479 - DNA, 20 bp, Artificial Sequence
  Synthetic
agaggaggccgtgcagggcu

SEQ ID NO: 480 - DNA, 20 bp, Artificial Sequence
  Synthetic
uagaggaggccgtgcagggc

SEQ ID NO: 481 - DNA, 20 bp, Artificial Sequence
  Synthetic
auagaggaggccgtgcaggg

SEQ ID NO: 482 - DNA, 20 bp, Artificial Sequence
  Synthetic
cauagaggaggccgtgcagg

SEQ ID NO: 483 - RNA, 20 nt, Artificial Sequence
  Synthetic
acauagaggaggccgugcag

SEQ ID NO: 484 - RNA, 20 nt, Artificial Sequence
  Synthetic
cacauagaggaggccgugca

SEQ ID NO: 485 - DNA, 20 bp, Artificial Sequence
  Synthetic
gcacatagaggaggccgugc

SEQ ID NO: 486 - DNA, 20 bp, Artificial Sequence
  Synthetic
agcacatagaggaggccgug

SEQ ID NO: 487 - DNA, 20 bp, Artificial Sequence
  Synthetic
cagcacatagaggaggccgu

SEQ ID NO: 488 - DNA, 20 bp, Artificial Sequence
  Synthetic
gaggaggccgtgcagggctc

SEQ ID NO: 489 - DNA, 20 bp, Artificial Sequence
  Synthetic
agaggaggccgtgcagggct

SEQ ID NO: 490 - DNA, 20 bp, Artificial Sequence
  Synthetic
tagaggaggccgtgcagggc

SEQ ID NO: 491 - DNA, 20 bp, Artificial Sequence
  Synthetic
atagaggaggccgtgcaggg

SEQ ID NO: 492 - DNA, 20 bp, Artificial Sequence
  Synthetic
catagaggaggccgtgcagg

SEQ ID NO: 493 - DNA, 20 bp, Artificial Sequence
  Synthetic
acatagaggaggccgtgcag

SEQ ID NO: 494 - DNA, 20 bp, Artificial Sequence
  Synthetic
cacatagaggaggccgtgca

SEQ ID NO: 495 - DNA, 20 bp, Artificial Sequence
  Synthetic
gcacatagaggaggccgtgc

SEQ ID NO: 496 - DNA, 20 bp, Artificial Sequence
  Synthetic
agcacatagaggaggccgtg

SEQ ID NO: 497 - DNA, 20 bp, Artificial Sequence
  Synthetic
cagcacatagaggaggccgt

SEQ ID NO: 498 - DNA, 20 bp, Artificial Sequence
  Synthetic
gaggaggccgtgcagggctc

SEQ ID NO: 499 - DNA, 20 bp, Artificial Sequence
  Synthetic
agaggaggccgtgcagggct

SEQ ID NO: 500 - DNA, 20 bp, Artificial Sequence
  Synthetic
tagaggaggccgtgcagggc

SEQ ID NO: 501 - DNA, 20 bp, Artificial Sequence
  Synthetic
atagaggaggccgtgcaggg

SEQ ID NO: 502 - DNA, 20 bp, Artificial Sequence
  Synthetic
catagaggaggccgtgcagg

SEQ ID NO: 503 - DNA, 20 bp, Artificial Sequence
  Synthetic
acatagaggaggccgtgcag

SEQ ID NO: 504 - DNA, 20 bp, Artificial Sequence
  Synthetic
cacatagaggaggccgtgca

SEQ ID NO: 505 - DNA, 20 bp, Artificial Sequence
  Synthetic
gcacatagaggaggccgtgc

SEQ ID NO: 506 - DNA, 20 bp, Artificial Sequence
  Synthetic
agcacatagaggaggccgtg

SEQ ID NO: 507 - DNA, 20 bp, Artificial Sequence
  Synthetic
cagcacatagaggaggccgt

SEQ ID NO: 508 - DNA, 20 bp, Artificial Sequence
  Synthetic
gaggaggccgtgcagggcuc

SEQ ID NO: 509 - DNA, 20 bp, Artificial Sequence
  Synthetic
agaggaggccgtgcagggcu

SEQ ID NO: 510 - DNA, 20 bp, Artificial Sequence
  Synthetic
uagaggaggccgtgcagggc

SEQ ID NO: 511 - DNA, 20 bp, Artificial Sequence
  Synthetic
atagaggaggccgtgcaggg

SEQ ID NO: 512 - DNA, 20 bp, Artificial Sequence
  Synthetic
catagaggaggccgtgcagg

SEQ ID NO: 513 - DNA, 20 bp, Artificial Sequence
  Synthetic
acatagaggaggccgugcag

SEQ ID NO: 514 - RNA, 20 nt, Artificial Sequence
  Synthetic
cacauagaggaggccgugca

SEQ ID NO: 515 - DNA, 20 bp, Artificial Sequence
  Synthetic
gcacatagaggaggccgugc

SEQ ID NO: 516 - DNA, 20 bp, Artificial Sequence
  Synthetic
agcacatagaggaggccgug

SEQ ID NO: 517 - DNA, 20 bp, Artificial Sequence
  Synthetic
cagcacatagaggaggccgu

SEQ ID NO: 518 - DNA, 20 bp, Artificial Sequence
  Synthetic
acaguagatgagggaggagg

SEQ ID NO: 519 - DNA, 20 bp, Artificial Sequence
  Synthetic
cacagtagatgagggaggag

SEQ ID NO: 520 - DNA, 20 bp, Artificial Sequence
  Synthetic
acacagtagatgagggagga

SEQ ID NO: 521 - DNA, 20 bp, Artificial Sequence
  Synthetic
cacacagtagatgagggagg

SEQ ID NO: 522 - DNA, 20 bp, Artificial Sequence
  Synthetic
gcacacagtagatgagggag

SEQ ID NO: 523 - DNA, 20 bp, Artificial Sequence
  Synthetic
ugcacacagtagatgaggga

SEQ ID NO: 524 - DNA, 20 bp, Artificial Sequence
  Synthetic
agtgcacacagtagaugagg

SEQ ID NO: 525 - DNA, 20 bp, Artificial Sequence
  Synthetic
aagtgcacacagtagaugag

SEQ ID NO: 526 - DNA, 20 bp, Artificial Sequence
  Synthetic
gaagugcacacagtagauga

SEQ ID NO: 527 - DNA, 20 bp, Artificial Sequence
  Synthetic
acaguagatgagggaggagg

SEQ ID NO: 528 - DNA, 20 bp, Artificial Sequence
  Synthetic
cacagtagatgagggaggag

SEQ ID NO: 529 - DNA, 20 bp, Artificial Sequence
  Synthetic
acacagtagatgagggagga

SEQ ID NO: 530 - DNA, 20 bp, Artificial Sequence
  Synthetic
cacacagtagatgagggagg

SEQ ID NO: 531 - DNA, 20 bp, Artificial Sequence
  Synthetic
gcacacagtagatgagggag

SEQ ID NO: 532 - DNA, 20 bp, Artificial Sequence
  Synthetic
ugcacacagtagatgaggga

SEQ ID NO: 533 - DNA, 20 bp, Artificial Sequence
  Synthetic
agtgcacacagtagaugagg

SEQ ID NO: 534 - DNA, 20 bp, Artificial Sequence
  Synthetic
aagtgcacacagtagaugag

SEQ ID NO: 535 - DNA, 20 bp, Artificial Sequence
  Synthetic
gaagugcacacagtagauga

SEQ ID NO: 536 - DNA, 23 bp, Artificial Sequence
  Synthetic
tuaggaggcagaagcagacgutu

SEQ ID NO: 537 - DNA, 20 bp, Artificial Sequence
  Synthetic
agggcgcagacttccaaagg

SEQ ID NO: 538 - DNA, 20 bp, Artificial Sequence
  Synthetic
aagggcgcagacttccaaag

SEQ ID NO: 539 - DNA, 20 bp, Artificial Sequence
  Synthetic
caagggcgcagacttccaaa

SEQ ID NO: 540 - DNA, 20 bp, Artificial Sequence
  Synthetic
acaagggcgcagacttccaa

SEQ ID NO: 541 - DNA, 20 bp, Artificial Sequence
  Synthetic
cacaagggcgcagacttcca

SEQ ID NO: 542 - DNA, 20 bp, Artificial Sequence
  Synthetic
gcacaagggcgcagacttcc

SEQ ID NO: 543 - DNA, 20 bp, Artificial Sequence
  Synthetic
ggcacaagggcgcagacttc

SEQ ID NO: 544 - DNA, 20 bp, Artificial Sequence
  Synthetic
gggcacaagggcgcagactt

SEQ ID NO: 545 - DNA, 20 bp, Artificial Sequence
  Synthetic
agggcacaagggcgcagact

SEQ ID NO: 546 - DNA, 20 bp, Artificial Sequence
  Synthetic
cagggcacaagggcgcagac

SEQ ID NO: 547 - DNA, 20 bp, Artificial Sequence
  Synthetic
acaguggatgagggaggagg

SEQ ID NO: 548 - DNA, 20 bp, Artificial Sequence
  Synthetic
cacagtggatgagggaggag

SEQ ID NO: 549 - DNA, 20 bp, Artificial Sequence
  Synthetic
acacagtggatgagggagga

SEQ ID NO: 550 - DNA, 20 bp, Artificial Sequence
  Synthetic
cacacagtggatgagggagg

SEQ ID NO: 551 - DNA, 20 bp, Artificial Sequence
  Synthetic
gcacacagtggatgagggag

SEQ ID NO: 552 - DNA, 20 bp, Artificial Sequence
  Synthetic
ugcacacagtggatgaggga

SEQ ID NO: 553 - DNA, 20 bp, Artificial Sequence
  Synthetic
gtgcacacagtggatgaggg

SEQ ID NO: 554 - DNA, 20 bp, Artificial Sequence
  Synthetic
agtgcacacagtggaugagg

SEQ ID NO: 555 - DNA, 20 bp, Artificial Sequence
  Synthetic
aagtgcacacagtggaugag

SEQ ID NO: 556 - DNA, 20 bp, Artificial Sequence
  Synthetic
gaagugcacacagtggauga

SEQ ID NO: 557 - DNA, 20 bp, Artificial Sequence
  Synthetic
acaguggatgagggaggagg

SEQ ID NO: 558 - DNA, 20 bp, Artificial Sequence
  Synthetic
cacagtggatgagggaggag

SEQ ID NO: 559 - DNA, 20 bp, Artificial Sequence
  Synthetic
acacagtggatgagggagga

SEQ ID NO: 560 - DNA, 20 bp, Artificial Sequence
  Synthetic
cacacagtggatgagggagg

SEQ ID NO: 561 - DNA, 20 bp, Artificial Sequence
  Synthetic
gcacacagtggatgagggag

SEQ ID NO: 562 - DNA, 20 bp, Artificial Sequence
  Synthetic
ugcacacagtggatgaggga

SEQ ID NO: 563 - DNA, 20 bp, Artificial Sequence
  Synthetic
gtgcacacagtggatgaggg

SEQ ID NO: 564 - DNA, 20 bp, Artificial Sequence
  Synthetic
agtgcacacagtggaugagg

SEQ ID NO: 565 - DNA, 20 bp, Artificial Sequence
  Synthetic
aagtgcacacagtggaugag

SEQ ID NO: 566 - DNA, 20 bp, Artificial Sequence
  Synthetic
gaagugcacacagtggauga

SEQ ID NO: 567 - DNA, 18 bp, Artificial Sequence
  Synthetic
uucuctattgcacauucc

SEQ ID NO: 568 - DNA, 18 bp, Artificial Sequence
  Synthetic
uucuctattgcacauucc

SEQ ID NO: 569 - DNA, 18 bp, Artificial Sequence
  Synthetic
uucuctattgcacauucc

SEQ ID NO: 570 - DNA, 18 bp, Artificial Sequence
  Synthetic
uucuctattgcacauucc

SEQ ID NO: 571 - DNA, 20 bp, Artificial Sequence
  Synthetic
agggcgcagacttccaaagg

SEQ ID NO: 572 - DNA, 20 bp, Artificial Sequence
  Synthetic
aagggcgcagacttccaaag

SEQ ID NO: 573 - DNA, 20 bp, Artificial Sequence
  Synthetic
caagggcgcagacttccaaa

SEQ ID NO: 574 - DNA, 20 bp, Artificial Sequence
  Synthetic
acaagggcgcagacttccaa

SEQ ID NO: 575 - DNA, 20 bp, Artificial Sequence
  Synthetic
cacaagggcgcagacttcca

SEQ ID NO: 576 - DNA, 20 bp, Artificial Sequence
  Synthetic
gcacaagggcgcagacttcc

SEQ ID NO: 577 - DNA, 20 bp, Artificial Sequence
  Synthetic
ggcacaagggcgcagacttc

SEQ ID NO: 578 - DNA, 20 bp, Artificial Sequence
  Synthetic
gggcacaagggcgcagactt

SEQ ID NO: 579 - DNA, 20 bp, Artificial Sequence
  Synthetic
agggcacaagggcgcagact

SEQ ID NO: 580 - DNA, 20 bp, Artificial Sequence
  Synthetic
cagggcacaagggcgcagac

SEQ ID NO: 581 - DNA, 20 bp, Artificial Sequence
  Synthetic
agggcgcagacttccaaagg

SEQ ID NO: 582 - DNA, 20 bp, Artificial Sequence
  Synthetic
aagggcgcagacttccaaag

SEQ ID NO: 583 - DNA, 20 bp, Artificial Sequence
  Synthetic
caagggcgcagacttccaaa

SEQ ID NO: 584 - DNA, 20 bp, Artificial Sequence
  Synthetic
acaagggcgcagacttccaa

SEQ ID NO: 585 - DNA, 20 bp, Artificial Sequence
  Synthetic
cacaagggcgcagacttcca

SEQ ID NO: 586 - DNA, 20 bp, Artificial Sequence
  Synthetic
gcacaagggcgcagacttcc

SEQ ID NO: 587 - DNA, 20 bp, Artificial Sequence
  Synthetic
ggcacaagggcgcagacttc

SEQ ID NO: 588 - DNA, 20 bp, Artificial Sequence
  Synthetic
gggcacaagggcgcagactt

SEQ ID NO: 589 - DNA, 20 bp, Artificial Sequence
  Synthetic
agggcacaagggcgcagact

SEQ ID NO: 590 - DNA, 20 bp, Artificial Sequence
  Synthetic
cagggcacaagggcgcagac

SEQ ID NO: 591 - DNA, 20 bp, Artificial Sequence
  Synthetic
agggcgcagacttccaaagg

SEQ ID NO: 592 - DNA, 20 bp, Artificial Sequence
  Synthetic
aagggcgcagacttccaaag

SEQ ID NO: 593 - DNA, 20 bp, Artificial Sequence
  Synthetic
caagggcgcagacttccaaa

SEQ ID NO: 594 - DNA, 20 bp, Artificial Sequence
  Synthetic
acaagggcgcagactuccaa

SEQ ID NO: 595 - RNA, 20 nt, Artificial Sequence
  Synthetic
cacaagggcgcagacuucca

SEQ ID NO: 596 - RNA, 20 nt, Artificial Sequence
  Synthetic
gcacaagggcgcagacuucc

SEQ ID NO: 597 - RNA, 20 nt, Artificial Sequence
  Synthetic
ggcacaagggcgcagacuuc

SEQ ID NO: 598 - DNA, 20 bp, Artificial Sequence
  Synthetic
gggcacaagggcgcagacut

SEQ ID NO: 599 - DNA, 20 bp, Artificial Sequence
  Synthetic
agggcacaagggcgcagact

SEQ ID NO: 600 - DNA, 20 bp, Artificial Sequence
  Synthetic
cagggcacaagggcgcagac

SEQ ID NO: 601 - DNA, 20 bp, Artificial Sequence
  Synthetic
agggcgcagacttccaaagg

SEQ ID NO: 602 - DNA, 20 bp, Artificial Sequence
  Synthetic
aagggcgcagacttccaaag

SEQ ID NO: 603 - DNA, 20 bp, Artificial Sequence
  Synthetic
caagggcgcagacttccaaa

SEQ ID NO: 604 - DNA, 20 bp, Artificial Sequence
  Synthetic
acaagggcgcagacttccaa

SEQ ID NO: 605 - DNA, 20 bp, Artificial Sequence
  Synthetic
cacaagggcgcagacttcca

SEQ ID NO: 606 - DNA, 20 bp, Artificial Sequence
  Synthetic
gcacaagggcgcagacttcc

SEQ ID NO: 607 - DNA, 20 bp, Artificial Sequence
  Synthetic
ggcacaagggcgcagacttc

SEQ ID NO: 608 - DNA, 20 bp, Artificial Sequence
  Synthetic
gggcacaagggcgcagactt

SEQ ID NO: 609 - DNA, 20 bp, Artificial Sequence
  Synthetic
agggcacaagggcgcagact

SEQ ID NO: 610 - DNA, 20 bp, Artificial Sequence
  Synthetic
cagggcacaagggcgcagac

SEQ ID NO: 611 - DNA, 23 bp, Artificial Sequence
  Synthetic
tgcagacuuccaaaggcuccgtu

SEQ ID NO: 612 - DNA, 23 bp, Artificial Sequence
  Synthetic
tcgcagacuuccaaaggcucctu

SEQ ID NO: 613 - DNA, 23 bp, Artificial Sequence
  Synthetic
tgcgcagacuuccaaaggcuctu

SEQ ID NO: 614 - DNA, 23 bp, Artificial Sequence
  Synthetic
tggcgcagacuuccaaaggcutu

SEQ ID NO: 615 - DNA, 23 bp, Artificial Sequence
  Synthetic
tgggcgcagacuuccaaaggctu

SEQ ID NO: 616 - DNA, 23 bp, Artificial Sequence
  Synthetic
tagggcgcagacuuccaaaggtu

SEQ ID NO: 617 - DNA, 23 bp, Artificial Sequence
  Synthetic
taagggcgcagacuuccaaagtu

SEQ ID NO: 618 - DNA, 23 bp, Artificial Sequence
  Synthetic
tcaagggcgcagacuuccaaatu

SEQ ID NO: 619 - DNA, 23 bp, Artificial Sequence
  Synthetic
tacaagggcgcagacuuccaatu

SEQ ID NO: 620 - DNA, 23 bp, Artificial Sequence
  Synthetic
tcacaagggcgcagacuuccatu

SEQ ID NO: 621 - DNA, 23 bp, Artificial Sequence
  Synthetic
tgcacaagggcgcagacuucctu

SEQ ID NO: 622 - DNA, 23 bp, Artificial Sequence
  Synthetic
tggcacaagggcgcagacuuctu

SEQ ID NO: 623 - DNA, 23 bp, Artificial Sequence
  Synthetic
tgggcacaagggcgcagacuutu

SEQ ID NO: 624 - DNA, 23 bp, Artificial Sequence
  Synthetic
tagggcacaagggcgcagacutu

SEQ ID NO: 625 - DNA, 23 bp, Artificial Sequence
  Synthetic
tcagggcacaagggcgcagactu

SEQ ID NO: 626 - DNA, 23 bp, Artificial Sequence
  Synthetic
tgcagggcacaagggcgcagatu

SEQ ID NO: 627 - DNA, 23 bp, Artificial Sequence
  Synthetic
tggcagggcacaagggcgcagtu

SEQ ID NO: 628 - DNA, 23 bp, Artificial Sequence
  Synthetic
taggcagggcacaagggcgcatu

SEQ ID NO: 629 - DNA, 23 bp, Artificial Sequence
  Synthetic
tgaggcagggcacaagggcgctu

SEQ ID NO: 630 - DNA, 23 bp, Artificial Sequence
  Synthetic
tggaggcagggcacaagggcgtu

SEQ ID NO: 631 - DNA, 23 bp, Artificial Sequence
  Synthetic
tgcagacuuccaaaggcuccgtu

SEQ ID NO: 632 - DNA, 23 bp, Artificial Sequence
  Synthetic
tcgcagacuuccaaaggcucctu

SEQ ID NO: 633 - DNA, 23 bp, Artificial Sequence
  Synthetic
tgcgcagacuuccaaaggcuctu

SEQ ID NO: 634 - DNA, 23 bp, Artificial Sequence
  Synthetic
tggcgcagacuuccaaaggcutu

SEQ ID NO: 635 - DNA, 23 bp, Artificial Sequence
  Synthetic
tgggcgcagacuuccaaaggctu

SEQ ID NO: 636 - DNA, 23 bp, Artificial Sequence
  Synthetic
tagggcgcagacuuccaaaggtu

SEQ ID NO: 637 - DNA, 23 bp, Artificial Sequence
  Synthetic
taagggcgcagacuuccaaagtu

SEQ ID NO: 638 - DNA, 23 bp, Artificial Sequence
  Synthetic
tcaagggcgcagacuuccaaatu

SEQ ID NO: 639 - DNA, 23 bp, Artificial Sequence
  Synthetic
tacaagggcgcagacuuccaatu

SEQ ID NO: 640 - DNA, 23 bp, Artificial Sequence
  Synthetic
tcacaagggcgcagacuuccatu

SEQ ID NO: 641 - DNA, 23 bp, Artificial Sequence
  Synthetic
tgcacaagggcgcagacuucctu

SEQ ID NO: 642 - DNA, 23 bp, Artificial Sequence
  Synthetic
tggcacaagggcgcagacuuctu

SEQ ID NO: 643 - DNA, 23 bp, Artificial Sequence
  Synthetic
tgggcacaagggcgcagacuutu

SEQ ID NO: 644 - DNA, 23 bp, Artificial Sequence
  Synthetic
tagggcacaagggcgcagacutu

SEQ ID NO: 645 - DNA, 23 bp, Artificial Sequence
  Synthetic
tcagggcacaagggcgcagactu

SEQ ID NO: 646 - DNA, 23 bp, Artificial Sequence
  Synthetic
tgcagggcacaagggcgcagatu

SEQ ID NO: 647 - DNA, 23 bp, Artificial Sequence
  Synthetic
tggcagggcacaagggcgcagtu

SEQ ID NO: 648 - DNA, 23 bp, Artificial Sequence
  Synthetic
taggcagggcacaagggcgcatu

SEQ ID NO: 649 - DNA, 23 bp, Artificial Sequence
  Synthetic
tgaggcagggcacaagggcgctu

SEQ ID NO: 650 - DNA, 23 bp, Artificial Sequence
  Synthetic
tggaggcagggcacaagggcgtu

SEQ ID NO: 651 - RNA, 20 nt, Artificial Sequence
  Synthetic
cccucaucuacugugugcac

SEQ ID NO: 652 - DNA, 20 bp, Artificial Sequence
  Synthetic
cctcaggcccccaggttacc

SEQ ID NO: 653 - DNA, 20 bp, Artificial Sequence
  Synthetic
gttgatctgtagcagcagct

SEQ ID NO: 654 - DNA, 20 bp, Artificial Sequence
  Synthetic
gttgatctgtagcagcagct

SEQ ID NO: 655 - DNA, 20 bp, Artificial Sequence
  Synthetic
gttgatctgtagcagcagct

SEQ ID NO: 656 - DNA, 20 bp, Artificial Sequence
  Synthetic
gttgatctgtagcagcagct

SEQ ID NO: 657 - DNA, 20 bp, Artificial Sequence
  Synthetic
gttgatctgtagcagcagct

SEQ ID NO: 658 - DNA, 20 bp, Artificial Sequence
  Synthetic
gttgatctgtagcagcagct

SEQ ID NO: 659 - DNA, 20 bp, Artificial Sequence
  Synthetic
gttgatctgtagcagcagct

SEQ ID NO: 660 - DNA, 20 bp, Artificial Sequence
  Synthetic
gttgatctgtagcagcagct

SEQ ID NO: 661 - DNA, 20 bp, Artificial Sequence
  Synthetic
gttgatctgtagcagcagct

SEQ ID NO: 662 - DNA, 20 bp, Artificial Sequence
  Synthetic
gttgatctgtagcagcagct

SEQ ID NO: 663 - DNA, 20 bp, Artificial Sequence
  Synthetic
gttgatctgtagcaggcagc

SEQ ID NO: 664 - DNA, 20 bp, Artificial Sequence
  Synthetic
gttgatctgtagcaggcagc

SEQ ID NO: 665 - DNA, 20 bp, Artificial Sequence
  Synthetic
gttgatctgtagcaggcagc

SEQ ID NO: 666 - DNA, 20 bp, Artificial Sequence
  Synthetic
gttgatctgtagcaggcagc

SEQ ID NO: 667 - DNA, 20 bp, Artificial Sequence
  Synthetic
gttgatctgtagcaggcagc

SEQ ID NO: 668 - DNA, 20 bp, Artificial Sequence
  Synthetic
gttgatctgtagcaggcagc

SEQ ID NO: 669 - DNA, 20 bp, Artificial Sequence
  Synthetic
gttgatctgtagcaggcagc

SEQ ID NO: 670 - DNA, 20 bp, Artificial Sequence
  Synthetic
gttgatctgtagcaggcagc

SEQ ID NO: 671 - DNA, 20 bp, Artificial Sequence
  Synthetic
gttgatctgtagcaggcagc

SEQ ID NO: 672 - DNA, 20 bp, Artificial Sequence
  Synthetic
gttgatctgtagcaggcagc

SEQ ID NO: 673 - DNA, 20 bp, Artificial Sequence
  Synthetic
guugatctgtagcagcagct

SEQ ID NO: 674 - DNA, 20 bp, Artificial Sequence
  Synthetic
guugatctgtagcagcagct

SEQ ID NO: 675 - DNA, 20 bp, Artificial Sequence
  Synthetic
guugatctgtagcagcagct

SEQ ID NO: 676 - DNA, 20 bp, Artificial Sequence
  Synthetic
guugatctgtagcagcagct

SEQ ID NO: 677 - DNA, 20 bp, Artificial Sequence
  Synthetic
guugatctgtagcagcagct

SEQ ID NO: 678 - DNA, 20 bp, Artificial Sequence
  Synthetic
guugatctgtagcagcagct

SEQ ID NO: 679 - DNA, 20 bp, Artificial Sequence
  Synthetic
guugatctgtagcagcagct

SEQ ID NO: 680 - DNA, 20 bp, Artificial Sequence
  Synthetic
guugatctgtagcagcagct

SEQ ID NO: 681 - DNA, 20 bp, Artificial Sequence
  Synthetic
guugatctgtagcagcagct

SEQ ID NO: 682 - DNA, 20 bp, Artificial Sequence
  Synthetic
guugatctgtagcagcagct

SEQ ID NO: 683 - DNA, 20 bp, Artificial Sequence
  Synthetic
ggcacaagggcgcagacttc

SEQ ID NO: 684 - RNA, 20 nt, Artificial Sequence
  Synthetic
ggcacaagggcgcagacuuc

SEQ ID NO: 685 - RNA, 20 nt, Artificial Sequence
  Synthetic
ggcacaagggcgcagacuuc

SEQ ID NO: 686 - DNA, 20 bp, Artificial Sequence
  Synthetic
ggcacaagggcgcagacttc

SEQ ID NO: 687 - DNA, 20 bp, Artificial Sequence
  Synthetic
ggcacaagggcgcagacttc

SEQ ID NO: 688 - DNA, 20 bp, Artificial Sequence
  Synthetic
ggcacaagggcgcagacttc

SEQ ID NO: 689 - RNA, 20 nt, Artificial Sequence
  Synthetic
ggcacaagggcgcagacuuc

SEQ ID NO: 690 - DNA, 20 bp, Artificial Sequence
  Synthetic
ggcacaagggcgcagacttc

SEQ ID NO: 691 - DNA, 20 bp, Artificial Sequence
  Synthetic
ggcacaagggcgcagacttc

SEQ ID NO: 692 - DNA, 20 bp, Artificial Sequence
  Synthetic
ggcacaagggcgcagacttc

SEQ ID NO: 693 - RNA, 20 nt, Artificial Sequence
  Synthetic
ggcacaagggcgcagacuuc

SEQ ID NO: 694 - RNA, 20 nt, Artificial Sequence
  Synthetic
ggcacaagggcgcagacuuc

SEQ ID NO: 695 - RNA, 20 nt, Artificial Sequence
  Synthetic
ggcacaagggcgcagacuuc

SEQ ID NO: 696 - RNA, 20 nt, Artificial Sequence
  Synthetic
ggcacaagggcgcagacuuc

SEQ ID NO: 697 - RNA, 20 nt, Artificial Sequence
  Synthetic
ggcacaagggcgcagacuuc

SEQ ID NO: 698 - DNA, 20 bp, Artificial Sequence
  Synthetic
gttgatctgtagcagcagct

SEQ ID NO: 699 - DNA, 20 bp, Artificial Sequence
  Synthetic
gttgatctgtagcagcagcu

SEQ ID NO: 700 - DNA, 20 bp, Artificial Sequence
  Synthetic
guugatctgtagcagcagct

SEQ ID NO: 701 - DNA, 20 bp, Artificial Sequence
  Synthetic
guugatctgtagcagcagct

SEQ ID NO: 702 - DNA, 20 bp, Artificial Sequence
  Synthetic
guugatctgtagcagcagct

SEQ ID NO: 703 - DNA, 20 bp, Artificial Sequence
  Synthetic
gttgatctgtagcagcagcu

SEQ ID NO: 704 - DNA, 20 bp, Artificial Sequence
  Synthetic
guugatctgtagcagcagct

SEQ ID NO: 705 - DNA, 20 bp, Artificial Sequence
  Synthetic
guugatctgtagcagcagct

SEQ ID NO: 706 - DNA, 20 bp, Artificial Sequence
  Synthetic
guugatctgtagcagcagct

SEQ ID NO: 707 - DNA, 20 bp, Artificial Sequence
  Synthetic
guugatctgtagcagcagcu

SEQ ID NO: 708 - DNA, 20 bp, Artificial Sequence
  Synthetic
guugatctgtagcagcagcu

SEQ ID NO: 709 - DNA, 20 bp, Artificial Sequence
  Synthetic
guugatctgtagcagcagcu

SEQ ID NO: 710 - DNA, 20 bp, Artificial Sequence
  Synthetic
guugatctgtagcagcagcu

SEQ ID NO: 711 - DNA, 20 bp, Artificial Sequence
  Synthetic
guugatctgtagcagcagcu

SEQ ID NO: 712 - DNA, 20 bp, Artificial Sequence
  Synthetic
gagcggagaaaccctccaaa

SEQ ID NO: 713 - DNA, 20 bp, Artificial Sequence
  Synthetic
tgagcggagaaaccctccaa

SEQ ID NO: 714 - DNA, 20 bp, Artificial Sequence
  Synthetic
ctgagcggagaaaccctcca

SEQ ID NO: 715 - DNA, 20 bp, Artificial Sequence
  Synthetic
gctgagcggagaaaccctcc

SEQ ID NO: 716 - DNA, 20 bp, Artificial Sequence
  Synthetic
ggctgagcggagaaaccctc

SEQ ID NO: 717 - DNA, 20 bp, Artificial Sequence
  Synthetic
aggctgagcggagaaaccct

SEQ ID NO: 718 - DNA, 20 bp, Artificial Sequence
  Synthetic
aaggctgagcggagaaaccc

SEQ ID NO: 719 - DNA, 20 bp, Artificial Sequence
  Synthetic
caaggctgagcggagaaacc

SEQ ID NO: 720 - DNA, 20 bp, Artificial Sequence
  Synthetic
ccaaggctgagcggagaaac

SEQ ID NO: 721 - DNA, 20 bp, Artificial Sequence
  Synthetic
tccaaggctgagcggagaaa

SEQ ID NO: 722 - DNA, 20 bp, Artificial Sequence
  Synthetic
gagcggagaaaccctccaaa

SEQ ID NO: 723 - DNA, 20 bp, Artificial Sequence
  Synthetic
tgagcggagaaacccuccaa

SEQ ID NO: 724 - DNA, 20 bp, Artificial Sequence
  Synthetic
ctgagcggagaaacccucca

SEQ ID NO: 725 - DNA, 20 bp, Artificial Sequence
  Synthetic
gctgagcggagaaacccucc

SEQ ID NO: 726 - DNA, 20 bp, Artificial Sequence
  Synthetic
ggctgagcggagaaacccuc

SEQ ID NO: 727 - DNA, 20 bp, Artificial Sequence
  Synthetic
aggctgagcggagaaacccu

SEQ ID NO: 728 - DNA, 20 bp, Artificial Sequence
  Synthetic
aaggctgagcggagaaaccc

SEQ ID NO: 729 - DNA, 20 bp, Artificial Sequence
  Synthetic
caaggctgagcggagaaacc

SEQ ID NO: 730 - DNA, 20 bp, Artificial Sequence
  Synthetic
ccaaggctgagcggagaaac

SEQ ID NO: 731 - DNA, 20 bp, Artificial Sequence
  Synthetic
tccaaggctgagcggagaaa

SEQ ID NO: 732 - DNA, 20 bp, Artificial Sequence
  Synthetic
gagcggagaaaccctccaaa

SEQ ID NO: 733 - DNA, 20 bp, Artificial Sequence
  Synthetic
ugagcggagaaaccctccaa

SEQ ID NO: 734 - DNA, 20 bp, Artificial Sequence
  Synthetic
cugagcggagaaaccctcca

SEQ ID NO: 735 - DNA, 20 bp, Artificial Sequence
  Synthetic
gcugagcggagaaaccctcc

SEQ ID NO: 736 - DNA, 20 bp, Artificial Sequence
  Synthetic
ggcugagcggagaaaccctc

SEQ ID NO: 737 - DNA, 20 bp, Artificial Sequence
  Synthetic
aggctgagcggagaaaccct

SEQ ID NO: 738 - DNA, 20 bp, Artificial Sequence
  Synthetic
aaggctgagcggagaaaccc

SEQ ID NO: 739 - DNA, 20 bp, Artificial Sequence
  Synthetic
caaggctgagcggagaaacc

SEQ ID NO: 740 - DNA, 20 bp, Artificial Sequence
  Synthetic
ccaaggctgagcggagaaac

SEQ ID NO: 741 - DNA, 20 bp, Artificial Sequence
  Synthetic
uccaaggctgagcggagaaa

SEQ ID NO: 742 - DNA, 20 bp, Artificial Sequence
  Synthetic
gtgcacacagtagatgaggg

SEQ ID NO: 743 - DNA, 20 bp, Artificial Sequence
  Synthetic
gtgcacacagtagatgaggg

SEQ ID NO: 744 - DNA, 20 bp, Artificial Sequence
  Synthetic
gtgcacacagtagatgaggg

SEQ ID NO: 745 - DNA, 20 bp, Artificial Sequence
  Synthetic
gtgcacacagtagatgaggg

SEQ ID NO: 746 - DNA, 20 bp, Artificial Sequence
  Synthetic
gtgcacacagtggatgaggg

SEQ ID NO: 747 - RNA, 20 nt, Artificial Sequence
  Synthetic
ggcacaagggcgcagacuuc

SEQ ID NO: 748 - DNA, 20 bp, Artificial Sequence
  Synthetic
aactcaggccccctaccctg

SEQ ID NO: 749 - DNA, 20 bp, Artificial Sequence
  Synthetic
gaggaggccgtgcagggctc

SEQ ID NO: 750 - DNA, 20 bp, Artificial Sequence
  Synthetic
agaggaggccgtgcagggct

SEQ ID NO: 751 - DNA, 20 bp, Artificial Sequence
  Synthetic
tagaggaggccgtgcagggc

SEQ ID NO: 752 - DNA, 20 bp, Artificial Sequence
  Synthetic
atagaggaggccgtgcaggg

SEQ ID NO: 753 - DNA, 20 bp, Artificial Sequence
  Synthetic
catagaggaggccgtgcagg

SEQ ID NO: 754 - DNA, 20 bp, Artificial Sequence
  Synthetic
acatagaggaggccgtgcag

SEQ ID NO: 755 - DNA, 20 bp, Artificial Sequence
  Synthetic
cacatagaggaggccgtgca

SEQ ID NO: 756 - DNA, 20 bp, Artificial Sequence
  Synthetic
gaggaggccgtgcagggctc

SEQ ID NO: 757 - DNA, 20 bp, Artificial Sequence
  Synthetic
agaggaggccgtgcagggct

SEQ ID NO: 758 - DNA, 20 bp, Artificial Sequence
  Synthetic
tagaggaggccgtgcagggc

SEQ ID NO: 759 - DNA, 20 bp, Artificial Sequence
  Synthetic
atagaggaggccgtgcaggg

SEQ ID NO: 760 - DNA, 20 bp, Artificial Sequence
  Synthetic
catagaggaggccgtgcagg

SEQ ID NO: 761 - DNA, 20 bp, Artificial Sequence
  Synthetic
acatagaggaggccgtgcag

SEQ ID NO: 762 - DNA, 20 bp, Artificial Sequence
  Synthetic
cacatagaggaggccgtgca

SEQ ID NO: 763 - DNA, 20 bp, Artificial Sequence
  Synthetic
gaggaggccgtgcagggcuc

SEQ ID NO: 764 - DNA, 20 bp, Artificial Sequence
  Synthetic
agaggaggccgtgcagggcu

SEQ ID NO: 765 - DNA, 20 bp, Artificial Sequence
  Synthetic
tagaggaggccgtgcagggc

SEQ ID NO: 766 - DNA, 20 bp, Artificial Sequence
  Synthetic
atagaggaggccgtgcaggg

SEQ ID NO: 767 - DNA, 20 bp, Artificial Sequence
  Synthetic
catagaggaggccgtgcagg

SEQ ID NO: 768 - DNA, 20 bp, Artificial Sequence
  Synthetic
acatagaggaggccgugcag

SEQ ID NO: 769 - DNA, 20 bp, Artificial Sequence
  Synthetic
cacatagaggaggccgugca

SEQ ID NO: 770 - DNA, 20 bp, Artificial Sequence
  Synthetic
gaggaggccgtgcagggctc

SEQ ID NO: 771 - DNA, 20 bp, Artificial Sequence
  Synthetic
agaggaggccgtgcagggct

SEQ ID NO: 772 - DNA, 20 bp, Artificial Sequence
  Synthetic
uagaggaggccgtgcagggc

SEQ ID NO: 773 - DNA, 20 bp, Artificial Sequence
  Synthetic
auagaggaggccgtgcaggg

SEQ ID NO: 774 - DNA, 20 bp, Artificial Sequence
  Synthetic
cauagaggaggccgtgcagg

SEQ ID NO: 775 - DNA, 20 bp, Artificial Sequence
  Synthetic
acauagaggaggccgtgcag

SEQ ID NO: 776 - DNA, 20 bp, Artificial Sequence
  Synthetic
cacauagaggaggccgtgca

SEQ ID NO: 777 - DNA, 24 bp, Artificial Sequence
  Synthetic
ggcacaagggcgcagacttccaaa

SEQ ID NO: 778 - DNA, 20 bp, Artificial Sequence
  Synthetic
cacaagggcgcagacttcca

SEQ ID NO: 779 - DNA, 20 bp, Artificial Sequence
  Synthetic
cacaagggcgcagacttcca

SEQ ID NO: 780 - DNA, 20 bp, Artificial Sequence
  Synthetic
cacaagggcgcagacttcca

SEQ ID NO: 781 - DNA, 20 bp, Artificial Sequence
  Synthetic
cacaagggcgcagacttcca

SEQ ID NO: 782 - DNA, 20 bp, Artificial Sequence
  Synthetic
cacaagggcgcagacttcca

SEQ ID NO: 783 - DNA, 20 bp, Artificial Sequence
  Synthetic
cacaagggcgcagacttcca

SEQ ID NO: 784 - DNA, 20 bp, Artificial Sequence
  Synthetic
cacaagggcgcagacttcca

SEQ ID NO: 785 - DNA, 20 bp, Artificial Sequence
  Synthetic
cacaagggcgcagacttcca

SEQ ID NO: 786 - DNA, 20 bp, Artificial Sequence
  Synthetic
cacaagggcgcagacttcca

SEQ ID NO: 787 - DNA, 20 bp, Artificial Sequence
  Synthetic
cacaagggcgcagacttcca

SEQ ID NO: 788 - DNA, 20 bp, Artificial Sequence
  Synthetic
cacaagggcgcagacttcca

SEQ ID NO: 789 - DNA, 20 bp, Artificial Sequence
  Synthetic
cacaagggcgcagacttcca

SEQ ID NO: 790 - DNA, 20 bp, Artificial Sequence
  Synthetic
cacaagggcgcagacttcca

SEQ ID NO: 791 - DNA, 20 bp, Artificial Sequence
  Synthetic
cacaagggcgcagacttcca

SEQ ID NO: 792 - DNA, 20 bp, Artificial Sequence
  Synthetic
cacaagggcgcagacttcca

SEQ ID NO: 793 - DNA, 20 bp, Artificial Sequence
  Synthetic
cacaagggcgcagacttcca

SEQ ID NO: 794 - DNA, 20 bp, Artificial Sequence
  Synthetic
cacaagggcgcagacttcca

SEQ ID NO: 795 - DNA, 20 bp, Artificial Sequence
  Synthetic
cacaagggcgcagacttcca

SEQ ID NO: 796 - DNA, 20 bp, Artificial Sequence
  Synthetic
cacaagggcgcagacttcca

SEQ ID NO: 797 - DNA, 20 bp, Artificial Sequence
  Synthetic
cacaagggcgcagacttcca

SEQ ID NO: 798 - DNA, 20 bp, Artificial Sequence
  Synthetic
cacaagggcacagacttcca

SEQ ID NO: 799 - DNA, 20 bp, Artificial Sequence
  Synthetic
cacaagggcacagacttcca

SEQ ID NO: 800 - DNA, 20 bp, Artificial Sequence
  Synthetic
cacaagggcacagacttcca

SEQ ID NO: 801 - DNA, 20 bp, Artificial Sequence
  Synthetic
cacaagggcacagacttcca

SEQ ID NO: 802 - DNA, 20 bp, Artificial Sequence
  Synthetic
cacaagggcacagacttcca

SEQ ID NO: 803 - DNA, 20 bp, Artificial Sequence
  Synthetic
cacaagggcacagacttcca

SEQ ID NO: 804 - DNA, 20 bp, Artificial Sequence
  Synthetic
cacaagggcacagacttcca

SEQ ID NO: 805 - DNA, 20 bp, Artificial Sequence
  Synthetic
cacaagggcacagacttcca

SEQ ID NO: 806 - DNA, 20 bp, Artificial Sequence
  Synthetic
cacaagggcacagacttcca

SEQ ID NO: 807 - DNA, 20 bp, Artificial Sequence
  Synthetic
cacaagggcacagacttcca

SEQ ID NO: 808 - DNA, 20 bp, Artificial Sequence
  Synthetic
cacaagggcacagacttcca

SEQ ID NO: 809 - DNA, 20 bp, Artificial Sequence
  Synthetic
cacaagggcacagacttcca

SEQ ID NO: 810 - DNA, 20 bp, Artificial Sequence
  Synthetic
cacaagggcacagacttcca

SEQ ID NO: 811 - DNA, 20 bp, Artificial Sequence
  Synthetic
cacaagggcacagacttcca

SEQ ID NO: 812 - DNA, 20 bp, Artificial Sequence
  Synthetic
cacaagggcacagacttcca

SEQ ID NO: 813 - DNA, 20 bp, Artificial Sequence
  Synthetic
cacaagggcacagacttcca

SEQ ID NO: 814 - DNA, 20 bp, Artificial Sequence
  Synthetic
cacaagggcacagacttcca

SEQ ID NO: 815 - DNA, 20 bp, Artificial Sequence
  Synthetic
cacaagggcacagacttcca

SEQ ID NO: 816 - DNA, 20 bp, Artificial Sequence
  Synthetic
cacaagggcacagacttcca

SEQ ID NO: 817 - DNA, 20 bp, Artificial Sequence
  Synthetic
cacaagggcacagacttcca

SEQ ID NO: 818 - DNA, 20 bp, Artificial Sequence
  Synthetic
cacaagggcacagacttcca

SEQ ID NO: 819 - DNA, 24 bp, Artificial Sequence
  Synthetic
ggcacaagggcgcagacttccaaa

SEQ ID NO: 820 - DNA, 20 bp, Artificial Sequence
  Synthetic
gttaccgccatccccgccgu

SEQ ID NO: 821 - DNA, 20 bp, Artificial Sequence
  Synthetic
gttaccgccatccccgccgu

SEQ ID NO: 822 - DNA, 20 bp, Artificial Sequence
  Synthetic
gttaccgccatccccgccgu

SEQ ID NO: 823 - DNA, 20 bp, Artificial Sequence
  Synthetic
gttaccgccatccccgccgu

SEQ ID NO: 824 - DNA, 20 bp, Artificial Sequence
  Synthetic
gttaccgccatccccgccgu

SEQ ID NO: 825 - DNA, 20 bp, Artificial Sequence
  Synthetic
gttaccgccatccccgccgu

SEQ ID NO: 826 - DNA, 20 bp, Artificial Sequence
  Synthetic
gttaccgccatccccgccgu

SEQ ID NO: 827 - DNA, 20 bp, Artificial Sequence
  Synthetic
gttaccgccatccccgccgu

SEQ ID NO: 828 - DNA, 20 bp, Artificial Sequence
  Synthetic
gttaccgccatccccgccgu

SEQ ID NO: 829 - DNA, 20 bp, Artificial Sequence
  Synthetic
gttaccgccatccccgccgu

SEQ ID NO: 830 - DNA, 20 bp, Artificial Sequence
  Synthetic
gttaccgccatccccgccgt

SEQ ID NO: 831 - DNA, 20 bp, Artificial Sequence
  Synthetic
gttaccgccatccccgccgt

SEQ ID NO: 832 - DNA, 20 bp, Artificial Sequence
  Synthetic
gttaccgccatccccgccgt

SEQ ID NO: 833 - DNA, 20 bp, Artificial Sequence
  Synthetic
gttaccgccatccccgccgt

SEQ ID NO: 834 - DNA, 20 bp, Artificial Sequence
  Synthetic
gttaccgccatccccgccgt

SEQ ID NO: 835 - DNA, 20 bp, Artificial Sequence
  Synthetic
gttaccgccatccccgccgt

SEQ ID NO: 836 - DNA, 20 bp, Artificial Sequence
  Synthetic
gttaccgccatccccgccgt

SEQ ID NO: 837 - DNA, 20 bp, Artificial Sequence
  Synthetic
gttaccgccatccccgccgt

SEQ ID NO: 838 - DNA, 20 bp, Artificial Sequence
  Synthetic
gttaccgccatccccgccgt

SEQ ID NO: 839 - DNA, 20 bp, Artificial Sequence
  Synthetic
gttaccgccatccccgccgt

SEQ ID NO: 840 - DNA, 20 bp, Artificial Sequence
  Synthetic
accgccatccccgccguagc

SEQ ID NO: 841 - DNA, 20 bp, Artificial Sequence
  Synthetic
accgccatccccgccguagc

SEQ ID NO: 842 - DNA, 20 bp, Artificial Sequence
  Synthetic
accgccatccccgccguagc

SEQ ID NO: 843 - DNA, 20 bp, Artificial Sequence
  Synthetic
accgccatccccgccguagc

SEQ ID NO: 844 - DNA, 20 bp, Artificial Sequence
  Synthetic
accgccatccccgccguagc

SEQ ID NO: 845 - DNA, 20 bp, Artificial Sequence
  Synthetic
accgccatccccgccguagc

SEQ ID NO: 846 - DNA, 20 bp, Artificial Sequence
  Synthetic
accgccatccccgccguagc

SEQ ID NO: 847 - DNA, 20 bp, Artificial Sequence
  Synthetic
accgccatccccgccguagc

SEQ ID NO: 848 - DNA, 20 bp, Artificial Sequence
  Synthetic
accgccatccccgccguagc

SEQ ID NO: 849 - DNA, 20 bp, Artificial Sequence
  Synthetic
accgccatccccgccguagc

SEQ ID NO: 850 - DNA, 20 bp, Artificial Sequence
  Synthetic
accgccatccccgccgtagc

SEQ ID NO: 851 - DNA, 20 bp, Artificial Sequence
  Synthetic
accgccatccccgccgtagc

SEQ ID NO: 852 - DNA, 20 bp, Artificial Sequence
  Synthetic
accgccatccccgccgtagc

SEQ ID NO: 853 - DNA, 20 bp, Artificial Sequence
  Synthetic
accgccatccccgccgtagc

SEQ ID NO: 854 - DNA, 20 bp, Artificial Sequence
  Synthetic
accgccatccccgccgtagc

SEQ ID NO: 855 - DNA, 20 bp, Artificial Sequence
  Synthetic
accgccatccccgccgtagc

SEQ ID NO: 856 - DNA, 20 bp, Artificial Sequence
  Synthetic
accgccatccccgccgtagc

SEQ ID NO: 857 - DNA, 20 bp, Artificial Sequence
  Synthetic
accgccatccccgccgtagc

SEQ ID NO: 858 - DNA, 20 bp, Artificial Sequence
  Synthetic
accgccatccccgccgtagc

SEQ ID NO: 859 - DNA, 20 bp, Artificial Sequence
  Synthetic
accgccatccccgccgtagc

SEQ ID NO: 860 - DNA, 20 bp, Artificial Sequence
  Synthetic
caagggcgcagacttccaaa

SEQ ID NO: 861 - DNA, 20 bp, Artificial Sequence
  Synthetic
acaagggcgcagacttccaa

SEQ ID NO: 862 - DNA, 20 bp, Artificial Sequence
  Synthetic
cacaagggcgcagactucca

SEQ ID NO: 863 - RNA, 20 nt, Artificial Sequence
  Synthetic
gcacaagggcgcagacuucc

SEQ ID NO: 864 - RNA, 20 nt, Artificial Sequence
  Synthetic
ggcacaagggcgcagacuuc

SEQ ID NO: 865 - DNA, 20 bp, Artificial Sequence
  Synthetic
caagggcgcagacttccaaa

SEQ ID NO: 866 - DNA, 20 bp, Artificial Sequence
  Synthetic
acaagggcgcagacttccaa

SEQ ID NO: 867 - DNA, 20 bp, Artificial Sequence
  Synthetic
cacaagggcgcagacttcca

SEQ ID NO: 868 - DNA, 20 bp, Artificial Sequence
  Synthetic
gcacaagggcgcagacttcc

SEQ ID NO: 869 - DNA, 20 bp, Artificial Sequence
  Synthetic
ggcacaagggcgcagacttc

SEQ ID NO: 870 - DNA, 20 bp, Artificial Sequence
  Synthetic
caagggcgcagacttccaaa

SEQ ID NO: 871 - DNA, 20 bp, Artificial Sequence
  Synthetic
acatagaggaggccgtgcag

SEQ ID NO: 872 - DNA, 20 bp, Artificial Sequence
  Synthetic
cacaagggcgcagacttcca

SEQ ID NO: 873 - DNA, 20 bp, Artificial Sequence
  Synthetic
gcacaagggcgcagacttcc

SEQ ID NO: 874 - DNA, 20 bp, Artificial Sequence
  Synthetic
ggcacaagggcgcagacttc

SEQ ID NO: 875 - DNA, 20 bp, Artificial Sequence
  Synthetic
caagggcacagacttccaaa

SEQ ID NO: 876 - DNA, 20 bp, Artificial Sequence
  Synthetic
acaagggcacagacttccaa

SEQ ID NO: 877 - DNA, 20 bp, Artificial Sequence
  Synthetic
cacaagggcacagactucca

SEQ ID NO: 878 - RNA, 20 nt, Artificial Sequence
  Synthetic
gcacaagggcacagacuucc

SEQ ID NO: 879 - RNA, 20 nt, Artificial Sequence
  Synthetic
ggcacaagggcacagacuuc

SEQ ID NO: 880 - DNA, 20 bp, Artificial Sequence
  Synthetic
caagggcacagacttccaaa

SEQ ID NO: 881 - DNA, 20 bp, Artificial Sequence
  Synthetic
acaagggcacagacttccaa

SEQ ID NO: 882 - DNA, 20 bp, Artificial Sequence
  Synthetic
cacaagggcacagacttcca

SEQ ID NO: 883 - DNA, 20 bp, Artificial Sequence
  Synthetic
gcacaagggcacagacttcc

SEQ ID NO: 884 - DNA, 20 bp, Artificial Sequence
  Synthetic
ggcacaagggcacagacttc

SEQ ID NO: 885 - DNA, 20 bp, Artificial Sequence
  Synthetic
caagggcacagacttccaaa

SEQ ID NO: 886 - DNA, 20 bp, Artificial Sequence
  Synthetic
acaagggcacagacttccaa

SEQ ID NO: 887 - DNA, 20 bp, Artificial Sequence
  Synthetic
cacaagggcacagacttcca

SEQ ID NO: 888 - DNA, 20 bp, Artificial Sequence
  Synthetic
gcacaagggcacagacttcc

SEQ ID NO: 889 - DNA, 20 bp, Artificial Sequence
  Synthetic
ggcacaagggcacagacttc

SEQ ID NO: 890 - DNA, 20 bp, Artificial Sequence
  Synthetic
acaagggcgcagacttccaa

SEQ ID NO: 891 - DNA, 20 bp, Artificial Sequence
  Synthetic
cacaagggcgcagactucca

SEQ ID NO: 892 - DNA, 20 bp, Artificial Sequence
  Synthetic
cacaagggcgcagactucca

SEQ ID NO: 893 - DNA, 20 bp, Artificial Sequence
  Synthetic
cacaagggcacagactucca

SEQ ID NO: 894 - DNA, 20 bp, Artificial Sequence
  Synthetic
cacaagggcacagactucca

SEQ ID NO: 895 - DNA, 20 bp, Artificial Sequence
  Synthetic
cacaagggcgcagacttcca

SEQ ID NO: 896 - DNA, 20 bp, Artificial Sequence
  Synthetic
cacaagggcgcagacttcca

SEQ ID NO: 897 - DNA, 20 bp, Artificial Sequence
  Synthetic
cacaagggcacagacttcca

SEQ ID NO: 898 - DNA, 20 bp, Artificial Sequence
  Synthetic
cacaagggcacagacttcca

SEQ ID NO: 899 - DNA, 20 bp, Artificial Sequence
  Synthetic
cacaagggcgcagacttcca

SEQ ID NO: 900 - DNA, 20 bp, Artificial Sequence
  Synthetic
cacaagggcgcagacttcca

SEQ ID NO: 901 - DNA, 20 bp, Artificial Sequence
  Synthetic
cacaagggcacagacttcca

SEQ ID NO: 902 - DNA, 20 bp, Artificial Sequence
  Synthetic
cacaagggcacagacttcca

SEQ ID NO: 903 - DNA, 20 bp, Artificial Sequence
  Synthetic
atctgtagcagcagcttctc

SEQ ID NO: 904 - DNA, 20 bp, Artificial Sequence
  Synthetic
gatctgtagcagcagcttct

SEQ ID NO: 905 - DNA, 20 bp, Artificial Sequence
  Synthetic
tgatctgtagcagcagcttc

SEQ ID NO: 906 - DNA, 20 bp, Artificial Sequence
  Synthetic
ttgatctgtagcagcagctt

SEQ ID NO: 907 - DNA, 20 bp, Artificial Sequence
  Synthetic
gttgatctgtagcagcagct

SEQ ID NO: 908 - DNA, 20 bp, Artificial Sequence
  Synthetic
ggttgatctgtagcagcagc

SEQ ID NO: 909 - DNA, 20 bp, Artificial Sequence
  Synthetic
gggttgatctgtagcagcag

SEQ ID NO: 910 - DNA, 20 bp, Artificial Sequence
  Synthetic
atctgtagcagcagcttctc

SEQ ID NO: 911 - DNA, 20 bp, Artificial Sequence
  Synthetic
gatctgtagcagcagcttct

SEQ ID NO: 912 - DNA, 20 bp, Artificial Sequence
  Synthetic
tgatctgtagcagcagcttc

SEQ ID NO: 913 - DNA, 20 bp, Artificial Sequence
  Synthetic
ttgatctgtagcagcagctt

SEQ ID NO: 914 - DNA, 20 bp, Artificial Sequence
  Synthetic
gttgatctgtagcagcagct

SEQ ID NO: 915 - DNA, 20 bp, Artificial Sequence
  Synthetic
ggttgatctgtagcagcagc

SEQ ID NO: 916 - DNA, 20 bp, Artificial Sequence
  Synthetic
gggttgatctgtagcagcag

SEQ ID NO: 917 - DNA, 20 bp, Artificial Sequence
  Synthetic
atctgtagcagcagcuucuc

SEQ ID NO: 918 - DNA, 20 bp, Artificial Sequence
  Synthetic
gatctgtagcagcagcuucu

SEQ ID NO: 919 - DNA, 20 bp, Artificial Sequence
  Synthetic
tgatctgtagcagcagcuuc

SEQ ID NO: 920 - DNA, 20 bp, Artificial Sequence
  Synthetic
ttgatctgtagcagcagcuu

SEQ ID NO: 921 - DNA, 20 bp, Artificial Sequence
  Synthetic
gttgatctgtagcagcagcu

SEQ ID NO: 922 - DNA, 20 bp, Artificial Sequence
  Synthetic
ggttgatctgtagcagcagc

SEQ ID NO: 923 - DNA, 20 bp, Artificial Sequence
  Synthetic
gggttgatctgtagcagcag

SEQ ID NO: 924 - DNA, 20 bp, Artificial Sequence
  Synthetic
atctgtagcagcagctucuc

SEQ ID NO: 925 - DNA, 20 bp, Artificial Sequence
  Synthetic
gatctgtagcagcagcuucu

SEQ ID NO: 926 - DNA, 20 bp, Artificial Sequence
  Synthetic
tgatctgtagcagcagcuuc

SEQ ID NO: 927 - DNA, 20 bp, Artificial Sequence
  Synthetic
ttgatctgtagcagcagcuu

SEQ ID NO: 928 - DNA, 20 bp, Artificial Sequence
  Synthetic
gttgatctgtagcagcagcu

SEQ ID NO: 929 - DNA, 20 bp, Artificial Sequence
  Synthetic
ggttgatctgtagcagcagc

SEQ ID NO: 930 - DNA, 20 bp, Artificial Sequence
  Synthetic
gggttgatctgtagcagcag

SEQ ID NO: 931 - DNA, 20 bp, Artificial Sequence
  Synthetic
gttgatctgtagcagcagct

SEQ ID NO: 932 - DNA, 20 bp, Artificial Sequence
  Synthetic
gttgatctgtagcagcagct

SEQ ID NO: 933 - DNA, 20 bp, Artificial Sequence
  Synthetic
gttgatctgtagcagcagct

SEQ ID NO: 934 - DNA, 20 bp, Artificial Sequence
  Synthetic
gttgatctgtagcagcagct

SEQ ID NO: 935 - DNA, 20 bp, Artificial Sequence
  Synthetic
gttgatctgtagcagcagcu

SEQ ID NO: 936 - DNA, 20 bp, Artificial Sequence
  Synthetic
gttgatctgtagcagcagcu

SEQ ID NO: 937 - DNA, 20 bp, Artificial Sequence
  Synthetic
guugatctgtagcagcagct

SEQ ID NO: 938 - DNA, 20 bp, Artificial Sequence
  Synthetic
guugatctgtagcagcagct

SEQ ID NO: 939 - DNA, 20 bp, Artificial Sequence
  Synthetic
guugatctgtagcagcagct

SEQ ID NO: 940 - DNA, 20 bp, Artificial Sequence
  Synthetic
guugatctgtagcagcagct

SEQ ID NO: 941 - RNA, 20 nt, Artificial Sequence
  Synthetic
ggcacaagggcacagacuuc

SEQ ID NO: 942 - RNA, 20 nt, Artificial Sequence
  Synthetic
ggcacaagggcacagacuuc

SEQ ID NO: 943 - RNA, 20 nt, Artificial Sequence
  Synthetic
ggcacaagggcacagacuuc

SEQ ID NO: 944 - RNA, 20 nt, Artificial Sequence
  Synthetic
ggcacaagggcacagacuuc

SEQ ID NO: 945 - DNA, 20 bp, Artificial Sequence
  Synthetic
gugcacacagtagatgaggg

SEQ ID NO: 946 - DNA, 20 bp, Artificial Sequence
  Synthetic
gugcacacagtagatgaggg

SEQ ID NO: 947 - DNA, 20 bp, Artificial Sequence
  Synthetic
gugcacacagtagatgaggg

SEQ ID NO: 948 - DNA, 20 bp, Artificial Sequence
  Synthetic
gugcacacagtagatgaggg

SEQ ID NO: 949 - DNA, 20 bp, Artificial Sequence
  Synthetic
tcctcccttccctatgtacg

SEQ ID NO: 950 - RNA, 29 nt, Artificial Sequence
  Synthetic
gagaagcugcugcuacagaucaaccccga

SEQ ID NO: 951 - RNA, 29 nt, Artificial Sequence
  Synthetic
gagaagcugcugcugcagaucaaccccga

SEQ ID NO: 952 - DNA, 20 bp, Artificial Sequence
  Synthetic
atctgtagcagcagcttctc

SEQ ID NO: 953 - DNA, 20 bp, Artificial Sequence
  Synthetic
guugatctgtagcagcagct

SEQ ID NO: 954 - DNA, 20 bp, Artificial Sequence
  Synthetic
guugatctgtagcagcagct

SEQ ID NO: 955 - DNA, 20 bp, Artificial Sequence
  Synthetic
guugatctgtagcagcagct

SEQ ID NO: 956 - DNA, 20 bp, Artificial Sequence
  Synthetic
guugatctgtagcagcagct

SEQ ID NO: 957 - DNA, 20 bp, Artificial Sequence
  Synthetic
guugatctgtagcagcagct

SEQ ID NO: 958 - DNA, 20 bp, Artificial Sequence
  Synthetic
guugatctgtagcagcagct

SEQ ID NO: 959 - DNA, 20 bp, Artificial Sequence
  Synthetic
gcacacagtagatgagggag

SEQ ID NO: 960 - DNA, 20 bp, Artificial Sequence
  Synthetic
gcacacagtagatgagggag

SEQ ID NO: 961 - DNA, 20 bp, Artificial Sequence
  Synthetic
gcacacagtagatgagggag

SEQ ID NO: 962 - DNA, 20 bp, Artificial Sequence
  Synthetic
gcacacagtagatgagggag

SEQ ID NO: 963 - DNA, 20 bp, Artificial Sequence
  Synthetic
gcacacagtagatgagggag

SEQ ID NO: 964 - DNA, 20 bp, Artificial Sequence
  Synthetic
gcacacagtagatgagggag

SEQ ID NO: 965 - DNA, 20 bp, Artificial Sequence
  Synthetic
gcacacagtagatgagggag

SEQ ID NO: 966 - DNA, 20 bp, Artificial Sequence
  Synthetic
gcacacagtagatgagggag

SEQ ID NO: 967 - DNA, 20 bp, Artificial Sequence
  Synthetic
gcacacagtagatgagggag

SEQ ID NO: 968 - DNA, 20 bp, Artificial Sequence
  Synthetic
gcacacagtagatgagggag

SEQ ID NO: 969 - DNA, 20 bp, Artificial Sequence
  Synthetic
catatcccctcctctgccag

SEQ ID NO: 970 - RNA, 31 nt, Artificial Sequence
  Synthetic
gccuuuggaagucugugcccuugugcccugc

SEQ ID NO: 971 - DNA, 20 bp, Artificial Sequence
  Synthetic
cacaagggcgcagacttcca

SEQ ID NO: 972 - DNA, 20 bp, Artificial Sequence
  Synthetic
cacaagggcgcagacttcca

SEQ ID NO: 973 - DNA, 20 bp, Artificial Sequence
  Synthetic
cacaagggcgcagacttcca

SEQ ID NO: 974 - DNA, 20 bp, Artificial Sequence
  Synthetic
cacaagggcgcagacttcca

SEQ ID NO: 975 - DNA, 20 bp, Artificial Sequence
  Synthetic
cacaagggcacagacttcca

SEQ ID NO: 976 - DNA, 20 bp, Artificial Sequence
  Synthetic
cacaagggcacagacttcca

SEQ ID NO: 977 - DNA, 20 bp, Artificial Sequence
  Synthetic
cacaagggcacagacttcca

SEQ ID NO: 978 - DNA, 20 bp, Artificial Sequence
  Synthetic
cacaagggcacagacttcca

SEQ ID NO: 979 - RNA, 20 nt, Artificial Sequence
  Synthetic
cacaagggcgcagacuucca

SEQ ID NO: 980 - RNA, 20 nt, Artificial Sequence
  Synthetic
cacaagggcgcagacuucca

SEQ ID NO: 981 - RNA, 20 nt, Artificial Sequence
  Synthetic
cacaagggcgcagacuucca

SEQ ID NO: 982 - RNA, 20 nt, Artificial Sequence
  Synthetic
cacaagggcgcagacuucca

SEQ ID NO: 983 - RNA, 20 nt, Artificial Sequence
  Synthetic
cacaagggcacagacuucca

SEQ ID NO: 984 - RNA, 20 nt, Artificial Sequence
  Synthetic
cacaagggcacagacuucca

SEQ ID NO: 985 - RNA, 20 nt, Artificial Sequence
  Synthetic
cacaagggcacagacuucca

SEQ ID NO: 986 - RNA, 20 nt, Artificial Sequence
  Synthetic
cacaagggcacagacuucca

SEQ ID NO: 987 - DNA, 5 bp, Artificial Sequence
  Synthetic
cgcca

SEQ ID NO: 988 - DNA, 20 bp, Artificial Sequence
  Synthetic
guugatctgtagcagcagct

SEQ ID NO: 989 - DNA, 20 bp, Artificial Sequence
  Synthetic
guugatctgtagcagcagct

SEQ ID NO: 990 - DNA, 20 bp, Artificial Sequence
  Synthetic
guugatctgtagcagcagct

SEQ ID NO: 991 - DNA, 20 bp, Artificial Sequence
  Synthetic
guugatctgtagcagcagct

SEQ ID NO: 992 - DNA, 20 bp, Artificial Sequence
  Synthetic
guugatctgtagcagcagct

SEQ ID NO: 993 - DNA, 20 bp, Artificial Sequence
  Synthetic
ccuucttcattcttgcccaa

SEQ ID NO: 994 - DNA, 20 bp, Artificial Sequence
  Synthetic
guugatctgtagcagcagct

SEQ ID NO: 995 - DNA, 20 bp, Artificial Sequence
  Synthetic
guugatctgtagcagcagct

SEQ ID NO: 996 - DNA, 20 bp, Artificial Sequence
  Synthetic
guugatctgtagcagcagct

SEQ ID NO: 997 - DNA, 20 bp, Artificial Sequence
  Synthetic
guugatctgtagcagcagct

SEQ ID NO: 998 - DNA, 20 bp, Artificial Sequence
  Synthetic
guugatctgtagcagcagct

SEQ ID NO: 999 - DNA, 20 bp, Artificial Sequence
  Synthetic
guugatctgtagcagcagct

SEQ ID NO: 1000 - DNA, 20 bp, Artificial Sequence
  Synthetic
guugatctgtagcagcagct

SEQ ID NO: 1001 - DNA, 20 bp, Artificial Sequence
  Synthetic
gttgatctgtagcagcagcu

SEQ ID NO: 1002 - DNA, 20 bp, Artificial Sequence
  Synthetic
gttgatctgtagcagcagcu

SEQ ID NO: 1003 - DNA, 20 bp, Artificial Sequence
  Synthetic
gttgatctgtagcagcagcu

SEQ ID NO: 1004 - DNA, 20 bp, Artificial Sequence
  Synthetic
gttgatctgtagcagcagcu

SEQ ID NO: 1005 - DNA, 20 bp, Artificial Sequence
  Synthetic
gttgatctgtagcagcagcu

SEQ ID NO: 1006 - DNA, 20 bp, Artificial Sequence
  Synthetic
gttgatctgtagcagcagcu

SEQ ID NO: 1007 - DNA, 20 bp, Artificial Sequence
  Synthetic
gttgatctgtagcagcagcu

SEQ ID NO: 1008 - DNA, 20 bp, Artificial Sequence
  Synthetic
gttgatctgtagcagcagcu

SEQ ID NO: 1009 - DNA, 20 bp, Artificial Sequence
  Synthetic
gttgatctgtagcagcagcu

SEQ ID NO: 1010 - DNA, 20 bp, Artificial Sequence
  Synthetic
gttgatctgtagcagcagcu

SEQ ID NO: 1011 - DNA, 20 bp, Artificial Sequence
  Synthetic
gttgatctgtagcagcagcu

SEQ ID NO: 1012 - DNA, 20 bp, Artificial Sequence
  Synthetic
gttgatctgtagcagcagcu

SEQ ID NO: 1013 - DNA, 20 bp, Artificial Sequence
  Synthetic
gttgatctgtagcagcagcu

SEQ ID NO: 1014 - DNA, 20 bp, Artificial Sequence
  Synthetic
gttgatctgtagcagcagcu

SEQ ID NO: 1015 - DNA, 20 bp, Artificial Sequence
  Synthetic
gttgatctgtagcagcagcu

SEQ ID NO: 1016 - DNA, 20 bp, Artificial Sequence
  Synthetic
gttgatctgtagcagcagcu

SEQ ID NO: 1017 - DNA, 20 bp, Artificial Sequence
  Synthetic
gttgatctgtagcagcagcu

SEQ ID NO: 1018 - DNA, 20 bp, Artificial Sequence
  Synthetic
gttgatctgtagcagcagcu

SEQ ID NO: 1019 - DNA, 20 bp, Artificial Sequence
  Synthetic
caagggcgcagacttccaaa

SEQ ID NO: 1020 - RNA, 20 nt, Artificial Sequence
  Synthetic
cacaagggcgcagacuucca

SEQ ID NO: 1021 - RNA, 20 nt, Artificial Sequence
  Synthetic
ggcacaagggcgcagacuuc

SEQ ID NO: 1022 - DNA, 20 bp, Artificial Sequence
  Synthetic
caagggcgcagacttccaaa

SEQ ID NO: 1023 - RNA, 20 nt, Artificial Sequence
  Synthetic
cacaagggcgcagacuucca

SEQ ID NO: 1024 - RNA, 20 nt, Artificial Sequence
  Synthetic
ggcacaagggcgcagacuuc

SEQ ID NO: 1025 - DNA, 20 bp, Artificial Sequence
  Synthetic
caagggcgcagacttccaaa

SEQ ID NO: 1026 - RNA, 20 nt, Artificial Sequence
  Synthetic
cacaagggcgcagacuucca

SEQ ID NO: 1027 - RNA, 20 nt, Artificial Sequence
  Synthetic
ggcacaagggcgcagacuuc

SEQ ID NO: 1028 - DNA, 20 bp, Artificial Sequence
  Synthetic
caagggcacagacttccaaa

SEQ ID NO: 1029 - DNA, 20 bp, Artificial Sequence
  Synthetic
caagggcacagacttccaaa

SEQ ID NO: 1030 - DNA, 20 bp, Artificial Sequence
  Synthetic
caagggcacagacttccaaa

SEQ ID NO: 1031 - DNA, 20 bp, Artificial Sequence
  Synthetic
caagggcacagacttccaaa

SEQ ID NO: 1032 - DNA, 20 bp, Artificial Sequence
  Synthetic
acaagggcacagactuccaa

SEQ ID NO: 1033 - DNA, 20 bp, Artificial Sequence
  Synthetic
acaagggcacagactuccaa

SEQ ID NO: 1034 - DNA, 20 bp, Artificial Sequence
  Synthetic
acaagggcacagactuccaa

SEQ ID NO: 1035 - DNA, 20 bp, Artificial Sequence
  Synthetic
acaagggcacagactuccaa

SEQ ID NO: 1036 - DNA, 20 bp, Artificial Sequence
  Synthetic
acaagggcacagactuccaa

SEQ ID NO: 1037 - RNA, 20 nt, Artificial Sequence
  Synthetic
cacaagggcacagacuucca

SEQ ID NO: 1038 - RNA, 20 nt, Artificial Sequence
  Synthetic
cacaagggcacagacuucca

SEQ ID NO: 1039 - RNA, 20 nt, Artificial Sequence
  Synthetic
cacaagggcacagacuucca

SEQ ID NO: 1040 - RNA, 20 nt, Artificial Sequence
  Synthetic
cacaagggcacagacuucca

SEQ ID NO: 1041 - RNA, 20 nt, Artificial Sequence
  Synthetic
cacaagggcacagacuucca

SEQ ID NO: 1042 - RNA, 20 nt, Artificial Sequence
  Synthetic
cacaagggcacagacuucca

SEQ ID NO: 1043 - RNA, 20 nt, Artificial Sequence
  Synthetic
gcacaagggcacagacuucc

SEQ ID NO: 1044 - RNA, 20 nt, Artificial Sequence
  Synthetic
gcacaagggcacagacuucc

SEQ ID NO: 1045 - RNA, 20 nt, Artificial Sequence
  Synthetic
gcacaagggcacagacuucc

SEQ ID NO: 1046 - RNA, 20 nt, Artificial Sequence
  Synthetic
gcacaagggcacagacuucc

SEQ ID NO: 1047 - RNA, 20 nt, Artificial Sequence
  Synthetic
gcacaagggcacagacuucc

SEQ ID NO: 1048 - RNA, 20 nt, Artificial Sequence
  Synthetic
ggcacaagggcacagacuuc

SEQ ID NO: 1049 - RNA, 20 nt, Artificial Sequence
  Synthetic
ggcacaagggcacagacuuc

SEQ ID NO: 1050 - RNA, 20 nt, Artificial Sequence
  Synthetic
ggcacaagggcacagacuuc

SEQ ID NO: 1051 - RNA, 20 nt, Artificial Sequence
  Synthetic
ggcacaagggcacagacuuc

SEQ ID NO: 1052 - DNA, 20 bp, Artificial Sequence
  Synthetic
guccucaatgccccagggtt

SEQ ID NO: 1053 - DNA, 20 bp, Artificial Sequence
  Synthetic
cagtttgggcattttgtccg

SEQ ID NO: 1054 - DNA, 20 bp, Artificial Sequence
  Synthetic
guugatctgtagcagcagct

SEQ ID NO: 1055 - DNA, 20 bp, Artificial Sequence
  Synthetic
guugatctgtagcagcagct

SEQ ID NO: 1056 - DNA, 20 bp, Artificial Sequence
  Synthetic
guugatctgtagcagcagct

SEQ ID NO: 1057 - DNA, 20 bp, Artificial Sequence
  Synthetic
guugatctgtagcagcagct

SEQ ID NO: 1058 - DNA, 20 bp, Artificial Sequence
  Synthetic
guugatctgtagcagcagct

SEQ ID NO: 1059 - DNA, 20 bp, Artificial Sequence
  Synthetic
guugatctgtagcagcagct

SEQ ID NO: 1060 - DNA, 20 bp, Artificial Sequence
  Synthetic
guugatctgtagcagcagct

SEQ ID NO: 1061 - DNA, 20 bp, Artificial Sequence
  Synthetic
guugatctgtagcagcagct

SEQ ID NO: 1062 - DNA, 20 bp, Artificial Sequence
  Synthetic
guugatctgtagcagcagct

SEQ ID NO: 1063 - DNA, 20 bp, Artificial Sequence
  Synthetic
guugatctgtagcagcagct

SEQ ID NO: 1064 - DNA, 20 bp, Artificial Sequence
  Synthetic
guugatctgtagcagcagct

SEQ ID NO: 1065 - DNA, 20 bp, Artificial Sequence
  Synthetic
guugatctgtagcagcagct

SEQ ID NO: 1066 - DNA, 20 bp, Artificial Sequence
  Synthetic
guugatctgtagcagcagct

SEQ ID NO: 1067 - DNA, 20 bp, Artificial Sequence
  Synthetic
guugatctgtagcagcagct

SEQ ID NO: 1068 - DNA, 20 bp, Artificial Sequence
  Synthetic
guugatctgtagcagcagct

SEQ ID NO: 1069 - DNA, 20 bp, Artificial Sequence
  Synthetic
guugatctgtagcagcagct

SEQ ID NO: 1070 - DNA, 20 bp, Artificial Sequence
  Synthetic
guugatctgtagcagcagct

SEQ ID NO: 1071 - DNA, 20 bp, Artificial Sequence
  Synthetic
guugatctgtagcagcagct

SEQ ID NO: 1072 - DNA, 20 bp, Artificial Sequence
  Synthetic
guugatctgtagcagcagct

SEQ ID NO: 1073 - DNA, 20 bp, Artificial Sequence
  Synthetic
gttgatctgtagcagcagcu

SEQ ID NO: 1074 - DNA, 20 bp, Artificial Sequence
  Synthetic
gttgatctgtagcagcagcu

SEQ ID NO: 1075 - DNA, 20 bp, Artificial Sequence
  Synthetic
gttgatctgtagcagcagcu

SEQ ID NO: 1076 - DNA, 20 bp, Artificial Sequence
  Synthetic
gttgatctgtagcagcagcu

SEQ ID NO: 1077 - DNA, 20 bp, Artificial Sequence
  Synthetic
gttgatctgtagcagcagcu

SEQ ID NO: 1078 - DNA, 20 bp, Artificial Sequence
  Synthetic
gttgatctgtagcagcagcu

SEQ ID NO: 1079 - DNA, 20 bp, Artificial Sequence
  Synthetic
gttgatctgtagcagcagcu

SEQ ID NO: 1080 - DNA, 20 bp, Artificial Sequence
  Synthetic
gttgatctgtagcagcagcu

SEQ ID NO: 1081 - DNA, 20 bp, Artificial Sequence
  Synthetic
gttgatctgtagcagcagcu

SEQ ID NO: 1082 - DNA, 20 bp, Artificial Sequence
  Synthetic
gttgatctgtagcagcagcu

SEQ ID NO: 1083 - DNA, 20 bp, Artificial Sequence
  Synthetic
gttgatctgtagcagcagcu

SEQ ID NO: 1084 - DNA, 20 bp, Artificial Sequence
  Synthetic
gttgatctgtagcagcagcu

SEQ ID NO: 1085 - DNA, 20 bp, Artificial Sequence
  Synthetic
gttgatctgtagcagcagcu

SEQ ID NO: 1086 - DNA, 20 bp, Artificial Sequence
  Synthetic
gttgatctgtagcagcagcu

SEQ ID NO: 1087 - DNA, 20 bp, Artificial Sequence
  Synthetic
gttgatctgtagcagcagcu

SEQ ID NO: 1088 - DNA, 20 bp, Artificial Sequence
  Synthetic
gttgatctgtagcagcagcu

SEQ ID NO: 1089 - DNA, 20 bp, Artificial Sequence
  Synthetic
gttgatctgtagcagcagcu

SEQ ID NO: 1090 - DNA, 20 bp, Artificial Sequence
  Synthetic
gttgatctgtagcagcagcu

SEQ ID NO: 1091 - DNA, 20 bp, Artificial Sequence
  Synthetic
uugatctgtagcagcagctt

SEQ ID NO: 1092 - DNA, 20 bp, Artificial Sequence
  Synthetic
uugauctgtagcagcagctt

SEQ ID NO: 1093 - DNA, 20 bp, Artificial Sequence
  Synthetic
uugatctgtagcagcagctt

SEQ ID NO: 1094 - DNA, 20 bp, Artificial Sequence
  Synthetic
uugatctgtagcagcagctt

SEQ ID NO: 1095 - DNA, 20 bp, Artificial Sequence
  Synthetic
uugatctgtagcagcagctt

SEQ ID NO: 1096 - DNA, 20 bp, Artificial Sequence
  Synthetic
uugauctgtagcagcagctt

SEQ ID NO: 1097 - DNA, 20 bp, Artificial Sequence
  Synthetic
uugauctgtagcagcagctt

SEQ ID NO: 1098 - DNA, 20 bp, Artificial Sequence
  Synthetic
uugatctgtagcagcagctt

SEQ ID NO: 1099 - DNA, 20 bp, Artificial Sequence
  Synthetic
uugatctgtagcagcagctt

SEQ ID NO: 1100 - DNA, 20 bp, Artificial Sequence
  Synthetic
uugauctgtagcagcagctt

SEQ ID NO: 1101 - DNA, 20 bp, Artificial Sequence
  Synthetic
uugatctgtagcagcagctt

SEQ ID NO: 1102 - DNA, 20 bp, Artificial Sequence
  Synthetic
uugatctgtagcagcagctt

SEQ ID NO: 1103 - DNA, 20 bp, Artificial Sequence
  Synthetic
uugatctgtagcagcagctt

SEQ ID NO: 1104 - DNA, 20 bp, Artificial Sequence
  Synthetic
uugauctgtagcagcagctt

SEQ ID NO: 1105 - DNA, 20 bp, Artificial Sequence
  Synthetic
uugauctgtagcagcagctt

SEQ ID NO: 1106 - DNA, 20 bp, Artificial Sequence
  Synthetic
uugatctgtagcagcagctt

SEQ ID NO: 1107 - RNA, 20 nt, Artificial Sequence
  Synthetic
ggcacaagggcacagacuuc

SEQ ID NO: 1108 - RNA, 20 nt, Artificial Sequence
  Synthetic
gcacaagggcacagacuucc

SEQ ID NO: 1109 - RNA, 20 nt, Artificial Sequence
  Synthetic
ggcacaagggcacagacuuc

SEQ ID NO: 1110 - RNA, 20 nt, Artificial Sequence
  Synthetic
gcacaagggcacagacuucc

SEQ ID NO: 1111 - RNA, 20 nt, Artificial Sequence
  Synthetic
ggcacaagggcacagacuuc

SEQ ID NO: 1112 - RNA, 20 nt, Artificial Sequence
  Synthetic
ggcacaagggcacagacuuc

SEQ ID NO: 1113 - RNA, 20 nt, Artificial Sequence
  Synthetic
gcacaagggcacagacuucc

SEQ ID NO: 1114 - RNA, 20 nt, Artificial Sequence
  Synthetic
ggcacaagggcacagacuuc

SEQ ID NO: 1115 - RNA, 20 nt, Artificial Sequence
  Synthetic
ggcacaagggcacagacuuc

SEQ ID NO: 1116 - RNA, 20 nt, Artificial Sequence
  Synthetic
gcacaagggcacagacuucc

SEQ ID NO: 1117 - RNA, 20 nt, Artificial Sequence
  Synthetic
ggcacaagggcacagacuuc

SEQ ID NO: 1118 - RNA, 20 nt, Artificial Sequence
  Synthetic
ggcacaagggcacagacuuc

SEQ ID NO: 1119 - DNA, 20 bp, Artificial Sequence
  Synthetic
gacctctgtgaaagccaaca

SEQ ID NO: 1120 - RNA, 17 nt, Artificial Sequence
  Synthetic
acaagggcacagacuuc

SEQ ID NO: 1121 - RNA, 19 nt, Artificial Sequence
  Synthetic
ggcacaagggcacagacuu

SEQ ID NO: 1122 - RNA, 18 nt, Artificial Sequence
  Synthetic
ggcacaagggcacagacu

SEQ ID NO: 1123 - DNA, 17 bp, Artificial Sequence
  Synthetic
ggcacaagggcacagac

SEQ ID NO: 1124 - DNA, 17 bp, Artificial Sequence
  Synthetic
cacacagtagatgaggg

SEQ ID NO: 1125 - DNA, 19 bp, Artificial Sequence
  Synthetic
gugcacacagtagatgagg

SEQ ID NO: 1126 - DNA, 18 bp, Artificial Sequence
  Synthetic
gugcacacagtagatgag

SEQ ID NO: 1127 - DNA, 17 bp, Artificial Sequence
  Synthetic
gugcacacagtagatga

SEQ ID NO: 1128 - RNA, 20 nt, Artificial Sequence
  Synthetic
ggcacaagggcacagacuuc

SEQ ID NO: 1129 - DNA, 20 bp, Artificial Sequence
  Synthetic
accgccatccccgccguagc

SEQ ID NO: 1130 - DNA, 20 bp, Artificial Sequence
  Synthetic
gttaccgccatccccgccgu

SEQ ID NO: 1131 - DNA, 20 bp, Artificial Sequence
  Synthetic
gttaccgccatccccgccgu

SEQ ID NO: 1132 - DNA, 20 bp, Artificial Sequence
  Synthetic
accgccatccccgccguagc

SEQ ID NO: 1133 - DNA, 20 bp, Artificial Sequence
  Synthetic
accgcgaccctctggacagg

SEQ ID NO: 1134 - DNA, 20 bp, Artificial Sequence
  Synthetic
accgcgaccctctggacagg

SEQ ID NO: 1135 - DNA, 20 bp, Artificial Sequence
  Synthetic
accgcgaccctctggacagg

SEQ ID NO: 1136 - DNA, 20 bp, Artificial Sequence
  Synthetic
accgcgaccctctggacagg

SEQ ID NO: 1137 - DNA, 20 bp, Artificial Sequence
  Synthetic
accgcgaccctctggacagg

SEQ ID NO: 1138 - DNA, 20 bp, Artificial Sequence
  Synthetic
accgcgaccctctggacagg

SEQ ID NO: 1139 - DNA, 20 bp, Artificial Sequence
  Synthetic
accgcgaccctctggacagg

SEQ ID NO: 1140 - DNA, 20 bp, Artificial Sequence
  Synthetic
accgcgaccctctggacagg

SEQ ID NO: 1141 - DNA, 20 bp, Artificial Sequence
  Synthetic
cagcacgcaggccaggggcg

SEQ ID NO: 1142 - DNA, 20 bp, Artificial Sequence
  Synthetic
cagcacgcaggccaggggcg

SEQ ID NO: 1143 - DNA, 20 bp, Artificial Sequence
  Synthetic
cagcacgcaggccaggggcg

SEQ ID NO: 1144 - DNA, 20 bp, Artificial Sequence
  Synthetic
cagcacgcaggccaggggcg

SEQ ID NO: 1145 - DNA, 20 bp, Artificial Sequence
  Synthetic
cagcacgcaggccaggggcg

SEQ ID NO: 1146 - DNA, 20 bp, Artificial Sequence
  Synthetic
cagcacgcaggccaggggcg

SEQ ID NO: 1147 - DNA, 20 bp, Artificial Sequence
  Synthetic
cagcacgcaggccaggggcg

SEQ ID NO: 1148 - DNA, 20 bp, Artificial Sequence
  Synthetic
cagcacgcaggccaggggcg

SEQ ID NO: 1149 - RNA, 20 nt, Artificial Sequence
  Synthetic
ugcccagcacgcaggccagg

SEQ ID NO: 1150 - RNA, 20 nt, Artificial Sequence
  Synthetic
ugcccagcacgcaggccagg

SEQ ID NO: 1151 - RNA, 20 nt, Artificial Sequence
  Synthetic
ugcccagcacgcaggccagg

SEQ ID NO: 1152 - RNA, 20 nt, Artificial Sequence
  Synthetic
ugcccagcacgcaggccagg

SEQ ID NO: 1153 - RNA, 20 nt, Artificial Sequence
  Synthetic
ugcccagcacgcaggccagg

SEQ ID NO: 1154 - RNA, 20 nt, Artificial Sequence
  Synthetic
ugcccagcacgcaggccagg

SEQ ID NO: 1155 - RNA, 20 nt, Artificial Sequence
  Synthetic
ugcccagcacgcaggccagg

SEQ ID NO: 1156 - RNA, 20 nt, Artificial Sequence
  Synthetic
ugcccagcacgcaggccagg

SEQ ID NO: 1157 - DNA, 20 bp, Artificial Sequence
  Synthetic
guugauctgtagcagcagct

SEQ ID NO: 1158 - DNA, 20 bp, Artificial Sequence
  Synthetic
guugauctgtagcagcagct

SEQ ID NO: 1159 - DNA, 20 bp, Artificial Sequence
  Synthetic
guugatctgtagcagcagct

SEQ ID NO: 1160 - DNA, 20 bp, Artificial Sequence
  Synthetic
guugatctgtagcagcagct

SEQ ID NO: 1161 - DNA, 20 bp, Artificial Sequence
  Synthetic
guugauctgtagcagcagct

SEQ ID NO: 1162 - DNA, 20 bp, Artificial Sequence
  Synthetic
guugauctgtagcagcagct

SEQ ID NO: 1163 - DNA, 20 bp, Artificial Sequence
  Synthetic
guugauctgtagcagcagct

SEQ ID NO: 1164 - DNA, 20 bp, Artificial Sequence
  Synthetic
guugauctgtagcagcagct

SEQ ID NO: 1165 - DNA, 20 bp, Artificial Sequence
  Synthetic
guugauctgtagcagcagct

SEQ ID NO: 1166 - DNA, 20 bp, Artificial Sequence
  Synthetic
guugatctgtagcagcagct

SEQ ID NO: 1167 - DNA, 20 bp, Artificial Sequence
  Synthetic
guugatctgtagcagcagct

SEQ ID NO: 1168 - DNA, 20 bp, Artificial Sequence
  Synthetic
guugauctgtagcagcagct

SEQ ID NO: 1169 - DNA, 20 bp, Artificial Sequence
  Synthetic
guugauctgtagcagcagct

SEQ ID NO: 1170 - DNA, 20 bp, Artificial Sequence
  Synthetic
guugauctgtagcagcagct

SEQ ID NO: 1171 - DNA, 20 bp, Artificial Sequence
  Synthetic
guugauctgtagcagcagct

SEQ ID NO: 1172 - DNA, 20 bp, Artificial Sequence
  Synthetic
guugauctgtagcagcagct

SEQ ID NO: 1173 - DNA, 20 bp, Artificial Sequence
  Synthetic
guugauctgtagcagcagct

SEQ ID NO: 1174 - DNA, 20 bp, Artificial Sequence
  Synthetic
guugatctgtagcagcagct

SEQ ID NO: 1175 - DNA, 20 bp, Artificial Sequence
  Synthetic
guugatctgtagcagcagct

SEQ ID NO: 1176 - DNA, 20 bp, Artificial Sequence
  Synthetic
guugatctgtagcagcagct

SEQ ID NO: 1177 - DNA, 20 bp, Artificial Sequence
  Synthetic
guugauctgtagcagcagct

SEQ ID NO: 1178 - DNA, 20 bp, Artificial Sequence
  Synthetic
guugauctgtagcagcagct

SEQ ID NO: 1179 - DNA, 20 bp, Artificial Sequence
  Synthetic
guugauctgtagcagcagct

SEQ ID NO: 1180 - DNA, 20 bp, Artificial Sequence
  Synthetic
guugauctgtagcagcagct

SEQ ID NO: 1181 - DNA, 20 bp, Artificial Sequence
  Synthetic
guugauctgtagcagcagct

SEQ ID NO: 1182 - DNA, 20 bp, Artificial Sequence
  Synthetic
guugauctgtagcagcagct

SEQ ID NO: 1183 - DNA, 20 bp, Artificial Sequence
  Synthetic
guugatttgtagcagcagct

SEQ ID NO: 1184 - DNA, 20 bp, Artificial Sequence
  Synthetic
guugatctgtagtagcagct

SEQ ID NO: 1185 - DNA, 20 bp, Artificial Sequence
  Synthetic
guugauttgtagcagcagct

SEQ ID NO: 1186 - DNA, 20 bp, Artificial Sequence
  Synthetic
guugauctgtagtagcagct

SEQ ID NO: 1187 - DNA, 20 bp, Artificial Sequence
  Synthetic
guugauutgtagcagcagct

SEQ ID NO: 1188 - DNA, 20 bp, Artificial Sequence
  Synthetic
guugauctgtagtagcagct

SEQ ID NO: 1189 - DNA, 20 bp, Artificial Sequence
  Synthetic
guugatttgtagcagcagct

SEQ ID NO: 1190 - DNA, 20 bp, Artificial Sequence
  Synthetic
guugatctgtagtagcagct

SEQ ID NO: 1191 - DNA, 20 bp, Artificial Sequence
  Synthetic
guugatttgtagcagcagct

SEQ ID NO: 1192 - DNA, 20 bp, Artificial Sequence
  Synthetic
guugatctgtagtagcagct

SEQ ID NO: 1193 - DNA, 20 bp, Artificial Sequence
  Synthetic
guugauutgtagcagcagct

SEQ ID NO: 1194 - DNA, 20 bp, Artificial Sequence
  Synthetic
guugauctgtagtagcagct

SEQ ID NO: 1195 - DNA, 20 bp, Artificial Sequence
  Synthetic
guugauttgtagcagcagct

SEQ ID NO: 1196 - DNA, 20 bp, Artificial Sequence
  Synthetic
guugauctgtagtagcagct

SEQ ID NO: 1197 - DNA, 20 bp, Artificial Sequence
  Synthetic
guugauutgtagcagcagct

SEQ ID NO: 1198 - DNA, 20 bp, Artificial Sequence
  Synthetic
guugauctgtagtagcagct

SEQ ID NO: 1199 - DNA, 20 bp, Artificial Sequence
  Synthetic
guugatttgtagcagcagct

SEQ ID NO: 1200 - DNA, 20 bp, Artificial Sequence
  Synthetic
guugatctgtagtagcagct

SEQ ID NO: 1201 - DNA, 20 bp, Artificial Sequence
  Synthetic
guugauttgtagcagcagct

SEQ ID NO: 1202 - DNA, 20 bp, Artificial Sequence
  Synthetic
guugauctgtagtagcagct

SEQ ID NO: 1203 - DNA, 20 bp, Artificial Sequence
  Synthetic
guugauutgtagcagcagct

SEQ ID NO: 1204 - DNA, 20 bp, Artificial Sequence
  Synthetic
guugauctgtagtagcagct

SEQ ID NO: 1205 - DNA, 20 bp, Artificial Sequence
  Synthetic
guugatttgtagcagcagct

SEQ ID NO: 1206 - DNA, 20 bp, Artificial Sequence
  Synthetic
guugatctgtagtagcagct

SEQ ID NO: 1207 - DNA, 20 bp, Artificial Sequence
  Synthetic
guugatttgtagcagcagct

SEQ ID NO: 1208 - DNA, 20 bp, Artificial Sequence
  Synthetic
guugatctgtagtagcagct

SEQ ID NO: 1209 - DNA, 20 bp, Artificial Sequence
  Synthetic
guugauutgtagcagcagct

SEQ ID NO: 1210 - DNA, 20 bp, Artificial Sequence
  Synthetic
guugauctgtagtagcagct

SEQ ID NO: 1211 - DNA, 20 bp, Artificial Sequence
  Synthetic
guugauttgtagcagcagct

SEQ ID NO: 1212 - DNA, 20 bp, Artificial Sequence
  Synthetic
guugauctgtagtagcagct

SEQ ID NO: 1213 - DNA, 20 bp, Artificial Sequence
  Synthetic
guugauutgtagcagcagct

SEQ ID NO: 1214 - DNA, 20 bp, Artificial Sequence
  Synthetic
guugauctgtagtagcagct

SEQ ID NO: 1215 - DNA, 20 bp, Artificial Sequence
  Synthetic
guugatttgtagcagcagct

SEQ ID NO: 1216 - DNA, 20 bp, Artificial Sequence
  Synthetic
guugatctgtagtagcagct

SEQ ID NO: 1217 - DNA, 20 bp, Artificial Sequence
  Synthetic
guugauttgtagcagcagct

SEQ ID NO: 1218 - DNA, 20 bp, Artificial Sequence
  Synthetic
guugauctgtagtagcagct

SEQ ID NO: 1219 - DNA, 20 bp, Artificial Sequence
  Synthetic
guugauutgtagcagcagct

SEQ ID NO: 1220 - DNA, 20 bp, Artificial Sequence
  Synthetic
guugauctgtagtagcagct

SEQ ID NO: 1221 - DNA, 20 bp, Artificial Sequence
  Synthetic
guugauutgtagcagcagct

SEQ ID NO: 1222 - DNA, 20 bp, Artificial Sequence
  Synthetic
guugauctgtagtagcagct

SEQ ID NO: 1223 - DNA, 20 bp, Artificial Sequence
  Synthetic
guugatttgtagcagcagct

SEQ ID NO: 1224 - DNA, 20 bp, Artificial Sequence
  Synthetic
guugatctgtagtagcagct

SEQ ID NO: 1225 - DNA, 20 bp, Artificial Sequence
  Synthetic
guugatttgtagcagcagct

SEQ ID NO: 1226 - DNA, 20 bp, Artificial Sequence
  Synthetic
guugatctgtagtagcagct

SEQ ID NO: 1227 - DNA, 20 bp, Artificial Sequence
  Synthetic
guugatttgtagcagcagct

SEQ ID NO: 1228 - DNA, 20 bp, Artificial Sequence
  Synthetic
guugatctgtagtagcagct

SEQ ID NO: 1229 - DNA, 20 bp, Artificial Sequence
  Synthetic
guugauutgtagcagcagct

SEQ ID NO: 1230 - DNA, 20 bp, Artificial Sequence
  Synthetic
guugauctgtagtagcagct

SEQ ID NO: 1231 - DNA, 20 bp, Artificial Sequence
  Synthetic
guugauutgtagcagcagct

SEQ ID NO: 1232 - DNA, 20 bp, Artificial Sequence
  Synthetic
guugauctgtagtagcagct

SEQ ID NO: 1233 - DNA, 20 bp, Artificial Sequence
  Synthetic
guugauttgtagcagcagct

SEQ ID NO: 1234 - DNA, 20 bp, Artificial Sequence
  Synthetic
guugauctgtagtagcagct

SEQ ID NO: 1235 - DNA, 20 bp, Artificial Sequence
  Synthetic
guugauttgtagcagcagct

SEQ ID NO: 1236 - DNA, 20 bp, Artificial Sequence
  Synthetic
guugauctgtagtagcagct

SEQ ID NO: 1237 - DNA, 20 bp, Artificial Sequence
  Synthetic
guugauutgtagcagcagct

SEQ ID NO: 1238 - DNA, 20 bp, Artificial Sequence
  Synthetic
guugauctgtagtagcagct

SEQ ID NO: 1239 - DNA, 20 bp, Artificial Sequence
  Synthetic
guugauutgtagcagcagct

SEQ ID NO: 1240 - DNA, 20 bp, Artificial Sequence
  Synthetic
guugauctgtagtagcagct

SEQ ID NO: 1241 - DNA, 20 bp, Artificial Sequence
  Synthetic
guugatctgtagcagcagct

SEQ ID NO: 1242 - DNA, 20 bp, Artificial Sequence
  Synthetic
guugatctgtagcagcagct

SEQ ID NO: 1243 - DNA, 20 bp, Artificial Sequence
  Synthetic
guugatctgtagcagcagct

SEQ ID NO: 1244 - DNA, 20 bp, Artificial Sequence
  Synthetic
guugatctgtagcagcagct

SEQ ID NO: 1245 - DNA, 20 bp, Artificial Sequence
  Synthetic
guugatctgtagtagcagct

SEQ ID NO: 1246 - DNA, 20 bp, Artificial Sequence
  Synthetic
guugatctgtagcagcagct

SEQ ID NO: 1247 - DNA, 20 bp, Artificial Sequence
  Synthetic
guugatctgtagcagcagct

SEQ ID NO: 1248 - DNA, 20 bp, Artificial Sequence
  Synthetic
guugatctgtagcagcagct

SEQ ID NO: 1249 - DNA, 20 bp, Artificial Sequence
  Synthetic
guugatctgtagcagcagct

SEQ ID NO: 1250 - DNA, 20 bp, Artificial Sequence
  Synthetic
guugatctgtagcagcagct

SEQ ID NO: 1251 - DNA, 20 bp, Artificial Sequence
  Synthetic
guugatctgtagtagcagct

SEQ ID NO: 1252 - DNA, 20 bp, Artificial Sequence
  Synthetic
guugatctgtagcagcagct

SEQ ID NO: 1253 - DNA, 20 bp, Artificial Sequence
  Synthetic
guugatctgtagcagcagct

SEQ ID NO: 1254 - DNA, 20 bp, Artificial Sequence
  Synthetic
guugatctgtagcagcagct

SEQ ID NO: 1255 - DNA, 20 bp, Artificial Sequence
  Synthetic
guugatctgtagcagcagct

SEQ ID NO: 1256 - DNA, 20 bp, Artificial Sequence
  Synthetic
guugatctgtagcagcagct

SEQ ID NO: 1257 - DNA, 20 bp, Artificial Sequence
  Synthetic
guugatctgtagtagcagct

SEQ ID NO: 1258 - DNA, 20 bp, Artificial Sequence
  Synthetic
guugatctgtagcagcagct

SEQ ID NO: 1259 - DNA, 20 bp, Artificial Sequence
  Synthetic
guugatctgtagcagcagct

SEQ ID NO: 1260 - DNA, 20 bp, Artificial Sequence
  Synthetic
guugatctgtagcagcagct

SEQ ID NO: 1261 - DNA, 20 bp, Artificial Sequence
  Synthetic
guugatctgtagtagcagct

SEQ ID NO: 1262 - DNA, 20 bp, Artificial Sequence
  Synthetic
guugatctgtagcagcagct

SEQ ID NO: 1263 - DNA, 20 bp, Artificial Sequence
  Synthetic
guugatctgtagcagcagct

SEQ ID NO: 1264 - DNA, 20 bp, Artificial Sequence
  Synthetic
guugatctgtagcagcagct

SEQ ID NO: 1265 - DNA, 20 bp, Artificial Sequence
  Synthetic
guugatctgtagcagcagct

SEQ ID NO: 1266 - DNA, 20 bp, Artificial Sequence
  Synthetic
guugatctgtagcagcagct

SEQ ID NO: 1267 - DNA, 20 bp, Artificial Sequence
  Synthetic
guugatctgtagcagcagct

SEQ ID NO: 1268 - DNA, 20 bp, Artificial Sequence
  Synthetic
guugatctgtagcagcagct

SEQ ID NO: 1269 - DNA, 20 bp, Artificial Sequence
  Synthetic
guugatctgtagcagcagct

SEQ ID NO: 1270 - DNA, 20 bp, Artificial Sequence
  Synthetic
guugatctgtagcagcagct

SEQ ID NO: 1271 - DNA, 20 bp, Artificial Sequence
  Synthetic
guugatctgtagcagcagct

SEQ ID NO: 1272 - DNA, 20 bp, Artificial Sequence
  Synthetic
guugatctgtagcagcagct

SEQ ID NO: 1273 - DNA, 20 bp, Artificial Sequence
  Synthetic
guugatctgtagcagcagct

SEQ ID NO: 1274 - DNA, 20 bp, Artificial Sequence
  Synthetic
ttgatctgtagcagcagcuu

SEQ ID NO: 1275 - DNA, 20 bp, Artificial Sequence
  Synthetic
gttgatctgtagcagcagcu

SEQ ID NO: 1276 - DNA, 20 bp, Artificial Sequence
  Synthetic
ggttgatctgtagcagcagc

SEQ ID NO: 1277 - DNA, 20 bp, Artificial Sequence
  Synthetic
guugatctgtagcagcagct

SEQ ID NO: 1278 - DNA, 20 bp, Artificial Sequence
  Synthetic
gttgatctgtagcagcagcu

SEQ ID NO: 1279 - DNA, 20 bp, Artificial Sequence
  Synthetic
cacaagggcgcagacttcca

SEQ ID NO: 1280 - DNA, 20 bp, Artificial Sequence
  Synthetic
guugatctgtagcagcagct

SEQ ID NO: 1281 - DNA, 20 bp, Artificial Sequence
  Synthetic
guugatctgtagtagcagct

SEQ ID NO: 1282 - DNA, 20 bp, Artificial Sequence
  Synthetic
guugatctgtagcagcagct

SEQ ID NO: 1283 - DNA, 20 bp, Artificial Sequence
  Synthetic
guugatctgtagtagcagct

SEQ ID NO: 1284 - RNA, 20 nt, Artificial Sequence
  Synthetic
ggcacaagggcacagacuuc

SEQ ID NO: 1285 - RNA, 20 nt, Artificial Sequence
  Synthetic
ggcacaagggcacagacuuc

SEQ ID NO: 1286 - RNA, 20 nt, Artificial Sequence
  Synthetic
ggcacaagggcacagacuuc

SEQ ID NO: 1287 - RNA, 20 nt, Artificial Sequence
  Synthetic
ggcacaagggcacagacuuc

SEQ ID NO: 1288 - RNA, 20 nt, Artificial Sequence
  Synthetic
ggcacaagggcacagacuuc

SEQ ID NO: 1289 - RNA, 20 nt, Artificial Sequence
  Synthetic
ggcacaagggcacagacuuc

SEQ ID NO: 1290 - RNA, 20 nt, Artificial Sequence
  Synthetic
ggcacaagggcacagacuuc

SEQ ID NO: 1291 - RNA, 20 nt, Artificial Sequence
  Synthetic
ggcacaagggcacagacuuc

SEQ ID NO: 1292 - DNA, 20 bp, Artificial Sequence
  Synthetic
gugcacacagtagatgaggg

SEQ ID NO: 1293 - DNA, 20 bp, Artificial Sequence
  Synthetic
gugcacacagtagatgaggg

SEQ ID NO: 1294 - DNA, 20 bp, Artificial Sequence
  Synthetic
gugcacacagtagatgaggg

SEQ ID NO: 1295 - DNA, 20 bp, Artificial Sequence
  Synthetic
gugcacacagtagatgaggg

SEQ ID NO: 1296 - DNA, 20 bp, Artificial Sequence
  Synthetic
gugcacacagtagatgaggg

SEQ ID NO: 1297 - DNA, 20 bp, Artificial Sequence
  Synthetic
gugcacacagtagatgaggg

SEQ ID NO: 1298 - DNA, 20 bp, Artificial Sequence
  Synthetic
gugcacacagtagatgaggg

SEQ ID NO: 1299 - DNA, 20 bp, Artificial Sequence
  Synthetic
gugcacacagtagatgaggg

SEQ ID NO: 1300 - DNA, 20 bp, Artificial Sequence
  Synthetic
gugcacacagtagatgaggg

SEQ ID NO: 1301 - RNA, 20 nt, Artificial Sequence
  Synthetic
ggcacaagggcacagacuuc

SEQ ID NO: 1302 - RNA, 21 nt, Artificial Sequence
  Synthetic
ggcacaaagggcacagacuuc

SEQ ID NO: 1303 - DNA, 20 bp, Artificial Sequence
  Synthetic
gugcacacagtagatgaggg

SEQ ID NO: 1304 - DNA, 21 bp, Artificial Sequence
  Synthetic
gugcaacacagtagatgaggg

SEQ ID NO: 1305 - RNA, 15 nt, Artificial Sequence
  Synthetic
aagggcacagacuuc

SEQ ID NO: 1306 - RNA, 14 nt, Artificial Sequence
  Synthetic
agggcacagacuuc

SEQ ID NO: 1307 - RNA, 13 nt, Artificial Sequence
  Synthetic
gggcacagacuuc

SEQ ID NO: 1308 - RNA, 12 nt, Artificial Sequence
  Synthetic
ggcacagacuuc

SEQ ID NO: 1309 - RNA, 11 nt, Artificial Sequence
  Synthetic
gcacagacuuc

SEQ ID NO: 1310 - RNA, 10 nt, Artificial Sequence
  Synthetic
cacagacuuc

SEQ ID NO: 1311 - RNA, 9 nt, Artificial Sequence
  Synthetic
acagacuuc

SEQ ID NO: 1312 - RNA, 8 nt, Artificial Sequence
  Synthetic
cagacuuc

SEQ ID NO: 1313 - RNA, 7 nt, Artificial Sequence
  Synthetic
agacuuc

SEQ ID NO: 1314 - RNA, 6 nt, Artificial Sequence
  Synthetic
gacuuc

SEQ ID NO: 1315 - RNA, 16 nt, Artificial Sequence
  Synthetic
caagggcacagacuuc

SEQ ID NO: 1316 - RNA, 15 nt, Artificial Sequence
  Synthetic
aagggcacagacuuc

SEQ ID NO: 1317 - RNA, 14 nt, Artificial Sequence
  Synthetic
agggcacagacuuc

SEQ ID NO: 1318 - RNA, 13 nt, Artificial Sequence
  Synthetic
gggcacagacuuc

SEQ ID NO: 1319 - RNA, 12 nt, Artificial Sequence
  Synthetic
ggcacagacuuc

SEQ ID NO: 1320 - RNA, 11 nt, Artificial Sequence
  Synthetic
gcacagacuuc

SEQ ID NO: 1321 - RNA, 10 nt, Artificial Sequence
  Synthetic
cacagacuuc

SEQ ID NO: 1322 - RNA, 9 nt, Artificial Sequence
  Synthetic
acagacuuc

SEQ ID NO: 1323 - RNA, 8 nt, Artificial Sequence
  Synthetic
cagacuuc

SEQ ID NO: 1324 - RNA, 7 nt, Artificial Sequence
  Synthetic
agacuuc

SEQ ID NO: 1325 - RNA, 6 nt, Artificial Sequence
  Synthetic
gacuuc

SEQ ID NO: 1326 - DNA, 20 bp, Artificial Sequence
  Synthetic
tctccattctatcttauguu

SEQ ID NO: 1327 - DNA, 20 bp, Artificial Sequence
  Synthetic
cacagtagatgagggagcag

SEQ ID NO: 1328 - DNA, 20 bp, Artificial Sequence
  Synthetic
cacagtagatgagggagcag

SEQ ID NO: 1329 - DNA, 20 bp, Artificial Sequence
  Synthetic
cacagtagatgagggagcag

SEQ ID NO: 1330 - DNA, 20 bp, Artificial Sequence
  Synthetic
acacagtagatgagggagca

SEQ ID NO: 1331 - DNA, 20 bp, Artificial Sequence
  Synthetic
acacagtagatgagggagca

SEQ ID NO: 1332 - DNA, 20 bp, Artificial Sequence
  Synthetic
acacagtagatgagggagca

SEQ ID NO: 1333 - DNA, 20 bp, Artificial Sequence
  Synthetic
cacacagtagatgagggagc

SEQ ID NO: 1334 - DNA, 20 bp, Artificial Sequence
  Synthetic
cacacagtagatgagggagc

SEQ ID NO: 1335 - DNA, 20 bp, Artificial Sequence
  Synthetic
cacacagtagatgagggagc

SEQ ID NO: 1336 - DNA, 20 bp, Artificial Sequence
  Synthetic
gcacacagtagatgagggag

SEQ ID NO: 1337 - DNA, 20 bp, Artificial Sequence
  Synthetic
gcacacagtagatgagggag

SEQ ID NO: 1338 - DNA, 20 bp, Artificial Sequence
  Synthetic
gcacacagtagatgagggag

SEQ ID NO: 1339 - DNA, 20 bp, Artificial Sequence
  Synthetic
tgcacacagtagatgaggga

SEQ ID NO: 1340 - DNA, 20 bp, Artificial Sequence
  Synthetic
tgcacacagtagatgaggga

SEQ ID NO: 1341 - DNA, 20 bp, Artificial Sequence
  Synthetic
tgcacacagtagatgaggga

SEQ ID NO: 1342 - DNA, 20 bp, Artificial Sequence
  Synthetic
gtgcacacagtagatgaggg

SEQ ID NO: 1343 - DNA, 20 bp, Artificial Sequence
  Synthetic
gtgcacacagtagatgaggg

SEQ ID NO: 1344 - DNA, 20 bp, Artificial Sequence
  Synthetic
gtgcacacagtagatgaggg

SEQ ID NO: 1345 - DNA, 20 bp, Artificial Sequence
  Synthetic
cacagtagatgagggagcag

SEQ ID NO: 1346 - DNA, 20 bp, Artificial Sequence
  Synthetic
cacagtagatgagggagcag

SEQ ID NO: 1347 - DNA, 20 bp, Artificial Sequence
  Synthetic
cacagtagatgagggagcag

SEQ ID NO: 1348 - DNA, 20 bp, Artificial Sequence
  Synthetic
acacagtagatgagggagca

SEQ ID NO: 1349 - DNA, 20 bp, Artificial Sequence
  Synthetic
acacagtagatgagggagca

SEQ ID NO: 1350 - DNA, 20 bp, Artificial Sequence
  Synthetic
acacagtagatgagggagca

SEQ ID NO: 1351 - DNA, 20 bp, Artificial Sequence
  Synthetic
cacacagtagatgagggagc

SEQ ID NO: 1352 - DNA, 20 bp, Artificial Sequence
  Synthetic
cacacagtagatgagggagc

SEQ ID NO: 1353 - DNA, 20 bp, Artificial Sequence
  Synthetic
cacacagtagatgagggagc

SEQ ID NO: 1354 - DNA, 20 bp, Artificial Sequence
  Synthetic
gcacacagtagatgagggag

SEQ ID NO: 1355 - DNA, 20 bp, Artificial Sequence
  Synthetic
gcacacagtagatgagggag

SEQ ID NO: 1356 - DNA, 20 bp, Artificial Sequence
  Synthetic
gcacacagtagatgagggag

SEQ ID NO: 1357 - DNA, 20 bp, Artificial Sequence
  Synthetic
tgcacacagtagatgaggga

SEQ ID NO: 1358 - DNA, 20 bp, Artificial Sequence
  Synthetic
tgcacacagtagatgaggga

SEQ ID NO: 1359 - DNA, 20 bp, Artificial Sequence
  Synthetic
tgcacacagtagatgaggga

SEQ ID NO: 1360 - DNA, 20 bp, Artificial Sequence
  Synthetic
gtgcacacagtagatgaggg

SEQ ID NO: 1361 - DNA, 20 bp, Artificial Sequence
  Synthetic
gtgcacacagtagatgaggg

SEQ ID NO: 1362 - DNA, 20 bp, Artificial Sequence
  Synthetic
gtgcacacagtagatgaggg

SEQ ID NO: 1363 - DNA, 20 bp, Artificial Sequence
  Synthetic
cacagtagatgagggagcag

SEQ ID NO: 1364 - DNA, 20 bp, Artificial Sequence
  Synthetic
cacagtagatgagggagcag

SEQ ID NO: 1365 - DNA, 20 bp, Artificial Sequence
  Synthetic
cacagtagatgagggagcag

SEQ ID NO: 1366 - DNA, 20 bp, Artificial Sequence
  Synthetic
acacagtagatgagggagca

SEQ ID NO: 1367 - DNA, 20 bp, Artificial Sequence
  Synthetic
acacagtagatgagggagca

SEQ ID NO: 1368 - DNA, 20 bp, Artificial Sequence
  Synthetic
acacagtagatgagggagca

SEQ ID NO: 1369 - DNA, 20 bp, Artificial Sequence
  Synthetic
cacacagtagatgagggagc

SEQ ID NO: 1370 - DNA, 20 bp, Artificial Sequence
  Synthetic
cacacagtagatgagggagc

SEQ ID NO: 1371 - DNA, 20 bp, Artificial Sequence
  Synthetic
cacacagtagatgagggagc

SEQ ID NO: 1372 - DNA, 20 bp, Artificial Sequence
  Synthetic
gcacacagtagatgagggag

SEQ ID NO: 1373 - DNA, 20 bp, Artificial Sequence
  Synthetic
gcacacagtagatgagggag

SEQ ID NO: 1374 - DNA, 20 bp, Artificial Sequence
  Synthetic
gcacacagtagatgagggag

SEQ ID NO: 1375 - DNA, 20 bp, Artificial Sequence
  Synthetic
ugcacacagtagatgaggga

SEQ ID NO: 1376 - DNA, 20 bp, Artificial Sequence
  Synthetic
ugcacacagtagatgaggga

SEQ ID NO: 1377 - DNA, 20 bp, Artificial Sequence
  Synthetic
ugcacacagtagatgaggga

SEQ ID NO: 1378 - DNA, 20 bp, Artificial Sequence
  Synthetic
gugcacacagtagatgaggg

SEQ ID NO: 1379 - DNA, 20 bp, Artificial Sequence
  Synthetic
gugcacacagtagatgaggg

SEQ ID NO: 1380 - DNA, 20 bp, Artificial Sequence
  Synthetic
gugcacacagtagatgaggg

SEQ ID NO: 1381 - DNA, 20 bp, Artificial Sequence
  Synthetic
cacagtagatgagggagcag

SEQ ID NO: 1382 - DNA, 20 bp, Artificial Sequence
  Synthetic
cacagtagatgagggagcag

SEQ ID NO: 1383 - DNA, 20 bp, Artificial Sequence
  Synthetic
cacagtagatgagggagcag

SEQ ID NO: 1384 - DNA, 20 bp, Artificial Sequence
  Synthetic
acacagtagatgagggagca

SEQ ID NO: 1385 - DNA, 20 bp, Artificial Sequence
  Synthetic
acacagtagatgagggagca

SEQ ID NO: 1386 - DNA, 20 bp, Artificial Sequence
  Synthetic
acacagtagatgagggagca

SEQ ID NO: 1387 - DNA, 20 bp, Artificial Sequence
  Synthetic
cacacagtagatgagggagc

SEQ ID NO: 1388 - DNA, 20 bp, Artificial Sequence
  Synthetic
cacacagtagatgagggagc

SEQ ID NO: 1389 - DNA, 20 bp, Artificial Sequence
  Synthetic
cacacagtagatgagggagc

SEQ ID NO: 1390 - DNA, 20 bp, Artificial Sequence
  Synthetic
gcacacagtagatgagggag

SEQ ID NO: 1391 - DNA, 20 bp, Artificial Sequence
  Synthetic
gcacacagtagatgagggag

SEQ ID NO: 1392 - DNA, 20 bp, Artificial Sequence
  Synthetic
gcacacagtagatgagggag

SEQ ID NO: 1393 - DNA, 20 bp, Artificial Sequence
  Synthetic
tgcacacagtagatgaggga

SEQ ID NO: 1394 - DNA, 20 bp, Artificial Sequence
  Synthetic
tgcacacagtagatgaggga

SEQ ID NO: 1395 - DNA, 20 bp, Artificial Sequence
  Synthetic
tgcacacagtagatgaggga

SEQ ID NO: 1396 - DNA, 20 bp, Artificial Sequence
  Synthetic
gtgcacacagtagatgaggg

SEQ ID NO: 1397 - DNA, 20 bp, Artificial Sequence
  Synthetic
gtgcacacagtagatgaggg

SEQ ID NO: 1398 - DNA, 20 bp, Artificial Sequence
  Synthetic
gtgcacacagtagatgaggg

SEQ ID NO: 1399 - DNA, 20 bp, Artificial Sequence
  Synthetic
cacagtagatgagggagcag

SEQ ID NO: 1400 - DNA, 20 bp, Artificial Sequence
  Synthetic
cacagtagatgagggagcag

SEQ ID NO: 1401 - DNA, 20 bp, Artificial Sequence
  Synthetic
cacagtagatgagggagcag

SEQ ID NO: 1402 - DNA, 20 bp, Artificial Sequence
  Synthetic
acacagtagatgagggagca

SEQ ID NO: 1403 - DNA, 20 bp, Artificial Sequence
  Synthetic
acacagtagatgagggagca

SEQ ID NO: 1404 - DNA, 20 bp, Artificial Sequence
  Synthetic
acacagtagatgagggagca

SEQ ID NO: 1405 - DNA, 20 bp, Artificial Sequence
  Synthetic
cacacagtagatgagggagc

SEQ ID NO: 1406 - DNA, 20 bp, Artificial Sequence
  Synthetic
cacacagtagatgagggagc

SEQ ID NO: 1407 - DNA, 20 bp, Artificial Sequence
  Synthetic
cacacagtagatgagggagc

SEQ ID NO: 1408 - DNA, 20 bp, Artificial Sequence
  Synthetic
gcacacagtagatgagggag

SEQ ID NO: 1409 - DNA, 20 bp, Artificial Sequence
  Synthetic
gcacacagtagatgagggag

SEQ ID NO: 1410 - DNA, 20 bp, Artificial Sequence
  Synthetic
gcacacagtagatgagggag

SEQ ID NO: 1411 - DNA, 20 bp, Artificial Sequence
  Synthetic
tgcacacagtagatgaggga

SEQ ID NO: 1412 - DNA, 20 bp, Artificial Sequence
  Synthetic
tgcacacagtagatgaggga

SEQ ID NO: 1413 - DNA, 20 bp, Artificial Sequence
  Synthetic
tgcacacagtagatgaggga

SEQ ID NO: 1414 - DNA, 20 bp, Artificial Sequence
  Synthetic
gtgcacacagtagatgaggg

SEQ ID NO: 1415 - DNA, 20 bp, Artificial Sequence
  Synthetic
gtgcacacagtagatgaggg

SEQ ID NO: 1416 - DNA, 20 bp, Artificial Sequence
  Synthetic
gtgcacacagtagatgaggg

SEQ ID NO: 1417 - DNA, 20 bp, Artificial Sequence
  Synthetic
cacagtagatgagggagcag

SEQ ID NO: 1418 - DNA, 20 bp, Artificial Sequence
  Synthetic
cacagtagatgagggagcag

SEQ ID NO: 1419 - DNA, 20 bp, Artificial Sequence
  Synthetic
cacagtagatgagggagcag

SEQ ID NO: 1420 - DNA, 20 bp, Artificial Sequence
  Synthetic
acacagtagatgagggagca

SEQ ID NO: 1421 - DNA, 20 bp, Artificial Sequence
  Synthetic
acacagtagatgagggagca

SEQ ID NO: 1422 - DNA, 20 bp, Artificial Sequence
  Synthetic
acacagtagatgagggagca

SEQ ID NO: 1423 - DNA, 20 bp, Artificial Sequence
  Synthetic
cacacagtagatgagggagc

SEQ ID NO: 1424 - DNA, 20 bp, Artificial Sequence
  Synthetic
cacacagtagatgagggagc

SEQ ID NO: 1425 - DNA, 20 bp, Artificial Sequence
  Synthetic
cacacagtagatgagggagc

SEQ ID NO: 1426 - DNA, 20 bp, Artificial Sequence
  Synthetic
gcacacagtagatgagggag

SEQ ID NO: 1427 - DNA, 20 bp, Artificial Sequence
  Synthetic
gcacacagtagatgagggag

SEQ ID NO: 1428 - DNA, 20 bp, Artificial Sequence
  Synthetic
gcacacagtagatgagggag

SEQ ID NO: 1429 - DNA, 20 bp, Artificial Sequence
  Synthetic
tgcacacagtagatgaggga

SEQ ID NO: 1430 - DNA, 20 bp, Artificial Sequence
  Synthetic
tgcacacagtagatgaggga

SEQ ID NO: 1431 - DNA, 20 bp, Artificial Sequence
  Synthetic
tgcacacagtagatgaggga

SEQ ID NO: 1432 - DNA, 20 bp, Artificial Sequence
  Synthetic
gtgcacacagtagatgaggg

SEQ ID NO: 1433 - DNA, 20 bp, Artificial Sequence
  Synthetic
gtgcacacagtagatgaggg

SEQ ID NO: 1434 - DNA, 20 bp, Artificial Sequence
  Synthetic
gtgcacacagtagatgaggg

SEQ ID NO: 1435 - DNA, 20 bp, Artificial Sequence
  Synthetic
cacagtagatgagggagcag

SEQ ID NO: 1436 - DNA, 20 bp, Artificial Sequence
  Synthetic
cacagtagatgagggagcag

SEQ ID NO: 1437 - DNA, 20 bp, Artificial Sequence
  Synthetic
cacagtagatgagggagcag

SEQ ID NO: 1438 - DNA, 20 bp, Artificial Sequence
  Synthetic
acacagtagatgagggagca

SEQ ID NO: 1439 - DNA, 20 bp, Artificial Sequence
  Synthetic
acacagtagatgagggagca

SEQ ID NO: 1440 - DNA, 20 bp, Artificial Sequence
  Synthetic
acacagtagatgagggagca

SEQ ID NO: 1441 - DNA, 20 bp, Artificial Sequence
  Synthetic
cacacagtagatgagggagc

SEQ ID NO: 1442 - DNA, 20 bp, Artificial Sequence
  Synthetic
cacacagtagatgagggagc

SEQ ID NO: 1443 - DNA, 20 bp, Artificial Sequence
  Synthetic
cacacagtagatgagggagc

SEQ ID NO: 1444 - DNA, 20 bp, Artificial Sequence
  Synthetic
gcacacagtagatgagggag

SEQ ID NO: 1445 - DNA, 20 bp, Artificial Sequence
  Synthetic
gcacacagtagatgagggag

SEQ ID NO: 1446 - DNA, 20 bp, Artificial Sequence
  Synthetic
gcacacagtagatgagggag

SEQ ID NO: 1447 - DNA, 20 bp, Artificial Sequence
  Synthetic
ugcacacagtagatgaggga

SEQ ID NO: 1448 - DNA, 20 bp, Artificial Sequence
  Synthetic
ugcacacagtagatgaggga

SEQ ID NO: 1449 - DNA, 20 bp, Artificial Sequence
  Synthetic
ugcacacagtagatgaggga

SEQ ID NO: 1450 - DNA, 20 bp, Artificial Sequence
  Synthetic
gugcacacagtagatgaggg

SEQ ID NO: 1451 - DNA, 20 bp, Artificial Sequence
  Synthetic
gugcacacagtagatgaggg

SEQ ID NO: 1452 - DNA, 20 bp, Artificial Sequence
  Synthetic
gugcacacagtagatgaggg

SEQ ID NO: 1453 - DNA, 20 bp, Artificial Sequence
  Synthetic
cacagtagatgagggagcag

SEQ ID NO: 1454 - DNA, 20 bp, Artificial Sequence
  Synthetic
cacagtagatgagggagcag

SEQ ID NO: 1455 - DNA, 20 bp, Artificial Sequence
  Synthetic
cacagtagatgagggagcag

SEQ ID NO: 1456 - DNA, 20 bp, Artificial Sequence
  Synthetic
acacagtagatgagggagca

SEQ ID NO: 1457 - DNA, 20 bp, Artificial Sequence
  Synthetic
acacagtagatgagggagca

SEQ ID NO: 1458 - DNA, 20 bp, Artificial Sequence
  Synthetic
acacagtagatgagggagca

SEQ ID NO: 1459 - DNA, 20 bp, Artificial Sequence
  Synthetic
cacacagtagatgagggagc

SEQ ID NO: 1460 - DNA, 20 bp, Artificial Sequence
  Synthetic
cacacagtagatgagggagc

SEQ ID NO: 1461 - DNA, 20 bp, Artificial Sequence
  Synthetic
cacacagtagatgagggagc

SEQ ID NO: 1462 - DNA, 20 bp, Artificial Sequence
  Synthetic
gcacacagtagatgagggag

SEQ ID NO: 1463 - DNA, 20 bp, Artificial Sequence
  Synthetic
gcacacagtagatgagggag

SEQ ID NO: 1464 - DNA, 20 bp, Artificial Sequence
  Synthetic
gcacacagtagatgagggag

SEQ ID NO: 1465 - DNA, 20 bp, Artificial Sequence
  Synthetic
tgcacacagtagatgaggga

SEQ ID NO: 1466 - DNA, 20 bp, Artificial Sequence
  Synthetic
tgcacacagtagatgaggga

SEQ ID NO: 1467 - DNA, 20 bp, Artificial Sequence
  Synthetic
tgcacacagtagatgaggga

SEQ ID NO: 1468 - DNA, 20 bp, Artificial Sequence
  Synthetic
gtgcacacagtagatgaggg

SEQ ID NO: 1469 - DNA, 20 bp, Artificial Sequence
  Synthetic
gtgcacacagtagatgaggg

SEQ ID NO: 1470 - DNA, 20 bp, Artificial Sequence
  Synthetic
gtgcacacagtagatgaggg

SEQ ID NO: 1471 - DNA, 20 bp, Artificial Sequence
  Synthetic
cacagtagatgagggagcag

SEQ ID NO: 1472 - DNA, 20 bp, Artificial Sequence
  Synthetic
cacagtagatgagggagcag

SEQ ID NO: 1473 - DNA, 20 bp, Artificial Sequence
  Synthetic
cacagtagatgagggagcag

SEQ ID NO: 1474 - DNA, 20 bp, Artificial Sequence
  Synthetic
acacagtagatgagggagca

SEQ ID NO: 1475 - DNA, 20 bp, Artificial Sequence
  Synthetic
acacagtagatgagggagca

SEQ ID NO: 1476 - DNA, 20 bp, Artificial Sequence
  Synthetic
acacagtagatgagggagca

SEQ ID NO: 1477 - DNA, 20 bp, Artificial Sequence
  Synthetic
cacacagtagatgagggagc

SEQ ID NO: 1478 - DNA, 20 bp, Artificial Sequence
  Synthetic
cacacagtagatgagggagc

SEQ ID NO: 1479 - DNA, 20 bp, Artificial Sequence
  Synthetic
cacacagtagatgagggagc

SEQ ID NO: 1480 - DNA, 20 bp, Artificial Sequence
  Synthetic
gcacacagtagatgagggag

SEQ ID NO: 1481 - DNA, 20 bp, Artificial Sequence
  Synthetic
gcacacagtagatgagggag

SEQ ID NO: 1482 - DNA, 20 bp, Artificial Sequence
  Synthetic
gcacacagtagatgagggag

SEQ ID NO: 1483 - DNA, 20 bp, Artificial Sequence
  Synthetic
tgcacacagtagatgaggga

SEQ ID NO: 1484 - DNA, 20 bp, Artificial Sequence
  Synthetic
tgcacacagtagatgaggga

SEQ ID NO: 1485 - DNA, 20 bp, Artificial Sequence
  Synthetic
tgcacacagtagatgaggga

SEQ ID NO: 1486 - DNA, 20 bp, Artificial Sequence
  Synthetic
gtgcacacagtagatgaggg

SEQ ID NO: 1487 - DNA, 20 bp, Artificial Sequence
  Synthetic
gtgcacacagtagatgaggg

SEQ ID NO: 1488 - DNA, 20 bp, Artificial Sequence
  Synthetic
gtgcacacagtagatgaggg

SEQ ID NO: 1489 - DNA, 20 bp, Artificial Sequence
  Synthetic
cacagtagatgagggagcag

SEQ ID NO: 1490 - DNA, 20 bp, Artificial Sequence
  Synthetic
cacagtagatgagggagcag

SEQ ID NO: 1491 - DNA, 20 bp, Artificial Sequence
  Synthetic
cacagtagatgagggagcag

SEQ ID NO: 1492 - DNA, 20 bp, Artificial Sequence
  Synthetic
acacagtagatgagggagca

SEQ ID NO: 1493 - DNA, 20 bp, Artificial Sequence
  Synthetic
acacagtagatgagggagca

SEQ ID NO: 1494 - DNA, 20 bp, Artificial Sequence
  Synthetic
acacagtagatgagggagca

SEQ ID NO: 1495 - DNA, 20 bp, Artificial Sequence
  Synthetic
cacacagtagatgagggagc

SEQ ID NO: 1496 - DNA, 20 bp, Artificial Sequence
  Synthetic
cacacagtagatgagggagc

SEQ ID NO: 1497 - DNA, 20 bp, Artificial Sequence
  Synthetic
cacacagtagatgagggagc

SEQ ID NO: 1498 - DNA, 20 bp, Artificial Sequence
  Synthetic
gcacacagtagatgagggag

SEQ ID NO: 1499 - DNA, 20 bp, Artificial Sequence
  Synthetic
gcacacagtagatgagggag

SEQ ID NO: 1500 - DNA, 20 bp, Artificial Sequence
  Synthetic
gcacacagtagatgagggag

SEQ ID NO: 1501 - DNA, 20 bp, Artificial Sequence
  Synthetic
ugcacacagtagatgaggga

SEQ ID NO: 1502 - DNA, 20 bp, Artificial Sequence
  Synthetic
ugcacacagtagatgaggga

SEQ ID NO: 1503 - DNA, 20 bp, Artificial Sequence
  Synthetic
ugcacacagtagatgaggga

SEQ ID NO: 1504 - DNA, 20 bp, Artificial Sequence
  Synthetic
gugcacacagtagatgaggg

SEQ ID NO: 1505 - DNA, 20 bp, Artificial Sequence
  Synthetic
gugcacacagtagatgaggg

SEQ ID NO: 1506 - DNA, 20 bp, Artificial Sequence
  Synthetic
gugcacacagtagatgaggg

SEQ ID NO: 1507 - DNA, 20 bp, Artificial Sequence
  Synthetic
cacagtagatgagggagcag

SEQ ID NO: 1508 - DNA, 20 bp, Artificial Sequence
  Synthetic
cacagtagatgagggagcag

SEQ ID NO: 1509 - DNA, 20 bp, Artificial Sequence
  Synthetic
cacagtagatgagggagcag

SEQ ID NO: 1510 - DNA, 20 bp, Artificial Sequence
  Synthetic
cacagtagatgagggagcag

SEQ ID NO: 1511 - DNA, 20 bp, Artificial Sequence
  Synthetic
cacagtagatgagggagcag

SEQ ID NO: 1512 - DNA, 20 bp, Artificial Sequence
  Synthetic
cacagtagatgagggagcag

SEQ ID NO: 1513 - DNA, 20 bp, Artificial Sequence
  Synthetic
cacagtagatgagggagcag

SEQ ID NO: 1514 - DNA, 20 bp, Artificial Sequence
  Synthetic
cacagtagatgagggagcag

SEQ ID NO: 1515 - DNA, 20 bp, Artificial Sequence
  Synthetic
cacagtagatgagggagcag

SEQ ID NO: 1516 - DNA, 20 bp, Artificial Sequence
  Synthetic
cacagtagatgagggagcag

SEQ ID NO: 1517 - DNA, 20 bp, Artificial Sequence
  Synthetic
ctcagtaacattgacaccac

SEQ ID NO: 1518 - DNA, 20 bp, Artificial Sequence
  Synthetic
ctcgactaaagcaggatttc

SEQ ID NO: 1519 - DNA, 20 bp, Artificial Sequence
  Synthetic
gcagggttaccgccatcccc

SEQ ID NO: 1520 - DNA, 20 bp, Artificial Sequence
  Synthetic
cgagacagtcgcttccactt

SEQ ID NO: 1521 - DNA, 16 bp, Artificial Sequence
  Synthetic
acacagtagatgaggg

SEQ ID NO: 1522 - DNA, 15 bp, Artificial Sequence
  Synthetic
cacagtagatgaggg

SEQ ID NO: 1523 - DNA, 14 bp, Artificial Sequence
  Synthetic
acagtagatgaggg

SEQ ID NO: 1524 - DNA, 13 bp, Artificial Sequence
  Synthetic
cagtagatgaggg

SEQ ID NO: 1525 - DNA, 12 bp, Artificial Sequence
  Synthetic
agtagatgaggg

SEQ ID NO: 1526 - DNA, 11 bp, Artificial Sequence
  Synthetic
gtagatgaggg

SEQ ID NO: 1527 - DNA, 10 bp, Artificial Sequence
  Synthetic
tagatgaggg

SEQ ID NO: 1528 - DNA, 9 bp, Artificial Sequence
  Synthetic
agatgaggg

SEQ ID NO: 1529 - DNA, 8 bp, Artificial Sequence
  Synthetic
gatgaggg

SEQ ID NO: 1530 - DNA, 7 bp, Artificial Sequence
  Synthetic
atgaggg

SEQ ID NO: 1531 - DNA, 6 bp, Artificial Sequence
  Synthetic
tgaggg

SEQ ID NO: 1532 - DNA, 16 bp, Artificial Sequence
  Synthetic
acacagtagatgaggg

SEQ ID NO: 1533 - DNA, 15 bp, Artificial Sequence
  Synthetic
cacagtagatgaggg

SEQ ID NO: 1534 - DNA, 14 bp, Artificial Sequence
  Synthetic
acagtagatgaggg

SEQ ID NO: 1535 - DNA, 13 bp, Artificial Sequence
  Synthetic
cagtagatgaggg

SEQ ID NO: 1536 - DNA, 12 bp, Artificial Sequence
  Synthetic
agtagatgaggg

SEQ ID NO: 1537 - DNA, 11 bp, Artificial Sequence
  Synthetic
gtagatgaggg

SEQ ID NO: 1538 - DNA, 10 bp, Artificial Sequence
  Synthetic
tagatgaggg

SEQ ID NO: 1539 - DNA, 9 bp, Artificial Sequence
  Synthetic
agatgaggg

SEQ ID NO: 1540 - DNA, 8 bp, Artificial Sequence
  Synthetic
gatgaggg

SEQ ID NO: 1541 - DNA, 7 bp, Artificial Sequence
  Synthetic
atgaggg

SEQ ID NO: 1542 - DNA, 6 bp, Artificial Sequence
  Synthetic
tgaggg

SEQ ID NO: 1543 - DNA, 20 bp, Artificial Sequence
  Synthetic
ctcgactaaagcaggatttc

SEQ ID NO: 1544 - DNA, 20 bp, Artificial Sequence
  Synthetic
ctcgactaaagcaggatttc

SEQ ID NO: 1545 - DNA, 20 bp, Artificial Sequence
  Synthetic
ctcagtaacattgacaccac

SEQ ID NO: 1546 - DNA, 20 bp, Artificial Sequence
  Synthetic
ctcagtaacattgacaccac

SEQ ID NO: 1547 - DNA, 20 bp, Artificial Sequence
  Synthetic
guugatctgtagcagcagct

SEQ ID NO: 1548 - DNA, 20 bp, Artificial Sequence
  Synthetic
guugatctgtagcagcagct

SEQ ID NO: 1549 - DNA, 20 bp, Artificial Sequence
  Synthetic
guugatctgtagcagcagct

SEQ ID NO: 1550 - DNA, 20 bp, Artificial Sequence
  Synthetic
guugatctgtagcagcagct

SEQ ID NO: 1551 - RNA, 19 nt, Artificial Sequence
  Synthetic
ggcaaagggcacagacuuc

SEQ ID NO: 1552 - RNA, 19 nt, Artificial Sequence
  Synthetic
ggcacaagggcacagacuc

SEQ ID NO: 1553 - RNA, 19 nt, Artificial Sequence
  Synthetic
ggacaagggcacagacuuc

SEQ ID NO: 1554 - RNA, 19 nt, Artificial Sequence
  Synthetic
ggcacaagggcacagauuc

SEQ ID NO: 1555 - RNA, 21 nt, Artificial Sequence
  Synthetic
ggcacaagggccacagacuuc

SEQ ID NO: 1556 - RNA, 21 nt, Artificial Sequence
  Synthetic
ggcacaagggcaccagacuuc

SEQ ID NO: 1557 - RNA, 21 nt, Artificial Sequence
  Synthetic
ggcacaagggcaacagacuuc

SEQ ID NO: 1558 - RNA, 21 nt, Artificial Sequence
  Synthetic
ggcacaagggcacaagacuuc

SEQ ID NO: 1559 - RNA, 21 nt, Artificial Sequence
  Synthetic
ggcaccaagggcacagacuuc

SEQ ID NO: 1560 - RNA, 20 nt, Artificial Sequence
  Synthetic
ggcacaagggcacagacuuc

SEQ ID NO: 1561 - RNA, 20 nt, Artificial Sequence
  Synthetic
ggcacaagggcacagacuuc

SEQ ID NO: 1562 - RNA, 20 nt, Artificial Sequence
  Synthetic
ggcacaagggcacagacuuc

SEQ ID NO: 1563 - RNA, 20 nt, Artificial Sequence
  Synthetic
ggcacaagggcacagacuuc

SEQ ID NO: 1564 - RNA, 20 nt, Artificial Sequence
  Synthetic
ggcacaagggcacagacuuc

SEQ ID NO: 1565 - RNA, 20 nt, Artificial Sequence
  Synthetic
ggcacaagggcacagacuuc

SEQ ID NO: 1566 - RNA, 20 nt, Artificial Sequence
  Synthetic
ggcacaagggcacagacuuc

SEQ ID NO: 1567 - RNA, 20 nt, Artificial Sequence
  Synthetic
ggcacaagggcacagacuuc

SEQ ID NO: 1568 - RNA, 20 nt, Artificial Sequence
  Synthetic
ggcacaagggcacagacuuc

SEQ ID NO: 1569 - RNA, 20 nt, Artificial Sequence
  Synthetic
ggcacaagggcacagacuuc

SEQ ID NO: 1570 - RNA, 20 nt, Artificial Sequence
  Synthetic
ggcacaagggcacagacuuc

SEQ ID NO: 1571 - RNA, 20 nt, Artificial Sequence
  Synthetic
ggcacaagggcacagacuuc

SEQ ID NO: 1572 - DNA, 20 bp, Artificial Sequence
  Synthetic
gugcacacagtagatgaggg

SEQ ID NO: 1573 - DNA, 16 bp, Artificial Sequence
  Synthetic
acacagtagatgaggg

SEQ ID NO: 1574 - DNA, 15 bp, Artificial Sequence
  Synthetic
cacagtagatgaggg

SEQ ID NO: 1575 - DNA, 14 bp, Artificial Sequence
  Synthetic
acagtagatgaggg

SEQ ID NO: 1576 - DNA, 13 bp, Artificial Sequence
  Synthetic
cagtagatgaggg

SEQ ID NO: 1577 - DNA, 12 bp, Artificial Sequence
  Synthetic
agtagatgaggg

SEQ ID NO: 1578 - DNA, 11 bp, Artificial Sequence
  Synthetic
gtagatgaggg

SEQ ID NO: 1579 - DNA, 10 bp, Artificial Sequence
  Synthetic
tagatgaggg

SEQ ID NO: 1580 - DNA, 9 bp, Artificial Sequence
  Synthetic
agatgaggg

SEQ ID NO: 1581 - DNA, 8 bp, Artificial Sequence
  Synthetic
gatgaggg

SEQ ID NO: 1582 - DNA, 7 bp, Artificial Sequence
  Synthetic
atgaggg

SEQ ID NO: 1583 - DNA, 6 bp, Artificial Sequence
  Synthetic
tgaggg

SEQ ID NO: 1584 - DNA, 2 bp, Artificial Sequence
  Synthetic
gg

SEQ ID NO: 1585 - DNA, 20 bp, Artificial Sequence
  Synthetic
gugcacacagtagatgaggg

SEQ ID NO: 1586 - DNA, 16 bp, Artificial Sequence
  Synthetic
acacagtagatgaggg

SEQ ID NO: 1587 - DNA, 15 bp, Artificial Sequence
  Synthetic
cacagtagatgaggg

SEQ ID NO: 1588 - DNA, 14 bp, Artificial Sequence
  Synthetic
acagtagatgaggg

SEQ ID NO: 1589 - DNA, 13 bp, Artificial Sequence
  Synthetic
cagtagatgaggg

SEQ ID NO: 1590 - DNA, 12 bp, Artificial Sequence
  Synthetic
agtagatgaggg

SEQ ID NO: 1591 - DNA, 11 bp, Artificial Sequence
  Synthetic
gtagatgaggg

SEQ ID NO: 1592 - DNA, 10 bp, Artificial Sequence
  Synthetic
tagatgaggg

SEQ ID NO: 1593 - DNA, 9 bp, Artificial Sequence
  Synthetic
agatgaggg

SEQ ID NO: 1594 - DNA, 8 bp, Artificial Sequence
  Synthetic
gatgaggg

SEQ ID NO: 1595 - DNA, 7 bp, Artificial Sequence
  Synthetic
atgaggg

SEQ ID NO: 1596 - DNA, 6 bp, Artificial Sequence
  Synthetic
tgaggg

SEQ ID NO: 1597 - DNA, 2 bp, Artificial Sequence
  Synthetic
gg

SEQ ID NO: 1598 - DNA, 21 bp, Artificial Sequence
  Synthetic
gugcacaacagtagatgaggg

SEQ ID NO: 1599 - DNA, 21 bp, Artificial Sequence
  Synthetic
gugcacacaagtagatgaggg

SEQ ID NO: 1600 - DNA, 21 bp, Artificial Sequence
  Synthetic
gugcacacagtaagatgaggg

SEQ ID NO: 1601 - DNA, 21 bp, Artificial Sequence
  Synthetic
gugcacacagtagaatgaggg

SEQ ID NO: 1602 - DNA, 21 bp, Artificial Sequence
  Synthetic
gugcaccacagtagatgaggg

SEQ ID NO: 1603 - DNA, 21 bp, Artificial Sequence
  Synthetic
gugcacaccagtagatgaggg

SEQ ID NO: 1604 - DNA, 21 bp, Artificial Sequence
  Synthetic
gugcaacacagtagatgaggg

SEQ ID NO: 1605 - DNA, 19 bp, Artificial Sequence
  Synthetic
gugccacagtagatgaggg

SEQ ID NO: 1606 - DNA, 19 bp, Artificial Sequence
  Synthetic
gugcaccagtagatgaggg

SEQ ID NO: 1607 - DNA, 19 bp, Artificial Sequence
  Synthetic
gugcacacgtagatgaggg

SEQ ID NO: 1608 - DNA, 19 bp, Artificial Sequence
  Synthetic
gugcacacagtgatgaggg

SEQ ID NO: 1609 - DNA, 19 bp, Artificial Sequence
  Synthetic
gugcacacagtagtgaggg

SEQ ID NO: 1610 - DNA, 19 bp, Artificial Sequence
  Synthetic
gugcaacagtagatgaggg

SEQ ID NO: 1611 - DNA, 19 bp, Artificial Sequence
  Synthetic
gugcacaagtagatgaggg

SEQ ID NO: 1612 - DNA, 16 bp, Artificial Sequence
  Synthetic
acacagtagatgaggg

SEQ ID NO: 1613 - DNA, 20 bp, Artificial Sequence
  Synthetic
gugcacacagtagatgaggg

SEQ ID NO: 1614 - DNA, 20 bp, Artificial Sequence
  Synthetic
gugcacacagtagatgaggg

SEQ ID NO: 1615 - DNA, 20 bp, Artificial Sequence
  Synthetic
gugcacacagtagatgaggg

SEQ ID NO: 1616 - DNA, 20 bp, Artificial Sequence
  Synthetic
gugcacacagtagatgaggg

SEQ ID NO: 1617 - DNA, 20 bp, Artificial Sequence
  Synthetic
gugcacacagtagatgaggg

SEQ ID NO: 1618 - DNA, 20 bp, Artificial Sequence
  Synthetic
gugcacacagtagatgaggg

SEQ ID NO: 1619 - DNA, 20 bp, Artificial Sequence
  Synthetic
gugcacacagtagatgaggg

SEQ ID NO: 1620 - DNA, 20 bp, Artificial Sequence
  Synthetic
gugcacacagtagatgaggg

SEQ ID NO: 1621 - DNA, 20 bp, Artificial Sequence
  Synthetic
gugcacacagtagatgaggg

SEQ ID NO: 1622 - DNA, 20 bp, Artificial Sequence
  Synthetic
gugcacacagtagatgaggg

SEQ ID NO: 1623 - DNA, 20 bp, Artificial Sequence
  Synthetic
gugcacacagtagatgaggg

SEQ ID NO: 1624 - DNA, 20 bp, Artificial Sequence
  Synthetic
gugcacacagtagatgaggg

SEQ ID NO: 1625 - DNA, 20 bp, Artificial Sequence
  Synthetic
gatctgaagcagcagcttct

SEQ ID NO: 1626 - DNA, 20 bp, Artificial Sequence
  Synthetic
gatctgaagcagcagcttct

SEQ ID NO: 1627 - DNA, 20 bp, Artificial Sequence
  Synthetic
gatctgaagcagcagcttct

SEQ ID NO: 1628 - DNA, 20 bp, Artificial Sequence
  Synthetic
tgatctgaagcagcagcttc

SEQ ID NO: 1629 - DNA, 20 bp, Artificial Sequence
  Synthetic
tgatctgaagcagcagcttc

SEQ ID NO: 1630 - DNA, 20 bp, Artificial Sequence
  Synthetic
tgatctgaagcagcagcttc

SEQ ID NO: 1631 - DNA, 20 bp, Artificial Sequence
  Synthetic
ttgatctgaagcagcagctt

SEQ ID NO: 1632 - DNA, 20 bp, Artificial Sequence
  Synthetic
ttgatctgaagcagcagctt

SEQ ID NO: 1633 - DNA, 20 bp, Artificial Sequence
  Synthetic
ttgatctgaagcagcagctt

SEQ ID NO: 1634 - DNA, 20 bp, Artificial Sequence
  Synthetic
gttgatctgaagcagcagct

SEQ ID NO: 1635 - DNA, 20 bp, Artificial Sequence
  Synthetic
gttgatctgaagcagcagct

SEQ ID NO: 1636 - DNA, 20 bp, Artificial Sequence
  Synthetic
gttgatctgaagcagcagct

SEQ ID NO: 1637 - DNA, 20 bp, Artificial Sequence
  Synthetic
ggttgatctgaagcagcagc

SEQ ID NO: 1638 - DNA, 20 bp, Artificial Sequence
  Synthetic
ggttgatctgaagcagcagc

SEQ ID NO: 1639 - DNA, 20 bp, Artificial Sequence
  Synthetic
ggttgatctgaagcagcagc

SEQ ID NO: 1640 - DNA, 20 bp, Artificial Sequence
  Synthetic
gggttgatctgaagcagcag

SEQ ID NO: 1641 - DNA, 20 bp, Artificial Sequence
  Synthetic
gggttgatctgaagcagcag

SEQ ID NO: 1642 - DNA, 20 bp, Artificial Sequence
  Synthetic
gggttgatctgaagcagcag

SEQ ID NO: 1643 - DNA, 20 bp, Artificial Sequence
  Synthetic
ggggttgatctgaagcagca

SEQ ID NO: 1644 - DNA, 20 bp, Artificial Sequence
  Synthetic
ggggttgatctgaagcagca

SEQ ID NO: 1645 - DNA, 20 bp, Artificial Sequence
  Synthetic
ggggttgatctgaagcagca

SEQ ID NO: 1646 - DNA, 20 bp, Artificial Sequence
  Synthetic
cggggttgatctgaagcagc

SEQ ID NO: 1647 - DNA, 20 bp, Artificial Sequence
  Synthetic
cggggttgatctgaagcagc

SEQ ID NO: 1648 - DNA, 20 bp, Artificial Sequence
  Synthetic
cggggttgatctgaagcagc

SEQ ID NO: 1649 - DNA, 20 bp, Artificial Sequence
  Synthetic
gatctgaagcagcagcuucu

SEQ ID NO: 1650 - DNA, 20 bp, Artificial Sequence
  Synthetic
gatctgaagcagcagcuucu

SEQ ID NO: 1651 - DNA, 20 bp, Artificial Sequence
  Synthetic
gatctgaagcagcagcuucu

SEQ ID NO: 1652 - DNA, 20 bp, Artificial Sequence
  Synthetic
tgatctgaagcagcagcuuc

SEQ ID NO: 1653 - DNA, 20 bp, Artificial Sequence
  Synthetic
tgatctgaagcagcagcuuc

SEQ ID NO: 1654 - DNA, 20 bp, Artificial Sequence
  Synthetic
tgatctgaagcagcagcuuc

SEQ ID NO: 1655 - DNA, 20 bp, Artificial Sequence
  Synthetic
ttgatctgaagcagcagcuu

SEQ ID NO: 1656 - DNA, 20 bp, Artificial Sequence
  Synthetic
ttgatctgaagcagcagcuu

SEQ ID NO: 1657 - DNA, 20 bp, Artificial Sequence
  Synthetic
ttgatctgaagcagcagcuu

SEQ ID NO: 1658 - DNA, 20 bp, Artificial Sequence
  Synthetic
gttgatctgaagcagcagcu

SEQ ID NO: 1659 - DNA, 20 bp, Artificial Sequence
  Synthetic
gttgatctgaagcagcagcu

SEQ ID NO: 1660 - DNA, 20 bp, Artificial Sequence
  Synthetic
gttgatctgaagcagcagcu

SEQ ID NO: 1661 - DNA, 20 bp, Artificial Sequence
  Synthetic
ggttgatctgaagcagcagc

SEQ ID NO: 1662 - DNA, 20 bp, Artificial Sequence
  Synthetic
ggttgatctgaagcagcagc

SEQ ID NO: 1663 - DNA, 20 bp, Artificial Sequence
  Synthetic
ggttgatctgaagcagcagc

SEQ ID NO: 1664 - DNA, 20 bp, Artificial Sequence
  Synthetic
gggttgatctgaagcagcag

SEQ ID NO: 1665 - DNA, 20 bp, Artificial Sequence
  Synthetic
gggttgatctgaagcagcag

SEQ ID NO: 1666 - DNA, 20 bp, Artificial Sequence
  Synthetic
gggttgatctgaagcagcag

SEQ ID NO: 1667 - DNA, 20 bp, Artificial Sequence
  Synthetic
ggggttgatctgaagcagca

SEQ ID NO: 1668 - DNA, 20 bp, Artificial Sequence
  Synthetic
ggggttgatctgaagcagca

SEQ ID NO: 1669 - DNA, 20 bp, Artificial Sequence
  Synthetic
ggggttgatctgaagcagca

SEQ ID NO: 1670 - DNA, 20 bp, Artificial Sequence
  Synthetic
cggggttgatctgaagcagc

SEQ ID NO: 1671 - DNA, 20 bp, Artificial Sequence
  Synthetic
cggggttgatctgaagcagc

SEQ ID NO: 1672 - DNA, 20 bp, Artificial Sequence
  Synthetic
cggggttgatctgaagcagc

SEQ ID NO: 1673 - DNA, 20 bp, Artificial Sequence
  Synthetic
gaucugaagcagcagcttct

SEQ ID NO: 1674 - DNA, 20 bp, Artificial Sequence
  Synthetic
gaucugaagcagcagcttct

SEQ ID NO: 1675 - DNA, 20 bp, Artificial Sequence
  Synthetic
gaucugaagcagcagcttct

SEQ ID NO: 1676 - DNA, 20 bp, Artificial Sequence
  Synthetic
ugauctgaagcagcagcttc

SEQ ID NO: 1677 - DNA, 20 bp, Artificial Sequence
  Synthetic
ugauctgaagcagcagcttc

SEQ ID NO: 1678 - DNA, 20 bp, Artificial Sequence
  Synthetic
ugauctgaagcagcagcttc

SEQ ID NO: 1679 - DNA, 20 bp, Artificial Sequence
  Synthetic
uugauctgaagcagcagctt

SEQ ID NO: 1680 - DNA, 20 bp, Artificial Sequence
  Synthetic
uugauctgaagcagcagctt

SEQ ID NO: 1681 - DNA, 20 bp, Artificial Sequence
  Synthetic
uugauctgaagcagcagctt

SEQ ID NO: 1682 - DNA, 20 bp, Artificial Sequence
  Synthetic
guugatctgaagcagcagct

SEQ ID NO: 1683 - DNA, 20 bp, Artificial Sequence
  Synthetic
guugatctgaagcagcagct

SEQ ID NO: 1684 - DNA, 20 bp, Artificial Sequence
  Synthetic
guugatctgaagcagcagct

SEQ ID NO: 1685 - DNA, 20 bp, Artificial Sequence
  Synthetic
gguugatctgaagcagcagc

SEQ ID NO: 1686 - DNA, 20 bp, Artificial Sequence
  Synthetic
gguugatctgaagcagcagc

SEQ ID NO: 1687 - DNA, 20 bp, Artificial Sequence
  Synthetic
gguugatctgaagcagcagc

SEQ ID NO: 1688 - DNA, 20 bp, Artificial Sequence
  Synthetic
ggguugatctgaagcagcag

SEQ ID NO: 1689 - DNA, 20 bp, Artificial Sequence
  Synthetic
ggguugatctgaagcagcag

SEQ ID NO: 1690 - DNA, 20 bp, Artificial Sequence
  Synthetic
ggguugatctgaagcagcag

SEQ ID NO: 1691 - DNA, 20 bp, Artificial Sequence
  Synthetic
ggggutgatctgaagcagca

SEQ ID NO: 1692 - DNA, 20 bp, Artificial Sequence
  Synthetic
ggggutgatctgaagcagca

SEQ ID NO: 1693 - DNA, 20 bp, Artificial Sequence
  Synthetic
ggggutgatctgaagcagca

SEQ ID NO: 1694 - DNA, 20 bp, Artificial Sequence
  Synthetic
cggggttgatctgaagcagc

SEQ ID NO: 1695 - DNA, 20 bp, Artificial Sequence
  Synthetic
cggggttgatctgaagcagc

SEQ ID NO: 1696 - DNA, 20 bp, Artificial Sequence
  Synthetic
cggggttgatctgaagcagc

SEQ ID NO: 1697 - DNA, 20 bp, Artificial Sequence
  Synthetic
gatctgtagcagcagcttct

SEQ ID NO: 1698 - DNA, 20 bp, Artificial Sequence
  Synthetic
gatctgtagcagcagcttct

SEQ ID NO: 1699 - DNA, 20 bp, Artificial Sequence
  Synthetic
gatctgtagcagcagcttct

SEQ ID NO: 1700 - DNA, 20 bp, Artificial Sequence
  Synthetic
tgatctgtagcagcagcttc

SEQ ID NO: 1701 - DNA, 20 bp, Artificial Sequence
  Synthetic
tgatctgtagcagcagcttc

SEQ ID NO: 1702 - DNA, 20 bp, Artificial Sequence
  Synthetic
tgatctgtagcagcagcttc

SEQ ID NO: 1703 - DNA, 20 bp, Artificial Sequence
  Synthetic
ttgatctgtagcagcagctt

SEQ ID NO: 1704 - DNA, 20 bp, Artificial Sequence
  Synthetic
ttgatctgtagcagcagctt

SEQ ID NO: 1705 - DNA, 20 bp, Artificial Sequence
  Synthetic
ttgatctgtagcagcagctt

SEQ ID NO: 1706 - DNA, 20 bp, Artificial Sequence
  Synthetic
gttgatctgtagcagcagct

SEQ ID NO: 1707 - DNA, 20 bp, Artificial Sequence
  Synthetic
gttgatctgtagcagcagct

SEQ ID NO: 1708 - DNA, 20 bp, Artificial Sequence
  Synthetic
ggttgatctgtagcagcagc

SEQ ID NO: 1709 - DNA, 20 bp, Artificial Sequence
  Synthetic
ggttgatctgtagcagcagc

SEQ ID NO: 1710 - DNA, 20 bp, Artificial Sequence
  Synthetic
ggttgatctgtagcagcagc

SEQ ID NO: 1711 - DNA, 20 bp, Artificial Sequence
  Synthetic
gggttgatctgtagcagcag

SEQ ID NO: 1712 - DNA, 20 bp, Artificial Sequence
  Synthetic
gggttgatctgtagcagcag

SEQ ID NO: 1713 - DNA, 20 bp, Artificial Sequence
  Synthetic
gggttgatctgtagcagcag

SEQ ID NO: 1714 - DNA, 20 bp, Artificial Sequence
  Synthetic
ggggttgatctgtagcagca

SEQ ID NO: 1715 - DNA, 20 bp, Artificial Sequence
  Synthetic
ggggttgatctgtagcagca

SEQ ID NO: 1716 - DNA, 20 bp, Artificial Sequence
  Synthetic
ggggttgatctgtagcagca

SEQ ID NO: 1717 - DNA, 20 bp, Artificial Sequence
  Synthetic
cggggttgatctgtagcagc

SEQ ID NO: 1718 - DNA, 20 bp, Artificial Sequence
  Synthetic
cggggttgatctgtagcagc

SEQ ID NO: 1719 - DNA, 20 bp, Artificial Sequence
  Synthetic
cggggttgatctgtagcagc

SEQ ID NO: 1720 - DNA, 20 bp, Artificial Sequence
  Synthetic
gatctgtagcagcagcuucu

SEQ ID NO: 1721 - DNA, 20 bp, Artificial Sequence
  Synthetic
gatctgtagcagcagcuucu

SEQ ID NO: 1722 - DNA, 20 bp, Artificial Sequence
  Synthetic
gatctgtagcagcagcuucu

SEQ ID NO: 1723 - DNA, 20 bp, Artificial Sequence
  Synthetic
tgatctgtagcagcagcuuc

SEQ ID NO: 1724 - DNA, 20 bp, Artificial Sequence
  Synthetic
tgatctgtagcagcagcuuc

SEQ ID NO: 1725 - DNA, 20 bp, Artificial Sequence
  Synthetic
tgatctgtagcagcagcuuc

SEQ ID NO: 1726 - DNA, 20 bp, Artificial Sequence
  Synthetic
ttgatctgtagcagcagcuu

SEQ ID NO: 1727 - DNA, 20 bp, Artificial Sequence
  Synthetic
ttgatctgtagcagcagcuu

SEQ ID NO: 1728 - DNA, 20 bp, Artificial Sequence
  Synthetic
ttgatctgtagcagcagcuu

SEQ ID NO: 1729 - DNA, 20 bp, Artificial Sequence
  Synthetic
gttgatctgtagcagcagcu

SEQ ID NO: 1730 - DNA, 20 bp, Artificial Sequence
  Synthetic
gttgatctgtagcagcagcu

SEQ ID NO: 1731 - DNA, 20 bp, Artificial Sequence
  Synthetic
gttgatctgtagcagcagcu

SEQ ID NO: 1732 - DNA, 20 bp, Artificial Sequence
  Synthetic
ggttgatctgtagcagcagc

SEQ ID NO: 1733 - DNA, 20 bp, Artificial Sequence
  Synthetic
ggttgatctgtagcagcagc

SEQ ID NO: 1734 - DNA, 20 bp, Artificial Sequence
  Synthetic
ggttgatctgtagcagcagc

SEQ ID NO: 1735 - DNA, 20 bp, Artificial Sequence
  Synthetic
gggttgatctgtagcagcag

SEQ ID NO: 1736 - DNA, 20 bp, Artificial Sequence
  Synthetic
gggttgatctgtagcagcag

SEQ ID NO: 1737 - DNA, 20 bp, Artificial Sequence
  Synthetic
gggttgatctgtagcagcag

SEQ ID NO: 1738 - DNA, 20 bp, Artificial Sequence
  Synthetic
ggggttgatctgtagcagca

SEQ ID NO: 1739 - DNA, 20 bp, Artificial Sequence
  Synthetic
ggggttgatctgtagcagca

SEQ ID NO: 1740 - DNA, 20 bp, Artificial Sequence
  Synthetic
ggggttgatctgtagcagca

SEQ ID NO: 1741 - DNA, 20 bp, Artificial Sequence
  Synthetic
cggggttgatctgtagcagc

SEQ ID NO: 1742 - DNA, 20 bp, Artificial Sequence
  Synthetic
cggggttgatctgtagcagc

SEQ ID NO: 1743 - DNA, 20 bp, Artificial Sequence
  Synthetic
cggggttgatctgtagcagc

SEQ ID NO: 1744 - DNA, 20 bp, Artificial Sequence
  Synthetic
gaucugtagcagcagcttct

SEQ ID NO: 1745 - DNA, 20 bp, Artificial Sequence
  Synthetic
gaucugtagcagcagcttct

SEQ ID NO: 1746 - DNA, 20 bp, Artificial Sequence
  Synthetic
gaucugtagcagcagcttct

SEQ ID NO: 1747 - DNA, 20 bp, Artificial Sequence
  Synthetic
ugauctgtagcagcagcttc

SEQ ID NO: 1748 - DNA, 20 bp, Artificial Sequence
  Synthetic
ugauctgtagcagcagcttc

SEQ ID NO: 1749 - DNA, 20 bp, Artificial Sequence
  Synthetic
ugauctgtagcagcagcttc

SEQ ID NO: 1750 - DNA, 20 bp, Artificial Sequence
  Synthetic
uugauctgtagcagcagctt

SEQ ID NO: 1751 - DNA, 20 bp, Artificial Sequence
  Synthetic
uugauctgtagcagcagctt

SEQ ID NO: 1752 - DNA, 20 bp, Artificial Sequence
  Synthetic
uugauctgtagcagcagctt

SEQ ID NO: 1753 - DNA, 20 bp, Artificial Sequence
  Synthetic
gguugatctgtagcagcagc

SEQ ID NO: 1754 - DNA, 20 bp, Artificial Sequence
  Synthetic
gguugatctgtagcagcagc

SEQ ID NO: 1755 - DNA, 20 bp, Artificial Sequence
  Synthetic
gguugatctgtagcagcagc

SEQ ID NO: 1756 - DNA, 20 bp, Artificial Sequence
  Synthetic
ggguugatctgtagcagcag

SEQ ID NO: 1757 - DNA, 20 bp, Artificial Sequence
  Synthetic
ggguugatctgtagcagcag

SEQ ID NO: 1758 - DNA, 20 bp, Artificial Sequence
  Synthetic
ggguugatctgtagcagcag

SEQ ID NO: 1759 - DNA, 20 bp, Artificial Sequence
  Synthetic
ggggutgatctgtagcagca

SEQ ID NO: 1760 - DNA, 20 bp, Artificial Sequence
  Synthetic
ggggutgatctgtagcagca

SEQ ID NO: 1761 - DNA, 20 bp, Artificial Sequence
  Synthetic
ggggutgatctgtagcagca

SEQ ID NO: 1762 - DNA, 20 bp, Artificial Sequence
  Synthetic
cggggttgatctgtagcagc

SEQ ID NO: 1763 - DNA, 20 bp, Artificial Sequence
  Synthetic
cggggttgatctgtagcagc

SEQ ID NO: 1764 - DNA, 20 bp, Artificial Sequence
  Synthetic
cggggttgatctgtagcagc

SEQ ID NO: 1765 - DNA, 20 bp, Artificial Sequence
  Synthetic
cggggttgatctgtagcagc

SEQ ID NO: 1766 - DNA, 20 bp, Artificial Sequence
  Synthetic
cggggttgatctgtagcagc

SEQ ID NO: 1767 - DNA, 20 bp, Artificial Sequence
  Synthetic
cggggttgatctgtagcagc

SEQ ID NO: 1768 - DNA, 20 bp, Artificial Sequence
  Synthetic
cggggttgatctgtagcagc

SEQ ID NO: 1769 - DNA, 20 bp, Artificial Sequence
  Synthetic
cggggttgatctgtagcagc

SEQ ID NO: 1770 - DNA, 20 bp, Artificial Sequence
  Synthetic
cggggttgatctgtagcagc

SEQ ID NO: 1771 - DNA, 20 bp, Artificial Sequence
  Synthetic
accgcgaccctctggacagg

SEQ ID NO: 1772 - DNA, 20 bp, Artificial Sequence
  Synthetic
accgcgaccctctggacagg

SEQ ID NO: 1773 - DNA, 20 bp, Artificial Sequence
  Synthetic
accgccatccccgccguagc

SEQ ID NO: 1774 - DNA, 20 bp, Artificial Sequence
  Synthetic
accgccatccccgccguagc

SEQ ID NO: 1775 - DNA, 20 bp, Artificial Sequence
  Synthetic
accgccatccccgccguagc

SEQ ID NO: 1776 - DNA, 20 bp, Artificial Sequence
  Synthetic
accgccatccccgccguagc

SEQ ID NO: 1777 - DNA, 20 bp, Artificial Sequence
  Synthetic
accgccatccccgccguagc

SEQ ID NO: 1778 - DNA, 20 bp, Artificial Sequence
  Synthetic
gttaccgccatccccgccgu

SEQ ID NO: 1779 - DNA, 20 bp, Artificial Sequence
  Synthetic
gttaccgccatccccgccgu

SEQ ID NO: 1780 - DNA, 20 bp, Artificial Sequence
  Synthetic
gttaccgccatccccgccgu

SEQ ID NO: 1781 - DNA, 20 bp, Artificial Sequence
  Synthetic
gttaccgccatccccgccgu

SEQ ID NO: 1782 - DNA, 20 bp, Artificial Sequence
  Synthetic
gttaccgccatccccgccgu

SEQ ID NO: 1783 - DNA, 20 bp, Artificial Sequence
  Synthetic
ggctctgggttgctggguca

SEQ ID NO: 1784 - DNA, 20 bp, Artificial Sequence
  Synthetic
ggctctgggttgctggguca

SEQ ID NO: 1785 - DNA, 20 bp, Artificial Sequence
  Synthetic
ggctctgggttgctggguca

SEQ ID NO: 1786 - DNA, 20 bp, Artificial Sequence
  Synthetic
ggctctgggttgctggguca

SEQ ID NO: 1787 - DNA, 20 bp, Artificial Sequence
  Synthetic
ggtguccctcatgggcucug

SEQ ID NO: 1788 - DNA, 20 bp, Artificial Sequence
  Synthetic
ggtguccctcatgggcucug

SEQ ID NO: 1789 - DNA, 20 bp, Artificial Sequence
  Synthetic
ggtguccctcatgggcucug

SEQ ID NO: 1790 - DNA, 20 bp, Artificial Sequence
  Synthetic
ggtguccctcatgggcucug

SEQ ID NO: 1791 - DNA, 20 bp, Artificial Sequence
  Synthetic
accgcgaccctctggacagg

SEQ ID NO: 1792 - DNA, 20 bp, Artificial Sequence
  Synthetic
accgcgaccctctggacagg

SEQ ID NO: 1793 - DNA, 20 bp, Artificial Sequence
  Synthetic
accgcgaccctctggacagg

SEQ ID NO: 1794 - DNA, 20 bp, Artificial Sequence
  Synthetic
accgcgaccctctggacagg

SEQ ID NO: 1795 - DNA, 20 bp, Artificial Sequence
  Synthetic
accgcgaccctctggacagg

SEQ ID NO: 1796 - DNA, 20 bp, Artificial Sequence
  Synthetic
accgcgaccctctggacagg

SEQ ID NO: 1797 - DNA, 20 bp, Artificial Sequence
  Synthetic
accgcgaccctctggacagg

SEQ ID NO: 1798 - DNA, 20 bp, Artificial Sequence
  Synthetic
accgcgaccctctggacagg

SEQ ID NO: 1799 - DNA, 20 bp, Artificial Sequence
  Synthetic
accgcgaccctctggacagg

SEQ ID NO: 1800 - DNA, 20 bp, Artificial Sequence
  Synthetic
accgcgaccctctggacagg

SEQ ID NO: 1801 - DNA, 20 bp, Artificial Sequence
  Synthetic
accgcgaccctctggacagg

SEQ ID NO: 1802 - DNA, 20 bp, Artificial Sequence
  Synthetic
accgcgaccctctggacagg

SEQ ID NO: 1803 - DNA, 20 bp, Artificial Sequence
  Synthetic
accgcgaccctctggacagg

SEQ ID NO: 1804 - DNA, 20 bp, Artificial Sequence
  Synthetic
accgcgaccctctggacagg

SEQ ID NO: 1805 - DNA, 20 bp, Artificial Sequence
  Synthetic
accgcgaccctctggacagg

SEQ ID NO: 1806 - DNA, 20 bp, Artificial Sequence
  Synthetic
accgcgaccctctggacagg

SEQ ID NO: 1807 - DNA, 20 bp, Artificial Sequence
  Synthetic
accgcgaccctctggacagg

SEQ ID NO: 1808 - DNA, 20 bp, Artificial Sequence
  Synthetic
accgcgaccctctggacagg

SEQ ID NO: 1809 - DNA, 20 bp, Artificial Sequence
  Synthetic
accgcgaccctctggacagg

SEQ ID NO: 1810 - DNA, 20 bp, Artificial Sequence
  Synthetic
accgcgaccctctggacagg

SEQ ID NO: 1811 - DNA, 20 bp, Artificial Sequence
  Synthetic
accgcgaccctctggacagg

SEQ ID NO: 1812 - DNA, 20 bp, Artificial Sequence
  Synthetic
accgcgaccctctggacagg

SEQ ID NO: 1813 - DNA, 20 bp, Artificial Sequence
  Synthetic
accgcgaccctctggacagg

SEQ ID NO: 1814 - DNA, 20 bp, Artificial Sequence
  Synthetic
accgcgaccctctggacagg

SEQ ID NO: 1815 - DNA, 20 bp, Artificial Sequence
  Synthetic
accgcgaccctctggacagg

SEQ ID NO: 1816 - DNA, 20 bp, Artificial Sequence
  Synthetic
accgcgaccctctggacagg

SEQ ID NO: 1817 - DNA, 20 bp, Artificial Sequence
  Synthetic
accgcgaccctctggacagg

SEQ ID NO: 1818 - DNA, 20 bp, Artificial Sequence
  Synthetic
accgcgaccctctggacagg

SEQ ID NO: 1819 - DNA, 20 bp, Artificial Sequence
  Synthetic
accgcgaccctctggacagg

SEQ ID NO: 1820 - DNA, 20 bp, Artificial Sequence
  Synthetic
accgcgaccctctggacagg

SEQ ID NO: 1821 - DNA, 20 bp, Artificial Sequence
  Synthetic
accgcgaccctctggacagg

SEQ ID NO: 1822 - DNA, 20 bp, Artificial Sequence
  Synthetic
accgcgaccctctggacagg

SEQ ID NO: 1823 - DNA, 20 bp, Artificial Sequence
  Synthetic
accgcgaccctctggacagg

SEQ ID NO: 1824 - DNA, 20 bp, Artificial Sequence
  Synthetic
accgcgaccctctggacagg

SEQ ID NO: 1825 - DNA, 20 bp, Artificial Sequence
  Synthetic
accgcgaccctctggacagg

SEQ ID NO: 1826 - DNA, 20 bp, Artificial Sequence
  Synthetic
accgcgaccctctggacagg

SEQ ID NO: 1827 - DNA, 20 bp, Artificial Sequence
  Synthetic
accgcgaccctctggacagg

SEQ ID NO: 1828 - DNA, 20 bp, Artificial Sequence
  Synthetic
accgcgaccctctggacagg

SEQ ID NO: 1829 - DNA, 20 bp, Artificial Sequence
  Synthetic
accgcgaccctctggacagg

SEQ ID NO: 1830 - DNA, 20 bp, Artificial Sequence
  Synthetic
accgcgaccctctggacagg

SEQ ID NO: 1831 - DNA, 20 bp, Artificial Sequence
  Synthetic
accgcgaccctctggacagg

SEQ ID NO: 1832 - DNA, 20 bp, Artificial Sequence
  Synthetic
accgcgaccctctggacagg

SEQ ID NO: 1833 - DNA, 20 bp, Artificial Sequence
  Synthetic
accgcgaccctctggacagg

SEQ ID NO: 1834 - DNA, 20 bp, Artificial Sequence
  Synthetic
accgcgaccctctggacagg

SEQ ID NO: 1835 - DNA, 20 bp, Artificial Sequence
  Synthetic
accgcgaccctctggacagg

SEQ ID NO: 1836 - DNA, 20 bp, Artificial Sequence
  Synthetic
accgcgaccctctggacagg

SEQ ID NO: 1837 - DNA, 20 bp, Artificial Sequence
  Synthetic
accgcgaccctctggacagg

SEQ ID NO: 1838 - DNA, 20 bp, Artificial Sequence
  Synthetic
accgcgaccctctggacagg

SEQ ID NO: 1839 - DNA, 20 bp, Artificial Sequence
  Synthetic
accgcgaccctctggacagg

SEQ ID NO: 1840 - DNA, 20 bp, Artificial Sequence
  Synthetic
accgcgaccctctggacagg

SEQ ID NO: 1841 - DNA, 20 bp, Artificial Sequence
  Synthetic
accgcgaccctctggacagg

SEQ ID NO: 1842 - DNA, 20 bp, Artificial Sequence
  Synthetic
accgcgaccctctggacagg

SEQ ID NO: 1843 - DNA, 20 bp, Artificial Sequence
  Synthetic
accgcgaccctctggacagg

SEQ ID NO: 1844 - DNA, 20 bp, Artificial Sequence
  Synthetic
accgcgaccctctggacagg

SEQ ID NO: 1845 - DNA, 20 bp, Artificial Sequence
  Synthetic
accgcgaccctctggacagg

SEQ ID NO: 1846 - DNA, 20 bp, Artificial Sequence
  Synthetic
accgugaccctctggacagg

SEQ ID NO: 1847 - DNA, 20 bp, Artificial Sequence
  Synthetic
accgugaccctctggacagg

SEQ ID NO: 1848 - DNA, 20 bp, Artificial Sequence
  Synthetic
accgugaccctctggacagg

SEQ ID NO: 1849 - DNA, 20 bp, Artificial Sequence
  Synthetic
accgugaccctctggacagg

SEQ ID NO: 1850 - DNA, 20 bp, Artificial Sequence
  Synthetic
accgugaccctctggacagg

SEQ ID NO: 1851 - DNA, 20 bp, Artificial Sequence
  Synthetic
accgugaccctctggacagg

SEQ ID NO: 1852 - DNA, 20 bp, Artificial Sequence
  Synthetic
accgugaccctctggacagg

SEQ ID NO: 1853 - DNA, 20 bp, Artificial Sequence
  Synthetic
accgugaccctctggacagg

SEQ ID NO: 1854 - DNA, 20 bp, Artificial Sequence
  Synthetic
accgugaccctctggacagg

SEQ ID NO: 1855 - DNA, 20 bp, Artificial Sequence
  Synthetic
accgugaccctctggacagg

SEQ ID NO: 1856 - DNA, 20 bp, Artificial Sequence
  Synthetic
accgugaccctctggacagg

SEQ ID NO: 1857 - DNA, 20 bp, Artificial Sequence
  Synthetic
accgugaccctctggacagg

SEQ ID NO: 1858 - DNA, 20 bp, Artificial Sequence
  Synthetic
accgugaccctctggacagg

SEQ ID NO: 1859 - DNA, 20 bp, Artificial Sequence
  Synthetic
accgugaccctctggacagg

SEQ ID NO: 1860 - DNA, 20 bp, Artificial Sequence
  Synthetic
accgugaccctctggacagg

SEQ ID NO: 1861 - DNA, 20 bp, Artificial Sequence
  Synthetic
guugatctgtagcagcagct

SEQ ID NO: 1862 - DNA, 20 bp, Artificial Sequence
  Synthetic
guugatctgtagcagcagct

SEQ ID NO: 1863 - DNA, 20 bp, Artificial Sequence
  Synthetic
guugatctgtagcagcagct

SEQ ID NO: 1864 - DNA, 20 bp, Artificial Sequence
  Synthetic
guugatctgtagcagcagct

SEQ ID NO: 1865 - DNA, 20 bp, Artificial Sequence
  Synthetic
agaggacgccgtgcagggct

SEQ ID NO: 1866 - DNA, 20 bp, Artificial Sequence
  Synthetic
agaggacgccgtgcagggct

SEQ ID NO: 1867 - DNA, 20 bp, Artificial Sequence
  Synthetic
agaggacgccgtgcagggct

SEQ ID NO: 1868 - DNA, 20 bp, Artificial Sequence
  Synthetic
tagaggacgccgtgcagggc

SEQ ID NO: 1869 - DNA, 20 bp, Artificial Sequence
  Synthetic
tagaggacgccgtgcagggc

SEQ ID NO: 1870 - DNA, 20 bp, Artificial Sequence
  Synthetic
tagaggacgccgtgcagggc

SEQ ID NO: 1871 - DNA, 20 bp, Artificial Sequence
  Synthetic
atagaggacgccgtgcaggg

SEQ ID NO: 1872 - DNA, 20 bp, Artificial Sequence
  Synthetic
atagaggacgccgtgcaggg

SEQ ID NO: 1873 - DNA, 20 bp, Artificial Sequence
  Synthetic
atagaggacgccgtgcaggg

SEQ ID NO: 1874 - DNA, 20 bp, Artificial Sequence
  Synthetic
catagaggacgccgtgcagg

SEQ ID NO: 1875 - DNA, 20 bp, Artificial Sequence
  Synthetic
catagaggacgccgtgcagg

SEQ ID NO: 1876 - DNA, 20 bp, Artificial Sequence
  Synthetic
catagaggacgccgtgcagg

SEQ ID NO: 1877 - DNA, 20 bp, Artificial Sequence
  Synthetic
acatagaggacgccgtgcag

SEQ ID NO: 1878 - DNA, 20 bp, Artificial Sequence
  Synthetic
acatagaggacgccgtgcag

SEQ ID NO: 1879 - DNA, 20 bp, Artificial Sequence
  Synthetic
acatagaggacgccgtgcag

SEQ ID NO: 1880 - DNA, 20 bp, Artificial Sequence
  Synthetic
cacatagaggacgccgtgca

SEQ ID NO: 1881 - DNA, 20 bp, Artificial Sequence
  Synthetic
cacatagaggacgccgtgca

SEQ ID NO: 1882 - DNA, 20 bp, Artificial Sequence
  Synthetic
cacatagaggacgccgtgca

SEQ ID NO: 1883 - DNA, 20 bp, Artificial Sequence
  Synthetic
gcacatagaggacgccgtgc

SEQ ID NO: 1884 - DNA, 20 bp, Artificial Sequence
  Synthetic
gcacatagaggacgccgtgc

SEQ ID NO: 1885 - DNA, 20 bp, Artificial Sequence
  Synthetic
gcacatagaggacgccgtgc

SEQ ID NO: 1886 - DNA, 20 bp, Artificial Sequence
  Synthetic
agaggacgccgtgcagggcu

SEQ ID NO: 1887 - DNA, 20 bp, Artificial Sequence
  Synthetic
agaggacgccgtgcagggcu

SEQ ID NO: 1888 - DNA, 20 bp, Artificial Sequence
  Synthetic
agaggacgccgtgcagggcu

SEQ ID NO: 1889 - DNA, 20 bp, Artificial Sequence
  Synthetic
tagaggacgccgtgcagggc

SEQ ID NO: 1890 - DNA, 20 bp, Artificial Sequence
  Synthetic
tagaggacgccgtgcagggc

SEQ ID NO: 1891 - DNA, 20 bp, Artificial Sequence
  Synthetic
tagaggacgccgtgcagggc

SEQ ID NO: 1892 - DNA, 20 bp, Artificial Sequence
  Synthetic
atagaggacgccgtgcaggg

SEQ ID NO: 1893 - DNA, 20 bp, Artificial Sequence
  Synthetic
atagaggacgccgtgcaggg

SEQ ID NO: 1894 - DNA, 20 bp, Artificial Sequence
  Synthetic
atagaggacgccgtgcaggg

SEQ ID NO: 1895 - DNA, 20 bp, Artificial Sequence
  Synthetic
catagaggacgccgtgcagg

SEQ ID NO: 1896 - DNA, 20 bp, Artificial Sequence
  Synthetic
catagaggacgccgtgcagg

SEQ ID NO: 1897 - DNA, 20 bp, Artificial Sequence
  Synthetic
catagaggacgccgtgcagg

SEQ ID NO: 1898 - DNA, 20 bp, Artificial Sequence
  Synthetic
acatagaggacgccgugcag

SEQ ID NO: 1899 - DNA, 20 bp, Artificial Sequence
  Synthetic
acatagaggacgccgugcag

SEQ ID NO: 1900 - DNA, 20 bp, Artificial Sequence
  Synthetic
acatagaggacgccgugcag

SEQ ID NO: 1901 - DNA, 20 bp, Artificial Sequence
  Synthetic
cacatagaggacgccgugca

SEQ ID NO: 1902 - DNA, 20 bp, Artificial Sequence
  Synthetic
cacatagaggacgccgugca

SEQ ID NO: 1903 - DNA, 20 bp, Artificial Sequence
  Synthetic
cacatagaggacgccgugca

SEQ ID NO: 1904 - DNA, 20 bp, Artificial Sequence
  Synthetic
gcacatagaggacgccgugc

SEQ ID NO: 1905 - DNA, 20 bp, Artificial Sequence
  Synthetic
gcacatagaggacgccgugc

SEQ ID NO: 1906 - DNA, 20 bp, Artificial Sequence
  Synthetic
gcacatagaggacgccgugc

SEQ ID NO: 1907 - DNA, 20 bp, Artificial Sequence
  Synthetic
agaggacgccgtgcagggct

SEQ ID NO: 1908 - DNA, 20 bp, Artificial Sequence
  Synthetic
agaggacgccgtgcagggct

SEQ ID NO: 1909 - DNA, 20 bp, Artificial Sequence
  Synthetic
agaggacgccgtgcagggct

SEQ ID NO: 1910 - DNA, 20 bp, Artificial Sequence
  Synthetic
uagaggacgccgtgcagggc

SEQ ID NO: 1911 - DNA, 20 bp, Artificial Sequence
  Synthetic
uagaggacgccgtgcagggc

SEQ ID NO: 1912 - DNA, 20 bp, Artificial Sequence
  Synthetic
uagaggacgccgtgcagggc

SEQ ID NO: 1913 - DNA, 20 bp, Artificial Sequence
  Synthetic
auagaggacgccgtgcaggg

SEQ ID NO: 1914 - DNA, 20 bp, Artificial Sequence
  Synthetic
auagaggacgccgtgcaggg

SEQ ID NO: 1915 - DNA, 20 bp, Artificial Sequence
  Synthetic
auagaggacgccgtgcaggg

SEQ ID NO: 1916 - DNA, 20 bp, Artificial Sequence
  Synthetic
cauagaggacgccgtgcagg

SEQ ID NO: 1917 - DNA, 20 bp, Artificial Sequence
  Synthetic
cauagaggacgccgtgcagg

SEQ ID NO: 1918 - DNA, 20 bp, Artificial Sequence
  Synthetic
cauagaggacgccgtgcagg

SEQ ID NO: 1919 - DNA, 20 bp, Artificial Sequence
  Synthetic
acauagaggacgccgtgcag

SEQ ID NO: 1920 - DNA, 20 bp, Artificial Sequence
  Synthetic
acauagaggacgccgtgcag

SEQ ID NO: 1921 - DNA, 20 bp, Artificial Sequence
  Synthetic
acauagaggacgccgtgcag

SEQ ID NO: 1922 - DNA, 20 bp, Artificial Sequence
  Synthetic
cacauagaggacgccgtgca

SEQ ID NO: 1923 - DNA, 20 bp, Artificial Sequence
  Synthetic
cacauagaggacgccgtgca

SEQ ID NO: 1924 - DNA, 20 bp, Artificial Sequence
  Synthetic
cacauagaggacgccgtgca

SEQ ID NO: 1925 - DNA, 20 bp, Artificial Sequence
  Synthetic
gcacatagaggacgccgtgc

SEQ ID NO: 1926 - DNA, 20 bp, Artificial Sequence
  Synthetic
gcacatagaggacgccgtgc

SEQ ID NO: 1927 - DNA, 20 bp, Artificial Sequence
  Synthetic
gcacatagaggacgccgtgc

SEQ ID NO: 1928 - DNA, 20 bp, Artificial Sequence
  Synthetic
agaggacgccgtgcagggct

SEQ ID NO: 1929 - DNA, 20 bp, Artificial Sequence
  Synthetic
agaggacgccgtgcagggct

SEQ ID NO: 1930 - DNA, 20 bp, Artificial Sequence
  Synthetic
agaggacgccgtgcagggct

SEQ ID NO: 1931 - DNA, 20 bp, Artificial Sequence
  Synthetic
tagaggacgccgtgcagggc

SEQ ID NO: 1932 - DNA, 20 bp, Artificial Sequence
  Synthetic
tagaggacgccgtgcagggc

SEQ ID NO: 1933 - DNA, 20 bp, Artificial Sequence
  Synthetic
tagaggacgccgtgcagggc

SEQ ID NO: 1934 - DNA, 20 bp, Artificial Sequence
  Synthetic
atagaggacgccgtgcaggg

SEQ ID NO: 1935 - DNA, 20 bp, Artificial Sequence
  Synthetic
atagaggacgccgtgcaggg

SEQ ID NO: 1936 - DNA, 20 bp, Artificial Sequence
  Synthetic
atagaggacgccgtgcaggg

SEQ ID NO: 1937 - DNA, 20 bp, Artificial Sequence
  Synthetic
catagaggacgccgtgcagg

SEQ ID NO: 1938 - DNA, 20 bp, Artificial Sequence
  Synthetic
catagaggacgccgtgcagg

SEQ ID NO: 1939 - DNA, 20 bp, Artificial Sequence
  Synthetic
catagaggacgccgtgcagg

SEQ ID NO: 1940 - DNA, 20 bp, Artificial Sequence
  Synthetic
acatagaggacgccgtgcag

SEQ ID NO: 1941 - DNA, 20 bp, Artificial Sequence
  Synthetic
acatagaggacgccgtgcag

SEQ ID NO: 1942 - DNA, 20 bp, Artificial Sequence
  Synthetic
acatagaggacgccgtgcag

SEQ ID NO: 1943 - DNA, 20 bp, Artificial Sequence
  Synthetic
cacatagaggacgccgtgca

SEQ ID NO: 1944 - DNA, 20 bp, Artificial Sequence
  Synthetic
cacatagaggacgccgtgca

SEQ ID NO: 1945 - DNA, 20 bp, Artificial Sequence
  Synthetic
cacatagaggacgccgtgca

SEQ ID NO: 1946 - DNA, 20 bp, Artificial Sequence
  Synthetic
gcacatagaggacgccgtgc

SEQ ID NO: 1947 - DNA, 20 bp, Artificial Sequence
  Synthetic
gcacatagaggacgccgtgc

SEQ ID NO: 1948 - DNA, 20 bp, Artificial Sequence
  Synthetic
gcacatagaggacgccgtgc

SEQ ID NO: 1949 - DNA, 20 bp, Artificial Sequence
  Synthetic
agaggacgccgtgcagggct

SEQ ID NO: 1950 - DNA, 20 bp, Artificial Sequence
  Synthetic
agaggacgccgtgcagggct

SEQ ID NO: 1951 - DNA, 20 bp, Artificial Sequence
  Synthetic
agaggacgccgtgcagggct

SEQ ID NO: 1952 - DNA, 20 bp, Artificial Sequence
  Synthetic
tagaggacgccgtgcagggc

SEQ ID NO: 1953 - DNA, 20 bp, Artificial Sequence
  Synthetic
tagaggacgccgtgcagggc

SEQ ID NO: 1954 - DNA, 20 bp, Artificial Sequence
  Synthetic
tagaggacgccgtgcagggc

SEQ ID NO: 1955 - DNA, 20 bp, Artificial Sequence
  Synthetic
atagaggacgccgtgcaggg

SEQ ID NO: 1956 - DNA, 20 bp, Artificial Sequence
  Synthetic
atagaggacgccgtgcaggg

SEQ ID NO: 1957 - DNA, 20 bp, Artificial Sequence
  Synthetic
atagaggacgccgtgcaggg

SEQ ID NO: 1958 - DNA, 20 bp, Artificial Sequence
  Synthetic
catagaggacgccgtgcagg

SEQ ID NO: 1959 - DNA, 20 bp, Artificial Sequence
  Synthetic
catagaggacgccgtgcagg

SEQ ID NO: 1960 - DNA, 20 bp, Artificial Sequence
  Synthetic
catagaggacgccgtgcagg

SEQ ID NO: 1961 - DNA, 20 bp, Artificial Sequence
  Synthetic
acatagaggacgccgtgcag

SEQ ID NO: 1962 - DNA, 20 bp, Artificial Sequence
  Synthetic
acatagaggacgccgtgcag

SEQ ID NO: 1963 - DNA, 20 bp, Artificial Sequence
  Synthetic
acatagaggacgccgtgcag

SEQ ID NO: 1964 - DNA, 20 bp, Artificial Sequence
  Synthetic
cacatagaggacgccgtgca

SEQ ID NO: 1965 - DNA, 20 bp, Artificial Sequence
  Synthetic
cacatagaggacgccgtgca

SEQ ID NO: 1966 - DNA, 20 bp, Artificial Sequence
  Synthetic
cacatagaggacgccgtgca

SEQ ID NO: 1967 - DNA, 20 bp, Artificial Sequence
  Synthetic
gcacatagaggacgccgtgc

SEQ ID NO: 1968 - DNA, 20 bp, Artificial Sequence
  Synthetic
gcacatagaggacgccgtgc

SEQ ID NO: 1969 - DNA, 20 bp, Artificial Sequence
  Synthetic
gcacatagaggacgccgtgc

SEQ ID NO: 1970 - DNA, 20 bp, Artificial Sequence
  Synthetic
agaggacgccgtgcagggcu

SEQ ID NO: 1971 - DNA, 20 bp, Artificial Sequence
  Synthetic
agaggacgccgtgcagggcu

SEQ ID NO: 1972 - DNA, 20 bp, Artificial Sequence
  Synthetic
agaggacgccgtgcagggcu

SEQ ID NO: 1973 - DNA, 20 bp, Artificial Sequence
  Synthetic
tagaggacgccgtgcagggc

SEQ ID NO: 1974 - DNA, 20 bp, Artificial Sequence
  Synthetic
tagaggacgccgtgcagggc

SEQ ID NO: 1975 - DNA, 20 bp, Artificial Sequence
  Synthetic
tagaggacgccgtgcagggc

SEQ ID NO: 1976 - DNA, 20 bp, Artificial Sequence
  Synthetic
atagaggacgccgtgcaggg

SEQ ID NO: 1977 - DNA, 20 bp, Artificial Sequence
  Synthetic
atagaggacgccgtgcaggg

SEQ ID NO: 1978 - DNA, 20 bp, Artificial Sequence
  Synthetic
atagaggacgccgtgcaggg

SEQ ID NO: 1979 - DNA, 20 bp, Artificial Sequence
  Synthetic
catagaggacgccgtgcagg

SEQ ID NO: 1980 - DNA, 20 bp, Artificial Sequence
  Synthetic
catagaggacgccgtgcagg

SEQ ID NO: 1981 - DNA, 20 bp, Artificial Sequence
  Synthetic
catagaggacgccgtgcagg

SEQ ID NO: 1982 - DNA, 20 bp, Artificial Sequence
  Synthetic
acatagaggacgccgugcag

SEQ ID NO: 1983 - DNA, 20 bp, Artificial Sequence
  Synthetic
acatagaggacgccgugcag

SEQ ID NO: 1984 - DNA, 20 bp, Artificial Sequence
  Synthetic
acatagaggacgccgugcag

SEQ ID NO: 1985 - DNA, 20 bp, Artificial Sequence
  Synthetic
cacatagaggacgccgugca

SEQ ID NO: 1986 - DNA, 20 bp, Artificial Sequence
  Synthetic
cacatagaggacgccgugca

SEQ ID NO: 1987 - DNA, 20 bp, Artificial Sequence
  Synthetic
cacatagaggacgccgugca

SEQ ID NO: 1988 - DNA, 20 bp, Artificial Sequence
  Synthetic
gcacatagaggacgccgugc

SEQ ID NO: 1989 - DNA, 20 bp, Artificial Sequence
  Synthetic
gcacatagaggacgccgugc

SEQ ID NO: 1990 - DNA, 20 bp, Artificial Sequence
  Synthetic
gcacatagaggacgccgugc

SEQ ID NO: 1991 - DNA, 20 bp, Artificial Sequence
  Synthetic
agaggacgccgtgcagggct

SEQ ID NO: 1992 - DNA, 20 bp, Artificial Sequence
  Synthetic
agaggacgccgtgcagggct

SEQ ID NO: 1993 - DNA, 20 bp, Artificial Sequence
  Synthetic
agaggacgccgtgcagggct

SEQ ID NO: 1994 - DNA, 20 bp, Artificial Sequence
  Synthetic
uagaggacgccgtgcagggc

SEQ ID NO: 1995 - DNA, 20 bp, Artificial Sequence
  Synthetic
uagaggacgccgtgcagggc

SEQ ID NO: 1996 - DNA, 20 bp, Artificial Sequence
  Synthetic
uagaggacgccgtgcagggc

SEQ ID NO: 1997 - DNA, 20 bp, Artificial Sequence
  Synthetic
auagaggacgccgtgcaggg

SEQ ID NO: 1998 - DNA, 20 bp, Artificial Sequence
  Synthetic
auagaggacgccgtgcaggg

SEQ ID NO: 1999 - DNA, 20 bp, Artificial Sequence
  Synthetic
auagaggacgccgtgcaggg

SEQ ID NO: 2000 - DNA, 20 bp, Artificial Sequence
  Synthetic
cauagaggacgccgtgcagg

SEQ ID NO: 2001 - DNA, 20 bp, Artificial Sequence
  Synthetic
cauagaggacgccgtgcagg

SEQ ID NO: 2002 - DNA, 20 bp, Artificial Sequence
  Synthetic
cauagaggacgccgtgcagg

SEQ ID NO: 2003 - DNA, 20 bp, Artificial Sequence
  Synthetic
acauagaggacgccgtgcag

SEQ ID NO: 2004 - DNA, 20 bp, Artificial Sequence
  Synthetic
acauagaggacgccgtgcag

SEQ ID NO: 2005 - DNA, 20 bp, Artificial Sequence
  Synthetic
acauagaggacgccgtgcag

SEQ ID NO: 2006 - DNA, 20 bp, Artificial Sequence
  Synthetic
cacauagaggacgccgtgca

SEQ ID NO: 2007 - DNA, 20 bp, Artificial Sequence
  Synthetic
cacauagaggacgccgtgca

SEQ ID NO: 2008 - DNA, 20 bp, Artificial Sequence
  Synthetic
cacauagaggacgccgtgca

SEQ ID NO: 2009 - DNA, 20 bp, Artificial Sequence
  Synthetic
gcacatagaggacgccgtgc

SEQ ID NO: 2010 - DNA, 20 bp, Artificial Sequence
  Synthetic
gcacatagaggacgccgtgc

SEQ ID NO: 2011 - DNA, 20 bp, Artificial Sequence
  Synthetic
gcacatagaggacgccgtgc

SEQ ID NO: 2012 - DNA, 20 bp, Artificial Sequence
  Synthetic
tgagcggagaaaccctccaa

SEQ ID NO: 2013 - DNA, 20 bp, Artificial Sequence
  Synthetic
tgagcggagaaaccctccaa

SEQ ID NO: 2014 - DNA, 20 bp, Artificial Sequence
  Synthetic
tgagcggagaaaccctccaa

SEQ ID NO: 2015 - DNA, 20 bp, Artificial Sequence
  Synthetic
ctgagcggagaaaccctcca

SEQ ID NO: 2016 - DNA, 20 bp, Artificial Sequence
  Synthetic
ctgagcggagaaaccctcca

SEQ ID NO: 2017 - DNA, 20 bp, Artificial Sequence
  Synthetic
ctgagcggagaaaccctcca

SEQ ID NO: 2018 - DNA, 20 bp, Artificial Sequence
  Synthetic
gctgagcggagaaaccctcc

SEQ ID NO: 2019 - DNA, 20 bp, Artificial Sequence
  Synthetic
gctgagcggagaaaccctcc

SEQ ID NO: 2020 - DNA, 20 bp, Artificial Sequence
  Synthetic
gctgagcggagaaaccctcc

SEQ ID NO: 2021 - DNA, 20 bp, Artificial Sequence
  Synthetic
ggctgagcggagaaaccctc

SEQ ID NO: 2022 - DNA, 20 bp, Artificial Sequence
  Synthetic
ggctgagcggagaaaccctc

SEQ ID NO: 2023 - DNA, 20 bp, Artificial Sequence
  Synthetic
ggctgagcggagaaaccctc

SEQ ID NO: 2024 - DNA, 20 bp, Artificial Sequence
  Synthetic
aggctgagcggagaaaccct

SEQ ID NO: 2025 - DNA, 20 bp, Artificial Sequence
  Synthetic
aggctgagcggagaaaccct

SEQ ID NO: 2026 - DNA, 20 bp, Artificial Sequence
  Synthetic
aggctgagcggagaaaccct

SEQ ID NO: 2027 - DNA, 20 bp, Artificial Sequence
  Synthetic
aaggctgagcggagaaaccc

SEQ ID NO: 2028 - DNA, 20 bp, Artificial Sequence
  Synthetic
aaggctgagcggagaaaccc

SEQ ID NO: 2029 - DNA, 20 bp, Artificial Sequence
  Synthetic
aaggctgagcggagaaaccc

SEQ ID NO: 2030 - DNA, 20 bp, Artificial Sequence
  Synthetic
caaggctgagcggagaaacc

SEQ ID NO: 2031 - DNA, 20 bp, Artificial Sequence
  Synthetic
caaggctgagcggagaaacc

SEQ ID NO: 2032 - DNA, 20 bp, Artificial Sequence
  Synthetic
caaggctgagcggagaaacc

SEQ ID NO: 2033 - DNA, 20 bp, Artificial Sequence
  Synthetic
tgagcggagaaacccuccaa

SEQ ID NO: 2034 - DNA, 20 bp, Artificial Sequence
  Synthetic
tgagcggagaaacccuccaa

SEQ ID NO: 2035 - DNA, 20 bp, Artificial Sequence
  Synthetic
tgagcggagaaacccuccaa

SEQ ID NO: 2036 - DNA, 20 bp, Artificial Sequence
  Synthetic
ctgagcggagaaacccucca

SEQ ID NO: 2037 - DNA, 20 bp, Artificial Sequence
  Synthetic
ctgagcggagaaacccucca

SEQ ID NO: 2038 - DNA, 20 bp, Artificial Sequence
  Synthetic
ctgagcggagaaacccucca

SEQ ID NO: 2039 - DNA, 20 bp, Artificial Sequence
  Synthetic
gctgagcggagaaacccucc

SEQ ID NO: 2040 - DNA, 20 bp, Artificial Sequence
  Synthetic
gctgagcggagaaacccucc

SEQ ID NO: 2041 - DNA, 20 bp, Artificial Sequence
  Synthetic
gctgagcggagaaacccucc

SEQ ID NO: 2042 - DNA, 20 bp, Artificial Sequence
  Synthetic
ggctgagcggagaaacccuc

SEQ ID NO: 2043 - DNA, 20 bp, Artificial Sequence
  Synthetic
ggctgagcggagaaacccuc

SEQ ID NO: 2044 - DNA, 20 bp, Artificial Sequence
  Synthetic
ggctgagcggagaaacccuc

SEQ ID NO: 2045 - DNA, 20 bp, Artificial Sequence
  Synthetic
aggctgagcggagaaacccu

SEQ ID NO: 2046 - DNA, 20 bp, Artificial Sequence
  Synthetic
aggctgagcggagaaacccu

SEQ ID NO: 2047 - DNA, 20 bp, Artificial Sequence
  Synthetic
aggctgagcggagaaacccu

SEQ ID NO: 2048 - DNA, 20 bp, Artificial Sequence
  Synthetic
aaggctgagcggagaaaccc

SEQ ID NO: 2049 - DNA, 20 bp, Artificial Sequence
  Synthetic
aaggctgagcggagaaaccc

SEQ ID NO: 2050 - DNA, 20 bp, Artificial Sequence
  Synthetic
aaggctgagcggagaaaccc

SEQ ID NO: 2051 - DNA, 20 bp, Artificial Sequence
  Synthetic
caaggctgagcggagaaacc

SEQ ID NO: 2052 - DNA, 20 bp, Artificial Sequence
  Synthetic
caaggctgagcggagaaacc

SEQ ID NO: 2053 - DNA, 20 bp, Artificial Sequence
  Synthetic
caaggctgagcggagaaacc

SEQ ID NO: 2054 - DNA, 20 bp, Artificial Sequence
  Synthetic
ugagcggagaaaccctccaa

SEQ ID NO: 2055 - DNA, 20 bp, Artificial Sequence
  Synthetic
ugagcggagaaaccctccaa

SEQ ID NO: 2056 - DNA, 20 bp, Artificial Sequence
  Synthetic
ugagcggagaaaccctccaa

SEQ ID NO: 2057 - DNA, 20 bp, Artificial Sequence
  Synthetic
cugagcggagaaaccctcca

SEQ ID NO: 2058 - DNA, 20 bp, Artificial Sequence
  Synthetic
cugagcggagaaaccctcca

SEQ ID NO: 2059 - DNA, 20 bp, Artificial Sequence
  Synthetic
cugagcggagaaaccctcca

SEQ ID NO: 2060 - DNA, 20 bp, Artificial Sequence
  Synthetic
gcugagcggagaaaccctcc

SEQ ID NO: 2061 - DNA, 20 bp, Artificial Sequence
  Synthetic
gcugagcggagaaaccctcc

SEQ ID NO: 2062 - DNA, 20 bp, Artificial Sequence
  Synthetic
gcugagcggagaaaccctcc

SEQ ID NO: 2063 - DNA, 20 bp, Artificial Sequence
  Synthetic
ggcugagcggagaaaccctc

SEQ ID NO: 2064 - DNA, 20 bp, Artificial Sequence
  Synthetic
ggcugagcggagaaaccctc

SEQ ID NO: 2065 - DNA, 20 bp, Artificial Sequence
  Synthetic
ggcugagcggagaaaccctc

SEQ ID NO: 2066 - DNA, 20 bp, Artificial Sequence
  Synthetic
aggcugagcggagaaaccct

SEQ ID NO: 2067 - DNA, 20 bp, Artificial Sequence
  Synthetic
aggcugagcggagaaaccct

SEQ ID NO: 2068 - DNA, 20 bp, Artificial Sequence
  Synthetic
aggcugagcggagaaaccct

SEQ ID NO: 2069 - DNA, 20 bp, Artificial Sequence
  Synthetic
aaggctgagcggagaaaccc

SEQ ID NO: 2070 - DNA, 20 bp, Artificial Sequence
  Synthetic
aaggctgagcggagaaaccc

SEQ ID NO: 2071 - DNA, 20 bp, Artificial Sequence
  Synthetic
aaggctgagcggagaaaccc

SEQ ID NO: 2072 - DNA, 20 bp, Artificial Sequence
  Synthetic
caaggctgagcggagaaacc

SEQ ID NO: 2073 - DNA, 20 bp, Artificial Sequence
  Synthetic
caaggctgagcggagaaacc

SEQ ID NO: 2074 - DNA, 20 bp, Artificial Sequence
  Synthetic
caaggctgagcggagaaacc

SEQ ID NO: 2075 - DNA, 20 bp, Artificial Sequence
  Synthetic
tgagcggagaaaccctccaa

SEQ ID NO: 2076 - DNA, 20 bp, Artificial Sequence
  Synthetic
tgagcggagaaaccctccaa

SEQ ID NO: 2077 - DNA, 20 bp, Artificial Sequence
  Synthetic
tgagcggagaaaccctccaa

SEQ ID NO: 2078 - DNA, 20 bp, Artificial Sequence
  Synthetic
ctgagcggagaaaccctcca

SEQ ID NO: 2079 - DNA, 20 bp, Artificial Sequence
  Synthetic
ctgagcggagaaaccctcca

SEQ ID NO: 2080 - DNA, 20 bp, Artificial Sequence
  Synthetic
ctgagcggagaaaccctcca

SEQ ID NO: 2081 - DNA, 20 bp, Artificial Sequence
  Synthetic
gctgagcggagaaaccctcc

SEQ ID NO: 2082 - DNA, 20 bp, Artificial Sequence
  Synthetic
gctgagcggagaaaccctcc

SEQ ID NO: 2083 - DNA, 20 bp, Artificial Sequence
  Synthetic
gctgagcggagaaaccctcc

SEQ ID NO: 2084 - DNA, 20 bp, Artificial Sequence
  Synthetic
ggctgagcggagaaaccctc

SEQ ID NO: 2085 - DNA, 20 bp, Artificial Sequence
  Synthetic
ggctgagcggagaaaccctc

SEQ ID NO: 2086 - DNA, 20 bp, Artificial Sequence
  Synthetic
ggctgagcggagaaaccctc

SEQ ID NO: 2087 - DNA, 20 bp, Artificial Sequence
  Synthetic
aggctgagcggagaaaccct

SEQ ID NO: 2088 - DNA, 20 bp, Artificial Sequence
  Synthetic
aggctgagcggagaaaccct

SEQ ID NO: 2089 - DNA, 20 bp, Artificial Sequence
  Synthetic
aggctgagcggagaaaccct

SEQ ID NO: 2090 - DNA, 20 bp, Artificial Sequence
  Synthetic
aaggctgagcggagaaaccc

SEQ ID NO: 2091 - DNA, 20 bp, Artificial Sequence
  Synthetic
aaggctgagcggagaaaccc

SEQ ID NO: 2092 - DNA, 20 bp, Artificial Sequence
  Synthetic
aaggctgagcggagaaaccc

SEQ ID NO: 2093 - DNA, 20 bp, Artificial Sequence
  Synthetic
caaggctgagcggagaaacc

SEQ ID NO: 2094 - DNA, 20 bp, Artificial Sequence
  Synthetic
caaggctgagcggagaaacc

SEQ ID NO: 2095 - DNA, 20 bp, Artificial Sequence
  Synthetic
caaggctgagcggagaaacc

SEQ ID NO: 2096 - DNA, 20 bp, Artificial Sequence
  Synthetic
tgagcggagaaaccctccaa

SEQ ID NO: 2097 - DNA, 20 bp, Artificial Sequence
  Synthetic
tgagcggagaaaccctccaa

SEQ ID NO: 2098 - DNA, 20 bp, Artificial Sequence
  Synthetic
tgagcggagaaaccctccaa

SEQ ID NO: 2099 - DNA, 20 bp, Artificial Sequence
  Synthetic
ctgagcggagaaaccctcca

SEQ ID NO: 2100 - DNA, 20 bp, Artificial Sequence
  Synthetic
ctgagcggagaaaccctcca

SEQ ID NO: 2101 - DNA, 20 bp, Artificial Sequence
  Synthetic
ctgagcggagaaaccctcca

SEQ ID NO: 2102 - DNA, 20 bp, Artificial Sequence
  Synthetic
gctgagcggagaaaccctcc

SEQ ID NO: 2103 - DNA, 20 bp, Artificial Sequence
  Synthetic
gctgagcggagaaaccctcc

SEQ ID NO: 2104 - DNA, 20 bp, Artificial Sequence
  Synthetic
gctgagcggagaaaccctcc

SEQ ID NO: 2105 - DNA, 20 bp, Artificial Sequence
  Synthetic
ggctgagcggagaaaccctc

SEQ ID NO: 2106 - DNA, 20 bp, Artificial Sequence
  Synthetic
ggctgagcggagaaaccctc

SEQ ID NO: 2107 - DNA, 20 bp, Artificial Sequence
  Synthetic
ggctgagcggagaaaccctc

SEQ ID NO: 2108 - DNA, 20 bp, Artificial Sequence
  Synthetic
aggctgagcggagaaaccct

SEQ ID NO: 2109 - DNA, 20 bp, Artificial Sequence
  Synthetic
aggctgagcggagaaaccct

SEQ ID NO: 2110 - DNA, 20 bp, Artificial Sequence
  Synthetic
aggctgagcggagaaaccct

SEQ ID NO: 2111 - DNA, 20 bp, Artificial Sequence
  Synthetic
aaggctgagcggagaaaccc

SEQ ID NO: 2112 - DNA, 20 bp, Artificial Sequence
  Synthetic
aaggctgagcggagaaaccc

SEQ ID NO: 2113 - DNA, 20 bp, Artificial Sequence
  Synthetic
aaggctgagcggagaaaccc

SEQ ID NO: 2114 - DNA, 20 bp, Artificial Sequence
  Synthetic
caaggctgagcggagaaacc

SEQ ID NO: 2115 - DNA, 20 bp, Artificial Sequence
  Synthetic
caaggctgagcggagaaacc

SEQ ID NO: 2116 - DNA, 20 bp, Artificial Sequence
  Synthetic
caaggctgagcggagaaacc

SEQ ID NO: 2117 - DNA, 20 bp, Artificial Sequence
  Synthetic
tgagcggagaaacccuccaa

SEQ ID NO: 2118 - DNA, 20 bp, Artificial Sequence
  Synthetic
tgagcggagaaacccuccaa

SEQ ID NO: 2119 - DNA, 20 bp, Artificial Sequence
  Synthetic
tgagcggagaaacccuccaa

SEQ ID NO: 2120 - DNA, 20 bp, Artificial Sequence
  Synthetic
ctgagcggagaaacccucca

SEQ ID NO: 2121 - DNA, 20 bp, Artificial Sequence
  Synthetic
ctgagcggagaaacccucca

SEQ ID NO: 2122 - DNA, 20 bp, Artificial Sequence
  Synthetic
ctgagcggagaaacccucca

SEQ ID NO: 2123 - DNA, 20 bp, Artificial Sequence
  Synthetic
gctgagcggagaaacccucc

SEQ ID NO: 2124 - DNA, 20 bp, Artificial Sequence
  Synthetic
gctgagcggagaaacccucc

SEQ ID NO: 2125 - DNA, 20 bp, Artificial Sequence
  Synthetic
gctgagcggagaaacccucc

SEQ ID NO: 2126 - DNA, 20 bp, Artificial Sequence
  Synthetic
ggctgagcggagaaacccuc

SEQ ID NO: 2127 - DNA, 20 bp, Artificial Sequence
  Synthetic
ggctgagcggagaaacccuc

SEQ ID NO: 2128 - DNA, 20 bp, Artificial Sequence
  Synthetic
ggctgagcggagaaacccuc

SEQ ID NO: 2129 - DNA, 20 bp, Artificial Sequence
  Synthetic
aggctgagcggagaaacccu

SEQ ID NO: 2130 - DNA, 20 bp, Artificial Sequence
  Synthetic
aggctgagcggagaaacccu

SEQ ID NO: 2131 - DNA, 20 bp, Artificial Sequence
  Synthetic
aggctgagcggagaaacccu

SEQ ID NO: 2132 - DNA, 20 bp, Artificial Sequence
  Synthetic
aaggctgagcggagaaaccc

SEQ ID NO: 2133 - DNA, 20 bp, Artificial Sequence
  Synthetic
aaggctgagcggagaaaccc

SEQ ID NO: 2134 - DNA, 20 bp, Artificial Sequence
  Synthetic
aaggctgagcggagaaaccc

SEQ ID NO: 2135 - DNA, 20 bp, Artificial Sequence
  Synthetic
caaggctgagcggagaaacc

SEQ ID NO: 2136 - DNA, 20 bp, Artificial Sequence
  Synthetic
caaggctgagcggagaaacc

SEQ ID NO: 2137 - DNA, 20 bp, Artificial Sequence
  Synthetic
caaggctgagcggagaaacc

SEQ ID NO: 2138 - DNA, 20 bp, Artificial Sequence
  Synthetic
ugagcggagaaaccctccaa

SEQ ID NO: 2139 - DNA, 20 bp, Artificial Sequence
  Synthetic
ugagcggagaaaccctccaa

SEQ ID NO: 2140 - DNA, 20 bp, Artificial Sequence
  Synthetic
ugagcggagaaaccctccaa

SEQ ID NO: 2141 - DNA, 20 bp, Artificial Sequence
  Synthetic
cugagcggagaaaccctcca

SEQ ID NO: 2142 - DNA, 20 bp, Artificial Sequence
  Synthetic
cugagcggagaaaccctcca

SEQ ID NO: 2143 - DNA, 20 bp, Artificial Sequence
  Synthetic
cugagcggagaaaccctcca

SEQ ID NO: 2144 - DNA, 20 bp, Artificial Sequence
  Synthetic
gcugagcggagaaaccctcc

SEQ ID NO: 2145 - DNA, 20 bp, Artificial Sequence
  Synthetic
gcugagcggagaaaccctcc

SEQ ID NO: 2146 - DNA, 20 bp, Artificial Sequence
  Synthetic
gcugagcggagaaaccctcc

SEQ ID NO: 2147 - DNA, 20 bp, Artificial Sequence
  Synthetic
ggcugagcggagaaaccctc

SEQ ID NO: 2148 - DNA, 20 bp, Artificial Sequence
  Synthetic
ggcugagcggagaaaccctc

SEQ ID NO: 2149 - DNA, 20 bp, Artificial Sequence
  Synthetic
ggcugagcggagaaaccctc

SEQ ID NO: 2150 - DNA, 20 bp, Artificial Sequence
  Synthetic
aggcugagcggagaaaccct

SEQ ID NO: 2151 - DNA, 20 bp, Artificial Sequence
  Synthetic
aggcugagcggagaaaccct

SEQ ID NO: 2152 - DNA, 20 bp, Artificial Sequence
  Synthetic
aggcugagcggagaaaccct

SEQ ID NO: 2153 - DNA, 20 bp, Artificial Sequence
  Synthetic
aaggctgagcggagaaaccc

SEQ ID NO: 2154 - DNA, 20 bp, Artificial Sequence
  Synthetic
aaggctgagcggagaaaccc

SEQ ID NO: 2155 - DNA, 20 bp, Artificial Sequence
  Synthetic
aaggctgagcggagaaaccc

SEQ ID NO: 2156 - DNA, 20 bp, Artificial Sequence
  Synthetic
caaggctgagcggagaaacc

SEQ ID NO: 2157 - DNA, 20 bp, Artificial Sequence
  Synthetic
caaggctgagcggagaaacc

SEQ ID NO: 2158 - DNA, 20 bp, Artificial Sequence
  Synthetic
caaggctgagcggagaaacc

SEQ ID NO: 2159 - DNA, 20 bp, Artificial Sequence
  Synthetic
guugatctgtagcagcagct

SEQ ID NO: 2160 - DNA, 20 bp, Artificial Sequence
  Synthetic
guugatctgtagcagcagct

SEQ ID NO: 2161 - DNA, 20 bp, Artificial Sequence
  Synthetic
guugatctgtagcagcagct

SEQ ID NO: 2162 - DNA, 20 bp, Artificial Sequence
  Synthetic
guugatctgtagcagcagct

SEQ ID NO: 2163 - DNA, 20 bp, Artificial Sequence
  Synthetic
gtgcacacagtagatgaggg

SEQ ID NO: 2164 - DNA, 20 bp, Artificial Sequence
  Synthetic
gtgcacacagtagatgaggg

SEQ ID NO: 2165 - DNA, 20 bp, Artificial Sequence
  Synthetic
gugcacacagtagatgaggg

SEQ ID NO: 2166 - DNA, 20 bp, Artificial Sequence
  Synthetic
cacagtagatgagggagcag

SEQ ID NO: 2167 - DNA, 20 bp, Artificial Sequence
  Synthetic
cacagtagatgagggagcag

SEQ ID NO: 2168 - DNA, 20 bp, Artificial Sequence
  Synthetic
cacagtagatgagggagcag

SEQ ID NO: 2169 - DNA, 20 bp, Artificial Sequence
  Synthetic
acacagtagatgagggagca

SEQ ID NO: 2170 - DNA, 20 bp, Artificial Sequence
  Synthetic
acacagtagatgagggagca

SEQ ID NO: 2171 - DNA, 20 bp, Artificial Sequence
  Synthetic
acacagtagatgagggagca

SEQ ID NO: 2172 - DNA, 20 bp, Artificial Sequence
  Synthetic
cacacagtagatgagggagc

SEQ ID NO: 2173 - DNA, 20 bp, Artificial Sequence
  Synthetic
cacacagtagatgagggagc

SEQ ID NO: 2174 - DNA, 20 bp, Artificial Sequence
  Synthetic
cacacagtagatgagggagc

SEQ ID NO: 2175 - DNA, 20 bp, Artificial Sequence
  Synthetic
gcacacagtagatgagggag

SEQ ID NO: 2176 - DNA, 20 bp, Artificial Sequence
  Synthetic
gcacacagtagatgagggag

SEQ ID NO: 2177 - DNA, 20 bp, Artificial Sequence
  Synthetic
gcacacagtagatgagggag

SEQ ID NO: 2178 - DNA, 20 bp, Artificial Sequence
  Synthetic
tgcacacagtagatgaggga

SEQ ID NO: 2179 - DNA, 20 bp, Artificial Sequence
  Synthetic
tgcacacagtagatgaggga

SEQ ID NO: 2180 - DNA, 20 bp, Artificial Sequence
  Synthetic
tgcacacagtagatgaggga

SEQ ID NO: 2181 - DNA, 20 bp, Artificial Sequence
  Synthetic
gtgcacacagtagatgaggg

SEQ ID NO: 2182 - DNA, 20 bp, Artificial Sequence
  Synthetic
gtgcacacagtagatgaggg

SEQ ID NO: 2183 - DNA, 20 bp, Artificial Sequence
  Synthetic
agtgcacacagtagatgagg

SEQ ID NO: 2184 - DNA, 20 bp, Artificial Sequence
  Synthetic
agtgcacacagtagatgagg

SEQ ID NO: 2185 - DNA, 20 bp, Artificial Sequence
  Synthetic
agtgcacacagtagatgagg

SEQ ID NO: 2186 - DNA, 20 bp, Artificial Sequence
  Synthetic
cacagtagatgagggagcag

SEQ ID NO: 2187 - DNA, 20 bp, Artificial Sequence
  Synthetic
cacagtagatgagggagcag

SEQ ID NO: 2188 - DNA, 20 bp, Artificial Sequence
  Synthetic
cacagtagatgagggagcag

SEQ ID NO: 2189 - DNA, 20 bp, Artificial Sequence
  Synthetic
acacagtagatgagggagca

SEQ ID NO: 2190 - DNA, 20 bp, Artificial Sequence
  Synthetic
acacagtagatgagggagca

SEQ ID NO: 2191 - DNA, 20 bp, Artificial Sequence
  Synthetic
acacagtagatgagggagca

SEQ ID NO: 2192 - DNA, 20 bp, Artificial Sequence
  Synthetic
cacacagtagatgagggagc

SEQ ID NO: 2193 - DNA, 20 bp, Artificial Sequence
  Synthetic
cacacagtagatgagggagc

SEQ ID NO: 2194 - DNA, 20 bp, Artificial Sequence
  Synthetic
cacacagtagatgagggagc

SEQ ID NO: 2195 - DNA, 20 bp, Artificial Sequence
  Synthetic
gcacacagtagatgagggag

SEQ ID NO: 2196 - DNA, 20 bp, Artificial Sequence
  Synthetic
gcacacagtagatgagggag

SEQ ID NO: 2197 - DNA, 20 bp, Artificial Sequence
  Synthetic
gcacacagtagatgagggag

SEQ ID NO: 2198 - DNA, 20 bp, Artificial Sequence
  Synthetic
tgcacacagtagatgaggga

SEQ ID NO: 2199 - DNA, 20 bp, Artificial Sequence
  Synthetic
tgcacacagtagatgaggga

SEQ ID NO: 2200 - DNA, 20 bp, Artificial Sequence
  Synthetic
tgcacacagtagatgaggga

SEQ ID NO: 2201 - DNA, 20 bp, Artificial Sequence
  Synthetic
gtgcacacagtagatgaggg

SEQ ID NO: 2202 - DNA, 20 bp, Artificial Sequence
  Synthetic
gtgcacacagtagatgaggg

SEQ ID NO: 2203 - DNA, 20 bp, Artificial Sequence
  Synthetic
agtgcacacagtagaugagg

SEQ ID NO: 2204 - DNA, 20 bp, Artificial Sequence
  Synthetic
agtgcacacagtagaugagg

SEQ ID NO: 2205 - DNA, 20 bp, Artificial Sequence
  Synthetic
agtgcacacagtagaugagg

SEQ ID NO: 2206 - DNA, 20 bp, Artificial Sequence
  Synthetic
gcacacagtagatgagggag

SEQ ID NO: 2207 - DNA, 20 bp, Artificial Sequence
  Synthetic
gcacacagtagatgagggag

SEQ ID NO: 2208 - DNA, 20 bp, Artificial Sequence
  Synthetic
gcacacagtagatgagggag

SEQ ID NO: 2209 - DNA, 20 bp, Artificial Sequence
  Synthetic
ugcacacagtagatgaggga

SEQ ID NO: 2210 - DNA, 20 bp, Artificial Sequence
  Synthetic
ugcacacagtagatgaggga

SEQ ID NO: 2211 - DNA, 20 bp, Artificial Sequence
  Synthetic
ugcacacagtagatgaggga

SEQ ID NO: 2212 - DNA, 20 bp, Artificial Sequence
  Synthetic
gugcacacagtagatgaggg

SEQ ID NO: 2213 - DNA, 20 bp, Artificial Sequence
  Synthetic
gugcacacagtagatgaggg

SEQ ID NO: 2214 - DNA, 20 bp, Artificial Sequence
  Synthetic
agugcacacagtagatgagg

SEQ ID NO: 2215 - DNA, 20 bp, Artificial Sequence
  Synthetic
agugcacacagtagatgagg

SEQ ID NO: 2216 - DNA, 20 bp, Artificial Sequence
  Synthetic
agugcacacagtagatgagg

SEQ ID NO: 2217 - DNA, 20 bp, Artificial Sequence
  Synthetic
gugcacacagtagatgaggg

SEQ ID NO: 2218 - DNA, 20 bp, Artificial Sequence
  Synthetic
gugcacacagtagatgaggg

SEQ ID NO: 2219 - DNA, 20 bp, Artificial Sequence
  Synthetic
gugcacacagtagatgaggg

SEQ ID NO: 2220 - DNA, 20 bp, Artificial Sequence
  Synthetic
gugcacacagtagatgaggg

SEQ ID NO: 2221 - DNA, 20 bp, Artificial Sequence
  Synthetic
gtgcacacagtagatgaggg

SEQ ID NO: 2222 - RNA, 20 nt, Artificial Sequence
  Synthetic
agcugcugcuacagaucaac

SEQ ID NO: 2223 - RNA, 20 nt, Artificial Sequence
  Synthetic
agcugcugcugcagaucaac

SEQ ID NO: 2224 - DNA, 20 bp, Artificial Sequence
  Synthetic
guugatctgtagcagcagct

SEQ ID NO: 2225 - DNA, 20 bp, Artificial Sequence
  Synthetic
guugatctgtagcagcagct

SEQ ID NO: 2226 - DNA, 20 bp, Artificial Sequence
  Synthetic
guugatctgtagcagcagct

SEQ ID NO: 2227 - DNA, 20 bp, Artificial Sequence
  Synthetic
guugatctgtagcagcagct

SEQ ID NO: 2228 - DNA, 20 bp, Artificial Sequence
  Synthetic
guugatctgtagcagcagct

SEQ ID NO: 2229 - DNA, 20 bp, Artificial Sequence
  Synthetic
guugatctgtagcagcagct

SEQ ID NO: 2230 - DNA, 20 bp, Artificial Sequence
  Synthetic
guugatctgtagcagcagct

SEQ ID NO: 2231 - DNA, 20 bp, Artificial Sequence
  Synthetic
guugatctgtagcagcagct

SEQ ID NO: 2232 - DNA, 20 bp, Artificial Sequence
  Synthetic
guugatctgtagcagcagct

SEQ ID NO: 2233 - DNA, 20 bp, Artificial Sequence
  Synthetic
guugatctgtagcagcagct

SEQ ID NO: 2234 - DNA, 20 bp, Artificial Sequence
  Synthetic
guugatctgtagcagcagct

SEQ ID NO: 2235 - DNA, 20 bp, Artificial Sequence
  Synthetic
guugatctgtagcagcagct

SEQ ID NO: 2236 - DNA, 20 bp, Artificial Sequence
  Synthetic
guugatctgtagcagcagct

SEQ ID NO: 2237 - DNA, 20 bp, Artificial Sequence
  Synthetic
guugatctgtagcagcagct

SEQ ID NO: 2238 - DNA, 20 bp, Artificial Sequence
  Synthetic
guugatctgtagcagcagct

SEQ ID NO: 2239 - DNA, 20 bp, Artificial Sequence
  Synthetic
guugatctgtagcagcagct

SEQ ID NO: 2240 - DNA, 20 bp, Artificial Sequence
  Synthetic
guugatctgtagcagcagct

SEQ ID NO: 2241 - DNA, 19 bp, Artificial Sequence
  Synthetic
uugatctgtagcagcagct

SEQ ID NO: 2242 - DNA, 21 bp, Artificial Sequence
  Synthetic
gguugatctgtagcagcagct

SEQ ID NO: 2243 - DNA, 20 bp, Artificial Sequence
  Synthetic
guugatctgtagcagcagct

SEQ ID NO: 2244 - DNA, 20 bp, Artificial Sequence
  Synthetic
guugatctgtagcagcagct

SEQ ID NO: 2245 - DNA, 20 bp, Artificial Sequence
  Synthetic
cacagtagatgagggagcag

SEQ ID NO: 2246 - DNA, 20 bp, Artificial Sequence
  Synthetic
cacagtagatgagggagcag

SEQ ID NO: 2247 - DNA, 20 bp, Artificial Sequence
  Synthetic
cacagtagatgagggagcag

SEQ ID NO: 2248 - DNA, 20 bp, Artificial Sequence
  Synthetic
acacagtagatgagggagca

SEQ ID NO: 2249 - DNA, 20 bp, Artificial Sequence
  Synthetic
acacagtagatgagggagca

SEQ ID NO: 2250 - DNA, 20 bp, Artificial Sequence
  Synthetic
acacagtagatgagggagca

SEQ ID NO: 2251 - DNA, 20 bp, Artificial Sequence
  Synthetic
cacacagtagatgagggagc

SEQ ID NO: 2252 - DNA, 20 bp, Artificial Sequence
  Synthetic
cacacagtagatgagggagc

SEQ ID NO: 2253 - DNA, 20 bp, Artificial Sequence
  Synthetic
cacacagtagatgagggagc

SEQ ID NO: 2254 - DNA, 20 bp, Artificial Sequence
  Synthetic
gcacacagtagatgagggag

SEQ ID NO: 2255 - DNA, 20 bp, Artificial Sequence
  Synthetic
gcacacagtagatgagggag

SEQ ID NO: 2256 - DNA, 20 bp, Artificial Sequence
  Synthetic
gcacacagtagatgagggag

SEQ ID NO: 2257 - DNA, 20 bp, Artificial Sequence
  Synthetic
tgcacacagtagatgaggga

SEQ ID NO: 2258 - DNA, 20 bp, Artificial Sequence
  Synthetic
tgcacacagtagatgaggga

SEQ ID NO: 2259 - DNA, 20 bp, Artificial Sequence
  Synthetic
tgcacacagtagatgaggga

SEQ ID NO: 2260 - DNA, 20 bp, Artificial Sequence
  Synthetic
gtgcacacagtagatgaggg

SEQ ID NO: 2261 - DNA, 20 bp, Artificial Sequence
  Synthetic
gtgcacacagtagatgaggg

SEQ ID NO: 2262 - DNA, 20 bp, Artificial Sequence
  Synthetic
gtgcacacagtagatgaggg

SEQ ID NO: 2263 - DNA, 20 bp, Artificial Sequence
  Synthetic
agtgcacacagtagatgagg

SEQ ID NO: 2264 - DNA, 20 bp, Artificial Sequence
  Synthetic
agtgcacacagtagatgagg

SEQ ID NO: 2265 - DNA, 20 bp, Artificial Sequence
  Synthetic
agtgcacacagtagatgagg

SEQ ID NO: 2266 - DNA, 20 bp, Artificial Sequence
  Synthetic
cacagtagatgagggagcag

SEQ ID NO: 2267 - DNA, 20 bp, Artificial Sequence
  Synthetic
cacagtagatgagggagcag

SEQ ID NO: 2268 - DNA, 20 bp, Artificial Sequence
  Synthetic
cacagtagatgagggagcag

SEQ ID NO: 2269 - DNA, 20 bp, Artificial Sequence
  Synthetic
acacagtagatgagggagca

SEQ ID NO: 2270 - DNA, 20 bp, Artificial Sequence
  Synthetic
acacagtagatgagggagca

SEQ ID NO: 2271 - DNA, 20 bp, Artificial Sequence
  Synthetic
acacagtagatgagggagca

SEQ ID NO: 2272 - DNA, 20 bp, Artificial Sequence
  Synthetic
cacacagtagatgagggagc

SEQ ID NO: 2273 - DNA, 20 bp, Artificial Sequence
  Synthetic
cacacagtagatgagggagc

SEQ ID NO: 2274 - DNA, 20 bp, Artificial Sequence
  Synthetic
cacacagtagatgagggagc

SEQ ID NO: 2275 - DNA, 20 bp, Artificial Sequence
  Synthetic
gcacacagtagatgagggag

SEQ ID NO: 2276 - DNA, 20 bp, Artificial Sequence
  Synthetic
gcacacagtagatgagggag

SEQ ID NO: 2277 - DNA, 20 bp, Artificial Sequence
  Synthetic
gcacacagtagatgagggag

SEQ ID NO: 2278 - DNA, 20 bp, Artificial Sequence
  Synthetic
tgcacacagtagatgaggga

SEQ ID NO: 2279 - DNA, 20 bp, Artificial Sequence
  Synthetic
tgcacacagtagatgaggga

SEQ ID NO: 2280 - DNA, 20 bp, Artificial Sequence
  Synthetic
tgcacacagtagatgaggga

SEQ ID NO: 2281 - DNA, 20 bp, Artificial Sequence
  Synthetic
gtgcacacagtagatgaggg

SEQ ID NO: 2282 - DNA, 20 bp, Artificial Sequence
  Synthetic
gtgcacacagtagatgaggg

SEQ ID NO: 2283 - DNA, 20 bp, Artificial Sequence
  Synthetic
gtgcacacagtagatgaggg

SEQ ID NO: 2284 - DNA, 20 bp, Artificial Sequence
  Synthetic
agtgcacacagtagatgagg

SEQ ID NO: 2285 - DNA, 20 bp, Artificial Sequence
  Synthetic
agtgcacacagtagatgagg

SEQ ID NO: 2286 - DNA, 20 bp, Artificial Sequence
  Synthetic
agtgcacacagtagatgagg

SEQ ID NO: 2287 - DNA, 20 bp, Artificial Sequence
  Synthetic
cacagtagatgagggagcag

SEQ ID NO: 2288 - DNA, 20 bp, Artificial Sequence
  Synthetic
cacagtagatgagggagcag

SEQ ID NO: 2289 - DNA, 20 bp, Artificial Sequence
  Synthetic
cacagtagatgagggagcag

SEQ ID NO: 2290 - DNA, 20 bp, Artificial Sequence
  Synthetic
acacagtagatgagggagca

SEQ ID NO: 2291 - DNA, 20 bp, Artificial Sequence
  Synthetic
acacagtagatgagggagca

SEQ ID NO: 2292 - DNA, 20 bp, Artificial Sequence
  Synthetic
acacagtagatgagggagca

SEQ ID NO: 2293 - DNA, 20 bp, Artificial Sequence
  Synthetic
cacacagtagatgagggagc

SEQ ID NO: 2294 - DNA, 20 bp, Artificial Sequence
  Synthetic
cacacagtagatgagggagc

SEQ ID NO: 2295 - DNA, 20 bp, Artificial Sequence
  Synthetic
cacacagtagatgagggagc

SEQ ID NO: 2296 - DNA, 20 bp, Artificial Sequence
  Synthetic
gcacacagtagatgagggag

SEQ ID NO: 2297 - DNA, 20 bp, Artificial Sequence
  Synthetic
gcacacagtagatgagggag

SEQ ID NO: 2298 - DNA, 20 bp, Artificial Sequence
  Synthetic
gcacacagtagatgagggag

SEQ ID NO: 2299 - DNA, 20 bp, Artificial Sequence
  Synthetic
tgcacacagtagatgaggga

SEQ ID NO: 2300 - DNA, 20 bp, Artificial Sequence
  Synthetic
tgcacacagtagatgaggga

SEQ ID NO: 2301 - DNA, 20 bp, Artificial Sequence
  Synthetic
tgcacacagtagatgaggga

SEQ ID NO: 2302 - DNA, 20 bp, Artificial Sequence
  Synthetic
gtgcacacagtagatgaggg

SEQ ID NO: 2303 - DNA, 20 bp, Artificial Sequence
  Synthetic
gtgcacacagtagatgaggg

SEQ ID NO: 2304 - DNA, 20 bp, Artificial Sequence
  Synthetic
gtgcacacagtagatgaggg

SEQ ID NO: 2305 - DNA, 20 bp, Artificial Sequence
  Synthetic
agtgcacacagtagaugagg

SEQ ID NO: 2306 - DNA, 20 bp, Artificial Sequence
  Synthetic
agtgcacacagtagaugagg

SEQ ID NO: 2307 - DNA, 20 bp, Artificial Sequence
  Synthetic
agtgcacacagtagaugagg

SEQ ID NO: 2308 - DNA, 20 bp, Artificial Sequence
  Synthetic
gcacacagtagatgagggag

SEQ ID NO: 2309 - DNA, 20 bp, Artificial Sequence
  Synthetic
gcacacagtagatgagggag

SEQ ID NO: 2310 - DNA, 20 bp, Artificial Sequence
  Synthetic
gcacacagtagatgagggag

SEQ ID NO: 2311 - DNA, 20 bp, Artificial Sequence
  Synthetic
ugcacacagtagatgaggga

SEQ ID NO: 2312 - DNA, 20 bp, Artificial Sequence
  Synthetic
ugcacacagtagatgaggga

SEQ ID NO: 2313 - DNA, 20 bp, Artificial Sequence
  Synthetic
ugcacacagtagatgaggga

SEQ ID NO: 2314 - DNA, 20 bp, Artificial Sequence
  Synthetic
gugcacacagtagatgaggg

SEQ ID NO: 2315 - DNA, 20 bp, Artificial Sequence
  Synthetic
gugcacacagtagatgaggg

SEQ ID NO: 2316 - DNA, 20 bp, Artificial Sequence
  Synthetic
gugcacacagtagatgaggg

SEQ ID NO: 2317 - DNA, 20 bp, Artificial Sequence
  Synthetic
agugcacacagtagatgagg

SEQ ID NO: 2318 - DNA, 20 bp, Artificial Sequence
  Synthetic
agugcacacagtagatgagg

SEQ ID NO: 2319 - DNA, 20 bp, Artificial Sequence
  Synthetic
agugcacacagtagatgagg

SEQ ID NO: 2320 - DNA, 20 bp, Artificial Sequence
  Synthetic
gugcacacagtagatgaggg

SEQ ID NO: 2321 - DNA, 20 bp, Artificial Sequence
  Synthetic
gugcacacagtagatgaggg

SEQ ID NO: 2322 - DNA, 20 bp, Artificial Sequence
  Synthetic
gugcacacagtagatgaggg

SEQ ID NO: 2323 - DNA, 20 bp, Artificial Sequence
  Synthetic
gugcacacagtagatgaggg

SEQ ID NO: 2324 - DNA, 20 bp, Artificial Sequence
  Synthetic
gugcacacagtagatgaggg

SEQ ID NO: 2325 - DNA, 20 bp, Artificial Sequence
  Synthetic
gugcacacagtagatgaggg

SEQ ID NO: 2326 - DNA, 20 bp, Artificial Sequence
  Synthetic
gugcacacagtagatgaggg

SEQ ID NO: 2327 - DNA, 20 bp, Artificial Sequence
  Synthetic
gugcacacagtagatgaggg

SEQ ID NO: 2328 - DNA, 20 bp, Artificial Sequence
  Synthetic
gtgcacacagtagatgaggg

SEQ ID NO: 2329 - DNA, 20 bp, Artificial Sequence
  Synthetic
gtgcacacagtagatgaggg

SEQ ID NO: 2330 - DNA, 20 bp, Artificial Sequence
  Synthetic
catgtcacccagcagaccag

SEQ ID NO: 2331 - DNA, 20 bp, Artificial Sequence
  Synthetic
actcacccactcgccaccgc

SEQ ID NO: 2332 - RNA, 20 nt, Artificial Sequence
  Synthetic
ggcacaagggcacagacuuc

SEQ ID NO: 2333 - RNA, 20 nt, Artificial Sequence
  Synthetic
ugcaggcuggcuguuggccc

SEQ ID NO: 2334 - RNA, 20 nt, Artificial Sequence
  Synthetic
ugcaggcuggguguuggccc

SEQ ID NO: 2335 - DNA, 20 bp, Artificial Sequence
  Synthetic
gggccaacagccagcctgca

SEQ ID NO: 2336 - DNA, 20 bp, Artificial Sequence
  Synthetic
gggccaacacccagcctgca

SEQ ID NO: 2337 - RNA, 20 nt, Artificial Sequence
  Synthetic
uugccagguugcagcugcuc

SEQ ID NO: 2338 - RNA, 20 nt, Artificial Sequence
  Synthetic
uugccagguuacagcugcuc

SEQ ID NO: 2339 - DNA, 20 bp, Artificial Sequence
  Synthetic
gagcagctgcaacctggcaa

SEQ ID NO: 2340 - RNA, 20 nt, Artificial Sequence
  Synthetic
uggaagucugugcccuugug

SEQ ID NO: 2341 - RNA, 20 nt, Artificial Sequence
  Synthetic
uggaagucugcgcccuugug

SEQ ID NO: 2342 - DNA, 20 bp, Artificial Sequence
  Synthetic
ctcagtaacattgacaccac

SEQ ID NO: 2343 - DNA, 20 bp, Artificial Sequence
  Synthetic
ctcgactaaagcaggatttc

SEQ ID NO: 2344 - DNA, 20 bp, Artificial Sequence
  Synthetic
cctgcatcagctttatttgt

SEQ ID NO: 2345 - DNA, 20 bp, Artificial Sequence
  Synthetic
tctctattgcacattccaag

SEQ ID NO: 2346 - DNA, 17 bp, Artificial Sequence
  Synthetic
ggcacaagggcacagac

SEQ ID NO: 2347 - DNA, 18 bp, Artificial Sequence
  Synthetic
ggcacaagggcacagact

SEQ ID NO: 2348 - DNA, 19 bp, Artificial Sequence
  Synthetic
ggcacaagggcacagactt

SEQ ID NO: 2349 - DNA, 20 bp, Artificial Sequence
  Synthetic
gttgatctgtagcagcagct

SEQ ID NO: 2350 - DNA, 17 bp, Artificial Sequence
  Synthetic
ggcacaagggcacagac

SEQ ID NO: 2351 - DNA, 18 bp, Artificial Sequence
  Synthetic
ggcacaagggcacagact

SEQ ID NO: 2352 - DNA, 19 bp, Artificial Sequence
  Synthetic
ggcacaagggcacagactt

SEQ ID NO: 2353 - DNA, 17 bp, Artificial Sequence
  Synthetic
ggcacaagggcacagac

SEQ ID NO: 2354 - DNA, 18 bp, Artificial Sequence
  Synthetic
ggcacaagggcacagact

SEQ ID NO: 2355 - DNA, 19 bp, Artificial Sequence
  Synthetic
ggcacaagggcacagactt

SEQ ID NO: 2356 - DNA, 17 bp, Artificial Sequence
  Synthetic
ggcacaagggcacagac

SEQ ID NO: 2357 - DNA, 18 bp, Artificial Sequence
  Synthetic
ggcacaagggcacagact

SEQ ID NO: 2358 - DNA, 19 bp, Artificial Sequence
  Synthetic
ggcacaagggcacagactt